(19) United States
(12) Patent Application Publication
(10) Pub. No.: US 2004/0127446 A1
(43) Pub. Date: Jul. 1, 2004

(54) Oligonucleotide mediated inhibition of hepatitis B virus and hepatitis C virus replication

(76) Inventors: Lawrence Blatt, San Francisco, CA (US); Dennis Macejak, Arvada, CO (US); James McSwiggen, Boulder, CO (US); David Morrissey, Boulder, CO (US); Pamela Pavco, Lafayette, CO (US); Patrice Lee, Erie, CO (US); Kenneth Draper, Reno, NV (US); Elisabeth Roberts, Federal Heights, CO (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/669,841

(22) Filed: Sep. 23, 2003

Related U.S. Application Data
(63) Continuation of application No. PCT/US02/09187, filed on Mar. 26, 2002.
(63) Continuation-in-part of application No. 09/817,879, filed on Mar. 26, 2001.
Application No. 09/817,879 is a continuation-in-part of application No. 09/740,332, filed on Dec. 18, 2000.
Application No. 09/740,332 is a continuation-in-part of application No. 09/611,931, filed on Jul. 7, 2000, now abandoned.
Application No. 09/611,931 is a continuation-in-part of application No. 09/504,231, filed on Feb. 15, 2000, now abandoned.
Application No. 09/504,231 is a continuation-in-part of application No. 09/274,553, filed on Mar. 23, 1999, now abandoned.
Application No. 09/274,553 is a continuation-in-part of application No. 09/257,608, filed on Feb. 25, 1999, now abandoned.
(63) Continuation-in-part of application No. 09/877,478, filed on Jun. 8, 2001, now abandoned.
Application No. 09/877,478 is a continuation-in-part of application No. 09/696,347, filed on Oct. 24, 2000, now abandoned.
Application No. 09/696,347 is a continuation-in-part of application No. 09/636,385, filed on Aug. 9, 2000, now abandoned.
Application No. 09/636,385 is a continuation-in-part of application No. 09/531,025, filed on Mar. 20, 2000, now abandoned.
Application No. 09/531,025 is a continuation-in-part of application No. 09/436,430, filed on Nov. 8, 1999.
Application No. 09/436,430 is a continuation of application No. 08/193,627, filed on Feb. 7, 1994, now Pat. No. 6,017,756.
Application No. 08/193,627 is a continuation of application No. 07/882,712, filed on May 14, 1992, now abandoned.
(60) Provisional application No. 60/296,876, filed on Jun. 8, 2001.
(60) Provisional application No. 60/335,059, filed on Oct. 24, 2001.
(60) Provisional application No. 60/337,055, filed on Dec. 5, 2001.
(60) Provisional application No. 60/358,580, filed on Feb. 20, 2002.
(60) Provisional application No. 60/363,124, filed on Mar. 11, 2002.
(60) Provisional application No. 60/100,842, filed on Sep. 18, 1998.
(60) Provisional application No. 60/083,217, filed on Apr. 27, 1998.

(51) Int. Cl. A61K 48/00; C07H 21/02

(52) U.S. Cl. 514/44; 536/23.72

(57) ABSTRACT

The present invention relates to nucleic acid molecules, including antisense and enzymatic nucleic acid molecules, such as hammerhead ribozymes, DNAzymes, Inozymes, Zinzymes, Amberzymes, and G-cleaver ribozymes, which modulate the synthesis, expression and/or stability of an HCV or HBV RNA and methods for their use alone or in combination with other therapies. In addition, nucleic acid decoy molecules and aptamers that bind to HBV reverse transcriptase and/or HBV reverse transcriptase primer sequences and methods for their use alone or in combination with other therapies, are disclosed. Oligonucleotides that specifically bind the Enhancer I region of HBV DNA are further disclosed. The present invention further relates to the use of nucleic acids, such as decoy and aptamer molecules of the invention, to modulate the expression of Hepatitis B virus (HBV) genes and HBV viral replication. Furthermore, HBV animal models and methods of use are disclosed, including methods of screening for compounds and/or potential therapies directed against HBV. The present invention also relates to compounds, including enzymatic nucleic acid molecules, ribozymes, DNAzymes, nuclease activating compounds and chimeras such as 2′,5′-adenylates, that modulate the expression and/or replication of hepatitis C virus (HCV).

This patent application is a continuation of International Application No. PCT/US02/09187, with international filing date of Mar. 26, 2002, published in English under PCT Article 21(2), which claims the benefit of Macejak et al., USSN (60/296,876), filed Jun. 8, 2001, Macejak et al., USSN (60/335,059), filed Oct. 24, 2001, Morrissey et al., USSN (60/337,055), filed Dec. 5, 2001, Beigelman et al., USSN (60/358,580), filed Feb. 20, 2002, Beigelman et al., USSN (60/363,124), filed Mar. 11, 2002, and which is a continuation-in-part of Blatt et al., USSN (Ser. No. 09/817,879), filed Mar. 26, 2001, which is a continuation-in-part of Blatt et al., USSN (Ser. No. 09/740,332), filed Dec. 18, 2000, which is a continuation-in-part of Blatt et al., USSN (Ser. No. 09/611,931), filed Jul. 7, 2000, which is a continuation-in-part of Blatt et al., USSN (Ser. No. 09/504,321), filed Feb. 15, 2000, which is a continuation-in-part of Blatt et al., USSN (Ser. No. 09/274,553), filed Mar. 23, 1999, which is a continuation-in-part of Blatt et al., USSN (Ser. No. 09/257,608), filed Feb. 24, 1999 (abandoned), which claims the benefit of Blatt et al., USSN (60/100,842), filed Sep. 18, 1998, and McSwiggen et al., USSN (60/083,217) filed Apr. 27, 1998. This patent application is also a continuation-in-part of Draper et al., USSN (Ser. No. 09/877,478) filed Jun. 8, 2001, which is a continuation-in-part of Draper et al., USSN (Ser. No. 09/696,347), filed Oct. 24, 2000, which is a continuation-in-part of Draper et al., USSN (Ser. No. 09/636,385), filed Aug. 9, 2000, which is a continuation-in-part of Draper et al., USSN (Ser. No. 09/531,025), filed Mar. 20, 2000, which is a continuation-in-part of Draper et al., USSN (Ser. No. 09/436,430), filed Nov. 8, 1999, which is a continuation of Draper et al., USSN (Ser. No. 08/193,627), filed Feb. 7, 1994, now U.S. Pat. No. 6,017,756, which is a continuation of Draper et al., USSN (Ser. No. 07/882,712), filed May 14, 1992, now abandoned. All of these listed applications are hereby incorporated by reference herein in their entireties, including the drawings.

### BACKGROUND OF THE INVENTION

The present invention concerns compounds, compositions, and methods for the study, diagnosis, and treatment of degenerative and disease states related to hepatitis B virus (HBV) and hepatitis C virus (HCV) infection, replication and gene expression. Specifically, the invention relates to nucleic acid molecules used to modulate expression of HBV and HCV. In addition, the instant invention relates to methods, models and systems for screening inhibitors of HBV and HCV replication and propagation.

The following is a discussion of relevant art pertaining to hepatitis B virus (HBV) and hepatitis C virus (HCV). The discussion is not meant to be complete and is provided only for understanding of the invention that follows. The summary is not an admission that any of the work described below is prior art to the claimed invention.

In 1989, the Hepatitis C Virus (HCV) was determined to be an RNA virus and was identified as the causative agent of most non-A non-B viral Hepatitis (Choo et al., Science. 1989; 244:359-362). Unlike retroviruses such as HIV, HCV does not go though a DNA replication phase and no integrated forms of the viral genome into the host chromosome have been detected (Houghton et al., Hepatology 1991;14:381-388). Rather, replication of the coding (plus) strand is mediated by the production of a replicative (minus) strand leading to the generation of several copies of plus strand HCV RNA. The genome consists of a single, large, open-reading frame that is translated into a polyprotein (Kato et al., FEBS Letters. 1991; 280: 325-328). This polyprotein subsequently undergoes post-translational cleavage, producing several viral proteins (Leinbach et al., Virology. 1994: 204:163-169).

Examination of the 9.5-kilobase genome of HCV has demonstrated that the viral nucleic acid can mutate at a high rate (Smith et al., Mol. Evol. 1997 45:238-246). This rate of mutation has led to the evolution of several distinct genotypes of HCV that share approximately 70% sequence identity (Simmonds et al., J. Gen. Virol. 1994;75 :1053-1061). It is important to note that these sequences are evolutionarily quite distant. For example, the genetic identity between humans and primates such as the chimpanzee is approximately 98%. In addition, it has been demonstrated that an HCV infection in an individual patient is composed of several distinct and evolving quasispecies that have 98% identity at the RNA level. Thus, the HCV genome is hypervariable and continuously changing. Although the HCV genome is hypervariable, there are 3 regions of the genome that are highly conserved. These conserved sequences occur in the 5′ and 3′ non-coding regions as well as the 5′-end of the core protein coding region and are thought to be vital for HCV RNA replication as well as translation of the HCV polyprotein. Thus, therapeutic agents that target these conserved HCV genomic regions can have a significant impact over a wide range of HCV genotypes. Moreover, it is unlikely that drug resistance will occur with enzymatic nucleic acids specific to conserved regions of the HCV genome. In contrast, therapeutic modalities that target inhibition of enzymes such as the viral proteases or helicase are likely to result in the selection for drug resistant strains since the RNA for these viral encoded enzymes is located in the hypervariable portion of the HCV genome.

After initial exposure to HCV, the patient experiences a transient rise in liver enzymes, which indicates the occurrence of inflammatory processes (Alter et al., IN: Seeff L B, Lewis J H, eds. Current Perspectives in Hepatology. New York: Plenum Medical Book Co; 1989:83-89). This elevation in liver enzymes will occur at least 4 weeks after the initial exposure and can last for up to two months (Farci et al., New England Journal of Medicine. 1991:325:98-104). Prior to the rise in liver enzymes, it is possible to detect HCV RNA in the patient's serum using RT-PCR analysis (Takahashi et al., American Journal of Gastroenterology. 1993:88:2:240-243). This stage of the disease is called the acute stage and usually goes undetected since 75% of patients with acute viral hepatitis from HCV infection are asymptomatic. The remaining 25% of these patients develop jaundice or other symptoms of hepatitis.

Acute HCV infection is a benign disease, however, and as many as 80% of acute HCV patients progress to chronic liver disease as evidenced by persistent elevation of serum alanine aminotransferase (ALT) levels and by continual presence of circulating HCV RNA (Sherlock, Lancet 1992; 339:802). The natural progression of chronic HCV infection over a 10 to 20 year period leads to cirrhosis in 20 to 50% of patients (Davis et al., Infectious Agents and Disease 1993;2:150:154) and progression of HCV infection to hepatocellular carcinoma has been well documented (Liang et al., Hepatology. 1993; 18:1326-1333; Tong et al., Western Journal of Medicine, 1994; Vol. 160, No. 2: 133-138). There have been no studies that have determined sub-populations that are most likely to progress to cirrhosis and/or hepatocellular carcinoma, thus all patients have equal risk of progression.

It is important to note that the survival for patients diagnosed with hepatocellular carcinoma is only 0.9 to 12.8 months from initial diagnosis (Takahashi et al., American Journal of Gastroenterology. 1993:88:2:240-243). Treatment of hepatocellular carcinoma with chemotherapeutic agents has not proven effective and only 10% of patients will benefit from surgery due to extensive tumor invasion of the liver (Trinchet et al., Presse Medicine. 1994:23:831-833). Given the aggressive nature of primary hepatocellular carcinoma, the only viable treatment alternative to surgery is liver transplantation (Pichlmayr et al., Hepatology. 1994:20:33S-40S).

Upon progression to cirrhosis, patients with chronic HCV infection present with clinical features, which are common to clinical cirrhosis regardless of the initial cause (D'Amico et al., Digestive Diseases and Sciences. 1986;31:5: 468-475). These clinical features can include: bleeding esophageal varices, ascites, jaundice, and encephalopathy (Zakim D, Boyer T D. Hepatology a textbook of liver disease. Second Edition Volume 1. 1990 W. B. Saunders Company. Philadelphia). In the early stages of cirrhosis, patients are classified as compensated, meaning that although liver tissue damage has occurred, the patient's liver is still able to detoxify metabolites in the blood-stream. In addition, most patients with compensated liver disease are asymptomatic and the minority with symptoms report only minor symptoms such as dyspepsia and weakness. In the later stages of cirrhosis, patients are classified as decompensated meaning that their ability to detoxify metabolites in the bloodstream is diminished and it is at this stage that the clinical features described above will present.

In 1986, D'Amico et al. described the clinical manifestations and survival rates in 1155 patients with both alcoholic and viral associated cirrhosis (D'Amico supra). Of the 1155 patients, 435 (37%) had compensated disease although 70% were asymptomatic at the beginning of the study. The remaining 720 patients (63%) had decompensated liver disease with 78% presenting with a history of ascites, 31% with jaundice, 17% had bleeding and 16% had encephalopathy. Hepatocellular carcinoma was observed in six (0.5%) patients with compensated disease and in 30 (2.6%) patients with decompensated disease.

Over the course of six years, the patients with compensated cirrhosis developed clinical features of decompensated disease at a rate of 10% per year. In most cases, ascites was the first presentation of decompensation. In addition, hepatocellular carcinoma developed in 59 patients who initially presented with compensated disease by the end of the six-year study.

With respect to survival, the D'Amico study indicated that the five-year survival rate for all patients on the study was only 40%. The six-year survival rate for the patients who initially had compensated cirrhosis was 54%, while the six-year survival rate for patients who initially presented with decompensated disease was only 21%. There were no significant differences in the survival rates between the patients who had alcoholic cirrhosis and the patients with viral related cirrhosis. The major causes of death for the patients in the D'Amico study were liver failure in 49%; hepatocellular carcinoma in 22%; and, bleeding in 13% (D'Amico supra).

Chronic Hepatitis C is a slowly progressing inflammatory disease of the liver, mediated by a virus (HCV) that can lead to cirrhosis, liver failure and/or hepatocellular carcinoma over a period of 10 to 20 years. In the US, it is estimated that infection with HCV accounts for 50,000 new cases of acute hepatitis in the United States each year (NIH Consensus Development Conference Statement on Management of Hepatitis C March 1997). The prevalence of HCV in the United States is estimated at 1.8% and the CDC places the number of chronically infected Americans at approximately 4.5 million people. The CDC also estimates that up to 10,000 deaths per year are caused by chronic HCV infection. The prevalence of HCV in the United States is estimated at 1.8% and the CDC places the number of chronically infected Americans at approximately 4.5 million people. The CDC also estimates that up to 10,000 deaths per year are caused by chronic HCV infection.

Numerous well controlled clinical trials using interferon (IFN-alpha) in the treatment of chronic HCV infection have demonstrated that treatment three times a week results in lowering of serum ALT values in approximately 50% (range 40% to 70%) of patients by the end of 6 months of therapy (Davis et al., New England Journal of Medicine 1989; 321:1501-1506; Marcellin et al., Hepatology. 1991; 13:393-397; Tong et al., Hepatology 1997:26:747-754; Tong et al., Hepatology 1997 26(6): 1640-1645). However, following cessation of interferon treatment, approximately 50% of the responding patients relapsed, resulting in a “durable” response rate as assessed by normalization of serum ALT concentrations of approximately 20 to 25%.

In recent years, direct measurement of the HCV RNA has become possible through use of either the branched-DNA or Reverse Transcriptase Polymerase Chain Reaction (RT-PCR) analysis. In general, the RT-PCR methodology is more sensitive and leads to more accurate assessment of the clinical course (Tong et al., supra). Studies that have examined six months of type 1 interferon therapy using changes in HCV RNA values as a clinical endpoint have demonstrated that up to 35% of patients will have a loss of HCV RNA by the end of therapy (Marcellin et al., supra). However, as with the ALT endpoint, about 50% of the patients relapse six months following cessation of therapy resulting in a durable virologic response of only 12% (Marcellin et al., supra). Studies that have examined 48 weeks of therapy have demonstrated that the sustained virological response is up to 25% (NIH consensus statement: 1997). Thus, standard of care for treatment of chronic HCV infection with type 1 interferon is now 48 weeks of therapy using changes in HCV RNA concentrations as the primary assessment of efficacy (Hoofnagle et al., New England Journal of Medicine 1997; 336(5) 347-356).

Side effects resulting from treatment with type 1 interferons can be divided into four general categories, which include 1. Influenza-like symptoms; 2. Neuropsychiatric; 3. Laboratory abnormalities; and, 4. Miscellaneous (Dusheiko et al., Journal of Viral Hepatitis. 1994:1:3-5). Examples of influenza-like symptoms include; fatigue, fever; myalgia; malaise; appetite loss; tachycardia; rigors; headache and arthralgias. The influenza-like symptoms are usually short-lived and tend to abate after the first four weeks of dosing (Dushieko et al., supra). Neuropsychiatric side effects include: irritability, apathy; mood changes; insomnia; cognitive changes and depression. The most important of these neuropsychiatric side effects is depression and patients who have a history of depression should not be given type 1 interferon. Laboratory abnormalities include; reduction in myeloid cells including granulocytes, platelets and to a lesser extent red blood cells. These changes in blood cell counts rarely lead to any significant clinical sequellae (Dushieko et al., supra). In addition, increases in triglyceride concentrations and elevations in serum alanine and aspartate aminotransferase concentration have been observed. Finally, thyroid abnormalities have been reported. These thyroid abnormalities are usually reversible after cessation of interferon therapy and can be controlled with appropriate medication while on therapy. Miscellaneous side effects include nausea; diarrhea; abdominal and back pain; pruritus; alopecia; and rhinorrhea. In general, most side effects will abate after 4 to 8 weeks of therapy (Dushieko et al., supra).

Type 1 Interferon is a key constituent of many treatment programs for chronic HCV infection. Treatment with type 1 interferon induces a number of genes and results in an antiviral state within the cell. One of the genes induced is 2′,5′ oligoadenylate synthetase, an enzyme that synthesizes short 2′,5′ oligoadenylate (2-5A) molecules. Nascent 2-5A subsequently activates a latent RNase, RNase L, which in turn nonspecifically degrades viral RNA.

Chronic hepatitis B is caused by an enveloped virus, commonly known as the hepatitis B virus or HBV. HBV is transmitted via infected blood or other body fluids, especially saliva and semen, during delivery, sexual activity, or sharing of needles contaminated by infected blood. Individuals may be “carriers” and transmit the infection to others without ever having experienced symptoms of the disease. Persons at highest risk are those with multiple sex partners, those with a history of sexually transmitted diseases, parenteral drug users, infants born to infected mothers, “close” contacts or sexual partners of infected persons, and healthcare personnel or other service employees who have contact with blood. Transmission is also possible via tattooing, ear or body piercing, and acupuncture; the virus is also stable on razors, toothbrushes, baby bottles, eating utensils, and some hospital equipment such as respirators, scopes and instruments. There is no evidence that HBsAg positive food handlers pose a health risk in an occupational setting, nor should they be excluded from work. Hepatitis B has never been documented as being a food-borne disease. The average incubation period is 60 to 90 days, with a range of 45 to 180; the number of days appears to be related to the amount of virus to which the person was exposed. However, determining the length of incubation is difficult, since onset of symptoms is insidious. Approximately 50% of patients develop symptoms of acute hepatitis that last from 1 to 4 weeks. Two percent or less of these individuals develop fulminant hepatitis resulting in liver failure and death.

The determinants of severity include: (1) The size of the dose to which the person was exposed; (2) the person's age with younger patients experiencing a milder form of the disease; (3) the status of the immune system with those who are immunosuppressed experiencing milder cases; and (4) the presence or absence of co-infection with the Delta virus (hepatitis D), with more severe cases resulting from co-infection. In symptomatic cases, clinical signs include loss of appetite, nausea, vomiting, abdominal pain in the right upper quadrant, arthralgia, and tiredness/loss of energy. Jaundice is not experienced in all cases, however, jaundice is more likely to occur if the infection is due to transfusion or percutaneous serum transfer, and it is accompanied by mild pruritus in some patients. Bilirubin elevations are demonstrated in dark urine and clay-colored stools, and liver enlargement may occur accompanied by right upper-quadrant pain. The acute phase of the disease may be accompanied by severe depression, meningitis, Guillain-Barré syndrome, myelitis, encephalitis, agranulocytosis, and/or thrombocytopenia.

Hepatitis B is generally self-limiting and will resolve in approximately 6 months. Asymptomatic cases can be detected by serologic testing, since the presence of the virus leads to production of large amounts of HBsAg in the blood. This antigen is the first and most useful diagnostic marker for active infections. However, if HBsAg remains positive for 20 weeks or longer, the person is likely to remain positive indefinitely and is now a carrier. While only 10% of persons over age 6 who contract HBV become carriers, 90% of infants infected during the first year of life do so.

Hepatitis B virus (HBV) infects over 300 million people worldwide (Imperial, 1999, Gastroenterol. Hepatol., 14 (suppl), Si-5). In the United States, approximately 1.25 million individuals are chronic carriers of HBV as evidenced by the fact that they have measurable hepatitis B virus surface antigen HBsAg in their blood. The risk of becoming a chronic HBsAg carrier is dependent upon the mode of acquisition of infection as well as the age of the individual at the time of infection. For those individuals with high levels of viral replication, chronic active hepatitis with progression to cirrhosis, liver failure and hepatocellular carcinoma (HCC) is common, and liver transplantation is the only treatment option for patients with end-stage liver disease from HBV.

The natural progression of chronic HBV infection over a 10 to 20 year period leads to cirrhosis in 20-to-50% of patients and progression of HBV infection to hepatocellular carcinoma has been well documented. There have been no studies that have determined sub-populations that are most likely to progress to cirrhosis and/or hepatocellular carcinoma, thus all patients have equal risk of progression.

It is important to note that the survival for patients diagnosed with hepatocellular carcinoma is only 0.9 to 12.8 months from initial diagnosis (Takahashi et al., 1993, American Journal of Gastroenterology, 88, 240-243). Treatment of hepatocellular carcinoma with chemotherapeutic agents has not proven effective and only 10% of patients will benefit from surgery due to extensive tumor invasion of the liver (Trinchet et al., 1994, Presse Medicine, 23, 831-833). Given the aggressive nature of primary hepatocellular carcinoma, the only viable treatment alternative to surgery is liver transplantation (Pichlmayr et al., 1994, Hepatology., 20, 33S-40S).

Upon progression to cirrhosis, patients with chronic HCV and HBV infection present with clinical features, which are common to clinical cirrhosis regardless of the initial cause (D'Amico et al., 1986, Digestive Diseases and Sciences, 31, 468-475). These clinical features may include: bleeding esophageal varices, ascites, jaundice, and encephalopathy (Zakim D, Boyer T D. Hepatology a textbook of liver disease, Second Edition Volume 1. 1990 W.B. Saunders Company. Philadelphia). In the early stages of cirrhosis, patients are classified as compensated, meaning that although liver tissue damage has occurred, the patient's liver is still able to detoxify metabolites in the blood-stream. In addition, most patients with compensated liver disease are asymptomatic and the minority with symptoms report only minor symptoms such as dyspepsia and weakness. In the later stages of cirrhosis, patients are classified as decompensated meaning that their ability to detoxify metabolites in the bloodstream is diminished and it is at this stage that the clinical features described above will present.

In 1986, D'Amico et al. described the clinical manifestations and survival rates in 1155 patients with both alcoholic and viral associated cirrhosis (D'Amico supra). Of the 1155 patients, 435 (37%) had compensated disease although 70% were asymptomatic at the beginning of the study. The remaining 720 patients (63%) had decompensated liver disease with 78% presenting with a history of ascites, 31% with jaundice, 17% had bleeding and 16% had encephalopathy. Hepatocellular carcinoma was observed in six (0.5%) patients with compensated disease and in 30 (2.6%) patients with decompensated disease.

Over the course of six years, the patients with compensated cirrhosis developed clinical features of decompensated disease at a rate of 10% per year. In most cases, ascites was the first presentation of decompensation. In addition, hepatocellular carcinoma developed in 59 patients who initially presented with compensated disease by the end of the six-year study.

With respect to survival, the D'Amico study indicated that the five-year survival rate for all patients on the study was only 40%. The six-year survival rate for the patients who initially had compensated cirrhosis was 54% while the six-year survival rate for patients who initially presented with decompensated disease was only 21%. There were no significant differences in the survival rates between the patients who had alcoholic cirrhosis and the patients with viral related cirrhosis. The major causes of death for the patients in the D'Amico study were liver failure in 49%; hepatocellular carcinoma in 22%; and, bleeding in 13% (D'Amico supra).

Hepatitis B virus is a double-stranded circular DNA virus. It is a member of the Hepadnaviridae family. The virus consists of a central core that contains a core antigen (HBcAg) surrounded by an envelope containing a surface protein/surface antigen (HBsAg) and is 42 nm in diameter. It also contains an e antigen (HBeAg), which, along with HBcAg and HBsAg, is helpful in identifying this disease.

In HBV virions, the genome is found in an incomplete double-stranded form. HBV uses a reverse transcriptase to transcribe a positive-sense full length RNA version of its genome back into DNA. This reverse transcriptase also contains DNA polymerase activity and thus begins replicating the newly synthesized minus-sense DNA strand. However, it appears that the core protein encapsidates the reverse-transcriptase/polymerase before it completes replication.

From the free-floating form, the virus must first attach itself specifically to a host cell membrane. Viral attachment is one of the crucial steps that determines host and tissue specificity. However, currently there are no in vitro cell-lines that can be infected by HBV. There are some cells lines, such as HepG2, which can support viral replication only upon transient or stable transfection using HBV DNA.

After attachment, fusion of the viral envelope and host membrane must occur to allow the viral core proteins containing the genome and polymerase to enter the cell. Once inside, the genome is translocated to the nucleus where it is repaired and cyclized.

The complete closed circular DNA genome of HBV remains in the nucleus and gives rise to four transcripts. These transcripts initiate at unique sites but share the same 3′-ends. The 3.5-kb pregenomic RNA serves as a template for reverse transcription and also encodes the nucleocapsid protein and polymerase. A subclass of this transcript with a 5′-end extension codes for the precore protein that, after processing, is secreted as HBV e antigen. The 2.4-kb RNA encompasses the pre-S1 open reading frame (ORF) that encodes the large surface protein. The 2.1-kb RNA encompasses the pre-S2 and S ORFs that encode the middle and small surface proteins, respectively. The smallest transcript (˜0.8-kb) codes for the X protein, a transcriptional activator.

Multiplication of the HBV genome begins within the nucleus of an infected cell. RNA polymerase II transcribes the circular HBV DNA into greater-than-full length mRNA. Since the mRNA is longer than the actual complete circular DNA, redundant ends are formed. Once produced, the pregenomic RNA exits the nucleus and enters the cytoplasm.

The packaging of pregenomic RNA into core particles is triggered by the binding of the HBV polymerase to the 5′ epsilon stem-loop. RNA encapsidation is believed to occur as soon as binding occurs. The HBV polymerase also appears to require associated core protein in order to function. The HBV polymerase initiates reverse transcription from the 5′ epsilon stem-loop three to four base pairs at which point the polymerase and attached nascent DNA are transferred to the 3′ copy of the DR1 region. Once there, the (−)DNA is extended by the HBV polymerase while the RNA template is degraded by the HBV polymerase RNAse H activity. When the HBV polymerase reaches the 5′ end, a small stretch of RNA is left undigested by the RNAse H activity. This segment of RNA is comprised of a small sequence just upstream and including the DR1 region. The RNA oligomer is then translocated and annealed to the DR2 region at the 5′ end of the (−)DNA. It is used as a primer for the (+)DNA synthesis which is also generated by the HBV polymerase. It appears that the reverse transcription as well as plus strand synthesis may occur in the completed core particle.

Since the pregenomic RNA is required as a template for DNA synthesis, this RNA is an excellent target for nucleic acid based therapeutics. Nucleoside analogues that have been documented to modulate HBV replication target the reverse transcriptase activity needed to convert the pregenomic RNA into DNA. Nucleic acid decoy and aptamer modulation of HBV reverse transcriptase would be expected to result in a similar modulation of HBV replication.

Current therapeutic goals of treatment are three-fold: to eliminate infectivity and transmission of HBV to others, to arrest the progression of liver disease and improve the clinical prognosis, and to prevent the development of hepatocellular carcinoma (HCC).

Interferon alpha use is the most common therapy for HBV; however-, recently Lamivudine (3TC®) has been approved by the FDA. Interferon alpha (IFN-alpha) is one treatment for chronic hepatitis B. The standard duration of IFN-alpha therapy is 16 weeks, however, the optimal treatment length is still poorly defined. A complete response (HBV DNA negative HBeAg negative) occurs in approximately 25% of patients. Several factors have been identified that predict a favorable response to therapy including: High ALT, low HBV DNA, being female, and heterosexual orientation.

There is also a risk of reactivation of the hepatitis B virus even after a successful response, this occurs in around 5% of responders and normally occurs within 1 year.

Side effects resulting from treatment with type 1 interferons can be divided into four general categories including: Influenza-like symptoms, neuropsychiatric, laboratory abnormalities, and other miscellaneous side effects. Examples of influenza-like symptoms include, fatigue, fever, myalgia, malaise, appetite loss, tachycardia, rigors, headache and arthralgias. The influenza-like symptoms are usually short-lived and tend to abate after the first four weeks of dosing (Dusheiko et al., 1994, Journal of Viral Hepatitis, 1, 3-5). Neuropsychiatric side effects include irritability, apathy, mood changes, insomnia, cognitive changes, and depression. Laboratory abnormalities include the reduction of myeloid cells, including granulocytes, platelets and to a lesser extent, red blood cells. These changes in blood cell counts rarely lead to any significant clinical sequellae. In addition, increases in triglyceride concentrations and elevations in serum alanine and aspartate aminotransferase concentration have been observed. Finally, thyroid abnormalities have been reported. These thyroid abnormalities are usually reversible after cessation of interferon therapy and can be controlled with appropriate medication while on therapy. Miscellaneous side effects include nausea, diarrhea, abdominal and back pain, pruritus, alopecia, and rhinorrhea. In general, most side effects will abate after 4 to 8 weeks of therapy (Dushieko et al, supra).

Lamivudine (3TC®) is a nucleoside analogue, which is a very potent and specific inhibitor of HBV DNA synthesis. Lamivudine has recently been approved for the treatment of chronic Hepatitis B. Unlike treatment with interferon, treatment with 3TC® does not eliminate the HBV from the patient. Rather, viral replication is controlled and chronic administration results in improvements in liver histology in over 50% of patients. Phase III studies with 3TC®, showed that treatment for one year was associated with reduced liver inflammation and a delay in scarring of the liver. In addition, patients treated with Lamivudine (100 mg per day) had a 98 percent reduction in hepatitis B DNA and a significantly higher rate of seroconversion, suggesting disease improvements after completion of therapy. However, stopping of therapy resulted in a reactivation of HBV replication in most patients. In addition recent reports have documented 3TC® resistance in approximately 30% of patients.

Current therapies for treating HBV infection, including interferon and nucleoside analogues, are only partially effective. In addition, drug resistance to nucleoside analogues is now emerging, making treatment of chronic Hepatitis B more difficult. Thus, a need exists for effective treatment of this disease that utilizes antiviral modulators that work by mechanisms other than those currently utilized in the treatment of both acute and chronic hepatitis B infections.

Welch et al., Gene Therapy 1996 3(11): 994-1001 describe in vitro an in vivo studies with two vector expressed hairpin ribozymes targeted against hepatitis C virus.

Sakamoto et al., J. Clinical Investigation 1996 98(12): 2720-2728 describe intracellular cleavage of hepatitis C virus RNA and inhibition of viral protein translation by certain vector expressed hammerhead ribozymes.

Lieber et al., J. Virology 1996 70(12): 8782-8791 describe elimination of hepatitis C virus RNA in infected human hepatocytes by adenovirus-mediated expression of certain hammerhead ribozymes.

Ohkawa et al., 1997, J. Hepatology, 27; 78-84, describe in vitro cleavage of HCV RNA and inhibition of viral protein translation using certain in vitro transcribed hammerhead ribozymes.

Barber et al., International PCT Publication No. WO 97/32018, describe the use of an adenovirus vector to express certain anti-hepatitis C virus hairpin ribozymes.

Kay et al., International PCT Publication No. WO 96/18419, describe certain recombinant adenovirus vectors to express anti-HCV hammerhead ribozymes.

Yamada et al., Japanese Patent Application No. JP 07231784 describe a specific poly-(L)-lysine conjugated hammerhead ribozyme targeted against HCV.

Draper, U.S. Pat. Nos. 5,610,054 and 5,869,253, describes enzymatic nucleic acid molecules capable of inhibiting replication of HCV.

Macejak et al., 2000, Hepatology, 31, 769-776, describe enzymatic nucleic acid molecules capable of inhibiting replication of HCV.

Weifeng and Torrence, 1997, Nucleosides and Nucleotides, 16, 7-9, describe the synthesis of 2-5A antisense chimeras with various non-nucleoside components.

Torrence et al., U.S. Pat. No. 5,583,032 describe targeted cleavage of RNA using an antisense oligonulceotide linked to a 2′,5′-oligoadenylate activator of RNase L.

Suhadolnik and Pfleiderer, U.S. Pat. Nos. 5,863,905; 5,700,785; 5,643,889; 5,556,840; 5,550,111; 5,405,939; 5,188,897; 4,924,624; and 4,859,768 describe specific internucleotide phosphorothioate 2′,5′-oligoadenlyates and 2′,5′-oligoadenlyate conjugates.

Budowsky et al., U.S. Pat. No. 5,962,431 describe a method of treating papillomavirus using specific 2′,5′-oligoadenylates.

Torrence et al., International PCT publication No. WO 00/14219, describe specific peptide nucleic acid 2′,5′-oligoadenylate chimeric molecules.

Stinchcomb et al., U.S. Pat. No. 5,817,796, describe C-myb ribozymes having 2′-5′-Linked Adenylate Residues.

Draper, U.S. Pat. No. 6,017,756, describes the use of ribozymes for the inhibition of Hepatitis B Virus.

Passman et al., 2000, Biochem. Biophys. Res. Commun., 268(3), 728-733.; Gan et al., 1998, J. Med. Coll. PLA, 13(3), 157-159.; Li et al., 1999, Jiefangiun Yixue Zazhi, 24(2), 99-101.; Putlitz et al., 1999, J. Virol., 73(7), 5381-5387.; Kim et al., 1999, Biochem. Biophys. Res. Commun., 257(3), 759-765.; Xu et al., 1998, Bingdu Xuebao, 14(4), 365-369.; Welch et al., 1997, Gene Ther., 4(7), 736-743.; Goldenberg et al., 1997, International PCT publication No. WO 97/08309, Wands et al., 1997, J. of Gastroenterology and Hepatology, 12(suppl.), S354-S369.; Ruiz et al., 1997, BioTechniques, 22(2), 338-345.; Gan et al., 1996, J. Med. Coll. PLA, 11(3), 171-175.; Beck and Nassal, 1995, Nucleic Acids Res., 23(24), 4954-62.; Goldenberg, 1995, International PCT publication No. WO 95/22600.; Xu et al., 1993, Bingdu Xuebao, 9(4), 331-6.; Wang et al., 1993, Bingdu Xuebao, 9(3), 278-80, all describe ribozymes that are targeted to cleave a specific HBV target site.

Hunt et al., U.S. Pat. No. 5,859,226, describes specific non-naturally occurring oligonucleotide decoys intended to inhibit the expression of MHC-II genes through binding of the RF-X transcription factor, that can inhibit the expression of certain HBV and CMV viral proteins.

Kao et al., International PCT Publication No. WO 00/04141, describes linear single stranded nucleic acid molecules capable of specifically binding to viral polymerases and inhibiting the activity of the viral polymerase.

Lu, International PCT Publication No. WO 99/20641, describes specific triplex-forming oligonucleotides used in treating HBV infection.

### SUMMARY OF THE INVENTION

This invention relates to enzymatic nucleic acid molecules that can disrupt the function of RNA species of hepatitis B virus (HBV), hepatitis C virus (HCV) and/or those RNA species encoded by HBV or HCV. In particular, applicant provides enzymatic nucleic acid molecules capable of specifically cleaving HBV RNA or HCV RNA and describes the selection and function thereof. Such enzymatic nucleic acid molecules can be used to treat diseases and disorders associated with HBV and HCV infection.

In one embodiment, the invention features an enzymatic nucleic acid molecule that specifically cleaves RNA derived from hepatitis B virus (HBV), wherein the enzymatic nucleic acid molecule comprises sequence defined as Seq. ID No. 10887.

In another embodiment, the invention features a composition comprising an enzymatic nucleic acid molecule of the invention and a pharmaceutically acceptable carrier.

In another embodiment, the invention features a mammalian cell, for example a human cell, comprising an enzymatic nucleic acid molecule contemplated by the invention.

In one embodiment, the invention features a method for the treatment of cirrhosis, liver failure or hepatocellular carcinoma comprising administering to a patient an enzymatic nucleic acid molecule of the invention under conditions suitable for the treatment.

In another embodiment, the invention features a method for the treatment of a patient having a condition associated with HBV and/or HCV infection, comprising contacting cells of said patient with an enzymatic nucleic acid molecule of the invention.

In another embodiment, the invention features a method for the treatment of a patient having a condition associated with HBV and/or HCV infection, comprising contacting cells of said patient with an enzymatic nucleic acid molecule of the invention and further comprising the use of one or more drug therapies, for example, type I interferon or 3TC® (lamivudine), under conditions suitable for said treatment. In another embodiment, the other therapy is administered simultaneously with or separately from the enzymatic nucleic acid molecule.

In another embodiment, the invention features a method for inhibiting HBV and/or HCV replication in a mammalian cell comprising administering to the cell an enzymatic nucleic acid molecule of the invention under conditions suitable for the inhibition.

In yet another embodiment, the invention features a method of cleaving a separate HBV and/or HCV RNA comprising contacting an enzymatic nucleic acid molecule of the invention with the separate RNA under conditions suitable for the cleavage of the separate RNA.

In one embodiment, cleavage by an enzymatic nucleic acid molecule of the invention is carried out in the presence of a divalent cation, for example Mg2+.

In another embodiment, the enzymatic nucleic acid molecule of the invention is chemically synthesized.

In another embodiment, the type I interferon contemplated by the invention is interferon alpha, interferon beta, polyethylene glycol interferon, polyethylene glycol interferon alpha 2a, polyethylene glycol interferon alpha 2b, polyethylene glycol consensus interferon.

In one embodiment, the invention features a composition comprising type I interferon and an enzymatic nucleic acid molecule of the invention and a pharmaceutically acceptable carrier.

In another embodiment, the invention features a method of administering to a cell, for example a mammalian cell or human cell, an enzymatic nucleic acid molecule of the invention independently or in conjunction with other therapeutic compounds, such as type I interferon or 3TC® (lamivudine), comprising contacting the cell with the enzymatic nucleic acid molecule under conditions suitable for the administration.

In another embodiment, administration of an enzymatic nucleic acid molecule of the invention is in the presence of a delivery reagent, for example a lipid, cationic lipid, phospholipid, or liposome.

In another embodiment, the invention features novel nucleic acid-based techniques such as enzymatic nucleic acid molecules and antisense molecules and methods for their use to down regulate or inhibit the expression of HBV RNA and/or replication of HBV.

In another embodiment, the invention features novel nucleic acid-based techniques such as enzymatic nucleic acid molecules and antisense molecules and methods for their use to down regulate or inhibit the expression of HCV RNA and/or replication of HCV.

In one embodiment, the invention features the use of one or more of the enzymatic nucleic acid-based techniques to down-regulate or inhibit the expression of the genes encoding HBV and/or HCV viral proteins. Specifically, the invention features the use of enzymatic nucleic acid-based techniques to specifically down-regulate or inhibit the expression of the HBV and/or HCV viral genome.

In another embodiment, the invention features nucleic acid-based inhibitors (e.g., enzymatic nucleic acid molecules (ribozymes), antisense nucleic acids, triplex DNA, decoys, siRNA, aptamers, and antisense nucleic acids containing RNA cleaving chemical groups) and methods for their use to down regulate or inhibit the expression of RNA (e.g., HBV and/or HCV) capable of progression and/or maintenance of hepatitis, hepatocellular carcinoma, cirrhosis, and/or liver failure.

In one embodiment, nucleic acid molecules of the invention are used to treat HBV infected cells or an HBV infected patient wherein the HBV is resistant or the patient does not respond to treatment with 3TC® (Lamivudine), either alone or in combination with other therapies under conditions suitable for the treatment.

In yet another embodiment, the invention features the use of an enzymatic nucleic acid molecule, preferably in the hammerhead, NCH (Inozyme), G-cleaver, amberzyme, zinzyme, and/or DNAzyme motif, to inhibit the expression of HBV and/or HCV RNA.

The enzymatic nucleic acid molecules described herein exhibit a high degree of specificity for only the viral mRNA in infected cells. Nucleic acid molecules of the instant invention targeted to highly conserved sequence regions allow the treatment of many strains of human HBV and/or HCV with a single compound. No treatment presently exists which specifically attacks expression of the viral gene(s) that are responsible for transformation of hepatocytes by HBV and/or HCV.

The enzymatic nucleic acid-based modulators of HBV and HCV expression are useful for the prevention of the diseases and conditions including HBV and HCV infection, hepatitis, cancer, cirrhosis, liver failure, and any other diseases or conditions that are related to the levels of HBV and/or HCV in a cell or tissue.

Preferred target sites are genes required for viral replication, a non-limiting example includes genes for protein synthesis, such as the 5′ most 1500 nucleotides of the HBV pregenomic mRNAs. For sequence references, see Renbao et al., 1987, Sci. Sin., 30, 507. This region controls the translational expression of the core protein (C), X protein (X) and DNA polymerase (P) genes and plays a role in the replication of the viral DNA by serving as a template for reverse transcriptase. Disruption of this region in the RNA results in deficient protein synthesis as well as incomplete DNA synthesis (and inhibition of transcription from the defective genomes). Targeting sequences 5′ of the encapsidation site can result in the inclusion of the disrupted 3′ RNA within the core virion structure and targeting sequences 3′ of the encapsidation site can result in the reduction in protein expression from both the 3′ and 5′ fragments.

Alternative regions outside of the 5′ most 1500 nucleotides of the pregenomic mRNA also make suitable targets for enzymatic nucleic acid mediated inhibition of HBV replication. Such targets include the mRNA regions that encode the viral S gene. Selection of particular target regions will depend upon the secondary structure of the pregenomic mRNA. Targets in the minor mRNAs can also be used, especially when folding or accessibility assays in these other RNAs reveal additional target sequences that are unavailable in the pregenomic mRNA species.

A desirable target in the pregenomic RNA is a proposed bipartite stem-loop structure in the 3′-end of the pregenomic RNA which is believed to be critical for viral replication (Kidd and Kidd-Ljunggren, 1996. Nuc. Acid Res. 24:3295-3302). The 5′end of the HBV pregenomic RNA carries a cis-acting encapsidation signal, which has inverted repeat sequences that are thought to form a bipartite stem-loop structure. Due to a terminal redundancy in the pregenomic RNA, the putative stem-loop also occurs at the 3′-end. While it is the 5′ copy which functions in polymerase binding and encapsidation, reverse transcription actually begins from the 3′ stem-loop. To start reverse transcription, a 4 nt primer which is covalently attached to the polymerase is made, using a bulge in the 5′ encapsidation signal as template. This primer is then shifted, by an unknown mechanism, to the DR1 primer binding site in the 3′ stem-loop structure, and reverse transcription proceeds from that point. The 3′ stem-loop, and especially the DR1 primer binding site, appear to be highly effective targets for ribozyme intervention.

Sequences of the pregenomic RNA are shared by the mRNAs for surface, core, polymerase, and X proteins. Due to the overlapping nature of the HBV transcripts, all share a common 3′-end. Enzymatic nucleic acids targeting of this common 3-end will thus cleave the pregenomic RNA as well as all of the mRNAs for surface, core, polymerase and X proteins.

At least seven basic varieties of naturally-occurring enzymatic RNAs are known presently. Each can catalyze the hydrolysis of RNA phosphodiester bonds in trans (and thus can cleave other RNA molecules) under physiological conditions. Table I summarizes some of the characteristics of these enzymatic RNA molecules. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single enzymatic nucleic acid molecule is able to cleave many molecules of target RNA. In addition, the enzymatic nucleic acid is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a an enzymatic nucleic acid molecule.

The enzymatic nucleic acid molecules that cleave the specified sites in HBV-specific RNAs represent a novel therapeutic approach to treat a variety of pathologic indications, including, HBV infection, hepatitis, hepatocellular carcinoma, tumorigenesis, cirrhosis, liver failure and other conditions related to the level of HBV.

In one of the preferred embodiments of the inventions described herein, the enzymatic nucleic acid molecule is formed in a hammerhead or hairpin motif, but can also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183. Examples of hairpin motifs are described by Hampel et al., EP0360257, Hampel and Tritz, 1989 Biochemistry 28, 4929, Feldstein et al., 1989, Gene 82, 53, Haseloff and Gerlach, 1989, Gene, 82, 43, Hampel et al., 1990 Nucleic Acids Res. 18, 299; and Chowrira & McSwiggen, U.S. Pat. No. 5,631,359. The hepatitis delta virus motif is described by Perrotta and Been, 1992 Biochemistry 31, 16. The RNase P motif is described by Guerrier-Takada et al., 1983 Cell 35, 849; Forster and Altman, 1990, Science 249, 783; and Li and Altman, 1996, Nucleic Acids Res. 24, 835. The Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; and Guo and Collins, 1995, EMBO. J. 14, 363). Group II introns are described by Griffin et al., 1995, Chem. Biol. 2, 761; Michels and Pyle, 1995, Biochemistry 34, 2965; and Pyle et al., International PCT Publication No. WO 96/22689. The Group I intron is described by Cech et al., U.S. Pat. No. 4,987,071. DNAzymes are described by Usman et al., International PCT Publication No. WO 95/11304; Chartrand et al., 1995, NAR 23, 4092; Breaker et al., 1995, Chem. Bio. 2, 655; and Santoro et al., 1997, PNAS 94, 4262. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs include the Aptazyme (Breaker et al., WO 98/43993), Amberzyme (Class I motif; FIG. 3; Beigelman et al., International PCT publication No. WO 99/55857) and Zinzyme (Beigelman et al., International PCT publication No. WO 99/55857), all these references are incorporated by reference herein in their totalities, including drawings and can also be used in the present invention. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

In preferred embodiments of the present invention, a nucleic acid molecule, e.g., an antisense molecule, a triplex DNA, or a ribozyme, is 13 to 100 nucleotides in length, e.g., in specific embodiments 35, 36, 37, or 38 nucleotides in length (e.g., for particular ribozymes or antisense). In particular embodiments, the nucleic acid molecule is 15-100, 17-100, 20-100, 21-100, 23-100, 25-100, 27-100, 30-100, 32-100, 35-100, 40-100, 50-100, 60-100, 70-100, or 80-100 nucleotides in length. Instead of 100 nucleotides being the upper limit on the length ranges specified above, the upper limit of the length range can be, for example, 30, 40, 50, 60, 70, or 80 nucleotides. Thus, for any of the length ranges, the length range for particular embodiments has lower limit as specified, with an upper limit as specified which is greater than the lower limit. For example, in a particular embodiment, the length range can be 35-50 nucleotides in length. All such ranges are expressly included. Also in particular embodiments, a nucleic acid molecule can have a length which is any of the lengths specified above, for example, 21 nucleotides in length.

Exemplary enzymatic nucleic acid molecules of the invention targeting HBV are shown in Tables V-XI. For example, enzymatic nucleic acid molecules of the invention are preferably between 15 and 50 nucleotides in length, more preferably between 25 and 40 nucleotides in length, e.g., 34, 36, or 38 nucleotides in length (for example see Jarvis et al., 1996, J. Biol. Chem., 271, 29107-29112). Exemplary DNAzymes of the invention are preferably between 15 and 40 nucleotides in length, more preferably between 25 and 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Exemplary antisense molecules of the invention are preferably between 15 and 75 nucleotides in length, more preferably between 20 and 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). Exemplary triplex forming oligonucleotide molecules of the invention are preferably between 10 and 40 nucleotides in length, more preferably between 12 and 25 nucleotides in length, e.g., 18, 19, 20, or 21 nucleotides in length (see for example Maher et al., 1990, Biochemistry, 29, 8820-8826; Strobel and Dervan, 1990, Science, 249, 73-75). Those skilled in the art will recognize that all that is required is for the nucleic acid molecule are of length and conformation sufficient and suitable for the nucleic acid molecule to catalyze a reaction contemplated herein. The length of the nucleic acid molecules of the instant invention are not limiting within the general limits stated.

In a preferred embodiment, the invention provides a method for producing a class of nucleic acid-based gene inhibiting agents which exhibit a high degree of specificity for the RNA of a desired target. For example, the enzymatic nucleic acid molecule is preferably targeted to a highly conserved sequence region of target RNAs encoding HBV proteins (specifically HBV RNA) such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., ribozymes and antisense) can be expressed from DNA and/or RNA vectors that are delivered to specific cells.

The enzymatic nucleic acid-based inhibitors of HBV expression are useful for the prevention of the diseases and conditions including HBV infection, hepatitis, cancer, cirrhosis, liver failure, and any other diseases or conditions that are related to the levels of HBV in a cell or tissue.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers. In preferred embodiments, the enzymatic nucleic acid HBV inhibitors comprise sequences, which are complementary to the substrate sequences in Tables IV to XI. Examples of such enzymatic nucleic acid molecules also are shown in Tables V to XI. Examples of such enzymatic nucleic acid molecules consist essentially of sequences defined in these tables.

In yet another embodiment, the invention features antisense nucleic acid molecules including sequences complementary to the HBV substrate sequences shown in Tables IV to XI. Such nucleic acid molecules can include sequences as shown for the binding arms of the enzymatic nucleic acid molecules in Tables V to XI. Similarly, triplex molecules can be provided targeted to the corresponding DNA target regions, and regions containing the DNA equivalent of a target sequence or a sequence complementary to the specified target (substrate) sequence. Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both.

By “consists essentially of” is meant that the active nucleic acid molecule of the invention, for example, an enzymatic nucleic acid molecule, contains an enzymatic center or core equivalent to those in the examples, and binding arms able to bind RNA such that cleavage at the target site occurs. Other sequences can be present which do not interfere with such cleavage. Thus, a core region can, for example, include one or more loops, stem-loop structure, or linker which does not prevent enzymatic activity. Thus, the underlined regions in the sequences in Tables V and VI can be such a loop, stem-loop, nucleotide linker, and/or non-nucleotide linker and can be represented generally as sequence “X”. For example, a core sequence for a hammerhead enzymatic nucleic acid can comprise a conserved sequence, such as 5′-CUGAUGAG-3′ and 5′-CGAA-3′ connected by “X”, where X is 5′-GCCGUUAGGC-3′ (SEQ ID NO. 16201), or any other Stem II region known in the art, or a nucleotide and/or non-nucleotide linker. Similarly, for other nucleic acid molecules of the instant invention, such as Inozyme, G-cleaver, amberzyme, zinzyme, DNAzyme, antisense, 2-5A antisense, triplex forming nucleic acid, and decoy nucleic acids, other sequences or non-nucleotide linkers can be present that do not interfere with the function of the nucleic acid molecule.

In another aspect of the invention, enzymatic nucleic acids or antisense molecules that interact with target RNA molecules and inhibit HBV (specifically HBV RNA) activity are expressed from transcription units inserted into DNA or RNA vectors. The recombinant vectors are preferably DNA plasmids or viral vectors. Enzymatic nucleic acid or antisense expressing viral vectors can be constructed based on, but not limited to, adeno-associated virus, retrovirus, adenovirus, or alphavirus. Preferably, the recombinant vectors capable of expressing the enzymatic nucleic acids or antisense are delivered as described above, and persist in target cells. Alternatively, viral vectors can be used that provide for transient expression of enzymatic nucleic acids or antisense. Such vectors can be repeatedly administered as necessary. Once expressed, the enzymatic nucleic acids or antisense bind to the target RNA and inhibit its function or expression. Delivery of enzymatic nucleic acids or antisense expressing vectors can be systemic, such as by intravenous or intramuscular administration, by administration to target cells ex-planted from the patient followed by reintroduction into the patient, or by any other means that allow for introduction into the desired target cell. Antisense DNA can be expressed via the use of a single stranded DNA intracellular expression vector.

In another embodiment, the invention features nucleic acid-based inhibitors (e.g., enzymatic nucleic acid molecules (ribozymes), antisense nucleic acids, triplex DNA, decoys, aptamers, siRNA, antisense nucleic acids containing RNA cleaving chemical groups) and methods for their use to down regulate or inhibit the expression of RNA (e.g., HBV) capable of progression and/or maintenance of liver disease and failure.

In another embodiment, the invention features nucleic acid-based techniques (e.g., enzymatic nucleic acid molecules (ribozymes), antisense nucleic acids, triplex DNA, decoys, aptamers, siRNA, antisense nucleic acids containing RNA cleaving chemical groups) and methods for their use to down regulate or inhibit the expression of HBV RNA expression.

In other embodiments, the invention features a method for the analysis of HBV proteins. This method is useful in determining the efficacy of HBV inhibitors. Specifically, the instant invention features an assay for the analysis of HBsAg proteins and secreted alkaline phosphatase (SEAP) control proteins to determine the efficacy of agents used to modulate HBV expression.

The method consists of coating a micro-titer plate with an antibody such as anti-HBsAg Mab (for example, Biostride B88-95-31ad,ay) at 0.1 to 10 μg/ml in a buffer (for example, carbonate buffer, such as Na₂CO₃ 15 mM, NaHCO₃ 35 mM, pH 9.5) at 4° C. overnight. The microtiter wells are then washed with PBST or the equivalent thereof, (for example, PBS, 0.05% Tween 20) and blocked for 0.1-24 hr at 37° C. with PBST, 1% BSA or the equivalent thereof. Following washing as above, the wells are dried (for example, at 37° C. for 30 min). Biotinylated goat anti-HBsAg or an equivalent antibody (for example, Accurate YVS1807) is diluted (for example at 1:1000) in PBST and incubated in the wells (for example, 1 hr. at 37° C.). The wells are washed with PBST (for example, 4×). A conjugate, (for example, Streptavidin/Alkaline Phosphatase Conjugate, Pierce 21324) is diluted to 10-10,000 ng/ml in PBST, and incubated in the wells (for example, 1 hr. at 37° C.). After washing as above, a substrate (for example, p-nitrophenyl phosphate substrate, Pierce 37620) is added to the wells, which are then incubated (for example, 1 hr. at 37° C.). The optical density is then determined (for example, at 405 nm). SEAP levels are then assayed, for example, using the Great EscAPe® Detection Kit (Clontech K2041-1), as per the manufacturers instructions. In the above example, incubation times and reagent concentrations can be varied to achieve optimum results, a non-limiting example is described in Example 6.

Comparison of this HBsAg ELISA method to a commercially available assay from World Diagnostics, Inc. 15271 NW 60^{th} Ave, #201, Miami Lakes, Fla. 33014 (305) 827-3304 (Cat. No. EL10018) demonstrates an increase in sensitivity (signal:noise) of 3-20 fold.

This invention also relates to nucleic acid molecules directed to disrupt the function of HBV reverse transcriptase. In addition, the invention relates to nucleic acid molecules directed to disrupt the function of the Enhancer I core region of the HBV genomic DNA. In particular, the present invention describes the selection and function of nucleic acid molecules, such as decoys and aptamers, capable of specifically binding to the HBV reverse transcriptase (pol) primer and modulating reverse transcription of the HBV pregenomic RNA. In another embodiment, the present invention relates to nucleic acid molecules, such as decoys, antisense and aptamers, capable of specifically binding to the HBV reverse transcriptase (pol) and modulating reverse transcription of the HBV pregenomic RNA. In yet another embodiment, the present invention relates to nucleic acid molecules capable of specifically binding to the HBV Enhancer I core region and modulating transcription of the HBV genomic DNA. The invention further relates to allosteric enzymatic nucleic acid molecules or “allozymes” that are used to modulate HBV gene expression. Such allozymes are active in the presence of HBV-derived nucleic acids, peptides, and/or proteins such as HBV reverse transcriptase and/or a HBV reverse transcriptase primer sequence, thereby allowing the allozyme to selectively cleave a sequence of HBV DNA or RNA. Allozymes of the invention are also designed to be active in the presence of HBV Enhancer I sequences and/or mutant HBV Enhancer I sequences, thereby allowing the allozyme to selectively cleave a sequence of HBV DNA or RNA. These nucleic acid molecules can be used to treat diseases and disorders associated with HBV infection.

In one embodiment, the invention features a nucleic acid decoy molecule that specifically binds the hepatitis B virus (HBV) reverse transcriptase primer sequence. In another embodiment, the invention features a nucleic acid decoy molecule that specifically binds the hepatitis B virus (HBV) reverse transcriptase. In yet another embodiment, the invention features a nucleic acid decoy molecule that specifically binds to the HBV Enhancer I core sequence.

In one embodiment, the invention features a nucleic acid aptamer that specifically binds the hepatitis B virus (HBV) reverse transcriptase primer. In another embodiment, the invention features a nucleic acid aptamer that specifically binds the hepatitis B virus (HBV) reverse transcriptase. In yet another embodiment, the invention features a nucleic acid aptamer molecule that specifically binds to the HBV Enhancer I core sequence.

In one embodiment, the invention features an allozyme that specifically binds the hepatitis B virus (HBV) reverse transcriptase primer. In another embodiment, the invention features an allozyme that specifically binds the hepatitis B virus (HBV) reverse transcriptase. In yet another embodiment, the invention features an allozyme that specifically binds to the HBV Enhancer I core sequence.

In yet another embodiment, the invention features a nucleic acid molecule, for example a triplex forming nucleic acid molecule or antisense nucleic acid molecule, that binds the hepatitis B virus (HBV) reverse transcriptase primer. In another embodiment, the invention features a triplex forming nucleic acid molecule or antisense nucleic acid molecule that specifically binds the hepatitis B virus (HBV) reverse transcriptase. In yet another embodiment, the invention features a triplex forming nucleic acid molecule or antisense nucleic acid molecule that specifically binds to the HBV Enhancer I core sequence.

In another embodiment, a nucleic acid molecule of the invention binds to Hepatocyte Nuclear Factor 3 (HNF3) and/or Hepatocyte Nuclear Factor 4 (HNF4) binding sequence within the HBV Enhancer I region of HBV genomic DNA, for example the plus strand and/or minus strand DNA of the Enhancer I region, and blocks the binding of HNF3 and/or HNF4 to the Enhancer 1 region.

In another embodiment, the nucleic acid molecule of the invention comprises a sequence having (UUCA)ₙ domain, where n is an integer from 1-10. In another embodiment, the nucleic acid molecules of the invention comprise the sequence of SEQ. ID NOs: 11216-11342.

In another embodiment, the invention features a composition comprising a nucleic acid molecule of the invention and a pharmaceutically acceptable carrier. In another embodiment, the invention features a mammalian cell, for example a human cell, including a nucleic acid molecule contemplated by the invention.

In one embodiment, the invention features a method for treatment of HBV infection, cirrhosis, liver failure, or hepatocellular carcinoma, comprising administering to a patient a nucleic acid molecule of the invention under conditions suitable for the treatment.

In another embodiment, the invention features a method for the treatment of a patient having a condition associated with HBV infection comprising contacting cells of said patient with a nucleic acid molecule of the invention under conditions suitable for such treatment. In another embodiment, the invention features a method for the treatment of a patient having a condition associated with HBV infection comprising contacting cells of said patient with a nucleic acid molecule of the invention, and further comprising the use of one or more drug therapies, for example type I interferon or 3TC® (lamivudine), under conditions suitable for said treatment. In another embodiment, the other therapy is administered simultaneously with or separately from the nucleic acid molecule.

In another embodiment, the invention features a method for modulating HBV replication in a mammalian cell comprising administering to the cell a nucleic acid molecule of the invention under conditions suitable for the modulation.

In yet another embodiment, the invention features a method of modulating HBV reverse transcriptase activity comprising contacting a nucleic acid molecule of the invention, for example a decoy or aptamer, with HBV reverse transcriptase under conditions suitable for the modulating of the HBV reverse transcriptase activity.

In another embodiment, the invention features a method of modulating HBV transcription comprising contacting a nucleic molecule of the invention with a HBV Enhancer I sequence under conditions suitable for the modulation of HBV transcription.

In one embodiment, a nucleic acid molecule of the invention, for example a decoy or aptamer, is chemically synthesized. In another embodiment, the nucleic acid molecule of the invention comprises at least one nucleic acid sugar modification. In yet another embodiment, the nucleic acid molecule of the invention comprises at least one nucleic acid base modification. In another embodiment, the nucleic acid molecule of the invention comprises at least one nucleic acid backbone modification.

In another embodiment, the nucleic acid molecule of the invention comprises at least one 2′-O-alkyl, 2′-alkyl, 2′-alkoxylalkyl, 2′-alkylthioalkyl, 2′-amino, 2′-O-amino, or 2′-halo modification and/or any combination thereof with or without 2′-deoxy and/or 2′-ribo nucleotides. In yet another embodiment, the nucleic acid molecule of the invention comprises all 2′-O-alkyl nucleotides, for example, all 2′-O-allyl nucleotides.

In one embodiment, the nucleic acid molecule of the invention comprises a 5′-cap, 3′-cap, or 5′-3′ cap structure, for example an abasic or inverted abasic moiety.

In another embodiment, the nucleic acid molecule of the invention is a linear nucleic acid molecule. In another embodiment, the nucleic acid molecule of the invention is a linear nucleic acid molecule that can optionally form a hairpin, loop, stem-loop, or other secondary structure. In yet another embodiment, the nucleic acid molecule of the invention is a circular nucleic acid molecule.

In one embodiment, the nucleic acid molecule of the invention is a single stranded oligonucleotide. In another embodiment, the nucleic acid molecule of the invention is a double-stranded oligonucleotide.

In one embodiment, the nucleic acid molecule of the invention comprises an oligonucleotide having between about 3 and about 100 nucleotides. In another embodiment, the nucleic acid molecule of the invention comprises an oligonucleotide having between about 3 and about 24 nucleotides. In another embodiment, the nucleic acid molecule of the invention comprises an oligonucleotide having between about 4 and about 16 nucleotides.

The nucleic acid decoy molecules and/or aptamers that bind to a reverse transcriptase and/or reverse transcriptase primer and therefore inactivate the reverse transcriptase, represent a novel therapeutic approach to treat a variety of pathologic indications, including, viral infection such as HBV infection, hepatitis, hepatocellular carcinoma, tumorigenesis, cirrhosis, liver failure and others.

The nucleic acid molecules that bind to a HBV Enhancer I sequence and therefore inactivate HBV transcription, represent a novel therapeutic approach to treat a variety of pathologic indications, including viral infection such as HBV infection, hepatitis, hepatocellular carcinoma, tumorigenesis, cirrhosis, liver failure and others conditions associated with the level of HBV.

In one embodiment of the present invention, a decoy nucleic acid molecule of the invention is 4 to 50 nucleotides in length, in specific embodiments about 4, 5, 6, 7, 8, 9, 10, 11, 12, 13, 14, 15, or 16 nucleotides in length. In another embodiment, a non-decoy nucleic acid molecule, e.g., an antisense molecule, a triplex DNA, or a ribozyme, is 13 to 100 nucleotides in length, e.g., in specific embodiments 35, 36, 37, or 38 nucleotides in length (e.g., for particular ribozymes or antisense). In particular embodiments, the nucleic acid molecule is 15-100, 17-100, 20-100, 21-100, 23-100, 25-100, 27-100, 30-100, 32-100, 35-100, 40-100, 50-100, 60-100, 70-100, or 80-100 nucleotides in length. Instead of 100 nucleotides being the upper limit on the length ranges specified above, the upper limit of the length range can be, for example, 30, 40, 50, 60, 70, or 80 nucleotides. Thus, for any of the length ranges, the length range for particular embodiments has lower limit as specified, with an upper limit as specified which is greater than the lower limit. For example, in a particular embodiment, the length range can be 35-50 nucleotides in length. All such ranges are expressly included. Also in particular embodiments, a nucleic acid molecule can have a length which is any of the lengths specified above, for example, 21 nucleotides in length.

Exemplary nucleic acid decoy molecules of the invention are shown in Table XIV. Exemplary synthetic nucleic acid molecules of the invention are shown in Table XV. For example, decoy molecules of the invention are between 4 and 40 nucleotides in length. Exemplary decoys of the invention are 4, 8, 12, or 16 nucleotides in length. In an additional example, enzymatic nucleic acid molecules of the invention are preferably between 15 and 50 nucleotides in length, more preferably between 25 and 40 nucleotides in length, e.g., 34, 36, or 38 nucleotides in length (for example see Jarvis et al., 1996, J. Biol. Chem., 271, 29107-29112). Exemplary DNAzymes of the invention are preferably between 15 and 40 nucleotides in length, more preferably between 25 and 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Exemplary antisense molecules of the invention are preferably between 15 and 75 nucleotides in length, more preferably between 20 and 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). Exemplary triplex forming oligonucleotide molecules of the invention are preferably between 10 and 40 nucleotides in length, more preferably between 12 and 25 nucleotides in length, e.g., 18, 19, 20, or 21 nucleotides in length (see for example Maher et al., 1990, Biochemistry, 29, 8820-8826; Strobel and Dervan, 1990, Science, 249, 73-75). Those skilled in the art will recognize that all that is required is that the nucleic acid molecule is of length and conformation sufficient and suitable for the nucleic acid molecule to catalyze a reaction contemplated herein. The length of the nucleic acid molecules of the instant invention are not limiting within the general limits stated.

In one embodiment, the invention provides a method for producing a class of nucleic acid-based gene modulating agents, which exhibit a high degree of specificity for a viral reverse transcriptase such as HBV reverse transcriptase or reverse transcriptase primer such as a HBV reverse transcriptase primer. For example, the nucleic acid molecule is preferably targeted to a highly conserved nucleic acid binding region of the viral reverse transcriptase such that specific treatment of a disease or condition can be provided with either one or several nucleic acid molecules of the invention. Such nucleic acid molecules can be delivered exogenously to specific tissue or cellular targets as required. Alternatively, the nucleic acid molecules can be expressed from DNA and/or RNA vectors that are delivered to specific cells.

In another embodiment, the invention provides a method for producing a class of nucleic acid-based gene modulating agents which exhibit a high degree of specificity for a viral enhancer regions such as the HBV Enhancer I core sequence. For example, the nucleic acid molecule is preferably targeted to a highly conserved transcription factor-binding region of the viral Enhancer I sequence such that specific treatment of a disease or condition can be provided with either one or several nucleic acid molecules of the invention. Such nucleic acid molecules can be delivered exogenously to specific tissue or cellular targets as required. Alternatively, the nucleic acid molecules can be expressed from DNA and/or RNA vectors that are delivered to specific cells.

In a another embodiment the invention provides a method for producing a class of enzymatic cleaving agents which exhibit a high degree of specificity for the RNA of a desired target. The enzymatic nucleic acid molecule, nuclease activating compound or chimera is preferably targeted to a highly conserved sequence region of a target mRNAs encoding HCV or HBV proteins such that specific treatment of a disease or condition can be provided with either one or several enzymatic nucleic acids. Such nucleic acid molecules can be delivered exogenously to specific cells as required. Alternatively, the enzymatic nucleic acid molecules can be expressed from DNA/RNA vectors that are delivered to specific cells. DNAzymes can be synthesized chemically or expressed endogenously in vivo, by means of a single stranded DNA vector or equivalent thereof.

In another embodiment, the nucleic acid molecule of the invention binds irreversibly to the HBV reverse transcriptase target, for example by covalent attachment of the nucleic molecule to the reverse transcriptase primer sequence. The covalent attachment can be accomplished by introducing chemical modifications into the nucleic acid molecule's (for example, decoy or aptamer) sequence that are capable of forming covalent bonds to the reverse transcriptase primer sequence.

In another embodiment, the nucleic acid molecule of the invention binds irreversibly to the HBV Enhancer I sequence target, for example, by covalent attachment of the nucleic acid molecule to the HBV Enhancer I sequence. The covalent attachment can be accomplished by introducing chemical modifications into the nucleic acid molecule's sequence that are capable of forming covalent bonds to the reverse transcriptase primer sequence.

In another embodiment, the type I interferon contemplated by the invention is interferon alpha, interferon beta, consensus interferon, polyethylene glycol interferon, polyethylene glycol interferon alpha 2a, polyethylene glycol interferon alpha 2b, polyethylene glycol consensus interferon.

In one embodiment, the invention features a composition comprising type I interferon and a nucleic acid molecule of the invention and a pharmaceutically acceptable carrier.

In another embodiment, the invention features a method of administering to a cell, for example a mammalian cell or human cell, a nucleic acid molecule of the invention independently or in conjunction with other therapeutic compounds, such as type I interferon or 3TC® (lamivudine), comprising contacting the cell with the nucleic acid molecule under conditions suitable for the administration.

In yet another embodiment, the invention features a method of administering to a cell, for example a mammalian cell or human cell, a nucleic acid molecule of the invention independently or in conjunction with other therapeutic compounds such as enzymatic nucleic acid molecules, antisense molecules, triplex forming oligonucleotides, 2,5-A chimeras, and/or RNAi, comprising contacting the cell with the nucleic acid molecule of the invention under conditions suitable for the administration.

In another embodiment, administration of a nucleic acid molecule of the invention is administered to a cell or patient in the presence of a delivery reagent, for example a lipid, cationic lipid, phospholipid, or liposome.

In one embodiment, the invention features novel nucleic acid-based techniques such as nucleic acid decoy molecules and/or aptamers, used alone or in combination with enzymatic nucleic acid molecules, antisense molecules, and/or RNAi, and methods for use to down regulate or modulate the expression of HBV RNA and/or replication of HBV.

In another embodiment, the invention features the use of one or more of the nucleic acid-based techniques to modulate the expression of the genes encoding HBV viral proteins. Specifically, the invention features the use of nucleic acid-based techniques to specifically modulate the expression of the HBV viral genome.

In another embodiment, the invention features the use of one or more of the nucleic acid-based techniques to modulate the activity, expression, or level of cellular proteins required for HBV replication. For example, the invention features the use of nucleic acid-based techniques to specifically modulate the activity of cellular proteins required for HBV replication.

In another embodiment, the invention features nucleic acid-based modulators (e.g., nucleic acid decoy molecules, aptamers, enzymatic nucleic acid molecules (ribozymes), antisense nucleic acids, triplex DNA, antisense nucleic acids containing RNA cleaving chemical groups) and methods for their use to down regulate or modulate reverse transcriptase activity and/or the expression of RNA (e.g., HBV) capable of progression and/or maintenance of HBV infection, hepatocellular carcinoma, liver disease and failure.

In another embodiment, the invention features nucleic acid-based techniques (e.g., nucleic acid decoy molecules, aptamers, enzymatic nuleic acid molecules (ribozymes), antisense nucleic acid molecules, triplex DNA, antisense nucleic acids containing RNA cleaving chemical groups) and methods for their use to down regulate or modulate reverse transcriptase activity and/or the expression of HBV RNA.

In another embodiment, the invention features nucleic acid-based modulators (e.g., nucleic acid decoy molecules, aptamers, enzymatic nucleic acid molecules (ribozymes), antisense nucleic acids, triplex DNA, siRNA, dsRNA, antisense nucleic acids containing RNA cleaving chemical groups) and methods for their use to down regulate or modulate Enhancer I mediated transcription activity and/or the expression of DNA (e.g., HBV) capable of progression and/or maintenance of HBV infection, hepatocellular carcinoma, liver disease and failure.

In another embodiment, the invention features nucleic acid-based techniques (e.g., nucleic acid decoy molecules, aptamers, enzymatic nucleic acid molecules, antisense nucleic acid molecules, triplex DNA, siRNA, antisense nucleic acids containing DNA cleaving chemical groups) and methods for their use to down regulate or modulate Enhancer I mediated transcription activity and/or the expression of HBV DNA.

In another embodiment, the invention features a nucleic acid sensor molecule having an enzymatic nucleic acid domain and a sensor domain that interacts with an HBV peptide, protein, or polynucleotide sequence, for example, HBV reverse transcriptase, HBV reverse transcriptase primer, or the Enhancer I element of the HBV pregenomic RNA, wherein such interaction results in modulation of the activity of the enzymatic nucleic acid domain of the nucleic acid sensor molecule. In another embodiment, the invention features HBV-specific nucleic acid sensor molecules or allozymes, and methods for their use to down regulate or modulate the expression of HBV RNA capable of progression and/or maintenance of hepatitis, hepatocellular carcinoma, cirrhosis, and/or liver failure. In yet another embodiment, the enzymatic nucleic acid domain of a nucleic acid sensor molecule of the invention is a Hammerhead, Inozyme, G-cleaver, DNAzyme, Zinzyme, Amberzyme, or Hairpin enzymatic nucleic acid molecule.

In one embodiment, nucleic acid molecules of the invention are used to treat HBV-infected cells or a HBV-infected patient wherein the HBV is resistant or the patient does not respond to treatment with 3TC® (Lamivudine), either alone or in combination with other therapies under conditions suitable for the treatment.

In another embodiment, nucleic acid molecules of the invention are used to treat HBV-infected cells or a HBV-infected patient, wherein the HBV is resistant or the patient does not respond to treatment with Interferon, for example Infergen®, either alone or in combination with other therapies under conditions suitable for the treatment.

The invention also relates to in vitro and in vivo systems, including, e.g., mammalian systems for screening inhibitors of HBV. In one embodiment, the invention features a mouse, for example a male or female mouse, implanted with HepG2.2.15 cells, wherein the mouse is susceptible to HBV infection and capable of sustaining HBV DNA expression. One embodiment of the invention provides a mouse implanted with HepG2.2.15 cells, wherein said mouse sustains the propagation of HEPG2.2.15 cells and HBV production.

In another embodiment, a mouse of the invention has been infected with HBV for at least one week to at least eight weeks, including, for example at least 4 weeks.

In yet another embodiment, a mouse of the invention, for example a male or female mouse, is an immunocompromised mouse, for example a nu/nu mouse or a scid/scid mouse.

In one embodiment, the invention features a method of producing a mouse of the invention, comprising injecting, for example by subcutaneous injection, HepG2.2.15 (Sells, et al, 1987, Proc Natl Acad Sci USA., 84, 1005-1009) cells into the mouse under conditions suitable for the propagation of HepG2.2.15 cells in said mouse. HepG2.2.15 cells can be suspended in, for example, Delbecco's PBS solution including calcium and magnesium. In another embodiment, HepG2.2.15 cells are selected for antibiotic resistance and are then introduced into the mouse under conditions suitable for the propagation of HepG2.2.15 cells in said mouse. A non-limiting example of antibiotic resistant HepG2.2.15 cells include G418 antibiotic resistant HepG2.2.15 cells.

In another embodiment, the invention features a method of screening a compound for therapeutic activity against HBV, comprising administering the compound to a mouse of the invention and monitoring the the levels of HBV produced (e.g. by assaying for HBV DNA levels) in the mouse.

In one embodiment, a therapeutic compound or therapy contemplated by the invention is a lipid, steroid, peptide, protein, antibody, monoclonal antibody, humanized monoclonal antibody, small molecule, and/or isomers and analogs thereof, and/or a cell.

In one embodiment, a therapeutic compound or therapy contemplated by the invention is a nucleic acid molecule, for example a nucleic acid molecule, such as an enzymatic nucleic acid molecule, antisense nucleic acid molecule, allozyme, peptide nucleic acid, decoy, triplex oligonucleotide, dsRNA, ssRNA, RNAi, siRNA, aptamer, or 2,5-A chimera used alone or in combination with another therapy, for example antiviral therapy. Antiviral therapy can be, for example, treatment with 3TC® (Lamivudine) or interferon. Interferon can include, for example, consensus interferon or type I interferon. Type I interferon can include interferon alpha, interferon beta, consensus interferon, polyethylene glycol interferon, polyethylene glycol interferon alpha 2a, polyethylene glycol interferon alpha 2b, or polyethylene glycol consensus interferon.

In one embodiment, the invention features a non-human mammal implanted with HepG2.2.15 cells, wherein the non-human mammal is susceptible to HBV infection and capable of sustaining HBV DNA expression in the implanted HepG2.2.15 cells.

In another embodiment, a non-human mammal of the invention, for example a male or female non-human mammal, has been infected with HBV for at least one week to at least eight weeks, including for example at least four weeks.

In yet another embodiment, a non-human mammal of the invention is an immunocompromised mammal, for example a nu/nu mammal or a scid/scid mammal.

In one embodiment, the invention features a method of producing a non-human mammal comprising HepG2.2.15 cells comprising injecting, for example by subcutaneous injection, HepG2.2.15 cells into the non-human mammal under conditions suitable for the propagation of HepG2.2.15 cells in said non-human mammal.

In another embodiment, the invention features a method of screening a compound for therapeutic activity against HBV comprising administering the compound to a non-human mammal of the invention and monitoring the levels of HBV produced (e.g. by assaying for HBV DNA levels) in the non-human mammals.

In one embodiment, a therapeutic compound or therapy contemplated by the invention is a nucleic acid molecule, for example an enzymatic nucleic acid molecule, allozyme, antisense nucleic acid molecule, decoy, triplex oligonucleotide, dsRNA, ssRNA, RNAi, siRNA, or 2,5-A chimera used alone or in combination with another therapy, for example antiviral therapy.

Methods and chimeric immunocompromised heterologous non-human mammalian hosts, particularly mouse hosts, are provided for the expression of hepatitis B virus (“HBV”). In one embodiment, the chimeric hosts have transplanted viable, HepG2.2.15 cells in an immunocompromised host.

The non-human mammals contemplated by the invention are immunocompromised in normally inheriting the desired immune incapacity, or the desired immune incapacity can be created. For example, hosts with severe combined immunodeficiency, known as scid/scid hosts, are available. Rodentia, particularly mice, and equine, particularly horses, are presently available as scid/scid hosts, for example scid/scid mice and scid/scid rats. The scid/scid hosts lack functioning lymphocyte types, particularly B-cells and some T-cell types. In the scid/scid mouse hosts, the genetic defect appears to be a non-functioning recombinase, as the germline DNA is not rearranged to produce functioning surface immunoglobulin and T-cell receptors.

Any immunodeficient non-human mammals, e.g. mouse, can be used to generate the animal models described herein. The term “immunodeficient,” as used herein, refers to a genetic alteration that impairs the animal's ability to mount an effective immune response. In this regard, an “effective immune response” is one which is capable of destroying invading pathogens such as (but not limited to) viruses, bacteria, parasites, malignant cells, and/or a xenogeneic or allogeneic transplant. In one embodiment, the immunodeficient mouse is a severe immunodeficient (SCID) mouse, which lacks recombinase activity that is necessary for the generation of immunoglobulin and functional T cell antigen receptors, and thus does not produce functional B and T lymphocytes. In another embodiment, the immunodeficient mouse is a nude mouse, which contains a genetic defect that results in the absence of a functional thymus, leading to T-cell and B-cell deficiencies. However, mice containing other immunodeficiencies (such as rag-1 or rag-2 knockouts, as described in Chen et al., 1994, Curr. Opin. Immunol, 6, 313-319 and Guidas et al., 1995, J. Exp. Med., 181, 1187-1195, or beige-nude mice, which also lack natural killer cells, as described in Kollmann et al., 1993, J. Exp. Med., 177, 821-832) can also be employed.

The introduction of HepG2.2.15 cells occurs with a host at an age less than about 25% of its normal lifespan, usually to 20% of the normal lifespan with mice, and the age will generally be of an age of about 3 to 10 weeks, more usually from about 4 to 8 weeks. The mice can be of either sex, can be neutered, and can be otherwise normal, except for the immunocompromised state, or they can have one or more mutations, which can be naturally occurring or as a result of mutagenesis.

In another embodiment, the mouse model described herein is used to evaluate the effectiveness of the therapeutic compounds and methods. The terms “therapeutic compounds”, “therapeutic methods” and “therapy” as used herein, encompass exogenous factors, such as dietary or environmental conditions, as well as pharmaceutical compositions “drugs” and vaccines. In one embodiment, the therapeutic method is an immunotherapy, which can include the treatment of the HBV bearing animal with populations of HBV-reactive immune cells. The therapeutic method can also, or alternatively, be a gene therapy (i.e., a therapy that involves treatment of the HBV-bearing mouse with a cell population that has been manipulated to express one or more genes, the products of which can possess anti-viral activity), see for example The Development of Human Gene Therapy, Theodore Friedmann, Ed. Cold Spring Harbor Laboratory Press, Cold Spring Harbor, N.Y., 1999. Therapeutic compounds of the invention can comprise a drug or composition with pharmaceutical activity that can be used to treat illness or disease. A therapeutic method can comprise the use of a plurality of compounds in a mixture or a distinct entity. Examples of such compounds include nucleosides, nucleic acids, nucleic acid chimeras, RNA and DNA oligonucleotides, peptide nucleic acids, enzymatic nucleic acid molecules, antisense nucleic acid molecules, decoys, triplex oligonucleotides, ssDNA, dsRNA, ssRNA, siRNA, 2,5-A chimeras, lipids, steroids, peptides, proteins, antibodies, monoclonal antibodies (see for example Hall, 1995, Science, 270, 915-916), small molecules, and/or isomers and analogs thereof.

The methods of this invention can be used to treat human hepatitis B virus infections, which include productive virus infection, latent or persistent virus infection, and HBV-induced hepatocyte transformation. The utility can be extended to other species of HBV that infect non-human animals where such infections are of veterinary importance.

Preferred binding sites of the nucleic acid molecules of the invention include, but are not limited, to the primer binding site on HBV reverse transcriptase, the primer binding sequences of the HBV RNA, and/or the HBV Enhancer I region of HBV DNA.

This invention further relates to nucleic acid molecules that target RNA species of hepatitis C virus (HCV) and/or encoded by the HCV. In one embodiment, applicant describes enzymatic nucleic acid molecules that specifically cleave HCV RNA and the selection and function thereof. The invention further relates to compounds and chimeric molecules comprising nuclease activating activity. The invention also relates to compositions and methods for the cleavage of RNA using these nuclease activating compounds and chimeras. Nucleic acid molecules, nuclease activating compounds and chimeras, and compostions and methods of the invention can be used to treat diseases associated with HCV infection.

Due to the high sequence variability of the HCV genome, selection of nucleic acid molecules and nuclease activating compounds and chimeras for broad therapeutic applications preferably involve the conserved regions of the HCV genome. Thus, in one embodiment the present invention describes nucleic acid molecules that cleave the conserved regions of the HCV genome. The invention further describes compounds and chimeric molecules that activate cellular nucleases that cleave HCV RNA, including concerved regions of the HCV genome. Examples of conserved regions of the HCV genome include but are not limited to the 5′-Non Coding Region (NCR), the 5′-end of the core protein coding region, and the 3′-NCR. HCV genomic RNA contains an internal ribosome entry site (IRES) in the 5′-NCR which mediates translation independently of a 5′-cap structure (Wang et al., 1993, J. Virol., 67, 3338-44). The full-length sequence of the HCV RNA genome is heterologous among clinically isolated subtypes, of which there are at least 15 (Simmonds, 1995, Hepatology, 21, 570-583), however, the 5′-NCR sequence of HCV is highly conserved across all known subtypes, most likely to preserve the shared IRES mechanism (Okamoto et al., 1991, J. General Virol., 72, 2697-2704). In general, enzymatic nucleic acid molecules and nuclease activating compounds, and chimeras that cleave sites located in the 5′ end of the HCV genome are expected to block translation while nucleic acid molecules and nuclease activating compounds, and chimeras that cleave sites located in the 3′ end of the genome are expected to block RNA replication. Therefore, one nucleic acid molecule, compound, or chimera can be designed to cleave all the different isolates of HCV. Enzymatic nucleic acid molecules and nuclease activating compounds, and chimeras designed against conserved regions of various HCV isolates enable efficient inhibition of HCV replication in diverse patient populations and ensure the effectiveness of the nucleic acid molecules and nuclease activating compounds, and chimeras against HCV quasi species which evolve due to mutations in the non-conserved regions of the HCV genome.

In one embodiment, the invention features an enzymatic nucleic acid molecule, preferably in the hammerhead, NCH (Inozyme), G-cleaver, amberzyme, zinzyme and/or DNAzyme motif, and the use thereof to down-regulate or inhibit the expression of HCV RNA.

In another embodiment, the invention features an enzymatic nucleic acid molecule, preferably in the hammerhead, Inozyme, G-cleaver, amberzyme, zinzyme and/or DNAzyme motif, and the use thereof to down-regulate or inhibit the expression of HCV minus strand RNA.

In yet another embodiment, the invention featues a nuclease activating compound and/or a chimera and the use thereof to down-regulate or inhibit the expression of HCV RNA.

In another embodiment, the invention featues the use of a nuclease activating compound and/or a chimera to inhibit the expression of HCV minus strand RNA.

In one embodiment, the invention features a compound having formula I:

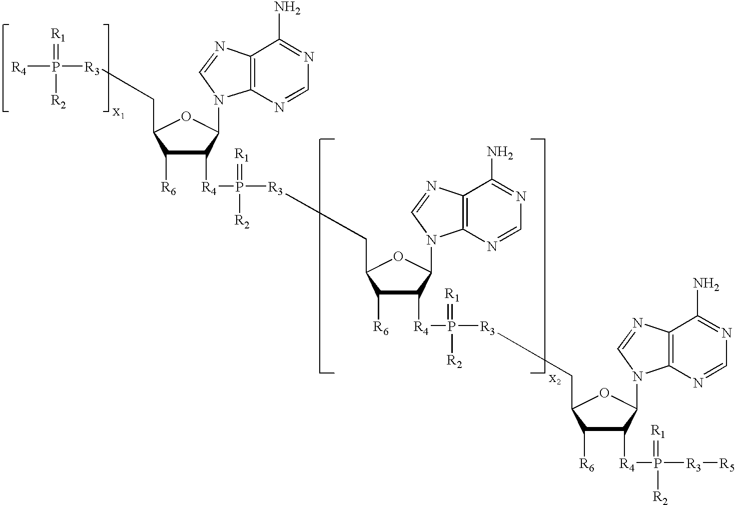

wherein X₁ is an integer selected from the group consisting of 1, 2, and 3; X₂ is an integer greater than or equal to 1; R₆ is independently selected from the group including H. OH, NH₂, ONH₂, alkyl, S-alkyl, O-alkyl, O-alkyl-S-alkyl, O-alkoxyalkyl, allyl, O-allyl, and fluoro; each R₁ and R₂ are independently selected from the group consisting of O and S; each R₃ and R₄ are independently selected from the group consisting of O, N, and S; and R₅ is selected from the group consisting of alkyl, alkylamine, an oligonucleotide having any of SEQ ID NOS. 11343-16182, an oligonucleotide having a sequence complementary to a sequence selected from the group including SEQ ID NOS. 2594-7433, and abasic moiety.

In another embodiment, the abasic moiety of the instant invention is selected from the group consisting of:

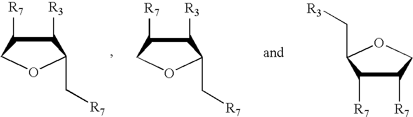

wherein R₃ is selected from the group consisting of O, N, and S, and R₇ is independently selected from the group consisting of H, OH, NH2, O—NH2, alkyl, S-alkyl, O-alkyl, O-alkyl-S-alkyl, O-alkoxyalkyl, allyl, O-allyl, fluoro, oligonucleotide, alkyl, alkylamine and abasic moiety.

In another embodiment, the oligonucleotide R₅ of Formula I having a sequence complementary to a sequence selected from the group consisting of SEQ ID NOS. 2594-7433 is an enzymatic nucleic acid molecule.

In yet another embodiment, the oligonucleotide R₅ of Formula I having a sequence complementary to a sequence selected from the group consisting of SEQ ID NOS. 2594-7433 is an antisense nucleic acid molecule.

In another embodiment, the oligonucleotide R₅ of Formula I having a sequence complementary to a sequence selected from the group consisting of SEQ ID NOS. 2594-7433 is an enzymatic nucleic acid molecule selected from the group consisting of Hammerhead, Inozyme, G-cleaver, DNAzyme, Amberzyme, and Zinzyme motifs.

In another embodiment, the Inozyme enzymatic nucleic acid molecule of the instant invention comprises a stem II region of length greater than or equal to 2 base pairs.

In one embodiment, the oligonucleotide R₅ of Formula I having a sequence complementary to a sequence selected from the group consisting of SEQ ID NOS. 2594-7433 is an enzymatic nucleic acid comprising between 12 and 100 bases complementary to an RNA derived from HCV.

In another embodiment, the oligonucleotide R₅ of Formula I having a sequence complementary to a sequence selected from the group consisting of SEQ ID NOS. 2594-7433 is an enzymatic nucleic acid comprising between 14 and 24 bases complementary to said RNA derived from HCV.

In one embodiment, the oligonucleotide R₅ of Formula I having a sequence complementary to a sequence selected from the group consisting of SEQ ID NOS. 2594-7433 is an antisense nucleic acid comprising between 12 and 100 bases complementary to an RNA derived from HCV.

In another embodiment, the oligonucleotide R₅ of Formula I having a sequence complementary to a sequence selected from the group consisting of SEQ ID NOS. 2594-7433 is an antisense nucleic acid comprising between 14 and 24 bases complementary to said RNA derived from HCV.

In another embodiment, the invention features a composition comprising a compound of Formula I, in a pharmaceutically acceptable carrier.

In yet another embodiment, the invention features a mammalian cell comprising a compound of Formula I. For example, the mammalian cell comprising a compound of Formula I can be a human cell.

In one embodiment, the invention features a method for the treatment of cirrhosis, liver failure, hepatocellular carcinoma, or a condition associated with HCV infection comprising the step of administering to a patient a compound of Formula I under conditions suitable for said treatment.

In another embodiment, the invention features a method of treatment of a patient having a condition associated with HCV infection comprising contacting cells of said patient with a compound having Formula I, and further comprising the use of one or more drug therapies under conditions suitable for said treatment. For example, the other therapies of the instant invention can be selected from the group consisting of type I interferon, interferon alpha, interferon beta, consensus interferon, polyethylene glycol interferon, polyethylene glycol interferon alpha 2a, polyethylene glycol interferon alpha 2b, polyethylene glycol consensus interferon, treatment with an enzymatic nucleic acid molecule, and treatment with an antisense molecule.

In another embodiment, the other therapies of the instant invention, for example type I interferon, interferon alpha, interferon beta, consensus interferon, polyethylene glycol interferon, polyethylene glycol interferon alpha 2a, polyethylene glycol interferon alpha 2b, polyethylene glycol consensus interferon, treatment with an enzymatic nucleic acid molecule, and treatment with an antisense nucleic acid molecule, and the compound having Formula I are administered separately in separate pharmaceutically acceptable carriers.

In yet another embodiment, the other therapies of the instant invention, for example type I interferon, interferon alpha, interferon beta, consensus interferon, polyethylene glycol interferon, polyethylene glycol interferon alpha 2a, polyethylene glycol interferon alpha 2b, polyethylene glycol consensus interferon, treatment with an enzymatic nucleic acid molecule, and treatment with an antisense nucleic acid molecule, and the compound having Formula I are administered simultaneously in a pharmaceutically acceptable carrier. The invention features a composition comprising a compound of Formula I and one or more of the above-listed compounds in a pharmaceutically acceptable carrier.

In yet another embodiment, the invention features a method for inhibiting HCV replication in a mammalian cell comprising the step of administering to said cell a compound having Formula I under conditions suitable for said inhibition.

In another embodiment, the invention features a method of cleaving a separate RNA molecule (i.e., HCV RNA or RNA necessary for HCV replication) comprising contacting a compound having Formula I with the separate RNA molecule under conditions suitable for the cleavage of the separate RNA molecule. In one example, the method of cleaving a separate RNA molecule is carried out in the presence of a divalent cation, for example Mg2+.

In yet another embodiment, the method of cleaving a separate RNA molecule of the invention is carried out in the presence of a protein nuclease, for example RNAse L.

In one embodiment, a compound having Formula I is chemically synthesized. In one embodiment, a compound having Formula I comprises at least one 2′-sugar modification, at least one nucleic acid base modification, and/or at least one phosphate modification.

The nucleic acid-based modulators of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers. In particular embodiments, the nucleic acid molecules of the invention comprise sequences shown in Tables IV-XI, XIV-XV and XVIII-XXIII. Examples of such nucleic acid molecules consist essentially of sequences defined in the tables.

The nucleic acid-based inhibitors, nuclease activating compounds and chimeras of the invention are added directly, or can be complexed with cationic lipids, packaged within liposomes, or otherwise delivered to target cells or tissues. The nucleic acid or nucleic acid complexes, and nuclease activating compounds or chimeras can be locally administered to relevant tissues ex vivo, or in vivo through injection or infusion pump, with or without their incorporation in biopolymers. In preferred embodiments, the enzymatic nucleic acid inhibitors, and nuclease activating compounds or chimeras comprise sequences, which are complementary to the substrate sequences in Tables XVIII, XIX, XX and XXIII. Examples of such enzymatic nucleic acid molecules also are shown in Tables XVIII, XIX, XX, XXI and XXIII. Examples of such enzymatic nucleic acid molecules consist essentially of sequences defined in these tables. In additional embodiments, the enzymatic nucleic acid inhibitors of the invention that comprise sequences which are complementary to the substrate sequences in Tables XVIII, XIX, XX and XXIII are covalently attached to nuclease activating compound or chimeras of the invention, for example a compound having Formula I.

In yet another embodiment, the invention features antisense nucleic acid molecules and 2-5A chimera including sequences complementary to the substrate sequences shown in Tables XVIII, XIX, XX and XXIII. Such nucleic acid molecules can include sequences as shown for the binding arms of the enzymatic nucleic acid molecules in Tables XVIII, XIX, XX, XXI and XXIII. Similarly, triplex molecules can be provided targeted to the corresponding DNA target regions, and containing the DNA equivalent of a target sequence or a sequence complementary to the specified target (substrate) sequence. Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both.

In one embodiment, the invention features nucleic acid molecules and nuclease activating compounds or chimeras that inhibit gene expression and/or viral replication. These chemically or enzymatically synthesized nucleic acid molecules can contain substrate binding domains that bind to accessible regions of their target mRNAs. The nucleic acid molecules also contain domains that catalyze the cleavage of RNA. The enzymatic nucleic acid molecules are preferably molecules of the hammerhead, Inozyme, DNAzyme, Zinzyme, Amberzyme, and/or G-cleaver motifs. Upon binding, the enzymatic nucleic acid molecules cleave the target mRNAs, preventing translation and protein accumulation. In the absence of the expression of the target gene, HCV gene expression and/or replication is inhibited.

In another aspect, the invention provides mammalian cells containing one or more nucleic acid molecules and/or expression vectors of this invention. The one or more nucleic acid molecules can independently be targeted to the same or different sites.

In one embodiment, nucleic acid decoys, aptamers, siRNA, enzymatic nucleic acids or antisense molecules that interact with target protein and/or RNA molecules and modulate HBV (specifically HBV reverse transcriptase, or transcription of HBV genomic DNA) activity are expressed from transcription units inserted into DNA or RNA vectors. The recombinant vectors are preferably DNA plasmids or viral vectors. Decoys, aptamers, enzymatic nucleic acid or antisense expressing viral vectors can be constructed based on, but not limited to, adeno-associated virus, retrovirus, adenovirus, or alphavirus. Preferably, the recombinant vectors capable of expressing the decoys, aptamers, enzymatic nucleic acids or antisense are delivered as described above, and persist in target cells. Alternatively, viral vectors can be used that provide for transient expression of decoys, aptamers, siRNA, enzymatic nucleic acids or antisense. Such vectors can be repeatedly administered as necessary. Once expressed, the decoys, aptamers, enzymatic nucleic acids or antisense bind to the target protein and/or RNA and modulate its function or expression. Delivery of decoy, aptamer, siRNA, enzymatic nucleic acid or antisense expressing vectors can be systemic, such as by intravenous or intramuscular administration, by administration to target cells ex-planted from the patient followed by reintroduction into the patient, or by any other means that would allow for introduction into the desired target cell. DNA based nucleic acid molecules of the invention can be expressed via the use of a single stranded DNA intracellular expression vector.

In one embodiment, nucleic acid molecules and nuclease activating compounds or chimeras are added directly, or can be complexed with cationic lipids, packaged within liposomes, or otherwise delivered to target cells. The nucleic acid or nucleic acid complexes can be locally administered to relevant tissues ex vivo, or in vivo through injection, infusion pump or stent, with or without their incorporation in biopolymers. In another preferred embodiment, the nucleic acid molecule, nuclease activating compound or chimera is administered to the site of HBV or HCV activity (e.g., hepatocytes) in an appropriate liposomal vehicle.

In another embodiment, nucleic acid molecules that cleave target molecules and inhibit HCV activity are expressed from transcription units inserted into DNA or RNA vectors. The recombinant vectors are preferably DNA plasmids or viral vectors. Nucleic acid molecule expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecules cleave the target mRNA. Delivery of enzymatic nucleic acid molecule expressing vectors can be systemic, such as by intravenous or intramuscular administration, by administration to target cells ex-planted from the patient followed by reintroduction into the patient, or by any other means that would allow for introduction into the desired target cell (for a review see Couture and Stinchcomb, 1996, TIG., 12, 510). In another aspect of the invention, nucleic acid molecules that cleave target molecules and inhibit viral replication are expressed from transcription units inserted into DNA, RNA, or viral vectors. Preferably, the recombinant vectors capable of expressing the nucleic acid molecules are locally delivered as described above, and transiently persist in smooth muscle cells. However, other mammalian cell vectors that direct the expression of RNA can be used for this purpose.

The nucleic acid molecules of the instant invention, individually, or in combination or in conjunction with other drugs, and/or therapies can be used to treat diseases or conditions discussed herein. For example, to treat a disease or condition associated with the levels of HBV or HCV, the nucleic acid molecules can be administered to a patient or can be administered to other appropriate cells evident to those skilled in the art, individually or in combination with one or more drugs under conditions suitable for the treatment.

In a further embodiment, the described molecules, such as decoys, aptamers, antisense, enzymatic nucleic acids, or nuclease activating compounds and chimeras can be used in combination with other known treatments to treat conditions or diseases discussed above. For example, the described molecules could be used in combination with one or more known therapeutic agents to treat HBV infection, HCV infection, hepatitis, hepatocellular carcinoma, cancer, cirrhosis, and liver failure. Such therapeutic agents can include, but are not limited to, nucleoside analogs selected from the group comprising Lamivudine (3TC®), L-FMAU, and/or adefovir dipivoxil (for a review of applicable nucleoside analogs, see Colacino and Staschke, 1998, Progress in Drug Research, 50, 259-322). Immunomodulators selected from the group comprising Type 1 Interferon, therapeutic vaccines, steriods, and 2′-5′ oligoadenylates (for a review of 2′-5′ Oligoadenylates, see Charubala and Pfleiderer, 1994, Progress in Molecular and Subcellular Biology, 14, 113-138).

Nucleic acid molecules, nuclease activating compounds and chimeras of the invention, individually, or in combination or in conjunction with other drugs, can be used to treat diseases or conditions discussed above. For example, to treat a disease or condition associated with HBV or HCV levels, the patient can be treated, or other appropriate cells can be treated, as is evident to those skilled in the art.

In a further embodiment, the described molecules can be used in combination with other known treatments to treat conditions or diseases discussed above. For example, the described molecules can be used in combination with one or more known therapeutic agents to treat liver failure, hepatocellular carcinoma, cirrhosis, and/or other disease states associated with HBV or HCV infection. Additional known therapeutic agents are those comprising antivirals, interferons, and/or antisense compounds.

The term “inhibit” or “down-regulate” as used herein refers to the expression of the gene, or level of RNAs or equivalent RNAs encoding one or more protein subunits or components, or activity of one or more protein subunits or components, such as HBV protein or proteins, is reduced below that observed in the absence of the therapies of the invention. In one embodiment, inhibition or down-regulation with enzymatic nucleic acid molecule preferably is below that level observed in the presence of an enzymatically inactive or attenuated molecule that is able to bind to the same site on the target RNA, but is unable to cleave that RNA. In another embodiment, inhibition or down-regulation with antisense oligonucleotides is preferably below that level observed in the presence of, for example, an oligonucleotide with scrambled sequence or with mismatches. In another embodiment, inhibition or down-regulation of HBV with the nucleic acid molecule of the instant invention is greater in the presence of the nucleic acid molecule than in its absence.

The term “up-regulate” as used herein refers to the expression of the gene, or level of RNAs or equivalent RNAs encoding one or more protein subunits or components, or activity of one or more protein subunits or components, such as HBV or HCV protein or proteins, is greater than that observed in the absence of the therapies of the invention. For example, the expression of a gene, such as HBV or HCV genes, can be increased in order to treat, prevent, ameliorate, or modulate a pathological condition caused or exacerbated by an absence or low level of gene expression.

The term “modulate” as used herein refers to the expression of the gene, or level of RNAs or equivalent RNAs encoding one or more protein subunits or components, or activity of one or more proteins is up-regulated or down-regulated, such that the expression, level, or activity is greater than or less than that observed in the absence of the therapies of the invention.

The term “decoy” as used herein refers to a nucleic acid molecule, for example RNA or DNA, or aptamer that is designed to preferentially bind to a predetermined ligand. Such binding can result in the inhibition or activation of a target molecule. A decoy or aptamer can compete with a naturally occurring binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is but a specific example and those in the art will recognize that other embodiments can be readily generated using techniques generally known in the art, see for example Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628. Similarly, a decoy can be designed to bind to HBV or HCV proteins and block the binding of HBV or HCV DNA or RNA or a decoy can be designed to bind to HBV or HCV proteins and prevent molecular interaction with the HBV or HCV proteins.

By “aptamer” or “nucleic acid aptamer” as used herein is meant a nucleic acid molecule that binds specifically to a target molecule wherein the nucleic acid molecule has sequence that is distinct from sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art, see for example Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628.

By “enzymatic nucleic acid molecule” is meant a nucleic acid molecule that has complementarity in a substrate binding region to a specified gene target, and also has an enzymatic activity which is active to specifically cleave a target RNA molecule. That is, the enzymatic nucleic acid molecule is able to intermolecularly cleave a RNA molecule and thereby inactivate a target RNA molecule. These complementary regions allow sufficient hybridization of the enzymatic nucleic acid molecule to a target RNA molecule and thus permit cleavage. One hundred percent complementarity is preferred, but complementarity as low as 50-75% may also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids can be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it have a specific substrate binding site which is complementary to one or more of the target nucleic acid regions, and that it have nucleotide sequences within or surrounding that substrate binding site which impart a nucleic acid cleaving activity to the molecule (Cech et al., U.S. Pat. No. 4,987,071; Cech et al., 1988, JAMA 260:20 3030-4).

By “nucleic acid molecule” as used herein is meant a molecule comprising nucleotides. The nucleic acid can be single, double, or multiple stranded and can comprise modified or unmodified nucleotides or non-nucleotides or various mixtures and combinations thereof.

By “enzymatic portion” or “catalytic domain” is meant that portion/region of the enzymatic nucleic acid molecule essential for cleavage of a nucleic acid substrate (for example see FIGS. 1-5).

By “substrate binding arm” or “substrate binding domain” is meant that portion/region of a ribozyme which is complementary to (i.e., able to base-pair with) a portion of its substrate. Generally, such complementarity is 100%, but can be less if desired. For example, as few as 10 bases out of 14 may be base-paired (see for example Werner and Uhlenbeck, 1995, Nucleic Acids Research, 23, 2092-2096; Hammann et al., 1999, Antisense and Nucleic Acid Drug Dev., 9, 25-31). Such arms are shown generally in FIGS. 1-5. That is, these arms contain sequences within a ribozyme which are intended to bring ribozyme and target RNA together through complementary base-pairing interactions. The ribozyme of the invention can have binding arms that are contiguous or non-contiguous and may be of varying lengths. The length of the binding arm(s) are preferably greater than or equal to four nucleotides and of sufficient length to stably interact with the target RNA; specifically 12-100 nucleotides; more specifically 14-24 nucleotides long (see for example Werner and Uhlenbeck, supra; Hamman et al., supra; Hampel et al, EP0360257; Berzal-Herrance et al., 1993, EMBO J., 12, 2567-73). If two binding arms are chosen, the design is such that the length of the binding arms are symmetrical (i.e., each of the binding arms is of the same length; e.g., five and five nucleotides, six and six nucleotides or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “nuclease activating compound” is meant a compound, for example a compound having Formula I, that activates the cleavage of an RNA by a nuclease. The nuclease can comprise RNAse L. By “nuclease activating chimera” or “chimera” is meant a nuclease activating compound, for example a compound having Formula I, that is attached to a nulceic acid molecule, for example a nucleic acid molecule that binds preferentially to a target RNA. These chimeric nucleic acid molecules can comprise a nuclease activating compound and an antisense nucleic acid molecule, for example a 2′,5′-oligoadenylate antisense chimera, or an enzymatic nucleic acid moleucle, for example a 2′,5′-oligoadenylate enzymatic nucleic acid chimera.

By “Inozyme” or “NCH” motif or configuration is meant, an enzymatic nucleic acid molecule comprising a motif as is generally described as NCH Rz in Ludwig et al., International PCT Publication No. WO 98/58058 and U.S. patent application Ser. No. 08/878,640. Inozymes possess endonuclease activity to cleave RNA substrates having a cleavage triplet NCH/, where N is a nucleotide, C is cytidine and H is adenosine, uridine or cytidine, and/represents the cleavage site. Inozymes can also possess endonuclease activity to cleave RNA substrates having a cleavage triplet NCN/, where N is a nucleotide, C is cytidine, and/represents the cleavage site.

By “G-cleaver” motif or configuration is meant, an enzymatic nucleic acid molecule comprising a motif as is generally described in Eckstein et al., U.S. Pat. No. 6,127,173 and in Kore et al., 1998, Nucleic Acids Research 26, 4116-4120. G-cleavers possess endonuclease activity to cleave RNA substrates having a cleavage triplet NYN/, where N is a nucleotide, Y is uridine or cytidine and/represents the cleavage site. G-cleavers can be chemically modified.

By “zinzyme” motif or configuration is meant, an enzymatic nucleic acid molecule comprising a motif as is generally described in Beigelman et al., International PCT publication No. WO 99/55857 and U.S. patent application Ser. No. 09/918,728. Zinzymes possess endonuclease activity to cleave RNA substrates having a cleavage triplet including but not limited to, YG/Y, where Y is uridine or cytidine, and G is guanosine and/represents the cleavage site. Zinzymes can be chemically modified to increase nuclease stability through various substitutions, including substituting 2′-O-methyl guanosine nucleotides for guanosine nucleotides. In addition, differing nucleotide and/or non-nucleotide linkers can be used to substitute the 5′-gaaa-2′ loop of the motif. Zinzymes represent a non-limiting example of an enzymatic nucleic acid molecule that does not require a ribonucleotide (2′-OH) group within its own nucleic acid sequence for activity.

By “amberzyme” motif or configuration is meant, an enzymatic nucleic acid molecule comprising a motif as is generally described in Beigelman et al., International PCT publication No. WO 99/55857 and U.S. patent application Ser. No. 09/476,387. Amberzymes possess endonuclease activity to cleave RNA substrates having a cleavage triplet NG/N, where N is a nucleotide, G is guanosine, and/represents the cleavage site. Amberzymes can be chemically modified to increase nuclease stability. In addition, differing nucleoside and/or non-nucleoside linkers can be used to substitute the 5′-gaaa-3′ loops of the motif. Amberzymes represent a non-limiting example of an enzymatic nucleic acid molecule that does not require a ribonucleotide (2′-OH) group within its own nucleic acid sequence for activity.

By ‘DNAzyme’ is meant, an enzymatic nucleic acid molecule that does not require the presence of a 2′-OH group within its own nucleic acid sequence for activity. In particular embodiments, the enzymatic nucleic acid molecule can have an attached linker or linkers or other attached or associated groups, moieties, or chains containing one or more nucleotides with 2′-OH groups. DNAzymes can be synthesized chemically or expressed endogenously in vivo, by means of a single stranded DNA vector or equivalent thereof. Non-limiting examples of DNAzymes are generally reviewed in Usman et al., U.S. Pat. No., 6,159,714; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. The “10-23” DNAzyme motif is one particular type of DNAzyme that was evolved using in vitro selection as generally described in Joyce et al., U.S. Pat. No. 5,807,718 and Santoro et al., supra. Additional DNAzyme motifs can be selected for using techniques similar to those described in these references, and hence, are within the scope of the present invention.

By “nucleic acid sensor molecule” or “allozyme” as used herein is meant a nucleic acid molecule comprising an enzymatic domain and a sensor domain, where the enzymatic nucleic acid domain's ability to catalyze a chemical reaction is dependent on the interaction with a target signaling molecule, such as a nucleic acid, polynucleotide, oligonucleotide, peptide, polypeptide, or protein, for example HBV RT, HBV RT primer, or HBV Enhancer I sequence. The introduction of chemical modifications, additional functional groups, and/or linkers, to the nucleic acid sensor molecule can provide enhanced catalytic activity of the nucleic acid sensor molecule, increased binding affinity of the sensor domain to a target nucleic acid, and/or improved nuclease/chemical stability of the nucleic acid sensor molecule, and are hence within the scope of the present invention (see for example Usman et al., U.S. patent application Ser. No. 09/877,526, George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., U.S. patent application Ser. No. 09/205,520).

By “sensor component” or “sensor domain” of the nucleic acid sensor molecule as used herein is meant, a nucleic acid sequence (e.g., RNA or DNA or analogs thereof) which interacts with a target signaling molecule, for example a nucleic acid sequence in one or more regions of a target nucleic acid molecule or more than one target nucleic acid molecule, and which interaction causes the enzymatic nucleic acid component of the nucleic acid sensor molecule to either catalyze a reaction or stop catalyzing a reaction. In the presence of target signaling molecule of the invention, such as HBV RT, HBV RT primer, or HBV Enhancer I sequence, the ability of the sensor component, for example, to modulate the catalytic activity of the nucleic acid sensor molecule, is altered or diminished in a manner that can be detected or measured. The sensor component can comprise recognition properties relating to chemical or physical signals capable of modulating the nucleic acid sensor molecule via chemical or physical changes to the structure of the nucleic acid sensor molecule. The sensor component can be derived from a naturally occurring nucleic acid binding sequence, for example, RNAs that bind to other nucleic acid sequences in vivo. Alternately, the sensor component can be derived from a nucleic acid molecule (aptamer), which is evolved to bind to a nucleic acid sequence within a target nucleic acid molecule. The sensor component can be covalently linked to the nucleic acid sensor molecule, or can be non-covalently associated. A person skilled in the art will recognize that all that is required is that the sensor component is able to selectively modulate the activity of the nucleic acid sensor molecule to catalyze a reaction.

By “target molecule” or “target signaling molecule” is meant a molecule capable of interacting with a nucleic acid sensor molecule, specifically a sensor domain of a nucleic acid sensor molecule, in a manner that causes the nucleic acid sensor molecule to be active or inactive. The interaction of the signaling agent with a nucleic acid sensor molecule can result in modification of the enzymatic nucleic acid component of the nucleic acid sensor molecule via chemical, physical, topological, or conformational changes to the structure of the molecule, such that the activity of the enzymatic nucleic acid component of the nucleic acid sensor molecule is modulated, for example is activated or inactivated. Signaling agents can comprise target signaling molecules such as macromolecules, ligands, small molecules, metals and ions, nucleic acid molecules including but not limited to RNA and DNA or analogs thereof, proteins, peptides, antibodies, polysaccharides, lipids, sugars, microbial or cellular metabolites, pharmaceuticals, and organic and inorganic molecules in a purified or unpurified form, for example HBV RT or HBV RT primer.

By “sufficient length” is meant a nucleic acid molecule long enough to provide the intended function under the expected condition. For example, a nucleic acid molecule of the invention needs to be of “sufficient length” to provide stable binding to a target site under the expected binding conditions and environment. In another non-limiting example, for the binding arms of an enzymatic nucleic acid, “sufficient length” means that the binding arm sequence is long enough to provide stable binding to a target site under the expected reaction conditions and environment. The binding arms are not so long as to prevent useful turnover of the nucleic acid molecule. By “stably interact” is meant interaction of the oligonucleotides with target nucleic acid (e.g., by forming hydrogen bonds with complementary nucleotides in the target under physiological conditions) that is sufficient for the intended purpose (e.g., cleavage of target RNA by an enzyme).

By “equivalent” RNA to HBV or HCV is meant to include those naturally occurring RNA molecules having homology (partial or complete) to HBV or HCV proteins or encoding for proteins with similar function as HBV or HCV in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like.

The term “component” of HBV or HCV as used herein refers to a peptide or protein subunit expressed from a HBV or HCV gene.

By “homology” is meant the nucleotide sequence of two or more nucleic acid molecules is partially or completely identical.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two or more non-contiguous substrate sequences or two or more non-contiguous sequence portions of an antisense molecule can be complementary to a target sequence, or both. For a review of current antisense strategies, see Schmajuk et al., 1999, J. Biol. Chem., 274, 21783-21789, Delihas et al, 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. Antisense molecules of the instant invention can include 2-5A antisense chimera molecules. In addition, antisense DNA can be used to target RNA by means of DNA-RNA interactions, thereby activating RNase H, which digests the target RNA in the duplex. The antisense oligonucleotides can comprise one or more RNAse H activating region that is capable of activating RNAse H cleavage of a target RNA. Antisense DNA can be synthesized chemically or expressed via the use of a single stranded DNA expression vector or equivalent thereof.

By “RNase H activating region” is meant a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothioate (for example, at least four of the nucleotides are phosphorothiote substitutions; more specifically, 4-11 of the nucleotides are phosphorothiote substitutions), phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

By “2-5A antisense” or “2-5A antisense chimera” is meant an antisense oligonucleotide containing a 5′-phosphorylated 2′-5′-linked adenylate residue. These chimeras bind to target RNA in a sequence-specific manner and activate a cellular 2-5A-dependent ribonuclease which, in turn, cleaves the target RNA (Torrence et al., 1993 Proc. Natl. Acad. Sci. USA 90, 1300; Silverman et al., 2000, Methods Enzymol., 313, 522-533; Player and Torrence, 1998, Pharmacol. Ther., 78, 55-113).

By “triplex nucleic acid” or “triplex oligonucleotide” it is meant a polynucleotide or oligonucleotide that can bind to a double-stranded DNA in a sequence-specific manner to form a triple-strand helix. Formation of such triple helix structure has been shown to modulate transcription of the targeted gene (Duval-Valentin et al., 1992, Proc. Natl. Acad. Sci. USA, 89, 504). Triplex nucleic acid molecules of the invention also include steric blocker nucleic acid molecules that bind to the Enhancer I region of HBV DNA (plus strand and/or minus strand) and prevent translation of HBV genomic DNA.

The term “single stranded RNA” (ssRNA) as used herein refers to a naturally occurring or synthetic ribonucleic acid molecule comprising a linear single strand, for example a ssRNA can be a messenger RNA (mRNA), transfer RNA (tRNA), ribosomal RNA (rRNA) etc. of a gene.

The term “single stranded DNA” (ssDNA) as used herein refers to a naturally occurring or synthetic deoxyribonucleic acid molecule comprising a linear single strand, for example, a ssDNA can be a sense or antisense gene sequence or EST (Expressed Sequence Tag).

The term “allozyme” as used herein refers to an allosteric enzymatic nucleic acid molecule, see for example George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842.

The term “2-5A chimera” as used herein refers to an oligonucleotide containing a 5′-phosphorylated 2′-5′-linked adenylate residue. These chimeras bind to target RNA in a sequence-specific manner and activate a cellular 2-5A-dependent ribonuclease which, in turn, cleaves the target RNA (Torrence et al., 1993 Proc. Natl. Acad. Sci. USA 90, 1300; Silverman et al., 2000, Methods Enzymol., 313, 522-533; Player and Torrence, 1998, Pharmacol. Ther., 78, 55-113).

The term “double stranded RNA” or “dsRNA” as used herein refers to a double stranded RNA molecule capable of RNA interference “RNAi”, including short interfering RNA “siRNA” see for example Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914.

By “gene” it is meant, a nucleic acid that encodes an RNA, for example, nucleic acid sequences including, but not limited to, structural genes encoding a polypeptide.

By “complementarity” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., ribozyme cleavage, antisense or triple helix modulation. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

As used herein “cell” is used in its usual biological sense, and does not refer to an entire multicellular organism, e.g., specifically does not refer to a human. The cell can be present in an organism, e.g., birds, plants and mammals such as humans, cows, sheep, apes, monkeys, swine, dogs, and cats. The cell can be prokaryotic (e.g., bacterial cell) or eukaryotic (e.g., mammalian or plant cell).

By “HBV proteins” or “HCV proteins” is meant, a protein or a mutant protein derivative thereof, comprising sequence expressed and/or encoded by the HBV genome.

By “highly conserved sequence region” is meant a nucleotide sequence of one or more regions in a target gene does not vary significantly from one generation to the other or from one biological system to the other.

By “highly conserved nucleic acid binding region” is meant an amino acid sequence of one or more regions in a target protein that does not vary significantly from one generation to the other or from one biological system to the other.

By “related to the levels of HBV” is meant that the reduction of HBV expression (specifically HBV gene) RNA levels and thus reduction in the level of the respective protein will relieve, to some extent, the symptoms of the disease or condition.

By “related to the levels of HCV” is meant that the reduction of HCV expression (specifically HCV gene) RNA levels and thus reduction in the level of the respective protein will relieve, to some extent, the symptoms of the disease or condition.

By “RNA” is meant a molecule comprising at least one ribonucleotide residue. By “ribonucleotide” is meant a nucleotide with a hydroxyl group at the 2′ position of a β-D-ribo-furanose moiety.

By “vector” is meant any nucleic acid- and/or viral-based technique used to express and/or deliver a desired nucleic acid.

By “patient” is meant an organism, which is a donor or recipient of explanted cells or the cells themselves. “Patient” also refers to an organism to which the nucleic acid molecules of the invention can be administered. In one embodiment, a patient is a mammal or mammalian cells. In another embodiment, a patient is a human or human cells.

Other features and advantages of the invention will be apparent from the following description of the preferred embodiments thereof, and from the claims.

### DESCRIPTION OF THE PREFERRED EMBODIMENTS

First the drawings will be described briefly.

### DRAWINGS

FIG. 1 shows the secondary structure model for seven different classes of enzymatic nucleic acid molecules. Arrow indicates the site of cleavage. --------- indicate the target sequence. Lines interspersed with dots are meant to indicate tertiary interactions. - is meant to indicate base-paired interaction. Group I Intron: P1-P9.0 represent various stem-loop structures (Cech et al., 1994, Nature Struc. Bio., 1, 273). RNase P (M1RNA): EGS represents external guide sequence (Forster et al., 1990, Science, 249, 783; Pace et al., 1990, J. Biol. Chem., 265, 3587). Group II Intron: 5′SS means 5′ splice site; 3′SS means 3′-splice site; IBS means intron binding site; EBS means exon binding site (Pyle et al., 1994, Biochemistry, 33, 2716). VS RNA: I-VI are meant to indicate six stem-loop structures; shaded regions are meant to indicate tertiary interaction (Collins, International PCT Publication No. WO 96/19577). HDV Ribozyme: I-IV are meant to indicate four stem-loop structures (Been et al., U.S. Pat. No. 5,625,047). Hammerhead Ribozyme: I-III are meant to indicate three stem-loop structures; stems I-III can be of any length and may be symmetrical or asymmetrical (Usman et al., 1996, Curr. Op. Struct. Bio., 1, 527). Hairpin Ribozyme: Helix 1, 4 and 5 can be of any length; Helix 2 is between 3 and 8 base-pairs long; Y is a pyrimidine; Helix 2 (H2) is provided with a least 4 base pairs (i.e., n is 1, 2, 3 or 4) and helix 5 can be optionally provided of length 2 or more bases (preferably 3-20 bases, i.e., m is from 1-20 or more). Helix 2 and helix 5 may be covalently linked by one or more bases (i.e., r is ≧1 base). Helix 1, 4 or 5 may also be extended by 2 or more base pairs (e.g., 4-20 base pairs) to stabilize the ribozyme structure, and preferably is a protein binding site. In each instance, each N and N′ independently is any normal or modified base and each dash represents a potential base-pairing interaction. These nucleotides may be modified at the sugar, base or phosphate. Complete base-pairing is not required in the helices, but is preferred. Helix 1 and 4 can be of any size (i.e., o and p is each independently from 0 to any number, e.g., 20) as long as some base-pairing is maintained. Essential bases are shown as specific bases in the structure, but those in the art will recognize that one or more may be modified chemically (abasic, base, sugar and/or phosphate modifications) or replaced with another base without significant effect. Helix 4 can be formed from two separate molecules, i.e., without a connecting loop. The connecting loop when present may be a ribonucleotide with or without modifications to its base, sugar or phosphate. “q”≧is 2 bases. The connecting loop can also be replaced with a non-nucleotide linker molecule. H refers to bases A, U, or C. Y refers to pyrimidine bases. “______” refers to a covalent bond. (Burke et al., 1996, Nucleic Acids & Mol. Biol., 10, 129; Chowrira et al., U.S. Pat. No. 5,631,359).

FIG. 2 shows examples of chemically stabilized ribozyme motifs. HH Rz, represents hammerhead ribozyme motif (Usman et al., 1996, Curr. Op. Struct. Bio., 1, 527); NCH Rz represents the NCH ribozyme motif (Ludwig & Sproat, International PCT Publication No. WO 98/58058); G-Cleaver, represents G-cleaver ribozyme motif (Kore et al., 1998, Nucleic Acids Research, 26, 4116-4120). N or n, represent independently a nucleotide which may be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

FIG. 3 shows an example of the Amberzyme ribozyme motif that is chemically stabilized (see, for example, Beigelman et al., International PCT publication No. WO 99/55857; also referred to as Class I Motif). The Amberzyme motif is a class of enzymatic nucleic acid molecules that do not require the presence of a ribonucleotide (2′-OH) group for activity.

FIG. 4 shows an example of the Zinzyme A ribozyme motif that is chemically stabilized (see, for example, International PCT publication No. WO 99/55857; also referred to as Class A Motif). The Zinzyme motif is a class of enzymatic nucleic acid molecules that do not require the presence of a ribonucleotide (2′-OH) group for activity.

FIG. 5 shows an example of a DNAzyme motif described by Santoro et al., 1997, PNAS, 94, 4262.

FIG. 6 is a bar graph showing the percent change in serum HBV DNA levels following fourteen days of ribozyme treatment in HBV transgenic mice. Ribozymes targeting sites 273 (RPI.18341) and 1833 (RPI.18371) of HBV RNA administerd via continuous s.c. infusion at 10, 30, and 100 mg/kg/day are compared to continuous s.c. infusion administration of scrambled attenuated core ribozyme and saline controls, and orally administered 3TC® (300 mg/kg/day) and saline controls.

FIG. 7 is a bar graph showing the mean serum HBV DNA levels following fourteen days of ribozyme treatment in HBV transgenic mice. Ribozymes targeting sites 273 (RPI.18341) and 1833 (RPI.18371) of HBV RNA administerd via continuous s.c. infusion at 10, 30, and 100 mg/kg/day are compared to continuous s.c. infusion administration of scrambled attenuated core ribozyme and saline controls, and orally administered 3TC® (300 mg/kg/day) and saline controls.

FIG. 8 is a bar graph showing the decrease in serum HBV DNA (log) levels following fourteen days of ribozyme treatment in HBV transgenic mice. Ribozymes targeting sites 273 (RPI.18341) and 1833 (RPI.18371) of HBV RNA administerd via continuous s.c. infusion at 10, 30, and 100 mg/kg/day are compared to continuous s.c. infusion administration of scrambled attenuated core ribozyme and saline controls, and orally administered 3TC® (300 mg/kg/day) and saline controls.

FIG. 9 is a bar graph showing the decrease in HBV DNA in HepG2.2.15 cells after treatment with ribozymes targeting sites 273 (RPI.18341), 1833 (RPI.18371), 1874 (RPI.18372), and 1873 (RPI.18418) of HBV RNA as compared to a scrambled attenuated core ribozyme (RPI.20995).

FIG. 10 is a bar graph showing reduction in HBsAg levels following treatment of HepG2 cells with anti-HBV arm, stem, and loop-variant ribozymes (RPI.18341, RPI.22644, RPI.22645, RPI.22646, RPI.22647, RPI.22648, RPI.22649, and RPI.22650) targeting site 273 of the HBV pregenomic RNA as compared to a scrambled attenuated core ribozyme (RPI.20599).

FIG. 11 is a bar graph showing reduction in HBsAg levels following treatment of HepG2 cells with RPI 18341 alone or in combination with Infergen®. At either 500 or 1000 units of Infergen®, the addition of 200 nM of RPI.18341 results in a 75-77% increase in anti-HBV activity as judged by the level of HBsAg secreted from the treated Hep G2 cells. Conversely, the anti-HBV activity of RPI.18341 (at 200 nM) is increased 31-39% when used in combination of 500 or 1000 units of Infergen®.

FIG. 12 is a bar graph showing reduction in HBsAg levels following treatment of HepG2 cells with RPI 18341 alone or in combination with Lamivudine. At 25 nM Lamivudine (3TC®), the addition of 100 nM of RPI.18341 results in a 48% increase in anti-HBV activity as judged by the level of HBsAg secreted from treated Hep G2 cells. Conversely, the anti-HBV activity of RPI. 18341 (at 100 nM) is increased 31% when used in combination with 25 mM Lamivudine.

FIG. 13 shows a scheme which outlines the steps involved in HBV reverse transcription. The HBV polymerase/reverse transcriptase binds to the 5′-stem-loop of the HBV pregenomic RNA and synthesizes a primer from the UUCA template. The reverse transcriptase and tetramer primer are translocated to the 3′-DR1 site. The RT primer binds to the UUCA sequence in the DR1 element and minus strand synthesis begins.

FIG. 14 shows a non-limiting example of inhibition of HBV reverse transcription. A decoy molecule binds to the HBV RT primer, thereby preventing translocation of the RT to the 3′-DR1 site and preventing minus strand synthesis.

FIG. 15 shows data of a HBV nucleic acid screen of 2′-O-allyl modified nucleic acid molecules. The levels of HbsAg were determined by ELISA. Inhibition of HBV is correlated to HBsAg antigen levels.

FIG. 16 shows data of a HBV nucleic acid screen of 2′-O-methyl modified nucleic acid molecules. The levels of HbsAg were determined by ELISA. Inhibition of HBV is correlated to HBsAg antigen levels.

FIG. 17 shows dose response data of 2′-O-methyl modified nucleic acid molecules targeting the HBV reverse transcriptase primer compared to levels of HBsAg.

FIG. 18 shows data of nucleic acid screen of nucleic acid molecules (200 nM) targeting the HBV Enhancer I core region compared to levels of HBsAg.

FIG. 19 shows data of nucleic acid screen of nucleic acid molecules (400 nM) targeting the HBV Enhancer I core region compared to levels of HBsAg.

FIG. 20 shows dose response data of nucleic acid molecules targeting the HBV Enhancer I core region compared to levels of HBsAg.

FIG. 21 shows a graph depicting HepG2.2.15 tumor growth in athymic nu/nu female mice as tumor volume (mm³) vs time (days).

FIG. 22 shows a graph depicting HepG2.2.15 tumor growth in athymic nu/nu female mice as tumor volume (mm³) vs time (days). Inoculated HepG2.2.15 cells were selected for antibiotic resistance to G418 before introduction into the mouse.

FIG. 23 is a schematic representation of the Dual Reporter System utilized to demonstrate enzymatic nucleic acid mediated reduction of luciferase activity in cell culture.

FIG. 24 shows a schematic view of the secondary structure of the HCV 5′UTR (Brown et al., 1992, Nucleic Acids Res., 20, 5041-45; Honda et al., 1999, J. Virol., 73, 1165-74). Major structural domains are indicated in bold. Enzymatic nucleic acid cleavage sites are indicated by arrows. Solid arrows denote sites amenable to amino-modified enzymatic nucleic acid inhibition. Lead cleavage sites (195 and 330) are indicated with oversized solid arrows.

FIG. 25 shows a non-limiting example of a nuclease resistant enzymatic nucleic acid molecule. Binding arms are indicated as stem I and stem III. Nucleotide modifications are indicated as follows: 2′-O-methyl nucleotides, lowercase; ribonucleotides, uppercase G, A; 2′-amino-uridine, u; inverted 3′-3′ deoxyabasic, B. The positions of phosphorothioate linkages at the 5′-end of each enzymatic nucleic acid are indicated by subscript “s”. H indicates A, C or U ribonucleotide, N′ indicates A, C G or U ribonucleotide in substrate, n indicates base complementary to the N′. The U4 and U7 positions in the catalytic core are indicated.

FIG. 26 is a set of bar graphs showing enzymatic nucleic acid mediated inhibition of HCV-luciferase expression in OST7 cells. OST7 cells were transfected with complexes containing reporter plasmids (2 μg/mL), enzymatic nucleic acids (100 nM) and lipid. The ratio of HCV-firefly luciferase luminescence/Renilla luciferase luminescence was determined for each enzymatic nucleic acid tested and was compared to treatment with the ICR, an irrelevant control enzymatic nucleic acid lacking specificity to the HCV 5′UTR (adjusted to 1). Results are reported as the mean of triplicate samples ±SD. In FIG. 26A, OST7 cells were treated with enzymatic nucleic acids (100 nM) targeting conserved sites (indicated by cleavage site) within the HCV 5′UTR. In FIG. 26B, OST7 cells were treated with a subset of enzymatic nucleic acids to lead HCV sites (indicated by cleavage site) and corresponding attenuated core (AC) controls. Percent decrease in firefly/Renilla luciferase ratio after treatment with active enzymatic nucleic acids as compared to treatment with corresponding ACs is shown when the decrease is ≧50% and statistically significant. Similar results were obtained with 50 nM enzymatic nucleic acid.

FIG. 27 is a series of line graphs showing the dose-dependent inhibition of HCV/luciferase expression following enzymatic nucleic acid treatment. Active enzymatic nucleic acid was mixed with corresponding AC to maintain a 100 nM total oligonucleotide concentration and the same lipid charge ratio. The concentration of active enzymatic nucleic acid for each point is shown. FIGS. 27A-E shows enzymatic nucleic acids targeting sites 79, 81, 142, 195, or 330, respectively. Results are reported as the mean of triplicate samples ±SD.

FIG. 28 is a set of bar graphs showing reduction of HCV/luciferase RNA and inhibition of HCV-luciferase expression in OST7 cells. OST7 cells were transfected with complexes containing reporter plasmids (2 μg/ml), enzymatic nucleic acids, BACs or SACs (50 mM) and lipid. Results are reported as the mean of triplicate samples ±SD. In FIG. 28A the ratio of HCV-firefly luciferase RNA/Renilla luciferase RNA is shown for each enzymatic nucleic acid or control tested. As compared to paired BAC controls (adjusted to 1), luciferase RNA levels were reduced by 40% and 25% for the site 195 or 330 enzymatic nucleic acids, respectively. In FIG. 28B the ratio of HCV-firefly luciferase luminescence/Renilla luciferase luminescence is shown after treatment with site 195 or 330 enzymatic nucleic acids or paired controls. As compared to paired BAC controls (adjusted to 1), inhibition of protein expression was 70% and 40% for the site 195 or 330 enzymatic nucleic acids, respectively P<0.01.

FIG. 29 is a set a bar graphs showing interferon (IFN) alpha 2a and 2b dose response in combination with site 195 anti-HCV enzymatic nucleic acid treatment. FIG. 29A shows data for IFN alfa 2a treatment. FIG. 29B shows data for IFN alfa 2b treatment. Viral yield is reported from HeLa cells pretreated with IFN in units/ml (U/ml) as indicated for 4 h prior to infection and then treated with either 200 nM control (SAC) or site 195 anti-HCV enzymatic nucleic acid (195 RZ) for 24 h after infection. Cells were infected with a MOI=0.1 for 30 min and collected at 24 h post infection. Error bars represent the S.D. of the mean of triplicate determinations.

FIG. 30 is a line graph showing site 195 anti-HCV enzymatic nucleic acid dose response in combination with interferon (IFN) alpha 2a and 2b pretreatment. Viral yield is reported from HeLa cells pretreated for 4 h with or without IFN and treated with doses of site 195 anti-HCV enzymatic nucleic acid (195 RZ) as indicated for 24 h after infection. Anti-HCV enzymatic nucleic acid was mixed with control oligonucleotide (SAC) to maintain a constant 200 nM total dose of nucleic acid for delivery. Cells were infected with a MOI=0.1 for 30 min and collected at 24 h post infection. Error bars represent the S.D. of the mean of triplicate determinations.

FIG. 31 is a set of bar graphs showing data from consensus interferon (CIFN)/enzymatic nucleic acid combination treatment. FIG. 31A shows CIFN dose response with site 195 anti-HCV enzymatic nucleic acid treatment. Viral yield is reported from cells pretreated with CIFN in units/ml (U/ml) as indicated and treated with either 200 nM control (SAC) or site 195 anti-HCV enzymatic nucleic acid (195 RZ). FIG. 31B shows site 195 anti-HCV enzymatic nucleic acid dose response with CIFN pretreatment. Viral yield is reported from cells pretreated with or without CIFN and treated with concentrations of site 195 anti-HCV enzymatic nucleic acid (195 RZ) as indicated. Anti-HCV enzymatic nucleic acid was mixed with control oligonucleotide (SAC) to maintain a constant 200 nM total dose of nucleic acid for delivery. Cells were infected with a MOI=0.1 for 30 min. and collected at 24 h post infection. Error bars represent the S.D. of the mean of triplicate determinations.

FIG. 32 is a bar graph showing enzymatic nucleic acid activity and enhanced antiviral effect of an anti-HCV enzymatic nucleic acid targeting site 195 used in combination with consensus interferon (CIFN). Viral yield is reported from cells treated as indicated. BAC, cells were treated with 200 nM BAC (binding attenuated control) for 24 h after infection; CIFN+BAC, cells were treated with 12.5 U/ml CIFN for 4 h prior to infection and with 200 nM BAC for 24 h after infection; 195 RZ, cells were treated with 200 nM site 195 anti-HCV enzymatic nucleic acid for 24 h after infection; CIFN+195 RZ, cells were treated with 12.5 U/ml CIFN for 4 h prior to infection and with 200 nM site 195 anti-HCV enzymatic nucleic acid for 24 h after infection. Cells were infected with a MOI=0.1 for 30 min. Error bars represent the S.D. of the mean of triplicate determinations.

FIG. 33 is a bar graph showing inhibition of a HCV-PV chimera replication by treatment with zinzyme enzymatic nucleic acid molecules targeting different sites within the HCV 5′-UTR compared to a scrambled attenuated core control (SAC) zinzyme.

FIG. 34 is a bar graph showing inhibition of a HCV-PV chimera replication by antisense nucleic acid molecules targeting conserved regions of the HCV 5′-UTR compared to scrambled antisense controls.

FIG. 35 shows the structure of compounds (2-5A) utilized in the study. “X” denotes the position of oxygen (O) in analog I or sulfur (S) in thiophosphate (P═S) analog II. The 2-5A compounds were synthesized, deprotected and purified as described herein utilizing CPG support with 3′-inverted abasic nucleotide. For chain extension 5′-O-(4,4′-dimetoxytrityl)-3′-O-(tert-butyldimethylsilyl)-N-6-benzoyladenosine-2-cyanoethyl-N,N-diisopropyl-phosphoramidite (Chem. Genes Corp., Waltham, Mass.) was employed. Introduction of a 5′-terminal phosphate (analog I) or thiophosphate (analog II) group was performed with “Chemical Phosphorylation Reagent” (Glen Research, Sterling, Va.). Structures of the final compounds were confirmed by MALDI-TOF analysis.

FIG. 36 is a bar graph showing ribozyme activity and enhanced antiviral effect. (A) Interferon/ribozyme combination treatment. (B) 2-5A/ribozyme combination treatment. HeLa cells seeded in 96-well plates (10,000 cells per well) were pretreated as indicated for 4 hours. For pretreatment, SAC (RPI 17894), RZ (RPI 13919), and 2-5A analog I (RPI 21096) (200 nM) were complexed with lipid cytofectin. Cells were then infected with HCV-PV at a multiplicity of infection of 0.1. Virus inoculum was replaced after 30 minutes with media containing 5% serum and 100 nM RZ or SAC as indicated, complexed with cytofectin RPI.9778. After 20 hours, cells were lysed by 3 freeze/thaw cycles and virus was quantified by plaque assay. Plaque forming units (PFU)/ml are shown as the mean of triplicate samples +SEM. The absolute amount of viral yield in treated cells varied from day to day, presumably due to day to day variations in cell plating and transfection complexation. None, normal media; IFN, 10 U/ml consensus interferon; SAC, scrambled arm attenuated core control (RPI 17894); RZ, anti-HCV ribozyme (RPI 13919); 2-5A, (RPI 21096).

FIG. 37 is a graph showing the inhibition of viral replication with anti-HCV ribozyme (RPI 13919) or 2-5A (RPI 21096) treatment. HeLa cells were treated as described in FIG. 36 except that there was no pretreatment and 200 nM oligonucleotide was used for treatment. 2-5A P═S contains a 5′-terminal thiophosphate (RPI21095) (see FIG. 35).

FIG. 38 is a bar graph showing anti-HCV ribozyme in combination with 2-5A treatment. HeLa cells were treated as described in FIG. 37 except concentrations were co-varied as shown to maintain a constant 200 nM total oligonucleotide dose for transfection. Cells treated with 50 nM anti-HCV ribozyme (RPI 13919) (middle bars) were also treated with 150 nM SAC (RPI 17894) or 2-5A (RPI 21096); likewise, cells treated with 100 nM anti-HCV ribozyme (bars at right) were also treated with 100 nM SAC or 2-5A.

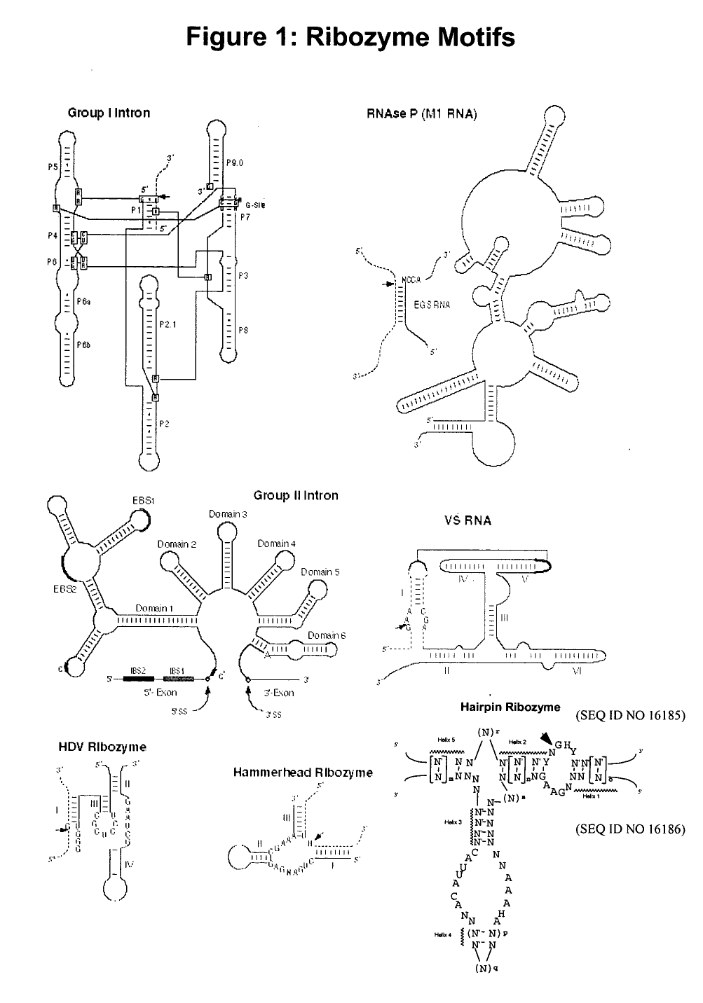

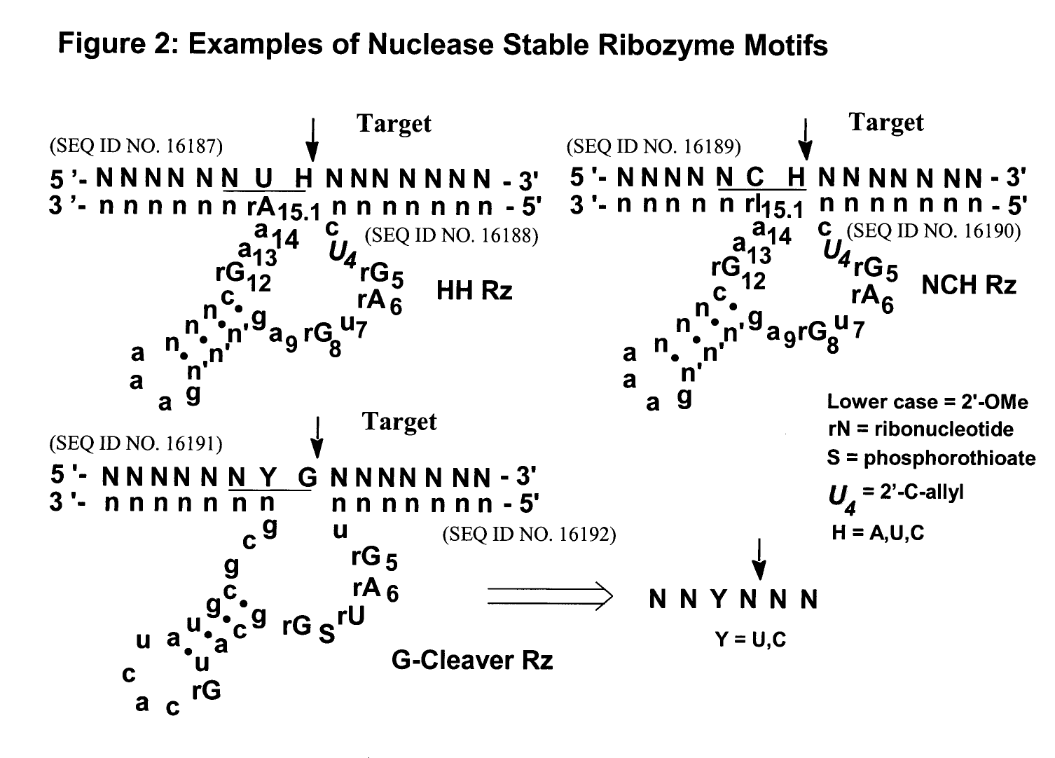

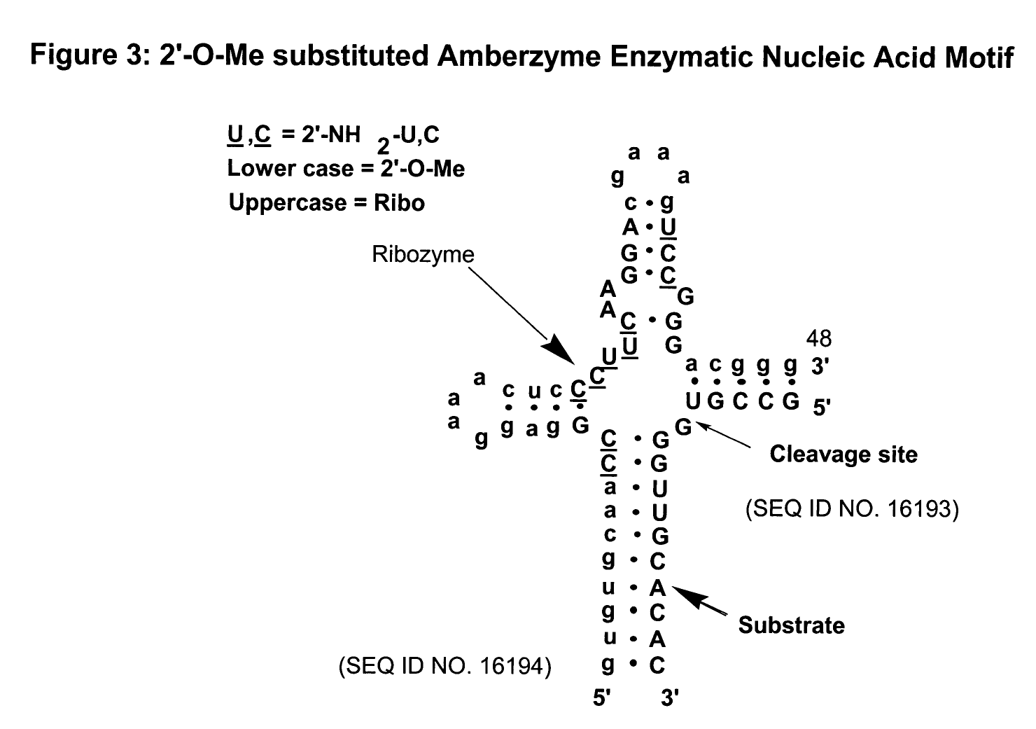

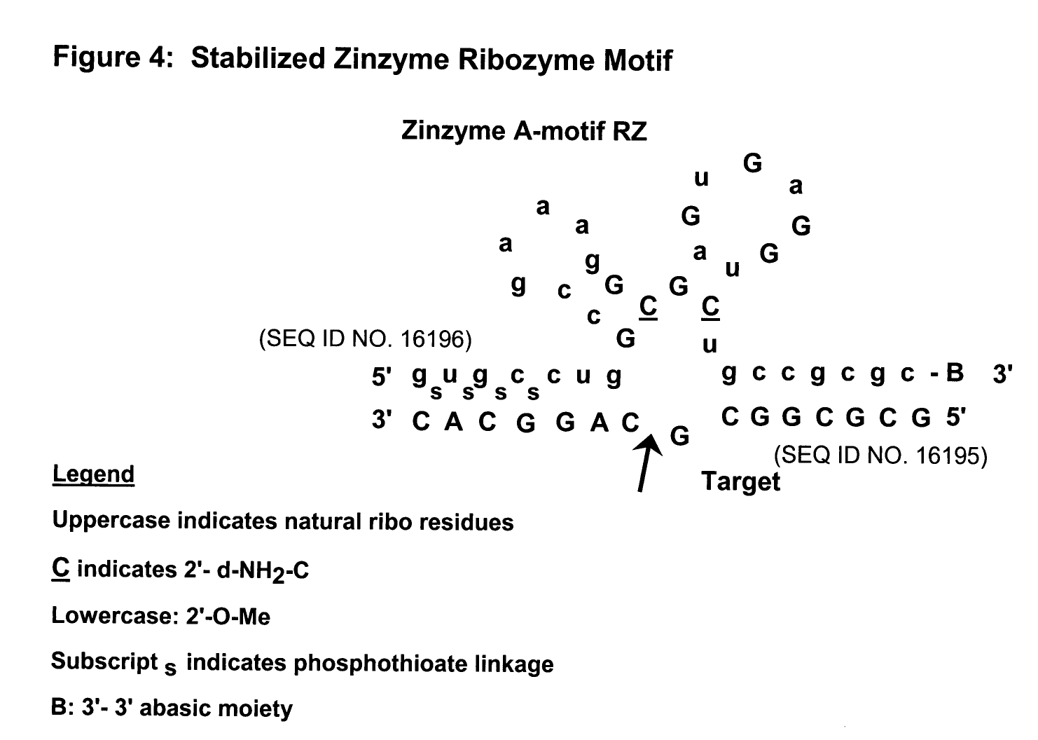

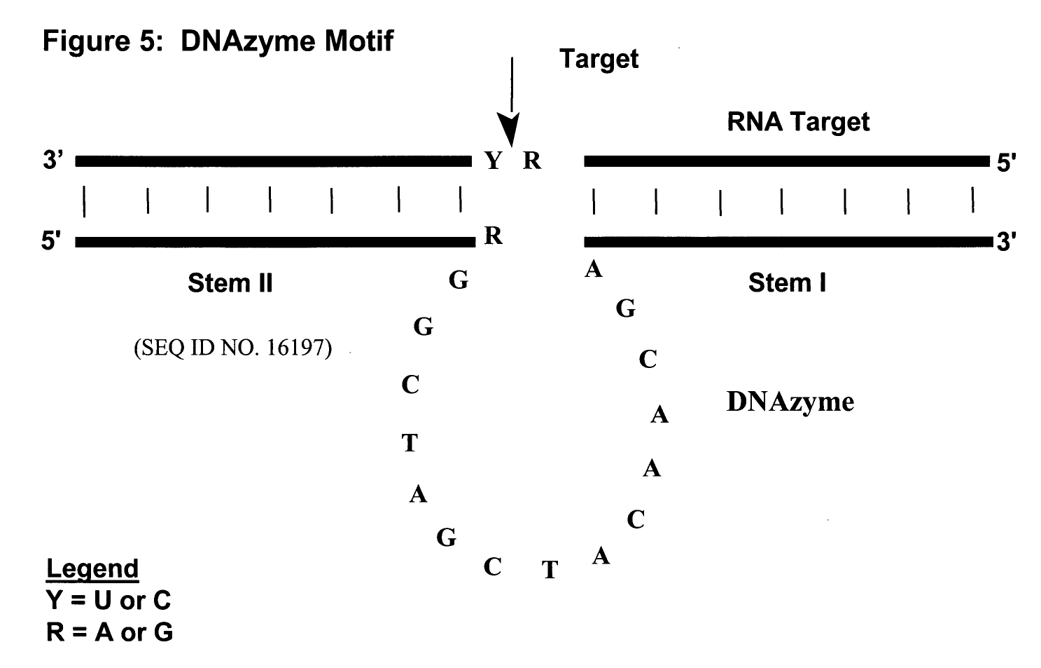

### MECHANISM OF ACTION OF NUCLEIC ACID MOLECULES OF THE INVENTION

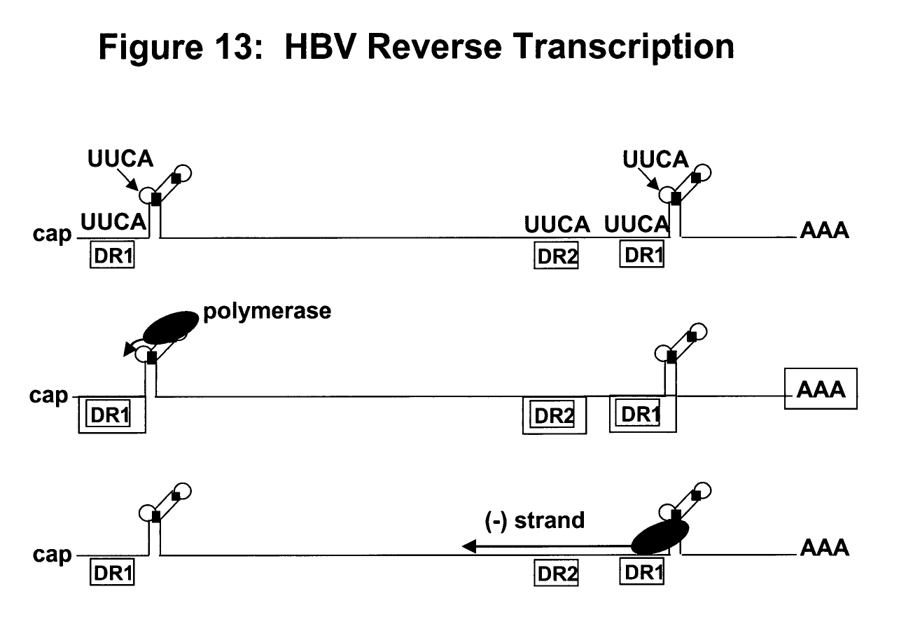

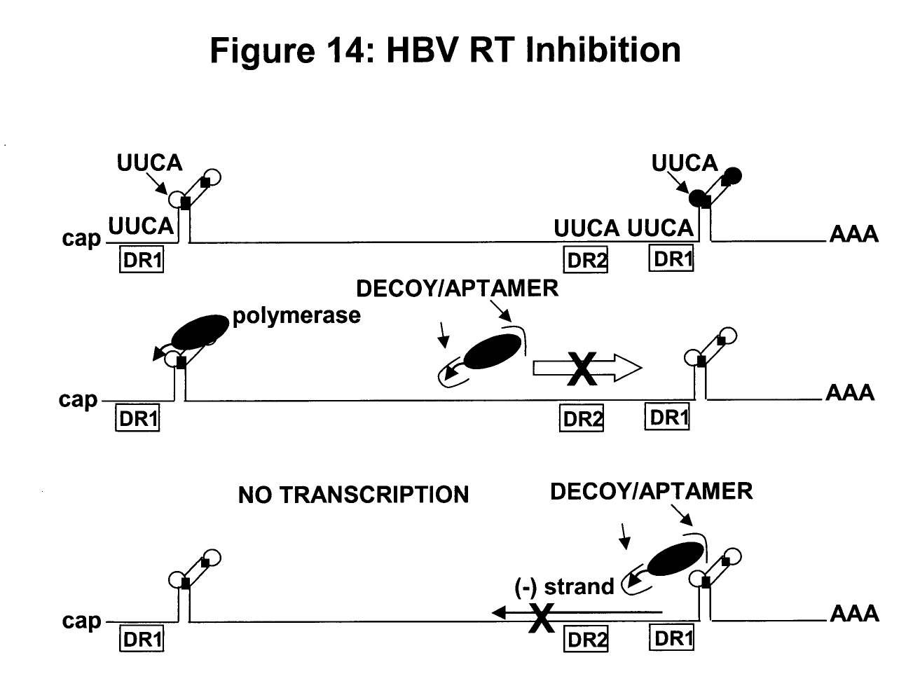

Decoy: Nucleic acid decoy molecules are mimetics of naturally occurring nucleic acid molecules or portions of naturally occurring nucleic acid molecules that can be used to modulate the function of a specific protein or a nucleic acid whose activity is dependant on interaction with the naturally occurring nucleic acid molecule. Decoys modulate the function of a target protein or nucleic acid by competing with authentic nucleic acid binding to the ligand of interest. Often, the nucleic acid decoy is a truncated version of a nucleic acid sequence that is recognized, for example by a particular protein, such as a transcription factor or polymerase. Decoys can be chemically modified to increase binding affinity to the target ligand as well as to increase the enzymatic and chemical stability of the decoy. In addition, bridging and non-bridging linkers can be introduced into the decoy sequence to provide additional binding affinity to the target ligand. Decoy molecules of the invention that bind to an HCV or HBV target, such as HBV reverse transcriptase or HBV reverse transcriptase primer, or an enhancer region of the HBV pregenomic RNA, for example the Enhancer I element, modulate the transcription of RNA to DNA and therefore modulate expression of the pregenomic RNA of the virus (see FIGS. 13 and 14).

Aptamer: Nucleic acid aptamers can be selected to specifically bind to a particular ligand of interest (see for example Gold et al., U.S. Pat. No. 5,567,588 and U.S. Pat. No. 5,475,096, Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628). For example, the use of in vitro selection can be applied to evolve nucleic acid aptamers with binding specificity for HBV RT and/or HBV RT primer. Nucleic acid aptamers can include chemical modifications and linkers as described herein. Aptamer molecules of the invention that bind to a reverse transcriptase or reverse transcriptase primer, such as HBV reverse transcriptase or HBV reverse transcriptase primer, modulate the transcription of RNA to DNA and therefore modulate expression of the pregenomic RNA of the virus.

Antisense: Antisense molecules can be modified or unmodified RNA, DNA, or mixed polymer oligonucleotides and primarily function by specifically binding to matching sequences resulting in modulation of peptide synthesis (Wu-Pong, November 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA may result in nuclease degradation of the heteroduplex (Wu-Pong, supra; Crooke, supra). To date, the only backbone modified DNA chemistry which will act as substrates for RNase H are phosphorothioates, phosphorodithioates, and borontrifluoridates. Recently, it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., U.S. S No. 60/082,404 which was filed on Apr. 20, 1998; Hartmann et al., U.S. S No. 60/101,174 which was filed on Sep. 21, 1998) all of these are incorporated by reference herein in their entirety.

Antisense DNA can be used to target RNA by means of DNA-RNA interactions, thereby activating RNase H, which digests the target RNA in the duplex. Antisense DNA can be chemically synthesized or can be expressed via the use of a single stranded DNA intracellular expression vector or the equivalent thereof.

Triplex Forming Oligonucleotides (TFO): Single stranded oligonucleotide can be designed to bind to genomic DNA in a sequence specific manner. TFOs can be comprised of pyrimidine-rich oligonucleotides which bind DNA helices through Hoogsteen Base-pairing (Wu-Pong, supra). In addition, TFOs can be chemically modified to increase binding affinity to target DNA sequences. The resulting triple helix composed of the DNA sense, DNA antisense, and TFO disrupts RNA synthesis by RNA polymerase. The TFO mechanism can result in gene expression or cell death since binding may be irreversible (Mukhopadhyay & Roth, supra) 2′-5′ Oligoadenylates: The 2-5A system is an interferon-mediated mechanism for RNA degradation found in higher vertebrates (Mitra et al., 1996, Proc Nat Acad Sci USA 93, 6780-6785). Two types of enzymes, 2-5A synthetase and RNase L, are required for RNA cleavage. The 2-5A synthetases require double stranded RNA to form 2′-5′ oligoadenylates (2-5A). 2-5A then acts as an allosteric effector for utilizing RNase L, which has the ability to cleave single stranded RNA. The ability to form 2-5A structures with double stranded RNA makes this system particularly useful for modulation of viral replication.

(2′-5′) oligoadenylate structures can be covalently linked to antisense molecules to form chimeric oligonucleotides capable of RNA cleavage (Torrence, supra). These molecules putatively bind and activate a 2-5A-dependent RNase, the oligonucleotide/enzyme complex then binds to a target RNA molecule which can then be cleaved by the RNase enzyme. The covalent attachment of 2′-5′ oligoadenylate structures is not limited to antisense applications, and can be further elaborated to include attachment to nucleic acid molecules of the instant invention.

RNA interference (RNAi): RNA interference refers to the process of sequence specific post transcriptional gene silencing in animals mediated by short interfering RNAs (siRNA) (Fire et al., 1998, Nature, 391, 806). The corresponding process in plants is commonly referred to as post transcriptional gene silencing or RNA silencing and is also referred to as quelling in fungi. The process of post transcriptional gene silencing is thought to be an evolutionarily conserved cellular defense mechanism used to prevent the expression of foreign genes which is commonly shared by diverse flora and phyla (Fire et al., 1999, Trends Genet., 15, 358). Such protection from foreign gene expression may have evolved in response to the production of double stranded RNAs (dsRNA) derived from viral infection or the random integration of transposon elements into a host genome via a cellular response that specifically destroys homologous single stranded RNA or viral genomic RNA. The presence of dsRNA in cells triggers the RNAi response though a mechanism that has yet to be fully characterized. This mechanism appears to be different from the interferon response that results from dsRNA mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNA) (Berstein et al., 2001, Nature, 409, 363). Short interfering RNAs derived from dicer activity are typically about 21-23 nucleotides in length and comprise about 19 base pair duplexes. Dicer has also been implicated in the excision of 21 and 22 nucleotide small temporal RNAs (stRNA) from precursor RNA of conserved structure that are implicated in translational control (Hutvagner et al., 2001, Science, 293, 834). The RNAi response also features an endonuclease complex containing a siRNA, commonly referred to as an RNA-induced silencing complex (RISC), which mediates cleavage of single stranded RNA having sequence homologous to the siRNA. Cleavage of the target RNA takes place in the middle of the region complementary to the guide sequence of the siRNA duplex (Elbashir et al., 2001, Genes Dev., 15, 188).

Short interfering RNA mediated RNAi has been studied in a variety of systems. Fire et al., 1998, Nature, 391, 806, were the first to observe RNAi in C. Elegans. Wianny and Goetz, 1999, Nature Cell Biol., 2, 70, describes RNAi mediated by dsRNA in mouse embryos. Hammond et al., 2000, Nature, 404, 293, describe RNAi in Drosophila cells transfected with dsRNA. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates has revealed certain requirements for siRNA length, structure, chemical composition, and sequence that are essential to mediate efficient RNAi activity. These studies have shown that 21 nucleotide siRNA duplexes are most active when containing two nucleotide 3′-overhangs. Furthermore, substitution of one or both siRNA strands with 2′-deoxy or 2′-O-methyl nucleotides abolishes RNAi activity, whereas substitution of 3′-terminal siRNA nucleotides with deoxy nucleotides was shown to be tolerated. Mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end (Elbashir et al., 2001, EMBO J., 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309), however siRNA molecules lacking a 5′-phosphate are active when introduced exogenously, suggesting that 5′-phosphorylation of siRNA constructs may occur in vivo.

Enzymatic Nucleic Acid: Several varieties of naturally occurring enzymatic RNAs are presently known (Doherty and Doudna, 2001, Annu. Rev. Biophys. Biomol. Struct., 30, 457-475; Symons, 1994, Curr. Opin. Struct. Biol., 4, 322-30). In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495). Each can catalyze a series of reactions including the hydrolysis of phosphodiester bonds in trans (and thus can cleave other RNA molecules) under physiological conditions.

Nucleic acid molecules of this invention can block HBV or HCV protein expression and can be used to treat disease or diagnose disease associated with the levels of HBV or HCV.

The enzymatic nature of an enzymatic nucleic acid has significant advantages, such as the concentration of nucleic acid necessary to affect a therapeutic treatment is low. This advantage reflects the ability of the enzymatic nucleic acid molecule to act enzymatically. Thus, a single enzymatic nucleic acid molecule is able to cleave many molecules of target RNA. In addition, the enzymatic nucleic acid molecule is a highly specific modulator, with the specificity of modulation depending not only on the base-pairing mechanism of binding to the target RNA, but also on the mechanism of target RNA cleavage. Single mismatches, or base-substitutions, near the site of cleavage can be chosen to completely eliminate catalytic activity of an enzymatic nucleic acid molecule.

Nucleic acid molecules having an endonuclease enzymatic activity are able to repeatedly cleave other separate RNA molecules in a nucleotide base sequence-specific manner. With proper design and construction, such enzymatic nucleic acid molecules can be targeted to any RNA transcript, and efficient cleavage achieved in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Chartrand et al., 1995, Nucleic Acids Research 23, 4092; Santoro et al., 1997, PNAS 94, 4262).

Because of their sequence specificity, trans-cleaving enzymatic nucleic acid molecules show promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecule can be designed to cleave specific RNA targets within the background of cellular RNA. Such a cleavage event renders the RNA non-functional and abrogates protein expression from that RNA. In this manner, synthesis of a protein associated with a disease state can be selectively modulated (Warashina et al., 1999, Chemistry and Biology, 6, 237-250.

The present invention also features nucleic acid sensor molecules or allozymes having sensor domains comprising nucleic acid decoys and/or aptamers of the invention. Interaction of the nucleic acid sensor molecule's sensor domain with a molecular target, such as HCV or HBV target, e.g., HBV RT and/or HBV RT primer, can activate or inactivate the enzymatic nucleic acid domain of the nucleic acid sensor molecule, such that the activity of the nucleic acid sensor molecule is modulated in the presence of the target-signaling molecule. The nucleic acid sensor molecule can be designed to be active in the presence of the target molecule or alternately, can be designed to be inactive in the presence of the molecular target. For example, a nucleic acid sensor molecule is designed with a sensor domain having the sequence (UUCA)ₙ, where n is an integer from 1-10. In a non-limiting example, interaction of the HBV RT primer with the sensor domain of the nucleic acid sensor molecule can activate the enzymatic nucleic acid domain of the nucleic acid sensor molecule such that the sensor molecule catalyzes a reaction, for example cleavage of HBV RNA. In this example, the nucleic acid sensor molecule is activated in the presence of HBV RT or HBV RT primer, and can be used as a therapeutic to treat HBV infection. Alternately, the reaction can comprise cleavage or ligation of a labeled nucleic acid reporter molecule, providing a useful diagnostic reagent to detect the presence of HBV in a system.

HCV Target Sites

Targets for useful nucleic acid molecules and nuclease activating compounds or chimeras can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468. Rather than repeat the guidance provided in those documents here, below are provided specific examples of such methods, not limiting to those in the art. Nucleic acid molecules and nuclease activating compounds or chimeras to such targets are designed as described in those applications and synthesized to be tested in vitro and in vivo, as also described. Such nucleic acid molecules and nuclease activating compounds or chimeras can also be optimized and delivered as described therein.

The sequence of HCV RNAs were screened for optimal enzymatic nucleic acid molecule target sites using a computer folding algorithm. Enzymatic nucleic acid cleavage sites were identified. These sites are shown in Tables XVIII, XIX, XX and XXIII (All sequences are 5′ to 3′ in the tables). The nucleotide base position is noted in the tables as that site to be cleaved by the designated type of enzymatic nucleic acid molecule. The nucleotide base position is noted in the tables as that site to be cleaved by the designated type of enzymatic nucleic acid molecule.

Because HCV RNAs are highly homologous in certain regions, some enzymatic nucleic acid molecule target sites are also homologous. In this case, a single enzymatic nucleic acid molecule will target different classes of HCV RNA. The advantage of one enzymatic nucleic acid molecule that targets several classes of HCV RNA is clear, especially in cases where one or more of these RNAs can contribute to the disease state.

Enzymatic nucleic acid molecules were designed that could bind and were individually analyzed by computer folding (Jaeger et al., 1989 Proc. Natl. Acad. Sci. USA, 86, 7706) to assess whether the enzymatic nucleic acid molecule sequences fold into the appropriate secondary structure. Those enzymatic nucleic acid molecules with unfavorable intramolecular interactions between the binding arms and the catalytic core are eliminated from consideration. Varying binding arm lengths can be chosen to optimize activity. Generally, at least 5 bases on each arm are able to bind to, or otherwise interact with, the target RNA. Enzymatic nucleic acid molecules were designed to anneal to various sites in the mRNA message. The binding arms are complementary to the target site sequences described above.

HBV Target Sites

Targets for useful ribozymes and antisense nucleic acids targeting HBV can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468. Other examples include the following PCT applications, which concern inactivation of expression of disease-related genes: WO 95/23225, WO 95/13380, WO 94/02595. Rather than repeat the guidance provided in those documents here, below are provided specific examples of such methods, not limiting to those in the art. Ribozymes and antisense to such targets are designed as described in those applications and synthesized to be tested in vitro and in vivo, as also described. The sequence of human HBV RNAs (for example, accession AF100308.1; HBV strain 2-18; additionally, other HBV strains can be screened by one skilled in the art, see Table III for other possible strains) were screened for optimal enzymatic nucleic acid and antisense target sites using a computer-folding algorithm. Antisense, hammerhead, DNAzyme, NCH (Inozyme), amberzyme, zinzyme or G-Cleaver ribozyme binding/cleavage sites were identified. These sites are shown in Tables V to XI (all sequences are 5′ to 3′ in the tables; X can be any base-paired sequence, the actual sequence is not relevant here). The nucleotide base position is noted in the Tables as that site to be cleaved by the designated type of enzymatic nucleic acid molecule. Table IV shows substrate positions selected from Renbo et al., 1987, Sci. Sin., 30, 507, used in Draper, USSN (07/882,712), filed May 14, 1992, entitled “METHOD AND REAGENT FOR INHIBITING HEPATITIS B VIRUS REPLICATION” and Draper et al., International PCT publication No. WO 93/23569, filed Apr. 29, 1993, entitled “METHOD AND REAGENT FOR INHIBITING VIRAL REPLICATION”. While human sequences can be screened and enzymatic nucleic acid molecule and/or antisense thereafter designed, as discussed in Stinchcomb et al., WO 95/23225, mouse targeted ribozymes can be useful to test efficacy of action of the enzymatic nucleic acid molecule and/or antisense prior to testing in humans.

Antisense, hammerhead, DNAzyme, NCH (Inozyme), amberzyme, zinzyme or G-Cleaver ribozyme binding/cleavage sites were identified, as discussed above. The nucleic acid molecules were individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions such as between the binding arms and the catalytic core were eliminated from consideration. Varying binding arm lengths can be chosen to optimize activity.

Antisense, hammerhead, DNAzyme, NCH, amberzyme, zinzyme or G-Cleaver ribozyme binding/cleavage sites were identified and were designed to anneal to various sites in the RNA target. The binding arms are complementary to the target site sequences described above. The nucleic acid molecules were chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; Wincott et al., 1995 Nucleic Acids Res. 23, 2677-2684; and Caruthers et al., 1992, Methods in Enzymology 211,3-19.

Synthesis of Nucleic acid Molecules

Synthesis of nucleic acids greater than 100 nucleotides in length is difficult using automated methods, and the therapeutic cost of such molecules is prohibitive. In this invention, small nucleic acid motifs (“small” refers to nucleic acid motifs no more than 100 nucleotides in length, preferably no more than 80 nucleotides in length, and most preferably no more than 50 nucleotides in length; e.g., decoy nucleic acid molecules, aptamer nucleic acid molecules antisense nucleic acid molecules, enzymatic nucleic acid molecules) are preferably used for exogenous delivery. The simple structure of these molecules increases the ability of the nucleic acid to invade targeted regions of protein and/or RNA structure. Exemplary molecules of the instant invention are chemically synthesized, and others can similarly be synthesized.

Oligonucleotides (e.g., DNA oligonucleotides) are synthesized using protocols known in the art, for example as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table II outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM 12, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the DNA-based oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H₂O/3:1:1, vortexed and the supernatant is then added to the first supernatant. The combined supernatants, containing the oligoribonucleotide, are dried to a white powder.

The method of synthesis used for normal RNA including certain decoy nucleic acid molecules and enzymatic nucleic acid molecules follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM 12, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H₂O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA-3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA•3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive hammerhead ribozymes or binding attenuated control (BAC) oligonucleotides are synthesized by substituting a U for G₅ and a U for A₁₄ (numbering from Hertel, K. J., et al., 1992, Nucleic Acids Res., 20, 3252). Similarly, one or more nucleotide substitutions can be introduced in other nucleic acid decoy molecules to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96-well format, all that is important is the ratio of chemicals used in the reaction.

Alternatively, the nucleic acid molecules of the present invention can be synthesized separately and joined together post-synthetically, for example, by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention can be modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Ribozymes can be purified by gel electrophoresis using general methods or can be purified by high pressure liquid chromatography (HPLC; see Wincott et al., supra, the totality of which is hereby incorporated herein by reference) and re-suspended in water.

The sequences of the nucleic acid molecules that are chemically synthesized, useful in this study, are shown in Tables XI, XV, XX, XXI, XXII and XXIII. The nucleic acid sequences listed in Tables IV-XI, XIV-XV and XVIII-XXIII can be formed of ribonucleotides or other nucleotides or non-nucleotides. Such nucleic acid sequences are equivalent to the sequences described specifically in the Tables.

Optimizing Activity of the Nucleic Acid Molecule of the Invention

Chemically synthesizing nucleic acid molecules with modifications (base, sugar and/or phosphate) can prevent their degradation by serum ribonucleases, which can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; Gold et al., U.S. Pat. No. 6,300,074; and Burgin et al., supra; all of which are incorporated by reference herein). All of the above references describe various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. S No. 60/082,404 which was filed on Apr. 20, 1998; Karpeisky et al., 1998, Tetrahedron Lett., 39, 1131; Earnshaw and Gait, 1998, Biopolymers (Nucleic Acid Sciences), 48, 39-55; Verma and Eckstein, 1998, Annu. Rev. Biochem., 67, 99-134; and Burlina et al, 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into ribozymes without modulating catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, excessive modifications can cause some toxicity. Therefore, when designing nucleic acid molecules, the amount of these internucleotide linkages should be minimized. The reduction in the concentration of these linkages should lower toxicity, resulting in increased efficacy and higher specificity of these molecules.

Nucleic acid molecules having chemical modifications that maintain or enhance activity are provided. Such a nucleic acid is also generally more resistant to nucleases than an unmodified nucleic acid. Accordingly, the in vitro and/or in vivo activity should not be significantly lowered. In cases in which modulation is the goal, therapeutic nucleic acid molecules delivered exogenously should optimally be stable within cells until translation of the target RNA has been modulated long enough to reduce the levels of the undesirable protein. This period of time varies between hours to days depending upon the disease state. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein)) have expanded the ability to modify nucleic acid molecules by introducing nucleotide modifications to enhance their nuclease stability, as described above.

In one embodiment, nucleic acid molecules of the invention include one or more G-clamp nucleotides. A G-clamp nucleotide is a modified cytosine analog wherein the modifications confer the ability to hydrogen bond both Watson-Crick and Hoogsteen faces of a complementary guanine within a duplex, see for example Lin and Matteucci, 1998, J. Am. Chem. Soc., 120, 8531-8532. A single G-clamp analog substation within an oligonucleotide can result in substantially enhanced helical thermal stability and mismatch discrimination when hybridized to complementary oligonucleotides. The inclusion of such nucleotides in nucleic acid molecules of the invention results in both enhanced affinity and specificity to nucleic acid targets. In another embodiment, nucleic acid molecules of the invention include one or more LNA “locked nucleic acid” nucleotides such as a 2′,4′-C methylene bicyclo nucleotide (see for example Wengel et al., International PCT Publication No. WO 00/66604 and WO 99/14226).

In another embodiment, the invention features conjugates and/or complexes of nucleic acid molecules targeting HBV or HCV. Such conjugates and/or complexes can be used to facilitate delivery of molecules into a biological system, such as a cell. The conjugates and complexes provided by the instant invention can impart therapeutic activity by transferring therapeutic compounds across cellular membranes, altering the pharmacokinetics, and/or modulating the localization of nucleic acid molecules of the invention. The present invention encompasses the design and synthesis of novel conjugates and complexes for the delivery of molecules, including, but not limited to, small molecules, lipids, phospholipids, nucleosides, nucleotides, nucleic acids, antibodies, toxins, negatively charged polymers and other polymers, for example proteins, peptides, hormones, carbohydrates, polyethylene glycols, or polyamines, across cellular membranes. In general, the transporters described are designed to be used either individually or as part of a multi-component system, with or without degradable linkers. These compounds are expected to improve delivery and/or localization of nucleic acid molecules of the invention into a number of cell types originating from different tissues, in the presence or absence of serum (see Sullenger and Cech, U.S. Pat. No. 5,854,038). Conjugates of the molecules described herein can be attached to biologically active molecules via linkers that are biodegradable, such as biodegradable nucleic acid linker molecules.

The term “biodegradable nucleic acid linker molecule” as used herein, refers to a nucleic acid molecule that is designed as a biodegradable linker to connect one molecule to another molecule, for example, a biologically active molecule. The stability of the biodegradable nucleic acid linker molecule can be modulated by using various combinations of ribonucleotides, deoxyribonucleotides, and chemically modified nucleotides, for example, 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus-based linkage, for example, a phosphoramidate or phosphodiester linkage. The biodegradable nucleic acid linker molecule can also comprise nucleic acid backbone, nucleic acid sugar, or nucleic acid base modifications.

The term “biodegradable” as used herein, refers to degradation in a biological system, for example enzymatic degradation or chemical degradation.

The term “biologically active molecule” as used herein, refers to compounds or molecules that are capable of eliciting or modifying a biological response in a system. Non-limiting examples of biologically active molecules contemplated by the instant invention include therapeutically active molecules such as antibodies, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siRNA, dsRNA, allozymes, aptamers, decoys and analogs thereof. Biologically active molecules of the invention also include molecules capable of modulating the pharmacokinetics and/or pharmacodynamics of other biologically active molecules, for example, lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus-containing group and saturated or unsaturated alkyl group, optionally substituted with OH, COOH, oxo, amine, or substituted or unsubstituted aryl groups.

Therapeutic nucleic acid molecules (e.g., decoy nucleic acid molecules) delivered exogenously optimally are stable within cells until reverse trascription of the pregenomic RNA has been modulated long enough to reduce the levels of HBV or HCV DNA. The nucleic acid molecules are resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

In yet another embodiment, nucleic acid molecules having chemical modifications that maintain or enhance enzymatic activity are provided. Such nucleic acids are also generally more resistant to nucleases than unmodified nucleic acids. Thus, in vitro and/or in vivo the activity should not be significantly lowered. As exemplified herein, such nucleic acid molecules are useful in vitro and/or in vivo even if activity over all is reduced 10 fold (Burgin et al., 1996, Biochemistry, 35, 14090).

Use of the nucleic acid-based molecules of the invention will lead to better treatment of the disease progression by affording the possibility of combination therapies (e.g., multiple antisense, nucleic acid decoy, or nucleic acid aptamer molecules targeted to different genes; nucleic acid molecules coupled with known small molecule modulators ors; or intermittent treatment with combinations of molecules (including different motifs) and/or other chemical or biological molecules). The treatment of patients with nucleic acid molecules may also include combinations of different types of nucleic acid molecules.

In another aspect the nucleic acid molecules comprise a 5′ and/or a 3′-cap structure.

By “cap structure” is meant chemical modifications, which have been incorporated at either terminus of the oligonucleotide (see, for example, Wincott et al., WO 97/26270, incorporated by reference herein). These terminal modifications protect the nucleic acid molecule from exonuclease degradation, and may help in delivery and/or localization within a cell. The cap may be present at the 5′-terminus (5′-cap) or at the 3′-terminal (3′-cap) or may be present on both termini. In non-limiting examples: the 5′-cap is selected from the group comprising inverted abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details, see Wincott et al., International PCT publication No. WO 97/26270, incorporated by reference herein).

In yet another preferred embodiment, the 3′-cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

The term “alkyl” as used herein refers to a saturated aliphatic hydrocarbon, including straight-chain, branched-chain “isoalkyl”, and cyclic alkyl groups. The term “alkyl” also comprises alkoxy, alkyl-thio, alkyl-thio-alkyl, alkoxyalkyl, alkylamino, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. Preferably, the alkyl group has 1 to 12 carbons. More preferably it is a lower alkyl of from about 1 to 7 carbons, more preferably about 1 to 4 carbons. The alkyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. The term “alkyl” also includes alkenyl groups containing at least one carbon-carbon double bond, including straight-chain, branched-chain, and cyclic groups. Preferably, the alkenyl group has about 2 to 12 carbons. More preferably it is a lower alkenyl of from about 2 to 7 carbons, more preferably about 2 to 4 carbons. The alkenyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. The term “alkyl” also includes alkynyl groups containing at least one carbon-carbon triple bond, including straight-chain, branched-chain, and cyclic groups. Preferably, the alkynyl group has about 2 to 12 carbons. More preferably it is a lower alkynyl of from about 2 to 7 carbons, more preferably about 2 to 4 carbons. The alkynyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. Alkyl groups or moieties of the invention can also include aryl, alkylaryl, carbocyclic aryl, heterocyclic aryl, amide and ester groups. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from about 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

The term “alkoxyalkyl” as used herein refers to an alkyl-O-alkyl ether, for example methoxyethyl or ethoxymethyl.

The term “alkyl-thio-alkyl” as used herein refers to an alkyl-S-alkyl thioether, for example methylthiomethyl or methylthioethyl.

The term “amination” as used herein refers to a process in which an amino group or substituted amine is introduced into an organic molecule.

The term “exocyclic amine protecting moiety” as used herein refers to a nucleobase amino protecting group compatible with oligonucleotide synthesis, for example an acyl or amide group.

The term “alkenyl” as used herein refers to a straight or branched hydrocarbon of a designed number of carbon atoms containing at least one carbon-carbon double bond. Examples of “alkenyl” include vinyl, allyl, and 2-methyl-3-heptene.

The term “alkoxy” as used herein refers to an alkyl group of indicated number of carbon atoms attached to the parent molecular moiety through an oxygen bridge. Examples of alkoxy groups include, for example, methoxy, ethoxy, propoxy and isopropoxy.

The term “alkynyl” as used herein refers to a straight or branched hydrocarbon of a designed number of carbon atoms containing at least one carbon-carbon triple bond. Examples of “alkynyl” include propargyl, propyne, and 3-hexyne.

The term “aryl” as used herein refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

The term “cycloalkenyl” as used herein refers to a C3-C8 cyclic hydrocarbon containing at least one carbon-carbon double bond. Examples of cycloalkenyl include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “cycloalkyl” as used herein refers to a C3-C8 cyclic hydrocarbon. Examples of cycloalkyl include cyclopropyl, cyclobutyl, cyclopentyl, cyclohexyl, cycloheptyl and cyclooctyl.

The term “cycloalkylalkyl,” as used herein, refers to a C3-C7 cycloalkyl group attached to the parent molecular moiety through an alkyl group, as defined above. Examples of cycloalkylalkyl groups include cyclopropylmethyl and cyclopentylethyl.

The terms “halogen” or “halo” as used herein refers to indicate fluorine, chlorine, bromine, and iodine.

The term “heterocycloalkyl,” as used herein refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrolidinyl.

The term “heteroaryl” as used herein refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

The term “C1-C6 hydrocarbyl” as used herein refers to straight, branched, or cyclic alkyl groups having 1-6 carbon atoms, optionally containing one or more carbon-carbon double or triple bonds. Examples of hydrocarbyl groups include, for example, methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, vinyl, 2-pentene, cyclopropylmethyl, cyclopropyl, cyclohexylmethyl, cyclohexyl and propargyl. When reference is made herein to C1-C6 hydrocarbyl containing one or two double or triple bonds it is understood that at least two carbons are present in the alkyl for one double or triple bond, and at least four carbons for two double or triple bonds.

The term “nucleotide” as used herein refers to a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein. There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, for example, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N-6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

The term “nucleoside” as used herein refers to a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N-6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

In one embodiment, the invention features modified nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

The term “abasic” as used herein refers to sugar moieties lacking a base or having other chemical groups in place of a base at the 1′ position, for example a 3′,3′-linked or 5′,5′-linked deoxyabasic ribose derivative (for more details see Wincott et al., International PCT publication No. WO 97/26270).

The term “unmodified nucleoside” as used herein refers to one of the bases adenine, cytosine, guanine, thymine, uracil joined to the 1′ carbon of β-D-ribo-furanose.

The term “modified nucleoside” as used herein refers to any nucleotide base which contains a modification in the chemical structure of an unmodified nucleotide base, sugar and/or phosphate.

In connection with 2′-modified nucleotides as described for the present invention, by “amino” is meant 2′-NH₂ or 2′-O—NH₂, which can be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid (e.g., enzymatic nucleic acid, antisense, decoy, aptamer, siRNA, triplex oligonucleotides, 2,5-A oligonucleotides and other nucleic acid molecules) structure can be made to enhance the utility of these molecules. For example, such modifications can enhance shelf life, half-life in vitro, stability, and ease of introduction of such oligonucleotides to the target site, including e.g., enhancing penetration of cellular membranes and conferring the ability to recognize and bind to targeted cells.

Use of these molecules can lead to better treatment of the disease progression by affording the possibility of combination therapies (e.g., multiple nucleic acid molecules targeted to different genes, nucleic acid molecules coupled with known small molecule inhibitors, or intermittent treatment with combinations of nucleic acid molecules (including different nucleic acid molecule motifs) and/or other chemical or biological molecules). The treatment of patients with nucleic acid molecules can also include combinations of different types of nucleic acid molecules. Therapies can be devised which include a mixture of enzymatic nucleic acid molecules (including different enzymatic nucleic acid molecule motifs), antisense, decoy, aptamer and/or 2-5A chimera molecules to one or more targets to alleviate symptoms of a disease.

Administration of Nucleic Acid Molecules

Methods for the delivery of nucleic acid molecules are described in Akhtar et al., 1992, Trends Cell Bio., 2, 139; Delivery Strategies for Antisense Oligonucleotide Therapeutics, ed. Akhtar, 1995, Maurer et al., 1999, Mol. Membr. Biol., 16, 129-140; Hofland and Huang, 1999, Handb. Exp. Pharmacol., 137, 165-192; and Lee et al., 2000, ACS Symp. Ser., 752, 184-192, Sullivan et al., PCT WO 94/02595, further describes the general methods for delivery of enzymatic nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as hydrogels, cyclodextrins, biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., 1999, Clin. Cancer Res., 5, 2330-2337 and Barry et al., International PCT Publication No. WO 99/31262. The molecules of the instant invention can be used as pharmaceutical agents. Pharmaceutical agents prevent, modulate the occurrence, or treat (alleviate a symptom to some extent, preferably all of the symptoms) of a disease state in a patient.

Thus, the invention features a pharmaceutical composition comprising one or more nucleic acid(s) of the invention in an acceptable carrier, such as a stabilizer, buffer, and the like. The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention may also be formulated and used as tablets, capsules or elixirs for oral administration, suppositories for rectal administration, sterile solutions, suspensions for injectable administration, and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, including for example a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity and forms that prevent the composition or formulation from exerting its effect.

By “systemic administration” is meant in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

By “pharmaceutically acceptable formulation” is meant, a composition or formulation that allows for the effective distribution of the nucleic acid molecules of the instant invention in the physical location most suitable for their desired activity. Nonlimiting examples of agents suitable for formulation with the nucleic acid molecules of the instant invention include: P-glycoprotein inhibitors (such as Pluronic P85), which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich, D F et al, 1999, Cell Transplant, 8, 47-58) (Alkermes, Inc. Cambridge, Mass.); and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies for the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

The present invention also includes compositions prepared for storage or administration, which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents may be provided. These include sodium benzoate, sorbic acid and esters of p-hydroxybenzoic acid. In addition, antioxidants and suspending agents may be used.

A pharmaceutically effective dose is that dose required to prevent, inhibit the occurrence of, or treat (alleviate a symptom to some extent, preferably all of the symptoms) a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

The present invention also includes compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention may also be administered to a patient in combination with other therapeutic compounds to increase the overall therapeutic effect. The use of multiple compounds to treat an indication may increase the beneficial effects while reducing the presence of side effects.

In one embodiment, the invention compositions suitable for administering nucleic acid molecules of the invention to specific cell types, such as hepatocytes. For example, the asialoglycoprotein receptor (ASGPr) (Wu and Wu, 1987, J. Biol. Chem. 262, 4429-4432) is unique to hepatocytes and binds branched galactose-terminal glycoproteins, such as asialoorosomucoid (ASOR). Binding of such glycoproteins or synthetic glycoconjugates to the receptor takes place with an affinity that strongly depends on the degree of branching of the oligosaccharide chain, for example, triatennary structures are bound with greater affinity than biatenarry or monoatennary chains (Baenziger and Fiete, 1980, Cell, 22, 611-620; Connolly et al., 1982, J. Biol. Chem., 257, 939-945). Lee and Lee, 1987, Glycoconjugate J., 4, 317-328, obtained this high specificity through the use of N-acetyl-D-galactosamine as the carbohydrate moiety, which has higher affinity for the receptor, compared to galactose. This “clustering effect” has also been described for the binding and uptake of mannosyl-terminating glycoproteins or glycoconjugates (Ponpipom et al., 1981, J. Med. Chem., 24, 1388-1395). The use of galactose and galactosamine based conjugates to transport exogenous compounds across cell membranes can provide a targeted delivery approach to the treatment of liver disease such as HBV infection or hepatocellular carcinoma. The use of bioconjugates can also provide a reduction in the required dose of therapeutic compounds required for treatment. Furthermore, therapeutic bioavialability, pharmacodynamics, and pharmacokinetic parameters can be modulated through the use of nucleic acid bioconjugates of the invention.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector. The activity of such nucleic acids can be augmented by their release from the primary transcript by a ribozyme (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totality by reference herein).

In another aspect of the invention, RNA molecules of the present invention are preferably expressed from transcription units (see, for example, Couture et al., 1996, TIG., 12, 510) inserted into DNA or RNA vectors. The recombinant vectors are preferably DNA plasmids or viral vectors. Ribozyme expressing viral vectors could be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors may be used that provide for transient expression of nucleic acid molecules. Such vectors might be repeatedly administered as necessary. Once expressed, the nucleic acid molecule binds to the target mRNA. Delivery of nucleic acid molecule expressing vectors could be systemic, such as by intravenous or intra-muscular administration, by administration to target cells ex-planted from the patient followed by reintroduction into the patient, or by any other means that would allow for introduction into the desired target cell (for a review see Couture et al., 1996, TIG., 12, 510).

In one aspect, the invention features an expression vector comprising a nucleic acid sequence encoding at least one of the nucleic acid molecules of the instant invention is disclosed. The nucleic acid sequence encoding the nucleic acid molecule of the instant invention is operable linked in a manner which allows expression of that nucleic acid molecule.

In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector may optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. USA, 89, 10802-6; Chen et al., 1992, Nucleic Acids Res., 20, 4581-9; Yu et al., 1993, Proc. Natl. Acad. Sci. USA, 90, 6340-4; L'Huillier et al., 1992, EMBO J., 11, 4411-8; Lisziewicz et al., 1993, Proc. Natl. Acad. Sci. U.S.A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736; all of these publications are incorporated by reference herein). The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In yet another aspect, the invention features an expression vector comprising nucleic acid sequence encoding at least one of the nucleic acid molecules of the invention, in a manner that allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule. In another embodiment, the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment, the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule. In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

Interferons

Type I interferons (IFN) are a class of natural cytokines that includes a family of greater than 25 IFN-α (Pesta, 1986, Methods Enzymol. 119, 3-14) as well as IFN-β, and IFN-ω. Although evolutionarily derived from the same gene (Diaz et al., 1994, Genomics 22, 540-552), there are many differences in the primary sequence of these molecules, implying an evolutionary divergence in biologic activity. All type I IFN share a common pattern of biologic effects that begin with binding of the IFN to the cell surface receptor (Pfeffer & Strulovici, 1992, Transmembrane secondary messengers for IFN-α/p. In: Interferon. Principles and Medical Applications., S. Baron, D. H. Coopenhaver, F. Dianzani, W. R. Fleischmann Jr., T. K. Hughes Jr., G. R. Kimpel, D. W. Niesel, G. J. Stanton, and S. K. Tyring, eds. 151-160). Binding is followed by activation of tyrosine kinases, including the Janus tyrosine kinases and the STAT proteins, which leads to the production of several IFN-stimulated gene products (Johnson et al., 1994, Sci. Am. 270, 68-75). The IFN-stimulated gene products are responsible for the pleotropic biologic effects of type I IFN, including antiviral, antiproliferative, and immunomodulatory effects, cytokine induction, and HLA class I and class II regulation (Pestka et al., 1987, Annu. Rev. Biochem 56, 727). Examples of IFN-stimulated gene products include 2-5-oligoadenylate synthetase (2-5 OAS), β₂-microglobulin, neopterin, p68 kinases, and the Mx protein (Chebath & Revel, 1992 The 2-5 A system: 2-5 A synthetase, isospecies and functions. In: Interferon. Principles and Medical Applications. S. Baron, D. H. Coopenhaver, F. Dianzani, W. R. Jr. Fleischmann, T. K. Jr Hughes, G. R. Kimpel, D. W. Niesel, G. J. Stanton, and S. K. Tyring, eds., pp. 225-236; Samuel, 1992, The RNA-dependent P1/eIF-2α protein kinase. In: Interferon. Principles and Medical Applications. S. Baron, D. H. Coopenhaver, F. Dianzani, W. R. Fleischmann Jr., T. K. Hughes Jr., G. R. Kimpel, D. W. Niesel, G. H. Stanton, and S. K. Tyring, eds. 237-250; Horisberger, 1992, MX protein: function and Mechanism of Action. In: Interferon. Principles and Medical Applications. S. Baron, D. H. Coopenhaver, F. Dianzani, W. R. Fleischmann Jr., T. K. Hughes Jr., G. R. Kimpel, D. W. Niesel, G. H. Stanton, and S. K. Tyring, eds. 215-224). Although all type I IFN have similar biologic effects, not all the activities are shared by each type I IFN, and, in many cases, the extent of activity varies quite substantially for each IFN subtype (Fish et al, 1989, J. Interferon Res. 9, 97-114; Ozes et al., 1992, J. Interferon Res. 12, 55-59). More specifically, investigations into the properties of different subtypes of IFN-α and molecular hybrids of IFN-α have shown differences in pharmacologic properties (Rubinstein, 1987, J. Interferon Res. 7, 545-551). These pharmacologic differences can arise from as few as three amino acid residue changes (Lee et al., 1982, Cancer Res. 42, 1312-1316).

Eighty-five to 166 amino acids are conserved in the known IFN-α subtypes. Excluding the IFN-α pseudogenes, there are approximately 25 known distinct IFN-α subtypes. Pairwise comparisons of these nonallelic subtypes show primary sequence differences ranging from 2% to 23%. In addition to the naturally occurring IFNs, a non-natural recombinant type I interferon known as consensus interferon (CIFN) has been synthesized as a therapeutic compound (Tong et al., 1997, Hepatology 26, 747-754).

Interferon is currently in use for at least 12 different indications including infectious and autoimmune diseases and cancer (Borden, 1992, N. Engl. J. Med. 326, 1491-1492). For autoimmune diseases IFN has been utilized for treatment of rheumatoid arthritis, multiple sclerosis, and Crohn's disease. For treatment of cancer IFN has been used alone or in combination with a number of different compounds. Specific types of cancers for which IFN has been used include squamous cell carcinomas, melanomas, hypernephromas, hemangiomas, hairy cell leukemia, and Kaposi's sarcoma. In the treatment of infectious diseases, IFNs increase the phagocytic activity of macrophages and cytotoxicity of lymphocytes and inhibits the propagation of cellular pathogens. Specific indications for which IFN has been used as treatment include: hepatitis B, human papillomavirus types 6 and 11 (i.e. genital warts) (Leventhal et al., 1991, N Engl J Med 325, 613-617), chronic granulomatous disease, and hepatitis C virus.

Numerous well controlled clinical trials using IFN-alpha in the treatment of chronic HCV infection have demonstrated that treatment three times a week results in lowering of serum ALT values in approximately 50% (range 40% to 70%) of patients by the end of 6 months of therapy (Davis et al., 1989, The new England Journal of Medicine 321, 1501-1506; Marcellin et al., 1991, Hepatology 13, 393-397; Tong et al., 1997, Hepatology 26, 747-754; Tong et al., Hepatology 26, 1640-1645). However, following cessation of interferon treatment, approximately 50% of the responding patients relapsed, resulting in a “durable” response rate as assessed by normalization of serum ALT concentrations of approximately 20 to 25%. In addition, studies that have examined six months of type 1 interferon therapy using changes in HCV RNA values as a clinical endpoint have demonstrated that up to 35% of patients will have a loss of HCV RNA by the end of therapy (Tong et al., 1997, supra). However, as with the ALT endpoint, about 50% of the patients relapse six months following cessation of therapy resulting in a durable virologic response of only 12% (23). Studies that have examined 48 weeks of therapy have demonstrated that the sustained virological response is up to 25%.

Pegylated interferons, ie. interferons conjugated with polyethylene glycol (PEG), have demonstrated improved characteristics over interferon. Advantages incurred by PEG conjugation can include an improved pharmacokinetic profile compared to interferons lacking PEG, thus imparting more convenient dosing regimes, improved tolerance, and improved antiviral efficacy. Such improvements have been demonstrated in clinical studies of both polyethylene glycol interferon alfa-2a (PEGASYS, Roche) and polyethylene glycol interferon alfa-2b (VIRAFERON PEG, PEG-INTRON, Enzon/Schering Plough).

Enzymatic nucleic acid molecules in combination with interferons and polyethylene glycol interferons have the potential to improve the effectiveness of treatment of HCV or any of the other indications discussed above. Enzymatic nucleic acid molecules targeting RNAs associated with diseases such as infectious diseases, autoimmune diseases, and cancer, can be used individually or in combination with other therapies such as interferons and polyethylene glycol interferons and to achieve enhanced efficacy.

### EXAMPLES

The following are non-limiting examples showing the selection, isolation, synthesis and activity of nucleic acids of the instant invention. These examples demonstrate the selection and design of Antisense, Hammerhead, DNAzyme, NCH, Amberzyme, Zinzyme or G-Cleaver ribozyme molecules and binding/cleavage sites within HBV and HCV RNA. The following examples also demonstrate the selection and design of nucleic acid decoy molecules that target HBV reverse transcriptase. The following examples also demonstrate the use of enzymatic nucleic acid molecules that cleave HCV RNA. The methods described herein represent a scheme by which nucleic acid molecules can be derived that cleave other RNA targets required for HCV replication.

### Example 1

### Identification of Potential Target Sites in Human HBV RNA

The sequence of human HBV was screened for accessible sites using a computer-folding algorithm. Regions of the RNA that did not form secondary folding structures and contained potential ribozyme and/or antisense binding/cleavage sites were identified. The sequences of these cleavage sites are shown in Tables IV-XI.

### Example 2

### Selection of Enzymatic Nucleic Acid Cleavage Sites in Human HBV RNA

Ribozyme target sites were chosen by analyzing sequences of Human HBV (accession number: AF100308.1) and prioritizing the sites on the basis of folding. Ribozymes were designed that could bind each target and were individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the ribozyme sequences fold into the appropriate secondary structure. Those ribozymes with unfavorable intramolecular interactions between the binding arms and the catalytic core were eliminated from consideration. As noted herein, varying binding arm lengths can be chosen to optimize activity. Generally, at least 5 bases on each arm are able to bind to, or otherwise interact with, the target RNA.

### Example 3

### Chemical Synthesis and Purification of Ribozymes and Antisense for Efficient Cleavage and/or Blocking of HBV RNA

Ribozymes and antisense constructs were designed to anneal to various sites in the RNA message. The binding arms of the ribozymes are complementary to the target site sequences described above, while the antisense constructs are fully complementary to the target site sequences described above. The ribozymes and antisense constructs were chemically synthesized. The method of synthesis used followed the procedure for normal RNA synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were typically >98%.

Ribozymes and antisense constructs were also synthesized from DNA templates using bacteriophage T7 RNA polymerase (Milligan and Uhlenbeck, 1989, Methods Enzymol. 180, 51). Ribozymes and antisense constructs were purified by gel electrophoresis using general methods or were purified by high pressure liquid chromatography (HPLC; see Wincott et al., supra; the totality of which is hereby incorporated herein by reference) and were resuspended in water. The sequences of the chemically synthesized ribozymes used in this study are shown below in Table XI.

### Example 4

### Ribozyme Cleavage of HBV RNA Target In Vitro

Ribozymes targeted to the human HBV RNA are designed and synthesized as described above. These ribozymes can be tested for cleavage activity in vitro, for example using the following procedure. The target sequences and the nucleotide location within the HBV RNA are given in Tables IV-XI.

Cleavage Reactions: Full-length or partially full-length, internally-labeled target RNA for ribozyme cleavage assay is prepared by in vitro transcription in the presence of [α-³²P] CTP, passed over a G 50 Sephadex® column by spin chromatography and used as substrate RNA without further purification. Alternately, substrates are 5′-³²P-end labeled using T4 polynucleotide kinase enzyme. Assays are performed by pre-warming a 2× concentration of purified ribozyme in ribozyme cleavage buffer (50 mM Tris-HCl, pH 7.5 at 37° C., 10 mM MgCl₂) and the cleavage reaction was initiated by adding the 2× ribozyme mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM ribozyme, i.e., ribozyme excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by ribozyme cleavage are visualized on an autoradiograph of the gel. The percentage of cleavage is determined by Phosphor Imager® quantitation of bands representing the intact substrate and the cleavage products.

### Example 5

### Transfection of HepG2 Cells with psHBV-1 and Ribozymes

The human hepatocellular carcinoma cell line Hep G2 was grown in Dulbecco's modified Eagle media supplemented with 10% fetal calf serum, 2 mM glutamine, 0.1 mM nonessential amino acids, 1 mM sodium pyruvate, 25 mM Hepes, 100 units penicillin, and 100 μg/ml streptomycin. To generate a replication competent cDNA, prior to transfection the HBV genomic sequences are excised from the bacterial plasmid sequence contained in the psHBV-1 vector (Those skilled in the art understand that other methods may be used to generate a replication competent cDNA). This was done with an EcoRI and Hind III restriction digest. Following completion of the digest, a ligation was performed under dilute conditions (20 μg/ml) to favor intermolecular ligation. The total ligation mixture was then concentrated using Qiagen spin columns.

Secreted alkaline phosphatase (SEAP) was used to normalize the HBsAg levels to control for transfection variability. The pSEAP2-TK control vector was constructed by ligating a Bgl II-Hind III fragment of the pRL-TK vector (Promega), containing the herpes simplex virus thymidine kinase promoter region, into Bgl II/Hind III digested pSEAP2-Basic (Clontech). Hep G2 cells were plated (3×10⁴ cells/well) in 96-well microtiter plates and incubated overnight. A lipid/DNA/ribozyme complex was formed containing (at final concentrations) cationic lipid (15 μg/ml), prepared psHBV-1 (4.5 μg/ml), pSEAP2-TK (0.5 μg/ml), and ribozyme (100 μM). Following a 15 min. incubation at 37° C., the complexes were added to the plated Hep G2 cells. Media was removed from the cells 96 hr. post-transfection for HBsAg and SEAP analysis.

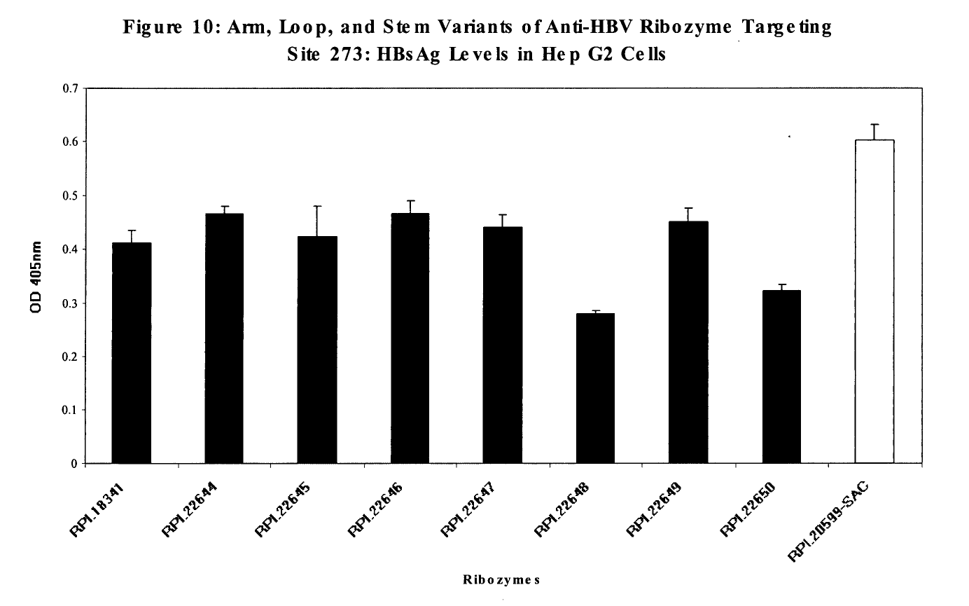

Transfection of the human hepatocellular carcinoma cell line, Hep G2, with replication competent HBV DNA results in the expression of HBV proteins and the production of virions. To investigate the potential use of ribozymes for the treatment of chronic HBV infection, a series of ribozymes that target the 3′ terminus of the HBV genome have been synthesized. Ribozymes targeting this region have the potential to cleave all four major HBV RNA transcripts as well as the potential to block the production of HBV DNA by cleavage of the pregenomic RNA. To test the efficacy of these HBV ribozymes, they were co-transfected with HBV genomic DNA into Hep G2 cells, and the subsequent levels of secreted HBV surface antigen (HBsAg) were analyzed by ELISA. To control for variability in transfection efficiency, a control vector which expresses secreted alkaline phosphatase (SEAP), was also co-transfected. The efficacy of the HBV ribozymes was determined by comparing the ratio of HBsAg:SEAP and/or HBeAg:SEAP to that of a scrambled attenuated control (SAC) ribozyme. Twenty-five ribozymes (RPI18341, RPI18356, RPI18363, RPI18364, RPI18365, RPI18366, RPI18367, RPI18368, RPI18369, RPI18370, RPI18371, RPI18372, RPI18373, RPI18374, RPI18303, RPI18405, RPI18406, RPI18407, RPI18408, RPI18409, RPI18410, RPI18411, RPI18418, RPI18419, and RPI18422) have been identified which cause a reduction in the levels of HBsAg and/or HBeAg as compared to the corresponding SAC ribozyme. In addition, loop variant anti-HBV ribozymes targeting site 273 were tested using this system, the results of this study are summarized in FIG. 10. As indicated in the figure, the ribozymes tested demonstrate significant reduction in HepG2 HBsAg levels as compared to a scrambled attenuated core ribozyme control, with RPI 22650 and RPI 22649 showing the greatest decrease in HBsAg levels.

### Example 6

### Analysis of HBsAg and SEAP Levels Following Ribozyme Treatment

Immulon 4 (Dynax) microtiter wells were coated overnight at 4° C. with anti-HBsAg Mab (Biostride B88-95-31 ad,ay) at 1 μg/ml in Carbonate Buffer (Na2CO3 15 mM, NaHCO3 35 mM, pH 9.5). The wells were then washed 4× with PBST (PBS, 0.05% Tween® 20) and blocked for 1 hr at 37° C. with PBST, 1% BSA. Following washing as above, the wells were dried at 37° C. for 30 min. Biotinylated goat ant-HBsAg (Accurate YVS1807) was diluted 1:1000 in PBST and incubated in the wells for 1 hr. at 37° C. The wells were washed 4× with PBST. Streptavidin/Alkaline Phosphatase Conjugate (Pierce 21324) was diluted to 250 ng/ml in PBST, and incubated in the wells for 1 hr. at 37° C. After washing as above, p-nitrophenyl phosphate substrate (Pierce 37620) was added to the wells, which were then incubated for 1 hr. at 37° C. The optical density at 405 nm was then determined. SEAP levels were assayed using the Great EscAPe® Detection Kit (Clontech K2041-1), as per the manufacturers instructions.

### Example 7

### X-gene Reporter Assay

The effect of ribozyme treatment on the level of transactivation of a SV40 promoter driven firefly luciferase gene by the HBV X-protein was analyzed in transfected Hep G2 cells. As a control for variability in transfection efficiency, a Renilla luciferase reporter driven by the TK promoter, which is not transactivated by the X protein, was used. Hep G2 cells were plated (3×10⁴ cells/well) in 96-well microtiter plates and incubated overnight. A lipid/DNA/ribozyme complex was formed containing (at final concentrations) cationic lipid (2.4 μg/ml), the X-gene vector pSBDR (2.5 μg/ml), the firefly reporter pSV40HCVluc (0.5 μg/ml), the Renilla luciferase control vector pRL-TK (0.5 μg/ml), and ribozyme (100 μM). Following a 15 min. incubation at 37° C., the complexes were added to the plated Hep G2 cells. Levels of firefly and Renilla luciferase were analyzed 48 hr. post transfection, using Promega's Dual-Luciferase Assay System.

The HBV X protein is a transactivator of a number of viral and cellular genes. Ribozymes which target the X region were tested for their ability to cause a reduction in X protein transactivation of a firefly luciferase gene driven by the SV40 promoter in transfected Hep G2 cells. As a control for transfection variability, a vector containing the Renilla luciferase gene driven by the TK promotor, which is not activated by the X protein, was included in the co-transfections. The efficacy of the HBV ribozymes was determined by comparing the ratio of firefly luciferase: Renilla luciferase to that of a scrambled attenuated control (SAC) ribozyme. Eleven ribozymes (RPI18365, RPI18367, RPI18368, RPI18371, RPI18372, RPI18373, RPI18405, RPI18406, RPI18411, RPI18418, RPI18423) were identified which cause a reduction in the level of transactivation of a reporter gene by the X protein, as compared to the corresponding SAC ribozyme.

### Example 8

### HBV Transgenic Mouse Study A

A transgenic mouse strain (founder strain 1.3.32 with a C57B1/6 background) that expresses HBV RNA and forms HBV viremia (Morrey et al., 1999, Antiviral Res., 42, 97-108; Guidotti et al., 1995, J. Virology, 69, 10, 6158-6169) was utilized to study the in vivo activity of ribozymes (RPI.18341, RPI.18371, RPI.18372, and RPI.18418) of the instant invention. This model is predictive in screening for anti-HBV agents. Ribozyme or the equivalent volume of saline was administered via a continuous s.c. infusion using Alzet® mini-osmotic pumps for 14 days. Alzet® pumps were filled with test material(s) in a sterile fashion according to the manufacturer's instructions. Prior to in vivo implantation, pumps were incubated at 37° C. overnight (≧18 hours) to prime the flow modulators. On the day of surgery, animals were lightly anesthetized with a ketamine/xylazine cocktail (94 mg/kg and 6 mg/kg, respectively; 0.3 ml, IP). Baseline blood samples (200 μl) were obtained from each animal via a retro-orbital bleed. For animals in groups 1-5 (Table XII), a 2 cm area near the base of the tail was shaved and cleansed with betadine surgical scrub and sequentially with 70% alcohol. A 1 cm incision in the skin was made with a #15 scalpel blade or a blunt pair of scissors near the base of the tail. Forceps were used to open a pocket rostrally (ie., towards the head) by spreading apart the subcutaneous connective tissue. The pump was inserted with the delivery portal pointing away from the incision. Wounds were closed with sterile 9-mm stainless steel clips or with sterile 4-0 suture. Animals were then allowed to recover from anesthesia on a warm heating pad before being returned to their cage. Wounds were checked daily. Clips or sutures were replaced as needed. Incisions typically healed completely within 7 days post-op. Animals were then deeply anesthetized with the ketamine/xylazine cocktail (150 mg/kg and 10 mg/kg, respectively; 0.5 ml, IP) on day 14 post pump implantation. A midline thoracotomy/laparatomy was performed to expose the abdominal cavity and the thoracic cavity. The left ventricle was cannulated at the base and animals exsanguinated using a 23G needle and 1 ml syringe. Serum was separated, frozen and analyzed for HBV DNA and antigen levels. Experimental groups were compared to the saline control group in respect to percent change from day 0 to day 14. HBV DNA was assayed by quantitative PCR.

Results

Table XII is a summary of the group designation and dosage levels used in this HBV transgenic mouse study. Baseline blood samples were obtained via a retroorbital bleed and animals (N=10/group) received anti-HBV ribozymes (100 mg/kg/day) as a continuous SC infusion. After 14 days, animals treated with a ribozyme targeting site 273 (RPI.18341) of the HBV RNA showed a significant reduction in serum HBV DNA concentration, compared to the saline treated animals as measured by a quantitative PCR assay. More specifically, the saline treated animals had a 69% increase in serum HBV DNA concentrations over this 2-week period while treatment with the 273 ribozyme (RPI.18341) resulted in a 60% decrease in serum HBV DNA concentrations. Ribozymes directed against sites 1833 (RPI.18371), 1873 (RPI.18418), and 1874 (RPI.18372) decreased serum HBV DNA concentrations by 49%, 15% and 16%, respectively.

### Example 9

### HBV Transgenic Mouse Study B

A transgenic mouse strain (founder strain 1.3.32 with a C57B1/6 background) that expresses HBV RNA and forms HBV viremia (Morrey et al., 1999, Antiviral Res., 42, 97-108; Guidotti et al., 1995, J. Virology, 69, 10, 6158-6169) was utilized to study the in vivo activity of ribozymes (RPI.18341 and RPI.18371) of the instant invention. This model is predictive in screening for anti-HBV agents. Ribozyme or the equivalent volume of saline was administered via a continuous s.c. infusion using Alzet® mini-osmotic pumps for 14 days. Alzet® pumps were filled with test material(s) in a sterile fashion according to the manufacturer's instructions. Prior to in vivo implantation, pumps were incubated at 37° C. overnight (≧18 hours) to prime the flow modulators. On the day of surgery, animals were lightly anesthetized with a ketamine/xylazine cocktail (94 mg/kg and 6 mg/kg, respectively; 0.3 ml, IP). Baseline blood samples (200 μl) were obtained from each animal via a retro-orbital bleed. For animals in groups 1-10 (Table XIII), a 2 cm area near the base of the tail was shaved and cleansed with betadine surgical scrub and sequentially with 70% alcohol. A 1 cm incision in the skin was made with a #15 scalpel blade or a blunt pair of scissors near the base of the tail. Forceps were used to open a pocket rostrally (ie., towards the head) by spreading apart the subcutaneous connective tissue. The pump was inserted with the delivery portal pointing away from the incision. Wounds were closed with sterile 9-mm stainless steel clips or with sterile 4-0 suture. Animals were then allowed to recover from anesthesia on a warm heating pad before being returned to their cage. Wounds were checked daily. Clips or sutures were replaced as needed. Incisions typically healed completely within 7 days post-op. Animals were then deeply anesthetized with the ketamine/xylazine cocktail (150 mg/kg and 10 mg/kg, respectively; 0.5 ml, IP) on day 14 post pump implantation. A midline thoracotomy/laparatomy was performed to expose the abdominal cavity and the thoracic cavity. The left ventricle was cannulated at the base and animals exsanguinated using a 23G needle and 1 ml syringe. Serum was separated, frozen and analyzed for HBV DNA and antigen levels. Experimental groups were compared to the saline control group in respect to percent change from day 0 to day 14. HBV DNA was assayed by quantitative PCR. Additionally, mice treated with 3TC® by oral gavage at a dose of 300 mg/kg/day for 14 days (group 11, Table XIII) were used as a positive control.

Results

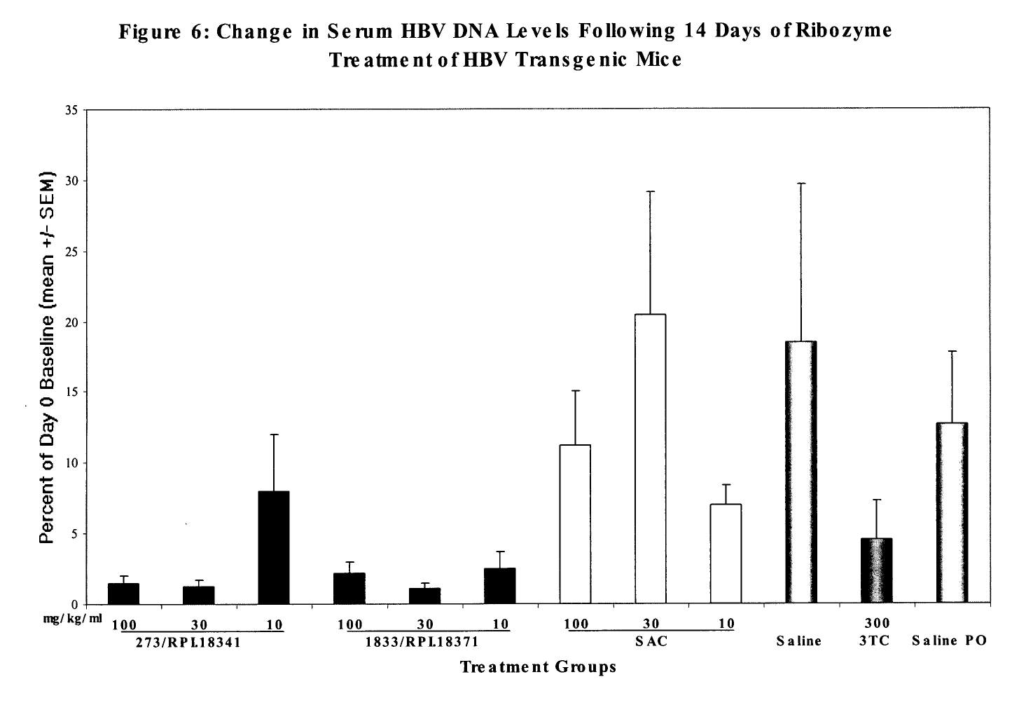

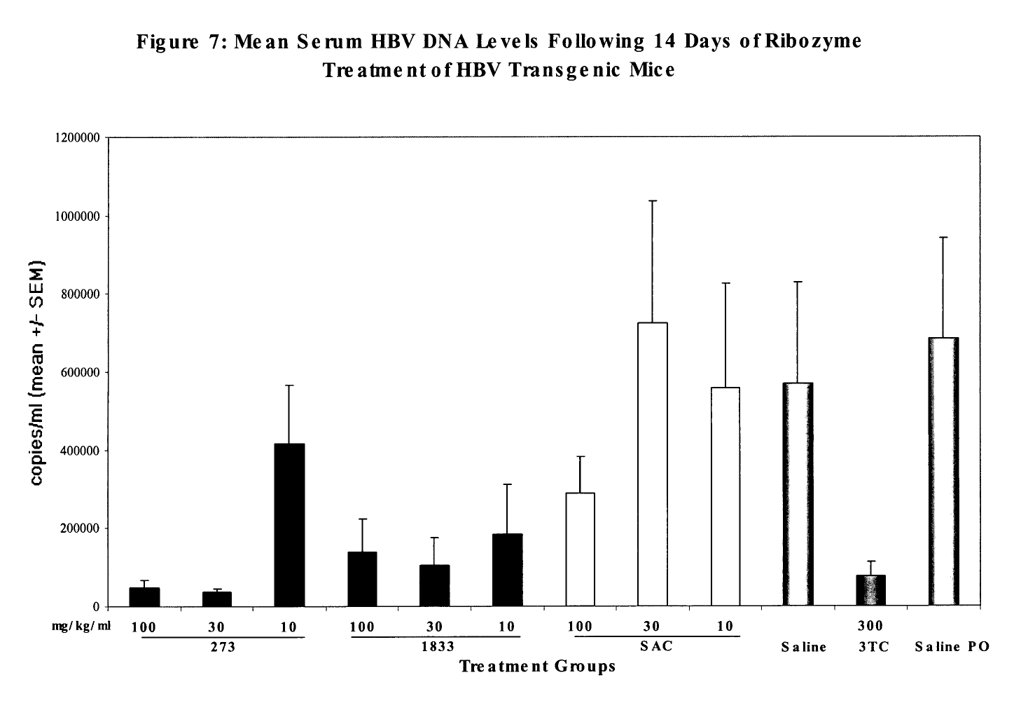

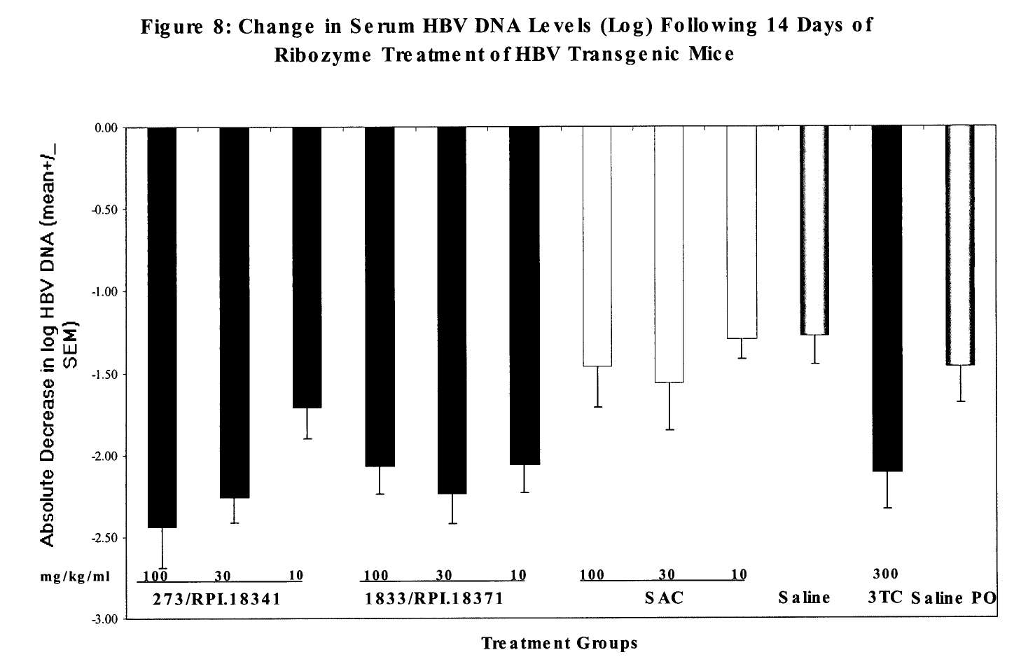

Table XIII is a summary of the group designation and dosage levels used in this HBV transgenic mouse study. Baseline blood samples were obtained via a retroorbital bleed and animals (N=15/group) received anti-HBV ribozymes (100 mg/kg/day, 30 mg/kg/day, 10 mg/kg/day) as a continuous SC infusion. The results of this study are summarized in FIGS. 6, 7, and 8. As FIGS. 6, 7, and 8 demonstrate, Ribozymes directed against sites 273 (RPI.18341) and 1833 (RPI.18371) demonstrate reduction in the serum HBV DNA levels following 14 days of ribozyme treatment in HBV transgenic mice, as compared to scrambled attenuated core (SAC) ribozyme and saline controls. Furthermore, these ribozymes provide similar, and in some cases, greater reduction of serum HBV DNA levels, as compared to the 3TC® positive control, at lower doses than the 3TC® positive control.

### Example 10

### HBV DNA Reduction in HepG2.2.15 Cells

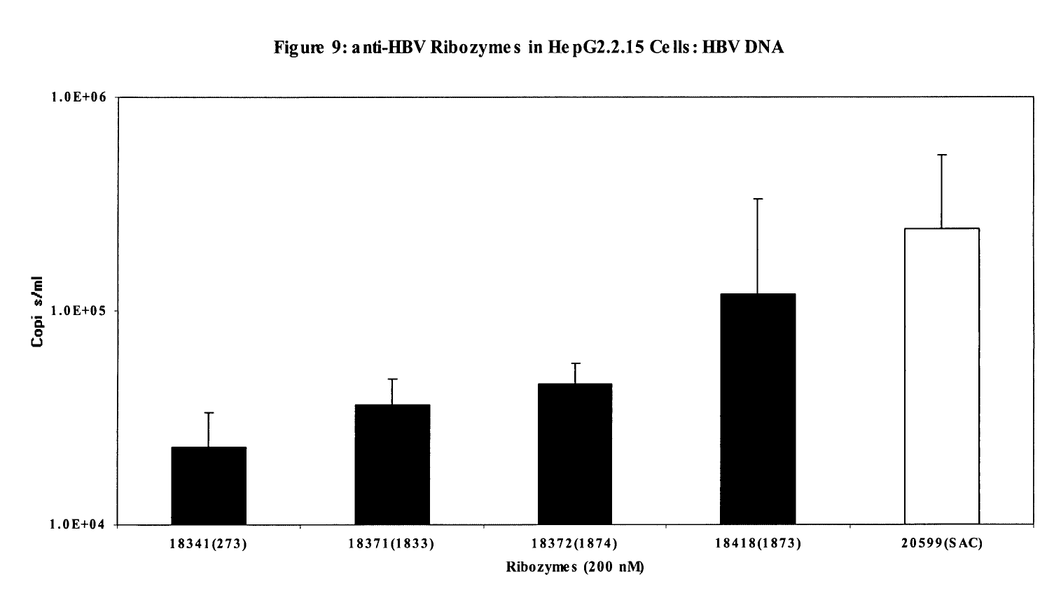

Ribozyme treatment of HepG2.2.15 cells was performed in a 96-well plate format, with 12 wells for each different ribozyme tested (RPI.18341, RPI.18371, RPI.18372, RPI.18418, RPI.20599SAC). HBV DNA levels in the media collected between 120 and 144 hours following transfection was determined using the Roche Amplicor HBV Assay. Treatment with RPI.18341 targeting site 273 resulted in a significant (P<0.05) decrease in HBV DNA levels of 62% compared to the SAC (RPI.20599). Treatment with RPI.18371 (site 1833) or RPI.18372 (site 1874) resulted in reductions in HBV DNA levels of 55% and 58% respectively, as compared to treatment with the SAC RPI.20599 (see FIG. 9).

### Example 11

### RPI 18341 Combination Treatment with Lamivudine/Infergen®

The therapeutic use of nucleic acid molecules of the invention either alone or in combination with current therapies, for example lamivudine or type 1 IFN, can lead to improved HBV treatment modalities. To assess the potential of combination therapy, HepG2 cells transfected with a replication competent HBV cDNA, were treated with RPI 18341 (HepBzyme™), Infergen® (Amgen, Thousand Oaks Calif.), and/or Lamivudine (Epivir®: GlaxoSmithKline, Research Triangle Park N.C.) either alone or in combination. Results indicated that combination treatment with either RPI 18341 plus Infergen® or combination of RPI 18341 plus lamivudine results in additive down regulation of HBsAg expression (P<0.001). These studies can be applied to the treatment of lamivudine resistant cells to further assses the potential for combination therapy of RPI 18341 plus currently available therapies for the treatment of chronic Hepatitis B.

Hep G2 cells were plated (2×104 cells/well) in 96-well microtiter plates and incubated overnight. A cationic lipid/DNA/ribozyme complex was formed containing (at final concentrations) lipid (11-15 μg/mL), re-ligated psHBV-1 (4.5 μg/mL) and ribozyme (100-200 nM) in growth media. Following a 15 min incubation at 37° C., 20 μL of the complex was added to the plated Hep G2 cells in 80 μL of growth media minus antibiotics. For combination treatment with interferon, interferon (Infergen®, Amgen, Thousand Oaks Calif.) was added at 24 hr post-transfection and then incubated for an additional 96 hr. In the case of co-treatment with Lamivudine (3TC®), the ribozyme-containing cell culture media was removed at 120 hr post-transfection, fresh media containing Lamivudine (Epivir®: GlaxoSmithKline, Research Triangle Park N.C.) was added, and then incubated for an additional 48 hours. Treatment with Lamivudine or interferon individually was done on Hep G2 cells transfected with the pSHBV-1 vector alone and then treated identically to the co-treated cells. All transfections were performed in triplicate. Analysis of HBsAg levels was performed using the Diasorin HBsAg ELISA kit.

Results

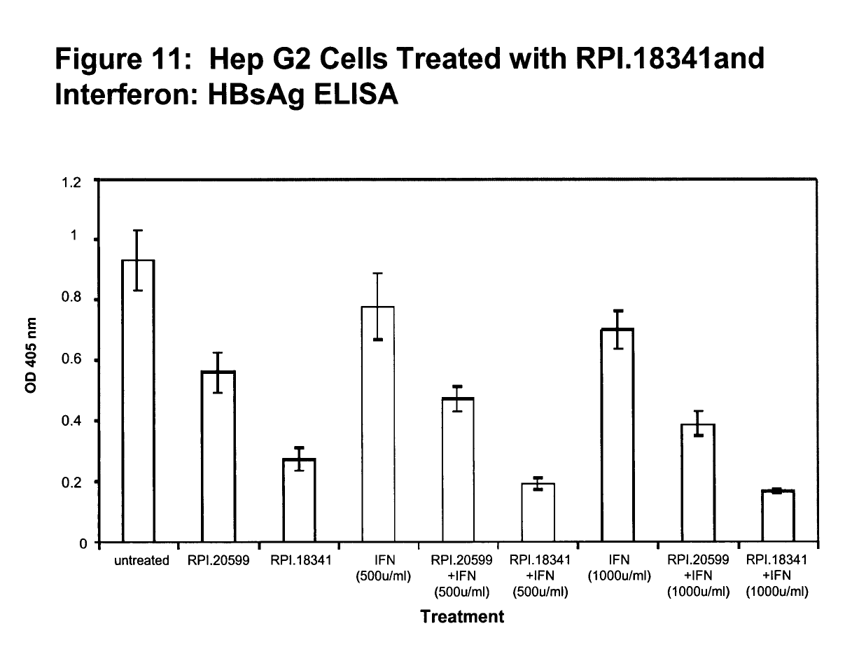

At either 500 or 1000 units of Infergen®, the addition of 200 nM of RPI.18341 results in a 75-77% increase in anti-HBV activity as judged by the level of HBsAg secreted from the treated Hep G2 cells. Conversely, the anti-HBV activity of RPI.18341(at 200 nM) is increased 31-39% when used in combination of 500 or 1000 units of Infergen® (FIG. 11).

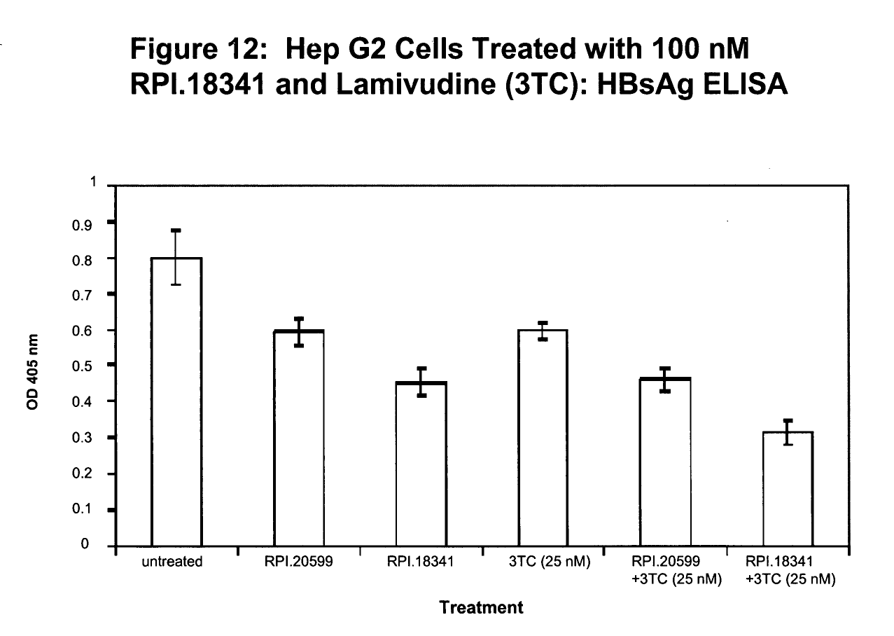

At 25 nM Lamivudine (3TC®), the addition of 100 nM of RPI.18341 results in a 48% increase in anti-HBV activity as judged by the level of HBsAg secreted from treated Hep G2 cells. Conversely, the anti-HBV activity of RPI.18341 (at 100 nM) is increased 31% when used in combination with 25 nM Lamivudine (FIG. 12).

### Example 13

### Modulation of HBV Reverse Transcriptase

The HBV reverse transcriptase (pol) binds to the 5′ stem-loop structure in the HBV pregenomic RNA and synthesizes a four-nucleotide primer from the template UUCA. The reverse transcriptase then translocates to the 3′ end of the pregenomic RNA where the primer binds to the UUCA sequence within the DR1 element and begins first-strand synthesis of HBV DNA. A number of short oligos, ranging in size from 4 to 16-mers, were designed to act as competitive inhibitors of the HBV reverse transcriptase primer, either by blocking the primer binding sites on the HBV RNA or by acting as a decoy.

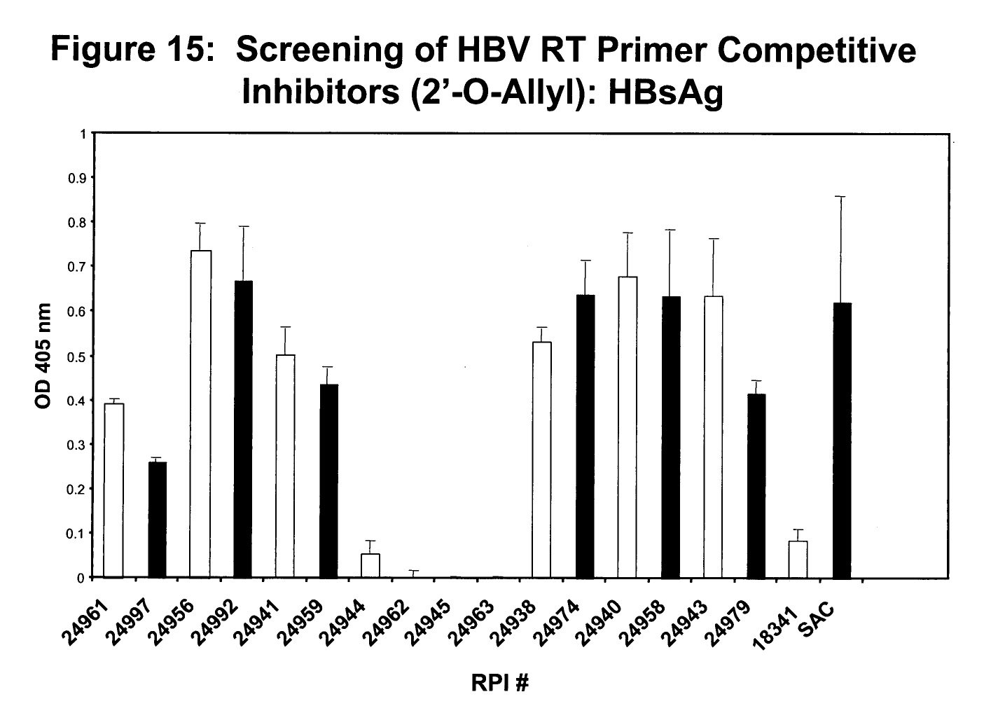

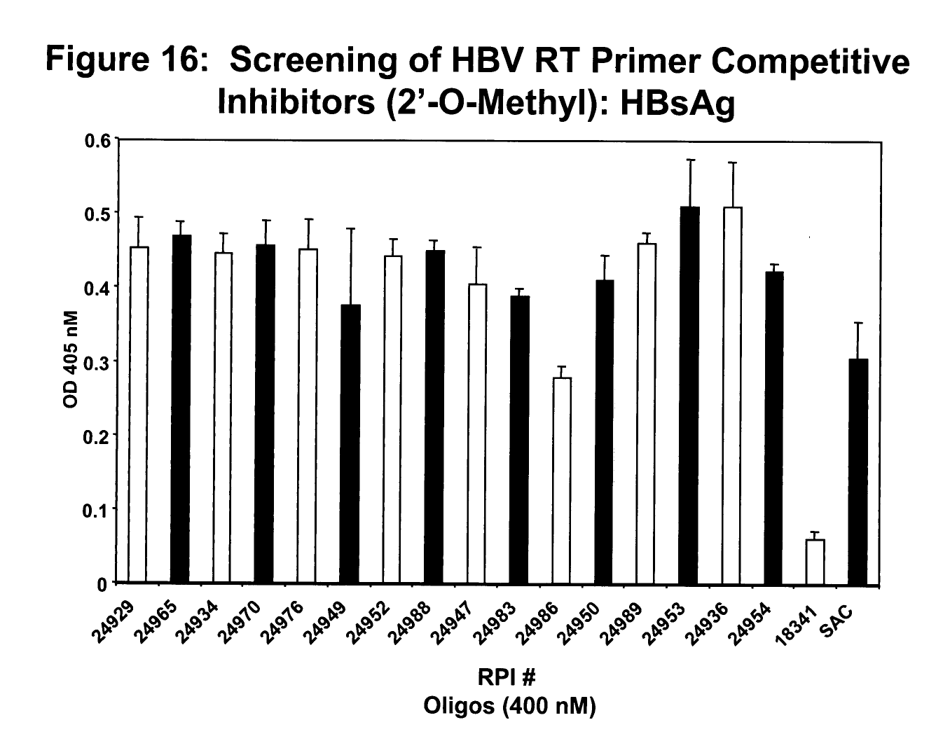

The oligonucleotides and controls were synthesized in all 2′-O-methyl and 2′-O-allyl versions (Table XV). The inverse sequence of all oligos were generated to serve as controls. Primary screening of the competitive inhibitors was completed in the HBsAg transfection/ELISA system, in which the oligo is co-transfeceted with a HBV cDNA vector into Hep G2 cells. Following 4 days of incubation, the levels of HBsAg secreted into the cell culture media were determined by ELISA. Screening of the 2′-O-allyl versions revealed that two of the decoy oligos (RPI.24944 and RPI.24945), consisting of 3× or 4× repeats of the RT primer binding site UUCA, along with the matched inverse controls, displayed considerable activity by decreasing HBsAg levels (FIG. 15). This dramatic decrease in HBsAg levels is not due to cellular toxicity, because a MTS assay showed no difference in proliferation between any of the treated cells. A follow up experiment with a 5× UUCA repeat, the inverse sequence control, and a matched scrambled control, showed that all three oligos decreased HBsAg levels without cellular toxicity. Screening of the 2′-O-methyl versions of the oligos showed no activity from the 3× and 4× UUCA repeat (FIG. 16), also suggesting that the anti-HBV effect is perhaps related to the 2′-O-allyl chemistry rather than to sequence specificity.

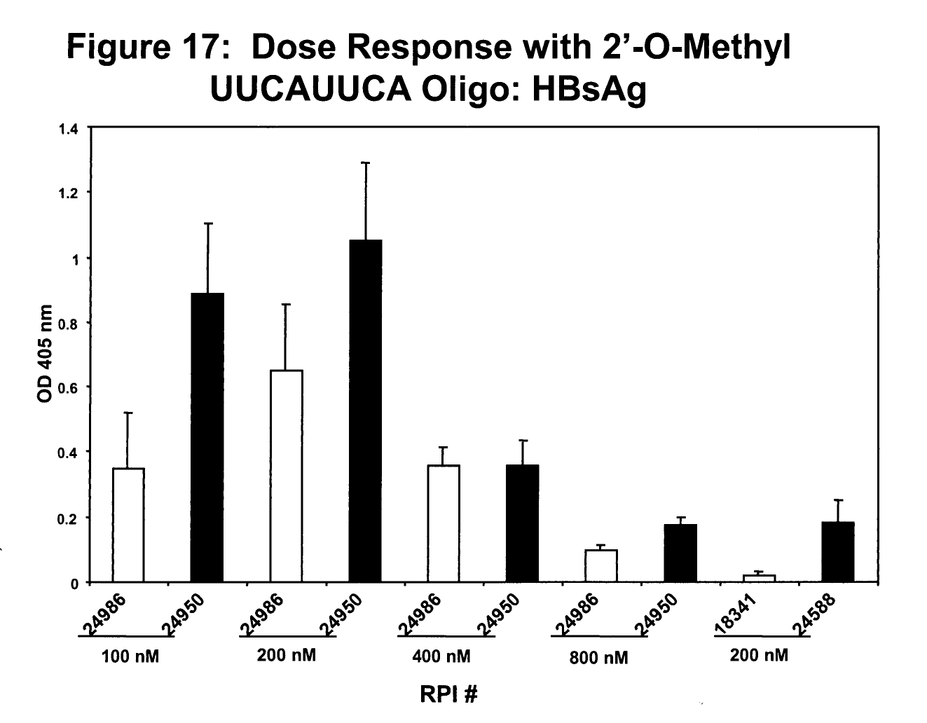

Screening of the 2′-O-methyl oligos did show that the 2′-O-methyl 2× UUCA repeat, RPI.24986, displayed activity in decreasing HBsAg levels as compared to the inverse control, RPI.24950. A dose response experiment showed that at the lower concentrations of 100 and 200 nM, RPI.24986 showed greater activity in decreasing HbsAg levels as compared to the inverse control RPI.24950 (FIG. 17).

### Example 14

### Modulation of HBV Transcription via Oligonucleotides Targeting the Enchancer I Core Region of HBV DNA

In an effort to block HBV replication, oligonucleotides were designed to bind to two liver-specific factor binding sites in the Enhancer I core region of HBV genomic DNA. Hepatocyte Nuclear Factor 3 (HNF3) and Hepatocyte Nuclear Factor 4 (HNF4) bind to sites in the core region, with the HNF3 site being 5′ to the HNF4 site. The HNF3 and HNF4 sites overlap or are adjacent to binding sites for a number of more ubiquitous factors, and are termed nuclear receptor response elements (NRRE). These elements are critical in regulating HBV transcription and replication in infected hepatocytes, with mutations in the HNF3 and HNF4 binding sites having been demonstrated to greatly reduce the levels of HBV replication (Bock et al., 2000, J. Virology, 74, 2193)

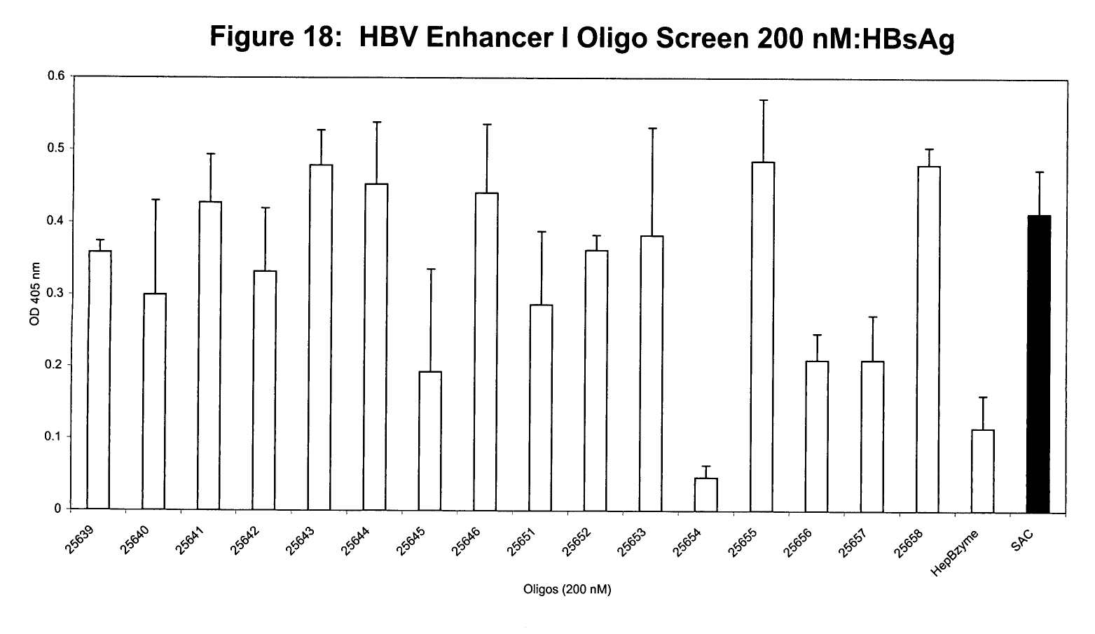

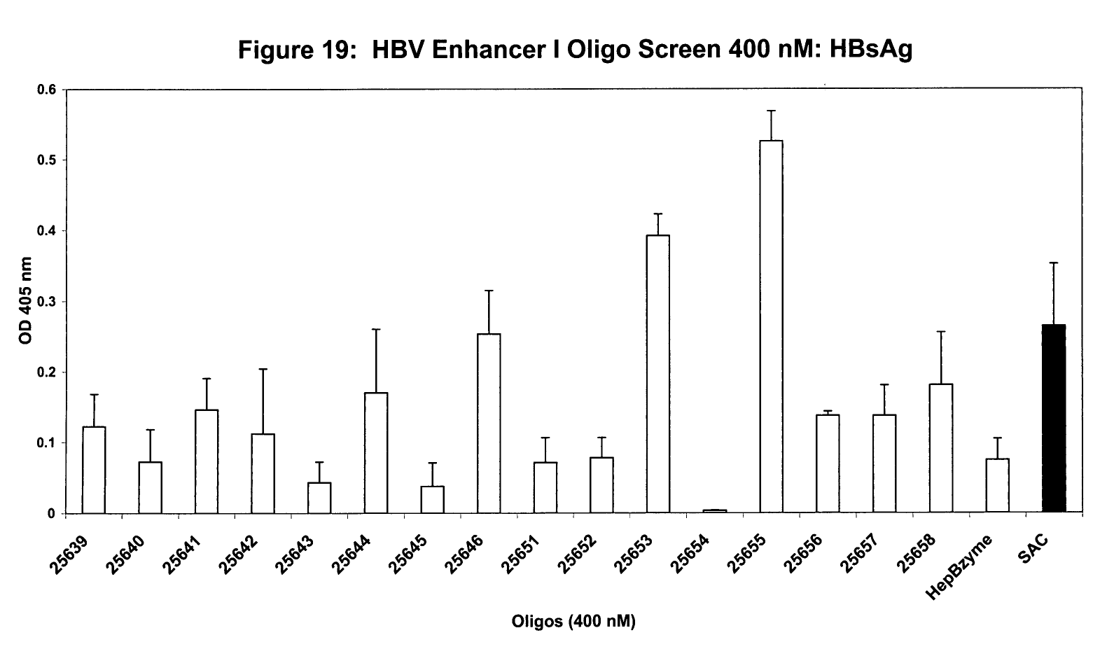

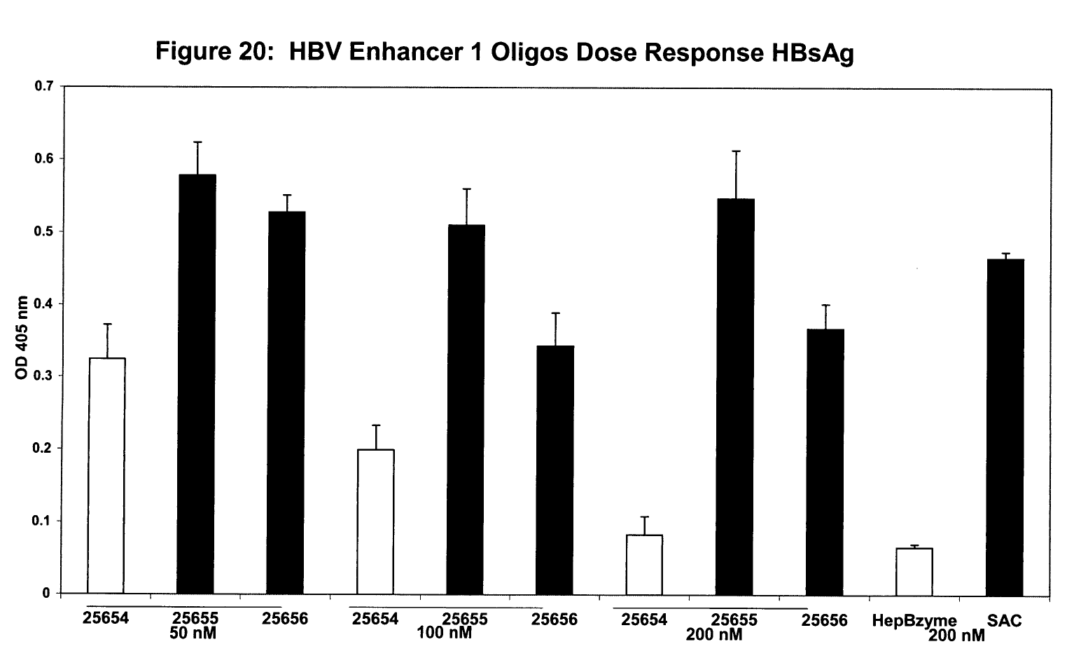

Oligonucleotides (Table XV) were designed to bind to either the positive or negative strands of the HNF3 or HNF4 binding sites. Scrambled controls were made to match each oligo. Each oligo was synthesized in all 2′-O-methyl/all phosphorothioate, or all 2′-O-allyl/all phosphorothioate chemistries. The initial screening of the oligos was done in the HBsAg transfection/ELISA system in Hep G2 cells. RPI.25654, which targets the negative strand of the HNF4 binding site, shows greater activity in reducing HBsAg levels as compared to RPI.25655, which targets the HNF4 site positive strand, and the scrambled control RPI.25656. This result was observed at both 200 and 400 nM (FIGS. 18 and 19). In a follow-up study, RPI.25654 reduced HBsAg levels in a dose-dependent manner, from 50-200 nM (FIG. 20).

### Example 15

### Transfection of HepG2 Cells with psHBV-1 and Nucleic Acid

The human hepatocellular carcinoma cell line Hep G2 was grown in Dulbecco's modified Eagle media supplemented with 10% fetal calf serum, 2 mM glutamine, 0.1 mM nonessential amino acids, 1 mM sodium pyruvate, 25 mM Hepes, 100 units penicillin, and 100 μg/ml streptomycin. To generate a replication competent cDNA, prior to transfection the HBV genomic sequences are excised from the bacterial plasmid sequence contained in the psHBV-1 vector This was done with an EcoRI and Hind III restriction digest. Following completion of the digest, a ligation was performed under dilute conditions (20 μg/ml) to favor intermolecular ligation. The total ligation mixture was then concentrated using Qiagen spin columns. One skilled in the art would realize that other methods can be used to generate a replication competent cDNA Secreted alkaline phosphatase (SEAP) was used to normalize the HBsAg levels to control for transfection variability. The pSEAP2-TK control vector was constructed by ligating a Bgl II-Hind III fragment of the pRL-TK vector (Promega), containing the herpes simplex virus thymidine kinase promoter region, into Bgl II/Hind III digested pSEAP2-Basic (Clontech). Hep G2 cells were plated (3×10⁴ cells/well) in 96-well microtiter plates and incubated overnight. A lipid/DNA/nucleic acid complex was formed containing (at final concentrations) cationic lipid (15 μg/ml), prepared psHBV-1 (4.5 μg/ml), pSEAP2-TK (0.5 μg/ml), and nucleic acid (100 μM). Following a 15 min. incubation at 37° C., the complexes were added to the plated Hep G2 cells. Media was removed from the cells 96 hr. post-transfection for HBsAg and SEAP analysis.

Transfection of the human hepatocellular carcinoma cell line, Hep G2, with replication competent HBV DNA results in the expression of HBV proteins and the production of virions.

### Example 16

### Analysis of HBsAg and SEAP Levels Following Nucleic Acid Treatment

Immulon 4 (Dynax) microtiter wells were coated overnight at 4° C. with anti-HBsAg Mab (Biostride B88-95-31ad,ay) at 1 μg/ml in Carbonate Buffer (Na2CO3 15 mM, NaHCO3 35 mM, pH 9.5). The wells were then washed 4× with PBST (PBS, 0.05% Tween® 20) and blocked for 1 hr at 37° C. with PBST, 1% BSA. Following washing as above, the wells were dried at 37° C. for 30 min. Biotinylated goat anti-HBsAg (Accurate YVS1807) was diluted 1:1000 in PBST and incubated in the wells for 1 hr. at 37° C. The wells were washed 4× with PBST. Streptavidin/Alkaline Phosphatase Conjugate (Pierce 21324) was diluted to 250 ng/ml in PBST, and incubated in the wells for 1 hr. at 37° C. After washing as above, p-nitrophenyl phosphate substrate (Pierce 37620) was added to the wells, which were then incubated for 1 hr. at 37° C. The optical density at 405 nm was then determined. SEAP levels were assayed using the Great EscAPe® Detection Kit (Clontech K2041-1), as per the manufacturers instructions.

### Example 17

### Analysis of HBV DNA Expression a HepG2.2.15 Murine Model

The development of new antiviral agents for the treatment of chronic Hepatitis B has been aided by the use of animal models that are permissive to replication of related Hepadnaviridae such as Woodchuck Hepatitis Virus (WHV) and Duck Hepatitis Virus (DHV). In addition, the use of transgenic mice has also been employed. The human hepatoblastoma cell line, HepG2.2.15, implanted as a subcutaneous (SC) tumor, can be used to produce Hepatitis B viremia in mice. This model is useful for evaluating new HBV therapies. Mice bearing HepG2.2.15 SC tumors show HBV viremia. HBV DNA can be detected in serum beginning on Day 35. Maximum serum viral levels reach 1.9×10⁵ copies/mL by day 49. A study also determined that the minimum tumor volume associated with viremia was 300 mm³. Therefore, the HepG2.2.15 cell line grown as a SC tumor produces a useful model of HBV viremia in mice. This new model can be suitable for evaluating new therapeutic regimens for chronic Hepatitis B.

HepG2.2.15 tumor cells contain a slightly truncated version of viral HBV DNA and sheds HBV particles. The purpose of this study was to identify what time period viral particles are shed from the tumor. Serum was analyzed for presence of HBV DNA over a time course after HepG2.2.15 tumor inoculation in Athymic Ncr nu/nu mice. HepG2.2.15 cells were carried and expanded in DMEM/10% FBS/2.4% HEPES/1% NEAA/1% Glutamine/1% Sodium Pyruvate media. Cells were resuspended in Delbecco's PBS with calcium/magnesium for injection. One hundred microliters of the tumor cell suspension (at a concentration of 1×108 cells/mL) were injected subcutaneously in the flank of NCR nu/nu female mice with a 23 gl needle and 1 cc syringe, thereby giving each mouse 1×10⁷ cells. Tumors were allowed to grow for a period of up to 49 days post tumor cell inoculation. Serum was sampled for analysis on days 1, 7, 14, 35, 42 and 49 post tumor inoculation. Length and width measurements from each tumor were obtained three times per week using a Jamison microcaliper. Tumor volumes were calculated from tumor length/width measurements (tumor volume=0.5[a(b)²] where a=longest axis of the tumor and b=shortest axis of the tumor). Serum was analyzed for the presence of HBV DNA by the Roche Amplicor HBV moniter TM DNA assay.

Experiment 1

HepG2.2.15 cells were carried and expanded in DMEM/10% FBS/2.4% HEPES/1% NEAA/1% Glutamine/1% Sodium Pyruvate media. Cells were resuspended in Delbecco's PBS with calcium/magnesium for injection. One hundred microliters of the tumor cell suspension (at a concentration of 1×108 cells/mL) were injected subcutaneously in the flank of NCR nu/nu female mice with a 23 gl needle and 1 cc syringe, thereby giving each mouse 1×10⁷ cells. Tumors were allowed to grow for a period of up to 49 days post tumor cell inoculation. Serum was sampled for analysis on days 1, 7, 14, 35, 42 and 49 post tumor inoculation. Length and width measurements from each tumor were obtained three times per week using a Jamison microcaliper. Tumor volumes were calculated from tumor length/width measurements (tumor volume=0.5[a(b)²] where a=longest axis of the tumor and b=shortest axis of the tumor). Serum was analyzed for the presence of HBV DNA by the Roche Amplicor HBV moniter TM DNA assay.

Results

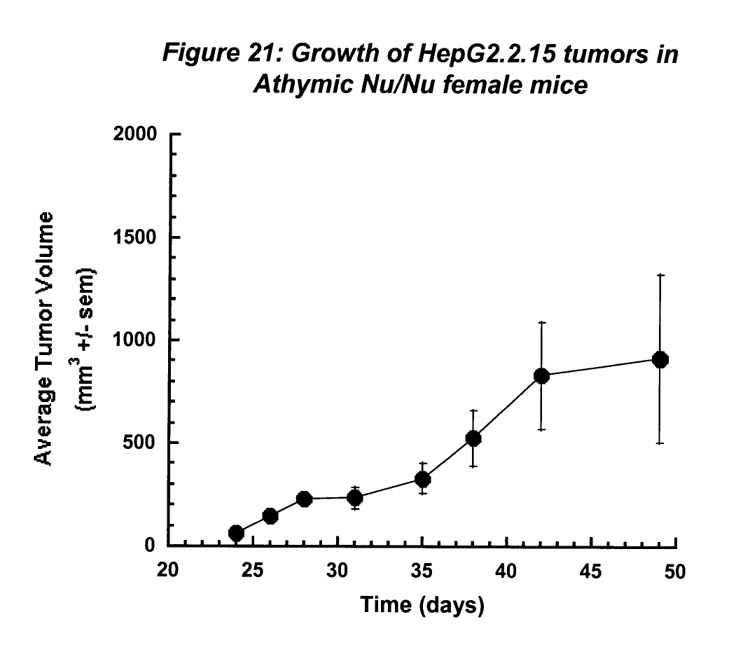

When athymic nu/nu female mice are subcutaneously injected with HepG2.2.15 cells and form tumors, HBV DNA is detected in serum (peak serum level was 1.9×10⁵ copies/mL). There is a positive correlation (rs=0.7, p<0.01) between tumor weight (milligrams) and HB viral copies/mL serum. FIG. 21 shows a plot of HepG2.2.15 tumors in nu/nu female mice as tumor volume vs time. Table XVI shows the concentration of HBV DNA in relation to tumor size in the HepG2.2.15 implanted nu/nu female mice used in the study.

Experiment 2

HepG2.2.15 cells were carried and expanded in DMEM/10% FBS/2.4% HEPES/1% NEAA/1% Glutamine/1% Sodium Pyruvate media containing 400 μg/ml G418 antibiotic. G418-resistant cells were resuspended in Dulbecco's PBS with calcium/magnesium for injection. One hundred microliters of the tumor cell suspension (at a concentration of 1×108 cells/mL) were injected subcutaneously in the flank of NCR nu/nu female mice with a 23 gl needle and 1 cc syringe, thereby giving each mouse 1×10⁷ cells. Tumors were allowed to grow for a period of up to 49 days post tumor cell inoculation. Serum was sampled for analysis on day 37 post tumor inoculation. Length and width measurements from each tumor were obtained three times per week using a Jamison microcaliper. Tumor volumes were calculated from tumor length/width measurements (tumor volume=0.5[a(b)²] where a=longest axis of the tumor and b=shortest axis of the tumor). Serum was analyzed for the presence of HBV DNA by the Roche Amplicor HBV moniter TM DNA assay.

Results

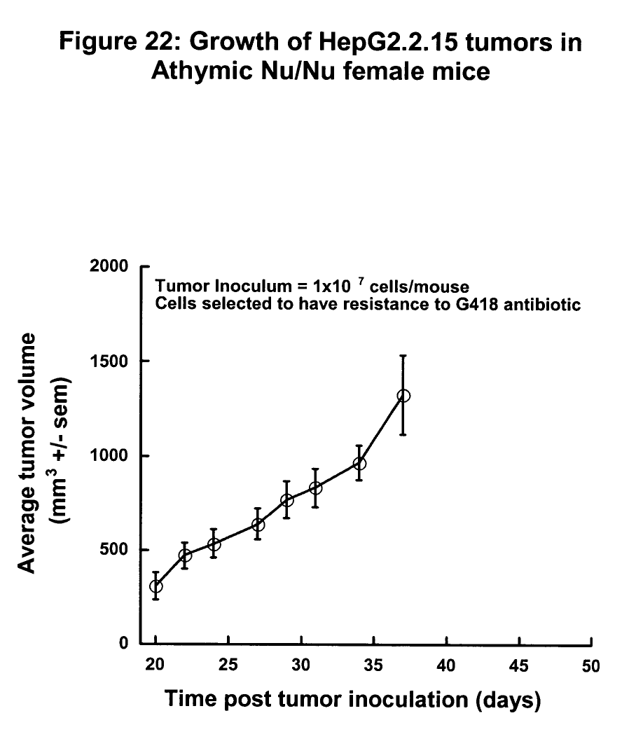

When athymic nu/nu female mice are subcutaneously injected with G418 antibiotic resistant HepG2.2.15 cells and form tumors, HBV DNA is detected in serum (peak serum level was 4.0×10⁵ copies/mL). There is a positive correlation (rs=0.7, p<0.01) between tumor weight (milligrams) and HB viral copies/mL serum. FIG. 22 shows a plot of HepG2.2.15 tumors in nu/nu female mice as tumor volume vs time. Table XVII shows the concentration of HBV DNA in relation to tumor size in the G418 antibiotic resistant HepG2.2.15 implanted nu/nu female mice used in the study.

### Example 18

### Identification of Potential Enzymatic Nucleic Acid Molecules Cleavage Sites in HCV RNA

The sequence of HCV RNA was screened for accessible sites using a computer folding algorithm. Regions of the mRNA that did not form secondary folding structures and contained potential enzymatic nucleic acid cleavage sites were identified. The sequences of these cleavage sites are shown in Tables XVIII, XIX, XX and XXIII.

### Example 19

### Selection of Enzymatic Nucleic Acid Molecules Cleavage Sites in HCV RNA

Enzymatic nucleic acid target sites were chosen by analyzing sequences of Human HCV (Genbank accession Nos: D11168, D50483.1, L38318 and S82227) and prioritizing the sites on the basis of folding. Enzymatic nucleic acid molecules are designed that could bind each target and are individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the enzymatic nucleic acid molecules sequences fold into the appropriate secondary structure. Those enzymatic nucleic acid molecules with unfavorable intramolecular interactions between the binding arms and the catalytic core can be eliminated from consideration. As noted below, varying binding arm lengths can be chosen to optimize activity. Generally, at least 4 bases on each arm are able to bind to, or otherwise interact with, the target RNA.

### Example 20

### Chemical Synthesis and Purification of Enzymatic Nucleic Acids

Enzymatic nucleic acid molecules can be designed to anneal to various sites in the RNA message. The binding arms of the enzymatic nucleic acid molecules are complementary to the target site sequences described above. The enzymatic nucleic acid molecules can be chemically synthesized using, for example, RNA syntheses such as those described above and those described in Usman et al., (1987 J. Am. Chem. Soc., 109, 7845), Scaringe et al., (1990 Nucleic Acids Res., 18, 5433) and Wincott et al., supra. Such methods make use of common nucleic acid protecting and coupling groups, such as dimethoxytrityl at the 5′-end, and phosphoramidites at the 3′-end. The average stepwise coupling yields are typically >98%. Enzymatic nucleic acid molecules can be modified to enhance stability by modification with nuclease resistant groups, for example, 2′-amino, 2′-C-allyl, 2′-flouro, 2′-O-methyl, 2′-H (for a review see Usman and Cedergren, 1992 TIBS 17, 34).

Enzymatic nucleic acid molecules can also be synthesized from DNA templates using bacteriophage T7 RNA polymerase (Milligan and Uhlenbeck, 1989, Methods Enzymol. 180, 51). Enzymatic nucleic acid molecules can be purified by gel electrophoresis using known methods, or can be purified by high pressure liquid chromatography (HPLC; See Wincott et al., supra; the totality of which is hereby incorporated herein by reference), and are resuspended in water. The sequences of chemically synthesized enzymatic nucleic acid constructs are shown below in Tables XX, XXI and XXIII. The antisense nucleic acid molecules shown in Table XXII were chemically synthesized.

Inactive enzymatic nucleic acid molecules, for example inactive hammerhead enzymatic nucleic acids, can be synthesized by substituting the order of G5A6 and substituting a U for A14 (numbering from Hertel et al., 1992 Nucleic Acids Res., 20, 3252).

### Example 21

### Enzymatic Nucleic Acid Cleavage of HCV RNA Target In Vitro

Enzymatic nucleic acid molecules targeted to the HCV are designed and synthesized as described above. These enzymatic nucleic acid molecules can be tested for cleavage activity in vitro, for example using the following procedure. The target sequences and the nucleotide location within the HCV are given in Tables XVIII, XIX, XX and XXIII.

Cleavage Reactions: Full-length or partially full-length, internally-labeled target RNA for enzymatic nucleic acid molecule cleavage assay is prepared by in vitro transcription in the presence of [α-³²P] CTP, passed over a G 50 Sephadex column by spin chromatography and used as substrate RNA without further purification. Alternately, substrates are 5′-³²P-end labeled using T4 polynucleotide kinase enzyme. Assays are performed by pre-warming a 2× concentration of purified enzymatic nucleic acid molecule in enzymatic nucleic acid molecule cleavage buffer (50 mM Tris-HCl, pH 7.5 at 37° C., 10 mM MgCl₂) and the cleavage reaction was initiated by adding the 2× enzymatic nucleic acid molecule mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM enzymatic nucleic acid molecule, i.e., enzymatic nucleic acid molecule excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by enzymatic nucleic acid molecule cleavage are visualized on an autoradiograph of the gel. The percentage of cleavage is determined by Phosphor Imager® quantitation of bands representing the intact substrate and the cleavage products.

Alternatively, enzymatic nucleic acid molecules and substrates were synthesized in 96-well format using 0.2 μmol scale. Substrates were 5′-³²P labeled and gel purified using 7.5% polyacrylamide gels, and eluting into water. Assays were done by combining trace substrate with 500 nM enzymatic nucleic acid or greater, and initiated by adding final concentrations of 40 mM Mg⁺², and 50 mM Tris-Cl pH 8.0. For each enzymatic nucleic acid/substrate combination a control reaction was done to ensure cleavage was not the result of non-specific substrate degradation. A single three hour time point was taken and run on a 15% polyacrylamide gel to asses cleavage activity. Gels were dried and scanned using a Molecular Dynamics Phosphorimager and quantified using Molecular Dynamics ImageQuant software. Percent cleaved was determined by dividing values for cleaved substrate bands by full-length (uncleaved) values plus cleaved values and multiplying by 100 (% cleaved=[C/(U+C)]*100). In vitro cleavage data of enzymatic nucleic acid molecules targeting plus and minus strand HCV RNA is shown in Table XXIII.

### Example 22

### Inhibition of Luciferase Activity Using HCV Targeting Enzymatic Nucleic Acids in OST7 Cells

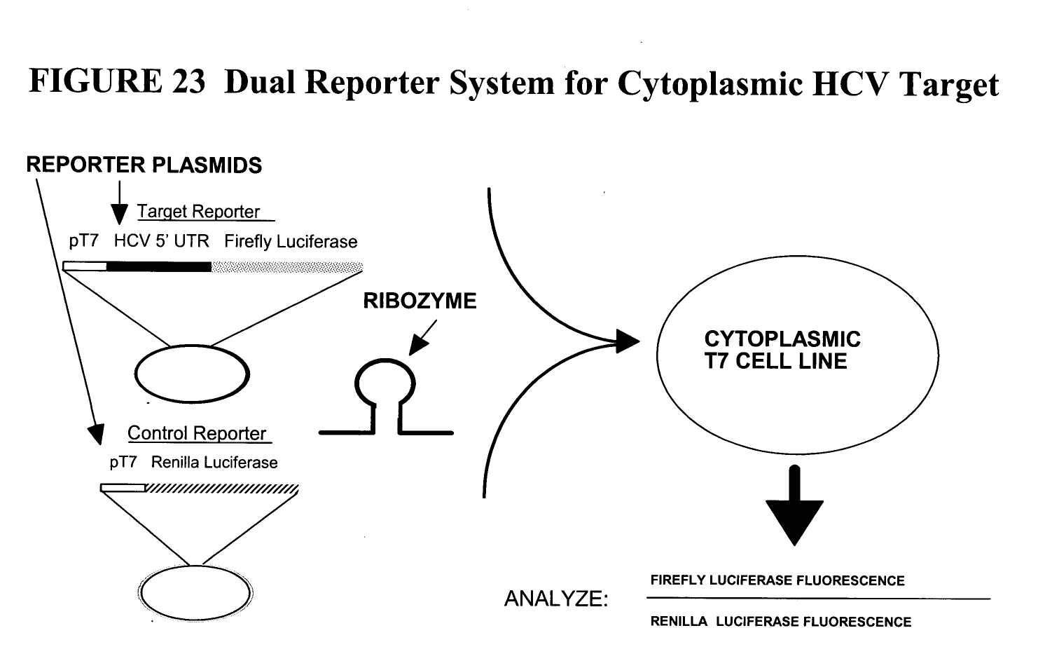

The capability of enzymatic nucleic acids to inhibit HCV RNA intracellularly was tested using a dual reporter system that utilizes both firefly and Renilla luciferase (FIG. 23). The enzymatic nucleic acids targeted to the 5′ HCV UTR region, which when cleaved, would prevent the translation of the transcript into luciferase.

Synthesis of Stabilized Enzymatic Nucleic Acids

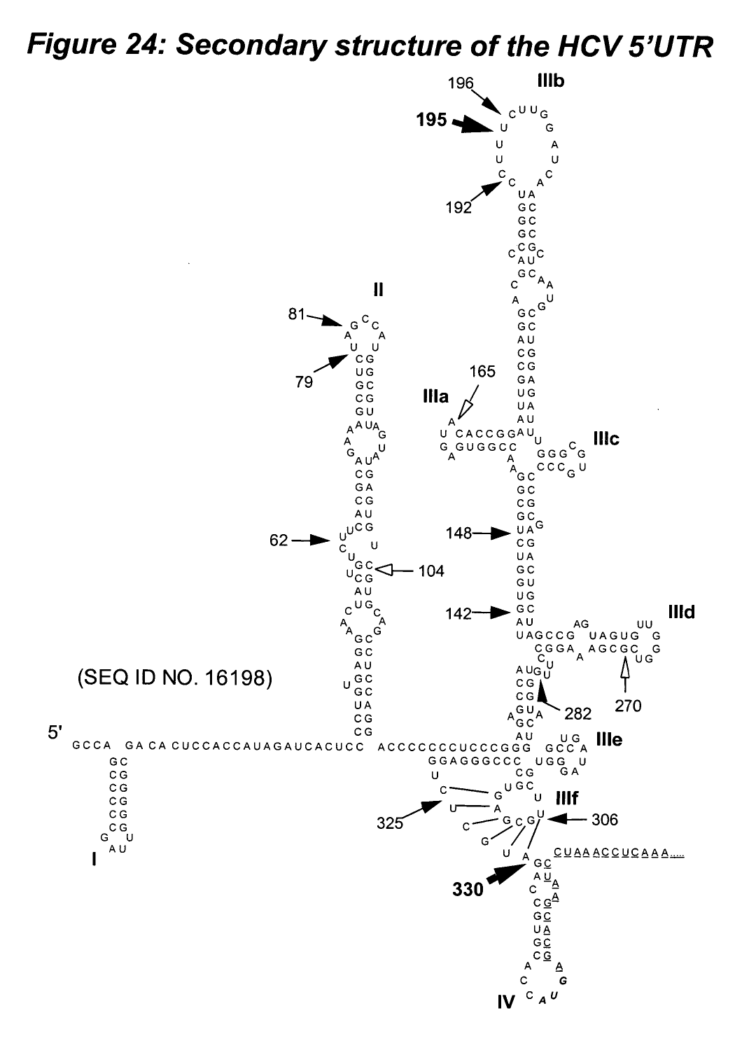

Enzymatic nucleic acids were designed to target 15 sites within the 5′UTR of the HCV RNA (FIG. 24) and synthesized as previously described, except that all enzymatic nucleic acids contain two 2′-amino uridines. Enzymatic nucleic acid and paired control sequences for targeted sites used in various examples herein are shown in Table XXI.

Reporter Plasmids

The T7/HCV/firefly luciferase plasmid (HCVT7C₁₋₃₄₁, genotype 1a) was graciously provided by Aleem Siddiqui (University of Colorado Health Sciences Center, Denver, Colo.). The T7/HCV/firefly luciferase plasmid contains a T7 bacteriophage promoter upstream of the HCV 5′UTR (nucleotides 1-341)/firefly luciferase fusion DNA. The Renilla luciferase control plasmid (pRLSV40) was purchased from PROMEGA.

Luciferase Assay

Dual luciferase assays were carried out according to the manufacturer's instructions (PROMEGA) at 4 hours after co-transfection of reporter plasmids and enzymatic nucleic acids. All data is shown as the average ratio of HCV/firefly luciferase luminescence over Renilla luciferase luminescence as determined by triplicate samples ±SD.

Cell Culture and Transfections

OST7 cells were maintained in Dulbecco's modified Eagle's medium (GIBCO BRL) supplemented with 10% fetal calf serum, L-glutamine (2 mM) and penicillin/streptomycin. For transfections, OST7 cells were seeded in black-walled 96-well plates (Packard) at a density of 12,500 cells/well and incubated at 37° C. under 5% CO₂ for 24 hours. Co-transfection of target reporter HCVT7C (0.8 μg/mL), control reporter pRLSV40, (1.2 μg/mL) and enzymatic nucleic acid, (50-200 nM) was achieved by the following method: a 5× mixture of HCVT7C (4 μg/mL), pRLSV40 (6 μg/mL) enzymatic nucleic acid (250-1000 nM) and cationic lipid (28.5 μg/mL) was made in 150 μL of OPTI-MEM (GIBCO BRL) minus serum. Reporter/enzymatic nucleic acid/lipid complexes were allowed to form for 20 min at 37° C. under 5% CO₂. Medium was aspirated from OST7 cells and replaced with 120 μL of OPTI-MEM (GIBCO BRL) minus serum, immediately followed by the addition of 30 μL of 5× reporter/enzymatic nucleic acid/lipid complexes. Cells were incubated with complexes for 4 hours at 37° C. under 5% CO₂.

IC50 Determinations for Dose Response Curves

Apparent IC₅₀ values were calculated by linear interpolation. The apparent IC₅₀ is 1/2 the maximal response between the two consecutive points in which approximately 50% inhibition of HCV/luciferase expression is observed on the dose curve.

Quantitation of RNA Samples

Total RNA from transfected cells was purified using the Qiagen RNeasy 96 procedure including a DNase I treatment according to the manufacturer's instructions. Real time RT-PCR (Taqman assay) was performed on purified RNA samples using separate primer/probe sets specific for either firefly or Renilla luciferase RNA. Firefly luciferase primers and probe were upper (5′-CGGTCGGTAAAGTTGTTCCATT-3′) (SEQ ID NO. 16202), lower (5′-CCTCTGACACATAATTCGCCTCT-3′) (SEQ ID NO. 16203), and probe (5′-FAM-TGAAGCGAAGGTTGTGGATCTGGATACC-TAMRA-3′) (SEQ ID NO 16204), and Renilla luciferase primers and probe were upper (5′-GTTTATTGAATCGGACCCAGGAT-3′) (SEQ ID NO. 16205), lower (5′-AGGTGCATCTTCTTGCGAAAA-3′) (SEQ ID NO. 16206), and probe (5′-FAM-CTTTTCCAATGCTATTGTTGAAGGTGCCAA-3′) (SEQ ID NO. 16207)-TAMRA, both sets of primers and probes were purchased from Integrated DNA Technologies. RNA levels were determined from a standard curve of amplified RNA purified from a large-scale transfection. RT minus controls established that RNA signals were generated from RNA and not residual plasmid DNA. RT-PCR conditions were: 30 min at 48° C., 10 min at 95° C., followed by 40 cycles of 15 sec at 95° C. and 1 min at 60° C. Reactions were performed on an ABI Prism 7700 sequence detector. Levels of firefly luciferase RNA were normalized to the level of Renilla luciferase RNA present in the same sample. Results are shown as the average of triplicate treatments ±SD.

### Example 23

### Inhibition of HCV 5′UTR-Luciferase Expression by Synthetic Stabilized Enzymatic Nucleic Acids

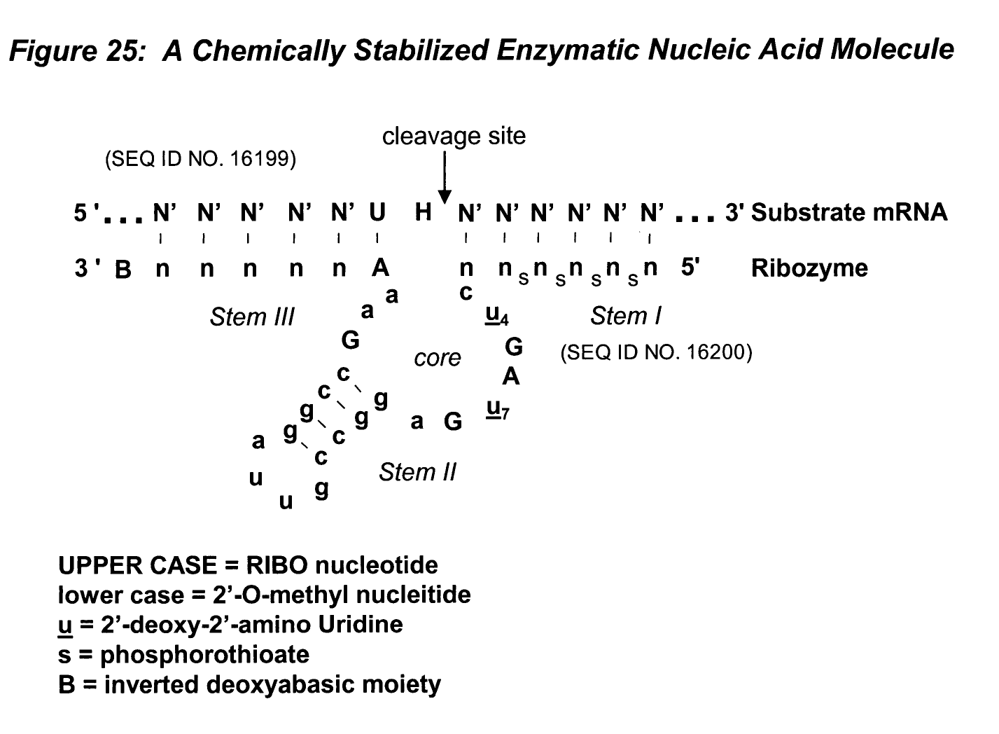

The primary sequence of the HCV 5′UTR and characteristic secondary structure (FIG. 24) is highly conserved across all HCV genotypes, thus making it a very attractive target for enzymatic nucleic acid-mediated cleavage. Enzymatic hammerhead nucleic acids, as a generally shown in FIG. 25 and Table XXI (RPI 12249-12254, 12257-12265) were designed and synthesized to target 15 of the most highly conserved sites in the 5′UTR of HCV RNA. These synthetic enzymatic nucleic acids were stabilized against nuclease degradation by the addition of modifications such as 2′-O-methyl nucleotides, 2′-amino-uridines at U4 and U7 core positions, phosphorothioate linkages, and a 3′-inverted abasic cap.

In order to mimic cytoplasmic transcription of the HCV genome, OST7 cells were transfected with a target reporter plasmid containing a T7 bacteriophage promoter upstream of a HCV 5′UTR/firefly luciferase fusion gene. Cytoplasmic expression of the target reporter is facilitated by high levels of T7 polymerase expressed in the cytoplasm of OST7 cells. Co-transfection of target reporter HCVT7C₁₋₃₄₁ (firefly luciferase), control reporter pRLSV40 (Renilla luciferase) and enzymatic nucleic acid was carried out in the presence of cationic lipid. To determine the background level of luciferase activity, applicant used a control enzymatic nucleic acid that targets an irrelevant, non-HCV sequence. Transfection of reporter plasmids in the presence of this irrelevant control enzymatic nucleic acid (ICR) resulted in a slight decrease of reporter expression when compared to transfection of reporter plasmids alone. Therefore, the ICR was used to control for non-specific effects on reporter expression during treatment with HCV specific enzymatic nucleic acids. Renilla luciferase expression from the pRLSV40 reporter was used to normalize for transfection efficiency and sample recovery.

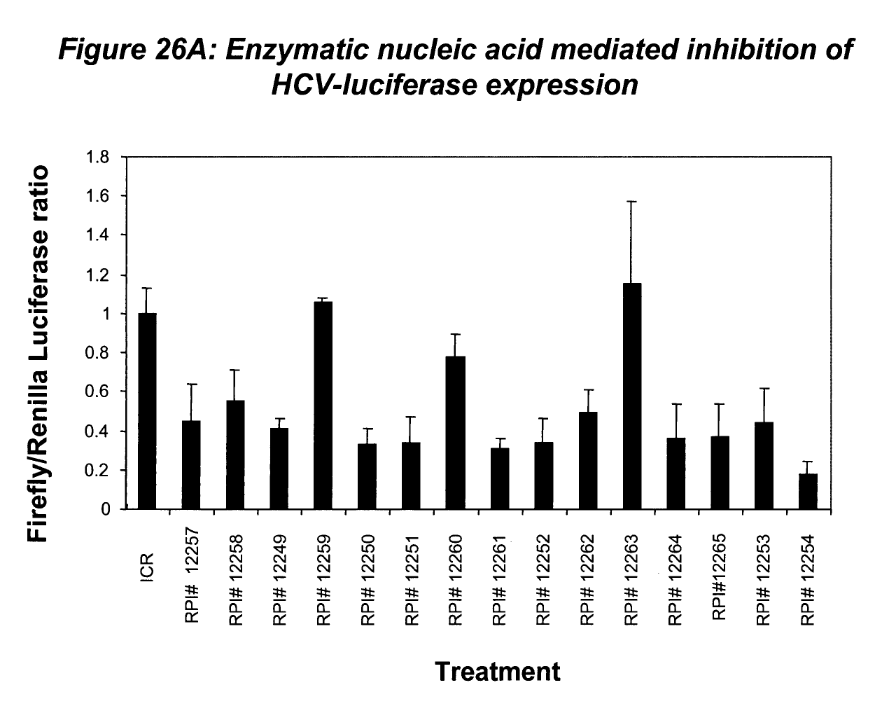

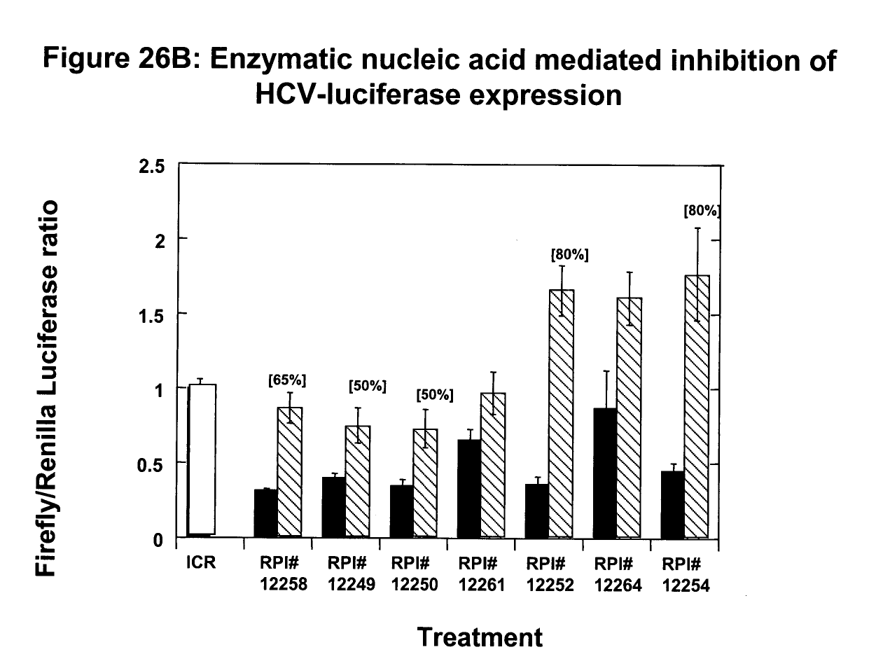

Of the 15 amino-modified hammerhead enzymatic nucleic acids tested, 12 significantly inhibited HCV/luciferase expression (>45%, P<0.05) as compared to the ICR (FIG. 26A). These data suggest that most of the HCV 5′UTR sites targeted here are accessible to enzymatic nucleic acid binding and subsequent RNA cleavage. To investigate further the enzymatic nucleic acid-dependent inhibition of HCV/luciferase activity, hammerhead enzymatic nucleic acids designed to cleave after sites 79, 81, 142, 192, 195, 282 or 330 of the HCV 5′UTR were selected for continued study because their anti-HCV activity was the most efficacious over several experiments. A corresponding attenuated core (AC) control was synthesized for each of the 7 active enzymatic nucleic acids (Table XX). Each paired AC control contains similar nucleotide composition to that of its corresponding active enzymatic nucleic acid however, due to scrambled binding arms and changes to the catalytic core, lacks the ability to bind or catalyze the cleavage of HCV RNA. Treatment of OST7 cells with enzymatic nucleic acids designed to cleave after sites 79, 81, 142, 195 or 330 resulted in significant inhibition of HCV/luciferase expression (65%, 50%, 50%, 80% and 80%, respectively) when compared to HCV/luciferase expression in cells treated with corresponding ACs, P<0.05 (FIG. 26B). It should be noted that treatment with either the ICR or ACs for sites 79, 81, 142 or 192 caused a greater reduction of HCV/luciferase expression than treatment with ACs for sites 195, 282 or 330. The observed differences in HCV/luciferase expression after treatment with ACs most likely represents the range of activity due to non-specific effects of oligonucleotide treatment and/or differences in base composition. Regardless of differences in HCV/luciferase expression levels observed as a result of treatment with ACs, active enzymatic nucleic acids designed to cleave after sites 79, 81, 142, 195, or 330 demonstrated similar and potent anti-HCV activity (FIG. 26B).

### Example 24

### Synthetic Stabilized Enzymatic Nucleic Acids Inhibit HCV/Luciferase Expression in a Concentration-Dependent Manner

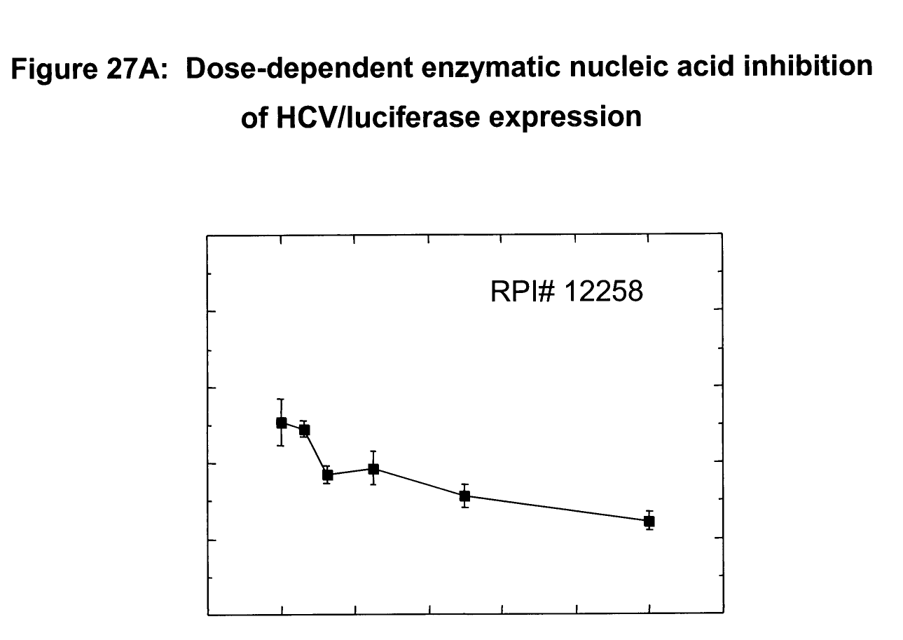

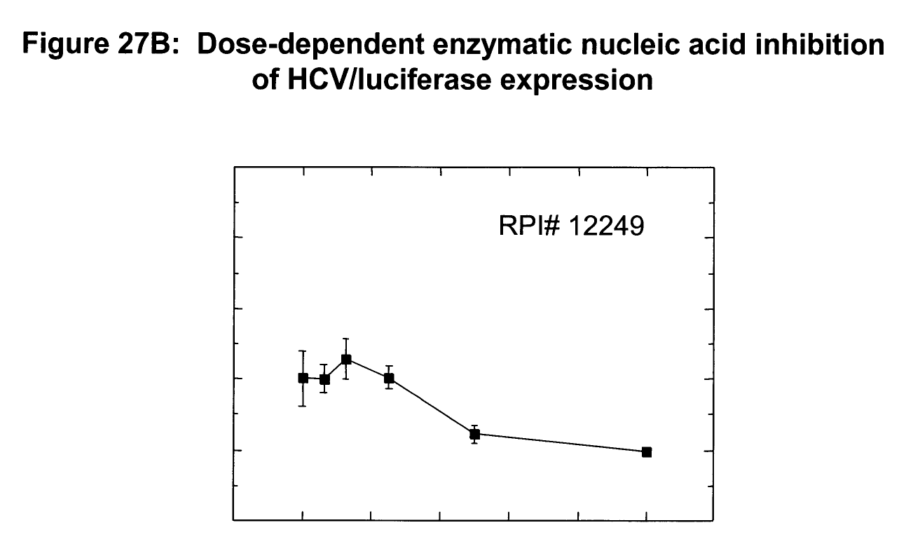

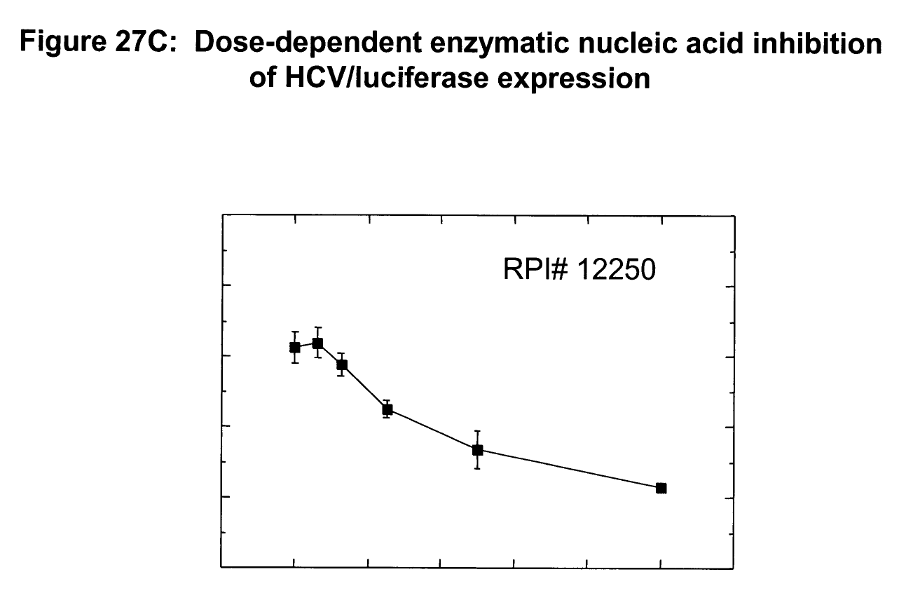

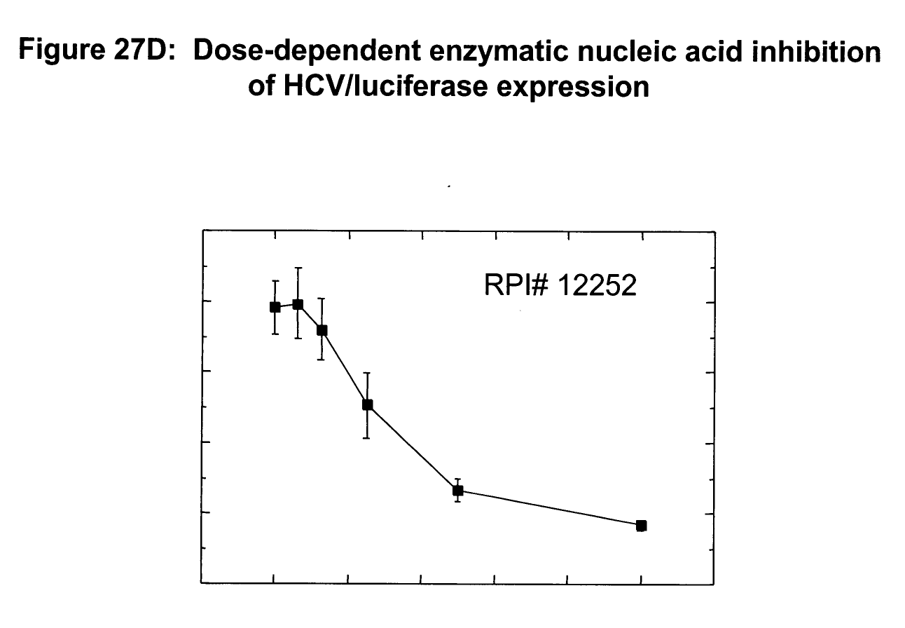

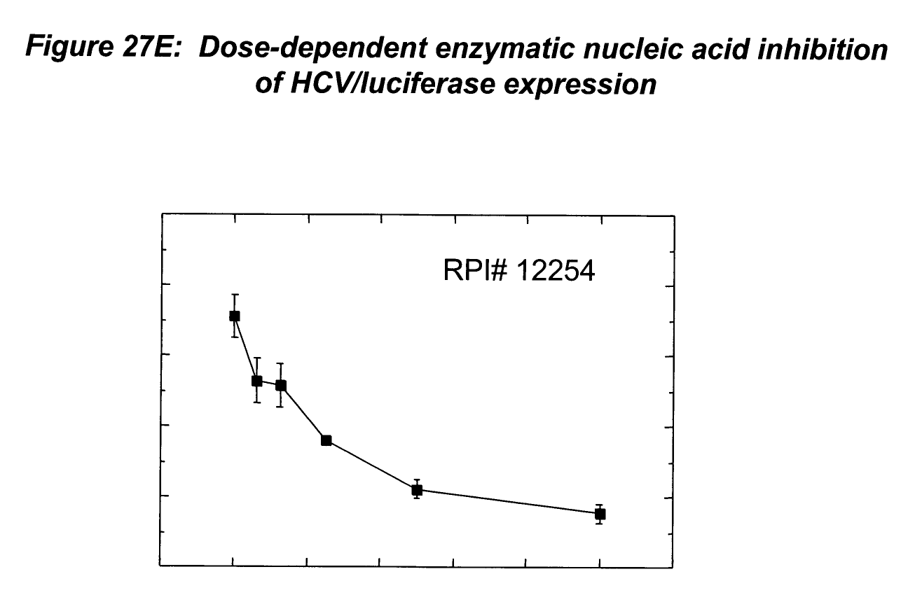

In order to characterize enzymatic nucleic acid efficacy in greater detail, these same 5 lead hammerhead enzymatic nucleic acids were tested for their ability to inhibit HCV/luciferase expression over a range of enzymatic nucleic acid concentrations (0 nM-100 nM). For constant transfection conditions, the total concentration of nucleic acid was maintained at 100 nM for all samples by mixing the active enzymatic nucleic acid with its corresponding AC. Moreover, mixing of active enzymatic nucleic acid and AC maintains the lipid to nucleic acid charge ratio. A concentration-dependent inhibition of HCV/luciferase expression was observed after treatment with each of the 5 enzymatic nucleic acids (FIGS. 27A-E). By linear interpolation, the enzymatic nucleic acid concentration resulting in 50% inhibition (apparent IC₅₀) of HCV/luciferase expression ranged from 40-215 nM. The two most efficacious enzymatic nucleic acids were those designed to cleave after sites 195 or 330 with apparent IC₅₀ values of 46 nM and 40 nM, respectively (FIGS. 27D and E).

### Example 25

### An Enzymatic Nucleic Acid Mechanism is Required for the Observed Inhibition of HCV/Luciferase Expression

To confirm that an enzymatic nucleic acid mechanism of action was responsible for the observed inhibition of HCV/luciferase expression, paired binding-arm attenuated core (BAC) controls (RPI 15291 and 15294) were synthesized for direct comparison to enzymatic nucleic acids targeting sites 195 (RPI 12252) and 330 (RPI 12254). Paired BACs can specifically bind HCV RNA but are unable to promote RNA cleavage because of changes in the catalytic core and, thus, can be used to assess inhibition due to binding alone. Also included in this comparison were paired SAC controls (RPI 15292 and 15295) that contain scrambled binding arms and attenuated catalytic cores, and so lack the ability to bind the target RNA or to catalyze target RNA cleavage.

Enzymatic nucleic acid cleavage of target RNA should result in both a lower level of HCV/luciferase RNA and a subsequent decrease in HCV/luciferase expression. In order to analyze target RNA levels, a reverse transcriptase/polymerase chain reaction (RT-PCR) assay was employed to quantify HCV/luciferase RNA levels. Primers were designed to amplify the luciferase coding region of the HCV 5′UTR/luciferase RNA. This region was chosen because HCV-targeted enzymatic nucleic acids that might co-purify with cellular RNA would not interfere with RT-PCR amplification of the luciferase RNA region. Primers were also designed to amplify the Renilla luciferase RNA so that Renilla RNA levels could be used to control for transfection efficiency and sample recovery.

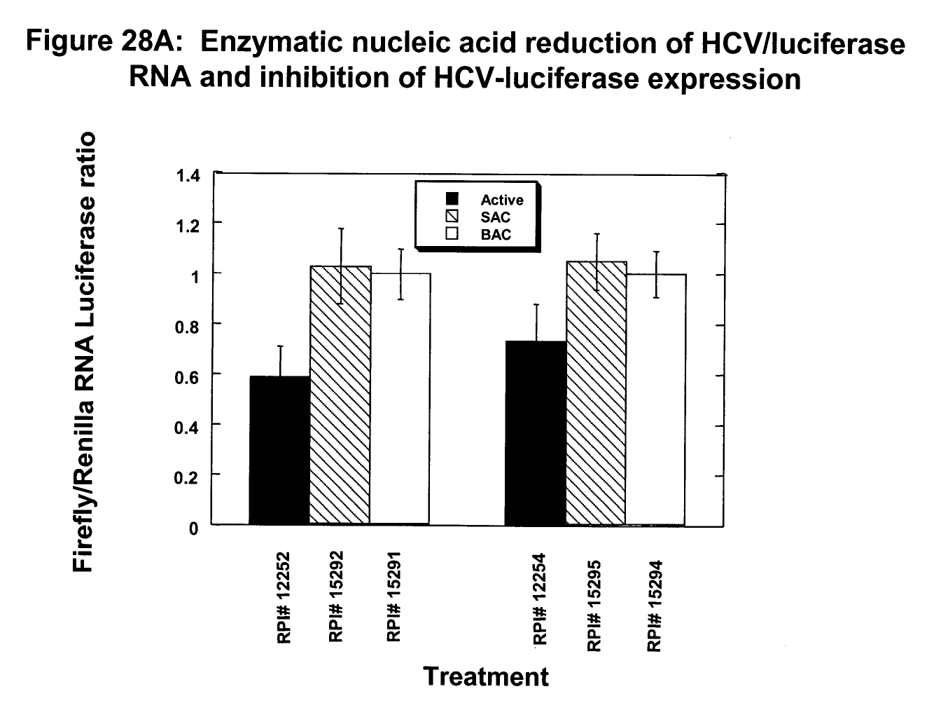

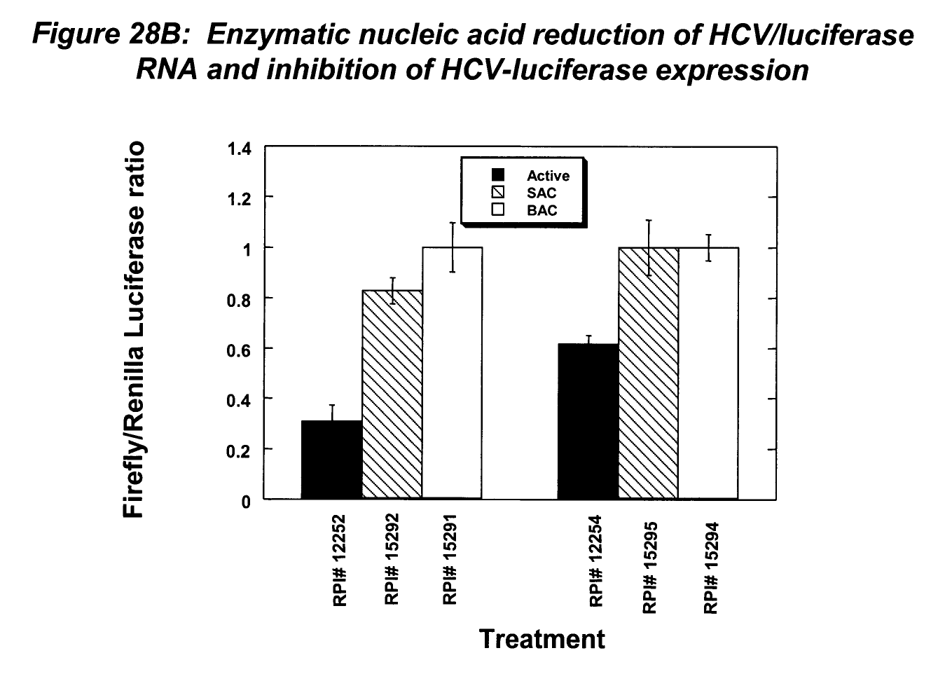

OST7 cells were treated with active enzymatic nucleic acids designed to cleave after sites 195 or 330, paired SACs, or paired BACs. Treatment with enzymatic nucleic acids targeting site 195 or 330 resulted in a significant reduction of HCV/luciferase RNA when compared to their paired SAC controls (P<0.01). In this experiment the site 195 enzymatic nucleic acid was more efficacious than the site 330 enzymatic nucleic acid (FIG. 28A). Treatment with paired BACs that target site 195 or 330 did not reduce HCV/luciferase RNA when compared to the corresponding SACs, thus confirming that the ability to bind alone does not result in a reduction of HCV/luciferase RNA.

To confirm that enzymatic nucleic acid-mediated cleavage of target RNA is necessary for inhibition of HCV/luciferase expression, HCV/luciferase activity was determined in the same experiment. As expected, significant inhibition of HCV/luciferase expression was observed after treatment with active enzymatic nucleic acids when compared to paired SACs (FIG. 28B). Importantly, treatment with paired BACs did not inhibit HCV/luciferase expression, thus confirming that the ability to bind alone is also not sufficient to inhibit translation. As observed in the RNA assay, the site 195 enzymatic nucleic acid was more efficacious than the site 330 enzymatic nucleic acid in this experiment. However, a correlation between enzymatic nucleic acid-mediated HCV RNA reduction and inhibition of HCV/luciferase translation was observed for enzymatic nucleic acids to both sites. The reduction in target RNA and the necessity for an active enzymatic nucleic acid catalytic core confirm that a enzymatic nucleic acid mechanism is required for the observed reduction in HCV/luciferase protein activity in cells treated with site 195 or site 330 enzymatic nucleic acids.

### Example 26

### Zinzyme Inhibition of Chimeric HCV/Poliovirus Replication

During HCV infection, viral RNA is present as a potential target for enzymatic nucleic acid cleavage at several processes: un-coating, translation, RNA replication and packaging. Target RNA can be more or less accessible to enzymatic nucleic acid cleavage at any one of these steps. Although the association between the HCV initial ribosome entry site (IRES) and the translation apparatus is mimicked in the HCV 5′UTR/luciferase reporter system, these other viral processes are not represented in the OST7 system. The resulting RNA/protein complexes associated with the target viral RNA are also absent. Moreover, these processes can be coupled in an HCV-infected cell which could further impact target RNA accessibility. Therefore, applicant tested whether enzymatic nucleic acids designed to cleave the HCV 5′UTR could effect a replicating viral system.

Recently, Lu and Wimmer characterized a HCV-poliovirus chimera in which the poliovirus IRES was replaced by the IRES from HCV (Lu & Wimmer, 1996, Proc. Natl. Acad. Sci. USA. 93, 1412-1417). Poliovirus (PV) is a positive strand RNA virus like HCV, but unlike HCV is non-enveloped and replicates efficiently in cell culture. The HCV-PV chimera expresses a stable, small plaque phenotype relative to wild type PV.

The following enzymatic nucleic acid molecules (zinzymes) were synthesized and tested for replicative inhibition of an HCV/Poliovirus chimera: RPI 18763, RPI 18812, RPI 18749, RPI 18765, RPI 18792, and RPI 18814 (Table XX). A scrambled attenuated core enzymatic nucleic acid, RPI 18743, was used as a control.

HeLa cells were infected with the HCV-PV chimera for 30 minutes and immediately treated with enzymatic nucleic acid. HeLa cells were seeded in U-bottom 96-well plates at a density of 9000-10,000 cells/well and incubated at 37° C. under 5% CO₂ for 24 h. Transfection of nucleic acid (200 nM) was achieved by mixing of 10× nucleic acid (2000 nM) and 10× of a cationic lipid (80 μg/ml) in DMEM (Gibco BRL) with 5% fetal bovine serum (FBS). Nucleic acid/lipid complexes were allowed to incubate for 15 minutes at 37° C. under 5% CO₂. Medium was aspirated from cells and replaced with 80 μl of DMEM (Gibco BRL) with 5% FBS serum, followed by the addition of 20 μls of 10× complexes. Cells were incubated with complexes for 24 hours at 37° C. under 5% CO2.

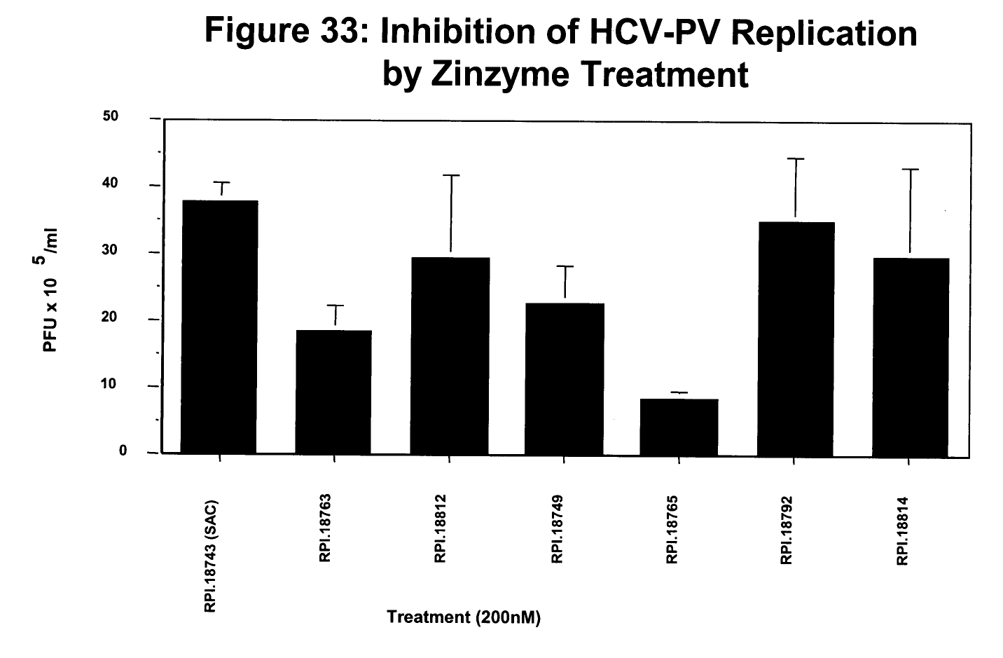

The yield of HCV-PV from treated cells was quantified by plaque assay. The plaque assays were performed by diluting virus samples in serum-free DMEM (Gibco BRL) and applying 100 μl to HeLa cell monolayers (˜80% confluent) in 6-well plates for 30 minutes. Infected monolayers were overlayed with 3 ml 1.2% agar (Sigma) and incubated at 37° C. under 5% CO2. Two or three days later the overlay was removed, monolayers were stained with 1.2% crystal violet, and plaque forming units were counted. The results for the zinzyme inhibition of HCV-PV replication are shown in FIG. 33.

### Example 27

### Antisense Inhibition of Chimeric HCV/Poliovirus Replication

Antisense nucleic acid molecules (RPI 17501 and RPI 17498, Table XXII) were tested for replicative inhibition of an HCV/Poliovirus chimera compared to scrambled controls. An antisense nucleic acid molecule is a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof. Additionally, antisense molecules can be used in combination with the enzymatic nucleic acid molecules of the instant invention.

A RNase H activating region is a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothioate (preferably at least four of the nucleotides are phosphorothiote substitutions; more specifically, 4-11 of the nucleotides are phosphorothiote substitutions); phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

HeLa cells were infected with the HCV-PV chimera for 30 minutes and immediately treated with antisense nucleic acid. HeLa cells were seeded in U-bottom 96-well plates at a density of 9000-10,000 cells/well and incubated at 37° C. under 5% CO₂ for 24 h. Transfection of nucleic acid (200 nM) was achieved by mixing of 10× nucleic acid (2000 nM) and 10× of a cationic lipid (80 μg/ml) in DMEM (Gibco BRL) with 5% fetal bovine serum (FBS). Nucleic acid/lipid complexes were allowed to incubate for 15 minutes at 37° C. under 5% CO₂. Medium was aspirated from cells and replaced with 80 μl of DMEM (Gibco BRL) with 5% FBS serum, followed by the addition of 20 μls of 10× complexes. Cells were incubated with complexes for 24 hours at 37° C. under 5% CO2.

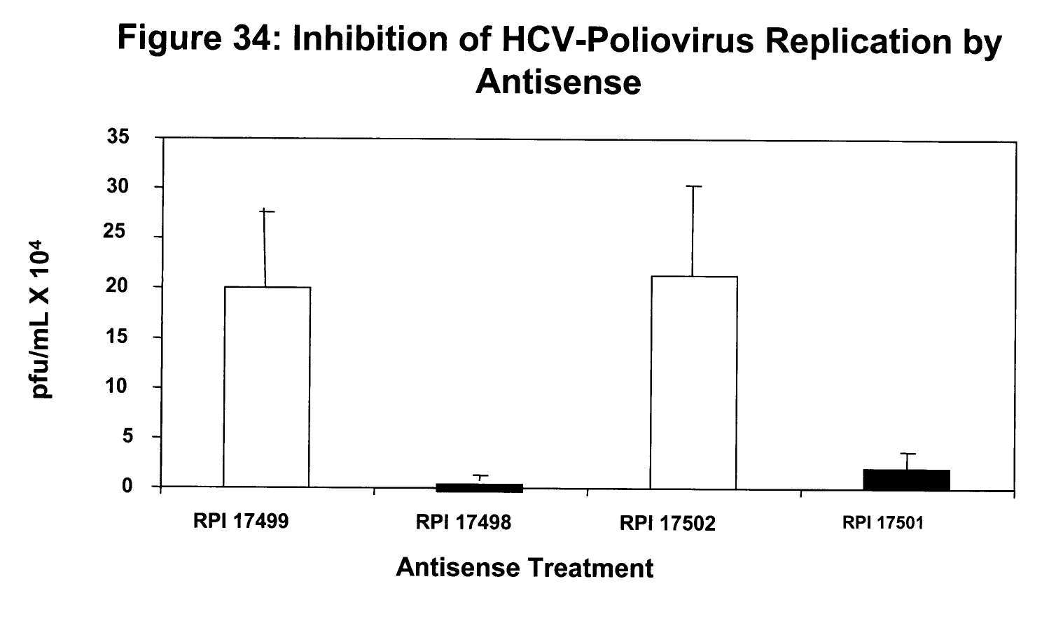

The yield of HCV-PV from treated cells was quantified by plaque assay. The plaque assays were performed by diluting virus samples in serum-free DMEM (Gibco BRL) and applying 100 μl to HeLa cell monolayers (˜80% confluent) in 6-well plates for 30 minutes. Infected monolayers were overlayed with 3 ml 1.2% agar (Sigma) and incubated at 37° C. under 5% CO2. Two or three days later the overlay was removed, monolayers were stained with 1.2% crystal violet, and plaque forming units were counted. The results for the antisense inhibition of HCV-PV are shown in FIG. 34.

### Example 28

### Nucleic Acid Inhibition of Chimeric HCV/PV in Combination with Interferon

One of the limiting factors in interferon (IFN) therapy for chronic HCV are the toxic side effects associated with IFN. Applicant has reasoned that lowering the dose of IFN needed can reduce these side effects. Applicant has previously shown that enzymatic nucleic acid molecules targeting HCV RNA have a potent antiviral effect against replication of an HCV-poliovirus (PV) chimera (Macejak et al., 2000, Hepatology, 31, 769-776). In order to determine if the antiviral effect of type 1 IFN could be improved by the addition of anti-HCV enzymatic nucleic acid treatment, a dose response (0 U/ml to 100 U/ml) with IFN alfa 2a or IFN alfa 2b was performed in HeLa cells in combination with 200 nM site 195 anti-HCV enzymatic nucleic acid (RPI 13919) or enzymatic nucleic acid control (SAC) treatment. The SAC control (RPI 17894) is a scrambled binding arm, attenuated core version of the site 195 enzymatic nucleic acid (RPI 13919). IFN dose responses were performed with different pretreatment regimes to find the dynamic range of inhibition in this system. In these studies, HeLa cells were used instead of HepG2 because of more efficient enzymatic nucleic acid delivery (Macejak et al., 2000, Hepatology, 31, 769-776).

Cells and Virus

HeLa cells were maintained in DMEM (BioWhittaker, Walkersville, Md.) supplemented with 5% fetal bovine serum. A cloned DNA copy of the HCV-PV chimeric virus was a gift of Dr. Eckard Wimmer (NYU, Stony Brook, N.Y.). An RNA version was generated by in vitro transcription and transfected into HeLa cells to produce infectious virus (Lu and Wimmer, 1996, PNAS USA., 93, 1412-1417).

Enzymatic Nucleic Acid Synthesis

Nuclease resistant enzymatic nucleic acids and control oligonucleotides containing 2′-O-methyl-nucleotides, 2′-deoxy-2′-C-allyl uridine, a 3′-inverted abasic cap, and phosphorothioate linkages were chemically synthesized. The anti-HCV enzymatic nucleic acid (RPI 13919) targeting cleavage after nucleotide 195 of the 5′ UTR of HCV is shown in Table XX. Attenuated core controls have nucleotide changes in the core sequence that greatly diminished the enzymatic nucleic acid's cleavage activity. The attenuated controls either contain scrambled binding arms (referred to as SAC, RPI 18743) or maintain binding arms (BAC, RPI 17894) capable of binding to the HCV RNA target.

Enzymatic Nucleic Acid Delivery

A cationic lipid was used as a cytofectin agent. HeLa cells were seeded in 96-well plates at a density of 9000-10,000 cells/well and incubated at 37° C. under 5% CO₂ for 24 h. Transfection of enzymatic nucleic acid or control oligonucleotides (200 nM) was achieved by mixing 10× enzymatic nucleic acid or control oligonucleotides (2000 nM) with 10× RPI.9778 (80 μg/ml) in DMEM containing 5% fetal bovine serum (FBS) in U-bottom 96-well plates to make 5× complexes. Enzymatic nucleic acid/lipid complexes were allowed to incubate for 15 min at 37° C. under 5% CO2. Medium was aspirated from cells and replaced with 80 μl of DMEM (Gibco BRL) containing 5% FBS serum, followed by the addition of 20 μl of 5× complexes. Cells were incubated with complexes for 24 h at 37° C. under 5% CO2.

Interferon/Enzymatic Nucleic Acid Combination Treatment

Interferon alfa 2a (Roferon®) was purchased from Roche Bioscience (Palo Alto, Calif.). Interferon alfa 2b (Intron A®) was purchased from Schering-Plough Corporation (Madison, N.J.). Consensus interferon (interferon-alfa-con 1) was a generous gift of Amgen, Inc. (Thousand Oaks, Calif.). For the basis of comparison, the manufacturers' specified units were used in the studies reported here; however, the manufacturers' unit definitions of these three IFN preparations are not necessarily the same. Nevertheless, since clinical dosing is based on the manufacturers' specified units, a direct comparison based on these units has relevance to clinical therapeutic indices. HeLa cells were seeded (10,000 cells per well) and incubated at 37° C. under 5% CO2 for 24 h. Cells were then pre-treated with interferon in complete media (DMEM+5% FBS) for 4 h and then infected with HCV-PV at a multiplicity of infection (MOI)=0.1 for 30 min. The viral inoculum was then removed and enzymatic nucleic acid or attenuated control (SAC or BAC) was delivered with the cytofectin formulation (8 μg/ml) in complete media for 24 h as described above. Where indicated for enzymatic nucleic acid dose response studies, active enzymatic nucleic acid was mixed with SAC to maintain a 200 nM total oligonucleotide concentration and the same lipid charge ratio. After 24 h, cells were lysed to release virus by three cycles of freeze/thaw. Virus was quantified by plaque assay and viral yield is reported as mean plaque forming units per ml (pfu/ml)+SD. All experiments were repeated at least twice and the trends in the results reported were reproducible. Significance levels (P values) were determined by the Student's test.

Plaque Assay

Virus samples were diluted in serum-free DMEM and 100 μl applied to Vero cell monolayers (˜80% confluent) in 6-well plates for 30 min. Infected monolayers were overlaid with 3 ml 1.2% agar (Sigma Chemical Company, St. Louis, Mo.) and incubated at 37° C. under 5% CO2. When plaques were visible (after two to three days) the overlay was removed, monolayers were stained with 1.2% crystal violet, and plaque forming units were counted.

Results

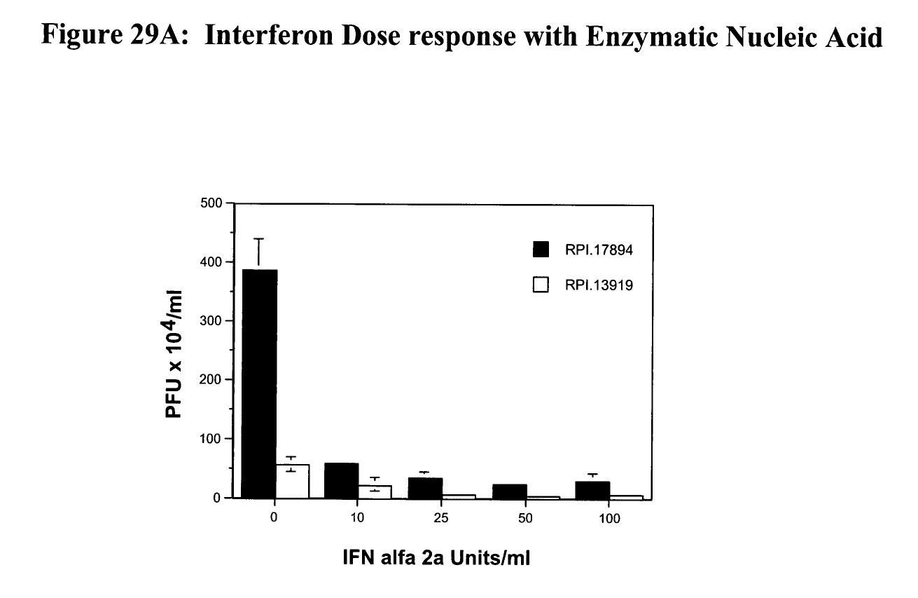

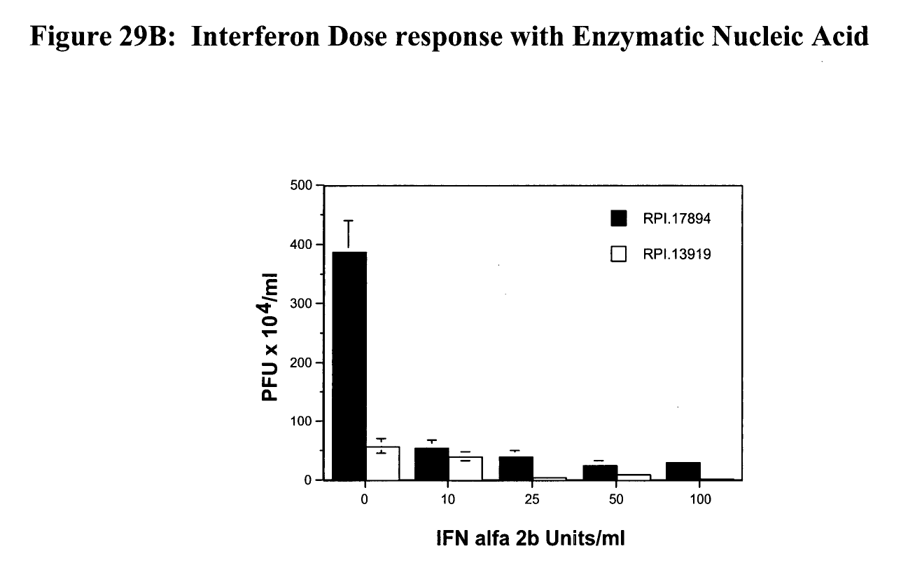

As shown in FIGS. 29A and 29B, treatment with the site 195 (RPI 13919) anti-HCV hammerhead enzymatic nucleic acid alone (0 U/ml IFN) resulted in viral replication that was dramatically reduced compared to SAC-treated cells (85%, P<0.01). For both IFN alfa 2a (FIG. 29A) or IFN alfa 2b (FIG. 29B), treatment with 25 U/ml resulted in a ˜90% inhibition of HCV-PV replication in SAC-treated cells as compared to cells treated with SAC alone (p<0.01 for both observations). The maximal level of inhibition in SAC-treated cells (94%) was achieved by treatment with >50 U/ml of either IFN alfa 2a or IFN alfa 2b (p<0.01 for both observations versus SAC alone). Maximal inhibition could however, be achieved by a 5-fold lower dose of IFN alfa 2a (10 U/ml) if enzymatic nucleic acid targeting site 195 in the 5′ UTR of HCV RNA was given in combination (FIG. 29A, p<0.01). While the additional effect of enzymatic nucleic acid treatment on IFN alfa 2b-treated cells at 10 U/ml was very slight, the combined effect with 25 U/ml IFN alfa 2b was greater in magnitude (FIG. 29B). For both interferons tested, pretreatment with 25 U/ml in combination with 200 nM site 195 anti-HCV enzymatic nucleic acid resulted in an even greater level of inhibition of viral replication (>98%) compared to replication in cells treated with 200 nM SAC alone (P<0.01).

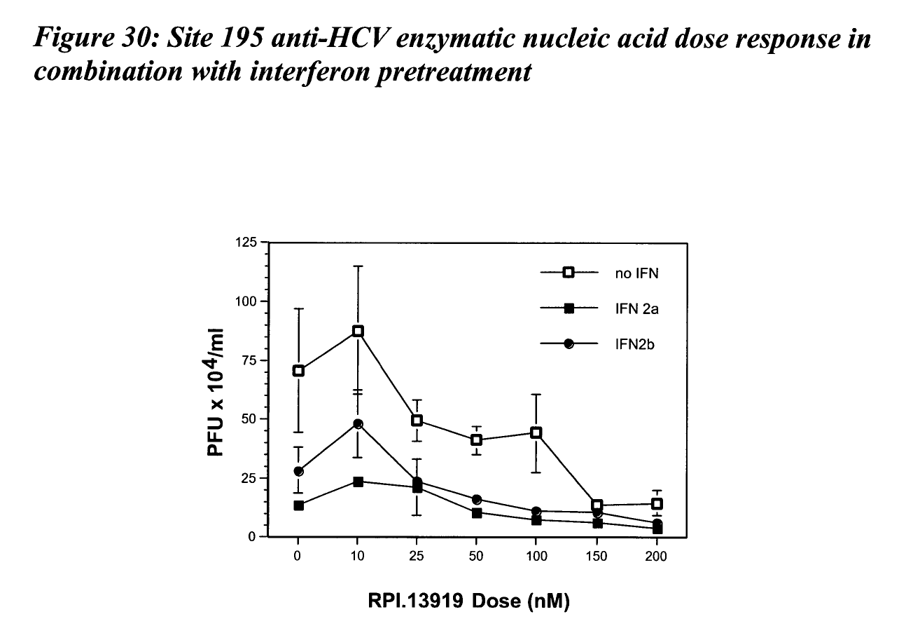

A dose response of the site 195 anti-HCV enzymatic nucleic acid was also performed in HeLa cells, either with or without 12.5 U/ml IFN alfa 2a or IFN alfa 2b pretreatment. As shown in FIG. 30, enzymatic nucleic acid-mediated inhibition was dose-dependent and a significant inhibition of HCV-PV replication (>75% versus 0 nM enzymatic nucleic acid, P<0.01) could be achieved by treatment with >150 nM anti-HCV enzymatic nucleic acid alone (no IFN). However, in IFN-pretreated cells, the dose of anti-HCV enzymatic nucleic acid needed to achieve this level of inhibition was decreased 3-fold to 50 nM (P<0.01 versus 0 nM enzymatic nucleic acid). In comparison, treatment with the site 195 anti-HCV enzymatic nucleic acid alone at 50 nM resulted in only 40% inhibition of virus replication. Pretreatment with IFN enhanced the antiviral effect of site 195 enzymatic nucleic acid at all enzymatic nucleic acid doses, compared to no IFN pretreatment.

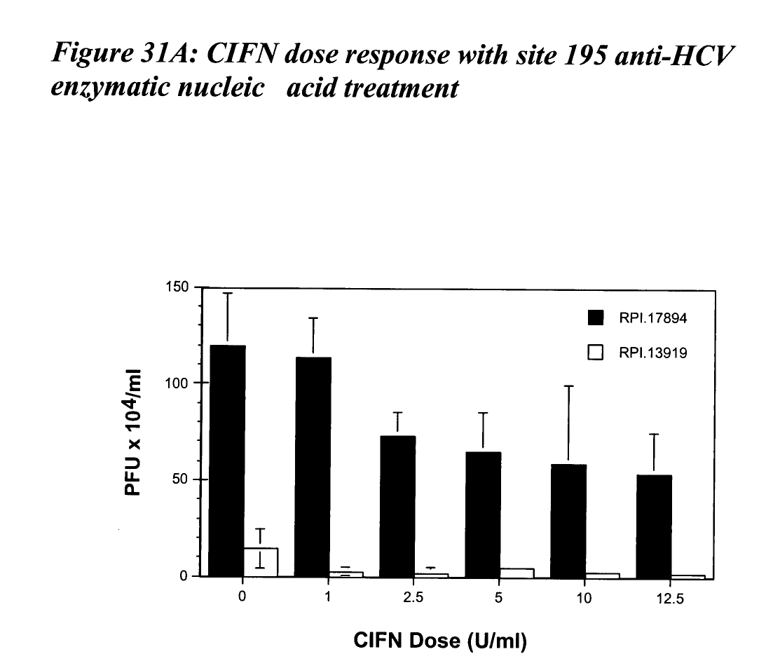

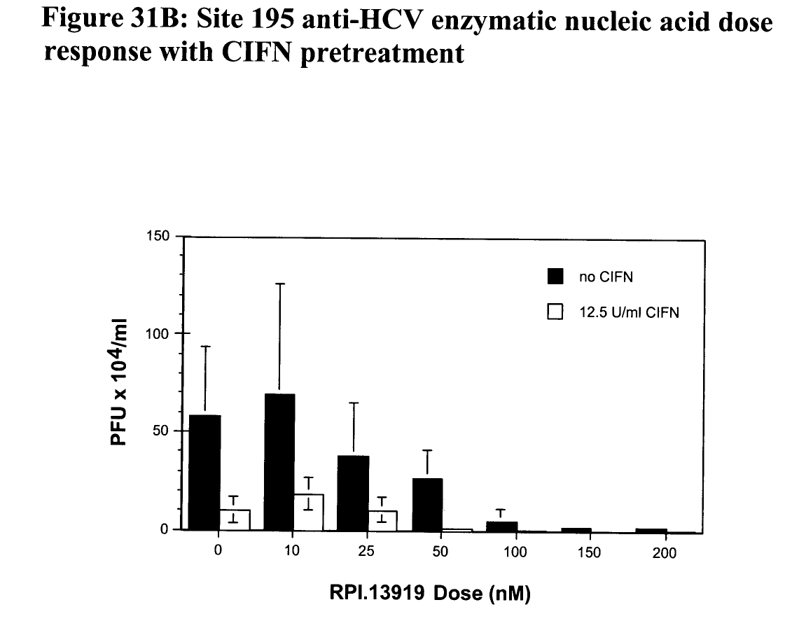

Interferon-alfacon1, consensus IFN (CIFN), is another type 1 IFN that is used to treat chronic HCV. To determine if a similar enhancement can occur in CIFN-treated cells, a dose response with CIFN was performed in HeLa cells using 0 U/ml to 12.5 U/ml CIFN in combination with 200 nM site 195 anti-HCV enzymatic nucleic acid or SAC treatment (FIG. 31A). Again, in the presence of the site 195 anti-HCV enzymatic nucleic acid alone, viral replication was dramatically reduced compared to SAC-treated cells. As shown in FIG. 31A, treatment with 200 mM anti-HCV enzymatic nucleic acid alone significantly inhibited HCV-PV replication (90% versus SAC treatment, P<0.01). However, pretreatment with concentrations of CIFN from 1 U/ml to 12.5 U/ml in combination with 200 nM anti-HCV enzymatic nucleic acid resulted in even greater inhibition of viral replication (>98%) compared to replication in cells treated with 200 nM SAC alone (P<0.01). It is important to note that pretreatment with 1 U/ml CIFN in SAC-treated cells did not have a significant effect on HCV-poliovirus replication, but in the presence of enzymatic nucleic acid a significant inhibition of replication was observed (>98%, P<0.01). Thus, the dose of CIFN needed to achieve a >98% inhibition could be lowered to 1 U/ml in cells also treated with 200 nM site 195 anti-HCV enzymatic nucleic acid.

A dose response of site 195 anti-HCV enzymatic nucleic acid was then performed in HeLa cells, either with or without 12.5 U/ml CIFN pretreatment. As shown in FIG. 31B, a significant inhibition of HCV-PV replication (>95% versus 0 mM enzymatic nucleic acid, P<0.01) could be achieved by treatment with ≧150 nM anti-HCV enzymatic nucleic acid alone. However, in CIFN-pretreated cells, the dose of anti-HCV enzymatic nucleic acid needed to achieve this level of inhibition was only 50 nM (P<0.01). In comparison, treatment with the site 195 anti-HCV enzymatic nucleic acid alone at 50 nM resulted in ˜50% inhibition of virus replication. Thus, as was seen with IFN alfa 2a and IFN alfa 2b, the dose of enzymatic nucleic acid could be reduced 3-fold in the presence of CIFN pretreatment to achieve a similar antiviral effect as enzymatic nucleic acid-treatment alone.

To further explore the combination of lower enzymatic nucleic acid concentration and CIFN, a dose response with 0 U/ml to 12.5 U/ml CIFN was subsequently performed in HeLa cells in combination with 50 nM site 195 anti-HCV enzymatic nucleic acid treatment. In multiple experiments, treatment with 50 nM anti-HCV enzymatic nucleic acid alone inhibited HCV-PV replication 50%-81% compared to viral replication in SAC-treated cells. As for the experiment shown in FIG. 31A, treatment with CIFN alone at 5 U/ml resulted in ˜50% inhibition of viral replication. However, a four hour pretreatment with 5 U/ml CIFN followed by 50 nM anti-HCV enzymatic nucleic acid treatment resulted in 95%-97% inhibition compared to SAC-treated cells (P<0.01).

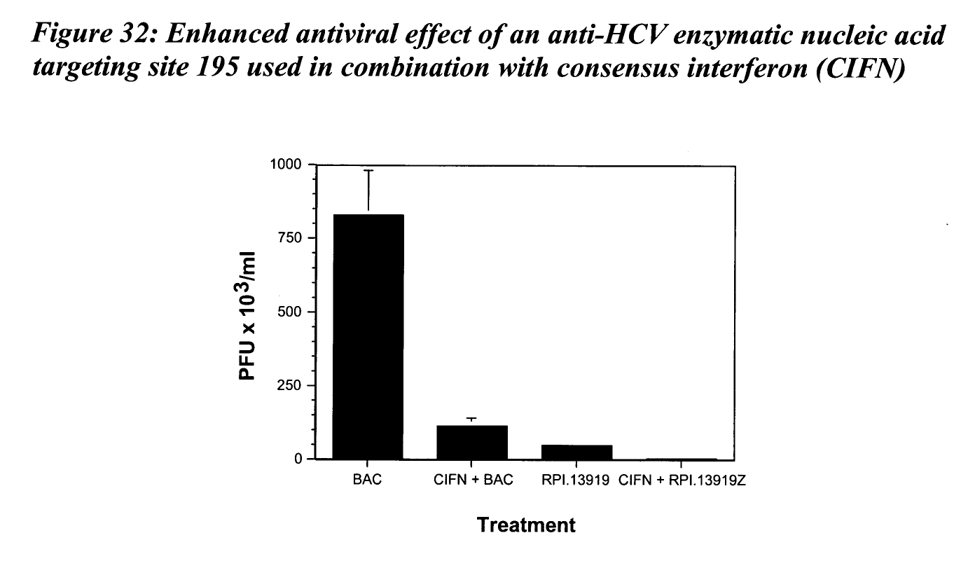

To demonstrate that the enhanced antiviral effect of CIFN and enzymatic nucleic acid combination treatment was dependent upon enzymatic nucleic acid cleavage activity, the effect of CIFN in combination with site 195 anti-HCV enzymatic nucleic acid versus the effect of CIFN in combination with a binding competent, attenuated core, control (BAC) was then compared. The BAC can still bind to its specific RNA target, but is greatly diminished in cleavage activity. Pretreatment with 12.5 U/ml CIFN reduced the viral yield −90% (7-fold) in cells treated with BAC (compare CIFN versus BAC in FIG. 32). Cells treated with 200 nM site 195 anti-HCV enzymatic nucleic acid alone produced −95% (17-fold) less virus than BAC-treated cells (195 RZ BAC in FIG. 32). The combination of CIFN pretreatment and 200 nM site 195 anti-HCV enzymatic nucleic acid results in an augmented >98% (300-fold) reduction in viral yield (CIFN+RZ versus control in FIG. 32).

2′-5′-Oligoadenylate Inhibition of HCV

Type 1 Interferon is a key constituent of many effective treatment programs for chronic HCV infection. Treatment with type 1 interferon induces a number of genes and results in an antiviral state within the cell. One of the genes induced is 2′,5′ oligoadenylate synthetase, an enzyme that synthesizes short 2′,5′ oligoadenylate (2-5A) molecules. Nascent 2-5A subsequently activates a latent RNase, RNase L, which in turn nonspecifically degrades viral RNA. As described herein, ribozymes targeting HCV RNA that inhibit the replication of an HCV-poliovirus (HCV-PV) chimera in cell culture and have shown that this antiviral effect is augmented if ribozyme is given in combination with type 1 interferon. In addtion, the 2-5A component of the interferon response can also inhibit replication of the HCV-PV chimera.

The antiviral effect of anti-HCV ribozyme treatment is enhanced if type 1 interferon is given in combination. Interferon induces a number of gene products including 2′,5′ oligoadenylate (2-5A) synthetase, double-stranded RNA-activated protein kinase (PKR), and the Mx proteins. Mx proteins appear to interfere with nuclear transport of viral complexes and are not thought to play an inhibitory role in HCV infection. On the other hand, the additional 2-5A-mediated RNA degradation (via RNase L) and/or the inhibition of viral translation by PKR in interferon-treated cells can augment the ribozyme-mediated inhibition of HCV-PV replication.

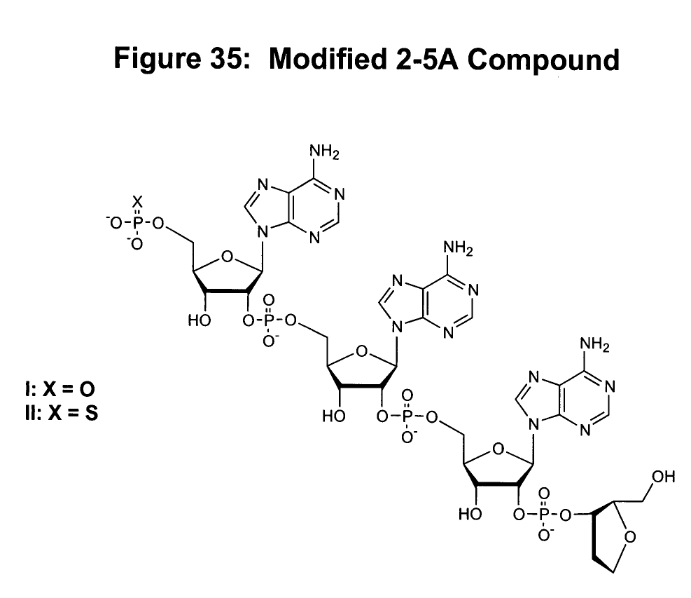

To investigate the potential role of the 2-5A/RNase L pathway in this enhancement phenomenon, HCV-PV replication was analyzed in HeLa cells treated exogenously with chemically-synthesized analogs of 2-5A (FIG. 35), alone and in combination with the anti-HCV ribozyme (RPI 13919). These results were compared to replication in cells treated with interferon and/or anti-HCV ribozyme. Anti-HCV ribozyme was transfected into cells with a cationic lipid. To control for nonspecific effects due to lipid-mediated transfection, a scrambled arm, attenuated core, oligonucleotide (SAC) (RPI 17894) was transfected for comparison. The SAC is the same base composition as the ribozyme but is greatly attenuated in catalytic activity due to changes in the core sequence and cannot bind specifically to the HCV sequence.

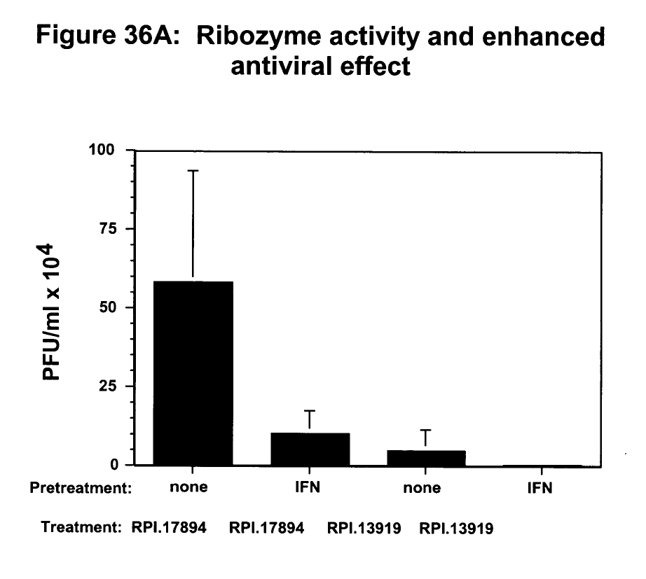

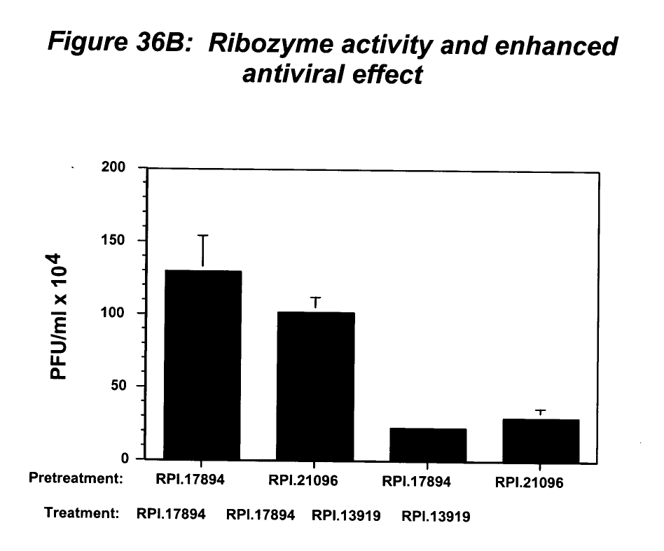

As shown in FIG. 36A, HeLa cells pretreated with 10 U/ml consensus interferon for 4 hours prior to HCV-PV infection resulted in ˜70% reduction of viral replication in SAC-treated cells. Similarly, HeLa cells treated with 100 nM anti-HCV ribozyme for 20 hours after infection resulted in an ˜80% reduction in viral yield. This antiviral effect was enhanced to ˜98% inhibition in HeLa cells pretreated with interferon for 4 hours before infection and then treated with anti-HCV ribozyme for 20 hours after infection. In parallel, a 2-5A compound (analog I, FIG. 35) that was protected from nuclease digestion at the 3′-end with an inverted abasic moiety was tested. As shown in FIG. 36B, treatment with 200 nM 2-5A analog I for 4 hours prior to HCV-PV infection only slightly inhibited HCV-PV replication (˜20%) in SAC-treated cells. Moreover, the inhibition due to a 20 hour anti-HCV ribozyme treatment was not augmented with a 4 hour pretreatment of 2-5A in combination (compare third bar to fourth bar in FIG. 36B).

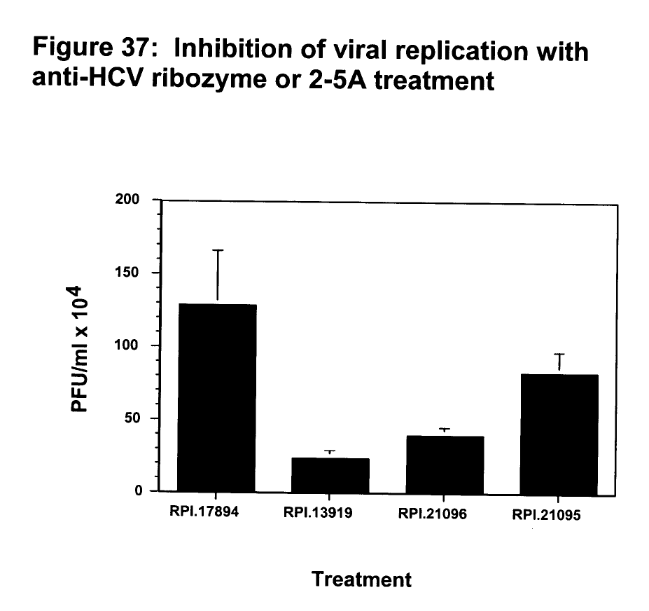

There are several possible possible explanations why the chemically synthesized 2-5A analog was not able to completely activate RNase L. It is possible that the 2-5A analog was not sufficiently stable or that in this experiment the 4 hour pretreatment period was too short for RNase L activation. To test these possibilities, a 2-5A compound containing a 5′-terminal thiophosphate (P═S) for added nuclease resistance, in addition to the 3′-abasic, was also included (analog II, FIG. 35). In addition, a longer 2-5A treatment was used. In this experiment (FIG. 37), HeLa cells were treated with 2-5A or 2-5A (P═S) for 20 hours after HCV-PV infection. Again, anti-HCV ribozyme treatment resulted in >80% inhibition. In contrast to the 20% inhibition of viral replication seen with a 4 hour 2-5A pretreatment, viral replication in cells treated with 2-5A analog I for 20 hours after HCV-PV infection was inhibited by ˜70%. The P═S version (analog II) inhibited HCV-PV replication by −35%. Thus, both 2-5A analogs used here are able to generate an antiviral effect, presumably through RNase L activation. The P═S version, although more resistant to 5′ dephosphorylation, did not yield as great an anti-viral effect. It is possible that combination of the 5′-terminal thiophosphate together with the presence of a 3′-inverted abasic moiety can interfere with RNase L activation. Nevertheless, these results demonstrate potent anti-HCV activity by a nuclease-stabilized 2-5A analog.

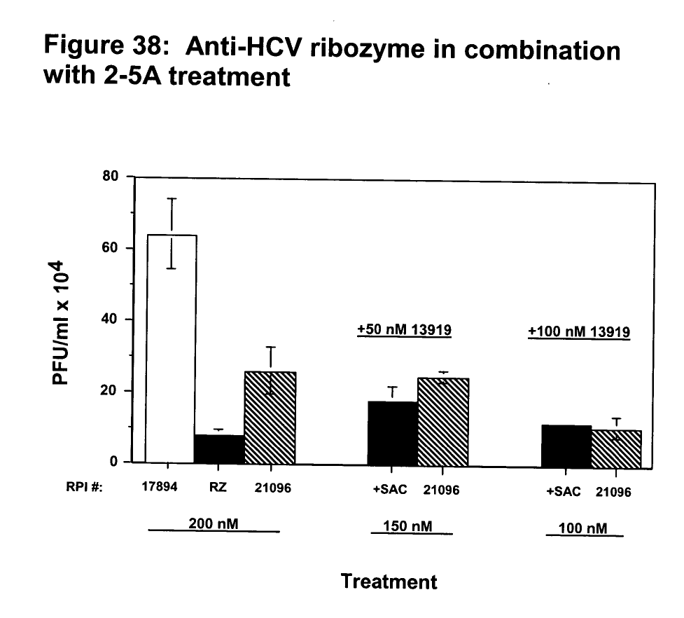

The level of reduction in HCV-PV replication in cells treated with 2-5A analog I for 20 hours was similar to that in cells pretreated with consensus interferon for 4 hours. To determine if this expanded 2-5A treatment regimen would enhance anti-HCV ribozyme efficacy to the same degree as does the interferon pretreatment, HeLa cells infected with HCV-PV were treated with a combination of 2-5A and anti-HCV ribozyme for 20 hours after infection. In this experiment, a 200 mM treatment with anti-HCV ribozyme or 2-5A treatment alone inhibited viral replication by 88% or ˜60%, respectively, compared to SAC treatment (FIG. 38, left three bars). To maintain consistent transfection conditions but vary the concentration of anti-HCV ribozyme or 2-5A, anti-HCV ribozyme was mixed with the SAC to maintain a total dose of 200 nM. A 50 mM treatment with anti-HCV ribozyme inhibited HCV-PV replication by ˜70% (solid middle bar). However, the amount of HCV-PV replication was not further reduced in cells treated with a combination of 50 nM anti-HCV ribozyme and 150 nM 2-5A (striped middle bar). Likewise, cells treated with 100 nM anti-HCV ribozyme inhibited HCV-PV replication by ˜80% whether they were also treated with 100 mM of 2-5A or SAC (right two bars). In contrast, antiviral activity increased from 80% to 98% when 100 nM anti-HCV ribozyme was given in combination with interferon (FIG. 36A). The reasons for the lack of additive or synergistic effects for the ribozyme/2-5A combination therapy is unclear at this time but can be due to that fact that both compounds have a similar mechanism of action (degradation of RNA). Further study is warranted to examine this possibility.

As a monotherapy, 2-5A treatment generates a similar inhibitory effect on HCV-poliovirus replication as does interferon treatment. If these results are maintained in HCV patients, treatment with 2-5A can not only be efficacious but can also generate less side effects than those observed with interferon if the plethora of interferon-induced genes were not activated.

HBV Cell Culture Models

As previously mentioned, HBV does not infect cells in culture. However, transfection of HBV DNA (either as a head-to-tail dimer or as an “overlength” genome of >100%) into HuH7 or Hep G2 hepatocytes results in viral gene expression and production of HBV virions released into the media. Thus, HBV replication competent DNA are co-transfected with ribozymes in cell culture. Such an approach has been used to report intracellular ribozyme activity against HBV (zu Putlitz, et al., 1999, J. Virol., 73, 5381-5387, and Kim et al., 1999, Biochem. Biophys. Res. Commun., 257, 759-765). In addition, stable hepatocyte cell lines have been generated that express HBV. In these cells, only ribozyme need be delivered; however, performance of a delivery screen is required. Intracellular HBV gene expression can be assayed by a Taqman® assay for HBV RNA or by ELISA for HBV protein. Extracellular virus can be assayed by PCR for DNA or ELISA for protein. Antibodies are commercially available for HBV surface antigen and core protein. A secreted alkaline phosphatase expression plasmid can be used to normalize for differences in transfection efficiency and sample recovery.

HBV Animal Models

There are several small animal models to study HBV replication. One is the transplantation of HBV-infected liver tissue into irradiated mice. Viremia (as evidenced by measuring HBV DNA by PCR) is first detected 8 days after transplantation and peaks between 18-25 days (Ilan et al., 1999, Hepatology, 29, 553-562).

Transgenic mice that express HBV have also been used as a model to evaluate potential anti-virals. HBV DNA is detectable in both liver and serum (Guidotti et al., 1995, J. Virology, 69, 10, 6158-6169; Morrey et al., 1999, Antiviral Res., 42, 97-108).

An additional model is to establish subcutaneous tumors in nude mice with Hep G2 cells transfected with HBV. Tumors develop in about 2 weeks after inoculation and express HBV surface and core antigens. HBV DNA and surface antigen is also detected in the circulation of tumor-bearing mice (Yao et al., 1996, J. Viral Hepat., 3, 19-22).

In one embodiment, the invention features a mouse, for example a male or female mouse, implanted with HepG2.2.15 cells, wherein the mouse is susceptible to HBV infection and capable of sustaining HBV DNA expression. One embodiment of the invention provides a mouse implanted with HepG2.2.15 cells, wherein said mouse sustains the propagation of HEPG2.2.15 cells and HBV production (see Macejak, U.S. Provisional Patent Application No. 60/296,876).

Woodchuck hepatitis virus (WHV) is closely related to HBV in its virus structure, genetic organization, and mechanism of replication. As with HBV in humans, persistent WHV infection is common in natural woodchuck populations and is associated with chronic hepatitis and hepatocellular carcinoma (HCC). Experimental studies have established that WHV causes HCC in woodchucks and woodchucks chronically infected with WHV have been used as a model to test a number of anti-viral agents. For example, the nucleoside analogue 3T3 was observed to cause dose dependent reduction in virus (50% reduction after two daily treatments at the highest dose) (Hurwitz et al., 1998. Antimicrob. Agents Chemother., 42, 2804-2809).

HCV Cell Culture Models

Although there have been reports of replication of HCV in cell culture (see below), these systems are difficult to replicate and have proven unreliable. Therefore, as was the case for development of other anti-HCV therapeutics such as interferon and ribavirin, after demonstration of safety in animal studies applicant can proceed directly into a clinical feasibility study.

Several recent reports have documented in vitro growth of HCV in human cell lines (Mizutani et al, Biochem Biophys Res Commun 1996 227(3):822-826; Tagawa et al., Journal of Gasteroenterology and Hepatology 1995 10(5):523-527; Cribier et al., Journal of General Virology 76(10):2485-2491; Seipp et al., Journal of General Virology 1997 78(10)2467-2478; lacovacci et al., Research Virology 1997 148(2):147-151; locavacci et al., Hepatology 1997 26(5) 1328-1337; Ito et al., Journal of General Virology 1996 77(5):1043-1054; Nakajima et al., Journal of Virology 1996 70(5):3325-3329; Mizutani et al., Journal of Virology 1996 70(10):7219-7223; Valli et al., Res Virol 1995 146(4): 285-288; Kato et al., Biochem Biophys Res Comm 1995 206(3):863-869). Replication of HCV has been demonstrated in both T and B cell lines as well as cell lines derived from human hepatocytes. Demonstration of replication was documented using either RT-PCR based assays or the b-DNA assay. It is important to note that the most recent publications regarding HCV cell cultures document replication for up to 6-months.

Additionally, another recent study has identified more robust strains of hepatitis C virus having adaptive mutations that allow the strains to replicate more vigorously in human cell cult. The mutations that confer this enhanced ability to replicate are located in a specific region of a protein identified as NS5A. Studies performed at Rockefeller University have shown that in certain cell culture systems, infection with the robust strains produces a 10,000-fold increase in the number of infected cells. The greatly increased availability of HCV-infected cells in culture can be used to develop high-throughput screening assays, in which a large number of compounds, such as enzymatic nucleic acid molecules, can be tested to determine their effectiveness.

In addition to cell lines that can be infected with HCV, several groups have reported the successful transformation of cell lines with cDNA clones of full-length or partial HCV genomes (Harada et al., Journal of General Virology 1995 76(5)1215-1221; Haramatsu et al., Journal of Viral Hepatitis 1997 4S(1):61-67; Dash et al., American Journal of Pathology 1997 151(2):363-373; Mizuno et al., Gasteroenterology 1995 109(6):1933-40; Yoo et al., Journal Of Virology 1995 69(1):32-38).

HCV Animal Models

The best characterized animal system for HCV infection is the chimpanzee. Moreover, the chronic hepatitis that results from HCV infection in chimpanzees and humans is very similar. Although clinically relevant, the chimpanzee model suffers from several practical impediments that make use of this model difficult. These include; high cost, long incubation requirements and lack of sufficient quantities of animals. Due to these factors, a number of groups have attempted to develop rodent models of chronic hepatitis C infection. While direct infection has not been possible several groups have reported on the stable transfection of either portions or entire HCV genomes into rodents (Yamamoto et al., Hepatology 1995 22(3): 847-855; Galun et al., Journal of Infectious Disease 1995 172(1):25-30; Koike et al., Journal of general Virology 1995 76(12)3031-3038; Pasquinelli et al., Hepatology 1997 25(3): 719-727; Hayashi et al., Princess Takamatsu Symp 1995 25:1430149; Mariya K, Yotsuyanagi H, Shintani Y, Fujie H, Ishibashi K, Matsuura Y, Miyamura T, Koike K. Hepatitis C virus core protein induces hepatic steatosis in transgenic mice. Journal of General Virology 1997 78(7) 1527-1531; Takehara et al., Hepatology 1995 21(3):746-751; Kawamura et al., Hepatology 1997 25(4): 1014-1021). In addition, transplantation of HCV infected human liver into immunocompromised mice results in prolonged detection of HCV RNA in the animal's blood.

Vierling, International PCT Publication No. WO 99/16307, describes a method for expressing hepatitis C virus in an in vivo animal model. Viable, HCV infected human hepatocytes are transplanted into a liver parenchyma of a scid/scid mouse host. The scid/scid mouse host is then maintained in a viable state, whereby viable, morphologically intact human hepatocytes persist in the donor tissue and hepatitis C virus is replicated in the persisting human hepatocytes. This model provides an effective means for the study of HCV inhibition by enzymatic nucleic acids in vivo.

Indications

Particular degenerative and disease states that can be associated with HBV expression modulation include, but are not limited to, HBV infection, hepatitis, cancer, tumorigenesis, cirrhosis, liver failure and other conditions related to the level of HBV.

Particular degenerative and disease states that can be associated with HCV expression modulation include, but are not limited to, HCV infection, hepatitis, cancer, tumorigenesis, cirrhosis, liver failure and other conditions related to the level of HCV.

The present body of knowledge in HBV and HCV research indicates the need for methods to assay HBV or HCV activity and for compounds that can regulate HBV and HCV expression for research, diagnostic, and therapeutic use.

Lamivudine (3TC®), L-FMAU, adefovir dipivoxil, type 1 Interferon (e.g, interferon alpha, interferon beta, consensus interferon, polyethylene glycol interferon, polyethylene glycol interferon alpha 2a, polyethylene glycol interferon 2b, and polyethylene glycol consensus interferon), therapeutic vaccines, steriods, and 2′-5′ Oligoadenylates are non-limiting examples of pharmaceutical agents that can be combined with or used in conjunction with the nucleic acid molecules (e.g. ribozymes and antisense molecules) of the instant invention. Those skilled in the art will recognize that other drugs or other therapies can similarly and readily be combined with the nucleic acid molecules of the instant invention (e.g. ribozymes and antisense molecules) and are, therefore, within the scope of the instant invention.

Diagnostic Uses

The nucleic acid molecules of this invention can be used as diagnostic tools to examine genetic drift and mutations within diseased cells or to detect the presence of HBV or HCV RNA in a cell. For example, the close relationship between enzymatic nucleic acid activity and the structure of the target RNA allows the detection of mutations in any region of the molecule which alters the base-pairing and three-dimensional structure of the target RNA. By using multiple enzymatic nucleic acids described in this invention, one can map nucleotide changes which are important to RNA structure and function in vitro, as well as in cells and tissues. Cleavage of target RNAs with enzymatic nucleic acids can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g., multiple enzymatic nucleic acid molecules targeted to different genes, enzymatic nucleic acid molecules coupled with known small molecule inhibitors, or intermittent treatment with combinations of enzymatic nucleic acid molecules and/or other chemical or biological molecules). Other in vitro uses of enzymatic nucleic acid molecules of this invention are well known in the art, and include detection of the presence of mRNAs associated with HBV or HCV-related condition. Such RNA is detected by determining the presence of a cleavage product after treatment with an enzymatic nucleic acid using standard methodology.

In a specific example, enzymatic nucleic acid molecules which can cleave only wild-type or mutant forms of the target RNA are used for the assay. The first enzymatic nucleic acid is used to identify wild-type RNA present in the sample and the second enzymatic nucleic acid is used to identify mutant RNA in the sample. As reaction controls, synthetic substrates of both wild-type and mutant RNA can be cleaved by both enzymatic nucleic acid molecules to demonstrate the relative ribozyme efficiencies in the reactions and the absence of cleavage of the “non-targeted” RNA species. The cleavage products from the synthetic substrates can also serve to generate size markers for the analysis of wild-type and mutant RNAs in the sample population. Thus each analysis involves two enzymatic nucleic acid molecules, two substrates and one unknown sample which is combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., HBV or HCV) is adequate to establish risk. If probes of comparable specific activity are used for both transcripts, then a qualitative comparison of RNA levels is adequate and will decrease the cost of the initial diagnosis. Higher mutant form to wild-type ratios are correlated with higher risk whether RNA levels are compared qualitatively or quantitatively.

Additional Uses

Potential usefulness of sequence-specific enzymatic nucleic acid molecules of the instant invention have many of the same applications for the study of RNA that DNA restriction endonucleases have for the study of DNA (Nathans et al., 1975 Ann. Rev. Biochem. 44:273). For example, the pattern of restriction fragments can be used to establish sequence relationships between two related RNAs, and large RNAs can be specifically cleaved to fragments of a size more useful for study. The ability to engineer sequence specificity of the enzymatic nucleic acid molecule is ideal for cleavage of RNAs of unknown sequence. Applicant describes the use of nucleic acid molecules to down-regulate gene expression of target genes in bacterial, microbial, fungal, viral, and eukaryotic systems including plant, or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably can be practiced in the absence of any element or elements, limitation or limitations that are not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

TABLE I
Characteristics of naturally occurring ribozymes

Group I Introns
Size: ˜150 to > 1000 nucleotides.
Requires a U in the target sequence immediately 5′ of the cleavage site.
Binds 4-6 nucleotides at the 5′-side of the cleavage site.
Reaction mechanism: aftack by the 3′-OH of guanosine to generate cleavage
products with 3′-OH and 5′-guanosine.
Additional protein cofactors required in some cases to help folding and
maintenance of the active structure.
Over 300 known members of this class. Found as an intervening sequence in
Tetrahymena thermophila rRNA, fungal mitochondria, chloroplasts, phage T4,
blue-green algae, and others.
Major structural features largely established through phylogenetic comparisons,
mutagenesis, and biochemical studies [ⁱ,ⁱⁱ].
Complete kinetic framework established for one ribozyme [ⁱⁱⁱ,^{iv},^{v},^{vi}].
Studies of ribozyme folding and substrate docking underway [^{vii},^{viii},^{ix}].
Chemical modification investigation of important residues well established [^{xxi}].
The small (4-6 nt) binding site may make this ribozyme too non-specific for
targeted RNA cleavage, however, the Tetrahymena group I intron has been used to repair
a “defective” β-galactosidase message by the ligation of new
β-galactosidase sequences onto the defective message [^{xii}]
RNAse P RNA (M1 RNA)
Size: ˜290 to 400 nucleotides.
RNA portion of a ubiquitous ribonucleoprotein enzyme.
Cleaves tRNA precursors to form mature tRNA [^{xiii}]
Reaction mechanism: possible attack by M²⁺-OH to generate cleavage products
with 3′-OH and 5′-phosphate.
RNAse P is found throughout the prokaryotes and eukaryotes. The RNA
subunit has been sequenced from bacteria, yeast, rodents, and primates.
Recruitment of endogenous RNAse P for therapeutic applications is possible
through hybridization of an External Guide Sequence (EGS) to the target RNA
[^{xiv},^{xv}]
Important phosphate and 2′ OH contacts recently identified [^{xvi},^{xvii}]
Group II Introns
Size: > 1000 nucleotides.
Trans cleavage of target RNAs recently demonstrated [^{xviii},^{xvix}].
Sequence requirements not fully determined.
Reaction mechanism: 2′-OH of an internal adenosine generates cleavage
products with 3′-OH and a “lariat” RNA containing a 3′-5′ and a 2′-5′ branch point.
Only natural ribozyme with demonstrated participation in DNA cleavage [^{xx},^{xxi}]
in addition to RNA cleavage and ligation.
Major structural features largely established through phylogenetic comparisons
[^{xxii}].
Important 2′ OH contacts beginning to be identified [^{xxiii}]
Kinetic framework under development [^{xxiv}]
Neurospora VS RNA
Size: ˜144 nucleotides.
Trans cleavage of hairpin target RNAs recently demonstrated [^{xxv}].
Sequence requirements not fully determined.
Reaction mechanism: attack by 2′-OH 5′ to the scissile bond to generate
cleavage products with 2′,3′-cyclic phosphate and 5′-OH ends.
Binding sites and structural requirements not fully determined.
Only 1 known member of this class. Found in Neurospora VS RNA.
Hammerhead Ribozyme
(see text for references)
Size: ˜13 to 40 nucleotides.
Requires the target sequence UH immediately 5′ of the cleavage site.
Binds a variable number nucleotides on both sides of the cleavage site.
Reaction mechanism: attack by 2′-OH 5′ to the scissile bond to generate
cleavage products with 2′,3′-cyclic phosphate and 5′-OH ends.
14 known members of this class. Found in a number of plant pathogens
(virusoids) that use RNA as the infectious agent.
Essential structural features largely defined, including 2 crystal structures
[^{xxvi},^{xxvii}]
Minimal ligation activity demonstrated (for engineering through in vitro
selection) [^{xxviii}]
Complete kinetic framework established for two or more ribozymes [^{xxix}]
Chemical modification investigation of important residues well established [^{xxx}].
Hairpin Ribozyme
Size: ˜50 nucleotides.
Requires the target sequence GUC immediately 3′of the cleavage site.
Binds 4-6 nucleotides at the 5′-side of the cleavage site and a variable number to
the 3′-side of the cleavage site.
Reaction mechanism: attack by 2′-OH 5′ to the scissile bond to generate
cleavage products with 2′,3′-cyclic phosphate and 5′-OH ends.
3 known members of this class. Found in three plant pathogen (satellite RNAs
of the tobacco ringspot virus, arabis mosaic virus and chicory yellow mottle
virus) which uses RNA as the infectious agent.
Essential structural features largely defined [^{xxxi},^{xxxii},^{xxiii},^{xxxiv}]
Ligation activity (in addition to cleavage activity) makes ribozyme amenable to
engineering through in vitro selection [^{xxxv}]
Complete kinetic framework established for one ribozyme [^{xxxvi}]
Chemical modification investigation of important residues begun [^{xxxvii},^{xxxviii}].
Hepatitis Delta Virus (HDV) Ribozyme
Size: ˜60 nucleotides.
Trans cleavage of target RNAs demonstrated [^{xxxix}].
Binding sites and structural requirements not fully determined, although no
sequences 5′ of cleavage site are required. Folded ribozyme contains a
pseudoknot structure [^{x1}].
Reaction mechanism: attack by 2′-OH 5′ to the scissile bond to generate
cleavage products with 2′,3′-cyclic phosphate and 5′-OH ends.
Only 2 known members of this class. Found in human HDV.
Circular form of HDV is active and shows increased nuclease stability [^{x1i}]
ⁱMichel, Francois; Westhof, Eric. Slippery substrates. Nat. Struct. Biol. (1994), 1(1), 5-7.
ⁱⁱLisacek, Frederique; Diaz, Yolande; Michel, Francois. Automatic identification of group I intron cores in genomic DNA sequences. J. Mol. Biol. (1994), 235 (4), 1206-17.
ⁱⁱⁱHerschlag, Daniel; Cech, Thomas R.. Catalysis of RNA cleavage by the Tetrahymena thermophila ribozyme. 1. Kinetic description of the reaction of an RNA substrate complementary to the active site. Biochemistry (1990), 29 (44), 10159-71.
^{iv}Herschlag, Daniel; Cech, Thomas R.. Catalysis of RNA cleavage by the Tetrahymena thermophila ribozyme. 2. Kinetic description of the reaction of an RNA substrate that forms a mismatch at the active site. Biochemistry (1990), 29 (44), 10172-80.
^{v}Knitt, Deborah S.; Herschlag, Daniel. pH Dependencies of the Tetrahymena Ribozyme Reveal an Unconventional Origin of an Apparent pKa. Biochemistry (1996), 35 (5), 1560-70.
^{vi}Bevilacqua, Philip C.; Sugimoto, Naoki; Turner, Douglas H.. A mechanistic framework for the second step of splicing catalyzed by the Tetrahymena ribozyme. Biochemistry (1996), 35 (2), 648-58.
^{vii}Li, Yi; Bevilacqua, Philip C.; Mathews, David; Turner, Douglas H.. Thermodynamic and activation parameters for binding of a pyrene-labeled substrate by the Tetrahymena ribozyme: docking is not diffusion-controlled and is driven by a favorable entropy change. Biochemistry (1995), 34 (44), 14394-9.
^{viii}Banerjee, Aloke Raj; Turner, Douglas H.. The time dependence of chemical modification reveals slow steps in the folding of a group I ribozyme. Biochemistry (1995), 34 (19), 6504-12.
^{ix}Zarrinkar, Patrick P.; Williamson, James R.. The P9.1-P9.2 peripheral extension helps guide folding of the Tetrahymena ribozyme. Nucleic Acids Res. (1996), 24 (5), 854-8.
^{x}Strobel, Scott A.; Cech, Thomas R.. Minor groove recognition of the conserved G.cntdot.U pair at the Tetrahymena ribozyme reaction site. Science (Washington, D. C.) (1995), 267 (5198), 675-9.
^{xi}Strobel, Scott A.; Cech, Thomas R.. Exocyclic Amine of the Conserved G.cntdot.U Pair at the Cleavage Site of the Tetrahymena Ribozyme Contributes to 5′-Splice Site Selection and Transition State Stabilization. Biochemistry (1996), 35 (4), 1201-11.
^{xii}Sullenger, Bruce A.; Cech, Thomas R.. Ribozyme-mediated repair of defective mRNA by targeted trans-splicing. Nature (London) (1994), 371 (6498), 619-22.
^{xiii}Robertson, H. D.; Altman, S.; Smith, J. D. J. Biol. Chem, 247 5243-5251 (1972).
^{xiv}Forster, Anthony C.; Altman, Sidney. External guide sequences for an RNA enzyme. Science (Washington, D. C., 1883-) (1990), 249 (4970), 783-6.
^{xv}Yuan, Y.; Hwang, E. S.; Altman, S. Targeted cleavage of mRNA by human RNase P. Proc. Natl. Acad. Sci. USA (1992) 89, 8006-10.
^{xvi}Harris, Michael E.; Pace, Norman R.. Identification of phosphates involved in catalysis by the ribozyme RNase P RNA. RNA (1995), 1 (2), 210-18.
^{xvii}Pan, Tao; Loria, Andrew; Zhong, Kun. Probing of tertiary interactions in RNA: 2′-hydroxyl-base contacts between the RNase P RNA and pre-tRNA. Proc. Natl. Acad. Sci. U. S. A. (1995), 92 (26), 12510-14.
^{xviii}Pyle, Anna Marie; Green, Justin B.. Building a Kinetic Framework for Group II Intron Ribozyme Activity: Quantitation of Interdomain Binding and Reaction Rate. Biochemistry (1994), 33 (9), 2716-25.
^{xix}Michels, William J. Jr.; Pyle, Anna Marie. Conversion of a Group II Intron into a New Multiple-Turnover Ribozyme that Selectively Cleaves Oligonucleotides: Elucidation of Reaction Mechanism and Structure/Function Relationships. Biochemistry (1995), 34 (9), 2965-77.
^{xx}Zimmerly, Steven; Guo, Huatao; Eskes, Robert; Yang, Jian; Perlman, Philip S.; Lambowitz, Alan M.. A group II intron RNA is a catalytic component of a DNA endonuclease involved in intron mobility. Cell (Cambridge, Mass.) (1995), 83 (4), 529-38.
^{xxi}Griffin, Edmund A., Jr.; Qin, Zhifeng; Michels, Williams J., Jr.; Pyle, Anna Marie. Group II intron ribozymes that cleave DNA and RNA linkages with similar efficiency, and lack contacts with substrate 2-hydroxyl groups. Chem. Biol. (1995), 2 (11), 761-70.
^{xxii}Michel, Francois; Ferat, Jean Luc. Structure and activities of group II introns. Annu. Rev. Biochem. (1995), 64, 435-61.
^{xxiii}Abramovitz, Dana L.; Friedman, Richard A.; Pyle, Anna Marie. Catalytic role of 2′-hydroxyl groups within a group II intron active site. Science (Washington, D. C.) (1996), 271 (5254), 1410-13.
^{xxiv}Daniels, Danette L.; Michels, William J., Jr.; Pyle, Anna Marie. Two competing pathways for self-splicing by group II introns: a quantitative analysis of in vitro reaction rates and products. J. Mol. Biol. (1996), 256 (1), 3149.
^{xxv}Guo, Hans C. T.; Collins, Richard A.. Efficient trans-cleavage of a stem-loop RNA substrate by a ribozyme derived from Neurospora VS RNA. EMBO J. (1995), 14 (2), 368-76.
^{xxvi}Scott, W. G., Finch, J. T., Aaron, K. The crystal structure of an all RNA hammerhead ribozyme: Aproposed mechanism for RNA catalytic cleavage. Cell, (1995), 81, 991-1002.
^{xxvii}McKay, Structure and function of the hammerhead ribozyme: an unfinished story. RNA, (1996), 2, 395-403.
^{xxviii}Long, D., Uhlenbeck, O., Hertel, K. Ligation with hammerhead ribozymes. U.S. Pat. No. 5,633,133.
^{xxix}Hertel, K. J., Herschlag, D., Uhlenbeck, O. A kinetic and thermodynamic framework for the hammerhead ribozyme reaction. Biochemistry, (1994) 33, 3374-3385.Beigehnan, L., et al, Chemical modifications of hammerhead ribozymes. J. Biol. Chem., (1995) 270, 25702-25708.
^{xxx}Beigelman, L., et al., Chemical modifications of hammerhead ribozymes. J. Biol. Chem., (1995) 270, 25702-25708.
^{xxxi}Hampel, Arnold; Tritz, Richard; Hicks, Margaret; Cruz, Philip. ‘Hairpin’ catalytic RNA model: evidence for helixes and sequence requirement for substrate RNA. Nucleic Acids Res. (1990), 18 (2), 299-304.
^{xxxii}Chowrira, Bliarat M.; Berzal-Herranz, Aifredo; Burke, John M.. Novel guanosine requirement for catalysis by the hairpin ribozyme. Nature (London) (1991), 354 (6351), 320-2.
^{xxxiii}Berzal-Herranz, Alfredo; Joseph, Simpson; Chowrira, Bharat M.; Butcher, Samuel E.; Burke, John M.. Essential nucleotide sequences and secondary structure elements of the hairpin ribozyme. EMBO J. (1993), 12 (6), 2567-73.
^{xxxiv}Joseph, Simpson; Berzal-Herranz, Aifredo; Chowrira, Bharat M.; Butcher, Samuel E.. Substrate selection rules for the hairpin ribozyme determined by in vitro selection, mutation, and analysis of mismatched substrates. Genes Dev. (1993), 7 (1), 130-8.
^{xxxv}Berzal-Herranz, Aifredo; Joseph, Simpson; Burke, John M.. In vitro selection of active hairpin ribozymes by sequential RNA-catalyzed cleavage and ligation reactions. Genes Dev. (1992), 6 (1), 129-34.
^{xxxvi}Hegg, Lisa A.; Fedor, Martha J.. Kinetics and Thermodynamics of Intermolecular Catalysis by Hairpin Ribozymes. Biochemistry (1995), 34 (48), 15813-28.
^{xxxvii}Grasby, Jane A.; Mersmann, Karin; Singh, Mohinder; Gait, Michael J.. Purine Functional Groups in Essential Residues of the Hairpin Ribozyme Required for Catalytic Cleavage of RNA. Biochemistry (1995), 34 (12), 4068-76.
^{xxxviii}Schmidt, Sabine; Beigelman, Leonid; Karpeisky, Alexander; Usman, Nassim; Sorensen, Ulrik S.; Gait, Michael J.. Base and sugar requirements for RNA cleavage of essential nucleoside residues in internal loop B of the hairpin ribozyme: implications for secondary structure. Nucleic Acids Res. (1996), 24 (4), 573-81.
^{xxxix}Perrotta, Anne T.; Been, Michael D.. Cleavage of oligoribonucleotides by a ribozyme derived from the hepatitis .delta. virus RNA sequence. Biochemistry (1992), 31 (1), 16-21.
^{x1}Perrotta, Anne T.; Been, Michael D.. A pseudoknot-like structure required for efficient self-cleavage of hepatitis delta virus RNA. Nature (London) (1991), 350 (6317), 434-6.
^{x1i}Puttaraju, M.; Perrotta, Anne T.; Been, Michael D.. A circular trans-acting hepatitis delta virus ribozyme. Nucleic Acids Res. (1993), 21 (18), 4253-8.

TABLE II

| | | | | | |
|---|---|---|---|---|---|
| | | | | Wait Time* | |
| Reagent | Equivalents | Amount | Wait Time* DNA | 2′-O-methyl | Wait Time*RNA |

A. 2.5 μmol Synthesis Cycle ABI 394 Instrument

| | | | | | | | | |
|---|---|---|---|---|---|---|---|---|
| Phosphoramidites | 6.5 | 163 | μL | 45 sec | 2.5 | min | 7.5 | min |
| S-Ethyl Tetrazole | 23.8 | 238 | μL | 45 sec | 2.5 | min | 7.5 | min |
| Acetic Anhydride | 100 | 233 | μL | 5 sec | 5 | sec | 5 | sec |
| N-Methyl | 186 | 233 | μL | 5 sec | 5 | sec | 5 | sec |
| Imidazole | | | | | | | | |
| TCA | 176 | 2.3 | mL | 21 sec | 21 | sec | 21 | sec |
| Iodine | 11.2 | 1.7 | mL | 45 sec | 45 | sec | 45 | sec |
| Beaucage | 12.9 | 645 | μL | 100 sec | 300 | sec | 300 | sec |

| | | | | | | |
|---|---|---|---|---|---|---|
| Acetonitrile | NA | 6.67 | mL | NA | NA | NA |

B. 0.2 μmol Synthesis Cycle ABI 394 Instrument

| | | | | | | | | |
|---|---|---|---|---|---|---|---|---|
| Phosphoramidites | 15 | 31 | μL | 45 sec | 233 | sec | 465 | sec |
| S-Ethyl Tetrazole | 38.7 | 31 | μL | 45 sec | 233 | min | 465 | sec |
| Acetic Anhydride | 655 | 124 | μL | 5 sec | 5 | sec | 5 | sec |
| N-Methyl | 1245 | 124 | μL | 5 sec | 5 | sec | 5 | sec |
| Imidazole | | | | | | | | |
| TCA | 700 | 732 | μL | 10 sec | 10 | sec | 10 | sec |
| Iodine | 20.6 | 244 | μL | 15 sec | 15 | sec | 15 | sec |
| Beaucage | 7.7 | 232 | μL | 100 sec | 300 | sec | 300 | sec |

| | | | | | | |
|---|---|---|---|---|---|---|
| Acetonitrile | NA | 2.64 | mL | NA | NA | NA |

| | | | | | |
|---|---|---|---|---|---|
| | Equivalents: | Amount: | | Wait Time* | |
| | DNA/2′-O- | DNA/2′-O- | Wait Time* | 2′-O- | Wait Time* |
| Reagent | methyl/Ribo | methyl/Ribo | DNA | methyl | Ribo |

C. 0.2 μmol Synthesis Cycle 96 well Instrument

| | | | | | | | |
|---|---|---|---|---|---|---|---|
| Phosphoramidites | 22/33/66 | 40/60/120 | μL | 60 sec | 180 | sec | 360 sec |
| S-Ethyl Tetrazole | 70/105/210 | 40/60/120 | μL | 60 sec | 180 | min | 360 sec |
| Acetic Anhydride | 265/265/265 | 50/50/50 | μL | 10 sec | 10 | sec | 10 sec |
| N-Methyl | 502/502/502 | 50/50/50 | μL | 10 sec | 10 | sec | 10 sec |
| Imidazole | | | | | | | |
| TCA | 238/475/475 | 250/500/500 | μL | 15 sec | 15 | sec | 15 sec |
| Iodine | 6.8/6.8/6.8 | 80/80/80 | μL | 30 sec | 30 | sec | 30 sec |
| Beaucage | 34/51/51 | 80/120/120 | | 100 sec | 200 | sec | 200 sec |

| | | | | | | |
|---|---|---|---|---|---|---|
| Acetonitrile | NA | 1150/1150/1150 | μL | NA | NA | NA |

*Wait time does not include contact time during delivery.

TABLE III
HBV Strains and Accession numbers

| Accession | |
|---|---|
| Number | NAME |
| AF100308.1 | AF100308 Hepatitis B virus strain 2-18, complete |
| AB026815.1 | AB026815 Hepatitis B virus DNA, complete genome, |
| AB033559.1 | AB033559 Hepatitis B virus DNA, complete genome, |
| AB033558.1 | AB033558 Hepatitis B virus DNA, complete genome, |
| AB033557.1 | AB033557 Hepatitis B virus DNA, complete genome, |
| AB033556.1 | AB033556 Hepatitis B virus DNA, complete genome, |
| AB033555.1 | AB033555 Hepatitis B virus DNA, complete genome, |
| AB033554.1 | AB033554 Hepatitis B virus DNA, complete genome, |
| AB033553.1 | AB033553 Hepatitis B virus DNA, complete genome, |
| AB033552.1 | AB033552 Hepatitis B virus DNA, complete genome, |
| AB033551.1 | AB033551 Hepatitis B virus DNA, complete genome, |
| AB033550.1 | AB033550 Hepatitis B virus DNA, complete genome |
| AF143308.1 | AF143308 Hepatitis B virus clone WB1254, complete |
| AF143307.1 | AF143307 Hepatitis B virus clone RM518, complete |
| AF143306.1 | AF143306 Hepatitis B virus clone RM517, complete |
| AF143305.1 | AF143305 Hepatitis B virus clone RM501, complete |
| AF143304.1 | AF143304 Hepatitis B virus clone HD319, complete |
| AF143303.1 | AF143303 Hepatitis B virus clone HD1406, complete |
| AF143302.1 | AF143302 Hepatitis B virus clone HD1402, complete |
| AF143301.1 | AF143301 Hepatitis B virus clone BW1903, complete |
| AF143300.1 | AF143300 Hepatitis B virus clone 7832-G4, complete |
| AF143299.1 | AF143299 Hepatitis B virus clone 7744-G9, complete |
| AF143298.1 | AF143298 Hepatitis B virus clone 7720-G8, complete |
| AB026814.1 | AB026814 Hepatitis B virus DNA, complete genome, |
| AB026813.1 | AB026813 Hepatitis B virus DNA, complete genome, |
| AB026812.1 | AB026812 Hepatitis B virus DNA, complete genome, |
| AB026811.1 | AB026811 Hepatitis B virus DNA, complete genome, |
| AJ131956.1 | HBV131956 Hepatitis B virus complete genome, |
| AF151735.1 | AF151735 Hepatitis B virus, complete genome |
| AF090842.1 | AF090842 Hepatitis B virus strain G5.27295, complete |
| AF090841.1 | AF090841 Hepatitis B virus strain G4.27241, complete |
| AF090840.1 | AF090840 Hepatitis B virus strain G3.27270, complete |
| AF090839.1 | AF090839 Hepatitis B virus strain G2.27246, complete |
| AF090838.1 | AF090838 Hepatitis B virus strain P1.27239, complete |
| Y18858.1 | HBV18858 Hepatitis B virus complete genome, isolate |
| Y18857.1 | HBV18857 Hepatitis B virus complete genome, isolate |
| D12980.1 | HPBCG Hepatitis B virus subtype adr (SRADR) DNA, |
| Y18856.1 | HBV18856 Hepatitis B virus complete genome, isolate |
| Y18855.1 | HBV18855 Hepatitis B virus complete genome, isolate |
| AJ131133.1 | HBV131133 Hepatitis B virus, complete genome, strain |
| X80925.1 | HBVP6PCXX Hepatitis B virus (patient 6) complete |
| X80926.1 | HBVP5PCXX Hepatitis B virus (patient 5) complete |
| X80924.1 | HBVP4PCXX Hepatitis B virus (patient 4) complete |
| AF100309.1 | Hepatitis B virus strain 56, complete genome |
| AF068756.1 | AF068756 Hepatitis B virus, complete genome |
| AF043593.1 | AF043593 Hepatitis B virus isolate 6/89, complete |
| Y07587.1 | HBVAYWGEN Hepatitis B virus, complete genome |
| D28880.1 | D28880 Hepatitis B virus DNA, complete genome, strain |
| X98076.1 | HBVDEFVP3 Hepatitis B virus complete genome with |
| X98075.1 | HBVDEFVP2 Hepatitis B virus complete genome with |
| X98074.1 | HBVDEFVP1 Hepatitis B virus complete genome with |
| X98077.1 | HBVCGWITY Hepatitis B virus complete genome, |
| | wild type |
| X98072.1 | HBVCGINSC Hepatitis B virus complete genome with |
| X98073.1 | HBVCGINCX Hepatitis B virus complete genome with |
| U95551.1 | U95551 Hepatitis B virus subtype ayw, complete genome |
| D23684.1 | HPBC6T588 Hepatitis B virus (C6-TKB588) complete |
| | genome |
| D23683.1 | HPBC5HKO2 Hepatitis B virus (C5-HBVKO2) complete |
| | genome |
| D23682.1 | HPBB5HKO1 Hepatitis B virus (B5-HBVKO1) complete |
| | genome |
| D23681.1 | HPBC4HST2 Hepatitis B virus (C4-HBVST2) complete |
| | genome |
| D23680.1 | HPBB4HST1 Hepatitis B virus (B4-HBVST1) complete |
| | genome |
| D00331.1 | HPBADW3 Hepatitis B virus genome, complete genome |
| D00330.1 | HPBADW2 Hepatitis B virus genome, complete genome |
| D50489.1 | HPBA11A Hepatitis B virus DNA, complete genome |
| D23679.1 | HPBA3HMS2 Hepatitis B virus (A3-HBVMS2) complete |
| | genome |
| D23678.1 | HPBA2HYS2 Hepatitis B virus (A2-HBVYS2) complete |
| | genome |
| D23677.1 | HPBA1HKK2 Hepatitis B virus (A1-HBVKK2) complete |
| | genome |
| D16665.1 | HPBADRM Hepatitis B virus DNA, complete genome |
| D00329.1 | HPBADW1 Hepatitis B virus (HBV) genome, complete |
| | genome |
| X97851.1 | HBVP6CSX Hepatitis B virus (patient 6) complete genome |
| X97850.1 | HBVP4CSX Hepatitis B virus (patient 4) complete genome |
| X97849.1 | HBVP3CSX Hepatitis B virus (patient 3) complete genome |
| X97848.1 | HBVP2CSX Hepatitis B virus (patient 2) complete genome |
| X51970.1 | HVHEPB Hepatitis B virus (HBV 991) complete genome |
| M38636.1 | HPBCGADR Hepatitis B virus, subtype adr, complete |
| | genome |
| X59795.1 | HBVAYWMCG Hepatitis B virus (ayw subtype mutant) |
| M38454.1 | HPBADR1CG Hepatitis B virus, complete genome |
| M32138.1 | HPBHBVAA Hepatitis B virus variant HBV-alpha1, |
| | complete |
| J02203.1 | HPBAYW Human hepatitis B virus (subtype ayw), |
| | complete |
| M12906.1 | HPBADRA Hepatitis B virus subtype adr, complete |
| | genome |
| M54923.1 | HPBADWZ Hepatitis B virus (subtype adw), complete |
| | genome |
| L27106.1 | HPBMUT Hepatitis B virus mutant complete genome |

TABLE IV

HBV Substrate Sequence

| NT Position* | SUBSTRATE | SEQ ID |
|---|---|---|
| 82 | CUAUCGUCCCCUUCUUCAUC | 1. |
| 101 | CUACCGUUCCGGCC | 2. |
| 159 | CUUCUCAUCU | 3. |
| 184 | CUUCCCUUCACCAC | 4. |
| 269 | GACUCUCAGAAUGUCAACGAC | 5. |
| 381 | CUGUAGGCAUAAAUGGUCUG | 6. |
| 401 | GUUCACCAGCACCAUGCAACUUUUU | 7. |
| 424 | UUUCACGUCUGCCUAAUCAUC | 8. |
| 524 | AUUUGGAGCUUC | 9. |
| 562 | CUGACUUCUUUCCUUCUAUUC | 10. |
| 649 | CUCACCAUACCGCACUCA | 11. |
| 667 | GGCAAGCUAUUCUGUG | 12. |
| 717 | GGAAGUAAUUUGGAAGAC | 13. |
| 758 | CAGCUAUGUCAAUGUUAA | 14. |
| 783 | CUAAAAUCGGCCUAAAAUCAGAC | 15. |
| 812 | CAUUUCCUGUCUCACUUUUGGAAGAG | 16. |
| 887 | UCCUGCUUACAGAC | 17. |
| 922 | CAACACUUCCGGAAACUACUGUUGUUAG | 18. |
| 989 | CUCGCCUCGCAGACGAAGGUCUC | 19. |
| 1009 | CAAUCGCCGCGUCGCAGAAG | 20. |
| 1031 | AUCUCAAUCUCGGGAAUCUCAA | 21. |
| 1052 | AUGUUAGUAUCCCUUGGACUC | 22. |
| 1072 | CAUAAGGUGGGAAACUUUACUG | 23. |
| 1109 | CUGUACCUAUUCUUUAAAUCC | 24. |
| 1127 | CUGAGUGGCAAACUCCC | 25. |
| 1271 | CCAAAUAUCUGCCCUUGGACAA | 26. |
| 1297 | AUUAAACCAUAUUAUCCUGAACA | 27. |
| 1319 | AUGCAGUUAAUCAUUACUUCAAAACUA | 28. |
| 1340 | AAACUAGGCAUEJA | 29. |
| 1370 | AGGCGGGCAUUCUAUAUAAGAGAG | 30. |
| 1393 | GAAACUACGCGCAGCGCCUCAUUUUGU | 31. |
| 1412 | CAUUUUGUGGGUCACCAUA | 32. |
| 1441 | CAAGAGCUACAGCAUGGG | 33. |

LOCUS HPBADR1CG 3221 bp DNA circular VRL 06 MAR. 1995
DEFINITION Hepatitis B virus, complete genome.
ACCESSION M38454
*The nucleotide number referred to in that table is the position of the 5′ end of the oligo in this sequence.

TABLE V

HUMAN HBV HAMMERHEAD RIBOZYME AND TARGET SEQUENCE

| | | | | |
|---|---|---|---|---|
| | | Seq | | Seq |
| Pos | Substrate | ID | Hammerhead | ID |

| | | | | |
|---|---|---|---|---|
| 13 | CCACCACU U UCCACCAA | 34 | UUGGUGGA CUGAUGAG GCCGUUAGGC CGAA AGUGGUGG | 7434 |
| 14 | CACCACUU U CCACCAAA | 34 | UUUGGUGG CUGAUGAG GCCGUUAGGC CGAA AAGUGGUG | 7435 |
| 15 | ACCACUUU C CACCAAAC | 36 | GUUUGGUG CUGAUGAG GCCGUUAGGC CGAA AAAGUGGU | 7436 |
| 25 | ACCAAACU C UUCAAGAU | 37 | AUCUUGAA CUGAUGAG GCCGUUAGGC CGAA AGUUUGGU | 7437 |
| 27 | CAAACUCU U CAAGAUCC | 38 | GGAUCUUG CUGAUGAG GCCGUUAGGC CGAA AGAGUUUG | 7438 |
| 28 | AAACUCUU C AAGAUCCC | 39 | GGGAUCUU CUGAUGAG GCCGUUAGGC CGAA AAGAGUUU | 7439 |
| 34 | UUCAAGAU C CCAGAGUC | 40 | GACUCUGG CUGAUGAG GCCGUUAGGC CGAA AUCUUGAA | 7440 |
| 42 | CCCAGAGU C AGGGCCCU | 41 | AGGGCCCU CUGAUGAG GCCGUUAGGC CGAA ACUCUGGG | 7441 |
| 53 | GGCCCUGU A CUUUCCUG | 42 | CAGGAAAG CUGAUGAG GCCGUUAGGC CGAA ACAGGGCC | 7442 |
| 56 | CCUGUACU U UCCUGCUG | 43 | CAGCAGGA CUGAUGAG GCCGUUAGGC CGAA AGUACAGG | 7443 |
| 57 | CUGUACUU U CCUGCUGG | 44 | CCAGCAGG CUGAUGAG GCCGUUAGGC CGAA AAGUACAG | 7444 |
| 58 | UGUACUUU C CUGCUGGU | 45 | ACCAGCAG CUGAUGAG GCCGUUAGGC CGAA AAAGUACA | 7445 |
| 71 | UGGUGGCU C CAGUUCAG | 46 | CUGAACUG CUGAUGAG GCCGUUAGGC CGAA AGCCACCA | 7446 |
| 76 | GCUCCAGU U CAGGAACA | 47 | UGUUCCUG CUGAUGAG GCCGUUAGGC CGAA ACUGGAGC | 7447 |
| 77 | CUCCAGUU C AGGAACAG | 48 | CUGUUCCU CUGAUGAG GCCGUUAGGC CGAA AACUGGAG | 7448 |
| 97 | GCCCUGCU C AGAAUACU | 49 | AGUAUUCU CUGAUGAG GCCGUUAGGC CGAA AGCAGGGC | 7449 |
| 103 | CUCAGAAU A CUGUCUCU | 50 | AGAGACAG CUGAUGAG GCCGUUAGGC CGAA AUUCUGAG | 7450 |
| 108 | AAUACUGU C UCUGCCAU | 51 | AUGGCAGA CUGAUGAG GCCGUUAGGC CGAA ACAGUAUU | 7451 |
| 110 | UACUGUCU C UGCCAUAU | 52 | AUAUGGCA CUGAUGAG GCCGUUAGGC CGAA AGACAGUA | 7452 |
| 117 | UCUGCCAU A UCGUCAAU | 53 | AUUGACGA CUGAUGAG GCCGUUAGGC CGAA AUGGCAGA | 7453 |
| 119 | UGCCAUAU C GUCAAUCU | 54 | AGAUUGAC CUGAUGAG GCCGUUAGGC CGAA AUAUGGCA | 7454 |
| 122 | CAUAUCGU C AAUCUUAU | 55 | AUAAGAUU CUGAUGAG GCCGUUAGGC CGAA ACGAUAUG | 7455 |
| 126 | UCGUCAAU C UUAUCGAA | 56 | UUCGAUAA CUGAUGAG GCCGUUAGGC CGAA AUUGACGA | 7456 |
| 128 | GUCAAUCU U AUCGAAGA | 57 | UCUUCGAU CUGAUGAG GCCGUUAGGC CGAA AGAUUGAC | 7457 |
| 129 | UCAAUCUU A UCGAAGAC | 58 | GUCUUCGA CUGAUGAG GCCGUUAGGC CGAA AAGAUUGA | 7458 |
| 131 | AAUCUUAU C GAAGACUG | 59 | CAGUCUUC CUGAUGAG GCCGUUAGGC CGAA AUAAGAUU | 7459 |
| 150 | GACCCUGU A CCGAACAU | 60 | AUGUUCGG CUGAUGAG GCCGUUAGGC CGAA ACAGGGUC | 7460 |
| 168 | GAGAACAU C GCAUCAGG | 61 | CCUGAUGC CUGAUGAG GCCGUUAGGC CGAA AUGUUCUC | 7461 |
| 173 | CAUCGCAU C AGGACUCC | 62 | GGAGUCCU CUGAUGAG GCCGUUAGGC CGAA AUGCGAUG | 7462 |
| 180 | UCAGGACU C CUAGGACC | 63 | GGUCCUAG CUGAUGAG GCCGUUAGGC CGAA AGUCCUGA | 7463 |
| 183 | GGACUCCU A GGACCCCU | 64 | AGGGGUCC CUGAUGAG GCCGUUAGGC CGAA AGGAGUCC | 7464 |
| 195 | CCCCUGCU C GUGUUACA | 65 | UGUAACAC CUGAUGAG GCCGUUAGGC CGAA AGCAGGGG | 7465 |
| 200 | GCUCGUGU U ACAGGCGG | 66 | CCGCCUGU CUGAUGAG GCCGUUAGGC CGAA ACACGAGC | 7466 |
| 201 | CUCGUGUU A CAGGCGGG | 67 | CCCGCCUG CUGAUGAG GCCGUUAGGC CGAA AACACGAG | 7467 |
| 212 | GGCGGGGU U UUUCUUGU | 68 | ACAAGAAA CUGAUGAG GCCGUUAGGC CGAA ACCCCGCC | 7468 |
| 213 | GCGGGGUU U UUCUUGUU | 69 | AACAAGAA CUGAUGAG GCCGUUAGGC CGAA AACCCCGC | 7469 |
| 214 | CGGGGUUU U UCUUGUUG | 70 | CAACAAGA CUGAUGAG GCCGUUAGGC CGAA AAACCCCG | 7470 |
| 215 | GGGGUUUU U CUUGUUGA | 71 | UCAACAAG CUGAUGAG GCCGUUAGGC CGAA AAAACCCC | 7471 |
| 216 | GGGUUUUU C UUGUUGAC | 72 | GUCAACAA CUGAUGAG GCCGUUAGGC CGAA AAAAACCC | 7472 |
| 218 | GUUUUUCU U GUUGACAA | 73 | UUGUCAAC CUGAUGAG GCCGUUAGGC CGAA AGAAAAAC | 7473 |
| 221 | UUUCUUGU U GACAAAAA | 74 | UUUUUGUC CUGAUGAG GCCGUUAGGC CGAA ACAAGAAA | 7474 |
| 231 | ACAAAAAU C CUCACAAU | 75 | AUUGUGAG CUGAUGAG GCCGUUAGGC CGAA AUUUUUGU | 7475 |
| 234 | AAAAUCCU C ACAAUACC | 76 | GGUAUUGU CUGAUGAG GCCGUUAGGC CGAA AGGAUUUU | 7476 |
| 240 | CUCACAAU A CCACAGAG | 77 | CUCUGUGG CUGAUGAG GCCGUUAGGC CGAA AUUGUGAG | 7477 |
| 250 | CACAGAGU C UAGACUCG | 78 | CGAGUCUA CUGAUGAG GCCGUUAGGC CGAA ACUCUGUG | 7478 |
| 252 | CAGAGUCU A GACUCGUG | 79 | CACGAGUC CUGAUGAG GCCGUUAGGC CGAA AGACUCUG | 7479 |
| 257 | UCUAGACU C GUGGUGGA | 80 | UCCACCAC CUGAUGAG GCCGUUAGGC CGAA AGUCUAGA | 7480 |
| 268 | GGUGGACU U CUCUCAAU | 81 | AUUGAGAG CUGAUGAG GCCGUUAGGC CGAA AGUCCACC | 7481 |
| 269 | GUGGACUU C UCUCAAUU | 82 | AAUUGAGA CUGAUGAG GCCGUUAGGC CGAA AAGUCCAC | 7482 |
| 271 | GGACUUCU C UCAAUUUU | 83 | AAAAUUGA CUGAUGAG GCCGUUAGGC CGAA AGAAGUCC | 7483 |
| 273 | ACUUCUCU C AAUUUUCU | 84 | AGAAAAUU CUGAUGAG GCCGUUAGGC CGAA AGAGAAGU | 7484 |
| 277 | CUCUCAAU U UUCUAGGG | 85 | CCCUAGAA CUGAUGAG GCCGUUAGGC CGAA AUUGAGAG | 7485 |
| 278 | UCUCAAUU U UCUAGGGG | 86 | CCCCUAGA CUGAUGAG GCCGUUAGGC CGAA AAUUGAGA | 7486 |
| 279 | CUCAAUUU U CUAGGGGG | 87 | CCCCCUAG CUGAUGAG GCCGUUAGGC CGAA AAAUUGAG | 7487 |
| 280 | UCAAUUUU C UAGGGGGA | 88 | UCCCCCUA CUGAUGAG GCCGUUAGGC CGAA AAAAUUGA | 7488 |
| 282 | AAUUUUCU A GGGGGAAC | 89 | GUUCCCCC CUGAUGAG GCCGUUAGGC CGAA AGAAAAUU | 7489 |
| 301 | CCGUGUGU C UUGGCCAA | 90 | UUGGCCAA CUGAUGAG GCCGUUAGGC CGAA ACACACGG | 7490 |
| 303 | GUGUGUCU U GGCCAAAA | 91 | UUUUGGCC CUGAUGAG GCCGUUAGGC CGAA AGACACAC | 7491 |
| 313 | GCCAAAAU U CGCAGUCC | 92 | GGACUGCG CUGAUGAG GCCGUUAGGC CGAA AUUUUGGC | 7492 |
| 314 | CCAAAAUU C GCAGUCCC | 93 | GGGACUGC CUGAUGAG GCCGUUAGGC CGAA AAUUUUGG | 7493 |
| 320 | UUCGCAGU C CCAAAUCU | 94 | AGAUUUGG CUGAUGAG GCCGUUAGGC CGAA ACUGCGAA | 7494 |
| 327 | UCCCAAAU C UCCAGUCA | 95 | UGACUGGA CUGAUGAG GCCGUUAGGC CGAA AUUUGGGA | 7495 |
| 329 | CCAAAUCU C CAGUCACU | 96 | AGUGACUG CUGAUGAG GCCGUUAGGC CGAA AGAUUUGG | 7496 |
| 334 | UCUCCAGU C ACUCACCA | 97 | UGGUGAGU CUGAUGAG GCCGUUAGGC CGAA ACUGGAGA | 7497 |
| 338 | CAGUCACU C ACCAACCU | 98 | AGGUUGGU CUGAUGAG GCCGUUAGGC CGAA AGUGACUG | 7498 |
| 349 | CAACCUGU U GUCCUCCA | 99 | UGGAGGAC CUGAUGAG GCCGUUAGGC CGAA ACAGGUUG | 7499 |
| 352 | CCUGUUGU C CUCCAAUU | 100 | AAUUGGAG CUGAUGAG GCCGUUAGGC CGAA ACAACAGG | 7500 |
| 355 | GUUGUCCU C CAAUUUGU | 101 | ACAAAUUG CUGAUGAG GCCGUUAGGC CGAA AGGACAAC | 7501 |
| 360 | CCUCCAAU U UGUCCUGG | 102 | CCAGGACA CUGAUGAG GCCGUUAGGC CGAA AUUGGAGG | 7502 |
| 361 | CUCCAAUU U GUCCUGGU | 103 | ACCAGGAC CUGAUGAG GCCGUUAGGC CGAA AAUUGGAG | 7503 |
| 364 | CAAUUUGU C CUGGUUAU | 104 | AUAACCAG CUGAUGAG GCCGUUAGGC CGAA ACAAAUUG | 7504 |
| 370 | GUCCUGGU U AUCGCUGG | 105 | CCAGCGAU CUGAUGAG GCCGUUAGGC CGAA ACCAGGAC | 7505 |
| 371 | UCCUGGUU A UCGCUGGA | 106 | UCCAGCGA CUGAUGAG GCCGUUAGGC CGAA AACCAGGA | 7506 |
| 373 | CUGGUUAU C GCUGGAUG | 107 | CAUCCAGC CUGAUGAG GCCGUUAGGC CGAA AUAACCAG | 7507 |
| 385 | GGAUGUGU C UGCGGCGU | 108 | ACGCCGCA CUGAUGAG GCCGUUAGGC CGAA ACACAUCC | 7508 |
| 394 | UGCGGCGU U UUAUCAUC | 109 | GAUGAUAA CUGAUGAG GCCGUUAGGC CGAA ACGCCGCA | 7509 |
| 395 | GCGGCGUU U UAUCAUCU | 110 | AGAUGAUA CUGAUGAG GCCGUUAGGC CGAA AACGCCGC | 7510 |
| 396 | CGGCGUUU U AUCAUCUU | 111 | AAGAUGAU CUGAUGAG GCCGUUAGGC CGAA AAACGCCG | 7511 |
| 397 | GGCGUUUU A UCAUCUUC | 112 | GAAGAUGA CUGAUGAG GCCGUUAGGC CGAA AAAACGCC | 7512 |
| 399 | CGUUUUAU C AUCUUCCU | 113 | AGGAAGAU CUGAUGAG GCCGUUAGGC CGAA AUAAAACG | 7513 |
| 402 | UUUAUCAU C UUCCUCUG | 114 | CAGAGGAA CUGAUGAG GCCGUUAGGC CGAA AUGAUAAA | 7514 |
| 404 | UAUCAUCU U CCUCUGCA | 115 | UGCAGAGG CUGAUGAG GCCGUUAGGC CGAA AGAUGAUA | 7515 |
| 405 | AUCAUCUU C CUCUGCAU | 116 | AUGCAGAG CUGAUGAG GCCGUUAGGC CGAA AAGAUGAU | 7516 |
| 408 | AUCUUCCU C UGCAUCCU | 117 | AGGAUGCA CUGAUGAG GCCGUUAGGC CGAA AGGAAGAU | 7517 |
| 414 | CUCUGCAU C CUGCUGCU | 118 | AGCAGCAG CUGAUGAG GCCGUUAGGC CGAA AUGCAGAG | 7518 |
| 423 | CUGCUGCU A UGCCUCAU | 119 | AUGAGGCA CUGAUGAG GCCGUUAGGC CGAA AGCAGCAG | 7519 |
| 429 | CUAUGCCU C AUCUUCUU | 120 | AAGAAGAU CUGAUGAG GCCGUUAGGC CGAA AGGCAUAG | 7520 |
| 432 | UGCCUCAU C UUCUUGUU | 121 | AACAAGAA CUGAUGAG GCCGUUAGGC CGAA AUGAGGCA | 7521 |
| 434 | CCUCAUCU U CUUGUUGG | 122 | CCAACAAG CUGAUGAG GCCGUUAGGC CGAA AGAUGAGG | 7522 |
| 435 | CUCAUCUU C UUGUUGGU | 123 | ACCAACAA CUGAUGAG GCCGUUAGGC CGAA AAGAUGAG | 7523 |
| 437 | CAUCUUCU U GUUGGUUC | 124 | GAACCAAC CUGAUGAG GCCGUUAGGC CGAA AGAAGAUG | 7524 |
| 440 | CUUCUUGU U GGUUCUUC | 125 | GAAGAACC CUGAUGAG GCCGUUAGGC CGAA ACAAGAAG | 7525 |
| 444 | UUGUUGGU U CUUCUGGA | 126 | UCCAGAAG CUGAUGAG GCCGUUAGGC CGAA ACCAACAA | 7526 |
| 445 | UGUUGGUU C UUCUGGAC | 127 | GUCCAGAA CUGAUGAG GCCGUUAGGC CGAA AACCAACA | 7527 |
| 447 | UUGGUUCU U CUGGACUA | 128 | UAGUCCAG CUGAUGAG GCCGUUAGGC CGAA AGAACCAA | 7528 |
| 448 | UGGUUCUU C UGGACUAU | 129 | AUAGUCCA CUGAUGAG GCCGUUAGGC CGAA AAGAACCA | 7529 |
| 455 | UCUGGACU A UCAAGGUA | 130 | UACCUUGA CUGAUGAG GCCGUUAGGC CGAA AGUCCAGA | 7530 |
| 457 | UGGACUAU C AAGGUAUG | 131 | CAUACCUU CUGAUGAG GCCGUUAGGC CGAA AUAGUCCA | 7531 |
| 463 | AUCAAGGU A UGUUGCCC | 132 | GGGCAACA CUGAUGAG GCCGUUAGGC CGAA ACCUUGAU | 7532 |
| 467 | AGGUAUGU U GCCCGUUU | 133 | AAACGGGC CUGAUGAG GCCGUUAGGC CGAA ACAUACCU | 7533 |
| 474 | UUGCCCGU U UGUCCUCU | 134 | AGAGGACA CUGAUGAG GCCGUUAGGC CGAA ACGGGCAA | 7534 |
| 475 | UGCCCGUU U GUCCUCUA | 135 | UAGAGGAC CUGAUGAG GCCGUUAGGC CGAA AACGGGCA | 7535 |
| 478 | CCGUUUGU C CUCUAAUU | 136 | AAUUAGAG CUGAUGAG GCCGUUAGGC CGAA ACAAACGG | 7536 |
| 481 | UUUGUCCU C UAAUUCCA | 137 | UGGAAUUA CUGAUGAG GCCGUUAGGC CGAA AGGACAAA | 7537 |
| 483 | UGUCCUCU A AUUCCAGG | 138 | CCUGGAAU CUGAUGAG GCCGUUAGGC CGAA AGAGGACA | 7538 |
| 486 | CCUCUAAU U CCAGGAUC | 139 | GAUCCUGG CUGAUGAG GCCGUUAGGC CGAA AUUAGAGG | 7539 |
| 487 | CUCUAAUU C CAGGAUCA | 140 | UGAUCCUG CUGAUGAG GCCGUUAGGC CGAA AAUUAGAG | 7540 |
| 494 | UCCAGGAU C AUCAACAA | 141 | UUGUUGAU CUGAUGAG GCCGUUAGGC CGAA AUCCUGGA | 7541 |
| 497 | AGGAUCAU C AACAACCA | 142 | UGGUUGUU CUGAUGAG GCCGUUAGGC CGAA AUGAUCCU | 7542 |
| 535 | GCACAACU C CUGCUCAA | 143 | UUGAGCAG CUGAUGAG GCCGUUAGGC CGAA AGUUGUGC | 7543 |
| 541 | CUCCUGCU C AAGGAACC | 144 | GGUUCCUU CUGAUGAG GCCGUUAGGC CGAA AGCAGGAG | 7544 |
| 551 | AGGAACCU C UAUGUUUC | 145 | GAAACAUA CUGAUGAG GCCGUUAGGC CGAA AGGUUCCU | 7545 |
| 553 | GAACCUCU A UGUUUCCC | 146 | GGGAAACA CUGAUGAG GCCGUUAGGC CGAA AGAGGUUC | 7546 |
| 557 | CUCUAUGU U UCCCUCAU | 147 | AUGAGGGA CUGAUGAG GCCGUUAGGC CGAA ACAUAGAG | 7547 |
| 558 | UCUAUGUU U CCCUCAUG | 148 | CAUGAGGG CUGAUGAG GCCGUUAGGC CGAA AACAUAGA | 7548 |
| 559 | CUAUGUUU C CCUCAUGU | 149 | ACAUGAGG CUGAUGAG GCCGUUAGGC CGAA AAACAUAG | 7549 |
| 563 | GUUUCCCU C AUGUUGCU | 150 | AGCAACAU CUGAUGAG GCCGUUAGGC CGAA AGGGAAAC | 7550 |
| 568 | CCUCAUGU U GCUGUACA | 151 | UGUACAGC CUGAUGAG GCCGUUAGGC CGAA ACAUGAGG | 7551 |
| 574 | GUUGCUGU A CAAAACCU | 152 | AGGUUUUG CUGAUGAG GCCGUUAGGC CGAA ACAGCAAC | 7552 |
| 583 | CAAAACCU A CGGACGGA | 153 | UCCGUCCG CUGAUGAG GCCGUUAGGC CGAA AGGUUUUG | 7553 |
| 604 | GCACCUGU A UUCCCAUC | 154 | GAUGGGAA CUGAUGAG GCCGUUAGGC CGAA ACAGGUGC | 7554 |
| 606 | ACCUGUAU U CCCAUCCC | 155 | GGGAUGGG CUGAUGAG GCCGUUAGGC CGAA AUACAGGU | 7555 |
| 607 | CCUGUAUU C CCAUCCCA | 156 | UGGGAUGG CUGAUGAG GCCGUUAGGC CGAA AAUACAGG | 7556 |
| 612 | AUUCCCAU C CCAUCAUC | 157 | GAUGAUGG CUGAUGAG GCCGUUAGGC CGAA AUGGGAAU | 7557 |
| 617 | CAUCCCAU C AUCUUGGG | 158 | CCCAAGAU CUGAUGAG GCCGUUAGGC CGAA AUGGGAUG | 7558 |
| 620 | CCCAUCAU C UUGGGCUU | 159 | AAGCCCAA CUGAUGAG GCCGUUAGGC CGAA AUGAUGGG | 7559 |
| 622 | CAUCAUCU U GGGCUUUC | 160 | GAAAGCCC CUGAUGAG GCCGUUAGGC CGAA AGAUGAUG | 7560 |
| 628 | CUUGGGCU U UCGCAAAA | 161 | UUUUGCGA CUGAUGAG GCCGUUAGGC CGAA AGCCCAAG | 7561 |
| 629 | UUGGGCUU U CGCAAAAU | 162 | AUUUUGCG CUGAUGAG GCCGUUAGGC CGAA AAGCCCAA | 7562 |
| 630 | UGGGCUUU C GCAAAAUA | 163 | UAUUUUGC CUGAUGAG GCCGUUAGGC CGAA AAAGCCCA | 7563 |
| 638 | CGCAAAAU A CCUAUGGG | 164 | CCCAUAGG CUGAUGAG GCCGUUAGGC CGAA AUUUUGCG | 7564 |
| 642 | AAAUACCU A UGGGAGUG | 165 | CACUCCCA CUGAUGAG GCCGUUAGGC CGAA AGGUAUUU | 7565 |
| 656 | GUGGGCCU C AGUCCGUU | 166 | AACGGACU CUGAUGAG GCCGUUAGGC CGAA AGGCCCAC | 7566 |
| 660 | GCCUCAGU C CGUUUCUC | 167 | GAGAAACG CUGAUGAG GCCGUUAGGC CGAA ACUGAGGC | 7567 |
| 664 | CAGUCCGU U UCUCUUGG | 168 | CCAAGAGA CUGAUGAG GCCGUUAGGC CGAA ACGGACUG | 7568 |
| 665 | AGUCCGUU U CUCUUGGC | 169 | GCCAAGAG CUGAUGAG GCCGUUAGGC CGAA AACGGACU | 7569 |
| 666 | GUCCGUUU C UCUUGGCU | 170 | AGCCAAGA CUGAUGAG GCCGUUAGGC CGAA AAACGGAC | 7570 |
| 668 | CCGUUUCU C UUGGCUCA | 171 | UGAGCCAA CUGAUGAG GCCGUUAGGC CGAA AGAAACGG | 7571 |
| 670 | GUUUCUCU U GGCUCAGU | 172 | ACUGAGCC CUGAUGAG GCCGUUAGGC CGAA AGAGAAAC | 7572 |
| 675 | UCUUGGCU C AGUUUACU | 173 | AGUAAACU CUGAUGAG GCCGUUAGGC CGAA AGCCAAGA | 7573 |
| 679 | GGCUCAGU U UACUAGUG | 174 | CACUAGUA CUGAUGAG GCCGUUAGGC CGAA ACUGAGCC | 7574 |
| 680 | GCUCAGUU U ACUAGUGC | 175 | GCACUAGU CUGAUGAG GCCGUUAGGC CGAA AACUGAGC | 7575 |
| 681 | CUCAGUUU A CUAGUGCC | 176 | GGCACUAG CUGAUGAG GCCGUUAGGC CGAA AAACUGAG | 7576 |
| 684 | AGUUUACU A GUGCCAUU | 177 | AAUGGCAC CUGAUGAG GCCGUUAGGC CGAA AGUAAACU | 7577 |
| 692 | AGUGCCAU U UGUUCAGU | 178 | ACUGAACA CUGAUGAG GCCGUUAGGC CGAA AUGGCACU | 7578 |
| 693 | GUGCCAUU U GUUCAGUG | 179 | CACUGAAC CUGAUGAG GCCGUUAGGC CGAA AAUGGCAC | 7579 |
| 696 | CCAUUUGU U CAGUGGUU | 180 | AACCACUG CUGAUGAG GCCGUUAGGC CGAA ACAAAUGG | 7580 |
| 697 | CAUUUGUU C AGUGGUUC | 181 | GAACCACU CUGAUGAG GCCGUUAGGC CGAA AACAAAUG | 7581 |
| 704 | UCAGUGGU U CGUAGGGC | 182 | GCCCUACG CUGAUGAG GCCGUUAGGC CGAA ACCACUGA | 7582 |
| 705 | CAGUGGUU C GUAGGGCU | 183 | AGCCCUAC CUGAUGAG GCCGUUAGGC CGAA AACCACUG | 7583 |
| 708 | UGGUUCGU A GGGCUUUC | 184 | GAAAGCCC CUGAUGAG GCCGUUAGGC CGAA ACGAACCA | 7584 |
| 714 | GUAGGGCU U UCCCCCAC | 185 | GUGGGGGA CUGAUGAG GCCGUUAGGC CGAA AGCCCUAC | 7585 |
| 715 | UAGGGCUU U CCCCCACU | 186 | AGUGGGGG CUGAUGAG GCCGUUAGGC CGAA AAGCCCUA | 7586 |
| 716 | AGGGCUUU C CCCCACUG | 187 | CAGUGGGG CUGAUGAG GCCGUUAGGC CGAA AAAGCCCU | 7587 |
| 726 | CCCACUGU C UGGCUUUC | 188 | GAAAGCCA CUGAUGAG GCCGUUAGGC CGAA ACAGUGGG | 7588 |
| 732 | GUCUGGCU U UCAGUUAU | 189 | AUAACUGA CUGAUGAG GCCGUUAGGC CGAA AGCCAGAC | 7589 |
| 733 | UCUGGCUU U CAGUUAUA | 190 | UAUAACUG CUGAUGAG GCCGUUAGGC CGAA AAGCCAGA | 7590 |
| 734 | CUGGCUUU C AGUUAUAU | 191 | AUAUAACU CUGAUGAG GCCGUUAGGC CGAA AAAGCCAG | 7591 |
| 738 | CUUUCAGU U AUAUGGAU | 192 | AUCCAUAU CUGAUGAG GCCGUUAGGC CGAA ACUGAAAG | 7592 |
| 739 | UUUCAGUU A UAUGGAUG | 193 | CAUCCAUA CUGAUGAG GCCGUUAGGC CGAA AACUGAAA | 7593 |
| 741 | UCAGUUAU A UGGAUGAU | 194 | AUCAUCCA CUGAUGAG GCCGUUAGGC CGAA AUAACUGA | 7594 |
| 755 | GAUGUGGU U UUGGGGGC | 195 | GCCCCCAA CUGAUGAG GCCGUUAGGC CGAA ACCACAUC | 7595 |
| 756 | AUGUGGUU U UGGGGGCC | 196 | GGCCCCCA CUGAUGAG GCCGUUAGGC CGAA AACCACAU | 7596 |
| 757 | UGUGGUUU U GGGGGCCA | 197 | UGGCCCCC CUGAUGAG GCCGUUAGGC CGAA AAACCACA | 7597 |
| 769 | GGCCAAGU C UGUACAAC | 198 | GUUGUACA CUGAUGAG GCCGUUAGGC CGAA ACUUGGCC | 7598 |
| 773 | AAGUCUGU A CAACAUCU | 199 | AGAUGUUG CUGAUGAG GCCGUUAGGC CGAA ACAGACUU | 7599 |
| 780 | UACAACAU C UUGAGUCC | 200 | GGACUCAA CUGAUGAG GCCGUUAGGC CGAA AUGUUGUA | 7600 |
| 782 | CAACAUCU U GAGUCCCU | 201 | AGGGACUC CUGAUGAG GCCGUUAGGC CGAA AGAUGUUG | 7601 |
| 787 | UCUUGAGU C CCUUUAUG | 202 | CAUAAAGG CUGAUGAG GCCGUUAGGC CGAA ACUCAAGA | 7602 |
| 791 | GAGUCCCU U UAUGCCGC | 203 | GCGGCAUA CUGAUGAG GCCGUUAGGC CGAA AGGGACUC | 7603 |
| 792 | AGUCCCUU U AUGCCGCU | 204 | AGCGGCAU CUGAUGAG GCCGUUAGGC CGAA AAGGGACU | 7604 |
| 793 | GUCCCUUU A UGCCGCUG | 205 | CAGCGGCA CUGAUGAG GCCGUUAGGC CGAA AAAGGGAC | 7605 |
| 803 | GCCGCUGU U ACCAAUUU | 206 | AAAUUGGU CUGAUGAG GCCGUUAGGC CGAA ACAGCGGC | 7606 |
| 804 | CCGCUGUU A CCAAUUUU | 207 | AAAAUUGG CUGAUGAG GCCGUUAGGC CGAA AACAGCGG | 7607 |
| 810 | UUACCAAU U UUCUUUUG | 208 | CAAAAGAA CUGAUGAG GCCGUUAGGC CGAA AUUGGUAA | 7608 |
| 811 | UACCAAUU U UCUUUUGU | 209 | ACAAAAGA CUGAUGAG GCCGUUAGGC CGAA AAUUGGUA | 7609 |
| 812 | ACCAAUUU U CUUUUGUC | 210 | GACAAAAG CUGAUGAG GCCGUUAGGC CGAA AAAUUGGU | 7610 |
| 813 | CCAAUUUU C UUUUGUCU | 211 | AGACAAAA CUGAUGAG GCCGUUAGGC CGAA AAAAUUGG | 7611 |
| 815 | AAUUUUCU U UUGUCUUU | 212 | AAAGACAA CUGAUGAG GCCGUUAGGC CGAA AGAAAAUU | 7612 |
| 816 | AUUUUCUU U UGUCUUUG | 213 | CAAAGACA CUGAUGAG GCCGUUAGGC CGAA AAGAAAAU | 7613 |
| 817 | UUUUCUUU U GUCUUUGG | 214 | CCAAAGAC CUGAUGAG GCCGUUAGGC CGAA AAAGAAAA | 7614 |
| 820 | UCUUUUGU C UUUGGGUA | 215 | UACCCAAA CUGAUGAG GCCGUUAGGC CGAA ACAAAAGA | 7615 |
| 822 | UUUUGUCU U UGGGUAUA | 216 | UAUACCCA CUGAUGAG GCCGUUAGGC CGAA AGACAAAA | 7616 |
| 823 | UUUGUCUU U GGGUAUAC | 217 | GUAUACCC CUGAUGAG GCCGUUAGGC CGAA AAGACAAA | 7617 |
| 828 | CUUUGGGU A UACAUUUA | 218 | UAAAUGUA CUGAUGAG GCCGUUAGGC CGAA ACCCAAAG | 7618 |
| 830 | UUGGGUAU A CAUUUAAA | 219 | UUUAAAUG CUGAUGAG GCCGUUAGGC CGAA AUACCCAA | 7619 |
| 834 | GUAUACAU U UAAACCCU | 220 | AGGGUUUA CUGAUGAG GCCGUUAGGC CGAA AUGUAUAC | 7620 |
| 835 | UAUACAUU U AAACCCUC | 221 | GAGGGUUU CUGAUGAG GCCGUUAGGC CGAA AAUGUAUA | 7621 |
| 836 | AUACAUUU A AACCCUCA | 222 | UGAGGGUU CUGAUGAG GCCGUUAGGC CGAA AAAUGUAU | 7622 |
| 843 | UAAACCCU C ACAAAACA | 223 | UGUUUUGU CUGAUGAG GCCGUUAGGC CGAA AGGGUUUA | 7623 |
| 865 | AUGGGGAU A UUCCCUUA | 224 | UAAGGGAA CUGAUGAG GCCGUUAGGC CGAA AUCCCCAU | 7624 |
| 867 | GGGGAUAU U CCCUUAAC | 225 | GUUAAGGG CUGAUGAG GCCGUUAGGC CGAA AUAUCCCC | 7625 |
| 868 | GGGAUAUU C CCUUAACU | 226 | AGUUAAGG CUGAUGAG GCCGUUAGGC CGAA AAUAUCCC | 7626 |
| 872 | UAUUCCCU U AACUUCAU | 227 | AUGAAGUU CUGAUGAG GCCGUUAGGC CGAA AGGGAAUA | 7627 |
| 873 | AUUCCCUU A ACUUCAUG | 228 | CAUGAAGU CUGAUGAG GCCGUUAGGC CGAA AAGGGAAU | 7628 |
| 877 | CCUUAACU U CAUGGGAU | 229 | AUCCCAUG CUGAUGAG GCCGUUAGGC CGAA AGUUAAGG | 7629 |
| 878 | CUUAACUU C AUGGGAUA | 230 | UAUCCCAU CUGAUGAG GCCGUUAGGC CGAA AAGUUAAG | 7630 |
| 886 | CAUGGGAU A UGUAAUUG | 231 | CAAUUACA CUGAUGAG GCCGUUAGGC CGAA AUCCCAUG | 7631 |
| 890 | GGAUAUGU A AUUGGGAG | 232 | CUCCCAAU CUGAUGAG GCCGUUAGGC CGAA ACAUAUCC | 7632 |
| 893 | UAUGUAAU U GGGAGUUG | 233 | CAACUCCC CUGAUGAG GCCGUUAGGC CGAA AUUACAUA | 7633 |
| 900 | UUGGGAGU U GGGGCACA | 234 | UGUGCCCC CUGAUGAG GCCGUUAGGC CGAA ACUCCCAA | 7634 |
| 910 | GGGCACAU U GCCACAGG | 235 | CCUGUGGC CUGAUGAG GCCGUUAGGC CGAA AUGUGCCC | 7635 |
| 924 | AGGAACAU A UUGUACAA | 236 | UUGUACAA CUGAUGAG GCCGUUAGGC CGAA AUGUUCCU | 7636 |
| 926 | GAACAUAU U GUACAAAA | 237 | UUUUGUAC CUGAUGAG GCCGUUAGGC CGAA AUAUGUUC | 7637 |
| 929 | CAUAUUGU A CAAAAAAU | 238 | AUUUUUUG CUGAUGAG GCCGUUAGGC CGAA ACAAUAUG | 7638 |
| 938 | CAAAAAAU C AAAAUGUG | 239 | CACAUUUU CUGAUGAG GCCGUUAGGC CGAA AUUUUUUG | 7639 |
| 948 | AAAUGUGU U UUAGGAAA | 240 | UUUCCUAA CUGAUGAG GCCGUUAGGC CGAA ACACAUUU | 7640 |
| 949 | AAUGUGUU U UAGGAAAC | 241 | GUUUCCUA CUGAUGAG GCCGUUAGGC CGAA AACACAUU | 7641 |
| 950 | AUGUGUUU U AGGAAACU | 242 | AGUUUCCU CUGAUGAG GCCGUUAGGC CGAA AAACACAU | 7642 |
| 951 | UGUGUUUU A GGAAACUU | 243 | AAGUUUCC CUGAUGAG GCCGUUAGGC CGAA AAAACACA | 7643 |
| 959 | AGGAAACU U CCUGUAAA | 244 | UUUACAGG CUGAUGAG GCCGUUAGGC CGAA AGUUUCCU | 7644 |
| 960 | GGAAACUU C CUGUAAAC | 245 | GUUUACAG CUGAUGAG GCCGUUAGGC CGAA AAGUUUCC | 7645 |
| 965 | CUUCCUGU A AACAGGCC | 246 | GGCCUGUU CUGAUGAG GCCGUUAGGC CGAA ACAGGAAG | 7646 |
| 975 | ACAGGCCU A UUGAUUGG | 247 | CCAAUCAA CUGAUGAG GCCGUUAGGC CGAA AGGCCUGU | 7647 |
| 977 | AGGCCUAU U GAUUGGAA | 248 | UUCCAAUC CUGAUGAG GCCGUUAGGC CGAA AUAGGCCU | 7648 |
| 981 | CUAUUGAU U GGAAAGUA | 249 | UACUUUCC CUGAUGAG GCCGUUAGGC CGAA AUCAAUAG | 7649 |
| 989 | UGGAAAGU A UGUCAACG | 250 | CGUUGACA CUGAUGAG GCCGUUAGGC CGAA ACUUUCCA | 7650 |
| 993 | AAGUAUGU C AACGAAUU | 251 | AAUUCGUU CUGAUGAG GCCGUUAGGC CGAA ACAUACUU | 7651 |
| 1001 | CAACGAAU U GUGGGUCU | 252 | AGACCCAC CUGAUGAG GCCGUUAGGC CGAA AUUCGUUG | 7652 |
| 1008 | UUGUGGGU C UUUUGGGG | 253 | CCCCAAAA CUGAUGAG GCCGUUAGGC CGAA ACCCACAA | 7653 |
| 1010 | GUGGGUCU U UUGGGGUU | 254 | AACCCCAA CUGAUGAG GCCGUUAGGC CGAA AGACCCAC | 7654 |
| 1011 | UGGGUCUU U UGGGGUUU | 255 | AAACCCCA CUGAUGAG GCCGUUAGGC CGAA AAGACCCA | 7655 |
| 1012 | GGGUCUUU U GGGGUUUG | 256 | CAAACCCC CUGAUGAG GCCGUUAGGC CGAA AAAGACCC | 7656 |
| 1018 | UUUGGGGU U UGCCGCCC | 257 | GGGCGGCA CUGAUGAG GCCGUUAGGC CGAA ACCCCAAA | 7657 |
| 1019 | UUGGGGUU U GCCGCCCC | 258 | GGGGCGGC CUGAUGAG GCCGUUAGGC CGAA AACCCCAA | 7658 |
| 1029 | CCGCCCCU U UCACGCAA | 259 | UUGCGUGA CUGAUGAG GCCGUUAGGC CGAA AGGGGCGG | 7659 |
| 1030 | CGCCCCUU U CACGCAAU | 260 | AUUGCGUG CUGAUGAG GCCGUUAGGC CGAA AAGGGGCG | 7660 |
| 1031 | GCCCCUUU C ACGCAAUG | 261 | CAUUGCGU CUGAUGAG GCCGUUAGGC CGAA AAAGGGGC | 7661 |
| 1045 | AUGUGGAU A UUCUGCUU | 262 | AAGCAGAA CUGAUGAG GCCGUUAGGC CGAA AUCCACAU | 7662 |
| 1047 | GUGGAUAU U CUGCUUUA | 263 | UAAAGCAG CUGAUGAG GCCGUUAGGC CGAA AUAUCCAC | 7663 |
| 1048 | UGGAUAUU C UGCUUUAA | 264 | UUAAAGCA CUGAUGAG GCCGUUAGGC CGAA AAUAUCCA | 7664 |
| 1053 | AUUCUGCU U UAAUGCCU | 265 | AGGCAUUA CUGAUGAG GCCGUUAGGC CGAA AGCAGAAU | 7665 |
| 1054 | UUCUGCUU U AAUGCCUU | 266 | AAGGCAUU CUGAUGAG GCCGUUAGGC CGAA AAGCAGAA | 7666 |
| 1055 | UCUGCUUU A AUGCCUUU | 267 | AAAGGCAU CUGAUGAG GCCGUUAGGC CGAA AAAGCAGA | 7667 |
| 1062 | UAAUGCCU U UAUAUGCA | 268 | UGCAUAUA CUGAUGAG GCCGUUAGGC CGAA AGGCAUUA | 7668 |
| 1063 | AAUGCCUU U AUAUGCAU | 269 | AUGCAUAU CUGAUGAG GCCGUUAGGC CGAA AAGGCAUU | 7669 |
| 1064 | AUGCCUUU A UAUGCAUG | 270 | CAUGCAUA CUGAUGAG GCCGUUAGGC CGAA AAAGGCAU | 7670 |
| 1066 | GCCUUUAU A UGCAUGCA | 271 | UGCAUGCA CUGAUGAG GCCGUUAGGC CGAA AUAAAGGC | 7671 |
| 1076 | GCAUGCAU A CAAGCAAA | 272 | UUUGCUUG CUGAUGAG GCCGUUAGGC CGAA AUGCAUGC | 7672 |
| 1092 | AACAGGCU U UUACUUUC | 273 | GAAAGUAA CUGAUGAG GCCGUUAGGC CGAA AGCCUGUU | 7673 |
| 1093 | ACAGGCUU U UACUUUCU | 274 | AGAAAGUA CUGAUGAG GCCGUUAGGC CGAA AAGCCUGU | 7674 |
| 1094 | CAGGCUUU U ACUUUCUC | 275 | GAGAAAGU CUGAUGAG GCCGUUAGGC CGAA AAAGCCUG | 7675 |
| 1095 | AGGCUUUU A CUUUCUCG | 276 | CGAGAAAG CUGAUGAG GCCGUUAGGC CGAA AAAAGCCU | 7676 |
| 1098 | CUUUUACU U UCUCGCCA | 277 | UGGCGAGA CUGAUGAG GCCGUUAGGC CGAA AGUAAAAG | 7677 |
| 1099 | UUUUACUU U CUCGCCAA | 278 | UUGGCGAG CUGAUGAG GCCGUUAGGC CGAA AAGUAAAA | 7678 |
| 1100 | UUUACUUU C UCGCCAAC | 279 | GUUGGCGA CUGAUGAG GCCGUUAGGC CGAA AAAGUAAA | 7679 |
| 1102 | UACUUUCU C GCCAACUU | 280 | AAGUUGGC CUGAUGAG GCCGUUAGGC CGAA AGAAAGUA | 7680 |
| 1110 | CGCCAACU U ACAAGGCC | 281 | GGCCUUGU CUGAUGAG GCCGUUAGGC CGAA AGUUGGCG | 7681 |
| 1111 | GCCAACUU A CAAGGCCU | 282 | AGGCCUUG CUGAUGAG GCCGUUAGGC CGAA AAGUUGGC | 7682 |
| 1120 | CAAGGCCU U UCUAAGUA | 283 | UACUUAGA CUGAUGAG GCCGUUAGGC CGAA AGGCCUUG | 7683 |
| 1121 | AAGGCCUU U CUAAGUAA | 284 | UUACUUAG CUGAUGAG GCCGUUAGGC CGAA AAGGCCUU | 7684 |
| 1122 | AGGCCUUU C UAAGUAAA | 285 | UUUACUUA CUGAUGAG GCCGUUAGGC CGAA AAAGGCCU | 7685 |
| 1124 | GCCUUUCU A AGUAAACA | 286 | UGUUUACU CUGAUGAG GCCGUUAGGC CGAA AGAAAGGC | 7686 |
| 1128 | UUCUAAGU A AACAGUAU | 287 | AUACUGUU CUGAUGAG GCCGUUAGGC CGAA ACUUAGAA | 7687 |
| 1135 | UAAACAGU A UGUGAACC | 288 | GGUUCACA CUGAUGAG GCCGUUAGGC CGAA ACUGUUUA | 7688 |
| 1145 | GUGAACCU U UACCCCGU | 289 | ACGGGGUA CUGAUGAG GCCGUUAGGC CGAA AGGUUCAC | 7689 |
| 1146 | UGAACCUU U ACCCCGUU | 290 | AACGGGGU CUGAUGAG GCCGUUAGGC CGAA AAGGUUCA | 7690 |
| 1147 | GAACCUUU A CCCCGUUG | 291 | CAACGGGG CUGAUGAG GCCGUUAGGC CGAA AAAGGUUC | 7691 |
| 1154 | UACCCCGU U GCUCGGCA | 292 | UGCCGAGC CUGAUGAG GCCGUUAGGC CGAA ACGGGGUA | 7692 |
| 1158 | CCGUUGCU C GGCAACGG | 293 | CCGUUGCC CUGAUGAG GCCGUUAGGC CGAA AGCAACGG | 7693 |
| 1173 | GGCCUGGU C UAUGCCAA | 294 | UUGGCAUA CUGAUGAG GCCGUUAGGC CGAA ACCAGGCC | 7694 |
| 1175 | CCUGGUCU A UGCCAAGU | 295 | ACUUGGCA CUGAUGAG GCCGUUAGGC CGAA AGACCAGG | 7695 |
| 1186 | CCAAGUGU U UGCUGACG | 296 | CGUCAGCA CUGAUGAG GCCGUUAGGC CGAA ACACUUGG | 7696 |
| 1187 | CAAGUGUU U GCUGACGC | 297 | GCGUCAGC CUGAUGAG GCCGUUAGGC CGAA AACACUUG | 7697 |
| 1209 | CCACUGGU U GGGGCUUG | 298 | CAAGCCCC CUGAUGAG GCCGUUAGGC CGAA ACCAGUGG | 7698 |
| 1216 | UUGGGGCU U GGCCAUAG | 299 | CUAUGGCC CUGAUGAG GCCGUUAGGC CGAA AGCCCCAA | 7699 |
| 1223 | UUGGCCAU A GGCCAUCA | 300 | UGAUGGCC CUGAUGAG GCCGUUAGGC CGAA AUGGCCAA | 7700 |
| 1230 | UAGGCCAU C AGCGCAUG | 301 | CAUGCGCU CUGAUGAG GCCGUUAGGC CGAA AUGGCCUA | 7701 |
| 1249 | UGGAACCU U UGUGUCUC | 302 | GAGACACA CUGAUGAG GCCGUUAGGC CGAA AGGUUCCA | 7702 |
| 1250 | GGAACCUU U GUGUCUCC | 303 | GGAGACAC CUGAUGAG GCCGUUAGGC CGAA AAGGUUCC | 7703 |
| 1255 | CUUUGUGU C UCCUCUGC | 304 | GCAGAGGA CUGAUGAG GCCGUUAGGC CGAA ACACAAAG | 7704 |
| 1257 | UUGUGUCU C CUCUGCCG | 305 | CGGCAGAG CUGAUGAG GCCGUUAGGC CGAA AGACACAA | 7705 |
| 1260 | UGUCUCCU C UGCCGAUC | 306 | GAUCGGCA CUGAUGAG GCCGUUAGGC CGAA AGGAGACA | 7706 |
| 1268 | CUGCCGAU C CAUACCGC | 307 | GCGGUAUG CUGAUGAG GCCGUUAGGC CGAA AUCGGCAG | 7707 |
| 1272 | CGAUCCAU A CCGCGGAA | 308 | UUCCGCGG CUGAUGAG GCCGUUAGGC CGAA AUGGAUCG | 7708 |
| 1283 | GCGGAACU C CUAGCCGC | 309 | GCGGCUAG CUGAUGAG GCCGUUAGGC CGAA AGUUCCGC | 7709 |
| 1286 | GAACUCCU A GCCGCUUG | 310 | CAAGCGGC CUGAUGAG GCCGUUAGGC CGAA AGGAGUUC | 7710 |
| 1293 | UAGCCGCU U GUUUUGCU | 311 | AGCAAAAC CUGAUGAG GCCGUUAGGC CGAA AGCGGCUA | 7711 |
| 1296 | CCGCUUGU U UUGCUCGC | 312 | GCGAGCAA CUGAUGAG GCCGUUAGGC CGAA ACAAGCGG | 7712 |
| 1297 | CGCUUGUU U UGCUCGCA | 313 | UGCGAGCA CUGAUGAG GCCGUUAGGC CGAA AACAAGCG | 7713 |
| 1298 | GCUUGUUU U GCUCGCAG | 314 | CUGCGAGC CUGAUGAG GCCGUUAGGC CGAA AAACAAGC | 7714 |
| 1302 | GUUUUGCU C GCAGCAGG | 315 | CCUGCUGC CUGAUGAG GCCGUUAGGC CGAA AGCAAAAC | 7715 |
| 1312 | CAGCAGGU C UGGGGCAA | 316 | UUGCCCCA CUGAUGAG GCCGUUAGGC CGAA ACCUGCUG | 7716 |
| 1325 | GCAAAACU C AUCGGGAC | 317 | GUCCCGAU CUGAUGAG GCCGUUAGGC CGAA AGUUUUGC | 7717 |
| 1328 | AAACUCAU C GGGACUGA | 318 | UCAGUCCC CUGAUGAG GCCGUUAGGC CGAA AUGAGUUU | 7718 |
| 1341 | CUGACAAU U CUGUCGUG | 319 | CACGACAG CUGAUGAG GCCGUUAGGC CGAA AUUGUCAG | 7719 |
| 1342 | UGACAAUU C UGUCGUGC | 320 | GCACGACA CUGAUGAG GCCGUUAGGC CGAA AAUUGUCA | 7720 |
| 1346 | AAUUCUGU C GUGCUCUC | 321 | GAGAGCAC CUGAUGAG GCCGUUAGGC CGAA ACAGAAUU | 7721 |
| 1352 | GUCGUGCU C UCCCGCAA | 322 | UUGCGGGA CUGAUGAG GCCGUUAGGC CGAA AGCACGAC | 7722 |
| 1354 | CGUGCUCU C CCGCAAAU | 323 | AUUUGCGG CUGAUGAG GCCGUUAGGC CGAA AGAGCACG | 7723 |
| 1363 | CCGCAAAU A UACAUCAU | 324 | AUGAUGUA CUGAUGAG GCCGUUAGGC CGAA AUUUGCGG | 7724 |
| 1365 | GCAAAUAU A CAUCAUUU | 325 | AAAUGAUG CUGAUGAG GCCGUUAGGC CGAA AUAUUUGC | 7725 |
| 1369 | AUAUACAU C AUUUCCAU | 326 | AUGGAAAU CUGAUGAG GCCGUUAGGC CGAA AUGUAUAU | 7726 |
| 1372 | UACAUCAU U UCCAUGGC | 327 | GCCAUGGA CUGAUGAG GCCGUUAGGC CGAA AUGAUGUA | 7727 |
| 1373 | ACAUCAUU U CCAUGGCU | 328 | AGCCAUGG CUGAUGAG GCCGUUAGGC CGAA AAUGAUGU | 7728 |
| 1374 | CAUCAUUU C CAUGGCUG | 329 | CAGCCAUG CUGAUGAG GCCGUUAGGC CGAA AAAUGAUG | 7729 |
| 1385 | UGGCUGCU A GGCUGUGC | 330 | GCACAGCC CUGAUGAG GCCGUUAGGC CGAA AGCAGCCA | 7730 |
| 1406 | AACUGGAU C CUACGCGG | 331 | CCGCGUAG CUGAUGAG GCCGUUAGGC CGAA AUCCAGUU | 7731 |
| 1409 | UGGAUCCU A CGCGGGAC | 332 | GUCCCGCG CUGAUGAG GCCGUUAGGC CGAA AGGAUCCA | 7732 |
| 1420 | CGGGACGU C CUUUGUUU | 333 | AAACAAAG CUGAUGAG GCCGUUAGGC CGAA ACGUCCCG | 7733 |
| 1423 | GACGUCCU U UGUUUACG | 334 | CGUAAACA CUGAUGAG GCCGUUAGGC CGAA AGGACGUC | 7734 |
| 1424 | ACGUCCUU U GUUUACGU | 335 | ACGUAAAC CUCAUGAG GCCGUUAGGC CGAA AAGGACGU | 7735 |
| 1427 | UCCUUUGU U UACGUCCC | 336 | GGGACGUA CUGAUGAG GCCGUUAGGC CGAA ACAAAGGA | 7736 |
| 1428 | CCUUUGUU U ACGUCCCG | 337 | CGGGACGU CUGAUGAG GCCGUUAGGC CGAA AACAAAGG | 7737 |
| 1429 | CUUUGUUU A CGUCCCGU | 338 | ACGGGACG CUGAUGAG GCCGUUAGGC CGAA AAACAAAG | 7738 |
| 1433 | GUUUACGU C CCGUCGGC | 339 | GCCGACGG CUGAUGAG GCCGUUAGGC CGAA ACGUAAAC | 7739 |
| 1438 | CGUCCCGU C GGCGCUGA | 340 | UCAGCGCC CUGAUGAG GCCGUUAGGC CGAA ACGGGACG | 7740 |
| 1449 | CGCUGAAU C CCGCGGAC | 341 | GUCCGCGG CUGAUGAG GCCGUUAGGC CGAA AUUCAGCG | 7741 |
| 1465 | CGACCCCU C CCGGGGCC | 342 | GGCCCCGG CUGAUGAG GCCGUUAGGC CGAA AGGGGUCG | 7742 |
| 1477 | GGGCCGCU U GGGGCUCU | 343 | AGAGCCCC CUGAUGAG GCCGUUAGGC CGAA AGCGGCCC | 7743 |
| 1484 | UUGGGGCU C UACCGCCC | 344 | GGGCGGUA CUGAUGAG GCCGUUAGGC CGAA AGCCCCAA | 7744 |
| 1486 | GGGGCUCU A CCGCCCGC | 345 | GCGGGCGG CUGAUGAG GCCGUUAGGC CGAA AGAGCCCC | 7745 |
| 1496 | CGCCCGCU U CUCCGCCU | 346 | AGGCGGAG CUGAUGAG GCCGUUAGGC CGAA AGCGGGCG | 7746 |
| 1497 | GCCCGCUU C UCCGCCUA | 347 | UAGGCGGA CUGAUGAG GCCGUUAGGC CGAA AAGCGGGC | 7747 |
| 1499 | CCGCUUCU C CGCCUAUU | 348 | AAUAGGCG CUGAUGAG GCCGUUAGGC CGAA AGAAGCGG | 7748 |
| 1505 | CUCCGCCU A UUGUACCG | 349 | CGGUACAA CUGAUGAG GCCGUUAGGC CGAA AGGCGGAG | 7749 |
| 1507 | CCGCCUAU U GUACCGAC | 350 | GUCGGUAC CUGAUGAG GCCGUUAGGC CGAA AUAGGCGG | 7750 |
| 1510 | CCUAUUGU A CCGACCGU | 351 | ACGGUCGG CUGAUGAG GCCGUUAGGC CGAA ACAAUAGG | 7751 |
| 1519 | CCGACCGU C CACGGGGC | 352 | GCCCCGUG CUGAUGAG GCCGUUAGGC CGAA ACGGUCGG | 7752 |
| 1534 | GCGCACCU C UCUUUACG | 353 | CGUAAAGA CUGAUGAG GCCGUUAGGC CGAA AGGUGCGC | 7753 |
| 1536 | GCACCUCU C UUUACGCG | 354 | CGCGUAAA CUGAUGAG GCCGUUAGGC CGAA AGAGGUGC | 7754 |
| 1538 | ACCUCUCU U UACGCGGA | 355 | UCCGCGUA CUGAUGAG GCCGUUAGGC CGAA AGAGAGGU | 7755 |
| 1539 | CCUCUCUU U ACGCGGAC | 356 | GUCCGCGU CUGAUGAG GCCGUUAGGC CGAA AAGAGAGG | 7756 |
| 1540 | CUCUCUUU A CGCGGACU | 357 | AGUCCGCG CUGAUGAG GCCGUUAGGC CGAA AAAGAGAG | 7757 |
| 1549 | CGCGGACU C CCCGUCUG | 358 | CAGACGGG CUGAUGAG GCCGUUAGGC CGAA AGUCCGCG | 7758 |
| 1555 | CUCCCCGU C UGUGCCUU | 359 | AAGGCACA CUGAUGAG GCCGUUAGGC CGAA ACGGGGAG | 7759 |
| 1563 | CUGUGCCU U CUCAUCUG | 360 | CAGAUGAG CUGAUGAG GCCGUUAGGC CGAA AGGCACAG | 7760 |
| 1564 | UGUGCCUU C UCAUCUGC | 361 | GCAGAUGA CUGAUGAG GCCGUUAGGC CGAA AAGGCACA | 7761 |
| 1566 | UGCCUUCU C AUCUGCCG | 362 | CGGCAGAU CUGAUGAG GCCGUUAGGC CGAA AGAAGGCA | 7762 |
| 1569 | CUUCUCAU C UGCCGGAC | 363 | GUCCGGCA CUGAUGAG GCCGUUAGGC CGAA AUGAGAAG | 7763 |
| 1588 | UGUGCACU U CGCUUCAC | 364 | GUGAAGCG CUGAUGAG GCCGUUAGGC CGAA AGUGCACA | 7764 |
| 1589 | GUGCACUU C GCUUCACC | 365 | GGUGAAGC CUGAUGAG GCCGUUAGGC CGAA AAGUGCAC | 7765 |
| 1593 | ACUUCGCU U CACCUCUG | 366 | CAGAGGUG CUGAUGAG GCCGUUAGGC CGAA AGCGAAGU | 7766 |
| 1594 | CUUCGCUU C ACCUCUGC | 367 | GCAGAGGU CUGAUGAG GCCGUUAGGC CGAA AAGCGAAG | 7767 |
| 1599 | CUUCACCU C UGCACGUC | 368 | GACGUGCA CUGAUGAG GCCGUUAGGC CGAA AGGUGAAG | 7768 |
| 1607 | CUGCACGU C GCAUGGAG | 369 | CUCCAUGC CUGAUGAG GCCGUUAGGC CGAA ACGUGCAG | 7769 |
| 1651 | CCCAAGGU C UUGCAUAA | 370 | UUAUGCAA CUGAUGAG GCCGUUAGGC CGAA ACCUUGGG | 7770 |
| 1653 | CAAGGUCU U GCAUAAGA | 371 | UCUUAUGC CUGAUGAG GCCGUUAGGC CGAA AGACCUUG | 7771 |
| 1658 | UCUUGCAU A AGAGGACU | 372 | AGUCCUCU CUGAUGAG GCCGUUAGGC CGAA AUGCAAGA | 7772 |
| 1667 | AGAGGACU C UUGGACUU | 373 | AAGUCCAA CUGAUGAG GCCGUUAGGC CGAA AGUCCUCU | 7773 |
| 1669 | AGGACUCU U GGACUUUC | 374 | GAAAGUCC CUGAUGAG GCCGUUAGGC CGAA AGAGUCCU | 7774 |
| 1675 | CUUGGACU U UCAGCAAU | 375 | AUUGCUGA CUGAUGAG GCCGUUAGGC CGAA AGUCCAAG | 7775 |
| 1676 | UUGGACUU U CAGCAAUG | 376 | CAUUGCUG CUGAUGAG GCCGUUAGGC CGAA AAGUCCAA | 7776 |
| 1677 | UGGACUUU C AGCAAUGU | 377 | ACAUUGCU CUGAUGAG GCCGUUAGGC CGAA AAAGUCCA | 7777 |
| 1686 | AGCAAUGU C AACGACCG | 378 | CGGUCGUU CUGAUGAG GCCGUUAGGC CGAA ACAUUGCU | 7778 |
| 1699 | ACCGACCU U GAGGCAUA | 379 | UAUGCCUC CUGAUGAG GCCGUUAGGC CGAA AGGUCGGU | 7779 |
| 1707 | UGAGGCAU A CUUCAAAG | 380 | CUUUGAAG CUGAUGAG GCCGUUAGGC CGAA AUGCCUCA | 7780 |
| 1710 | GGCAUACU U CAAAGACU | 381 | AGUCUUUG CUGAUGAG GCCGUUAGGC CGAA AGUAUGCC | 7781 |
| 1711 | GCAUACUU C AAAGACUG | 382 | CAGUCUUU CUGAUGAG GCCGUUAGGC CGAA AAGUAUGC | 7782 |
| 1725 | CUGUGUGU U UAAUGAGU | 383 | ACUCAUUA CUGAUGAG GCCGUUAGGC CGAA ACACACAG | 7783 |
| 1726 | UGUGUGUU U AAUGAGUG | 384 | CACUCAUU CUGAUGAG GCCGUUAGGC CGAA AACACACA | 7784 |
| 1727 | GUGUGUUU A AUGAGUGG | 385 | CCACUCAU CUGAUGAG GCCGUUAGGC CGAA AAACACAC | 7785 |
| 1743 | GGAGGAGU U GGGGGAGG | 386 | CCUCCCCC CUGAUGAG GCCGUUAGGC CGAA ACUCCUCC | 7786 |
| 1756 | GAGGAGGU U AGGUUAAA | 387 | UUUAACCU CUGAUGAG GCCGUUAGGC CGAA ACCUCCUC | 7787 |
| 1757 | AGGAGGUU A GGUUAAAG | 388 | CUUUAACC CUGAUGAG GCCGUUAGGC CGAA AACCUCCU | 7788 |
| 1761 | GGUUAGGU U AAAGGUCU | 389 | AGACCUUU CUGAUGAG GCCGUUAGGC CGAA ACCUAACC | 7789 |
| 1762 | GUUAGGUU A AAGGUCUU | 390 | AAGACCUU CUGAUGAG GCCGUUAGGC CGAA AACCUAAC | 7790 |
| 1768 | UUAAAGGU C UUUGUACU | 391 | AGUACAAA CUGAUGAG GCCGUUAGGC CGAA ACCUUUAA | 7791 |
| 1770 | AAAGGUCU U UGUACUAG | 392 | CUAGUACA CUGAUGAG GCCGUUAGGC CGAA AGACCUUU | 7792 |
| 1771 | AAGGUCUU U GUACUAGG | 393 | CCUAGUAC CUGAUGAG GCCGUUAGGC CGAA AAGACCUU | 7793 |
| 1774 | GUCUUUGU A CUAGGAGG | 394 | CCUCCUAG CUGAUGAG GCCGUUAGGC CGAA ACAAAGAC | 7794 |
| 1777 | UUUGUACU A GGAGGCUG | 395 | CAGCCUCC CUGAUGAG GCCGUUAGGC CGAA AGUACAAA | 7795 |
| 1787 | GAGGCUGU A GGCAUAAA | 396 | UUUAUGCC CUGAUGAG GCCGUUAGGC CGAA ACAGCCUC | 7796 |
| 1793 | GUAGGCAU A AAUUGGUG | 397 | CACCAAUU CUGAUGAG GCCGUUAGGC CGAA AUGCCUAC | 7797 |
| 1797 | GCAUAAAU U GGUGUGUU | 398 | AACACACC CUGAUGAG GCCGUUAGGC CGAA AUUUAUGC | 7798 |
| 1805 | UGGUGUGU U CACCAGCA | 399 | UGCUGGUG CUGAUGAG GCCGUUAGGC CGAA ACACACCA | 7799 |
| 1806 | GGUGUGUU C ACCAGCAC | 400 | GUGCUGGU CUGAUGAG GCCGUUAGGC CGAA AACACACC | 7800 |
| 1824 | AUGCAACU U UUUCACCU | 401 | AGGUGAAA CUGAUGAG GCCGUUAGGC CGAA AGUUGCAU | 7801 |
| 1825 | UGCAACUU U UUCACCUC | 402 | GAGGUGAA CUGAUGAG GCCGUUAGGC CGAA AAGUUGCA | 7802 |
| 1826 | GCAACUUU U UCACCUCU | 403 | AGAGGUGA CUGAUGAG GCCGUuAGGC CGAA AAAGUUGC | 7803 |
| 1827 | CAACUUUU U CACCUCUG | 404 | CAGAGGUG CUGAUGAG GCCGUUAGGC CGAA AAAAGUUG | 7804 |
| 1828 | AACUUUUU C ACCUCUGC | 405 | GCAGAGGU CUGAUGAG GCCGUUAGGC CGAA AAAAAGUU | 7805 |
| 1833 | UUUCACCU C UGCCUAAU | 406 | AUUAGGCA CUGAUGAG GCCGUUAGGC CGAA AGGUGAAA | 7806 |
| 1839 | CUCUGCCU A AUCAUCUC | 407 | GAGAUGAU CUGAUGAG GCCGUUAGGC CGAA AGGCAGAG | 7807 |
| 1842 | UGCCUAAU C AUCUCAUG | 408 | CAUGAGAU CUGAUGAG GCCGUUAGGC CGAA AUUAGGCA | 7808 |
| 1845 | CUAAUCAU C UCAUGUUC | 409 | GAACAUGA CUGAUGAG GCCGUUAGGC CGAA AUGAUUAG | 7809 |
| 1847 | AAUCAUCU C AUGUUCAU | 410 | AUGAACAU CUGAUGAG GCCGUUAGGC CGAA AGAUGAUU | 7810 |
| 1852 | UCUCAUGU U CAUGUCCU | 411 | AGGACAUG CUGAUGAG GCCGUUAGGC CGAA ACAUGAGA | 7811 |
| 1853 | CUCAUGUU C AUGUCCUA | 412 | UAGGACAU CUGAUGAG GCCGUUAGGC CGAA AACAUGAG | 7812 |
| 1858 | GUUCAUGU C CUACUGUU | 413 | AACAGUAG CUGAUGAG GCCGUUAGGC CGAA ACAUGAAC | 7813 |
| 1861 | CAUGUCCU A CUGUUCAA | 414 | UUGAACAG CUGAUGAG GCCGUUAGGC CGAA AGGACAUG | 7814 |
| 1866 | CCUACUGU U CAAGCCUC | 415 | GAGGCUUG CUGAUGAG GCCGUUAGGC CGAA ACAGUAGG | 7815 |
| 1867 | CUACUGUU C AAGCCUCC | 416 | GGAGGCUU CUGAUGAG GCCGUUAGGC CGAA AACAGUAG | 7816 |
| 1874 | UCAAGCCU C CAAGCUGU | 417 | ACAGCUUG CUGAUGAG GCCGUUAGGC CGAA AGGCUUGA | 7817 |
| 1887 | CUGUGCCU U GGGUGGCU | 418 | AGCCACCC CUGAUGAG GCCGUUAGGC CGAA AGGCACAG | 7818 |
| 1896 | GGGUGGCU U UGGGGCAU | 419 | AUGCCCCA CUGAUGAG GCCGUUAGGC CGAA AGCCACCC | 7819 |
| 1897 | GGUGGCUU U GGGGCAUG | 420 | CAUGCCCC CUGAUGAG GCCGUUAGGC CGAA AAGCCACC | 7820 |
| 1911 | AUGGACAU U GACCCGUA | 421 | UACGGGUC CUGAUGAG GCCGUUAGGC CGAA AUGUCCAU | 7821 |
| 1919 | UGACCCGU A UAAAGAAU | 422 | AUUCUUUA CUGAUGAG GCCGUUAGGC CGAA ACGGGUCA | 7822 |
| 1921 | ACCCGUAU A AAGAAUUU | 423 | AAAUUCUU CUGAUGAG GCCGUUAGGC CGAA AUACGGGU | 7823 |
| 1928 | UAAAGAAU U UGGAGCUU | 424 | AAGCUCCA CUGAUGAG GCCGUUAGGC CGAA AUUCUUUA | 7824 |
| 1929 | AAAGAAUU U GGAGCUUC | 425 | GAAGCUCC CUGAUGAG GCCGUUAGGC CGAA AAUUCUUU | 7825 |
| 1936 | UUGGAGCU U CUGUGGAG | 426 | CUCCACAG CUGAUGAG GCCGUUAGGC CGAA AGCUCCAA | 7826 |
| 1937 | UGGAGCUU C UGUGGAGU | 427 | ACUCCACA CUGAUGAG GCCGUUAGGC CGAA AAGCUCCA | 7827 |
| 1946 | UGUGGAGU U ACUCUCUU | 428 | AAGAGAGU CUGAUGAG GCCGUUAGGC CGAA ACUCCACA | 7828 |
| 1947 | GUGGAGUU A CUCUCUUU | 429 | AAAGAGAG CUGAUGAG GCCGUUAGGC CGAA AACUCCAC | 7829 |
| 1950 | GAGUUACU C UCUUUUUU | 430 | AAAAAAGA CUGAUGAG GCCGUUAGGC CGAA AGUAACUC | 7830 |
| 1952 | GUUACUCU C UUUUUUGC | 431 | GCAAAAAA CUGAUGAG GCCGUUAGGC CGAA AGAGUAAC | 7831 |
| 1954 | UACUCUCU U UUUUGCCU | 432 | AGGCAAUU CUGAUGAG GCCGUUAGGC CGAA AGAGAGUA | 7832 |
| 1955 | ACUCUCUU U UUUGCCUU | 433 | AAGGCAAA CUGAUGAG GCCGUUAGGC CGAA AAGAGAGU | 7833 |
| 1956 | CUCUCUUU U UUGCCUUC | 434 | GAAGGCAA CUGAUGAG GCCGUUAGGC CGAA AAAGAGAG | 7834 |
| 1957 | UCUCUUUU U UGCCUUCU | 435 | AGAAGGCA CUGAUGAG GCCGUUAGGC CGAA AAAAGAGA | 7835 |
| 1958 | CUCUUUUU U GCCUUCUG | 436 | CAGAAGGC CUGAUGAG GCCGUUAGGC CGAA AAAAAGAG | 7836 |
| 1963 | UUUUGCCU U CUGACUUC | 437 | GAACUCAG CUGAUGAG GCCGUUAGGC CGAA AGGCAAAA | 7837 |
| 1964 | UUUGCCUU C UGACUUCU | 438 | AGAAGUCA CUGAUGAG GCCGUUAGGC CGAA AAGGCAAA | 7838 |
| 1970 | UUCUGACU U CUUUCCUU | 439 | AAGGAAAG CUGAUGAG GCCGUUAGGC CGAA AGUCAGAA | 7839 |
| 1971 | UCUGACUU C UUUCCUUC | 440 | GAAGGAAA CUGAUGAG GCCGUUAGGC CGAA AAGUCAGA | 7840 |
| 1973 | UGACUUCU U UCCUUCUA | 441 | UAGAAGGA CUGAUGAG GCCGUUAGGC CGAA AGAAGUCA | 7841 |
| 1974 | GACUUCUU U CCUUCUAU | 442 | AUAGAAGG CUGAUGAG GCCGUUAGGC CGAA AAGAAGUC | 7842 |
| 1975 | ACUUCUUU C CUUCUAUU | 443 | AAUAGAAG CUGAUGAG GCCGUUAGGC CGAA AAAGAAGU | 7843 |
| 1978 | UCUUUCCU U CUAUUCGA | 444 | UCGAAUAG CUGAUGAG GCCGUUAGGC CGAA AGGAAAGA | 7844 |
| 1979 | CUUUCCUU C UAUUCGAG | 445 | CUCGAAUA CUGAUGAG GCCGUUAGGC CGAA AAGGAAAG | 7845 |
| 1981 | UUCCUUCU A UUCGAGAU | 446 | AUCUCGAA CUGAUGAG GCCGUUAGGC CGAA AGAAGGAA | 7846 |
| 1983 | CCUUCUAU U CGAGAUCU | 447 | AGAUCUCG CUGAUGAG GCCGUUAGGC CGAA AUAGAAGG | 7847 |
| 1984 | CUUCUAUU C GAGAUCUC | 448 | GAGAUCUC CUGAUGAG GCCGUUAGGC CGAA AAUAGAAG | 7848 |
| 1990 | UUCGAGAU C UCCUCGAC | 449 | GUCGAGGA CUGAUGAG GCCGUUAGGC CGAA AUCUCGAA | 7849 |
| 1992 | CGAGAUCU C CUCGACAC | 450 | GUGUCGAG CUGAUGAG GCCGUUAGGC CGAA AGAUCUCG | 7850 |
| 1995 | GAUCUCCU C GACACCGC | 451 | GCGGUGUC CUGAUGAG GCCGUUAGGC CGAA AGGAGAUC | 7851 |
| 2006 | CACCGCCU C UGCUCUGU | 452 | ACAGAGCA CUGAUGAG GCCGUUAGGC CGAA AGGCGGUG | 7852 |
| 2011 | CCUCUGCU C UGUAUCGG | 453 | CCGAUACA CUGAUGAG GCCGUUAGGC CGAA AGCAGAGG | 7853 |
| 2015 | UGCUCUGU A UCGGGGGG | 454 | CCCCCCGA CUGAUGAG GCCGUUAGGC CGAA ACAGAGCA | 7854 |
| 2017 | CUCUGUAU C GGGGGGCC | 455 | GGCCCCCC CUGAUGAG GCCGUUAGGC CGAA AUACAGAG | 7855 |
| 2027 | GGGGGCCU U AGAGUCUC | 456 | GAGACUCU CUGAUGAG GCCGUUAGGC CGAA AGGCCCCC | 7856 |
| 2028 | GGGGCCUU A GAGUCUCC | 457 | GGAGACUC CUGAUGAG GCCGUUAGGC CGAA AAGGCCCC | 7857 |
| 2033 | CUUAGAGU C UCCGGAAC | 458 | GUUCCGGA CUGAUGAG GCCGUUAGGC CGAA ACUCUAAG | 7858 |
| 2035 | UAGAGUCU C CGGAACAU | 459 | AUGUUCCG CUGAUGAG GCCGUUAGGC CGAA AGACUCUA | 7859 |
| 2044 | CGGAACAU U GUUCACCU | 460 | AGGUGAAC CUGAUGAG GCCGUUAGGC CGAA AUGUUCCG | 7860 |
| 2047 | AACAUUGU U CACCUCAC | 461 | GUGAGGUG CUGAUGAG GCCGUUAGGC CGAA ACAAUGUU | 7861 |
| 2048 | ACAUUGUU C ACCUCACC | 462 | GGUGAGGU CUGAUGAG GCCGUUAGGC CGAA AACAAUGU | 7862 |
| 2053 | GUUCACCU C ACCAUACG | 463 | CGUAUGGU CUGAUGAG GCCGUUAGGC CGAA AGGUGAAC | 7863 |
| 2059 | CUCACCAU A CGGCACUC | 464 | GAGUGCCG CUGAUGAG GCCGUUAGGC CGAA AUGGUGAG | 7864 |
| 2067 | ACGGCACU C AGGCAAGC | 465 | GCUUGCCU CUGAUGAG GCCGUUAGGC CGAA AGUGCCGU | 7865 |
| 2077 | GGCAAGCU A UUCUGUGU | 466 | ACACAGAA CUGAUGAG GCCGUUAGGC CGAA AGCUUGCC | 7866 |
| 2079 | CAAGCUAU U CUGUGUUG | 467 | CAACACAG CUGAUGAG GCCGUUAGGC CGAA AUAGCUUG | 7867 |
| 2080 | AAGCUAUU C UGUGUUGG | 468 | CCAACACA CUGAUGAG GCCGUUAGGC CGAA AAUAGCUU | 7868 |
| 2086 | UUCUGUGU U GGGGUGAG | 469 | CUCACCCC CUGAUGAG GCCGUUAGGC CGAA UCACAGAA | 7869 |
| 2096 | GGGUGAGU U GAUGAAUC | 470 | GAUUCAUC CUGAUGAG GCCGUUAGGC CGAA ACUCACCC | 7870 |
| 2104 | UGAUGAAU C UAGCCACC | 471 | GGUGGCUA CUGAUGAG GCCGUUAGGC CGAA AUUCAUCA | 7871 |
| 2106 | AUGAAUCU A GCCACCUG | 472 | CAGGUGGC CUGAUGAG GCCGUUAGGC CGAA AGAUUCAU | 7872 |
| 2125 | UGGGAAGU A AUUUGGAA | 473 | UUCCAAAU CUGAUGAG GCCGUUAGGC CGAA ACUUCCCA | 7873 |
| 2128 | GAAGUAAU U UGGAAGAU | 474 | AUCUUCCA CUGAUGAG GCCGUUAGGC CGAA AUUACUUC | 7874 |
| 2129 | AAGUAAUU U GGAAGAUC | 475 | GAUCUUCC CUGAUGAG GCCGUUAGGC CGAA AAUUACUU | 7875 |
| 2137 | UGGAAGAU C CAGCAUCC | 476 | GGAUGCUG CUGAUGAG GCCGUUAGGC CGAA AUCUUCCA | 7876 |
| 2144 | UCCAGCAU C CAGGGAAU | 477 | AUUCCCUG CUGAUGAG GCCGUUAGGC CGAA AUGCUGGA | 7877 |
| 2153 | CAGGGAAU U AGUAGUCA | 478 | UGACUACU CUGAUGAG GCCGUUAGGC CGAA AUUCCCUG | 7878 |
| 2154 | AGGGAAUU A GUAGUCAG | 479 | CUGACUAC CUGAUGAG GCCGUUAGGC CGAA AAUUCCCU | 7879 |
| 2157 | GAAUUAGU A GUCAGCUA | 480 | UAGCUGAC CUGAUGAG GCCGUUAGGC CGAA ACUAAUUC | 7880 |
| 2160 | UUAGUAGU C AGCUAUGU | 481 | ACAUAGCU CUGAUGAG GCCGUUAGGC CGAA ACUACUAA | 7881 |
| 2165 | AGUCAGCU A UGUCAACG | 482 | CGUUGACA CUGAUGAG GCCGUUAGGC CGAA AGCUGACU | 7882 |
| 2169 | AGCUAUGU C AACGUUAA | 483 | UUAACGUU CUGAUGAG GCCGUUAGGC CGAA ACAUAGCU | 7883 |
| 2175 | GUCAACGU U AAUAUGGG | 484 | CCCAUAUU CUGAUGAG GCCGUUAGGC CGAA ACGUUGAC | 7884 |
| 2176 | UCAACGUU A AUAUGGGC | 485 | GCCCAUAU CUGAUGAG GCCGUUAGGC CGAA AACGUUGA | 7885 |
| 2179 | ACGUUAAU A UGGGCCUA | 486 | UAGGCCCA CUGAUGAG GCCGUUAGGC CGAA AUUAACGU | 7886 |
| 2187 | AUGGGCCU A AAAAUCAG | 487 | CUGAUUUU CUGAUGAG GCCGUUAGGC CGAA AGGCCCAU | 7887 |
| 2193 | CUAAAAAU C AGACAACU | 488 | AGUUGUCU CUGAUGAG GCCGUUAGGC CGAA AUUUUUAG | 7888 |
| 2202 | AGACAACU A UUGUGGUU | 489 | AACCACAA CUGAUGAG GCCGUUAGGC CGAA AGUUGUCU | 7889 |
| 2204 | ACAACUAU U GUGGUUUC | 490 | GAAACCAC CUGAUGAG GCCGUUAGGC CGAA AUAGUUGU | 7890 |
| 2210 | AUUGUGGU U UCACAUUU | 491 | AAAUGUGA CUGAUGAG GCCGUUAGGC CGAA ACCACAAU | 7891 |
| 2211 | UUGUGGUU U CACAUUUC | 492 | GAAAUGUG CUGAUGAG GCCGUUAGGC CGAA AACCACAA | 7892 |
| 2212 | UGUGGUUU C ACAUUUCC | 493 | GGAAAUGU CUGAUGAG GCCGUUAGGC CGAA AAACCACA | 7893 |
| 2217 | UUUCACAU U UCCUGUCU | 494 | AGACAGGA CUGAUGAG GCCGUUAGGC CGAA AUGUGAAA | 7894 |
| 2218 | UUCACAUU U CCUGUCUU | 495 | AAGACAGG CUGAUGAG GCCGUUAGGC CGAA AAUGUGAA | 7895 |
| 2219 | UCACAUUU C CUGUCUUA | 496 | UAAGACAG CUGAUGAG GCCGUUAGGC CGAA AAAUGUGA | 7896 |
| 2224 | UUUCCUGU C UUACUUUU | 497 | AAAGUAAU CUGAUGAG GCCGUUAGGC CGAA ACAGGAAA | 7897 |
| 2226 | UCCUGUCU U ACUUUUGG | 498 | CCAAAAGU CUGAUGAG GCCGUUAGGC CGAA AGACAGGA | 7898 |
| 2227 | CCUGUCUU A CUUUUGGG | 499 | CCCAAAAG CUGAUGAG GCCGUUAGGC CGAA AAGACAGG | 7899 |
| 2230 | GUCUUACU U UUGGGCGA | 500 | UCGCCCAA CUGAUGAG GCCGUUAGGC CGAA AGUAAGAC | 7900 |
| 2231 | UCUUACUU U UGGGCGAG | 501 | CUCGCCCA CUGAUGAG GCCGUUAGGC CGAA AAGUAAGA | 7901 |
| 2232 | CUUACUUU U GGGCGAGA | 502 | UCUCGCCC CUGAUGAG GCCGUUAGGC CGAA AAAGUAAG | 7902 |
| 2247 | GAAACUGU U CUUGAAUA | 503 | UAUUCAAG CUGAUGAG GCCGUUAGGC CGAA ACAGUUUC | 7903 |
| 2248 | AAACUGUU C UUGAAUAU | 504 | AUAUUCAA CUGAUGAG GCCGUUAGGC CGAA AACAGUUU | 7904 |
| 2250 | ACUGUUCU U GAAUAUUU | 505 | AAAUAUUC CUGAUGAG GCCGUUAGGC CGAA AGAACAGU | 7905 |
| 2255 | UCUUGAAU A UUUGGUGU | 506 | ACACCAAA CUGAUGAG GCCGUUAGGC CGAA AUUCAAGA | 7906 |
| 2257 | UUGAAUAU U UGGUGUCU | 507 | AGACACCA CUGAUGAG GCCGUUAGGC CGAA AUAUUCAA | 7907 |
| 2258 | UGAAUAUU U GGUGUCUU | 508 | AAGACACC CUGAUGAG GCCGUUAGGC CGAA AAUAUUCA | 7908 |
| 2264 | UUUGGUGU C UUUUGGAG | 509 | CUCCAAAA CUGAUGAG GCCGUUAGGC CGAA ACACCAAA | 7909 |
| 2266 | UGGUGUCU U UUGGAGUG | 510 | CACUCCAA CUGAUGAG GCCGUUAGGC CGAA AGACACCA | 7910 |
| 2267 | GGUGUCUU U UGGAGUGU | 511 | ACACUCCA CUGAUGAG GCCGUUAGGC CGAA AAGACACC | 7911 |
| 2268 | GUGUCUUU U GGAGUGUG | 512 | CACACUCC CUGAUGAG GCCGUUAGGC CGAA AAAGACAC | 7912 |
| 2280 | GUGUGGAU U CGCACUCC | 513 | GGAGUGCG CUGAUGAG GCCGUUAGGC CGAA AUCCACAC | 7913 |
| 2281 | UGUGGAUU C GCACUCCU | 514 | AGGAGUGC CUGAUGAG GCCGUUAGGC CGAA AAUCCACA | 7914 |
| 2287 | UUCGCACU C CUCCUGCA | 515 | UCCAGGAG CUGAUGAG GCCGUUAGGC CGAA AGUGCGAA | 7915 |
| 2290 | GCACUCCU C CUGCAUAU | 516 | AUAUGCAG CUGAUGAG GCCGUUAGGC CGAA AGGAGUGC | 7916 |
| 2297 | UCCUGCAU A UAGACCAC | 517 | GUGGUCUA CUGAUGAG GCCGUUAGGC CGAA AUGCAGGA | 7917 |
| 2299 | CUGCAUAU A GACCACCA | 518 | UGGUGGUC CUGAUGAG GCCGUUAGGC CGAA AUAUGCAG | 7918 |
| 2317 | AUGCCCCU A UCUUAUCA | 519 | UGAUAAGA CUGAUGAG GCCGUUAGGC CGAA AGGGGCAU | 7919 |
| 2319 | GCCCCUAU C UUAUCAAC | 520 | GUUGAUAA CUGAUGAG GCCGUUAGGC CGAA AUAGGGGC | 7920 |
| 2321 | CCCUAUCU U AUCAACAC | 521 | GUGUUGAU CUGAUGAG GCCGUUAGGC CGAA AGAUAGGG | 7921 |
| 2322 | CCUAUCUU A UCAACACU | 522 | AGUGUUGA CUGAUGAG GCCGUUAGGC CGAA AAGAUAGG | 7922 |
| 2324 | UAUCUUAU C AACACUUC | 523 | GAAGUGUU CUGAUGAG GCCGUUAGGC CGAA AUAAGAUA | 7923 |
| 2331 | UCAACACU U CCGGAAAC | 524 | GUUUCCGG CUGAUGAG GCCGUUAGGC CGAA AGUGUUGA | 7924 |
| 2332 | CAACACUU C CGGAAACU | 525 | AGUUUCCG CUGAUGAG GCCGUUAGGC CGAA AAGUGUUG | 7925 |
| 2341 | CGGAAACU A CUGUUGUU | 526 | AACAACAG CUGAUGAG GCCGUUAGGC CGAA AGUUUCCG | 7926 |
| 2346 | ACUACUGU U GUUAGACG | 527 | CGUCUAAC CUGAUGAG GCCGUUAGGC CGAA ACAGUAGU | 7927 |
| 2349 | ACUGUUGU U AGACGAAG | 528 | CUUCGUCU CUGAUGAG GCCGUUAGGC CGAA ACAACAGU | 7928 |
| 2350 | CUGUUGUU A GACGAAGA | 529 | UCUUCGUC CUGAUGAG GCCGUUAGGC CGAA AACAACAG | 7929 |
| 2366 | AGGCAGGU C CCCUAGAA | 530 | UUCUAGGG CUGAUGAG GCCGUUAGGC CGAA ACCUGCCU | 7930 |
| 2371 | GGUCCCCU A GAAGAAGA | 531 | UCUUCUUC CUGAUGAG GCCGUUAGGC CGAA AGGGGACC | 7931 |
| 2383 | GAAGAACU C CCUCGCCU | 532 | AGGCGAGG CUGAUGAG GCCGUUAGGC CGAA AGUUCUUC | 7932 |
| 2387 | AACUCCCU C GCCUCGCA | 533 | UGCGAGGC CUGAUGAG GCCGUUAGGC CGAA AGGGAGUU | 7933 |
| 2392 | CCUCGCCU C GCAGACGA | 534 | UCGUCUGC CUGAUGAG GCCGUUAGGC CGAA AGGCGAGG | 7934 |
| 2405 | ACGAAGGU C UCAAUCGC | 535 | GCGAUUGA CUGAUGAG GCCGUUAGGC CGAA ACCUUCGU | 7935 |
| 2407 | GAAGGUCU C AAUCGCCG | 536 | CGGCGAUU CUGAUGAG GCCGUUAGGC CGAA AGACCUUC | 7936 |
| 2411 | GUCUCAAU C GCCGCGUC | 537 | GACGCGGC CUGAUGAG GCCGUUAGGC CGAA AUUGAGAC | 7937 |
| 2419 | CGCCGCGU C GCAGAAGA | 538 | UCUUCUGC CUGAUGAG GCCGUUAGGC CGAA ACGCGGCG | 7938 |
| 2429 | CAGAAGAU C UCAAUCUC | 539 | GAGAUUGA CUGAUGAG GCCGUUAGGC CGAA AUCUUCUG | 7939 |
| 2431 | GAAGAUCU C AAUCUCGG | 540 | CCGAGAUU CUGAUGAG GCCGUUAGGC CGAA AGAUCUUC | 7940 |
| 2435 | AUCUCAAU C UCGGGAAU | 541 | AUUCCCGA CUGAUGAG GCCGUUAGGC CGAA AUUGAGAU | 7941 |
| 2437 | CUCAAUCU C GGGAAUCU | 542 | AGAUUCCC CUGAUGAG GCCGUUAGGC CGAA AGAUUGAG | 7942 |
| 2444 | UCGGGAAU C UCAAUGUU | 543 | AACAUUGA CUGAUGAG GCCGUUAGGC CGAA AUUCCCGA | 7943 |
| 2446 | GGGAAUCU C AAUGUUAG | 544 | CUAACAUU CUGAUGAG GCCGUUAGGC CGAA AGAUUCCC | 7944 |
| 2452 | CUCAAUGU U AGUAUUCC | 545 | GGAAUACU CUGAUGAG GCCGUUAGGC CGAA ACAUUGAG | 7945 |
| 2453 | UCAAUGUU A GUAUUCCU | 546 | AGGAAUAC CUGAUGAG GCCGUUAGGC CGAA AACAUUGA | 7946 |
| 2456 | AUGUUAGU A UUCCUUGG | 547 | CCAAGGAA CUGAUGAG GCCGUUAGGC CGAA ACUAACAU | 7947 |
| 2458 | GUUAGUAU U CCUUGGAC | 548 | GUCCAAGG CUGAUGAG GCCGUUAGGC CGAA AUACUAAC | 7948 |
| 2459 | UUAGUAUU C CUUGGACA | 549 | UGUCCAAG CUGAUGAG GCCGUUAGGC CGAA AAUACUAA | 7949 |
| 2462 | GUAUUCCU U GGACACAU | 550 | AUGUGUCC CUGAUGAG GCCGUUAGGC CGAA AGGAAUAC | 7950 |
| 2471 | GGACACAU A AGGUGGGA | 551 | UCCCACCU CUGAUGAG GCCGUUAGGC CGAA AUGUGUCC | 7951 |
| 2484 | GGGAAACU U UACGGGGC | 552 | GCCCCGUA CUGAUGAG GCCGUUAGGC CGAA AGUUUCCC | 7952 |
| 2485 | GGAAACUU U ACGGGGCU | 553 | AGCCCCGU CUGAUGAG GCCGUUAGGC CGAA AAGUUUCC | 7953 |
| 2486 | GAAACUUU A CGGGGCUU | 554 | AAGCCCCG CUGAUGAG GCCGUUAGGC CGAA AAAGUUUC | 7954 |
| 2494 | ACGGGGCU U UAUUCUUC | 555 | GAAGAAUA CUGAUGAG GCCGUUAGGC CGAA AGCCCCGU | 7955 |
| 2495 | CGGGGCUU U AUUCUUCU | 556 | AGAAGAAU CUGAUGAG GCCGUUAGGC CGAA AAGCCCCG | 7956 |
| 2496 | GGGGCUUU A UUCUUCUA | 557 | UAGAAGAA CUGAUGAG GCCGUUAGGC CGAA AAAGCCCC | 7957 |
| 2498 | GGCUUUAU U CUUCUACG | 558 | CGUAGAAG CUGAUGAG GCCGUUAGGC CGAA AUAAAGCC | 7958 |
| 2499 | GCUUUAUU C UUCUACGG | 559 | CCGUAGAA CUGAUGAG GCCGUUAGGC CGAA AAUAAAGC | 7959 |
| 2501 | UUUAUUCU U CUACGGUA | 560 | UACCGUAG CUGAUGAG GCCGUUAGGC CGAA AGAAUAAA | 7960 |
| 2502 | UUAUUCUU C UACGGUAC | 561 | GUACCGUA CUGAUGAG GCCGUUAGGC CGAA AAGAAUAA | 7961 |
| 2504 | AUUCUUCU A CGGUACCU | 562 | AGGUACCG CUGAUGAG GCCGUUAGGC CGAA AGAAGAAU | 7962 |
| 2509 | UCUACGGU A CCUUGCUU | 563 | AAGCAAGG CUGAUGAG GCCGUUAGGC CGAA ACCGUAGA | 7963 |
| 2513 | CGGUACCU U GCUUUAAU | 564 | AUUAAAGC CUGAUGAG GCCGUUAGGC CGAA AGGUACCG | 7964 |
| 2517 | ACCUUGCU U UAAUCCUA | 565 | UAGGAUUA CUGAUGAG GCCGUUAGGC CGAA AGCAAGGU | 7965 |
| 2518 | CCUUGCUU U AAUCCUAA | 566 | UUAGGAUU CUGAUGAG GCCGUUAGGC CGAA AAGCAAGG | 7966 |
| 2519 | CUUGCUUU A AUCCUAAA | 567 | UUUAGGAU CUGAUGAG GCCGUUAGGC CGAA AAAGCAAG | 7967 |
| 2522 | GCUUUAAU C CUAAAUGG | 568 | CCAUUUAG CUGAUGAG GCCGUUAGGC CGAA AUUAAAGC | 7968 |
| 2525 | UUAAUCCU A AAUGGCAA | 569 | UUGCCAUU CUGAUGAG GCCGUUAGGC CGAA AGGAUUAA | 7969 |
| 2537 | GGCAAACU C CUUCUUUU | 570 | AAAAGAAG CUGAUGAG GCCGUUAGGC CGAA AGUUUGCC | 7970 |
| 2540 | AAACUCCU U CUUUUCCU | 571 | AGGAAAAG CUGAUGAG GCCGUUAGGC CGAA AGGAGUUU | 7971 |
| 2541 | AACUCCUU C UUUUCCUG | 572 | CAGGAAAA CUGAUGAG GCCGUUAGGC CGAA AAGGAGUU | 7972 |
| 2543 | CUCCUUCU U UUCCUGAC | 573 | GUCAGGAA CUGAUGAG GCCGUUAGGC CGAA AGAAGGAG | 7973 |
| 2544 | UCCUUCUU U UCCUGACA | 574 | UGUCAGGA CUGAUGAG GCCGUUAGGC CGAA AAGAAGGA | 7974 |
| 2545 | CCUUCUUU U CCUGACAU | 575 | AUGUCAGG CUGAUGAG GCCGUUAGGC CGAA AAAGAAGG | 7975 |
| 2546 | CUUCUUUU C CUGACAUU | 576 | AAUGUCAG CUGAUGAG GCCGUUAGGC CGAA AAAAGAAG | 7976 |
| 2554 | CCUGACAU U CAUUUGCA | 577 | UGCAAAUG CUGAUGAG GCCGUUAGGC CGAA AUGUCAGG | 7977 |
| 2555 | CUGACAUU C AUUUGCAG | 578 | CUGCAAAU CUGAUGAG GCCGUUAGGC CGAA AAUGUCAG | 7978 |
| 2558 | ACAUUCAU U UGCAGGAG | 579 | CUCCUGCA CUGAUGAG GCCGUUAGGC CGAA AUGAAUGU | 7979 |
| 2559 | CAUUCAUU U GCAGGAGG | 580 | CCUCCUGC CUGAUGAG GCCGUUAGGC CGAA AAUGAAUG | 7980 |
| 2572 | GAGGACAU U GUUGAUAG | 581 | CUAUCAAC CUGAUGAG GCCGUUAGGC CGAA AUGUCCUC | 7981 |
| 2575 | GACAUUGU U GAUAGAUG | 582 | CAUCUAUC CUGAUGAG GCCGUUAGGC CGAA ACAAUGUC | 7982 |
| 2579 | UUGUUGAU A GAUGUAAG | 583 | CUUACAUC CUGAUGAG GCCGUUAGGC CGAA AUCAACAA | 7983 |
| 2585 | AUAGAUGU A AGCAAUUU | 584 | AAAUUGCU CUGAUGAG GCCGUUAGGC CGAA ACAUCUAU | 7984 |
| 2592 | UAAGCAAU U UGUGGGGC | 585 | GCCCCACA CUGAUGAG GCCGUUAGGC CGAA AUUGCUUA | 7985 |
| 2593 | AAGCAAUU U GUGGGGCC | 586 | GGCCCCAC CUGAUGAG GCCGUUAGGC CGAA AAUUGCUU | 7986 |
| 2605 | GGGCCCCU U ACAGUAAA | 587 | UUUACUGU CUGAUGAG GCCGUUAGGC CGAA AGGGGCCC | 7987 |
| 2606 | GGCCCCUU A CAGUAAAU | 588 | AUUUACUG CUGAUGAG GCCGUUAGGC CGAA AAGGGGCC | 7988 |
| 2611 | CUUACAGU A AAUGAAAA | 589 | UUUUCAUU CUGAUGAG GCCGUUAGGC CGAA ACUGUAAG | 7989 |
| 2629 | AGGAGACU U AAAUUAAC | 590 | GUUAAUUU CUGAUGAG GCCGUUAGGC CGAA AGUCUCCU | 7990 |
| 2630 | GGAGACUU A AAUUAACU | 591 | AGUUAAUU CUGAUGAG GCCGUUAGGC CGAA AAGUCUCC | 7991 |
| 2634 | ACUUAAAU U AACUAUGC | 592 | GCAUAGUU CUGAUGAG GCCGUUAGGC CGAA AUUUAAGU | 7992 |
| 2635 | CUUAAAUU A ACUAUGCC | 593 | GGCAUAGU CUGAUGAG GCCGUUAGGC CGAA AAUUUAAG | 7993 |
| 2639 | AAUUAACU A UGCCUGCU | 594 | AGCAGGCA CUGAUGAG GCCGUUAGGC CGAA AGUUAAUU | 7994 |
| 2648 | UGCCUGCU A GGUUUUAU | 595 | AUAAAACC CUGAUGAG GCCGUUAGGC CGAA AGCAGGCA | 7995 |
| 2652 | UGCUAGGU U UUAUCCCA | 596 | UGGGAUAA CUGAUGAG GCCGUUAGGC CGAA ACCUAGCA | 7996 |
| 2653 | GCUAGGUU U UAUCCCAA | 597 | UUGGGAUA CUGAUGAG GCCGUUAGGC CGAA AACCUAGC | 7997 |
| 2654 | CUAGGUUU U AUCCCAAU | 598 | AUUGGGAU CUGAUGAG GCCGUUAGGC CGAA AAACCUAG | 7998 |
| 2655 | UAGGUUUU A UCCCAAUG | 599 | CAUUGGGA CUGAUGAG GCCGUUAGGC CGAA AAAACCUA | 7999 |
| 2657 | GGUUUUAU C CCAAUGUU | 600 | AACAUUGG CUGAUGAG GCCGUUAGGC CGAA AUAAAACC | 8000 |
| 2665 | CCCAAUGU U ACUAAAUA | 601 | UAUUUAGU CUGAUGAG GCCGUUAGGC CGAA ACAUUGGG | 8001 |
| 2666 | CCAAUGUU A CUAAAUAU | 602 | AUAUUUAG CUGAUGAG GCCGUUAGGC CGAA AACAUUGG | 8002 |
| 2669 | AUGUUACU A AAUAUUUG | 603 | CAAAUAUU CUGAUGAG GCCGUUAGGC CGAA AGUAACAU | 8003 |
| 2673 | UACUAAAU A UUUGCCCU | 604 | AGGGCAAA CUGAUGAG GCCGUUAGGC CGAA AUUUAGUA | 8004 |
| 2675 | CUAAAUAU U UGCCCUUA | 605 | UAAGGGCA CUGAUGAG GCCGUUAGGC CGAA AUAUUUAG | 8005 |
| 2676 | UAAAUAUU U GCCCUUAG | 606 | CUAAGGGC CUGAUGAG GCCGUUAGGC CGAA AAUAUUUA | 8006 |
| 2682 | UUUGCCCU U AGAUAAAG | 607 | CUUUAUCU CUGAUGAG GCCGUUAGGC CGAA AGGGCAAA | 8007 |
| 2683 | UUGCCCUU A GAUAAAGG | 608 | CCUUUAUC CUGAUGAG GCCGUUAGGC CGAA AAGGGCAA | 8008 |
| 2687 | CCUUAGAU A AAGGGAUC | 609 | GAUCCCUU CUGAUGAG GCCGUUAGGC CGAA AUCUAAGG | 8009 |
| 2695 | AAAGGGAU C AAACCGUA | 610 | UACGGUUU CUGAUGAG GCCGUUAGGC CGAA AUCCCUUU | 8010 |
| 2703 | CAAACCGU A UUAUCCAG | 611 | CUGGAUAA CUGAUGAG GCCGUUAGGC CGAA ACGGUUUG | 8011 |
| 2705 | AACCGUAU U AUCCAGAG | 612 | CUCUGGAU CUGAUGAG GCCGUUAGGC CGAA AUACGGUU | 8012 |
| 2706 | ACCGUAUU A UCCAGAGU | 613 | ACUCUGGA CUGAUGAG GCCGUUAGGC CGAA AAUACGGU | 8013 |
| 2708 | CGUAUUAU C CAGAGUAU | 614 | AUACUCUG CUGAUGAG GCCGUUAGGC CGAA AUAAUACG | 8014 |
| 2715 | UCCAGAGU A UGUAGUUA | 615 | UAACUACA CUGAUGAG GCCGUUAGGC CGAA ACUCUGGA | 8015 |
| 2719 | GAGUAUGU A GUUAAUCA | 616 | UGAUUAAC CUGAUGAG GCCGUUAGGC CGAA ACAUACUC | 8016 |
| 2722 | UAUGUAGU U AAUCAUUA | 617 | UAAUGAUU CUGAUGAG GCCGUUAGGC CGAA ACUACAUA | 8017 |
| 2723 | AUGUAGUU A AUCAUUAC | 618 | GUAAUGAU CUGAUGAG GCCGUUAGGC CGAA AACUACAU | 8018 |
| 2726 | UAGUUAAU C AUUACUUC | 619 | GAAGUAAU CUGAUGAG GCCGUUAGGC CGAA AUUAACUA | 8019 |
| 2729 | UUAAUCAU U ACUUCCAG | 620 | CUGGAAGU CUGAUGAG GCCGUUAGGC CGAA AUGAUUAA | 8020 |
| 2730 | UAAUCAUU A CUUCCAGA | 621 | UCUGGAAG CUGAUGAG GCCGUUAGGC CGAA AAUGAUUA | 8021 |
| 2733 | UCAUUACU U CCAGACGC | 622 | GCGUCUGG CUGAUGAG GCCGUUAGGC CGAA AGUAAUGA | 8022 |
| 2734 | CAUUACUU C CAGACGCG | 623 | CGCGUCUG CUGAUGAG GCCGUUAGGC CGAA AAGUAAUG | 8023 |
| 2747 | CGCGACAU U AUUUACAC | 624 | GUGUAAAU CUGAUGAG GCCGUUAGGC CGAA AUGUCGCG | 8024 |
| 2748 | GCGACAUU A UUUACACA | 625 | UGUGUAAA CUGAUGAG GCCGUUAGGC CGAA AAUGUCGC | 8025 |
| 2750 | GACAUUAU U UACACACU | 626 | AGUGUGUA CUGAUGAG GCCGUUAGGC CGAA AUAAUGUC | 8026 |
| 2751 | ACAUUAUU U ACACACUC | 627 | GAGUGUGU CUGAUGAG GCCGUUAGGC CGAA AAUAAUGU | 8027 |
| 2752 | CAUUAUUU A CACACUCU | 628 | AGAGUGUG CUGAUGAG GCCGUUAGGC CGAA AAAUAAUG | 8028 |
| 2759 | UACACACU C UUUGGAAG | 629 | CUUCCAAA CUGAUGAG GCCGUUAGGC CGAA AGUGUGUA | 8029 |
| 2761 | CACACUCU U UGGAAGGC | 630 | GCCUUCCA CUGAUGAG GCCGUUAGGC CGAA AGAGUGUG | 8030 |
| 2762 | ACACUCUU U GGAAGGCG | 631 | CGCCUUCC CUGAUGAG GCCGUUAGGC CGAA AAGAGUGU | 8031 |
| 2776 | GCGGGGAU C UUAUAUAA | 632 | UUAUAUAA CUGAUGAG GCCGUUAGGC CGAA AUCCCCGC | 8032 |
| 2778 | GGGGAUCU U AUAUAAAA | 633 | UUUUAUAU CUGAUGAG GCCGUUAGGC CGAA AGAUCCCC | 8033 |
| 2779 | GGGAUCUU A UAUAAAAG | 634 | CUUUUAUA CUGAUGAG GCCGUUAGGC CGAA AAGAUCCC | 8034 |
| 2781 | GAUCUUAU A UAAAAGAG | 635 | CUCUUUUA CUGAUGAG GCCGUUAGGC CGAA AUAAGAUC | 8035 |
| 2783 | UCUUAUAU A AAAGAGAG | 636 | CUCUCUUU CUGAUGAG GCCGUUAGGC CGAA AUAUAAGA | 8036 |
| 2793 | AAGAGAGU C CACACGUA | 637 | UACGUGUG CUGAUGAG GCCGUUAGGC CGAA ACUCUCUU | 8037 |
| 2801 | CCACACGU A GCGCCUCA | 638 | UGAGGCGC CUGAUGAG GCCGUUAGGC CGAA ACGUGUGG | 8038 |
| 2808 | UAGCGCCU C AUUUUGCG | 639 | CGCAAAAU CUGAUGAG GCCGUUAGGC CGAA AGGCGCUA | 8039 |
| 2811 | CGCCUCAU U UUGCGGGU | 640 | ACCCGCAA CUGAUGAG GCCGUUAGGC CGAA AUGAGGCG | 8040 |
| 2812 | GCCUCAUU U UGCGGGUC | 641 | GACCCGCA CUGAUGAG GCCGUUAGGC CGAA AAUGAGGC | 8041 |
| 2813 | CCUCAUUU U GCGGGUCA | 642 | UGACCCGC CUGAUGAG GCCGUUAGGC CGAA AAAUGAGG | 8042 |
| 2820 | UUGCGGGU C ACCAUAUU | 643 | AAUAUGGU CUGAUGAG GCCGUUAGGC CGAA ACCCGCAA | 8043 |
| 2826 | GUCACCAU A UUCUUGGG | 644 | CCCAAGAA CUGAUGAG GCCGUUAGGC CGAA AUGGUGAC | 8044 |
| 2828 | CACCAUAU U CUUGGGAA | 645 | UUCCCAAG CUGAUGAG GCCGUUAGGC CGAA AUAUGGUG | 8045 |
| 2829 | ACCAUAUU C UUGGGAAC | 646 | GUUCCCAA CUGAUGAG GCCGUUAGGC CGAA AAUAUGGU | 8046 |
| 2831 | CAUAUUCU U GGGAACAA | 647 | UUGUUCCC CUGAUGAG GCCGUUAGGC CGAA AGAAUAUG | 8047 |
| 2843 | AACAAGAU C UACAGCAU | 648 | AUGCUGUA CUGAUGAG GCCGUUAGGC CGAA AUCUUGUU | 8048 |
| 2845 | CAAGAUCU A CAGCAUGG | 649 | CCAUGCUG CUGAUGAG GCCGUUAGGC CGAA AGAUCUUG | 8049 |
| 2859 | UGGGAGGU U GGUCUUCC | 650 | GGAAGACC CUGAUGAG GCCGUUAGGC CGAA ACCUCCCA | 8050 |
| 2863 | AGGUUGGU C UUCCAAAC | 651 | GUUUGGAA CUGAUGAG GCCGUUAGGC CGAA ACCAACCU | 8051 |
| 2865 | GUUGGUCU U CCAAACCU | 652 | AGGUUUGG CUGAUGAG GCCGUUAGGC CGAA AGACCAAC | 8052 |
| 2866 | UUGGUCUU C CAAACCUC | 653 | GAGGUUUG CUGAUGAG GCCGUUAGGC CGAA AAGACCAA | 8053 |
| 2874 | CCAAACCU C GAAAAGGC | 654 | GCCUUUUC CUGAUGAG GCCGUUAGGC CGAA AGGUUUGG | 8054 |
| 2895 | GGACAAAU C UUUCUGUC | 655 | GACAGAAA CUGAUGAG GCCGUUAGGC CGAA AUUUGUCC | 8055 |
| 2897 | ACAAAUCU U UCUGUCCC | 656 | GGGACAGA CUGAUGAG GCCGUUAGGC CGAA AGAUUUGU | 8056 |
| 2898 | CAAAUCUU U CUGUCCCC | 657 | GGGGACAG CUGAUGAG GCCGUUAGGC CGAA AAGAUUUG | 8057 |
| 2899 | AAAUCUUU C UGUCCCCA | 658 | UGGGGACA CUGAUGAG GCCGUUAGGC CGAA AAAGAUUU | 8058 |
| 2903 | CUUUCUGU C CCCAAUCC | 659 | GGAUUGGG CUGAUGAG GCCGUUAGGC CGAA ACAGAAAG | 8059 |
| 2910 | UCCCCAAU C CCCUGGGA | 660 | UCCCAGGG CUGAUGAG GCCGUUAGGC CGAA AUUGGGGA | 8060 |
| 2920 | CCUGGGAU U CUUCCCCG | 661 | CGGGGAAG CUGAUGAG GCCGUUAGGC CGAA AUCCCAGG | 8061 |
| 2921 | CUGGGAUU C UUCCCCGA | 662 | UCGGGGAA CUGAUGAG GCCGUUAGGC CGAA AAUCCCAG | 8062 |
| 2923 | GGGAUUCU U CCCCGAUC | 663 | GAUCGGGG CUGAUGAG GCCGUUAGGC CGAA AGAAUCCC | 8063 |
| 2924 | GGAUUCUU C CCCGAUCA | 664 | UGAUCGGG CUGAUGAG GCCGUUAGGC CGAA AAGAAUCC | 8064 |
| 2931 | UCCCCGAU C AUCAGUUG | 665 | CAACUGAU CUGAUGAG GCCGUUAGGC CGAA AUCGGGGA | 8065 |
| 2934 | CCGAUCAU C AGUUGGAC | 666 | GUCCAACU CUGAUGAG GCCGUUAGGC CGAA AUGAUCGG | 8066 |
| 2938 | UCAUCAGU U GGACCCUG | 667 | CAGGGUCC CUGAUGAG GCCGUUAGGC CGAA ACUGAUGA | 8067 |
| 2950 | CCCUGCAU U CAAAGCCA | 668 | UGGCUUUG CUGAUGAG GCCGUUAGGC CGAA AUGCAGGG | 8068 |
| 2951 | CCUGCAUU C AAAGCCAA | 669 | UUGGCUUU CUGAUGAG GCCGUUAGGC CGAA AAUGCAGG | 8069 |
| 2962 | AGCCAACU C AGUAAAUC | 670 | GAUUUACU CUGAUGAG GCCGUUAGGC CGAA AGUUGGCU | 8070 |
| 2966 | AACUCAGU A AAUCCAGA | 671 | UCUGGAUU CUGAUGAG GCCGUUAGGC CGAA ACUGAGUU | 8071 |
| 2970 | CAGUAAAU C CAGAUUGG | 672 | CCAAUCUG CUGAUGAG GCCGUUAGGC CGAA AUUUACUG | 8072 |
| 2976 | AUCCAGAU U GGGACCUC | 673 | GAGGUCCC CUGAUGAG GCCGUUAGGC CGAA AUCUGGAU | 8073 |
| 2984 | UGGGACCU C AACCCGCA | 674 | UGCGGGUU CUGAUGAG GCCGUUAGGC CGAA AGGUCCCA | 8074 |
| 3037 | GGGAGCAU U CGGGCCAG | 675 | CUGGCCCG CUGAUGAG GCCGUUAGGC CGAA AUGCUCCC | 8075 |
| 3038 | GGAGCAUU C GGGCCAGG | 676 | CCUGGCCC CUGAUGAG GCCGUUAGGC CGAA AAUGCUCC | 8076 |
| 3049 | GCCAGGGU U CACCCCUC | 677 | GAGGGGUG CUGAUGAG GCCGUUAGGC CGAA ACCCUGGC | 8077 |
| 3050 | CCAGGGUU C ACCCCUCC | 678 | GGAGGGGU CUGAUGAG GCCGUUAGGC CGAA AACCCUGG | 8078 |
| 3057 | UCACCCCU C CCCAUGGG | 679 | CCCAUGGG CUGAUGAG GCCGUUAGGC CGAA AGGGGUGA | 8079 |
| 3073 | GGGACUGU U GGGGUGGA | 680 | UCCACCCC CUGAUGAG GCCGUUAGGC CGAA ACAGUCCC | 8080 |
| 3087 | GGAGCCCU C ACGCUCAG | 681 | CUGAGCGU CUGAUGAG GCCGUUAGGC CGAA AGGGCUCC | 8081 |
| 3093 | CUCACGCU C AGGGCCUA | 682 | UAGGCCCU CUGAUGAG GCCGUUAGGC CGAA AGCGUGAG | 8082 |
| 3101 | CAGGGCCU A CUCACAAC | 683 | GUUGUGAG CUGAUGAG GCCGUUAGGC CGAA AGGCCCUG | 8083 |
| 3104 | GGCCUACU C ACAACUGU | 684 | ACAGUUGU CUGAUGAG GCCGUUAGGC CGAA AGUAGGCC | 8084 |
| 3123 | CAGCAGCU C CUCCUCCU | 685 | AGGAGGAG CUGAUGAG GCCGUUAGGC CGAA AGCUGCUG | 8085 |
| 3126 | CAGCUCCU C CUCCUGCC | 686 | GGCAGGAG CUGAUGAG GCCGUUAGGC CGAA AGGAGCUG | 8086 |
| 3129 | CUCCUCCU C CUGCCUCC | 687 | GGAGGCAG CUGAUGAG GCCGUUAGGC CGAA AGGAGGAG | 8087 |
| 3136 | UCCUGCCU C CACCAAUC | 688 | GAUUGGUG CUGAUGAG GCCGUUAGGC CGAA AGGCAGGA | 8088 |
| 3144 | CCACCAAU C GGCAGUCA | 689 | UGACUGCC CUGAUGAG GCCGUUAGGC CGAA AUUGGUGG | 8089 |
| 3151 | UCGGCAGU C AGGAAGGC | 690 | GCCUUCCU CUGAUGAG GCCGUUAGGC CGAA ACUGCCGA | 8090 |
| 3165 | GGCAGCCU A CUCCCUUA | 691 | UAAGGGAG CUGAUGAG GCCGUUAGGC CGAA AGGCUGCC | 8091 |
| 3168 | AGCCUACU C CCUUAUCU | 692 | AGAUAAGG CUGAUGAG GCCGUUAGGC CGAA AGUAGGCU | 8092 |
| 3172 | UACUCCCU U AUCUCCAC | 693 | GUGGAGAU CUGAUGAG GCCGUUAGGC CGAA AGGGAGUA | 8093 |
| 3173 | ACUCCCUU A UCUCCACC | 694 | GGUGGAGA CUGAUGAG GCCGUUAGGC CGAA AAGGGAGU | 8094 |
| 3175 | UCCCUUAU C UCCACCUC | 695 | GAGGUGGA CUGAUGAG GCCGUUAGGC CGAA AUAAGGGA | 8095 |
| 3177 | CCUUAUCU C CACCUCUA | 696 | UAGAGGUG CUGAUGAG GCCGUUAGGC CGAA AGAUAAGG | 8096 |
| 3183 | CUCCACCU C UAAGGGAC | 697 | GUCCCUUA CUGAUGAG GCCGUUAGGC CGAA AGGUGGAG | 8097 |
| 3185 | CCACCUCU A AGGGACAC | 698 | GUGUCCCU CUGAUGAG GCCGUUAGGC CGAA AGAGGUGG | 8098 |
| 3195 | GGGACACU C AUCCUCAG | 699 | CUGAGGAU CUGAUGAG GCCGUUAGGC CGAA AGUGUCCC | 8099 |
| 3198 | ACACUCAU C CUCAGGCC | 700 | GGCCUGAG CUGAUGAG GCCGUUAGGC CGAA AUGAGUGU | 8100 |
| 3201 | CUCAUCCU C AGGCCAUG | 701 | CAUGGCCU CUGAUGAG GCCGUUAGGC CGAA AGGAUGAG | 8101 |

Input Sequence = AF100308.
Cut Site = UH/.
Stem Length = 8.
Core Sequence = CUGAUGAG GCCGUUAGGC CGAA
AF100308 (Hepatitis B virus strain 2-18, 3215 bp)
Underlined region can be any X sequence or linker, as described herein.

TABLE VI

HUMAN HBV INOZYME AND SUBSTRATE SEQUENCE

| | | | | |
|---|---|---|---|---|
| Pos | Substrate | Seq ID | Inozyme | Seq ID |

| | | | | |
|---|---|---|---|---|
| 9 | AACUCCAC C ACUUUCCA | 702 | UGGAAAGU CUGAUGAG GCCGUUAGGC CGAA IUGGAGUU | 8102 |
| 10 | ACUCCACC A CUUUCCAC | 703 | GUGGAAAG CUGAUGAG GCCGUUAGGC CGAA IGUGGAGU | 8103 |
| 12 | UCCACCAC U UUCCACCA | 704 | UGGUGGAA CUGAUGAG GCCGUUAGGC CGAA IUGGUGGA | 8104 |
| 16 | CCACUUUC C ACCAAACU | 705 | AGUUUGGU CUGAUGAG GCCGUUAGGC CGAA IAAAGUGG | 8105 |
| 17 | CACUUUCC A CCAAACUC | 706 | GAGUUUGG CUGAUGAG GCCGUUAGGC CGAA IGAAAGUG | 8106 |
| 19 | CUUUCCAC C AAACUCUU | 707 | AAGAGUUU CUGAUGAG GCCGUUAGGC CGAA IUGGAAAG | 8107 |
| 20 | UUUCCACC A AACUCUUC | 708 | GAAGAGUU CUGAUGAG GCCGUUAGGC CGAA IGUGGAAA | 8108 |
| 24 | CACCAAAC U CUUCAAGA | 709 | UCUUGAAG CUGAUGAG GCCGUUAGGC CGAA IUUUGGUG | 8109 |
| 26 | CCAAACUC U UCAAGAUC | 710 | GAUCUUGA CUGAUGAG GCCGUUAGGC CGAA IAGUUUGG | 8110 |
| 29 | AACUCUUC A AGAUCCCA | 711 | UGGGAUCU CUGAUGAG GCCGUUAGGC CGAA IAAGAGUU | 8111 |
| 35 | UCAAGAUC C CAGAGUCA | 712 | UGACUCUG CUGAUGAG GCCGUUAGGC CGAA IAUCUUGA | 8112 |
| 36 | CAAGAUCC C AGAGUCAG | 713 | CUGACUCU CUGAUGAG GCCGUUAGGC CGAA IGAUCUUG | 8113 |
| 37 | AAGAUCCC A GAGUCAGG | 714 | CCUGACUC CUGAUGAG GCCGUUAGGC CGAA IGGAUCUU | 8114 |
| 43 | CCAGAGUC A GGGCCCUG | 715 | CAGGGCCC CUGAUGAG GCCGUUAGGC CGAA IACUCUGG | 8115 |
| 48 | GUCAGGGC C CUGUACUU | 716 | AAGUACAG CUGAUGAG GCCGUUAGGC CGAA ICCCUGAC | 8116 |
| 49 | UCAGGGCC C UGUACUUU | 717 | AAAGUACA CUGAUGAG GCCGUUAGGC CGAA IGCCCUGA | 8117 |
| 50 | CAGGGCCC U GUACUUUC | 718 | GAAAGUAC CUGAUGAG GCCGUUAGGC CGAA IGGCCCUG | 8118 |
| 55 | CCCUGUAC U UUCCUGCU | 719 | AGCAGGAA CUGAUGAG GCCGUUAGGC CGAA IUACAGGG | 8119 |
| 59 | GUACUUUC C UGCUGGUG | 720 | CACCAGCA CUGAUGAG GCCGUUAGGC CGAA IAAAGUAC | 8120 |
| 60 | UACUUUCC U GCUGGUGG | 721 | CCACCAGC CUGAUGAG GCCGUUAGGC CGAA IGAAAGUA | 8121 |
| 63 | UUUCCUGC U GGUGGCUC | 722 | GAGCCACC CUGAUGAG GCCGUUAGGC CGAA ICAGGAAA | 8122 |
| 70 | CUGGUGGC U CCAGUUCA | 723 | UGAACUGG CUGAUGAG GCCGUUAGGC CGAA ICCACCAG | 8123 |
| 72 | GGUGGCUC C AGUUCAGG | 724 | CCUGAACU CUGAUGAG GCCGUUAGGC CGAA IAGCCACC | 8124 |
| 73 | GUGGCUCC A GUUCAGGA | 725 | UCCUGAAC CUGAUGAG GCCGUUAGGC CGAA IGAGCCAC | 8125 |
| 78 | UCCAGUUC A GGAACAGU | 726 | ACUGUUCC CUGAUGAG GCCGUUAGGC CGAA IAACUGGA | 8126 |
| 84 | UCAGGAAC A GUGAGCCC | 727 | GGGCUCAC CUGAUGAG GCCGUUAGGC CGAA IUUCCUGA | 8127 |
| 91 | CAGUGAGC C CUGCUCAG | 728 | CUGAGCAG CUGAUGAG GCCGUUAGGC CGAA ICUCACUG | 8128 |
| 92 | AGUGAGCC C UGCUCAGA | 729 | UCUGAGCA CUGAUGAG GCCGUUAGGC CGAA IGCUCACU | 8129 |
| 93 | GUGAGCCC U GCUCAGAA | 730 | UUCUGAGC CUGAUGAG GCCGUUAGGC CGAA IGGCUCAC | 8130 |
| 96 | AGCCCUGC U CAGAAUAC | 731 | GUAUUCUG CUGAUGAG GCCGUUAGGC CGAA ICAGGGCU | 8131 |
| 98 | CCCUGCUC A GAAUACUG | 732 | CAGUAUUC CUGAUGAG GCCGUUAGGC CGAA IAGCAGGG | 8132 |
| 105 | CAGAAUAC U GUCUCUGC | 733 | GCAGAGAC CUGAUGAG GCCGUUAGGC CGAA IUAUUCUG | 8133 |
| 109 | AUACUGUC U CUGCCAUA | 734 | UAUGGCAG CUGAUGAG GCCGUUAGGC CGAA IACAGUAU | 8134 |
| 111 | ACUGUCUC U GCCAUAUC | 735 | GAUAUGGC CUGAUGAG GCCGUUAGGC CGAA IAGACAGU | 8135 |
| 114 | GUCUCUGC C AUAUCGUC | 736 | GACGAUAU CUGAUGAG GCCGUUAGGC CGAA ICAGAGAC | 8136 |
| 115 | UCUCUGCC A UAUCGUCA | 737 | UGACGAUA CUGAUGAG GCCGUUAGGC CGAA IGCAGAGA | 8137 |
| 123 | AUAUCGUC A AUCUUAUC | 738 | GAUAAGAU CUGAUGAG GCCGUUAGGC CGAA IACGAUAU | 8138 |
| 127 | CGUCAAUC U UAUCGAAG | 739 | CUUCGAUA CUGAUGAG GCCGUUAGGC CGAA IAUUGACG | 8139 |
| 138 | UCGAAGAC U GGGGACCC | 740 | GGGUCCCC CUGAUGAG GCCGUUAGGC CGAA IUCUUCGA | 8140 |
| 145 | CUGGGGAC C CUGUACCG | 741 | CGGUACAG CUGAUGAG GCCGUUAGGC CGAA IUCCCCAG | 8141 |
| 146 | UGGGGACC C UGUACCGA | 742 | UCGGUACA CUGAUGAG GCCGUUAGGC CGAA IGUCCCCA | 8142 |
| 147 | GGGGACCC U GUACCGAA | 743 | UUCGGUAC CUGAUGAG GCCGUUAGGC CGAA IGGUCCCC | 8143 |
| 152 | CCCUGUAC C GAACAUGG | 744 | CCAUGUUC CUGAUGAG GCCGUUAGGC CGAA IUACAGGG | 8144 |
| 157 | UACCGAAC A UGGAGAAC | 745 | GUUCUCCA CUGAUGAG GCCGUUAGGC CGAA IUUCGGUA | 8145 |
| 166 | UGGAGAAC A UCGCAUCA | 746 | UGAUGCGA CUGAUGAG GCCGUUAGGC CGAA IUUCUCCA | 8146 |
| 171 | AACAUCGC A UCAGGACU | 747 | AGUCCUGA CUGAUGAG GCCGUUAGGC CGAA ICGAUGUU | 8147 |
| 174 | AUCGCAUC A GGACUCCU | 748 | AGGAGUCC CUGAUGAG GCCGUUAGGC CGAA IAUGCGAU | 8148 |
| 179 | AUCAGGAC U CCUAGGAC | 749 | GUCCUAGG CUGAUGAG GCCGUUAGGC CGAA IUCCUGAU | 8149 |
| 181 | CAGGACUC C UAGGACCC | 750 | GGGUCCUA CUGAUGAG GCCGUUAGGC CGAA IAGUCCUG | 8150 |
| 182 | AGGACUCC U AGGACCCC | 751 | GGGGUCCU CUGAUGAG GCCGUUAGGC CGAA IGAGUCCU | 8151 |
| 188 | CCUAGGAC C CCUGCUCG | 752 | CGAGCAGG CUGAUGAG GCCGUUAGGC CGAA IUCCUAGG | 8152 |
| 189 | CUAGGACC C CUGCUCGU | 753 | ACGAGCAG CUGAUGAG GCCGUUAGGC CGAA IGUCCUAG | 8153 |
| 190 | UAGGACCC C UGCUCGUG | 754 | CACGAGCA CUGAUGAG GCCGUUAGGC CGAA IGGUCCUA | 8154 |
| 191 | AGGACCCC U GCUCGUGU | 755 | ACACGAGC CUGAUGAG GCCGUUAGGC CGAA IGGGUCCU | 8155 |
| 194 | ACCCCUGC U CGUGUUAC | 756 | GUAACACG CUGAUGAG GCCGUUAGGC CGAA ICAGGGGU | 8156 |
| 203 | CGUGUUAC A GGCGGGGU | 757 | ACCCCGCC CUGAUGAG GCCGUUAGGC CGAA IUAACACG | 8157 |
| 217 | GGUUUUUC U UGUUGACA | 758 | UGUCAACA CUGAUGAG GCCGUUAGGC CGAA IAAAAACC | 8158 |
| 225 | UUGUUGAC A AAAAUCCU | 759 | AGGAUUUU CUGAUGAG GCCGUUAGGC CGAA IUCAACAA | 8159 |
| 232 | CAAAAAUC C UCACAAUA | 760 | UAUUGUGA CUGAUGAG GCCGUUAGGC CGAA IAUUUUUG | 8160 |
| 233 | AAAAAUCC U CACAAUAC | 761 | GUAUUGUG CUGAUGAG GCCGUUAGGC CGAA IGAUUUUU | 8161 |
| 235 | AAAUCCUC A CAAUACCA | 762 | UGGUAUUG CUGAUGAG GCCGUUAGGC CGAA IAGGAUUU | 8162 |
| 237 | AUCCUCAC A AUACCACA | 763 | UGUGGUAU CUGAUGAG GCCGUUAGGC CGAA IUGAGGAU | 8163 |
| 242 | CACAAUAC C ACAGAGUC | 764 | GACUCUGU CUGAUGAG GCCGUUAGGC CGAA IUAUUGUG | 8164 |
| 243 | ACAAUACC A CAGAGUCU | 765 | AGACUCUG CUGAUGAG GCCGUUAGGC CGAA IGUAUUGU | 8165 |
| 245 | AAUACCAC A GAGUCUAG | 766 | CUAGACUC CUGAUGAG GCCGUUAGGC CGAA IUGGUAUU | 8166 |
| 251 | ACAGAGUC U AGACUCGU | 767 | ACGAGUCU CUGAUGAG GCCGUUAGGC CGAA IACUCUGU | 8167 |
| 256 | GUCUAGAC U CGUGGUGG | 768 | CCACCACG CUGAUGAG GCCGUUAGGC CGAA IUCUAGAC | 8168 |
| 267 | UGGUGGAC U UCUCUCAA | 769 | UUGAGAGA CUGAUGAG GCCGUUAGGC CGAA IUCCACCA | 8169 |
| 270 | UGGACUUC U CUCAAUUU | 770 | AAAUUGAG CUGAUGAG GCCGUUAGGC CGAA IAAGUCCA | 8170 |
| 272 | GACUUCUC U CAAUUUUC | 771 | GAAAAUUG CUGAUGAG GCCGUUAGGC CGAA IAGAAGUC | 8171 |
| 274 | CUUCUCUC A AUUUUCUA | 772 | UAGAAAAU CUGAUGAG GCCGUUAGGC CGAA IAGAGAAG | 8172 |
| 281 | CAAUUUUC U AGGGGGAA | 773 | UUCCCCCU CUGAUGAG GCCGUUAGGC CGAA IAAAAUUG | 8173 |
| 291 | GGGGGAAC A CCCGUGUG | 774 | CACACGGG CUGAUGAG GCCGUUAGGC CGAA IUUCCCCC | 8174 |
| 293 | GGGAACAC C CGUGUGUC | 775 | GACACACG CUGAUGAG GCCGUUAGGC CGAA IUGUUCCC | 8175 |
| 294 | GGAACACC C GUGUGUCU | 776 | AGACACAC CUGAUGAG GCCGUUAGGC CGAA IGUGUUCC | 8176 |
| 302 | CGUGUGUC U UGGCCAAA | 777 | UUUGGCCA CUGAUGAG GCCGUUAGGC CGAA IACACACG | 8177 |
| 307 | GUCUUGGC C AAAAUUCG | 778 | CGAAUUUU CUGAUGAG GCCGUUAGGC CGAA ICCAAGAC | 8178 |
| 308 | UCUUGGCC A AAAUUCGC | 779 | GCGAAUUU CUGAUGAG GCCGUUAGGC CGAA IGCCAAGA | 8179 |
| 317 | AAAUUCGC A GUCCCAAA | 780 | UUUGGGAC CUGAUGAG GCCGUUAGGC CGAA ICGAAUUU | 8180 |
| 321 | UCGCAGUC C CAAAUCUC | 781 | GAGAUUUG CUGAUGAG GCCGUUAGGC CGAA IACUGCGA | 8181 |
| 322 | CGCAGUCC C AAAUCUCC | 782 | GGAGAUUU CUGAUGAG GCCGUUAGGC CGAA IGACUGCG | 8182 |
| 323 | GCAGUCCC A AAUCUCCA | 783 | UGGAGAUU CUGAUGAG GCCGUUAGGC CGAA IGGACUGC | 8183 |
| 328 | CCCAAAUC U CCAGUCAC | 784 | GUGACUGG CUGAUGAG GCCGUUAGGC CGAA IAUUUGGG | 8184 |
| 330 | CAAAUCUC C AGUCACUC | 785 | GAGUGACU CUGAUGAG GCCGUUAGGC CGAA IAGAUUUG | 8185 |
| 331 | AAAUCUCC A GUCACUCA | 786 | UGAGUGAC CUGAUGAG GCCGUUAGGC CGAA IGAGAUUU | 8186 |
| 335 | CUCCAGUC A CUCACCAA | 787 | UUGGUGAG CUGAUGAG GCCGUUAGGC CGAA IACUGGAG | 8187 |
| 337 | CCAGUCAC U CACCAACC | 788 | GGUUGGUG CUGAUGAG GCCGUUAGGC CGAA IUGACUGG | 8188 |
| 339 | AGUCACUC A CCAACCUG | 789 | CAGGUUGG CUGAUGAG GCCGUUAGGC CGAA IAGUGACU | 8189 |
| 341 | UCACUCAC C AACCUGUU | 790 | AACAGGUU CUGAUGAG GCCGUUAGGC CGAA IUGAGUGA | 8190 |
| 342 | CACUCACC A ACCUGUUG | 791 | CAACAGGU CUGAUGAG GCCGUUAGGC CGAA IGUGAGUG | 8191 |
| 345 | UCACCAAC C UGUUGUCC | 792 | GGACAACA CUGAUGAG GCCGUUAGGC CGAA IUUGGUGA | 8192 |
| 346 | CACCAACC U GUUGUCCU | 793 | AGGACAAC CUGAUGAG GCCGUUAGGC CGAA IGUUGGUG | 8193 |
| 353 | CUGUUGUC C UCCAAUUU | 794 | AAAUUGGA CUGAUGAG GCCGUUAGGC CGAA IACAACAG | 8194 |
| 354 | UGUUGUCC U CCAAUUUG | 795 | CAAAUUGG CUGAUGAG GCCGUUAGGC CGAA IGACAACA | 8195 |
| 356 | UUGUCCUC C AAUUUGUC | 796 | GACAAAUU CUGAUGAG GCCGUUAGGC CGAA IAGGACAA | 8196 |
| 357 | UGUCCUCC A AUUUGUCC | 797 | GGACAAAU CUGAUGAG GCCGUUAGGC CGAA IGAGGACA | 8197 |
| 365 | AAUUUGUC C UGGUUAUC | 798 | GAUAACCA CUGAUGAG GCCGUUAGGC CGAA IACAAAUU | 8198 |
| 366 | AUUUGUCC U GGUUAUCG | 799 | CGAUAACC CUGAUGAG GCCGUUAGGC CGAA IGACAAAU | 8199 |
| 376 | GUUAUCGC U GGAUGUGU | 800 | ACACAUCC CUGAUGAG GCCGUUAGGC CGAA ICGAUAAC | 8200 |
| 386 | GAUGUGUC U GCGGCGUU | 801 | AACGCCGC CUGAUGAG GCCGUUAGGC CGAA IACACAUC | 8201 |
| 400 | GUUUUAUC A UCUUCCUC | 802 | GAGGAAGA CUGAUGAG GCCGUUAGGC CGAA IAUAAAAC | 8202 |
| 403 | UUAUCAUC U UCCUCUGC | 803 | GCAGAGGA CUGAUGAG GCCGUUAGGC CGAA IAUGAUAA | 8203 |
| 406 | UCAUCUUC C UCUGCAUC | 804 | GAUGCAGA CUGAUGAG GCCGUUAGGC CGAA IAAGAUGA | 8204 |
| 407 | CAUCUUCC U CUGCAUCC | 805 | GGAUGCAG CUGAUGAG GCCGUUAGGC CGAA IGAAGAUG | 8205 |
| 409 | UCUUCCUC U GCAUCCUG | 806 | CAGGAUGC CUGAUGAG GCCGUUAGGC CGAA IAGGAAGA | 8206 |
| 412 | UCCUCUGC A UCCUGCUG | 807 | CAGCAGGA CUGAUGAG GCCGUUAGGC CGAA ICAGAGGA | 8207 |
| 415 | UCUGCAUC C UGCUGCUA | 808 | UAGCAGCA CUGAUGAG GCCGUUAGGC CGAA IAUGCAGA | 8208 |
| 416 | CUGCAUCC U GCUGCUAU | 809 | AUAGCAGC CUGAUGAG GCCGUUAGGC CGAA IGAUGCAG | 8209 |
| 419 | CAUCCUGC U GCUAUGCC | 810 | GGCAUAGC CUGAUGAG GCCGUUAGGC CGAA ICAGGAUG | 8210 |
| 422 | CCUGCUGC U AUGCCUCA | 811 | UGAGGCAU CUGAUGAG GCCGUUAGGC CGAA ICAGCAGG | 8211 |
| 427 | UGCUAUGC C UCAUCUUC | 812 | GAAGAUGA CUGAUGAG GCCGUUAGGC CGAA ICAUAGCA | 8212 |
| 428 | GCUAUGCC U CAUCUUCU | 813 | AGAAGAUG CUGAUGAG GCCGUUAGGC CGAA IGCAUAGC | 8213 |
| 430 | UAUGCCUC A UCUUCUUG | 814 | CAAGAAGA CUGAUGAG GCCGUUAGGC CGAA IAGGCAUA | 8214 |
| 433 | GCCUCAUC U UCUUGUUG | 815 | CAACAAGA CUGAUGAG GCCGUUAGGC CGAA IAUGAGGC | 8215 |
| 436 | UCAUCUUC U UGUUGGUU | 816 | AACCAACA CUGAUGAG GCCGUUAGGC CGAA IAAGAUGA | 8216 |
| 446 | GUUGGUUC U UCUGGACU | 817 | AGUCCAGA CUGAUGAG GCCGUUAGGC CGAA IAACCAAC | 8217 |
| 449 | GGUUCUUC U GGACUAUC | 818 | GAUAGUCC CUGAUGAG GCCGUUAGGC CGAA IAAGAACC | 8218 |
| 454 | UUCUGGAC U AUCAAGGU | 819 | ACCUUGAU CUGAUGAG GCCGUUAGGC CGAA IUCCAGAA | 8219 |
| 458 | GGACUAUC A AGGUAUGU | 820 | ACAUACCU CUGAUGAG GCCGUUAGGC CGAA IAUAGUCC | 8220 |
| 470 | UAUGUUGC C CGUUUGUC | 821 | GACAAACG CUGAUGAG GCCGUUAGGC CGAA ICAACAUA | 8221 |
| 471 | AUGUUGCC C GUUUGUCC | 822 | GGACAAAC CUGAUGAG GCCGUUAGGC CGAA IGCAACAU | 8222 |
| 479 | CGUUUGUC C UCUAAUUC | 823 | GAAUUAGA CUGAUGAG GCCGUUAGGC CGAA IACAAACG | 8223 |
| 480 | GUUUGUCC U CUAAUUCC | 824 | GGAAUUAG CUGAUGAG GCCGUUAGGC CGAA IGACAAAC | 8224 |
| 482 | UUGUCCUC U AAUUCCAG | 825 | CUGGAAUU CUGAUGAG GCCGUUAGGC CGAA IAGGACAA | 8225 |
| 488 | UCUAAUUC C AGGAUCAU | 826 | AUGAUCCU CUGAUGAG GCCGUUAGGC CGAA IAAUUAGA | 8226 |
| 489 | CUAAUUCC A GGAUCAUC | 827 | GAUGAUCC CUGAUGAG GCCGUUAGGC CGAA IGAAUUAG | 8227 |
| 495 | CCAGGAUC A UCAACAAC | 828 | GUUGUUGA CUGAUGAG GCCGUUAGGC CGAA IAUCCUGG | 8228 |
| 498 | GGAUCAUC A ACAACCAG | 829 | CUGGUUGU CUGAUGAG GCCGUUAGGC CGAA IAUGAUCC | 8229 |
| 501 | UCAUCAAC A ACCAGCAC | 830 | GUGCUGGU CUGAUGAG GCCGUUAGGC CGAA IUUGAUGA | 8230 |
| 504 | UCAACAAC C AGCACCGG | 831 | CCGGUGCU CUGAUGAG GCCGUUAGGC CGAA IUUGUUGA | 8231 |
| 505 | CAACAACC A GCACCGGA | 832 | UCCGGUGC CUGAUGAG GCCGUUAGGC CGAA IGUUGUUG | 8232 |
| 508 | CAACCAGC A CCGGACCA | 833 | UGGUCCGG CUGAUGAG GCCGUUAGGC CGAA ICUGGUUG | 8233 |
| 510 | ACCAGCAC C GGACCAUG | 834 | CAUGGUCC CUGAUGAG GCCGUUAGGC CGAA IUGCUGGU | 8234 |
| 515 | CACCGGAC C AUGCAAAA | 835 | UUUUGCAU CUGAUGAG GCCGUUAGGC CGAA IUCCGGUG | 8235 |
| 516 | ACCGGACC A UGCAAAAC | 836 | GUUUUGCA CUGAUGAG GCCGUUAGGC CGAA IGUCCGGU | 8236 |
| 520 | GACCAUGC A AAACCUGC | 837 | GCAGGUUU CUGAUGAG GCCGUUAGGC CGAA ICAUGGUC | 8237 |
| 525 | UGCAAAAC C UGCACAAC | 838 | GUUGUGCA CUGAUGAG GCCGUUAGGC CGAA IUUUUGCA | 8238 |
| 526 | GCAAAACC U GCACAACU | 839 | AGUUGUGC CUGAUGAG GCCGUUAGGC CGAA IGUUUUGC | 8239 |
| 529 | AAACCUGC A CAACUCCU | 840 | AGGAGUUG CUGAUGAG GCCGUUAGGC CGAA ICAGGUUU | 8240 |
| 531 | ACCUGCAC A ACUCCUGC | 841 | GCAGGAGU CUGAUGAG GCCGUUAGGC CGAA IUGCAGGU | 8241 |
| 534 | UGCACAAC U CCUGCUCA | 842 | UGAGCAGG CUGAUGAG GCCGUUAGGC CGAA IUUGUGCA | 8242 |
| 536 | CACAACUC C UGCUCAAG | 843 | CUUGAGCA CUGAUGAG GCCGUUAGGC CGAA IAGUUGUG | 8243 |
| 537 | ACAACUCC U GCUCAAGG | 844 | CCUUGAGC CUGAUGAG GCCGUUAGGC CGAA IGAGUUGU | 8244 |
| 540 | ACUCCUGC U CAAGGAAC | 845 | GUUCCUUG CUGAUGAG GCCGUUAGGC CGAA ICAGGAGU | 8245 |
| 542 | UCCUGCUC A AGGAACCU | 846 | AGGUUCCU CUGAUGAG GCCGUUAGGC CGAA IAGCAGGA | 8246 |
| 549 | CAAGGAAC C UCUAUGUU | 847 | AACAUAGA CUGAUGAG GCCGUUAGGC CGAA IUUCCUUG | 8247 |
| 550 | AAGGAACC U CUAUGUUU | 848 | AAACAUAG CUGAUGAG GCCGUUAGGC CGAA IGUUCCUU | 8248 |
| 552 | GGAACCUC U AUGUUUCC | 849 | GGAAACAU CUGAUGAG GCCGUUAGGC CGAA IAGGUUCC | 8249 |
| 560 | UAUGUUUC C CUCAUGUU | 850 | AACAUGAG CUGAUGAC GCCGUUAGGC CGAA IAAACAUA | 8250 |
| 561 | AUGUUUCC C UCAUGUUG | 851 | CAACAUGA CUGAUGAG GCCGUUAGGC CGAA IGAAACAU | 8251 |
| 562 | UGUUUCCC U CAUGUUGC | 852 | GCAACAUG CUGAUGAG GCCGUUAGGC CGAA IGGAAACA | 8252 |
| 564 | UUUCCCUC A UGUUGCUG | 853 | CAGCAACA CUGAUGAG GCCGUUAGGC CGAA IAGGGAAA | 8253 |
| 571 | CAUGUUGC U GUACAAAA | 854 | UUUUGUAC CUGAUGAG GCCGUUAGGC CGAA ICAACAUG | 8254 |
| 576 | UGCUGUAC A AAACCUAC | 855 | GUAGGUUU CUGAUGAG GCCGUUAGGC CGAA IUACAGCA | 8255 |
| 581 | UACAAAAC C UACGGACG | 856 | CGUCCGUA CUGAUGAG GCCGUUAGGC CGAA IUUUUGUA | 8256 |
| 582 | ACAAAACC U ACGGACGG | 857 | CCGUCCGU CUGAUGAG GCCGUUAGGC CGAA IGUUUUGU | 8257 |
| 595 | ACGGAAAC U GCACCUGU | 858 | ACAGGUGC CUGAUGAG GCCGUUAGGC CGAA IUUUCCGU | 8258 |
| 598 | GAAACUGC A CCUGUAUU | 859 | AAUACAGG CUGAUGAG GCCGUUAGGC CGAA ICAGUUUC | 8259 |
| 600 | AACUGCAC C UGUAUUCC | 860 | GGAAUACA CUGAUGAG GCCGUUAGGC CGAA IUGCAGUU | 8260 |
| 601 | ACUGCACC U GUAUUCCC | 861 | GGGAAUAC CUGAUGAG GCCGUUAGGC CGAA IGUGCAGU | 8261 |
| 608 | CUGUAUUC C CAUCCCAU | 862 | AUGGGAUG CUGAUGAG GCCGUUAGGC CGAA IAAUACAG | 8262 |
| 609 | UGUAUUCC C AUCCCAUC | 863 | GAUGGGAU CUGAUGAG GCCGUUAGGC CGAA IGAAUACA | 8263 |
| 610 | GUAUUCCC A UCCCAUCA | 864 | UGAUGGGA CUGAUGAG GCCGUUAGGC CGAA IGGAAUAC | 8264 |
| 613 | UUCCCAUC C CAUCAUCU | 865 | AGAUGAUG CUGAUGAG GCCGUUAGGC CGAA IAUGGGAA | 8265 |
| 614 | UCCCAUCC C AUCAUCUU | 866 | AAGAUGAU CUGAUGAG GCCGUUAGGC CGAA IGAUGGGA | 8266 |
| 615 | CCCAUCCC A UCAUCUUG | 867 | CAAGAUGA CUGAUGAG GCCGUUAGGC CGAA IGGAUGGG | 8267 |
| 618 | AUCCCAUC A UCUUGGGC | 868 | GCCCAAGA CUGAUGAG GCCGUUAGGC CGAA IAUGGGAU | 8268 |
| 621 | CCAUCAUC U UGGGCUUU | 869 | AAAGCCCA CUGAUGAG GCCGUUAGGC CGAA IAUGAUGG | 8269 |
| 627 | UCUUGGGC U UUCGCAAA | 870 | UUUGCGAA CUGAUGAG GCCGUUAGGC CGAA ICCCAAGA | 8270 |
| 633 | GCUUUCGC A AAAUACCU | 871 | AGGUAUUU CUGAUGAG GCCGUUAGGC CGAA ICGAAAGC | 8271 |
| 640 | CAAAAUAC C UAUGGGAG | 872 | CUCCCAUA CUGAUGAG GCCGUUAGGC CGAA IUAUUUUG | 8272 |
| 641 | AAAAUACC U AUGGGAGU | 873 | ACUCCCAU CUGAUGAG GCCGUUAGGC CGAA IGUAUUUU | 8273 |
| 654 | GAGUGGGC C UCAGUCCG | 874 | CGGACUGA CUGAUGAG GCCGUUAGGC CGAA ICCCACUC | 8274 |
| 655 | AGUGGGCC U CAGUCCGU | 875 | ACGGACUG CUGAUGAG GCCGUUAGGC CGAA IGCCCACU | 8275 |
| 657 | UGGGCCUC A GUCCGUUU | 876 | AAACGGAC CUGAUGAG GCCGUUAGGC CGAA IAGGCCCA | 8276 |
| 661 | CCUCAGUC C GUUUCUCU | 877 | AGAGAAAC CUGAUGAG GCCGUUAGGC CGAA IACUGAGG | 8277 |
| 667 | UCCGUUUC U CUUGGCUC | 878 | GAGCCAAG CUGAUGAG GCCGUUAGGC CGAA IAAACGGA | 8278 |
| 669 | CGUUUCUC U UGGCUCAG | 879 | CUGAGCCA CUGAUGAG GCCGUUAGGC CGAA IAGAAACG | 8279 |
| 674 | CUCUUGGC U CAGUUUAC | 880 | GUAAACUG CUGAUGAG GCCGUUAGGC CGAA ICCAAGAG | 8280 |
| 676 | CUUGGCUC A GUUUACUA | 881 | UAGUAAAC CUGAUGAG GCCGUUAGGC CGAA IAGCCAAG | 8281 |
| 683 | CAGUUUAC U AGUGCCAU | 882 | AUGGCACU CUGAUGAG GCCGUUAGGC CGAA IUAAACUG | 8282 |
| 689 | ACUAGUGC C AUUUGUUC | 883 | GAACAAAU CUGAUGAG GCCGUUAGGC CGAA ICACUAGU | 8283 |
| 690 | CUAGUGCC A UUUGUUCA | 884 | UGAACAAA CUGAUGAG GCCGUUAGGC CGAA IGCACUAG | 8284 |
| 698 | AUUUGUUC A GUGGUUCG | 885 | CGAACCAC CUGAUGAG GCCGUUAGGC CGAA IAACAAAU | 8285 |
| 713 | CGUAGGGC U UUCCCCCA | 886 | UGGGGGAA CUGAUGAG GCCGUUAGGC CGAA ICCCUACG | 8286 |
| 717 | GGGCUUUC C CCCACUGU | 887 | ACAGUGGG CUGAUGAG GCCGUUAGGC CGAA IAAAGCCC | 8287 |
| 718 | GGCUUUCC C CCACUGUC | 888 | GACAGUGG CUGAUGAG GCCGUUAGGC CGAA IGAAAGCC | 8288 |
| 719 | GCUUUCCC C CACUGUCU | 889 | AGACAGUG CUGAUGAG GCCGUUAGGC CGAA IGGAAAGC | 8289 |
| 720 | CUUUCCCC C ACUGUCUG | 890 | CAGACAGU CUGAUGAG GCCGUUAGGC CGAA IGGGAAAG | 8290 |
| 721 | UUUCCCCC A CUGUCUGG | 891 | CCAGACAG CUGAUGAG GCCGUUAGGC CGAA IGGGGAAA | 8291 |
| 723 | UCCCCCAC U GUCUGGCU | 892 | AGCCAGAC CUGAUGAG GCCGUUAGGC CGAA IUGGGGGA | 8292 |
| 727 | CCACUGUC U GGCUUUCA | 893 | UGAAAGCC CUGAUGAG GCCGUUAGGC CGAA IACAGUGG | 8293 |
| 731 | UGUCUGGC U UUCAGUUA | 894 | UAACUGAA CUGAUGAG GCCGUUAGGC CGAA ICCAGACA | 8294 |
| 735 | UGGCUUUC A GUUAUAUG | 895 | CAUAUAAC CUGAUGAG GCCGUUAGGC CGAA IAAAGCCA | 8295 |
| 764 | UUGGGGGC C AAGUCUGU | 896 | ACAGACUU CUGAUGAG GCCGUUAGGC CGAA ICCCCCAA | 8296 |
| 765 | UGGGGGCC A AGUCUGUA | 897 | UACAGACU CUGAUGAG GCCGUUAGGC CGAA IGCCCCCA | 8297 |
| 770 | GCCAAGUC U GUACAACA | 898 | UGUUGUAC CUGAUGAG GCCGUUAGGC CGAA IACUUGGC | 8298 |
| 775 | GUCUGUAC A ACAUCUUG | 899 | CAAGAUGU CUGAUGAG GCCGUUAGGC CGAA IUACAGAC | 8299 |
| 778 | UGUACAAC A UCUUGAGU | 900 | ACUCAAGA CUGAUGAG GCCGUUAGGC CGAA IUUGUACA | 8300 |
| 781 | ACAACAUC U UGAGUCCC | 901 | GGGACUCA CUGAUGAG GCCGUUAGGC CGAA IAUGUUGU | 8301 |
| 788 | CUUGAGUC C CUUUAUGC | 902 | GCAUAAAG CUGAUGAG GCCGUUAGGC CGAA IACUCAAG | 8302 |
| 789 | UUGAGUCC C UUUAUGCC | 903 | GGCAUAAA CUGAUGAG GCCGUUAGGC CGAA IGACUCAA | 8303 |
| 790 | UGAGUCCC U UUAUGCCG | 904 | CGGCAUAA CUGAUGAG GCCGUUAGGC CGAA IGGACUCA | 8304 |
| 797 | CUUUAUGC C GCUGUUAC | 905 | GUAACAGC CUGAUGAG GCCGUUAGGC CGAA ICAUAAAG | 8305 |
| 800 | UAUGCCGC U GUUACCAA | 906 | UUGGUAAC CUGAUGAG GCCGUUAGGC CGAA ICGGCAUA | 8306 |
| 806 | GCUGUUAC C AAUUUUCU | 907 | AGAAAAUU CUGAUGAG GCCGUUAGGC CGAA IUAACAGC | 8307 |
| 807 | CUGUUACC A AUUUUCUU | 908 | AAGAAAAU CUGAUGAG GCCGUUAGGC CGAA IGUAACAG | 8308 |
| 814 | CAAUUUUC U UUUGUCUU | 909 | AAGACAAA CUGAUGAG GCCGUUAGGC CGAA IAAAAUUG | 8309 |
| 821 | CUUUUGUC U UUGGGUAU | 910 | AUACCCAA CUGAUGAG GCCGUUAGGC CGAA IACAAAAG | 8310 |
| 832 | GGGUAUAC A UUUAAACC | 911 | GGUUUAAA CUGAUGAG GCCGUUAGGC CGAA IUAUACCC | 8311 |
| 840 | AUUUAAAC C CUCACAAA | 912 | UUUGUGAG CUGAUGAG GCCGUUAGGC CGAA IUUUAAAU | 8312 |
| 841 | UUUAAACC C UCACAAAA | 913 | UUUUGUGA CUGAUGAG GCCGUUAGGC CGAA IGUUUAAA | 8313 |
| 842 | UUAAACCC U CACAAAAC | 914 | GUUUUGUG CUGAUGAG GCCGUUAGGC CGAA IGGUUUAA | 8314 |
| 844 | AAACCCUC A CAAAACAA | 915 | UUGUUUUG CUGAUGAG GCCGUUAGGC CGAA IAGGGUUU | 8315 |
| 846 | ACCCUCAC A AAACAAAA | 916 | UUUUGUUU CUGAUGAG GCCGUUAGGC CGAA IUGAGGGU | 8316 |
| 851 | CACAAAAC A AAAAGAUG | 917 | CAUCUUUU CUGAUGAG GCCGUUAGGC CGAA IUUUUGUG | 8317 |
| 869 | GGAUAUUC C CUUAACUU | 918 | AAGUUAAG CUGAUGAG GCCGUUAGGC CGAA IAAUAUCC | 8318 |
| 870 | GAUAUUCC C UUAACUUC | 919 | GAAGUUAA CUGAUGAG GCCGUUAGGC CGAA IGAAUAUC | 8319 |
| 871 | AUAUUCCC U UAACUUCA | 920 | UGAAGUUA CUGAUGAG GCCGUUAGGC CGAA IGGAAUAU | 8320 |
| 876 | CCCUUAAC U UCAUGGGA | 921 | UCCCAUGA CUGAUGAG GCCGUUAGGC CGAA IUUAAGGG | 8321 |
| 879 | UUAACUUC A UGGGAUAU | 922 | AUAUCCCA CUGAUGAG GCCGUUAGGC CGAA IAAGUUAA | 8322 |
| 906 | GUUGGGGC A CAUUGCCA | 923 | UGGCAAUG CUGAUGAG GCCGUUAGGC CGAA ICCCCAAC | 8323 |
| 908 | UGGGGCAC A UUGCCACA | 924 | UGUGGCAA CUGAUGAG GCCGUUAGGC CGAA IUGCCCCA | 8324 |
| 913 | CACAUUGC C ACAGGAAC | 925 | GUUCCUGU CUGAUGAG GCCGUUAGGC CGAA ICAAUGUG | 8325 |
| 914 | ACAUUGCC A CAGGAACA | 926 | UGUUCCUG CUGAUGAG GCCGUUAGGC CGAA IGCAAUGU | 8326 |
| 916 | AUUGCCAC A GGAACAUA | 927 | UAUGUUCC CUGAUGAG GCCGUUAGGC CGAA IUGGCAAU | 8327 |
| 922 | ACAGGAAC A UAUUGUAC | 928 | GUACAAUA CUGAUGAG GCCGUUAGGC CGAA IUUCCUGU | 8328 |
| 931 | UAUUGUAC A AAAAAUCA | 929 | UGAUUUUU CUGAUGAG GCCGUUAGGC CGAA IUACAAUA | 8329 |
| 939 | AAAAAAUC A AAAUGUGU | 930 | ACACAUUU CUGAUGAG GCCGUUAGGC CGAA IAUUUUUU | 8330 |
| 958 | UAGGAAAC U UCCUGUAA | 931 | UUACAGGA CUGAUGAG GCCGUUAGGC CGAA IUUUCCUA | 8331 |
| 961 | GAAACUUC C UGUAAACA | 932 | UGUUUACA CUGAUGAG GCCGUUAGGC CGAA IAAGUUUC | 8332 |
| 962 | AAACUUCC U GUAAACAG | 933 | CUGUUUAC CUGAUGAG GCCGUUAGGC CGAA IGAAGUUU | 8333 |
| 969 | CUGUAAAC A GGCCUAUU | 934 | AAUAGGCC CUGAUGAG GCCGUUAGGC CGAA IUUUACAG | 8334 |
| 973 | AAACAGGC C UAUUGAUU | 935 | AAUCAAUA CUGAUGAG GCCGUUAGGC CGAA ICCUGUUU | 8335 |
| 974 | AACAGGCC U AUUGAUUG | 936 | CAAUCAAU CUGAUGAG GCCGUUAGGC CGAA IGCCUGUU | 8336 |
| 994 | AGUAUGUC A ACGAAUUG | 937 | CAAUUCGU CUGAUGAG GCCGUUAGGC CGAA IACAUACU | 8337 |
| 1009 | UGUGGGUC U UUUGGGGU | 938 | ACCCCAAA CUGAUGAG GCCGUUAGGC CGAA IACCCACA | 8338 |
| 1022 | GGGUUUGC C GCCCCUUU | 939 | AAAGGGGC CUGAUGAG GCCGUUAGGC CGAA ICAAACCC | 8339 |
| 1025 | UUUGCCGC C CCUUUCAC | 940 | GUGAAAGG CUGAUGAG GCCGUUAGGC CGAA ICGGCAAA | 8340 |
| 1026 | UUGCCGCC C CUUUCACG | 941 | CGUGAAAG CUGAUGAG GCCGUUAGGC CGAA IGCGGCAA | 8341 |
| 1027 | UGCCGCCC C UUUCACGC | 942 | GCGUGAAA CUGAUGAG GCCGUUAGGC CGAA IGGCGGCA | 8342 |
| 1028 | GCCGCCCC U UUCACGCA | 943 | UGCGUGAA CUGAUGAG GCCGUUAGGC CGAA IGGGCGGC | 8343 |
| 1032 | CCCCUUUC A CGCAAUGU | 944 | ACAUUGCG CUGAUGAG GCCGUUAGGC CGAA IAAAGGGG | 8344 |
| 1036 | UUUCACGC A AUGUGGAU | 945 | AUCCACAU CUGAUGAG GCCGUUAGGC CGAA ICGUGAAA | 8345 |
| 1049 | GGAUAUUC U GCUUUAAU | 946 | AUUAAAGC CUGAUGAG GCCGUUAGGC CGAA IAAUAUCC | 8346 |
| 1052 | UAUUCUGC U UUAAUGCC | 947 | GGCAUUAA CUGAUGAG GCCGUUAGGC CGAA ICAGAAUA | 8347 |
| 1060 | UUUAAUGC C UUUAUAUG | 948 | CAUAUAAA CUGAUGAG GCCGUUAGGC CGAA ICAUUAAA | 8348 |
| 1061 | UUAAUGCC U UUAUAUGC | 949 | GCAUAUAA CUGAUGAG GCCGUUAGGC CGAA IGCAUUAA | 8349 |
| 1070 | UUAUAUGC A UGCAUACA | 950 | UGUAUGCA CUGAUGAG GCCGUUAGGC CGAA ICAUAUAA | 8350 |
| 1074 | AUGCAUGC A UACAAGCA | 951 | UGCUUGUA CUGAUGAG GCCGUUAGGC CGAA ICAUGCAU | 8351 |
| 1078 | AUGCAUAC A AGCAAAAC | 952 | GUUUUGCU CUGAUCAG GCCGUUAGGC CGAA IUAUGCAU | 8352 |
| 1082 | AUACAAGC A AAACAGGC | 953 | GCCUGUUU CUGAUGAG GCCGUUAGGC CGAA ICUUGUAU | 8353 |
| 1087 | AGCAAAAC A GGCUUUUA | 954 | UAAAAGCC CUGAUGAG GCCGUUAGGC CGAA IUUUUGCU | 8354 |
| 1091 | AAACAGGC U UUUACUUU | 955 | AAAGUAAA CUGAUGAG GCCGUUAGGC CGAA ICCUGUUU | 8355 |
| 1097 | GCUUUUAC U UUCUCGCC | 956 | GGCGAGAA CUGAUGAG GCCGUUAGGC CGAA IUAAAAGC | 8356 |
| 1101 | UUACUUUC U CGCCAACU | 957 | AGUUGGCG CUGAUGAG GCCGUUAGGC CGAA IAAAGUAA | 8357 |
| 1105 | UUUCUCGC C AACUUACA | 958 | UGUAAGUU CUGAUGAG GCCGUUAGGC CGAA ICGAGAAA | 8358 |
| 1106 | UUCUCGCC A ACUUACAA | 959 | UUGUAAGU CUGAUGAG GCCGUUAGGC CGAA IGCGAGAA | 8359 |
| 1109 | UCGCCAAC U UACAAGGC | 960 | GCCUUGUA CUGAUGAG GCCGUUAGGC CGAA IUUGGCGA | 8360 |
| 1113 | CAACUUAC A AGGCCUUU | 961 | AAAGGCCU CUGAUGAG GCCGUUAGGC CGAA IUAAGUUG | 8361 |
| 1118 | UACAAGGC C UUUCUAAG | 962 | CUUAGAAA CUGAUGAG GCCGUUAGGC CGAA ICCUUGUA | 8362 |
| 1119 | ACAAGGCC U UUCUAAGU | 963 | ACUUAGAA CUGAUGAG GCCGUUAGGC CGAA IGCCUUGU | 8363 |
| 1123 | GGCCUUUC U AAGUAAAC | 964 | GUUUACUU CUGAUGAG GCCGUUAGGC CGAA IAAAGGCC | 8364 |
| 1132 | AAGUAAAC A GUAUGUGA | 965 | UCACAUAC CUGAUGAG GCCGUUAGGC CGAA IUUUACUU | 8365 |
| 1143 | AUGUGAAC C UUUACCCC | 966 | GGGGUAAA CUGAUGAG GCCGUUAGGC CGAA IUUCACAU | 8366 |
| 1144 | UGUGAACC U UUACCCCG | 967 | CGGGGUAA CUGAUGAG GCCGUUAGGC CGAA IGUUCACA | 8367 |
| 1149 | ACCUUUAC C CCGUUGCU | 968 | AGCAACGG CUGAUGAG GCCGUUAGGC CGAA IUAAAGGU | 8368 |
| 1150 | CCUUUACC C CGUUGCUC | 969 | GAGCAACG CUGAUGAG GCCGUUAGGC CGAA IGUAAAGG | 8369 |
| 1151 | CUUUACCC C GUUGCUCG | 970 | CGAGCAAC CUGAUGAG GCCGUUAGGC CGAA IGGUAAAG | 8370 |
| 1157 | CCCGUUGC U CGGCAACG | 971 | CGUUGCCG CUGAUGAG GCCGUUAGGC CGAA ICAACGGG | 8371 |
| 1162 | UGCUCGGC A ACGGCCUG | 972 | CAGGCCGU CUGAUGAG GCCGUUAGGC CGAA ICCGAGCA | 8372 |
| 1168 | GCAACGGC C UGGUCUAU | 973 | AUAGACCA CUGAUGAG GCCGUUAGGC CGAA ICCGUUGC | 8373 |
| 1169 | CAACGGCC U GGUCUAUG | 974 | CAUAGACC CUGAUGAG GCCGUUAGGC CGAA IGCCGUUG | 8374 |
| 1174 | GCCUGGUC U AUGCCAAG | 975 | CUUGGCAU CUGAUGAG GCCGUUAGGC CGAA IACCAGGC | 8375 |
| 1179 | GUCUAUGC C AAGUGUUU | 976 | AAACACUU CUGAUGAG GCCGUUAGGC CGAA ICAUAGAC | 8376 |
| 1180 | UCUAUGCC A AGUGUUUG | 977 | CAAACACU CUGAUGAG GCCGUUAGGC CGAA IGCAUAGA | 8377 |
| 1190 | GUGUUUGC U GACGCAAC | 978 | GUUGCGUC CUGAUGAG GCCGUUAGGC CGAA ICAAACAC | 8378 |
| 1196 | GCUGACGC A ACCCCCAC | 979 | GUGGGGGU CUGAUGAG GCCGUUAGGC CGAA ICGUCAGC | 8379 |
| 1199 | GACGCAAC C CCCACUGG | 980 | CCAGUGGG CUGAUGAG GCCGUUAGGC CGAA IUUGCGUC | 8380 |
| 1200 | ACGCAACC C CCACUGGU | 981 | ACCAGUGG CUGAUGAG GCCGUUAGGC CGAA IGUUGCGU | 8381 |
| 1201 | CGCAACCC C CACUGGUU | 982 | AACCAGUG CUGAUGAG GCCGUUAGGC CGAA IGGUUGCG | 8382 |
| 1202 | GCAACCCC C ACUGGUUG | 983 | CAACCAGU CUGAUGAG GCCGUUAGGC CGAA IGGGUUGC | 8383 |
| 1203 | CAACCCCC A CUGGUUGG | 984 | CCAACCAG CUGAUGAG GCCGUUAGGC CGAA IGGGGUUG | 8384 |
| 1205 | ACCCCCAC U GGUUGGGG | 985 | CCCCAACC CUGAUGAG GCCGUUAGGC CGAA IUGGGGGU | 8385 |
| 1215 | GUUGGGGC U UGGCCAUA | 986 | UAUGGCCA CUGAUGAG GCCGUUAGGC CGAA ICCCCAAC | 8386 |
| 1220 | GGCUUGGC C AUAGGCCA | 987 | UGGCCUAU CUGAUGAG GCCGUUAGGC CGAA ICCAAGCC | 8387 |
| 1221 | GCUUGGCC A UAGGCCAU | 988 | AUGGCCUA CUGAUGAG GCCGUUAGGC CGAA IGCCAAGC | 8388 |
| 1227 | CCAUAGGC C AUCAGCGC | 989 | GCGCUGAU CUGAUGAG GCCGUUAGGC CGAA ICCUAUGG | 8389 |
| 1228 | CAUAGGCC A UCAGCGCA | 990 | UGCGCUGA CUGAUGAG GCCGUUAGGC CGAA IGCCUAUG | 8390 |
| 1231 | AGGCCAUC A GCGCAUGC | 991 | GCAUGCGC CUGAUGAG GCCGUUAGGC CGAA IAUGGCCU | 8391 |
| 1236 | AUCAGCGC A UGCGUGGA | 992 | UCCACGCA CUGAUGAG GCCGUUAGGC CGAA ICGCUGAU | 8392 |
| 1247 | CGUGGAAC C UUUGUGUC | 993 | GACACAAA CUGAUGAG GCCGUUAGGC CGAA IUUCCACG | 8393 |
| 1248 | GUGGAACC U UUGUGUCU | 994 | AGACACAA CUGAUGAG GCCGUUAGGC CGAA IGUUCCAC | 8394 |
| 1256 | UUUGUGUC U CCUCUGCC | 995 | GGCAGAGG CUGAUGAG GCCGUUAGGC CGAA IACACAAA | 8395 |
| 1258 | UGUGUCUC C UCUGCCGA | 996 | UCGGCAGA CUGAUGAG GCCGUUAGGC CGAA IAGACACA | 8396 |
| 1259 | GUGUCUCC U CUGCCGAU | 997 | AUCGGCAG CUGAUGAG GCCGUUAGGC CGAA IGAGACAC | 8397 |
| 1261 | GUCUCCUC U GCCGAUCC | 998 | GGAUCGGC CUGAUGAG GCCGUUAGGC CGAA IAGGAGAC | 8398 |
| 1264 | UCCUCUGC C GAUCCAUA | 999 | UAUGGAUC CUGAUGAG GCCGUUAGGC CGAA ICAGAGGA | 8399 |
| 1269 | UGCCGAUC C AUACCGCG | 1000 | CGCGGUAU CUGAUGAG GCCGUUAGGC CGAA IAUCGGCA | 8400 |
| 1270 | GCCGAUCC A UACCGCGG | 1001 | CCGCGGUA CUGAUGAG GCCGUUAGGC CGAA IGAUCGGC | 8401 |
| 1274 | AUCCAUAC C GCGGAACU | 1002 | AGUUCCGC CUGAUGAG GCCGUUAGGC CGAA IUAUGGAU | 8402 |
| 1282 | CGCGGAAC U CCUAGCCG | 1003 | CGGCUAGG CUGAUGAG GCCGUUAGGC CGAA IUUCCGCG | 8403 |
| 1284 | CGGAACUC C UAGCCGCU | 1004 | AGCGGCUA CUGAUGAG GCCGUUAGGC CGAA IAGUUCCG | 8404 |
| 1285 | GGAACUCC U AGCCGCUU | 1005 | AAGCGGCU CUGAUGAG GCCGUUAGGC CGAA IGAGUUCC | 8405 |
| 1289 | CUCCUAGC C GCUUGUUU | 1006 | AAACAAGC CUGAUGAG GCCGUUAGGC CGAA ICUAGGAG | 8406 |
| 1292 | CUAGCCGC U UGUUUUGC | 1007 | GCAAAACA CUGAUGAG GCCGUUAGGC CGAA ICGGCUAG | 8407 |
| 1301 | UGUUUUGC U CGCAGCAG | 1008 | CUGCUGCG CUGAUGAG GCCGUUAGGC CGAA ICAAAACA | 8408 |
| 1305 | UUGCUCGC A GCAGGUCU | 1009 | AGACCUGC CUGAUGAG GCCGUUAGGC CGAA ICGAGCAA | 8409 |
| 1308 | CUCGCAGC A GGUCUGGG | 1010 | CCCAGACC CUGAUGAG GCCGUUAGGC CGAA ICUGCGAG | 8410 |
| 1313 | AGCAGGUC U GGGGCAAA | 1011 | UUUGCCCC CUGAUGAG GCCGUUAGGC CGAA IACCUGCU | 8411 |
| 1319 | UCUGGGGC A AAACUCAU | 1012 | AUGAGUUU CUGAUGAG GCCGUUAGGC CGAA ICCCCAGA | 8412 |
| 1324 | GGCAAAAC U CAUCGGGA | 1013 | UCCCGAUG CUGAUGAG GCCGUUAGGC CGAA IUUUUGCC | 8413 |
| 1326 | CAAAACUC A UCGGGACU | 1014 | AGUCCCGA CUGAUGAG GCCGUUAGGC CGAA IAGUUUUG | 8414 |
| 1334 | AUCGGGAC U GACAAUUC | 1015 | GAAUUGUC CUGAUGAG GCCGUUAGGC CGAA IUCCCGAU | 8415 |
| 1338 | GGACUGAC A AUUCUGUC | 1016 | GACAGAAU CUGAUGAG GCCGUUAGGC CGAA IUCAGUCC | 8416 |
| 1343 | GACAAUUC U GUCGUGCU | 1017 | AGCACGAC CUGAUGAG GCCGUUAGGC CGAA IAAUUGUC | 8417 |
| 1351 | UGUCGUGC U CUCCCGCA | 1018 | UGCGGGAG CUGAUGAG GCCGUUAGGC CGAA ICACGACA | 8418 |
| 1353 | UCGUGCUC U CCCGCAAA | 1019 | UUUGCGGG CUGAUGAG GCCGUUAGGC CGAA IAGCACGA | 8419 |
| 1355 | GUGCUCUC C CGCAAAUA | 1020 | UAUUUGCG CUGAUGAG GCCGUUAGGC CGAA IAGAGCAC | 8420 |
| 1356 | UGCUCUCC C GCAAAUAU | 1021 | AUAUUUGC CUGAUGAG GCCGUUAGGC CGAA IGAGAGCA | 8421 |
| 1359 | UCUCCCGC A AAUAUACA | 1022 | UGUAUAUU CUGAUGAG GCCGUUAGGC CGAA ICGGGAGA | 8422 |
| 1367 | AAAUAUAC A UCAUUUCC | 1023 | GGAAAUGA CUGAUGAG GCCGUUAGGC CGAA IUAUAUUU | 8423 |
| 1370 | UAUACAUC A UUUCCAUG | 1024 | CAUGGAAA CUGAUGAG GCCGUUAGGC CGAA IAUGUAUA | 8424 |
| 1375 | AUCAUUUC C AUGGCUGC | 1025 | GCAGCCAU CUGAUGAG GCCGUUAGGC CGAA IAAAUGAU | 8425 |
| 1376 | UCAUUUCC A UGGCUGCU | 1026 | AGCAGCCA CUGAUGAG GCCGUUAGGC CGAA IGAAAUGA | 8426 |
| 1381 | UCCAUGGC U GCUAGGCU | 1027 | AGCCUAGC CUGAUGAG GCCGUUAGGC CGAA ICCAUGGA | 8427 |
| 1384 | AUGGCUGC U AGGCUGUG | 1028 | CACAGCCU CUGAUGAG GCCGUUAGGC CGAA ICAGCCAU | 8428 |
| 1389 | UGCUAGGC U GUGCUGCC | 1029 | GGCAGCAC CUGAUGAG GCCGUUAGGC CGAA ICCUAGCA | 8429 |
| 1394 | GGCUGUGC U GCCAACUG | 1030 | CAGUUGGC CUGAUGAG GCCGUUAGGC CGAA ICACAGCC | 8430 |
| 1397 | UGUGCUGC C AACUGGAU | 1031 | AUCCAGUU CUGAUGAG GCCGUUAGGC CGAA ICAGCACA | 8431 |
| 1398 | GUGCUGCC A ACUGGAUC | 1032 | GAUCCAGU CUGAUGAG GCCGUUAGGC CGAA IGCAGCAC | 8432 |
| 1401 | CUGCCAAC U GGAUCCUA | 1033 | UAGGAUCC CUGAUGAG GCCGUUAGGC CGAA IUUGGCAG | 8433 |
| 1407 | ACUGGAUC C UACGCGGG | 1034 | CCCGCGUA CUGAUGAG GCCGUUAGGC CGAA IAUCCAGU | 8434 |
| 1408 | CUGGAUCC U ACGCGGGA | 1035 | UCCCGCGU CUGAUGAG GCCGUUAGGC CGAA IGAUCCAG | 8435 |
| 1421 | GGGACGUC C UUUGUUUA | 1036 | UAAACAAA CUGAUGAG GCCGUUAGGC CGAA IACGUCCC | 8436 |
| 1422 | GGACGUCC U UUGUUUAC | 1037 | GUAAACAA CUGAUGAG GCCGUUAGGC CGAA IGACGUCC | 8437 |
| 1434 | UUUACGUC C CGUCGGCG | 1038 | CGCCGACG CUGAUGAG GCCGUUAGGC CGAA IACGUAAA | 8438 |
| 1435 | UUACGUCC C GUCGGCGC | 1039 | GCGCCGAC CUGAUGAG GCCGUUAGGC CGAA IGACGUAA | 8439 |
| 1444 | GUCGGCGC U GAAUCCCG | 1040 | CGGGAUUC CUGAUGAG GCCGUUAGGC CGAA ICGCCGAC | 8440 |
| 1450 | GCUGAAUC C CGCGGACG | 1041 | CGUCCGCG CUGAUGAG GCCGUUAGGC CGAA IAUUCAGC | 8441 |
| 1451 | CUGAAUCC C GCGGACGA | 1042 | UCGUCCGC CUGAUGAG GCCGUUAGGC CGAA IGAUUCAG | 8442 |
| 1461 | CGGACGAC C CCUCCCGG | 1043 | CCGGGAGG CUGAUGAG GCCGUUAGGC CGAA IUCGUCCG | 8443 |
| 1462 | GGACGACC C CUCCCGGG | 1044 | CCCGGGAG CUGAUGAG GCCGUUAGGC CGAA IGUCGUCC | 8444 |
| 1463 | GACGACCC C UCCCGGGG | 1045 | CCCCGGGA CUGAUGAG GCCGUUAGGC CGAA IGGUCGUC | 8445 |
| 1464 | ACGACCCC U CCCGGGGC | 1046 | GCCCCGGG CUGAUGAG GCCGUUAGGC CGAA IGGGUCGU | 8446 |
| 1466 | GACCCCUC C CGGGGCCG | 1047 | CGGCCCCG CUGAUGAG GCCGUUAGGC CGAA IAGGGGUC | 8447 |
| 1467 | ACCCCUCC C GGGGCCGC | 1048 | GCGGCCCC CUGAUGAG GCCGUUAGGC CGAA IGAGGGGU | 8448 |
| 1473 | CCCGGGGC C GCUUGGGG | 1049 | CCCCAAGC CUGAUGAG GCCGUUAGGC CGAA ICCCCGGG | 8449 |
| 1476 | GGGGCCGC U UGGGGCUC | 1050 | GAGCCCCA CUGAUGAG GCCGUUAGGC CGAA ICGGCCCC | 8450 |
| 1483 | CUUGGGGC U CUACCGCC | 1051 | GGCGGUAG CUGAUGAG GCCGUUAGGC CGAA ICCCCAAG | 8451 |
| 1485 | UGGGGCUC U ACCGCCCG | 1052 | CGGGCGGU CUGAUGAG GCCGUUAGGC CGAA IAGCCCCA | 8452 |
| 1488 | GGCUCUAC C GCCCGCUU | 1053 | AAGCGGGC CUGAUGAG GCCGUUAGGC CGAA IUAGAGCC | 8453 |
| 1491 | UCUACCGC C CGCUUCUC | 1054 | GAGAAGCG CUGAUGAG GCCGUUAGGC CGAA ICGGUAGA | 8454 |
| 1492 | CUACCGCC C GCUUCUCC | 1055 | GGAGAAGC CUGAUGAG GCCGUUAGGC CGAA IGCGGUAG | 8455 |
| 1495 | CCGCCCGC U UCUCCGCC | 1056 | GGCGGAGA CUGAUGAG GCCGUUAGGC CGAA ICGGGCGG | 8456 |
| 1498 | CCCGCUUC U CCGCCUAU | 1057 | AUAGGCGG CUGAUGAG GCCGUUAGGC CGAA IAAGCGGG | 8457 |
| 1500 | CGCUUCUC C GCCUAUUG | 1058 | CAAUAGGC CUGAUGAG GCCGUUAGGC CGAA IAGAAGCG | 8458 |
| 1503 | UUCUCCGC C UAUUGUAC | 1059 | GUACAAUA CUGAUGAG GCCGUUAGGC CGAA ICGGAGAA | 8459 |
| 1504 | UCUCCGCC U AUUGUACC | 1060 | GGUACAAU CUGAUGAG GCCGUUAGGC CGAA IGCGGAGA | 8460 |
| 1512 | UAUUGUAC C GACCGUCC | 1061 | GGACGGUC CUGAUGAG GCCGUUAGGC CGAA IUACAAUA | 8461 |
| 1516 | GUACCGAC C GUCCACGG | 1062 | CCGUGGAC CUGAUGAG GCCGUUAGGC CGAA IUCGGUAC | 8462 |
| 1520 | CGACCGUC C ACGGGGCG | 1063 | CGCCCCGU CUGAUGAG GCCGUUAGGC CGAA IACGGUCG | 8463 |
| 1521 | GACCGUCC A CGGGGCGC | 1064 | GCGCCCCG CUGAUGAG GCCGUUAGGC CGAA IGACGGUC | 8464 |
| 1530 | CGGGGCGC A CCUCUCUU | 1065 | AAGAGAGG CUGAUGAG GCCGUUAGGC CGAA ICGCCCCG | 8465 |
| 1532 | GGGCGCAC C UCUCUUUA | 1066 | UAAAGAGA CUGAUGAG GCCGUUAGGC CGAA IUGCGCCC | 8466 |
| 1533 | GGCGCACC U CUCUUUAC | 1067 | GUAAAGAG CUGAUGAG GCCGUUAGGC CGAA IGUGCGCC | 8467 |
| 1535 | CGCACCUC U CUUUACGC | 1068 | GCGUAAAG CUGAUGAG GCCGUUAGGC CGAA IAGGUGCG | 8468 |
| 1537 | CACCUCUC U UUACGCGG | 1069 | CCGCGUAA CUGAUGAG GCCGUUAGGC CGAA IAGAGGUG | 8469 |
| 1548 | ACGCGGAC U CCCCGUCU | 1070 | AGACGGGG CUGAUGAG GCCGUUAGGC CGAA IUCCGCGU | 8470 |
| 1550 | GCGGACUC C CCGUCUGU | 1071 | ACAGACGG CUGAUGAG GCCGUUAGGC CGAA IAGUCCGC | 8471 |
| 1551 | CGGACUCC C CGUCUGUG | 1072 | CACAGACG CUGAUGAG GCCGUUAGGC CGAA IGAGUCCG | 8472 |
| 1552 | GGACUCCC C GUCUGUGC | 1073 | GCACAGAC CUGAUGAG GCCGUUAGGC CGAA IGGAGUCC | 8473 |
| 1556 | UCCCCGUC U GUGCCUUC | 1074 | GAAGGCAC CUGAUGAG GCCGUUAGGC CGAA IACGGGGA | 8474 |
| 1561 | GUCUGUGC C UUCUCAUC | 1075 | GAUGAGAA CUGAUGAG GCCGUUAGGC CGAA ICACAGAC | 8475 |
| 1562 | UCUGUGCC U UCUCAUCU | 1076 | AGAUGAGA CUGAUGAG GCCGUUAGGC CGAA IGCACAGA | 8476 |
| 1565 | GUGCCUUC U CAUCUGCC | 1077 | GGCAGAUG CUGAUGAG GCCGUUAGGC CGAA IAAGGCAC | 8477 |
| 1567 | GCCUUCUC A UCUGCCGG | 1078 | CCGGCAGA CUGAUGAG GCCGUUAGGC CGAA IAGAAGGC | 8478 |
| 1570 | UUCUCAUC U GCCGGACC | 1079 | GGUCCGGC CUGAUGAG GCCGUUAGGC CGAA IAUGAGAA | 8479 |
| 1573 | UCAUCUGC C GGACCGUG | 1080 | CACGGUCC CUGAUGAG GCCGUUAGGC CGAA ICAGAUGA | 8480 |
| 1578 | UGCCGGAC C GUGUCCAC | 1081 | GUGCACAC CUGAUGAG GCCGUUAGGC CGAA IUCCGGCA | 8481 |
| 1585 | CCGUGUGC A CUUCGCUU | 1082 | AAGCGAAG CUGAUGAG GCCGUUAGGC CGAA ICACACGG | 8482 |
| 1587 | GUGUGCAC U UCGCUUCA | 1083 | UGAAGCGA CUGAUGAG GCCGUUAGGC CGAA IUGCACAC | 8483 |
| 1592 | CACUUCGC U UCACCUCU | 1084 | AGAGGUGA CUGAUGAG GCCGUUAGGC CGAA ICGAAGUG | 8484 |
| 1595 | UUCGCUUC A CCUCUGCA | 1085 | UGCAGAGG CUGAUGAG GCCGUUAGGC CGAA IAAGCGAA | 8485 |
| 1597 | CGCUUCAC C UCUGCACG | 1086 | CGUGCAGA CUGAUGAG GCCGUUAGGC CGAA IUGAAGCG | 8486 |
| 1598 | GCUUCACC U CUGCACGU | 1087 | ACGUGCAG CUGAUGAG GCCGUUAGGC CGAA IGUGAAGC | 8487 |
| 1600 | UUCACCUC U GCACGUCG | 1088 | CGACGUGC CUGAUGAG GCCGUUAGGC CGAA IAGGUGAA | 8488 |
| 1603 | ACCUCUGC A CGUCGCAU | 1089 | AUGCGACG CUGAUGAG GCCGUUAGGC CGAA ICAGAGGU | 8489 |
| 1610 | CACGUCGC A UGGAGACC | 1090 | GGUCUCCA CUGAUGAG GCCGUUAGGC CGAA ICGACGUG | 8490 |
| 1618 | AUGGAGAC C ACCGUGAA | 1091 | UUCACGGU CUGAUGAG GCCGUUAGGC CGAA IUCUCCAU | 8491 |
| 1619 | UGGAGACC A CCGUGAAC | 1092 | GUUCACGG CUGAUGAG GCCGUUAGGC CGAA IGUCUCCA | 8492 |
| 1621 | GAGACCAC C GUGAACGC | 1093 | GCGUUCAC CUGAUGAG GCCGUUAGGC CGAA IUGGUCUC | 8493 |
| 1630 | GUGAACGC C CACAGGAA | 1094 | UUCCUGUG CUGAUGAG GCCGUUAGGC CGAA ICGUUCAC | 8494 |
| 1631 | UGAACGCC C ACAGGAAC | 1095 | GUUCCUGU CUGAUGAG GCCGUUAGGC CGAA IGCGUUCA | 8495 |
| 1632 | GAACGCCC A CAGGAACC | 1096 | GGUUCCUG CUGAUGAG GCCGUUAGGC CGAA IGGCGUUC | 8496 |
| 1634 | ACGCCCAC A GGAACCUG | 1097 | CAGGUUCC CUGAUGAG GCCGUUAGGC CGAA IUGGGCGU | 8497 |
| 1640 | ACAGGAAC C UGCCCAAG | 1098 | CUUGGGCA CUGAUGAG GCCGUUAGGC CGAA IUUCCUGU | 8498 |
| 1641 | CAGGAACC U GCCCAAGG | 1099 | CCUUGGGC CUGAUGAG GCCGUUAGGC CGAA IGUUCCUG | 8499 |
| 1644 | GAACCUGC C CAAGGUCU | 1100 | AGACCUUG CUGAUGAG GCCGUUAGGC CGAA ICAGGUUC | 8500 |
| 1645 | AACCUGCC C AAGGUCUU | 1101 | AAGACCUU CUGAUGAG GCCGUUAGGC CGAA IGCAGGUU | 8501 |
| 1646 | ACCUGCCC A AGGUCUUG | 1102 | CAAGACCU CUGAUGAG GCCGUUAGGC CGAA IGGCAGGU | 8502 |
| 1652 | CCAAGGUC U UGCAUAAG | 1103 | CUUAUGCA CUGAUGAG GCCGUUAGGC CGAA IACCUUGG | 8503 |
| 1656 | GGUCUUGC A UAAGAGGA | 1104 | UCCUCUUA CUGAUGAG GCCGUUAGGC CGAA ICAAGACC | 8504 |
| 1666 | AAGAGGAC U CUUGGACU | 1105 | AGUCCAAG CUGAUGAG GCCGUUAGGC CGAA IUCCUCUU | 8505 |
| 1668 | GAGGACUC U UGGACUUU | 1106 | AAAGUCCA CUGAUGAG GCCGUUAGGC CGAA IAGUCCUC | 8506 |
| 1674 | UCUUGGAC U UUCAGCAA | 1107 | UUGCUGAA CUGAUGAG GCCGUUAGGC CGAA IUCCAAGA | 8507 |
| 1678 | GGACUUUC A GCAAUGUC | 1108 | GACAUUGC CUGAUGAG GCCGUUAGGC CGAA IAAAGUCC | 8508 |
| 1681 | CUUUCAGC A AUGUCAAC | 1109 | GUUGACAU CUGAUGAG GCCGUUAGGC CGAA ICUGAAAG | 8509 |
| 1687 | GCAAUGUC A ACGACCGA | 1110 | UCGGUCGU CUGAUGAG GCCGUUAGGC CGAA IACAUUGC | 8510 |
| 1693 | UCAACGAC C GACCUUGA | 1111 | UCAAGGUC CUGAUGAG GCCGUUAGGC CGAA IUCGUUGA | 8511 |
| 1697 | CGACCGAC C UUGAGGCA | 1112 | UGCCUCAA CUGAUGAG GCCGUUAGGC CGAA IUCGGUCG | 8512 |
| 1698 | GACCGACC U UGAGGCAU | 1113 | AUGCCUCA CUGAUGAG GCCGUUAGGC CGAA IGUCGGUC | 8513 |
| 1705 | CUUGAGGC A UACUUCAA | 1114 | UUGAAGUA CUGAUGAG GCCGUUAGGC CGAA ICCUCAAG | 8514 |
| 1709 | AGGCAUAC U UCAAAGAC | 1115 | GUCUUUGA CUGAUGAG GCCGUUAGGC CGAA IUAUGCCU | 8515 |
| 1712 | CAUACUUC A AAGACUGU | 1116 | ACAGUCUU CUGAUGAG GCCGUUAGGC CGAA IAAGUAUG | 8516 |
| 1718 | UCAAAGAC U GUGUGUUU | 1117 | AAACACAC CUGAUGAG GCCGUUAGGC CGAA IUCUUUGA | 8517 |
| 1769 | UAAAGGUC U UUGUACUA | 1118 | UAGUACAA CUGAUGAG GCCGUUAGGC CGAA IACCUUUA | 8518 |
| 1776 | CUUUGUAC U AGGAGGCU | 1119 | AGCCUCCU CUGAUGAG GCCGUUAGGC CGAA IUACAAAG | 8519 |
| 1784 | UAGGAGGC U GUAGGCAU | 1120 | AUGCCUAC CUGAUGAG GCCGUUAGGC CGAA ICCUCCUA | 8520 |
| 1791 | CUGUAGGC A UAAAUUGG | 1121 | CCAAUUUA CUGAUGAG GCCGUUAGGC CGAA ICCUACAG | 8521 |
| 1807 | GUGUGUUC A CCAGCACC | 1122 | GGUGCUGG CUGAUGAG GCCGUUAGGC CGAA IAACACAC | 8522 |
| 1809 | GUGUUCAC C AGCACCAU | 1123 | AUGGUGCU CUGAUGAG GCCGUUAGGC CGAA IUGAACAC | 8523 |
| 1810 | UGUUCACC A GCACCAUG | 1124 | CAUGGUGC CUGAUGAG GCCGUUAGGC CGAA IGUGAACA | 8524 |
| 1813 | UCACCAGC A CCAUGCAA | 1125 | UUGCAUGG CUGAUGAG GCCGUUAGGC CGAA ICUGGUGA | 8525 |
| 1815 | ACCAGCAC C AUGCAACU | 1126 | AGUUGCAU CUGAUGAG GCCGUUAGGC CGAA IUGCUGGU | 8526 |
| 1816 | CCAGCACC A UGCAACUU | 1127 | AAGUUGCA CUGAUGAG GCCGUUAGGC CGAA IGUGCUGG | 8527 |
| 1820 | CACCAUGC A ACUUUUUC | 1128 | GAAAAAGU CUGAUGAG GCCGUUAGGC CGAA ICAUGGUG | 8528 |
| 1823 | CAUGCAAC U UUUUCACC | 1129 | GGUGAAAA CUGAUGAG GCCGUUAGGC CGAA IUUGCAUG | 8529 |
| 1829 | ACUUUUUC A CCUCUGCC | 1130 | GGCAGAGG CUGAUGAG GCCGUUAGGC CGAA IAAAAAGU | 8530 |
| 1831 | UUUUUCAC C UCUGCCUA | 1131 | UAGGCAGA CUGAUGAG GCCGUUAGGC CGAA IUGAAAAA | 8531 |
| 1832 | UUUUCACC U CUGCCUAA | 1132 | UUAGGCAG CUGAUGAG GCCGUUAGGC CGAA IGUGAAAA | 8532 |
| 1834 | UUCACCUC U GCCUAAUC | 1133 | GAUUAGGC CUGAUGAG GCCGUUAGGC CGAA IAGGUGAA | 8533 |
| 1837 | ACCUCUGC C UAAUCAUC | 1134 | GAUGAUUA CUGAUGAG GCCGUUAGGC CGAA ICAGAGGU | 8534 |
| 1838 | CCUCUGCC U AAUCAUCU | 1135 | AGAUGAUU CUGAUGAG GCCGUUAGGC CGAA IGCAGAGG | 8535 |
| 1843 | GCCUAAUC A UCUCAUGU | 1136 | ACAUGAGA CUGAUGAG GCCGUUAGGC CGAA IAUUAGGC | 8536 |
| 1846 | UAAUCAUC U CAUGUUCA | 1137 | UGAACAUG CUGAUGAG GCCGUUAGGC CGAA IAUGAUUA | 8537 |
| 1848 | AUCAUCUC A UGUUCAUG | 1138 | CAUGAACA CUGAUGAG GCCGUUAGGC CGAA IAGAUGAU | 8538 |
| 1854 | UCAUGUUC A UGUCCUAC | 1139 | GUAGGACA CUGAUGAG GCCGUUAGGC CGAA IAACAUGA | 8539 |
| 1859 | UUCAUGUC C UACUGUUC | 1140 | GAACAGUA CUGAUGAG GCCGUUAGGC CGAA IACAUGAA | 8540 |
| 1860 | UCAUGUCC U ACUGUUCA | 1141 | UGAACAGU CUGAUGAG GCCGUUAGGC CGAA IGACAUGA | 8541 |
| 1863 | UGUCCUAC U GUUCAAGC | 1142 | GCUUGAAC CUGAUGAG GCCGUUAGGC CGAA IUAGGACA | 8542 |
| 1868 | UACUGUUC A AGCCUCCA | 1143 | UGGAGGCU CUGAUGAG GCCGUUAGGC CGAA IAACAGUA | 8543 |
| 1872 | GUUCAAGC C UCCAAGCU | 1144 | AGCUUGGA CUGAUGAG GCCGUUAGGC CGAA ICUUGAAC | 8544 |
| 1873 | UUCAAGCC U CCAAGCUG | 1145 | CAGCUUGG CUGAUGAG GCCGUUAGGC CGAA IGCUUGAA | 8545 |
| 1875 | CAAGCCUC C AAGCUGUG | 1146 | CACAGCUU CUGAUGAG GCCGUUAGGC CGAA IAGGCUUG | 8546 |
| 1876 | AAGCCUCC A AGCUGUGC | 1147 | GCACAGCU CUGAUGAG GCCGUUAGGC CGAA IGAGGCUU | 8547 |
| 1880 | CUCCAAGC U GUGCCUUG | 1148 | CAAGGCAC CUGAUGAG GCCGUUAGGC CGAA ICUUGGAG | 8548 |
| 1885 | AGCUGUGC C UUGGGUGG | 1149 | CCACCCAA CUGAUGAG GCCGUUAGGC CGAA ICACAGCU | 8549 |
| 1886 | GCUGUGCC U UGGGUGGC | 1150 | GCCACCCA CUGAUGAG GCCGUUAGGC CGAA IGCACAGC | 8550 |
| 1895 | UGGGUGGC U UUGGGGCA | 1151 | UGCCCCAA CUGAUGAG GCCGUUAGGC CGAA ICCACCCA | 8551 |
| 1903 | UUUGGGGC A UGGACAUU | 1152 | AAUGUCCA CUGAUGAG GCCGUUAGGC CGAA ICCCCAAA | 8552 |
| 1909 | GCAUGGAC A UUGACCCG | 1153 | CGGGUCAA CUGAUGAG GCCGUUAGGC CGAA IUCCAUGC | 8553 |
| 1915 | ACAUUGAC C CGUAUAAA | 1154 | UUUAUACG CUGAUGAG GCCGUUAGGC CGAA IUCAAUGU | 8554 |
| 1916 | CAUUGACC C GUAUAAAG | 1155 | CUUUAUAC CUGAUGAG GCCGUUAGGC CGAA IGUCAAUG | 8555 |
| 1935 | UUUGGAGC U UCUGUGGA | 1156 | UCCACAGA CUGAUGAG GCCGUUAGGC CGAA ICUCCAAA | 8556 |
| 1938 | GGAGCUUC U GUGGAGUU | 1157 | AACUCCAC CUGAUGAG GCCGUUAGGC CGAA IAAGCUCC | 8557 |
| 1949 | GGAGUUAC U CUCUUUUU | 1158 | AAAAAGAG CUGAUGAG GCCGUUAGGC CGAA IUAACUCC | 8558 |
| 1951 | AGUUACUC U CUUUUUUG | 1159 | CAAAAAAG CUGAUGAG GCCGUUAGGC CGAA IAGUAACU | 8559 |
| 1953 | UUACUCUC U UUUUUGCC | 1160 | GGCAAAAA CUGAUGAG GCCGUUAGGC CGAA IAGAGUAA | 8560 |
| 1961 | UUUUUUGC C UUCUGACU | 1161 | AGUCAGAA CUGAUGAG GCCGUUAGGC CGAA ICAAAAAA | 8561 |
| 1962 | UUUUUGCC U UCUGACUU | 1162 | AAGUCAGA CUGAUGAG GCCGUUAGGC CGAA IGCAAAAA | 8562 |
| 1965 | UUGCCUUC U GACUUCUU | 1163 | AAGAAGUC CUGAUGAG GCCGUUAGGC CGAA IAAGGCAA | 8563 |
| 1969 | CUUCUGAC U UCUUUCCU | 1164 | AGGAAAGA CUGAUGAG GCCGUUAGGC CGAA IUCAGAAG | 8564 |
| 1972 | CUGACUUC U UUCCUUCU | 1165 | AGAAGGAA CUGAUGAG GCCGUUAGGC CGAA IAAGUCAG | 8565 |
| 1976 | CUUCUUUC C UUCUAUUC | 1166 | GAAUAGAA CUGAUGAG GCCGUUAGGC CGAA IAAAGAAG | 8566 |
| 1977 | UUCUUUCC U UCUAUUCG | 1167 | CGAAUAGA CUGAUGAG GCCGUUAGGC CGAA IGAAAGAA | 8567 |
| 1980 | UUUCCUUC U AUUCGAGA | 1168 | UCUCGAAU CUGAUGAG GCCGUUAGGC CGAA IAAGGAAA | 8568 |
| 1991 | UCGAGAUC U CCUCGACA | 1169 | UGUCGAGG CUGAUGAG GCCGUUAGGC CGAA IAUCUCGA | 8569 |
| 1993 | GAGAUCUC C UCGACACC | 1170 | GGUGUCGA CUGAUGAG GCCGUUAGGC CGAA IAGAUCUC | 8570 |
| 1994 | AGAUCUCC U CGACACCG | 1171 | CGGUGUCG CUGAUGAG GCCGUUAGGC CGAA IGAGAUCU | 8571 |
| 1999 | UCCUCGAC A CCGCCUCU | 1172 | AGAGGCGG CUGAUGAG GCCGUUAGGC CGAA IUCGAGGA | 8572 |
| 2001 | CUCGACAC C GCCUCUGC | 1173 | GCAGAGGC CUGAUGAG GCCGUUAGGC CGAA IUGUCGAG | 8573 |
| 2004 | GACACCGC C UCUGCUCU | 1174 | AGAGCAGA CUGAUGAG GCCGUUAGGC CGAA ICGGUGUC | 8574 |
| 2005 | ACACCGCC U CUGCUCUG | 1175 | CAGAGCAG CUGAUGAG GCCGUUAGGC CGAA IGCGGUGU | 8575 |
| 2007 | ACCGCCUC U GCUCUGUA | 1176 | UACAGAGC CUGAUGAG GCCGUUAGGC CGAA IAGGCGGU | 8576 |
| 2010 | GCCUCUGC U CUGUAUCG | 1177 | CGAUACAG CUGAUGAG GCCGUUAGGC CGAA ICAGAGGC | 8577 |
| 2012 | CUCUGCUC U GUAUCGGG | 1178 | CCCGAUAC CUGAUGAG GCCGUUAGGC CGAA IAGCAGAG | 8578 |
| 2025 | CGGGGGGC C UUAGAGUC | 1179 | GACUCUAA CUGAUGAG GCCGUUAGGC CGAA ICCCCCCG | 8579 |
| 2026 | GGGGGGCC U UAGAGUCU | 1180 | AGACUCUA CUGAUGAG GCCGUUAGGC CGAA IGCCCCCC | 8580 |
| 2034 | UUAGAGUC U CCGGAACA | 1181 | UGUUCCGG CUGAUGAG GCCGUUAGGC CGAA IACUCUAA | 8581 |
| 2036 | AGAGUCUC C GGAACAUU | 1182 | AAUGUUCC CUGAUGAG GCCGUUAGGC CGAA IAGACUCU | 8582 |
| 2042 | UCCGGAAC A UUGUUCAC | 1183 | GUGAACAA CUGAUGAG GCCGUUAGGC CGAA IUUCCGGA | 8583 |
| 2049 | CAUUGUUC A CCUCACCA | 1184 | UGGUGAGG CUGAUGAG GCCGUUAGGC CGAA IAACAAUG | 8584 |
| 2051 | UUGUUCAC C UCACCAUA | 1185 | UAUGGUGA CUGAUGAG GCCGUUAGGC CGAA IUGAACAA | 8585 |
| 2052 | UGUUCACC U CACCAUAC | 1186 | GUAUGGUG CUGAUGAG GCCGUUAGGC CGAA IGUGAACA | 8586 |
| 2054 | UUCACCUC A CCAUACGG | 1187 | CCGUAUGG CUGAUGAG GCCGUUAGGC CGAA IAGGUGAA | 8587 |
| 2056 | CACCUCAC C AUACGGCA | 1188 | UGCCGUAU CUGAUGAG GCCGUUAGGC CGAA IUGAGGUG | 8588 |
| 2057 | ACCUCACC A UACGGCAC | 1189 | GUGCCGUA CUGAUGAG GCCGUUAGGC CGAA IGUGAGGU | 8589 |
| 2064 | CAUACGGC A CUCAGGCA | 1190 | UGCCUGAG CUGAUGAG GCCGUUAGGC CGAA ICCGUAUG | 8590 |
| 2066 | UACGGCAC U CAGGCAAG | 1191 | CUUGCCUG CUGAUGAG GCCGUUAGGC CGAA IUGCCGUA | 8591 |
| 2068 | CGGCACUC A GGCAAGCU | 1192 | AGCUUGCC CUGAUGAG GCCGUUAGGC CGAA IAGUGCCG | 8592 |
| 2072 | ACUCAGGC A AGCUAUUC | 1193 | GAAUAGCU CUGAUGAG GCCGUUAGGC CGAA ICCUGAGU | 8593 |
| 2076 | AGGCAAGC U AUUCUGUG | 1194 | CACAGAAU CUGAUGAG GCCGUUAGGC CGAA ICUUGCCU | 8594 |
| 2081 | AGCUAUUC U GUGUUGGG | 1195 | CCCAACAC CUGAUGAG GCCGUUAGGC CGAA IAAUAGCU | 8595 |
| 2105 | GAUGAAUC U AGCCACCU | 1196 | AGGUGGCU CUGAUGAG GCCGUUAGGC CGAA IAUUCAUC | 8596 |
| 2109 | AAUCUAGC C ACCUGGGU | 1197 | ACCCAGGU CUGAUGAG GCCGUUAGGC CGAA ICUAGAUU | 8597 |
| 2110 | AUCUAGCC A CCUGGGUG | 1198 | CACCCAGG CUGAUGAG GCCGUUAGGC CGAA IGCUAGAU | 8598 |
| 2112 | CUAGCCAC C UGGGUGGG | 1199 | CCCACCCA CUGAUGAG GCCGUUAGGC CGAA IUGGCUAG | 8599 |
| 2113 | UAGCCACC U GGGUGGGA | 1200 | UCCCACCC CUGAUGAG GCCGUUAGGC CGAA IGUGGCUA | 8600 |
| 2138 | GGAAGAUC C AGCAUCCA | 1201 | UGGAUGCU CUGAUGAG GCCGUUAGGC CGAA IAUCUUCC | 8601 |
| 2139 | GAAGAUCC A GCAUCCAG | 1202 | CUGGAUGC CUGAUGAG GCCGUUAGGC CGAA IGAUCUUC | 8602 |
| 2142 | GAUCCAGC A UCCAGGGA | 1203 | UCCCUGGA CUGAUGAG GCCGUUAGGC CGAA ICUGGAUC | 8603 |
| 2145 | CCAGCAUC C AGGGAAUU | 1204 | AAUUCCCU CUGAUGAG GCCGUUAGGC CGAA IAUGCUGG | 8604 |
| 2146 | CAGCAUCC A GGGAAUUA | 1205 | UAAUUCCC CUGAUGAG GCCGUUAGGC CGAA IGAUGCUG | 8605 |
| 2161 | UAGUAGUC A GCUAUGUC | 1206 | GACAUAGC CUGAUGAG GCCGUUAGGC CGAA IACUACUA | 8606 |
| 2164 | UAGUCAGC U AUGUCAAC | 1207 | GUUGACAU CUGAUGAG GCCGUUAGGC CGAA ICUGACUA | 8607 |
| 2170 | GCUAUGUC A ACGUUAAU | 1208 | AUUAACGU CUGAUGAG GCCGUUAGGC CGAA IACAUAGC | 8608 |
| 2185 | AUAUGGGC C UAAAAAUC | 1209 | GAUUUUUA CUGAUGAG GCCGUUAGGC CGAA ICCCAUAU | 8609 |
| 2186 | UAUGGGCC U AAAAAUCA | 1210 | UGAUUUUU CUGAUGAG GCCGUUAGGC CGAA IGCCCAUA | 8610 |
| 2194 | UAAAAAUC A GACAACUA | 1211 | UAGUUGUC CUGAUGAG GCCGUUAGGC CGAA IAUUUUUA | 8611 |
| 2198 | AAUCAGAC A ACUAUUGU | 1212 | ACAAUAGU CUGAUGAG GCCGUUAGGC CGAA IUCUGAUU | 8612 |
| 2201 | CAGACAAC U AUUGUGGU | 1213 | ACCACAAU CUGAUGAG GCCGUUAGGC CGAA IUUGUCUG | 8613 |
| 2213 | GUGGUUUC A CAUUUCCU | 1214 | AGGAAAUG CUGAUGAG GCCGUUAGGC CGAA IAAACCAC | 8614 |
| 2215 | GGUUUCAC A UUUCCUGU | 1215 | ACAGGAAA CUGAUGAG GCCGUUAGGC CGAA IUGAAACC | 8615 |
| 2220 | CACAUUUC C UGUCUUAC | 1216 | GUAAGACA CUGAUGAG GCCGUUAGGC CGAA IAAAUGUG | 8616 |
| 2221 | ACAUUUCC U GUCUUACU | 1217 | AGUAAGAC CUGAUGAG GCCGUUAGGC CGAA IGAAAUGU | 8617 |
| 2225 | UUCCUGUC U UACUUUUG | 1218 | CAAAAGUA CUGAUGAG GCCGUUAGGC CGAA IACAGGAA | 8618 |
| 2229 | UGUCUUAC U UUUGGGCG | 1219 | CGCCCAAA CUGAUGAG GCCGUUAGGC CGAA IUAAGACA | 8619 |
| 2244 | CGAGAAAC U GUUCUUGA | 1220 | UCAAGAAC CUGAUGAG GCCGUUAGGC CGAA IUUUCUCG | 8620 |
| 2249 | AACUGUUC U UGAAUAUU | 1221 | AAUAUUCA CUGAUGAG GCCGUUAGGC CGAA IAACAGUU | 8621 |
| 2265 | UUGGUGUC U UUUGGAGU | 1222 | ACUCCAAA CUGAUGAG GCCGUUAGGC CGAA IACACCAA | 8622 |
| 2284 | GGAUUCGC A CUCCUCCU | 1223 | AGGAGGAG CUGAUGAG GCCGUUAGGC CGAA ICGAAUCC | 8623 |
| 2286 | AUUCGCAC U CCUCCUGC | 1224 | GCAGGAGG CUGAUGAG GCCGUUAGGC CGAA IUGCGAAU | 8624 |
| 2288 | UCGCACUC C UCCUGCAU | 1225 | AUGCAGGA CUGAUGAG GCCGUUAGGC CGAA IAGUGCGA | 8625 |
| 2289 | CGCACUCC U CCUGCAUA | 1226 | UAUGCAGG CUGAUGAG GCCGUUAGGC CGAA IGAGUGCG | 8626 |
| 2291 | CACUCCUC C UGCAUAUA | 1227 | UAUAUGCA CUGAUGAG GCCGUUAGGC CGAA IAGGAGUG | 8627 |
| 2292 | ACUCCUCC U GCAUAUAG | 1228 | CUAUAUGC CUGAUGAG GCCGUUAGGC CGAA IGAGGAGU | 8628 |
| 2295 | CCUCCUGC A UAUAGACC | 1229 | GGUCUAUA CUGAUGAG GCCGUUAGGC CGAA ICAGGAGG | 8629 |
| 2303 | AUAUAGAC C ACCAAAUG | 1230 | CAUUUGGU CUGAUGAG GCCGUUAGGC CGAA IUCUAUAU | 8630 |
| 2304 | UAUAGACC A CCAAAUGC | 1231 | GCAUUUGG CUGAUGAG GCCGUUAGGC CGAA IGUCUAUA | 8631 |
| 2306 | UAGACCAC C AAAUGCCC | 1232 | GGGCAUUU CUGAUGAG GCCGUUAGGC CGAA IUGGUCUA | 8632 |
| 2307 | AGACCACC A AAUGCCCC | 1233 | GGGGCAUU CUGAUGAG GCCGUUAGGC CGAA IGUGGUCU | 8633 |
| 2313 | CCAAAUGC C CCUAUCUU | 1234 | AAGAUAGG CUGAUGAG GCCGUUAGGC CGAA ICAUUUGG | 8634 |
| 2314 | CAAAUGCC C CUAUCUUA | 1235 | UAAGAUAG CUGAUGAG GCCGUUAGGC CGAA IGCAUUUG | 8635 |
| 2315 | AAAUGCCC C UAUCUUAU | 1236 | AUAAGAUA CUGAUGAG GCCGUUAGGC CGAA IGGCAUUU | 8636 |
| 2316 | AAUGCCCC U AUCUUAUC | 1237 | GAUAAGAU CUGAUGAG GCCGUUAGGC CGAA IGGGCAUU | 8637 |
| 2320 | CCCCUAUC U UAUCAACA | 1238 | UGUUGAUA CUGAUGAG GCCGUUAGGC CGAA IAUAGGGG | 8638 |
| 2325 | AUCUUAUC A ACACUUCC | 1239 | GGAAGUGU CUGAUGAG GCCGUUAGGC CGAA IAUAAGAU | 8639 |
| 2328 | UUAUCAAC A CUUCCGGA | 1240 | UCCGGAAG CUGAUGAG GCCGUUAGGC CGAA IUUGAUAA | 8640 |
| 2330 | AUCAACAC U UCCGGAAA | 1241 | UUUCCGGA CUGAUGAG GCCGUUAGGC CGAA IUGUUGAU | 8641 |
| 2333 | AACACUUC C GGAAACUA | 1242 | UAGUUUCC CUGAUGAG GCCGUUAGGC CGAA IAAGUGUU | 8642 |
| 2340 | CCGGAAAC U ACUGUUGU | 1243 | ACAACAGU CUGAUGAG GCCGUUAGGC CGAA IUUUCCGG | 8643 |
| 2343 | GAAACUAC U GUUGUUAG | 1244 | CUAACAAC CUGAUGAG GCCGUUAGGC CGAA IUAGUUUC | 8644 |
| 2362 | GAAGAGGC A GGUCCCCU | 1245 | AGGGGACC CUGAUGAG GCCGUUAGGC CGAA ICCUCUUC | 8645 |
| 2367 | GGCAGGUC C CCUAGAAG | 1246 | CUUCUAGG CUGAUGAG GCCGUUAGGC CGAA IACCUGCC | 8646 |
| 2368 | GCAGGUCC C CUAGAAGA | 1247 | UCUUCUAG CUGAUGAG GCCGUUAGGC CGAA IGACCUGC | 8647 |
| 2369 | CAGGUCCC C UAGAAGAA | 1248 | UUCUUCUA CUGAUGAG GCCGUUAGGC CGAA IGGACCUG | 8648 |
| 2370 | AGGUCCCC U AGAAGAAG | 1249 | CUUCUUCU CUGAUGAG GCCGUUAGGC CGAA IGGGACCU | 8649 |
| 2382 | AGAAGAAC U CCCUCGCC | 1250 | GGCGAGGG CUGAUGAG GCCGUUAGGC CGAA IUUCUUCU | 8650 |
| 2384 | AAGAACUC C CUCGCCUC | 1251 | GAGGCGAG CUGAUGAG GCCGUUAGGC CGAA IAGUUCUU | 8651 |
| 2385 | AGAACUCC C UCGCCUCG | 1252 | CGAGGCGA CUGAUGAG GCCGUUAGGC CGAA IGAGUUCU | 8652 |
| 2386 | GAACUCCC U CGCCUCGC | 1253 | GCGAGGCG CUGAUGAG GCCGUUAGGC CGAA IGGAGUUC | 8653 |
| 2390 | UCCCUCGC C UCGCAGAC | 1254 | GUCUGCGA CUGAUGAG GCCGUUAGGC CGAA ICGAGGGA | 8654 |
| 2391 | CCCUCGCC U CGCAGACG | 1255 | CGUCUGCG CUGAUGAG GCCGUUAGGC CGAA IGCGAGGG | 8655 |
| 2395 | CGCCUCGC A GACGAAGG | 1256 | CCUUCGUC CUGAUGAG GCCGUUAGGC CGAA ICGAGGCG | 8656 |
| 2406 | CGAAGGUC U CAAUCGCC | 1257 | GGCGAUUG CUGAUGAG GCCGUUAGGC CGAA IACCUUCG | 8657 |
| 2408 | AAGGUCUC A AUCGCCGC | 1258 | GCGGCGAU CUGAUGAG GCCGUUAGGC CGAA IAGACCUU | 8658 |
| 2414 | UCAAUCGC C GCGUCGCA | 1259 | UGCGACGC CUGAUGAG GCCGUUAGGC CGAA ICGAUUGA | 8659 |
| 2422 | CGCGUCGC A GAAGAUCU | 1260 | AGAUCUUC CUGAUGAG GCCGUUAGGC CGAA ICGACGCG | 8660 |
| 2430 | AGAAGAUC U CAAUCUCG | 1261 | CGAGAUUG CUGAUGAG GCCGUUAGGC CGAA IAUCUUCU | 8661 |
| 2432 | AAGAUCUC A AUCUCGGG | 1262 | CCCGAGAU CUGAUGAG GCCGUUAGGC CGAA IAGAUCUU | 8662 |
| 2436 | UCUCAAUC U CGGGAAUC | 1263 | GAUUCCCG CUGAUGAG GCCGUUAGGC CGAA IAUUGAGA | 8663 |
| 2445 | CGGGAAUC U CAAUGUUA | 1264 | UAACAUUG CUGAUGAG GCCGUUAGGC CGAA IAUUCCCG | 8664 |
| 2447 | GGAAUCUC A AUGUUAGU | 1265 | ACUAACAU CUGAUGAG GCCGUUAGGC CGAA IAGAUUCC | 8665 |
| 2460 | UAGUAUUC C UUGGACAC | 1266 | GUGUCCAA CUGAUGAG GCCGUUAGGC CGAA IAAUACUA | 8666 |
| 2461 | AGUAUUCC U UGGACACA | 1267 | UGUGUCCA CUGAUGAG GCCGUUAGGC CGAA IGAAUACU | 8667 |
| 2467 | CCUUGGAC A CAUAAGGU | 1268 | ACCUUAUG CUGAUGAG GCCGUUAGGC CGAA IUCCAAGG | 8668 |
| 2469 | UUGGACAC A UAAGGUGG | 1269 | CCACCUUA CUGAUGAG GCCGUUAGGC CGAA IUGUCCAA | 8669 |
| 2483 | UGGGAAAC U UUACGGGG | 1270 | CCCCGUAA CUGAUGAG GCCGUUAGGC CGAA IUUUCCCA | 8670 |
| 2493 | UACGGGGC U UUAUUCUU | 1271 | AAGAAUAA CUGAUGAG GCCGUUAGGC CGAA ICCCCGUA | 8671 |
| 2500 | CUUUAUUC U UCUACGGU | 1272 | ACCGUAGA CUGAUGAG GCCGUUAGGC CGAA IAAUAAAG | 8672 |
| 2503 | UAUUCUUC U ACGGUACC | 1273 | GGUACCGU CUGAUGAG GCCGUUAGGC CGAA IAAGAAUA | 8673 |
| 2511 | UACGGUAC C UUGCUUUA | 1274 | UAAAGCAA CUGAUGAG GCCGUUAGGC CGAA IUACCGUA | 8674 |
| 2512 | ACGGUACC U UGCUUUAA | 1275 | UUAAAGCA CUGAUGAG GCCGUUAGGC CGAA IGUACCGU | 8675 |
| 2516 | UACCUUGC U UUAAUCCU | 1276 | AGGAUUAA CUGAUGAG GCCGUUAGGC CGAA ICAAGGUA | 8676 |
| 2523 | CUUUAAUC C UAAAUGGC | 1277 | GCCAUUUA CUGAUGAG GCCGUUAGGC CGAA IAUUAAAG | 8677 |
| 2524 | UUUAAUCC U AAAUGGCA | 1278 | UGCCAUUU CUGAUGAG GCCGUUAGGC CGAA IGAUUAAA | 8678 |
| 2532 | UAAAUGGC A AACUCCUU | 1279 | AAGGAGUU CUGAUGAG GCCGUUAGGC CGAA ICCAUUUA | 8679 |
| 2536 | UGGCAAAC U CCUUCUUU | 1280 | AAAGAAGG CUGAUGAG GCCGUUAGGC CGAA IUUUGCCA | 8680 |
| 2538 | GCAAACUC C UUCUUUUC | 1281 | GAAAAGAA CUGAUGAG GCCGUUAGGC CGAA IAGUUUGC | 8681 |
| 2539 | CAAACUCC U UCUUUUCC | 1282 | GGAAAAGA CUGAUGAG GCCGUUAGGC CGAA IGAGUUUG | 8682 |
| 2542 | ACUCCUUC U UUUCCUGA | 1283 | UCAGGAAA CUGAUGAG GCCGUUAGGC CGAA IAAGGAGU | 8683 |
| 2547 | UUCUUUUC C UGACAUUC | 1284 | GAAUGUCA CUGAUGAG GCCGUUAGGC CGAA IAAAAGAA | 8684 |
| 2548 | UCUUUUCC U GACAUUCA | 1285 | UGAAUGUC CUGAUGAG GCCGUUAGGC CGAA IGAAAAGA | 8685 |
| 2552 | UUCCUGAC A UUCAUUUG | 1286 | CAAAUGAA CUGAUGAG GCCGUUAGGC CGAA IUCAGGAA | 8686 |
| 2556 | UGACAUUC A UUUGCAGG | 1287 | CCUGCAAA CUGAUGAG GCCGUUAGGC CGAA IAAUGUCA | 8687 |
| 2562 | UCAUUUGC A GGAGGACA | 1288 | UGUCCUCC CUGAUGAG GCCGUUAGGC CGAA ICAAAUGA | 8688 |
| 2570 | AGGAGGAC A UUGUUGAU | 1289 | AUCAACAA CUGAUGAG GCCGUUAGGC CGAA IUCCUCCU | 8689 |
| 2589 | AUGUAAGC A AUUUGUGG | 1290 | CCACAAAU CUGAUGAG GCCGUUAGGC CGAA ICUUACAU | 8690 |
| 2601 | UGUGGGGC C CCUUACAG | 1291 | CUGUAAGG CUGAUGAG GCCGUUAGGC CGAA ICCCCACA | 8691 |
| 2602 | GUGGGGCC C CUUACAGU | 1292 | ACUGUAAG CUGAUGAG GCCGUUAGGC CGAA IGCCCCAC | 8692 |
| 2603 | UGGGGCCC C UUACAGUA | 1293 | UACUGUAA CUGAUGAG GCCGUUAGGC CGAA IGGCCCCA | 8693 |
| 2604 | GGGGCCCC U UACAGUAA | 1294 | UUACUGUA CUGAUGAG GCCGUUAGGC CGAA IGGGCCCC | 8694 |
| 2608 | CCCCUUAC A GUAAAUGA | 1295 | UCAUUUAC CUGAUGAG GCCGUUAGGC CGAA IUAAGGGG | 8695 |
| 2621 | AUGAAAAC A GGAGACUU | 1296 | AAGUCUCC CUGAUGAG GCCGUUAGGC CGAA IUUUUCAU | 8696 |
| 2628 | CAGGAGAC U UAAAUUAA | 1297 | UUAAUUUA CUGAUGAG GCCGUUAGGC CGAA IUCUCCUG | 8697 |
| 2638 | AAAUUAAC U AUGCCUGC | 1298 | GCAGGCAU CUGAUGAG GCCGUUAGGC CGAA IUUAAUUU | 8698 |
| 2643 | AACUAUGC C UGCUAGGU | 1299 | ACCUAGCA CUGAUGAG GCCGUUAGGC CGAA ICAUAGUU | 8699 |
| 2644 | ACUAUGCC U GCUAGGUU | 1300 | AACCUAGC CUGAUGAG GCCGUUAGGC CGAA IGCAUAGU | 8700 |
| 2647 | AUGCCUGC U AGGUUUUA | 1301 | UAAAACCU CUGAUGAG GCCGUUAGGC CGAA ICAGGCAU | 8701 |
| 2658 | GUUUUAUC C CAAUGUUA | 1302 | UAACAUUG CUGAUGAG GCCGUUAGGC CGAA IAUAAAAC | 8702 |
| 2659 | UUUUAUCC C AAUGUUAC | 1303 | GUAACAUU CUGAUGAG GCCGUUAGGC CGAA IGAUAAAA | 8703 |
| 2660 | UUUAUCCC A AUGUUACU | 1304 | AGUAACAU CUGAUGAG GCCGUUAGGC CGAA IGGAUAAA | 8704 |
| 2668 | AAUGUUAC U AAAUAUUU | 1305 | AAAUAUUU CUGAUGAG GCCGUUAGGC CGAA IUAACAUU | 8705 |
| 2679 | AUAUUUGC C CUUAGAUA | 1306 | UAUCUAAG CUGAUGAG GCCGUUAGGC CGAA ICAAAUAU | 8706 |
| 2680 | UAUUUGCC C UUAGAUAA | 1307 | UUAUCUAA CUGAUGAG GCCGUUAGGC CGAA IGCAAAUA | 8707 |
| 2681 | AUUUGCCC U UAGAUAAA | 1308 | UUUAUCUA CUGAUGAG GCCGUUAGGC CGAA IGGCAAAU | 8708 |
| 2696 | AAGGGAUC A AACCGUAU | 1309 | AUACGGUU CUGAUGAG GCCGUUAGGC CGAA IAUCCCUU | 8709 |
| 2700 | GAUCAAAC C GUAUUAUC | 1310 | GAUAAUAC CUGAUGAG GCCGUUAGGC CGAA IUUUGAUC | 8710 |
| 2709 | GUAUUAUC C AGAGUAUG | 1311 | CAUACUCU CUGAUGAG GCCGUUAGGC CGAA IAUAAUAC | 8711 |
| 2710 | UAUUAUCC A GAGUAUGU | 1312 | ACAUACUC CUGAUGAG GCCGUUAGGC CGAA IGAUAAUA | 8712 |
| 2727 | AGUUAAUC A UUACUUCC | 1313 | GGAAGUAA CUGAUGAG GCCGUUAGGC CGAA IAUUAACU | 8713 |
| 2732 | AUCAUUAC U UCCAGACG | 1314 | CGUCUGGA CUGAUGAG GCCGUUAGGC CGAA IUAAUGAU | 8714 |
| 2735 | AUUACUUC C AGACGCGA | 1315 | UCGCGUCU CUGAUGAG GCCGUUAGGC CGAA IAAGUAAU | 8715 |
| 2736 | UUACUUCC A GACGCGAC | 1316 | GUCGCGUC CUGAUGAG GCCGUUAGGC CGAA IGAAGUAA | 8716 |
| 2745 | GACGCGAC A UUAUUUAC | 1317 | GUAAAUAA CUGAUGAG GCCGUUAGGC CGAA IUCGCGUC | 8717 |
| 2754 | UUAUUUAC A CACUCUUU | 1318 | AAAGAGUG CUGAUGAG GCCGUUAGGC CGAA IUAAAUAA | 8718 |
| 2756 | AUUUACAC A CUCUUUGG | 1319 | CCAAAGAG CUGAUGAG GCCGUUAGGC CGAA IUGUAAAU | 8719 |
| 2758 | UUACACAC U CUUUGGAA | 1320 | UUCCAAAG CUGAUGAG GCCGUUAGGC CGAA IUGUGUAA | 8720 |
| 2760 | ACACACUC U UUGGAAGG | 1321 | CCUUCCAA CUGAUGAG GCCGUUAGGC CGAA IAGUGUGU | 8721 |
| 2777 | CGGGGAUC U UAUAUAAA | 1322 | UUUAUAUA CUGAUGAG GCCGUUAGGC CGAA IAUCCCCG | 8722 |
| 2794 | AGAGAGUC C ACACGUAG | 1323 | CUACGUGU CUGAUGAG GCCGUUAGGC CGAA IACUCUCU | 8723 |
| 2795 | GAGAGUCC A CACGUAGC | 1324 | GCUACGUG CUGAUGAG GCCGUUAGGC CGAA IGACUCUC | 8724 |
| 2797 | GAGUCCAC A CGUAGCGC | 1325 | GCGCUACG CUGAUGAG GCCGUUAGGC CGAA IUGGACUC | 8725 |
| 2806 | CGUAGCGC C UCAUUUUG | 1326 | CAAAAUGA CUGAUGAG GCCGUUAGGC CGAA ICGCUACG | 8726 |
| 2807 | GUAGCGCC U CAUUUUGC | 1327 | GCAAAAUG CUGAUGAG GCCGUUAGGC CGAA IGCGCUAC | 8727 |
| 2809 | AGCGCCUC A UUUUGCGG | 1328 | CCGCAAAA CUGAUGAG GCCGUUAGGC CGAA IAGGCGCU | 8728 |
| 2821 | UGCGGGUC A CCAUAUUC | 1329 | GAAUAUGG CUGAUGAG GCCGUUAGGC CGAA IACCCGCA | 8729 |
| 2823 | CGGGUCAC C AUAUUCUU | 1330 | AAGAAUAU CUGAUGAG GCCGUUAGGC CGAA IUGACCCG | 8730 |
| 2824 | GGGUCACC A UAUUCUUG | 1331 | CAAGAAUA CUGAUGAG GCCGUUAGGC CGAA IGUGACCC | 8731 |
| 2830 | CCAUAUUC U UGGGAACA | 1332 | UGUUCCCA CUGAUGAG GCCGUUAGGC CGAA IAAUAUGG | 8732 |
| 2838 | UUGGGAAC A AGAUCUAC | 1333 | GUAGAUCU CUGAUGAG GCCGUUAGGC CGAA IUUCCCAA | 8733 |
| 2844 | ACAAGAUC U ACAGCAUG | 1334 | CAUGCUGU CUGAUGAG GCCGUUAGGC CGAA IAUCUUGU | 8734 |
| 2847 | AGAUCUAC A GCAUGGGA | 1335 | UCCCAUGC CUGAUGAG GCCGUUAGGC CGAA IUAGAUCU | 8735 |
| 2850 | UCUACAGC A UGGGAGGU | 1336 | ACCUCCCA CUGAUGAG GCCGUUAGGC CGAA ICUGUAGA | 8736 |
| 2864 | GGUUGGUC U UCCAAACC | 1337 | GGUUUGGA CUGAUGAG GCCGUUAGGC CGAA IACCAACC | 8737 |
| 2867 | UGGUCUUC C AAACCUCG | 1338 | CGAGGUUU CUGAUGAG GCCGUUAGGC CGAA IAAGACCA | 8738 |
| 2868 | GGUCUUCC A AACCUCGA | 1339 | UCGAGGUU CUGAUGAG GCCGUUAGGC CGAA IGAAGACC | 8739 |
| 2872 | UUCCAAAC C UCGAAAAG | 1340 | CUUUUCGA CUGAUGAG GCCGUUAGGC CGAA IUUUGGAA | 8740 |
| 2873 | UCCAAACC U CGAAAAGG | 1341 | CCUUUUCG CUGAUGAG GCCGUUAGGC CGAA IGUUUGGA | 8741 |
| 2883 | GAAAAGGC A UGGGGACA | 1342 | UGUCCCCA CUGAUGAG GCCGUUAGGC CGAA ICCUUUUC | 8742 |
| 2891 | AUGGGGAC A AAUCUUUC | 1343 | GAAAGAUU CUGAUGAG GCCGUUAGGC CGAA IUCCCCAU | 8743 |
| 2896 | GACAAAUC U UUCUGUCC | 1344 | GGACAGAA CUGAUGAG GCCGUUAGGC CGAA IAUUUGUC | 8744 |
| 2900 | AAUCUUUC U GUCCCCAA | 1345 | UUGGGGAC CUGAUGAG GCCGUUAGGC CGAA IAAAGAUU | 8745 |
| 2904 | UUUCUGUC C CCAAUCCC | 1346 | GGGAUUGG CUGAUGAG GCCGUUAGGC CGAA IACAGAAA | 8746 |
| 2905 | UUCUGUCC C CAAUCCCC | 1347 | GGGGAUUG CUGAUGAG GCCGUUAGGC CGAA IGACAGAA | 8747 |
| 2906 | UCUGUCCC C AAUCCCCU | 1348 | AGGGGAUU CUGAUGAG GCCGUUAGGC CGAA IGGACAGA | 8748 |
| 2907 | CUGUCCCC A AUCCCCUG | 1349 | CAGGGGAU CUGAUGAG GCCGUUAGGC CGAA IGGGACAG | 8749 |
| 2911 | CCCCAAUC C CCUGGGAU | 1350 | AUCCCAGG CUGAUGAG GCCGUUAGGC CGAA IAUUGGGG | 8750 |
| 2912 | CCCAAUCC C CUGGGAUU | 1351 | AAUCCCAG CUGAUGAG GCCGUUAGGC CGAA IGAUUGGG | 8751 |
| 2913 | CCAAUCCC C UGGGAUUC | 1352 | GAAUCCCA CUGAUGAG GCCGUUAGGC CGAA IGGAUUGG | 8752 |
| 2914 | CAAUCCCC U GGGAUUCU | 1353 | AGAAUCCC CUGAUGAG GCCGUUAGGC CGAA IGGGAUUG | 8753 |
| 2922 | UGGGAUUC U UCCCCGAU | 1354 | AUCGGGGA CUGAUGAG GCCGUUAGGC CGAA IAAUCCCA | 8754 |
| 2925 | GAUUCUUC C CCGAUCAU | 1355 | AUGAUCGG CUGAUGAG GCCGUUAGGC CGAA IAAGAAUC | 8755 |
| 2926 | AUUCUUCC C CGAUCAUC | 1356 | GAUGAUCG CUGAUGAG GCCGUUAGGC CGAA IGAAGAAU | 8756 |
| 2927 | UUCUUCCC C GAUCAUCA | 1357 | UGAUGAUC CUGAUGAG GCCGUUAGGC CGAA IGGAAGAA | 8757 |
| 2932 | CCCCGAUC A UCAGUUGG | 1358 | CCAACUGA CUGAUGAG GCCGUUAGGC CGAA IAUCGGGG | 8758 |
| 2935 | CGAUCAUC A GUUGGACC | 1359 | GGUCCAAC CUGAUGAG GCCGUUAGGC CGAA IAUGAUCG | 8759 |
| 2943 | AGUUGGAC C CUGCAUUC | 1360 | GAAUGCAG CUGAUCAG GCCGUUAGGC CGAA IUCCAACU | 8760 |
| 2944 | GUUGGACC C UGCAUUCA | 1361 | UGAAUGCA CUGAUGAG GCCGUUAGGC CGAA IGUCCAAC | 8762 |
| 2945 | UUGGACCC U GCAUUCAA | 1362 | UUGAAUGC CUGAUGAG GCCGUUAGGC CGAA IGGUCCAA | 8762 |
| 2948 | GACCCUGC A UUCAAAGC | 1363 | GCUUUGAA CUGAUGAG GCCGUUAGGC CGAA ICAGGGUC | 8763 |
| 2952 | CUGCAUUC A AAGCCAAC | 1364 | GUUGGCUU CUGAUGAG GCCGUUAGGC CGAA IAAUGCAG | 8764 |
| 2957 | UUCAAAGC C AACUCAGU | 1365 | ACUGAGUU CUGAUGAG GCCGUUAGGC CGAA ICUUUGAA | 8765 |
| 2958 | UCAAAGCC A ACUCAGUA | 1366 | UACUGAGU CUGAUGAG GCCGUUAGGC CGAA IGCUUUGA | 8766 |
| 2961 | AAGCCAAC U CAGUAAAU | 1367 | AUUUACUG CUGAUGAG GCCGUUAGGC CGAA IUUGGCUU | 8767 |
| 2963 | GCCAACUC A GUAAAUCC | 1368 | GGAUUUAC CUGAUGAG GCCGUUAGGC CGAA IAGUUGGC | 8768 |
| 2971 | AGUAAAUC C AGAUUGGG | 1369 | CCCAAUCU CUGAUGAG GCCGUUAGGC CGAA IAUUUACU | 8769 |
| 2972 | GUAAAUCC A GAUUGGGA | 1370 | UCCCAAUC CUGAUGAG GCCGUUAGGC CGAA IGAUUUAC | 8770 |
| 2982 | AUUGGGAC C UCAACCCG | 1371 | CGGGUUGA CUGAUGAG GCCGUUAGGC CGAA IUCCCAAU | 8771 |
| 2983 | UUGGGACC U CAACCCGC | 1372 | GCGGGUUG CUGAUGAG GCCGUUAGGC CGAA IGUCCCAA | 8772 |
| 2985 | GGGACCUC A ACCCGCAC | 1373 | GUGCGGGU CUGAUGAG GCCGUUAGGC CGAA IAGGUCCC | 8773 |
| 2988 | ACCUCAAC C CGCACAAG | 1374 | CUUGUGCG CUGAUGAG GCCGUUAGGC CGAA IUUGAGGU | 8774 |
| 2989 | CCUCAACC C GCACAAGG | 1375 | CCUUGUGC CUGAUGAG GCCGUUAGGC CGAA IGUUGAGG | 8775 |
| 2992 | CAACCCGC A CAAGGACA | 1376 | UGUCCUUG CUGAUGAG GCCGUUAGGC CGAA ICGGGUUG | 8776 |
| 2994 | ACCCGCAC A AGGACAAC | 1377 | GUUGUCCU CUGAUGAG GCCGUUAGGC CGAA IUGCGGGU | 8777 |
| 3000 | ACAAGGAC A ACUGGCCG | 1378 | CGGCCAGU CUGAUGAG GCCGUUAGGC CGAA IUCCUUGU | 8778 |
| 3003 | AGGACAAC U GGCCGGAC | 1379 | GUCCGGCC CUGAUGAG GCCGUUAGGC CGAA IUUGUCCU | 8779 |
| 3007 | CAACUGGC C GGACGCCA | 1380 | UGGCGUCC CUGAUGAG GCCGUUAGGC CGAA ICCAGUUG | 8780 |
| 3014 | CCGGACGC C AACAAGGU | 1381 | ACCUUGUU CUGAUGAG GCCGUUAGGC CGAA ICGUCCGG | 8781 |
| 3015 | CGGACGCC A ACAAGGUG | 1382 | CACCUUGU CUGAUGAG GCCGUUAGGC CGAA IGCGUCCG | 8782 |
| 3018 | ACGCCAAC A AGGUGGGA | 1383 | UCCCACCU CUGAUGAG GCCGUUAGGC CGAA IUUGGCGU | 8783 |
| 3035 | GUGGGAGC A UUCGGGCC | 1384 | GGCCCGAA CUGAUGAG GCCGUUAGGC CGAA ICUCCCAC | 8784 |
| 3043 | AUUCGGGC C AGGGUUCA | 1385 | UGAACCCU CUGAUGAG GCCGUUAGGC CGAA ICCCGAAU | 8785 |
| 3044 | UUCGGGCC A GGGUUCAC | 1386 | GUGAACCC CUGAUGAG GCCGUUAGGC CGAA IGCCCGAA | 8786 |
| 3051 | CAGGGUUC A CCCCUCCC | 1387 | GGGAGGGG CUGAUGAG GCCGUUAGGC CGAA IAACCCUG | 8787 |
| 3053 | GGGUUCAC C CCUCCCCA | 1388 | UGGGGAGG CUGAUGAG GCCGUUAGGC CGAA IUGAACCC | 8788 |
| 3054 | GGUUCACC C CUCCCCAU | 1389 | AUGGGGAG CUGAUGAG GCCGUUAGGC CGAA IGUGAACC | 8789 |
| 3055 | GUUCACCC C UCCCCAUG | 1390 | CAUGGGGA CUGAUGAG GCCGUUAGGC CGAA IGGUGAAC | 8790 |
| 3056 | UUCACCCC U CCCCAUGG | 1391 | CCAUGGGG CUGAUGAG GCCGUUAGGC CGAA IGGGUGAA | 8791 |
| 3058 | CACCCCUC C CCAUGGGG | 1392 | CCCCAUGG CUGAUGAG GCCGUUAGGC CGAA IAGGGGUG | 8792 |
| 3059 | ACCCCUCC C CAUGGGGG | 1393 | CCCCCAUG CUGAUGAG GCCGUUAGGC CGAA IGAGGGGU | 8793 |
| 3060 | CCCCUCCC C AUGGGGGA | 1394 | UCCCCCAU CUGAUGAG GCCGUUAGGC CGAA IGGAGGGG | 8794 |
| 3061 | CCCUCCCC A UGGGGGAC | 1395 | GUCCCCCA CUGAUGAG GCCGUUAGGC CGAA IGGGAGGG | 8795 |
| 3070 | UGGGGGAC U GUUGGGGU | 1396 | ACCCCAAC CUGAUGAG GCCGUUAGGC CGAA IUCCCCCA | 8796 |
| 3084 | GGUGGAGC C CUCACGCU | 1397 | AGCGUGAG CUGAUGAG GCCGUUAGGC CGAA ICUCCACC | 8797 |
| 3085 | GUGGAGCC C UCACGCUC | 1398 | GAGCGUGA CUGAUGAG GCCGUUAGGC CGAA IGCUCCAC | 8798 |
| 3086 | UGGAGCCC U CACGCUCA | 1399 | UGAGCGUG CUGAUGAG GCCGUUAGGC CGAA IGGCUCCA | 8799 |
| 3088 | GAGCCCUC A CGCUCAGG | 1400 | CCUGAGCG CUGAUGAG GCCGUUAGGC CGAA IAGGGCUC | 8800 |
| 3092 | CCUCACGC U CAGGGCCU | 1401 | AGGCCCUG CUGAUGAG GCCGUUAGGC CGAA ICGUGAGG | 8801 |
| 3094 | UCACGCUC A GGGCCUAC | 1402 | GUAGGCCC CUGAUGAG GCCGUUAGGC CGAA IAGCGUGA | 8802 |
| 3099 | CUCAGGGC C UACUCACA | 1403 | UGUGAGUA CUGAUGAG GCCGUUAGGC CGAA ICCCUGAG | 8803 |
| 3100 | UCAGGGCC U ACUCACAA | 1404 | UUGUGAGU CUGAUGAG GCCGUUAGGC CGAA IGCCCUGA | 8804 |
| 3103 | GGGCCUAC U CACAACUG | 1405 | CAGUUGUG CUGAUGAG GCCGUUAGGC CGAA IUAGGCCC | 8805 |
| 3105 | GCCUACUC A CAACUGUG | 1406 | CACAGUUG CUGAUGAG GCCGUUAGGC CGAA IAGUAGGC | 8806 |
| 3107 | CUACUCAC A ACUGUGCC | 1407 | GGCACAGU CUGAUGAG GCCGUUAGGC CGAA IUGAGUAG | 8807 |
| 3110 | CUCACAAC U GUGCCAGC | 1408 | GCUGGCAC CUGAUGAG GCCGUUAGGC CGAA IUUGUGAG | 8808 |
| 3115 | AACUGUGC C AGCAGCUC | 1409 | GAGCUGCU CUGAUGAG GCCGUUAGGC CGAA ICACAGUU | 8809 |
| 3116 | ACUGUGCC A GCAGCUCC | 1410 | GGAGCUGC CUGAUGAG GCCGUUAGGC CGAA IGCACAGU | 8810 |
| 3119 | GUGCCAGC A GCUCCUCC | 1411 | GCAGGAGC CUGAUQAG GCCGUUAGGC CGAA ICUGGCAC | 8811 |
| 3122 | CCAGCAGC U CCUCCUCC | 1412 | GGAGGAGG CUGAUGAG GCCGUUAGGC CGAA ICUGCUGG | 8812 |
| 3124 | AGCAGCUC C UCCUCCUG | 1413 | CAGGAGGA CUGAUGAG GCCGUUAGGC CGAA IAGCUGCU | 8813 |
| 3125 | GCAGCUCC U CCUCCUGC | 1414 | GCAGGAGG CUGAUGAG GCCGUUAGGC CGAA IGAGCUGC | 8814 |
| 3127 | AGCUCCUC C UCCUGCCU | 1415 | AGGCAGGA CUGAUGAG GCCGUUAGGC CGAA IAGGAGCU | 8815 |
| 3128 | GCUCCUCC U CCUGCCUC | 1416 | GAGGCAGG CUGAUGAG GCCGUUAGGC CGAA IGAGGAGC | 8816 |
| 3130 | UCCUCCUC C UGCCUCCA | 1417 | UGGAGGCA CUGAUGAG GCCGUUAGGC CGAA IAGGAGGA | 8817 |
| 3131 | CCUCCUCC U GCCUCCAC | 1418 | GUGGAGGC CUGAUGAG GCCGUUAGGC CGAA IGAGGAGG | 8818 |
| 3134 | CCUCCUGC C UCCACCAA | 1419 | UUGGUGGA CUGAUGAG GCCGUUAGGC CGAA ICAGGAGG | 8819 |
| 3135 | CUCCUGCC U CCACCAAU | 1420 | AUUGGUGO CUGAUGAG GCCGUUAGGC CGAA IGCAGGAG | 8820 |
| 3137 | CCUGCCUC C ACCAAUCG | 1421 | CGAUUGGU CUGAUGAG GCCGUUAGGC CGAA IAGGCAGG | 8821 |
| 3138 | CUGCCUCC A CCAAUCGG | 1422 | CCGAUUGG CUGAUGAG GCCGUUAGGC CGAA IGAGGCAG | 8822 |
| 3140 | GCCUCCAC C AAUCGGCA | 1423 | UGCCGAUU CUGAUGAG GCCGUUAGGC CGAA IUGGAGGC | 8823 |
| 3141 | CCUCCACC A AUCGGCAG | 1424 | CUGCCGAU CUGAUGAG GCCGUUAGGC CGAA IGUGGAGG | 8824 |
| 3148 | CAAUCGGC A GUCAGGAA | 1425 | UUCCUGAC CUGAUGAG GCCGUUAGGC CGAA ICCGAUUG | 8825 |
| 3152 | CGGCAGUC A GGAAGGCA | 1426 | UGCCUUCC CUGAUGAG GCCGUUAGGC CGAA IACUGCCG | 8826 |
| 3160 | AGGAAGGC A GCCUACUC | 1427 | GAGUAGGC CUGAUGAG GCCGUUAGGC CGAA ICCUUCCU | 8827 |
| 3163 | AAGGCAGC C UACUCCCU | 1428 | AGGGAGUA CUGAUGAG GCCGUUAGGC CGAA ICUGCCUU | 8828 |
| 3164 | AGGCAGCC U ACUCCCUU | 1429 | AAGGGAGU CUGAUGAG GCCGUUAGGC CGAA IGCUGCCU | 8829 |
| 3167 | CAGCCUAC U CCCUUAUC | 1430 | GAUAAGGG CUGAUGAG GCCGUUAGGC CGAA IUAGGCUG | 8830 |
| 3169 | GCCUACUC C CUUAUCUC | 1431 | GAGAUAAG CUGAUGAG GCCGUUAGGC CGAA IAGUAGGC | 8831 |
| 3170 | CCUACUCC C UUAUCUCC | 1432 | GGAGAUAA CUGAUGAG GCCGUUAGGC CGAA IGAGUAGG | 8832 |
| 3171 | CUACUCCC U UAUCUCCA | 1433 | UGGAGAUA CUGAUGAG GCCGUUAGGC CGAA IGGAGUAG | 8833 |
| 3176 | CCCUUAUC U CCACCUCU | 1434 | AGAGGUGG CUGAUGAG GCCGUUAGGC CGAA IAUAAGGG | 8834 |
| 3178 | CUUAUCUC C ACCUCUAA | 1435 | UUAGAGGU CUGAUGAG GCCGUUAGGC CGAA IAGAUAAG | 8835 |
| 3179 | UUAUCUCC A CCUCUAAG | 1436 | CUUAGAGG CUGAUGAG GCCGUUAGGC CGAA IGAGAUAA | 8836 |
| 3181 | AUCUCCAC C UCUAAGGG | 1437 | CCCUUAGA CUGAUGAG GCCGUUAGGC CGAA IUGGAGAU | 8837 |
| 3182 | UCUCCACC U CUAAGGGA | 1438 | UCCCUUAG CUGAUGAG GCCGUUAGGC CGAA IGUGGAGA | 8838 |
| 3184 | UCCACCUC U AAGGGACA | 1439 | UGUCCCUU CUGAUGAG GCCGUUAGGC CGAA IAGGUGGA | 8839 |
| 3192 | UAAGGGAC A CUCAUCCU | 1440 | AGGAUGAG CUGAUGAG GCCGUUAGGC CGAA IUCCCUUA | 8840 |
| 3194 | AGGGACAC U CAUCCUCA | 1441 | UGAGGAUG CUGAUGAG GCCGUUAGGC CGAA IUGUCCCU | 8841 |
| 3196 | GGACACUC A UCCUCAGG | 1442 | CCUGAGGA CUGAUGAG GCCGUUAGGC CGAA IAGUGUCC | 8842 |
| 3199 | CACUCAUC C UCAGGCCA | 1443 | UGGCCUGA CUGAUGAG GCCGUUAGGC CGAA IAUGAGUG | 8843 |
| 3200 | ACUCAUCC U CAGGCCAU | 1444 | AUGGCCUG CUGAUGAG GCCGUUAGGC CGAA IGAUGAGU | 8844 |
| 3202 | UCAUCCUC A GGCCAUGC | 1445 | GCAUGGCC CUGAUGAG GCCGUUAGGC CGAA IAGGAUGA | 8845 |
| 3206 | CCUCAGGC C AUGCAGUG | 1446 | CACUGCAU CUGAUGAG GCCGUUAGGC CGAA ICCUGAGG | 8846 |
| 3207 | CUCAGGCC A UGCAGUGG | 1447 | CCACUGCA CUGAUGAG GCCGUUAGGC CGAA IGCCUGAG | 8847 |

Input Sequence = AF100308.
Cut Site = CH/.
Stem Length = 8.
Core Sequence = CUGAUGAG X CGAA (X = GCCGUUAGGC or other stem II)
AF100308 (Hepatitis B virus strain 2-18, 3215 bp)
Underlined region can be any X sequence or linker, as described herein.
“I” stands for Inosime

TABLE VII

HUMAN HBV G-CLEAVER AND SUBSTRATE SEQUENCE

| | | | | |
|---|---|---|---|---|
| Pos | Substrate | Seq ID | G-cleaver | Seq ID |

| | | | | |
|---|---|---|---|---|
| 61 | ACUUUCCU G CUGGUGGC | 1448 | GCCACCAG UGAUG GCAUGCACUAUGC GCG AGGAAAGU | 8848 |
| 87 | GGAACAGU G AGCCCUGC | 1449 | GCAGGGCU UGAUG GCAUGCACUAUGC GCG ACUGUUCC | 8849 |
| 94 | UGAGCCCU G CUCAGAAU | 1450 | AUUCUGAG UGAUG GCAUGCACUAUGC GCG AGGGCUCA | 8850 |
| 112 | CUGUCUCU G CCAUAUCG | 1451 | CGAUAUGG UGAUG GCAUGCACUAUGC GCG AGAGACAG | 8851 |
| 132 | AUCUUAUC G AAGACUGG | 1452 | CCAGUCUU UGAUG GCAUGCACUAUGC GCG GAUAAGAU | 8852 |
| 153 | CCUGUACC G AACAUGGA | 1453 | UCCAUGUU UGAUG GCAUGCACUAUGC GCG GGUACAGG | 8853 |
| 169 | AGAACAUC G CAUCAGGA | 1454 | UCCUGAUG UGAUG GCAUGCACUAUGC GCG GAUGUUCU | 8854 |
| 192 | GGACCCCU G CUCGUGUU | 1455 | AACACGAG UGAUG GCAUGCACUAUGC GCG AGGGGUCC | 8855 |
| 222 | UUCUUGUU G ACAAAAAU | 1456 | AUUUUUGU UGAUG GCAUGCACUAUGC GCG AACAAGAA | 8856 |
| 315 | CAAAAUUC G CAGUCCCA | 1457 | UGGGACUG UGAUG GCAUGCACUAUGC GCG GAAUUUUG | 8857 |
| 374 | UGGUUAUC G CUGGAUGU | 1458 | ACAUCCAG UGAUG GCAUGCACUAUGC GCG GAUAACCA | 8858 |
| 387 | AUGUGUCU G CGGCGUUU | 1459 | AAACGCCG UGAUG GCAUGCACUAUGC GCG AGACACAU | 8859 |
| 410 | CUUCCUCU G CAUCCUGC | 1460 | GCAGGAUG UGAUG GCAUGCACUAUGC GCG AGAGGAAG | 8860 |
| 417 | UGCAUCCU G CUGCUAUG | 1461 | CAUAGCAG UGAUG GCAUGCACUAUGC GCG AGGAUGCA | 8861 |
| 420 | AUCCUGCU G CUAUGCCU | 1462 | AGGCAUAG UGAUG GCAUGCACUAUGC GCG AGCAGGAU | 8862 |
| 425 | GCUGCUAU G CCUCAUCU | 1463 | AGAUGAGG UGAUG GCAUGCACUAUGC GCG AUAGCAGC | 8863 |
| 468 | GGUAUGUU G CCCGUUUG | 1464 | CAAACGGG UGAUG GCAUGCACUAUGC GCG AACAUACC | 8864 |
| 518 | CGGACCAU G CAAAACCU | 1465 | AGGUUUUG UGAUG GCAUGCACUAUGC GCG AUGGUCCG | 8865 |
| 527 | CAAAACCU G CACAACUC | 1466 | GAGUUGUG UGAUG GCAUGCACUAUGC GCG AGGUUUUG | 8866 |
| 538 | CAACUCCU G CUCAAGGA | 1467 | UCCUUGAG UGAUG GCAUGCACUAUGC GCG AGGAGUUG | 8867 |
| 569 | CUCAUGUU G CUGUACAA | 1468 | UUGUACAG UGAUG GCAUGCACUAUGC GCG AACAUGAG | 8868 |
| 596 | CGGAAACU G CACCUGUA | 1469 | UACAGGUG UGAUG GCAUGCACUAUGC GCG AGUUUCCG | 8869 |
| 631 | GGGCUUUC G CAAAAUAC | 1470 | GUAUUUUG UGAUG GCAUGCACUAUGC GCG GAAAGCCC | 8870 |
| 687 | UUACUAGU G CCAUUUGU | 1471 | ACAAAUGG UGAUG GCAUGCACUAUGC GCG ACUAGUAA | 8871 |
| 747 | AUAUGGAU G AUGUGGUU | 1472 | AACCACAU UGAUG GCAUGCACUAUGC GCG AUCCAUAU | 8872 |
| 783 | AACAUCUU G AGUCCCUU | 1473 | AAGGGACU UGAUG GCAUGCACUAUGC GCG AAGAUGUU | 8873 |
| 795 | CCCUUUAU G CCGCUGUU | 1474 | AACAGCGG UGAUG GCAUGCACUAUGC GCG AUAAAGGG | 8874 |
| 798 | UUUAUGCC G CUGUUACC | 1475 | GGUAACAG UGAUG GCAUGCACUAUGC GCG GGCAUAAA | 8875 |
| 911 | GGCACAUU G CCACAGGA | 1476 | UCCUGUGG UGAUG GCAUGCACUAUGC GCG AAUGUGCC | 8876 |
| 978 | GGCCUAUU G AUUGGAAA | 1477 | UUUCCAAU UGAUG GCAUGCACUAUGC GCG AAUAGGCC | 8877 |
| 997 | AUGUCAAC G AAUUGUGG | 1478 | CCACAAUU UGAUG GCAUGCACUAUGC GCG GUUGACAU | 8878 |
| 1020 | UGGGGUUU G CCGCCCCU | 1479 | AGGGGCGG UGAUG GCAUGCACUAUGC GCG AAACCCCA | 8879 |
| 1023 | GGUUUGCC G CCCCUUUC | 1480 | GAAAGGGG UGAUG GCAUGCACUAUGC GCG GGCAAACC | 8880 |
| 1034 | CCUUUCAC G CAAUGUGG | 1481 | CCACAUUG UGAUG GCAUGCACUAUGC GCG GUGAAAGG | 8881 |
| 1050 | GAUAUUCU G CUUUAAUG | 1482 | CAUUAAAG UGAUG GCAUGCACUAUGC GCG AGAAUAUC | 8882 |
| 1058 | GCUUUAAU G CCUUUAUA | 1483 | UAUAAAGG UGAUG GCAUGCACUAUGC GCG AUUAAAGC | 8883 |
| 1068 | CUUUAUAU G CAUGCAUA | 1484 | UAUGCAUG UGAUG GCAUGCACUAUGC GCG AUAUAAAG | 8884 |
| 1072 | AUAUGCAU G CAUACAAG | 1485 | CUUGUAUG UGAUG GCAUGCACUAUGC GCG AUGCAUAU | 8885 |
| 1103 | ACUUUCUC G CCAACUUA | 1486 | UAAGUUGG UGAUG GCAUGCACUAUGC GCG GAGAAAGU | 8886 |
| 1139 | CAGUAUGU G AACCUUUA | 1487 | UAAAGGUU UGAUG GCAUGCACUAUGC GCG ACAUACUG | 8887 |
| 1155 | ACCCCGUU G CUCGGCAA | 1488 | UUGCCGAG UGAUG GCAUGCACUAUGC GCG AACGGGGU | 8888 |
| 1177 | UGGUCUAU G CCAAGUGU | 1489 | ACACUUGG UGAUG GCAUGCACUAUGC GCG AUAGACCA | 8889 |
| 1188 | AAGUGUUU G CUGACGCA | 1490 | UGCGUCAG UGAUG GCAUGCACUAUGC GCG AAACACUU | 8890 |
| 1191 | UGUUUGCU G ACGCAACC | 1491 | GGUUGCGU UGAUG GCAUGCACUAUGC GCG AGCAAACA | 8891 |
| 1194 | UUGCUGAC G CAACCCCC | 1492 | GGGGGUUG UGAUG GCAUGCACUAUGC GCG GUCAGCAA | 8892 |
| 1234 | CCAUCAGC G CAUGCGUG | 1493 | CACGCAUG UGAUG GCAUGCACUAUGC GCG GCUGAUGG | 8893 |
| 1238 | CAGCGCAU G CGUGGAAC | 1494 | GUUCCACG UGAUG GCAUGCACUAUGC GCG AUGCGCUG | 8894 |
| 1262 | UCUCCUCU G CCGAUCCA | 1495 | UGGAUCGG UGAUG GCAUGCACUAUGC GCG AGAGGAGA | 8895 |
| 1265 | CCUCUGCC G AUCCAUAC | 1496 | GUAUGGAU UGAUG GCAUGCACUAUGC GCG GGCAGAGG | 8896 |
| 1275 | UCCAUACC G CGGAACUC | 1497 | GAGUUCCG UGAUG GCAUGCACUAUGC GCG GGUAUGGA | 8897 |
| 1290 | UCCUAGCC G CUUGUUUU | 1498 | AAAACAAG UGAUG GCAUGCACUAUGC GCG GGCUAGGA | 8898 |
| 1299 | CUUGUUUU G CUCGCAGC | 1499 | GCUGCGAG UGAUG GCAUGCACUAUGC GCG AAAACAAG | 8899 |
| 1303 | UUUUGCUC G CAGCAGGU | 1500 | ACCUGCUG UGAUG GCAUGCACUAUGC GCG GAGCAAAA | 8900 |
| 1335 | UCGGGACU G ACAAUUCU | 1501 | AGAAUUGU UGAUG GCAUGCACUAUGC GCG AGUCCCGA | 8901 |
| 1349 | UCUGUCGU G CUCUCCCG | 1502 | CGGGAGAG UGAUG GCAUGCACUAUGC GCG ACGACAGA | 8902 |
| 1357 | GCUCUCCC G CAAAUAUA | 1503 | UAUAUUUG UGAUG GCAUGCACUAUGC GCG GGGAGAGC | 8903 |
| 1382 | CCAUGGCU G CUAGGCUG | 1504 | CAGCCUAG UGAUG GCAUGCACUAUGC GCG AGCCAUGG | 8904 |
| 1392 | UAGGCUGU G CUGCCAAC | 1505 | GUUGGCAG UGAUG GCAUGCACUAUGC GCG ACAGCCUA | 8905 |
| 1395 | GCUGUGCU G CCAACUGG | 1506 | CCAGUUGG UGAUG GCAUGCACUAUGC GCG AGCACAGC | 8906 |
| 1411 | GAUCCUAC G CGGGACGU | 1507 | ACGUCCCG UGAUG GCAUGCACUAUGC GCG GUAGGAUC | 8907 |
| 1442 | CCGUCGGC G CUGAAUCC | 1508 | GGAUUCAG UGAUG GCAUGCACUAUGC GCG GCCGACGG | 8908 |
| 1445 | UCGGCGCU G AAUCCCGC | 1509 | GCGGGAUU UGAUG GCAUGCACUAUGC GCG AGCGCCGA | 8909 |
| 1452 | UGAAUCCC G CGGACGAC | 1510 | GUCGUCCG UGAUG GCAUGCACUAUGC GCG GGGAUUCA | 8910 |
| 1458 | CCGCGGAC G ACCCCUCC | 1511 | GGAGGGGU UGAUG GCAUGCACUAUGC GCG GUCCGCGG | 8911 |
| 1474 | CCGGGGCC G CUUGGGGC | 1512 | GCCCCAAG UGAUG GCAUGCACUAUGC GCG GGCCCCGG | 8912 |
| 1489 | GCUCUACC G CCCGCUUC | 1513 | GAAGCGGG UGAUG GCAUGCACUAUGC GCG GGUAGAGC | 8913 |
| 1493 | UACCGCCC G CUUCUCCG | 1514 | CGGAGAAG UGAUG GCAUGCACUAUGC GCG GGGCGGUA | 8914 |
| 1501 | GCUUCUCC G CCUAUUGU | 1515 | ACAAUAGG UGAUG GCAUGCACUAUGC GCG GGAGAAGC | 8915 |
| 1513 | AUUGUACC G ACCGUCCA | 1516 | UGGACGGU UGAUG GCAUGCACUAUGC GCG GGUACAAU | 8916 |
| 1528 | CACGGGGC G CACCUCUC | 1517 | GAGAGGUG UGAUG GCAUGCACUAUGC GCG GCCCCGUG | 8917 |
| 1542 | CUCUUUAC G CGGACUCC | 1518 | GGAGUCCG UGAUG GCAUGCACUAUGC GCG GUAAAGAG | 8918 |
| 1559 | CCGUCUGU G CCUUCUCA | 1519 | UGAGAAGG UGAUG GCAUGCACUAUGC GCG ACAGACGG | 8919 |
| 1571 | UCUCAUCU G CCGGACCG | 1520 | CGGUCCGG UGAUG GCAUGCACUAUGC GCG AGAUGAGA | 8920 |
| 1583 | GACCGUGU G CACUUCGC | 1521 | GCGAAGUG UGAUG GCAUGCACUAUGC GCG ACACGGUC | 8921 |
| 1590 | UGCACUUC G CUUCACCU | 1522 | AGGUGAAG UGAUG GCAUGCACUAUGC GCG GAAGUGCA | 8922 |
| 1601 | UCACCUCU G CACGUCGC | 1523 | GCGACGUG UGAUG GCAUGCACUAUGC GCG AGAGGUGA | 8923 |
| 1608 | UGCACGUC G CAUGGAGA | 1524 | UCUCCAUG UGAUG GCAUGCACUAUGC GCG GACGUGCA | 8924 |
| 1624 | ACCACCGU G AACGCCCA | 1525 | UGGGCGUU UGAUG GCAUGCACUAUGC GCG ACGGUGGU | 8925 |
| 1628 | CCGUGAAC G CCCACAGG | 1526 | CCUGUGGG UGAUG GCAUGCACUAUGC GCG GUUCACGG | 8926 |
| 1642 | AGGAACCU G CCCAAGGU | 1527 | ACCUUGGG UGAUG GCAUGCACUAUGC GCG AGGUUCCU | 8927 |
| 1654 | AAGGUCUU G CAUAAGAG | 1528 | CUCUUAUG UGAUG GCAUGCACUAUGC GCG AAGACCUU | 8928 |
| 1690 | AUGUCAAC G ACCGACCU | 1529 | AGGUCGGU UGAUG GCAUGCACUAUGC GCG GUUGACAU | 8929 |
| 1694 | CAACGACC G ACCUUGAG | 1530 | CUCAAGGU UGAUG GCAUGCACUAUGC GCG GGUCGUUG | 8930 |
| 1700 | CCGACCUU G AGGCAUAC | 1531 | GUAUGCCU UGAUG GCAUGCACUAUGC GCG AAGGUCGG | 8931 |
| 1730 | UGUUUAAU G AGUGGGAG | 1532 | CUCCCACU UGAUG GCAUGCACUAUGC GCG AUUAAACA | 8932 |
| 1818 | AGCACCAU G CAACUUUU | 1533 | AAAAGUUG UGAUG GCAUGCACUAUGC GCG AUGGUGCU | 8933 |
| 1835 | UCACCUCU G CCUAAUCA | 1534 | UGAUUAGG UGAUG GCAUGCACUAUGC GCG AGAGGUGA | 8934 |
| 1883 | CAAGCUGU G CCUUGGGU | 1535 | ACCCAAGG UGAUG GCAUGCACUAUGC GCG ACAGCUUG | 8935 |
| 1912 | UGGACAUU G ACCCGUAU | 1536 | AUACGGGU UGAUG GCAUGCACUAUGC GCG AAUGUCCA | 8936 |
| 1959 | UCUUUUUU G CCUUCUGA | 1537 | UCAGAAGG UGAUG GCAUGCACUAUGC GCG AAAAAAGA | 8937 |
| 1966 | UGCCUUCU G ACUUCUUU | 1538 | AAAGAAGU UGAUG GCAUGCACUAUGC GCG AGAAGGCA | 8938 |
| 1985 | UUCUAUUC G AGAUCUCC | 1539 | GGAGAUCU UGAUG GCAUGCACUAUGC GCG GAAUAGAA | 8939 |
| 1996 | AUCUCCUC G ACACCGCC | 1540 | GGCGGUGU UGAUG GCAUGCACUAUGC GCG GAGGAGAU | 8940 |
| 2002 | UCGACACC G CCUCUGCU | 1541 | AGCAGAGG UGAUG GCAUGCACUAUGC GCG GGUGUCGA | 8941 |
| 2008 | CCGCCUCU G CUCUGUAU | 1542 | AUACAGAG UGAUG GCAUGCACUAUGC GCG AGAGGCGG | 8942 |
| 2092 | GUUGGGGU G AGUUGAUG | 1543 | CAUCAACU UGAUG GCAUGCACUAUGC GCG ACCCCAAC | 8943 |
| 2097 | GGUGAGUU G AUGAAUCU | 1544 | AGAUUCAU UGAUG GCAUGCACUAUGC GCG AACUCACC | 8944 |
| 2100 | GAGUUGAU G AAUCUAGC | 1545 | GCUAGAUU UGAUG GCAUGCACUAUGC GCG AUCAACUC | 8945 |
| 2237 | UUUUGGGC G AGAAACUG | 1546 | CAGUUUCU UGAUG GCAUGCACUAUGC GCG GCCCAAAA | 8946 |
| 2251 | CUGUUCUU G AAUAUUUG | 1547 | CAAAUAUU UGAUG GCAUGCACUAUGC GCG AAGAACAG | 8947 |
| 2282 | GUGGAUUC G CACUCCUC | 1548 | GAGGAGUG UGAUG GCAUGCACUAUGC GCG GAAUCCAC | 8948 |
| 2293 | CUCCUCCU G CAUAUAGA | 1549 | UCUAUAUG UGAUG GCAUGCACUAUGC GCG AGGAGGAG | 8949 |
| 2311 | CACCAAAU G CCCCUAUC | 1550 | GAUAGGGG UGAUG GCAUGCACUAUGC GCG AUUUGGUG | 8950 |
| 2354 | UGUUAGAC G AAGAGGCA | 1551 | UGCCUCUU UGAUG GCAUGCACUAUGC GCG GUCUAACA | 8951 |
| 2388 | ACUCCCUC G CCUCGCAG | 1552 | CUGCGAGG UGAUG GCAUGCACUAUGC GCG GAGGGAGU | 8952 |
| 2393 | CUCGCCUC G CAGACGAA | 1553 | UUCGUCUG UGAUG GCAUGCACUAUGC GCG GAGGCGAG | 8953 |
| 2399 | UCGCAGAC G AAGGUCUC | 1554 | GAGACCUU UGAUG GCAUGCACUAUGC GCG GUCUGCGA | 8954 |
| 2412 | UCUCAAUC G CCGCGUCG | 1555 | CGACGCGG UGAUG GCAUGCACUAUGC GCG GAUUGAGA | 8955 |
| 2415 | CAAUCGCC G CGUCGCAG | 1556 | CUGCGACG UGAUG GCAUGCACUAUGC GCG GGCGAUUG | 8956 |
| 2420 | GCCGCGUC G CAGAAGAU | 1557 | AUCUUCUG UGAUG GCAUGCACUAUGC GCG GACGCGGC | 8957 |
| 2514 | GGUACCUU G CUUUAAUC | 1558 | GAUUAAAG UGAUG GCAUGCACUAUGC GCG AAGGUACC | 8958 |
| 2549 | CUUUUCCU G ACAUUCAU | 1559 | AUGAAUGU UGAUG GCAUGCACUAUGC GCG AGGAAAAG | 8959 |
| 2560 | AUUCAUUU G CAGGAGGA | 1560 | UCCUCCUG UGAUG GCAUGCACUAUGC GCG AAAUGAAU | 8960 |
| 2576 | ACAUUGUU G AUAGAUGU | 1561 | ACAUCUAU UGAUG GCAUGCACUAUGC GCG AACAAUGU | 8961 |
| 2615 | CAGUAAAU G AAAACAGG | 1562 | CCUGUUUU UGAUG GCAUGCACUAUGC GCG AUUUACUG | 8962 |
| 2641 | UUAACUAU G CCUGCUAG | 1563 | CUAGCAGG UGAUG GCAUGCACUAUGC GCG AUAGUUAA | 8963 |
| 2645 | CUAUGCCU G CUAGGUUU | 1564 | AAACCUAG UGAUG GCAUGCACUAUGC GCG AGGCAUAG | 8964 |
| 2677 | AAAUAUUU G CCCUUAGA | 1565 | UCUAAGGG UGAUG GCAUGCACUAUGC GCG AAAUAUUU | 8965 |
| 2740 | UUCCAGAC G CGACAUUA | 1566 | UAAUGUCG UGAUG GCAUGCACUAUGC GCG GUCUGGAA | 8966 |
| 2742 | CCAGACGC G ACAUUAUU | 1567 | AAUAAUGU UGAUG GCAUGCACUAUGC GCG GCGUCUGG | 8967 |
| 2804 | CACGUAGC G CCUCAUUU | 1568 | AAAUGAGG UGAUG GCAUGCACUAUGC GCG GCUACGUG | 8968 |
| 2814 | CUCAUUUU G CGGGUCAC | 1569 | GUGACCCG UGAUG GCAUGCACUAUGC GCG AAAAUGAG | 8969 |
| 2875 | CAAACCUC G AAAAGGCA | 1570 | UGCCUUUU UGAUG GCAUGCACUAUGC GCG GAGGUUUG | 8970 |
| 2928 | UCUUCCCC G AUCAUCAG | 1571 | CUGAUGAU UGAUG GCAUGCACUAUGC GCG GGGGAAGA | 8971 |
| 2946 | UGGACCCU G CAUUCAAA | 1572 | UUUGAAUG UGAUG GCAUGCACUAUGC GCG AGGGUCCA | 8972 |
| 2990 | CUCAACCC G CACAAGGA | 1573 | UCCUUGUG UGAUG GCAUGCACUAUGC GCG GGGUUGAG | 8973 |
| 3012 | GGCCGGAC G CCAACAAG | 1574 | CUUGUUGG UGAUG GCAUGCACUAUGC GCG GUCCGGCC | 8974 |
| 3090 | GCCCUCAC G CUCAGGGC | 1575 | GCCCUGAG UGAUG GCAUGCACUAUGC GCG GUGAGGGC | 8975 |
| 3113 | ACAACUGU G CCAGCAGC | 1576 | GCUGCUGG UGAUG GCAUGCACUAUGC GCG ACAGUUGU | 8976 |
| 3132 | CUCCUCCU G CCUCCACC | 1577 | GGUGGAGG UGAUG GCAUGCACUAUGC GCG AGGAGGAG | 8977 |
| 51 | AGGGCCCU G UACUUUCC | 1578 | GGAAAGUA UGAUG GCAUGCACUAUGC GCG AGGGCCCU | 8978 |
| 106 | AGAAUACU G UCUCUGCC | 1579 | GGCAGAGA UGAUG GCAUGCACUAUGC GCG AGUAUUCU | 8979 |
| 148 | GGGACCCU G UACCGAAC | 1580 | GUUCGGUA UGAUG GCAUGCACUAUGC GCG AGGGUCCC | 8980 |
| 198 | CUGCUCGU G UUACAGGC | 1581 | GCCUGUAA UGAUG GCAUGCACUAUGC GCG ACGAGCAG | 8981 |
| 219 | UUUUUCUU G UUGACAAA | 1582 | UUUGUCAA UGAUG GCAUGCACUAUGC GCG AAGAAAAA | 8982 |
| 297 | ACACCCGU G UGUCUUGG | 1583 | CCAAGACA UGAUG GCAUGCACUAUGC GCG ACGGGUGU | 8983 |
| 299 | ACCCGUGU G UCUUGGCC | 1584 | GGCCAAGA UGAUG GCAUGCACUAUGC GCG ACACGGGU | 8984 |
| 347 | ACCAACCU G UUGUCCUC | 1585 | GAGGACAA UGAUG GCAUGCACUAUGC GCG AGGUUGGU | 8985 |
| 350 | AACCUGUU G UCCUCCAA | 1586 | UUGGAGGA UGAUG GCAUGCACUAUGC GCG AACAGGUU | 8986 |
| 362 | UCCAAUUU G UCCUGGUU | 1587 | AACCAGGA UGAUG GCAUGCACUAUGC GCG AAAUUGGA | 8987 |
| 381 | CGCUGGAU G UGUCUGCG | 1588 | CGCAGACA UGAUG GCAUGCACUAUGC GCG AUCCAGCG | 8988 |
| 383 | CUGGAUGU G UCUGCGGC | 1589 | GCCGCAGA UGAUG GCAUGCACUAUGC GCG ACAUCCAG | 8989 |
| 438 | AUCUUCUU G UUGGUUCU | 1590 | AGAACCAA UGAUG GCAUGCACUAUGC GCG AAGAAGAU | 8990 |
| 465 | CAAGGUAU G UUGCCCGU | 1591 | ACGGGCAA UGAUG GCAUGCACUAUGC GCG AUACCUUG | 8991 |
| 476 | GCCCGUUU G UCCUCUAA | 1592 | UUAGAGGA UGAUG GCAUGCACUAUGC GCG AAACGGGC | 8992 |
| 555 | ACCUCUAU G UUUCCCUC | 1593 | GAGGGAAA UGAUG GCAUGCACUAUGC GCG AUAGAGGU | 8993 |
| 566 | UCCCUCAU G UUGCUGUA | 1594 | UACAGCAA UGAUG GCAUGCACUAUGC GCG AUGAGGGA | 8994 |
| 572 | AUGUUGCU G UACAAAAC | 1595 | GUUUUGUA UGAUG GCAUGCACUAUGC GCG AGCAACAU | 8995 |
| 602 | CUGCACCU G UAUUCCCA | 1596 | UGGGAAUA UGAUG GCAUGCACUAUGC GCG AGGUGCAG | 8996 |
| 694 | UGCCAUUU G UUCAGUGG | 1597 | CCACUGAA UGAUG GCAUGCACUAUGC GCG AAAUGGCA | 8997 |
| 724 | CCCCCACU G UCUGGCUU | 1598 | AAGCCAGA UGAUG GCAUGCACUAUGC GCG AGUGGGGG | 8998 |
| 750 | UGGAUGAU G UGGUUUUG | 1599 | CAAAACCA UGAUG GCAUGCACUAUGC GCG AUCAUCCA | 8999 |
| 771 | CCAAGUCU G UACAACAU | 1600 | AUGUUGUA UGAUG GCAUGCACUAUGC GCG AGACUUGG | 9000 |
| 801 | AUGCCGCU G UUACCAAU | 1601 | AUUGGUAA UGAUG GCAUGCACUAUGC GCG AGCGGCAU | 9001 |
| 818 | UUUCUUUU G UCUUUGGG | 1602 | CCCAAAGA UGAUG GCAUGCACUAUGC GCG AAAAGAAA | 9002 |
| 888 | UGGGAUAU G UAAUUGGG | 1603 | CCCAAUUA UGAUG GCAUGCACUAUGC GCG AUAUCCCA | 9003 |
| 927 | AACAUAUU G UACAAAAA | 1604 | UUUUUGUA UGAUG GCAUGCACUAUGC GCG AAUAUGUU | 9004 |
| 944 | AUCAAAAU G UGUUUUAG | 1605 | CUAAAACA UGAUG GCAUGCACUAUGC GCG AUUUUGAU | 9005 |
| 946 | CAAAAUGU G UUUUAGGA | 1606 | UCCUAAAA UGAUG GCAUGCACUAUGC GCG ACAUUUUG | 9006 |
| 963 | AACUUCCU G UAAACAGG | 1607 | CCUGUUUA UGAUG GCAUGCACUAUGC GCG AGGAAGUU | 9007 |
| 991 | GAAAGUAU G UCAACGAA | 1608 | UUCGUUGA UGAUG GCAUGCACUAUGC GCG AUACUUUC | 9008 |
| 1002 | AACGAAUU G UGGGUCUU | 1609 | AAGACCCA UGAUG GCAUGCACUAUGC GCG AAUUCGUU | 9009 |
| 1039 | CACGCAAU G UGGAUAUU | 1610 | AAUAUCCA UGAUG GCAUGCACUAUGC GCG AUUGCGUG | 9010 |
| 1137 | AACAGUAU G UGAACCUU | 1611 | AAGGUUCA UGAUG GCAUGCACUAUGC GCG AUACUGUU | 9011 |
| 1184 | UGCCAAGU G UUUGCUGA | 1612 | UCAGCAAA UGAUG GCAUGCACUAUGC GCG ACUUGGCA | 9012 |
| 1251 | GAACCUUU G UGUCUCCU | 1613 | AGGAGACA UGAUG GCAUGCACUAUGC GCG AAAGGUUC | 9013 |
| 1253 | ACCUUUGU G UCUCCUCU | 1614 | AGAGGAGA UGAUG GCAUGCACUAUGC GCG ACAAAGGU | 9014 |
| 1294 | AGCCGCUU G UUUUGCUC | 1615 | GAGCAAAA UGAUG GCAUGCACUAUGC GCG AAGCGCCU | 9015 |
| 1344 | ACAAUUCU G UCGUGCUC | 1616 | GAGCACGA UGAUG GCAUGCACUAUGC GCG AGAAUUGU | 9016 |
| 1390 | GCUAGGCU G UGCUGCCA | 1617 | UGGCAGCA UGAUG GCAUGCACUAUGC GCG AGCCUAGC | 9017 |
| 1425 | CGUCCUUU G UUUACGUC | 1618 | GACGUAAA UGAUG GCAUGCACUAUGC GCG AAAGGACG | 9018 |
| 1508 | CGCCUAUU G UACCGACC | 1619 | GGUCGGUA UGAUG GCAUGCACUAUGC GCG AAUAGGCG | 9019 |
| 1557 | CCCCGUCU G UGCCUUCU | 1620 | AGAAGGCA UGAUG GCAUGCACUAUGC GCG AGACGGGG | 9020 |
| 1581 | CGGACCGU G UGCACUUC | 1621 | GAAGUGCA UGAUG GCAUGCACUAUGC GCG ACGGUCCG | 9021 |
| 1684 | UCAGCAAU G UCAACGAC | 1622 | GUCGUUGA UGAUG GCAUGCACUAUGC GCG AUUGCUGA | 9022 |
| 1719 | CAAAGACU G UGUGUUUA | 1623 | UAAACACA UGAUG GCAUGCACUAUGC GCG AGUCUUUG | 9023 |
| 1721 | AAGACUGU G UGUUUAAU | 1624 | AUUAAACA UGAUG GCAUGCACUAUGC GCG ACAGUCUU | 9024 |
| 1723 | GACUGUGU G UUUAAUGA | 1625 | UCAUUAAA UGAUG GCAUGCACUAUGC GCG ACACAGUC | 9025 |
| 1772 | AGGUCUUU G UACUAGGA | 1626 | UCCUAGUA UGAUG GCAUGCACUAUGC GCG AAAGACCU | 9026 |
| 1785 | AGGAGGCU G UAGGCAUA | 1627 | UAUGCCUA UGAUG GCAUGCACUAUGC GCG AGCCUCCU | 9027 |
| 1801 | AAAUUGGU G UGUUCACC | 1628 | GGUGAACA UGAUG GCAUGCACUAUGC GCG ACCAAUUU | 9028 |
| 1803 | AUUGGUGU G UUCACCAG | 1629 | CUGGUGAA UGAUG GCAUGCACUAUGC GCG ACACCAAU | 9029 |
| 1850 | CAUCUCAU G UUCAUGUC | 1630 | GACAUGAA UGAUG GCAUGCACUAUGC GCG AUGAGAUG | 9030 |
| 1856 | AUGUUCAU G UCCUACUG | 1631 | CAGUAGGA UGAUG GCAUGCACUAUGC GCG AUGAACAU | 9031 |
| 1864 | GUCCUACU G UUCAAGCC | 1632 | GGCUUGAA UGAUG GCAUGCACUAUGC GCG AGUAGGAC | 9032 |
| 1881 | UCCAAGCU G UGCCUUGG | 1633 | CCAAGGCA UGAUG GCAUGCACUAUGC GCG AGCUUGGA | 9033 |
| 1939 | GAGCUUCU G UGGAGUUA | 1634 | UAACUCCA UGAUG GCAUGCACUAUGC GCG AGAAGCUC | 9034 |
| 2013 | UCUGCUCU G UAUCGGGG | 1635 | CCCCGAUA UGAUG GCAUGCACUAUGC GCG AGAGCAGA | 9035 |
| 2045 | GGAACAUU G UUCACCUC | 1636 | GAGGUGAA UGAUG GCAUGCACUAUGC GCG AAUGUUCC | 9036 |
| 2082 | GCUAUUCU G UGUUGGGG | 1637 | CCCCAACA UGAUG GCAUGCACUAUGC GCG AGAAUAGC | 9037 |
| 2084 | UAUUCUGU G UUGGGGUG | 1638 | CACCCCAA UGAUG GCAUGCACUAUGC GCG ACAGAAUA | 9038 |
| 2167 | UCAGCUAU G UCAACGUU | 1639 | AACGUUGA UGAUG GCAUGCACUAUGC GCG AUAGCUGA | 9039 |
| 2205 | CAACUAUU G UGGUUUCA | 1640 | UGAAACCA UGAUG GCAUGCACUAUGC GCG AAUAGUUG | 9040 |
| 2222 | CAUUUCCU G UCUUACUU | 1641 | AAGUAAGA UGAUG GCAUGCACUAUGC GCG AGGAAAUG | 9041 |
| 2245 | GAGAAACU G UUCUUGAA | 1642 | UUCAAGAA UGAUG GCAUGCACUAUGC GCG AGUUUCUC | 9042 |
| 2262 | UAUUUGGU G UCUUUUGG | 1643 | CCAAAAGA UGAUG GCAUGCACUAUGC GCG ACCAAAUA | 9043 |
| 2274 | UUUGGAGU G UGGAUUCG | 1644 | CGAAUCCA UGAUG GCAUGCACUAUGC GCG ACUCCAAA | 9044 |
| 2344 | AAACUACU G UUGUUAGA | 1645 | UCUAACAA UGAUG GCAUGCACUAUGC GCG AGUAGUUU | 9045 |
| 2347 | CUACUGUU G UUAGACGA | 1646 | UCGUCUAA UGAUG GCAUGCACUAUGC GCG AACAGUAG | 9046 |
| 2450 | AUCUCAAU G UUAGUAUU | 1647 | AAUACUAA UGAUG GCAUGCACUAUGC GCG AUUGAGAU | 9047 |
| 2573 | AGGACAUU G UUGAUAGA | 1648 | UCUAUCAA UGAUG GCAUGCACUAUGC GCG AAUGUCCU | 9048 |
| 2583 | UGAUAGAU G UAAGCAAU | 1649 | AUUGCUUA UGAUG GCAUGCACUAUGC GCG AUCUAUCA | 9049 |
| 2594 | AGCAAUUU G UGGGGCCC | 1650 | GGGCCCCA UGAUG GCAUGCACUAUGC GCG AAAUUGCU | 9050 |
| 2663 | AUCCCAAU G UUACUAAA | 1651 | UUUAGUAA UGAUG GCAUGCACUAUGC GCG AUUGGGAU | 9051 |
| 2717 | CAGAGUAU G UAGUUAAU | 1652 | AUUAACUA UGAUG GCAUGCACUAUGC GCG AUACUCUG | 9052 |
| 2901 | AUCUUUCU G UCCCCAAU | 1653 | AUUGGGGA UGAUG GCAUGCACUAUGC GCG AGAAAGAU | 9053 |
| 3071 | GGGGGACU G UUGGGGUG | 1654 | CACCCCAA UGAUG GCAUGCACUAUGC GCG AGUCCCCC | 9054 |
| 3111 | UCACAACU G UGCCAGCA | 1655 | UGCUGGCA UGAUG GCAUGCACUAUGC GCG AGUUGUGA | 9055 |

Input Sequence = AF100308.
Cut Site = YG/M or UG/U.
Stem Length = 8.
Core Sequence = UGAUG GCAUGCACUAUGC GCG
AF100308 (Hepatitis B virus strain 2-18, 3215 bp)

TABLE VIII

HUMAN HBV ZINZYME AND SUBSTRATE SEQUENCE

| | | | | |
|---|---|---|---|---|
| Pos | Substrate | Seq ID | Zinzyme | Seq ID |

| | | | | |
|---|---|---|---|---|
| 61 | ACUUUCCU G CUGGUGGC | 1448 | GCCACCAG GCcgaaagGCGaGuCaaGGuCu AGGAAAGU | 9056 |
| 94 | UGAGCCCU G CUCAGAAU | 1450 | AUUCUGAG GCcgaaagGCGaGuCaaGGuCu AGGGCUCA | 9057 |
| 112 | CUGUCUCU G CCAUAUCG | 1451 | CGAUAUGG GCcgaaagGCGaGuCaaGGuCu AGAGACAG | 9058 |
| 169 | AGAACAUC G CAUCAGGA | 1454 | UCCUGAUG GCcgaaagGCGaGuCaaGGuCu GAUGUUCU | 9059 |
| 192 | GGACCCCU G CUCGUGUU | 1455 | AACACGAG GCcgaaagGCGaGuCaaGGuCu AGGGGUCC | 9060 |
| 315 | CAAAAUUC G CAGUCCCA | 1457 | UGGGACUG GCcgaaagGCGaGuCaaGGuCu GAAUUUUG | 9061 |
| 374 | UGGUUAUC G CUGGAUGU | 1458 | ACAUCCAG GCcgaaagGCGaGuCaaGGuCu GAUAACCA | 9062 |
| 387 | AUGUGUCU G CGGCGUUU | 1459 | AAACGCCG GCcgaaagGCGaGuCaaGGuCu AGACACAU | 9063 |
| 410 | CUUCCUCU G CAUCCUGC | 1460 | GCAGGAUG GCcgaaagGCGaGuCaaGGuCu AGAGGAAG | 9064 |
| 417 | UGCAUCCU G CUGCUAUG | 1461 | CAUAGCAG GCcgaaagGCGaGuCaaGGuCu AGGAUGCA | 9065 |
| 420 | AUCCUGCU G CUAUGCCU | 1462 | AGGCAUAG GCcgaaagGCGaGuCaaGGuCu AGCAGGAU | 9066 |
| 425 | GCUGCUAU G CCUCAUCU | 1463 | AGAUGAGG GCcgaaagGCGaGuCaaGGuCu AUAGCAGC | 9067 |
| 468 | GGUAUGUU G CCCGUUUG | 1464 | CAAACGGG GCcgaaagGCGaGuCaaGGuCu AACAUACC | 9068 |
| 518 | CGGACCAU G CAAAACCU | 1465 | AGGUUUUG GCcgaaagGCGaGuCaaGGuCu AUGGUCCG | 9069 |
| 527 | CAAAACCU G CACAACUC | 1466 | GAGUUGUG GCcgaaagGCGaGuCaaGGuCu AGGUUUUG | 9070 |
| 538 | CAACUCCU G CUCAAGGA | 1467 | UCCUUGAG GCcgaaagGCGaGuCaaGGuCu AGGAGUUG | 9071 |
| 569 | CUCAUGUU G CUGUACAA | 1468 | UUGUACAG GCcgaaagGCGaGuCaaGGuCu AACAUGAG | 9072 |
| 596 | CGGAAACU G CACCUGUA | 1469 | UACAGGUG GCcgaaagGCGaGuCaaGGuCu AGUUUCCG | 9073 |
| 631 | GGGCUUUC G CAAAAUAC | 1470 | GUAUUUUG GCcgaaagGCGaGuCaaGGuCu GAAAGCCC | 9074 |
| 687 | UUACUAGU G CCAUUUGU | 1471 | ACAAAUGG GCcgaaagGCGaGuCaaGGuCu ACUAGUAA | 9075 |
| 795 | CCCUUUAU G CCGCUGUU | 1474 | AACAGCGG GCcgaaagGCGaGuCaaGGuCu AUAAAGGG | 9076 |
| 798 | UUUAUGCC G CUGUUACC | 1475 | GGUAACAG GCcgaaagGCGaGuCaaGGuCu GGCAUAAA | 9077 |
| 911 | GGCACAUU G CCACAGGA | 1476 | UCCUGUGG GCcgaaagGCGaGuCaaGGuCu AAUGUGCC | 9078 |
| 1020 | UGGGGUUU G CCGCCCCU | 1479 | AGGGGCGG GCcgaaagGCGaGuCaaGGuCu AAACCCCA | 9079 |
| 1023 | GGUUUGCC G CCCCUUUC | 1480 | GAAAGGGG GCcgaaagGCGaGuCaaGGuCu GGCAAACC | 9080 |
| 1034 | CCUUUCAC G CAAUGUGG | 1481 | CCACAUUG GCcgaaagGCGaGuCaaGGuCu GUGAAAGG | 9081 |
| 1050 | GAUAUUCU G CUUUAAUG | 1482 | CAUUAAAG GCcgaaagGCGaGuCaaGGuCu AGAAUAUC | 9082 |
| 1058 | GCUUUAAU G CCUUUAUA | 1483 | UAUAAAGG GCcgaaagGCGaGuCaaGGuCu AUUAAAGC | 9083 |
| 1068 | CUUUAUAU G CAUGCAUA | 1484 | UAUGCAUG GCcgaaagGCGaGuCaaGGuCu AUAUAAAG | 9084 |
| 1072 | AUAUGCAU G CAUACAAG | 1485 | CUUGUAUG GCcgaaagGCGaGuCaaGGuCu AUGCAUAU | 9085 |
| 1103 | ACUUUCUC G CCAACUUA | 1486 | UAAGUUGG GCcgaaagGCGaGuCaaGGuCu GAGAAAGU | 9086 |
| 1155 | ACCCCGUU G CUCGGCAA | 1488 | UUGCCGAG GCcgaaagGCGaGuCaaGGuCu AACGGGGU | 9087 |
| 1177 | UGGUCUAU G CCAAGUGU | 1489 | ACACUUGG GCcgaaagGCGaGuCaaGGuCu AUAGACCA | 9088 |
| 1188 | AAGUGUUU G CUGACGCA | 1490 | UGCGUCAG GCcgaaagGCGaGuCaaGGuCu AAACACUU | 9089 |
| 1194 | UUGCUGAC G CAACCCCC | 1492 | GGGGGUUG GCcgaaagGCGaGuCaaGGuCu GUCAGCAA | 9090 |
| 1234 | CCAUCAGC G CAUGCGUG | 1493 | CACGCAUG GCcgaaagGCGaGuCaaGGuCu GCUGAUGG | 9091 |
| 1238 | CAGCGCAU G CGUGGAAC | 1494 | GUUCCACG GCcgaaagGCGaGuCaaGGuCu AUGCGCUG | 9092 |
| 1262 | UCUCCUCU G CCGAUCCA | 1495 | UGGAUCGG GCcgaaagGCGaGuCaaGGuCu AGAGGAGA | 9093 |
| 1275 | UCCAUACC G CGGAACUC | 1497 | GAGUUCCG GCcgaaagGCGaGuCaaGGuCu GGUAUGGA | 9094 |
| 1290 | UCCUAGCC G CUUGUUUU | 1498 | AAAACAAG GCcgaaagGCGaGuCaaGGuCu GGCUAGGA | 9095 |
| 1299 | CUUGUUUU G CUCGCAGC | 1499 | GCUGCGAG GCcgaaagGCGaGuCaaGGuCu AAAACAAG | 9096 |
| 1303 | UUUUGCUC G CAGCAGGU | 1500 | ACCUGCUG GCcgaaagGCGaGuCaaGGuCu GAGCAAAA | 9097 |
| 1349 | UCUGUCGU G CUCUCCCG | 1502 | CGGGAGAG GCcgaaagGCGaGuCaaGGuCu ACGACAGA | 9098 |
| 1357 | GCUCUCCC G CAAAUAUA | 1503 | UAUAUUUG GCcgaaagGCGaGuCaaGGuCu GGGAGAGC | 9099 |
| 1382 | CCAUGGCU G CUAGGCUG | 1504 | CAGCCUAG GCcgaaagGCGaGuCaaGGuCu AGCCAUGG | 9100 |
| 1392 | UAGGCUGU G CUGCCAAC | 1505 | GUUGGCAG GCcgaaagGCGaGuCaaGGuCu ACAGCCUA | 9101 |
| 1395 | GCUGUGCU G CCAACUGG | 1506 | CCAGUUGG GCcgaaagGCGaGuCaaGGuCu AGCACAGC | 9102 |
| 1411 | GAUCCUAC G CGGGACGU | 1507 | ACGUCCCG GCcgaaagGCGaGuCaaGGuCu GUAGGAUC | 9103 |
| 1442 | CCGUCGGC G CUGAAUCC | 1508 | GGAUUCAG GCcgaaagGCGaGuCaaGGuCu GCCGACGG | 9104 |
| 1452 | UGAAUCCC G CGGACGAC | 1510 | GUCGUCCG GCcgaaagGCGaGuCaaGGuCu GGGAUUCA | 9105 |
| 1474 | CCGGGGCC G CUUGGGGC | 1512 | GCCCCAAG GCcgaaagGCGaGuCaaGGuCu GGCCCCGG | 9106 |
| 1489 | GCUCUACC G CCCGCUUC | 1513 | GAAGCGGG GCcgaaagGCGaGuCaaGGuCu GGUAGAGC | 9107 |
| 1493 | UACCGCCC G CUUCUCCG | 1514 | CGGAGAAG GCcgaaagGCGaGuCaaGGuCu GGGCGGUA | 9108 |
| 1501 | GCUUCUCC G CCUAUUGU | 1515 | ACAAUAGG GCcgaaagGCGaGuCaaGGuCu GGAGAAGC | 9109 |
| 1528 | CACGGGGC G CACCUCUC | 1517 | GAGAGGUG GCcgaaagGCGaGuCaaGGuCu GCCCCGUG | 9110 |
| 1542 | CUCUUUAC G CGGACUCC | 1518 | GGAGUCCG GCcgaaagGCGaGuCaaGGuCu GUAAAGAG | 9111 |
| 1559 | CCGUCUGU G CCUUCUCA | 1519 | UGAGAAGG GCcgaaagGCGaGuCaaGGuCu ACAGACGG | 9112 |
| 1571 | UCUCAUCU G CCGGACCG | 1520 | CGGUCCGG GCcgaaagGCGaGuCaaGGuCu AGAUGAGA | 9113 |
| 1583 | GACCGUGU G CACUUCGC | 1521 | GCGAAGUG GCcgaaagGCGaGuCaaGGuCu ACACGGUC | 9114 |
| 1590 | UGCACUUC G CUUCACCU | 1522 | AGGUGAAG GCcgaaagGCGaGuCaaGGuCu GAAGUGCA | 9115 |
| 1601 | UCACCUCU G CACGUCGC | 1523 | GCGACGUG GCcgaaagGCGaGuCaaGGuCu AGAGGUGA | 9116 |
| 1608 | UGCACGUC G CAUGGAGA | 1524 | UCUCCAUG GCcgaaagGCGaGuCaaGGuCu GACGUGCA | 9117 |
| 1628 | CCGUGAAC G CCCACAGG | 1526 | CCUGUGGG GCcgaaagGCGaGuCaaGGuCu GUUCACGG | 9118 |
| 1642 | AGGAACCU G CCCAAGGU | 1527 | ACCUUGGG GCcgaaagGCGaGuCaaGGuCu AGGUUCCU | 9119 |
| 1654 | AAGGUCUU G CAUAAGAG | 1528 | CUCUUAUG GCcgaaagGCGaGuCaaGGuCu AAGACCUU | 9120 |
| 1818 | AGCACCAU G CAACUUUU | 1533 | AAAAGUUG GCcgaaagGCGaGuCaaGGuCu AUGGUGCU | 9121 |
| 1835 | UCACCUCU G CCUAAUCA | 1534 | UGAUUAGG GCcgaaagGCGaGuCaaGGuCu AGAGGUGA | 9122 |
| 1883 | CAAGCUGU G CCUUGGGU | 1535 | ACCCAAGG GCcgaaagGCGaGuCaaGGuCu ACAGCUUG | 9123 |
| 1959 | UCUUUUUU G CCUUCUGA | 1537 | UCAGAAGG GCcgaaagGCGaGuCaaGGuCu AAAAAACA | 9124 |
| 2002 | UCGACACC G CCUCUGCU | 1541 | AGCAGAGG GCcgaaagGCGaGuCaaGGuCu GGUGUCGA | 9125 |
| 2008 | CCGCCUCU G CUCUGUAU | 1542 | AUACAGAG GCcgaaagGCGaGuCaaGGuCu AGAGGCGG | 9126 |
| 2282 | GUGGAUUC G CACUCCUC | 1548 | GAGGAGUG GCcgaaagGCGaGuCaaGGuCu GAAUCCAC | 9127 |
| 2293 | CUCCUCCU G CAUAUAGA | 1549 | UCUAUAUG GCcgaaagGCGaGuCaaGGuCu AGGAGGAG | 9128 |
| 2311 | CACCAAAU G CCCCUAUC | 1550 | GAUAGGGG GCcgaaagGCGaGuCaaGGuCu AUUUGGUG | 9129 |
| 2388 | ACUCCCUC G CCUCGCAG | 1552 | CUGCGAGG GCcgaaagGCGaGuCaaGGuCu GAGGGAGU | 9130 |
| 2393 | CUCGCCUC G CAGACGAA | 1553 | UUCGUCUG GCcgaaagGCGaGuCaaGGuCu GAGGCGAG | 9131 |
| 2412 | UCUCAAUC G CCGCGUCG | 1555 | CGACGCGG GCcgaaagGCGaGuCaaGGuCu GAUUGAGA | 9132 |
| 2415 | CAAUCGCC G CGUCGCAG | 1556 | CUGCGACG GCcgaaagGCGaGuCaaGGuCu GGCGAUUG | 9133 |
| 2420 | GCCGCGUC G CAGAAGAU | 1557 | AUCUUCUG GCcgaaagGCGaGuCaaGGuCu GACGCGGC | 9134 |
| 2514 | GGUACCUU G CUUUAAUC | 1558 | GAUUAAAG GCcgaaagGCGaGuCaaGGuCu AAGGUACC | 9135 |
| 2560 | AUUCAUUU G CAGGAGGA | 1560 | UCCUCCUG GCcgaaagGCGaGuCaaGGuCu AAAUGAAU | 9136 |
| 2641 | UUAACUAU G CCUGCUAG | 1563 | CUAGCAGG GCcgaaagGCGaGuCaaGGuCu AUAGUUAA | 9137 |
| 2645 | CUAUGCCU G CUAGGUUU | 1564 | AAACCUAG GCcgaaagGCGaGuCaaGGuCu AGGCAUAG | 9138 |
| 2677 | AAAUAUUU G CCCUUAGA | 1565 | UCUAAGGG GCcgaaagGCGaGuCaaGGuCu AAAUAUUU | 9139 |
| 2740 | UUCCAGAC G CGACAUUA | 1566 | UAAUGUCG GCcgaaagGCGaGuCaaGGuCu GUCUGGAA | 9140 |
| 2804 | CACGUAGC G CCUCAUUU | 1568 | AAAUGAGG GCcgaaagGCGaGuCaaGGuCu GCUACGUC | 9141 |
| 2814 | CUCAUUUU G CGGGUCAC | 1569 | GUGACCCG GCcgaaagGCGaGuCaaGGuCu AAAAUGAG | 9142 |
| 2946 | UGGACCCU G CAUUCAAA | 1572 | UUUGAAUG GCcgaaagGCGaGuCaaGGuCu AGGGUCCA | 9143 |
| 2990 | CUCAACCC G CACAAGGA | 1573 | UCCUUGUG GCcgaaagGCGaGuCaaGGuCu GGGUUGAG | 9144 |
| 3012 | GGCCGGAC G CCAACAAG | 1574 | CUUGUUGG GCcgaaagGCGaGuCaaGGuCu GUCCGGCC | 9145 |
| 3090 | GCCCUCAC G CUCAGGGC | 1575 | GCCCUGAG GCcgaaagGCGaGuCaaGGuCu GUGAGGGC | 9146 |
| 3113 | ACAACUGU G CCAGCAGC | 1576 | GCUGCUGG GCcgaaagGCGaGuCaaGGuCu ACAGUUGU | 9147 |
| 3132 | CUCCUCCU G CCUCCACC | 1577 | GGUGGAGG GCcgaaagGCGaGuCaaGGuCu AGGAGGAG | 9148 |
| 51 | AGGGCCCU G UACUUUCC | 1578 | GGAAAGUA GCcgaaagGCGaGuCaaGGuCu AGGGCCCU | 9149 |
| 106 | AGAAUACU G UCUCUGCC | 1579 | GGCAGAGA GCcgaaagGCGaGuCaaGGuCu AGUAUUCU | 9150 |
| 148 | GGGACCCU G UACCGAAC | 1580 | GUUCGGUA GCcgaaagGCGaGuCaaGGuCu AGGGUCCC | 9151 |
| 198 | CUGCUCGU G UUACAGGC | 1581 | GCCUGUAA GCcgaaagGCGaGuCaaGGuCu ACGAGCAG | 9152 |
| 219 | UUUUUCUU G UUGACAAA | 1582 | UUUGUCAA GCcgaaagGCGaGuCaaGGuCu AAGAAAAA | 9153 |
| 297 | ACACCCGU G UGUCUUGG | 1583 | CCAAGACA GCcgaaagGCGaGuCaaGGuCu ACGGGUGU | 9154 |
| 299 | ACCCGUGU G UCUUGGCC | 1584 | GGCCAAGA GCcgaaagGCGaGuCaaGGuCu ACACGGGU | 9155 |
| 347 | ACCAACCU G UUGUCCUC | 1585 | GAGGACAA GCcgaaagGCGaGuCaaGGuCu AGGUUGGU | 9156 |
| 350 | AACCUGUU G UCCUCCAA | 1586 | UUGGAGGA GCcgaaagGCGaGuCaaGGuCu AACAGGUU | 9157 |
| 362 | UCCAAUUU G UCCUGGUU | 1587 | AACCAGGA GCcgaaagGCGaGuCaaGGuCu AAAUUGGA | 9158 |
| 381 | CGCUGGAU G UGUCUGCG | 1588 | CGCAGACA GCcgaaagGCGaGuCaaGGuCu AUCCAGCG | 9159 |
| 383 | CUGGAUGU G UCUGCGGC | 1589 | GCCGCAGA GCcgaaagGCGaGuCaaGGuCu ACAUCCAG | 9160 |
| 438 | AUCUUCUU G UUGGUUCU | 1590 | AGAACCAA GCcgaaagGCGaGuCaaGGuCu AAGAAGAU | 9161 |
| 465 | CAAGGUAU G UUGCCCGU | 1591 | ACGGGCAA GCcgaaagGCGaGuCaaGGuCu AUACCUUG | 9162 |
| 476 | GCCCGUUU G UCCUCUAA | 1592 | UUAGAGGA GCcgaaagGCGaGuCaaGGuCu AAACGGGC | 9163 |
| 555 | ACCUCUAU G UUUCCCUC | 1593 | GAGGGAAA GCcgaaagGCGaGuCaaGGuCu AUAGAGGU | 9164 |
| 566 | UCCCUCAU G UUGCUGUA | 1594 | UACAGCAA GCcgaaagGCGaGuCaaGGuCu AUGAGGGA | 9165 |
| 572 | AUGUUGCU G UACAAAAC | 1595 | GUUUUGUA GCcgaaagGCGaGuCaaGGuCu AGCAACAU | 9166 |
| 602 | CUGCACCU G UAUUCCCA | 1596 | UGGGAAUA GCcgaaagGCGaGuCaaGGuCu AGGUGCAG | 9167 |
| 694 | UGCCAUUU G UUCAGUGG | 1597 | CCACUGAA GCcgaaagGCGaGuCaaGGuCu AAAUGGCA | 9168 |
| 724 | CCCCCACU G UCUGGCUU | 1598 | AAGCCAGA GCcgaaagGCGaGuCaaGGuCu AGUGGGGG | 9169 |
| 750 | UGGAUGAU G UGGUUUUG | 1599 | CAAAACCA GCcgaaagGCGaGuCaaGGuCu AUCAUCCA | 9170 |
| 771 | CCAAGUCU G UACAACAU | 1600 | AUGUUGUA GCcgaaagGCGaGuCaaGGuCu AGACUUGG | 9171 |
| 801 | AUGCCGCU G UUACCAAU | 1601 | AUUGGUAA GCcgaaagGCGaGuCaaGGuCu AGCGGCAU | 9172 |
| 818 | UUUCUUUU G UCUUUGGG | 1602 | CCCAAAGA GCcgaaagGCGaGuCaaGGuCu AAAAGAAA | 9173 |
| 888 | UGGGAUAU G UAAUUGGG | 1603 | CCCAAUUA GCcgaaagGCGaGuCaaGGuCu AUAUCCCA | 9174 |
| 927 | AACAUAUU G UACAAAAA | 1604 | UUUUUGUA GCcgaaagGCGaGuCaaGGuCu AAUAUGUU | 9175 |
| 944 | AUCAAAAU G UGUUUUAG | 1605 | CUAAAACA GCcgaaagGCGaGuCaaGGuCu AUUUUGAU | 9176 |
| 946 | CAAAAUGU G UUUUAGGA | 1606 | UCCUAAAA GCcgaaagGCGaGuCaaGGuCu ACAUUUUG | 9177 |
| 963 | AACUUCCU G UAAACAGG | 1607 | CCUGUUUA GCcgaaagGCGaGuCaaGGuCu AGGAAGUU | 9178 |
| 991 | GAAAGUAU G UCAACGAA | 1608 | UUCGUUGA GCcgaaagGCGaGuCaaGGuCu AUACUUUC | 9179 |
| 1002 | AACGAAUU G UGGGUCUU | 1609 | AAGACCCA GCcgaaagGCGaGuCaaGGuCu AAUUCGUU | 9180 |
| 1039 | CACGCAAU G UGGAUAUU | 1610 | AAUAUCCA GCcgaaagGCGaGuCaaGGuCu AUUGCGUG | 9181 |
| 1137 | AACAGUAU G UGAACCUU | 1611 | AAGGUUCA GCcgaaagGCGaGuCaaGGuCu AUACUGUU | 9182 |
| 1184 | UGCCAAGU G UUUGCUGA | 1612 | UCAGCAAA GCcgaaagGCGaGuCaaGGuCu ACUUGGCA | 9183 |
| 1251 | GAACCUUU G UGUCUCCU | 1613 | AGGAGACA GCcgaaagGCGaGuCaaGGuCu AAAGGUUC | 9184 |
| 1253 | ACCUUUGU G UCUCCUCU | 1614 | AGAGGAGA GCcgaaagGCGaGuCaaGGuCu ACAAAGGU | 9185 |
| 1294 | AGCCGCUU G UUUUGCUC | 1615 | GAGCAAAA GCcgaaagGCGaGuCaaGGuCu AAGCGGCU | 9186 |
| 1344 | ACAAUUCU G UCGUGCUC | 1616 | GAGCACGA GCcgaaagGCGaGuCaaGGuCu AGAAUUGU | 9187 |
| 1390 | GCUAGGCU G UGCUGCCA | 1617 | UGGCAGCA GCcgaaagGCGaGuCaaGGuCu AGCCUAGC | 9188 |
| 1425 | CGUCCUUU G UUUACGUC | 1618 | GACGUAAA GCcgaaagGCGaGuCaaGGuCu AAAGGACG | 9189 |
| 1508 | CGCCUAUU G UACCGACC | 1619 | GGUCGGUA GCcgaaagGCGaGuCaaGGuCu AAUAGGCG | 9190 |
| 1557 | CCCCGUCU G UGCCUUCU | 1620 | AGAAGGCA GCcgaaagGCGaGuCaaGGuCu AGACGGGG | 9191 |
| 1581 | CGGACCGU G UGCACUUC | 1621 | GAAGUGCA GCcgaaagGCGaGuCaaGGuCu ACGGUCCG | 9192 |
| 1684 | UCAGCAAU G UCAACGAC | 1622 | GUCGUUGA GCcgaaagGCGaGuCaaGGuCu AUUGCUGA | 9193 |
| 1719 | CAAAGACU G UGUGUUUA | 1623 | UAAACACA GCcgaaagGCGaGuCaaGGuCu AGUCUUUG | 9194 |
| 1721 | AAGACUGU G UGUUUAAU | 1624 | AUUAAACA GCcgaaagGCGaGuCaaGGuCu ACAGUCUU | 9195 |
| 1723 | GACUGUGU G UUUAAUGA | 1625 | UCAUUAAA GCcgaaagGCGaGuCaaGGuCu ACACAGUC | 9196 |
| 1772 | AGGUCUUU G UACUAGGA | 1626 | UCCUAGUA GCcgaaagGCGaGuCaaGGuCu AAAGACCU | 9197 |
| 1785 | AGGAGGCU G UAGGCAUA | 1627 | UAUGCCUA GCcgaaagGCGaGuCaaGGuCu AGCCUCCU | 9198 |
| 1801 | AAAUUGGU G UGUUCACC | 1628 | GGUGAACA GCcgaaagGCGaGuCaaGGuCu ACCAAUUU | 9199 |
| 1803 | AUUGGUGU G UUCACCAG | 1629 | CUGGUGAA GCcgaaagGCGaGuCaaGGuCu ACACCAAU | 9200 |
| 1850 | CAUCUCAU G UUCAUGUC | 1630 | GACAUGAA GCcgaaagGCGaGuCaaGGuCu AUGAGAUG | 9201 |
| 1856 | AUGUUCAU G UCCUACUG | 1631 | CAGUAGGA GCcgaaagGCGaGuCaaGGuCu AUGAACAU | 9202 |
| 1864 | GUCCUACU G UUCAAGCC | 1632 | GGCUUGAA GCcgaaagGCGaGuCaaGGuCu AGUAGGAC | 9203 |
| 1881 | UCCAAGCU G UGCCUUGG | 1633 | CCAAGGCA GCcgaaagGCGaGuCaaGGuCu AGCUUGGA | 9204 |
| 1939 | GAGCUUCU G UGGAGUUA | 1634 | UAACUCCA GCcgaaagGCGaGuCaaGGuCu AGAAGCUC | 9205 |
| 2013 | UCUGCUCU G UAUCGGGG | 1635 | CCCCGAUA GCcgaaagGCGaGuCaaGGuCu AGAGCAGA | 9206 |
| 2045 | GGAACAUU G UUCACCUC | 1636 | GAGGUGAA GCcgaaagGCGaGuCaaGGuCu AAUGUUCC | 9207 |
| 2082 | GCUAUUCU G UGUUGGGG | 1637 | CCCCAACA GCcgaaagGCGaGuCaaGGuCu AGAAUAGC | 9208 |
| 2084 | UAUUCUGU G UUGGGGUG | 1638 | CACCCCAA GCcgaaagGCGaGuCaaGGuCu ACAGAAUA | 9209 |
| 2167 | UCAGCUAU G UCAACGUU | 1639 | AACGUUGA GCcgaaagGCGaGuCaaGGuCu AUAGCUGA | 9210 |
| 2205 | CAACUAUU G UGGUUUCA | 1640 | UGAAACCA GCcgaaagGCGaGuCaaGGuCu AAUAGUUG | 9211 |
| 2222 | CAUUUCCU G UCUUACUU | 1641 | AAGUAAGA GCcgaaagGCGaGuCaaGGuCu AGGAAAUG | 9212 |
| 2245 | GAGAAACU G UUCUUGAA | 1642 | UUCAAGAA GCcgaaagGCGaGuCaaGGuCu AGUUUCUC | 9213 |
| 2262 | UAUUUGGU G UCUUUUGG | 1643 | CCAAAAGA GCcgaaagGCGaGuCaaGGuCu ACCAAAUA | 9214 |
| 2274 | UUUGGAGU G UGGAUUCG | 1644 | CGAAUCCA GCcgaaagGCGaGuCaaGGuCu ACUCCAAA | 9215 |
| 2344 | AAACUACU G UUGUUAGA | 1645 | UCUAACAA GCcgaaagGCGaGuCaaGGuCu AGUAGUUU | 9216 |
| 2347 | CUACUGUU G UUAGACGA | 1646 | UCGUCUAA GCcgaaagGCGaGuCaaGGuCu AACAGUAG | 9217 |
| 2450 | AUCUCAAU G UUAGUAUU | 1647 | AAUACUAA GCcgaaagGCGaGuCaaGGuCu AUUGAGAU | 9218 |
| 2573 | AGGACAUU G UUGAUAGA | 1648 | UCUAUCAA GCcgaaagGCGaGuCaaGGuCu AAUGUCCU | 9219 |
| 2583 | UGAUAGAU G UAAGCAAU | 1649 | AUUGCUUA GCcgaaagGCGaGuCaaGGuCu AUCUAUCA | 9220 |
| 2594 | AGCAAUUU G UGGGGCCC | 1650 | GGGCCCCA GCcgaaagGCGaGuCaaGGuCu AAAUUGCU | 9221 |
| 2663 | AUCCCAAU G UUACUAAA | 1651 | UUUAGUAA GCcgaaagGCGaGuCaaGGuCu AUUGGGAU | 9222 |
| 2717 | CAGAGUAU G UAGUUAAU | 1652 | AUUAACUA GCcgaaagGCGaGuCaaGGuCu AUACUCUG | 9223 |
| 2901 | AUCUUUCU G UCCCCAAU | 1653 | AUUGGGGA GCcgaaagGCGaGuCaaGGuCu AGAAAGAU | 9224 |
| 3071 | GGGGGACU G UUGGGGUG | 1654 | CACCCCAA GCcgaaagGCGaGuCaaGGuCu AGUCCCCC | 9225 |
| 3111 | UCACAACU G UGCCAGCA | 1655 | UGCUGGCA GCcgaaagGCGaGuCaaGGuCu AGUUGUGA | 9226 |
| 40 | AUCCCAGA G UCAGGGCC | 1656 | GGCCCUGA GCcgaaagGCGaGuCaaGGuCu UCUGGGAU | 9227 |
| 46 | GAGUCAGG G CCCUGUAC | 1657 | GUACAGGG GCcgaaagGCGaGuCaaGGuCu CCUGACUC | 9228 |
| 65 | UCCUGCUG G UGGCUCCA | 1658 | UGGAGCCA GCcgaaagGCGaGuCaaGGuCu CAGCAGGA | 9229 |
| 68 | UGCUGGUG G CUCCAGUU | 1659 | AACUGGAG GCcgaaagGCGaGuCaaGGuCu CACCAGCA | 9230 |
| 74 | UGGCUCCA G UUCAGGAA | 1660 | UUCCUGAA GCcgaaagGCGaGuCaaGGuCu UGGAGCCA | 9231 |
| 85 | CAGGAACA G UGAGCCCU | 1661 | AGGGCUCA GCcgaaagGCGaGuCaaGGuCu UGUUCCUG | 9232 |
| 89 | AACAGUGA G CCCUGCUC | 1662 | GAGCAGGG GCcgaaagGCGaGuCaaGGuCu UCACUGUU | 9233 |
| 120 | GCCAUAUC G UCAAUCUU | 1663 | AAGAUUGA GCcgaaagGCGaGuCaaGGuCu GAUAUGGC | 9234 |
| 196 | CCCUGCUC G UGUUACAG | 1664 | CUGUAACA GCcgaaagGCGaGuCaaGGuCu GAGCAGGG | 9235 |
| 205 | UGUUACAG G CGGGGUUU | 1665 | AAACCCCG GCcgaaagGCGaGuCaaGGuCu CUGUAACA | 9236 |
| 210 | CAGGCGGG G UUUUUCUU | 1666 | AAGAAAAA GCcgaaagGCGaGuCaaGGuCu CCCGCCUG | 9237 |
| 248 | ACCACAGA G UCUAGACU | 1667 | AGUCUAGA GCcgaaagGCGaGuCaaGGuCu UCUGUGGU | 9238 |
| 258 | CUAGACUC G UGGUGGAC | 1668 | GUCCACCA GCcgaaagGCGaGuCaaGGuCu GAGUCUAG | 9239 |
| 261 | GACUCGUG G UGGACUUC | 1669 | GAAGUCCA GCcgaaagGCGaGuCaaGGuCu CACGAGUC | 9240 |
| 295 | GAACACCC G UGUGUCUU | 1670 | AAGACACA GCcgaaagGCGaGuCaaGGuCu GGGUGUUC | 9241 |
| 305 | GUGUCUUG G CCAAAAUU | 1671 | AAUUUUGG GCcgaaagGCGaGuCaaGGuCu CAAGACAC | 9242 |
| 318 | AAUUCGCA G UCCCAAAU | 1672 | AUUUGGGA GCcgaaagGCGaGuCaaGGuCu UGCGAAUU | 9243 |
| 332 | AAUCUCCA G UCACUCAC | 1673 | GUGAGUGA GCcgaaagGCGaGuCaaGGuCu UGGAGAUU | 9244 |
| 368 | UUGUCCUG G UUAUCGCU | 1674 | AGCGAUAA GCcgaaagGCGaGuCaaGGuCu CAGGACAA | 9245 |
| 390 | UGUCUGCG G CGUUUUAU | 1675 | AUAAAACG GCcgaaagGCGaGuCaaGGuCu CGCAGACA | 9246 |
| 392 | UCUGCGGC G UUUUAUCA | 1676 | UGAUAAAA GCcgaaagGCGaGuCaaGGuCu GCCGCAGA | 9247 |
| 442 | UCUUGUUG G UUCUUCUG | 1677 | CAGAAGAA GCcgaaagGCGaGuCaaGGuCu CAACAAGA | 9248 |
| 461 | CUAUCAAG G UAUGUUGC | 1678 | GCAACAUA GCcgaaagGCGaGuCaaGGuCu CUUGAUAG | 9249 |
| 472 | UGUUGCCC G UUUGUCCU | 1679 | AGGACAAA GCcgaaagGCGaGuCaaGGuCu GGGCAACA | 9250 |
| 506 | AACAACCA G CACCGGAC | 1680 | GUCCGGUG GCcgaaagGCGaGuCaaGGuCu UGGUUGUU | 9251 |
| 625 | CAUCUUGG G CUUUCGCA | 1681 | UGCGAAAG GCcgaaagGCGaGuCaaGGuCu CCAAGAUG | 9252 |
| 648 | CUAUGGGA G UGGGCCUC | 1682 | GAGGCCCA GCcgaaagGCGaGuCaaGGuCu UCCCAUAG | 9253 |
| 652 | GGGAGUGG G CCUCAGUC | 1683 | GACUGAGG GCcgaaagGCGaGuCaaGGuCu CCACUCCC | 9254 |
| 658 | GGGCCUCA G UCCGUUUC | 1684 | GAAACGGA GCcgaaagGCGaGuCaaGGuCu UGAGGCCC | 9255 |
| 662 | CUCAGUCC G UUUCUCUU | 1685 | AAGAGAAA GCcgaaagGCGaGuCaaGGuCu GGACUGAG | 9256 |
| 672 | UUCUCUUG G CUCAGUUU | 1686 | AAACUGAG GCcgaaagGCGaGuCaaGGuCu CAAGAGAA | 9257 |
| 677 | UUGGCUCA G UUUACUAG | 1687 | CUAGUAAA GCcgaaagGCGaGuCaaGGuCu UGAGCCAA | 9258 |
| 685 | GUUUACUA G UGCCAUUU | 1688 | AAAUGGCA GCcgaaagGCGaGuCaaGGuCu UAGUAAAC | 9259 |
| 699 | UUUGUUCA G UGGUUCGU | 1689 | ACGAACCA GCcgaaagGCGaGuCaaGGuCu UGAACAAA | 9260 |
| 702 | GUUCAGUG G UUCGUAGG | 1690 | CCUACGAA GCcgaaagGCGaGuCaaGGuCu CACUGAAC | 9261 |
| 706 | AGUGGUUC G UAGGGCUU | 1691 | AAGCCCUA GCcgaaagGCGaGuCaaGGuCu GAACCACU | 9262 |
| 711 | UUCGUAGG G CUUUCCCC | 1692 | GGGGAAAG GCcgaaagGCGaGuCaaGGuCu CCUACGAA | 9263 |
| 729 | ACUGUCUG G CUUUCAGU | 1693 | ACUGAAAG GCcgaaagGCGaGuCaaGGuCu CAGACAGU | 9264 |
| 736 | GGCUUUCA G UUAUAUGG | 1694 | CCAUAUAA GCcgaaagGCGaGuCaaGGuCu UGAAAGCC | 9265 |
| 753 | AUGAUGUG G UUUUGGGG | 1695 | CCCCAAAA GCcgaaagGCGaGuCaaGGuCu CACAUCAU | 9266 |
| 762 | UUUUGGGG G CCAAGUCU | 1696 | AGACUUGG GCcgaaagGCGaGuCaaGGuCu CCCCAAAA | 9267 |
| 767 | GGGGCCAA G UCUGUACA | 1697 | UGUACAGA GCcgaaagGCGaGuCaaGGuCu UUGGCCCC | 9268 |
| 785 | CAUCUUGA G UCCCUUUA | 1698 | UAAAGGGA GCcgaaagGCGaGuCaaGGuCu UCAAGAUG | 9269 |
| 826 | GUCUUUGG G UAUACAUU | 1699 | AAUGUAUA GCcgaaagGCGaGuCaaGGuCu CCAAAGAC | 9270 |
| 898 | AAUUGGGA G UUGGGGCA | 1700 | UGCCCCAA GCcgaaagGCGaGuCaaGGuCu UCCCAAUU | 9271 |
| 904 | GAGUUGGG G CACAUUGC | 1701 | GCAAUGUG GCcgaaagGCGaGuCaaGGuCu CCCAACUC | 9272 |
| 971 | GUAAACAG G CCUAUUGA | 1702 | UCAAUAGG GCcgaaagGCGaGuCaaGGuCu CUGUUUAC | 9273 |
| 987 | AUUGGAAA G UAUGUCAA | 1703 | UUGACAUA GCcgaaagGCGaGuCaaGGuCu UUUCCAAU | 9274 |
| 1006 | AAUUGUGG G UCUUUUGG | 1704 | CCAAAAGA GCcgaaagGCGaGuCaaGGuCu CCACAAUU | 9275 |
| 1016 | CUUUUGGG G UUUGCCGC | 1705 | GCGGCAAA GCcgaaagGCGaGuCaaGGuCu CCCAAAAG | 9276 |
| 1080 | GCAUACAA G CAAAACAG | 1706 | CUGUUUUG GCcgaaagGCGaGuCaaGGuCu UUGUAUGC | 9277 |
| 1089 | CAAAACAG G CUUUUACU | 1707 | AGUAAAAG GCcgaaagGCGaGuCaaGGuCu CUGUUUUG | 9278 |
| 1116 | CUUACAAG G CCUUUCUA | 1708 | UAGAAAGG GCcgaaagGCGaGuCaaGGuCu CUUGUAAG | 9279 |
| 1126 | CUUUCUAA G UAAACAGU | 1709 | ACUGUUUA GCcgaaagGCGaGuCaaGGuCu UUAGAAAG | 9280 |
| 1133 | AGUAAACA G UAUGUGAA | 1710 | UUCACAUA GCcgaaagGCGaGuCaaGGuCu UGUUUACU | 9281 |
| 1152 | UUUACCCC G UUGCUCGG | 1711 | CCGAGCAA GCcgaaagGCGaGuCaaGGuCu GGGGUAAA | 9282 |
| 1160 | GUUGCUCG G CAACGGCC | 1712 | GGCCGUUG GCcgaaagGCGaGuCaaGGuCu CGAGCAAC | 9283 |
| 1166 | CGGCAACG G CCUGGUCU | 1713 | AGACCAGG GCcgaaagGCGaGuCaaGGuCu CGUUGCCG | 9284 |
| 1171 | ACGGCCUG G UCUAUGCC | 1714 | GGCAUAGA GCcgaaagGCGaGuCaaGGuCu CAGGCCGU | 9285 |
| 1182 | UAUGCCAA G UGUUUGCU | 1715 | AGCAAACA GCcgaaagGCGaGuCaaGGuCu UUGGCAUA | 9286 |
| 1207 | CCCCACUG G UUGGGGCU | 1716 | AGCCCCAA GCcgaaagGCGaGuCaaGGuCu CAGUGGGG | 9287 |
| 1213 | UGGUUGGG G CUUGGCCA | 1717 | UGGCCAAG GCcgaaagGCGaGuCaaGGuCu CCCAACCA | 9288 |
| 1218 | GGGGCUUG G CCAUAGGC | 1718 | GCCUAUGG GCcgaaagGCGaGuCaaGGuCu CAAGCCCC | 9289 |
| 1225 | CCCCAUAG G CCAUCAGC | 1719 | GCUGAUGG GCcgaaagGCGaGuCaaGGuCu CUAUGGCC | 9290 |
| 1232 | GGCCAUCA G CGCAUGCG | 1720 | CGCAUGCG GCcgaaagGCGaGuCaaGGuCu UGAUGGCC | 9291 |
| 1240 | GCGCAUGC G UGGAACCU | 1721 | AGGUUCCA GCcgaaagGCGaGuCaaGGuCu GCAUGCGC | 9292 |
| 1287 | AACUCCUA G CCGCUUGU | 1722 | ACAAGCGG GCcgaaagGCGaGuCaaGGuCu UAGGAGUU | 9293 |
| 1306 | UGCUCGCA G CAGGUCUG | 1723 | CAGACCUG GCcgaaagGCGaGuCaaGGuCu UGCGAGCA | 9294 |
| 1310 | CGCAGCAG G UCUGGGGC | 1724 | GCCCCAGA GCcgaaagGCGaGuCaaGGuCu CUGCUGCG | 9295 |
| 1317 | GGUCUGGG G CAAAACUC | 1725 | GAGUUUUG GCcgaaagGCGaGuCaaGGuCu CCCAGACC | 9296 |
| 1347 | AUUCUGUC G UGCUCUCC | 1726 | GGAGAGCA GCcgaaagGCGaGuCaaGGuCu GACAGAAU | 9297 |
| 1379 | UUUCCAUG G CUGCUAGG | 1727 | CCUAGCAG GCcgaaagGCGaGuCaaGGuCu CAUGGAAA | 9298 |
| 1387 | GCUGCUAG G CUGUGCUG | 1728 | CAGCACAG GCcgaaagGCGaGuCaaGGuCu CUAGCAGC | 9299 |
| 1418 | CGCGGGAC G UCCUUUGU | 1729 | ACAAAGGA GCcgaaagGCGaGuCaaGGuCu GUCCCGCG | 9300 |
| 1431 | UUGUUUAC G UCCCGUCG | 1730 | CGACGGGA GCcgaaagGCGaGuCaaGGuCu GUAAACAA | 9301 |
| 1436 | UACGUCCC G UCGGCGCU | 1731 | AGCGCCGA GCcgaaagGCGaGuCaaGGuCu GGGACGUA | 9302 |
| 1440 | UCCCGUCG G CGCUGAAU | 1732 | AUUCAGCG GCcgaaagGCGaGuCaaGGuCu CGACGGGA | 9303 |
| 1471 | CUCCCGGG G CCGCUUGG | 1733 | CCAAGCGG GCcgaaagGCGaGuCaaGGuCu CCCGGGAG | 9304 |
| 1481 | CGCUUGGG G CUCUACCG | 1734 | CGGUAGAG GCcgaaagGCGaGuCaaGGuCu CCCAAGCG | 9305 |
| 1517 | UACCGACC G UCCACGGG | 1735 | CCCGUGGA GCcgaaagGCGaGuCaaGGuCu GGUCGGUA | 9306 |
| 1526 | UCCACGGG G CGCACCUC | 1736 | GAGGUGCG GCcgaaagGCGaGuCaaGGuCu CCCGUGGA | 9307 |
| 1553 | GACUCCCC G UCUGUGCC | 1737 | GGCACAGA GCcgaaagGCGaGuCaaGGuCu GGGGAGUC | 9308 |
| 1579 | GCCGGACC G UGUGCACU | 1738 | AGUGCACA GCcgaaagGCGaGuCaaGGuCu GGUCCGGC | 9309 |
| 1605 | CUCUGCAC G UCGCAUGG | 1739 | CCAUGCGA GCcgaaagGCGaGuCaaGGuCu GUGCAGAG | 9310 |
| 1622 | AGACCACC G UGAACGCC | 1740 | GGCGUUCA GCcgaaagGCGaGuCaaGGuCu GGUGGUCU | 9311 |
| 1649 | UGCCCAAG G UCUUGCAU | 1741 | AUGCAAGA GCcgaaagGCGaGuCaaGGuCu CUUGGGCA | 9312 |
| 1679 | GACUUUCA G CAAUGUCA | 1742 | UGACAUUG GCcgaaagGCGaGuCaaGGuCu UGAAAGUC | 9313 |
| 1703 | ACCUUGAG G CAUACUUC | 1743 | GAAGUAUG GCcgaaagGCGaGuCaaGGuCu CUCAAGGU | 9314 |
| 1732 | UUUAAUGA G UGGGAGGA | 1744 | UCCUCCCA GCcgaaagGCGaGuCaaGGuCu UCAUUAAA | 9315 |
| 1741 | UGGGAGGA G UUGGGGGA | 1745 | UCCCCCAA GCcgaaagGCGaGuCaaGGuCu UCCUCCCA | 9316 |
| 1754 | GGGAGGAG G UUAGGUUA | 1746 | UAACCUAA GCcgaaagGCGaGuCaaGGuCu CUCCUCCC | 9317 |
| 1759 | GAGGUUAG G UUAAAGGU | 1747 | ACCUUUAA GCcgaaagGCGaGuCaaGGuCu CUAACCUC | 9318 |
| 1766 | GGUUAAAG G UCUUUGUA | 1748 | UACAAAGA GCcgaaagGCGaGuCaaGGuCu CUUUAACC | 9319 |
| 1782 | ACUAGGAG G CUGUAGGC | 1749 | GCCUACAG GCcgaaagGCGaGuCaaGGuCu CUCCUAGU | 9320 |
| 1789 | GGCUGUAG G CAUAAAUU | 1750 | AAUUUAUG GCcgaaagGCGaGuCaaGGuCu CUACAGCC | 9321 |
| 1799 | AUAAAUUG G UGUGUUCA | 1751 | UGAACACA GCcgaaagGCGaGuCaaGGuCu CAAUUUAU | 9322 |
| 1811 | GUUCACCA G CACCAUGC | 1752 | GCAUGGUG GCcgaaagGCGaGuCaaGGuCu UGGUGAAC | 9323 |
| 1870 | CUGUUCAA G CCUCCAAG | 1753 | CUUGGAGG GCcgaaagGCGaGuCaaGGuCu UUGAACAG | 9324 |
| 1878 | GCCUCCAA G CUGUGCCU | 1754 | AGGCACAG GCcgaaagGCGaGuCaaGGuCu UUGGAGGC | 9325 |
| 1890 | UGCCUUGG G UGGCUUUG | 1755 | CAAAGCCA GCcgaaagGCGaGuCaaGGuCu CCAAGGCA | 9326 |
| 1893 | CUUGGGUG G CUUUGGGG | 1756 | CCCCAAAG GCcgaaagGCGaGuCaaGGuCu CACCCAAG | 9327 |
| 1901 | GCUUUGGG G CAUGGACA | 1757 | UGUCCAUG GCcgaaagGCGaGuCaaGGuCu CCCAAAGC | 9328 |
| 1917 | AUUGACCC G UAUAAAGA | 1758 | UCUUUAUA GCcgaaagGCGaGuCaaGGuCu GGGUCAAU | 9329 |
| 1933 | AAUUUGGA G CUUCUGUG | 1759 | CACAGAAG GCcgaaagGCGaGuCaaGGuCu UCCAAAUU | 9330 |
| 1944 | UCUGUGGA G UUACUCUC | 1760 | GAGAGUAA GCcgaaagGCGaGuCaaGGuCu UCCACAGA | 9331 |
| 2023 | AUCGGGGG G CCUUAGAG | 1761 | CUCUAAGG GCcgaaagGCGaGuCaaGGuCu CCCCCGAU | 9332 |
| 2031 | GCCUUAGA G UCUCCGGA | 1762 | UCCGGAGA GCcgaaagGCGaGuCaaGGuCu UCUAAGGC | 9333 |
| 2062 | ACCAUACG G CACUCAGG | 1763 | CCUGAGUG GCcgaaagGCGaGuCaaGGuCu CGUAUGGU | 9334 |
| 2070 | GCACUCAG G CAAGCUAU | 1764 | AUAGCUUG GCcgaaagGCGaGuCaaGGuCu CUGAGUGC | 9335 |
| 2074 | UCAGGCAA G CUAUUCUG | 1765 | CAGAAUAG GCcgaaagGCGaGuCaaGGuCu UUGCCUGA | 9336 |
| 2090 | GUGUUGGG G UGAGUUGA | 1766 | UCAACUCA GCcgaaagGCGaGuCaaGGuCu CCCAACAC | 9337 |
| 2094 | UGGGGUGA G UUGAUGAA | 1767 | UUCAUCAA GCcgaaagGCGaGuCaaGGuCu UCACCCCA | 9338 |
| 2107 | UGAAUCUA G CCACCUGG | 1768 | CCAGGUGG GCcgaaagGCGaGuCaaGGuCu UAGAUUCA | 9339 |
| 2116 | CCACCUGG G UGGGAAGU | 1769 | ACUUCCCA GCcgaaagGCGaGuCaaGGuCu CCAGGUGG | 9340 |
| 2123 | GGUGGGAA G UAAUUUGG | 1770 | CCAAAUUA GCcgaaagGCGaGuCaaGGuCu UUCCCACC | 9341 |
| 2140 | AAGAUCCA G CAUCCAGG | 1771 | CCUGGAUG GCcgaaagGCGaGuCaaGGuCu UGGAUCUU | 9342 |
| 2155 | GGGAAUUA G UAGUCAGC | 1772 | GCUGACUA GCcgaaagGCGaGuCaaGGuCu UAAUUCCC | 9343 |
| 2158 | AAUUAGUA G UCAGCUAU | 1773 | AUAGCUGA GCcgaaagGCGaGuCaaGGuCu UACUAAUU | 9344 |
| 2162 | AGUAGUCA G CUAUGUCA | 1774 | UGACAUAG GCcgaaagGCGaGuCaaGGuCu UGACUACU | 9345 |
| 2173 | AUGUCAAC G UUAAUAUG | 1775 | CAUAUUAA GCcgaaagGCGaGuCaaGGuCu GUUGACAU | 9346 |
| 2183 | UAAUAUGG G CCUAAAAA | 1776 | UUUUUAGG GCcgaaagGCGaGuCaaGGuCu CCAUAUUA | 9347 |
| 2208 | CUAUUGUG G UUUCACAU | 1777 | AUGUGAAA GCcgaaagGCGaGuCaaGGuCu CACAAUAG | 9348 |
| 2235 | ACUUUUGG G CGAGAAAC | 1778 | GUUUCUCG GCcgaaagGCGaGuCaaGGuCu CCAAAAGU | 9349 |
| 2260 | AAUAUUUG G UGUCUUUU | 1779 | AAAAGACA GCcgaaagGCGaGuCaaGGuCu CAAAUAUU | 9350 |
| 2272 | CUUUUGGA G UGUGGAUU | 1780 | AAUCCACA GCcgaaagGCGaGuCaaGGuCu UCCAAAAG | 9351 |
| 2360 | ACGAAGAG G CAGGUCCC | 1781 | GGGACCUG GCcgaaagGCGaGuCaaGGuCu CUCUUCGU | 9352 |
| 2364 | AGAGGCAG G UCCCCUAG | 1782 | CUAGGGGA GCcgaaagGCGaGuCaaGGuCu CUGCCUCU | 9353 |
| 2403 | AGACGAAG G UCUCAAUC | 1783 | GAUUGAGA GCcgaaagGCGaGuCaaGGuCu CUUCGUCU | 9354 |
| 2417 | AUCGCCGC G UCGCAGAA | 1784 | UUCUGCGA GCcgaaagGCGaGuCaaGGuCu GCGGCGAU | 9355 |
| 2454 | CAAUGUUA G UAUUCCUU | 1785 | AAGGAAUA GCcgaaagGCGaGuCaaGGuCu UAACAUUG | 9356 |
| 2474 | CACAUAAG G UGGGAAAC | 1786 | GUUUCCCA GCcgaaagGCGaGuCaaGGuCu CUUAUGUG | 9357 |
| 2491 | UUUACGGG G CUUUAUUC | 1787 | GAAUAAAG GCcgaaagGCGaGuCaaGGuCu CCCGUAAA | 9358 |
| 2507 | CUUCUACG G UACCUUGC | 1788 | GCAAGGUA GCcgaaagGCGaGuCaaGGuCu CGUAGAAG | 9359 |
| 2530 | CCUAAAUG G CAAACUCC | 1789 | GGAGUUUG GCcgaaagGCGaGuCaaGGuCu CAUUUAGG | 9360 |
| 2587 | AGAUGUAA G CAAUUUGU | 1790 | ACAAAUUG GCcgaaagGCGaGuCaaGGuCu UUACAUCU | 9361 |
| 2599 | UUUGUGGG G CCCCUUAC | 1791 | GUAAGGGG GCcgaaagGCGaGuCaaGGuCu CCCACAAA | 9362 |
| 2609 | CCCUUACA G UAAAUGAA | 1792 | UUCAUUUA GCcgaaagGCGaGuCaaGGuCu UGUAAGGG | 9363 |
| 2650 | CCUGCUAG G UUUUAUCC | 1793 | GGAUAAAA GCcgaaagGCGaGuCaaGGuCu CUAGCAGG | 9364 |
| 2701 | AUCAAACC G UAUUAUCC | 1794 | GGAUAAUA GCcgaaagGCGaGuCaaGGuCu GGUUUGAU | 9365 |
| 2713 | UAUCCAGA G UAUGUAGU | 1795 | ACUACAUA GCcgaaagGCGaGuCaaGGuCu UCUGGAUA | 9366 |
| 2720 | AGUAUGUA G UUAAUCAU | 1796 | AUGAUUAA GCcgaaagGCGaGuCaaGGuCu UACAUACU | 9367 |
| 2768 | UUUGGAAG G CGGGGAUC | 1797 | GAUCCCCG GCcgaaagGCGaGuCaaGGuCu CUUCCAAA | 9368 |
| 2791 | AAAAGAGA G UCCACACG | 1798 | CGUGUGGA GCcgaaagGCGaGuCaaGGuCu UCUCUUUU | 9369 |
| 2799 | GUCCACAC G UAGCGCCU | 1799 | AGGCGCUA GCcgaaagGCGaGuCaaGGuCu GUGUGGAC | 9370 |
| 2802 | CACACGUA G CGCCUCAU | 1800 | AUGAGGCG GCcgaaagGCGaGuCaaGGuCu UACGUGUG | 9371 |
| 2818 | UUUUGCGG G UCACCAUA | 1801 | UAUGGUGA GCcgaaagGCGaGuCaaGGuCu CCGCAAAA | 9372 |
| 2848 | GAUCUACA G CAUGGGAG | 1802 | CUCCCAUG GCcgaaagGCGaGuCaaGGuCu UGUAGAUC | 9373 |
| 2857 | CAUGGGAG G UUGGUCUU | 1803 | AAGACCAA GCcgaaagGCGaGuCaaGGuCu CUCCCAUG | 9374 |
| 2861 | GGAGGUUG G UCUUCCAA | 1804 | UUGGAAGA GCcgaaagGCGaGuCaaGGuCu CAACCUCC | 9375 |
| 2881 | UCGAAAAG G CAUGGGGA | 1805 | UCCCCAUG GCcgaaagGCGaGuCaaGGuCu CUUUUCGA | 9376 |
| 2936 | GAUCAUCA G UUGGACCC | 1806 | GGGUCCAA GCcgaaagGCGaGuCaaGGuCu UGAUGAUC | 9377 |
| 2955 | CAUUCAAA G CCAACUCA | 1807 | UGAGUUGG GCcgaaagGCGaGuCaaGGuCu UUUGAAUG | 9378 |
| 2964 | CCAACUCA G UAAAUCCA | 1808 | UGGAUUUA GCcgaaagGCGaGuCaaGGuCu UGAGUUGG | 9379 |
| 3005 | GACAACUG G CCGGACGC | 1809 | GCGUCCGG GCcgaaagGCGaGuCaaGGuCu CAGUUGUC | 9380 |
| 3021 | CCAACAAG G UGGGAGUG | 1810 | CACUCCCA GCcgaaagGCGaGuCaaGGuCu CUUGUUGG | 9381 |
| 3027 | AGGUGGGA G UGGGAGCA | 1811 | UGCUCCCA GCcgaaagGCGaGuCaaGGuCu UCCCACCU | 9382 |
| 3033 | GAGUGGGA G CAUUCGGG | 1812 | CCCGAAUG GCcgaaagGCGaGuCaaGGuCu UCCCACUC | 9383 |
| 3041 | GCAUUCGG G CCAGGGUU | 1813 | AACCCUGG GCcgaaagGCGaGuCaaGGuCu CCGAAUGC | 9384 |
| 3047 | GGGCCAGG G UUCACCCC | 1814 | GGGGUGAA GCcgaaagGCGaGuCaaGGuCu CCUGGCCC | 9385 |
| 3077 | CUGUUGGG G UGGAGCCC | 1815 | GGGCUCCA GCcgaaagGCGaGuCaaGGuCu CCCAACAG | 9386 |
| 3082 | GGGGUGGA G CCCUCACG | 1816 | CGUGAGGG GCcgaaagGCGaGuCaaGGuCu UCCACCCC | 9387 |
| 3097 | CGCUCAGG G CCUACUCA | 1817 | UGAGUAGG GCcgaaagGCGaGuCaaGGuCu CCUGAGCG | 9388 |
| 3117 | CUGUGCCA G CAGCUCCU | 1818 | AGGAGCUG GCcgaaagGCGaGuCaaGGuCu UGGCACAG | 9389 |
| 3120 | UGCCAGCA G CUCCUCCU | 1819 | AGGAGGAG GCcgaaagGCGaGuCaaGGuCu UGCUGGCA | 9390 |
| 3146 | ACCAAUCG G CAGUCAGG | 1820 | CCUGACUG GCcgaaagGCGaGuCaaGGuCu CGAUUGGU | 9391 |
| 3149 | AAUCGGCA G UCAGGAAG | 1821 | CUUCCUGA GCcgaaagGCGaGuCaaGGuCu UGCCGAUU | 9392 |
| 3158 | UCAGGAAG G CAGCCUAC | 1822 | GUAGGCUG GCcgaaagGCGaGuCaaGGuCu CUUCCUGA | 9393 |
| 3161 | GGAAGGCA G CCUACUCC | 1823 | GGAGUAGG GCcgaaagGCGaGuCaaGGuCu UGCCUUCC | 9394 |
| 3204 | AUCCUCAG G CCAUGCAG | 1824 | CUGCAUGG GCcgaaagGCGaGuCaaGGuCu CUGAGGAU | 9395 |

Input Sequence = AF100308.
Cut Site = YG/M or UG/U.
Stem Length = 8.
Core Sequence = GCcgaaagGCGaGuCaaGGuCu
AF100308 (Hepatitis B virus strain 2-18, 3215 bp)

TABLE IX

HUMAN HBV DNAZYME AND SUBSTRATE SEQUENCE

| | | | | |
|---|---|---|---|---|
| Pos | Substrate | Seq ID | DNAzyme | Seq ID |

| | | | | |
|---|---|---|---|---|
| 508 | CAACCAGC A CCGGACCA | 833 | TGGTCCGG GGCTAGCTACAACGA GCTGGTTG | 9396 |
| 1632 | GAACGCCC A CAGGAACC | 1096 | GGTTCCTG GGCTAGCTACAACGA GGGCGTTC | 9397 |
| 2992 | CAACCCGC A CAAGGACA | 1376 | TGTCCTTG GGCTAGCTACAACGA GCGGGTTG | 9398 |
| 61 | ACUUUCCU G CUGGUGGC | 1448 | GCCACCAG GGCTAGCTACAACGA AGGAAAGT | 9399 |
| 94 | UGAGCCCU G CUCAGAAU | 1450 | ATTCTGAG GGCTAGCTACAACGA AGGGCTCA | 9400 |
| 112 | CUGUCUCU G CCAUAUCG | 1451 | CGATATGG GGCTAGCTACAACGA AGAGACAG | 9401 |
| 169 | AGAACAUC G CAUCAGGA | 1454 | TCCTGATG GGCTAGCTACAACGA GATGTTCT | 9402 |
| 192 | GGACCCCU G CUCGUGUU | 1455 | AACACGAG GGCTAGCTACAACGA AGGGGTCC | 9403 |
| 315 | CAAAAUUC G CAGUCCCA | 1457 | TGGGACTG GGCTAGCTACAACGA GAATTTTG | 9404 |
| 374 | UGGUUAUC G CUGGAUGU | 1458 | ACATCCAG GGCTAGCTACAACGA GATAACCA | 9405 |
| 387 | AUGUGUCU G CGGCGUUU | 1459 | AAACGCCG GGCTAGCTACAACGA AGACACAT | 9406 |
| 410 | CUUCCUCU G CAUCCUGC | 1460 | GCAGGATG GGCTAGCTACAACGA AGAGGAAG | 9407 |
| 417 | UGCAUCCU G CUGCUAUG | 1461 | CATAGCAG GGCTAGCTACAACGA AGGATGCA | 9408 |
| 420 | AUCCUGCU G CUAUGCCU | 1462 | AGGCATAG GGCTAGCTACAACGA AGCAGGAT | 9409 |
| 425 | GCUGCUAU G CCUCAUCU | 1463 | AGATGAGG GGCTAGCTACAACGA ATAGCAGC | 9410 |
| 468 | GGUAUGUU G CCCGUUUG | 1464 | CAAACGGG GGCTAGCTACAACGA AACATACC | 9411 |
| 518 | CGGACCAU G CAAAACCU | 1465 | AGGTTTTG GGCTAGCTACAACGA ATGGTCCG | 9412 |
| 527 | CAAAACCU G CACAACUC | 1466 | GAGTTGTG GGCTAGCTACAACGA AGGTTTTG | 9413 |
| 538 | CAACUCCU G CUCAAGGA | 1467 | TCCTTGAG GGCTAGCTACAACGA AGGAGTTG | 9414 |
| 569 | CUCAUGUU G CUGUACAA | 1468 | TTGTACAG GGCTAGCTACAACGA AACATGAG | 9415 |
| 596 | CGGAAACU G CACCUGUA | 1469 | TACAGGTG GGCTAGCTACAACGA AGTTTCCG | 9416 |
| 631 | GGGCUUUC G CAAAAUAC | 1470 | GTATTTTG GGCTAGCTACAACGA GAAAGCCC | 9417 |
| 687 | UUACUAGU G CCAUUUGU | 1471 | ACAAATGG GGCTAGCTACAACGA ACTAGTAA | 9418 |
| 795 | CCCUUUAU G CCGCUGUU | 1474 | AACAGCGG GGCTAGCTACAACGA ATAAAGGG | 9419 |
| 798 | UUUAUGCC G CUGUUACC | 1475 | GGTAACAG GGCTAGCTACAACGA GGCATAAA | 9420 |
| 911 | GGCACAUU G CCACAGGA | 1476 | TCCTGTGG GGCTAGCTACAACGA AATGTGCC | 9421 |
| 1020 | UGGGGUUU G CCGCCCCU | 1479 | AGGGGCGG GGCTAGCTACAACGA AAACCCCA | 9422 |
| 1023 | GGUUUGCC G CCCCUUUC | 1480 | GAAAGGGG GGCTAGCTACAACGA GGCAAACC | 9423 |
| 1034 | CCUUUCAC G CAAUGUGG | 1481 | CCACATTG GGCTAGCTACAACGA GTGAAAGG | 9424 |
| 1050 | GAUAUUCU G CUUUAAUG | 1482 | CATTAAAG GGCTAGCTACAACGA AGAATATC | 9425 |
| 1058 | GCUUUAAU G CCUUUAUA | 1483 | TATAAAGG GGCTAGCTACAACGA ATTAAAGC | 9426 |
| 1068 | CUUUAUAU G CAUGCAUA | 1484 | TATGCATG GGCTAGCTACAACGA ATATAAAG | 9427 |
| 1072 | AUAUGCAU G CAUACAAG | 1485 | CTTGTATG GGCTAGCTACAACGA ATGCATAT | 9428 |
| 1103 | ACUUUCUC G CCAACUUA | 1486 | TAAGTTGG GGCTAGCTACAACGA GAGAAAGT | 9429 |
| 1155 | ACCCCGUU G CUCGGCAA | 1488 | TTGCCGAG GGCTAGCTACAACGA AACGGGGT | 9430 |
| 1177 | UGGUCUAU G CCAAGUGU | 1489 | ACACTTGG GGCTAGCTACAACGA ATAGACCA | 9431 |
| 1188 | AAGUGUUU G CUGACGCA | 1490 | TGCGTCAG GGCTAGCTACAACGA AAACACTT | 9432 |
| 1194 | UUGCUGAC G CAACCCCC | 1492 | GGGGGTTG GGCTAGCTACAACGA GTCAGCAA | 9433 |
| 1234 | CCAUCAGC G CAUGCGUG | 1493 | CACGCATG GGCTAGCTACAACGA GCTGATGG | 9434 |
| 1238 | CAGCGCAU G CGUGGAAC | 1494 | GTTCCACG GGCTAGCTACAACGA ATGCGCTG | 9435 |
| 1262 | UCUCCUCU G CCGAUCCA | 1495 | TGGATCGG GGCTAGCTACAACGA AGAGGAGA | 9436 |
| 1275 | UCCAUACC G CGGAACUC | 1497 | GAGTTCCG GGCTAGCTACAACGA GGTATGGA | 9437 |
| 1290 | UCCUAGCC G CUUGUUUU | 1498 | AAAACAAG GGCTAGCTACAACGA GGCTAGGA | 9438 |
| 1299 | CUUGUUUU G CUCGCAGC | 1499 | GCTGCGAG GGCTAGCTACAACGA AAAACAAG | 9439 |
| 1303 | UUUUGCUC G CAGCAGGU | 1500 | ACCTGCTG GGCTAGCTACAACGA GAGCAAAA | 9440 |
| 1349 | UCUGUCGU G CUCUCCCG | 1502 | CGGGAGAG GGCTAGCTACAACGA ACGACAGA | 9441 |
| 1357 | GCUCUCCC G CAAAUAUA | 1503 | TATATTTG GGCTAGCTACAACGA GGGAGAGC | 9442 |
| 1382 | CCAUGGCU G CUAGGCUG | 1504 | CAGCCTAG GGCTAGCTACAACGA AGCCATGG | 9443 |
| 1392 | UAGGCUGU G CUGCCAAC | 1505 | GTTGGCAG GGCTAGCTACAACGA ACAGCCTA | 9444 |
| 1395 | GCUGUGCU G CCAACUGG | 1506 | CCAGTTGG GGCTAGCTACAACGA AGCACAGC | 9445 |
| 1411 | GAUCCUAC G CGGGACGU | 1507 | ACGTCCCG GGCTAGCTACAACGA GTAGGATC | 9446 |
| 1442 | CCGUCGGC G CUGAAUCC | 1508 | GGATTCAG GGCTAGCTACAACGA GCCGACGG | 9447 |
| 1452 | UGAAUCCC G CGGACGAC | 1510 | GTCGTCCG GGCTAGCTACAACGA GGGATTCA | 9448 |
| 1474 | CCGGGGCC G CUUGGGGC | 1512 | GCCCCAAG GGCTAGCTACAACGA GGCCCCGG | 9449 |
| 1489 | GCUCUACC G CCCGCUUC | 1513 | GAAGCGGG GGCTAGCTACAACGA GGTAGAGC | 9450 |
| 1493 | UACCGCCC G CUUCUCCG | 1514 | CGGAGAAG GGCTAGCTACAACGA GGGCGGTA | 9451 |
| 1501 | GCUUCUCC G CCUAUUGU | 1515 | ACAATAGG GGCTAGCTACAACGA GGAGAAGC | 9452 |
| 1528 | CACGGGGC G CACCUCUC | 1517 | CAGAGGTG GGCTAGCTACAACGA GCCCCGTG | 9453 |
| 1542 | CUCUUUAC G CGGACUCC | 1518 | GGACTCCG GGCTAGCTACAACGA GTAAAGAG | 9454 |
| 1559 | CCGUCUGU G CCUUCUCA | 1519 | TGAGAAGG GGCTAGCTACAACGA ACAGACGG | 9455 |
| 1571 | UCUCAUCU G CCGGACCG | 1520 | CGGTCCGG GGCTAGCTACAACGA AGATGAGA | 9456 |
| 1583 | GACCGUGU G CACUUCGC | 1521 | GCGAAGTG GGCTAGCTACAACGA ACACGGTC | 9457 |
| 1590 | UGCACUUC G CUUCACCU | 1522 | AGGTGAAG GGCTAGCTACAACGA GAAGTGCA | 9458 |
| 1601 | UCACCUCU G CACGUCGC | 1523 | GCGACGTG GGCTAGCTACAACGA AGAGGTGA | 9459 |
| 1608 | UGCACGUC G CAUGGAGA | 1524 | TCTCCATG GGCTAGCTACAACGA GACGTGCA | 9460 |
| 1628 | CCGUGAAC G CCCACAGG | 1526 | CCTGTGGG GGCTAGCTACAACGA GTTCACGG | 9461 |
| 1642 | AGGAACCU G CCCAAGGU | 1527 | ACCTTGGG GGCTAGCTACAACGA AGGTTCCT | 9462 |
| 1654 | AAGGUCUU G CAUAAGAG | 1528 | CTCTTATG GGCTAGCTACAACGA AAGACCTT | 9463 |
| 1818 | AGCACCAU G CAACUUUU | 1533 | AAAAGTTG GGCTAGCTACAACGA ATGGTGCT | 9464 |
| 1835 | UCACCUCU G CCUAAUCA | 1534 | TGATTAGG GGCTAGCTACAACGA AGAGGTGA | 9465 |
| 1883 | CAAGCUGU G CCUUGGGU | 1535 | ACCCAAGG GGCTAGCTACAACGA ACAGCTTG | 9466 |
| 1959 | UCUUUUUU G CCUUCUGA | 1537 | TCAGAAGG GGCTAGCTACAACGA AAAAAAGA | 9467 |
| 2002 | UCGACACC G CCUCUGCU | 1541 | AGCAGAGG GGCTAGCTACAACGA GGTGTCGA | 9468 |
| 2008 | CCGCCUCU G CUCUGUAU | 1542 | ATACAGAG GGCTAGCTACAACGA AGAGGCGG | 9469 |
| 2282 | GUGGAUUC G CACUCCUC | 1548 | GAGGAGTG GGCTAGCTACAACGA GAATCCAC | 9470 |
| 2293 | CUCCUCCU G CAUAUAGA | 1549 | TCTATATG GGCTAGCTACAACGA AGGAGGAG | 9471 |
| 2311 | CACCAAAU G CCCCUAUC | 1550 | GATAGGGG GGCTAGCTACAACGA ATTTGGTG | 9472 |
| 2388 | ACUCCCUC G CCUCGCAG | 1552 | CTGCGAGG GGCTAGCTACAACGA GAGGGAGT | 9473 |
| 2393 | CUCGCCUC G CAGACGAA | 1553 | TTCGTCTG GGCTAGCTACAACGA GAGGCGAG | 9474 |
| 2412 | UCUCAAUC G CCGCGUCG | 1555 | CGACGCGG GGCTAGCTACAACGA GATTGAGA | 9475 |
| 2415 | CAAUCGCC G CGUCGCAG | 1556 | CTGCGACG GGCTAGCTACAACGA GGCGATTG | 9476 |
| 2420 | GCCGCGUC G CAGAAGAU | 1557 | ATCTTCTG GGCTAGCTACAACGA GACGCGGC | 9477 |
| 2514 | GGUACCUU G CUUUAAUC | 1558 | GATTAAAG GGCTAGCTACAACGA AAGGTACC | 9478 |
| 2560 | AUUCAUUU G CAGGAGGA | 1560 | TCCTCCTG GGCTAGCTACAACGA AAATGAAT | 9479 |
| 2641 | UUAACUAU G CCUGCUAG | 1563 | CTAGCAGG GGCTAGCTACAACGA ATAGTTAA | 9480 |
| 2645 | CUAUGCCU G CUAGGUUU | 1564 | AAACCTAG GGCTAGCTACAACGA AGGCATAG | 9481 |
| 2677 | AAAUAUUU G CCCUUAGA | 1565 | TCTAAGGG GGCTAGCTACAACGA AAATATTT | 9482 |
| 2740 | UUCCAGAC G CGACAUUA | 1566 | TAATGTCG GGCTAGCTACAACGA GTCTGGAA | 9483 |
| 2804 | CACGUAGC G CCUCAUUU | 1568 | AAATGAGG GGCTAGCTACAACGA GCTACGTG | 9484 |
| 2814 | CUCAUUUU G CGGGUCAC | 1569 | GTGACCCG GGCTAGCTACAACGA AAAATGAG | 9485 |
| 2946 | UGGACCCU G CAUUCAAA | 1572 | TTTGAATG GGCTAGCTACAACGA AGGGTCCA | 9486 |
| 2990 | CUCAACCC G CACAAGGA | 1573 | TCCTTGTG GGCTAGCTACAACGA GGGTTGAG | 9487 |
| 3012 | GGCCGGAC G CCAACAAG | 1574 | CTTGTTGG GGCTAGCTACAACGA GTCCGGCC | 9488 |
| 3090 | GCCCUCAC G CUCAGGGC | 1575 | GCCCTGAG GGCTAGCTACAACGA GTGAGGGC | 9489 |
| 3113 | ACAACUGU G CCAGCAGC | 1576 | GCTGCTGG GGCTAGCTACAACGA ACAGTTGT | 9490 |
| 3132 | CUCCUCCU G CCUCCACC | 1577 | GGTGGAGG GGCTAGCTACAACGA AGGAGGAG | 9491 |
| 51 | AGGGCCCU G UACUUUCC | 1578 | GGAAAGTA GGCTAGCTACAACGA AGGGCCCT | 9492 |
| 106 | AGAAUACU G UCUCUGCC | 1579 | GGCAGAGA GGCTAGCTACAACGA AGTATTCT | 9493 |
| 148 | GGGACCCU G UACCGAAC | 1580 | GTTCGGTA GGCTAGCTACAACGA AGGGTCCC | 9494 |
| 198 | CUGCUCGU G UUACAGGC | 1581 | GCCTGTAA GGCTAGCTACAACGA ACGAGCAG | 9495 |
| 219 | UUUUUCUU G UUGACAAA | 1582 | TTTGTCAA GGCTAGCTACAACGA AAGAAAAA | 9496 |
| 297 | ACACCCGU G UGUCUUGG | 1583 | CCAAGACA GGCTAGCTACAACGA ACGGGTGT | 9497 |
| 299 | ACCCGUGU G UCUUGGCC | 1584 | GGCCAAGA GGCTAGCTACAACGA ACACGGGT | 9498 |
| 347 | ACCAACCU G UUGUCCUC | 1585 | GAGGACAA GGCTAGCTACAACGA AGGTTGGT | 9499 |
| 350 | AACCUGUU G UCCUCCAA | 1586 | TTGGAGGA GGCTAGCTACAACGA AACAGGTT | 9500 |
| 362 | UCCAAUUU G UCCUGGUU | 1587 | AACCAGGA GGCTAGCTACAACGA AAATTGGA | 9501 |
| 381 | CGCUGGAU G UGUCUGCG | 1588 | CGCAGACA GGCTAGCTACAACGA ATCCAGCG | 9502 |
| 383 | CUGGAUGU G UCUGCGGC | 1589 | GCCGCAGA GGCTAGCTACAACGA ACATCCAG | 9503 |
| 438 | AUCUUCUU G UUGGUUCU | 1590 | AGAACCAA GGCTAGCTACAACGA AAGAAGAT | 9504 |
| 465 | CAAGGUAU G UUGCCCGU | 1591 | ACGGGCAA GGCTAGCTACAACGA ATACCTTG | 9505 |
| 476 | GCCCGUUU G UCCUCUAA | 1592 | TTAGAGGA GGCTAGCTACAACGA AAACGGGC | 9506 |
| 555 | ACCUCUAU G UUUCCCUC | 1593 | GAGGGAAA GGCTAGCTACAACGA ATAGAGGT | 9507 |
| 566 | UCCCUCAU G UUGCUGUA | 1594 | TACAGCAA GGCTAGCTACAACGA ATGAGGGA | 9508 |
| 572 | AUGUUGCU G UACAAAAC | 1595 | GTTTTGTA GGCTAGCTACAACGA AGCAACAT | 9509 |
| 602 | CUGCACCU G UAUUCCCA | 1596 | TGGGAATA GGCTAGCTACAACGA AGGTGCAG | 9510 |
| 694 | UGCCAUUU G UUCAGUGG | 1597 | CCACTGAA GGCTAGCTACAACGA AAATGGCA | 9511 |
| 724 | CCCCCACU G UCUGGCUU | 1598 | AAGCCAGA GGCTAGCTACAACGA AGTGGGGG | 9512 |
| 750 | UGGAUGAU G UGGUUUUG | 1599 | CAAAACCA GGCTAGCTACAACGA ATCATCCA | 9513 |
| 771 | CCAAGUCU G UACAACAU | 1600 | ATGTTGTA GGCTAGCTACAACGA AGACTTGG | 9514 |
| 801 | AUGCCGCU G UUACCAAU | 1601 | ATTGGTAA GGCTAGCTACAACGA AGCGGCAT | 9515 |
| 818 | UUUCUUUU G UCUUUGGG | 1602 | CCCAAAGA GGCTAGCTACAACGA AAAACAAA | 9516 |
| 888 | UGGGAUAU G UAAUUGGG | 1603 | CCCAATTA GGCTAGCTACAACGA ATATCCCA | 9517 |
| 927 | AACAUAUU G UACAAAAA | 1604 | TTTTTGTA GGCTAGCTACAACGA AATATGTT | 9518 |
| 944 | AUCAAAAU G UGUUUUAG | 1605 | CTAAAACA GGCTAGCTACAACGA ATTTTGAT | 9519 |
| 946 | CAAAAUGU G UUUUAGGA | 1606 | TCCTAAAA GGCTAGCTACAACGA ACATTTTG | 9520 |
| 963 | AACUUCCU G UAAACAGG | 1607 | CCTGTTTA GGCTAGCTACAACGA AGGAAGTT | 9521 |
| 991 | GAAAGUAU G UCAACGAA | 1608 | TTCGTTGA GGCTAGCTACAACGA ATACTTTC | 9522 |
| 1002 | AACGAAUU G UGGGUCUU | 1609 | AAGACCCA GGCTAGCTACAACGA AATTCGTT | 9523 |
| 1039 | CACGCAAU G UGGAUAUU | 1610 | AATATCCA GGCTAGCTACAACGA ATTGCGTG | 9524 |
| 1137 | AACAGUAU G UGAACCUU | 1611 | AAGGTTCA GGCTAGCTACAACGA ATACTGTT | 9525 |
| 1184 | UGCCAAGU G UUUGCUGA | 1612 | TCAGCAAA GGCTAGCTACAACGA ACTTGGCA | 9526 |
| 1251 | GAACCUUU G UGUCUCCU | 1613 | AGGAGACA GGCTAGCTACAACGA AAAGGTTC | 9527 |
| 1253 | ACCUUUGU G UCUCCUCU | 1614 | AGAGGAGA GGCTAGCTACAACGA ACAAAGGT | 9528 |
| 1294 | AGCCGCUU G UUUUGCUC | 1615 | GAGCAAAA GGCTAGCTACAACGA AAGCGGCT | 9529 |
| 1344 | ACAAUUCU G UCGUGCUC | 1616 | GAGCACGA GGCTAGCTACAACGA AGAATTGT | 9530 |
| 1390 | GCUAGGCU G UGCUGCCA | 1617 | TGGCAGCA GGCTAGCTACAACGA AGCCTAGC | 9531 |
| 1425 | CGUCCUUU G UUUACGUC | 1618 | GACGTAAA GGCTAGCTACAACGA AAAGGACG | 9532 |
| 1508 | CGCCUAUU G UACCGACC | 1619 | GGTCGGTA GGCTAGCTACAACGA AATAGGCG | 9533 |
| 1557 | CCCCGUCU G UGCCUUCU | 1620 | AGAAGGCA GGCTAGCTACAACGA AGACGGGG | 9534 |
| 1581 | CGGACCGU G UGCACUUC | 1621 | GAAGTGCA GGCTAGCTACAACGA ACGGTCCG | 9535 |
| 1684 | UCAGCAAU G UCAACGAC | 1622 | GTCGTTGA GGCTAGCTACAACGA ATTGCTGA | 9536 |
| 1719 | CAAAGACU G UGUGUUUA | 1623 | TAAACACA GGCTAGCTACAACGA AGTCTTTG | 9537 |
| 1721 | AAGACUGU G UGUUUAAU | 1624 | ATTAAACA GGCTAGCTACAACGA ACAGTCTT | 9538 |
| 1723 | GACUGUGU G UUUAAUGA | 1625 | TCATTAAA GGCTAGCTACAACGA ACACAGTC | 9539 |
| 1772 | AGGUCUUU G UACUAGGA | 1626 | TCCTAGTA GGCTAGCTACAACGA AAAGACCT | 9540 |
| 1785 | AGGAGGCU G UAGGCAUA | 1627 | TATGCCTA GGCTAGCTACAACGA AGCCTCCT | 9541 |
| 1801 | AAAUUGGU G UGUUCACC | 1628 | GGTGAACA GGCTAGCTACAACGA ACCAATTT | 9542 |
| 1803 | AUUGGUGU G UUCACCAG | 1629 | CTGGTGAA GGCTAGCTACAACGA ACACCAAT | 9543 |
| 1850 | CAUCUCAU G UUCAUGUC | 1630 | GACATGAA GGCTAGCTACAACGA ATGAGATG | 9544 |
| 1856 | AUGUUCAU G UCCUACUG | 1631 | CAGTAGGA GGCTAGCTACAACGA ATGAACAT | 9545 |
| 1864 | GUCCUACU G UUCAAGCC | 1632 | GGCTTGAA GGCTAGCTACAACGA AGTAGGAC | 9546 |
| 1881 | UCCAAGCU G UGCCUUGG | 1633 | CCAAGGCA GGCTAGCTACAACGA AGCTTGGA | 9547 |
| 1939 | GAGCUUCU G UGGAGUUA | 1634 | TAACTCCA GGCTAGCTACAACGA AGAAGCTC | 9548 |
| 2013 | UCUGCUCU G UAUCGGGG | 1635 | CCCCGATA GGCTAGCTACAACGA AGAGCAGA | 9549 |
| 2045 | GGAACAUU G UUCACCUC | 1636 | GAGGTGAA GGCTAGCTACAACGA AATGTTCC | 9550 |
| 2082 | GCUAUUCU G UGUUGGGG | 1637 | CCCCAACA GGCTAGCTACAACGA AGAATAGC | 9551 |
| 2084 | UAUUCUGU G UUGGGGUG | 1638 | CACCCCAA GGCTAGCTACAACGA ACAGAATA | 9552 |
| 2167 | UCAGCUAU G UCAACGUU | 1639 | AACGTTGA GGCTAGCTACAACGA ATAGCTGA | 9553 |
| 2205 | CAACUAUU G UGGUUUCA | 1640 | TGAAACCA GGCTAGCTACAACGA AATAGTTG | 9554 |
| 2222 | CAUUUCCU G UCUUACUU | 1641 | AAGTAAGA GGCTAGCTACAACGA AGGAAATG | 9555 |
| 2245 | GAGAAACU G UUCUUGAA | 1642 | TTCAAGAA GGCTAGCTACAACGA AGTTTCTC | 9556 |
| 2262 | UAUUUGGU G UCUUUUGG | 1643 | CCAAAAGA GGCTAGCTACAACGA ACCAAATA | 9557 |
| 2274 | UUUGGAGU G UGGAUUCG | 1644 | CGAATCCA GGCTAGCTACAACGA ACTCCAAA | 9558 |
| 2344 | AAACUACU G UUGUUAGA | 1645 | TCTAACAA GGCTAGCTACAACGA AGTAGTTT | 9559 |
| 2347 | CUACUGUU G UUAGACGA | 1646 | TCGTCTAA GGCTAGCTACAACGA AACAGTAG | 9560 |
| 2450 | AUCUCAAU G UUAGUAUU | 1647 | AATACTAA GGCTAGCTACAACGA ATTGAGAT | 9561 |
| 2573 | AGGACAUU G UUGAUAGA | 1648 | TCTATCAA GGCTAGCTACAACGA AATGTCCT | 9562 |
| 2583 | UGAUAGAU G UAAGCAAU | 1649 | ATTGCTTA GGCTAGCTACAACGA ATCTATCA | 9563 |
| 2594 | AGCAAUUU G UGGGGCCC | 1650 | GGGCCCCA GGCTAGCTACAACGA AAATTGCT | 9564 |
| 2663 | AUCCCAAU G UUACUAAA | 1651 | TTTAGTAA GGCTAGCTACAACGA ATTGGGAT | 9565 |
| 2717 | CAGAGUAU G UAGUUAAU | 1652 | ATTAACTA GGCTAGCTACAACGA ATACTCTG | 9566 |
| 2901 | AUCUUUCU G UCCCCAAU | 1653 | ATTGGGGA GGCTAGCTACAACGA AGAAAGAT | 9567 |
| 3071 | GGGGGACU G UUGGGGUG | 1654 | CACCCCAA GGCTAGCTACAACGA AGTCCCCC | 9568 |
| 3111 | UCACAACU G UGCCAGCA | 1655 | TGCTGGCA GGCTAGCTACAACGA AGTTGTGA | 9569 |
| 40 | AUCCCAGA G UCAGGGCC | 1656 | GGCCCTGA GGCTAGCTACAACGA TCTGGGAT | 9570 |
| 46 | GAGUCAGG G CCCUGUAC | 1657 | GTACAGGG GGCTAGCTACAACGA CCTGACTC | 9571 |
| 65 | UCCUGCUG G UGGCUCCA | 1658 | TGGAGCCA GGCTAGCTACAACGA CAGCAGGA | 9572 |
| 68 | UGCUGGUG G CUCCAGUU | 1659 | AACTGGAG GGCTAGCTACAACGA CACCAGCA | 9573 |
| 74 | UGGCUCCA G UUCAGGAA | 1660 | TTCCTGAA GGCTAGCTACAACGA TGGAGCCA | 9574 |
| 85 | CAGGAACA G UGAGCCCU | 1661 | AGGGCTCA GGCTAGCTACAACGA TGTTCCTG | 9575 |
| 89 | AACAGUGA G CCCUGCUC | 1662 | GAGCAGGG GGCTAGCTACAACGA TCACTGTT | 9576 |
| 120 | GCCAUAUC G UCAAUCUU | 1663 | AAGATTGA GGCTAGCTACAACGA GATATGGC | 9577 |
| 196 | CCCUGCUC G UGUUACAG | 1664 | CTGTAACA GGCTAGCTACAACGA GAGCAGGG | 9578 |
| 205 | UGUUACAG G CGGGGUUU | 1665 | AAACCCCG GGCTAGCTACAACGA CTGTAACA | 9579 |
| 210 | CAGGCGGG G UUUUUCUU | 1666 | AAGAAAAA GGCTAGCTACAACGA CCCGCCTG | 9580 |
| 248 | ACCACAGA G UCUAGACU | 1667 | AGTCTAGA GGCTAGCTACAACGA TCTGTGGT | 9581 |
| 258 | CUAGACUC G UGGUGGAC | 1668 | GTCCACCA GGCTAGCTACAACGA GAGTCTAG | 9582 |
| 261 | GACUCGUG G UGGACUUC | 1669 | GAAGTCCA GGCTAGCTACAACGA CACGAGTC | 9583 |
| 295 | GAACACCC G UGUGUCUU | 1670 | AAGACACA GGCTAGCTACAACGA GGGTGTTC | 9584 |
| 305 | GUGUCUUG G CCAAAAUU | 1671 | AATTTTGG GGCTAGCTACAACGA CAAGACAC | 9585 |
| 318 | AAUUCGCA G UCCCAAAU | 1672 | ATTTGGGA GGCTAGCTACAACGA TGCGAATT | 9586 |
| 332 | AAUCUCCA G UCACUCAC | 1673 | GTGAGTGA GGCTAGCTACAACGA TGGAGATT | 9587 |
| 368 | UUGUCCUG G UUAUCGCU | 1674 | AGCGATAA GGCTAGCTACAACGA CAGGACAA | 9588 |
| 390 | UGUCUGCG G CGUUUUAU | 1675 | ATAAAACG GGCTAGCTACAACGA CGCAGACA | 9589 |
| 392 | UCUGCGGC G UUUUAUCA | 1676 | TGATAAAA GGCTAGCTACAACGA GCCGCAGA | 9590 |
| 442 | UCUUGUUG G UUCUUCUG | 1677 | CAGAAGAA GGCTAGCTACAACGA CAACAAGA | 9591 |
| 461 | CUAUCAAG G UAUGUUGC | 1678 | GCAACATA GGCTAGCTACAACGA CTTGATAG | 9592 |
| 472 | UGUUGCCC G UUUGUCCU | 1679 | AGGACAAA GGCTAGCTACAACGA GGGCAACA | 9593 |
| 506 | AACAACCA G CACCGGAC | 1680 | GTCCGGTG GGCTAGCTACAACGA TGGTTGTT | 9594 |
| 625 | CAUCUUGG G CUUUCGCA | 1681 | TGCGAAAG GGCTAGCTACAACGA CCAAGATG | 9595 |
| 648 | CUAUGGGA G UGGGCCUC | 1682 | GAGGCCCA GGCTAGCTACAACGA TCCCATAG | 9596 |
| 652 | GGGAGUGG G CCUCAGUC | 1683 | GACTGAGG GGCTAGCTACAACGA CCACTCCC | 9597 |
| 658 | GGGCCUCA G UCCGUUUC | 1684 | GAAACGGA GGCTAGCTACAACGA TGAGGCCC | 9598 |
| 662 | CUCAGUCC G UUUCUCUU | 1685 | AAGAGAAA GGCTAGCTACAACGA GGACTGAG | 9599 |
| 672 | UUCUCUUG G CUCAGUUU | 1686 | AAACTGAG GGCTAGCTACAACGA CAAGAGAA | 9600 |
| 677 | UUGGCUCA G UUUACUAG | 1687 | CTAGTAAA GGCTAGCTACAACGA TGAGCCAA | 9601 |
| 685 | GUUUACUA G UGCCAUUU | 1688 | AAATGGCA GGCTAGCTACAACGA TAGTAAAC | 9602 |
| 699 | UUUGUUCA G UGGUUCGU | 1689 | ACGAACCA GGCTAGCTACAACGA TGAACAAA | 9603 |
| 702 | GUUCAGUG G UUCGUAGG | 1690 | CCTACGAA GGCTAGCTACAACGA CACTGAAC | 9604 |
| 706 | AGUGGUUC G UAGGGCUU | 1691 | AAGCCCTA GGCTAGCTACAACGA GAACCACT | 9605 |
| 711 | UUCGUAGG G CUUUCCCC | 1692 | GGGGAAAG GGCTAGCTACAACGA CCTACGAA | 9606 |
| 729 | ACUGUCUG G CUUUCAGU | 1693 | ACTGAAAG GGCTAGCTACAACGA CAGACAGT | 9607 |
| 736 | GGCUUUCA G UUAUAUGG | 1694 | CCATATAA GGCTAGCTACAACGA TGAAAGCC | 9608 |
| 753 | AUGAUGUG G UUUUGGGG | 1695 | CCCCAAAA GGCTAGCTACAACGA CACATCAT | 9609 |
| 762 | UUUUGGGG G CCAAGUCU | 1696 | AGACTTGG GGCTAGCTACAACGA CCCCAAAA | 9610 |
| 767 | GGGGCCAA G UCUGUACA | 1697 | TGTACAGA GGCTAGCTACAACGA TTGGCCCC | 9611 |
| 785 | CAUCUUGA G UCCCUUUA | 1698 | TAAAGGGA GGCTAGCTACAACGA TCAAGATG | 9612 |
| 826 | GUCUUUGG G UAUACAUU | 1699 | AATGTATA GGCTAGCTACAACGA CCAAAGAC | 9613 |
| 898 | AAUUGGGA G UUGGGGCA | 1700 | TGCCCCAA GGCTAGCTACAACGA TCCCAATT | 9614 |
| 904 | GAGUUGGG G CACAUUGC | 1701 | GCAATGTG GGCTAGCTACAACGA CCCAACTC | 9615 |
| 971 | GUAAACAG G CCUAUUGA | 1702 | TCAATAGG GGCTAGCTACAACGA CTGTTTAC | 9616 |
| 987 | AUUGGAAA G UAUGUCAA | 1703 | TTGACATA GGCTAGCTACAACGA TTTCCAAT | 9617 |
| 1006 | AAUUGUGG G UCUUUUGG | 1704 | CCAAAAGA GGCTAGCTACAACGA CCACAATT | 9618 |
| 1016 | CUUUUGGG G UUUGCCGC | 1705 | GCGGCAAA GGCTAGCTACAACGA CCCAAAAG | 9619 |
| 1080 | GCAUACAA G CAAAACAG | 1706 | CTGTTTTG GGCTAGCTACAACGA TTGTATGC | 9620 |
| 1089 | CAAAACAG G CUUUUACU | 1707 | AGTAAAAG GGCTAGCTACAACGA CTGTTTTG | 9621 |
| 1116 | CUUACAAG G CCUUUCUA | 1708 | TAGAAAGG GGCTAGCTACAACGA CTTGTAAG | 9622 |
| 1126 | CUUUCUAA G UAAACAGU | 1709 | ACTGTTTA GGCTAGCTACAACGA TTAGAAAG | 9623 |
| 1133 | AGUAAACA G UAUGUGAA | 1710 | TTCACATA GGCTAGCTACAACGA TGTTTACT | 9624 |
| 1152 | UUUACCCC G UUGCUCGG | 1711 | CCGAGCAA GGCTAGCTACAACGA GGGGTAAA | 9625 |
| 1160 | GUUGCUCG G CAACGGCC | 1712 | GGCCGTTG GGCTAGCTACAACGA CGAGCAAC | 9626 |
| 1166 | CGGCAACG G CCUGGUCU | 1713 | AGACCAGG GGCTAGCTACAACGA CGTTGCCG | 9627 |
| 1171 | ACGGCCUG G UCUAUGCC | 1714 | GGCATAGA GGCTAGCTACAACGA CAGGCCGT | 9628 |
| 1182 | UAUGCCAA G UGUUUGCU | 1715 | AGCAAACA GGCTAGCTACAACGA TTGGCATA | 9629 |
| 1207 | CCCCACUG G UUGGGGCU | 1716 | AGCCCCAA GGCTAGCTACAACGA CAGTGGGG | 9630 |
| 1213 | UGGUUGGG G CUUGGCCA | 1717 | TGGCCAAG GGCTAGCTACAACGA CCCAACCA | 9631 |
| 1218 | GGGGCUUG G CCAUAGGC | 1718 | GCCTATGG GGCTAGCTACAACGA CAAGCCCC | 9632 |
| 1225 | GGCCAUAG G CCAUCAGC | 1719 | GCTGATGG GGCTAGCTACAACGA CTATGGCC | 9633 |
| 1232 | GGCCAUCA G CGCAUGCG | 1720 | CGCATGCG GGCTAGCTACAACGA TGATGGCC | 9634 |
| 1240 | GCGCAUGC G UGGAACCU | 1721 | AGGTTCCA GGCTAGCTACAACGA GCATGCGC | 9635 |
| 1287 | AACUCCUA G CCGCUUGU | 1722 | ACAAGCGG GGCTAGCTACAACGA TAGGAGTT | 9636 |
| 1306 | UGCUCGCA G CAGGUCUG | 1723 | CAGACCTG GGCTAGCTACAACGA TGCGAGCA | 9637 |
| 1310 | CGCAGCAG G UCUGGGGC | 1724 | GCCCCAGA GGCTAGCTACAACGA CTGCTGCG | 9638 |
| 1317 | GGUCUGGG G CAAAACUC | 1725 | GAGTTTTG GGCTAGCTACAACGA CCCAGACC | 9639 |
| 1347 | AUUCUGUC G UGCUCUCC | 1726 | GGAGAGCA GGCTAGCTACAACGA GACAGAAT | 9640 |
| 1379 | UUUCCAUG G CUGCUAGG | 1727 | CCTAGCAG GGCTAGCTACAACGA CATGGAAA | 9641 |
| 1387 | GCUGCUAG G CUGUGCUG | 1728 | CAGCACAG GGCTAGCTACAACGA CTAGCAGC | 9642 |
| 1418 | CGCGGGAC G UCCUUUGU | 1729 | ACAAAGGA GGCTAGCTACAACGA GTCCCGCG | 9643 |
| 1431 | UUGUUUAC G UCCCGUCG | 1730 | CGACGGGA GGCTAGCTACAACGA GTAAACAA | 9644 |
| 1436 | UACGUCCC G UCGGCGCU | 1731 | AGCGCCGA GGCTAGCTACAACGA GGGACGTA | 9645 |
| 1440 | UCCCGUCG G CGCUGAAU | 1732 | ATTCAGCG GGCTAGCTACAACGA CGACGGGA | 9646 |
| 1471 | CUCCCGGG G CCGCUUGG | 1733 | CCAAGCGG GGCTAGCTACAACGA CCCGGGAG | 9647 |
| 1481 | CGCUUGGG G CUCUACCG | 1734 | CGGTAGAG GGCTAGCTACAACGA CCCAAGCG | 9648 |
| 1517 | UACCGACC G UCCACGGG | 1735 | CCCGTGGA GGCTAGCTACAACGA GGTCGGTA | 9649 |
| 1526 | UCCACGGG G CGCACCUC | 1736 | GAGGTGCG GGCTAGCTACAACGA CCCGTGGA | 9650 |
| 1553 | GACUCCCC G UCUGUGCC | 1737 | GGCACAGA GGCTAGCTACAACGA GGGGAGTC | 9651 |
| 1579 | GCCGGACC G UGUGCACU | 1738 | AGTGCACA GGCTAGCTACAACGA GGTCCGGC | 9652 |
| 1605 | CUCUGCAC G UCGCAUGG | 1739 | CCATGCGA GGCTAGCTACAACGA GTGCAGAG | 9653 |
| 1622 | AGACCACC G UGAACGCC | 1740 | GGCGTTCA GGCTAGCTACAACGA GGTGGTCT | 9654 |
| 1649 | UGCCCAAG G UCUUGCAU | 1741 | ATGCAAGA GGCTAGCTACAACGA CTTGGGCA | 9655 |
| 1679 | GACUUUCA G CAAUGUCA | 1742 | TGACATTG GGCTAGCTACAACGA TGAAAGTC | 9656 |
| 1703 | ACCUUGAG G CAUACUUC | 1743 | GAAGTATG GGCTAGCTACAACGA CTCAAGGT | 9657 |
| 1732 | UUUAAUGA G UGGGAGGA | 1744 | TCCTCCCA GGCTAGCTACAACGA TCATTAAA | 9658 |
| 1741 | UGGGAGGA G UUGGGGGA | 1745 | TCCCCCAA GGCTAGCTACAACGA TCCTCCCA | 9659 |
| 1754 | GGGAGGAG G UUAGGUUA | 1746 | TAACCTAA GGCTAGCTACAACGA CTCCTCCC | 9660 |
| 1759 | GAGGUUAG G UUAAAGGU | 1747 | ACCTTTAA GGCTAGCTACAACGA CTAACCTC | 9661 |
| 1766 | GGUUAAAG G UCUUUGUA | 1748 | TACAAAGA GGCTAGCTACAACGA CTTTAACC | 9662 |
| 1782 | ACUAGGAG G CUGUAGGC | 1749 | GCCTACAG GGCTAGCTACAACGA CTCCTAGT | 9663 |
| 1789 | GGCUGUAG G CAUAAAUU | 1750 | AATTTATG GGCTAGCTACAACGA CTACAGCC | 9664 |
| 1799 | AUAAAUUG G UGUGUUCA | 1751 | TGAACACA GGCTAGCTACAACGA CAATTTAT | 9665 |
| 1811 | GUUCACCA G CACCAUGC | 1752 | GCATGGTG GGCTAGCTACAACGA TGGTGAAC | 9666 |
| 1870 | CUGUUCAA G CCUCCAAG | 1753 | CTTGGAGG GGCTAGCTACAACGA TTGAACAG | 9667 |
| 1878 | GCCUCCAA G CUGUGCCU | 1754 | AGGCACAG GGCTAGCTACAACGA TTGGAGGC | 9668 |
| 1890 | UGCCUUGG G UGGCUUUG | 1755 | CAAAGCCA GGCTAGCTACAACGA CCAAGGCA | 9669 |
| 1893 | CUIGGGUG G CUUUGGGG | 1756 | CCCCAAAG GGCTAGCTACAACGA CACCCAAG | 9670 |
| 1901 | GCUUUGGG G CAUGGACA | 1757 | TGTCCATG GGCTAGCTACAACGA CCCAAAGC | 9671 |
| 1917 | AUUGACCC G UAUAAAGA | 1758 | TCTTTATA GGCTAGCTACAACGA GGGTCAAT | 9672 |
| 1933 | AAUUUGGA G CUUCUGUG | 1759 | CACAGAAG GGCTAGCTACAACGA TCCAAATT | 9673 |
| 1944 | UCUGUGGA G UUACUCUC | 1760 | GAGAGTAA GGCTAGCTACAACGA TCCACAGA | 9674 |
| 2023 | AUCGGGGG G CCUUAGAG | 1761 | CTCTAAGG GGCTAGCTACAACGA CCCCCGAT | 9675 |
| 2031 | GCCUUAGA G UCUCCGGA | 1762 | TCCGGAGA GGCTAGCTACAACGA TCTAAGGC | 9676 |
| 2062 | ACCAUACG G CACUCAGG | 1763 | CCTGAGTG GGCTAGCTACAACGA CGTATGGT | 9677 |
| 2070 | GCACUCAG G CAAGCUAU | 1764 | ATAGCTTG GGCTAGCTACAACGA CTGAGTGC | 9678 |
| 2074 | UCAGGCAA G CUAUUCUG | 1765 | CAGAATAG GGCTAGCTACAACGA TTGCCTGA | 9679 |
| 2090 | GUGUUGGG G UGAGUUGA | 1766 | TCAACTCA GGCTAGCTACAACGA CCCAACAC | 9680 |
| 2094 | UGGGGUGA G UUGAUGAA | 1767 | TTCATCAA GGCTAGCTACAACGA TCACCCCA | 9681 |
| 2107 | UGAAUCUA G CCACCUGG | 1768 | CCAGGTGG GGCTAGCTACAACGA TAGATTCA | 9682 |
| 2116 | CCACCUGG G UGGGAAGU | 1769 | ACTTCCCA GGCTAGCTACAACGA CCAGGTGG | 9683 |
| 2123 | GGUGGGAA G UAAUUUGG | 1770 | CCAAATTA GGCTAGCTACAACGA TTCCCACC | 9684 |
| 2140 | AAGAUCCA G CAUCCAGG | 1771 | CCTGGATG GGCTAGCTACAACGA TGGATCTT | 9685 |
| 2155 | GGGAAUUA G UAGUCAGC | 1772 | GCTGACTA GGCTAGCTACAACGA TAATTCCC | 9686 |
| 2158 | AAUUAGUA G UCAGCUAU | 1773 | ATAGCTGA GGCTAGCTACAACGA TACTAATT | 9687 |
| 2162 | AGUAGUCA G CUAUGUCA | 1774 | TGACATAG GGCTAGCTACAACGA TGACTACT | 9688 |
| 2173 | AUGUCAAC G UUAAUAUG | 1775 | CATATTAA GGCTAGCTACAACGA GTTGACAT | 9689 |
| 2183 | UAAUAUGG G CCUAAAAA | 1776 | TTTTTAGG GGCTAGCTACAACGA CCATATTA | 9690 |
| 2208 | CUAUUGUG G UUUCACAU | 1777 | ATGTGAAA GGCTAGCTACAACGA CACAATAG | 9691 |
| 2235 | ACUUUUGG G CGAGAAAC | 1778 | GTTTCTCG GGCTAGCTACAACGA CCAAAAGT | 9692 |
| 2260 | AAUAUUUG G UGUCUUUU | 1779 | AAAAGACA GGCTAGCTACAACGA CAAATATT | 9693 |
| 2272 | CUUUUGGA G UGUGGAUU | 1780 | AATCCACA GGCTAGCTACAACGA TCCAAAAG | 9694 |
| 2360 | ACGAAGAG G CAGGUCCC | 1781 | GGGACCTG GGCTAGCTACAACGA CTCTTCGT | 9695 |
| 2364 | AGAGGCAG G UCCCCUAG | 1782 | CTAGGGGA GGCTAGCTACAACGA CTGCCTCT | 9696 |
| 2403 | AGACGAAG G UCUCAAUC | 1783 | GATTGAGA GGCTAGCTACAACGA CTTCGTCT | 9697 |
| 2417 | AUCGCCGC G UCGCAGAA | 1784 | TTCTGCGA GGCTAGCTACAACGA GCGGCGAT | 9698 |
| 2454 | CAAUGUUA G UAUUCCUU | 1785 | AAGGAATA GGCTAGCTACAACGA TAACATTG | 9699 |
| 2474 | CACAUAAG G UGGGAAAC | 1786 | GTTTCCCA GGCTAGCTACAACGA CTTATGTG | 9700 |
| 2491 | UUUACGGG G CUUUAUUC | 1787 | GAATAAAG GGCTAGCTACAACGA CCCGTAAA | 9701 |
| 2507 | CUUCUACG G UACCUUGC | 1788 | GCAAGGTA GGCTAGCTACAACGA CGTAGAAG | 9702 |
| 2530 | CCUAAAUG G CAAACUCC | 1789 | GGAGTTTG GGCTAGCTACAACGA CATTTAGG | 9703 |
| 2587 | AGAUGUAA G CAAUUUGU | 1790 | ACAAATTG GGCTAGCTACAACGA TTACATCT | 9704 |
| 2599 | UUUGUGGG G CCCCUUAC | 1791 | GTAAGGGG GGCTAGCTACAACGA CCCACAAA | 9705 |
| 2609 | CCCUUACA G UAAAUGAA | 1792 | TTCATTTA GGCTAGCTACAACGA TGTAAGGG | 9706 |
| 2650 | CCUGCUAG G UUUUAUCC | 1793 | GGATAAAA GGCTAGCTACAACGA CTAGCAGG | 9707 |
| 2701 | AUCAAACC G UAUUAUCC | 1794 | GGATAATA GGCTAGCTACAACGA GGTTTGAT | 9708 |
| 2713 | UAUCCAGA G UAUGUAGU | 1795 | ACTACATA GGCTAGCTACAACGA TCTGGATA | 9709 |
| 2720 | AGUAUGUA G UUAAUCAU | 1796 | ATGATTAA GGCTAGCTACAACGA TACATACT | 9710 |
| 2768 | UUUGGAAG G CGGGGAUC | 1797 | GATCCCCG GGCTAGCTACAACGA CTTCCAAA | 9711 |
| 2791 | AAAAGAGA G UCCACACG | 1798 | CGTGTGGA GGCTAGCTACAACGA TCTCTTTT | 9712 |
| 2799 | GUCCACAC G UAGCGCCU | 1799 | AGGCGCTA GGCTAGCTACAACGA GTGTGGAC | 9713 |
| 2802 | CACACGUA G CGCCUCAU | 1800 | ATGAGGCG GGCTAGCTACAACGA TACGTGTG | 9714 |
| 2818 | UUUUGCGG G UCACCAUA | 1801 | TATGGTGA GGCTAGCTACAACGA CCGCAAAA | 9715 |
| 2848 | GAUCUACA G CAUGGGAG | 1802 | CTCCCATG GGCTAGCTACAACGA TGTAGATC | 9716 |
| 2857 | CAUGGGAG G UUGGUCUU | 1803 | AAGACCAA GGCTAGCTACAACGA CTCCCATG | 9717 |
| 2861 | GGAGGUUG G UCUUCCAA | 1804 | TTGGAAGA GGCTAGCTACAACGA CAACCTCC | 9718 |
| 2881 | UCGAAAAG G CAUGGGGA | 1805 | TCCCCATG GGCTAGCTACAACGA CTTTTCGA | 9719 |
| 2936 | GAUCAUCA G UUGGACCC | 1806 | GGGTCCAA GGCTAGCTACAACGA TGATGATC | 9720 |
| 2955 | CAUUCAAA G CCAACUCA | 1807 | TGAGTTGG GGCTAGCTACAACGA TTTGAATG | 9721 |
| 2964 | CCAACUCA G UAAAUCCA | 1808 | TGGATTTA GGCTAGCTACAACGA TGAGTTGG | 9722 |
| 3005 | GACAACUG G CCGGACGC | 1809 | GCGTCCGG GGCTAGCTACAACGA CAGTTGTC | 9723 |
| 3021 | CCAACAAG G UGGGAGUG | 1810 | CACTCCCA GGCTAGCTACAACGA CTTGTTGG | 9724 |
| 3027 | AGGUGGGA G UGGGAGCA | 1811 | TGCTCCCA GGCTAGCTACAACGA TCCCACCT | 9725 |
| 3033 | GAGUGGGA G CAUUCGGG | 1812 | CCCGAATG GGCTAGCTACAACGA TCCCACTC | 9726 |
| 3041 | GCAUUCGG G CCAGGGUU | 1813 | AACCCTGG GGCTAGCTACAACGA CCGAATGC | 9727 |
| 3047 | GGGCCAGG G UUCACCCC | 1814 | GGGGTGAA GGCTAGCTACAACGA CCTGGCCC | 9728 |
| 3077 | CUGUUGGG G UGGAGCCC | 1815 | GGGCTCCA GGCTAGCTACAACGA CCCAACAG | 9729 |
| 3082 | GGGGUGGA G CCCUCACG | 1816 | CGTGAGGG GGCTAGCTACAACGA TCCACCCC | 9730 |
| 3097 | CGCUCAGG G CCUACUCA | 1817 | TGAGTAGG GGCTAGCTACAACGA CCTGAGCG | 9731 |
| 3117 | CUGUGCCA G CAGCUCCU | 1818 | AGGAGCTG GGCTAGCTACAACGA TGGCACAG | 9732 |
| 3120 | UGCCAGCA G CUCCUCCU | 1819 | AGGAGGAG GGCTAGCTACAACGA TGCTGGCA | 9733 |
| 3146 | ACCAAUCG G CAGUCAGG | 1820 | CCTGACTG GGCTAGCTACAACGA CGATTGGT | 9734 |
| 3149 | AAUCGGCA G UCAGGAAG | 1821 | CTTCCTGA GGCTAGCTACAACGA TGCCGATT | 9735 |
| 3158 | UCAGGAAG G CAGCCUAC | 1822 | GTAGGCTG GGCTAGCTACAACGA CTTCCTGA | 9736 |
| 3161 | GGAAGGCA G CCUACUCC | 1823 | GGAGTAGG GGCTAGCTACAACGA TGCCTTCC | 9737 |
| 3204 | AUCCUCAG G CCAUGCAG | 1824 | CTGCATGG GGCTAGCTACAACGA CTGAGGAT | 9738 |
| 10 | ACUCCACC A CUUUCCAC | 703 | GTGGAAAG GGCTAGCTACAACGA GGTGGAGT | 9739 |
| 17 | CACUUUCC A CCAAACUC | 706 | GAGTTTGG GGCTAGCTACAACGA GGAAAGTG | 9740 |
| 22 | UCCACCAA A CUCUUCAA | 1825 | TTGAAGAG GGCTAGCTACAACGA TTGGTGGA | 9741 |
| 32 | UCUUCAAG A UCCCAGAG | 1826 | CTCTGGGA GGCTAGCTACAACGA CTTGAAGA | 9742 |
| 53 | GGCCCUGU A CUUUCCUG | 42 | CAGGAAAG GGCTAGCTACAACGA ACAGGGCC | 9743 |
| 82 | GUUCAGGA A CAGUGAGC | 1827 | GCTCACTG GGCTAGCTACAACGA TCCTGAAC | 9744 |
| 101 | UGCUCAGA A UACUGUCU | 1828 | AGACAGTA GGCTAGCTACAACGA TCTGAGCA | 9745 |
| 103 | CUCAGAAU A CUGUCUCU | 50 | AGAGACAG GGCTAGCTACAACGA ATTCTGAG | 9746 |
| 115 | UCUCUGCC A UAUCGUCA | 733 | TGACGATA GGCTAGCTACAACGA GGCAGAGA | 9747 |
| 117 | UCUGCCAU A UCGUCAAU | 53 | ATTGACGA GGCTAGCTACAACGA ATGGCAGA | 9748 |
| 124 | UAUCGUCA A UCUUAUCG | 1829 | CGATAAGA GGCTAGCTACAACGA TGACGATA | 9749 |
| 129 | UCAAUCUU A UCGAAGAC | 58 | GTCTTCGA GGCTAGCTACAACGA AAGATTGA | 9750 |
| 136 | UAUCGAAG A CUGGGGAC | 1830 | GTCCCCAG GGCTAGCTACAACGA CTTCGATA | 9751 |
| 143 | GACUGGGG A CCCUGUAC | 1831 | GTACAGGG GGCTAGCTACAACGA CCCCAGTC | 9752 |
| 150 | CACCCUGU A CCGAACAU | 60 | ATGTTCGG GGCTAGCTACAACGA ACAGGGTC | 9753 |
| 155 | UGUACCGA A CAUGGAGA | 1832 | TCTCCATG GGCTAGCTACAACGA TCGGTACA | 9754 |
| 157 | UACCGAAC A UGGAGAAC | 745 | GTTCTCCA GGCTAGCTACAACGA GTTCGGTA | 9755 |
| 164 | CAUGGAGA A CAUCGCAU | 1833 | ATGCGATG GGCTAGCTACAACGA TCTCCATG | 9756 |
| 166 | UGGAGAAC A UCGCAUCA | 746 | TGATGCGA GGCTAGCTACAACGA GTTCTCCA | 9757 |
| 171 | AACAUCGC A UCAGGACU | 747 | AGTCCTGA GGCTAGCTACAACGA GCGATGTT | 9758 |
| 177 | GCAUCAGG A CUCCUAGG | 1834 | CCTAGGAG GGCTAGCTACAACGA CCTGATGC | 9759 |
| 186 | CUCCUAGG A CCCCUGCU | 1835 | AGCAGGGG GGCTAGCTACAACGA CCTAGGAG | 9760 |
| 201 | CUCGUGUU A CAGGCGGG | 67 | CCCGCCTG GGCTAGCTACAACGA AACACGAG | 9761 |
| 223 | UCUUGUUG A CAAAAAUC | 1836 | GATTTTTG GGCTAGCTACAACGA CAACAAGA | 9762 |
| 229 | UGACAAAA A UCCUCACA | 1837 | TGTGAGGA GGCTAGCTACAACGA TTTTGTCA | 9763 |
| 235 | AAAUCCUC A CAAUACCA | 762 | TGGTATTG GGCTAGCTACAACGA GAGGATTT | 9764 |
| 238 | UCCUCACA A UACCACAG | 1838 | CTGTGGTA GGCTAGCTACAACGA TGTGAGGA | 9765 |
| 240 | CUCACAAU A CCACAGAG | 77 | CTCTGTGG GGCTAGCTACAACGA ATTGTGAG | 9766 |
| 243 | ACAAUACC A CAGAGUCU | 765 | AGACTCTG GGCTAGCTACAACGA GGTATTGT | 9767 |
| 254 | GAGUCUAG A CUCGUGGU | 1839 | ACCACGAG GGCTAGCTACAACGA CTAGACTC | 9768 |
| 265 | CGUGGUGG A CUUCUCUC | 1840 | GAGAGAAG GGCTAGCTACAACGA CCACCACG | 9769 |
| 275 | UUCUCUCA A UUUUCUAG | 1841 | CTAGAAAA GGCTAGCTACAACGA TGAGAGAA | 9770 |
| 289 | UAGGGGGA A CACCCGUG | 1842 | CACGGGTG GGCTAGCTACAACGA TCCCCCTA | 9771 |
| 291 | GGGGGAAC A CCCGUGUG | 774 | CACACGGG GGCTAGCTACAACGA GTTCCCCC | 9772 |
| 311 | UGGCCAAA A UUCGCAGU | 1843 | ACTGCGAA GGCTAGCTACAACGA TTTGGCCA | 9773 |
| 325 | AGUCCCAA A UCUCCAGU | 1844 | ACTGGAGA GGCTAGCTACAACGA TTGGGACT | 9774 |
| 335 | CUCCAGUC A CUCACCAA | 787 | TTGGTGAG GGCTAGCTACAACGA GACTGGAG | 9775 |
| 339 | AGUCACUC A CCAACCUG | 789 | CAGGTTGG GGCTAGCTACAACGA GAGTGACT | 9776 |
| 343 | ACUCACCA A CCUGUUGU | 1845 | ACAACAGG GGCTAGCTACAACGA TGGTGAGT | 9777 |
| 358 | GUCCUCCA A UUUGUCCU | 1846 | AGGACAAA GGCTAGCTACAACGA TGGAGGAC | 9778 |
| 371 | UCCUGGUU A UCGCUGGA | 106 | TCCAGCGA GGCTAGCTACAACGA AACCAGGA | 9779 |
| 379 | AUCGCUGG A UGUGUCUG | 1847 | CAGACACA GGCTAGCTACAACGA CCAGCGAT | 9780 |
| 397 | GGCGUUUU A UCAUCUUC | 112 | GAAGATGA GGCTAGCTACAACGA AAAACGCC | 9781 |
| 400 | GUUUUAUC A UCUUCCUC | 802 | GAGGAAGA GGCTAGCTACAACGA GATAAAAC | 9782 |
| 412 | UCCUCUGC A UCCUGCUG | 807 | CAGCAGGA GGCTAGCTACAACGA GCAGAGGA | 9783 |
| 423 | CUGCUGCU A UGCCUCAU | 119 | ATGAGGCA GGCTAGCTACAACGA AGCAGCAG | 9784 |
| 430 | UAUGCCUC A UCUUCUUG | 814 | CAAGAAGA GGCTAGCTACAACGA GAGGCATA | 9785 |
| 452 | UCUUCUGG A CUAUCAAG | 1848 | CTTGATAG GGCTAGCTACAACGA CCAGAAGA | 9786 |
| 455 | UCUGGACU A UCAAGGUA | 130 | TACCTTGA GGCTAGCTACAACGA AGTCCAGA | 9787 |
| 463 | AUCAAGGU A UGUUGCCC | 132 | GGGCAACA GGCTAGCTACAACGA ACCTTGAT | 9788 |
| 484 | GUCCUCUA A UUCCAGGA | 1849 | TCCTGGAA GGCTAGCTACAACGA TAGAGGAC | 9789 |
| 492 | AUUCCAGG A UCAUCAAC | 1850 | GTTGATGA GGCTAGCTACAACGA CCTGGAAT | 9790 |
| 495 | CCAGGAUC A UCAACAAC | 828 | GTTGTTGA GGCTAGCTACAACGA GATCCTGG | 9791 |
| 499 | GAUCAUCA A CAACCAGC | 1851 | GCTGGTTG GGCTAGCTACAACGA TGATGATC | 9792 |
| 502 | CAUCAACA A CCAGCACC | 1852 | GGTGCTGG GGCTAGCTACAACGA TGTTGATG | 9793 |
| 513 | AGCACCGG A CCAUGCAA | 1853 | TTGCATGG GGCTAGCTACAACGA CCGGTGCT | 9794 |
| 516 | ACCGGACC A UGCAAAAC | 836 | GTTTTGCA GGCTAGCTACAACGA GGTCCGGT | 9795 |
| 523 | CAUGCAAA A CCUGCACA | 1854 | TGTGCAGG GGCTAGCTACAACGA TTTGCATG | 9796 |
| 529 | AAACCUGC A CAACUCCU | 840 | AGGAGTTG GGCTAGCTACAACGA GCAGGTTT | 9797 |
| 532 | CCUGCACA A CUCCUGCU | 1855 | AGCAGGAG GGCTAGCTACAACGA TGTGCAGG | 9798 |
| 547 | CUCAAGGA A CCUCUAUG | 1856 | CATAGAGG GGCTAGCTACAACGA TCCTTGAG | 9799 |
| 553 | GAACCUCU A UGUUUCCC | 146 | GGGAAACA GGCTAGCTACAACGA AGAGGTTC | 9800 |
| 564 | UUUCCCUC A UGUUGCUG | 853 | CAGCAACA GGCTAGCTACAACGA GAGGGAAA | 9801 |
| 574 | GUUGCUGU A CAAAACCU | 152 | AGGTTTTG GGCTAGCTACAACGA ACAGCAAC | 9802 |
| 579 | UGUACAAA A CCUACGGA | 1857 | TCCGTAGG GGCTAGCTACAACGA TTTGTACA | 9803 |
| 583 | CAAAACCU A CGGACGGA | 153 | TCCGTCCG GGCTAGCTACAACGA AGGTTTTG | 9804 |
| 587 | ACCUACGG A CGGAAACU | 1858 | AGTTTCCG GGCTAGCTACAACGA CCGTAGGT | 9805 |
| 593 | GGACGGAA A CUGCACCU | 1859 | AGGTGCAG GGCTAGCTACAACGA TTCCGTCC | 9806 |
| 598 | GAAACUGC A CCUGUAUU | 859 | AATACAGG GGCTAGCTACAACGA GCAGTTTC | 9807 |
| 604 | GCACCUGU A UUCCCAUC | 154 | GATGGGAA GGCTAGCTACAACGA ACAGGTGC | 9808 |
| 610 | GUAUUCCC A UCCCAUCA | 864 | TGATGGGA GGCTAGCTACAACGA GGGAATAC | 9809 |
| 615 | CCCAUCCC A UCAUCUUG | 867 | CAAGATGA GGCTAGCTACAACGA GGGATGGG | 9810 |
| 618 | AUCCCAUC A UCUUGGGC | 868 | GCCCAAGA GGCTAGCTACAACGA GATGGGAT | 9811 |
| 636 | UUCGCAAA A UACCUAUG | 1860 | CATAGGTA GGCTAGCTACAACGA TTTGCGAA | 9812 |
| 638 | CGCAAAAU A CCUAUGGG | 164 | CCCATAGG GGCTAGCTACAACGA ATTTTGCG | 9813 |
| 642 | AAAUACCU A UGGGAGUG | 165 | CACTCCCA GGCTAGCTACAACGA AGGTATTT | 9814 |
| 681 | CUCAGUUU A CUAGUGCC | 176 | GGCACTAG GGCTAGCTACAACGA AAACTGAG | 9815 |
| 690 | CUAGUGCC A UUUGUUCA | 884 | TGAACAAA GGCTAGCTACAACGA GGCACTAG | 9816 |
| 721 | UUUCCCCC A CUGUCUGG | 891 | CCAGACAG GGCTAGCTACAACGA GGGGGAAA | 9817 |
| 739 | UUUCAGUU A UAUGGAUG | 193 | CATCCATA GGCTAGCTACAACGA AACTGAAA | 9818 |
| 741 | UCAGUUAU A UGGAUGAU | 194 | ATCATCCA GGCTAGCTACAACGA ATAACTGA | 9819 |
| 745 | UUAUAUGG A UGAUGUGG | 1861 | CCACATCA GGCTAGCTACAACGA CCATATAA | 9820 |
| 748 | UAUGGAUG A UGUGGUUU | 1862 | AAACCACA GGCTAGCTACAACGA CATCCATA | 9821 |
| 773 | AAGUCUGU A CAACAUCU | 199 | AGATGTTG GGCTAGCTACAACGA ACAGACTT | 9822 |
| 776 | UCUGUACA A CAUCUUGA | 1863 | TCAAGATG GGCTAGCTACAACGA TGTACAGA | 9823 |
| 778 | UGUACAAC A UCUUGAGU | 900 | ACTCAAGA GGCTAGCTACAACGA GTTGTACA | 9824 |
| 793 | GUCCCUUU A UGCCGCUG | 205 | CAGCGGCA GGCTAGCTACAACGA AAAGGGAC | 9825 |
| 804 | CCGCUGUU A CCAAUUUU | 207 | AAAATTGG GGCTAGCTACAACGA AACAGCGG | 9826 |
| 808 | UGUUACCA A UUUUCUUU | 1864 | AAAGAAAA GGCTAGCTACAACGA TGGTAACA | 9827 |
| 828 | CUUUGGGU A UACAUUUA | 218 | TAAATGTA GGCTAGCTACAACGA ACCCAAAG | 9828 |
| 830 | UUGGGUAU A CAUUUAAA | 219 | TTTAAATG GGCTAGCTACAACGA ATACCCAA | 9829 |
| 832 | GGGUAUAC A UUUAAACC | 911 | GGTTTAAA GGCTAGCTACAACGA GTATACCC | 9830 |
| 838 | ACAUUUAA A CCCUCACA | 1865 | TGTGAGGG GGCTAGCTACAACGA TTAAATGT | 9831 |
| 844 | AAACCCUC A CAAAACAA | 915 | TTGTTTTG GGCTAGCTACAACGA GAGGGTTT | 9832 |
| 849 | CUCACAAA A CAAAAAGA | 1866 | TCTTTTTG GGCTAGCTACAACGA TTTGTGAG | 9833 |
| 857 | ACAAAAAG A UGGGGAUA | 1867 | TATCCCCA GGCTAGCTACAACGA CTTTTTGT | 9834 |
| 863 | AGAUGGGG A UAUUCCCU | 1868 | AGGGAATA GGCTAGCTACAACGA CCCCATCT | 9835 |
| 865 | AUGGGGAU A UUCCCUUA | 224 | TAAGGGAA GGCTAGCTACAACGA ATCCCCAT | 9836 |
| 874 | UUCCCUUA A CUUCAUGG | 1869 | CCATGAAG GGCTAGCTACAACGA TAAGGGAA | 9837 |
| 879 | UUAACUUC A UGGGAUAU | 922 | ATATCCCA GGCTAGCTACAACGA GAAGTTAA | 9838 |
| 884 | UUCAUGGG A UAUGUAAU | 1870 | ATTACATA GGCTAGCTACAACGA CCCATGAA | 9839 |
| 886 | CAUGGGAU A UGUAAUUG | 231 | CAATTACA GGCTAGCTACAACGA ATCCCATG | 9840 |
| 891 | GAUAUGUA A UUGGGAGU | 1871 | ACTCCCAA GGCTAGCTACAACGA TACATATC | 9841 |
| 906 | GUUGGGGC A CAUUGCCA | 923 | TGGCAATG GGCTAGCTACAACGA GCCCCAAC | 9842 |
| 908 | UGGGGCAC A UUGCCACA | 924 | TGTGGCAA GGCTAGCTACAACGA GTGCCCCA | 9843 |
| 914 | ACAUUGCC A CAGGAACA | 926 | TGTTCCTG GGCTAGCTACAACGA GGCAATGT | 9844 |
| 920 | CCACAGGA A CAUAUUGU | 1872 | ACAATATG GGCTAGCTACAACGA TCCTGTGG | 9845 |
| 922 | ACAGGAAC A UAUUGUAC | 928 | GTACAATA GGCTAGCTACAACGA GTTCCTGT | 9846 |
| 924 | AGGAACAU A UUGUACAA | 236 | TTGTACAA GGCTAGCTACAACGA ATGTTCCT | 9847 |
| 929 | CAUAUUGU A CAAAAAAU | 238 | ATTTTTTG GGCTAGCTACAACGA ACAATATG | 9848 |
| 936 | UACAAAAA A UCAAAAUG | 1873 | CATTTTGA GGCTAGCTACAACGA TTTTTGTA | 9849 |
| 942 | AAAUCAAA A UGUGUUUU | 1874 | AAAACACA GGCTAGCTACAACGA TTTGATTT | 9850 |
| 956 | UUUAGGAA A CUUCCUGU | 1875 | ACAGGAAG GGCTAGCTACAACGA TTCCTAAA | 9851 |
| 967 | UCCUGUAA A CAGGCCUA | 1876 | TAGGCCTG GGCTAGCTACAACGA TTACAGGA | 9852 |
| 975 | ACAGGCCU A UUGAUUGG | 247 | CCAATCAA GGCTAGCTACAACGA AGGCCTGT | 9853 |
| 979 | GCCUAUUG A UUGGAAAG | 1877 | CTTTCCAA GGCTAGCTACAACGA CAATAGGC | 9854 |
| 989 | UGGAAAGU A UGUCAACG | 250 | CGTTGACA GGCTAGCTACAACGA ACTTTCCA | 9855 |
| 995 | GUAUGUCA A CGAAUUGU | 1878 | ACAATTCG GGCTAGCTACAACGA TGACATAC | 9856 |
| 999 | GUCAACGA A UUGUGGGU | 1879 | ACCCACAA GGCTAGCTACAACGA TCGTTGAC | 9857 |
| 1032 | CCCCUUUC A CGCAAUGU | 944 | ACATTGCG GGCTAGCTACAACGA GAAAGGGG | 9858 |
| 1037 | UUCACGCA A UGUGGAUA | 1880 | TATCCACA GGCTAGCTACAACGA TGCGTGAA | 9859 |
| 1043 | CAAUGUGG A UAUUCUGC | 1881 | GCAGAATA GGCTAGCTACAACGA CCACATTG | 9860 |
| 1045 | AUGUGGAU A UUCUGCUU | 262 | AAGCAGAA GGCTAGCTACAACGA ATCCACAT | 9861 |
| 1056 | CUGCUUUA A UGCCUUUA | 1882 | TAAAGGCA GGCTAGCTACAACGA TAAAGCAG | 9862 |
| 1064 | AUGCCUUU A UAUGCAUG | 270 | CATGCATA GGCTAGCTACAACGA AAAGGCAT | 9863 |
| 1066 | GCCUUUAU A UGCAUGCA | 271 | TGCATGCA GGCTAGCTACAACGA ATAAAGGC | 9864 |
| 1070 | UUAUAUGC A UGCAUACA | 950 | TGTATGCA GGCTAGCTACAACGA GCATATAA | 9865 |
| 1074 | AUGCAUGC A UACAAGCA | 951 | TGCTTGTA GGCTAGCTACAACGA GCATGCAT | 9866 |
| 1076 | GCAUGCAU A CAAGCAAA | 272 | TTTGCTTG GGCTAGCTACAACGA ATGCATGC | 9867 |
| 1085 | CAAGCAAA A CAGGCUUU | 1883 | AAAGCCTG GGCTAGCTACAACGA TTTGCTTG | 9868 |
| 1095 | AGGCUUUU A CUUUCUCG | 276 | CGAGAAAG GGCTAGCTACAACGA AAAAGCCT | 9869 |
| 1107 | UCUCGCCA A CUUACAAG | 1884 | CTTGTAAG GGCTAGCTACAACGA TGGCGAGA | 9870 |
| 1111 | GCCAACUU A CAAGGCCU | 282 | AGGCCTTG GGCTAGCTACAACGA AAGTTGGC | 9871 |
| 1130 | CUAAGUAA A CAGUAUGU | 1885 | ACATACTG GGCTAGCTACAACGA TTACTTAG | 9872 |
| 1135 | UAAACAGU A UGUGAACC | 288 | GGTTCACA GGCTAGCTACAACGA ACTGTTTA | 9873 |
| 1141 | GUAUGUGA A CCUUUACC | 1886 | GGTAAAGG GGCTAGCTACAACGA TCACATAC | 9874 |
| 1147 | GAACCUUU A CCCCGUUG | 291 | CAACGGGG GGCTAGCTACAACGA AAAGGTTC | 9875 |
| 1163 | GCUCGGCA A CGGCCUGG | 1887 | CCAGGCCG GGCTAGCTACAACGA TGCCGAGC | 9876 |
| 1175 | CCUGGUCU A UGCCAAGU | 295 | ACTTGGCA GGCTAGCTACAACGA AGACCAGG | 9877 |
| 1192 | GUUUGCUG A CGCAACCC | 1888 | GGGTTGCG GGCTAGCTACAACGA CAGCAAAC | 9878 |
| 1197 | CUGACGCA A CCCCCACU | 1889 | AGTGGGGG GGCTAGCTACAACGA TGCGTCAG | 9879 |
| 1203 | CAACCCCC A CUGGUUGG | 984 | CCAACCAG GGCTAGCTACAACGA GGGGGTTG | 9880 |
| 1221 | GCUUGGCC A UAGGCCAU | 988 | ATGGCCTA GGCTAGCTACAACGA GGCCAAGC | 9881 |
| 1228 | CAUAGGCC A UCAGCGCA | 990 | TGCGCTGA GGCTAGCTACAACGA GGCCTATG | 9882 |
| 1236 | AUCAGCGC A UGCGUGGA | 992 | TCCACGCA GGCTAGCTACAACGA GCGCTGAT | 9883 |
| 1245 | UGCGUGGA A CCUUUGUG | 1890 | CACAAAGG GGCTAGCTACAACGA TCCACGCA | 9884 |
| 1266 | CUCUGCCG A UCCAUACC | 1891 | GGTATGGA GGCTAGCTACAACGA CGGCAGAG | 9885 |
| 1270 | GCCGAUCC A UACCGCGG | 1001 | CCGCGGTA GGCTAGCTACAACGA GGATCGGC | 9886 |
| 1272 | CGAUCCAU A CCGCGGAA | 308 | TTCCGCGG GGCTAGCTACAACGA ATGGATCG | 9887 |
| 1280 | ACCGCGGA A CUCCUAGC | 1892 | GCTAGGAG GGCTAGCTACAACGA TCCGCGGT | 9888 |
| 1322 | GGGGCAAA A CUCAUCGG | 1893 | CCGATGAG GGCTAGCTACAACGA TTTGCCCC | 9889 |
| 1326 | CAAAACUC A UCGGGACU | 1014 | AGTCCCGA GGCTAGCTACAACGA GAGTTTTG | 9890 |
| 1332 | UCAUCGGG A CUGACAAU | 1894 | ATTGTCAG GGCTAGCTACAACGA CCCGATGA | 9891 |
| 1336 | CGGGACUG A CAAUUCUG | 1895 | CAGAATTG GGCTAGCTACAACGA CAGTCCCG | 9892 |
| 1339 | GACUGACA A UUCUGUCG | 1896 | CGACAGAA GGCTAGCTACAACGA TGTCAGTC | 9893 |
| 1361 | UCCCGCAA A UAUACAUC | 1897 | GATGTATA GGCTAGCTACAACGA TTGCGGGA | 9894 |
| 1363 | CCGCAAAU A UACAUCAU | 324 | ATGATGTA GGCTAGCTACAACGA ATTTGCGG | 9895 |
| 1365 | GCAAAUAU A CAUCAUUU | 325 | AAATGATG GGCTAGCTACAACGA ATATTTGC | 9896 |
| 1367 | AAAUAUAC A UCAUUUCC | 1023 | GGAAATGA GGCTAGCTACAACGA GTATATTT | 9897 |
| 1370 | UAUACAUC A UUUCCAUG | 1024 | CATGGAAA GGCTAGCTACAACGA GATGTATA | 9898 |
| 1376 | UCAUUUCC A UGGCUGCU | 1026 | AGCAGCCA GGCTAGCTACAACGA GGAAATGA | 9899 |
| 1399 | UGCUGCCA A CUGGAUCC | 1898 | GGATCCAG GGCTAGCTACAACGA TGGCAGCA | 9900 |
| 1404 | CCAACUGG A UCCUACGC | 1899 | GCGTAGGA GGCTAGCTACAACGA CCAGTTGG | 9901 |
| 1409 | UGGAUCCU A CGCGGGAC | 332 | GTCCCGCG GGCTAGCTACAACGA AGGATCCA | 9902 |
| 1416 | UACGCGGG A CGUCCUUU | 1900 | AAAGGACG GGCTAGCTACAACGA CCCGCGTA | 9903 |
| 1429 | CUUUGUUU A CGUCCCGU | 338 | ACGGGACG GGCTAGCTACAACGA AAACAAAG | 9904 |
| 1447 | GGCGCUGA A UCCCGCGG | 1901 | CCGCGGGA GGCTAGCTACAACGA TCAGCGCC | 9905 |
| 1456 | UCCCGCGG A CGACCCCU | 1902 | AGGGGTCG GGCTAGCTACAACGA CCGCGGGA | 9906 |
| 1459 | CGCGGACG A CCCCUCCC | 1903 | GGGAGGGG GGCTAGCTACAACGA CGTCCGCG | 9907 |
| 1486 | GGGGCUCU A CCGCCCGC | 345 | GCGGGCGG GGCTAGCTACAACGA AGAGCCCC | 9908 |
| 1505 | CUCCGCCU A UUGUACCG | 349 | CGGTACAA GGCTAGCTACAACGA AGGCGGAG | 9909 |
| 1510 | CCUAUUGU A CCGACCGU | 351 | ACGGTCGG GGCTAGCTACAACGA ACAATAGG | 9910 |
| 1514 | UUGUACCG A CCGUCCAC | 1904 | GTGGACGG GGCTAGCTACAACGA CGGTACAA | 9911 |
| 1521 | GACCGUCC A CGGGGCGC | 1064 | GCGCCCCG GGCTAGCTACAACGA GGACGGTC | 9912 |
| 1530 | CGGGGCGC A CCUCUCUU | 1065 | AAGAGAGG GGCTAGCTACAACGA GCGCCCCG | 9913 |
| 1540 | CUCUCUUU A CGCGGACU | 357 | AGTCCGCG GGCTAGCTACAACGA AAAGAGAG | 9914 |
| 1546 | UUACGCGG A CUCCCCGU | 1905 | ACGGGGAG GGCTAGCTACAACGA CCGCGTAA | 9915 |
| 1567 | GCCUUCUC A UCUGCCGG | 1078 | CCGGCAGA GGCTAGCTACAACGA GAGAAGGC | 9916 |
| 1576 | UCUGCCGG A CCGUGUGC | 1906 | GCACACGG GGCTAGCTACAACGA CCGGCAGA | 9917 |
| 1585 | CCGUGUGC A CUUCGCUU | 1082 | AAGCGAAG GGCTAGCTACAACGA GCACACGG | 9918 |
| 1595 | UUCGCUUC A CCUCUGCA | 1085 | TGCAGAGG GGCTAGCTACAACGA GAAGCGAA | 9919 |
| 1603 | ACCUCUGC A CGUCGCAU | 1089 | ATGCGACG GGCTACCTACAACGA GCAGAGGT | 9920 |
| 1610 | CACGUCGC A UGGAGACC | 1090 | GGTCTCCA GGCTAGCTACAACGA GCGACGTG | 9921 |
| 1616 | GCAUGGAG A CCACCGUG | 1907 | CACGGTGG GGCTAGCTACAACGA CTCCATGC | 9922 |
| 1619 | UGGAGACC A CCGUGAAC | 1092 | GTTCACGG GGCTAGCTACAACGA GGTCTCCA | 9923 |
| 1626 | CACCGUGA A CGCCCACA | 1908 | TGTGGGCG GGCTAGCTACAACGA TCACGGTG | 9924 |
| 1638 | CCACAGGA A CCUGCCCA | 1909 | TGGGCAGG GGCTAGCTACAACGA TCCTGTGG | 9925 |
| 1656 | GGUCUUGC A UAAGAGGA | 1104 | TCCTCTTA GGCTAGCTACAACGA GCAAGACC | 9926 |
| 1664 | AUAAGAGG A CUCUUGGA | 1910 | TCCAAGAG GGCTAGCTACAACGA CCTCTTAT | 9927 |
| 1672 | ACUCUUGG A CUUUCAGC | 1911 | GCTGAAAG GGCTAGCTACAACGA CCAAGAGT | 9928 |
| 1682 | UUUCAGCA A UGUCAACG | 1912 | CGTTGACA GGCTAGCTACAACGA TGCTGAAA | 9929 |
| 1688 | CAAUGUCA A CGACCGAC | 1913 | GTCGGTCG GGCTAGCTACAACGA TGACATTG | 9930 |
| 1691 | UGUCAACG A CCGACCUU | 1914 | AAGGTCGG GGCTAGCTACAACGA CGTTGACA | 9931 |
| 1695 | AACGACCG A CCUUGAGG | 1915 | CCTCAAGG GGCTAGCTACAACGA CGGTCGTT | 9932 |
| 1705 | CUUGAGGC A UACUUCAA | 1114 | TTGAAGTA GGCTAGCTACAACGA GCCTCAAG | 9933 |
| 1707 | UGAGGCAU A CUUCAAAG | 380 | CTTTGAAG GGCTAGCTACAACGA ATGCCTCA | 9934 |
| 1716 | CUUCAAAG A CUGUGUGU | 1916 | ACACACAG GGCTAGCTACAACGA CTTTGAAG | 9935 |
| 1728 | UGUGUUUA A UGAGUGGG | 1917 | CCCACTCA GGCTAGCTACAACGA TAAACACA | 9936 |
| 1774 | GUCUUUGU A CUAGGAGG | 394 | CCTCCTAG GGCTAGCTACAACGA ACAAAGAC | 9937 |
| 1791 | CUGUAGGC A UAAAUUGG | 1121 | CCAATTTA GGCTAGCTACAACGA GCCTACAG | 9938 |
| 1795 | AGGCAUAA A UUGGUGUG | 1918 | CACACCAA GGCTAGCTACAACGA TTATGCCT | 9939 |
| 1807 | GUGUGUUC A CCAGCACC | 1122 | GGTGCTGG GGCTAGCTACAACGA GAACACAC | 9940 |
| 1813 | UCACCAGC A CCAUGCAA | 1125 | TTGCATGG GGCTAGCTACAACGA GCTGGTGA | 9941 |
| 1816 | CCAGCACC A UGCAACUU | 1127 | AAGTTGCA GGCTAGCTACAACGA GGTGCTGG | 9942 |
| 1821 | ACCAUGCA A CUUUUUCA | 1919 | TGAAAAAG GGCTAGCTACAACGA TGCATGGT | 9943 |
| 1829 | ACUUUUUC A CCUCUGCC | 1130 | GGCAGAGG GGCTAGCTACAACGA GAAAAAGT | 9944 |
| 1840 | UCUGCCUA A UCAUCUCA | 1920 | TGAGATGA GGCTAGCTACAACGA TAGGCAGA | 9945 |
| 1843 | GCCUAAUC A UCUCAUGU | 1136 | ACATGAGA GGCTAGCTACAACGA GATTAGGC | 9946 |
| 1848 | AUCAUCUC A UGUUCAUG | 1138 | CATGAACA GGCTAGCTACAACGA GAGATGAT | 9947 |
| 1854 | UCAUGUUC A UGUCCUAC | 1139 | GTAGGACA GGCTAGCTACAACGA GAACATGA | 9948 |
| 1861 | CAUGUCCU A CUGUUCAA | 414 | TTGAACAG GGCTAGCTACAACGA AGGACATG | 9949 |
| 1903 | UUUGGGGC A UGGACAUU | 1152 | AATGTCCA GGCTAGCTACAACGA GCCCCAAA | 9950 |
| 1907 | GGGCAUGG A CAUUGACC | 1921 | GGTCAATG GGCTAGCTACAACGA CCATGCCC | 9951 |
| 1909 | GCAUGGAC A UUGACCCG | 1153 | CGGGTCAA GGCTAGCTACAACGA GTCCATGC | 9952 |
| 1913 | GGACAUUG A CCCGUAUA | 1922 | TATACGGG GGCTAGCTACAACGA CAATGTCC | 9953 |
| 1919 | UGACCCGU A UAAAGAAU | 422 | ATTCTTTA GGCTAGCTACAACGA ACGGGTCA | 9954 |
| 1926 | UAUAAAGA A UUUGGAGC | 1923 | GCTCCAAA GGCTAGCTACAACGA TCTTTATA | 9955 |
| 1947 | GUGGAGUU A CUCUCUUU | 429 | AAAGAGAG GGCTAGCTACAACGA AACTCCAC | 9956 |
| 1967 | GCCUUCUG A CUUCUUUC | 1924 | GAAAGAAG GGCTAGCTACAACGA CAGAAGGC | 9957 |
| 1981 | UUCCUUCU A UUCGAGAU | 446 | ATCTCGAA GGCTAGCTACAACGA AGAAGGAA | 9958 |
| 1988 | UAUUCGAG A UCUCCUCG | 1925 | CGAGGAGA GGCTAGCTACAACGA CTCGAATA | 9959 |
| 1997 | UCUCCUCG A CACCGCCU | 1926 | AGGCGGTG GGCTAGCTACAACGA CGAGGAGA | 9960 |
| 1999 | UCCUCGAC A CCGCCUCU | 1172 | AGAGGCGG GGCTAGCTACAACGA GTCGAGGA | 9961 |
| 2015 | UGCUCUGU A UCGGGGGG | 454 | CCCCCCGA GGCTAGCTACAACGA ACAGAGCA | 9962 |
| 2040 | UCUCCGGA A CAUUGUUC | 1927 | GAACAATG GGCTAGCTACAACGA TCCGGAGA | 9963 |
| 2042 | UCCGGAAC A UUGUUCAC | 1183 | GTGAACAA GGCTAGCTACAACGA GTTCCGGA | 9964 |
| 2049 | CAUUGUUC A CCUCACCA | 1184 | TGGTGAGG GGCTAGCTACAACGA GAACAATG | 9965 |
| 2054 | UUCACCUC A CCAUACGG | 1187 | CCGTATGG GGCTAGCTACAACGA GAGGTGAA | 9966 |
| 2057 | ACCUCACC A UACGGCAC | 1189 | GTGCCGTA GGCTAGCTACAACGA GGTGAGGT | 9967 |
| 2059 | CUCACCAU A CGGCACUC | 464 | GAGTGCCG GGCTAGCTACAACGA ATGGTGAG | 9968 |
| 2064 | CAUACGGC A CUCAGGCA | 1190 | TGCCTGAC GGCTAGCTACAACGA GCCGTATG | 9969 |
| 2077 | GGCAAGCU A UUCUGUGU | 466 | ACACAGAA GGCTAGCTACAACGA AGCTTGCC | 9970 |
| 2098 | GUGAGUUG A UGAAUCUA | 1928 | TAGATTCA GGCTAGCTACAACGA CAACTCAC | 9971 |
| 2102 | GUUGAUGA A UCUAGCCA | 1929 | TGGCTAGA GGCTAGCTACAACGA TCATCAAC | 9972 |
| 2110 | AUCUAGCC A CCUGGGUG | 1198 | CACCCAGG GGCTAGCTACAACGA GGCTAGAT | 9973 |
| 2126 | GGGAAGUA A UUUGGAAG | 1930 | CTTCCAAA GGCTAGCTACAACGA TACTTCCC | 9974 |
| 2135 | UUUGGAAG A UCCAGCAU | 1931 | ATGCTGGA GGCTAGCTACAACGA CTTCCAAA | 9975 |
| 2142 | GAUCCAGC A UCCAGGGA | 1203 | TCCCTGGA GGCTAGCTACAACGA GCTGGATC | 9976 |
| 2151 | UCCAGGGA A UUAGUAGU | 1932 | ACTACTAA GGCTAGCTACAACGA TCCCTGGA | 9977 |
| 2165 | AGUCAGCU A UGUCAACG | 482 | CGTTGACA GGCTAGCTACAACGA AGCTGACT | 9978 |
| 2171 | CUAUGUCA A CGUUAAUA | 1933 | TATTAACG GGCTAGCTACAACGA TGACATAG | 9979 |
| 2177 | CAACGUUA A UAUGGGCC | 1934 | GGCCCATA GGCTAGCTACAACGA TAACGTTG | 9980 |
| 2179 | ACGUUAAU A UGGGCCUA | 486 | TAGGCCCA GGCTAGCTACAACGA ATTAACGT | 9981 |
| 2191 | GCCUAAAA A UCAGACAA | 1935 | TTGTCTGA GGCTAGCTACAACGA TTTTAGGC | 9982 |
| 2196 | AAAAUCAG A CAACUAUU | 1936 | AATAGTTG GGCTAGCTACAACGA CTGATTTT | 9983 |
| 2199 | AUCAGACA A CUAUUGUG | 1937 | CACAATAG GGCTAGCTACAACGA TGTCTGAT | 9984 |
| 2202 | AGACAACU A UUGUGGUU | 489 | AACCACAA GGCTAGCTACAACGA AGTTGTCT | 9985 |
| 2213 | GUGGUUUC A CAUUUCCU | 1214 | AGGAAATG GGCTAGCTACAACGA GAAACCAC | 9986 |
| 2215 | GGUUUCAC A UUUCCUGU | 1215 | ACAGGAAA GGCTAGCTACAACGA GTGAAACC | 9987 |
| 2227 | CCUGUCUU A CUUUUGGG | 499 | CCCAAAAG GGCTAGCTACAACGA AAGACAGG | 9988 |
| 2242 | GGCGAGAA A CUGUUCUU | 1938 | AAGAACAG GGCTAGCTACAACGA TTCTCGCC | 9989 |
| 2253 | GUUCUUGA A UAUUUGGU | 1939 | ACCAAATA GGCTAGCTACAACGA TCAAGAAC | 9990 |
| 2255 | UCUUGAAU A UUUGGUGU | 506 | ACACCAAA GGCTAGCTACAACGA ATTCAAGA | 9991 |
| 2278 | GAGUGUGG A UUCGCACU | 1940 | AGTGCGAA GGCTAGCTACAACGA CCACACTC | 9992 |
| 2284 | GGAUUCGC A CUCCUCCU | 1223 | AGGAGGAG GGCTAGCTACAACGA GCGAATCC | 9993 |
| 2295 | CCUCCUGC A UAUAGACC | 1229 | GGTCTATA GGCTAGCTACAACGA GCAGGAGG | 9994 |
| 2297 | UCCUGCAU A UAGACCAC | 517 | GTGGTCTA GGCTAGCTACAACGA ATGCAGGA | 9995 |
| 2301 | GCAUAUAG A CCACCAAA | 1941 | TTTGGTGG GGCTAGCTACAACGA CTATATGC | 9996 |
| 2304 | UAUAGACC A CCAAAUGC | 1231 | GCATTTGG GGCTAGCTACAACGA GGTCTATA | 9997 |
| 2309 | ACCACCAA A UGCCCCUA | 1942 | TAGGGGCA GGCTAGCTACAACGA TTGGTGGT | 9998 |
| 2317 | AUGCCCCU A UCUUAUCA | 519 | TGATAAGA GGCTAGCTACAACGA AGGGGCAT | 9999 |
| 2322 | CCUAUCUU A UCAACACU | 522 | AGTGTTGA GGCTAGCTACAACGA AAGATAGG | 10000 |
| 2326 | UCUUAUCA A CACUUCCG | 1943 | CGGAAGTG GGCTAGCTACAACGA TGATAAGA | 10001 |
| 2328 | UUAUCAAC A CUUCCGGA | 1240 | TCCGGAAG GGCTAGCTACAACGA GTTGATAA | 10002 |
| 2338 | UUCCGGAA A CUACUGUU | 1944 | AACAGTAG GGCTAGCTACAACGA TTCCGGAA | 10003 |
| 2341 | CGGAAACU A CUGUUGUU | 526 | AACAACAG GGCTAGCTACAACGA AGTTTCCG | 10004 |
| 2352 | GUUGUUAG A CGAAGAGG | 1945 | CCTCTTCG GGCTAGCTACAACGA CTAACAAC | 10005 |
| 2380 | GAAGAAGA A CUCCCUCG | 1946 | CGAGGGAG GGCTAGCTACAACGA TCTTCTTC | 10006 |
| 2397 | CCUCGCAG A CGAAGGUC | 1947 | GACCTTCG GGCTAGCTACAACGA CTGCGAGG | 10007 |
| 2409 | AGGUCUCA A UCGCCGCG | 1948 | CGCGGCGA GGCTAGCTACAACGA TGAGACCT | 10008 |
| 2427 | CGCAGAAG A UCUCAAUC | 1949 | GATTGAGA GGCTAGCTACAACGA CTTCTGCG | 10009 |
| 2433 | AGAUCUCA A UCUCGGGA | 1950 | TCCCGAGA GGCTAGCTACAACGA TGAGATCT | 10010 |
| 2442 | UCUCGGGA A UCUCAAUG | 1951 | CATTGAGA GGCTAGCTACAACGA TCCCGAGA | 10011 |
| 2448 | GAAUCUCA A UGUUAGUA | 1952 | TACTAACA GGCTAGCTACAACGA TGAGATTC | 10012 |
| 2456 | AUGUUAGU A UUCCUUGG | 547 | CCAAGGAA GGCTAGCTACAACGA ACTAACAT | 10013 |
| 2465 | UUCCUUGG A CACAUAAG | 1953 | CTTATGTG GGCTAGCTACAACGA CCAAGGAA | 10014 |
| 2467 | CCUUGGAC A CAUAAGGU | 1268 | ACCTTATG GGCTAGCTACAACGA GTCCAAGG | 10015 |
| 2469 | UUGGACAC A UAAGGUGG | 1269 | CCACCTTA GGCTAGCTACAACGA GTGTCCAA | 10016 |
| 2481 | GGUGGGAA A CUUUACGG | 1954 | CCGTAAAG GGCTAGCTACAACGA TTCCCACC | 10017 |
| 2486 | GAAACUUU A CGGGGCUU | 554 | AAGCCCCG GGCTAGCTACAACGA AAAGTTTC | 10018 |
| 2496 | GGGGCUUU A UUCUUCUA | 557 | TAGAAGAA GGCTAGCTACAACGA AAAGCCCC | 10019 |
| 2504 | AUUCUUCU A CGGUACCU | 562 | AGGTACCG GGCTAGCTACAACGA AGAAGAAT | 10020 |
| 2509 | UCUACGGU A CCUUGCUU | 563 | AAGCAAGG GGCTAGCTACAACGA ACCGTAGA | 10021 |
| 2520 | UUGCUUUA A UCCUAAAU | 1955 | ATTTAGGA GGCTAGCTACAACGA TAAAGCAA | 10022 |
| 2527 | AAUCCUAA A UGGCAAAC | 1956 | GTTTGCCA GGCTAGCTACAACGA TTAGGATT | 10023 |
| 2534 | AAUGGCAA A CUCCUUCU | 1957 | AGAAGGAG GGCTAGCTACAACGA TTGCCATT | 10024 |
| 2550 | UUUUCCUG A CAUUCAUU | 1958 | AATGAATG GGCTAGCTACAACGA CAGGAAAA | 10025 |
| 2552 | UUCCUGAC A UUCAUUUG | 1286 | CAAATGAA GGCTAGCTACAACGA GTCAGGAA | 10026 |
| 2556 | UGACAUUC A UUUGCAGG | 1287 | CCTGCAAA GGCTAGCTACAACGA GAATGTCA | 10027 |
| 2568 | GCAGGAGG A CAUUGUUG | 1959 | CAACAATG GGCTAGCTACAACGA CCTCCTGC | 10028 |
| 2570 | AGGAGGAC A UUGUUGAU | 1289 | ATCAACAA GGCTAGCTACAACGA GTCCTCCT | 10029 |
| 2577 | CAUUGUUG A UAGAUGUA | 1960 | TACATCTA GGCTAGCTACAACGA CAACAATG | 10030 |
| 2581 | GUUGAUAG A UGUAAGCA | 1961 | TGCTTACA GGCTAGCTACAACGA CTATCAAC | 10031 |
| 2590 | UGUAAGCA A UUUGUGGG | 1962 | CCCACAAA GGCTAGCTACAACGA TGCTTACA | 10032 |
| 2606 | GGCCCCUU A CAGUAAAU | 588 | ATTTACTG GGCTAGCTACAACGA AAGGGGCC | 10033 |
| 2613 | UACAGUAA A UGAAAACA | 1963 | TGTTTTCA GGCTAGCTACAACGA TTACTGTA | 10034 |
| 2619 | AAAUGAAA A CAGGAGAC | 1964 | GTCTCCTG GGCTAGCTACAACGA TTTCATTT | 10035 |
| 2626 | AACAGGAG A CUUAAAUU | 1965 | AATTTAAG GGCTAGCTACAACGA CTCCTGTT | 10036 |
| 2632 | AGACUUAA A UUAACUAU | 1966 | ATAGTTAA GGCTAGCTACAACGA TTAAGTCT | 10037 |
| 2636 | UUAAAUUA A CUAUGCCU | 1967 | AGGCATAG GGCTAGCTACAACGA TAATTTAA | 10038 |
| 2639 | AAUUAACU A UGCCUGCU | 594 | AGCAGGCA GGCTAGCTACAACGA AGTTAATT | 10039 |
| 2655 | UAGGUUUU A UCCCAAUG | 599 | CATTGGGA GGCTAGCTACAACGA AAAACCTA | 10040 |
| 2661 | UUAUCCCA A UGUUACUA | 1968 | TAGTAACA GGCTAGCTACAACGA TGGGATAA | 10041 |
| 2666 | CCAAUGUU A CUAAAUAU | 602 | ATATTTAG GGCTAGCTACAACGA AACATTGG | 10042 |
| 2671 | GUUACUAA A UAUUUGCC | 1969 | GGCAAATA GGCTAGCTACAACGA TTAGTAAC | 10043 |
| 2673 | UACUAAAU A UUUGCCCU | 604 | AGGGCAAA GGCTAGCTACAACGA ATTTAGTA | 10044 |
| 2685 | GCCCUUAG A UAAAGGGA | 1970 | TCCCTTTA GGCTAGCTACAACGA CTAAGGGC | 10045 |
| 2693 | AUAAAGGG A UCAAACCG | 1971 | CGGTTTGA GGCTAGCTACAACGA CCCTTTAT | 10046 |
| 2698 | GGGAUCAA A CCGUAUUA | 1972 | TAATACGG GGCTAGCTACAACGA TTGATCCC | 10047 |
| 2703 | CAAACCGU A UUAUCCAG | 611 | CTGGATAA GGCTAGCTACAACGA ACGGTTTG | 10048 |
| 2706 | ACCGUAUU A UCCAGAGU | 613 | ACTCTGGA GGCTAGCTACAACGA AATACGGT | 10049 |
| 2715 | UCCAGAGU A UGUAGUUA | 615 | TAACTACA GGCTAGCTACAACGA ACTCTGGA | 10050 |
| 2724 | UGUAGUUA A UCAUUACU | 1973 | AGTAATGA GGCTAGCTACAACGA TAACTACA | 10051 |
| 2727 | AGUUAAUC A UUACUUCC | 1313 | GGAAGTAA GGCTAGCTACAACGA GATTAACT | 10052 |
| 2730 | UAAUCAUU A CUUCCAGA | 621 | TCTGGAAG GGCTAGCTACAACGA AATGATTA | 10053 |
| 2738 | ACUUCCAG A CGCGACAU | 1974 | ATGTCGCG GGCTAGCTACAACGA CTGGAAGT | 10054 |
| 2743 | CAGACGCG A CAUUAUUU | 1975 | AAATAATG GGCTAGCTACAACGA CGCGTCTG | 10055 |
| 2745 | GACGCGAC A UUAUUUAC | 1317 | GTAAATAA GGCTAGCTACAACGA GTCGCGTC | 10056 |
| 2748 | GCGACAUU A UUUACACA | 625 | TGTGTAAA GGCTAGCTACAACGA AATGTCGC | 10057 |
| 2752 | CAUUAUUU A CACACUCU | 628 | AGAGTGTG GGCTAGCTACAACGA AAATAATG | 10058 |
| 2754 | UUAUUUAC A CACUCUUU | 1318 | AAAGAGTG GGCTAGCTACAACGA GTAAATAA | 10059 |
| 2756 | AUUUACAC A CUCUUUGG | 1319 | CCAAAGAG GGCTAGCTACAACGA GTGTAAAT | 10060 |
| 2774 | AGGCGGGG A UCUUAUAU | 1976 | ATATAAGA GGCTAGCTACAACGA CCCCGCCT | 10061 |
| 2779 | GGGAUCUU A UAUAAAAG | 634 | CTTTTATA GGCTAGCTACAACGA AAGATCCC | 10062 |
| 2781 | GAUCUUAU A UAAAAGAG | 635 | CTCTTTTA GGCTAGCTACAACGA ATAAGATC | 10063 |
| 2795 | GAGAGUCC A CACGUAGC | 1324 | GCTACGTG GGCTAGCTACAACGA GCACTCTC | 10064 |
| 2797 | CAGUCCAC A CGUAGCGC | 1325 | GCGCTACG GGCTAGCTACAACGA GTGGACTC | 10065 |
| 2809 | AGCGCCUC A UUUUGCGG | 1328 | CCGCAAAA GGCTAGCTACAACGA GAGGCGCT | 10066 |
| 2821 | UGCGGGUC A CCAUAUUC | 1329 | GAATATGG GGCTAGCTACAACGA GACCCGCA | 10067 |
| 2824 | GGGUCACC A UAUUCUUG | 1331 | CAAGAATA GGCTAGCTACAACGA GGTGACCC | 10068 |
| 2826 | GUCACCAU A UUCUUGGG | 644 | CCCAAGAA GGCTAGCTACAACGA ATGGTGAC | 10069 |
| 2836 | UCUUGGGA A CAAGAUCU | 1977 | AGATCTTG GGCTAGCTACAACGA TCCCAAGA | 10070 |
| 2841 | GGAACAAG A UCUACAGC | 1978 | GCTGTAGA GGCTAGCTACAACGA CTTGTTCC | 10071 |
| 2845 | CAAGAUCU A CAGCAUGG | 649 | CCATGCTG GGCTAGCTACAACGA AGATCTTG | 10072 |
| 2850 | UCUACAGC A UGGGAGGU | 1336 | ACCTCCCA GGCTAGCTACAACGA GCTGTAGA | 10073 |
| 2870 | UCUUCCAA A CCUCGAAA | 1979 | TTTCGAGG GGCTAGCTACAACGA TTGGAAGA | 10074 |
| 2883 | GAAAAGGC A UGGGGACA | 1342 | TGTCCCCA GGCTAGCTACAACGA GCCTTTTC | 10075 |
| 2889 | GCAUGGGG A CAAAUCUU | 1980 | AAGATTTG GGCTAGCTACAACGA CCCCATGC | 10076 |
| 2893 | GGGGACAA A UCUUUCUG | 1981 | CAGAAAGA GGCTAGCTACAACGA TTGTCCCC | 10077 |
| 2908 | UCUCCCCA A UCCCCUGG | 1982 | CCAGGGGA GGCTAGCTACAACGA TGGGGACA | 10078 |
| 2918 | CCCCUGGG A UUCUUCCC | 1983 | GGGAAGAA GGCTAGCTACAACGA CCCAGGGG | 10079 |
| 2929 | CUUCCCCG A UCAUCAGU | 1984 | ACTGATGA GGCTAGCTACAACGA CGGGGAAG | 10080 |
| 2932 | CCCCGAUC A UCAGUUGG | 1358 | CCAACTGA GGCTAGCTACAACGA GATCGGGG | 10081 |
| 2941 | UCAGUUGG A CCCUGCAU | 1985 | ATGCAGGG GGCTAGCTACAACGA CCAACTGA | 10082 |
| 2948 | GACCCUGC A UUCAAAGC | 1363 | GCTTTGAA GGCTAGCTACAACGA GCAGGGTC | 10083 |
| 2959 | CAAAGCCA A CUCAGUAA | 1986 | TTACTGAG GGCTAGCTACAACGA TGGCTTTG | 10084 |
| 2968 | CUCAGUAA A UCCAGAUU | 1987 | AATCTGGA GGCTAGCTACAACGA TTACTGAG | 10085 |
| 2974 | AAAUCCAG A UUGGGACC | 1988 | GGTCCCAA GGCTAGCTACAACGA CTGGATTT | 10086 |
| 2980 | AGAUUGGG A CCUCAACC | 1989 | GGTTGAGG GGCTAGCTACAACGA CCCAATCT | 10087 |
| 2986 | GGACCUCA A CCCGCACA | 1990 | TGTGCGGG GGCTAGCTACAACGA TGAGGTCC | 10088 |
| 2998 | GCACAAGG A CAACUGGC | 1991 | GCCAGTTG GGCTAGCTACAACGA CCTTGTGC | 10089 |
| 3001 | CAAGGACA A CUGGCCGG | 1992 | CCGGCCAG GGCTAGCTACAACGA TGTCCTTG | 10090 |
| 3010 | CUGGCCGG A CGCCAACA | 1993 | TGTTGGCG GGCTAGCTACAACGA CCGGCCAG | 10091 |
| 3016 | GGACGCCA A CAAGGUGG | 1994 | CCACCTTG GGCTAGCTACAACGA TGGCGTCC | 10092 |
| 3035 | GUGGGAGC A UUCGGGCC | 1384 | GGCCCGAA GGCTAGCTACAACGA GCTCCCAC | 10093 |
| 3051 | CAGGGUUC A CCCCUCCC | 1387 | GGGAGGGG GGCTAGCTACAACGA GAACCCTG | 10094 |
| 3061 | CCCUCCCC A UGGGGGAC | 1395 | GTCCCCCA GGCTAGCTACAACGA GGGGAGGG | 10095 |
| 3068 | CAUGGGGG A CUGUUGGG | 1995 | CCCAACAG GGCTAGCTACAACGA CCCCCATG | 10096 |
| 3088 | GAGCCCUC A CGCUCAGG | 1400 | CCTGAGCG GGCTAGCTACAACGA GAGGGCTC | 10097 |
| 3101 | CAGGGCCU A CUCACAAC | 683 | GTTGTGAG GGCTAGCTACAACGA AGGCCCTG | 10098 |
| 3105 | GCCUACUC A CAACUGUG | 1406 | CACAGTTG GGCTAGCTACAACGA GAGTAGGC | 10099 |
| 3108 | UACUCACA A CUGUGCCA | 1996 | TGGCACAG GGCTAGCTACAACGA TGTGAGTA | 10100 |
| 3138 | CUGCCUCC A CCAAUCGG | 1422 | CCGATTGG GGCTAGCTACAACGA GGAGGCAG | 10101 |
| 3142 | CUCCACCA A UCGGCAGU | 1997 | ACTGCCGA GGCTAGCTACAACGA TGGTGGAG | 10102 |
| 3165 | GGCAGCCU A CUCCCUUA | 691 | TAAGGGAG GGCTAGCTACAACGA AGGCTGCC | 10103 |
| 3173 | ACUCCCUU A UCUCCACC | 694 | GGTGGAGA GGCTAGCTACAACGA AAGGGAGT | 10104 |
| 3179 | UUAUCUCC A CCUCUAAG | 1436 | CTTAGAGG GGCTAGCTACAACGA GGAGATAA | 10105 |
| 3190 | UCUAAGGG A CACUCAUC | 1998 | GATGAGTG GGCTAGCTACAACGA CCCTTAGA | 10106 |
| 3192 | UAAGGGAC A CUCAUCCU | 1440 | AGGATGAG GGCTAGCTACAACGA GTCCCTTA | 10107 |
| 3196 | GGACACUC A UCCUCAGG | 1442 | CCTGAGGA GGCTAGCTACAACGA GAGTGTCC | 10108 |
| 3207 | CUCAGGCC A UGCAGUGG | 1447 | CCACTGCA GGCTAGCTACAACGA GGCCTGAG | 10109 |

Input Sequence = AF100308.
Cut Site = YG/M or UG/U.
Stem Length = 8.
Core Sequence = GGCTAGCTACAACGA
AF100308 (Hepatitis B virus strain 2-18, 3215 bp)

TABLE X

HUMAN HBV AMBERZYME AND SUBSTRATE SEQUENCE

| | | | | |
|---|---|---|---|---|
| Pos | Substrate | Seq ID | Amberzyme | Seq ID |

| | | | | |
|---|---|---|---|---|
| 61 | ACUUUCCU G CUGGUGGC | 1448 | GCCACCAG GGAGGAAACUCC CU UCAAGGACAUCGUCCGGG AGGAAAGU | 10110 |
| 87 | GGAACAGU G AGCCCUGC | 1449 | GCAGGGCU GGAGGAAACUCC CU UCAAGGACAUCGUCCGGG ACUGUUCC | 10111 |
| 94 | UGAGCCCU G CUCAGAAU | 1450 | AUUCUGAG GGAGGAAACUCC CU UCAAGGACAUCGUCCGGG AGGGCUCA | 10112 |
| 112 | CUGUCUCU G CCAUAUCG | 1451 | CGAUAUGG GGAGGAAACUCC CU UCAAGGACAUCGUCCGGG AGAGACAG | 10113 |
| 132 | AUCUUAUC G AAGACUGG | 1452 | CCAGUCUU GGAGGAAACUCC CU UCAAGGACAUCGUCCGGG GAUAAGAU | 10114 |
| 153 | CCUGUACC G AACAUGGA | 1453 | UCCAUGUU GGAGGAAACUCC CU UCAAGGACAUCGUCCGGG GGUACAGG | 10115 |
| 169 | AGAACAUC G CAUCAGGA | 1454 | UCCUGAUG GGAGGAAACUCC CU UCAAGGACAUCGUCCGGG GAUGUUCU | 10116 |
| 192 | GGACCCCU G CUCGUGUU | 1455 | AACACGAG GGAGGAAACUCC CU UCAAGGACAUCGUCCGGG AGGGGUCC | 10117 |
| 222 | UUCUUGUU G ACAAAAAU | 1456 | AUUUUUGU GGAGGAAACUCC CU UCAAGGACAUCGUCCGGG AACAAGAA | 10118 |
| 315 | CAAAAUUC G CAGUCCCA | 1457 | UGGGACUG GGAGGAAACUCC CU UCAAGGACAUCGUCCGGG GAAUUUUG | 10119 |
| 374 | UGGUUAUC G CUGGAUGU | 1458 | ACAUCCAG GGAGGAAACUCC CU UCAAGGACAUCGUCCGGG GAUAACCA | 10120 |
| 387 | AUGUGUCU G CGGCGUUU | 1459 | AAACGCCG GGAGGAAACUCC CU UCAAGGACAUCGUCCGGG AGACACAU | 10121 |
| 410 | CUUCCUCU G CAUCCUGC | 1460 | GCAGGAUG GGAGGAAACUCC CU UCAAGGACAUCGUCCGGG AGAGGAAG | 10122 |
| 417 | UGCAUCCU G CUGCUAUG | 1461 | CAUAGCAG GGAGGAAACUCC CU UCAAGGACAUCGUCCGGG AGGAUGCA | 10123 |
| 420 | AUCCUGCU G CUAUGCCU | 1462 | AGGCAUAG GGAGGAAACUCC CU UCAAGGACAUCGUCCGGG AGCAGGAU | 10124 |
| 425 | GCUGCUAU G CCUCAUCU | 1463 | AGAUGAGG GGAGGAAACUCC CU UCAAGGACAUCGUCCGGG AUAGCAGC | 10125 |
| 468 | GGUAUGUU G CCCGUUUG | 1464 | CAAACGGG GGAGGAAACUCC CU UCAAGGACAUCGUCCGGG AACAUACC | 10126 |
| 518 | CGGACCAU G CAAAACCU | 1465 | AGGUUUUG GGAGGAAACUCC CU UCAAGGACAUCGUCCGGG AUGGUCCG | 10127 |
| 527 | CAAAACCU G CACAACUC | 1466 | GAGUUGUG GGAGGAAACUCC CU UCAAGGACAUCGUCCGGG AGGUUUUG | 10128 |
| 538 | CAACUCCU G CUCAAGGA | 1467 | UCCUUGAG GGAGGAAACUCC CU UCAAGGACAUCGUCCGGG AGGAGUUG | 10129 |
| 569 | CUCAUGUU G CUGUACAA | 1468 | UUGUACAG GGAGGAAACUCC CU UCAAGGACAUCGUCCGGG AACAUGAG | 10130 |
| 596 | CGGAAACU G CACCUGUA | 1469 | UACAGGUG GGAGGAAACUCC CU UCAAGGACAUCGUCCGGG AGUUUCCG | 10131 |
| 631 | GGGCUUUC G CAAAAUAC | 1470 | GUAUUUUG GGAGGAAACUCC CU UCAAGGACAUCGUCCGGG GAAAGCCC | 10132 |
| 687 | UUACUAGU G CCAUUUGU | 1471 | ACAAAUGG GGAGGAAACUCC CU UCAAGGACAUCGUCCGGG ACUAGUAA | 10133 |
| 747 | AUAUGGAU G AUGUGGUU | 1472 | AACCACAU GGAGGAAACUCC CU UCAAGGACAUCGUCCGGG AUCCAUAU | 10134 |
| 783 | AACAUCUU G AGUCCCUU | 1473 | AAGGGACU GGAGGAAACUCC CU UCAAGGACAUCGUCCGGG AAGAUGUU | 10135 |
| 795 | CCCUUUAU G CCGCUGUU | 1474 | AACAGCGG GGAGGAAACUCC CU UCAAGGACAUCGUCCGGG AUAAAGGG | 10136 |
| 798 | UUUAUGCC G CUGUUACC | 1475 | GGUAACAG GGAGGAAACUCC CU UCAAGGACAUCGUCCGGG GGCAUAAA | 10137 |
| 911 | GGCACAUU G CCACAGGA | 1476 | UCCUGUGG GGAGGAAACUCC CU UCAAGGACAUCGUCCGGG AAUGUGCC | 10138 |
| 978 | GGCCUAUU G AUUGGAAA | 1477 | UUUCCAAU GGAGGAAACUCC CU UCAAGGACAUCGUCCGGG AAUAGGCC | 10139 |
| 997 | AUGUCAAC G AAUUGUGG | 1478 | CCACAAUU GGAGGAAACUCC CU UCAAGGACAUCGUCCGGG GUUGACAU | 10140 |
| 1020 | UGGGGUUU G CCGCCCCU | 1479 | AGGGGCGG GGAGGAAACUCC CU UCAAGGACAUCGUCCGGG AAACCCCA | 10141 |
| 1023 | GGUUUGCC G CCCCUUUC | 1480 | GAAAGGGG GGAGGAAACUCC CU UCAAGGACAUCGUCCGGG GGCAAACC | 10142 |
| 1034 | CCUUUCAC G CAAUGUGG | 1481 | CCACAUUG GGAGGAAACUCC CU UCAAGGACAUCGUCCGGG GUGAAAGG | 10143 |
| 1050 | GAUAUUCU G CUUUAAUG | 1482 | CAUUAAAG GGAGGAAACUCC CU UCAAGGACAUCGUCCGGG AGAAUAUC | 10144 |
| 1058 | GCUUUAAU G CCUUUAUA | 1483 | UAUAAAGG GGAGGAAACUCC CU UCAAGGACAUCGUCCGGG AUUAAAGC | 10145 |
| 1068 | CUUUAUAU G CAUGCAUA | 1484 | UAUGCAUG GGAGGAAACUCC CU UCAAGGACAUCGUCCGGG AUAUAAAG | 10146 |
| 1072 | AUAUGCAU G CAUACAAG | 1485 | CUUGUAUG GGAGGAAACUCC CU UCAAGGACAUCGUCCGGG AUGCAUAU | 10147 |
| 1103 | ACUUUCUC G CCAACUUA | 1486 | UAAGUUGG GGAGGAAACUCC CU UCAAGGACAUCGUCCGGG GAGAAAGU | 10148 |
| 1139 | CAGUAUGU G AACCUUUA | 1487 | UAAAGGUU GGAGGAAACUCC CU UCAAGGACAUCGUCCGGG ACAUACUG | 10149 |
| 1155 | ACCCCGUU G CUCGGCAA | 1488 | UUGCCGAG GGAGGAAACUCC CU UCAAGGACAUCGUCCGGG AACGGGGU | 10150 |
| 1177 | UGGUCUAU G CCAAGUGU | 1489 | ACACUUGG GGAGGAAACUCC CU UCAAGGACAUCGUCCGGG AUAGACCA | 10151 |
| 1188 | AAGUGUUU G CUGACGCA | 1490 | UGCGUCAG GGAGGAAACUCC CU UCAAGGACAUCGUCCGGG AAACACUU | 10152 |
| 1191 | UGUUUGCU G ACGCAACC | 1491 | GGUUGCGU GGAGGAAACUCC CU UCAAGGACAUCGUCCGGG AGCAAACA | 10153 |
| 1194 | UUGCUGAC G CAACCCCC | 1492 | GGGGGUUG GGAGGAAACUCC CU UCAAGGACAUCGUCCGGG GUCAGCAA | 10154 |
| 1234 | CCAUCAGC G CAUGCGUG | 1493 | CACGCAUG GGAGGAAACUCC CU UCAAGGACAUCGUCCGGG GCUGAUGG | 10155 |
| 1238 | CAGCGCAU G CGUGGAAC | 1494 | GUUCCACG GGAGGAAACUCC CU UCAAGGACAUCGUCCGGG AUGCGCUG | 10156 |
| 1262 | UCUCCUCU G CCGAUCCA | 1495 | UGGAUCGG GGAGGAAACUCC CU UCAAGGACAUCGUCCGGG AGAGGAGA | 10157 |
| 1265 | CCUCUGCC G AUCCAUAC | 1496 | GUAUGGAU GGAGGAAACUCC CU UCAAGGACAUCGUCCGGG GGCAGAGG | 10158 |
| 1275 | UCCAUACC G CGGAACUC | 1497 | GAGUUCCG GGAGGAAACUCC CU UCAAGGACAUCGUCCGGG GGUAUGGA | 10159 |
| 1290 | UCCUAGCC G CUUGUUUU | 1498 | AAAACAAG GGAGGAAACUCC CU UCAAGGACAUCGUCCGGG GGCUAGGA | 10160 |
| 1299 | CUUGUUUU G CUCGCAGC | 1499 | GCUGCGAG GGAGGAAACUCC CU UCAAGGACAUCGUCCGGG AAAACAAG | 10161 |
| 1303 | UUUUGCUC G CAGCAGGU | 1500 | ACCUGCUG GGAGGAAACUCC CU UCAAGGACAUCGUCCGGG GAGCAAAA | 10162 |
| 1335 | UCGGGACU G ACAAUUCU | 1501 | AGAAUUGU GGAGGAAACUCC CU UCAAGGACAUCGUCCGGG AGUCCCGA | 10163 |
| 1349 | UCUGUCGU G CUCUCCCG | 1502 | CGGGAGAG GGAGGAAACUCC CU UCAAGGACAUCGUCCGGG ACGACAGA | 10164 |
| 1357 | GCUCUCCC G CAAAUAUA | 1503 | UAUAUUUG GGAGGAAACUCC CU UCAAGGACAUCGUCCGGG GGGAGAGC | 10165 |
| 1382 | CCAUGGCU G CUAGGCUG | 1504 | CAGCCUAG GGAGGAAACUCC CU UCAAGGACAUCGUCCGGG AGCCAUGG | 10166 |
| 1392 | UAGGCUGU G CUGCCAAC | 1505 | GUUGGCAG GGAGGAAACUCC CU UCAAGGACAUCGUCCGGG ACAGCCUA | 10167 |
| 1395 | GCUGUGCU G CCAACUGG | 1506 | CCAGUUGG GGAGGAAACUCC CU UCAAGGACAUCGUCCGGG AGCACAGC | 10168 |
| 1411 | GAUCCUAC G CGGGACGU | 1507 | ACGUCCCG GGAGGAAACUCC CU UCAAGGACAUCGUCCGGG GUAGGAUC | 10169 |
| 1442 | CCGUCGGC G CUGAAUCC | 1508 | GGAUUCAG GGAGGAAACUCC CU UCAAGGACAUCGUCCGGG GCCGACGG | 10170 |
| 1445 | UCGGCGCU G AAUCCCGC | 1509 | GCGGGAUU GGAGGAAACUCC CU UCAAGGACAUCGUCCGGG AGCGCCGA | 10171 |
| 1452 | UGAAUCCC G CGGACGAC | 1510 | GUCGUCCG GGAGGAAACUCC CU UCAAGGACAUCGUCCGGG GGGAUUCA | 10172 |
| 1458 | CCGCGGAC G ACCCCUCC | 1511 | GGAGGGGU GGAGGAAACUCC CU UCAAGGACAUCGUCCGGG GUCCGCGG | 10173 |
| 1474 | CCGGGGCC G CUUGGGGC | 1512 | GCCCCAAG GGAGGAAACUCC CU UCAAGGACAUCGUCCGGG GGCCCCGG | 10174 |
| 1489 | GCUCUACC G CCCGCUUC | 1513 | GAAGCGGG GGAGGAAACUCC CU UCAAGGACAUCGUCCGGG GGUAGAGC | 10175 |
| 1493 | UACCGCCC G CUUCUCCG | 1514 | CGGAGAAG GGAGGAAACUCC CU UCAAGGACAUCGUCCGGG GGGCGGUA | 10176 |
| 1501 | GCUUCUCC G CCUAUUGU | 1515 | ACAAUAGG GGAGGAAACUCC CU UCAAGGACAUCGUCCGGG GGAGAAGC | 10177 |
| 1513 | AUUGUACC G ACCGUCCA | 1516 | UGGACGGU GGAGGAAACUCC CU UCAAGGACAUCGUCCGGG GGUACAAU | 10178 |
| 1528 | CACGGGGC G CACCUCUC | 1517 | GAGAGGUG GGAGGAAACUCC CU UCAAGGACAUCGUCCGGG GCCCCGUG | 10179 |
| 1542 | CUCUUUAC G CGGACUCC | 1518 | GGAGUCCG GGAGGAAACUCC CU UCAAGGACAUCGUCCGGG GUAAAGAG | 10180 |
| 1559 | CCGUCUGU G CCUUCUCA | 1519 | UGAGAAGG GGAGGAAACUCC CU UCAAGGACAUCGUCCGGG ACAGACGG | 10181 |
| 1571 | UCUCAUCU G CCGGACCG | 1520 | CGGUCCGG GGAGGAAACUCC CU UCAAGGACAUCGUCCGGG AGAUGAGA | 10182 |
| 1583 | GACCGUGU G CACUUCGC | 1521 | GCGAAGUG GGAGGAAACUCC CU UCAAGGACAUCGUCCGGG ACACGGUC | 10183 |
| 1590 | UGCACUUC G CUUCACCU | 1522 | AGGUGAAG GGAGGAAACUCC CU UCAAGGACAUCGUCCGGG GAAGUGCA | 10184 |
| 1601 | UCACCUCU G CACGUCGC | 1523 | GCGACGUG GGAGGAAACUCC CU UCAAGGACAUCGUCCGGG AGAGGUGA | 10185 |
| 1608 | UGCACGUC G CAUGGAGA | 1524 | UCUCCAUG GGAGGAAACUCC CU UCAAGGACAUCGUCCGGG GACGUGCA | 10186 |
| 1624 | ACCACCGU G AACGCCCA | 1525 | UGGGCGUU GGAGGAAACUCC CU UCAAGGACAUCGUCCGGG ACGGUGGU | 10187 |
| 1628 | CCGUGAAC G CCCACAGG | 1526 | CCUGUGGG GGAGGAAACUCC CU UCAAGGACAUCGUCCGGG GUUCACGG | 10188 |
| 1642 | AGGAACCU G CCCAAGGU | 1527 | ACCUUGGG GGAGGAAACUCC CU UCAAGGACAUCGUCCGGG AGGUUCCU | 10189 |
| 1654 | AAGGUCUU G CAUAAGAG | 1528 | CUCUUAUG GGAGGAAACUCC CU UCAAGGACAUCGUCCGGG AAGACCUU | 10190 |
| 1690 | AUGUCAAC G ACCGACCU | 1529 | AGGUCGGU GGAGGAAACUCC CU UCAAGGACAUCGUCCGGG GUUGACAU | 10191 |
| 1694 | CAACGACC G ACCUUGAG | 1530 | CUCAAGGU GGAGGAAACUCC CU UCAAGGACAUCGUCCGGG GGUCGUUG | 10192 |
| 1700 | CCGACCUU G AGGCAUAC | 1531 | GUAUGCCU GGAGGAAACUCC CU UCAAGGACAUCGUCCGGG AAGGUCGG | 10193 |
| 1730 | UCUUUAAU G AGUGGGAG | 1532 | CUCCCACU GGAGGAAACUCC CU UCAAGGACAUCGUCCGGG AUUAAACA | 10194 |
| 1818 | AGCACCAU G CAACUUUU | 1533 | AAAAGUUG GGAGGAAACUCC CU UCAAGGACAUCGUCCGGG AUGGUGCU | 10195 |
| 1835 | UCACCUCU G CCUAAUCA | 1534 | UGAUUAGG GGAGGAAACUCC CU UCAAGGACAUCGUCCGGG AGAGGUGA | 10196 |
| 1883 | CAAGCUGU G CCUUGGGU | 1535 | ACCCAAGG GGAGGAAACUCC CU UCAAGGACAUCGUCCGGG ACAGCUUG | 10197 |
| 1912 | UGGACAUU G ACCCGUAU | 1536 | AUACGGGU GGAGGAAACUCC CU UCAAGGACAUCGUCCGGG AAUGUCCA | 10198 |
| 1959 | UCUUUUUU G CCUUCUGA | 1537 | UCAGAAGG GGAGGAAACUCC CU UCAAGGACAUCGUCCGGG AAAAAAGA | 10199 |
| 1966 | UGCCUUCU G ACUUCUUU | 1538 | AAAGAAGU GGAGGAAACUCC CU UCAAGGACAUCGUCCGGG AGAAGGCA | 10200 |
| 1985 | UUCUAUUC G AGAUCUCC | 1539 | GGAGAUCU GGAGGAAACUCC CU UCAAGGACAUCGUCCGGG GAAUAGAA | 10201 |
| 1996 | AUCUCCUC G ACACCGCC | 1540 | GGCGGUGU GGAGGAAACUCC CU UCAAGGACAUCGUCCGGG GAGGAGAU | 10202 |
| 2002 | UCGACACC G CCUCUGCU | 1541 | AGCAGAGG GGAGGAAACUCC CU UCAAGGACAUCGUCCGGG GGUGUCGA | 10203 |
| 2008 | CCGCCUCU G CUCUGUAU | 1542 | AUACAGAG GGAGGAAACUCC CU UCAAGGACAUCGUCCGGG AGAGGCGG | 10204 |
| 2092 | GUUGGGGU G AGUUGAUG | 1543 | CAUCAACU GGAGGAAACUCC CU UCAAGGACAUCGUCCGGG ACCCCAAC | 10205 |
| 2097 | GGUGAGUU G AUGAAUCU | 1544 | AGAUUCAU GGAGGAAACUCC CU UCAAGGACAUCGUCCGGG AACUCACC | 10206 |
| 2100 | GAGUUGAU G AAUCUAGC | 1545 | GCUAGAUU GGAGGAAACUCC CU UCAAGGACAUCGUCCGGG AUCAACUC | 10207 |
| 2237 | UUUUGGGC G AGAAACUG | 1546 | CAGUUUCU GGAGGAAACUCC CU UCAAGGACAUCGUCCGGG GCCCAAAA | 10208 |
| 2251 | CUGUUCUU G AAUAUUUG | 1547 | CAAAUAUU GGAGGAAACUCC CU UCAAGGACAUCGUCCGGG AAGAACAG | 10209 |
| 2282 | GUGGAUUC G CACUCCUC | 1548 | GAGGAGUG GGAGGAAACUCC CU UCAAGGACAUCGUCCGGG GAAUCCAC | 10210 |
| 2293 | CUCCUCCU G CAUAUAGA | 1549 | UCUAUAUG GGAGGAAACUCC CU UCAAGGACAUCGUCCGGG AGGAGGAG | 10211 |
| 2311 | CACCAAAU G CCCCUAUC | 1550 | GAUAGGGG GGAGGAAACUCC CU UCAAGGACAUCGUCCGGG AUUUGGUG | 10212 |
| 2354 | UGUUAGAC G AAGAGGCA | 1551 | UGCCUCUU GGAGGAAACUCC CU UCAAGGACAUCGUCCGGG GUCUAACA | 10213 |
| 2388 | ACUCCCUC G CCUCGCAG | 1552 | CUGCGAGG GGAGGAAACUCC CU UCAAGGACAUCGUCCGGG GAGGGAGU | 10214 |
| 2393 | CUCGCCUC G CAGACGAA | 1553 | UUCGUCUG GGAGGAAACUCC CU UCAAGGACAUCGUCCGGG GAGGCGAG | 10215 |
| 2399 | UCGCAGAC G AAGGUCUC | 1554 | GAGACCUU GGAGGAAACUCC CU UCAAGGACAUCGUCCGGG GUCUGCGA | 10216 |
| 2412 | UCUCAAUC G CCGCGUCG | 1555 | CGACGCGG GGAGGAAACUCC CU UCAAGGACAUCGUCCGGG GAUUGAGA | 10217 |
| 2415 | CAAUCGCC G CGUCGCAG | 1556 | CUGCGACG GGAGGAAACUCC CU UCAAGGACAUCGUCCGGG GGCGAUUG | 10218 |
| 2420 | GCCGCGUC G CAGAAGAU | 1557 | AUCUUCUG GGAGGAAACUCC CU UCAAGGACAUCGUCCGGG GACGCGGC | 10219 |
| 2514 | GGUACCUU G CUUUAAUC | 1558 | GAUUAAAG GGAGGAAACUCC CU UCAAGGACAUCGUCCGGG AAGGUACC | 10220 |
| 2549 | CUUUUCCU G ACAUUCAU | 1559 | AUGAAUGU GGAGGAAACUCC CU UCAAGGACAUCGUCCGGG AGGAAAAG | 10221 |
| 2560 | AUUCAUUU G CAGGAGGA | 1560 | UCCUCCUG GGAGGAAACUCC CU UCAAGGACAUCGUCCGGG AAAUGAAU | 10222 |
| 2576 | ACAUUGUU G AUAGAUGU | 1561 | ACAUCUAU GGAGGAAACUCC CU UCAAGGACAUCGUCCGGG AACAAUGU | 10223 |
| 2615 | CAGUAAAU G AAAACAGG | 1562 | CCUGUUUU GGAGGAAACUCC CU UCAAGGACAUCGUCCGGG AUUUACUG | 10224 |
| 2641 | UUAACUAU G CCUGCUAG | 1563 | CUAGCAGG GGAGGAAACUCC CU UCAAGGACAUCGUCCGGG AUAGUUAA | 10225 |
| 2645 | CUAUGCCU G CUAGGUUU | 1564 | AAACCUAG GGAGGAAACUCC CU UCAAGGACAUCGUCCGGG AGGCAUAG | 10226 |
| 2677 | AAAUAUUU G CCCUUAGA | 1565 | UCUAAGGG GGAGGAAACUCC CU UCAAGGACAUCGUCCGGG AAAUAUUU | 10227 |
| 2740 | UUCCAGAC G CGACAUUA | 1566 | UAAUGUCG GGAGGAAACUCC CU UCAAGGACAUCGUCCGGG GUCUGGAA | 10228 |
| 2742 | CCAGACGC G ACAUUAUU | 1567 | AAUAAUGU GGAGGAAACUCC CU UCAAGGACAUCGUCCGGG GCGUCUGG | 10229 |
| 2804 | CACGUAGC G CCUCAUUU | 1568 | AAAUGAGG GGAGGAAACUCC CU UCAAGGACAUCGUCCGGG GCUACGUG | 10230 |
| 2814 | CUCAUUUU G CGGGUCAC | 1569 | GUGACCCG GGAGGAAACUCC CU UCAAGGACAUCGUCCGGG AAAAUGAG | 10231 |
| 2875 | CAAACCUC G AAAAGGCA | 1570 | UGCCUUUU GGAGGAAACUCC CU UCAAGGACAUCGUCCGGG GAGGUUUG | 10232 |
| 2928 | UCUUCCCC G AUCAUCAG | 1571 | CUGAUGAU GGAGGAAACUCC CU UCAAGGACAUCGUCCGGG GGGGAAGA | 10233 |
| 2946 | UGGACCCU G CAUUCAAA | 1572 | UUUGAAUG GGAGGAAACUCC CU UCAAGGACAUCGUCCGGG AGGGUCCA | 10234 |
| 2990 | CUCAACCC G CACAAGGA | 1573 | UCCUUGUG GGAGGAAACUCC CU UCAAGGACAUCGUCCGGG GGGUUGAG | 10235 |
| 3012 | GGCCGGAC G CCAACAAG | 1574 | CUUGUUGG GGAGGAAACUCC CU UCAAGGACAUCGUCCGGG GUCCGGCC | 10236 |
| 3090 | GCCCUCAC G CUCAGGGC | 1575 | GCCCUGAG GGAGGAAACUCC CU UCAAGGACAUCGUCCGGG GUGAGGGC | 10237 |
| 3113 | ACAACUGU G CCAGCAGC | 1576 | GCUGCUGG GGAGGAAACUCC CU UCAAGGACAUCGUCCGGG ACAGUUGU | 10238 |
| 3132 | CUCCUCCU G CCUCCACC | 1577 | GGUGGAGG GGAGGAAACUCC CU UCAAGGACAUCGUCCGGG AGGAGGAG | 10239 |
| 51 | AGGGCCCU G UACUUUCC | 1578 | GGAAAGUA GGAGGAAACUCC CU UCAAGGACAUCGUCCGGG AGGGCCCU | 10240 |
| 106 | AGAAUACU G UCUCUGCC | 1579 | GGCAGAGA GGAGGAAACUCC CU UCAAGGACAUCGUCCGGG AGUAUUCU | 10241 |
| 148 | GGGACCCU G UACCGAAC | 1580 | GUUCGGUA GGAGGAAACUCC CU UCAAGGACAUCGUCCGGG AGGGUCCC | 10242 |
| 198 | CUGCUCGU G UUACAGGC | 1581 | GCCUGUAA GGAGGAAACUCC CU UCAAGGACAUCGUCCGGG ACGAGCAG | 10243 |
| 219 | UUUUUCUU G UUUGUCAA | 1582 | UUUGUCAA GGAGGAAACUCC CU UCAAGGACAUCGUCCGGG AAGAAAAA | 10244 |
| 297 | ACACCCGU G UGUCUUGG | 1583 | CCAAGACA GGAGGAAACUCC CU UCAAGGACAUCGUCCGGG ACGGGUGU | 10245 |
| 299 | ACCCGUGU G UCUUGGCC | 1584 | GGCCAAGA GGAGGAAACUCC CU UCAAGGACAUCGUCCGGG ACACGGGU | 10246 |
| 347 | ACCAACCU G UUGUCCUC | 1585 | GAGGACAA GGAGGAAACUCC CU UCAAGGACAUCGUCCGGG AGGUUGGU | 10247 |
| 350 | AACCUGUU G UCCUCCAA | 1586 | UUGGAGGA GGAGGAAACUCC CU UCAAGGACAUCGUCCGGG AACAGGUU | 10248 |
| 362 | UCCAAUUU G UCCUGGUU | 1587 | AACCAGGA GGAGGAAACUCC CU UCAAGGACAUCGUCCGGG AAAUUGGA | 10249 |
| 381 | CGCUGGAU G UGUCUGCG | 1588 | CGCAGACA GGAGGAAACUCC CU UCAAGGACAUCGUCCGGG AUCCAGCG | 10250 |
| 383 | CUGGAUGU G UCUGCGGC | 1589 | GCCGCAGA GGAGGAAACUCC CU UCAAGGACAUCGUCCGGG ACAUCCAG | 10251 |
| 438 | AUCUUCUU G UUGGUUCU | 1590 | AGAACCAA GGAGGAAACUCC CU UCAAGGACAUCGUCCGGG AAGAAGAU | 10252 |
| 465 | CAAGGUAU G UUGCCCGU | 1591 | ACGGGCAA GGAGGAAACUCC CU UCAAGGACAUCGUCCGGG AUACCUUG | 10253 |
| 476 | GCCCGUUU G UCCUCUAA | 1592 | UUAGAGGA GGAGGAAACUCC CU UCAAGGACAUCGUCCGGG AAACGGGC | 10254 |
| 555 | ACCUCUAU G UUUCCCUC | 1593 | GAGGGAAA GGAGGAAACUCC CU UCAAGGACAUCGUCCGGG AUAGAGGU | 10255 |
| 566 | UCCCUCAU G UUGCUGUA | 1594 | UACAGCAA GGAGGAAACUCC CU UCAAGGACAUCGUCCGGG AUGAGGGA | 10256 |
| 572 | AUGUUGCU G UACAAAAC | 1595 | GUUUUGUA GGAGGAAACUCC CU UCAAGGACAUCGUCCGGG AGCAACAU | 10257 |
| 602 | CUGCACCU G UAUUCCCA | 1596 | UGGGAAUA GGAGGAAACUCC CU UCAAGGACAUCGUCCGGG AGGUGCAG | 10258 |
| 694 | UGCCAUUU G UUCAGUGG | 1597 | CCACUGAA GGAGGAAACUCC CU UCAAGGACAUCGUCCGGG AAAUGGCA | 10259 |
| 724 | CCCCCACU G UCUGGCUU | 1598 | AAGCCAGA GGAGGAAACUCC CU UCAAGGACAUCGUCCGGG AGUGGGGG | 10260 |
| 750 | UGGAUGAU G UGGUUUUG | 1599 | CAAAACCA GGAGGAAACUCC CU UCAAGGACAUCGUCCGGG AUCAUCCA | 10261 |
| 771 | CCAAGUCU G UACAACAU | 1600 | AUGUUGUA GGAGGAAACUCC CU UCAAGGACAUCGUCCGGG AGACUUGG | 10262 |
| 801 | AUGCCGCU G UUACCAAU | 1601 | AUUGGUAA GGAGGAAACUCC CU UCAAGGACAUCGUCCGGG AGCGGCAU | 10263 |
| 818 | UUUCUUUU G UCUUUGGG | 1602 | CCCAAAGA GGAGGAAACUCC CU UCAAGGACAUCGUCCGGG AAAAGAAA | 10264 |
| 888 | UGGGAUAU G UAAUUGGG | 1603 | CCCAAUUA GGAGGAAACUCC CU UCAAGGACAUCGUCCGGG AUAUCCCA | 10265 |
| 927 | AACAUAUU G UACAAAAA | 1604 | UUUUUGUA GGAGGAAACUCC CU UCAAGGACAUCGUCCGGG AAUAUGUU | 10266 |
| 944 | AUCAAAAU G UGUUUUAG | 1605 | CUAAAACA GGAGGAAACUCC CU UCAAGGACAUCGUCCGGG AUUUUGAU | 10267 |
| 946 | CAAAAUGU G UUUUAGGA | 1606 | UCCUAAAA GGAGGAAACUCC CU UCAAGGACAUCGUCCGGG ACAUUUUG | 10268 |
| 963 | AACUUCCU G UAAACAGG | 1607 | CCUGUUUA GGAGGAAACUCC CU UCAAGGACAUCGUCCGGG AGGAAGUU | 10269 |
| 991 | GAAAGUAU G UCAACGAA | 1608 | UUCGUUGA GGAGGAAACUCC CU UCAAGGACAUCGUCCGGG AUACUUUC | 10270 |
| 1002 | AACGAAUU G UGGGUCUU | 1609 | AAGACCCA GGAGGAAACUCC CU UCAAGGACAUCGUCCGGG AAUUCGUU | 10271 |
| 1039 | CACGCAAU G UGGAUAUU | 1610 | AAUAUCCA GGAGGAAACUCC CU UCAAGGACAUCGUCCGGG AUUGCGUG | 10272 |
| 1137 | AACAGUAU G UGAACCUU | 1611 | AAGGUUCA GGAGGAAACUCC CU UCAAGGACAUCGUCCGGG AUACUGUU | 10273 |
| 1184 | UGCCAAGU G UUUGCUGA | 1612 | UCAGCAAA GGAGGAAACUCC CU UCAAGGACAUCGUCCGGG ACUUGGCA | 10274 |
| 1251 | GAACCUUU G UGUCUCCU | 1613 | AGGAGACA GGAGGAAACUCC CU UCAAGGACAUCGUCCGGG AAAGGUUC | 10275 |
| 1253 | ACCUUUGU G UCUCCUCU | 1614 | AGAGGAGA GGAGGAAACUCC CU UCAAGGACAUCGUCCGGG ACAAAGGU | 10276 |
| 1294 | AGCCGCUU G UUUUGCUC | 1615 | GAGCAAAA GGAGGAAACUCC CU UCAAGGACAUCGUCCGGG AAGCGGCU | 10277 |
| 1344 | ACAAUUCU G UCGUGCUC | 1616 | GAGCACGA GGAGGAAACUCC CU UCAAGGACAUCGUCCGGG AGAAUUGU | 10278 |
| 1390 | GCUAGGCU G UGCUGCCA | 1617 | UGGCAGCA GGAGGAAACUCC CU UCAAGGACAUCGUCCGGG AGCCUAGC | 10279 |
| 1425 | CGUCCUUU G UUUACGUC | 1618 | GACGUAAA GGAGGAAACUCC CU UCAAGGACAUCGUCCGGG AAAGGACG | 10280 |
| 1508 | CGCCUAUU G UACCGACC | 1619 | GGUCGGUA GGAGGAAACUCC CU UCAAGGACAUCGUCCGGG AAUAGGCG | 10281 |
| 1557 | CCCCGUCU G UGCCUUCU | 1620 | AGAAGGCA GGAGGAAACUCC CU UCAAGGACAUCGUCCGGG AGACGGGG | 10282 |
| 1581 | CGGACCGU G UGCACUUC | 1621 | GAAGUGCA GGAGGAAACUCC CU UCAAGGACAUCGUCCGGG ACGGUCCG | 10283 |
| 1684 | UCAGCAAU G UCAACGAC | 1622 | GUCGUUGA GGAGGAAACUCC CU UCAAGGACAUCGUCCGGG AUUGCUGA | 10284 |
| 1719 | CAAAGACU G UGUGUUUA | 1623 | UAAACACA GGAGGAAACUCC CU UCAAGGACAUCGUCCGGG AGUCUUUG | 10285 |
| 1721 | AAGACUGU G UGUUUAAU | 1624 | AUUAAACA GGAGGAAACUCC CU UCAAGGACAUCGUCCGGG ACAGUCUU | 10286 |
| 1723 | GACUGUGU G UUUAAUGA | 1625 | UCAUUAAA GGAGGAAACUCC CU UCAAGGACAUCGUCCGGG ACACAGUC | 10287 |
| 1772 | AGGUCUUU G UACUAGGA | 1626 | UCCUAGUA GGAGGAAACUCC CU UCAAGGACAUCGUCCGGG AAAGACCU | 10288 |
| 1785 | AGGAGGCU G UAGGCAUA | 1627 | UAUGCCUA GGAGGAAACUCC CU UCAAGGACAUCGUCCGGG AGCCUCCU | 10289 |
| 1801 | AAAUUGGU G UGUUCACC | 1628 | GGUGAACA GGAGGAAACUCC CU UCAAGGACAUCGUCCGGG ACCAAUUU | 10290 |
| 1803 | AUUGGUGU G UUCACCAG | 1629 | CUGGUGAA GGAGGAAACUCC CU UCAAGGACAUCGUCCGGG ACACCAAU | 10291 |
| 1850 | CAUCUCAU G UUCAUGUC | 1630 | GACAUGAA GGAGGAAACUCC CU UCAAGGACAUCGUCCGGG AUGAGAUG | 10292 |
| 1856 | AUGUUCAU G UCCUACUG | 1631 | CAGUAGGA GGAGGAAACUCC CU UCAAGGACAUCGUCCGGG AUGAACAU | 10293 |
| 1864 | GUCCUACU G UUCAAGCC | 1632 | GGCUUGAA GGAGGAAACUCC CU UCAAGGACAUCGUCCGGG AGUAGGAC | 10294 |
| 1881 | UCCAAGCU G UGCCUUGG | 1633 | CCAAGGCA GGAGGAAACUCC CU UCAAGGACAUCGUCCGGG AGCUUGGA | 10295 |
| 1939 | GAGCUUCU G UGGAGUUA | 1634 | UAACUCCA GGAGGAAACUCC CU UCAAGGACAUCGUCCGGG AGAAGCUC | 10296 |
| 2013 | UCUGCUCU G UAUCGGGG | 1635 | CCCCGAUA GGAGGAAACUCC CU UCAAGGACAUCGUCCGGG AGAGCAGA | 10297 |
| 2045 | GGAACAUU G UUCACCUC | 1636 | GAGGUGAA GGAGGAAACUCC CU UCAAGGACAUCGUCCGGG AAUGUUCC | 10298 |
| 2082 | GCUAUUCU G UGUUGGGG | 1637 | CCCCAACA GGAGGAAACUCC CU UCAAGGACAUCGUCCGGG AGAAUAGC | 10299 |
| 2084 | UAUUCUGU G UUGGGGUG | 1638 | CACCCCAA GGAGGAAACUCC CU UCAAGGACAUCGUCCGGG ACAGAAUA | 10300 |
| 2167 | UCAGCUAU G UCAACGUU | 1639 | AACGUUGA GGAGGAAACUCC CU UCAAGGACAUCGUCCGGG AUAGCUGA | 10301 |
| 2205 | CAACUAUU G UGGUUUCA | 1640 | UGAAACCA GGAGGAAACUCC CU UCAAGGACAUCGUCCGGG AAUAGUUG | 10302 |
| 2222 | CAUUUCCU G UCUUACUU | 1641 | AAGUAAGA GGAGGAAACUCC CU UCAAGGACAUCGUCCGGG AGGAAAUG | 10303 |
| 2245 | GAGAAACU G UUCUUGAA | 1642 | UUCAAGAA GGAGGAAACUCC CU UCAAGGACAUCGUCCGGG AGUUUCUC | 10304 |
| 2262 | UAUUUGGU G UCUUUUGG | 1643 | CCAAAAGA GGAGGAAACUCC CU UCAAGGACAUCGUCCGGG ACCAAAUA | 10305 |
| 2274 | UUUGGAGU G UGGAUUCG | 1644 | CGAAUCCA GGAGGAAACUCC CU UCAAGGACAUCGUCCGGG ACUCCAAA | 10306 |
| 2344 | AAACUACU G UUGUUAGA | 1645 | UCUAACAA GGAGGAAACUCC CU UCAAGGACAUCGUCCGGG AGUAGUUU | 10307 |
| 2347 | CUACUGUU G UUAGACGA | 1646 | UCGUCUAA GGAGGAAACUCC CU UCAAGGACAUCGUCCGGG AACAGUAG | 10308 |
| 2450 | AUCUCAAU G UUAGUAUU | 1647 | AAUACUAA GGAGGAAACUCC CU UCAAGGACAUCGUCCGGG AUUGAGAU | 10309 |
| 2573 | AGGACAUU G UUGAUAGA | 1648 | UCUAUCAA GGAGGAAACUCC CU UCAAGGACAUCGUCCGGG AAUGUCCU | 10310 |
| 2583 | UGAUAGAU G UAAGCAAU | 1649 | AUUGCUUA GGAGGAAACUCC CU UCAAGGACAUCGUCCGGG AUCUAUCA | 10311 |
| 2594 | AGCAAUUU G UGGGGCCC | 1650 | GGGCCCCA GGAGGAAACUCC CU UCAAGGACAUCGUCCGGG AAAUUGCU | 10312 |
| 2663 | AUCCCAAU G UUACUAAA | 1651 | UUUAGUAA GGAGGAAACUCC CU UCAAGGACAUCGUCCGGG AUUGGGAU | 10313 |
| 2717 | CAGAGUAU G UAGUUAAU | 1652 | AUUAACUA GGAGGAAACUCC CU UCAAGGACAUCGUCCGGG AUACUCUG | 10314 |
| 2901 | AUCUUUCU G UCCCCAAU | 1653 | AUUGGGGA GGAGGAAACUCC CU UCAAGGACAUCGUCCGGG AGAAAGAU | 10315 |
| 3071 | GGGGGACU G UUGGGGUG | 1654 | CACCCCAA GGAGGAAACUCC CU UCAAGGACAUCGUCCGGG AGUCCCCC | 10316 |
| 3111 | UCACAACU G UGCCAGCA | 1655 | UGCUGGCA GGAGGAAACUCC CU UCAAGGACAUCGUCCGGG AGUUGUGA | 10317 |
| 40 | AUCCCAGA G UCAGGGCC | 1656 | GGCCCUGA GGAGGAAACUCC CU UCAAGGACAUCGUCCGGG UCUGGGAU | 10318 |
| 46 | GAGUCAGG G CCCUGUAC | 1657 | GUACAGGG GGAGGAAACUCC CU UCAAGGACAUCGUCCGGG CCUGACUC | 10319 |
| 65 | UCCUGCUG G UGGCUCCA | 1658 | UGGAGCCA GGAGGAAACUCC CU UCAAGGACAUCGUCCGGG CAGCAGGA | 10320 |
| 68 | UGCUGGUG G CUCCAGUU | 1659 | AACUGGAG GGAGGAAACUCC CU UCAAGGACAUCGUCCGGG CACCAGCA | 10321 |
| 74 | UGGCUCCA G UUCAGGAA | 1660 | UUCCUGAA GGAGGAAACUCC CU UCAAGGACAUCGUCCGGG UGGAGCCA | 10322 |
| 85 | CAGGAACA G UGAGCCCU | 1661 | AGGGCUCA GGAGGAAACUCC CU UCAAGGACAUCGUCCGGG UGUUCCUG | 10323 |
| 89 | AACAGUGA G CCCUGCUC | 1662 | GAGCAGGG GGAGGAAACUCC CU UCAAGGACAUCGUCCGGG UCACUGUU | 10324 |
| 120 | GCCAUAUC G UCAAUCUU | 1663 | AAGAUUGA GGAGGAAACUCC CU UCAAGGACAUCGUCCGGG GAUAUGGC | 10325 |
| 196 | CCCUGCUC G UGUUACAG | 1664 | CUGUAACA GGAGGAAACUCC CU UCAAGGACAUCGUCCGGG GAGCAGGG | 10326 |
| 205 | UGUUACAG G CGGGGUUU | 1665 | AAACCCCG GGAGGAAACUCC CU UCAAGGACAUCGUCCGGG CUGUAACA | 10327 |
| 210 | CAGGCGGG G UUUUUCUU | 1666 | AAGAAAAA GGAGGAAACUCC CU UCAAGGACAUCGUCCGGG CCCGCCUG | 10328 |
| 248 | ACCACAGA G UCUAGACU | 1667 | AGUCUAGA GGAGGAAACUCC CU UCAAGGACAUCGUCCGGG UCUGUGGU | 10329 |
| 258 | CUAGACUC G UGGUGGAC | 1668 | GUCCACCA GGAGGAAACUCC CU UCAAGGACAUCGUCCGGG GAGUCUAG | 10330 |
| 261 | GACUCGUG G UGGACUUC | 1669 | GAAGUCCA GGAGGAAACUCC CU UCAAGGACAUCGUCCGGG CACGAGUC | 10331 |
| 295 | GAACACCC G UGUGUCUU | 1670 | AAGACACA GGAGGAAACUCC CU UCAAGGACAUCGUCCGGG GGGUGUUC | 10332 |
| 305 | GUGUCUUG G CCAAAAUU | 1671 | AAUUUUGG GGAGGAAACUCC CU UCAAGGACAUCGUCCGGG CAAGACAC | 10333 |
| 318 | AAUUCGCA G UCCCAAAU | 1672 | AUUUGGGA GGAGGAAACUCC CU UCAAGGACAUCGUCCGGG UGCGAAUU | 10334 |
| 332 | AAUCUCCA G UCACUCAC | 1673 | GUGAGUGA GGAGGAAACUCC CU UCAAGGACAUCGUCCGGG UGGAGAUU | 10335 |
| 368 | UUGUCCUG G UUAUCGCU | 1674 | AGCGAUAA GGAGGAAACUCC CU UCAAGGACAUCGUCCGGG CAGGACAA | 10336 |
| 390 | UGUCUGCG G CGUUUUAU | 1675 | AUAAAACG GGAGGAAACUCC CU UCAAGGACAUCGUCCGGG CGCAGACA | 10337 |
| 392 | UCUGCGGC G UUUUAUCA | 1676 | UGAUAAAA GGAGGAAACUCC CU UCAAGGACAUCGUCCGGG GCCGCAGA | 10338 |
| 442 | UCUUGUUG G UUCUUCUG | 1677 | CAGAAGAA GGAGGAAACUCC CU UCAAGGACAUCGUCCGGG CAACAAGA | 10339 |
| 461 | CUAUCAAG G UAUGUUGC | 1678 | GCAACAUA GGAGGAAACUCC CU UCAAGGACAUCGUCCGGG CUUGAUAG | 10340 |
| 472 | UGUUGCCC G UUUGUCCU | 1679 | AGGACAAA GGAGGAAACUCC CU UCAAGGACAUCGUCCGGG GGGCAACA | 10341 |
| 506 | AACAACCA G CACCGGAC | 1680 | GUCCGGUG GGAGGAAACUCC CU UCAAGGACAUCGUCCGGG UGGUUGUU | 10342 |
| 625 | CAUCUUGG G CUUUCGCA | 1681 | UGCGAAAG GGAGGAAACUCC CU UCAAGGACAUCGUCCGGG CCAAGAUG | 10343 |
| 648 | CUAUGGGA G UGGGCCUC | 1682 | GAGGCCCA GGAGGAAACUCC CU UCAAGGACAUCGUCCGGG UCCCAUAG | 10344 |
| 652 | GGGAGUGG G CCUCAGUC | 1683 | GACUGAGG GGAGGAAACUCC CU UCAAGGACAUCGUCCGGG CCACUCCC | 10345 |
| 658 | GGGCCUCA G UCCGUUUC | 1684 | GAAACGGA GGAGGAAACUCC CU UCAAGGACAUCGUCCGGG UGAGGCCC | 10346 |
| 662 | CUCAGUCC G UUUCUCUU | 1685 | AAGAGAAA GGAGGAAACUCC CU UCAAGGACAUCGUCCGGG GGACUGAG | 10347 |
| 672 | UUCUCUUG G CUCAGUUU | 1686 | AAACUGAG GGAGGAAACUCC CU UCAAGGACAUCGUCCGGG CAAGAGAA | 10348 |
| 677 | UUGGCUCA G UUUACUAG | 1687 | CUAGUAAA GGAGGAAACUCC CU UCAAGGACAUCGUCCGGG UGAGCCAA | 10349 |
| 685 | GUUUACUA G UGCCAUUU | 1688 | AAAUGGCA GGAGGAAACUCC CU UCAAGGACAUCGUCCGGG UAGUAAAC | 10350 |
| 699 | UUUGUUCA G UGGUUCGU | 1689 | ACGAACCA GGAGGAAACUCC CU UCAAGGACAUCGUCCGGG UGAACAAA | 10351 |
| 702 | GUUCAGUG G UUCGUAGG | 1690 | CCUACGAA GGAGGAAACUCC CU UCAAGGACAUCGUCCGGG CACUGAAC | 10352 |
| 706 | AGUGGUUC G UAGGGCUU | 1691 | AAGCCCUA GGAGGAAACUCC CU UCAAGGACAUCGUCCGGG GAACCACU | 10353 |
| 711 | UUCGUAGG G CUUUCCCC | 1692 | GGGGAAAG GGAGGAAACUCC CU UCAAGGACAUCGUCCGGG CCUACGAA | 10354 |
| 729 | ACUGUCUG G CUUUCAGU | 1693 | ACUGAAAG GGAGGAAACUCC CU UCAAGGACAUCGUCCGGG CAGACAGU | 10355 |
| 736 | GGCUUUCA G UUAUAUGG | 1694 | CCAUAUAA GGAGGAAACUCC CU UCAAGGACAUCGUCCGGG UGAAAGCC | 10356 |
| 753 | AUGAUGUG G UUUUGGGG | 1695 | CCCCAAAA GGAGGAAACUCC CU UCAAGGACAUCGUCCGGG CACAUCAU | 10357 |
| 762 | UUUUGGGG G CCAAGUCU | 1696 | AGACUUGG GGAGGAAACUCC CU UCAAGGACAUCGUCCGGG CCCCAAAA | 10358 |
| 767 | GGGGCCAA G UGUGUACA | 1697 | UGUACAGA GGAGGAAACUCC CU UCAAGGACAUCGUCCGGG UUGGCCCC | 10359 |
| 785 | CAUCUUGA G UCCCUUUA | 1698 | UAAAGGGA GGAGGAAACUCC CU UCAAGGACAUCGUCCGGG UCAAGAUG | 10360 |
| 826 | GUCUUUGG G UAUACAUU | 1699 | AAUGUAUA GGAGGAAACUCC CU UCAAGGACAUCGUCCGGG CCAAAGAC | 10361 |
| 898 | AAUUGGGA G UUGGGGCA | 1700 | UGCCCCAA GGAGGAAACUCC CU UCAAGGACAUCGUCCGGG UCCCAAUU | 10362 |
| 904 | GAGUUGGG G CACAUUGC | 1701 | GCAAUGUG GGAGGAAACUCC CU UCAAGGACAUCGUCCGGG CCCAACUC | 10363 |
| 971 | GUAAACAG G CCUAUUGA | 1702 | UCAAUAGG GGAGGAAACUCC CU UCAAGGACAUCGUCCGGG CUGUUUAC | 10364 |
| 987 | AUUGGAAA G UAUGUCAA | 1703 | UUGACAUA GGAGGAAACUCC CU UCAAGGACAUCGUCCGGG UUUCCAAU | 10365 |
| 1006 | AAUUGUGG G UCUUUUGG | 1704 | CCAAAAGA GGAGGAAACUCC CU UCAAGGACAUCGUCCGGG CCACAAUU | 10366 |
| 1016 | CUUUUGGG G UUUGCCGC | 1705 | GCGGCAAA GGAGGAAACUCC CU UCAAGGACAUCGUCCGGG CCCAAAAG | 10367 |
| 1080 | GCAUACAA G CAAAACAG | 1706 | CUGUUUUG GGAGGAAACUCC CU UCAAGGACAUCGUCCGGG UUGUAUGC | 10368 |
| 1089 | CAAAACAG G CUUUUACU | 1707 | AGUAAAAG GGAGGAAACUCC CU UCAAGGACAUCGUCCGGG CUGUUUUG | 10369 |
| 1116 | CUUACAAG G CCUUUCUA | 1708 | UAGAAAGG GGAGGAAACUCC CU UCAAGGACAUCGUCCGGG CUUGUAAG | 10370 |
| 1126 | CUUUCUAA G UAAACAGU | 1709 | ACUGUUUA GGAGGAAACUCC CU UCAAGGACAUCGUCCGGG UUAGAAAG | 10371 |
| 1133 | AGUAAACA G UAUGUGAA | 1710 | UUCACAUA GGAGGAAACUCC CU UCAAGGACAUCGUCCGGG UGUUUACU | 10372 |
| 1152 | UUUACCCC G UUGCUCGG | 1711 | CCGAGCAA GGAGGAAACUCC CU UCAAGGACAUCGUCCGGG GGGGUAAA | 10373 |
| 1160 | GUUGCUCG G CAACGGCC | 1712 | GGCCGUUG GGAGGAAACUCC CU UCAAGGACAUCGUCCGGG CGAGCAAC | 10374 |
| 1166 | CGGCAACG G CCUGGUCU | 1713 | AGACCAGG GGAGGAAACUCC CU UCAAGGACAUCGUCCGGG CGUUGCCG | 10375 |
| 1171 | ACGGCCUG G UCUAUGCC | 1714 | GGCAUAGA GGAGGAAACUCC CU UCAAGGACAUCGUCCGGG CAGGCCGU | 10376 |
| 1182 | UAUGCCAA G UGUUUGCU | 1715 | AGCAAACA GGAGGAAACUCC CU UCAAGGACAUCGUCCGGG UUGGCAUA | 10377 |
| 1207 | CCCCACUG G UUGGGGCU | 1716 | AGCCCCAA GGAGGAAACUCC CU UCAAGGACAUCGUCCGGG CAGUGGGG | 10378 |
| 1213 | UGGUUGGG G CUUGGCCA | 1717 | UGGCCAAG GGAGGAAACUCC CU UCAAGGACAUCGUCCGGG CCCAACCA | 10379 |
| 1218 | GGGGCUUG G CCAUAGGC | 1718 | GCCUAUGG GGAGGAAACUCC CU UCAAGGACAUCGUCCGGG CAAGCCCC | 10380 |
| 1225 | GGCCAUAG G CCAUCAGC | 1719 | GCUGAUGG GGAGGAAACUCC CU UCAAGGACAUCGUCCGGG CUAUGGCC | 10381 |
| 1232 | GGCCAUCA G CGCAUGCG | 1720 | CGCAUGCG GGAGGAAACUCC CU UCAAGGACAUCGUCCGGG UGAUGGCC | 10382 |
| 1240 | GCGCAUGC G UGGAACCU | 1721 | AGGUUCCA GGAGGAAACUCC CU UCAAGGACAUCGUCCGGG GCAUGCGC | 10383 |
| 1287 | AACUCCUA G CCGCUUGU | 1722 | ACAAGCGG GGAGGAAACUCC CU UCAAGGACAUCGUCCGGG UAGGAGUU | 10384 |
| 1306 | UGCUCGCA G CAGGUCUG | 1723 | CAGACCUG GGAGGAAACUCC CU UCAAGGACAUCGUCCGGG UGCGAGCA | 10385 |
| 1310 | CGCAGCAG G UCUGGGGC | 1724 | GCCCCAGA GGAGGAAACUCC CU UCAAGGACAUCGUCCGGG CUGCUGCG | 10386 |
| 1317 | GGUCUGGG G CAAAACUC | 1725 | GAGUUUUG GGAGGAAACUCC CU UCAAGGACAUCGUCCGGG CCCAGACC | 10387 |
| 1347 | AUUCUGUC G UGCUCUCC | 1726 | GGAGAGCA GGAGGAAACUCC CU UCAAGGACAUCGUCCGGG GACAGAAU | 10388 |
| 1379 | UUUCCAUG G CUGCUAGG | 1727 | CCUAGCAG GGAGGAAACUCC CU UCAAGGACAUCGUCCGGG CAUGGAAA | 10389 |
| 1387 | GCUGCUAG G CUGUGCUG | 1728 | CAGCACAG GGAGGAAACUCC CU UCAAGGACAUCGUCCGGG CUAGCAGC | 10390 |
| 1418 | CGCGGGAC G UCCUUUGU | 1729 | ACAAAGGA GGAGGAAACUCC CU UCAAGGACAUCGUCCGGG GUCCCGCG | 10391 |
| 1431 | UUGUUUAC G UCCCGUCG | 1730 | CGACGGGA GGAGGAAACUCC CU UCAAGGACAUCGUCCGGG GUAAACAA | 10392 |
| 1436 | UACGUCCC G UCGGCGCU | 1731 | AGCGCCGA GGAGGAAACUCC CU UCAAGGACAUCGUCCGGG GGGACGUA | 10393 |
| 1440 | UCCCGUCG G CGCUGAAU | 1732 | AUUCAGCG GGAGGAAACUCC CU UCAAGGACAUCGUCCGGG CGACGGGA | 10394 |
| 1471 | CUCCCGGG G CCGCUUGG | 1733 | CCAAGCGG GGAGGAAACUCC CU UCAAGGACAUCGUCCGGG CCCGGGAG | 10395 |
| 1481 | CGCUUGGG G CUCUACCG | 1734 | CGGUAGAG GGAGGAAACUCC CU UCAAGGACAUCGUCCGGG CCCAAGCG | 10396 |
| 1517 | UACCGACC G UCCACGGG | 1735 | CCCGUGGA GGAGGAAACUCC CU UCAAGGACAUCGUCCGGG GGUCGGUA | 10397 |
| 1526 | UCCACGGG G CGCACCUC | 1736 | GAGGUGCG GGAGGAAACUCC CU UCAAGGACAUCGUCCGGG CCCGUGGA | 10398 |
| 1553 | GACUCCCC G UCUGUGCC | 1737 | GGCACAGA GGAGGAAACUCC CU UCAAGGACAUCGUCCGGG GGGGAGUC | 10399 |
| 1579 | GCCGGACC G UGUGCACU | 1738 | AGUGCACA GGAGGAAACUCC CU UCAAGGACAUCGUCCGGG GGUCCGGC | 10400 |
| 1605 | CUCUGCAC G UCGCAUGG | 1739 | CCAUGCGA GGAGGAAACUCC CU UCAAGGACAUCGUCCGGG GUGCAGAG | 10401 |
| 1622 | AGACCACC G UGAACGCC | 1740 | GGCGUUCA GGAGGAAACUCC CU UCAAGGACAUCGUCCGGG GGUGGUCU | 10402 |
| 1649 | UGCCCAAG G UCUUGCAU | 1741 | AUGCAAGA GGAGGAAACUCC CU UCAAGGACAUCGUCCGGG CUUGGGCA | 10403 |
| 1679 | GACUUUCA G CAAUGUCA | 1742 | UGACAUUG GGAGGAAACUCC CU UCAAGGACAUCGUCCGGG UGAAAGUC | 10404 |
| 1703 | ACCUUGAG G CAUACUUC | 1743 | GAAGUAUG GGAGGAAACUCC CU UCAAGGACAUCGUCCGGG CUCAAGGU | 10405 |
| 1732 | UUUAAUGA G UGGGAGGA | 1744 | UCCUCCCA GGAGGAAACUCC CU UCAAGGACAUCGUCCGGG UCAUUAAA | 10406 |
| 1741 | UGGGAGGA G UUGGGGGA | 1745 | UCCCCCAA GGAGGAAACUCC CU UCAAGGACAUCGUCCGGG UCCUCCCA | 10407 |
| 1754 | GGGAGGAG G UUAGGUUA | 1746 | UAACCUAA GGAGGAAACUCC CU UCAAGGACAUCGUCCGGG CUCCUCCC | 10408 |
| 1759 | GAGGUUAG G UUAAAGGU | 1747 | ACCUUUAA GGAGGAAACUCC CU UCAAGGACAUCGUCCGGG CUAACCUC | 10409 |
| 1766 | GGUUAAAG G UCUUUGUA | 1748 | UACAAAGA GGAGGAAACUCC CU UCAAGGACAUCGUCCGGG CUUUAACC | 10410 |
| 1782 | ACUAGGAG G CUGUAGGC | 1749 | GCCUACAG GGAGGAAACUCC CU UCAAGGACAUCGUCCGGG CUCCUAGU | 10411 |
| 1789 | GGCUGUAG G CAUAAAUU | 1750 | AAUUUAUG GGAGGAAACUCC CU UCAAGGACAUCGUCCGGG CUACAGCC | 10412 |
| 1799 | AUAAAUUG G UGUGUUCA | 1751 | UGAACACA GGAGGAAACUCC CU UCAAGGACAUCGUCCGGG CAAUUUAU | 10413 |
| 1811 | GUUCACCA G CACCAUGC | 1752 | GCAUGGUG GGAGGAAACUCC CU UCAAGGACAUCGUCCGGG UGGUGAAC | 10414 |
| 1870 | CUGUUCAA G CCUCCAAG | 1753 | CUUGGAGG GGAGGAAACUCC CU UCAAGGACAUCGUCCGGG UUGAACAG | 10415 |
| 1878 | GCCUCCAA G CUGUGCCU | 1754 | AGGCACAG GGAGGAAACUCC CU UCAAGGACAUCGUCCGGG UUGGAGGC | 10416 |
| 1890 | UGCCUUGG G UGGCUUUG | 1755 | CAAAGCCA GGAGGAAACUCC CU UCAAGGACAUCGUCCGGG CCAAGGCA | 10417 |
| 1893 | CUUGGGUG G CUUUGGGG | 1756 | CCCCAAAG GGAGGAAACUCC CU UCAAGGACAUCGUCCGGG CACCCAAG | 10418 |
| 1901 | GCUUUGGG G CAUGGACA | 1757 | UGUCCAUG GGAGGAAACUCC CU UCAAGGACAUCGUCCGGG CCCAAAGC | 10419 |
| 1917 | AUUGACCC G UAUAAAGA | 1758 | UCUUUAUA GGAGGAAACUCC CU UCAAGGACAUCGUCCGGG GGGUCAAU | 10420 |
| 1933 | AAUUUGGA G CUUCUGUG | 1759 | CACAGAAG GGAGGAAACUCC CU UCAAGGACAUCGUCCGGG UCCAAAUU | 10421 |
| 1944 | UCUGUGGA G UUACUCUC | 1760 | GAGAGUAA GGAGGAAACUCC CU UCAAGGACAUCGUCCGGG UCCACAGA | 10422 |
| 2023 | AUCGGGGG G CCUUAGAG | 1761 | CUCUAAGG GGAGGAAACUCC CU UCAAGGACAUCGUCCGGG CCCCCGAU | 10423 |
| 2031 | GCCUUAGA G UCUCCGGA | 1762 | UCCGGAGA GGAGGAAACUCC CU UCAAGGACAUCGUCCGGG UCUAAGGC | 10424 |
| 2062 | ACCAUACG G CACUCAGG | 1763 | CCUGAGUG GGAGGAAACUCC CU UCAAGGACAUCGUCCGGG CGUAUGGU | 10425 |
| 2070 | GCACUCAG G CAAGCUAU | 1764 | AUAGCUUG GGAGGAAACUCC CU UCAAGGACAUCGUCCGGG CUGAGUGC | 10426 |
| 2074 | UCAGGCAA G CUAUUCUG | 1765 | CAGAAUAG GGAGGAAACUCC CU UCAAGGACAUCGUCCGGG UUGCCUGA | 10427 |
| 2090 | GUGUUGGG G UGAGUUGA | 1766 | UCAACUCA GGAGGAAACUCC CU UCAAGGACAUCGUCCGGG CCCAACAC | 10428 |
| 2094 | UGGGGUGA G UUGAUGAA | 1767 | UUCAUCAA GGAGGAAACUCC CU UCAAGGACAUCGUCCGGG UCACCCCA | 10429 |
| 2107 | UGAAUCUA G CCACCUGG | 1768 | CCAGGUGG GGAGGAAACUCC CU UCAAGGACAUCGUCCGGG UAGAUUCA | 10430 |
| 2116 | CCACCUGG G UGGGAAGU | 1769 | ACUUCCCA GGAGGAAACUCC CU UCAAGGACAUCGUCCGGG CCAGGUGG | 10431 |
| 2123 | GGUGGGAA G UAAUUUGG | 1770 | CCAAAUUA GGAGGAAACUCC CU UCAAGGACAUCGUCCGGG UUCCCACC | 10432 |
| 2140 | AAGAUCCA G CAUCCAGG | 1771 | CCUGGAUG GGAGGAAACUCC CU UCAAGGACAUCGUCCGGG UGGAUCUU | 10433 |
| 2155 | GGGAAUUA G UAGUCAGC | 1772 | GCUGACUA GGAGGAAACUCC CU UCAAGGACAUCGUCCGGG UAAUUCCC | 10434 |
| 2158 | AAUUAGUA G UCAGCUAU | 1773 | AUAGCUGA GGAGGAAACUCC CU UCAAGGACAUCGUCCGGG UACUAAUU | 10435 |
| 2162 | AGUAGUCA G CUAUGUCA | 1774 | UGACAUAG GGAGGAAACUCC CU UCAAGGACAUCGUCCGGG UGACUACU | 10436 |
| 2173 | AUGUCAAC G UUAAUAUG | 1775 | CAUAUUAA GGAGGAAACUCC CU UCAAGGACAUCGUCCGGG GUUGACAU | 10437 |
| 2183 | UAAUAUGG G CCUAAAAA | 1776 | UUUUUAGG GGAGGAAACUCC CU UCAAGGACAUCGUCCGGG CCAUAUUA | 10438 |
| 2208 | CUAUUGUG G UUUCACAU | 1777 | AUGUGAAA GGAGGAAACUCC CU UCAAGGACAUCGUCCGGG CACAAUAG | 10439 |
| 2235 | ACUUUUGG G CGAGAAAC | 1778 | GUUUCUCG GGAGGAAACUCC CU UCAAGGACAUCGUCCGGG CCAAAAGU | 10440 |
| 2260 | AAUAUUUG G UGUCUUUU | 1779 | AAAAGACA GGAGGAAACUCC CU UCAAGGACAUCGUCCGGG CAAAUAUU | 10441 |
| 2272 | CUUUUGGA G UGUGGAUU | 1780 | AAUCCACA GGAGGAAACUCC CU UCAAGGACAUCGUCCGGG UCCAAAAG | 10442 |
| 2360 | ACGAAGAG G CAGGUCCC | 1781 | GGGACCUG GGAGGAAACUCC CU UCAAGGACAUCGUCCGGG CUCUUCGU | 10443 |
| 2364 | AGAGGCAG G UCCCCUAG | 1782 | CUAGGGGA GGAGGAAACUCC CU UCAAGGACAUCGUCCGGG CUGCCUCU | 10444 |
| 2403 | AGACGAAG G UCUCAAUC | 1783 | GAUUGAGA GGAGGAAACUCC CU UCAAGGACAUCGUCCGGG CUUCGUCU | 10445 |
| 2417 | AUCGCCGC G UCGCAGAA | 1784 | UUCUGCGA GGAGGAAACUCC CU UCAAGGACAUCGUCCGGG GCGGCGAU | 10446 |
| 2454 | CAAUGUUA G UAUUCCUU | 1785 | AAGGAAUA GGAGGAAACUCC CU UCAAGGACAUCGUCCGGG UAACAUUG | 10447 |
| 2474 | CACAUAAG G UGGGAAAC | 1786 | GUUUCCCA GGAGGAAACUCC CU UCAAGGACAUCGUCCGGG CUUAUGUG | 10448 |
| 2491 | UUUACGGG G CUUUAUUC | 1787 | GAAUAAAG GGAGGAAACUCC CU UCAAGGACAUCGUCCGGG CCCGUAAA | 10449 |
| 2507 | CUUCUACG G UACCUUGC | 1788 | GCAAGGUA GGAGGAAACUCC CU UCAAGGACAUCGUCCGGG CGUAGAAG | 10450 |
| 2530 | CCUAAAUG G CAAACUCC | 1789 | GGAGUUUG GGAGGAAACUCC CU UCAAGGACAUCGUCCGGG CAUUUAGG | 10451 |
| 2587 | AGAUGUAA G CAAUUUGU | 1790 | ACAAAUUG GGAGGAAACUCC CU UCAAGGACAUCGUCCGGG UUACAUCU | 10452 |
| 2599 | UUUGUGGG G CCCCUUAC | 1791 | GUAAGGGG GGAGGAAACUCC CU UCAAGGACAUCGUCCGGG CCCACAAA | 10453 |
| 2609 | CCCUUACA G UAAAUGAA | 1792 | UUCAUUUA GGAGGAAACUCC CU UCAAGGACAUCGUCCGGG UGUAAGGG | 10454 |
| 2650 | CCUGCUAG G UUUUAUCC | 1793 | GGAUAAAA GGAGGAAACUCC CU UCAAGGACAUCGUCCGGG CUAGCAGG | 10455 |
| 2701 | AUCAAACC G UAUUAUCC | 1794 | GGAUAAUA GGAGGAAACUCC CU UCAAGGACAUCGUCCGGG GGUUUGAU | 10456 |
| 2713 | UAUCCAGA G UAUGUAGU | 1795 | ACUACAUA GGAGGAAACUCC CU UCAAGGACAUCGUCCGGG UCUGGAUA | 10457 |
| 2720 | AGUAUGUA G UUAAUCAU | 1796 | AUGAUUAA GGAGGAAACUCC CU UCAAGGACAUCGUCCGGG UACAUACU | 10458 |
| 2768 | UUUGGAAG G CGGGGAUC | 1797 | GAUCCCCG GGAGGAAACUCC CU UCAAGGACAUCGUCCGGG CUUCCAAA | 10459 |
| 2791 | AAAAGAGA G UCCACACG | 1798 | CGUGUGGA GGAGGAAACUCC CU UCAAGGACAUCGUCCGGG UCUCUUUU | 10460 |
| 2799 | GUCCACAC G UAGCGCCU | 1799 | AGGCGCUA GGAGGAAACUCC CU UCAAGGACAUCGUCCGGG GUGUGGAC | 10461 |
| 2802 | CACACGUA G CGCCUCAU | 1800 | AUGAGGCG GGAGGAAACUCC CU UCAAGGACAUCGUCCGGG UACGUGUG | 10462 |
| 2818 | UUUUGCGG G UCACCAUA | 1801 | UAUGGUGA GGAGGAAACUCC CU UCAAGGACAUCGUCCGGG CCGCAAAA | 10463 |
| 2848 | GAUCUACA G CAUGGGAG | 1802 | CUCCCAUG GGAGGAAACUCC CU UCAAGGACAUCGUCCGGG UGUAGAUC | 10464 |
| 2857 | CAUGGGAG G UUGGUCUU | 1803 | AAGACCAA GGAGGAAACUCC CU UCAAGGACAUCGUCCGGG CUCCCAUG | 10465 |
| 2861 | GGAGGUUG G UCUUCCAA | 1804 | UUGGAAGA GGAGGAAACUCC CU UCAAGGACAUCGUCCGGG CAACCUCC | 10466 |
| 2881 | UCGAAAAG G CAUGGGGA | 1805 | UCCCCAUG GGAGGAAACUCC CU UCAAGGACAUCGUCCGGG CUUUUCGA | 10467 |
| 2936 | GAUCAUCA G UUGGACCC | 1806 | GGGUCCAA GGAGGAAACUCC CU UCAAGGACAUCGUCCGGG UGAUGAUC | 10468 |
| 2955 | CAUUCAAA G CCAACUCA | 1807 | UGAGUUGG GGAGGAAACUCC CU UCAAGGACAUCGUCCGGG UUUGAAUG | 10469 |
| 2964 | CCAACUCA G UAAAUCCA | 1808 | UGGAUUUA GGAGGAAACUCC CU UCAAGGACAUCGUCCGGG UGAGUUGG | 10470 |
| 3005 | GACAACUG G CCGGACGC | 1809 | GCGUCCGG GGAGGAAACUCC CU UCAAGGACAUCGUCCGGG CAGUUGUC | 10471 |
| 3021 | CCAACAAG G UGGGAGUG | 1810 | CACUCCCA GGAGGAAACUCC CU UCAAGGACAUCGUCCGGG CUUGUUGG | 10472 |
| 3027 | AGGUGGGA G UGGGAGCA | 1811 | UGCUCCCA GGAGGAAACUCC CU UCAAGGACAUCGUCCGGG UCCCACCU | 10473 |
| 3033 | GAGUGGGA G CAUUCGGG | 1812 | CCCGAAUG GGAGGAAACUCC CU UCAAGGACAUCGUCCGGG UCCCACUC | 10474 |
| 3041 | GCAUUCGG G CCAGGGUU | 1813 | AACCCUGG GGAGGAAACUCC CU UCAAGGACAUCGUCCGGG CCGAAUGC | 10475 |
| 3047 | GGGCCAGG G UUCACCCC | 1814 | GGGGUGAA GGAGGAAACUCC CU UCAAGGACAUCGUCCGGG CCUGGCCC | 10476 |
| 3077 | CUGUUGGG G UGGAGCCC | 1815 | GGGCUCCA GGAGGAAACUCC CU UCAAGGACAUCGUCCGGG CCCAACAG | 10477 |
| 3082 | GGGGUGGA G CCCUCACG | 1816 | CGUGAGGG GGAGGAAACUCC CU UCAAGGACAUCGUCCGGG UCCACCCC | 10478 |
| 3097 | CGCUCAGG G CCUACUCA | 1817 | UGAGUAGG GGAGGAAACUCC CU UCAAGGACAUCGUCCGGG CCUGAGCG | 10479 |
| 3117 | CUGUGCCA G CAGCUCCU | 1818 | AGGAGCUG GGAGGAAACUCC CU UCAAGGACAUCGUCCGGG UGGCACAG | 10480 |
| 3120 | UGCCAGCA G CUCCUCCU | 1819 | AGGAGGAG GGAGGAAACUCC CU UCAAGGACAUCGUCCGGG UGCUGGCA | 10481 |
| 3146 | ACCAAUCG G CAGUCAGG | 1820 | CCUGACUG GGAGGAAACUCC CU UCAAGGACAUCGUCCGGG CGAUUGGU | 10482 |
| 3149 | AAUCGGCA G UCAGGAAG | 1821 | CUUCCUGA GGAGGAAACUCC CU UCAAGGACAUCGUCCGGG UGCCGAUU | 10483 |
| 3158 | UCAGGAAG G CAGCCUAC | 1822 | GUAGGCUG GGAGGAAACUCC CU UCAAGGACAUCGUCCGGG CUUCCUGA | 10484 |
| 3161 | GGAAGGCA G CCUACUCC | 1823 | GGAGUAGG GGAGGAAACUCC CU UCAAGGACAUCGUCCGGG UGCCUUCC | 10485 |
| 3204 | AUCCUCAG G CCAUGCAG | 1824 | CUGCAUGG GGAGGAAACUCC CU UCAAGGACAUCGUCCGGG CUGAGGAU | 10486 |
| 31 | CUCUUCAA G AUCCCAGA | 1999 | UCUGGGAU GGAGGAAACUCC CU UCAAGGACAUCGUCCGGG UUGAAGAG | 10487 |
| 38 | AGAUCCCA G AGUCAGGG | 2000 | CCCUGACU GGAGGAAACUCC CU UCAAGGACAUCGUCCGGG UGGGAUCU | 10488 |
| 44 | CAGAGUCA G GGCCCUGU | 2001 | ACAGGGCC GGAGGAAACUCC CU UCAAGGACAUCGUCCGGG UGACUCUG | 10489 |
| 45 | AGAGUCAG G GCCCUGUA | 2002 | UACAGGGC GGAGGAAACUCC CU UCAAGGACAUCGUCCGGG CUGACUCU | 10490 |
| 64 | UUCCUGCU G GUGGCUCC | 2003 | GGAGCCAC GGAGGAAACUCC CU UCAAGGACAUCGUCCGGG AGCAGGAA | 10491 |
| 67 | CUGCUGGU G GCUCCAGU | 2004 | ACUGGAGC GGAGGAAACUCC CU UCAAGGACAUCGUCCGGG ACCAGCAG | 10492 |
| 79 | CCAGUUCA G GAACAGUG | 2005 | CACUGUUC GGAGGAAACUCC CU UCAAGGACAUCGUCCGGG UGAACUGG | 10493 |
| 80 | CAGUUCAG G AACAGUGA | 2006 | UCACUGUU GGAGGAAACUCC CU UCAAGGACAUCGUCCGGG CUGAACUG | 10494 |
| 99 | CCUGCUCA G AAUACUGU | 2007 | ACAGUAUU GGAGGAAACUCC CU UCAAGGACAUCGUCCGGG UGAGCAGG | 10495 |
| 135 | UUAUCGAA G ACUGGGGA | 2008 | UCCCCAGU GGAGGAAACUCC CU UCAAGGACAUCGUCCGGG UUCGAUAA | 10496 |
| 139 | CGAAGACU G GGGACCCU | 2009 | AGGGUCCC GGAGGAAACUCC CU UCAAGGACAUCGUCCGGG AGUCUUCG | 10497 |
| 140 | GAAGACUG G GGACCCUG | 2010 | CAGGGUCC GGAGGAAACUCC CU UCAAGGACAUCGUCCGGG CAGUCUUC | 10498 |
| 141 | AAGACUGG G GACCCUGU | 2011 | ACAGGGUC GGAGGAAACUCC CU UCAAGGACAUCGUCCGGG CCAGUCUU | 10499 |
| 142 | AGACUGGG G ACCCUGUA | 2012 | UACAGGGU GGAGGAAACUCC CU UCAAGGACAUCGUCCGGG CCCAGUCU | 10500 |
| 159 | CCGAACAU G GAGAACAU | 2013 | AUGUUCUC GGAGGAAACUCC CU UCAAGGACAUCGUCCGGG AUGUUCGG | 10501 |
| 160 | CGAACAUG G AGAACAUC | 2014 | GAUGUUCU GGAGGAAACUCC CU UCAAGGACAUCGUCCGGG CAUGUUCG | 10502 |
| 162 | AACAUGGA G AACAUCGC | 2015 | GCGAUGUU GGAGGAAACUCC CU UCAAGGACAUCGUCCGGG UCCAUGUU | 10503 |
| 175 | UCGCAUCA G GACUCCUA | 2016 | UAGGAGUC GGAGGAAACUCC CU UCAAGGACAUCGUCCGGG UGAUGCGA | 10504 |
| 176 | CGCAUCAG G ACUCCUAG | 2017 | CUAGGAGU GGAGGAAACUCC CU UCAAGGACAUCGUCCGGG CUGAUGCG | 10505 |
| 184 | GACUCCUA G GACCCCUG | 2018 | CAGGGGUC GGAGGAAACUCC CU UCAAGGACAUCGUCCGGG UAGGAGUC | 10506 |
| 185 | ACUCCUAG G ACCCCUGC | 2019 | GCAGGGGU GGAGGAAACUCC CU UCAAGGACAUCGUCCGGG CUAGGAGU | 10507 |
| 204 | GUGUUACA G GCGGGGUU | 2020 | AACCCCGC GGAGGAAACUCC CU UCAAGGACAUCGUCCGGG UGUAACAC | 10508 |
| 207 | UUACAGGC G GGGUUUUU | 2021 | AAAAACCC GGAGGAAACUCC CU UCAAGGACAUCGUCCGGG GCCUGUAA | 10509 |
| 208 | UACAGGCG G GGUUUUUC | 2022 | GAAAAACC GGAGGAAACUCC CU UCAAGGACAUCGUCCGGG CGCCUGUA | 10510 |
| 209 | ACAGGCGG G GUUUUUCU | 2023 | AGAAAAAC GGAGGAAACUCC CU UCAAGGACAUCGUCCGGG CCGCCUGU | 10511 |
| 246 | AUACCACA G AGUCUAGA | 2024 | UCUAGACU GGAGGAAACUCC CU UCAAGGACAUCGUCCGGG UGUGGUAU | 10512 |
| 253 | AGAGUCUA G ACUCGUGG | 2025 | CCACGAGU GGAGGAAACUCC CU UCAAGGACAUCGUCCGGG UAGACUCU | 10513 |
| 260 | AGACUCGU G GUGGACUU | 2026 | AAGUCCAC GGAGGAAACUCC CU UCAAGGACAUCGUCCGGG ACGAGUCU | 10514 |
| 263 | CUCGUGGU G GACUUCUC | 2027 | GAGAAGUC GGAGGAAACUCC CU UCAAGGACAUCGUCCGGG ACCACGAG | 10515 |
| 264 | UCGUGGUG G ACUUCUCU | 2028 | AGAGAAGU GGAGGAAACUCC CU UCAAGGACAUCGUCCGGG CACCACGA | 10516 |
| 283 | AUUUUCUA G GGGGAACA | 2029 | UGUUCCCC GGAGGAAACUCC CU UCAAGGACAUCGUCCGGG UAGAAAAU | 10517 |
| 284 | UUUUCUAG G GGGAACAC | 2030 | GUGUUCCC GGAGGAAACUCC CU UCAAGGACAUCGUCCGGG CUAGAAAA | 10518 |
| 285 | UUUCUAGG G GGAACACC | 2031 | GGUCUUCC GGAGGAAACUCC CU UCAAGGACAUCGUCCGGG CCUAGAAA | 10519 |
| 286 | UUCUAGGG G GAACACCC | 2032 | GGGUGUUC GGAGGAAACUCC CU UCAAGGACAUCGUCCGGG CCCUAGAA | 10520 |
| 287 | UCUAGGGG G AACACCCG | 2033 | CGGGUGUU GGAGGAAACUCC CU UCAAGGACAUCGUCCGGG CCCCUAGA | 10521 |
| 304 | UGUGUCUU G GCCAAAAU | 2034 | AUUUUGGC GGAGGAAACUCC CU UCAAGGACAUCGUCCGGG AAGACACA | 10522 |
| 367 | UUUGUCCU G GUUAUCGC | 2035 | GCGAUAAC GGAGGAAACUCC CU UCAAGGACAUCGUCCGGG AGGACAAA | 10523 |
| 377 | UUAUCGCU G GAUGUGUC | 2036 | GACACAUC GGAGGAAACUCC CU UCAAGGACAUCGUCCGGG AGCGAUAA | 10524 |
| 378 | UAUCGCUG G AUGUGUCU | 2037 | AGACACAU GGAGGAAACUCC CU UCAAGGACAUCGUCCGGG CAGCGAUA | 10525 |
| 389 | CUGUCUGC G GCGUUUUA | 2038 | UAAAACGC GGAGGAAACUCC CU UCAAGGACAUCGUCCGGG GCAGACAC | 10526 |
| 441 | UUCUUGUU G GUUCUUCU | 2039 | AGAAGAAC GGAGGAAACUCC CU UCAAGGACAUCGUCCGGG AACAAGAA | 10527 |
| 450 | GUUCUUCU G GACUAUCA | 2040 | UGAUAGUC GGAGGAAACUCC CU UCAAGGACAUCGUCCGGG AGAAGAAC | 10528 |
| 451 | UUCUUCUG G ACUAUCAA | 2041 | UUGAUAGU GGAGGAAACUCC CU UCAAGGACAUCGUCCGGG CAGAAGAA | 10529 |
| 460 | ACUAUCAA G GUAUGUUG | 2042 | CAACAUAC GGAGGAAACUCC CU UCAAGGACAUCGUCCGGG UUGAUAGU | 10530 |
| 490 | UAAUUCCA G GAUCAUCA | 2043 | UGAUGAUC GGAGGAAACUCC CU UCAAGGACAUCGUCCGGG UGGAAUUA | 10531 |
| 491 | AAUUCCAG G AUCAUCAA | 2044 | UUGAUGAU GGAGGAAACUCC CU UCAAGGACAUCGUCCGGG CUGGAAUU | 10532 |
| 511 | CCAGCACC G GACCAUGC | 2045 | GGAUGGUC GGAGGAAACUCC CU UCAAGGACAUCGUCCGGG GGUGCUGG | 10533 |
| 512 | CAGCACCG G ACCAUGCA | 2046 | UGCAUGGU GGAGGAAACUCC CU UCAAGGACAUCGUCCGGG CGGUGCUG | 10534 |
| 544 | CUGCUCAA G GAACCUCU | 2047 | AGAGGUUC GGAGGAAACUCC CU UCAAGGACAUCGUCCGGG UUGAGCAG | 10535 |
| 545 | UGCUCAAG G AACCUCUA | 2048 | UAGAGGUU GGAGGAAACUCC CU UCAAGGACAUCGUCCGGG CUUGAGCA | 10536 |
| 585 | AAACCUAC G GACGGAAA | 2049 | UUUCCGUC GGAGGAAACUCC CU UCAAGGACAUCGUCCGGG GUAGGUUU | 10537 |
| 586 | AACCUACG G ACGGAAAC | 2050 | GUUUCCGU GGAGGAAACUCC CU UCAAGGACAUCGUCCGGG CGUAGGUU | 10538 |
| 589 | CUACGGAC G GAAACUGC | 2051 | GCAGUUUC GGAGGAAACUCC CU UCAAGGACAUCGUCCGGG GUCCGUAG | 10539 |
| 590 | UACGGACG G AAACUGCA | 2052 | UGCAGUUU GGAGGAAACUCC CU UCAAGGACAUCGUCCGGG CGUCCGUA | 10540 |
| 623 | AUCAUCUU G GGCUUUCG | 2053 | CGAAAGCC GGAGGAAACUCC CU UCAAGGACAUCGUCCGGG AAGAUGAU | 10541 |
| 624 | UCAUCUUG G GCUUUCGC | 2054 | GCGAAAGC GGAGGAAACUCC CU UCAAGGACAUCGUCCGGG CAAGAUGA | 10542 |
| 644 | AUACCUAU G GGAGUGGG | 2055 | CCCACUCC GGAGGAAACUCC CU UCAAGGACAUCGUCCGGG AUAGGUAU | 10543 |
| 645 | UACCUAUG G GAGUGGGC | 2056 | GCCCACUC GGAGGAAACUCC CU UCAAGGACAUCGUCCGGG CAUAGGUA | 10544 |
| 646 | ACCUAUGG G AGUGGGCC | 2057 | GGCCCACU GGAGGAAACUCC CU UCAAGGACAUCGUCCGGG CCAUAGGU | 10545 |
| 650 | AUGGGAGU G GGCCUCAG | 2058 | CUGAGGCC GGAGGAAACUCC CU UCAAGGACAUCGUCCGGG ACUCCCAU | 10546 |
| 651 | UGGGAGUG G GCCUCAGU | 2059 | ACUGAGGC GGAGGAAACUCC CU UCAAGGACAUCGUCCGGG CACUCCCA | 10547 |
| 671 | UUUCUCUU G GCUCAGUU | 2060 | AACUGAGC GGAGGAAACUCC CU UCAAGGACAUCGUCCGGG AAGAGAAA | 10548 |
| 701 | UGUUCAGU G GUUCGUAG | 2061 | CUACGAAC GGAGGAAACUCC CU UCAAGGACAUCGUCCGGG ACUGAACA | 10549 |
| 709 | GGUUCGUA G GGCUUUCC | 2062 | GGAAAGCC GGAGGAAACUCC CU UCAAGGACAUCGUCCGGG UACGAACC | 10550 |
| 710 | GUUCGUAG G GCUUUCCC | 2063 | GGGAAAGC GGAGGAAACUCC CU UCAAGGACAUCGUCCGGG CUACGAAC | 10551 |
| 728 | CACUGUCU G GCUUUCAG | 2064 | CUGAAAGC GGAGGAAACUCC CU UCAAGGACAUCGUCCGGG AGACAGUG | 10552 |
| 743 | AGUUAUAU G GAUGAUGU | 2065 | ACAUCAUC GGAGGAAACUCC CU UCAAGGACAUCGUCCGGG AUAUAACU | 10553 |
| 744 | GUUAUAUG G AUGAUGUG | 2066 | CACAUCAU GGAGGAAACUCC CU UCAAGGACAUCGUCCGGG CAUAUAAC | 10554 |
| 752 | GAUGAUGU G GUUUUGGG | 2067 | CCCAAAAC GGAGGAAACUCC CU UCAAGGACAUCGUCCGGG ACAUCAUC | 10555 |
| 758 | GUGGUUUU G GGGGCCAA | 2068 | UUGGCCCC GGAGGAAACUCC CU UCAAGGACAUCGUCCGGG AAAACCAC | 10556 |
| 759 | UGGUUUUG G GGGCCAAG | 2069 | CUUGGCCC GGAGGAAACUCC CU UCAAGGACAUCGUCCGGG CAAAACCA | 10557 |
| 760 | GUUUUUGG G GGCCAAGU | 2070 | ACUUGGCC GGAGGAAACUCC CU UCAAGGACAUCGUCCGGG CCAAAACC | 10558 |
| 761 | GUUUUGGG G GCCAAGUC | 2071 | GACUUGGC GGAGGAAACUCC CU UCAAGGACAUCGUCCGGG CCCAAAAC | 10559 |
| 824 | UUGUCUUU G GGUAUACA | 2072 | UGUAUACC GGAGGAAACUCC CU UCAAGGACAUCGUCCGGG AAAGACAA | 10560 |
| 825 | UGUCUUUG G GUAUACAU | 2073 | AUGUAUAC GGAGGAAACUCC CU UCAAGGACAUCGUCCGGG CAAAGACA | 10561 |
| 856 | AACAAAAA G AUGGGGAU | 2074 | AUCCCCAU GGAGGAAACUCC CU UCAAGGACAUCGUCCGGG UUUUUGUU | 10562 |
| 859 | AAAAAGAU G GGGAUAUU | 2075 | AAUAUCCC GGAGGAAACUCC CU UCAAGGACAUCGUCCGGG AUCUUUUU | 10563 |
| 860 | AAAAGAUG G GGAUAUUC | 2076 | GAAUAUCC GGAGGAAACUCC CU UCAAGGACAUCGUCCGGG CAUCUUUU | 10564 |
| 861 | AAAGAUGG G GAUAUUCC | 2077 | GGAAUAUC GGAGGAAACUCC CU UCAAGGACAUCGUCCGGG CCAUCUUU | 10565 |
| 862 | AAGAUGGG G AUAUUCCC | 2078 | GGGAAUAU GGAGGAAACUCC CU UCAAGGACAUCGUCCGGG CCCAUCUU | 10566 |
| 881 | AACUUCAU G GGAUAUGU | 2079 | ACAUAUCC GGAGGAAACUCC CU UCAAGGACAUCGUCCGGG AUGAAGUU | 10567 |
| 882 | ACUUCAUG G GAUAUGUA | 2080 | UACAUAUC GGAGGAAACUCC CU UCAAGGACAUCGUCCGGG CAUGAAGU | 10568 |
| 883 | CUUCAUGG G AUAUGUAA | 2081 | UUACAUAU GGAGGAAACUCC CU UCAAGGACAUCGUCCGGG CCAUGAAG | 10569 |
| 894 | AUGUAAUU G GGAGUUGG | 2082 | CCAACUCC GGAGGAAACUCC CU UCAAGGACAUCGUCCGGG AAUUACAU | 10570 |
| 895 | UGUAAUUG G GAGUUGGG | 2083 | CCCAACUC GGAGGAAACUCC CU UCAAGGACAUCGUCCGGG CAAUUACA | 10571 |
| 896 | GUAAUUGG G AGUUGGGG | 2084 | CCCCAACU GGAGGAAACUCC CU UCAAGGACAUCGUCCGGG CCAAUUAC | 10572 |
| 901 | UGGGAGUU G GGGCACAU | 2085 | AUGUGCCC GGAGGAAACUCC CU UCAAGGACAUCGUCCGGG AACUCCCA | 10573 |
| 902 | GGGAGUUG G GGCACAUU | 2086 | AAUGUGCC GGAGGAAACUCC CU UCAAGGACAUCGUCCGGG CAACUCCC | 10574 |
| 903 | GGAGUUGG G GCACAUUG | 2087 | CAAUGUGC GGAGGAAACUCC CU UCAAGGACAUCGUCCGGG CCAACUCC | 10575 |
| 917 | UUGCCACA G GAACAUAU | 2088 | AUAUGUUC GGAGGAAACUCC CU UCAAGGACAUCGUCCGGG UGUGGCAA | 10576 |
| 918 | UGCCACAG G AACAUAUU | 2089 | AAUAUGUU GGAGGAAACUCC CU UCAAGGACAUCGUCCGGG CUGUGGCA | 10577 |
| 952 | GUGUUUUA G GAAACUUC | 2090 | GAAGUUUC GGAGGAAACUCC CU UCAAGGACAUCGUCCGGG UAAAACAC | 10578 |
| 953 | UGUUUUAG G AAACUUCC | 2091 | GGAAGUUU GGAGGAAACUCC CU UCAAGGACAUCGUCCGGG CUAAAACA | 10579 |
| 970 | UGUAAACA G GCCUAUUG | 2092 | CAAUAGGC GGAGGAAACUCC CU UCAAGGACAUCGUCCGGG UGUUUACA | 10580 |
| 982 | UAUUGAUU G GAAAGUAU | 2093 | AUACUUUC GGAGGAAACUCC CU UCAAGGACAUCGUCCGGG AAUCAAUA | 10581 |
| 983 | AUUGAUUG G AAAGUAUG | 2094 | CAUACUUU GGAGGAAACUCC CU UCAAGGACAUCGUCCGGG CAAUCAAU | 10582 |
| 1004 | CGAAUUGU G GGUCUUUU | 2095 | AAAAGACC GGAGGAAACUCC CU UCAAGGACAUCGUCCGGG ACAAUUCG | 10583 |
| 1005 | GAAUUGUG G GUCUUUUG | 2096 | CAAAAGAC GGAGGAAACUCC CU UCAAGGACAUCGUCCGGG CACAAUUC | 10584 |
| 1013 | GGUCUUUU G GGGUUUGC | 2097 | GCAAACCC GGAGGAAACUCC CU UCAAGGACAUCGUCCGGG AAAAGACC | 10585 |
| 1014 | GUCUUUUG G GGUUUGCC | 2098 | GGCAAACC GGAGGAAACUCC CU UCAAGGACAUCGUCCGGG CAAAAGAC | 10586 |
| 1015 | UCUUUUGG G GUUUGCCG | 2099 | CGGCAAAC GGAGGAAACUCC CU UCAAGGACAUCGUCCGGG CCAAAAGA | 10587 |
| 1041 | CGCAAUGU G GAUAUUCU | 2100 | AGAAUAUC GGAGGAAACUCC CU UCAAGGACAUCGUCCGGG ACAUUGCG | 10588 |
| 1042 | GCAAUGUG G AUAUUCUG | 2101 | CAGAAUAU GGAGGAAACUCC CU UCAAGGACAUCGUCCGGG CACAUUGC | 10589 |
| 1088 | GCAAAACA G GCUUUUAC | 2102 | GUAAAAGC GGAGGAAACUCC CU UCAAGGACAUCGUCCGGG UGUUUUGC | 10590 |
| 1115 | ACUUACAA G GCCUUUCU | 2103 | AGAAAGGC GGAGGAAACUCC CU UCAAGGACAUCGUCCGGG UUGUAAGU | 10591 |
| 1159 | CGUUGCUC G GCAACGGC | 2104 | GCCGUUGC GGAGGAAACUCC CU UCAAGGACAUCGUCCGGG GAGCAACG | 10592 |
| 1165 | UCGGCAAC G GCCUGGUC | 2105 | GACCAGGC GGAGGAAACUCC CU UCAAGGACAUCGUCCGGG GUUGCCGA | 10593 |
| 1170 | AACGGCCU G GUCUAUGC | 2106 | GCAUAGAC GGAGGAAACUCC CU UCAAGGACAUCGUCCGGG AGGCCGUU | 10594 |
| 1206 | CCCCCACU G GUUGGGGC | 2107 | GCCCCAAC GGAGGAAACUCC CU UCAAGGACAUCGUCCGGG AGUGGGGG | 10595 |
| 1210 | CACUGGUU G GGGCUUGG | 2108 | CCAAGCCC GGAGGAAACUCC CU UCAAGGACAUCGUCCGGG AACCAGUG | 10596 |
| 1211 | ACUGGUUG G GGCUUGGC | 2109 | GCCAAGCC GGAGGAAACUCC CU UCAAGGACAUCGUCCGGG CAACCAGU | 10597 |
| 1212 | CUGGUUGG G GCUUGGCC | 2110 | GGCCAAGC GGAGGAAACUCC CU UCAAGGACAUCGUCCGGG CCAACCAG | 10598 |
| 1217 | UGGGGCUU G GCCAUAGG | 2111 | CCUAUGGC GGAGGAAACUCC CU UCAAGGACAUCGUCCGGG AAGCCCCA | 10599 |
| 1224 | UGGCCAUA G GCCAUCAG | 2112 | CUGAUGGC GGAGGAAACUCC CU UCAAGGACAUCGUCCGGG UAUGGCCA | 10600 |
| 1242 | GCAUGCGU G GAACCUUU | 2113 | AAAGGUUC GGAGGAAACUCC CU UCAAGGACAUCGUCCGGG ACGCAUGC | 10601 |
| 1243 | CAUGCGUG G AACCUUUG | 2114 | CAAAGGUU GGAGGAAACUCC CU UCAAGGACAUCGUCCGGG CACGCAUG | 10602 |
| 1277 | CAUACCGC G GAACUCCU | 2115 | AGGAGUUC GGAGGAAACUCC CU UCAAGGACAUCGUCCGGG GCGGUAUG | 10603 |
| 1278 | AUACCGCG G AACUCCUA | 2116 | UAGGAGUU GGAGGAAACUCC CU UCAAGGACAUCGUCCGGG CGCGGUAU | 10604 |
| 1309 | UCGCAGCA G GUCUGGGG | 2117 | CCCCAGAC GGAGGAAACUCC CU UCAAGGACAUCGUCCGGG UGCUGCGA | 10605 |
| 1314 | GCAGGUCU G GGGCAAAA | 2118 | UUUUGCCC GGAGGAAACUCC CU UCAAGGACAUCGUCCGGG AGACCUGC | 10606 |
| 1315 | CAGGUCUG G GGCAAAAC | 2119 | GUUUUGCC GGAGGAAACUCC CU UCAAGGACAUCGUCCGGG CAGACCUG | 10607 |
| 1316 | AGGUCUGG G GCAAAACU | 2120 | AGUUUUGC GGAGGAAACUCC CU UCAAGGACAUCGUCCGGG CCAGACCU | 10608 |
| 1329 | AACUCAUC G GGACUGAC | 2121 | GUCAGUCC GGAGGAAACUCC CU UCAAGGACAUCGUCCGGG GAUGAGUU | 10609 |
| 1330 | ACUCAUCG G GACUGACA | 2122 | UGUCAGUC GGAGGAAACUCC CU UCAAGGACAUCGUCCGGG CGAUGAGU | 10610 |
| 1331 | CUCAUCGG G ACUGACAA | 2123 | UUGUCAGU GGAGGAAACUCC CU UCAAGGACAUCGUCCGGG CCGAUGAG | 10611 |
| 1378 | AUUUCCAU G GCUGCUAG | 2124 | CUAGCAGC GGAGGAAACUCC CU UCAAGGACAUCGUCCGGG AUGGAAAU | 10612 |
| 1386 | GGCUGCUA G GCUGUGCU | 2125 | AGCACAGC GGAGGAAACUCC CU UCAAGGACAUCGUCCGGG UAGCAGCC | 10613 |
| 1402 | UGCCAACU G GAUCCUAC | 2126 | GUAGGAUC GGAGGAAACUCC CU UCAAGGACAUCGUCCGGG AGUUGGCA | 10614 |
| 1403 | GCCAACUG G AUCCUACG | 2127 | CGUAGGAU GGAGGAAACUCC CU UCAAGGACAUCGUCCGGG CAGUUGGC | 10615 |
| 1413 | UCCUACGC G GGACGUCC | 2128 | GGACGUCC GGAGGAAACUCC CU UCAAGGACAUCGUCCGGG GCGUAGGA | 10616 |
| 1414 | CCUACGCG G GACGUCCU | 2129 | AGGACGUC GGAGGAAACUCC CU UCAAGGACAUCGUCCGGG CGCGUAGG | 10617 |
| 1415 | CUACGCGG G ACGUCCUU | 2130 | AAGGACGU GGAGGAAACUCC CU UCAAGGACAUCGUCCGGG CCGCGUAG | 10618 |
| 1439 | GUCCCGUC G GCGCUGAA | 2131 | UUCAGCGC GGAGGAAACUCC CU UCAAGGACAUCGUCCGGG GACGGGAC | 10619 |
| 1454 | AAUCCCGC G GACGACCC | 2132 | GGGUCGUC GGAGGAAACUCC CU UCAAGGACAUCGUCCGGG GCGGGAUU | 10620 |
| 1455 | AUCCCGCG G ACGACCCC | 2133 | GGGGUCGU GGAGGAAACUCC CU UCAAGGACAUCGUCCGGG CGCGGGAU | 10621 |
| 1468 | CCCCUCCC G GGGCCGCU | 2134 | AGCGGCCC GGAGGAAACUCC CU UCAAGGACAUCGUCCGGG GGGAGGGG | 10622 |
| 1469 | CCCUCCCG G GGCCGCUU | 2135 | AAGCGGCC GGAGGAAACUCC CU UCAAGGACAUCGUCCGGG CGGGAGGG | 10623 |
| 1470 | CCUCCCGG G GCCGCUUG | 2136 | CAAGCGGC GGAGGAAACUCC CU UCAAGGACAUCGUCCGGG CCGGGAGG | 10624 |
| 1478 | GGCCGCUU G GGGCUCUA | 2137 | UAGAGCCC GGAGGAAACUCC CU UCAAGGACAUCGUCCGGG AAGCGGCC | 10625 |
| 1479 | GCCGCUUG G GGCUCUAC | 2138 | GUAGAGCC GGAGGAAACUCC CU UCAAGGACAUCGUCCGGG CAAGCGGC | 10626 |
| 1480 | CCGCUUGG G GCUCUACC | 2139 | GGUAGAGC GGAGGAAACUCC CU UCAAGGACAUCGUCCGGG CCAAGCGG | 10627 |
| 1523 | CCGUCCAC G GGGCGCAC | 2140 | GUGCGCCC GGAGGAAACUCC CU UCAAGGACAUCGUCCGGG GUGGACGG | 10628 |
| 1524 | CGUCCACG G GGCGCACC | 2141 | GGUGCGCC GGAGGAAACUCC CU UCAAGGACAUCGUCCGGG CGUGGACG | 10629 |
| 1525 | GUCCACGG G GCGCACCU | 2142 | AGGUGCGC GGAGGAAACUCC CU UCAAGGACAUCGUCCGGG CCGUGGAC | 10630 |
| 1544 | CUUUACGC G GACUCCCC | 2143 | GGGGAGUC GGAGGAAACUCC CU UCAAGGACAUCGUCCGGG GCGUAAAG | 10631 |
| 1545 | UUUACGCG G ACUCCCCG | 2144 | CGGGGAGU GGAGGAAACUCC CU UCAAGGACAUCGUCCGGG CGCGUAAA | 10632 |
| 1574 | CAUCUGCC G GACCGUGU | 2145 | ACACGGUC GGAGGAAACUCC CU UCAAGGACAUCGUCCGGG GGCAGAUG | 10633 |
| 1575 | AUCUGCCG G ACCGUGUG | 2146 | CACACGGU GGAGGAAACUCC CU UCAAGGACAUCGUCCGGG CGGCAGAU | 10634 |
| 1612 | CGUCGCAU G GAGACCAC | 2147 | GUGGUCUC GGAGGAAACUCC CU UCAAGGACAUCGUCCGGG AUGCGACG | 10635 |
| 1613 | GUCGCAUG G AGACCACC | 2148 | GGUGGUCU GGAGGAAACUCC CU UCAAGGACAUCGUCCGGG CAUGCGAC | 10636 |
| 1615 | CGCAUGGA G ACCACCGU | 2149 | ACGGUGGU GGAGGAAACUCC CU UCAAGGACAUCGUCCGGG UCCAUGCG | 10637 |
| 1635 | CGCCCACA G GAACCUGC | 2150 | GCAGGUUC GGAGGAAACUCC CU UCAAGGACAUCGUCCGGG UGUGGGCG | 10638 |
| 1636 | GCCCACAG G AACCUGCC | 2151 | GGCAGGUU GGAGGAAACUCC CU UCAAGGACAUCGUCCGGG CUGUGGGC | 10639 |
| 1648 | CUGCCCAA G GUCUUGCA | 2152 | UGCAAGAC GGAGGAAACUCC CU UCAAGGACAUCGUCCGGG UUGGGCAG | 10640 |
| 1660 | UUGCAUAA G AGGACUCU | 2153 | AGAGUCCU GGAGGAAACUCC CU UCAAGGACAUCGUCCGGG UUAUGCAA | 10641 |
| 1662 | GCAUAAGA G GACUCUUG | 2154 | CAAGAGUC GGAGGAAACUCC CU UCAAGGACAUCGUCCGGG UCUUAUGC | 10642 |
| 1663 | CAUAAGAG G ACUCUUGG | 2155 | CCAAGAGU GGAGGAAACUCC CU UCAAGGACAUCGUCCGGG CUCUUAUG | 10643 |
| 1670 | GGACUCUU G GACUUUCA | 2156 | UGAAAGUC GGAGGAAACUCC CU UCAAGGACAUCGUCCGGG AAGAGUCC | 10644 |
| 1671 | GACUCUUG G ACUUUCAG | 2157 | CUGAAAGU GGAGGAAACUCC CU UCAAGGACAUCGUCCGGG CAAGAGUC | 10645 |
| 1702 | GACCUUGA G GCAUACUU | 2158 | AAGUAUGC GGAGGAAACUCC CU UCAAGGACAUCGUCCGGG UCAAGGUC | 10646 |
| 1715 | ACUUCAAA G ACUGUGUG | 2159 | CACACAGU GGAGGAAACUCC CU UCAAGGACAUCGUCCGGG UUUGAAGU | 10647 |
| 1734 | UAAUGAGU G GGAGGAGU | 2160 | ACUCCUCC GGAGGAAACUCC CU UCAAGGACAUCGUCCGGG ACUCAUUA | 10648 |
| 1735 | AAUGAGUG G GAGGAGUU | 2161 | AACUCCUC GGAGGAAACUCC CU UCAAGGACAUCGUCCGGG CACUCAUU | 10649 |
| 1736 | AUGAGUGG G AGGAGUUG | 2162 | CAACUCCU GGAGGAAACUCC CU UCAAGGACAUCGUCCGGG CCACUCAU | 10650 |
| 1738 | GAGUGGGA G GAGUUGGG | 2163 | CCCAACUC GGAGGAAACUCC CU UCAAGGACAUCGUCCGGG UCCCACUC | 10651 |
| 1739 | AGUGGGAG G AGUUGGGG | 2164 | CCCCAACU GGAGGAAACUCC CU UCAAGGACAUCGUCCGGG CUCCCACU | 10652 |
| 1744 | GAGGAGUU G GGGGAGGA | 2165 | UCCUCCCC GGAGGAAACUCC CU UCAAGGACAUCGUCCGGG AACUCCUC | 10653 |
| 1745 | AGGAGUUG G GGGAGGAG | 2166 | CUCCUCCC GGAGGAAACUCC CU UCAAGGACAUCGUCCGGG CAACUCCU | 10654 |
| 1746 | GGAGUUGG G GGAGGAGG | 2167 | CCUCCUCC GGAGGAAACUCC CU UCAAGGACAUCGUCCGGG CCAACUCC | 10655 |
| 1747 | GAGUUGGG G GAGGAGGU | 2168 | ACCUCCUC GGAGGAAACUCC CU UCAAGGACAUCGUCCGGG CCCAACUC | 10656 |
| 1748 | AGUUGGGG G AGGAGGUU | 2169 | AACCUCCU GGAGGAAACUCC CU UCAAGGACAUCGUCCGGG CCCCAACU | 10657 |
| 1750 | UUGGGGGA G GAGGUUAG | 2170 | CUAACCUC GGAGGAAACUCC CU UCAAGGACAUCGUCCGGG UCCCCCAA | 10658 |
| 1751 | UGGGGGAG G AGGUUAGG | 2171 | CCUAACCU GGAGGAAACUCC CU UCAAGGACAUCGUCCGGG CUCCCCCA | 10659 |
| 1753 | GGGGAGGA G GUUAGGUU | 2172 | AACCUAAC GGAGGAAACUCC CU UCAAGGACAUCGUCCGGG UCCUCCCC | 10660 |
| 1758 | GGAGGUUA G GUUAAAGG | 2173 | CCUUUAAC GGAGGAAACUCC CU UCAAGGACAUCGUCCGGG UAACCUCC | 10661 |
| 1765 | AGGUUAAA G GUCUUUGU | 2174 | ACAAAGAC GGAGGAAACUCC CU UCAAGGACAUCGUCCGGG UUUAACCU | 10662 |
| 1778 | UUGUACUA G GAGGCUGU | 2175 | ACAGCCUC GGAGGAAACUCC CU UCAAGGACAUCGUCCGGG UAGUACAA | 10663 |
| 1779 | UGUACUAG G AGGCUGUA | 2176 | UACAGCCU GGAGGAAACUCC CU UCAAGGACAUCGUCCGGG CUAGUACA | 10664 |
| 1781 | UACUAGGA G GCUGUAGG | 2177 | CCUACAGC GGAGGAAACUCC CU UCAAGGACAUCGUCCGGG UCCUAGUA | 10665 |
| 1788 | AGGCUGUA G GCAUAAAU | 2178 | AUUUAUGC GGAGGAAACUCC CU UCAAGGACAUCGUCCGGG UACAGCCU | 10666 |
| 1798 | CAUAAAUU G GUGUGUUC | 2179 | GAACACAC GGAGGAAACUCC CU UCAAGGACAUCGUCCGGG AAUUUAUG | 10667 |
| 1888 | UGUGCCUU G GGUGGCUU | 2180 | AAGCCACC GGAGGAAACUCC CU UCAAGGACAUCGUCCGGG AAGGCACA | 10668 |
| 1889 | GUGCCUUG G GUGGCUUU | 2181 | AAAGCCAC GGAGGAAACUCC CU UCAAGGACAUCGUCCGGG CAAGGCAC | 10669 |
| 1892 | CCUUGGGU G GCUUUGGG | 2182 | CCCAAAGC GGAGGAAACUCC CU UCAAGGACAUCGUCCGGG ACCCAAGG | 10670 |
| 1898 | GUGGCUUU G GGGCAUGG | 2183 | CCAUGCCC GGAGGAAACUCC CU UCAAGGACAUCGUCCGGG AAAGCCAC | 10671 |
| 1899 | UGGCUUUG G GGCAUGGA | 2184 | UCCAUGCC GGAGGAAACUCC CU UCAAGGACAUCGUCCGGG CAAAGCCA | 10672 |
| 1900 | GGCUUUGG G GCAUGGAC | 2185 | GUCCAUGC GGAGGAAACUCC CU UCAAGGACAUCGUCCGGG CCAAAGCC | 10673 |
| 1905 | UGGGGCAU G GACAUUGA | 2186 | UCAAUGUC GGAGGAAACUCC CU UCAAGGACAUCGUCCGGG AUGCCCCA | 10674 |
| 1906 | GGGGCAUG G ACAUUGAC | 2187 | GUCAAUGU GGAGGAAACUCC CU UCAAGGACAUCGUCCGGG CAUGCCCC | 10675 |
| 1924 | CGUAUAAA G AAUUUGGA | 2188 | UCCAAAUU GGAGGAAACUCC CU UCAAGGACAUCGUCCGGG UUUAUACG | 10676 |
| 1930 | AAGAAUUU G GAGCUUCU | 2189 | AGAAGCUC GGAGGAAACUCC CU UCAAGGACAUCGUCCGGG AAAUUCUU | 10677 |
| 1931 | AGAAUUUG G AGCUUCUG | 2190 | CAGAAGCU GGAGGAAACUCC CU UCAAGGACAUCGUCCGGG CAAAUUCU | 10678 |
| 1941 | GCUUCUGU G GAGUUACU | 2191 | AGUAACUC GGAGGAAACUCC CU UCAAGGACAUCGUCCGGG ACAGAAGC | 10679 |
| 1942 | CUUCUGUG G AGUUACUC | 2192 | GAGUAACU GGAGGAAACUCC CU UCAAGGACAUCGUCCGGG CACAGAAG | 10680 |
| 1987 | CUAUUCGA G AUCUCCUC | 2193 | GAGGAGAU GGAGGAAACUCC CU UCAAGGACAUCGUCCGGG UCGAAUAG | 10681 |
| 2018 | UCUGUAUC G GGGGGCCU | 2194 | AGGCCCCC GGAGGAAACUCC CU UCAAGGACAUCGUCCGGG GAUACAGA | 10682 |
| 2019 | CUGUAUCG G GGGCCCUU | 2195 | AAGGCCCC GGAGGAAACUCC CU UCAAGGACAUCGUCCGGG CGAUACAG | 10683 |
| 2020 | UGUAUCGG G GGGCCUUA | 2196 | UAAGGCCC GGAGGAAACUCC CU UCAAGGACAUCGUCCGGG CCGAUACA | 10684 |
| 2021 | GUAUCGGG G GGCCUUAG | 2197 | CUAAGGCC GGAGGAAACUCC CU UCAAGGACAUCGUCCGGG CCCGAUAC | 10685 |
| 2022 | UAUCGGGG G GCCUUAGA | 2198 | UCUAAGGC GGAGGAAACUCC CU UCAAGGACAUCGUCCGGG CCCCGAUA | 10686 |
| 2029 | GGGCCUUA G AGUCUCCC | 2199 | CGGAGACU GGAGGAAACUCC CU UCAAGGACAUCGUCCGGG UAAGGCCC | 10687 |
| 2037 | GAGUCUCC G GAACAUUG | 2200 | CAAUGUUC GGAGGAAACUCC CU UCAAGGACAUCGUCCGGG GGAGACUC | 10688 |
| 2038 | AGUCUCCG G AACAUUGU | 2201 | ACAAUGUU GGAGGAAACUCC CU UCAAGGACAUCGUCCGGG CGGAGACU | 10689 |
| 2061 | CACCAUAC G GCACUCAG | 2202 | CUGAGUGC GGAGGAAACUCC CU UCAAGGACAUCGUCCGGG GUAUGGUG | 10690 |
| 2069 | GGCACUCA G GCAAGCUA | 2203 | UAGCUUGC GGAGGAAACUCC CU UCAAGGACAUCGUCCGGG UGAGUGCC | 10691 |
| 2087 | UCUGUGUU G GGGUGAGU | 2204 | ACUCACCC GGAGGAAACUCC CU UCAAGGACAUCGUCCGGG AACACAGA | 10692 |
| 2088 | CUGUGUUG G GGUGAGUU | 2205 | AACUCACC GGAGGAAACUCC CU UCAAGGACAUCGUCCGGG CAACACAG | 10693 |
| 2089 | UGUGUUGG G GUGAGUUG | 2206 | CAACUCAC GGAGGAAACUCC CU UCAAGGACAUCGUCCGGG CCAACACA | 10694 |
| 2114 | AGCCACCU G GGUGGGAA | 2207 | UUCCCACC GGAGGAAACUCC CU UCAAGGACAUCGUCCGGG AGGUGGCU | 10695 |
| 2115 | GCCACCUG G GUGGGAAG | 2208 | CUUCCCAC GGAGGAAACUCC CU UCAAGGACAUCGUCCGGG CAGGUGGC | 10696 |
| 2118 | ACCUGGGU G GGAAGUAA | 2209 | UUACUUCC GGAGGAAACUCC CU UCAAGGACAUCGUCCGGG ACCCAGGU | 10697 |
| 2119 | CCUGGGUG G GAAGUAAU | 2210 | AUUACUUC GGAGGAAACUCC CU UCAAGGACAUCGUCCGGG CACCCAGG | 10698 |
| 2120 | CUGGGUGG G AAGUAAUU | 2211 | AAUUACUU GGAGGAAACUCC CU UCAAGGACAUCGUCCGGG CCACCCAG | 10699 |
| 2130 | AGUAAUUU G GAAGAUCC | 2212 | GGAUCUUC GGAGGAAACUCC CU UCAAGGACAUCGUCCGGG AAAUUACU | 10700 |
| 2131 | GUAAUUUG G AAGAUCCA | 2213 | UGGAUCUU GGAGGAAACUCC CU UCAAGGACAUCGUCCGGG CAAAUUAC | 10701 |
| 2134 | AUUUGGAA G AUCCAGCA | 2214 | UGCUGGAU GGAGGAAACUCC CU UCAAGGACAUCGUCCGGG UUCCAAAU | 10702 |
| 2147 | AGCAUCCA G GGAAUUAG | 2215 | CUAAUUCC GGAGGAAACUCC CU UCAAGGACAUCGUCCGGG UGGAUGCU | 10703 |
| 2148 | GCAUCCAG G GAAUUAGU | 2216 | ACUAAUUC GGAGGAAACUCC CU UCAAGGACAUCGUCCGGG CUGGAUGC | 10704 |
| 2149 | CAUCCAGG G AAUUAGUA | 2217 | UACUAAUU GGAGGAAACUCC CU UCAAGGACAUCGUCCGGG CCUGGAUG | 10705 |
| 2181 | GUUAAUAU G GGCCUAAA | 2218 | UUUAGGCC GGAGGAAACUCC CU UCAAGGACAUCGUCCGGG AUAUUAAC | 10706 |
| 2182 | UUAAUAUG G GCCUAAAA | 2219 | UUUUAGGC GGAGGAAACUCC CU UCAAGGACAUCGUCCGGG CAUAUUAA | 10707 |
| 2195 | AAAAAUCA G ACAACUAU | 2220 | AUAGUUGU GGAGGAAACUCC CU UCAAGGACAUCGUCCGGG UGAUUUUU | 10708 |
| 2207 | ACUAUUGU G GUUUCACA | 2221 | UGUGAAAC GGAGGAAACUCC CU UCAAGGACAUCGUCCGGG ACAAUAGU | 10709 |
| 2233 | UUACUUUU G GGCGAGAA | 2222 | UUCUCGCC GGAGGAAACUCC CU UCAAGGACAUCGUCCGGG AAAAGUAA | 10710 |
| 2234 | UACUUUUG G GCGAGAAA | 2223 | UUUCUCGC GGAGGAAACUCC CU UCAAGGACAUCGUCCGGG CAAAAGUA | 10711 |
| 2239 | UUGGGCGA G AAACUGUU | 2224 | AACAGUUU GGAGGAAACUCC CU UCAAGGACAUCGUCCGGG UCGCCCAA | 10712 |
| 2259 | GAAUAUUU G GUGUCUUU | 2225 | AAAGACAC GGAGGAAACUCC CU UCAAGGACAUCGUCCGGG AAAUAUUC | 10713 |
| 2269 | UGUCUUUU G GAGUGUGG | 2226 | CCACACUC GGAGGAAACUCC CU UCAAGGACAUCGUCCGGG AAAAGACA | 10714 |
| 2270 | GUCUUUUG G AGUGUGGA | 2227 | UCCACACU GGAGGAAACUCC CU UCAAGGACAUCGUCCGGG CAAAAGAC | 10715 |
| 2276 | UGGAGUGU G GAUUCGCA | 2228 | UGCGAAUC GGAGGAAACUCC CU UCAAGGACAUCGUCCGGG ACACUCCA | 10716 |
| 2277 | GGAGUGUG G AUUCGCAC | 2229 | GUGCGAAU GGAGGAAACUCC CU UCAAGGACAUCGUCCGGG CACACUCC | 10717 |
| 2300 | UGCAUAUA G ACCACCAA | 2230 | UUGGUGGU GGAGGAAACUCC CU UCAAGGACAUCGUCCGGG UAUAUGCA | 10718 |
| 2334 | ACACUUCC G GAAACUAC | 2231 | GUAGUUUC GGAGGAAACUCC CU UCAAGGACAUCGUCCGGG GGAAGUGU | 10719 |
| 2335 | CACUUCCG G AAACUACU | 2232 | AGUAGUUU GGAGGAAACUCC CU UCAAGGACAUCGUCCGGG CGGAAGUG | 10720 |
| 2351 | UGUUGUUA G ACGAAGAG | 2233 | CUCUUCGU GGAGGAAACUCC CU UCAAGGACAUCGUCCGGG UAACAACA | 10721 |
| 2357 | UAGACGAA G AGGCAGGU | 2234 | ACCUGCCU GGAGGAAACUCC CU UCAAGGACAUCGUCCGGG UUCGUCUA | 10722 |
| 2359 | GACGAAGA G GCAGGUCC | 2235 | GGACCUGC GGAGGAAACUCC CU UCAAGGACAUCGUCCGGG UCUUCGUC | 10723 |
| 2363 | AAGAGGCA G GUCCCCUA | 2236 | UAGGGGAC GGAGGAAACUCC CU UCAAGGACAUCGUCCGGG UGCCUCUU | 10724 |
| 2372 | GUCCCCUA G AAGAAGAA | 2237 | UUCUUCUU GGAGGAAACUCC CU UCAAGGACAUCGUCCGGG UAGGGGAC | 10725 |
| 2375 | CCCUAGAA G AAGAACUC | 2238 | GAGUUCUU GGAGGAAACUCC CU UCAAGGACAUCGUCCGGG UUCUAGGG | 10726 |
| 2378 | UAGAAGAA G AACUCCCU | 2239 | AGGGAGUU GGAGGAAACUCC CU UCAAGGACAUCGUCCGGG UUCUUCUA | 10727 |
| 2396 | GCCUCGCA G ACGAAGGU | 2240 | ACCUUCGU GGAGGAAACUCC CU UCAAGGACAUCGUCCGGG UGCGAGGC | 10728 |
| 2402 | CAGACGAA G GUCUCAAU | 2241 | AUUGAGAC GGAGGAAACUCC CU UCAAGGACAUCGUCCGGG UUCGUCUG | 10729 |
| 2423 | GCGUCGCA G AAGAUCUC | 2242 | GAGAUCUU GGAGGAAACUCC CU UCAAGGACAUCGUCCGGG UGCGACGC | 10730 |
| 2426 | UCGCAGAA G AUCUCAAU | 2243 | AUUGAGAU GGAGGAAACUCC CU UCAAGGACAUCGUCCGGG UUCUGCGA | 10731 |
| 2438 | UCAAUCUC G GGAAUCUC | 2244 | GAGAUUCC GGAGGAAACUCC CU UCAAGGACAUCGUCCGGG GAGAUUGA | 10732 |
| 2439 | CAAUCUCG G GAAUCUCA | 2245 | UGAGAUUC GGAGGAAACUCC CU UCAAGGACAUCGUCCGGG CGAGAUUG | 10733 |
| 2440 | AAUCUCGG G AAUCUCAA | 2246 | UUGAGAUU GGAGGAAACUCC CU UCAAGGACAUCGUCCGGG CCGAGAUU | 10734 |
| 2463 | UAUUCCUU G GACACAUA | 2247 | UAUGUGUC GGAGGAAACUCC CU UCAAGGACAUCGUCCGGG AAGGAAUA | 10735 |
| 2464 | AUUCCUUG G ACACAUAA | 2248 | UUAUGUGU GGAGGAAACUCC CU UCAAGGACAUCGUCCGGG CAAGGAAU | 10736 |
| 2473 | ACACAUAA G GUGGGAAA | 2249 | UUUCCCAC GGAGGAAACUCC CU UCAAGGACAUCGUCCGGG UUAUGUGU | 10737 |
| 2476 | CAUAAGGU G GGAAACUU | 2250 | AAGUUUCC GGAGGAAACUCC CU UCAAGGACAUCGUCCGGG ACCUUAUG | 10738 |
| 2477 | AUAAGGUG G GAAACUUU | 2251 | AAAGUUUC GGAGGAAACUCC CU UCAAGGACAUCGUCCGGG CACCUUAU | 10739 |
| 2478 | UAAGGUGG G AAACUUUA | 2252 | UAAAGUUU GGAGGAAACUCC CU UCAAGGACAUCGUCCGGG CCACCUUA | 10740 |
| 2488 | AACUUUAC G GGGCUUUA | 2253 | UAAAGCCC GGAGGAAACUCC CU UCAAGGACAUCGUCCGGG GUAAAGUU | 10741 |
| 2489 | ACUUUACG G GGCUUUAU | 2254 | AUAAAGCC GGAGGAAACUCC CU UCAAGGACAUCGUCCGGG CGUAAAGU | 10742 |
| 2490 | CUUUACGG G GCUUUAUU | 2255 | AAUAAAGC GGAGGAAACUCC CU UCAAGGACAUCGUCCGGG CCGUAAAG | 10743 |
| 2506 | UCUUCUAC G GUACCUUG | 2256 | CAAGGUAC GGAGGAAACUCC CU UCAAGGACAUCGUCCGGG GUAGAAGA | 10744 |
| 2529 | UCCUAAAU G GCAAACUC | 2257 | GAGUUUGC GGAGGAAACUCC CU UCAAGGACAUCGUCCGGG AUUUAGGA | 10745 |
| 2563 | CAUUUGCA G GAGGACAU | 2258 | AUGUCCUC GGAGGAAACUCC CU UCAAGGACAUCGUCCGGG UGCAAAUG | 10746 |
| 2564 | AUUUGCAG G AGGACAUU | 2259 | AAUGUCCU GGAGGAAACUCC CU UCAAGGACAUCGUCCGGG CUGCAAAU | 10747 |
| 2566 | UUGCAGGA G GACAUUGU | 2260 | ACAAUGUC GGAGGAAACUCC CU UCAAGGACAUCGUCCGGG UCCUGCAA | 10748 |
| 2567 | UGCAGGAG G ACAUUGUU | 2261 | AACAAUGU GGAGGAAACUCC CU UCAAGGACAUCGUCCGGG CUCCUGCA | 10749 |
| 2580 | UGUUGAUA G AUGUAAGC | 2262 | GCUUACAU GGAGGAAACUCC CU UCAAGGACAUCGUCCGGG UAUCAACA | 10750 |
| 2596 | CAAUUUGU G GGGCCCCU | 2263 | AGGGGCCC GGAGGAAACUCC CU UCAAGGACAUCGUCCGGG ACAAAUUG | 10751 |
| 2597 | AAUUUGUG G GGCCCCUU | 2264 | AAGGGGCC GGAGGAAACUCC CU UCAAGGACAUCGUCCGGG CACAAAUU | 10752 |
| 2598 | AUUUGUGG G GCCCCUUA | 2265 | UAAGGGGC GGAGGAAACUCC CU UCAAGGACAUCGUCCGGG CCACAAAU | 10753 |
| 2622 | UGAAAACA G GAGACUUA | 2266 | UAAGUCUC GGAGGAAACUCC CU UCAAGGACAUCGUCCGGG UGUUUUCA | 10754 |
| 2623 | GAAAACAG G AGACUUAA | 2267 | UUAAGUCU GGAGGAAACUCC CU UCAAGGACAUCGUCCGGG CUGUUUUC | 10755 |
| 2625 | AAACAGGA G ACUUAAAU | 2268 | AUUUAAGU GGAGGAAACUCC CU UCAAGGACAUCGUCCGGG UCCUGUUU | 10756 |
| 2649 | GCCUGCUA G GUUUUAUC | 2269 | GAUAAAAC GGAGGAAACUCC CU UCAAGGACAUCGUCCGGG UAGCAGGC | 10757 |
| 2684 | UGCCCUUA G AUAAAGGG | 2270 | CCCUUUAU GGAGGAAACUCC CU UCAAGGACAUCGUCCGGG UAAGGGCA | 10758 |
| 2690 | UAGAUAAA G GGAUCAAA | 2271 | UUUGAUCC GGAGGAAACUCC CU UCAAGGACAUCGUCCGGG UUUAUCUA | 10759 |
| 2691 | AGAUAAAG G GAUCAAAC | 2272 | GUUUGAUC GGAGGAAACUCC CU UCAAGGACAUCGUCCGGG CUUUAUCU | 10760 |
| 2692 | GAUAAAGG G AUCAAACC | 2273 | GGUUUGAU GGAGGAAACUCC CU UCAAGGACAUCGUCCGGG CCUUUAUC | 10761 |
| 2711 | AUUAUCCA G AGUAUGUA | 2274 | UACAUACU GGAGGAAACUCC CU UCAAGGACAUCGUCCGGG UGGAUAAU | 10762 |
| 2737 | UACUUCCA G ACGCGACA | 2275 | UGUCGCGU GGAGGAAACUCC CU UCAAGGACAUCGUCCGGG UGGAAGUA | 10763 |
| 2763 | CACUCUUU G GAAGGCGG | 2276 | CCGCCUUC GGAGGAAACUCC CU UCAAGGACAUCGUCCGGG AAAGAGUG | 10764 |
| 2764 | ACUCUUUG G AAGGCGGG | 2277 | CCCGCCUU GGAGGAAACUCC CU UCAAGGACAUCGUCCGGG CAAAGAGU | 10765 |
| 2767 | CUUUGGAA G GCGGGGAU | 2278 | AUCCCCGC GGAGGAAACUCC CU UCAAGGACAUCGUCCGGG UUCCAAAG | 10766 |
| 2770 | UGGAAGGC G GGGAUCUU | 2279 | AAGAUCCC GGAGGAAACUCC CU UCAAGGACAUCGUCCGGG GCCUUCCA | 10767 |
| 2771 | GGAAGGCG G GGAUCUUA | 2280 | UAAGAUCC GGAGGAAACUCC CU UCAAGGACAUCGUCCGGG CGCCUUCC | 10768 |
| 2772 | GAAGGCGG G GAUCUUAU | 2281 | AUAAGAUC GGAGGAAACUCC CU UCAAGGACAUCGUCCGGG CCGCCUUC | 10769 |
| 2773 | AAGGCGGG G AUCUUAUA | 2282 | UAUAAGAU GGAGGAAACUCC CU UCAAGGACAUCGUCCGGG CCCGCCUU | 10770 |
| 2787 | AUAUAAAA G AGAGUCCA | 2283 | UGGACUCU GGAGGAAACUCC CU UCAAGGACAUCGUCCGGG UUUUAUAU | 10771 |
| 2789 | AUAAAAGA G AGUCCACA | 2284 | UGUGGACU GGAGGAAACUCC CU UCAAGGACAUCGUCCGGG UCUUUUAU | 10772 |
| 2816 | CAUUUUGC G GGUCACCA | 2285 | UGGUGACC GGAGGAAACUCC CU UCAAGGACAUCGUCCGGG GCAAAAUG | 10773 |
| 2817 | AUUUUGCG G GUCACCAU | 2286 | AUGGUGAC GGAGGAAACUCC CU UCAAGGACAUCGUCCGGG CGCAAAAU | 10774 |
| 2832 | AUAUUCUU G GGAACAAG | 2287 | CUUGUUCC GGAGGAAACUCC CU UCAAGGACAUCGUCCGGG AAGAAUAU | 10775 |
| 2833 | UAUUCUUG G GAACAAGA | 2288 | UCUUGUUC GGAGGAAACUCC CU UCAAGGACAUCGUCCGGG CAAGAAUA | 10776 |
| 2834 | AUUCUUGG G AACAAGAU | 2289 | AUCUUGUU GGAGGAAACUCC CU UCAAGGACAUCGUCCGGG CCAAGAAU | 10777 |
| 2840 | GGGAACAA G AUCUACAG | 2290 | CUGUAGAU GGAGGAAACUCC CU UCAAGGACAUCGUCCGGG UUGUUCCC | 10778 |
| 2852 | UACAGCAU G GGAGGUUG | 2291 | CAACCUCC GGAGGAAACUCC CU UCAAGGACAUCGUCCGGG AUGCUGUA | 10779 |
| 2853 | ACAGCAUG G GAGGUUGG | 2292 | CCAACCUC GGAGGAAACUCC CU UCAAGGACAUCGUCCGGG CAUGCUGU | 10780 |
| 2854 | CAGCAUGG G AGGUUGGU | 2293 | ACCAACCU GGAGGAAACUCC CU UCAAGGACAUCGUCCGGG CCAUGCUG | 10781 |
| 2856 | GCAUGGGA G GUUGGUCU | 2294 | AGACCAAC GGAGGAAACUCC CU UCAAGGACAUCGUCCGGG UCCCAUGC | 10782 |
| 2860 | GGGAGGUU G GUCUUCCA | 2295 | UGGAAGAC GGAGGAAACUCC CU UCAAGGACAUCGUCCGGG AACCUCCC | 10783 |
| 2880 | CUCGAAAA G GCAUGGGG | 2296 | CCCCAUGC GGAGGAAACUCC CU UCAAGGACAUCGUCCGGG UUUUCGAG | 10784 |
| 2885 | AAAGGCAU G GGGACAAA | 2297 | UUUGUCCC GGAGGAAACUCC CU UCAAGGACAUCGUCCGGG AUGCCUUU | 10785 |
| 2886 | AAGGCAUG G GGACAAAU | 2298 | AUUUGUCC GGAGGAAACUCC CU UCAAGGACAUCGUCCGGG CAUGCCUU | 10786 |
| 2887 | AGGCAUGG G GACAAAUC | 2299 | GAUUUGUC GGAGGAAACUCC CU UCAAGGACAUCGUCCGGG CCAUGCCU | 10787 |
| 2888 | GGCAUGGG G ACAAAUCU | 2300 | AGAUUUGU GGAGGAAACUCC CU UCAAGGACAUCGUCCGGG CCCAUGCC | 10788 |
| 2915 | AAUCCCCU G GGAUUCUU | 2301 | AAGAAUCC GGAGGAAACUCC CU UCAAGGACAUCGUCCGGG AGGGGAUU | 10789 |
| 2916 | AUCCCCUG G GAUUCUUC | 2302 | GAAGAAUC GGAGGAAACUCC CU UCAAGGACAUCGUCCGGG CAGGGGAU | 10790 |
| 2917 | UCCCCUGG G AUUCUUCC | 2303 | GGAAGAAU GGAGGAAACUCC CU UCAAGGACAUCGUCCGGG CCAGGGGA | 10791 |
| 2939 | CAUCAGUU G GACCCUGC | 2304 | GCAGGGUC GGAGGAAACUCC CU UCAAGGACAUCGUCCGGG AACUGAUG | 10792 |
| 2940 | AUCAGUUG G ACCCUGCA | 2305 | UGCAGGGU GGAGGAAACUCC CU UCAAGGACAUCGUCCGGG CAACUGAU | 10793 |
| 2973 | UAAAUCCA G AUUGGGAC | 2306 | GUCCCAAU GGAGGAAACUCC CU UCAAGGACAUCGUCCGGG UGGAUUUA | 10794 |
| 2977 | UCCAGAUU G GGACCUCA | 2307 | UGAGGUCC GGAGGAAACUCC CU UCAAGGACAUCGUCCGGG AAUCUGGA | 10795 |
| 2978 | CCAGAUUG G GACCUCAA | 2308 | UUGAGGUC GGAGGAAACUCC CU UCAAGGACAUCGUCCGGG CAAUCUGG | 10796 |
| 2979 | CAGAUUGG G ACCUCAAC | 2309 | GUUGAGGU GGAGGAAACUCC CU UCAAGGACAUCGUCCGGG CCAAUCUG | 10797 |
| 2996 | CCGCACAA G GACAACUG | 2310 | CAGUUGUC GGAGGAAACUCC CU UCAAGGACAUCGUCCGGG UUGUGCGG | 10798 |
| 2997 | CGCACAAG G ACAACUGG | 2311 | CCAGUUGU GGAGGAAACUCC CU UCAAGGACAUCGUCCGGG CUUGUGCG | 10799 |
| 3004 | GGACAACU G GCCGGACG | 2312 | CGUCCGGC GGAGGAAACUCC CU UCAAGGACAUCGUCCGGG AGUUGUCC | 10800 |
| 3008 | AACUGGCC G GACGCCAA | 2313 | UUGGCGUC GGAGGAAACUCC CU UCAAGGACAUCGUCCGGG GGCCAGUU | 10801 |
| 3009 | ACUGGCCG G ACGCCAAC | 2314 | GUUGGCGU GGAGGAAACUCC CU UCAAGGACAUCGUCCGGG CGGCCAGU | 10802 |
| 3020 | GCCAACAA G GUGGGAGU | 2315 | ACUCCCAC GGAGGAAACUCC CU UCAAGGACAUCGUCCGGG UUGUUGGC | 10803 |
| 3023 | AACAAGGU G GGAGUGGG | 2316 | CCCACUCC GGAGGAAACUCC CU UCAAGGACAUCGUCCGGG ACCUUGUU | 10804 |
| 3024 | ACAAGGUG G GAGUGGGA | 2317 | UCCCACUC GGAGGAAACUCC CU UCAAGGACAUCGUCCGGG CACCUUGU | 10805 |
| 3025 | CAAGGUGG G AGUGGGAG | 2318 | CUCCCACU GGAGGAAACUCC CU UCAAGGACAUCGUCCGGG CCACCUUG | 10806 |
| 3029 | GUGGGAGU G GGAGCAUU | 2319 | AAUGCUCC GGAGGAAACUCC CU UCAAGGACAUCGUCCGGG ACUCCCAC | 10807 |
| 3030 | UGGGAGUG G GAGCAUUC | 2320 | GAAUGCUC GGAGGAAACUCC CU UCAAGGACAUCGUCCGGG CACUCCCA | 10808 |
| 3031 | GGGAGUGG G AGCAUUCG | 2321 | CGAAUGCU GGAGGAAACUCC CU UCAAGGACAUCGUCCGGG CCACUCCC | 10809 |
| 3039 | GAGCAUUC G GGCCAGGG | 2322 | CCCUGGCC GGAGGAAACUCC CU UCAAGGACAUCGUCCGGG GAAUGCUC | 10810 |
| 3040 | AGCAUUCG G GCCAGGGU | 2323 | ACCCUGGC GGAGGAAACUCC CU UCAAGGACAUCGUCCGGG CGAAUGCU | 10811 |
| 3045 | UCGGGCCA G GGUUCACC | 2324 | GGUGAACC GGAGGAAACUCC CU UCAAGGACAUCGUCCGGG UGGCCCGA | 10812 |
| 3046 | CGGGCCAG G GUUCACCC | 2325 | GGGUGAAC GGAGGAAACUCC CU UCAAGGACAUCGUCCGGG CUGGCCCG | 10813 |
| 3063 | CUCCCCAU G GGGGACUG | 2326 | CAGUCCCC GGAGGAAACUCC CU UCAAGGACAUCGUCCGGG AUGGGGAG | 10814 |
| 3064 | UCCCCAUG G GGGACUGU | 2327 | ACAGUCCC GGAGGAAACUCC CU UCAAGGACAUCGUCCGGG CAUGGGGA | 10815 |
| 3065 | CCCCAUGG G GGACUGUU | 2328 | AACAGUCC GGAGGAAACUCC CU UCAAGGACAUCGUCCGGG CCAUGGGG | 10816 |
| 3066 | CCCAUGGG G GACUGUUG | 2329 | CAACAGUC GGAGGAAACUCC CU UCAAGGACAUCGUCCGGG CCCAUGGG | 10817 |
| 3067 | CCAUGGGG G ACUGUUGG | 2330 | CCAACAGU GGAGGAAACUCC CU UCAAGGACAUCGUCCGGG CCCCAUGG | 10818 |
| 3074 | GGACUGUU G GGGUGGAG | 2331 | CUCCACCC GGAGGAAACUCC CU UCAAGGACAUCGUCCGGG AACAGUCC | 10819 |
| 3075 | GACUGUUG G GGUGGAGC | 2332 | GCUCCACC GGAGGAAACUCC CU UCAAGGACAUCGUCCGGG CAACAGUC | 10820 |
| 3076 | ACUGUUGG G GUGGAGCC | 2333 | GGCUCCAC GGAGGAAACUCC CU UCAAGGACAUCGUCCGGG CCAACAGU | 10821 |
| 3079 | GUUGGGGU G GAGCCCUC | 2334 | GAGGGCUC GGAGGAAACUCC CU UCAAGGACAUCGUCCGGG ACCCCAAC | 10822 |
| 3080 | UUGGGGUG G AGCCCUCA | 2335 | UGAGGGCU GGAGGAAACUCC CU UCAAGGACAUCGUCCGGG CACCCCAA | 10823 |
| 3095 | CACGCUCA G GGCCUACU | 2336 | AGUAGGCC GGAGGAAACUCC CU UCAAGGACAUCGUCCGGG UGAGCGUG | 10824 |
| 3096 | ACGCUCAG G GCCUACUC | 2337 | GAGUAGGC GGAGGAAACUCC CU UCAAGGACAUCGUCCGGG CUGAGCGU | 10825 |
| 3145 | CACCAAUC G GCAGUCAG | 2338 | CUGACUGC GGAGGAAACUCC CU UCAAGGACAUCGUCCGGG GAUUGGUG | 10826 |
| 3153 | GGCAGUCA G GAAGGCAG | 2339 | CUGCCUUC GGAGGAAACUCC CU UCAAGGACAUCGUCCGGG UGACUGCC | 10827 |
| 3154 | GCAGUCAG G AAGGCAGC | 2340 | GCUGCCUU GGAGGAAACUCC CU UCAAGGACAUCGUCCGGG CUGACUGC | 10828 |
| 3157 | GUCAGGAA G GCAGCCUA | 2341 | UAGGCUGC GGAGGAAACUCC CU UCAAGGACAUCGUCCGGG UUCCUGAC | 10829 |
| 3187 | ACCUCUAA G GGACACUC | 2342 | GAGUGUCC GGAGGAAACUCC CU UCAAGGACAUCGUCCGGG UUAGAGGU | 10830 |
| 3188 | CCUCUAAG G GACACUCA | 2343 | UGAGUGUC GGAGGAAACUCC CU UCAAGGACAUCGUCCGGG CUUAGAGG | 10831 |
| 3189 | CUCUAAGG G ACACUCAU | 2344 | AUGAGUGU GGAGGAAACUCC CU UCAAGGACAUCGUCCGGG CCUUAGAG | 10832 |
| 3203 | CAUCCUCA G GCCAUGCA | 2345 | UGCAUGGC GGAGGAAACUCC CU UCAAGGACAUCGUCCGGG UGAGGAUG | 10833 |

Input Sequence = AF100308.
Cut Site = YG/M or UG/U.
Stem Length = 8.
Core Sequence = GGAGGAAACUCC CU UCAAGGACAUCGUCCGGG
AF100308 (Hepatitis B virus strain 2-18, 3215 bp)

TABLE XI

| Human HBV Enzymatic Nucleic Acid and Target Sequence | | | | | | | |
|---|---|---|---|---|---|---|---|
| | | Seq | | | | Seq | |
| Pos | SUBSTRATE | ID | RPI# | Ribozyme Alias | ENZYMATIC NUCLEIC ACID | ID | |
| 313 | CCAAAAU U CGCAGUC | 2346 | 18157 | HBV-313 Rz-7 RNA | GACUGCG CUGAUGAGGCCGUUAGGCCGAA AUUUUGG B | 10834 | |
| 327 | CCCAAAU C UCCAGUC | 2347 | 18158 | HBV-327 Rz-7 RNA | GACUGGA CUGAUGAGGCCGUUAGGCCGAA AUUUGGG B | 10835 | |
| 334 | CUCCAGU C ACUCACC | 2348 | 18159 | HBV-334 Rz-7 RNA | GGUGAGU CUGAUGAGGCCGUUAGGCCGAA ACUGGAG B | 10836 | |
| 408 | UCUUCCU C UGCAUCC | 2349 | 18160 | HBV-408 Rz-7 RNA | GGAUGCA CUGAUGAGGCCGUUAGGCCGAA AGGAAGA B | 10837 | |
| 557 | UCUAUGU U UCCCUCA | 2350 | 18161 | HBV-557 Rz-7 RNA | UGAGGGA CUGAUGAGGCCGUUAGGCCGAA ACAUAGA B | 10838 | |
| 1255 | UUUGUGU C UCCUCUG | 2351 | 18162 | HBV-1255 Rz-7 RNA | CAGAGGA CUGAUGAGGCCGUUAGGCCGAA ACACAAA B | 10839 | |
| 1538 | CCUCUCU U UACGCGG | 2352 | 18163 | HBV-1538 Rz-7 RNA | CCGCGUA CUGAUGAGGCCGUUAGGCCGAA AGAGAGG B | 10840 | |
| 1756 | AGGAGGU U AGGUUAA | 2353 | 18164 | HBV-1756 Rz-7 RNA | UUAACCU CUGAUGAGGCCGUUAGGCCGAA ACCUCCU B | 10841 | |
| 1861 | AUGUCCU A CUGUUCA | 2354 | 18165 | HBV-1861 Rz-7 RNA | UGAACAG CUGAUGAGGCCGUUAGGCCGAA AGGACAU B | 10842 | |
| 2504 | UUCUUCU A CGGUACC | 2355 | 18166 | HBV-2504 Rz-7 RNA | GGUACCG CUGAUGAGGCCGUUAGGCCGAA AGAAGAA B | 10843 | |
| 10 | CUCCACC A CUUUCCA | 2356 | 18197 | HBV-10 CHz-7 RNA | UGGAAAG CUGAUGAGGCCGUUAGGCCGAA GGUGGAG B | 10844 | |
| 335 | UCCAGUC A CUCACCA | 2357 | 18198 | HBV-335 CHz-7 RNA | UGGUGAG CUGAUGAGGCCGUUAGGCCGAA GACUGGA B | 10845 | |
| 1258 | GUGUCUC C UCUGCCG | 2358 | 18199 | HBV-1258 CHz-7 RNA | CGGCAGA CUGAUGAGGCCGUUAGGCCGAA GAGACAC B | 10846 | |
| 2307 | GACCACC A AAUGCCC | 2359 | 18200 | HBV-2307 CHz-7 RNA | GGGCAUU CUGAUGAGGCCGUUAGGCCGAA GGUGGUC B | 10847 | |
| 347 | UCACCAACCU G UUGUC | 2360 | 18216 | HBV-347 GCl.Rz-5/10 RNA | GACAA UGAUGGCAUGCACUAUGCGCG AGGUUGGUGA B | 10848 | |
| 350 | CCAACCUGUU G UCCUC | 2361 | 18217 | HBV-350 GCl.Rz-5/10 RNA | GAGGA UGAUGGCAUGCACUAUGCGCG AACAGGUUGG B | 10849 | |
| 1508 | UCCGCCUAUU G UACCG | 2362 | 18218 | HBV-1508 GCl.Rz-5/10 RNA | CGGUA UGAUGGCAUGCACUAUGCGCG AAUAGGCGGA B | 10850 | |
| 234 | AAUCCU C ACAAUA | 2363 | 18334 | HBV-234 Rz-6 allyl stab1 | uₛaₛuₛuₛgu cUGAuGaggccguuaggccGaa Aggauu B | 10851 | |
| 252 | GAGUCU A GACUCG | 2364 | 18335 | HBV-252 Rz-6 allyl stab1 | cₛgₛaₛgₛuc cUGAuGaggccguuaggccGaa Agacuc B | 10852 | |
| 268 | UGGACU U CUCUCA | 2365 | 18337 | HBV-268 Rz-6 allyl stab1 | uₛgₛaₛgₛag cUGAuGaggccguuaggccGaa Agucca B | 10853 | |
| 280 | AAUUUU C UAGGGG | 2366 | 18345 | HBV-280 Rz-6 allyl stab1 | cₛcₛcₛcₛua cUGAuGaggccguuaggccGaa Aaaauu B | 10854 | |
| 313 | CAAAAU U CGCAGU | 2367 | 18346 | HBV-313 Rz-6 allyl stab1 | aₛcₛuₛgₛcg cUGAuGaggccguuaggccGaa Auuuug B | 10855 | |
| 395 | GGCGUU U UAUCAU | 2368 | 18350 | HBV-395 Rz-6 allyl stab1 | aₛuₛgₛaₛua cUGAuGaggccguuaggccGaa Aacgcc B | 10856 | |
| 402 | UAUCAU C UUCCUC | 2369 | 18351 | HBV-402 Rz-6 allyl stab1 | gₛaₛgₛgₛaa cUGAuGaggccguuaggccGaa Augaua B | 10857 | |
| 607 | UGUAUU C CCAUCC | 2370 | 18355 | HBV-607 Rz-6 allyl stab1 | gₛgₛaₛuₛgg cUGAuGaggccguuaggccGaa Aauaca B | 10858 | |
| 697 | UUUGUU C AGUGGU | 2371 | 18362 | HBV-697 Rz-6 allyl stab1 | aₛcₛcₛaₛcu cUGAuGaggccguuaggccGaa Aacaaa B | 10859 | |
| 1539 | UCUCUU U ACGCGG | 2372 | 18366 | HBV-1539 Rz-6 allyl stab1 | cₛcₛgₛcₛgu cUGAuGaggccguuaggccGaa Aagaga B | 10860 | |
| 1599 | UCACCU C UGCACG | 2373 | 18367 | HBV-1599 Rz-6 allyl stab1 | cₛgₛuₛgₛca cUGAuGaggccguuaggccGaa Agguga B | 10861 | |
| 1607 | GCACGU C GCAUGG | 2374 | 18368 | HBV-1607 Rz-6 allyl stab1 | cₛcₛaₛuₛgc cUGAuGaggccguuaggccGaa Acgugc B | 10862 | |
| 1833 | UCACCU C UGCCUA | 2375 | 18371 | HBV-1833 Rz-6 allyl stab1 | uₛaₛgₛgₛca cUGAuGaggccguuaggccGaa Agguga B | 10863 | |
| 2383 | AGAACU C CCUCGC | 2376 | 18374 | HBV-2383 Rz-6 allyl stab1 | gₛcₛgₛaₛgg cUGAuGaggccguuaggccGaa Aguucu B | 10864 | |
| 2429 | GAAGAU C UCAAUC | 2377 | 18376 | HBV-2429 Rz-6 allyl stab1 | gₛaₛuₛuₛga cUGAuGaggccguuaggccGaa Aucuuc B | 10865 | |
| 2831 | UAUUCU U GGGAAC | 2378 | 18379 | HBV-2831 Rz-6 allyl stab1 | gₛuₛuₛcₛcc cUGAuGaggccguuaggccGaa Agaaua B | 10866 | |
| 430 | UGCCUC A UCUUCU | 2379 | 18391 | HBV-430 CHz-6 allyl stab1 | aₛgₛaₛaₛga cUGAuGaggccguuaggccGaa Iaggca B | 10867 | |
| 676 | UGGCUC A GUUUAC | 2380 | 18396 | HBV-676 CHz-6 allyl stab1 | gₛuₛaₛaₛac cUGAuGaggccguuaggccGaa Iagcca B | 10868 | |
| 683 | GUUUAC U AGUGCC | 2381 | 18397 | HBV-683 CHz-6 allyl stab1 | gₛgₛcₛaₛcu cUGAuGaggccguuaggccGaa Iuaaac B | 10869 | |
| 1150 | UUUACC C CGUUGC | 2382 | 18402 | HBV-1150 CHz-6 allyl stab1 | gₛcₛaₛaₛcg cUGAuGaggccguuaggccGaa Iguaaa B | 10870 | |
| 1200 | GCAACC C CCACUG | 2383 | 18403 | HBV-1200 CHz-6 allyl stab1 | cₛaₛgₛuₛgg cUGAuGaggccguuaggccGaa Iguugc B | 10871 | |
| 1201 | CAACCC C CACUGG | 2384 | 18404 | HBV-1201 CHz-6 allyl stab1 | cₛcₛaₛgₛug cUGAuGaggccguuaggccGaa Igguug B | 10872 | |
| 1444 | CGGCGC U GAAUCC | 2385 | 18405 | HBV-1444 CHz-6 allyl stab1 | gₛgₛaₛuₛuc cUGAuGaggccguuaggccGaa Icgccg B | 10873 | |
| 1451 | GAAUCC C GCGGAC | 2386 | 18406 | HBV-1451 CHz-6 allyl stab1 | gₛuₛcₛcₛgc cUGAuGaggccguuaggccGaa Igauuc B | 10874 | |
| 1533 | CGCACC U CUCUUU | 2387 | 18407 | HBV-1533 CHz-6 allyl stab1 | aₛaₛaₛgₛag cUGAuGaggccguuaggccGaa Igugcg B | 10875 | |
| 1600 | CACCUC U GCACGU | 2388 | 18410 | HBV-1600 CHz-6 allyl stab1 | aₛcₛgₛuₛgc cUGAuGaggccguuaggccGaa Iaggug B | 10876 | |
| 1698 | CCGACC U UGAGGC | 2389 | 18411 | HBV-1698 CHz-6 allyl stab1 | gₛcₛcₛuₛca cUGAuGaggccguuaggccGaa Igucgg B | 10877 | |
| 1784 | GGAGGC U GUAGGC | 2390 | 18412 | HBV-1784 CHz-6 allyl stab1 | gₛcₛcₛuₛac cUGAuGaggccguuaggccGaa Iccucc B | 10878 | |
| 1829 | UUUUUC A CCUCUG | 2391 | 18414 | HBV-1829 CHz-6 allyl stab1 | cₛaₛgₛaₛgg cUGAuGaggccguuaggccGaa Iaaaaa B | 10879 | |
| 1876 | GCCUCC A AGCUGU | 2392 | 18420 | HBV-1876 CHz-6 allyl stab1 | aₛcₛaₛgₛcu cUGAuGaggccguuaggccGaa Igaggc B | 10880 | |
| 1880 | CCAAGC U GUGCCU | 2393 | 18422 | HBV-1880 CHz-6 allyl stab1 | aₛgₛgₛcₛac cUGAuGaggccguuaggccGaa Icuugg B | 10881 | |
| 218 | UUUUUCU U GUUGACA | 2394 | 18333 | HBV-218 Rz-7 allyl stab1 | uₛgₛuₛcₛaac cUGAuGaggccguuaggccGaa Agaaaaa B | 10882 | |
| 257 | CUAGACU C GUGGUGG | 2395 | 18336 | HBV-257 Rz-7 allyl stab1 | cₛcₛaₛcₛcac cUGAuGaggccguuaggccGaa Agucuag B | 10883 | |
| 268 | GUGGACU U CUCUCAA | 2396 | 18338 | HBV-268 Rz-7 allyl stab1 | uₛuₛgₛaₛgag cUGAuGaggccguuaggccGaa Aguccac B | 10884 | |
| 269 | UGGACUU C UCUCAAU | 2397 | 18339 | HBV-269 Rz-7 allyl stab1 | aₛuₛuₛgₛaga cUGAuGaggccguuaggccGaa Aagucca B | 10885 | |
| 271 | GACUUCU C UCAAUUU | 2398 | 18340 | HBV-271 Rz-7 allyl stab1 | aₛaₛaₛuₛuga cUGAuGaggccguuaggccGaa Agaaguc B | 10886 | |
| 273 | CUUCUCU C AAUUUUC | 2399 | 18341 | HBV-273 Rz-7 allyl stab1 | gₛaₛaₛaₛauu cUGAuGaggccguuaggccGaa Agagaag B | 10887 | |
| 277 | UCUCAAU U UUCUAGG | 2400 | 18342 | HBV-277 Rz-7 allyl stab1 | cₛcₛuₛaₛgaa cUGAuGaggccguuaggccGaa Auugaga B | 10888 | |
| 278 | CUCAAUU U UCUAGGG | 2401 | 18343 | HBV-278 Rz-7 allyl stab1 | cₛcₛcₛuₛaga cUGAuGaggccguuaggccGaa Aauugag B | 10889 | |
| 279 | UCAAUUU U CUAGGGG | 2402 | 18344 | HBV-279 Rz-7 allyl stab1 | cₛcₛcₛcₛuag cUGAuGaggccguuaggccGaa Aaauuga B | 10890 | |
| 314 | CAAAAUU C GCAGUCC | 2403 | 18347 | HBV-314 Rz-7 allyl stab1 | gₛgₛaₛcₛugc cUGAuGaggccguuaggccGaa Aauuuug B | 10891 | |
| 385 | GAUGUGU C UGCGGCG | 2404 | 18348 | HBV-385 Rz-7 allyl stab1 | cₛgₛcₛcₛgca cUGAuGaggccguuaggccGaa Acacauc B | 10892 | |
| 394 | GCGGCGU U UUAUCAU | 2405 | 18349 | HBV-394 Rz-7 allyl stab1 | aₛuₛgₛaₛuaa cUGAuGaggccguuaggccGaa Acgccgc B | 10893 | |
| 402 | UUAUCAU C UUCCUCU | 2406 | 18352 | HBV-402 Rz-7 allyl stab1 | aₛgₛaₛgₛgaa cUGAuGaggccguuaggccGaa Augauaa B | 10894 | |
| 423 | UGCUGCU A UGCCUCA | 2407 | 18353 | HBV-423 Rz-7 allyl stab1 | uₛgₛaₛgₛgca cUGAuGaggccguuaggccGaa Agcagca B | 10895 | |
| 429 | UAUGCCU C AUCUUCU | 2408 | 18354 | HBV-429 Rz-7 allyl stab1 | aₛgₛaₛaₛgau cUGAuGaggccguuaggccGaa Aggcaua B | 10896 | |
| 679 | GCUCAGU U UACUAGU | 2409 | 18356 | HBV-679 Rz-7 allyl stab1 | aₛcₛuₛaₛgua cUGAuGaggccguuaggccGaa Acugagc B | 10897 | |
| 680 | CUCAGUU U ACUAGUG | 2410 | 18357 | HBV-680 Rz-7 allyl stab1 | cₛaₛcₛuₛagu cUGAuGaggccguuaggccGaa Aacugag B | 10898 | |
| 681 | UCAGUUU A CUAGUGC | 2411 | 18358 | HBV-681 Rz-7 allyl stab1 | gₛcₛaₛcₛuag cUGAuGaggccguuaggccGaa Aaacuga B | 10899 | |
| 684 | GUUUACU A GUGCCAU | 2412 | 18359 | HBV-684 Rz-7 allyl stab1 | aₛuₛgₛgₛcac cUGAuGaggccguuaggccGaa Aguaaac B | 10900 | |
| 692 | GUGCCAU U UGUUCAG | 2413 | 18360 | HBV-692 Rz-7 allyl stab1 | cₛuₛgₛaₛaca cUGAuGaggccguuaggccGaa Auggcac B | 10901 | |
| 693 | UGCCAUU U GUUCAGU | 2414 | 18361 | HBV-693 Rz-7 allyl stab1 | aₛcₛuₛgₛaac cUGAuGaggccguuaggccGaa Aauggca B | 10902 | |
| 1534 | CGCACCU C UCUUUAC | 2415 | 18363 | HBV-1534 Rz-7 allyl stab1 | gₛuₛaₛaₛaga cUGAuGaggccguuaggccGaa Aggugcg B | 10903 | |
| 1536 | CACCUCU C UUUACGC | 2416 | 18364 | HBV-1536 Rz-7 allyl stab1 | gₛcₛgₛuₛaaa cUGAuGaggccguuaggccGaa Agaggug B | 10904 | |
| 1538 | CCUCUCU U UACGCGG | 2352 | 18365 | HBV-1538 Rz-7 allyl stab1 | cₛcₛgₛcₛgua cUGAuGaggccguuaggccGaa Agagagg B | 10905 | |
| 1787 | AGGCUGU A GGCAUAA | 2417 | 18369 | HBV-1787 Rz-7 allyl stab1 | uₛuₛaₛuₛgcc cUGAuGaggccguuaggccGaa Acagccu B | 10906 | |
| 1793 | UAGGCAU A AAUUGGU | 2418 | 18370 | HBV-1793 Rz-7 allyl stab1 | aₛcₛcₛaₛauu cUGAuGaggccguuaggccGaa Augccua B | 10907 | |
| 1874 | CAAGCCU C CAAGCUG | 2419 | 18372 | HBV-1874 Rz-7 allyl stab1 | cₛaₛgₛcₛuug cUGAuGaggccguuaggccGaa Aggcuug B | 10908 | |
| 1887 | UGUGCCU U GGGUGGC | 2420 | 18373 | HBV-1887 Rz-7 allyl stab1 | gₛcₛcₛaₛccc cUGAuGaggccguuaggccGaa Aggcaca B | 10909 | |
| 2383 | AAGAACU C CCUCGCC | 2421 | 18375 | HBV-2383 Rz-7 allyl stab1 | gₛgₛcₛgₛagg cUGAuGaggccguuaggccGaa Aguucuu B | 10910 | |
| 2828 | ACCAUAU U CUUGGGA | 2422 | 18377 | HBV-2828 Rz-7 allyl stab1 | uₛcₛcₛcₛaag cUGAuGaggccguuaggccGaa Auauggu B | 10911 | |
| 2829 | CCAUAUU C UUGGGAA | 2423 | 18378 | HBV-2829 Rz-7 allyl stab1 | uₛuₛcₛcₛcaa cUGAuGaggccguuaggccGaa Aauaugg B | 10912 | |
| 2831 | AUAUUCU U GGGAACA | 2424 | 18380 | HBV-2831 Rz-7 allyl stab1 | uₛgₛuₛuₛccc cUGAuGaggccguuaggccGaa Agaauau B | 10913 | |
| 256 | UCUAGAC U CGUGGUG | 2425 | 18381 | HBV-256 CHz-7 allyl stab1 | cₛaₛcₛcₛacg cUGAuGaggccguuaggccGaa Iucuaga B | 10914 | |
| 267 | GGUGGAC U UCUCUCA | 2426 | 18382 | HBV-267 CHz-7 allyl stab1 | uₛgₛaₛgₛaga cUGAuGaggccguuaggccGaa Iuccacc B | 10915 | |
| 270 | GGACUUC U CUCAAUU | 2427 | 18383 | HBV-270 CHz-7 allyl stab1 | aₛaₛuₛuₛgag cUGAuGaggccguuaggccGaa Iaagucc B | 10916 | |
| 272 | ACUUCUC U CAAUUUU | 2428 | 18384 | HBV-272 CHz-7 allyl stab1 | aₛaₛaₛaₛuug cUGAuGaggccguuaggccGaa Iagaagu B | 10917 | |
| 274 | UUCUCUC A AUUUUCU | 2429 | 18385 | HBV-274 CHz-7 allyl stab1 | aₛgₛaₛaₛaau cUGAuGaggccguuaggccGaa Iagagaa B | 10918 | |
| 386 | AUGUGUC U GCGGCGU | 2430 | 18386 | HBV-386 CHz-7 allyl stab1 | aₛcₛgₛcₛcgc cUGAuGaggccguuaggccGaa Iacacau B | 10919 | |
| 419 | AUCCUGC U GCUAUGC | 2431 | 18387 | HBV-419 CHz-7 allyl stab1 | gₛcₛaₛuₛagc cUGAuGaggccguuaggccGaa Icaggau B | 10920 | |
| 422 | CUGCUGC U AUGCCUC | 2432 | 18388 | HBV-422 CHz-7 allyl stab1 | gₛaₛgₛgₛcau cUGAuGaggccguuaggccGaa Icagcag B | 10921 | |
| 427 | GCUAUGC C UCAUCUU | 2433 | 18389 | HBV-427 CHz-7 allyl stab1 | aₛaₛgₛaₛuga cUGAuGaggccguuaggccGaa Icauagc B | 10922 | |
| 428 | CUAUGCC U CAUCUUC | 2434 | 18390 | HBV-428 CHz-7 allyl stab1 | gₛaₛaₛgₛaug cUGAuGaggccguuaggccGaa Igcauag B | 10923 | |
| 430 | AUGCCUC A UCUUCUU | 2435 | 18392 | HBV-430 CHz-7 allyl stab1 | aₛaₛgₛaₛaga cUGAuGaggccguuaggccGaa Iaggcau B | 10924 | |
| 608 | UGUAUUC C CAUCCCA | 2436 | 18393 | HBV-608 CHz-7 allyl stab1 | uₛgₛgₛgₛaug cUGAuGaggccguuaggccGaa Iaauaca B | 10925 | |
| 609 | GUAUUCC C AUCCCAU | 2437 | 18394 | HBV-609 CHz-7 allyl stab1 | aₛuₛgₛgₛgau cUGAuGaggccguuaggccGaa Igaauac B | 10926 | |
| 669 | GUUUCUC U UGGCUCA | 2438 | 18395 | HBV-669 CHz-7 allyl stab1 | uₛgₛaₛgₛcca cUGAuGaggccguuaggccGaa Iagaaac B | 10927 | |
| 689 | CUAGUGC C AUUUGUU | 2439 | 18398 | HBV-689 CHz-7 allyl stab1 | aₛaₛcₛaₛaau cUGAuGaggccguuaggccGaa Icacuag B | 10928 | |
| 690 | UAGUGCC A UUUGUUC | 2440 | 18399 | HBV-690 CHz-7 allyl stab1 | gₛaₛaₛcₛaaa cUGAuGaggccguuaggccGaa Igcacua B | 10929 | |
| 718 | GCUUUCC C CCACUGU | 2441 | 18400 | HBV-718 CHz-7 allyl stab1 | aₛcₛaₛgₛugg cUGAuGaggccguuaggccGaa Igaaagc B | 10930 | |
| 1149 | CCUUUAC C CCGUUGC | 2442 | 18401 | HBV-1149 CHz-7 allyl stab1 | gₛcₛaₛaₛcgg cUGAuGaggccguuaggccGaa Iuaaagg B | 10931 | |
| 1535 | GCACCUC U CUUUACG | 2443 | 18408 | HBV-1535 CHz-7 allyl stab1 | cₛgₛuₛaₛaag cUGAuGaggccguuaggccGaa Iaggugc B | 10932 | |
| 1537 | ACCUCUC U UUACGCG | 2444 | 18409 | HBV-1537 CHz-7 allyl stab1 | cₛgₛcₛgₛuaa cUGAuGaggccguuaggccGaa Iagaggu B | 10933 | |
| 1791 | UGUAGGC A UAAAUUG | 2445 | 18413 | HBV-1791 CHz-7 allyl stab1 | cₛaₛaₛuₛuua cUGAuGaggccguuaggccGaa Iccuaca B | 10934 | |
| 1831 | UUUUCAC C UCUGCCU | 2446 | 18415 | HBV-1831 CHz-7 allyl stab1 | aₛgₛgₛcₛaga cUGAuGaggccguuaggccGaa Iugaaaa B | 10935 | |
| 1832 | UUUCACC U CUGCCUA | 2447 | 18416 | HBV-1832 CHz-7 allyl stab1 | uₛaₛgₛgₛcag cUGAuGaggccguuaggccGaa Igugaaa B | 10936 | |
| 1872 | UUCAAGC C UCCAAGC | 2448 | 18417 | HBV-1872 CHz-7 allyl stab1 | gₛcₛuₛuₛgga cUGAuGaggccguuaggccGaa Icuugaa B | 10937 | |
| 1873 | UCAAGCC U CCAAGCU | 2449 | 18418 | HBV-1873 CHz-7 allyl stab1 | aₛgₛcₛuₛugg cUGAuGaggccguuaggccGaa Igcuuga B | 10938 | |
| 1875 | AAGCCUC C AAGCUGU | 2450 | 18419 | HBV-1875 CHz-7 allyl stab1 | aₛcₛaₛgₛcuu cUGAuGaggccguuaggccGaa Iaggcuu B | 10939 | |
| 1876 | AGCCUCC A AGCUGUG | 2451 | 18421 | HBV-1876 CHz-7 allyl stab1 | cₛaₛcₛaₛgcu cUGAuGaggccguuaggccGaa Igaggcu B | 10940 | |
| 1880 | UCCAAGC U GUGCCUU | 2452 | 18423 | HBV-1880 CHz-7 allyl stab1 | aₛaₛgₛgₛcac cUGAuGaggccguuaggccGaa Icuugga B | 10941 | |
| 2382 | GAAGAAC U CCCUCGC | 2453 | 18424 | HBV-2382 CHz-7 allyl stab1 | gₛcₛgₛaₛggg cUGAuGaggccguuaggccGaa Iuucuuc B | 10942 | |
| 2384 | AGAACUC C CUCGCCU | 2454 | 18425 | HBV-2384 CHz-7 allyl stab1 | aₛgₛgₛcₛgag cUGAuGaggccguuaggccGaa Iaguucu B | 10943 | |
| 2385 | GAACUCC C UCGCCUC | 2455 | 18426 | HBV-2385 CHz-7 allyl stab1 | gₛaₛgₛgₛcga cUGAuGaggccguuaggccGaa Igaguuc B | 10944 | |
| 2422 | GCGUCGC A GAAGAUC | 2456 | 18427 | HBV-2422 CHz-7 allyl stab1 | gₛaₛuₛcₛuuc cUGAuGaggccguuaggccGaa Icgacgc B | 10945 | |
| 2830 | CAUAUUC U UGGGAAC | 2457 | 18428 | HBV-2830 CHz-7 allyl stab1 | gₛuₛuₛcₛcca cUGAuGaggccguuaggccGaa Iaauaug B | 10946 | |
| 234 | AAUCCU C ACAAUA | 2363 | 19179 | HBV-234 Rz-6 amino stab1 | uₛaₛuₛuₛgu cUGAUGaggccguuaggccGaa Aggauu B | 10947 | |
| 252 | GAGUCU A GACUCG | 2364 | 19180 | HBV-252 Rz-6 amino stab1 | cₛgₛaₛgₛuc cUGAUGaggccguuaggccGaa Agacuc B | 10948 | |
| 268 | UGGACU U CUCUCA | 2365 | 19182 | HBV-268 Rz-6 amino stab1 | uₛgₛaₛgₛag cUGAUGaggccguuaggccGaa Agucca B | 10949 | |
| 280 | AAUUUU C UAGGGG | 2366 | 19190 | HBV-280 Rz-6 amino stab1 | cₛcₛcₛcₛua cUGAUGaggccguuaggccGaa Aaaauu B | 10950 | |
| 313 | CAAAAU U CGCAGU | 2367 | 19191 | HBV-313 Rz-6 amino stab1 | aₛcₛuₛgₛcg cUGAUGaggccguuaggccGaa Auuuug B | 10951 | |
| 395 | GGCGUU U UAUCAU | 2368 | 19195 | HBV-395 Rz-6 amino stab1 | aₛuₛgₛaₛua cUGAUGaggccguuaggccGaa Aacgcc B | 10952 | |
| 402 | UAUCAU C UUCCUC | 2369 | 19196 | HBV-402 Rz-6 amino stab1 | gₛaₛgₛgₛaa cUGAUGaggccguuaggccGaa Augaua B | 10953 | |
| 607 | UGUAUU C CCAUCC | 2370 | 19200 | HBV-607 Rz-6 amino stab1 | gₛgₛaₛuₛgg cUGAUGaggccguuaggccGaa Aauaca B | 10954 | |
| 697 | UUUGUU C AGUGGU | 2371 | 19207 | HBV-697 Rz-6 amino stab1 | aₛcₛcₛaₛcu cUGAUGaggccguuaggccGaa Aacaaa B | 10955 | |
| 1539 | UCUCUU U ACGCGG | 2372 | 19211 | HBV-1539 Rz-6 amino stab1 | cₛcₛgₛcₛgu cUGAUGaggccguuaggccGaa Aagaga B | 10956 | |
| 1599 | UCACCU C UGCACG | 2373 | 19212 | HBV-1599 Rz-6 amino stab1 | cₛgₛuₛgₛca cUGAUGaggccguuaggccGaa Agguga B | 10957 | |
| 1607 | GCACGU C GCAUGG | 2374 | 19213 | HBV-1607 Rz-6 amino stab1 | cₛcₛaₛuₛgc cUGAUGaggccguuaggccGaa Acgugc B | 10958 | |
| 1833 | UCACCU C UGCCUA | 2375 | 19216 | HBV-1833 Rz-6 amino stab1 | uₛaₛgₛgₛca cUGAUGaggccguuaggccGaa Agguga B | 10959 | |
| 2383 | AGAACU C CCUCGC | 2376 | 19219 | HBV-2383 Rz-6 amino stab1 | gₛcₛgₛaₛgg cUGAUGaggccguuaggccGaa Aguucu B | 10960 | |
| 2429 | GAAGAU C UCAAUC | 2377 | 19221 | HBV-2429 Rz-6 amino stab1 | gₛaₛuₛuₛga cUGAUGaggccguuaggccGaa Aucuuc B | 10961 | |
| 2831 | UAUUCU U GGGAAC | 2378 | 19224 | HBV-2831 Rz-6 amino stab1 | gₛuₛuₛcₛcc cUGAUGaggccguuaggccGaa Agaaua B | 10962 | |
| 430 | UGCCUC A UCUUCU | 2379 | 19236 | HBV-430 CHz-6 amino stab1 | aₛgₛaₛaₛga cUGAUGaggccguuaggccGaa Iaggca B | 10963 | |
| 676 | UGGCUC A GUUUAC | 2380 | 19241 | HBV-676 CHz-6 amino stab1 | gₛuₛaₛaₛac cUGAUGaggccguuaggccGaa Iagcca B | 10964 | |
| 683 | GUUUAC U AGUGCC | 2381 | 19242 | HBV-683 CHz-6 amino stab1 | gₛgₛcₛaₛcu cUGAUGaggccguuaggccGaa Iuaaac B | 10965 | |
| 1150 | UUUACC C CGUUGC | 2382 | 19247 | HBV-1150 CHz-6 amino stab1 | gₛcₛaₛaₛcg cUGAUGaggccguuaggccGaa Iguaaa B | 10966 | |
| 1200 | GCAACC C CCACUG | 2383 | 19248 | HBV-1200 CHz-6 amino stab1 | cₛaₛgₛuₛgg cUGAUGaggccguuaggccGaa Iguugc B | 10967 | |
| 1201 | CAACCC C CACUGG | 2384 | 19249 | HBV-1201 CHz-6 amino stab1 | cₛcₛaₛgₛug cUGAUGaggccguuaggccGaa Igguug B | 10968 | |
| 1444 | CGGCGC U GAAUCC | 2385 | 19250 | HBV-1444 CHz-6 amino stab1 | gₛgₛaₛuₛuc cUGAUGaggccguuaggccGaa Icgccg B | 10969 | |
| 1451 | GAAUCC C GCGGAC | 2386 | 19251 | HBV-1451 CHz-6 amino stab1 | gₛuₛcₛcₛgc cUGAUGaggccguuaggccGaa Igauuc B | 10970 | |
| 1533 | CGCACC U CUCUUU | 2387 | 19252 | HBV-1533 CHz-6 amino stab1 | aₛaₛaₛgₛag cUGAUGaggccguuaggccGaa Igugcg B | 10971 | |
| 1600 | CACCUC U GCACGU | 2388 | 19255 | HBV-1600 CHz-6 amino stab1 | aₛcₛgₛuₛgc cUGAUGaggccguuaggccGaa Iaggug B | 10972 | |
| 1698 | CCGACC U UGAGGC | 2389 | 19256 | HBV-1698 CHz-6 amino stab1 | gₛcₛcₛuₛca cUGAUGaggccguuaggccGaa Igucgg B | 10973 | |
| 1784 | GGAGGC U GUAGGC | 2390 | 19257 | HBV-1784 CHz-6 amino stab1 | gₛcₛcₛuₛac cUGAUGaggccguuaggccGaa Iccucc B | 10974 | |
| 1829 | UUUUUC A CCUCUG | 2391 | 19259 | HBV-1829 CHz-6 amino stab1 | cₛaₛgₛaₛgg cUGAUGaggccguuaggccGaa Iaaaaa B | 10975 | |
| 1876 | GCCUCC A AGCUGU | 2392 | 19265 | HBV-1876 CHz-6 amino stab1 | aₛcₛaₛgₛcu cUGAUGaggccguuaggccGaa Igaggc B | 10976 | |
| 1880 | CCAAGC U GUGCCU | 2393 | 19267 | HBV-1880 CHz-6 amino stab1 | aₛgₛgₛcₛac cUGAUGaggccguuaggccGaa Icuugg B | 10977 | |
| 218 | UUUUCU U GUUGACA | 2394 | 19178 | HBV-218 Rz-7 amino stab1 | uₛgₛuₛcₛaac cUGAUGaggccguuaggccGaa Agaaaaa B | 10978 | |
| 257 | CUAGACU C GUGGUGG | 2395 | 19181 | HBV-257 Rz-7 amino stab1 | cₛcₛaₛcₛcac cUGAUGaggccguuaggccGaa Agucuag B | 10979 | |
| 268 | GUGGACU U CUCUCAA | 2396 | 19183 | HBV-268 Rz-7 amino stab1 | uₛuₛgₛaₛgag cUGAUGaggccguuaggccGaa Aguccac B | 10980 | |
| 269 | UGGACUU C UCUCAAU | 2397 | 19184 | HBV-269 Rz-7 amino stab1 | aₛuₛuₛgₛaga cUGAUGaggccguuaggccGaa Aagucca B | 10981 | |
| 271 | GACUUCU C UCAAUUU | 2398 | 19185 | HBV-271 Rz-7 amino stab1 | aₛaₛaₛuₛuga cUGAUGaggccguuaggccGaa Agaaguc B | 10982 | |
| 273 | CUUCUCU C AAUUUUC | 2399 | 19186 | HBV-273 Rz-7 amino stab1 | gₛaₛaₛaₛauu cUGAUGaggccguuaggccGaa Agagaag B | 10983 | |
| 277 | UCUCAAU U UUCUAGG | 2400 | 19187 | HBV-277 Rz-7 amino stab1 | cₛcₛuₛaₛgaa cUGAUGaggccguuaggccGaa Auugaga B | 10984 | |
| 278 | CUCAAUU U UCUAGGG | 2401 | 19188 | HBV-278 Rz-7 amino stab1 | cₛcₛcₛuₛaga cUGAUGaggccguuaggccGaa Aauugag B | 10985 | |
| 279 | UCAAUUU U CUAGGGG | 2402 | 19189 | HBV-279 Rz-7 amino stab1 | cₛcₛcₛcₛuag cUGAUGaggccguuaggccGaa Aaauuga B | 10986 | |
| 314 | CAAAAUU C GCAGUCC | 2403 | 19192 | HBV-314 Rz-7 amino stab1 | gₛgₛaₛcₛugc cUGAUGaggccguuaggccGaa Aauuuug B | 10987 | |
| 385 | GAUGUGU C UGCGGCG | 2404 | 19193 | HBV-385 Rz-7 amino stab1 | cₛgₛcₛcₛgca cUGAUGaggccguuaggccGaa Acacauc B | 10988 | |
| 394 | GCGGCGU U UUAUCAU | 2405 | 19194 | HBV-394 Rz-7 amino stab1 | aₛuₛgₛaₛuaa cUGAUGaggccguuaggccGaa Acgccgc B | 10989 | |
| 402 | UUAUCAU C UUCCUCU | 2406 | 19197 | HBV-402 Rz-7 amino stab1 | aₛgₛaₛgₛgaa cUGAUGaggccguuaggccGaa Augauaa B | 10990 | |
| 423 | UGCUGCU A UGCCUCA | 2407 | 19198 | HBV-423 Rz-7 amino stab1 | uₛgₛaₛgₛgca cUGAUGaggccguuaggccGaa Agcagca B | 10991 | |
| 429 | UAUGCCU C AUCUUCU | 2408 | 19199 | HBV-429 Rz-7 amino stab1 | aₛgₛaₛaₛgau cUGAUGaggccguuaggccGaa Aggcaua B | 10992 | |
| 679 | GCUCAGU U UACUAGU | 2409 | 19201 | HBV-679 Rz-7 amino stab1 | aₛcₛuₛaₛgua cUGAUGaggccguuaggccGaa Acugagc B | 10993 | |
| 680 | CUCAGUU U ACUAGUG | 2410 | 19202 | HBV-680 Rz-7 amino stab1 | cₛaₛcₛuₛagu cUGAUGaggccguuaggccGaa Aacugag B | 10994 | |
| 681 | UCAGUUU A CUAGUGC | 2411 | 19203 | HBV-681 Rz-7 amino stab1 | gₛcₛaₛcₛuag cUGAUGaggccguuaggccGaa Aaacuga B | 10995 | |
| 684 | GUUUACU A GUGCCAU | 2412 | 19204 | HBV-684 Rz-7 amino stab1 | aₛuₛgₛgₛcac cUGAUGaggccguuaggccGaa Aguaaac B | 10996 | |
| 692 | GUGCCAU U UGUUCAG | 2413 | 19205 | HBV-692 Rz-7 amino stab1 | cₛuₛgₛaₛaca cUGAUGaggccguuaggccGaa Auggcac B | 10997 | |
| 693 | UGCCAUU U GUUCAGU | 2414 | 19206 | HBV-693 Rz-7 amino stab1 | aₛcₛuₛgₛaac cUGAUGaggccguuaggccGaa Aauggca B | 10998 | |
| 1534 | CGCACCU C UCUUUAC | 2415 | 19208 | HBV-1534 Rz-7 amino stab1 | gₛuₛaₛaₛaga cUGAUGaggccguuaggccGaa Aggugcg B | 10999 | |
| 1536 | CACCUCU C UUUACGC | 2416 | 19209 | HBV-1536 Rz-7 amino stab1 | gₛcₛgₛuₛaaa cUGAUGaggccguuaggccGaa Agaggug B | 11000 | |
| 1538 | CCUCUCU U UACGCGG | 2352 | 19210 | HBV-1538 Rz-7 amino stab1 | cₛcₛgₛcₛgua cUGAUGaggccguuaggccGaa Agagagg B | 11001 | |
| 1787 | AGGCUGU A GGCAUAA | 2417 | 19214 | HBV-1787 Rz-7 amino stab1 | uₛuₛaₛuₛgcc cUGAUGaggccguuaggccGaa Acagccu B | 11002 | |
| 1793 | UAGGCAU A AAUUGGU | 2418 | 19215 | HBV-1793 Rz-7 amino stab1 | aₛcₛcₛaₛauu cUGAUGaggccguuaggccGaa Augccua B | 11003 | |
| 1874 | CAAGCCU C CAAGCUG | 2419 | 19217 | HBV-1874 Rz-7 amino stab1 | cₛaₛgₛcₛuug cUGAUGaggccguuaggccGaa Aggcuug B | 11004 | |
| 1887 | UGUGCCU U GGGUGGC | 2420 | 19218 | HBV-1887 Rz-7 amino stab1 | gₛcₛcₛaₛccc cUGAUGaggccguuaggccGaa Aggcaca B | 11005 | |
| 2383 | AAGAACU C CCUCGCC | 2421 | 19220 | HBV-2383 Rz-7 amino stab1 | gₛgₛcₛgₛagg cUGAUGaggccguuaggccGaa Aguucuu B | 11006 | |
| 2828 | ACCAUAU U CUUGGGA | 2422 | 19222 | HBV-2828 Rz-7 amino stab1 | uₛcₛcₛcₛaag cUGAUGaggccguuaggccGaa Auauggu B | 11007 | |
| 2829 | CCAUAUU C UUGGGAA | 2423 | 19223 | HBV-2829 Rz-7 amino stab1 | uₛuₛcₛcₛcaa cUGAUGaggccguuaggccGaa Aauaugg B | 11008 | |
| 2831 | AUAUUCU U GGGAACA | 2424 | 19225 | HBV-2831 Rz-7 amino stab1 | uₛgₛuₛuₛccc cUGAUGaggccguuaggccGaa Agaauau B | 11009 | |
| 256 | UCUAGAC U CGUGGUG | 2425 | 19226 | HBV-256 CHz-7 amino stab1 | cₛaₛcₛcₛacg cUGAUGaggccguuaggccGaa Iucuaga B | 11010 | |
| 267 | GGUGGAC U UCUCUCA | 2426 | 19227 | HBV-267 CHz-7 amino stab1 | uₛgₛaₛgₛaga cUGAUGaggccguuaggccGaa Iuccacc B | 11011 | |
| 270 | GGACUUC U CUCAAUU | 2427 | 19228 | HBV-270 CHz-7 amino stab1 | aₛaₛuₛuₛgag cUGAUGaggccguuaggccGaa Iaagucc B | 11012 | |
| 272 | ACUUCUC U CAAUUUU | 2428 | 19229 | HBV-272 CHz-7 amino stab1 | aₛaₛaₛaₛuug cUGAUGaggccguuaggccGaa Iagaagu B | 11013 | |
| 274 | UUCUCUC A AUUUUCU | 2429 | 19230 | HBV-274 CHz-7 amino stab1 | aₛgₛaₛaₛaau cUGAUGaggccguuaggccGaa Iagagaa B | 11014 | |
| 386 | AUGUGUC U GCGGCGU | 2430 | 19231 | HBV-386 CHz-7 amino stab1 | aₛcₛgₛcₛcgc cUGAUGaggccguuaggccGaa Iacacau B | 11015 | |
| 419 | AUCCUGC U GCUAUGC | 2431 | 19232 | HBV-419 CHz-7 amino stab1 | gₛcₛaₛuₛagc cUGAUGaggccguuaggccGaa Icaggau B | 11016 | |
| 422 | CUGCUGC U AUGCCUC | 2432 | 19233 | HBV-422 CHz-7 amino stab1 | gₛaₛgₛgₛcau cUGAUGaggccguuaggccGaa Icagcag B | 11017 | |
| 427 | GCUAUGC C UCAUCUU | 2433 | 19234 | HBV-427 CHz-7 amino stab1 | aₛaₛgₛaₛuga cUGAUGaggccguuaggccGaa Icauagc B | 11018 | |
| 428 | CUAUGCC U CAUCUUC | 2434 | 19235 | HBV-428 CHz-7 amino stab1 | gₛaₛaₛgₛaug cUGAUGaggccguuaggccGaa Igcauag B | 11019 | |
| 430 | AUGCCUC A UCUUCUU | 2435 | 19237 | HBV-430 CHz-7 amino stab1 | aₛaₛgₛaₛaga cUGAUGaggccguuaggccGaa Iaggcau B | 11020 | |
| 608 | UGUAUUC C CAUCCCA | 2436 | 19238 | HBV-608 CHz-7 amino stab1 | uₛgₛgₛgₛaug cUGAUGaggccguuaggccGaa Iaauaca B | 11021 | |
| 609 | GUAUUCC C AUCCCAU | 2437 | 19239 | HBV-609 CHz-7 amino stab1 | aₛuₛgₛgₛgau cUGAUGaggccguuaggccGaa Igaauac B | 11022 | |
| 669 | GUUUCUC U UGGCUCA | 2438 | 19240 | HBV-669 CHz-7 amino stab1 | uₛgₛaₛgₛcca cUGAUGaggccguuaggccGaa Iagaaac B | 11023 | |
| 689 | CUAGUGC C AUUUGUU | 2439 | 19243 | HBV-689 CHz-7 amino stab1 | aₛaₛcₛaₛaau cUGAUGaggccguuaggccGaa Icacuag B | 11024 | |
| 690 | UAGUGCC A UUUGUUC | 2440 | 19244 | HBV-690 CHz-7 amino stab1 | gₛaₛaₛcₛaaa cUGAUGaggccguuaggccGaa Igcacua B | 11025 | |
| 718 | GCUUUCC C CCACUGU | 2441 | 19245 | HBV-718 CHz-7 amino stab1 | aₛcₛaₛgₛugg cUGAUGaggccguuaggccGaa Igaaagc B | 11026 | |
| 1149 | CCUUUAC C CCGUUGC | 2442 | 19246 | HBV-1149 CHz-7 amino stab1 | gₛcₛaₛaₛcgg cUGAUGaggccguuaggccGaa Iuaaagg B | 11027 | |
| 1535 | GCACCUC U CUUUACG | 2443 | 19253 | HBV-1535 CHz-7 amino stab1 | cₛgₛuₛaₛaag cUGAUGaggccguuaggccGaa Iaggugc B | 11028 | |
| 1537 | ACCUCUC U UUACGCG | 2444 | 19254 | HBV-1537 CHz-7 amino stab1 | cₛgₛcₛgₛuaa cUGAUGaggccguuaggccGaa Iagaggu B | 11029 | |
| 1791 | UGUAGGC A UAAAUUG | 2445 | 19258 | HBV-1791 CHz-7 amino stab1 | cₛaₛaₛuₛuua cUGAUGaggccguuaggccGaa Iccuaca B | 11030 | |
| 1831 | UUUUCAC C UCUGCCU | 2446 | 19260 | HBV-1831 CHz-7 amino stab1 | aₛgₛgₛcₛaga cUGAUGaggccguuaggccGaa Iugaaaa B | 11031 | |
| 1832 | UUUCACC U CUGCCUA | 2447 | 19261 | HBV-1832 CHz-7 amino stab1 | uₛaₛgₛgₛcag cUGAUGaggccguuaggccGaa Igugaaa B | 11032 | |
| 1872 | UUCAAGC C UCCAAGC | 2448 | 19262 | HBV-1872 CHz-7 amino stab1 | gₛcₛuₛuₛgga cUGAUGaggccguuaggccGaa Icuugaa B | 11033 | |
| 1873 | UCAAGCC U CCAAGCU | 2449 | 19263 | HBV-1873 CHz-7 amino stab1 | aₛgₛcₛuₛugg cUGAUGaggccguuaggccGaa Igcuuga B | 11034 | |
| 1875 | AAGCCUC C AAGCUGU | 2450 | 19264 | HBV-1875 CHz-7 amino stab1 | aₛcₛaₛgₛcuu cUGAUGaggccguuaggccGaa Iaggcuu B | 11035 | |
| 1876 | AGCCUCC A AGCUGUG | 2451 | 19266 | HBV-1876 CHz-7 amino stab1 | cₛaₛcₛaₛgcu cUGAUGaggccguuaggccGaa Igaggcu B | 11036 | |
| 1880 | UCCAAGC U GUGCCUU | 2452 | 19268 | HBV-1880 CHz-7 amino stab1 | aₛaₛgₛgₛcac cUGAUGaggccguuaggccGaa Icuugga B | 11037 | |
| 2382 | GAAGAAC U CCCUCGC | 2453 | 19269 | HBV-2382 CHz-7 amino stab1 | gₛcₛgₛaₛggg cUGAUGaggccguuaggccGaa Iuucuuc B | 11038 | |
| 2384 | AGAACUC C CUCGCCU | 2454 | 19270 | HBV-2384 CHz-7 amino stab1 | aₛgₛgₛcₛgag cUGAUGaggccguuaggccGaa Iaguucu B | 11039 | |
| 2385 | GAACUCC C UCGCCUC | 2455 | 19271 | HBV-2385 CHz-7 amino stab1 | gₛaₛgₛgₛcga cUGAUGaggccguuaggccGaa Igaguuc B | 11040 | |
| 2422 | GCGUCGC A GAAGAUC | 2456 | 19272 | HBV-2422 CHz-7 amino stab1 | gₛaₛuₛcₛuuc cUGAUGaggccguuaggccGaa Icgacgc B | 11041 | |
| 2830 | CAUAUUC U UGGGAAC | 2457 | 19273 | HBV-2830 CHz-7 amino stab1 | gₛuₛuₛcₛcca cUGAUGaggccguuaggccGaa Iaauaug B | 11042 | |
| 315 | GCCAAAAUUC G CAGUC | 2458 | 20079 | HBV-315 GCl.Rz-5/10 stab2 | gₛaₛcₛg uGAUₛg gcauGcacuaugc gcg gaauuuuggc B | 11043 | |
| 381 | AUCGCUGGAU G UGUCU | 2459 | 20080 | HBV-381 GCl.Rz-5/10 stab2 | aₛgₛaₛa uGAUₛg gcauGcacuaugc gcg auccagcgau B | 11044 | |
| 476 | UUGCCCGUUU G UCCUC | 2460 | 20081 | HBV-476 GCl.Rz-5/10 stab2 | gₛaₛgₛa uGAUₛg gcauGcacuaugc gcg aaacgggcaa B | 11045 | |
| 694 | AGUGCCAUUU G UUCAG | 2461 | 20082 | HBV-694 GCl.Rz-5/10 stab2 | cₛuₛgₛa uGAUₛg gcauGcacuaugc gcg aaauggcacu B | 11046 | |
| 1265 | CUCCUCUGCC G AUCCA | 2462 | 20083 | HBV-1265 GCl.Rz-5/10 stab2 | uₛgₛgₛu uGAUₛg gcauGcacuaugc gcg ggcagaggag B | 11047 | |
| 1601 | CUUCACCUCU G CACGU | 2463 | 20084 | HBV-1601 GCl.Rz-5/10 stab2 | aₛcₛgₛg uGAUₛg gcauGcacuaugc gcg agaggugaag B | 11048 | |
| 1881 | CCUCCAAGCU G UGCCU | 2464 | 20085 | HBV-1881 GCl.Rz-5/10 stab2 | aₛgₛgₛa uGAUₛg gcauGcacuaugc gcg agcuuggagg B | 11049 | |
| 1883 | UCCAAGCUGU G CCUUG | 2465 | 20086 | HBV-1883 GCl.Rz-5/10 stab2 | cₛaₛaₛg uGAUₛg gcauGcacuaugc gcg acagcuugga B | 11050 | |
| 2388 | GAACUCCCUC G CCUCG | 2466 | 20087 | HBV-2388 GCl.Rz-5/10 stab2 | cₛgₛaₛg uGAUₛg gcauGcacuaugc gcg gagggaguuc B | 11051 | |
| 381 | GCUGGAU G UGUCUGC | 2467 | 20091 | HBV-381 Zin.Rz-7 amino stab2 | gₛcₛaₛgₛaca GccgaaagGCGaGugaGGuCu auccagc B | | |
| 392 | CUGCGGC G UUUUAUC | 2468 | 20092 | HBV-392 Zin.Rz-7 amino stab2 | gₛaₛuₛaₛaaa GccgaaagGCGaGugaGGuCu gccgcag B | | |
| 420 | UCCUGCU G CUAUGCC | 2469 | 20093 | HBV-420 Zin.Rz-7 amino stab2 | gₛgₛcₛaₛuag GccgaaagGCGaGugaGGuCu agcagga B | | |
| 648 | UAUGGGA G UGGGCCU | 2470 | 20094 | HBV-648 Zin.Rz-7 amino stab2 | aₛgₛgₛcₛcca GccgaaagGCGaGugaGGuCu ucccaua B | | |
| 711 | UCGUAGG G CUUUCCC | 2471 | 20095 | HBV-711 Zin.Rz-7 amino stab2 | gₛgₛgₛaₛaag GccgaaagGCGaGugaGGuCu ccuacga B | | |
| 1262 | CUCCUCU G CCGAUCC | 2472 | 20096 | HBV-1262 Zin.Rz-7 amino stab2 | gₛgₛaₛuₛcgg GccgaaagGCGaGugaGGuCu agaggag B | | |
| 1835 | CACCUCU G CCUAAUC | 2473 | 20097 | HBV-1835 Zin.Rz-7 amino stab2 | gₛaₛuₛuₛagg GccgaaagGCGaGugaGGuCu agaggug B | | |
| 2388 | CUCCCUC G CCUCGCA | 2474 | 20098 | HBV-2388 Zin.Rz-7 amino stab2 | uₛgₛcₛgₛagg GccgaaagGCGaGugaGGuCu gagggag B | | |
| 192 | GACCCCU G CUCGUGU | 2475 | 20099 | HBV-192 Zin.Rz-7 amino stab2 | aₛcₛaₛcₛgag GccgaaagGCGaGugaGGuCu agggguc B | | |
| 198 | UGCUCGU C UUACAGG | 2476 | 20100 | HBV-198 Zin.Rz-7 amino stab2 | cₛcₛuₛgₛuaa GccgaaagGCGaGugaGGuCu acgagca B | | |
| 315 | AAAAUUC G CAGUCCC | 2477 | 20101 | HBV-315 Zin.Rz-7 amino stab2 | gₛgₛgₛaₛcug GccgaaagGCGaGugaGGuCu gaauuuu B | | |
| 383 | GGAUGU G UCUGCG | 2478 | 20102 | HBV-383 Zin.Rz-6 amino stab2 | cₛgₛcₛaₛga GccgaaagGCGaGugaGGuCu acaucc B | | |
| 383 | UGGAUGU G UCUGCGG | 2479 | 20103 | HBV-383 Zin.Rz-7 amino stab2 | cₛcₛgₛcₛaga GccgaaagGCGaGugaGGuCu acaucca B | | |
| 387 | GUGUCU G CGGCGU | 2480 | 20104 | HBV-387 Zin.Rz-6 amino stab2 | aₛcₛgₛcₛcg GccgaaagGCGaGugaGGuCu agacac B | | |
| 390 | GUCUGCG G CGUUUUA | 2481 | 20105 | HBV-390 Zin.Rz-7 amino stab2 | uₛaₛaₛaₛacg GccgaaagGCGaGugaGGuCu cgcagac B | | |
| 392 | UGCGGC G UUUUAU | 2482 | 20106 | HBV-392 Zin.Rz-6 amino stab2 | aₛuₛaₛaₛaa GccgaaagGCGaGugaGGuCu gccgca B | | |
| 425 | UGCUAU G CCUCAU | 2483 | 20107 | HBV-425 Zin.Rz-6 amino stab2 | aₛuₛgₛaₛgg GccgaaagGCGaGugaGGuCu auagca B | | |
| 425 | CUGCUAU G CCUCAUC | 2484 | 20108 | HBV-425 Zin.Rz-7 amino stab2 | gₛaₛuₛgₛagg GccgaaagGCGaGugaGGuCu auagcag B | | |
| 468 | GUAUGUU G CCCGUUU | 2485 | 20109 | HBV-468 Zin.Rz-7 amino stab2 | aₛaₛaₛcₛggg GccgaaagGCGaGugaGGuCu aacauac B | | |
| 476 | CCCGUUU G UCCUCUA | 2486 | 20110 | HBV-476 Zin.Rz-7 amino stab2 | uₛaₛgₛaₛgga GccgaaagGCGaGugaGGuCu aaacggg B | | |
| 648 | AUGGGA G UGGGCC | 2487 | 20111 | HBV-648 Zin.Rz-6 amino stab2 | gₛgₛcₛcₛca GccgaaagGCGaGugaGGuCu ucccau B | | |
| 694 | GCCAUUU G UUCAGUG | 2488 | 20112 | HBV-694 Zin.Rz-7 amino stab2 | cₛaₛcₛuₛgaa GccgaaagGCGaGugaGGuCu aaauggc B | | |
| 699 | UUGUUCA G UGGUUCG | 2489 | 20113 | HBV-699 Zin.Rz-7 amino stab2 | cₛgₛaₛaₛcca GccgaaagGCGaGugaGGuCu ugaacaa B | | |
| 1262 | UCCUCU G CCGAUC | 2490 | 20114 | HBV-1262 Zin.Rz-6 amino stab2 | gₛaₛuₛcₛgg GccgaaagGCGaGugaGGuCu agagga B | | |
| 1440 | CCCGUCG G CGCUGAA | 2491 | 20115 | HBV-1440 Zin.Rz-7 amino stab2 | uₛuₛcₛaₛgcg GccgaaagGCGaGugaGGuCu cgacggg B | | |
| 1526 | CACGGG G CGCACC | 2492 | 20116 | HBV-1526 Zin.Rz-6 amino stab2 | gₛgₛuₛgₛcg GccgaaagGCGaGugaGGuCu cccgug B | | |
| 1526 | CCACGGG G CGCACCU | 2493 | 20117 | HBV-1526 Zin.Rz-7 amino stab2 | aₛgₛgₛuₛgcg GccgaaagGCGaGugaGGuCu cccgugg B | | |
| 1557 | CCCGUCU G UGCCUUC | 2494 | 20118 | HBV-1557 Zin.Rz-7 amino stab2 | gₛaₛaₛgₛgca GccgaaagGCGaGugaGGuCu agacggg B | | |
| 1559 | CGUCUGU G CCUUCUC | 2495 | 20119 | HBV-1559 Zin.Rz-7 amino stab2 | gₛaₛgₛaₛagg GccgaaagGCGaGugaGGuCu acagacg B | | |
| 1590 | GCACUUC G CUUCACC | 2496 | 20120 | HBV-1590 Zin.Rz-7 amino stab2 | gₛgₛuₛgₛaag GccgaaagGCGaGugaGGuCu gaagugc B | | |
| 1835 | ACCUCU G CCUAAU | 2497 | 20121 | HBV-1835 Zin.Rz-6 amino stab2 | aₛuₛuₛaₛgg GccgaaagGCGaGugaGGuCu agaggu B | | |
| 2311 | ACCAAAU G CCCCUAU | 2498 | 20122 | HBV-2311 Zin.Rz-7 amino stab2 | aₛuₛaₛgₛggg GccgaaagGCGaGugaGGuCu auuuggu B | | |
| 2420 | CCGCGUC G CAGAAGA | 2499 | 20123 | HBV-2420 Zin.Rz-7 amino stab2 | uₛcₛuₛuₛcug GccgaaagGCGaGugaGGuCu gacgcgg B | | |
| 65 | CCUGCUG G UGGCUCC | 2500 | 20124 | HBV-65 Zin.Rz-7 amino stab2 | gₛgₛaₛgₛcca GccgaaagGCGaGugaGGuCu cagcagg B | | |
| 192 | ACCCCU G CUCGUG | 2501 | 20125 | HBV-192 Zin.Rz-6 amino stab2 | cₛaₛcₛgₛag GccgaaagGCGaGugaGGuCu aggggu B | | |
| 198 | GCUCGU G UUACAG | 2502 | 20126 | HBV-198 Zin.Rz-6 amino stab2 | cₛuₛgₛuₛaa GccgaaagGCGaGugaGGuCu acgagc B | | |
| 258 | UAGACUC G UGGUGGA | 2503 | 20127 | HBV-258 Zin.Rz-7 amino stab2 | uₛcₛcₛaₛcca GccgaaagGCGaGugaGGuCu gagucua B | | |
| 261 | ACUCGUG G UGGACUU | 2504 | 20128 | HBV-261 Zin.Rz-7 amino stab2 | aₛaₛgₛuₛcca GccgaaagGCGaGugaGGuCu cacgagu B | | |
| 315 | AAAUUC G CAGUCC | 2505 | 20129 | HBV-315 Zin.Rz-6 amino stab2 | gₛgₛaₛcₛug GccgaaagGCGaGugaGGuCu gaauuu B | | |
| 381 | CUGGAU G UGUCUG | 2506 | 20130 | HBV-381 Zin.Rz-6 amino stab2 | cₛaₛgₛaₛca GccgaaagGCGaGugaGGuCu auccag B | | |
| 387 | UGUGUCU G CGGCGUU | 2507 | 20131 | HBV-387 Zin.Rz-7 amino stab2 | aₛaₛcₛgₛccg GccgaaagGCGaGugaGGuCu agacaca B | | |
| 390 | UCUGCG G CGUUUU | 2508 | 20132 | HBV-390 Zin.Rz-6 amino stab2 | aₛaₛaₛaₛcg GccgaaagGCGaGugaGGuCu cgcaga B | | |
| 417 | CAUCCU G CUGCUA | 2509 | 20133 | HBV-417 Zin.Rz-6 amino stab2 | uₛaₛgₛcₛag GccgaaagGCGaGugaGGuCu aggaug B | | |
| 420 | CCUGCU G CUAUGC | 2510 | 20134 | HBV-420 Zin.Rz-6 amino stab2 | gₛcₛaₛuₛag GccgaaagGCGaGugaGGuCu agcagg B | | |
| 468 | UAUGUU G CCCGUU | 2511 | 20135 | HBV-468 Zin.Rz-6 amino stab2 | aₛaₛcₛgₛgg GccgaaagGCGaGugaGGuCu aacaua B | | |
| 476 | CCGUUU G UCCUCU | 2512 | 20136 | HBV-476 Zin.Rz-6 amino stab2 | aₛgₛaₛgₛga GccgaaagGCGaGugaGGuCu aaacgg B | | |
| 677 | GGCUCA G UUUACU | 2513 | 20137 | HBV-677 Zin.Rz-6 amino stab2 | aₛgₛuₛaₛaa GccgaaagGCGaGugaGGuCu ugagcc B | | |
| 677 | UGGCUCA G UUUACUA | 2514 | 20138 | HBV-677 Zin.Rz-7 amino stab2 | uₛaₛgₛuₛaaa GccgaaagGCGaGugaGGuCu ugagcca B | | |
| 685 | UUACUA G UGCCAU | 2515 | 20139 | HBV-685 Zin.Rz-6 amino stab2 | aₛuₛgₛgₛca GccgaaagGCGaGugaGGuCu uaguaa B | | |
| 685 | UUUACUA G UGCCAUU | 2516 | 20140 | HBV-685 Zin.Rz-7 amino stab2 | aₛaₛuₛgₛgca GccgaaagGCGaGugaGGuCu uaguaaa B | | |
| 687 | UACUAGU G CCAUUUG | 2517 | 20141 | HBV-687 Zin.Rz-7 amino stab2 | cₛaₛaₛaₛugg GccgaaagGCGaGugaGGuCu acuagua B | | |
| 699 | UGUUCA G UGGUUC | 2518 | 20142 | HBV-699 Zin.Rz-6 amino stab2 | gₛaₛaₛcₛca GccgaaagGCGaGugaGGuCu ugaaca B | | |
| 702 | UCAGUG G UUCGUA | 2519 | 20143 | HBV-702 Zin.Rz-6 amino stab2 | uₛaₛcₛgₛaa GccgaaagGCGaGugaGGuCu cacuga B | | |
| 702 | UUCAGUG G UUCGUAG | 2520 | 20144 | HBV-702 Zin.Rz-7 amino stab2 | cₛuₛaₛcₛgaa GccgaaagGCGaGugaGGuCu cacugaa B | | |
| 711 | CGUAGG G CUUUCC | 2521 | 20145 | HBV-711 Zin.Rz-6 amino stab2 | gₛgₛaₛaₛag GccgaaagGCGaGugaGGuCu ccuacg B | | |
| 1006 | UUGUGG G UCUUUU | 2522 | 20146 | HBV-1006 Zin.Rz-6 amino stab2 | aₛaₛaₛaₛga GccgaaagGCGaGugaGGuCu ccacaa B | | |
| 1103 | UUUCUC G CCAACU | 2523 | 20147 | HBV-1103 Zin.Rz-6 amino stab2 | aₛgₛuₛuₛgg GccgaaagGCGaGugaGGuCu gagaaa B | | |
| 1103 | CUUUCUC G CCAACUU | 2524 | 20148 | HBV-1103 Zin.Rz-7 amino stab2 | aₛaₛgₛuₛugg GccgaaagGCGaGugaGGuCu gagaaag B | | |
| 1184 | GCCAAGU G UUUGCUG | 2525 | 20149 | HBV-1184 Zin.Rz-7 amino stab2 | cₛaₛgₛcₛaaa GccgaaagGCGaGugaGGuCu acuuggc B | | |
| 1440 | CCGUCG G CGCUGA | 2526 | 20150 | HBV-1440 Zin.Rz-6 amino stab2 | uₛcₛaₛgₛcg GccgaaagGCGaGugaGGuCu cgacgg B | | |
| 1442 | GUCGGC G CUGAAU | 2527 | 20151 | HBV-1442 Zin.Rz-6 amino stab2 | aₛuₛuₛcₛag GccgaaagGCGaGugaGGuCu gccgac B | | |
| 1442 | CGUCGGC G CUGAAUC | 2528 | 20152 | HBV-1442 Zin.Rz-7 amino stab2 | gₛaₛuₛuₛcag GccgaaagGCGaGugaGGuCu gccgacg B | | |
| 1553 | CUCCCC G UCUGUG | 2529 | 20153 | HBV-1553 Zin.Rz-6 amino stab2 | cₛaₛcₛaₛga GccgaaagGCGaGugaGGuCu ggggag B | | |
| 1557 | CCGUCU G UGCCUU | 2530 | 20154 | HBV-1557 Zin.Rz-6 amino stab2 | aₛaₛgₛgₛca GccgaaagGCGaGugaGGuCu agacgg B | | |
| 1559 | GUCUGU G CCUUCU | 2531 | 20155 | HBV-1559 Zin.Rz-6 amino stab2 | aₛgₛaₛaₛgg GccgaaagGCGaGugaGGuCu acagac B | | |
| 1583 | CCGUGU G CACUUC | 2532 | 20156 | HBV-1583 Zin.Rz-6 amino stab2 | gₛaₛaₛgₛug GccgaaagGCGaGugaGGuCu acacgg B | | |
| 1590 | CACUUC G CUUCAC | 2533 | 20157 | HBV-1590 Zin.Rz-6 amino stab2 | gₛuₛgₛaₛag GccgaaagGCGaGugaGGuCu gaagug B | | |
| 1622 | ACCACC G UGAACG | 2534 | 20158 | HBV-1622 Zin.Rz-6 amino stab2 | cₛgₛuₛuₛca GccgaaagGCGaGugaGGuCu gguggu B | | |
| 1870 | UGUUCAA G CCUCCAA | 2535 | 20159 | HBV-1870 Zin.Rz-7 amino stab2 | uₛuₛgₛgₛagg GccgaaagGCGaGugaGGuCu uugaaca B | | |
| 1881 | CCAAGCU G UGCCUUG | 2536 | 20160 | HBV-1881 Zin.Rz-7 amino stab2 | cₛaₛaₛgₛgca GccgaaagGCGaGugaGGuCu agcuugg B | | |
| 1883 | AGCUGU G CCUUGG | 2537 | 20161 | HBV-1883 Zin.Rz-6 amino stab2 | cₛcₛaₛaₛgg GccgaaagGCGaGugaGGuCu acagcu B | | |
| 1883 | AAGCUGU G CCUUGGG | 2538 | 20162 | HBV-1883 Zin.Rz-7 amino stab2 | cₛcₛcₛaₛagg GccgaaagGCGaGugaGGuCu acagcuu B | | |
| 2311 | CCAAAU G CCCCUA | 2539 | 20163 | HBV-2311 Zin.Rz-6 amino stab2 | uₛaₛgₛgₛgg GccgaaagGCGaGugaGGuCu auuugg B | | |
| 2347 | ACUGUU G UUAGAC | 2540 | 20164 | HBV-2347 Zin.Rz-6 amino stab2 | gₛuₛcₛuₛaa GccgaaagGCGaGugaGGuCu aacagu B | | |
| 2364 | AGGCAG G UCCCCU | 2541 | 20165 | HBV-2364 Zin.Rz-6 amino stab2 | aₛgₛgₛgₛga GccgaaagGCGaGugaGGuCu cugccu B | | |
| 2364 | GAGGCAG G UCCCCUA | 2542 | 20166 | HBV-2364 Zin.Rz-7 amino stab2 | uₛaₛgₛgₛgga GccgaaagGCGaGugaGGuCu cugccuc B | | |
| 2388 | UCCCUC G CCUCGC | 2543 | 20167 | HBV-2388 Zin.Rz-6 amino stab2 | gₛcₛgₛaₛgg GccgaaagGCGaGugaGGuCu gaggga B | | |
| 2393 | CGCCUC G CAGACG | 2544 | 20168 | HBV-2393 Zin.Rz-6 amino stab2 | cₛgₛuₛcₛug GccgaaagGCGaGugaGGuCu gaggcg B | | |
| 2417 | CGCCGC G UCGCAG | 2545 | 20169 | HBV-2417 Zin.Rz-6 amino stab2 | cₛuₛgₛcₛga GccgaaagGCGaGugaGGuCu gcggcg B | | |
| 2420 | CGCGUC G CAGAAG | 2546 | 20170 | HBV-2420 Zin.Rz-6 amino stab2 | cₛuₛuₛcₛug GccgaaagGCGaGugaGGuCu gacgcg B | | |
| 2474 | CAUAAG G UGGGAA | 2547 | 20171 | HBV-2474 Zin.Rz-6 amino stab2 | uₛuₛcₛcₛca GccgaaagGCGaGugaGGuCu cuuaug B | | |
| 381 | GCUGGAU G UGUCUGC | 2467 | 20172 | HBV-381 Amb.Rz-7 stab2 | gₛcₛaₛgₛaca gga L ucCCUUCaagga L ucCGGG auccagc B | 11133 | |
| 648 | UAUGGGA G UGGGCCU | 2470 | 20173 | HBV-648 Amb.Rz-7 stab2 | aₛgₛgₛcₛcca gga L ucCCUUCaagga L ucCGGG ucccaua B | 11134 | |
| 198 | UGCUCGU G UUACAGG | 2476 | 20174 | HBV-198 Amb.Rz-7 stab2 | cₛcₛuₛgₛuaa gga L ucCCUUCaagga L ucCGGG acgagca B | 11135 | |
| 377 | UAUCGCU G GAUGUGU | 2548 | 20175 | HBV-377 Amb.Rz-7 stab2 | aₛcₛaₛcₛauc gga L ucCCUUCaagga L ucCGGG agcgaua B | 11136 | |
| 378 | AUCGCUG G AUGUGUC | 2549 | 20176 | HBV-378 Amb.Rz-7 stab2 | gₛaₛcₛaₛcau gga L ucCCUUCaagga L ucCGGG cagcgau B | 11137 | |
| 383 | UGGAUGU G UCUGCGG | 2479 | 20177 | HBV-383 Amb.Rz-7 stab2 | cₛcₛgₛcₛaga gga L ucCCUUCaagga L ucCGGG acaucca B | 11138 | |
| 383 | GGAUGU G UCUGCG | 2478 | 20178 | HBV-383 Amb.Rz-6 stab2 | cₛgₛcₛaₛga gga L ucCCUUCaagga L ucCGGG acaucc B | 11139 | |
| 648 | AUGGGA G UGGGCC | 2487 | 20179 | HBV-648 Amb.Rz-6 stab2 | gₛgₛcₛcₛca gga L ucCCUUCaagga L ucCGGG ucccau B | 11140 | |
| 650 | UGGGAGU G GGCCUCA | 2550 | 20180 | HBV-650 Amb.Rz-7 stab2 | uₛgₛaₛgₛgcc gga L ucCCUUCaagga L ucCGGG acuccca B | 11141 | |
| 650 | GGGAGU G GGCCUC | 2551 | 20181 | HBV-650 Amb.Rz-6 stab2 | gₛaₛgₛgₛcc gga L ucCCUUCaagga L ucCGGG acuccc B | 11142 | |
| 694 | GCCAUUU G UUCAGUG | 2488 | 20182 | HBV-694 Amb.Rz-7 stab2 | cₛaₛcₛuₛgaa gga L ucCCUUCaagga L ucCGGG aaauggc B | 11143 | |
| 699 | UUGUUCA G UGGUUCG | 2489 | 20183 | HBV-699 Amb.Rz-7 stab2 | cₛgₛaₛaₛcca gga L ucCCUUCaagga L ucCGGG ugaacaa B | 11144 | |
| 701 | GUUCAGU G GUUCGUA | 2552 | 20184 | HBV-701 Amb.Rz-7 stab2 | uₛaₛcₛgₛaac gga L ucCCUUCaagga L ucCGGG acugaac B | 11145 | |
| 710 | UUCGUAG G GCUUUCC | 2553 | 20185 | HBV-710 Amb.Rz-7 stab2 | gₛgₛaₛaₛagc gga L ucCCUUCaagga L ucCGGG cuacgaa B | 11146 | |
| 1525 | CCACGG G GCGCAC | 2554 | 20186 | HBV-1525 Amb.Rz-6 stab2 | gₛuₛgₛcₛgc gga L ucCCUUCaagga L ucCGGG ccgugg B | 11147 | |
| 1624 | CACCGU G AACGCC | 2555 | 20187 | HBV-1624 Amb.Rz-6 stab2 | gₛgₛcₛgₛuu gga L ucCCUUCaagga L ucCGGG acggug B | 11148 | |
| 2069 | CACUCA G GCAAGC | 2556 | 20188 | HBV-2069 Amb.Rz-6 stab2 | gₛcₛuₛuₛgc gga L ucCCUUCaagga L ucCGGG ugagug B | 11149 | |
| 2375 | CCUAGAA G AAGAACU | 2557 | 20189 | HBV-2375 Amb.Rz-7 stab2 | aₛgₛuₛuₛcuu gga L ucCCUUCaagga L ucCGGG uucuagg B | 11150 | |
| 2476 | AUAAGGU G GGAAACU | 2558 | 20190 | HBV-2476 Amb.Rz-7 stab2 | aₛgₛuₛuₛucc gga L ucCCUUCaagga L ucCGGG accuuau B | 11151 | |
| 65 | CCUGCUG G UGGCUCC | 2500 | 20191 | HBV-65 Amb.Rz-7 stab2 | gₛgₛaₛgₛcca gga L ucCCUUCaagga L ucCGGG cagcagg B | 11152 | |
| 67 | GCUGGU G GCUCCA | 2559 | 20192 | HBV-67 Amb.Rz-6 stab2 | uₛgₛgₛaₛgc gga L ucCCUUCaagga L ucCGGG accagc B 11153 | | |
| 198 | GCUCGU G UUACAG | 2502 | 20193 | HBV-198 Amb.Rz-6 stab2 | cₛuₛgₛuₛaa gga L ucCCUUCaagga L ucCGGG acgagc B | 11154 | |
| 260 | GACUCGU G GUGGACU | 2560 | 20194 | HBV-260 Amb.Rz-7 stab2 | aₛgₛuₛcₛcac gga L ucCCUUCaagga L ucCGGG acgaguc B | 11155 | |
| 263 | UCGUGGU G GACUUCU | 2561 | 20195 | HBV-263 Amb.Rz-7 stab2 | aₛgₛaₛaₛguc gga L ucCCUUCaagga L ucCGGG accacga B | 11156 | |
| 377 | AUCGCU G GAUGUG | 2562 | 20196 | HBV-377 Amb.Rz-6 stab2 | cₛaₛcₛaₛuc gga L ucCCUUCaagga L ucCGGG agcgau B | 11157 | |
| 378 | UCGCUG G AUGUGU | 2563 | 20197 | HBV-378 Amb.Rz-6 stab2 | aₛcₛaₛcₛau gga L ucCCUUCaagga L ucCGGG cagcga B | 11158 | |
| 476 | CCGUUU G UCCUCU | 2512 | 20198 | HBV-476 Amb.Rz-6 stab2 | aₛgₛaₛgₛga gga L ucCCUUCaagga L ucCGGG aaacgg B | 11159 | |
| 651 | GGGAGUG G GCCUCAG | 2564 | 20199 | HBV-651 Amb.Rz-7 stab2 | cₛuₛgₛaₛggc gga L ucCCUUCaagga L ucCGGG cacuccc B | 11160 | |
| 677 | UGGCUCA G UUUACUA | 2514 | 20200 | HBV-677 Amb.Rz-7 stab2 | uₛaₛgₛuₛaaa gga L ucCCUUCaagga L ucCGGG ugagcca B | 11161 | |
| 685 | UUUACUA G UGCCAUU | 2516 | 20201 | HBV-685 Amb.Rz-7 stab2 | aₛaₛuₛgₛgca gga L ucCCUUCaagga L ucCGGG uaguaaa B | 11162 | |
| 702 | UUCAGUG G UUCGUAG | 2520 | 20202 | HBV-702 Amb.Rz-7 stab2 | cₛuₛaₛcₛgaa gga L ucCCUUCaagga L ucCGGG cacugaa B | 11163 | |
| 709 | GUUCGUA G GGCUUUC | 2565 | 20203 | HBV-709 Amb.Rz-7 stab2 | gₛaₛaₛaₛgcc gga L ucCCUUCaagga L ucCGGG uacgaac B | 11164 | |
| 710 | UCGUAG G GCUUUC | 2566 | 20204 | HBV-710 Amb.Rz-6 stab2 | gₛaₛaₛaₛgc gga L ucCCUUCaagga L ucCGGG cuacga B | 11165 | |
| 747 | UAUGGAU G AUGUGGU | 2567 | 20205 | HBV-747 Amb.Rz-7 stab2 | aₛcₛcₛaₛcau gga L ucCCUUCaagga L ucCGGG auccaua B | 11166 | |
| 1557 | CCGUCU G UGCCUU | 2530 | 20206 | HBV-1557 Amb.Rz-6 stab2 | aₛaₛgₛgₛca gga L ucCCUUCaagga L ucCGGG agacgg B | 11167 | |
| 1881 | CCAAGCU G UGCCUUG | 2536 | 20207 | HBV-1881 Amb.Rz-7 stab2 | cₛaₛaₛgₛgca gga L ucCCUUCaagga L ucCGGG agcuugg B | 11168 | |
| 2347 | ACUGUU G UUAGAC | 2540 | 20208 | HBV-2347 Amb.Rz-6 stab2 | gₛuₛcₛuₛaa gga L ucCCUUCaagga L ucCGGG aacagu B | 11169 | |
| 2375 | CUAGAA G AAGAAC | 2568 | 20209 | HBV-2375 Amb.Rz-6 stab2 | gₛuₛuₛcₛuu gga L ucCCUUCaagga L ucCGGG uucuag B | 11170 | |
| 2378 | GAAGAA G AACUCC | 2569 | 20210 | HBV-2378 Amb.Rz-6 stab2 | gₛgₛaₛgₛuu gga L ucCCUUCaagga L ucCGGG uucuuc B | 11171 | |
| 2423 | CGUCGCA G AAGAUCU | 2570 | 20211 | HBV-2423 Amb.Rz-7 stab2 | aₛgₛaₛuₛcuu gga L ucCCUUCaagga L ucCGGG ugcgacg B | 11172 | |
| 2426 | GCAGAA G AUCUCA | 2571 | 20212 | HBV-2426 Amb.Rz-6 stab2 | uₛgₛaₛgₛau gga L ucCCUUCaagga L ucCGGG uucugc B | 11173 | |
| 2426 | CGCAGAA G AUCUCAA | 2572 | 20213 | HBV-2426 Amb.Rz-7 stab2 | uₛuₛgₛaₛgau gga L ucCCUUCaagga L ucCGGG uucugcg B | 11174 | |
| 2476 | UAAGGU G GGAAAC | 2573 | 20214 | HBV-2476 Amb.Rz-6 stab2 | gₛuₛuₛuₛcc gga L ucCCUUCaagga L ucCGGG accuua B | 11175 | |
| 2477 | UAAGGUG G GAAACUU | 2574 | 20215 | HBV-2477 Amb.Rz-7 stab2 | aₛaₛgₛuₛuuc gga L ucCCUUCaagga L ucCGGG caccuua B | 11176 | |
| 2477 | AAGGUG G GAAACU | 2575 | 20216 | HBV-2477 Amb.Rz-6 stab2 | aₛgₛuₛuₛuc gga L ucCCUUCaagga L ucCGGG caccuu B | 11177 | |
| 1607 | UGCACGU C GCAUGGA | 2576 | 20697 | HBV-1607 Rz-7 allyl stab1 (7/4) | uₛcₛcₛaₛugc cUGAuGaggccguuaggccGaa Acgugca B | | |
| 1887 | GUGCCU U GGGUGG | 2577 | 20698 | HBV-1887 Rz-6 allyl stab1 (6/4) | cₛcₛaₛcₛcc cUGAuGaggccguuaggccGaa Aggcac B | | |
| 1607 | GCACGU C GCAUGG | 2374 | 20699 | HBV-1607 Rz-6 allyl stab1 (6/3) | cₛcₛaₛuₛgc cUGAuGaggcguuagccGaa Acgugc B | | |
| 1607 | UGCACGU C GCAUGGA | 2576 | 20700 | HBV-1607 Rz-7 allyl stab1 (7/3) | uₛcₛcₛaₛugc cUGAuGaggcguuagccGaa Acgugca B | | |
| 1887 | GUGCCU U GGGUGG | 2577 | 20701 | HBV-1887 Rz-6 allyl stab1 (6/3) | cₛcₛaₛcₛcc cUGAuGaggcguuagccGaa Aggcac B | | |
| 1887 | UGUGCCU U GGGUGGC | 2420 | 20702 | HBV-1887 Rz-7 allyl stab1 (7/3) | gₛcₛcₛaₛccc cUGAuGaggcguuagccGaa Aggcaca B | | |
| 313 | CCAAAAU U CGCAGUC | 2346 | 22798 | HBV-313 Rz-7 Ome stab1 | gacugcg CUGAUGAggccguuaggccGAA Auuuugg B | 11184 | |
| 408 | UCUUCCU C UGCAUCC | 2349 | 22799 | HBV-408 Rz-7 Ome stab1 | ggaugca CUGAUGAggccguuaggccGAA Aggaaga B | 11185 | |
| 1756 | AGGAGGU U AGGUUAA | 2353 | 22800 | HBV-1756 Rz-7 Ome stab1 | uuaaccu CUGAUGAggccguuaggccGAA Accuccu B | 11186 | |
| 10 | CUCCACC A CUUUCCA | 2356 | 22770 | HBV-10 CHz-7 Ome stab1 | uggaaag CUGAUGAggccguuaggccGAA Iguggag B | 11187 | |
| 335 | UCCAGUC A CUCACCA | 2357 | 22771 | HBV-335 CHz-7 Ome stab1 | uggugag CUGAUGAggccguuaggccGAA Iacugga B | 11188 | |
| 273 | CUUCUCU C AAUUUUC | 2399 | | 22645 | HBV-273 Rz-7 allyl stab1 (7/3-GUUA) | gₛaₛaₛaₛauu cUGAuGagccguuaggcGaa Agagaag B | 11189 |
| 273 | CUUCUCU C AAUUUUC | 2399 | 22646 | HBV-273 Rz-7 allyl stab1 (7/4-GUUA) | gₛaₛaₛaₛauu cUGAuGaggccguuaggccGaa Agagaag B | 11190 | |
| 273 | CUUCUCU C AAUUUUC | 2399 | 22648 | HBV-273 Rz-7 allyl stab1 (7/3-GAAA) | gₛaₛaₛaₛauu cUGAuGagccgaaaggcGaa Agagaag B | 11191 | |
| 273 | CUUCUCU C AAUUUUC | 2578 | 22650 | HBV-273 Rz-7 allyl stab1 (7/4-GAAA) | gₛaₛaₛaₛauu cUGAuGaggccgaaaggccGaa Agagaag B | 11192 | |
| 273 | UUCUCU C AAUUUU | | 22644 | HBV-273 Rz-6 allyl stab1 (6/3-GUUA) | aₛaₛaₛaₛuu cUGAuGagccguuaggcGaa Agagaa B | 11193 | |
| 273 | UUCUCU C AAUUUU | | 22647 | HBV-273 Rz-6 allyl stab1 (6/3-GAAA) | aₛaₛaₛaₛuu cUGAuGagccgaaaggcGaa Agagaa B | 11194 | |
| 273 | UUCUCU C AAUUUU | 2579 | 22649 | HBV-273 Rz-6 allyl stab1 (6/4-GAAA) | aₛaₛaₛaₛuu cUGAuGaggccgaaaggccGaa Agagaa B | 11195 | |
| 350 | ACCUGUU G UCCUCCA | 2580 | 22714 | HBV-350 GCl.Rz-7 5ribo stab3 | uggagga uGAUg gcauGcacuaugc gCg aacaggu B | 11196 | |
| 1253 | CCUUUGU G UCUCCUC | 2581 | 22715 | HBV-1253 GCl.Rz-7 5ribo stab3 | gaggaga uGAUg gcauGcacuaugc gCg acaaagg B | 11197 | |
| 1856 | UGUUCAU G UCCUACU | 2582 | 22716 | HBV-1856 GCl.Rz-7 5ribo stab3 | aguagga uGAUg gcauGcacuaugc gCg augaaca B | 11198 | |
| 1966 | GCCUUCU G ACUUCUU | 2583 | 22717 | HBV-1966 GCl.Rz-7 5ribo stab3 | aagaagu uGAUg gcauGcacuaugc gCg agaaggc B | 11199 | |
| 3132 | UCCUCCU G CCUCCAC | 2584 | 22718 | HBV-3132 GCl.Rz-7 5ribo stab3 | guggagg uGAUg gcauGcacuaugc gCg aggagga B | 11200 | |
| 332 | AUCUCCA G UCACUCA | 2579 | 22742 | HBV-332 Zin.Rz-7 amino stab4 | ugaguga gccgaaaggCgagugaGGuCu uggagau B | 11201 | |
| 350 | ACCUGUU G UCCUCCA | 2585 | 22743 | HBV-350 Zin.Rz-7 amino stab4 | uggagga gccgaaaggCgagugaGGuCu aacaggu B | 11202 | |
| 410 | UUCCUCU G CAUCCUG | 2580 | 22744 | HBV-410 Zin.Rz-7 amino stab4 | caggaug gccgaaaggCgagugaGGuCu agaggaa B | 11203 | |
| 1253 | CCUUUGU G UCUCCUC | 2586 | 22745 | HBV-1253 Zin.Rz-7 amino stab4 | gaggaga gccgaaaggCgagugaGGuCu acaaagg B | 11204 | |
| 1754 | GGAGGAG G UUAGGUU | 2587 | 22746 | HBV-1754 Zin.Rz-7 amino stab4 | aaccuaa gccgaaaggCgagugaGGuCu cuccucc B | 11205 | |
| 407 | AUCUUCC U CUGCAUC | 2588 | 22772 | HBV-407 CHz-7 Ome stab1 | gaugcag CUGAUGAggccguuaggccGAA Igaagau B | 11206 | |
| 1848 | UCAUCUC A UGUUCAU | 2589 | 22773 | HBV-1848 CHz-7 Ome stab1 | augaaca CUGAUGAggccguuaggccGAA Iagauga B | 11207 | |
| 3124 | GCAGCUC C UCCUCCU | 2590 | 22774 | HBV-3124 CHz-7 Ome stab1 | aggagga CUGAUGAggccguuaggccGAA Iagcugc B | 11208 | |
| 2165 | GUCAGCU A UGUCAAC | 2591 | 22801 | HBV-2165 Rz-7 Ome stab1 | guugaca CUGAUGAggccguuaggccGAA Agcugac B | 11209 | |
| 2706 | CCGUAUU A UCCAGAG | 2579 | 22802 | HBV-2706 Rz-7 Ome stab1 | cucugga CUGAUGAggccguuaggccGAA Aauacgg B | 11210 | |
| 350 | ACCUGUU G UCCUCCA | 2584 | 22966 | HBV-350 Dz-7 stab3 | uggagga GGCTAGCTACAACGA aacaggu B | 11211 | |
| 332 | AUCUCCA G UCACUCA | 2592 | 22967 | HBV-332 Dz-7 stab3 | ugaguga GGCTAGCTACAACGA uggagau B | 11212 | |
| 1840 | CUGCCUA A UCAUCUC | 2593 | 22968 | HBV-1840 Dz-7 stab3 | gagauga GGCTAGCTACAACGA uaggcag B | 11213 | |
| 358 | UCCUCCA A UUUGUCC | 2580 | 22969 | HBV-358 Dz-7 stab3 | ggacaaa GGCTAGCTACAACGA uggagga B | 11214 | |
| 1253 | CCUUUGU G UCUCCUC | 2346 | 22970 | HBV-1253 Dz-7 stab3 | gaggaga GGCTAGCTACAACGA acaaagg B | 11215 | |
| | | | 20599 | SAC | cₛgₛaₛuₛgu cUAGuGacccgaaagggGaa AagaggB | 10834 | |

UPPER CASE = RIBO
UNDERLINE = DEOXY
lower case = 2′-O-methyl
I = inosine
s = phosphorothioate linkage
B= inverted deoxyabasic residue
U= 2′-deoxy-2′-C-allyl Uridine
U = 2′-deoxy-2′-amino Uridine
C = 2′-deoxy-2′-amino Cytidine

TABLE XII
Group Designation and Dosage levels
for HBV transgenic mouse study

| | | | Number | Duration of |
|---|---|---|---|---|
| Group | Compound | Dose | of Mice | Treatment |
| 1 | RPI.18341 | 100 mg/kg/day* | 10 F | 14 days |
| | (site 273) | | | |
| 2 | RPI.18371 | 100 mg/kg/day* | 10 F | 14 days |
| | (site 1833) | | | |
| 3 | RPI.18418 | 100 mg/kg/day* | 10 F | 14 days |
| | (site 1873) | | | |
| 4 | RPI.18372 | 100 mg/kg/day* | 10 F | 14 days |
| | (site 1874) | | | |
| 5 | Saline control | 100 mg/kg/day* | 10 F | 14 days |
| 6 | Untreated | | 10 F | 0 days |

*administered via sc infusion using Alzet ® mini-osmotic pumps

TABLE XIII
GROUP DESIGNATION AND DOSAGE LEVELS FOR HBV
TRANSGENIC MOUSE STUDY

| | | | | Duration |
|---|---|---|---|---|
| | | | Number | of |
| Group | Compound | Dose | of Mice | Treatment |
| 1 | RPI.18341 | 100 mg/kg/day* | 15 (M or F) | 14 days |
| | (site 273) | | | |
| 2 | RPI.18341 | 30 mg/kg/day* | 15 (M or F) | 14 days |
| | (site 273) | | | |
| 3 | RPI.18341 | 10 mg/kg/day* | 15 (M or F) | 14 days |
| | (site 273) | | | |
| 4 | RPI.18371 | 100 mg/kg/day* | 15 (M or F) | 14 days |
| | site 1833 | | | |
| 5 | RPI.18371 | 30 mg/kg/day* | 15 (M or F) | 14 days |
| | site 1833 | | | |
| 6 | RPI.18371 | 10 mg/kg/day* | 15 (M or F) | 14 days |
| | site 1833 | | | |
| 7 | SAC (RPI.20599) | 100 mg/kg/day* | 15 (M or F) | 14 days |
| 8 | SAC (RPI.20599) | 30 mg/kg/day* | 15 (M or F) | 14 days |
| 9 | SAC (RPI.20599) | 10 mg/kg/day* | 15 (M or F) | 14 days |
| 10 | Saline control | 12 μl/day* | 15 (M or F) | 14 days |
| 11 | 3TC ® control | 50 mg/kg/day, | 15 (M or F) | 14 days |
| | | PO | | |

*administered via sc infusion using Alzet ® mini-osmotic pumps

TABLE XIV
HBV RT primer Decoy sequences

| | | |
|---|---|---|
| | | Seq ID |
| Length | Decoy Sequence | No. |

| | | |
|---|---|---|
| 4 | AUUC | 11216 |
| 4 | CAUU | 11217 |
| 4 | UCAU | 11218 |
| 4 | UUCA | 11219 |
| 5 | AUUCA | 11220 |
| 5 | CAUUC | 11221 |
| 5 | UCAUU | 11222 |
| 5 | UUCAU | 11223 |
| 6 | AUUCAU | 11224 |
| 6 | CAUUCA | 11225 |
| 6 | UCAUUC | 11226 |
| 6 | UUCAUU | 11227 |
| 7 | AUUCAUU | 11228 |
| 7 | CAUUCAU | 11229 |
| 7 | UCAUUCA | 11230 |
| 7 | UUCAUUC | 11231 |
| 8 | AUUCAUUC | 11232 |
| 8 | CAUUCAUU | 11233 |
| 8 | UCAUUCAU | 11234 |
| 8 | UUCAUUCA | 11235 |
| 9 | AUUCAUUCA | 11236 |
| 9 | CAUUCAUUC | 11237 |
| 9 | UCAUUCAUU | 11238 |
| 9 | UUCAUUCAU | 11239 |
| 10 | AUUCAUUCAU | 11240 |
| 10 | CAUUCAUUCA | 11241 |
| 10 | UCAUUCAUUC | 11242 |
| 10 | UUCAUUCAUU | 11243 |
| 11 | AUUCAUUCAUU | 11244 |
| 11 | CAUUCAUUCAU | 11245 |
| 11 | UCAUUCAUUCA | 11246 |
| 11 | UUCAUUCAUUC | 11247 |
| 12 | AUUCAUUCAUUC | 11248 |
| 12 | CAUUCAUUCAUU | 11249 |
| 12 | UCAUUCAUUCAU | 11250 |
| 12 | UUCAUUCAUUCA | 11251 |
| 13 | AUUCAUUCAUUCA | 11252 |
| 13 | CAUUCAUUCAUUC | 11253 |
| 13 | UCAUUCAUUCAUU | 11254 |
| 13 | UUCAUUCAUUCAU | 11255 |
| 14 | AUUCAUUCAUUCAU | 11256 |
| 14 | CAUUCAUUCAUUCA | 11257 |
| 14 | UCAUUCAUUCAUUC | 11258 |
| 14 | UUCAUUCAUUCAUU | 11259 |
| 15 | AUUCAUUCAUUCAUU | 11260 |
| 15 | CAUUCAUUCAUUCAU | 11261 |
| 15 | UCAUUCAUUCAUUCA | 11262 |
| 15 | UUCAUUCAUUCAUUC | 11263 |
| 16 | AUUCAUUCAUUCAUUC | 11264 |
| 16 | CAUUCAUUCAUUCAUU | 11265 |
| 16 | UCAUUCAUUCAUUCAU | 11266 |
| 16 | UUCAUUCAUUCAUUCA | 11267 |
| 17 | AUUCAUUCAUUCAUUCA | 11268 |
| 17 | CAUUCAUUCAUUCAUUC | 11269 |
| 17 | UCAUUCAUUCAUUCAUU | 11270 |
| 17 | UUCAUUCAUUCAUUCAU | 11271 |
| 18 | AUUCAUUCAUUCAUUCAU | 11272 |
| 18 | CAUUCAUUCAUUCAUUCA | 11273 |
| 18 | UCAUUCAUUCAUUCAUUC | 11274 |
| 18 | UUCAUUCAUUCAUUCAUU | 11275 |
| 19 | AUUCAUUCAUUCAUUCAUU | 11276 |
| 19 | CAUUCAUUCAUUCAUUCAU | 11277 |
| 19 | UCAUUCAUUCAUUCAUUCA | 11278 |
| 19 | UUCAUUCAUUCAUUCAUUC | 11279 |
| 20 | AUUCAUUCAUUCAUUCAUUC | 11280 |
| 20 | CAUUCAUUCAUUCAUUCAUU | 11281 |
| 20 | UCAUUCAUUCAUUCAUUCAU | 11282 |
| 20 | UUCAUUCAUUCAUUCAUUCA | 11283 |
| 21 | AUUCAUUCAUUCAUUCAUUCA | 11284 |
| 21 | CAUUCAUUCAUUCAUUCAUUC | 11285 |
| 21 | UCAUUCAUUCAUUCAUUCAUU | 11286 |
| 21 | UUCAUUCAUUCAUUCAUUCAU | 11287 |
| 22 | CAUUCAUUCAUUCAUUCAUUCA | 11288 |
| 22 | UCAUUCAUUCAUUCAUUCAUUC | 11289 |
| 22 | UUCAUUCAUUCAUUCAUUCAUU | 11290 |
| 23 | UCAUUCAUUCAUUCAUUCAUUCA | 11291 |
| 23 | UUCAUUCAUUCAUUCAUUCAUUC | 11292 |
| 24 | UUCAUUCAUUCAUUCAUUCAUUCA | 11293 |

TABLE XV

Synthetic Nucleic acid molecules

| | | | |
|---|---|---|---|
| RPI# | Alias | Sequence | SeqID |

| | | | |
|---|---|---|---|
| 24961 | HBV DR1 2′Oallyl P═S | gₛcₛaₛgₛaₛgₛgₛuₛgₛaₛaₛB | 11294 |
| 24997 | HBV DR1 2′Oallyl P═S control | aₛaₛgₛuₛgₛgₛaₛgₛaₛcₛgₛB | 11295 |
| | HBV 1866-1869 1 × 2′Oallyl | | |
| 24956 | P═S | uₛuₛcₛaₛB | 11296 |
| | HBV 1866-1869 1 × 2′Oallyl | | |
| 24992 | P═S control | aₛcₛuₛuₛB | 11297 |
| | HBV 1866-1869 2 × 2′Oallyl | | |
| 24941 | P═S | uₛuₛcₛaₛuₛuₛcₛaₛB | 11298 |
| | HBV 1866-1869 2 × 2′Oallyl | | |
| 24959 | P═S control | aₛcₛuₛuₛaₛcₛuₛuₛB | 11299 |
| | HBV 1866-1869 3 × 2′Oallyl | | |
| 24944 | P═S | uₛuₛcₛaₛuₛuₛcₛaₛuₛuₛcₛaₛB | 11300 |
| | HBV 1866-1869 3 × 2′Oallyl | | |
| 24962 | P═S control | aₛcₛuₛuₛaₛcₛuₛuₛaₛcₛuₛuₛB | 11301 |
| | HBV 1866-1869 4 × 2′Oallyl | | |
| 24945 | P═S | uₛuₛcₛaₛuₛuₛcₛaₛuₛuₛcₛaₛuₛuₛcₛaₛB | 11302 |
| | HBV 1866-1869 4 × 2′Oallyl | | |
| 24963 | P═S control | aₛcₛuₛuₛaₛcₛuₛuₛaₛcₛuₛuₛaₛcₛuₛuₛB | 11303 |
| 24938 | HBV 1866-1869 2′Oallyl P═S | uₛgₛaₛaₛB | 11304 |
| | HBV 1866-1869 2′Oallyl P═S | | |
| 24974 | control | aₛaₛgₛuₛB | 11305 |
| 24940 | HBV 1866-1872 2′Oallyl P═S | gₛcₛuₛuₛgₛaₛaₛB | 11306 |
| | HBV 1866-1872 2′Oallyl P═S | | |
| 24958 | control | aₛaₛgₛuₛuₛcₛgₛB | 11307 |
| 24943 | HBV 1866-1876 2′Oallyl P═S | gₛgₛaₛgₛgₛcₛuₛuₛgₛaₛaB | 11308 |
| | HBV 1866-1876 2′Oallyl P═S | | |
| 24979 | control | aₛaₛgₛuₛuₛcₛgₛgₛaₛgₛgₛB | 11309 |
| | | gₛaₛaₛauu cUGAuGaggccguuaggccGaa | |
| 18341 | HBV-273 UH.Rz-7 allyl stab1 | Agagaag B | 10887 |
| | HBV-273 UH.Rz-7 allyl stab1 | aₛaₛuₛgₛagg cUAGuGacgccguuaggcgGaa | |
| 24588 | inact3 scram1 (GUUA SAC) | Aaaugaa B | 11310 |
| 24929 | HBV 1866-1969 2′Omethyl | ugaaB | 11311 |
| | HBV 1866-1969 2′Omethyl | | |
| 24965 | control | aaguB | 11312 |
| 24934 | HBV 1866-1876 2′Omethyl | ggaggcuugaaB | 11313 |
| | HBV 1866-1876 2′Omethyl | | |
| 24970 | control | aaguucggaggB | 11314 |
| 24976 | HBV 1866-1872 2′Omethyl | gcuugaaB | 11315 |
| | HBV 1866-1872 2′Omethyl | | |
| 24949 | control | aaguucgB | 11316 |
| 24952 | HBV DR1 2′Omethyl | gcagaggugaaB | 11317 |
| 24988 | HBV DR1 2′Omethyl control | aaguggagacgB | 11318 |
| 24947 | HBV 1866-1869 1 × 2′Omethyl | uucaB | 11319 |
| | HBV 1866-1869 1 × 2′Omethyl | | |
| 24983 | control | acuuB | 11320 |
| 24986 | HBV 1866-1869 2 × 2′Omethyl | uucauucaB | 11321 |
| | HBV 1866-1869 2 × 2′Omethyl | | |
| 24950 | control | acuuacuuB | 11322 |
| 24989 | HBV 1866-1869 3 × 2′Omethyl | uucauucauucaB | 11323 |
| | HBV 1866-1869 3 × 2′Omethyl | | |
| 24953 | control | acuuacuuacuuB | 11324 |
| 24936 | HBV 1866-1869 4 × 2′Omethyl | uucauucauucauucaB | 11325 |
| | HBV 1866-1869 4 × 2′Omethyl | | |
| 24954 | control | acuuacuuacuuacuuB | 11326 |
| 25639 | HBV 5′ EnI pos OMe P═S | B uₛuₛuₛcₛuₛaₛaₛgₛuₛaₛaₛaₛcₛaₛgₛu B | 11327 |
| 25640 | HBV 5′ EnI neg OMe P═S | B aₛcₛuₛgₛuₛuₛuₛaₛcₛuₛuₛaₛgₛaₛaₛa B | 11328 |
| 25641 | HBV 5′ EnI sc OMe P═S | B aₛaₛgₛuₛaₛaₛcₛuₛcₛuₛaₛuₛgₛuₛuₛa B | 11329 |
| | | B | |
| | | uₛaₛcₛaₛuₛgₛaₛaₛcₛcₛuₛuₛuₛaₛcₛcₛcₛc | |
| 25642 | HBV 3′ EnI pos OMe P═S | B | 11330 |
| | | B gₛgₛgₛuₛaₛaₛaₛgₛgₛuₛuₛcₛaₛuₛgₛuₛa | |
| 25643 | HBV 3′ EnI neg OMe P═S | B | 11331 |
| | | B | |
| | | aₛcₛcₛuₛaₛuₛcₛgₛcₛcₛuₛaₛcₛuₛcₛuₛaₛa | |
| 25644 | HBV 3′ EnI pos sc OMe P═S | B | 11332 |
| | | B uₛgₛaₛuₛaₛgₛcₛgₛgₛaₛuₛgₛaₛgₛaₛuₛu | |
| 25645 | HBV 5′ EnI neg sc OMe P═S | B | 11333 |
| 25646 | HBV DR1 pos OMe P═s | B uₛuₛcₛaₛcₛcₛuₛcₛuₛgₛc B | 11334 |
| 25651 | HBV 5′ EnI pos Oallyl P═S | B uₛuₛuₛcₛuₛaₛaₛgₛuₛaₛaₛaₛcₛaₛgₛu B | 11335 |
| 25652 | HBV 5′ EnI neg Oallyl P═S | B aₛcₛuₛgₛuₛuₛuₛaₛcₛuₛuₛaₛgₛaₛaₛa B | 11336 |
| 25653 | HBV 5′ EnI sc Oallyl P═S | B aₛaₛgₛuₛaₛaₛcₛuₛcₛuₛaₛuₛgₛuₛuₛa B | 11337 |
| | | B | |
| | | uₛaₛcₛaₛuₛgₛaₛaₛcₛcₛuₛuₛuₛaₛcₛcₛcₛc | |
| 25654 | HBV 3′ EnI pos Oallyl P═S | B | 11338 |
| | | B gₛgₛgₛuₛaₛaₛaₛgₛgₛuₛuₛcₛaₛuₛgₛuₛa | |
| 25655 | HBV 3′ EnI neg Oallyl P═S | B | 11339 |
| | | B | |
| | | aₛcₛcₛuₛaₛuₛcₛgₛcₛcₛuₛaₛcₛuₛcₛuₛaₛa | |
| 25656 | HBV 3′ EnI pos sc Oallyl P═S | B | 11340 |
| | | B uₛgₛaₛuₛaₛgₛcₛgₛgₛaₛuₛgₛaₛgₛaₛuₛu | |
| 25657 | HBV 5′ EnI neg sc Oallyl P═S | B | 11341 |
| 25658 | HBV DR1 pos Oallyl P═S | B uₛuₛcₛaₛcₛcₛuₛcₛuₛgₛc B | 11342 |

a, g, c, u = all 2′-O-allyl
a, g, c, u = 2′-O-methyl
U = 2′-C-allyl Uridine
S = phosphorothioate
B = inverted deoxyabasic

TABLE XVI
Comparison of Tumor Weight to HBV
DNA concentration in mice
inoculated with HepG2.2.15 cells

| Time point | HBV DNA | Tumor weight |
|---|---|---|
| (days) | copies/mL serum | (milligrams) |
| 1 | Below detection | No tumor |
| 1 | Below detection | No tumor |
| 1 | Below detection | No tumor |
| 1 | Below detection | No tumor |
| 7 | Below detection | No tumor |
| 7 | Below detection | No tumor |
| 7 | Below detection | No tumor |
| 7 | Below detection | No tumor |
| 14 | Below detection | No tumor |
| 14 | Below detection | No tumor |
| 14 | Below detection | No tumor |
| 14 | Below detection | No tumor |
| 35 | 356 | 33 |
| 35 | 125083 | 167 |
| 35 | 578 | No tumor |
| 35 | 386 | 56 |
| 42 | 493 | No tumor |
| 42 | 114431 | 790 |
| 42 | 94025 | 359 |
| 42 | 111882 | 647 |
| 49 | 189885 | 816 |
| 49 | Below detection | No tumor |
| 49 | 293 | 90 |
| 49 | 41477 | 2521 |

TABLE XVII
Comparison of Tumor Weight to
HBV DNA concentration in mice
inoculated with G418 resistant HepG2.2.15 cells

| Time point | HBV DNA copies/mL | Tumor weight |
|---|---|---|
| (days) | serum | (milligrams) |
| 37 | 7000 | 1120.0 |
| 37 | no sample | no sample |
| 37 | 400000 | 1962.3 |
| 37 | 26000 | 558.5 |
| 37 | 380000 | 2286.0 |
| 37 | 100 | 317.2 |
| 37 | 52000 | 1429.0 |
| 37 | 100 | 427.4 |
| 37 | 26000 | 813.2 |
| 37 | 1400 | 631.6 |
| 37 | 186000 | 1101.5 |
| 37 | 134000 | 1573.0 |
| 37 | 17800 | 1040.0 |
| 37 | 16600 | 1327.2 |
| 37 | 8200 | 275.7 |
| 37 | 68000 | 632.8 |
| 37 | 24000 | 1090.0 |
| 37 | 58000 | 1082.7 |
| 37 | 12400 | 1116.3 |
| 37 | 100 | 763.3 |

TABLE XVIII

HCV DNAzyme and Substrate Sequence

| | | | | |
|---|---|---|---|---|
| | | SEQ | | SEQ |
| Pos | Substrate | ID | DNAZYME | ID |

| | | | | |
|---|---|---|---|---|
| 10 | UGGGGGCG A CACUCCAC | 2594 | GTGGAGTG GGCTAGCTACAACGA CGCCCCCA | 11343 |
| 12 | GGGGCGAC A CUCCACCA | 2595 | TGGTGGAG GCCTAGCTACAACGA GTCGCCCC | 11344 |
| 17 | GACACUCC A CCAUAGAU | 2596 | ATCTATGG GGCTAGCTACAACGA CGAGTGTC | 11345 |
| 20 | ACUCCACC A UAGAUCAC | 2597 | GTGATCTA GGCTAGCTACAACGA GGTGGAGT | 11346 |
| 24 | CACCAUAG A UCACUCCC | 2598 | GGGAGTGA GGCTAGCTACAACGA CTATGGTG | 11347 |
| 27 | CAUAGAUC A CUCCCCUG | 2599 | CAGGGGAG GGCTAGCTACAACGA GATCTATG | 11348 |
| 35 | ACUCCCCU G UGAGGAAC | 2600 | GTTCCTCA GGCTAGCTACAACGA AGGGGAGT | 11349 |
| 42 | UGUGAGGA A CUACUGUC | 2601 | GACAGTAG GGCTAGCTACAACGA TCCTCACA | 11350 |
| 45 | GAGGAACU A CUGUCUUC | 2602 | GAAGACAG GGCTAGCTACAACGA AGTTCCTC | 11351 |
| 48 | GAACUACU G UCUUCACG | 2603 | CGTGAAGA GGCTAGCTACAACGA AGTAGTTC | 11352 |
| 54 | CUGUCUUC A CGCAGAAA | 2604 | TTTCTGCG GGCTAGCTACAACGA GAAGACAG | 11353 |
| 56 | GUCUUCAC G CAGAAAGC | 2605 | GCTTTCTG GGCTAGCTACAACGA GTGAAGAC | 11354 |
| 63 | CGCAGAAA G CGUCUAGC | 2606 | GCTAGACG GGCTAGCTACAACGA TTTCTGCG | 11355 |
| 65 | CAGAAAGC G UCUAGCCA | 2607 | TGGCTAGA GGCTAGCTACAACGA GCTTTCTG | 11356 |
| 70 | AGCGUCUA G CCAUGGCG | 2608 | CGCCATGG GGCTAGCTACAACGA TAGACGCT | 11357 |
| 73 | GUCUAGCC A UGGCGUUA | 2609 | TAACGCCA GGCTAGCTACAACGA GGCTAGAC | 11358 |
| 76 | UAGCCAUG G CGUUAGUA | 2610 | TACTAACG GGCTAGCTACAACGA CATGGCTA | 11359 |
| 78 | GCCAUGGC G UUAGUAUG | 2611 | CATACTAA GGCTAGCTACAACGA GCCATGGC | 11360 |
| 82 | UGGCGUUA G UAUGAGUG | 2612 | CACTCATA GGCTAGCTACAACGA TAACGCCA | 11361 |
| 84 | GCGUUAGU A UGAGUGUC | 2613 | GACACTCA GGCTAGCTACAACGA ACTAACGC | 11362 |
| 88 | UAGUAUGA G UGUCGUGC | 2614 | GCACGACA GGCTAGCTACAACGA TCATACTA | 11363 |
| 90 | GUAUGAGU G UCGUGCAG | 2615 | CTGCACGA GGCTAGCTACAACGA ACTCATAC | 11364 |
| 93 | UGAGUGUC G UGCAGCCU | 2616 | AGGCTGCA GGCTAGCTACAACGA GACACTCA | 11365 |
| 95 | AGUGUCGU G CAGCCUCC | 2617 | GGAGGCTG GGCTAGCTACAACGA ACGACACT | 11366 |
| 98 | GUCGUGCA G CCUCCAGG | 2618 | CCTGGAGG GGCTAGCTACAACGA TGCACGAC | 11367 |
| 107 | CCUCCAGG A CCCCCCCU | 2619 | AGGGGGGG GGCTAGCTACAACGA CCTGGAGG | 11368 |
| 125 | CCGGGAGA G CCAUAGUG | 2620 | CACTATGG GGCTAGCTACAACGA TCTCCCGG | 11369 |
| 128 | GGAGAGCC A UAGUGGUC | 2621 | GACCACTA GGCTAGCTACAACGA GGCTCTCC | 11370 |
| 131 | GAGCCAUA G UGGUCUGC | 2622 | GCAGACCA GGCTAGCTACAACGA TATGGCTC | 11371 |
| 134 | CCAUAGUG G UCUGCGGA | 2623 | TCCGCAGA GGCTAGCTACAACGA CACTATGG | 11372 |
| 138 | AGUGGUCU G CGGAACCG | 2624 | CGGTTCCG GGCTAGCTACAACGA AGACCACT | 11373 |
| 143 | UCUGCGGA A CCGGUGAG | 2625 | CTCACCGG GGCTAGCTACAACGA TCCGCAGA | 11374 |
| 147 | CGGAACCG G UGAGUACA | 2626 | TGTACTCA GGCTAGCTACAACGA CGGTTCCG | 11375 |
| 151 | ACCGGUGA G UACACCGG | 2627 | CCGGTGTA GGCTAGCTACAACGA TCACCGGT | 11376 |
| 153 | CGGUGAGU A CACCGGAA | 2628 | TTCCGGTG GGCTAGCTACAACGA ACTCACCG | 11377 |
| 155 | GUGAGUAC A CCGGAAUU | 2629 | AATTCCGG GGCTAGCTACAACGA GTACTCAC | 11378 |
| 161 | ACACCGGA A UUGCCAGG | 2630 | CCTGGCAA GGCTAGCTACAACGA TCCGGTGT | 11379 |
| 164 | CCGGAAUU G CCAGGACG | 2631 | CGTCCTGG GGCTAGCTACAACGA AATTCCGG | 11380 |
| 170 | UUGCCAGG A CGACCGGG | 2632 | CCCGGTCG GGCTAGCTACAACGA CCTGGCAA | 11381 |
| 173 | CCAGGACG A CCGGGUCC | 2633 | GGACCCGG GGCTAGCTACAACGA CGTCCTGG | 11382 |
| 178 | ACGACCGG G UCCUUUCU | 2634 | AGAAAGGA GGCTAGCTACAACGA CCGGTCGT | 11383 |
| 190 | UUUCUUGG A UCAACCCG | 2635 | CGGGTTGA GGCTAGCTACAACGA CCAAGAAA | 11384 |
| 194 | UUGGAUCA A CCCGCUCA | 2636 | TGAGCGGG GGCTAGCTACAACGA TGATCCAA | 11385 |
| 198 | AUCAACCC G CUCAAUGC | 2637 | GCATTGAG GGCTAGCTACAACGA GGGTTGAT | 11386 |
| 203 | CCCGCUCA A UGCCUGGA | 2638 | TCCAGGCA GGCTAGCTACAACGA TGAGCGGG | 11387 |
| 205 | CGCUCAAU G CCUGGAGA | 2639 | TCTCCAGG GGCTAGCTACAACGA ATTGAGCG | 11388 |
| 213 | GCCUGGAG A UUUGGGCG | 2640 | CGCCCAAA GGCTAGCTACAACGA CTCCAGGC | 11389 |
| 219 | AGAUUUGG G CGUGCCCC | 2641 | GGGGCACG GGCTAGCTACAACGA CCAAATCT | 11390 |
| 221 | AUUUGGGC G UGCCCCCG | 2642 | CGGGGGCA GGCTAGCTACAACGA GCCCAAAT | 11391 |
| 223 | UUGGGCGU G CCCCCGCG | 2643 | CGCGGGGG GGCTAGCTACAACGA ACGCCCAA | 11392 |
| 229 | GUGCCCCC G CGAGACUG | 2644 | CAGTCTCG GGCTAGCTACAACGA GGGGGCAC | 11393 |
| 234 | CCCGCGAG A CUGCUAGC | 2645 | GCTAGCAG GGCTAGCTACAACGA CTCGCGGG | 11394 |
| 237 | GCGAGACU G CUAGCCGA | 2646 | TCGGCTAG GGCTAGCTACAACGA AGTCTCGC | 11395 |
| 241 | GACUGCUA G CCGAGUAG | 2647 | CTACTCGG GGCTAGCTACAACGA TAGCAGTC | 11396 |
| 246 | CUAGCCGA G UAGUGUUG | 2648 | CAACACTA GGCTAGCTACAACGA TCGGCTAG | 11397 |
| 249 | GCCGAGUA G UGUUGGGU | 2649 | ACCCAACA GGCTAGCTACAACGA TACTCGGC | 11398 |
| 251 | CGAGUAGU G UUGGGUCG | 2650 | CGACCCAA GGCTAGCTACAACGA ACTACTCG | 11399 |
| 256 | AGUGUUGG G UCGCGAAA | 2651 | TTTCGCGA GGCTAGCTACAACGA CCAACACT | 11400 |
| 259 | GUUGGGUC G CGAAAGGC | 2652 | GCCTTTCG GGCTAGCTACAACGA GACCCAAC | 11401 |
| 266 | CGCGAAAG G CCUUGUGG | 2653 | CCACAAGG GGCTAGCTACAACGA CTTTCGCG | 11402 |
| 271 | AAGGCCUU G UGGUACUG | 2654 | CAGTACCA GGCTAGCTACAACGA AAGGCCTT | 11403 |
| 274 | GCCUUGUG G UACUGCCU | 2655 | AGGCAGTA GGCTAGCTACAACGA CACAAGGC | 11404 |
| 276 | CUUGUGGU A CUGCCUGA | 2656 | TCAGGCAG GGCTAGCTACAACGA ACCACAAG | 11405 |
| 279 | GUGGUACU G CCUGAUAG | 2657 | CTATCAGG GGCTAGCTACAACGA AGTACCAC | 11406 |
| 284 | ACUGCCUG A UAGGGUGC | 2658 | GCACCCTA GGCTAGCTACAACGA CAGGCAGT | 11407 |
| 289 | CUGAUAGG G UGCUUGCG | 2659 | CGCAAGCA GGCTAGCTACAACGA CCTATCAG | 11408 |
| 291 | GAUAGGGU G CUUGCGAG | 2660 | CTCGCAAG GGCTAGCTACAACGA ACCCTATC | 11409 |
| 295 | GGGUGCUU G CGAGUGCC | 2661 | GGCACTCG GGCTAGCTACAACGA AAGCACCC | 11410 |
| 299 | GCUUGCGA G UGCCCCGG | 2662 | CCGGGGCA GGCTAGCTACAACGA TCGCAAGC | 11411 |
| 301 | UUGCGAGU G CCCCGGGA | 2663 | TCCCGGGG GGCTAGCTACAACGA ACTCGCAA | 11412 |
| 311 | CCCGGGAG G UCUCGUAG | 2664 | CTACGAGA GGCTAGCTACAACGA CTCCCGGG | 11413 |
| 316 | GAGGUCUC G UAGACCGU | 2665 | ACGGTCTA GGCTAGCTACAACGA GAGACCTC | 11414 |
| 320 | UCUCGUAG A CCGUGCAC | 2666 | GTGCACGG GGCTAGCTACAACGA CTACGAGA | 11415 |
| 323 | CGUAGACC G UGCACCAU | 2667 | ATGGTGCA GGCTAGCTACAACGA GGTCTACG | 11416 |
| 325 | UAGACCGU G CACCAUGA | 2668 | TCATGGTG GGCTAGCTACAACGA ACGGTCTA | 11417 |
| 327 | GACCGUGC A CCAUGAGC | 2669 | GCTCATGG GGCTAGCTACAACGA GCACGGTC | 11418 |
| 330 | CGUGCACC A UGAGCACG | 2670 | CGTGCTCA GGCTAGCTACAACGA GGTGCACG | 11419 |
| 334 | CACCAUGA G CACGAAUC | 2671 | GATTCGTG GGCTAGCTACAACGA TCATGGTG | 11420 |
| 336 | CCAUGAGC A CGAAUCCU | 2672 | AGGATTCG GGCTAGCTACAACGA GCTCATGG | 11421 |
| 340 | GAGCACGA A UCCUAAAC | 2673 | GTTTAGGA GGCTAGCTACAACGA TCGTGCTC | 11422 |
| 347 | AAUCCUAA A CCUCAAAG | 2674 | CTTTGAGG GGCTAGCTACAACGA TTAGGATT | 11423 |
| 360 | AAAGAAAA A CCAAACGU | 2675 | ACGTTTGG GGCTAGCTACAACGA TTTTCTTT | 11424 |
| 365 | AAAACCAA A CGUAACAC | 2676 | GTGTTACG GGCTAGCTACAACGA TTGGTTTT | 11425 |
| 367 | AACCAAAC G UAACACCA | 2677 | TGGTGTTA GGCTAGCTACAACGA GTTTGGTT | 11426 |
| 370 | CAAACGUA A CACCAACC | 2678 | GGTTGGTG GGCTAGCTACAACGA TACGTTTG | 11427 |
| 372 | AACGUAAC A CCAACCGC | 2679 | GCGGTTGG GGCTAGCTACAACGA GTTACGTT | 11428 |
| 376 | UAACACCA A CCGCCGCC | 2680 | GGCGGCGG GGCTAGCTACAACGA TGGTGTTA | 11429 |
| 379 | CACCAACC G CCGCCCAC | 2681 | GTGGGCGG GGCTAGCTACAACGA GGTTGGTG | 11430 |
| 382 | CAACCGCC G CCCACAGG | 2682 | CCTGTGGG GGCTAGCTACAACGA GGCGGTTG | 11431 |
| 386 | CGCCGCCC A CAGGACGU | 2683 | ACGTCCTG GGCTAGCTACAACGA GGGCGGCG | 11432 |
| 391 | CCCACAGG A CGUCAAGU | 2684 | ACTTGACG GGCTAGCTACAACGA CCTGTGGG | 11433 |
| 393 | CACAGGAC G UCAAGUUC | 2685 | GAACTTGA GGCTAGCTACAACGA GTCCTGTG | 11434 |
| 398 | GACGUCAA G UUCCCGGG | 2686 | CCCGGGAA GGCTAGCTACAACGA TTGACGTC | 11435 |
| 406 | GUUCCCGG G CGGUGGUC | 2687 | GACCACCG GGCTAGCTACAACGA CCGGGAAC | 11436 |
| 409 | CCCGGGCG G UGGUCAGA | 2688 | TCTGACCA GGCTAGCTACAACGA CGCCCGGG | 11437 |
| 412 | GGGCGGUG G UCAGAUCG | 2689 | CGATCTGA GGCTAGCTACAACGA CACCGCCC | 11438 |
| 417 | GUGGUCAG A UCGUUGGU | 2690 | ACCAACGA GGCTAGCTACAACGA CTGACCAC | 11439 |
| 420 | GUCAGAUC G UUGGUGGA | 2691 | TCCACCAA GGCTAGCTACAACGA GATCTGAC | 11440 |
| 424 | GAUCGUUG G UGGAGUUU | 2692 | AAACTCCA GGCTAGCTACAACGA CAACGATC | 11441 |
| 429 | UUGGUGGA G UUUACCUG | 2693 | CAGGTAAA GGCTAGCTACAACGA TCCACCAA | 11442 |
| 433 | UGGAGUUU A CCUGUUGC | 2694 | GCAACAGG GGCTAGCTACAACGA AAACTCCA | 11443 |
| 437 | GUUUACCU G UUGCCGCG | 2695 | CGCGGCAA GGCTAGCTACAACGA AGGTAAAC | 11444 |
| 440 | UACCUGUU G CCGCGCAG | 2696 | CTGCGCGG GGCTAGCTACAACGA AACAGGTA | 11445 |
| 443 | CUGUUGCC G CGCAGGGG | 2697 | CCCCTGCG GGCTAGCTACAACGA GGCAACAG | 11446 |
| 445 | GUUGCCGC G CAGGGGCC | 2698 | GGCCCCTG GGCTAGCTACAACGA GCGGCAAC | 11447 |
| 451 | GCGCAGGG G CCCCAGGU | 2699 | ACCTGGGG GGCTAGCTACAACGA CCCTGCGC | 11448 |
| 458 | GGCCCCAG G UUGGGUGU | 2700 | ACACCCAA GGCTAGCTACAACGA CTGGGGCC | 11449 |
| 463 | CAGGUUGG G UGUGCGCG | 2701 | CGCGCACA GGCTAGCTACAACGA CCAACCTG | 11450 |
| 465 | GGUUGGGU G UGCGCGCG | 2702 | CGCGCGCA GGCTAGCTACAACGA ACCCAACC | 11451 |
| 467 | UUGGGUGU G CGCGCGAC | 2703 | GTCGCGCG GGCTAGCTACAACGA ACACCCAA | 11452 |
| 469 | GGGUGUGC G CGCGACUA | 2704 | TAGTCGCG GGCTAGCTACAACGA GCACACCC | 11453 |
| 471 | GUGUGCGC G CGACUAGG | 2705 | CCTAGTCG GGCTAGCTACAACGA GCGCACAC | 11454 |
| 474 | UGCGCGCG A CUAGGAAG | 2706 | CTTCCTAG GGCTAGCTACAACGA CGCGCGCA | 11455 |
| 483 | CUAGGAAG A CUUCCGAG | 2707 | CTCGGAAG GGCTAGCTACAACGA CTTCCTAG | 11456 |
| 491 | ACUUCCGA G CGGUCGCA | 2708 | TGCGACCG GGCTAGCTACAACGA TCGGAAGT | 11457 |
| 494 | UCCGAGCG G UCGCAACC | 2709 | GGTTGCGA GGCTAGCTACAACGA CGCTCGGA | 11458 |
| 497 | GAGCGGUC G CAACCUCG | 2710 | CGAGGTTG GGCTAGCTACAACGA GACCGCTC | 11459 |
| 500 | CGGUCGCA A CCUCGUGG | 2711 | CCACGAGG GGCTAGCTACAACGA TGCGACCG | 11460 |
| 505 | GCAACCUC G UGGAAGGC | 2712 | GCCTTCCA GGCTAGCTACAACGA GAGGTTGC | 11461 |
| 512 | CGUGGAAG G CGACAACC | 2713 | GGTTGTCG GGCTAGCTACAACGA CTTCCACG | 11462 |
| 515 | GGAAGGCG A CAACCUAU | 2714 | ATAGGTTG GGCTAGCTACAACGA CGCCTTCC | 11463 |
| 518 | AGGCGACA A CCUAUCCC | 2715 | GGGATAGG GGCTAGCTACAACGA TGTCGCCT | 11464 |
| 522 | GACAACCU A UCCCCAAG | 2716 | CTTGGGGA GGCTAGCTACAACGA AGGTTGTC | 11465 |
| 531 | UCCCCAAG G CUCGCCGG | 2717 | CCGGCGAG GGCTAGCTACAACGA CTTGGGGA | 11466 |
| 535 | CAAGGCUC G CCGGCCCG | 2718 | CGGGCCGG GGCTAGCTACAACGA GAGCCTTG | 11467 |
| 539 | GCUCGCCG G CCCGAGGG | 2719 | CCCTCGGG GGCTAGCTACAACGA CGGCGAGC | 11468 |
| 547 | GCCCGAGG G CAGGGCCU | 2720 | AGGCCCTG GGCTAGCTACAACGA CCTCGGGC | 11469 |
| 552 | AGGGCAGG G CCUGGGCU | 2721 | AGCCCAGG GGCTAGCTACAACGA CCTGCCCT | 11470 |
| 558 | GGGCCUGG G CUCAGCCC | 2722 | GGGCTGAG GGCTAGCTACAACGA CCAGGCCC | 11471 |
| 563 | UGGGCUCA G CCCGGGUA | 2723 | TACCCGGG GGCTAGCTACAACGA TGAGCCCA | 11472 |
| 569 | CAGCCCGG G UACCCUUG | 2724 | CAAGGGTA GGCTAGCTACAACGA CCGGGCTG | 11473 |
| 571 | GCCCGGGU A CCCUUGGC | 2725 | GCCAAGGG GGCTAGCTACAACGA ACCCGGGC | 11474 |
| 578 | UACCCUUG G CCCCUCUA | 2726 | TAGAGGGG GGCTAGCTACAACGA CAAGGGTA | 11475 |
| 586 | GCCCCUCU A UGGCAAUG | 2727 | CATTGCCA GGCTAGCTACAACGA AGAGGGGC | 11476 |
| 589 | CCUCUAUG G CAAUGAGG | 2728 | CCTCATTG GGCTAGCTACAACGA CATAGAGG | 11477 |
| 592 | CUAUGGCA A UGAGGGCU | 2729 | AGCCCTCA GGCTAGCTACAACGA TGCCATAG | 11478 |
| 598 | CAAUGAGG G CUUAGGGU | 2730 | ACCCTAAG GGCTAGCTACAACGA CCTCATTG | 11479 |
| 605 | GGCUUAGG G UGGGCAGG | 2731 | CCTGCCCA GGCTAGCTACAACGA CCTAAGCC | 11480 |
| 609 | UAGGGUGG G CAGGAUGG | 2732 | CCATCCTG GGCTAGCTACAACGA CCACCCTA | 11481 |
| 614 | UGGGCAGG A UGGCUCCU | 2733 | AGGAGCCA GGCTAGCTACAACGA CCTGCCCA | 11482 |
| 617 | GCAGGAUG G CUCCUGUC | 2734 | GACAGGAG GGCTAGCTACAACGA CATCCTGC | 11483 |
| 623 | UGGCUCCU G UCACCCCG | 2735 | CGGGGTGA GGCTAGCTACAACGA AGGAGCCA | 11484 |
| 626 | CUCCUGUC A CCCCGCGG | 2736 | CCGCGGGG GGCTAGCTACAACGA GACAGGAG | 11485 |
| 631 | GUCACCCC G CGGCUCCC | 2737 | GGGAGCCG GGCTAGCTACAACGA GGGGTGAC | 11486 |
| 634 | ACCCCGCG G CUCCCGGC | 2738 | GCCGGGAG GGCTAGCTACAACGA CGCGGGGT | 11487 |
| 641 | GGCUCCCG G CCUAGUUG | 2739 | CAACTAGG GGCTAGCTACAACGA CGGGAGCC | 11488 |
| 646 | CCGGCCUA G UUGGGGCC | 2740 | GGCCCCAA GGCTAGCTACAACGA TAGGCCGG | 11489 |
| 652 | UAGUUGGG G CCCCACGG | 2741 | CCGTGGGG GGCTAGCTACAACGA CCCAACTA | 11490 |
| 657 | GGGGCCCC A CGGACCCC | 2742 | GGGGTCCG GGCTAGCTACAACGA GGGGCCCC | 11491 |
| 661 | CCCCACGG A CCCCCGGC | 2743 | GCCGGGGG GGCTAGCTACAACGA CCGTGGGG | 11492 |
| 668 | GACCCCCG G CGUAGGUC | 2744 | GACCTACG GGCTAGCTACAACGA CGGGGGTC | 11493 |
| 670 | CCCCCGGC G UAGGUCGC | 2745 | GCGACCTA GGCTAGCTACAACGA GCCGGGGG | 11494 |
| 674 | CGGCGUAG G UCGCGUAA | 2746 | TTACGCGA GGCTAGCTACAACGA CTACGCCG | 11495 |
| 677 | CGUAGGUC G CGUAACUU | 2747 | AAGTTACG GGCTAGCTACAACGA GACCTACG | 11496 |
| 679 | UAGGUCGC G UAACUUGG | 2748 | CCAAGTTA GGCTAGCTACAACGA GCGACCTA | 11497 |
| 682 | GUCGCGUA A CUUGGGUA | 2749 | TACCCAAG GGCTAGCTACAACGA TACGCGAC | 11498 |
| 688 | UAACUUGG G UAAGGUCA | 2750 | TGACCTTA GGCTAGCTACAACGA CCAAGTTA | 11499 |
| 693 | UGGGUAAG G UCAUCGAU | 2751 | ATCGATGA GGCTAGCTACAACGA CTTACCCA | 11500 |
| 696 | GUAAGGUC A UCGAUACC | 2752 | GGTATCGA GGCTAGCTACAACGA GACCTTAC | 11501 |
| 700 | GGUCAUCG A UACCCUCA | 2753 | TGAGGGTA GGCTAGCTACAACGA CGATGACC | 11502 |
| 702 | UCAUCGAU A CCCUCACA | 2754 | TGTGAGGG GGCTAGCTACAACGA ATCGATGA | 11503 |
| 708 | AUACCCUC A CAUGCGGC | 2755 | GCCGCATG GGCTAGCTACAACGA GAGGGTAT | 11504 |
| 710 | ACCCUCAC A UGCGGCUU | 2756 | AAGCCGCA GGCTAGCTACAACGA GTGAGGGT | 11505 |
| 712 | CCUCACAU G CGGCUUCG | 2757 | CGAAGCCG GGCTAGCTACAACGA ATGTGAGG | 11506 |
| 715 | CACAUGCG G CUUCGCCG | 2758 | CGGCGAAG GGCTAGCTACAACGA CGCATGTG | 11507 |
| 720 | GCGGCUUC G CCGACCUC | 2759 | GAGGTCGG GGCTAGCTACAACGA GAAGCCGC | 11508 |
| 724 | CUUCGCCG A CCUCAUGG | 2760 | CCATGAGG GGCTAGCTACAACGA CGGCGAAG | 11509 |
| 729 | CCGACCUC A UGGGGUAC | 2761 | GTACCCCA GGCTAGCTACAACGA GAGGTCGG | 11510 |
| 734 | CUCAUGGG G UACAUUCC | 2762 | GGAATGTA GGCTAGCTACAACGA CCCATGAG | 11511 |
| 736 | CAUGGGGU A CAUUCCGC | 2763 | GCGGAATG GGCTAGCTACAACGA ACCCCATG | 11512 |
| 738 | UGGGGUAC A UUCCGCUC | 2764 | GAGCGGAA GGCTAGCTACAACGA GTACCCCA | 11513 |
| 743 | UACAUUCC G CUCGUCGG | 2765 | CCGACGAG GGCTAGCTACAACGA GGAATGTA | 11514 |
| 747 | UUCCGCUC G UCGGCGCC | 2766 | GGCGCCGA GGCTAGCTACAACGA GAGCGGAA | 11515 |
| 751 | GCUCGUCG G CGCCCCCU | 2767 | AGGGGGCG GGCTAGCTACAACGA CGACGAGC | 11516 |
| 753 | UCGUCGGC G CCCCCUUG | 2768 | CAAGGGGG GGCTAGCTACAACGA GCCGACGA | 11517 |
| 766 | CUUGGGAG G CACUGCCA | 2769 | TGGCAGTG GGCTAGCTACAACGA CTCCCAAG | 11518 |
| 768 | UGGGAGGC A CUGCCAGG | 2770 | CCTGGCAG GGCTAGCTACAACGA GCCTCCCA | 11519 |
| 771 | GAGGCACU G CCAGGGCC | 2771 | GGCCCTGG GGCTAGCTACAACGA AGTGCCTC | 11520 |
| 777 | CUGCCAGG G CCCUGGCG | 2772 | CGCCAGGG GGCTAGCTACAACGA CCTGGCAG | 11521 |
| 783 | GGGCCCUG G CGCAUGGC | 2773 | GCCATGCG GGCTAGCTACAACGA CAGGGCCC | 11522 |
| 785 | GCCCUGGC G CAUGGCGU | 2774 | ACGCCATG GGCTAGCTACAACGA GCCAGGGC | 11523 |
| 787 | CCUGGCGC A UGGCGUCC | 2775 | GGACGCCA GGCTAGCTACAACGA GCGCCAGG | 11524 |
| 790 | GGCGCAUG G CGUCCGGG | 2776 | CCCGGACG GGCTAGCTACAACGA CATGCGCC | 11525 |
| 792 | CGCAUGGC G UCCGGGUU | 2777 | AACCCGGA GGCTAGCTACAACGA GCCATGCG | 11526 |
| 798 | GCGUCCGG G UUCUGGAA | 2778 | TTCCAGAA GGCTAGCTACAACGA CCGGACGC | 11527 |
| 808 | UCUGGAAG A CGGCGUGA | 2779 | TCACGCCG GGCTAGCTACAACGA CTTCCAGA | 11528 |
| 811 | GGAAGACG G CGUGAACU | 2780 | AGTTCACG GGCTAGCTACAACGA CGTCTTCC | 11529 |
| 813 | AAGACGGC G UGAACUAU | 2781 | ATAGTTCA GGCTAGCTACAACGA GCCGTCTT | 11530 |
| 817 | CGGCGUGA A CUAUGCAA | 2782 | TTGCATAG GGCTAGCTACAACGA TCACGCCG | 11531 |
| 820 | CGUGAACU A UGCAACAG | 2783 | CTGTTGCA GGCTAGCTACAACGA AGTTCACG | 11532 |
| 822 | UGAACUAU G CAACAGGG | 2784 | CCCTGTTG GGCTAGCTACAACGA ATAGTTCA | 11533 |
| 825 | ACUAUGCA A CAGGGAAU | 2785 | ATTCCCTG GGCTAGCTACAACGA TGCATAGT | 11534 |
| 832 | AACAGGGA A UCUGCCCG | 2786 | CGGGCAGA GGCTAGCTACAACGA TCCCTGTT | 11535 |
| 836 | GGGAAUCU G CCCGGUUG | 2787 | CAACCGGG GGCTAGCTACAACGA AGATTCCC | 11536 |
| 841 | UCUGCCCG G UUGCUCUU | 2788 | AAGAGCAA GGCTAGCTACAACGA CGGGCAGA | 11537 |
| 844 | GCCCGGUU G CUCUUUCU | 2789 | AGAAAGAG GGCTAGCTACAACGA AACCGGGC | 11538 |
| 855 | CUUUCUCU A UCUUCCUC | 2790 | GAGGAAGA GGCTAGCTACAACGA AGAGAAAG | 11539 |
| 867 | UCCUCUUG G CUCUGCUG | 2791 | CAGCAGAG GGCTAGCTACAACGA CAAGAGGA | 11540 |
| 872 | UUGGCUCU G CUGCCCUG | 2792 | CAGGGCAG GGCTAGCTACAACGA AGAGCCAA | 11541 |
| 875 | GCUCUGCU G CCCUGUCU | 2793 | AGACAGGG GGCTAGCTACAACGA AGCAGAGC | 11542 |
| 880 | GCUGCCCU G UCUGACCA | 2794 | TGGTCAGA GGCTAGCTACAACGA AGGGCAGC | 11543 |
| 885 | CCUGUCUG A CCAUCCCA | 2795 | TGGGATGG GGCTAGCTACAACGA CAGACAGG | 11544 |
| 888 | GUCUGACC A UCCCAGCC | 2796 | GGCTGGGA GGCTAGCTACAACGA GGTCAGAC | 11545 |
| 894 | CCAUCCCA G CCUCCGCU | 2797 | AGCGGAGG GGCTAGCTACAACGA TGGGATGG | 11546 |
| 900 | CAGCCUCC G CUUAUGAG | 2798 | CTCATAAG GGCTAGCTACAACGA GGAGGCTG | 11547 |
| 904 | CUCCGCUU A UGAGGUGU | 2799 | ACACCTCA GGCTAGCTACAACGA AAGCGGAG | 11548 |
| 909 | CUUAUGAG G UGUGCAAC | 2800 | GTTGCACA GGCTAGCTACAACGA CTCATAAG | 11549 |
| 911 | UAUGAGGU G UGCAACGC | 2801 | GCGTTGCA GGCTAGCTACAACGA ACCTCATA | 11550 |
| 913 | UGAGGUGU G CAACGCGU | 2802 | ACGCGTTG GGCTAGCTACAACGA ACACCTCA | 11551 |
| 916 | GGUGUGCA A CGCGUCCG | 2803 | CGGACGCG GGCTAGCTACAACGA TGCACACC | 11552 |
| 918 | UGUGCAAC G CGUCCGGG | 2804 | CCCGGACG GGCTAGCTACAACGA GTTGCACA | 11553 |
| 920 | UGCAACGC G UCCGGGCU | 2805 | AGCCCGGA GGCTAGCTACAACGA GCGTTGCA | 11554 |
| 926 | GCGUCCGG G CUGUACCA | 2806 | TGGTACAG GGCTAGCTACAACGA CCGGACGC | 11555 |
| 929 | UCCGGGCU G UACCAUGU | 2807 | ACATGGTA GGCTAGCTACAACGA AGCCCGGA | 11556 |
| 931 | CGGGCUGU A CCAUGUCA | 2808 | TGACATGG GGCTAGCTACAACGA ACAGCCCG | 11557 |
| 934 | GCUGUACC A UGUCACGA | 2809 | TCGTGACA GGCTAGCTACAACGA GGTACAGC | 11558 |
| 936 | UGUACCAU G UCACGAAC | 2810 | GTTCGTGA GGCTAGCTACAACGA ATGGTACA | 11559 |
| 939 | ACCAUGUC A CGAACGAU | 2811 | ATCGTTCG GGCTAGCTACAACGA GACATGGT | 11560 |
| 943 | UGUCACGA A CGAUUGCU | 2812 | AGCAATCG GGCTAGCTACAACGA TCGTGACA | 11561 |
| 946 | CACGAACG A UUGCUCCA | 2813 | TGGAGCAA GGCTAGCTACAACGA CGTTCGTG | 11562 |
| 949 | GAACGAUU G CUCCAACU | 2814 | AGTTGGAG GGCTAGCTACAACGA AATCGTTC | 11563 |
| 955 | UUGCUCCA A CUCAAGCA | 2815 | TGCTTGAG GGCTAGCTACAACGA TGGAGCAA | 11564 |
| 961 | CAACUCAA G CAUUGUGU | 2816 | ACACAATG GGCTAGCTACAACCA TTGAGTTG | 11565 |
| 963 | ACUCAAGC A UUGUGUAU | 2817 | ATACACAA GGCTAGCTACAACGA GCTTGAGT | 11566 |
| 966 | CAAGCAUU G UGUAUGAG | 2818 | CTCATACA GGCTAGCTACAACGA AATGCTTG | 11567 |
| 968 | AGCAUUGU G UAUGAGGC | 2819 | GCCTCATA GGCTAGCTACAACGA ACAATGCT | 11568 |
| 970 | CAUUGUGU A UGAGGCAG | 2820 | CTGCCTCA GGCTAGCTACAACGA ACACAATG | 11569 |
| 975 | UGUAUGAG G CAGAGGAC | 2821 | GTCCTCTG GGCTAGCTACAACGA CTCATACA | 11570 |
| 982 | GGCAGAGG A CAUGAUCA | 2822 | TGATCATG GGCTAGCTACAACGA CCTCTGCC | 11571 |
| 984 | CAGAGGAC A UGAUCAUG | 2823 | CATGATCA GGCTAGCTACAACGA GTCCTCTG | 11572 |
| 987 | AGGACAUG A UCAUGCAC | 2824 | GTGCATGA GGCTAGCTACAACGA CATGTCCT | 11573 |
| 990 | ACAUGAUC A UGCACACC | 2825 | GGTGTGCA GGCTAGCTACAACGA GATCATGT | 11574 |
| 992 | AUGAUCAU G CACACCCC | 2826 | GGGGTGTG GGCTAGCTACAACGA ATGATCAT | 11575 |
| 994 | GAUCAUGC A CACCCCGG | 2827 | CCGGGGTG GGCTAGCTACAACGA GCATGATC | 11576 |
| 996 | UCAUGCAC A CCCCGGGG | 2828 | CCCCGGGG GGCTAGCTACAACGA GTGCATGA | 11577 |
| 1004 | ACCCCGGG G UGCGUGCC | 2829 | GGCACGCA GGCTAGCTACAACGA CCCGGGGT | 11578 |
| 1006 | CCCGGGGU G CGUGCCCU | 2830 | AGGGCACG GGCTAGCTACAACGA ACCCCGGG | 11579 |
| 1008 | CGGGGUGC G UGCCCUGC | 2831 | GCAGGGCA GGCTAGCTACAACGA GCACCCCG | 11580 |
| 1010 | GGGUGCGU G CCCUGCGU | 2832 | ACGCAGGG GGCTAGCTACAACGA ACGCACCC | 11581 |
| 1015 | CGUGCCCU G CGUUCGGG | 2833 | CCCGAACG GGCTAGCTACAACGA AGGGCACG | 11582 |
| 1017 | UGCCCUGC G UUCGGGAG | 2834 | CTCCCGAA GGCTAGCTACAACGA GCAGGGCA | 11583 |
| 1027 | UCGGGAGA A CAACUCCU | 2835 | AGGAGTTG GGCTAGCTACAACGA TCTCCCGA | 11584 |
| 1030 | GGAGAACA A CUCCUCCC | 2836 | GGGAGGAG GGCTAGCTACAACGA TGTTCTCC | 11585 |
| 1039 | CUCCUCCC G CUGCUGGG | 2837 | CCCAGCAG GGCTAGCTACAACGA GGGAGGAG | 11586 |
| 1042 | CUCCCGCU G CUGGGUAG | 2838 | CTACCCAG GGCTAGCTACAACGA AGCGGGAG | 11587 |
| 1047 | GCUGCUGG G UAGCGCUC | 2839 | GAGCGCTA GGCTAGCTACAACGA CCAGCAGC | 11588 |
| 1050 | GCUGGGUA G CGCUCACU | 2840 | AGTGAGCG GGCTAGCTACAACGA TACCCAGC | 11589 |
| 1052 | UGGGUAGC G CUCACUCC | 2841 | GGAGTGAG GGCTAGCTACAACGA GCTACCCA | 11590 |
| 1056 | UAGCGCUC A CUCCCACG | 2842 | CGTGGGAG GGCTAGCTACAACGA GAGCGCTA | 11591 |
| 1062 | UCACUCCC A CGCUCGCG | 2843 | CGCGAGCG GGCTAGCTACAACGA GGGAGTGA | 11592 |
| 1064 | ACUCCCAC G CUCGCGGC | 2844 | GCCGCGAG GGCTAGCTACAACGA GTGGGAGT | 11593 |
| 1068 | CCACGCUC G CGGCCAGG | 2845 | CCTGGCCG GGCTAGCTACAACGA GAGCGTGG | 11594 |
| 1071 | CGCUCGCG G CCAGGAAU | 2846 | ATTCCTGG GGCTAGCTACAACGA CGCGAGCG | 11595 |
| 1078 | GGCCAGGA A UGCCAGCA | 2847 | TGCTGGCA GGCTAGCTACAACGA TCCTGGCC | 11596 |
| 1080 | CCAGGAAU G CCAGCAUC | 2848 | GATGCTGG GGCTAGCTACAACGA ATTCCTGG | 11597 |
| 1084 | GAAUGCCA G CAUCCCCA | 2849 | TGGGGATG GGCTAGCTACAACGA TGGCATTC | 11598 |
| 1086 | AUGCCAGC A UCCCCACU | 2850 | AGTGGGGA GGCTAGCTACAACGA GCTGGCAT | 11599 |
| 1092 | GCAUCCCC A CUACGACG | 2851 | CGTCGTAG GGCTAGCTACAACGA GGGGATGC | 11600 |
| 1095 | UCCCCACU A CGACGAUA | 2852 | TATCGTCG GGCTAGCTACAACGA AGTGGGGA | 11601 |
| 1098 | CCACUACG A CGAUACGG | 2853 | CCGTATCG GGCTAGCTACAACGA CGTAGTGG | 11602 |
| 1101 | CUACGACG A UACGGCGU | 2854 | ACGCCGTA GGCTAGCTACAACGA CGTCGTAG | 11603 |
| 1103 | ACGACGAU A CGGCGUCA | 2855 | TGACGCCG GGCTAGCTACAACGA ATCGTCGT | 11604 |
| 1106 | ACGAUACG G CGUCACGU | 2856 | ACGTGACG GGCTAGCTACAACGA CGTATCGT | 11605 |
| 1108 | GAUACGGC G UCACGUCG | 2857 | CGACGTGA GGCTAGCTACAACGA GCCGTATC | 11606 |
| 1111 | ACGGCGUC A CGUCGAUU | 2858 | AATCGACG GGCTAGCTACAACGA GACGCCGT | 11607 |
| 1113 | GGCGUCAC G UCGAUUUG | 2859 | CAAATCGA GGCTAGCTACAACGA GTGACGCC | 11608 |
| 1117 | UCACGUCG A UUUGCUCG | 2860 | CGAGCAAA GGCTAGCTACAACGA CGACGTGA | 11609 |
| 1121 | GUCGAUUU G CUCGUUGG | 2861 | CCAACGAG GGCTAGCTACAACGA AAATCGAC | 11610 |
| 1125 | AUUUGCUC G UUGGGGCG | 2862 | CGCCCCAA GGCTAGCTACAACGA GAGCAAAT | 11611 |
| 1131 | UCGUUGGG G CGGCUGCU | 2863 | AGCAGCCG GGCTAGCTACAACGA CCCAACGA | 11612 |
| 1134 | UUGGGGCG G CUGCUUUC | 2864 | GAAAGCAG GGCTAGCTACAACGA CGCCCCAA | 11613 |
| 1137 | GGGCGGCU G CUUUCUGC | 2865 | GCAGAAAG GGCTAGCTACAACGA AGCCGCCC | 11614 |
| 1144 | UGCUUUCU G CUCUGCUA | 2866 | TAGCAGAG GGCTAGCTACAACGA AGAAAGCA | 11615 |
| 1149 | UCUGCUCU G CUAUGUAC | 2867 | GTACATAG GGCTAGCTACAACGA AGAGCAGA | 11616 |
| 1152 | GCUCUGCU A UGUACGUG | 2868 | CACGTACA GGCTAGCTACAACGA AGCAGAGC | 11617 |
| 1154 | UCUGCUAU G UACGUGGG | 2869 | CCCACGTA GGCTAGCTACAACGA ATAGCAGA | 11618 |
| 1156 | UGCUAUGU A CGUGGGGG | 2870 | CCCCCACG GGCTAGCTACAACGA ACATAGCA | 11619 |
| 1158 | CUAUGUAC G UGGGGGAU | 2871 | ATCCCCCA GGCTAGCTACAACGA GTACATAG | 11620 |
| 1165 | CGUGGGGG A UCUCUGCG | 2872 | CGCAGAGA GGCTAGCTACAACGA CCCCCACG | 11621 |
| 1171 | GGAUCUCU G CGGAUCUG | 2873 | CAGATCCG GGCTAGCTACAACGA AGAGATCC | 11622 |
| 1175 | CUCUGCGG A UCUGUCUU | 2874 | AAGACAGA GGCTAGCTACAACGA CCGCAGAG | 11623 |
| 1179 | GCGGAUCU G UCUUCCUC | 2875 | GAGGAAGA GGCTAGCTACAACGA AGATCCGC | 11624 |
| 1188 | UCUUCCUC G UCUCUCAG | 2876 | CTGAGAGA GGCTAGCTACAACGA GAGGAAGA | 11625 |
| 1196 | GUCUCUCA G CUGUUCAC | 2877 | GTGAACAG GGCTAGCTACAACGA TGAGAGAC | 11626 |
| 1199 | UCUCAGCU G UUCACCUU | 2878 | AAGGTGAA GGCTAGCTACAACGA AGCTGAGA | 11627 |
| 1203 | AGCUGUUC A CCUUCUCG | 2879 | CGAGAAGG GGCTAGCTACAACGA GAACAGCT | 11628 |
| 1211 | ACCUUCUC G CCUCGCCG | 2880 | CGGCGAGG GGCTAGCTACAACGA GAGAAGGT | 11629 |
| 1216 | CUCGCCUC G CCGGUAUG | 2881 | CATACCGG GGCTAGCTACAACGA GAGGCGAG | 11630 |
| 1220 | CCUCGCCG G UAUGAGAC | 2882 | GTCTCATA GGCTAGCTACAACGA CGGCGAGG | 11631 |
| 1222 | UCGCCGGU A UGAGACAG | 2883 | CTGTCTCA GGCTAGCTACAACGA ACCGGCGA | 11632 |
| 1227 | GGUAUGAG A CAGUACAG | 2884 | CTGTACTG GGCTAGCTACAACGA CTCATACC | 11633 |
| 1230 | AUGAGACA G UACAGGAC | 2885 | GTCCTGTA GGCTAGCTACAACGA TGTCTCAT | 11634 |
| 1232 | GAGACAGU A CAGGACUG | 2886 | CAGTCCTG GGCTAGCTACAACGA ACTGTCTC | 11635 |
| 1237 | AGUACAGG A CUGUAAUU | 2887 | AATTACAG GGCTAGCTACAACGA CCTGTACT | 11636 |
| 1240 | ACAGGACU G UAAUUGCU | 2888 | AGCAATTA GGCTAGCTACAACGA AGTCCTGT | 11637 |
| 1243 | GGACUGUA A UUGCUCGA | 2889 | TCGAGCAA GGCTAGCTACAACGA TACAGTCC | 11638 |
| 1246 | CUGUAAUU G CUCGAUCU | 2890 | AGATCGAG GGCTAGCTACAACGA AATTACAG | 11639 |
| 1251 | AUUGCUCG A UCUAUCCC | 2891 | GGGATAGA GGCTAGCTACAACGA CGAGCAAT | 11640 |
| 1255 | CUCGAUCU A UCCCGGCC | 2892 | GGCCGGGA GGCTAGCTACAACGA AGATCGAG | 11641 |
| 1261 | CUAUCCCG G CCACGUAU | 2893 | ATACGTGG GGCTAGCTACAACGA CGGGATAG | 11642 |
| 1264 | UCCCGGCC A CGUAUCAG | 2894 | CTGATACG GGCTAGCTACAACGA GGCCGGGA | 11643 |
| 1266 | CCGGCCAC G UAUCAGGC | 2895 | GCCTGATA GGCTAGCTACAACGA GTGGCCGG | 11644 |
| 1268 | GGCCACGU A UCAGGCCA | 2896 | TGGCCTGA GGCTAGCTACAACGA ACGTGGCC | 11645 |
| 1273 | CGUAUCAG G CCAUCGCA | 2897 | TGCGATGG GGCTAGCTACAACGA CTGATACG | 11646 |
| 1276 | AUCAGGCC A UCGCAUGG | 2898 | CCATGCGA GGCTAGCTACAACGA GGCCTGAT | 11647 |
| 1279 | AGGCCAUC G CAUGGCUU | 2899 | AAGCCATG GGCTAGCTACAACGA GATGGCCT | 11648 |
| 1281 | GCCAUCGC A UGGCUUGG | 2900 | CCAAGCCA GGCTAGCTACAACGA GCGATGGC | 11649 |
| 1284 | AUCGCAUG G CUUGGGAU | 2901 | ATCCCAAG GGCTAGCTACAACGA CATGCGAT | 11650 |
| 1291 | GGCUUGGG A UAUGAUGA | 2902 | TCATCATA GGCTAGCTACAACGA CCCAAGCC | 11651 |
| 1293 | CUUGGGAU A UGAUGAUG | 2903 | CATCATCA GGCTAGCTACAACGA ATCCCAAG | 11652 |
| 1296 | GGGAUAUG A UGAUGAAU | 2904 | ATTCATCA GGCTAGCTACAACGA CATATCCC | 11653 |
| 1299 | AUAUGAUG A UGAAUUGG | 2905 | CCAATTCA GGCTAGCTACAACGA CATCATAT | 11654 |
| 1303 | GAUGAUGA A UUGGUCAC | 2906 | GTGACCAA GGCTAGCTACAACGA TCATCATC | 11655 |
| 1307 | AUGAAUUG G UCACCUAC | 2907 | GTAGGTGA GGCTAGCTACAACGA CAATTCAT | 11656 |
| 1310 | AAUUGGUC A CCUACAAC | 2908 | GTTGTAGG GGCTAGCTACAACGA GACCAATT | 11657 |
| 1314 | GGUCACCU A CAACAGCC | 2909 | GGCTGTTG GGCTAGCTACAACGA AGGTGACC | 11658 |
| 1317 | CACCUACA A CAGCCCUA | 2910 | TAGGGCTG GGCTAGCTACAACGA TGTAGGTG | 11659 |
| 1320 | CUACAACA G CCCUAGUG | 2911 | CACTAGGG GGCTAGCTACAACGA TGTTGTAG | 11660 |
| 1326 | CAGCCCUA G UGGUAUCG | 2912 | CGATACCA GGCTAGCTACAACGA TAGGGCTG | 11661 |
| 1329 | CCCUAGUG G UAUCGCAG | 2913 | CTGCGATA GGCTAGCTACAACGA CACTAGGG | 11662 |
| 1331 | CUAGUGGU A UCGCAGUU | 2914 | AACTGCGA GGCTAGCTACAACGA ACCACTAG | 11663 |
| 1334 | GUGGUAUC G CAGUUGCU | 2915 | AGCAACTG GGCTAGCTACAACGA GATACCAC | 11664 |
| 1337 | GUAUCGCA G UUGCUCCG | 2916 | CGGAGCAA GGCTAGCTACAACGA TGCGATAC | 11665 |
| 1340 | UCGCAGUU G CUCCGGAU | 2917 | ATCCGGAG GGCTAGCTACAACGA AACTGCGA | 11666 |
| 1347 | UGCUCCGG A UCCCACAA | 2918 | TTGTGGGA GGCTAGCTACAACGA CCGGAGCA | 11667 |
| 1352 | CGGAUCCC A CAAGCCGU | 2919 | ACGGCTTG GGCTAGCTACAACGA GGGATCCG | 11668 |
| 1356 | UCCCACAA G CCGUCGUG | 2920 | CACGACGG GGCTAGCTACAACGA TTGTGGGA | 11669 |
| 1359 | CACAAGCC G UCGUGGAC | 2921 | GTCCACGA GGCTAGCTACAACGA GGCTTGTG | 11670 |
| 1362 | AAGCCGUC G UGGACAUG | 2922 | CATGTCCA GGCTAGCTACAACGA GACGGCTT | 11671 |
| 1366 | CGUCGUGG A CAUGGUGG | 2923 | CCACCATG GGCTAGCTACAACGA CCACGACG | 11672 |
| 1368 | UCGUGGAC A UGGUGGCG | 2924 | CGCCACCA GGCTAGCTACAACGA GTCCACGA | 11673 |
| 1371 | UGGACAUG G UGGCGGGG | 2925 | CCCCGCCA GGCTAGCTACAACGA CATGTCCA | 11674 |
| 1374 | ACAUGGUG G CGGGGGCC | 2926 | GGCCCCCG GGCTAGCTACAACGA CACCATGT | 11675 |
| 1380 | UGGCGGGG G CCCACUGG | 2927 | CCAGTGGG GGCTAGCTACAACGA CCCCGCCA | 11676 |
| 1384 | GGGGGCCC A CUGGGGAG | 2928 | CTCCCCAG GGCTAGCTACAACGA GGGCCCCC | 11677 |
| 1392 | ACUGGGGA G UCCUGGCG | 2929 | CGCCAGGA GGCTAGCTACAACGA TCCCCAGT | 11678 |
| 1398 | GAGUCCUG G CGGGCCUU | 2930 | AAGGCCCG GGCTAGCTACAACGA CAGGACTC | 11679 |
| 1402 | CCUGGCGG G CCUUGCCU | 2931 | AGGCAAGG GGCTAGCTACAACGA CCGCCAGG | 11680 |
| 1407 | CGGGCCUU G CCUAUUAU | 2932 | ATAATAGG GGCTAGCTACAACGA AAGGCCCG | 11681 |
| 1411 | CCUUGCCU A UUAUUCCA | 2933 | TGGAATAA GGCTAGCTACAACGA AGGCAAGG | 11682 |
| 1414 | UGCCUAUU A UUCCAUGG | 2934 | CCATGGAA GGCTAGCTACAACGA AATAGGCA | 11683 |
| 1419 | AUUAUUCC A UGGUGGGG | 2935 | CCCCACCA GGCTAGCTACAACGA GGAATAAT | 11684 |
| 1422 | AUUCCAUG G UGGGGAAC | 2936 | GTTCCCCA GGCTAGCTACAACGA CATGGAAT | 11685 |
| 1429 | GGUGGGGA A CUGGGCUA | 2937 | TAGCCCAG GGCTAGCTACAACGA TCCCCACC | 11686 |
| 1434 | GGAACUGG G CUAAGGUG | 2938 | CACCTTAG GGCTAGCTACAACGA CCAGTTCC | 11687 |
| 1440 | GGGCUAAG G UGUUGAUU | 2939 | AATCAACA GGCTAGCTACAACGA CTTAGCCC | 11688 |
| 1442 | GCUAAGGU G UUGAUUGU | 2940 | ACAATCAA GGCTAGCTACAACGA ACCTTAGC | 11689 |
| 1446 | AGGUGUUG A UUGUGAUG | 2941 | CATCACAA GGCTAGCTACAACGA CAACACCT | 11690 |
| 1449 | UGUUGAUU G UGAUGCUA | 2942 | TAGCATCA GGCTAGCTACAACGA AATCAACA | 11691 |
| 1452 | UGAUUGUG A UGCUACUC | 2943 | GAGTAGCA GGCTAGCTACAACGA CACAATCA | 11692 |
| 1454 | AUUGUGAU G CUACUCUU | 2944 | AAGAGTAG GGCTAGCTACAACGA ATCACAAT | 11693 |
| 1457 | GUGAUGCU A CUCUUUGC | 2945 | GCAAAGAG GGCTAGCTACAACGA AGCATCAC | 11694 |
| 1464 | UACUCUUU G CCGGCGUU | 2946 | AACGCCGG GGCTAGCTACAACGA AAAGAGTA | 11695 |
| 1468 | CUUUGCCG G CGUUGACG | 2947 | CGTCAACG GGCTAGCTACAACGA CGGCAAAG | 11696 |
| 1470 | UUGCCGGC G UUGACGGG | 2948 | CCCGTCAA GGCTAGCTACAACGA GCCGGCAA | 11697 |
| 1474 | CGGCGUUG A CGGGGACA | 2949 | TGTCCCCG GGCTAGCTACAACGA CAACGCCG | 11698 |
| 1480 | UGACGGGG A CACCUACA | 2950 | TGTAGGTG GGCTAGCTACAACGA CCCCGTCA | 11699 |
| 1482 | ACGGGGAC A CCUACACG | 2951 | CGTGTAGG GGCTAGCTACAACGA GTCCCCGT | 11700 |
| 1486 | GGACACCU A CACGACAG | 2952 | CTGTCGTG GGCTAGCTACAACGA AGGTGTCC | 11701 |
| 1488 | ACACCUAC A CGACAGGG | 2953 | CCCTGTCG GGCTAGCTACAACGA GTAGGTGT | 11702 |
| 1491 | CCUACACG A CAGGGGGG | 2954 | CCCCCCTG GGCTAGCTACAACGA CGTGTAGG | 11703 |
| 1500 | CAGGGGGG G CGCAGGGC | 2955 | GCCCTGCG GGCTAGCTACAACGA CCCCCCTG | 11704 |
| 1502 | GGGGGGGC G CAGGGCCA | 2956 | TGGCCCTG GGCTAGCTACAACGA GCCCCCCC | 11705 |
| 1507 | GGCGCAGG G CCACACCA | 2957 | TGGTGTGG GGCTAGCTACAACGA CCTGCGCC | 11706 |
| 1510 | GCAGGGCC A CACCACUA | 2958 | TAGTGGTG GGCTAGCTACAACGA GGCCCTGC | 11707 |
| 1512 | AGGGCCAC A CCACUAGU | 2959 | ACTAGTGG GGCTAGCTACAACGA GTGGCCCT | 11708 |
| 1515 | GCCACACC A CUAGUAGG | 2960 | CCTACTAG GGCTAGCTACAACGA GGTGTGGC | 11709 |
| 1519 | CACCACUA G UAGGGUGG | 2961 | CCACCCTA GGCTAGCTACAACGA TAGTGGTG | 11710 |
| 1524 | CUAGUAGG G UGGCAUCC | 2962 | GGATGCCA GGCTAGCTACAACGA CCTACTAG | 11711 |
| 1527 | GUAGGGUG G CAUCCCUC | 2963 | GAGGGATG GGCTAGCTACAACGA CACCCTAC | 11712 |
| 1529 | AGGGUGGC A UCCCUCUU | 2964 | AAGAGGGA GGCTAGCTACAACGA GCCACCCT | 11713 |
| 1539 | CCCUCUUU A CAUCUGGA | 2965 | TCCAGATG GGCTAGCTACAACGA AAAGAGGG | 11714 |
| 1541 | CUCUUUAC A UCUGGAGC | 2966 | GCTCCAGA GGCTAGCTACAACGA GTAAAGAG | 11715 |
| 1548 | CAUCUGGA G CAUCUCAG | 2967 | CTGAGATG GGCTAGCTACAACGA TCCAGATG | 11716 |
| 1550 | UCUGGAGC A UCUCAGAA | 2968 | TTCTGAGA GGCTAGCTACAACGA GCTCCAGA | 11717 |
| 1558 | AUCUCAGA A UAUCCAGC | 2969 | GCTGGATA GGCTAGCTACAACGA TCTGAGAT | 11718 |
| 1560 | CUCAGAAU A UCCAGCUU | 2970 | AAGCTGGA GGCTAGCTACAACGA ATTCTGAG | 11719 |
| 1565 | AAUAUCCA G CUUAUUAA | 2971 | TTAATAAG GGCTAGCTACAACGA TGGATATT | 11720 |
| 1569 | UCCAGCUU A UUAACACC | 2972 | GGTGTTAA GGCTAGCTACAACGA AAGCTGGA | 11721 |
| 1573 | GCUUAUUA A CACCAACG | 2973 | CGTTGGTG GGCTACCTACAACGA TAATAAGC | 11722 |
| 1575 | UUAUUAAC A CCAACGGC | 2974 | GCCGTTGG GGCTAGCTACAACGA GTTAATAA | 11723 |
| 1579 | UAACACCA A CGGCAGCU | 2975 | AGCTGCCG GGCTAGCTACAACGA TGGTGTTA | 11724 |
| 1582 | CACCAACG G CAGCUGGC | 2976 | GCCAGCTG GGCTAGCTACAACGA CGTTGGTG | 11725 |
| 1585 | CAACGGCA G CUGGCACA | 2977 | TGTGCCAG GGCTAGCTACAACGA TGCCGTTG | 11726 |
| 1589 | GGCAGCUG G CACAUUAA | 2978 | TTAATGTG GGCTAGCTACAACGA CAGCTGCC | 11727 |
| 1591 | CAGCUGGC A CAUUAACA | 2979 | TGTTAATG GGCTAGCTACAACGA GCCAGCTG | 11728 |
| 1593 | GCUGGCAC A UUAACAGG | 2980 | CCTGTTAA GGCTAGCTACAACGA GTGCCAGC | 11729 |
| 1597 | GCACAUUA A CAGGACUG | 2981 | CAGTCCTG GGCTAGCTACAACGA TAATGTGC | 11730 |
| 1602 | UUAACAGG A CUGCCCUG | 2982 | CAGGGCAG GGCTAGCTACAACGA CCTGTTAA | 11731 |
| 1605 | ACAGGACU G CCCUGAAC | 2983 | GTTCAGGG GGCTAGCTACAACGA AGTCCTGT | 11732 |
| 1612 | UGCCCUGA A CUGCAAUG | 2984 | CATTGCAG GGCTAGCTACAACGA TCAGGGCA | 11733 |
| 1615 | CCUGAACU G CAAUGACU | 2985 | AGTCATTG GGCTAGCTACAACGA AGTTCAGG | 11734 |
| 1618 | GAACUGCA A UGACUCCC | 2986 | GGGAGTCA GGCTAGCTACAACGA TGCAGTTC | 11735 |
| 1621 | CUGCAAUG A CUCCCUCC | 2987 | GGAGGGAG GGCTAGCTACAACGA CATTGCAG | 11736 |
| 1632 | CCCUCCAA A CCGGGUUC | 2988 | GAACCCGG GGCTAGCTACAACGA TTGGAGGG | 11737 |
| 1637 | CAAACCGG G UUCAUUGC | 2989 | GCAATGAA GGCTAGCTACAACGA CCGGTTTG | 11738 |
| 1641 | CCGGGUUC A UUGCUGCA | 2990 | TGCAGCAA GGCTAGCTACAACGA GAACCCGG | 11739 |
| 1644 | GGUUCAUU G CUGCACUG | 2991 | CAGTGCAG GGCTAGCTACAACGA AATGAACC | 11740 |
| 1647 | UCAUUGCU G CACUGUUC | 2992 | GAACAGTG GGCTAGCTACAACGA AGCAATGA | 11741 |
| 1649 | AUUGCUGC A CUGUUCUA | 2993 | TAGAACAG GGCTAGCTACAACGA GCAGCAAT | 11742 |
| 1652 | GCUGCACU G UUCUAUGC | 2994 | GCATAGAA GGCTAGCTACAACGA AGTGCAGC | 11743 |
| 1657 | ACUGUUCU A UGCACACA | 2995 | TGTGTGCA GGCTAGCTACAACGA AGAACAGT | 11744 |
| 1659 | UGUUCUAU G CACACAGG | 2996 | CCTGTGTG GGCTAGCTACAACGA ATAGAACA | 11745 |
| 1661 | UUCUAUGC A CACAGGUU | 2997 | AACCTGTG GGCTAGCTACAACGA GCATAGAA | 11746 |
| 1663 | CUAUGCAC A CAGGUUCA | 2998 | TGAACCTG GGCTAGCTACAACGA GTGCATAG | 11747 |
| 1667 | GCACACAG G UUCAACUC | 2999 | GAGTTGAA GGCTAGCTACAACGA CTGTGTGC | 11748 |
| 1672 | CAGGUUCA A CUCGUCCG | 3000 | CGGACGAG GGCTAGCTACAACGA TGAACCTG | 11749 |
| 1676 | UUCAACUC G UCCGGAUG | 3001 | CATCCGGA GGCTAGCTACAACGA GAGTTGAA | 11750 |
| 1682 | UCGUCCGG A UGCCCACA | 3002 | TGTGGGCA GGCTAGCTACAACGA CCGGACGA | 11751 |
| 1684 | GUCCGGAU G CCCACAGC | 3003 | GCTGTGGG GGCTAGCTACAACGA ATCCGGAC | 11752 |
| 1688 | GGAUGCCC A CAGCGCUU | 3004 | AAGCGCTG GGCTAGCTACAACGA GGGCATCC | 11753 |
| 1691 | UGCCCACA G CGCUUGGC | 3005 | GCCAAGCG GGCTAGCTACAACGA TGTGGGCA | 11754 |
| 1693 | CCCACAGC G CUUGGCCA | 3006 | TGGCCAAG GGCTAGCTACAACGA GCTGTGGG | 11755 |
| 1698 | AGCGCUUG G CCAGCUGC | 3007 | GCAGCTGG GGCTAGCTACAACGA CAAGCGCT | 11756 |
| 1702 | CUUGGCCA G CUGCCGCU | 3008 | AGCGGCAG GGCTAGCTACAACGA TGGCCAAG | 11757 |
| 1705 | GGCCAGCU G CCGCUCCA | 3009 | TGGAGCGG GGCTAGCTACAACGA AGCTGGCC | 11758 |
| 1708 | CAGCUGCC G CUCCAUUG | 3010 | CAATGGAG GGCTAGCTACAACGA GGCAGCTG | 11759 |
| 1713 | GCCGCUCC A UUGACAAG | 3011 | CTTGTCAA GGCTAGCTACAACGA GGAGCGGC | 11760 |
| 1717 | CUCCAUUG A CAAGUUCG | 3012 | CGAACTTG GGCTAGCTACAACGA CAATGGAG | 11761 |
| 1721 | AUUGACAA G UUCGCUCA | 3013 | TGAGCGAA GGCTAGCTACAACGA TTGTCAAT | 11762 |
| 1725 | ACAAGUUC G CUCAGGGG | 3014 | CCCCTGAG GGCTAGCTACAACGA GAACTTGT | 11763 |
| 1733 | GCUCAGGG G UGGGGUCC | 3015 | GGACCCCA GGCTAGCTACAACGA CCCTGAGC | 11764 |
| 1738 | GGGGUGGG G UCCUAUCA | 3016 | TGATAGGA GGCTAGCTACAACGA CCCACCCC | 11765 |
| 1743 | GGGGUCCU A UCACCUAC | 3017 | GTAGGTGA GGCTAGCTACAACGA AGGACCCC | 11766 |
| 1746 | GUCCUAUC A CCUACACC | 3018 | GGTGTAGG GGCTAGCTACAACGA GATAGGAC | 11767 |
| 1750 | UAUCACCU A CACCGAGG | 3019 | CCTCGGTG GGCTAGCTACAACGA AGGTGATA | 11768 |
| 1752 | UCACCUAC A CCGAGGGC | 3020 | GCCCTCGG GGCTAGCTACAACGA GTAGGTGA | 11769 |
| 1759 | CACCGAGG G CCACAACU | 3021 | AGTTGTGG GGCTAGCTACAACGA CCTCGGTG | 11770 |
| 1762 | CGAGGGCC A CAACUCGG | 3022 | CCGAGTTG GGCTAGCTACAACGA GGCCCTCG | 11771 |
| 1765 | GGGCCACA A CUCGGACC | 3023 | GGTCCGAG GGCTAGCTACAACGA TGTGGCCC | 11772 |
| 1771 | CAACUCGG A CCAGAGGC | 3024 | GCCTCTGG GGCTAGCTACAACGA CCGAGTTG | 11773 |
| 1778 | GACCAGAG G CCCUAUUG | 3025 | CAATAGGG GGCTAGCTACAACGA CTCTGGTC | 11774 |
| 1783 | GAGGCCCU A UUGCUGGC | 3026 | GCCAGCAA GGCTAGCTACAACGA AGGGCCTC | 11775 |
| 1786 | GCCCUAUU G CUGGCACU | 3027 | AGTGCCAG GGCTAGCTACAACGA AATAGGGC | 11776 |
| 1790 | UAUUGCUG G CACUACGC | 3028 | GCGTAGTG GGCTAGCTACAACGA CAGCAATA | 11777 |
| 1792 | UUGCUGGC A CUACGCAC | 3029 | GTGCGTAG GGCTAGCTACAACGA GCCAGCAA | 11778 |
| 1795 | CUGGCACU A CGCACCGC | 3030 | GCGGTGCG GGCTAGCTACAACGA AGTGCCAG | 11779 |
| 1797 | GGCACUAC G CACCGCGG | 3031 | CCGCGGTG GGCTAGCTACAACGA GTAGTGCC | 11780 |
| 1799 | CACUACGC A CCGCGGCC | 3032 | GGCCGCGG GGCTAGCTACAACGA GCGTAGTG | 11781 |
| 1802 | UACGCACC G CGGCCGUG | 3033 | CACGGCCG GGCTAGCTACAACGA GGTGCGTA | 11782 |
| 1805 | GCACCGCG G CCGUGUGG | 3034 | CCACACGG GGCTAGCTACAACGA CGCGGTGC | 11783 |
| 1808 | CCGCGGCC G UGUGGUAU | 3035 | ATACCACA GGCTAGCTACAACGA GGCCGCGG | 11784 |
| 1810 | GCGGCCGU G UGGUAUCG | 3036 | CGATACCA GGCTAGCTACAACGA ACGGCCGC | 11785 |
| 1813 | GCCGUGUG G UAUCGUAC | 3037 | GTACGATA GGCTAGCTACAACGA CACACGGC | 11786 |
| 1815 | CGUGUGGU A UCGUACCC | 3038 | GGGTACGA GGCTAGCTACAACGA ACCACACG | 11787 |
| 1818 | GUGGUAUC G UACCCGCA | 3039 | TGCGGGTA GGCTAGCTACAACGA GATACCAC | 11788 |
| 1820 | GGUAUCGU A CCCGCAUC | 3040 | GATGCGGG GGCTAGCTACAACGA ACGATACC | 11789 |
| 1824 | UCGUACCC G CAUCGCAG | 3041 | CTGCGATG GGCTAGCTACAACGA GGGTACGA | 11790 |
| 1826 | GUACCCGC A UCGCAGGU | 3042 | ACCTGCGA GGCTAGCTACAACGA GCGGGTAC | 11791 |
| 1829 | CCCGCAUC G CAGGUAUG | 3043 | CATACCTG GGCTAGCTACAACGA GATGCGGG | 11792 |
| 1833 | CAUCGCAG G UAUGUGGU | 3044 | ACCACATA GGCTAGCTACAACGA CTGCGATG | 11793 |
| 1835 | UCGCAGGU A UGUGGUCC | 3045 | GGACCACA GGCTAGCTACAACGA ACCTGCGA | 11794 |
| 1837 | GCAGGUAU G UGGUCCAG | 3046 | CTGGACCA GGCTAGCTACAACGA ATACCTGC | 11795 |
| 1840 | GGUAUGUG G UCCAGUGU | 3047 | ACACTGGA GGCTAGCTACAACGA CACATACC | 11796 |
| 1845 | GUGGUCCA G UGUAUUGC | 3048 | GCAATACA GGCTAGCTACAACGA TGGACCAC | 11797 |
| 1847 | GGUCCAGU G UAUUGCUU | 3049 | AAGCAATA GGCTAGCTACAACGA ACTGGACC | 11798 |
| 1849 | UCCAGUGU A UUGCUUCA | 3050 | TGAAGCAA GGCTAGCTACAACGA ACACTGGA | 11799 |
| 1852 | AGUGUAUU G CUUCACCC | 3051 | GGGTGAAG GGCTAGCTACAACGA AATACACT | 11800 |
| 1857 | AUUGCUUC A CCCCAAGC | 3052 | GCTTGGGG GGCTAGCTACAACGA GAAGCAAT | 11801 |
| 1864 | CACCCCAA G CCCUGUUG | 3053 | CAACAGGG GGCTAGCTACAACGA TTGGGGTG | 11802 |
| 1869 | CAAGCCCU G UUGUGGUG | 3054 | CACCACAA GGCTAGCTACAACGA AGGGCTTG | 11803 |
| 1872 | GCCCUGUU G UGGUGGGG | 3055 | CCCCACCA GGCTAGCTACAACGA AACAGGGC | 11804 |
| 1875 | CUGUUGUG G UGGGGACG | 3056 | CGTCCCCA GGCTAGCTACAACGA CACAACAG | 11805 |
| 1881 | UGGUGGGG A CGACCGAC | 3057 | GTCGGTCG GGCTAGCTACAACGA CCCCACCA | 11806 |
| 1884 | UGGGGACG A CCGACCGU | 3058 | ACGGTCGG GGCTAGCTACAACGA CGTCCCCA | 11807 |
| 1888 | GACGACCG A CCGUUUCG | 3059 | CGAAACGG GGCTAGCTACAACGA CGGTCGTC | 11808 |
| 1891 | GACCGACC G UUUCGGCG | 3060 | CGCCGAAA GGCTAGCTACAACGA GGTCGGTC | 11809 |
| 1897 | CCGUUUCG G CGCCCCCA | 3061 | TGGGGGCG GGCTAGCTACAACGA CGAAACGG | 11810 |
| 1899 | GUUUCGGC G CCCCCACG | 3062 | CGTGGGGG GGCTAGCTACAACGA GCCGAAAC | 11811 |
| 1905 | GCGCCCCC A CGUAUAAC | 3063 | GTTATACG GGCTAGCTACAACGA GGGGGCGC | 11812 |
| 1907 | GCCCCCAC G UAUAACUG | 3064 | CAGTTATA GGCTAGCTACAACGA GTGGGGGC | 11813 |
| 1909 | CCCCACGU A UAACUGGG | 3065 | CCCAGTTA GGCTAGCTACAACGA ACGTGGGG | 11814 |
| 1912 | CACGUAUA A CUGGGGGG | 3066 | CCCCCCAG GGCTAGCTACAACGA TATACGTG | 11815 |
| 1920 | ACUGGGGG G CGAACGAG | 3067 | CTCGTTCG GGCTAGCTACAACGA CCCCCAGT | 11816 |
| 1924 | GGGGGCGA A CGAGACGG | 3068 | CCGTCTCG GGCTAGCTACAACGA TCGCCCCC | 11817 |
| 1929 | CGAACGAG A CGGACGUG | 3069 | CACGTCCG GGCTAGCTACAACGA CTCGTTCG | 11818 |
| 1933 | CGAGACGG A CGUGCUGC | 3070 | GCAGCACG GGCTAGCTACAACGA CCGTCTCG | 11819 |
| 1935 | AGACGGAC G UGCUGCUC | 3071 | GAGCAGCA GGCTAGCTACAACGA GTCCGTCT | 11820 |
| 1937 | ACGGACGU G CUGCUCCU | 3072 | AGGAGCAG GGCTAGCTACAACGA ACGTCCGT | 11821 |
| 1940 | GACGUGCU G CUCCUCAA | 3073 | TTGAGGAG GGCTAGCTACAACGA AGCACGTC | 11822 |
| 1948 | GCUCCUCA A CAACACGC | 3074 | GCGTGTTG GGCTAGCTACAACGA TGAGGAGC | 11823 |
| 1951 | CCUCAACA A CACGCGGC | 3075 | GCCGCGTG GGCTAGCTACAACGA TGTTGAGG | 11824 |
| 1953 | UCAACAAC A CGCGGCCG | 3076 | CGGCCGCG GGCTAGCTACAACGA GTTGTTGA | 11825 |
| 1955 | AACAACAC G CGGCCGCC | 3077 | GGCGGCCG GGCTAGCTACAACGA GTGTTGTT | 11826 |
| 1958 | AACACGCG G CCGCCGCA | 3078 | TGCGGCGG GGCTAGCTACAACGA CGCGTGTT | 11827 |
| 1961 | ACGCGGCC G CCGCAAGG | 3079 | CCTTGCGG GGCTAGCTACAACGA GGCCGCGT | 11828 |
| 1964 | CGGCCGCC G CAAGGCAA | 3080 | TTGCCTTG GGCTAGCTACAACGA GGCGGCCG | 11829 |
| 1969 | GCCGCAAG G CAACUGGU | 3081 | ACCAGTTG GGCTAGCTACAACGA CTTGCGGC | 11830 |
| 1972 | GCAAGGCA A CUGGUUCG | 3082 | CGAACCAG GGCTAGCTACAACGA TGCCTTGC | 11831 |
| 1976 | GGCAACUG G UUCGGCUG | 3083 | CAGCCGAA GGCTAGCTACAACGA CAGTTGCC | 11832 |
| 1981 | CUGGUUCG G CUGCACAU | 3084 | ATGTGCAG GGCTAGCTACAACGA CGAACCAG | 11833 |
| 1984 | GUUCGGCU G CACAUGGA | 3085 | TCCATGTG GGCTAGCTACAACGA AGCCGAAC | 11834 |
| 1986 | UCGGCUGC A CAUGGAUG | 3086 | CATCCATG GGCTAGCTACAACGA GCAGCCGA | 11835 |
| 1988 | GGCUGCAC A UGGAUGAA | 3087 | TTCATCCA GGCTAGCTACAACGA GTGCAGCC | 11836 |
| 1992 | GCACAUGG A UGAAUGGC | 3088 | GCCATTCA GGCTAGCTACAACGA CCATGTGC | 11837 |
| 1996 | AUGGAUGA A UGGCACUG | 3089 | CAGTGCCA GGCTAGCTACAACGA TCATCCAT | 11838 |
| 1999 | GAUGAAUG G CACUGGGU | 3090 | ACCCAGTG GGCTAGCTACAACGA CATTCATC | 11839 |
| 2001 | UGAAUGGC A CUGGGUUC | 3091 | GAACCCAG GGCTAGCTACAACGA GCCATTCA | 11840 |
| 2006 | GGCACUGG G UUCACCAA | 3092 | TTGGTGAA GGCTAGCTACAACGA CCAGTGCC | 11841 |
| 2010 | CUGGGUUC A CCAAGACG | 3093 | CGTCTTGG GGCTAGCTACAACGA GAACCCAG | 11842 |
| 2016 | UCACCAAG A CGUGCGGG | 3094 | CCCGCACG GGCTAGCTACAACGA CTTGGTGA | 11843 |
| 2018 | ACCAAGAC G UGCGGGGG | 3095 | CCCCCGCA GGCTAGCTACAACGA GTCTTGGT | 11844 |
| 2020 | CAAGACGU G CGGGGGCC | 3096 | GGCCCCCG GGCTAGCTACAACGA ACGTCTTG | 11845 |
| 2026 | GUGCGGGG G CCCCCCGU | 3097 | ACGGGGGG GGCTAGCTACAACGA CCCCGCAC | 11846 |
| 2033 | GGCCCCCC G UGCAACAU | 3098 | ATGTTGCA GGCTAGCTACAACGA GGGGGGCC | 11847 |
| 2035 | CCCCCCGU G CAACAUCG | 3099 | CGATGTTG GGCTAGCTACAACGA ACGGGGGG | 11848 |
| 2038 | CCCGUGCA A CAUCGGGG | 3100 | CCCCGATG GGCTAGCTACAACGA TGCACGGG | 11849 |
| 2040 | CGUGCAAC A UCGGGGGG | 3101 | CCCCCCGA GGCTAGCTACAACGA GTTGCACG | 11850 |
| 2049 | UCGGGGGG G CCGGUAAC | 3102 | GTTACCGG GGCTAGCTACAACGA CCCCCCGA | 11851 |
| 2053 | GGGGGCCG G UAACGACA | 3103 | TGTCGTTA GGCTAGCTACAACGA CGGCCCCC | 11852 |
| 2056 | GGCCGGUA A CGACACCU | 3104 | AGGTGTCG GGCTAGCTACAACGA TACCGGCC | 11853 |
| 2059 | CGGUAACG A CACCUUAA | 3105 | TTAAGGTG GGCTAGCTACAACGA CGTTACCG | 11854 |
| 2061 | GUAACGAC A CCUUAACC | 3106 | GGTTAAGG GGCTAGCTACAACGA GTCGTTAC | 11855 |
| 2067 | ACACCUUA A CCUGCCCC | 3107 | GGGGCAGG GGCTAGCTACAACGA TAAGGTGT | 11856 |
| 2071 | CUUAACCU G CCCCACGG | 3108 | CCGTGGGG GGCTAGCTACAACGA AGGTTAAG | 11857 |
| 2076 | CCUGCCCC A CGGACUGC | 3109 | GCAGTCCG GGCTAGCTACAACGA GGGGCAGG | 11858 |
| 2080 | CCCCACGG A CUGCUUCC | 3110 | GGAAGCAG GGCTAGCTACAACGA CCGTGGGG | 11859 |
| 2083 | CACGGACU G CUUCCGGA | 3111 | TCCGGAAG GGCTAGCTACAACGA AGTCCGTG | 11860 |
| 2093 | UUCCGGAA G CACCCCGA | 3112 | TCGGGGTG GGCTAGCTACAACGA TTCCGGAA | 11861 |
| 2095 | CCGGAAGC A CCCCGAGG | 3113 | CCTCGGGG GGCTAGCTACAACGA GCTTCCGG | 11862 |
| 2103 | ACCCCGAG G CCACUUAC | 3114 | GTAAGTGG GGCTAGCTACAACGA CTCGGGGT | 11863 |
| 2106 | CCGAGGCC A CUUACGCA | 3115 | TGCGTAAG GGCTAGCTACAACGA GGCCTCGG | 11864 |
| 2110 | GGCCACUU A CGCAAAGU | 3116 | ACTTTGCG GGCTAGCTACAACGA AAGTGGCC | 11865 |
| 2112 | CCACUUAC G CAAAGUGC | 3117 | GCACTTTG GGCTAGCTACAACGA GTAAGTGG | 11866 |
| 2117 | UACGCAAA G UGCGGUUC | 3118 | GAACCGCA GGCTAGCTACAACGA TTTGCGTA | 11867 |
| 2119 | CGCAAAGU G CGGUUCGG | 3119 | CCGAACCG GGCTAGCTACAACGA ACTTTGCG | 11868 |
| 2122 | AAAGUGCG G UUCGGCGC | 3120 | GCCCCGAA GGCTAGCTACAACGA CGCACTTT | 11869 |
| 2129 | GGUUCGGC G CCUUGGUU | 3121 | AACCAAGG GGCTAGCTACAACGA CCCGAACC | 11870 |
| 2135 | GGGCCUUG G UUAACACC | 3122 | GGTGTTAA GGCTAGCTACAACGA CAAGGCCC | 11871 |
| 2139 | CUUGGUUA A CACCUAGA | 3123 | TCTAGGTG GGCTAGCTACAACGA TAACCAAG | 11872 |
| 2141 | UGGUUAAC A CCUAGAUG | 3124 | CATCTAGG GGCTAGCTACAACGA GTTAACCA | 11873 |
| 2147 | ACACCUAG A UGCAUAGU | 3125 | ACTATGCA GGCTAGCTACAACGA CTAGGTGT | 11874 |
| 2149 | ACCUAGAU G CAUAGUUG | 3126 | CAACTATG GGCTAGCTACAACGA ATCTAGGT | 11875 |
| 2151 | CUAGAUGC A UAGUUGAC | 3127 | GTCAACTA GGCTAGCTACAACGA GCATCTAG | 11876 |
| 2154 | GAUGCAUA G UUGACUAC | 3128 | GTAGTCAA GGCTAGCTACAACGA TATGCATC | 11877 |
| 2158 | CAUAGUUG A CUACCCAU | 3129 | ATGGGTAG GGCTAGCTACAACGA CAACTATG | 11878 |
| 2161 | AGUUGACU A CCCAUACA | 3130 | TGTATGGG GGCTAGCTACAACGA AGTCAACT | 11879 |
| 2165 | GACUACCC A UACAGGCU | 3131 | AGCCTGTA GGCTAGCTACAACGA GGGTAGTC | 11880 |
| 2167 | CUACCCAU A CAGGCUUU | 3132 | AAAGCCTG GGCTAGCTACAACGA ATGGGTAG | 11881 |
| 2171 | CCAUACAG G CUUUGGCA | 3133 | TGCCAAAG GGCTAGCTACAACGA CTGTATGG | 11882 |
| 2177 | AGGCUUUG G CACUACCC | 3134 | GGGTAGTG GGCTAGCTACAACGA CAAAGCCT | 11883 |
| 2179 | GCUUUGGC A CUACCCCU | 3135 | AGGGGTAG GGCTAGCTACAACGA GCCAAAGC | 11884 |
| 2182 | UUGGCACU A CCCCUGCA | 3136 | TGCAGGGG GGCTAGCTACAACGA AGTGCCAA | 11885 |
| 2188 | CUACCCCU G CACUGUCA | 3137 | TGACAGTG GGCTAGCTACAACGA AGGGGTAG | 11886 |
| 2190 | ACCCCUGC A CUGUCAAU | 3138 | ATTGACAG GGCTAGCTACAACGA GCAGGGGT | 11887 |
| 2193 | CCUGCACU G UCAAUUUU | 3139 | AAAATTGA GGCTAGCTACAACGA AGTGCAGG | 11888 |
| 2197 | CACUGUCA A UUUUUCCA | 3140 | TGGAAAAA GGCTAGCTACAACGA TGACAGTG | 11889 |
| 2205 | AUUUUUCC A UCUUUAAG | 3141 | CTTAAAGA GGCTAGCTACAACGA GGAAAAAT | 11890 |
| 2214 | UCUUUAAG G UUAGGAUG | 3142 | CATCCTAA GGCTAGCTACAACGA CTTAAAGA | 11891 |
| 2220 | AGGUUAGG A UGUAUGUG | 3143 | CACATACA GGCTAGCTACAACGA CCTAACCT | 11892 |
| 2222 | GUUAGGAU G UAUGUGGG | 3144 | CCCACATA GGCTAGCTACAACGA ATCCTAAC | 11893 |
| 2224 | UAGGAUGU A UGUGGGGG | 3145 | CCCCCACA GGCTAGCTACAACGA ACATCCTA | 11894 |
| 2226 | GGAUGUAU G UGGGGGGC | 3146 | GCCCCCCA GGCTAGCTACAACGA ATACATCC | 11895 |
| 2233 | UGUGGGGG G CGUGGAGC | 3147 | GCTCCACG GGCTAGCTACAACGA CCCCCACA | 11896 |
| 2235 | UGGGGGGC G UGGAGCAC | 3148 | GTGCTCCA GGCTAGCTACAACGA GCCCCCCA | 11897 |
| 2240 | GGCGUGGA G CACAGGCU | 3149 | AGCCTGTG GGCTAGCTACAACGA TCCACGCC | 11898 |
| 2242 | CGUGGAGC A CAGGCUCA | 3150 | TGAGCCTG GGCTAGCTACAACGA GCTCCACG | 11899 |
| 2246 | GAGCACAG G CUCACCGC | 3151 | GCGGTGAG GGCTAGCTACAACGA CTGTGCTC | 11900 |
| 2250 | ACAGGCUC A CCGCCGCA | 3152 | TGCGGCGG GGCTAGCTACAACGA GAGCCTGT | 11901 |
| 2253 | GGCUCACC G CCGCAUGC | 3153 | GCATGCGG GGCTAGCTACAACGA GGTGAGCC | 11902 |
| 2256 | UCACCGCC G CAUGCAAU | 3154 | ATTGCATG GGCTAGCTACAACGA GGCGGTGA | 11903 |
| 2258 | ACCGCCGC A UGCAAUUG | 3155 | CAATTGCA GGCTAGCTACAACGA GCGGCGGT | 11904 |
| 2260 | CGCCGCAU G CAAUUGGA | 3156 | TCCAATTG GGCTAGCTACAACGA ATGCGGCG | 11905 |
| 2263 | CGCAUGCA A UUGGACUC | 3157 | GAGTCCAA GGCTAGCTACAACGA TGCATGCG | 11906 |
| 2268 | GCAAUUGG A CUCGAGGA | 3158 | TCCTCGAG GGCTAGCTACAACGA CCAATTGC | 11907 |
| 2279 | CGAGGAGA G CGUUGUGA | 3159 | TCACAACG GGCTAGCTACAACGA TCTCCTCG | 11908 |
| 2281 | AGGAGAGC G UUGUGAUU | 3160 | AATCACAA GGCTAGCTACAACGA GCTCTCCT | 11909 |
| 2284 | AGAGCGUU G UGAUUUGG | 3161 | CCAAATCA GGCTAGCTACAACGA AACGCTCT | 11910 |
| 2287 | GCGUUGUG A UUUGGAGG | 3162 | CCTCCAAA GGCTAGCTACAACGA CACAACGC | 11911 |
| 2296 | UUUGGAGG A CAGGGACA | 3163 | TGTCCCTG GGCTAGCTACAACGA CCTCCAAA | 11912 |
| 2302 | GGACAGGG A CAGAUCAG | 3164 | CTGATCTG GGCTAGCTACAACGA CCCTGTCC | 11913 |
| 2306 | AGGGACAG A UCAGAGCU | 3165 | AGCTCTGA GGCTAGCTACAACGA CTGTCCCT | 11914 |
| 2312 | AGAUCAGA G CUCAGCCC | 3166 | GGGCTGAG GGCTAGCTACAACGA TCTGATCT | 11915 |
| 2317 | AGAGCUCA G CCCGCUGC | 3167 | GCAGCGGG GGCTAGCTACAACGA TGAGCTCT | 11916 |
| 2321 | CUCAGCCC G CUGCUGUU | 3168 | AACAGCAG GGCTAGCTACAACGA GGGCTGAG | 11917 |
| 2324 | AGCCCGCU G CUGUUGUC | 3169 | GACAACAG GGCTAGCTACAACGA AGCGGGCT | 11918 |
| 2327 | CCGCUGCU G UUGUCCAC | 3170 | GTGGACAA GGCTAGCTACAACGA AGCAGCGG | 11919 |
| 2330 | CUGCUGUU G UCCACUAC | 3171 | GTAGTGGA GGCTAGCTACAACGA AACAGCAG | 11920 |
| 2334 | UGUUGUCC A CUACAGAG | 3172 | CTCTGTAG GGCTAGCTACAACGA GGACAACA | 11921 |
| 2337 | UGUCCACU A CAGAGUGG | 3173 | CCACTCTG GGCTAGCTACAACGA AGTGGACA | 11922 |
| 2342 | ACUACAGA G UGGCAAAU | 3174 | ATTTGCCA GGCTAGCTACAACGA TCTGTAGT | 11923 |
| 2345 | ACAGAGUG G CAAAUACU | 3175 | AGTATTTG GGCTAGCTACAACGA CACTCTGT | 11924 |
| 2349 | AGUGGCAA A UACUGCCC | 3176 | GGGCAGTA GGCTAGCTACAACGA TTGCCACT | 11925 |
| 2351 | UGGCAAAU A CUGCCCUG | 3177 | CAGGGCAG GGCTAGCTACAACGA ATTTGCCA | 11926 |
| 2354 | CAAAUACU G CCCUGCUC | 3178 | GAGCAGGG GGCTAGCTACAACGA AGTATTTG | 11927 |
| 2359 | ACUGCCCU G CUCCUUCA | 3179 | TGAAGGAG GGCTAGCTACAACGA AGGGCAGT | 11928 |
| 2367 | GCUCCUUC A CCACCCUA | 3180 | TAGGGTGG GGCTAGCTACAACGA GAAGGAGC | 11929 |
| 2370 | CCUUCACC A CCCUACCG | 3181 | CGGTAGGG GGCTAGCTACAACGA GGTGAAGG | 11930 |
| 2375 | ACCACCCU A CCGGCUCU | 3182 | AGAGCCGG GGCTAGCTACAACGA AGGGTGGT | 11931 |
| 2379 | CCCUACCG G CUCUGUCC | 3183 | GGACAGAG GGCTAGCTACAACGA CGGTAGGG | 11932 |
| 2384 | CCGGCUCU G UCCACUGG | 3184 | CCAGTGGA GGCTAGCTACAACGA AGAGCCGG | 11933 |
| 2388 | CUCUGUCC A CUGGUUUG | 3185 | CAAACCAG GGCTAGCTACAACGA GGACAGAG | 11934 |
| 2392 | GUCCACUG G UUUGAUCC | 3186 | GGATCAAA GGCTAGCTACAACGA CAGTGGAC | 11935 |
| 2397 | CUGGUUUG A UCCAUCUC | 3187 | GAGATGGA GGCTAGCTACAACGA CAAACCAG | 11936 |
| 2401 | UUUGAUCC A UCUCCACC | 3188 | GGTGGAGA GGCTAGCTACAACGA GGATCAAA | 11937 |
| 2407 | CCAUCUCC A CCAGAACA | 3189 | TGTTCTGG GGCTAGCTACAACGA GGAGATGG | 11938 |
| 2413 | CCACCAGA A CAUCGUGG | 3190 | CCACGATG GGCTAGCTACAACGA TCTGGTGG | 11939 |
| 2415 | ACCAGAAC A UCGUGGAC | 3191 | GTCCACGA GGCTAGCTACAACGA GTTCTGGT | 11940 |
| 2418 | AGAACAUC G UGGACGUG | 3192 | CACGTCCA GGCTAGCTACAACGA GATGTTCT | 11941 |
| 2422 | CAUCGUGG A CGUGCAAU | 3193 | ATTGCACG GGCTAGCTACAACGA CCACGATG | 11942 |
| 2424 | UCGUGGAC G UGCAAUAC | 3194 | GTATTGCA GGCTAGCTACAACGA GTCCACGA | 11943 |
| 2426 | GUGGACGU G CAAUACCU | 3195 | AGGTATTG GGCTAGCTACAACGA ACGTCCAC | 11944 |
| 2429 | GACGUGCA A UACCUGUA | 3196 | TACAGGTA GGCTAGCTACAACGA TGCACGTC | 11945 |
| 2431 | CGUGCAAU A CCUGUACG | 3197 | CGTACAGG GGCTAGCTACAACGA ATTGCACG | 11946 |
| 2435 | CAAUACCU G UACGGUGU | 3198 | ACACCGTA GGCTAGCTACAACGA AGGTATTG | 11947 |
| 2437 | AUACCUGU A CGGUGUAG | 3199 | CTACACCG GGCTAGCTACAACGA ACAGGTAT | 11948 |
| 2440 | CCUGUACG G UGUAGGGU | 3200 | ACCCTACA GGCTAGCTACAACGA CGTACAGG | 11949 |
| 2442 | UGUACGGU G UAGGGUCA | 3201 | TGACCCTA GGCTAGCTACAACGA ACCGTACA | 11950 |
| 2447 | GGUGUAGG G UCAGCGGU | 3202 | ACCGCTGA GGCTAGCTACAACGA CCTACACC | 11951 |
| 2451 | UAGGGUCA G CGGUUGUC | 3203 | GACAACCG GGCTAGCTACAACGA TGACCCTA | 11952 |
| 2454 | GGUCAGCG G UUGUCUCC | 3204 | GGAGACAA GGCTAGCTACAACGA CGCTGACC | 11953 |
| 2457 | CAGCGGUU G UCUCCUUC | 3205 | GAAGGAGA GGCTAGCTACAACGA AACCGCTG | 11954 |
| 2466 | UCUCCUUC G CAAUCAAA | 3206 | TTTGATTG GGCTAGCTACAACGA GAAGGAGA | 11955 |
| 2469 | CCUUCGCA A UCAAAUGG | 3207 | CCATTTGA GGCTAGCTACAACGA TGCGAAGG | 11956 |
| 2474 | GCAAUCAA A UGGGAGUA | 3208 | TACTCCCA GGCTAGCTACAACGA TTGATTGC | 11957 |
| 2480 | AAAUGGGA G UAUGUCCU | 3209 | AGGACATA GGCTAGCTACAACGA TCCCATTT | 11958 |
| 2482 | AUGGGAGU A UGUCCUGU | 3210 | ACAGGACA GGCTAGCTACAACGA ACTCCCAT | 11959 |
| 2484 | GGGAGUAU G UCCUGUUG | 3211 | CAACAGGA GGCTAGCTACAACGA ATACTCCC | 11960 |
| 2489 | UAUGUCCU G UUGCUUUU | 3212 | AAAAGCAA GGCTAGCTACAACGA AGGACATA | 11961 |
| 2492 | GUCCUGUU G CUUUUCCU | 3213 | AGGAAAAG GGCTAGCTACAACGA AACAGGAC | 11962 |
| 2508 | UUCUCCUG G CAGACGCG | 3214 | CGCGTCTG GGCTAGCTACAACGA CAGGAGAA | 11963 |
| 2512 | CCUGGCAG A CGCGCGCG | 3215 | CGCGCGCG GGCTAGCTACAACGA CTGCCAGG | 11964 |
| 2514 | UGGCAGAC G CGCGCGUC | 3216 | GACGCGCG GGCTAGCTACAACGA GTCTGCCA | 11965 |
| 2516 | GCAGACGC G CGCGUCUG | 3217 | CAGACGCG GGCTAGCTACAACGA GCGTCTGC | 11966 |
| 2518 | AGACGCGC G CGUCUGUG | 3218 | CACAGACG GGCTAGCTACAACGA GCGCGTCT | 11967 |
| 2520 | ACGCGCGC G UCUGUGCC | 3219 | GGCACAGA GGCTAGCTACAACGA GCGCGCGT | 11968 |
| 2524 | GCGCGUCU G UGCCUGUU | 3220 | AACAGGCA GGCTAGCTACAACGA AGACGCGC | 11969 |
| 2526 | GCGUCUGU G CCUGUUUG | 3221 | CAAACAGG GGCTAGCTACAACGA ACAGACGC | 11970 |
| 2530 | CUGUGCCU G UUUGUGGA | 3222 | TCCACAAA GGCTAGCTACAACGA AGGCACAG | 11971 |
| 2534 | GCCUGUUU G UGGAUGAU | 3223 | ATCATCCA GGCTAGCTACAACGA AAACAGGC | 11972 |
| 2538 | GUUUGUGG A UGAUGCUG | 3224 | CAGCATCA GGCTAGCTACAACGA CCACAAAC | 11973 |
| 2541 | UGUGGAUG A UGCUGUUG | 3225 | CAACAGCA GGCTAGCTACAACGA CATCCACA | 11974 |
| 2543 | UGGAUGAU G CUGUUGGU | 3226 | ACCAACAG GGCTAGCTACAACGA ATCATCCA | 11975 |
| 2546 | AUGAUGCU G UUGGUAGC | 3227 | GCTACCAA GGCTAGCTACAACGA AGCATCAT | 11976 |
| 2550 | UGCUGUUG G UAGCCCAG | 3228 | CTGGGCTA GGCTAGCTACAACGA CAACAGCA | 11977 |
| 2553 | UGUUGGUA G CCCAGGCC | 3229 | GGCCTGGG GGCTAGCTACAACGA TACCAACA | 11978 |
| 2559 | UAGCCCAG G CCGAGGCU | 3230 | ACCCTCGG GGCTAGCTACAACGA CTGGGCTA | 11979 |
| 2565 | AGGCCGAG G CUGCCCUA | 3231 | TAGGGCAG GGCTAGCTACAACGA CTCGGCCT | 11980 |
| 2568 | CCGAGGCU G CCCUAGAG | 3232 | CTCTAGGG GCCTAGCTACAACGA AGCCTCGG | 11981 |
| 2578 | CCUAGAGA A CCUGGUGG | 3233 | CCACCAGG GGCTAGCTACAACGA TCTCTAGG | 11982 |
| 2583 | AGAACCUG G UGGUCCUC | 3234 | GAGGACCA GGCTAGCTACAACGA CAGGTTCT | 11983 |
| 2586 | ACCUGGUG G UCCUCAAU | 3235 | ATTGAGGA GGCTAGCTACAACGA CACCAGGT | 11984 |
| 2593 | GGUCCUCA A UGCAGCAU | 3236 | ATGCTGCA GGCTAGCTACAACGA TGAGGACC | 11985 |
| 2595 | UCCUCAAU G CAGCAUCC | 3237 | GGATGCTG GGCTAGCTACAACGA ATTGAGGA | 11986 |
| 2598 | UCAAUGCA G CAUCCUUG | 3238 | CAAGGATG GGCTAGCTACAACGA TGCATTGA | 11987 |
| 2600 | AAUGCAGC A UCCUUGGC | 3239 | GCCAAGGA GGCTAGCTACAACGA GCTGCATT | 11988 |
| 2607 | CAUCCUUG G CCGGAGUG | 3240 | CACTCCGG GGCTAGCTACAACGA CAAGGATG | 11989 |
| 2613 | UGGCCGGA G UGCAUGGC | 3241 | GCCATGCA GGCTAGCTACAACGA TCCGGCCA | 11990 |
| 2615 | GCCGGAGU G CAUGGCAU | 3242 | ATGCCATG GGCTAGCTACAACGA ACTCCGGC | 11991 |
| 2617 | CGGAGUGC A UGGCAUCC | 3243 | GGATGCCA GGCTAGCTACAACGA GCACTCCG | 11992 |
| 2620 | AGUGCAUG G CAUCCUCU | 3244 | AGAGGATG GGCTAGCTACAACGA CATGCACT | 11993 |
| 2622 | UGCAUGGC A UCCUCUCC | 3245 | GGAGAGGA GGCTAGCTACAACGA GCCATGCA | 11994 |
| 2637 | CCUUCCUC G UGUUCUUC | 3246 | GAAGAACA GGCTAGCTACAACGA GAGGAAGG | 11995 |
| 2639 | UUCCUCGU G UUCUUCUG | 3247 | CAGAAGAA GGCTAGCTACAACGA ACGAGGAA | 11996 |
| 2647 | GUUCUUCU G UGCUGCCU | 3248 | AGGCAGCA GGCTAGCTACAACCA AGAAGAAC | 11997 |
| 2649 | UCUUCUGU G CUGCCUGG | 3249 | CCAGGCAG GGCTAGCTACAACGA ACAGAAGA | 11998 |
| 2652 | UCUGUGCU G CCUGGUAC | 3250 | GTACCAGG GGCTAGCTACAACGA AGCACAGA | 11999 |
| 2657 | GCUGCCUG G UACAUCAA | 3251 | TTGATGTA GGCTAGCTACAACGA CAGGCAGC | 12000 |
| 2659 | UGCCUGGU A CAUCAAAG | 3252 | CTTTGATG GGCTAGCTACAACGA ACCAGGCA | 12001 |
| 2661 | CCUGGUAC A UCAAAGGC | 3253 | GCCTTTGA GGCTAGCTACAACGA GTACCAGG | 12002 |
| 2668 | CAUCAAAG G CAAGCUGG | 3254 | CCAGCTTG GGCTAGCTACAACGA CTTTGATG | 12003 |
| 2672 | AAAGGCAA G CUGGUCCC | 3255 | GGGACCAG GGCTAGCTACAACGA TTGCCTTT | 12004 |
| 2676 | GCAAGCUG G UCCCUGGG | 3256 | CCCAGGGA GGCTAGCTACAACGA CAGCTTGC | 12005 |
| 2685 | UCCCUGGG G CGGCAUAU | 3257 | ATATGCCG GGCTAGCTACAACGA CCCAGGGA | 12006 |
| 2688 | CUGGGGCG G CAUAUGCU | 3258 | AGCATATG GGCTAGCTACAACGA CGCCCCAG | 12007 |
| 2690 | GGGGCGGC A UAUGCUCU | 3259 | AGAGCATA GGCTAGCTACAACGA GCCGCCCC | 12008 |
| 2692 | GGCGGCAU A UGCUCUCU | 3260 | AGAGAGCA GGCTAGCTACAACGA ATGCCGCC | 12009 |
| 2694 | CGGCAUAU G CUCUCUAC | 3261 | GTAGAGAG GGCTAGCTACAACGA ATATGCCG | 12010 |
| 2701 | UGCUCUCU A CGGCGUAU | 3262 | ATACGCCG GGCTAGCTACAACGA AGAGAGCA | 12011 |
| 2704 | UCUCUACG G CGUAUGGC | 3263 | GCCATACG GGCTAGCTACAACGA CGTAGAGA | 12012 |
| 2706 | UCUACGGC G UAUGGCCG | 3264 | CGGCCATA GGCTAGCTACAACGA GCCGTAGA | 12013 |
| 2708 | UACGGCGU A UGGCCGCU | 3265 | AGCGGCCA GGCTAGCTACAACGA ACGCCGTA | 12014 |
| 2711 | GGCGUAUG G CCGCUACU | 3266 | AGTAGCGG GGCTAGCTACAACGA CATACGCC | 12015 |
| 2714 | GUAUGGCC G CUACUCCU | 3267 | AGGAGTAG GGCTAGCTACAACGA GGCCATAC | 12016 |
| 2717 | UGGCCGCU A CUCCUGCU | 3268 | AGCAGGAG GGCTAGCTACAACGA AGCGGCCA | 12017 |
| 2723 | CUACUCCU G CUCCUGCU | 3269 | AGCAGGAG GGCTAGCTACAACGA AGGAGTAG | 12018 |
| 2729 | CUGCUCCU G CUGGCGUU | 3270 | AACGCCAG GGCTAGCTACAACGA AGGAGCAG | 12019 |
| 2733 | UCCUGCUG G CGUUACCA | 3271 | TGGTAACG GGCTAGCTACAACGA CAGCAGGA | 12020 |
| 2735 | CUGCUGGC G UUACCACC | 3272 | GGTGGTAA GGCTAGCTACAACGA GCCAGCAG | 12021 |
| 2738 | CUGGCGUU A CCACCACG | 3273 | CGTGGTGG GGCTAGCTACAACGA AACGCCAG | 12022 |
| 2741 | GCGUUACC A CCACGGGC | 3274 | GCCCGTGG GGCTAGCTACAACGA GGTAACGC | 12023 |
| 2744 | UUACCACC A CGGGCGUA | 3275 | TACGCCCG GGCTAGCTACAACGA GGTGGTAA | 12024 |
| 2748 | CACCACGG G CGUACGCC | 3276 | GGCGTACG GGCTAGCTACAACGA CCGTGGTG | 12025 |
| 2750 | CCACGGGC G UACGCCAU | 3277 | ATGGCGTA GGCTAGCTACAACGA GCCCGTGG | 12026 |
| 2752 | ACGGGCGU A CGCCAUGG | 3278 | CCATGGCG GGCTAGCTACAACGA ACGCCCGT | 12027 |
| 2754 | GGGCGUAC G CCAUGGAC | 3279 | GTCCATGG GGCTAGCTACAACGA GTACGCCC | 12028 |
| 2757 | CGUACGCC A UGGACCGG | 3280 | CCGGTCCA GGCTAGCTACAACGA GGCGTACG | 12029 |
| 2761 | CGCCAUGG A CCGGGAGA | 3281 | TCTCCCGG GGCTAGCTACAACGA CCATGGCG | 12030 |
| 2769 | ACCGGGAG A UGGCCGCA | 3282 | TGCGGCCA GGCTAGCTACAACGA CTCCCGGT | 12031 |
| 2772 | GGGAGAUG G CCGCAUCG | 3283 | CGATGCGG GGCTAGCTACAACGA CATCTCCC | 12032 |
| 2775 | AGAUGGCC G CAUCGUGC | 3284 | GCACGATG GGCTAGCTACAACGA GGCCATCT | 12033 |
| 2777 | AUGGCCGC A UCGUGCGG | 3285 | CCGCACGA GGCTAGCTACAACGA GCGGCCAT | 12034 |
| 2780 | GCCGCAUC G UGCGGAGG | 3286 | CCTCCGCA GGCTAGCTACAACGA GATGCGGC | 12035 |
| 2782 | CGCAUCGU G CGGAGGCG | 3287 | CGCCTCCG GGCTAGCTACAACGA ACGATGCG | 12036 |
| 2788 | GUGCGGAG G CGUGGUUU | 3288 | AAACCACG GGCTAGCTACAACGA CTCCGCAC | 12037 |
| 2790 | GCGGAGGC G UGGUUUUU | 3289 | AAAAACCA GGCTAGCTACAACGA GCCTCCGC | 12038 |
| 2793 | GAGGCGUG G UUUUUGUA | 3290 | TACAAAAA GGCTAGCTACAACGA CACGCCTC | 12039 |
| 2799 | UGGUUUUU G UAGGUCUA | 3291 | TAGACCTA GGCTAGCTACAACGA AAAAACCA | 12040 |
| 2803 | UUUUGUAG G UCUAGCAC | 3292 | GTGCTAGA GGCTAGCTACAACGA CTACAAAA | 12041 |
| 2808 | UAGGUCUA G CACUCUUG | 3293 | CAAGAGTG GGCTAGCTACAACGA TAGACCTA | 12042 |
| 2810 | GGUCUAGC A CUCUUGAC | 3294 | GTCAAGAG GGCTAGCTACAACGA GCTAGACC | 12043 |
| 2817 | CACUCUUG A CCUUGUCA | 3295 | TGACAAGG GGCTAGCTACAACGA CAAGAGTG | 12044 |
| 2822 | UUGACCUU G UCACCAUA | 3296 | TATGGTGA GGCTAGCTACAACGA AAGGTCAA | 12045 |
| 2825 | ACCUUGUC A CCAUACUA | 3297 | TAGTATGG GGCTAGCTACAACGA GACAAGGT | 12046 |
| 2828 | UUGUCACC A UACUACAA | 3298 | TTGTAGTA GGCTAGCTACAACGA GGTGACAA | 12047 |
| 2830 | GUCACCAU A CUACAAAG | 3299 | CTTTGTAG GGCTAGCTACAACGA ATGGTGAC | 12048 |
| 2833 | ACCAUACU A CAAAGUGU | 3300 | ACACTTTG GGCTAGCTACAACGA AGTATGGT | 12049 |
| 2838 | ACUACAAA G UGUUCCUC | 3301 | GAGGAACA GGCTAGCTACAACGA TTTGTAGT | 12050 |
| 2840 | UACAAAGU G UUCCUCGC | 3302 | GCGAGGAA GGCTAGCTACAACGA ACTTTGTA | 12051 |
| 2847 | UGUUCCUC G CUAGGCUC | 3303 | GAGCCTAG GGCTAGCTACAACGA GAGGAACA | 12052 |
| 2852 | CUCGCUAG G CUCAUAUG | 3304 | CATATGAG GGCTAGCTACAACGA CTAGCGAG | 12053 |
| 2856 | CUAGGCUC A UAUGGUGG | 3305 | CCACCATA GGCTAGCTACAACGA GAGCCTAG | 12054 |
| 2858 | AGGCUCAU A UGGUGGUU | 3306 | AACCACCA GGCTAGCTACAACGA ATGAGCCT | 12055 |
| 2861 | CUCAUAUG G UGGUUGCA | 3307 | TGCAACCA GGCTAGCTACAACGA CATATGAG | 12056 |
| 2864 | AUAUGGUG G UUGCAAUA | 3308 | TATTGCAA GGCTAGCTACAACGA CACCATAT | 12057 |
| 2867 | UGGUGGUU G CAAUACCU | 3309 | AGGTATTG GGCTAGCTACAACGA AACCACCA | 12058 |
| 2870 | UGGUUGCA A UACCUUAU | 3310 | ATAAGGTA GGCTAGCTACAACGA TGCAACCA | 12059 |
| 2872 | GUUGCAAU A CCUUAUCA | 3311 | TGATAAGG GGCTAGCTACAACGA ATTGCAAC | 12060 |
| 2877 | AAUACCUU A UCACCAGA | 3312 | TCTGGTGA GGCTAGCTACAACGA AAGGTATT | 12061 |
| 2880 | ACCUUAUC A CCAGAGCC | 3313 | GGCTCTGG GGCTAGCTACAACGA GATAAGGT | 12062 |
| 2886 | UCACCAGA G CCGAGGCG | 3314 | CGCCTCGG GGCTAGCTACAACGA TCTGGTGA | 12063 |
| 2892 | GAGCCGAG G CGCAGUUG | 3315 | CAACTGCG GGCTAGCTACAACGA CTCGGCTC | 12064 |
| 2894 | GCCGAGGC G CAGUUGCA | 3316 | TGCAACTG GGCTAGCTACAACGA GCCTCGGC | 12065 |
| 2897 | GAGGCGCA G UUGCAAGU | 3317 | ACTTGCAA GGCTAGCTACAACGA TGCGCCTC | 12066 |
| 2900 | GCGCAGUU G CAAGUGUG | 3318 | CACACTTG GGCTAGCTACAACGA AACTGCGC | 12067 |
| 2904 | AGUUGCAA G UGUGGAUC | 3319 | GATCCACA GGCTAGCTACAACGA TTGCAACT | 12068 |
| 2906 | UUGCAAGU G UGGAUCCC | 3320 | GGGATCCA GGCTAGCTACAACGA ACTTGCAA | 12069 |
| 2910 | AAGUGUGG A UCCCCCCC | 3321 | GGGGGGGA GGCTAGCTACAACGA CCACACTT | 12070 |
| 2923 | CCCCCUCA A CGUUCGGG | 3322 | CCCGAACG GGCTAGCTACAACGA TGAGGGGG | 12071 |
| 2925 | CCCUCAAC G UUCGGGGG | 3323 | CCCCCGAA GGCTAGCTACAACGA GTTGAGGG | 12072 |
| 2936 | CGGGGGGG G CGCGGUGC | 3324 | GCACCGCG GGCTAGCTACAACGA CCCCCCCG | 12073 |
| 2938 | GGGGGGGC G CGGUGCCA | 3325 | TGGCACCG GGCTAGCTACAACGA GCCCCCCC | 12074 |
| 2941 | GGGGCGCG G UGCCAUCA | 3326 | TGATGGCA GGCTAGCTACAACGA CGCGCCCC | 12075 |
| 2943 | GGCGCGGU G CCAUCAUU | 3327 | AATGATGG GGCTAGCTACAACGA ACCGCGCC | 12076 |
| 2946 | GCGGUGCC A UCAUUCUC | 3328 | GAGAATGA GGCTAGCTACAACGA GGCACCGC | 12077 |
| 2949 | GUGCCAUC A UUCUCCUC | 3329 | GAGCAGAA GGCTAGCTACAACGA GATGGCAC | 12078 |
| 2958 | UUCUCCUC A CGUGUGUG | 3330 | CACACACG GGCTAGCTACAACGA GAGGAGAA | 12079 |
| 2960 | CUCCUCAC G UGUGUGGU | 3331 | ACCACACA GGCTAGCTACAACGA GTGAGGAG | 12080 |
| 2962 | CCUCACGU G UGUGGUCC | 3332 | GGACCACA GGCTAGCTACAACGA ACGTGAGG | 12081 |
| 2964 | UCACGUGU G UGGUCCAC | 3333 | GTGGACCA GGCTAGCTACAACGA ACACGTGA | 12082 |
| 2967 | CGUGUGUG G UCCACCCA | 3334 | TGGGTGGA GGCTAGCTACAACGA CACACACG | 12083 |
| 2971 | UGUGGUCC A CCCAGAGC | 3335 | GCTCTGGG GGCTAGCTACAACGA GGACCACA | 12084 |
| 2978 | CACCCAGA G CUAAUCUU | 3336 | AAGATTAG GGCTAGCTACAACGA TCTGGGTG | 12085 |
| 2982 | CAGAGCUA A UCUUUGAC | 3337 | GTCAAAGA GGCTAGCTACAACGA TAGCTCTG | 12086 |
| 2989 | AAUCUUUG A CAUCACCA | 3338 | TGGTGATG GGCTAGCTACAACGA CAAAGATT | 12087 |
| 2991 | UCUUUGAC A UCACCAAA | 3339 | TTTGGTGA GGCTAGCTACAACGA GTCAAAGA | 12088 |
| 2994 | UUGACAUC A CCAAAAUU | 3340 | AATTTTGG GGCTAGCTACAACGA GATGTCAA | 12089 |
| 3000 | UCACCAAA A UUAUGCUC | 3341 | GAGCATAA GGCTAGCTACAACGA TTTGGTGA | 12090 |
| 3003 | CCAAAAUU A UGCUCGCC | 3342 | GGCGAGCA GGCTAGCTACAACGA AATTTTGG | 12091 |
| 3005 | AAAAUUAU G CUCGCCAU | 3343 | ATGGCGAG GGCTAGCTACAACGA ATAATTTT | 12092 |
| 3009 | UUAUGCUC G CCAUACUC | 3344 | GAGTATGG GGCTAGCTACAACGA GAGCATAA | 12093 |
| 3012 | UGCUCGCC A UACUCGGC | 3345 | GCCGAGTA GGCTAGCTACAACGA GGCGAGCA | 12094 |
| 3014 | CUCGCCAU A CUCGGCCC | 3346 | GGGCCGAG GGCTAGCTACAACGA ATGGCGAG | 12095 |
| 3019 | CAUACUCG G CCCGCUCA | 3347 | TGAGCGGG GGCTAGCTACAACGA CGAGTATG | 12096 |
| 3023 | CUCGGCCC G CUCAUGGU | 3348 | ACCATGAG GGCTAGCTACAACGA GGGCCGAG | 12097 |
| 3027 | GCCCGCUC A UGGUGCUC | 3349 | GAGCACCA GGCTAGCTACAACGA GAGCGGGC | 12098 |
| 3030 | CGCUCAUG G UGCUCCAG | 3350 | CTGGAGCA GGCTAGCTACAACGA CATGAGCG | 12099 |
| 3032 | CUCAUGGU G CUCCAGGC | 3351 | GCCTGGAG GGCTAGCTACAACGA ACCATGAG | 12100 |
| 3039 | UGCUCCAG G CUGGUAUA | 3352 | TATACCAG GGCTAGCTACAACGA CTGGAGCA | 12101 |
| 3043 | CCAGGCUG G UAUAGCAA | 3353 | TTGCTATA GGCTAGCTACAACGA CAGCCTGG | 12102 |
| 3045 | AGGCUGGU A UAGCAAAA | 3354 | TTTTGCTA GGCTAGCTACAACGA ACCAGCCT | 12103 |
| 3048 | CUGGUAUA G CAAAAGUG | 3355 | CACTTTTG GGCTAGCTACAACGA TATACCAG | 12104 |
| 3054 | UAGCAAAA G UGCCGGAC | 3356 | GTCCGGCA GGCTAGCTACAACGA TTTTGCTA | 12105 |
| 3056 | GCAAAAGU G CCGGACUU | 3357 | AAGTCCGG GGCTAGCTACAACGA ACTTTTGC | 12106 |
| 3061 | AGUGCCGG A CUUUGUGC | 3358 | GCACAAAG GGCTAGCTACAACGA CCGGCACT | 12107 |
| 3066 | CGGACUUU G UGCGGGCU | 3359 | AGCCCGCA GGCTAGCTACAACGA AAAGTCCG | 12108 |
| 3068 | GACUUUGU G CGGGCUCA | 3360 | TGAGCCCG GGCTAGCTACAACGA ACAAAGTC | 12109 |
| 3072 | UUGUGCGG G CUCAAGGG | 3361 | CCCTTGAG GGCTAGCTACAACGA CCGCACAA | 12110 |
| 3081 | CUCAAGGG G UCAUCCGU | 3362 | ACGGATGA GGCTAGCTACAACGA CCCTTGAG | 12111 |
| 3084 | AAGGGGUC A UCCGUGAA | 3363 | TTCACGGA GGCTAGCTACAACGA GACCCCTT | 12112 |
| 3088 | GGUCAUCC G UGAAUGCA | 3364 | TGCATTCA GGCTAGCTACAACGA GGATGACC | 12113 |
| 3092 | AUCCGUGA A UGCAUUUU | 3365 | AAAATGCA GGCTAGCTACAACGA TCACGGAT | 12114 |
| 3094 | CCGUGAAU G CAUUUUGG | 3366 | CCAAAATG GGCTAGCTACAACGA ATTCACGG | 12115 |
| 3096 | GUGAAUGC A UUUUGGUG | 3367 | CACCAAAA GGCTAGCTACAACGA GCATTCAC | 12116 |
| 3102 | GCAUUUUG G UGCGGAAA | 3368 | TTTCCGCA GGCTAGCTACAACGA CAAAATGC | 12117 |
| 3104 | AUUUUGGU G CGGAAAGU | 3369 | ACTTTCCG GGCTAGCTACAACGA ACCAAAAT | 12118 |
| 3111 | UGCGGAAA G UCGGUGGG | 3370 | CCCACCGA GGCTAGCTACAACGA TTTCCGCA | 12119 |
| 3115 | GAAAGUCG G UGGGGGGC | 3371 | GCCCCCCA GGCTAGCTACAACGA CGACTTTC | 12120 |
| 3122 | GGUGGGGG G CAAUAUGU | 3372 | ACATATTG GGCTAGCTACAACGA CCCCCACC | 12121 |
| 3125 | GGGGGGCA A UAUGUCCA | 3373 | TGGACATA GGCTAGCTACAACGA TGCCCCCC | 12122 |
| 3127 | GGGGCAAU A UGUCCAAA | 3374 | TTTGGACA GGCTAGCTACAACGA ATTGCCCC | 12123 |
| 3129 | GGCAAUAU G UCCAAAUG | 3375 | CATTTGGA GGCTAGCTACAACGA ATATTGCC | 12124 |
| 3135 | AUGUCCAA A UGGCCUUC | 3376 | GAAGGCCA GGCTAGCTACAACGA TTGGACAT | 12125 |
| 3138 | UCCAAAUG G CCUUCAUG | 3377 | CATGAAGG GGCTAGCTACAACGA CATTTGGA | 12126 |
| 3144 | UGGCCUUC A UGAAGUUG | 3378 | CAACTTCA GGCTAGCTACAACGA GAAGGCCA | 12127 |
| 3149 | UUCAUGAA G UUGGCCCA | 3379 | TCGGCCAA GGCTAGCTACAACGA TTCATGAA | 12128 |
| 3153 | UGAAGUUG G CCGAAUUG | 3380 | CAATTCGG GGCTAGCTACAACGA CAACTTCA | 12129 |
| 3158 | UUGGCCGA A UUGAAAGG | 3381 | CCTTTCAA GGCTAGCTACAACGA TCGGCCAA | 12130 |
| 3166 | AUUGAAAG G UACGUCCG | 3382 | CGGACGTA GGCTAGCTACAACGA CTTTCAAT | 12131 |
| 3168 | UGAAAGGU A CGUCCGUC | 3383 | GACGGACG GGCTAGCTACAACGA ACCTTTCA | 12132 |
| 3170 | AAAGGUAC G UCCGUCUA | 3384 | TAGACGGA GGCTAGCTACAACGA GTACCTTT | 12133 |
| 3174 | GUACGUCC G UCUAUGAC | 3385 | GTCATAGA GGCTAGCTACAACGA GGACGTAC | 12134 |
| 3178 | GUCCGUCU A UGACCACC | 3386 | GGTGGTCA GGCTAGCTACAACGA AGACGGAC | 12135 |
| 3181 | CGUCUAUG A CCACCUCA | 3387 | TGAGGTGG GGCTAGCTACAACGA CATAGACG | 12136 |
| 3184 | CUAUGACC A CCUCACUC | 3388 | GAGTGAGG GGCTAGCTACAACGA GGTCATAG | 12137 |
| 3189 | ACCACCUC A CUCCACUG | 3389 | CAGTGGAG GGCTAGCTACAACGA GAGGTGGT | 12138 |
| 3194 | CUCACUCC A CUGCAGGA | 3390 | TCCTGCAG GGCTAGCTACAACGA GGAGTGAG | 12139 |
| 3197 | ACUCCACU G CAGGACUG | 3391 | CAGTCCTG GGCTAGCTACAACGA AGTGGAGT | 12140 |
| 3202 | ACUGCAGG A CUGGGCCC | 3392 | GGGCCCAG GGCTAGCTACAACGA CCTGCAGT | 12141 |
| 3207 | AGGACUGG G CCCACACA | 3393 | TGTGTGGG GGCTAGCTACAACGA CCAGTCCT | 12142 |
| 3211 | CUGGGCCC A CACAGGUC | 3394 | GACCTGTG GGCTAGCTACAACGA GGGCCCAG | 12143 |
| 3213 | GGGCCCAC A CAGGUCUA | 3395 | TAGACCTG GGCTAGCTACAACGA GTGGGCCC | 12144 |
| 3217 | CCACACAG G UCUACGAG | 3396 | CTCGTAGA GGCTAGCTACAACGA CTGTGTGG | 12145 |
| 3221 | ACAGGUCU A CGAGACCU | 3397 | AGGTCTCG GGCTAGCTACAACGA AGACCTGT | 12146 |
| 3226 | UCUACGAG A CCUGGCGG | 3398 | CCGCCAGG GGCTAGCTACAACGA CTCGTAGA | 12147 |
| 3231 | GAGACCUG G CGGUAGCG | 3399 | CGCTACCG GGCTAGCTACAACGA CAGGTCTC | 12148 |
| 3234 | ACCUGGCG G UAGCGGUC | 3400 | GACCGCTA GGCTAGCTACAACGA CGCCAGGT | 12149 |
| 3237 | UGGCGGUA G CGGUCGAG | 3401 | CTCGACCG GGCTAGCTACAACGA TACCGCCA | 12150 |
| 3240 | CGGUAGCG G UCGAGCCC | 3402 | GGGCTCGA GGCTAGCTACAACGA CGCTACCG | 12151 |
| 3245 | GCGGUCGA G CCCGUCGU | 3403 | ACGACGGG GGCTAGCTACAACGA TCGACCGC | 12152 |
| 3249 | UCGAGCCC G UCGUCUUC | 3404 | GAAGACGA GGCTAGCTACAACGA GGGCTCGA | 12153 |
| 3252 | AGCCCGUC G UCUUCUCC | 3405 | GGAGAAGA GGCTAGCTACAACGA GACGGGCT | 12154 |
| 3262 | CUUCUCCG A CAUGGAAA | 3406 | TTTCCATG GGCTAGCTACAACGA CGGAGAAG | 12155 |
| 3264 | UCUCCGAC A UGGAAAUC | 3407 | GATTTCCA GGCTAGCTACAACGA GTCGGAGA | 12156 |
| 3270 | ACAUGGAA A UCAAGAUC | 3408 | GATCTTGA GGCTAGCTACAACGA TTCCATGT | 12157 |
| 3276 | AAAUCAAG A UCAUCACC | 3409 | GGTGATGA GGCTAGCTACAACGA CTTGATTT | 12158 |
| 3279 | UCAAGAUC A UCACCUGG | 3410 | CCAGGTGA GGCTAGCTACAACGA GATCTTGA | 12159 |
| 3282 | AGAUCAUC A CCUGGGGG | 3411 | CCCCCAGG GGCTAGCTACAACGA GATGATCT | 12160 |
| 3295 | GGGGGGAG A CACCGCGG | 3412 | CCGCGGTG GGCTAGCTACAACGA CTCCCCCC | 12161 |
| 3297 | GGGGAGAC A CCGCGGCG | 3413 | CGCCGCGG GGCTAGCTACAACGA GTCTCCCC | 12162 |
| 3300 | GAGACACC G CGGCGUGU | 3414 | ACACGCCG GGCTAGCTACAACGA GGTGTCTC | 12163 |
| 3303 | ACACCGCG G CGUGUGGG | 3415 | CCCACACG GGCTAGCTACAACGA CGCGGTGT | 12164 |
| 3305 | ACCGCGGC G UGUGGGGA | 3416 | TCCCCACA GGCTAGCTACAACGA GCCGCGGT | 12165 |
| 3307 | CGCGGCGU G UGGGGACA | 3417 | TGTCCCCA GGCTAGCTACAACGA ACGCCGCG | 12166 |
| 3313 | GUGUGGGG A CAUCAUUA | 3418 | TAATGATG GGCTAGCTACAACGA CCCCACAC | 12167 |
| 3315 | GUGGGGAC A UCAUUAUG | 3419 | CATAATGA GGCTAGCTACAACGA GTCCCCAC | 12168 |
| 3318 | GGGACAUC A UUAUGGGU | 3420 | ACCCATAA GGCTAGCTACAACGA GATGTCCC | 12169 |
| 3321 | ACAUCAUU A UGGGUCUA | 3421 | TAGACCCA GGCTAGCTACAACGA AATGATGT | 12170 |
| 3325 | CAUUAUGG G UCUACCUG | 3422 | CAGGTAGA GGCTAGCTACAACGA CCATAATG | 12171 |
| 3329 | AUGGGUCU A CCUGUCUC | 3423 | GAGACAGG GGCTAGCTACAACGA AGACCCAT | 12172 |
| 3333 | GUCUACCU G UCUCCGCC | 3424 | GGCGGAGA GGCTAGCTACAACGA AGGTAGAC | 12173 |
| 3339 | CUGUCUCC G CCCGAAGG | 3425 | CCTTCGGG GGCTAGCTACAACGA GGAGACAG | 12174 |
| 3357 | GGAGGGAG A UACUCCUA | 3426 | TAGGAGTA GGCTAGCTACAACGA CTCCCTCC | 12175 |
| 3359 | AGGGAGAU A CUCCUAGG | 3427 | CCTAGGAG GGCTAGCTACAACGA ATCTCCCT | 12176 |
| 3368 | CUCCUAGG A CCAGCCGA | 3428 | TCGGCTGG GGCTAGCTACAACGA CCTAGGAG | 12177 |
| 3372 | UAGGACCA G CCGACAGU | 3429 | ACTGTCGG GGCTAGCTACAACGA TGGTCCTA | 12178 |
| 3376 | ACCAGCCG A CAGUCUUG | 3430 | CAAGACTG GGCTAGCTACAACGA CGGCTGGT | 12179 |
| 3379 | AGCCGACA G UCUUGAGG | 3431 | CCTCAAGA GGCTAGCTACAACGA TGTCGGCT | 12180 |
| 3389 | CUUGAGGG G CAGGGGUG | 3432 | CACCCCTG GGCTAGCTACAACGA CCCTCAAG | 12181 |
| 3395 | GGGCAGGG G UGGCGACU | 3433 | AGTCGCCA GGCTAGCTACAACGA CCCTGCCC | 12182 |
| 3398 | CAGGGGUG G CGACUCCU | 3434 | AGGAGTCG GGCTAGCTACAACGA CACCCCTG | 12183 |
| 3401 | GGGUGGCG A CUCCUCGC | 3435 | GCGAGGAG GGCTAGCTACAACGA CGCCACCC | 12184 |
| 3408 | GACUCCUC G CGCCCAUU | 3436 | AATGGGCG GGCTAGCTACAACGA GAGGAGTC | 12185 |
| 3410 | CUCCUCGC G CCCAUUAC | 3437 | GTAATGGG GGCTAGCTACAACGA GCGAGGAG | 12186 |
| 3414 | UCGCGCCC A UUACGGCC | 3438 | GGCCGTAA GGCTAGCTACAACGA GGGCGCGA | 12187 |
| 3417 | CGCCCAUU A CGGCCUAC | 3439 | GTAGGCCG GGCTAGCTACAACGA AATGGGCG | 12188 |
| 3420 | CCAUUACG G CCUACUCC | 3440 | GGAGTAGG GGCTAGCTACAACGA CGTAATGG | 12189 |
| 3424 | UACGGCCU A CUCCCAAC | 3441 | GTTGGGAG GGCTAGCTACAACGA AGGCCGTA | 12190 |
| 3431 | UACUCCCA A CAGACGCG | 3442 | CGCGTCTG GGCTAGCTACAACGA TGGGAGTA | 12191 |
| 3435 | CCCAACAG A CGCGGGGC | 3443 | GCCCCGCG GGCTAGCTACAACGA CTGTTGGG | 12192 |
| 3437 | CAACAGAC G CGGGGCCU | 3444 | AGGCCCCG GGCTAGCTACAACGA GTCTGTTG | 12193 |
| 3442 | GACGCGGG G CCUGUUUG | 3445 | CAAACAGG GGCTAGCTACAACGA CCCGCGTC | 12194 |
| 3446 | CGGGGCCU G UUUGGCUG | 3446 | CAGCCAAA GGCTAGCTACAACGA AGGCCCCG | 12195 |
| 3451 | CCUGUUUG G CUGCAUUA | 3447 | TAATGCAG GGCTAGCTACAACGA CAAACAGG | 12196 |
| 3454 | GUUUGGCU G CAUUAUCA | 3448 | TGATAATG GGCTAGCTACAACGA AGCCAAAC | 12197 |
| 3456 | UUGGCUGC A UUAUCACC | 3449 | GGTGATAA GGCTAGCTACAACGA GCAGCCAA | 12198 |
| 3459 | GCUGCAUU A UCACCAGC | 3450 | GCTGGTGA GGCTAGCTACAACGA AATGCAGC | 12199 |
| 3462 | GCAUUAUC A CCAGCCUC | 3451 | GAGGCTGG GGCTAGCTACAACGA GATAATGC | 12200 |
| 3466 | UAUCACCA G CCUCACGG | 3452 | CCGTGAGG GGCTAGCTACAACGA TGGTGATA | 12201 |
| 3471 | CCAGCCUC A CGGGCCGG | 3453 | CCGGCCCG GGCTAGCTACAACGA GAGGCTGG | 12202 |
| 3475 | CCUCACGG G CCGGGACA | 3454 | TGTCCCGG GGCTAGCTACAACGA CCGTGAGG | 12203 |
| 3481 | GGGCCGGG A CAAGAACC | 3455 | GGTTCTTG GGCTAGCTACAACGA CCCGGCCC | 12204 |
| 3487 | GGACAAGA A CCAAGUCG | 3456 | CGACTTGG GGCTAGCTACAACGA TCTTGTCC | 12205 |
| 3492 | AGAACCAA G UCGAGGGG | 3457 | CCCCTCGA GGCTAGCTACAACGA TTGGTTCT | 12206 |
| 3504 | AGGGGGAA G UUCAAGUG | 3458 | CACTTGAA GGCTAGCTACAACGA TTCCCCCT | 12207 |
| 3510 | AAGUUCAA G UGGUUUCC | 3459 | GGAAACCA GGCTAGCTACAACGA TTGAACTT | 12208 |
| 3513 | UUCAAGUG G UUUCCACC | 3460 | GGTGGAAA GGCTAGCTACAACGA CACTTGAA | 12209 |
| 3519 | UGGUUUCC A CCGCGACG | 3461 | CGTCGCGG GGCTAGCTACAACGA GGAAACCA | 12210 |
| 3522 | UUUCCACC G CGACGCAG | 3462 | CTGCGTCG GGCTAGCTACAACGA GGTGGAAA | 12211 |
| 3525 | CCACCGCG A CGCAGUCU | 3463 | AGACTGCG GGCTAGCTACAACGA CGCGGTGG | 12212 |
| 3527 | ACCGCGAC G CAGUCUUU | 3464 | AAAGACTG GGCTAGCTACAACGA GTCGCGGT | 12213 |
| 3530 | GCGACGCA G UCUUUCCU | 3465 | AGGAAAGA GGCTAGCTACAACGA TGCGTCGC | 12214 |
| 3540 | CUUUCCUA G CGACCUGC | 3466 | GCAGGTCC GGCTAGCTACAACGA TAGGAAAG | 12215 |
| 3543 | UCCUAGCG A CCUGCGUC | 3467 | GACGCAGG GGCTAGCTACAACGA CGCTAGGA | 12216 |
| 3547 | AGCGACCU G CGUCAACG | 3468 | CGTTGACG GGCTAGCTACAACGA AGGTCGCT | 12217 |
| 3549 | CGACCUGC G UCAACGGC | 3469 | GCCGTTGA GGCTAGCTACAACGA GCAGGTCG | 12218 |
| 3553 | CUGCGUCA A CGGCGUGU | 3470 | ACACGCCG GGCTAGCTACAACGA TGACGCAG | 12219 |
| 3556 | CGUCAACG G CGUGUGCU | 3471 | AGCACACG GGCTAGCTACAACGA CGTTGACG | 12220 |
| 3558 | UCAACGGC G UGUGCUGG | 3472 | CCAGCACA GGCTAGCTACAACGA GCCGTTGA | 12221 |
| 3560 | AACGGCGU G UGCUGGAC | 3473 | GTCCAGCA GGCTAGCTACAACGA ACGCCGTT | 12222 |
| 3562 | CGGCGUGU G CUGGACUG | 3474 | CAGTCCAG GGCTAGCTACAACGA ACACGCCG | 12223 |
| 3567 | UGUGCUGG A CUGUCUAC | 3475 | GTAGACAG GGCTAGCTACAACGA CCAGCACA | 12224 |
| 3570 | GCUGGACU G UCUACCAC | 3476 | GTGGTAGA GGCTAGCTACAACGA AGTCCAGC | 12225 |
| 3574 | GACUGUCU A CCACGGCG | 3477 | CGCCGTGG GGCTAGCTACAACGA AGACAGTC | 12226 |
| 3577 | UGUCUACC A CGGCGCCG | 3478 | CGGCGCCG GGCTAGCTACAACGA GGTAGACA | 12227 |
| 3580 | CUACCACG G CGCCGGCU | 3479 | AGCCGGCG GGCTAGCTACAACGA CGTGGTAG | 12228 |
| 3582 | ACCACGGC G CCGGCUCA | 3480 | TGAGCCGG GGCTAGCTACAACGA GCCGTGGT | 12229 |
| 3586 | CGGCGCCG G CUCAAAGA | 3481 | TCTTTGAG GGCTAGCTACAACGA CGGCGCCG | 12230 |
| 3594 | GCUCAAAG A CCCUAGCC | 3482 | GGCTAGGG GGCTAGCTACAACGA CTTTGAGC | 12231 |
| 3600 | AGACCCUA G CCGGCCCA | 3483 | TGGGCCGG GGCTAGCTACAACGA TAGGGTCT | 12232 |
| 3604 | CCUAGCCG G CCCAAAGG | 3484 | CCTTTGGG GGCTAGCTACAACGA CGGCTAGG | 12233 |
| 3613 | CCCAAAGG G UCCAAUCA | 3485 | TGATTGGA GGCTAGCTACAACGA CCTTTGGG | 12234 |
| 3618 | AGGGUCCA A UCACCCAA | 3486 | TTGGGTGA GGCTAGCTACAACGA TGGACCCT | 12235 |
| 3621 | GUCCAAUC A CCCAAAUG | 3487 | CATTTGGG GGCTAGCTACAACGA GATTGGAC | 12236 |
| 3627 | UCACCCAA A UGUACACC | 3488 | GGTGTACA GGCTAGCTACAACGA TTGGGTGA | 12237 |
| 3629 | ACCCAAAU G UACACCAA | 3489 | TTGGTGTA GGCTAGCTACAACGA ATTTGGGT | 12238 |
| 3631 | CCAAAUGU A CACCAAUG | 3490 | CATTGGTG GGCTAGCTACAACGA ACATTTGG | 12239 |
| 3633 | AAAUGUAC A CCAAUGUA | 3491 | TACATTGG GGCTAGCTACAACGA GTACATTT | 12240 |
| 3637 | GUACACCA A UGUAGACC | 3492 | GGTCTACA GGCTAGCTACAACGA TGGTGTAC | 12241 |
| 3639 | ACACCAAU G UAGACCAG | 3493 | CTGGTCTA GGCTAGCTACAACGA ATTGGTGT | 12242 |
| 3643 | CAAUGUAG A CCAGGACC | 3494 | GGTCCTGG GGCTAGCTACAACGA CTACATTG | 12243 |
| 3649 | AGACCAGG A CCUCGUCG | 3495 | CGACGAGG GGCTAGCTACAACGA CCTGGTCT | 12244 |
| 3654 | AGGACCUC G UCGGAUGG | 3496 | CCATCCGA GGCTAGCTACAACGA GAGGTCCT | 12245 |
| 3659 | CUCGUCGG A UGGCCGGC | 3497 | GCCGGCCA GGCTAGCTACAACGA CCGACGAG | 12246 |
| 3662 | GUCGGAUG G CCGGCGCC | 3498 | GGCGCCGG GGCTAGCTACAACGA CATCCGAC | 12247 |
| 3666 | GAUGGCCG G CGCCCCCC | 3499 | GGGGGGCG GGCTAGCTACAACGA CGGCCATC | 12248 |
| 3668 | UGGCCGGC G CCCCCCGG | 3500 | CCGGGGGG GGCTAGCTACAACGA GCCGGCCA | 12249 |
| 3678 | CCCCCGGA G CGCGGUCC | 3501 | GGACCGCG GGCTAGCTACAACGA TCCGGGGG | 12250 |
| 3680 | CCCGGAGC G CGGUCCUU | 3502 | AAGGACCG GGCTAGCTACAACGA GCTCCGGG | 12251 |
| 3683 | GGAGCGCG G UCCUUGAC | 3503 | GTCAAGGA GGCTAGCTACAACGA CGCGCTCC | 12252 |
| 3690 | GGUCCUUG A CACCAUGC | 3504 | GCATGGTG GGCTAGCTACAACGA CAAGGACC | 12253 |
| 3692 | UCCUUGAC A CCAUGCAC | 3505 | GTGCATGG GGCTAGCTACAACGA GTCAAGGA | 12254 |
| 3695 | UUGACACC A UGCACCUG | 3506 | CAGGTGCA GGCTAGCTACAACGA GGTGTCAA | 12255 |
| 3697 | GACACCAU G CACCUGCG | 3507 | CGCAGGTG GGCTAGCTACAACGA ATGGTGTC | 12256 |
| 3699 | CACCAUGC A CCUGCGGC | 3508 | GCCGCAGG GGCTAGCTACAACGA GCATGGTG | 12257 |
| 3703 | AUGCACCU G CGGCGGCU | 3509 | AGCCGCCG GGCTAGCTACAACGA AGGTGCAT | 12258 |
| 3706 | CACCUGCG G CGGCUCGG | 3510 | CCGAGCCG GGCTAGCTACAACGA CGCAGGTG | 12259 |
| 3709 | CUGCGGCG G CUCGGACC | 3511 | GGTCCGAG GGCTAGCTACAACGA CGCCGCAG | 12260 |
| 3715 | CGGCUCGG A CCUUUACU | 3512 | AGTAAAGG GGCTAGCTACAACGA CCGAGCCG | 12261 |
| 3721 | GGACCUUU A CUUGGUCA | 3513 | TGACCAAG GGCTAGCTACAACGA AAAGGTCC | 12262 |
| 3726 | UUUACUUG G UCACGAGA | 3514 | TCTCGTGA GGCTAGCTACAACGA CAAGTAAA | 12263 |
| 3729 | ACUUGGUC A CGAGACAC | 3515 | GTGTCTCG GGCTAGCTACAACGA GACCAAGT | 12264 |
| 3734 | GUCACGAG A CACGCUGA | 3516 | TCAGCGTG GGCTAGCTACAACGA CTCGTGAC | 12265 |
| 3736 | CACGAGAC A CGCUGAUG | 3517 | CATCAGCG GGCTAGCTACAACGA GTCTCGTG | 12266 |
| 3738 | CGAGACAC G CUGAUGUC | 3518 | GACATCAG GGCTAGCTACAACGA GTGTCTCG | 12267 |
| 3742 | ACACGCUG A UGUCAUUC | 3519 | GAATGACA GGCTAGCTACAACGA CAGCGTGT | 12268 |
| 3744 | ACGCUGAU G UCAUUCCG | 3520 | CGGAATGA GGCTAGCTACAACGA ATCAGCGT | 12269 |
| 3747 | CUGAUGUC A UUCCGGUG | 3521 | CACCGGAA GGCTAGCTACAACGA GACATCAG | 12270 |
| 3753 | UCAUUCCG G UGCGCCGG | 3522 | CCGGCGCA GGCTAGCTACAACGA CGGAATGA | 12271 |
| 3755 | AUUCCGGU G CGCCGGCG | 3523 | CGCCGGCG GGCTAGCTACAACGA ACCGGAAT | 12272 |
| 3757 | UCCGGUGC G CCGGCGGG | 3524 | CCCGCCGG GGCTAGCTACAACGA GCACCGGA | 12273 |
| 3761 | GUGCGCCG G CGGGGUGA | 3525 | TCACCCCG GGCTAGCTACAACGA CGGCGCAC | 12274 |
| 3766 | CCGGCGGG G UGACAGCA | 3526 | TGCTGTCA GGCTAGCTACAACGA CCCGCCGG | 12275 |
| 3769 | GCGGGGUG A CAGCAGGG | 3527 | CCCTGCTG GGCTAGCTACAACGA CACCCCGC | 12276 |
| 3772 | GGGUGACA G CAGGGGGA | 3528 | TCCCCCTG GGCTAGCTACAACGA TGTCACCC | 12277 |
| 3781 | CAGGGGGA G CUUACUAU | 3529 | ATAGTAAG GGCTAGCTACAACGA TCCCCCTG | 12278 |
| 3785 | GGGAGCUU A CUAUCCCC | 3530 | GGGGATAG GGCTAGCTACAACGA AAGCTCCC | 12279 |
| 3788 | AGCUUACU A UCCCCCAG | 3531 | CTGGGGGA GGCTAGCTACAACGA AGTAAGCT | 12280 |
| 3797 | UCCCCCAG G CCCAUCUC | 3532 | GAGATGGG GGCTAGCTACAACGA CTGGGGGA | 12281 |
| 3801 | CCAGGCCC A UCUCCUAC | 3533 | GTAGGAGA GGCTAGCTACAACGA GGGCCTGG | 12282 |
| 3808 | CAUCUCCU A CUUGAAGG | 3534 | CCTTCAAG GGCTAGCTACAACGA AGGAGATG | 12283 |
| 3817 | CUUGAAGG G CUCCUCGG | 3535 | CCGAGGAG GGCTAGCTACAACGA CCTTCAAG | 12284 |
| 3826 | CUCCUCGG G CGGUCCAC | 3536 | GTGGACCG GGCTAGCTACAACGA CCGAGGAG | 12285 |
| 3829 | CUCGGGCG G UCCACUGC | 3537 | GCAGTGGA GGCTAGCTACAACGA CGCCCGAG | 12286 |
| 3833 | GGCGGUCC A CUGCUCUG | 3538 | CAGAGCAG GGCTAGCTACAACGA GGACCGCC | 12287 |
| 3836 | GGUCCACU G CUCUGCCC | 3539 | GGGCAGAG GGCTAGCTACAACGA AGTGGACC | 12288 |
| 3841 | ACUGCUCU G CCCUUCGG | 3540 | CCGAAGGG GGCTAGCTACAACGA AGAGCAGT | 12289 |
| 3851 | CCUUCGGG G CACGUUGU | 3541 | ACAACGTG GGCTAGCTACAACGA CCCGAAGG | 12290 |
| 3853 | UUCGGGGC A CGUUGUGG | 3542 | CCACAACG GGCTAGCTACAACGA GCCCCGAA | 12291 |
| 3855 | CGGGGCAC G UUGUGGGC | 3543 | GCCCACAA GGCTAGCTACAACGA GTGCCCCG | 12292 |
| 3858 | GGCACGUU G UGGGCAUC | 3544 | GATGCCCA GGCTAGCTACAACGA AACGTGCC | 12293 |
| 3862 | CGUUGUGG G CAUCUUCC | 3545 | GGAAGATG GGCTAGCTACAACGA CCACAACG | 12294 |
| 3864 | UUGUGGGC A UCUUCCGG | 3546 | CCGGAAGA GGCTAGCTACAACGA GCCCACAA | 12295 |
| 3873 | UCUUCCGG G CUGCUGUG | 3547 | CACAGCAG GGCTAGCTACAACGA CCGGAAGA | 12296 |
| 3876 | UCCGGGCU G CUGUGUGC | 3548 | GCACACAG GGCTAGCTACAACGA AGCCCGGA | 12297 |
| 3879 | GGGCUGCU G UGUGCACC | 3549 | GGTGCACA GGCTAGCTACAACGA AGCAGCCC | 12298 |
| 3881 | GCUGCUGU G UGCACCCG | 3550 | CGGGTGCA GGCTAGCTACAACGA ACAGCAGC | 12299 |
| 3883 | UGCUGUGU G CACCCGGG | 3551 | CCCGGGTG GGCTAGCTACAACGA ACACAGCA | 12300 |
| 3885 | CUGUGUGC A CCCGGGGG | 3552 | CCCCCGGG GGCTAGCTACAACGA GCACACAG | 12301 |
| 3894 | CCCGGGGG G UUGCGAAG | 3553 | CTTCGCAA GGCTAGCTACAACGA CCCCCGGG | 12302 |
| 3897 | GGGGGGUU G CGAAGGCG | 3554 | CGCCTTCG GGCTAGCTACAACGA AACCCCCC | 12303 |
| 3903 | UUGCGAAG G CGGUGGAC | 3555 | GTCCACCG GGCTAGCTACAACGA CTTCGCAA | 12304 |
| 3906 | CGAAGGCG G UGGACUUU | 3556 | AAAGTCCA GGCTAGCTACAACGA CGCCTTCG | 12305 |
| 3910 | GGCGGUGG A CUUUGUAC | 3557 | GTACAAAG GGCTAGCTACAACGA CCACCGCC | 12306 |
| 3915 | UGGACUUU G UACCCGUU | 3558 | AACGGGTA GGCTAGCTACAACGA AAAGTCCA | 12307 |
| 3917 | GACUUUGU A CCCGUUGA | 3559 | TCAACGGG GGCTAGCTACAACGA ACAAAGTC | 12308 |
| 3921 | UUGUACCC G UUCAGUCU | 3560 | AGACTCAA GGCTAGCTACAACGA GGGTACAA | 12309 |
| 3926 | CCCGUUGA G UCUAUGGA | 3561 | TCCATAGA GGCTAGCTACAACGA TCAACGGG | 12310 |
| 3930 | UUGAGUCU A UGGAAACU | 3562 | AGTTTCCA GGCTAGCTACAACGA AGACTCAA | 12311 |
| 3936 | CUAUGGAA A CUACCAUG | 3563 | CATGGTAG GGCTAGCTACAACGA TTCCATAG | 12312 |
| 3939 | UGGAAACU A CCAUGCGG | 3564 | CCGCATGG GGCTAGCTACAACGA AGTTTCCA | 12313 |
| 3942 | AAACUACC A UGCCGUCC | 3565 | GGACCGCA GGCTAGCTACAACGA GGTAGTTT | 12314 |
| 3944 | ACUACCAU G CGGUCCCC | 3566 | GGGGACCG GGCTAGCTACAACGA ATGGTAGT | 12315 |
| 3947 | ACCAUGCG G UCCCCGGU | 3567 | ACCGGGGA GGCTAGCTACAACGA CGCATGGT | 12316 |
| 3954 | GGUCCCCG G UCUUCACG | 3568 | CGTGAAGA GGCTAGCTACAACGA CGGGGACC | 12317 |
| 3960 | CGGUCUUC A CGGACAAC | 3569 | GTTGTCCG GGCTAGCTACAACGA GAAGACCG | 12318 |
| 3964 | CUUCACGG A CAACUCGU | 3570 | ACGAGTTG GGCTAGCTACAACGA CCGTGAAG | 12319 |
| 3967 | CACGGACA A CUCGUCCC | 3571 | GGGACGAG GGCTAGCTACAACGA TGTCCGTG | 12320 |
| 3971 | GACAACUC G UCCCCCCC | 3572 | GGGGGGGA GGCTAGCTACAACGA GAGTTGTC | 12321 |
| 3981 | CCCCCCCA G CCGUACCG | 3573 | CGGTACGG GGCTAGCTACAACGA TGGGGGGG | 12322 |
| 3984 | CCCCAGCC G UACCGCAG | 3574 | CTGCGGTA GGCTAGCTACAACGA GGCTGGGG | 12323 |
| 3986 | CCAGCCGU A CCGCAGAC | 3575 | GTCTGCGG GGCTAGCTACAACGA ACGGCTGG | 12324 |
| 3989 | GCCGUACC G CAGACAUU | 3576 | AATGTCTG GGCTAGCTACAACGA GGTACGGC | 12325 |
| 3993 | UACCGCAG A CAUUCCAA | 3577 | TTGGAATG GGCTAGCTACAACGA CTGCGGTA | 12326 |
| 3995 | CCGCAGAC A UUCCAAGU | 3578 | ACTTGGAA GGCTAGCTACAACGA GTCTGCGG | 12327 |
| 4002 | CAUUCCAA G UGGCCCAC | 3579 | GTGGGCCA GGCTAGCTACAACGA TTGGAATG | 12328 |
| 4005 | UCCAAGUG G CCCACCUA | 3580 | TAGGTGGG GGCTAGCTACAACGA CACTTGGA | 12329 |
| 4009 | AGUGGCCC A CCUACACG | 3581 | CGTGTAGG GGCTAGCTACAACGA GGGCCACT | 12330 |
| 4013 | GCCCACCU A CACGCUCC | 3582 | GGAGCGTG GGCTAGCTACAACGA AGGTGGGC | 12331 |
| 4015 | CCACCUAC A CGCUCCCA | 3583 | TGGGAGCG GGCTAGCTACAACGA GTAGGTGG | 12332 |
| 4017 | ACCUACAC G CUCCCACU | 3584 | AGTGGGAG GGCTAGCTACAACGA GTGTAGGT | 12333 |
| 4023 | ACGCUCCC A CUGGCAGC | 3585 | GCTGCCAG GGCTAGCTACAACGA GGGAGCGT | 12334 |
| 4027 | UCCCACUG G CAGCGGCA | 3586 | TGCCGCTG GGCTAGCTACAACGA CAGTGGGA | 12335 |
| 4030 | CACUGGCA G CGGCAAGA | 3587 | TCTTGCCG GGCTAGCTACAACGA TGCCAGTG | 12336 |
| 4033 | UGGCAGCG G CAAGAGCA | 3588 | TGCTCTTG GGCTAGCTACAACGA CGCTGCCA | 12337 |
| 4039 | CGGCAAGA G CACUAAGG | 3589 | CCTTAGTG GGCTAGCTACAACGA TCTTGCCG | 12338 |
| 4041 | GCAAGAGC A CUAAGGUA | 3590 | TACCTTAG GGCTAGCTACAACGA GCTCTTGC | 12339 |
| 4047 | GCACUAAG G UACCGGCU | 3591 | AGCCGGTA GGCTAGCTACAACGA CTTAGTGC | 12340 |
| 4049 | ACUAAGGU A CCGGCUGC | 3592 | GCAGCCGG GGCTAGCTACAACGA ACCTTAGT | 12341 |
| 4053 | AGGUACCG G CUGCAUAU | 3593 | ATATGCAG GGCTAGCTACAACGA CGGTACCT | 12342 |
| 4056 | UACCGGCU G CAUAUGCA | 3594 | TGCATATG GGCTAGCTACAACGA AGCCGGTA | 12343 |
| 4058 | CCGGCUGC A UAUGCAGC | 3595 | GCTGCATA GGCTAGCTACAACGA GCAGCCGG | 12344 |
| 4060 | GGCUGCAU A UGCAGCCC | 3596 | GGGCTGCA GGCTAGCTACAACGA ATGCAGCC | 12345 |
| 4062 | CUGCAUAU G CAGCCCAA | 3597 | TTGGGCTG GGCTAGCTACAACGA ATATGCAG | 12346 |
| 4065 | CAUAUGCA G CCCAAGGG | 3598 | CCCTTGGG GGCTAGCTACAACGA TGCATATG | 12347 |
| 4073 | GCCCAAGG G UACAAAGU | 3599 | ACTTTGTA GGCTAGCTACAACGA CCTTGGGC | 12348 |
| 4075 | CCAAGGGU A CAAAGUGC | 3600 | GCACTTTG GGCTAGCTACAACGA ACCCTTGG | 12349 |
| 4080 | GGUACAAA G UGCUCGUC | 3601 | GACGAGCA GGCTAGCTACAACGA TTTGTACC | 12350 |
| 4082 | UACAAAGU G CUCGUCCU | 3602 | AGGACGAG GGCTAGCTACAACGA ACTTTGTA | 12351 |
| 4086 | AAGUGCUC G UCCUAAAU | 3603 | ATTTAGGA GGCTAGCTACAACGA GAGCACTT | 12352 |
| 4093 | CGUCCUAA A UCCGUCCG | 3604 | CGGACGGA GGCTAGCTACAACGA TTAGGACG | 12353 |
| 4097 | CUAAAUCC G UCCGUUAC | 3605 | GTAACGGA GGCTAGCTACAACGA GGATTTAG | 12354 |
| 4101 | AUCCGUCC G UUACCGCC | 3606 | GGCGGTAA GGCTAGCTACAACGA GGACGGAT | 12355 |
| 4104 | CGUCCGUU A CCGCCACC | 3607 | GGTGGCGG GGCTAGCTACAACGA AACGGACG | 12356 |
| 4107 | CCGUUACC G CCACCUUA | 3608 | TAAGGTGG GGCTAGCTACAACGA GGTAACGG | 12357 |
| 4110 | UUACCGCC A CCUUAGGG | 3609 | CCCTAAGG GGCTAGCTACAACGA GGCGGTAA | 12358 |
| 4118 | ACCUUAGG G UUUGGGGC | 3610 | GCCCCAAA GGCTAGCTACAACGA CCTAAGGT | 12359 |
| 4125 | GGUUUGGG G CGUAUAUG | 3611 | CATATACG GGCTAGCTACAACGA CCCAAACC | 12360 |
| 4127 | UUUGGGGC G UAUAUGUC | 3612 | GACATATA GGCTAGCTACAACGA GCCCCAAA | 12361 |
| 4129 | UGGGGCGU A UAUGUCUA | 3613 | TAGACATA GGCTAGCTACAACGA ACGCCCCA | 12362 |
| 4131 | GGGCGUAU A UGUCUAAG | 3614 | CTTAGACA GGCTAGCTACAACGA ATACGCCC | 12363 |
| 4133 | GCGUAUAU G UCUAAGGC | 3615 | GCCTTAGA GGCTAGCTACAACGA ATATACGC | 12364 |
| 4140 | UGUCUAAG G CACACGGU | 3616 | ACCGTGTG GGCTAGCTACAACGA CTTAGACA | 12365 |
| 4142 | UCUAAGGC A CACGGUGU | 3617 | ACACCGTG GGCTAGCTACAACGA GCCTTAGA | 12366 |
| 4144 | UAAGGCAC A CGGUGUCG | 3618 | CGACACCG GGCTAGCTACAACGA GTGCCTTA | 12367 |
| 4147 | GGCACACG G UGUCGAUC | 3619 | GATCGACA GGCTAGCTACAACGA CGTGTGCC | 12368 |
| 4149 | CACACGGU G UCGAUCCU | 3620 | AGGATCGA GGCTAGCTACAACGA ACCGTGTG | 12369 |
| 4153 | CGGUGUCG A UCCUAACA | 3621 | TGTTAGGA GGCTAGCTACAACGA CGACACCG | 12370 |
| 4159 | CGAUCCUA A CAUCAGAA | 3622 | TTCTGATG GGCTAGCTACAACGA TAGGATCG | 12371 |
| 4161 | AUCCUAAC A UCAGAACU | 3623 | AGTTCTGA GGCTAGCTACAACGA GTTAGGAT | 12372 |
| 4167 | ACAUCAGA A CUGGGGUA | 3624 | TACCCCAG GGCTAGCTACAACGA TCTGATGT | 12373 |
| 4173 | GAACUGGG G UAAGGACC | 3625 | GGTCCTTA GGCTAGCTACAACGA CCCAGTTC | 12374 |
| 4179 | GGGUAAGG A CCAUCACC | 3626 | GGTGATGG GGCTAGCTACAACGA CCTTACCC | 12375 |
| 4182 | UAAGGACC A UCACCACG | 3627 | CGTGGTGA GGCTAGCTACAACGA GGTCCTTA | 12376 |
| 4185 | GGACCAUC A CCACGGGC | 3628 | GCCCGTGG GGCTAGCTACAACGA GATGGTCC | 12377 |
| 4188 | CCAUCACC A CGGGCGCC | 3629 | GGCGCCCG GGCTAGCTACAACGA GGTGATGG | 12378 |
| 4192 | CACCACGG G CGCCCCCA | 3630 | TGGGGGCG GGCTAGCTACAACGA CCGTGGTG | 12379 |
| 4194 | CCACGGGC G CCCCCAUC | 3631 | GATGGGGG GGCTAGCTACAACGA GCCCGTGG | 12380 |
| 4200 | GCGCCCCC A UCACGUAC | 3632 | GTACGTGA GGCTAGCTACAACGA GGGGGCGC | 12381 |
| 4203 | CCCCCAUC A CGUACUCC | 3633 | GGAGTACG GGCTAGCTACAACGA GATGGGGG | 12382 |
| 4205 | CCCAUCAC G UACUCCAC | 3634 | GTGGAGTA GGCTAGCTACAACGA GTGATGGG | 12383 |
| 4207 | CAUCACGU A CUCCACCU | 3635 | AGGTGGAG GGCTAGCTACAACGA ACGTGATG | 12384 |
| 4212 | CGUACUCC A CCUAUGGC | 3636 | GCCATAGG GGCTAGCTACAACGA GGAGTACG | 12385 |
| 4216 | CUCCACCU A UGGCAAGU | 3637 | ACTTGCCA GGCTAGCTACAACGA AGGTGGAG | 12386 |
| 4219 | CACCUAUG G CAAGUUCC | 3638 | GGAACTTG GGCTAGCTACAACGA CATAGGTG | 12387 |
| 4223 | UAUGGCAA G UUCCUUGC | 3639 | GCAAGGAA GGCTAGCTACAACGA TTGCCATA | 12388 |
| 4230 | AGUUCCUU G CCGACGGU | 3640 | ACCGTCGG GGCTAGCTACAACGA AAGGAACT | 12389 |
| 4234 | CCUUGCCG A CGGUGGUU | 3641 | AACCACCG GGCTAGCTACAACGA CGGCAAGG | 12390 |
| 4237 | UGCCGACG G UGGUUGCU | 3642 | AGCAACCA GGCTAGCTACAACGA CGTCGGCA | 12391 |
| 4240 | CGACGGUG G UUGCUCUG | 3643 | CAGAGCAA GGCTAGCTACAACGA CACCGTCG | 12392 |
| 4243 | CGGUGGUU G CUCUGGGG | 3644 | CCCCAGAG GGCTAGCTACAACGA AACCACCG | 12393 |
| 4252 | CUCUGGGG G CCCCUAUG | 3645 | CATAGCCG GGCTAGCTACAACGA CCCCAGAG | 12394 |
| 4254 | CUGGGGGC G CCUAUGAC | 3646 | GTCATAGG GGCTAGCTACAACGA GCCCCCAG | 12395 |
| 4258 | GGGCGCCU A UGACAUCA | 3647 | TGATGTCA GGCTAGCTACAACGA AGGCGCCC | 12396 |
| 4261 | CGCCUAUG A CAUCAUAA | 3648 | TTATGATG GGCTAGCTACAACGA CATAGGCG | 12397 |
| 4263 | CCUAUGAC A UCAUAAUG | 3649 | CATTATGA GGCTAGCTACAACGA GTCATAGG | 12398 |
| 4266 | AUGACAUC A UAAUGUGU | 3650 | ACACATTA GGCTAGCTACAACGA GATGTCAT | 12399 |
| 4269 | ACAUCAUA A UGUGUGAU | 3651 | ATCACACA GGCTAGCTACAACGA TATGATGT | 12400 |
| 4271 | AUCAUAAU G UGUGAUGA | 3652 | TCATCACA GGCTAGCTACAACGA ATTATGAT | 12401 |
| 4273 | CAUAAUGU G UGAUGAGU | 3653 | ACTCATCA GGCTAGCTACAACGA ACATTATG | 12402 |
| 4276 | AAUGUGUG A UGAGUGCC | 3654 | GGCACTCA GGCTAGCTACAACGA CACACATT | 12403 |
| 4280 | UGUGAUGA G UGCCACUC | 3655 | GAGTGGCA GGCTAGCTACAACGA TCATCACA | 12404 |
| 4282 | UGAUGAGU G CCACUCAA | 3656 | TTGAGTGG GGCTAGCTACAACGA ACTCATCA | 12405 |
| 4285 | UGAGUGCC A CUCAAUUG | 3657 | CAATTGAG GGCTAGCTACAACGA GGCACTCA | 12406 |
| 4290 | GCCACUCA A UUGACUCG | 3658 | CGAGTCAA GGCTAGCTACAACGA TGAGTGGC | 12407 |
| 4294 | CUCAAUUG A CUCGACUU | 3659 | AAGTCGAG GGCTAGCTACAACGA CAATTGAG | 12408 |
| 4299 | UUGACUCG A CUUCCAUU | 3660 | AATGGAAG GGCTAGCTACAACGA CGAGTCAA | 12409 |
| 4305 | CGACUUCC A UUUUGGGC | 3661 | GCCCAAAA GGCTAGCTACAACGA GGAAGTCG | 12410 |
| 4312 | CAUUUUGG G CAUCGGCA | 3662 | TGCCGATG GGCTAGCTACAACGA CCAAAATG | 12411 |
| 4314 | UUUUGGGC A UCGGCACA | 3663 | TGTGCCGA GGCTAGCTACAACGA GCCCAAAA | 12412 |
| 4318 | GGGCAUCG G CACAGUCC | 3664 | GGACTGTG GGCTAGCTACAACGA CGATGCCC | 12413 |
| 4320 | GCAUCGGC A CAGUCCUG | 3665 | CAGGACTG GGCTAGCTACAACGA GCCGATGC | 12414 |
| 4323 | UCGGCACA G UCCUGGAC | 3666 | GTCCAGGA GGCTAGCTACAACGA TGTGCCGA | 12415 |
| 4330 | AGUCCUGG A CCAAGCGG | 3667 | CCGCTTGG GGCTAGCTACAACGA CCAGGACT | 12416 |
| 4335 | UGGACCAA G CGGAGACG | 3668 | CGTCTCCG GGCTAGCTACAACGA TTGGTCCA | 12417 |
| 4341 | AAGCGGAG A CGGCUGGA | 3669 | TCCAGCCG GGCTAGCTACAACGA CTCCGCTT | 12418 |
| 4344 | CGGAGACG G CUGGAGCG | 3670 | CGCTCCAG GGCTAGCTACAACGA CGTCTCCG | 12419 |
| 4350 | CGGCUGGA G CGCGGCUC | 3671 | GAGCCGCG GGCTAGCTACAACGA TCCAGCCG | 12420 |
| 4352 | GCUGGAGC G CGGCUCGU | 3672 | ACGAGCCG GGCTAGCTACAACGA GCTCCAGC | 12421 |
| 4355 | GGAGCGCG G CUCGUCGU | 3673 | ACGACGAG GGCTAGCTACAACGA CGCGCTCC | 12422 |
| 4359 | CGCGGCUC G UCGUGCUC | 3674 | GAGCACGA GGCTAGCTACAACGA GAGCCGCG | 12423 |
| 4362 | GGCUCGUC G UGCUCGCC | 3675 | GGCGAGCA GGCTAGCTACAACGA GACGAGCC | 12424 |
| 4364 | CUCGUCGU G CUCGCCAC | 3676 | GTGGCGAG GGCTAGCTACAACGA ACGACGAG | 12425 |
| 4368 | UCGUGCUC G CCACCGCU | 3677 | AGCGGTGG GGCTAGCTACAACGA GAGCACGA | 12426 |
| 4371 | UGCUCGCC A CCGCUACG | 3678 | CGTAGCGG GGCTAGCTACAACGA GGCGAGCA | 12427 |
| 4374 | UCGCCACC G CUACGCCU | 3679 | AGGCGTAG GGCTAGCTACAACGA GGTGGCGA | 12428 |
| 4377 | CCACCGCU A CGCCUCCG | 3680 | CGGAGGCG GGCTAGCTACAACGA AGCGGTGG | 12429 |
| 4379 | ACCGCUAC G CCUCCGGG | 3681 | CCCGGAGG GGCTAGCTACAACGA GTAGCGGT | 12430 |
| 4388 | CCUCCGGG A UCGGUCAC | 3682 | GTGACCGA GGCTAGCTACAACGA CCCGGAGG | 12431 |
| 4392 | CGGGAUCG G UCACCGUG | 3683 | CACGGTGA GGCTAGCTACAACGA CGATCCCG | 12432 |
| 4395 | GAUCGGUC A CCGUGCCA | 3684 | TGGCACGG GGCTAGCTACAACGA GACCGATC | 12433 |
| 4398 | CGGUCACC G UGCCACAU | 3685 | ATGTGGCA GGCTAGCTACAACGA GGTGACCG | 12434 |
| 4400 | GUCACCGU G CCACAUCC | 3686 | GGATGTGG GGCTAGCTACAACGA ACGGTGAC | 12435 |
| 4403 | ACCGUGCC A CAUCCCAA | 3687 | TTGGGATG GGCTAGCTACAACGA GGCACGGT | 12436 |
| 4405 | CGUGCCAC A UCCCAACA | 3688 | TGTTGGGA GGCTAGCTACAACGA GTGGCACG | 12437 |
| 4411 | ACAUCCCA A CAUCGAGG | 3689 | CCTCGATG GGCTAGCTACAACGA TGGGATGT | 12438 |
| 4413 | AUCCCAAC A UCGAGGAG | 3690 | CTCCTCGA GGCTAGCTACAACGA GTTGGGAT | 12439 |
| 4422 | UCGAGGAG A UAGCCUUG | 3691 | CAAGGCTA GGCTAGCTACAACGA CTCCTCGA | 12440 |
| 4425 | AGGAGAUA G CCUUGUCC | 3692 | GGACAAGG GGCTAGCTACAACGA TATCTCCT | 12441 |
| 4430 | AUAGCCUU G UCCAACAC | 3693 | GTGTTGGA GGCTAGCTACAACGA AAGGCTAT | 12442 |
| 4435 | CUUGUCCA A CACCGGAG | 3694 | CTCCGGTG GGCTAGCTACAACGA TGGACAAG | 12443 |
| 4437 | UGUCCAAC A CCGGAGAG | 3695 | CTCTCCGG GGCTAGCTACAACGA GTTGGACA | 12444 |
| 4446 | CCGGAGAG A UCCCCUUC | 3696 | GAAGGGGA GGCTAGCTACAACGA CTCTCCGG | 12445 |
| 4456 | CCCCUUCU A UGGCAAAG | 3697 | CTTTGCCA GGCTAGCTACAACGA AGAAGGGG | 12446 |
| 4459 | CUUCUAUG G CAAAGCCA | 3698 | TGGCTTTG GGCTAGCTACAACGA CATAGAAG | 12447 |
| 4464 | AUGGCAAA G CCAUCCCC | 3699 | GGGGATGG GGCTAGCTACAACGA TTTGCCAT | 12448 |
| 4467 | GCAAAGCC A UCCCCAUC | 3700 | GATGGGGA GGCTAGCTACAACGA GGCTTTGC | 12449 |
| 4473 | CCAUCCCC A UCGAGACC | 3701 | GGTCTCGA GGCTAGCTACAACGA GGGGATGG | 12450 |
| 4479 | CCAUCGAG A CCAUCAAA | 3702 | TTTGATGG GGCTAGCTACAACGA CTCGATGG | 12451 |
| 4482 | UCGAGACC A UCAAAGGG | 3703 | CCCTTTGA GGCTAGCTACAACGA GGTCTCGA | 12452 |
| 4496 | GGGGGGAG G CAUCUCAU | 3704 | ATGAGATG GGCTAGCTACAACGA CTCCCCCC | 12453 |
| 4498 | GGGGAGGC A UCUCAUCU | 3705 | AGATGAGA GGCTAGCTACAACGA GCCTCCCC | 12454 |
| 4503 | GGCAUCUC A UCUUCUGC | 3706 | GCAGAAGA GGCTAGCTACAACGA GAGATGCC | 12455 |
| 4510 | CAUCUUCU G CCAUUCCA | 3707 | TGGAATGG GGCTAGCTACAACGA AGAAGATG | 12456 |
| 4513 | CUUCUGCC A UUCCAAGA | 3708 | TCTTGGAA GGCTAGCTACAACGA GGCAGAAG | 12457 |
| 4526 | AAGAAGAA A UGUGACGA | 3709 | TCGTCACA GGCTAGCTACAACGA TTCTTCTT | 12458 |
| 4528 | GAAGAAAU G UGACGAGC | 3710 | GCTCGTCA GGCTAGCTACAACGA ATTTCTTC | 12459 |
| 4531 | GAAAUGUG A CGAGCUCG | 3711 | CGAGCTCG GGCTAGCTACAACGA CACATTTC | 12460 |
| 4535 | UGUGACGA G CUCGCUGC | 3712 | GCAGCGAG GGCTAGCTACAACGA TCGTCACA | 12461 |
| 4539 | ACGAGCUC G CUGCAAAG | 3713 | CTTTGCAG GGCTAGCTACAACGA GAGCTCGT | 12462 |
| 4542 | AGCUCGCU G CAAAGCUG | 3714 | CAGCTTTG GGCTAGCTACAACGA AGCGAGCT | 12463 |
| 4547 | GCUGCAAA G CUGUCGGG | 3715 | CCCGACAG GGCTAGCTACAACGA TTTGCAGC | 12464 |
| 4550 | GCAAAGCU G UCGGGCCU | 3716 | AGGCCCGA GGCTAGCTACAACGA AGCTTTGC | 12465 |
| 4555 | GCUGUCGG G CCUCGGAC | 3717 | GTCCGAGG GGCTAGCTACAACGA CCGACAGC | 12466 |
| 4562 | GGCCUCGG A CUUAACGC | 3718 | GCGTTAAG GGCTAGCTACAACGA CCGAGGCC | 12467 |
| 4567 | CGGACUUA A CGCUGUAG | 3719 | CTACAGCG GGCTAGCTACAACGA TAAGTCCG | 12468 |
| 4569 | GACUUAAC G CUGUAGCG | 3720 | CGCTACAG GGCTAGCTACAACGA GTTAAGTC | 12469 |
| 4572 | UUAACGCU G UAGCGUAU | 3721 | ATACGCTA GGCTAGCTACAACGA AGCGTTAA | 12470 |
| 4575 | ACGCUGUA G CGUAUUAC | 3722 | GTAATACG GGCTAGCTACAACGA TACAGCGT | 12471 |
| 4577 | GCUGUAGC G UAUUACCG | 3723 | CGGTAATA GGCTAGCTACAACGA GCTACAGC | 12472 |
| 4579 | UGUAGCGU A UUACCGGG | 3724 | CCCGGTAA GGCTAGCTACAACGA ACGCTACA | 12473 |
| 4582 | AGCGUAUU A CCGGGGUC | 3725 | GACCCCGG GGCTAGCTACAACGA AATACGCT | 12474 |
| 4588 | UUACCGGG G UCUCGACG | 3726 | CGTCGAGA GGCTAGCTACAACGA CCCGGTAA | 12475 |
| 4594 | GGGUCUCG A CGUGUCCG | 3727 | CGGACACG GGCTAGCTACAACGA CGAGACCC | 12476 |
| 4596 | GUCUCGAC G UGUCCGUC | 3728 | GACGGACA GGCTAGCTACAACGA GTCGAGAC | 12477 |
| 4598 | CUCGACGU G UCCGUCAU | 3729 | ATGACGGA GGCTAGCTACAACGA ACGTCGAG | 12478 |
| 4602 | ACGUGUCC G UCAUACCG | 3730 | CGGTATGA GGCTAGCTACAACGA GGACACGT | 12479 |
| 4605 | UGUCCGUC A UACCGGCC | 3731 | GGCCGGTA GGCTAGCTACAACGA GACGGACA | 12480 |
| 4607 | UCCGUCAU A CCGGCCAG | 3732 | CTGGCCGG GGCTAGCTACAACGA ATGACGGA | 12481 |
| 4611 | UCAUACCG G CCAGCGGG | 3733 | CCCGCTGG GGCTAGCTACAACGA CGGTATGA | 12482 |
| 4615 | ACCGGCCA G CGGGGACG | 3734 | CGTCCCCG GGCTAGCTACAACGA TGGCCGGT | 12483 |
| 4621 | CAGCGGGG A CGUCGUUG | 3735 | CAACGACG GGCTAGCTACAACGA CCCCGCTG | 12484 |
| 4623 | GCGGGGAC G UCGUUGUC | 3736 | GACAACGA GGCTAGCTACAACGA GTCCCCGC | 12485 |
| 4626 | GGGACGUC G UUGUCGUG | 3737 | CACGACAA GGCTAGCTACAACGA GACGTCCC | 12486 |
| 4629 | ACGUCGUU G UCGUGGCA | 3738 | TGCCACGA GGCTAGCTACAACGA AACGACGT | 12487 |
| 4632 | UCGUUGUC G UGGCAACA | 3739 | TGTTGCCA GGCTAGCTACAACGA GACAACGA | 12488 |
| 4635 | UUGUCGUG G CAACAGAC | 3740 | GTCTGTTG GGCTAGCTACAACGA CACGACAA | 12489 |
| 4638 | UCGUGGCA A CAGACGCU | 3741 | AGCGTCTG GGCTAGCTACAACGA TGCCACGA | 12490 |
| 4642 | GGCAACAG A CGCUCUAA | 3742 | TTAGAGCG GGCTAGCTACAACGA CTGTTGCC | 12491 |
| 4644 | CAACAGAC G CUCUAAUG | 3743 | CATTAGAG GGCTAGCTACAACGA GTCTGTTG | 12492 |
| 4650 | ACGCUCUA A UGACGGGC | 3744 | GCCCGTCA GGCTAGCTACAACGA TAGAGCGT | 12493 |
| 4653 | CUCUAAUG A CGGGCUAU | 3745 | ATAGCCCG GGCTAGCTACAACGA CATTAGAG | 12494 |
| 4657 | AAUGACGG G CUAUACCG | 3746 | CGGTATAG GGCTAGCTACAACGA CCGTCATT | 12495 |
| 4660 | GACGGGCU A UACCGGCG | 3747 | CGCCGGTA GGCTAGCTACAACGA AGCCCGTC | 12496 |
| 4662 | CGGGCUAU A CCGGCGAU | 3748 | ATCGCCGG GGCTAGCTACAACGA ATAGCCCG | 12497 |
| 4666 | CUAUACCG G CGAUUUUG | 3749 | CAAAATCG GGCTAGCTACAACGA CGGTATAG | 12498 |
| 4669 | UACCGGCG A UUUUGACU | 3750 | AGTCAAAA GGCTAGCTACAACGA CGCCGGTA | 12499 |
| 4675 | CGAUUUUG A CUCGGUGA | 3751 | TCACCGAG GGCTAGCTACAACGA CAAAATCG | 12500 |
| 4680 | UUGACUCG G UGAUCGAC | 3752 | GTCGATCA GGCTAGCTACAACGA CGAGTCAA | 12501 |
| 4683 | ACUCGGUG A UCGACUGU | 3753 | ACAGTCGA GGCTAGCTACAACGA CACCGAGT | 12502 |
| 4687 | GGUGAUCG A CUGUAAUA | 3754 | TATTACAG GGCTAGCTACAACGA CGATCACC | 12503 |
| 4690 | GAUCGACU G UAAUACAU | 3755 | ATGTATTA GGCTAGCTACAACGA AGTCGATC | 12504 |
| 4693 | CGACUGUA A UACAUGUG | 3756 | CACATGTA GGCTAGCTACAACGA TACAGTCG | 12505 |
| 4695 | ACUGUAAU A CAUGUGUC | 3757 | GACACATG GGCTAGCTACAACGA ATTACAGT | 12506 |
| 4697 | UGUAAUAC A UGUGUCAC | 3758 | GTGACACA GGCTAGCTACAACGA GTATTACA | 12507 |
| 4699 | UAAUACAU G UGUCACCC | 3759 | GGGTGACA GGCTAGCTACAACGA ATGTATTA | 12508 |
| 4701 | AUACAUGU G UCACCCAA | 3760 | TTGGGTGA GGCTAGCTACAACGA ACATGTAT | 12509 |
| 4704 | CAUGUGUC A CCCAAACA | 3761 | TGTTTCGG GCCTAGCTACAACGA GACACATG | 12510 |
| 4710 | UCACCCAA A CAGUCGAC | 3762 | GTCGACTG GGCTAGCTACAACGA TTGGGTGA | 12511 |
| 4713 | CCCAAACA G UCGACUUC | 3763 | GAAGTCGA GGCTAGCTACAACGA TGTTTGCG | 12512 |
| 4717 | AACAGUCG A CUUCAGCU | 3764 | AGCTGAAG GGCTAGCTACAACGA CGACTGTT | 12513 |
| 4723 | CGACUUCA G CUUGGACC | 3765 | GGTCCAAG GGCTAGCTACAACGA TGAAGTCG | 12514 |
| 4729 | CAGCUUGG A CCCUACCU | 3766 | AGGTAGGG GGCTAGCTACAACGA CCAAGCTG | 12515 |
| 4734 | UGGACCCU A CCUUCACC | 3767 | GGTGAAGG GGCTAGCTACAACGA AGGGTCCA | 12516 |
| 4740 | CUACCUUC A CCAUUGAG | 3768 | CTCAATGG GGCTAGCTACAACGA GAAGGTAG | 12517 |
| 4743 | CCUUCACC A UUGAGACG | 3769 | CGTCTCAA GGCTAGCTACAACGA GGTGAAGG | 12518 |
| 4749 | CCAUUGAG A CGACGACC | 3770 | GGTCGTCG GGCTAGCTACAACGA CTCAATGG | 12519 |
| 4752 | UUGAGACG A CGACCGUG | 3771 | CACGGTCG GGCTAGCTACAACGA CGTCTCAA | 12520 |
| 4755 | AGACGACG A CCGUGCCC | 3772 | GGGCACGG GGCTAGCTACAACGA CGTCGTCT | 12521 |
| 4758 | CGACGACC G UGCCCCAA | 3773 | TTGGGGCA GGCTAGCTACAACGA GGTCGTCG | 12522 |
| 4760 | ACGACCGU G CCCCAAGA | 3774 | TCTTGGGG GGCTAGCTACAACGA ACGGTCGT | 12523 |
| 4768 | GCCCCAAG A CGCAGUGU | 3775 | ACACTGCG GGCTAGCTACAACGA CTTGGGGC | 12524 |
| 4770 | CCCAAGAC G CAGUGUCC | 3776 | GGACACTG GGCTAGCTACAACGA GTCTTGGG | 12525 |
| 4773 | AAGACGCA G UGUCCCGC | 3777 | GCGGGACA GGCTAGCTACAACGA TGCGTCTT | 12526 |
| 4775 | GACGCAGU G UCCCGCUC | 3778 | GAGCGGGA GGCTAGCTACAACGA ACTGCGTC | 12527 |
| 4780 | AGUGUCCC G CUCGCAGA | 3779 | TCTGCGAG GGCTAGCTACAACGA GGGACACT | 12528 |
| 4784 | UCCCGCUC G CAGAGGCG | 3780 | CGCCTCTG GGCTAGCTACAACGA GAGCGGGA | 12529 |
| 4790 | UCGCAGAG G CGAGGUAG | 3781 | CTACCTCG GGCTAGCTACAACGA CTCTGCGA | 12530 |
| 4795 | GAGGCGAG G UAGGACCG | 3782 | CGGTCCTA GGCTAGCTACAACGA CTCGCCTC | 12531 |
| 4800 | GAGGUAGG A CCGGUAGG | 3783 | CCTACCGG GGCTAGCTACAACGA CCTACCTC | 12532 |
| 4804 | UAGGACCG G UAGGGGCA | 3784 | TGCCCCTA GGCTAGCTACAACGA CGGTCCTA | 12533 |
| 4810 | CGGUAGGG G CAGGAGAG | 3785 | CTCTCCTG GGCTAGCTACAACGA CCCTACCG | 12534 |
| 4819 | CAGGAGAG G CAUAUACA | 3786 | TGTATATG GGCTAGCTACAACGA CTCTCCTG | 12535 |
| 4821 | GGAGAGGC A UAUACAGG | 3787 | CCTGTATA GGCTAGCTACAACGA GCCTCTCC | 12536 |
| 4823 | AGAGGCAU A UACAGGUU | 3788 | AACCTGTA GGCTAGCTACAACGA ATGCCTCT | 12537 |
| 4825 | AGGCAUAU A CAGGUUUG | 3789 | CAAACCTG GGCTAGCTACAACGA ATATGCCT | 12538 |
| 4829 | AUAUACAG G UUUGUGAC | 3790 | GTCACAAA GGCTAGCTACAACGA CTGTATAT | 12539 |
| 4833 | ACAGGUUU G UGACUCCA | 3791 | TGGAGTCA GGCTAGCTACAACGA AAACCTGT | 12540 |
| 4836 | GGUUUGUG A CUCCAGGA | 3792 | TCCTGGAG GGCTAGCTACAACGA CACAAACC | 12541 |
| 4847 | CCAGGAGA G CGGCCUUC | 3793 | GAAGGCCG GGCTAGCTACAACGA TCTCCTGG | 12542 |
| 4850 | GGAGAGCG G CCUUCGGG | 3794 | CCCGAAGG GGCTAGCTACAACGA CGCTCTCC | 12543 |
| 4858 | GCCUUCGG G CAUGUUCG | 3795 | CGAACATG GGCTAGCTACAACGA CCGAAGGC | 12544 |
| 4860 | CUUCGGGC A UGUUCGAC | 3796 | GTCGAACA GGCTAGCTACAACGA GCCCGAAG | 12545 |
| 4862 | UCGGGCAU G UUCGACUC | 3797 | GAGTCGAA GGCTAGCTACAACGA ATGCCCGA | 12546 |
| 4867 | CAUGUUCG A CUCCUCGG | 3798 | CCGAGGAG GGCTAGCTACAACGA CGAACATG | 12547 |
| 4875 | ACUCCUCG G UCCUGUGU | 3799 | ACACAGGA GGCTAGCTACAACGA CGAGGAGT | 12548 |
| 4880 | UCGGUCCU G UGUGAGUG | 3800 | CACTCACA GGCTAGCTACAACGA AGGACCGA | 12549 |
| 4882 | GGUCCUGU G UGAGUGCU | 3801 | AGCACTCA GGCTAGCTACAACGA ACAGGACC | 12550 |
| 4886 | CUGUGUGA G UGCUAUGA | 3802 | TCATAGCA GGCTAGCTACAACGA TCACACAG | 12551 |
| 4888 | GUGUGAGU G CUAUGACG | 3803 | CGTCATAG GGCTAGCTACAACGA ACTCACAC | 12552 |
| 4891 | UGAGUGCU A UGACGCGG | 3804 | CCGCGTCA GGCTAGCTACAACGA AGCACTCA | 12553 |
| 4894 | GUGCUAUG A CGCGGGAU | 3805 | ATCCCGCG GGCTAGCTACAACGA CATAGCAC | 12554 |
| 4896 | GCUAUGAC G CGGGAUGU | 3806 | ACATCCCG GGCTAGCTACAACGA GTCATAGC | 12555 |
| 4901 | GACGCGGG A UGUGCUUG | 3807 | CAAGCACA GGCTAGCTACAACGA CCCGCGTC | 12556 |
| 4903 | CGCGGGAU G UGCUUGGU | 3808 | ACCAAGCA GGCTAGCTACAACGA ATCCCGCG | 12557 |
| 4905 | CGGGAUGU G CUUGGUAC | 3809 | GTACCAAG GGCTAGCTACAACGA ACATCCCG | 12558 |
| 4910 | UGUGCUUG G UACGAGCU | 3810 | AGCTCGTA GGCTAGCTACAACGA CAAGCACA | 12559 |
| 4912 | UGCUUGGU A CGAGCUCA | 3811 | TGAGCTCG GGCTAGCTACAACGA ACCAAGCA | 12560 |
| 4916 | UGGUACGA G CUCACGCC | 3812 | GGCGTGAG GGCTAGCTACAACGA TCGTACCA | 12561 |
| 4920 | ACGAGCUC A CGCCCGCC | 3813 | GGCGGGCG GGCTAGCTACAACGA GAGCTCGT | 12562 |
| 4922 | GAGCUCAC G CCCGCCGA | 3814 | TCGGCGGG GGCTAGCTACAACGA GTGAGCTC | 12563 |
| 4926 | UCACGCCC G CCGAGACC | 3815 | GGTCTCGG GGCTAGCTACAACGA GGGCGTGA | 12564 |
| 4932 | CCGCCGAG A CCUCCGUU | 3816 | AACGGAGG GGCTAGCTACAACGA CTCGGCGG | 12565 |
| 4938 | AGACCUCC G UUAGGUUG | 3817 | CAACCTAA GGCTAGCTACAACGA GGAGGTCT | 12566 |
| 4943 | UCCGUUAG G UUGCGGGC | 3818 | GCCCGCAA GGCTAGCTACAACGA CTAACGGA | 12567 |
| 4946 | GUUAGGUU G CGGGCUUA | 3819 | TAAGCCCG GGCTAGCTACAACGA AACCTAAC | 12568 |
| 4950 | GGUUGCGG G CUUACCUA | 3820 | TAGGTAAG GGCTAGCTACAACGA CCGCAACC | 12569 |
| 4954 | GCGGGCUU A CCUAAAUA | 3821 | TATTTAGG GGCTAGCTACAACGA AAGCCCGC | 12570 |
| 4960 | UUACCUAA A UACACCAG | 3822 | CTGGTGTA GGCTAGCTACAACGA TTAGGTAA | 12571 |
| 4962 | ACCUAAAU A CACCAGGG | 3823 | CCCTGGTG GGCTAGCTACAACGA ATTTAGGT | 12572 |
| 4964 | CUAAAUAC A CCAGGGUU | 3824 | AACCCTGG GGCTAGCTACAACGA GTATTTAG | 12573 |
| 4970 | ACACCAGG G UUGCCCUU | 3825 | AAGGGCAA GGCTAGCTACAACGA CCTGGTGT | 12574 |
| 4973 | CCAGGGUU G CCCUUCUG | 3826 | CAGAAGGG GGCTAGCTACAACGA AACCCTGG | 12575 |
| 4981 | GCCCUUCU G CCAGGACC | 3827 | GGTCCTGG GGCTAGCTACAACGA AGAAGGGC | 12576 |
| 4987 | CUGCCAGG A CCAUCUGG | 3828 | CCAGATGG GGCTAGCTACAACGA CCTGGCAG | 12577 |
| 4990 | CCAGGACC A UCUGGAGU | 3829 | ACTCCAGA GGCTAGCTACAACGA GGTCCTGG | 12578 |
| 4997 | CAUCUGGA G UUCUGGGA | 3830 | TCCCAGAA GGCTAGCTACAACGA TCCAGATG | 12579 |
| 5008 | CUGGGAGG G UGUCUUCA | 3831 | TGAAGACA GGCTAGCTACAACGA CCTCCCAG | 12580 |
| 5010 | GGGAGGGU G UCUUCACA | 3832 | TGTGAAGA GGCTAGCTACAACGA ACCCTCCC | 12581 |
| 5016 | GUGUCUUC A CAGGCCUC | 3833 | GAGGCCTG GGCTAGCTACAACGA GAAGACAC | 12582 |
| 5020 | CUUCACAG G CCUCACCC | 3834 | GGGTGAGG GGCTAGCTACAACGA CTGTGAAG | 12583 |
| 5025 | CAGGCCUC A CCCACAUA | 3835 | TATGTGGG GGCTAGCTACAACGA GAGGCCTG | 12584 |
| 5029 | CCUCACCC A CAUAGAUG | 3836 | CATCTATG GGCTAGCTACAACGA GGGTGAGG | 12585 |
| 5031 | UCACCCAC A UAGAUGCC | 3837 | GGCATCTA GGCTAGCTACAACGA GTGGGTGA | 12586 |
| 5035 | CCACAUAG A UGCCCACU | 3838 | AGTGGGCA GGCTAGCTACAACGA CTATGTGG | 12587 |
| 5037 | ACAUAGAU G CCCACUUC | 3839 | GAAGTGGG GGCTAGCTACAACGA ATCTATGT | 12588 |
| 5041 | AGAUGCCC A CUUCUUGU | 3840 | ACAAGAAG GGCTAGCTACAACGA GGGCATCT | 12589 |
| 5048 | CACUUCUU G UCCCAGAC | 3841 | GTCTGGGA GGCTAGCTACAACGA AAGAAGTG | 12590 |
| 5055 | UGUCCCAG A CCAAGCAG | 3842 | CTGCTTGG GGCTAGCTACAACGA CTGGGACA | 12591 |
| 5060 | CAGACCAA G CAGGCAGG | 3843 | CCTGCCTG GGCTAGCTACAACGA TTGGTCTG | 12592 |
| 5064 | CCAAGCAG G CAGGAGAA | 3844 | TTCTCCTG GGCTAGCTACAACGA CTGCTTGG | 12593 |
| 5074 | AGGAGAAA A CCUCCCCU | 3845 | AGGGGAGG GGCTAGCTACAACGA TTTCTCCT | 12594 |
| 5083 | CCUCCCCU A CCUGGUAG | 3846 | CTACCAGG GGCTAGCTACAACGA AGGGGAGG | 12595 |
| 5088 | CCUACCUG G UAGCAUAC | 3847 | GTATGCTA GGCTAGCTACAACGA CAGGTAGG | 12596 |
| 5091 | ACCUGGUA G CAUACCAA | 3848 | TTGGTATG GGCTAGCTACAACGA TACCAGGT | 12597 |
| 5093 | CUGGUAGC A UACCAAGC | 3849 | GCTTGGTA GGCTAGCTACAACGA GCTACCAG | 12598 |
| 5095 | GGUAGCAU A CCAAGCCA | 3850 | TGGCTTGG GGCTAGCTACAACGA ATGCTACC | 12599 |
| 5100 | CAUACCAA G CCACAGUG | 3851 | CACTGTGG GGCTAGCTACAACGA TTGGTATG | 12600 |
| 5103 | ACCAAGCC A CAGUGUGC | 3852 | GCACACTG GGCTAGCTACAACGA GGCTTGGT | 12601 |
| 5106 | AAGCCACA G UGUGCGCC | 3853 | GGCGCACA GGCTAGCTACAACGA TGTGGCTT | 12602 |
| 5108 | GCCACAGU G UGCGCCAG | 3854 | CTGGCGCA GGCTAGCTACAACGA ACTGTGGC | 12603 |
| 5110 | CACAGUGU G CGCCAGGG | 3855 | CCCTGGCG GGCTAGCTACAACGA ACACTGTG | 12604 |
| 5112 | CAGUGUGC G CCAGGGCU | 3856 | AGCCCTGG GGCTAGCTACAACGA GCACACTG | 12605 |
| 5118 | GCGCCAGG G CUCAGGCU | 3857 | AGCCTGAG GGCTAGCTACAACGA CCTGGCGC | 12606 |
| 5124 | GGGCUCAG G CUCCACCC | 3858 | GGGTGGAG GGCTAGCTACAACGA CTGAGCCC | 12607 |
| 5129 | CAGGCUCC A CCCCCAUC | 3859 | GATGGGGG GGCTAGCTACAACGA GGAGCCTG | 12608 |
| 5135 | CCACCCCC A UCGUGGGA | 3860 | TCCCACGA GGCTAGCTACAACGA GGGGGTGG | 12609 |
| 5138 | CCCCCAUC G UGGGAUCA | 3861 | TGATCCCA GGCTAGCTACAACGA GATGGGGG | 12610 |
| 5143 | AUCGUGGG A UCAAAUGU | 3862 | ACATTTGA GGCTAGCTACAACGA CCCACGAT | 12611 |
| 5148 | GGGAUCAA A UGUGGAAG | 3863 | CTTCCACA GGCTAGCTACAACGA TTGATCCC | 12612 |
| 5150 | GAUCAAAU G UGGAAGUG | 3864 | CACTTCCA GGCTAGCTACAACGA ATTTGATC | 12613 |
| 5156 | AUGUGGAA G UGUCUCAC | 3865 | GTGAGACA GGCTAGCTACAACGA TTCCACAT | 12614 |
| 5158 | GUGGAAGU G UCUCACAC | 3866 | GTGTGAGA GGCTAGCTACAACGA ACTTCCAC | 12615 |
| 5163 | AGUGUCUC A CACGGCUA | 3867 | TAGCCGTG GGCTAGCTACAACGA GAGACACT | 12616 |
| 5165 | UGUCUCAC A CGGCUAAA | 3868 | TTTAGCCG GGCTAGCTACAACGA GTGAGACA | 12617 |
| 5168 | CUCACACG G CUAAAGCC | 3869 | GGCTTTAG GGCTAGCTACAACGA CGTGTGAG | 12618 |
| 5174 | CGGCUAAA G CCUACGCU | 3870 | AGCGTAGG GGCTAGCTACAACGA TTTAGCCG | 12619 |
| 5178 | UAAAGCCU A CGCUACAC | 3871 | GTGTAGCG GGCTAGCTACAACGA AGGCTTTA | 12620 |
| 5180 | AAGCCUAC G CUACACGG | 3872 | CCGTGTAG GGCTAGCTACAACGA GTAGGCTT | 12621 |
| 5183 | CCUACGCU A CACGGGCC | 3873 | GGCCCGTG GGCTAGCTACAACGA AGCGTAGG | 12622 |
| 5185 | UACGCUAC A CGGGCCAA | 3874 | TTGGCCCG GGCTAGCTACAACGA GTAGCGTA | 12623 |
| 5189 | CUACACGG G CCAACACC | 3875 | GGTGTTGG GGCTAGCTACAACGA CCGTGTAG | 12624 |
| 5193 | ACGGGCCA A CACCCCUG | 3876 | CAGGGGTG GGCTAGCTACAACGA TGGCCCGT | 12625 |
| 5195 | GGGCCAAC A CCCCUGCU | 3877 | AGCAGGGG GGCTAGCTACAACGA GTTGGCCC | 12626 |
| 5201 | ACACCCCU G CUGUAUAG | 3878 | CTATACAC GGCTACCTACAACGA AGGGGTGT | 12627 |
| 5204 | CCCCUGCU G UAUAGGCU | 3879 | AGCCTATA GGCTAGCTACAACGA AGCAGGGG | 12628 |
| 5206 | CCUGCUGU A UAGGCUAG | 3880 | CTAGCCTA GGCTAGCTACAACCA ACAGCAGG | 12629 |
| 5210 | CUGUAUAG G CUAGGAGC | 3881 | GCTCCTAG GGCTAGCTACAACGA CTATACAG | 12630 |
| 5217 | GGCUAGGA G CCGUCCAA | 3882 | TTGGACGG GGCTAGCTACAACGA TCCTAGCC | 12631 |
| 5220 | UAGGAGCC G UCCAAAAU | 3883 | ATTTTGGA GGCTAGCTACAACGA GGCTCCTA | 12632 |
| 5227 | CGUCCAAA A UGAUGUCA | 3884 | TGACATCA GGCTAGCTACAACGA TTTGGACG | 12633 |
| 5230 | CCAAAAUG A UGUCACCC | 3885 | GGGTGACA GGCTAGCTACAACGA CATTTTGG | 12634 |
| 5232 | AAAAUGAU G UCACCCUC | 3886 | GAGGGTGA GGCTAGCTACAACGA ATCATTTT | 12635 |
| 5235 | AUGAUGUC A CCCUCACA | 3887 | TGTGAGGG GGCTAGCTACAACGA GACATCAT | 12636 |
| 5241 | UCACCCUC A CACACCCC | 3888 | GGGGTGTG GGCTAGCTACAACGA GAGGGTGA | 12637 |
| 5243 | ACCCUCAC A CACCCCAU | 3889 | ATGGGGTG GGCTAGCTACAACGA GTGAGGGT | 12638 |
| 5245 | CCUCACAC A CCCCAUAA | 3890 | TTATGGGG GGCTAGCTACAACGA GTGTGAGG | 12639 |
| 5250 | CACACCCC A UAACCAAA | 3891 | TTTGGTTA GGCTAGCTACAACGA GGGGTGTG | 12640 |
| 5253 | ACCCCAUA A CCAAAUAC | 3892 | GTATTTGG GGCTAGCTACAACGA TATGGGGT | 12641 |
| 5258 | AUAACCAA A UACAUCAU | 3893 | ATGATGTA GGCTAGCTACAACGA TTGGTTAT | 12642 |
| 5260 | AACCAAAU A CAUCAUCA | 3894 | TCATGATG GGCTAGCTACAACGA ATTTGGTT | 12643 |
| 5262 | CCAAAUAC A UCAUGACA | 3895 | TGTCATGA GGCTAGCTACAACGA GTATTTGG | 12644 |
| 5265 | AAUACAUC A UGACAUGC | 3896 | GCATGTCA GGCTAGCTACAACGA GATGTATT | 12645 |
| 5268 | ACAUCAUG A CAUGCAUG | 3897 | CATGCATG GGCTAGCTACAACGA CATGATGT | 12646 |
| 5270 | AUCAUGAC A UGCAUGUC | 3898 | GACATGCA GGCTAGCTACAACGA GTCATGAT | 12647 |
| 5272 | CAUGACAU G CAUGUCGG | 3899 | CCGACATG GGCTAGCTACAACGA ATGTCATG | 12648 |
| 5274 | UGACAUGC A UGUCGGCU | 3900 | AGCCGACA GGCTAGCTACAACGA GCATGTCA | 12649 |
| 5276 | ACAUGCAU G UCGGCUGA | 3901 | TCAGCCGA GGCTAGCTACAACGA ATGCATGT | 12650 |
| 5280 | GCAUGUCG G CUGACCUG | 3902 | CAGGTCAG GGCTAGCTACAACGA CGACATGC | 12651 |
| 5284 | GUCGGCUG A CCUGGAGG | 3903 | CCTCCAGG GGCTAGCTACAACGA CAGCCGAC | 12652 |
| 5292 | ACCUGGAG G UCGUCACC | 3904 | GGTGACGA GGCTAGCTACAACGA CTCCAGGT | 12653 |
| 5295 | UGGAGGUC G UCACCAGC | 3905 | GCTGGTGA GGCTAGCTACAACGA GACCTCCA | 12654 |
| 5298 | AGGUCGUC A CCAGCACC | 3906 | GGTGCTGG GGCTAGCTACAACGA GACGACCT | 12655 |
| 5302 | CGUCACCA G CACCUGGG | 3907 | CCCAGGTG GGCTAGCTACAACGA TGGTGACG | 12656 |
| 5304 | UCACCAGC A CCUGGGUG | 3908 | CACCCAGG GGCTAGCTACAACGA GCTGGTGA | 12657 |
| 5310 | GCACCUGG G UGCUAGUA | 3909 | TACTAGCA GGCTAGCTACAACGA CCAGGTGC | 12658 |
| 5312 | ACCUGGGU G CUAGUAGG | 3910 | CCTACTAG GGCTAGCTACAACGA ACCCAGGT | 12659 |
| 5316 | GGGUGCUA G UAGGUGGC | 3911 | GCCACCTA GGCTAGCTACAACGA TAGCACCC | 12660 |
| 5320 | GCUAGUAG G UGGCGUCC | 3912 | GGACGCCA GGCTAGCTACAACGA CTACTAGC | 12661 |
| 5323 | AGUAGGUG G CGUCCUGG | 3913 | CCAGGACG GGCTAGCTACAACGA CACCTACT | 12662 |
| 5325 | UAGGUGGC G UCCUGGCA | 3914 | TGCCAGGA GGCTAGCTACAACGA GCCACCTA | 12663 |
| 5331 | GCGUCCUG G CAGCUCUG | 3915 | CAGAGCTG GGCTAGCTACAACGA CAGGACGC | 12664 |
| 5334 | UCCUGGCA G CUCUGACC | 3916 | GGTCAGAG GGCTAGCTACAACGA TGCCAGGA | 12665 |
| 5340 | CAGCUCUG A CCGCGUAU | 3917 | ATACGCGG GGCTAGCTACAACGA CAGAGCTG | 12666 |
| 5343 | CUCUGACC G CGUAUUGC | 3918 | GCAATACG GGCTAGCTACAACGA GGTCAGAG | 12667 |
| 5345 | CUGACCGC G UAUUGCCU | 3919 | AGGCAATA GGCTAGCTACAACGA GCGGTCAG | 12668 |
| 5347 | GACCGCGU A UUGCCUGA | 3920 | TCAGGCAA GGCTAGCTACAACGA ACGCGGTC | 12669 |
| 5350 | CGCGUAUU G CCUGACGA | 3921 | TCGTCAGG GGCTAGCTACAACGA AATACGCG | 12670 |
| 5355 | AUUGCCUG A CGACAGGC | 3922 | GCCTGTCG GGCTAGCTACAACGA CAGGCAAT | 12671 |
| 5358 | GCCUGACG A CAGGCAGC | 3923 | GCTGCCTG GGCTAGCTACAACGA CGTCAGGC | 12672 |
| 5362 | GACGACAG G CAGCGUGG | 3924 | CCACGCTG GGCTAGCTACAACGA CTGTCGTC | 12673 |
| 5365 | GACAGGCA G CGUGGUCA | 3925 | TGACCACG GGCTAGCTACAACGA TGCCTGTC | 12674 |
| 5367 | CAGGCAGC G UGGUCAUU | 3926 | AATGACCA GGCTAGCTACAACGA GCTGCCTG | 12675 |
| 5370 | GCAGCGUG G UCAUUGUG | 3927 | CACAATGA GGCTAGCTACAACGA CACGCTGC | 12676 |
| 5373 | GCGUGGUC A UUGUGGGC | 3928 | GCCCACAA GGCTAGCTACAACGA GACCACGC | 12677 |
| 5376 | UGGUCAUU G UGGGCAGA | 3929 | TCTGCCCA GGCTAGCTACAACGA AATGACCA | 12678 |
| 5380 | CAUUGUGG G CAGAAUCA | 3930 | TGATTCTG GGCTAGCTACAACGA CCACAATG | 12679 |
| 5385 | UGGGCAGA A UCAUCUUG | 3931 | CAAGATGA GGCTAGCTACAACGA TCTGCCCA | 12680 |
| 5388 | GCAGAAUC A UCUUGUCC | 3932 | GGACAAGA GGCTAGCTACAACGA GATTCTGC | 12681 |
| 5393 | AUCAUCUU G UCCGGGAA | 3933 | TTCCCGGA GGCTAGCTACAACGA AAGATGAT | 12682 |
| 5402 | UCCGGGAA G CCGGCUGU | 3934 | ACAGCCGG GGCTAGCTACAACGA TTCCCGGA | 12683 |
| 5406 | GGAAGCCG G CUGUUAUC | 3935 | GATAACAG GGCTAGCTACAACGA CGGCTTCC | 12684 |
| 5409 | AGCCGGCU G UUAUCCCC | 3936 | GGGGATAA GGCTAGCTACAACGA AGCCGGCT | 12685 |
| 5412 | CGGCUGUU A UCCCCGAC | 3937 | GTCGGGGA GGCTAGCTACAACGA AACAGCCG | 12686 |
| 5419 | UAUCCCCG A CAGGGAGG | 3938 | CCTCCCTG GGCTAGCTACAACGA CGGGGATA | 12687 |
| 5427 | ACAGGGAG G CUCUCUAC | 3939 | GTAGAGAG GGCTAGCTACAACGA CTCCCTGT | 12688 |
| 5434 | GGCUCUCU A CCAGGAGU | 3940 | ACTCCTGG GGCTAGCTACAACGA AGAGAGCC | 12689 |
| 5441 | UACCAGGA G UUCGAUGA | 3941 | TCATCGAA GGCTAGCTACAACGA TCCTGGTA | 12690 |
| 5446 | GGAGUUCG A UGAGAUGG | 3942 | CCATCTCA GGCTAGCTACAACGA CGAACTCC | 12691 |
| 5451 | UCGAUGAG A UGGAGGAG | 3943 | CTCCTCCA GGCTAGCTACAACGA CTCATCGA | 12692 |
| 5459 | AUGGAGGA G UGUGCCUC | 3944 | GAGGCACA GGCTAGCTACAACGA TCCTCCAT | 12693 |
| 5461 | GGAGGAGU G UGCCUCAC | 3945 | GTGAGGCA GGCTAGCTACAACGA ACTCCTCC | 12694 |
| 5463 | AGGAGUGU G CCUCACAC | 3946 | GTGTGAGG GGCTAGCTACAACGA ACACTCCT | 12695 |
| 5468 | UGUGCCUC A CACCUCCC | 3947 | GGGAGGTG GGCTAGCTACAACGA GAGGCACA | 12696 |
| 5470 | UGCCUCAC A CCUCCCUU | 3948 | AAGGGAGG GGCTAGCTACAACGA GTGAGGCA | 12697 |
| 5479 | CCUCCCUU A CAUCGAAC | 3949 | GTTCGATG GGCTAGCTACAACGA AAGGGAGG | 12698 |
| 5481 | UCCCUUAC A UCGAACAG | 3950 | CTGTTCGA GGCTAGCTACAACGA GTAAGGGA | 12699 |
| 5486 | UACAUCGA A CAGGGGAU | 3951 | ATCCCCTG GGCTAGCTACAACGA TCGATGTA | 12700 |
| 5493 | AACAGGGG A UGCAGCUC | 3952 | GAGCTGCA GGCTAGCTACAACGA CCCCTGTT | 12701 |
| 5495 | CAGGGGAU G CAGCUCGC | 3953 | GCGAGCTG GGCTAGCTACAACGA ATCCCCTG | 12702 |
| 5498 | GGGAUGCA G CUCGCCGA | 3954 | TCGGCGAG GGCTAGCTACAACGA TGCATCCC | 12703 |
| 5502 | UGCAGCUC G CCGAGCAG | 3955 | CTGCTCGG GGCTAGCTACAACGA GAGCTGCA | 12704 |
| 5507 | CUCGCCGA G CAGUUCAA | 3956 | TTGAACTG GGCTAGCTACAACGA TCGGCGAG | 12705 |
| 5510 | GCCGAGCA G UUCAAGCA | 3957 | TGCTTGAA GGCTAGCTACAACGA TGCTCGGC | 12706 |
| 5516 | CAGUUCAA G CAGAAGGC | 3958 | GCCTTCTG GGCTAGCTACAACGA TTGAACTG | 12707 |
| 5523 | AGCAGAAG G CGCUCGGA | 3959 | TCCGAGCG GGCTAGCTACAACGA CTTCTGCT | 12708 |
| 5525 | CAGAAGGC G CUCGGAUU | 3960 | AATCCGAG GGCTAGCTACAACGA GCCTTCTG | 12709 |
| 5531 | GCGCUCGG A UUGCUGCA | 3961 | TGCAGCAA GGCTAGCTACAACGA CCGAGCGC | 12710 |
| 5534 | CUCGGAUU G CUGCAAAC | 3962 | GTTTGCAG GGCTAGCTACAACGA AATCCGAG | 12711 |
| 5537 | GGAUUGCU G CAAACAGC | 3963 | GCTGTTTG GGCTAGCTACAACGA AGCAATCC | 12712 |
| 5541 | UGCUGCAA A CAGCCACC | 3964 | GGTGGCTG GGCTAGCTACAACGA TTGCAGCA | 12713 |
| 5544 | UGCAAACA G CCACCAAC | 3965 | GTTGGTGG GGCTAGCTACAACGA TGTTTGCA | 12714 |
| 5547 | AAACAGCC A CCAACCAA | 3966 | TTGGTTGG GGCTAGCTACAACGA GGCTGTTT | 12715 |
| 5551 | AGCCACCA A CCAAGCGG | 3967 | CCGCTTGG GGCTAGCTACAACGA TGGTGGCT | 12716 |
| 5556 | CCAACCAA G CGGAGGCU | 3968 | AGCCTCCG GGCTAGCTACAACGA TTGGTTGG | 12717 |
| 5562 | AAGCGGAG G CUGCUGCU | 3969 | AGCAGCAG GGCTAGCTACAACGA CTCCGCTT | 12718 |
| 5565 | CGGAGGCU G CUGCUCCC | 3970 | GGGAGCAG GGCTAGCTACAACGA AGCCTCCG | 12719 |
| 5568 | AGGCUGCU G CUCCCGUG | 3971 | CACGGGAG GGCTAGCTACAACGA AGCAGCCT | 12720 |
| 5574 | CUGCUCCC G UGGUGGAA | 3972 | TTCCACCA GGCTAGCTACAACGA GGGAGCAG | 12721 |
| 5577 | CUCCCGUG G UGGAAUCC | 3973 | GGATTCCA GGCTAGCTACAACGA CACGGGAG | 12722 |
| 5582 | GUGGUGGA A UCCAAGUG | 3974 | CACTTGGA GGCTAGCTACAACGA TCCACCAC | 12723 |
| 5588 | GAAUCCAA G UGGCGAGC | 3975 | GCTCGCCA GGCTAGCTACAACGA TTGGATTC | 12724 |
| 5591 | UCCAAGUG G CGAGCCCU | 3976 | AGGGCTCG GGCTAGCTACAACGA CACTTGGA | 12725 |
| 5595 | AGUGGCGA G CCCUUGAG | 3977 | CTCAAGGG GGCTAGCTACAACGA TCGCCACT | 12726 |
| 5604 | CCCUUGAG G CUUUCUGG | 3978 | CCAGAAAG GGCTAGCTACAACGA CTCAAGGG | 12727 |
| 5613 | CUUUCUGG G CGAAGCAC | 3979 | GTGCTTCG GGCTAGCTACAACGA CCAGAAAG | 12728 |
| 5618 | UGGGCGAA G CACAUGUG | 3980 | CACATGTG GGCTAGCTACAACGA TTCGCCCA | 12729 |
| 5620 | GGCGAAGC A CAUGUGGA | 3981 | TCCACATG GGCTAGCTACAACGA GCTTCGCC | 12730 |
| 5622 | CGAAGCAC A UGUGGAAU | 3982 | ATTCCACA GGCTAGCTACAACGA GTGCTTCG | 12731 |
| 5624 | AAGCACAU G UGGAAUUU | 3983 | AAATTCCA GGCTAGCTACAACGA ATGTGCTT | 12732 |
| 5629 | CAUGUGGA A UUUCAUCA | 3984 | TGATGAAA GGCTAGCTACAACGA TCCACATG | 12733 |
| 5634 | GGAAUUUC A UCAGCGGG | 3985 | CCCGCTGA GGCTAGCTACAACGA GAAATTCC | 12734 |
| 5638 | UUUCAUCA G CGGGAUAC | 3986 | GTATCCCG GGCTAGCTACAACGA TGATGAAA | 12735 |
| 5643 | UCAGCGGG A UACAGUAC | 3987 | GTACTGTA GGCTAGCTACAACGA CCCGCTGA | 12736 |
| 5645 | AGCGGGAU A CAGUACCU | 3988 | AGGTACTG GGCTAGCTACAACGA ATCCCGCT | 12737 |
| 5648 | GGGAUACA G UACCUAGC | 3989 | GCTAGGTA GGCTAGCTACAACGA TGTATCCC | 12738 |
| 5650 | GAUACAGU A CCUAGCAG | 3990 | CTGCTAGG GGCTAGCTACAACGA ACTGTATC | 12739 |
| 5655 | AGUACCUA G CAGGCUUG | 3991 | CAAGCCTG GGCTAGCTACAACGA TAGGTACT | 12740 |
| 5659 | CCUAGCAG G CUUGUCCA | 3992 | TGGACAAG GGCTAGCTACAACGA CTGCTAGG | 12741 |
| 5663 | GCAGGCUU G UCCACUCU | 3993 | AGAGTGGA GGCTAGCTACAACGA AAGCCTGC | 12742 |
| 5667 | GCUUGUCC A CUCUGCCU | 3994 | AGGCAGAG GGCTAGCTACAACGA GGACAAGC | 12743 |
| 5672 | UCCACUCU G CCUGGGAA | 3995 | TTCCCAGG GGCTAGCTACAACGA AGAGTGGA | 12744 |
| 5680 | GCCUGGGA A CCCCGCGA | 3996 | TCGCGGGG GGCTAGCTACAACGA TCCCAGOC | 12745 |
| 5685 | GGAACCCC G CGAUAGCA | 3997 | TGCTATCG GGCTAGCTACAACGA GGGGTTCC | 12746 |
| 5688 | ACCCCGCG A UAGCAUCA | 3998 | TGATGCTA GGCTAGCTACAACGA CGCGGGGT | 12747 |
| 5691 | CCGCGAUA G CAUCAUUG | 3999 | CAATGATG GGCTAGCTACAACGA TATCGCGG | 12748 |
| 5693 | GCGAUAGC A UCAUUGAU | 4000 | ATCAATGA GGCTAGCTACAACGA GCTATCGC | 12749 |
| 5696 | AUAGCAUC A UUGAUGGC | 4001 | GCCATCAA GGCTAGCTACAACGA GATGCTAT | 12750 |
| 5700 | CAUCAUUG A UGGCAUUC | 4002 | GAATGCCA GGCTAGCTACAACGA CAATGATG | 12751 |
| 5703 | CAUUGAUG G CAUUCACA | 4003 | TGTGAATG GGCTAGCTACAACGA CATCAATG | 12752 |
| 5705 | UUGAUGGC A UUCACAGC | 4004 | GCTGTGAA GGCTAGCTACAACGA GCCATCAA | 12753 |
| 5709 | UGGCAUUC A CAGCCUCC | 4005 | GGAGGCTG GGCTAGCTACAACGA GAATGCCA | 12754 |
| 5712 | CAUUCACA G CCUCCAUC | 4006 | GATGGAGG GGCTAGCTACAACGA TGTGAATG | 12755 |
| 5718 | CAGCCUCC A UCACCAGC | 4007 | GCTGGTGA GGCTAGCTACAACGA GGAGGCTG | 12756 |
| 5721 | CCUCCAUC A CCAGCCCG | 4008 | CGGGCTGG GGCTAGCTACAACGA GATGGAGG | 12757 |
| 5725 | CAUCACCA G CCCGCUCA | 4009 | TGAGCGGG GGCTAGCTACAACGA TGGTGATG | 12758 |
| 5729 | ACCAGCCC G CUCACCAC | 4010 | GTGGTGAG GGCTAGCTACAACGA GGGCTGGT | 12759 |
| 5733 | GCCCGCUC A CCACCCAA | 4011 | TTGGGTGG GGCTAGCTACAACGA GAGCGGGC | 12760 |
| 5736 | CGCUCACC A CCCAAAGC | 4012 | GCTTTGGG GGCTAGCTACAACGA GGTGAGCG | 12761 |
| 5743 | CACCCAAA G CACCCUCC | 4013 | GGAGGGTG GGCTAGCTACAACGA TTTGGGTG | 12762 |
| 5745 | CCCAAAGC A CCCUCCUG | 4014 | CAGGAGGG GGCTAGCTACAACGA GCTTTGGG | 12763 |
| 5753 | ACCCUCCU G UUCAACAU | 4015 | ATGTTGAA GGCTAGCTACAACGA AGGAGGGT | 12764 |
| 5758 | CCUGUUCA A CAUCUUGG | 4016 | CCAAGATG GGCTAGCTACAACGA TGAACAGG | 12765 |
| 5760 | UGUUCAAC A UCUUGGGA | 4017 | TCCCAAGA GGCTAGCTACAACGA GTTGAACA | 12766 |
| 5771 | UUGGGAGG G UGGGUGGC | 4018 | GCCACCCA GGCTAGCTACAACGA CCTCCCAA | 12767 |
| 5775 | GAGGGUGG G UGGCCGCC | 4019 | GGCGGCCA GGCTAGCTACAACGA CCACCCTC | 12768 |
| 5778 | GGUGGGUG G CCGCCCAA | 4020 | TTGGGCGG GGCTAGCTACAACGA CACCCACC | 12769 |
| 5781 | GGGUGGCC G CCCAACUC | 4021 | GAGTTGGG GGCTAGCTACAACGA GGCCACCC | 12770 |
| 5786 | GCCGCCCA A CUCGCUCC | 4022 | GGAGCGAG GGCTAGCTACAACGA TGGGCGGC | 12771 |
| 5790 | CCCAACUC G CUCCCCCC | 4023 | GGGGGGAG GGCTAGCTACAACGA GAGTTGGG | 12772 |
| 5802 | CCCCCAGA G CCGUUUCG | 4024 | CGAAACGG GGCTAGCTACAACGA TCTGGGGG | 12773 |
| 5805 | CCAGAGCC G UUUCGGCC | 4025 | GGCCGAAA GGCTAGCTACAACGA GGCTCTGG | 12774 |
| 5811 | CCGUUUCG G CCUUCGUG | 4026 | CACGAAGG GGCTAGCTACAACGA CGAAACGG | 12775 |
| 5817 | CGGCCUUC G UGGGCGCC | 4027 | GGCGCCCA GGCTAGCTACAACGA GAAGGCCG | 12776 |
| 5821 | CUUCGUGG G CGCCGGCA | 4028 | TGCCGGCG GGCTAGCTACAACGA CCACGAAG | 12777 |
| 5823 | UCGUGGGC G CCGGCAUC | 4029 | GATGCCGG GGCTAGCTACAACGA GCCCACGA | 12778 |
| 5827 | GGGCGCCG G CAUCGCUG | 4030 | CAGCGATG GGCTAGCTACAACGA CGGCGCCC | 12779 |
| 5829 | GCGCCGGC A UCGCUGGC | 4031 | GCCAGCGA GGCTAGCTACAACGA GCCGGCGC | 12780 |
| 5832 | CCGGCAUC G CUGGCGCG | 4032 | CGCGCCAG GGCTAGCTACAACGA GATGCCGG | 12781 |
| 5836 | CAUCGCUG G CGCGGCUG | 4033 | CAGCCGCG GGCTAGCTACAACGA CAGCGATG | 12782 |
| 5838 | UCGCUGGC G CGGCUGUU | 4034 | AACAGCCG GGCTAGCTACAACGA GCCAGCGA | 12783 |
| 5841 | CUGGCGCG G CUGUUGGC | 4035 | GCCAACAG GGCTAGCTACAACGA CGCGCCAG | 12784 |
| 5844 | GCGCGGCU G UUGGCAGC | 4036 | GCTGCCAA GGCTAGCTACAACGA AGCCGCGC | 12785 |
| 5848 | GGCUGUUG G CAGCAUAG | 4037 | CTATGCTG GGCTAGCTACAACGA CAACAGCC | 12786 |
| 5851 | UGUUGGCA G CAUAGGCC | 4038 | GGCCTATG GGCTAGCTACAACGA TGCCAACA | 12787 |
| 5853 | UUGGCAGC A UAGGCCUU | 4039 | AAGGCCTA GGCTAGCTACAACGA GCTGCCAA | 12788 |
| 5857 | CAGCAUAG G CCUUGGGA | 4040 | TCCCAAGG GGCTAGCTACAACGA CTATGCTG | 12789 |
| 5868 | UUGGGAAG G UGCUUGUA | 4041 | TACAAGCA GGCTAGCTACAACGA CTTCCCAA | 12790 |
| 5870 | GGGAAGGU G CUUGUAGA | 4042 | TCTACAAG GGCTAGCTACAACGA ACCTTCCC | 12791 |
| 5874 | AGGUGCUU G UAGACAUU | 4043 | AATGTCTA GGCTAGCTACAACGA AAGCACCT | 12792 |
| 5878 | GCUUGUAG A CAUUCUGG | 4044 | CCAGAATG GGCTAGCTACAACGA CTACAAGC | 12793 |
| 5880 | UUGUAGAC A UUCUGGCG | 4045 | CGCCAGAA GGCTAGCTACAACGA GTCTACAA | 12794 |
| 5886 | ACAUUCUG G CGGGCUAU | 4046 | ATAGCCCG GGCTAGCTACAACCA CAGAATGT | 12795 |
| 5890 | UCUGGCGC G CUAUGGAG | 4047 | CTCCATAG GGCTAGCTACAACGA CCGCCAGA | 12796 |
| 5893 | GGCGGGCU A UGGAGCAG | 4048 | CTGCTCCA GGCTAGCTACAACGA AGCCCGCC | 12797 |
| 5898 | GCUAUGCA G CAGGAGUG | 4049 | CACTCCTG GGCTAGCTACAACGA TCCATAGC | 12798 |
| 5904 | GAGCAGGA G UGGCGGGU | 4050 | ACCCGCCA GGCTAGCTACAACGA TCCTGCTC | 12799 |
| 5907 | CAGGAGUG G CGGGUGCU | 4051 | AGCACCCG GGCTAGCTACAACGA CACTCCTG | 12800 |
| 5911 | AGUGGCGG G UGCUCUCG | 4052 | CGAGAGCA GGCTAGCTACAACGA CCGCCACT | 12801 |
| 5913 | UGGCGGGU G CUCUCGUG | 4053 | CACGAGAG GGCTAGCTACAACGA ACCCGCCA | 12802 |
| 5919 | GUGCUCUC G UGGCCUUC | 4054 | GAAGGCCA GGCTAGCTACAACGA GAGAGCAC | 12803 |
| 5922 | CUCUCCUG G CCUUCAAG | 4055 | CTTGAAGG GGCTAGCTACAACGA CACGAGAG | 12804 |
| 5931 | CCUUCAAG G UCAUGAGC | 4056 | GCTCATGA GGCTAGCTACAACGA CTTGAAGG | 12805 |
| 5934 | UCAAGGUC A UGAGCGGG | 4057 | CCCGCTCA GGCTAGCTACAACGA GACCTTGA | 12806 |
| 5938 | GGUCAUGA G CGGGGAGA | 4058 | TCTCCCCG GGCTAGCTACAACGA TCATGACC | 12807 |
| 5946 | GCGGGGAG A UGCCUUCU | 4059 | AGAAGGCA GGCTAGCTACAACGA CTCCCCGC | 12808 |
| 5948 | GGGGAGAU G CCUUCUAC | 4060 | GTAGAAGG GGCTAGCTACAACGA ATCTCCCC | 12809 |
| 5955 | UGCCUUCU A CCGAGGAC | 4061 | GTCCTCGG GGCTAGCTACAACGA AGAAGGCA | 12810 |
| 5962 | UACCGAGG A CCUGGUCA | 4062 | TGACCAGG GGCTAGCTACAACGA CCTCGGTA | 12811 |
| 5967 | AGGACCUG G UCAACUUA | 4063 | TAAGTTGA GGCTAGCTACAACGA CAGGTCCT | 12812 |
| 5971 | CCUGGUCA A CUUACUCC | 4064 | GGAGTAAG GGCTAGCTACAACGA TGACCAGG | 12813 |
| 5975 | GUCAACUU A CUCCCUGC | 4065 | GCAGGGAG GGCTAGCTACAACGA AAGTTGAC | 12814 |
| 5982 | UACUCCCU G CCAUCCUC | 4066 | GAGGATGG GGCTAGCTACAACGA AGGGAGTA | 12815 |
| 5985 | UCCCUGCC A UCCUCUCU | 4067 | AGAGAGGA GGCTAGCTACAACGA GGCAGGGA | 12816 |
| 5998 | CUCUCCUG G CGCCCUGG | 4068 | CCAGGGCG GGCTAGCTACAACGA CAGGAGAG | 12817 |
| 6000 | CUCCUGGC G CCCUGGUC | 4069 | GACCAGGG GGCTAGCTACAACGA GCCAGGAG | 12818 |
| 6006 | GCGCCCUG G UCGUCGGG | 4070 | CCCGACGA GGCTAGCTACAACGA CAGGGCGC | 12819 |
| 6009 | CCCUGGUC G UCGGGGUG | 4071 | CACCCCGA GGCTAGCTACAACGA GACCAGGG | 12820 |
| 6015 | UCGUCGGG G UGGUGUGC | 4072 | GCACACCA GGCTAGCTACAACGA CCCGACGA | 12821 |
| 6018 | UCGGGGUG G UGUGCGCA | 4073 | TGCGCACA GGCTAGCTACAACGA CACCCCGA | 12822 |
| 6020 | GGGGUGGU G UGCGCAGC | 4074 | GCTGCGCA GGCTAGCTACAACGA ACCACCCC | 12823 |
| 6022 | GGUGGUGU G CGCAGCGA | 4075 | TCGCTGCG GGCTAGCTACAACGA ACACCACC | 12824 |
| 6024 | UGGUGUGC G CAGCGAUA | 4076 | TATCGCTG GGCTAGCTACAACGA GCACACCA | 12825 |
| 6027 | UGUGCGCA G CGAUACUG | 4077 | CAGTATCG GGCTAGCTACAACGA TGCGCACA | 12826 |
| 6030 | GCGCAGCG A UACUGCGU | 4078 | ACGCAGTA GGCTAGCTACAACGA CGCTGCGC | 12827 |
| 6032 | GCAGCGAU A CUGCGUCG | 4079 | CGACGCAG GGCTAGCTACAACGA ATCGCTGC | 12828 |
| 6035 | GCGAUACU G CGUCGGCA | 4080 | TGCCGACG GGCTAGCTACAACGA AGTATCGC | 12829 |
| 6037 | GAUACUGC G UCGGCAUG | 4081 | CATGCCGA GGCTAGCTACAACGA GCAGTATC | 12830 |
| 6041 | CUGCGUCG G CAUGUGGG | 4082 | CCCACATG GGCTAGCTACAACGA CGACGCAG | 12831 |
| 6043 | GCGUCGGC A UGUGGGCC | 4083 | GGCCCACA GGCTAGCTACAACGA GCCGACGC | 12832 |
| 6045 | GUCGGCAU G UGGGCCCA | 4084 | TGGGCCCA GGCTAGCTACAACGA ATGCCGAC | 12833 |
| 6049 | GCAUGUGG G CCCAGGAG | 4085 | CTCCTGGG GGCTAGCTACAACGA CCACATGC | 12834 |
| 6061 | AGGAGAGG G CGCUGUGC | 4086 | GCACAGCG GGCTAGCTACAACGA CCTCTCCT | 12835 |
| 6063 | GAGAGGGC G CUGUGCAG | 4087 | CTGCACAG GGCTAGCTACAACGA GCCCTCTC | 12836 |
| 6066 | AGGGCGCU G UGCAGUGG | 4088 | CCACTGCA GGCTAGCTACAACGA AGCGCCCT | 12837 |
| 6068 | GGCGCUGU G CAGUGGAU | 4089 | ATCCACTG GGCTAGCTACAACGA ACAGCGCC | 12838 |
| 6071 | GCUGUGCA G UGGAUGAA | 4090 | TTCATCCA GGCTAGCTACAACGA TGCACAGC | 12839 |
| 6075 | UGCAGUGG A UGAAUCGG | 4091 | CCGATTCA GGCTAGCTACAACGA CCACTGCA | 12840 |
| 6079 | GUGGAUGA A UCGGCUGA | 4092 | TCAGCCGA GGCTAGCTACAACGA TCATCCAC | 12841 |
| 6083 | AUGAAUCG G CUGAUAGC | 4093 | GCTATCAG GGCTAGCTACAACGA CGATTCAT | 12842 |
| 6087 | AUCGGCUG A UAGCGUUC | 4094 | GAACGCTA GGCTAGCTACAACGA CAGCCGAT | 12843 |
| 6090 | GGCUGAUA G CGUUCGCU | 4095 | AGCGAACG GGCTAGCTACAACGA TATCAGCC | 12844 |
| 6092 | CUGAUAGC G UUCGCUUC | 4096 | GAAGCGAA GGCTAGCTACAACGA GCTATCAG | 12845 |
| 6096 | UAGCGUUC G CUUCGCGG | 4097 | CCGCGAAG GGCTAGCTACAACGA GAACGCTA | 12846 |
| 6101 | UUCGCUUC G CGGGGCAA | 4098 | TTGCCCCG GGCTAGCTACAACGA GAAGCGAA | 12847 |
| 6106 | UUCGCGGG G CAACCAUG | 4099 | CATGGTTG GGCTAGCTACAACGA CCCGCGAA | 12848 |
| 6109 | GCGGGGCA A CCAUGUCU | 4100 | AGACATGG GGCTAGCTACAACGA TGCCCCGC | 12849 |
| 6112 | GGGCAACC A UGUCUCCC | 4101 | GGGAGACA GGCTAGCTACAACGA GGTTGCCC | 12850 |
| 6114 | GCAACCAU G UCUCCCCC | 4102 | GGGGGAGA GGCTAGCTACAACGA ATGGTTGC | 12851 |
| 6123 | UCUCCCCC A CGCACUAU | 4103 | ATAGTGCG GGCTAGCTACAACGA GGGGGAGA | 12852 |
| 6125 | UCCCCCAC G CACUAUGU | 4104 | ACATAGTG GGCTAGCTACAACGA GTGGGGGA | 12853 |
| 6127 | CCCCACGC A CUAUGUGC | 4105 | GCACATAG GGCTAGCTACAACGA GCGTGGGG | 12854 |
| 6130 | CACGCACU A UGUGCCUG | 4106 | CAGGCACA GGCTAGCTACAACGA AGTGCGTG | 12855 |
| 6132 | CGCACUAU G UGCCUGAG | 4107 | CTCAGGCA GGCTAGCTACAACGA ATAGTGCG | 12856 |
| 6134 | CACUAUGU G CCUGAGAG | 4108 | CTCTCAGG GGCTAGCTACAACGA ACATAGTG | 12857 |
| 6142 | GCCUGAGA G CGACGCAG | 4109 | CTGCGTCG GGCTAGCTACAACGA TCTCAGGC | 12858 |
| 6145 | UGAGAGCG A CGCAGCGG | 4110 | CCGCTGCG GGCTAGCTACAACGA CGCTCTCA | 12859 |
| 6147 | AGAGCGAC G CAGCGGCG | 4111 | CGCCGCTG GGCTAGCTACAACGA GTCGCTCT | 12860 |
| 6150 | GCGACGCA G CGGCGCGC | 4112 | GCGCGCCG GGCTAGCTACAACGA TGCGTCGC | 12861 |
| 6153 | ACGCAGCG G CGCGCGUC | 4113 | GACGCGCG GGCTAGCTACAACGA CGCTGCGT | 12862 |
| 6155 | GCAGCGGC G CGCGUCAC | 4114 | GTGACGCG GGCTAGCTACAACGA GCCGCTGC | 12863 |
| 6157 | AGCGGCGC G CGUCACAC | 4115 | GTGTGACG GGCTAGCTACAACGA GCGCCGCT | 12864 |
| 6159 | CGGCGCGC G UCACACAA | 4116 | TTGTGTGA GGCTAGCTACAACGA GCGCGCCG | 12865 |
| 6162 | CGCGCGUC A CACAAAUC | 4117 | GATTTGTG GGCTAGCTACAACGA GACGCGCG | 12866 |
| 6164 | CGCGUCAC A CAAAUCCU | 4118 | AGGATTTG GGCTAGCTACAACGA GTGACGCG | 12867 |
| 6168 | UCACACAA A UCCUCUCC | 4119 | GGAGAGGA GGCTAGCTACAACGA TTGTGTGA | 12868 |
| 6178 | CCUCUCCA G CCUCACCA | 4120 | TGGTGAGG GGCTAGCTACAACGA TGGAGAGG | 12869 |
| 6183 | CCAGCCUC A CCAUCACU | 4121 | AGTGATGG GGCTAGCTACAACGA GAGGCTGG | 12870 |
| 6186 | GCCUCACC A UCACUCAG | 4122 | CTGAGTGA GGCTAGCTACAACGA GGTGAGGC | 12871 |
| 6189 | UCACCAUC A CUCAGCUG | 4123 | CAGCTGAG GGCTAGCTACAACGA GATGGTGA | 12872 |
| 6194 | AUCACUCA G CUGCUGAG | 4124 | CTCAGCAG GGCTAGCTACAACGA TGAGTGAT | 12873 |
| 6197 | ACUCAGCU G CUGAGGAG | 4125 | CTCCTCAG GGCTAGCTACAACGA AGCTGAGT | 12874 |
| 6206 | CUGAGGAG G CUCCAUCA | 4126 | TGATGGAG GGCTAGCTACAACGA CTCCTCAG | 12875 |
| 6211 | GAGGCUCC A UCAGUGGA | 4127 | TCCACTGA GGCTAGCTACAACGA GGAGCCTC | 12876 |
| 6215 | CUCCAUCA G UGGAUCAA | 4128 | TTGATCCA GGCTAGCTACAACGA TGATGGAG | 12877 |
| 6219 | AUCAGUGG A UCAAUGAG | 4129 | CTCATTGA GGCTAGCTACAACGA CCACTGAT | 12878 |
| 6223 | GUGGAUCA A UGAGGACU | 4130 | AGTCCTCA GGCTAGCTACAACGA TGATCCAC | 12879 |
| 6229 | CAAUGAGG A CUGCUCCA | 4131 | TGGAGCAG GGCTAGCTACAACGA CCTCATTG | 12880 |
| 6232 | UGAGGACU G CUCCACGC | 4132 | GCGTGGAG GGCTAGCTACAACGA AGTCCTCA | 12881 |
| 6237 | ACUGCUCC A CGCCAUGU | 4133 | ACATGGCG GGCTAGCTACAACGA GGAGCAGT | 12882 |
| 6239 | UGCUCCAC G CCAUGUUC | 4134 | GAACATGG GGCTAGCTACAACGA GTGGAGCA | 12883 |
| 6242 | UCCACGCC A UGUUCCGG | 4135 | CCGGAACA GGCTAGCTACAACGA GGCGTGGA | 12884 |
| 6244 | CACGCCAU G UUCCGGCU | 4136 | AGCCGGAA GGCTAGCTACAACGA ATGGCGTG | 12885 |
| 6250 | AUGUUCCG G CUCGUGGC | 4137 | GCCACGAG GGCTAGCTACAACGA CGGAACAT | 12886 |
| 6254 | UCCGGCUC G UGGCUAAG | 4138 | CTTAGCCA GGCTAGCTACAACGA GAGCCGGA | 12887 |
| 6257 | GGCUCGUG G CUAAGGGA | 4139 | TCCCTTAG GGCTAGCTACAACGA CACGAGCC | 12888 |
| 6265 | GCUAAGGG A UGUUUGGG | 4140 | CCCAAACA GGCTAGCTACAACGA CCCTTAGC | 12889 |
| 6267 | UAAGGGAU G UUUGGGAC | 4141 | GTCCCAAA GGCTAGCTACAACGA ATCCCTTA | 12890 |
| 6274 | UGUUUGGG A CUGGAUAU | 4142 | ATATCCAG GGCTAGCTACAACGA CCCAAACA | 12891 |
| 6279 | GGGACUGG A UAUGCACG | 4143 | CGTGCATA GGCTAGCTACAACGA CCAGTCCC | 12892 |
| 6281 | GACUGGAU A UGCACGGU | 4144 | ACCOTGCA GGCTAGCTACAACGA ATCCAGTC | 12893 |
| 6283 | CUGGAUAU G CACGGUGU | 4145 | ACACCGTG GGCTAGCTACAACGA ATATCCAG | 12894 |
| 6285 | GGAUAUGC A CGGUGUUG | 4146 | CAACACCG GGCTAGCTACAACGA GCATATCC | 12895 |
| 6288 | UAUGCACG G UGUUGACU | 4147 | AGTCAACA GGCTAGCTACAACGA CGTGCATA | 12896 |
| 6290 | UGCACGGU G UUGACUGA | 4148 | TCAGTCAA GGCTAGCTACAACGA ACCGTGCA | 12897 |
| 6294 | CGGUGUUG A CUGACUUC | 4149 | GAAGTCAG GGCTAGCTACAACGA CAACACCG | 12898 |
| 6298 | GUUGACUG A CUUCAAGA | 4150 | TCTTGAAG GGCTAGCTACAACGA CAGTCAAC | 12899 |
| 6306 | ACUUCAAG A CCUGGCUU | 4151 | AAGCCAGG GGCTAGCTACAACGA CTTGAAGT | 12900 |
| 6311 | AAGACCUG G CUUCAGUC | 4152 | GACTGAAG GGCTAGCTACAACGA CAGGTCTT | 12901 |
| 6317 | UGGCUUCA G UCCAAGCU | 4153 | AGCTTGGA GGCTAGCTACAACGA TGAAGCCA | 12902 |
| 6323 | CAGUCCAA G CUCCUGCC | 4154 | GGCAGGAG GGCTAGCTACAACGA TTGGACTG | 12903 |
| 6329 | AAGCUCCU G CCGCGGUU | 4155 | AACCGCGG GGCTAGCTACAACGA AGGAGCTT | 12904 |
| 6332 | CUCCUGCC G CGGUUGCC | 4156 | GGCAACCG GGCTAGCTACAACGA GGCAGGAG | 12905 |
| 6335 | CUGCCGCG G UUGCCGGG | 4157 | CCCGGCAA GGCTAGCTACAACGA CGCGGCAG | 12906 |
| 6338 | CCGCGGUU G CCGGGAGU | 4158 | ACTCCCGG GGCTAGCTACAACGA AACCGCGG | 12907 |
| 6345 | UGCCCGGA G UCCCUUUC | 4159 | GAAAGGGA GGCTAGCTACAACGA TCCCGGCA | 12908 |
| 6359 | UUCUUCUC A UGCCAACG | 4160 | CGTTGGCA GGCTAGCTACAACGA GAGAAGAA | 12909 |
| 6361 | CUUCUCAU G CCAACGUG | 4161 | CACGTTGG GGCTAGCTACAACGA ATGAGAAG | 12910 |
| 6365 | UCAUGCCA A CGUGGGUA | 4162 | TACCCACG GGCTAGCTACAACGA TGGCATGA | 12911 |
| 6367 | AUGCCAAC G UGGGUACA | 4163 | TGTACCCA GGCTAGCTACAACGA GTTGGCAT | 12912 |
| 6371 | CAACGUGG G UACAGGGG | 4164 | CCCCTGTA GGCTAGCTACAACGA CCACGTTG | 12913 |
| 6373 | ACGUGGGU A CAGGGGGG | 4165 | CCCCCCTG GGCTAGCTACAACGA ACCCACGT | 12914 |
| 6381 | ACAGGGGG G UCUGGCGG | 4166 | CCGCCAGA GGCTAGCTACAACGA CCCCCTGT | 12915 |
| 6386 | GGGGUCUG G CGGGGAGA | 4167 | TCTCCCCG GGCTAGCTACAACGA CAGACCCC | 12916 |
| 6394 | GCGGGGAG A CGGUAUCA | 4168 | TGATACCG GGCTAGCTACAACGA CTCCCCGC | 12917 |
| 6397 | GGGAGACG G UAUCAUGC | 4169 | GCATGATA GGCTAGCTACAACGA CGTCTCCC | 12918 |
| 6399 | GAGACGGU A UCAUGCAA | 4170 | TTGCATGA GGCTAGCTACAACGA ACCGTCTC | 12919 |
| 6402 | ACGGUAUC A UGCAAACC | 4171 | GGTTTGCA GGCTAGCTACAACGA GATACCGT | 12920 |
| 6404 | GGUAUCAU G CAAACCAC | 4172 | GTGGTTTG GGCTAGCTACAACGA ATGATACC | 12921 |
| 6408 | UCAUGCAA A CCACCUGC | 4173 | GCAGGTGG GGCTAGCTACAACGA TTGCATGA | 12922 |
| 6411 | UGCAAACC A CCUGCCCA | 4174 | TGGGCAGG GGCTAGCTACAACGA GGTTTGCA | 12923 |
| 6415 | AACCACCU G CCCAUGCG | 4175 | CGCATGGG GGCTAGCTACAACGA AGGTGGTT | 12924 |
| 6419 | ACCUGCCC A UGCGGAGC | 4176 | GCTCCGCA GGCTAGCTACAACGA GGGCAGGT | 12925 |
| 6421 | CUGCCCAU G CGGAGCGC | 4177 | GCGCTCCG GGCTAGCTACAACGA ATGGGCAG | 12926 |
| 6426 | CAUGCGGA G CGCAGAUC | 4178 | GATCTGCG GGCTAGCTACAACGA TCCGCATG | 12927 |
| 6428 | UGCGGAGC G CAGAUCAC | 4179 | GTGATCTG GGCTAGCTACAACGA GCTCCGCA | 12928 |
| 6432 | GAGCGCAG A UCACUGGA | 4180 | TCCAGTGA GGCTAGCTACAACGA CTGCGCTC | 12929 |
| 6435 | CGCAGAUC A CUGGACAU | 4181 | ATGTCCAG GGCTAGCTACAACGA GATCTGCG | 12930 |
| 6440 | AUCACUGG A CAUGUCAA | 4182 | TTGACATG GGCTAGCTACAACGA CCAGTGAT | 12931 |
| 6442 | CACUGGAC A UGUCAAGA | 4183 | TCTTGACA GGCTAGCTACAACGA GTCCAGTG | 12932 |
| 6444 | CUGGACAU G UCAAGAAC | 4184 | GTTCTTGA GGCTAGCTACAACGA ATGTCCAG | 12933 |
| 6451 | UGUCAAGA A CGGUUCCA | 4185 | TGGAACCG GGCTAGCTACAACGA TCTTGACA | 12934 |
| 6454 | CAAGAACG G UUCCAUGA | 4186 | TCATGGAA GGCTAGCTACAACGA CGTTCTTG | 12935 |
| 6459 | ACGGUUCC A UGAGGAUC | 4187 | GATCCTCA GGCTAGCTACAACGA GGAACCGT | 12936 |
| 6465 | CCAUGAGG A UCGUCGGG | 4188 | CCCGACGA GGCTAGCTACAACGA CCTCATGG | 12937 |
| 6468 | UGAGGAUC G UCGGGCCU | 4189 | AGGCCCGA GGCTAGCTACAACGA GATCCTCA | 12938 |
| 6473 | AUCGUCGG G CCUAAGAC | 4190 | GTCTTAGG GGCTAGCTACAACGA CCGACGAT | 12939 |
| 6480 | GGCCUAAG A CCUGUAGC | 4191 | GCTACAGG GGCTAGCTACAACGA CTTAGGCC | 12940 |
| 6484 | UAAGACCU G UAGCAACA | 4192 | TGTTGCTA GGCTAGCTACAACGA AGGTCTTA | 12941 |
| 6487 | GACCUGUA G CAACACGU | 4193 | ACGTGTTG GGCTAGCTACAACGA TACAGGTC | 12942 |
| 6490 | CUGUAGCA A CACGUGGC | 4194 | GCCACGTG GGCTAGCTACAACGA TGCTACAG | 12943 |
| 6492 | GUAGCAAC A CGUGGCAU | 4195 | ATGCCACG GGCTAGCTACAACGA GTTGCTAC | 12944 |
| 6494 | AGCAACAC G UGGCAUGG | 4196 | CCATGCCA GGCTAGCTACAACGA GTGTTGCT | 12945 |
| 6497 | AACACGUG G CAUGGAAC | 4197 | GTTCCATG GGCTAGCTACAACGA CACGTGTT | 12946 |
| 6499 | CACGUGGC A UGGAACAU | 4198 | ATGTTCCA GGCTAGCTACAACGA GCCACGTG | 12947 |
| 6504 | GGCAUGGA A CAUUCCCC | 4199 | GGGGAATG GGCTAGCTACAACGA TCCATGCC | 12948 |
| 6506 | CAUGGAAC A UUCCCCAU | 4200 | ATGGGGAA GGCTAGCTACAACGA GTTCCATG | 12949 |
| 6513 | CAUUCCCC A UCAACGCA | 4201 | TGCGTTGA GGCTAGCTACAACGA GGGGAATG | 12950 |
| 6517 | CCCCAUCA A CGCAUACA | 4202 | TGTATGCG GGCTAGCTACAACGA TGATGGGG | 12951 |
| 6519 | CCAUCAAC G CAUACACC | 4203 | GGTGTATG GGCTAGCTACAACGA GTTGATGG | 12952 |
| 6521 | AUCAACGC A UACACCAC | 4204 | GTGGTGTA GGCTAGCTACAACGA GCGTTGAT | 12953 |
| 6523 | CAACGCAU A CACCACGG | 4205 | CCGTGGTG GGCTAGCTACAACGA ATGCGTTG | 12954 |
| 6525 | ACGCAUAC A CCACGGGC | 4206 | GCCCGTGG GGCTAGCTACAACGA GTATGCGT | 12955 |
| 6528 | CAUACACC A CGGGCCCC | 4207 | GGGGCCCG GGCTAGCTACAACGA GGTGTATG | 12956 |
| 6532 | CACCACGG G CCCCUGCA | 4208 | TGCAGGGG GGCTAGCTACAACGA CCGTGGTG | 12957 |
| 6538 | GGCCCCCU G CACACCCU | 4209 | AGGGTGTG GGCTAGCTACAACGA AGGGGCCC | 12958 |
| 6540 | GCCCCUGC A CACCCUCC | 4210 | GGAGGGTG GGCTAGCTACAACGA GCAGGGGC | 12959 |
| 6542 | CCCUGCAC A CCCUCCCC | 4211 | GGGGAGGG GGCTAGCTACAACGA GTGCAGGG | 12960 |
| 6552 | CCUCCCCG G CGCCAAAC | 4212 | GTTTGGCG GGCTAGCTACAACGA CGGGGAGG | 12961 |
| 6554 | UCCCCGGC G CCAAACUA | 4213 | TAGTTTGG GGCTAGCTACAACGA GCCGGGGA | 12962 |
| 6559 | GGCGCCAA A CUAUUCUA | 4214 | TAGAATAG GGCTAGCTACAACGA TTGGCGCC | 12963 |
| 6562 | GCCAAACU A UUCUAGGG | 4215 | CCCTAGAA GGCTAGCTACAACGA AGTTTGGC | 12964 |
| 6570 | AUUCUAGG G CGCUAUGG | 4216 | CCATAGCG GGCTAGCTACAACGA CCTAGAAT | 12965 |
| 6572 | UCUAGGGC G CUAUGGCG | 4217 | CGCCATAG GGCTAGCTACAACGA GCCCTAGA | 12966 |
| 6575 | AGGGCGCU A UGGCGGGU | 4218 | ACCCGCCA GGCTAGCTACAACGA AGCGCCCT | 12967 |
| 6578 | GCGCUAUG G CGGGUGGC | 4219 | GCCACCCG GGCTAGCTACAACGA CATAGCGC | 12968 |
| 6582 | UAUGGCGG G UGGCCGCU | 4220 | AGCGGCCA GGCTAGCTACAACGA CCGCCATA | 12969 |
| 6585 | GGCGGGUG G CCGCUGAG | 4221 | CTCAGCGG GGCTAGCTACAACGA CACCCGCC | 12970 |
| 6588 | GGGUGGCC G CUGAGGAG | 4222 | CTCCTCAG GGCTAGCTACAACGA GGCCACCC | 12971 |
| 6596 | GCUGAGGA G UACGUGGA | 4223 | TCCACGTA GGCTAGCTACAACGA TCCTCAGC | 12972 |
| 6598 | UGAGGAGU A CGUGGAGG | 4224 | CCTCCACG GGCTAGCTACAACGA ACTCCTCA | 12973 |
| 6600 | AGGAGUAC G UGGAGGUU | 4225 | AACCTCCA GGCTAGCTACAACGA GTACTCCT | 12974 |
| 6606 | ACGUGGAG G UUACGCGG | 4226 | CCGCGTAA GGCTAGCTACAACGA CTCCACGT | 12975 |
| 6609 | UGGAGGUU A CGCGGGUG | 4227 | CACCCGCG GGCTAGCTACAACGA AACCTCCA | 12976 |
| 6611 | GAGGUUAC G CGGGUGGG | 4228 | CCCACCCG GGCTAGCTACAACGA GTAACCTC | 12977 |
| 6615 | UUACGCGG G UGGGGGAU | 4229 | ATCCCCCA GGCTAGCTACAACGA CCGCGTAA | 12978 |
| 6622 | GGUGGGGG A UUUCCACU | 4230 | AGTGGAAA GGCTAGCTACAACGA CCCCCACC | 12979 |
| 6628 | GGAUUUCC A CUACGUGA | 4231 | TCACGTAG GGCTAGCTACAACGA GGAAATCC | 12980 |
| 6631 | UUUCCACU A CGUGACGG | 4232 | CCGTCACG GGCTAGCTACAACGA AGTGGAAA | 12981 |
| 6633 | UCCACUAC G UGACGGGC | 4233 | GCCCGTCA GGCTAGCTACAACGA GTAGTGGA | 12982 |
| 6636 | ACUACGUG A CGGGCAUG | 4234 | CATGCCCG GGCTAGCTACAACGA CACGTAGT | 12983 |
| 6640 | CGUGACGG G CAUGACCA | 4235 | TGGTCATG GGCTAGCTACAACGA CCGTCACG | 12984 |
| 6642 | UGACGGGC A UGACCACU | 4236 | AGTGGTCA GGCTAGCTACAACGA GCCCGTCA | 12985 |
| 6645 | CGGGCAUG A CCACUGAC | 4237 | GTCAGTGG GGCTAGCTACAACGA CATGCCCG | 12986 |
| 6648 | GCAUGACC A CUGACAAC | 4238 | GTTGTCAG GGCTAGCTACAACGA GGTCATGC | 12987 |
| 6652 | GACCACUG A CAACGUAA | 4239 | TTACGTTG GGCTAGCTACAACGA CAGTGGTC | 12988 |
| 6655 | CACUGACA A CGUAAAAU | 4240 | ATTTTACG GGCTAGCTACAACGA TGTCAGTG | 12989 |
| 6657 | CUGACAAC G UAAAAUGC | 4241 | GCATTTTA GGCTAGCTACAACGA GTTGTCAG | 12990 |
| 6662 | AACGUAAA A UGCCCGUG | 4242 | CACGGGCA GGCTAGCTACAACGA TTTACGTT | 12991 |
| 6664 | CGUAAAAU G CCCGUGCC | 4243 | GGCACGGG GGCTAGCTACAACGA ATTTTACG | 12992 |
| 6668 | AAAUGCCC G UGCCAGGU | 4244 | ACCTGGCA GGCTAGCTACAACGA GGGCATTT | 12993 |
| 6670 | AUGCCCGU G CCAGGUUC | 4245 | GAACCTGG GGCTAGCTACAACGA ACGGGCAT | 12994 |
| 6675 | CGUGCCAG G UUCCGCCC | 4246 | GGGCGGAA GGCTAGCTACAACGA CTGGCACG | 12995 |
| 6680 | CAGGUUCC G CCCCCCGA | 4247 | TCGGGGGG GGCTAGCTACAACGA GGAACCTG | 12996 |
| 6689 | CCCCCCGA A UUCUUCAC | 4248 | GTGAAGAA GGCTAGCTACAACGA TCGGGGGG | 12997 |
| 6696 | AAUUCUUC A CGGAAGUG | 4249 | CACTTCCG GGCTAGCTACAACGA GAAGAATT | 12998 |
| 6702 | UCACGGAA G UGGAUGGG | 4250 | CCCATCCA GGCTAGCTACAACGA TTCCGTGA | 12999 |
| 6706 | GGAAGUGG A UGGGGUAC | 4251 | GTACCCCA GGCTAGCTACAACGA CCACTTCC | 13000 |
| 6711 | UGGAUGGG G UACGCCUG | 4252 | CAGGCGTA GGCTAGCTACAACGA CCCATCCA | 13001 |
| 6713 | GAUGGGGU A CGCCUGCA | 4253 | TGCAGGCG GGCTAGCTACAACGA ACCCCATC | 13002 |
| 6715 | UGGGGUAC G CCUGCACA | 4254 | TGTGCAGG GGCTAGCTACAACGA GTACCCCA | 13003 |
| 6719 | GUACGCCU G CACAGAAA | 4255 | TTTCTGTG GGCTAGCTACAACGA AGGCGTAC | 13004 |
| 6721 | ACGCCUGC A CAGAAACG | 4256 | CGTTTCTG GGCTAGCTACAACGA GCAGGCGT | 13005 |
| 6727 | GCACAGAA A CGCUCCGG | 4257 | CCGGAGCG GGCTAGCTACAACGA TTCTGTGC | 13006 |
| 6729 | ACAGAAAC G CUCCGGCG | 4258 | CGCCGGAG GGCTAGCTACAACGA GTTTCTGT | 13007 |
| 6735 | ACGCUCCG G CGUGUGGA | 4259 | TCCACACG GGCTAGCTACAACGA CGGAGCGT | 13008 |
| 6737 | GCUCCGGC G UGUGGACC | 4260 | GGTCCACA GGCTAGCTACAACGA GCCGGAGC | 13009 |
| 6739 | UCCGGCGU G UGGACCUC | 4261 | GAGGTCCA GGCTAGCTACAACGA ACGCCGGA | 13010 |
| 6743 | GCGUGUGG A CCUCUCCU | 4262 | AGGAGAGG GGCTAGCTACAACGA CCACACGC | 13011 |
| 6752 | CCUCUCCU A CGGGAGGA | 4263 | TCCTCCCG GGCTAGCTACAACGA AGGAGAGG | 13012 |
| 6762 | GGGAGGAG G UCACAUUC | 4264 | GAATGTGA GGCTAGCTACAACGA CTCCTCCC | 13013 |
| 6765 | AGGAGGUC A CAUUCCAG | 4265 | CTGGAATG GGCTAGCTACAACGA GACCTCCT | 13014 |
| 6767 | GAGGUCAC A UUCCAGGU | 4266 | ACCTGGAA GGCTAGCTACAACGA GTGACCTC | 13015 |
| 6774 | CAUUCCAG G UCGGGCUC | 4267 | GAGCCCGA GGCTAGCTACAACGA CTGGAATG | 13016 |
| 6779 | CAGGUCGG G CUCAACCA | 4268 | TGGTTGAG GGCTAGCTACAACGA CCGACCTG | 13017 |
| 6784 | CGGGCUCA A CCAAUACC | 4269 | GGTATTGG GGCTAGCTACAACGA TGAGCCCG | 13018 |
| 6788 | CUCAACCA A UACCUGGU | 4270 | ACCAGGTA GGCTAGCTACAACGA TGGTTGAG | 13019 |
| 6790 | CAACCAAU A CCUGGUUG | 4271 | CAACCAGG GGCTAGCTACAACGA ATTGGTTG | 13020 |
| 6795 | AAUACCUG G UUGGGUCA | 4272 | TGACCCAA GGCTAGCTACAACGA CAGGTATT | 13021 |
| 6800 | CUGGUUGG G UCACAGCU | 4273 | AGCTGTGA GGCTAGCTACAACGA CCAACCAG | 13022 |
| 6803 | GUUGGGUC A CAGCUCCC | 4274 | GGGAGCTG GGCTAGCTACAACGA GACCCAAC | 13023 |
| 6806 | GGGUCACA G CUCCCAUG | 4275 | CATGGGAG GGCTAGCTACAACGA TGTGACCC | 13024 |
| 6812 | CAGCUCCC A UGCGAGCC | 4276 | GGCTCGCA GGCTAGCTACAACGA GGGAGCTG | 13025 |
| 6814 | GCUCCCAU G CGAGCCCG | 4277 | CGGGCTCG GGCTAGCTACAACGA ATGGGAGC | 13026 |
| 6818 | CCAUGCGA G CCCGAACC | 4278 | GGTTCGGG GGCTAGCTACAACGA TCGCATGG | 13027 |
| 6824 | GAGCCCGA A CCGGAUGU | 4279 | ACATCCGG GGCTAGCTACAACGA TCGGGCTC | 13028 |
| 6829 | CGAACCGG A UGUACCAG | 4280 | CTGCTACA GGCTAGCTACAACGA CCGGTTCG | 13029 |
| 6831 | AACCGGAU G UAGCAGUG | 4281 | CACTGCTA GGCTAGCTACAACGA ATCCGGTT | 13030 |
| 6834 | CGGAUGUA G CAGUGCUC | 4282 | GAGCACTC GGCTAGCTACAACGA TACATCCG | 13031 |
| 6837 | AUGUAGCA G UGCUCACG | 4283 | CGTGAGCA GGCTAGCTACAACGA TGCTACAT | 13032 |
| 6839 | GUAGCAGU G CUCACGUC | 4284 | GACGTGAG GGCTAGCTACAACGA ACTGCTAC | 13033 |
| 6843 | CAGUCCUC A CGUCCAUG | 4285 | CATGGACG GGCTAGCTACAACGA GAGCACTG | 13034 |
| 6845 | GUGCUCAC G UCCAUGCU | 4286 | AGCATGGA GGCTAGCTACAACGA GTGAGCAC | 13035 |
| 6849 | UCACGUCC A UGCUCACC | 4287 | GGTGAGCA GGCTAGCTACAACGA GGACGTGA | 13036 |
| 6851 | ACGUCCAU G CUCACCGA | 4288 | TCGGTGAG GGCTAGCTACAACGA ATGGACGT | 13037 |
| 6855 | CCAUGCUC A CCGACCCC | 4289 | GGGGTCGG GGCTAGCTACAACGA GAGCATGG | 13038 |
| 6859 | GCUCACCG A CCCCUCCC | 4290 | GGGAGGGG GGCTAGCTACAACGA CGGTGAGC | 13039 |
| 6868 | CCCCUCCC A CAUUACAG | 4291 | CTGTAATG GGCTAGCTACAACGA GGGAGGGG | 13040 |
| 6870 | CCUCCCAC A UUACAGGA | 4292 | TCCTGTAA GGCTAGCTACAACGA GTGGGAGG | 13041 |
| 6873 | CCCACAUU A CAGGAGAG | 4293 | CTCTCCTG GGCTAGCTACAACGA AATGTGGG | 13042 |
| 6882 | CAGGAGAG A CGGCUAAG | 4294 | CTTAGCCG GGCTAGCTACAACGA CTCTCCTG | 13043 |
| 6885 | GAGAGACG G CUAAGCGU | 4295 | ACGCTTAG GGCTAGCTACAACGA CGTCTCTC | 13044 |
| 6890 | ACGGCUAA G CGUAGGCU | 4296 | AGCCTACG GGCTAGCTACAACGA TTAGCCGT | 13045 |
| 6892 | GGCUAAGC G UAGGCUGG | 4297 | CCAGCCTA GGCTAGCTACAACGA GCTTAGCC | 13046 |
| 6896 | AAGCGUAG G CUGGCCAG | 4298 | CTGGCCAG GGCTAGCTACAACGA CTACGCTT | 13047 |
| 6900 | GUAGGCUG G CCAGGGGG | 4299 | CCCCCTGG GGCTAGCTACAACGA CAGCCTAC | 13048 |
| 6908 | GCCAGGGG G UCUCCCCC | 4300 | GGGGGAGA GGCTAGCTACAACGA CCCCTGGC | 13049 |
| 6924 | CCUCCUUG G CCAGCUCC | 4301 | GGAGCTGG GGCTAGCTACAACGA CAAGGAGG | 13050 |
| 6928 | CUUGGCCA G CUCCUCAG | 4302 | CTGAGGAG GGCTAGCTACAACGA TGGCCAAG | 13051 |
| 6936 | GCUCCUCA G CUAGCCAG | 4303 | CTGGCTAG GGCTAGCTACAACGA TGAGGAGC | 13052 |
| 6940 | CUCAGCUA G CCAGCUGU | 4304 | ACAGCTGG GGCTAGCTACAACGA TAGCTGAG | 13053 |
| 6944 | GCUAGCCA G CUGUCUGC | 4305 | GCAGACAG GGCTAGCTACAACGA TGGCTAGC | 13054 |
| 6947 | AGCCAGCU G UCUGCGCC | 4306 | GGCGCAGA GGCTAGCTACAACGA AGCTGGCT | 13055 |
| 6951 | AGCUGUCU G CGCCUUCU | 4307 | AGAAGGCG GGCTAGCTACAACGA AGACAGCT | 13056 |
| 6953 | CUGUCUGC G CCUUCUUC | 4308 | GAAGAAGG GGCTAGCTACAACGA GCAGACAG | 13057 |
| 6966 | CUUCGAAG G CGACAUAC | 4309 | GTATGTCG GGCTAGCTACAACGA CTTCGAAG | 13058 |
| 6969 | CGAAGGCG A CAUACAUU | 4310 | AATGTATG GGCTAGCTACAACGA CGCCTTCG | 13059 |
| 6971 | AAGGCGAC A UACAUUAC | 4311 | GTAATGTA GGCTAGCTACAACGA GTCGCCTT | 13060 |
| 6973 | GGCGACAU A CAUUACCC | 4312 | GGGTAATG GGCTAGCTACAACGA ATGTCGCC | 13061 |
| 6975 | CGACAUAC A UUACCCAA | 4313 | TTGGGTAA GGCTAGCTACAACGA GTATGTCG | 13062 |
| 6978 | CAUACAUU A CCCAAUAU | 4314 | ATATTGGG GGCTAGCTACAACGA AATGTATG | 13063 |
| 6983 | AUUACCCA A UAUGACUC | 4315 | GAGTCATA GGCTAGCTACAACGA TGGGTAAT | 13064 |
| 6985 | UACCCAAU A UGACUCCC | 4316 | GGGAGTCA GGCTAGCTACAACGA ATTGGGTA | 13065 |
| 6988 | CCAAUAUG A CUCCCCAG | 4317 | CTGGGGAG GGCTAGCTACAACGA CATATTGG | 13066 |
| 6997 | CUCCCCAG A CUUUGACC | 4318 | GGTCAAAG GGCTAGCTACAACGA CTGGGGAG | 13067 |
| 7003 | AGACUUUG A CCUCAUCG | 4319 | CGATGAGG GGCTAGCTACAACGA CAAAGTCT | 13068 |
| 7008 | UUGACCUC A UCGAGGCC | 4320 | GGCCTCGA GGCTAGCTACAACGA GAGGTCAA | 13069 |
| 7014 | UCAUCGAG G CCAACCUC | 4321 | GAGGTTGG GGCTAGCTACAACGA CTCGATGA | 13070 |
| 7018 | CGAGGCCA A CCUCCUGU | 4322 | ACAGGAGG GGCTAGCTACAACGA TGGCCTCG | 13071 |
| 7025 | AACCUCCU G UGGCGGCA | 4323 | TGCCGCCA GGCTAGCTACAACGA AGGAGGTT | 13072 |
| 7028 | CUCCUGUG G CGGCAGGA | 4324 | TCCTGCCG GGCTAGCTACAACGA CACAGGAG | 13073 |
| 7031 | CUGUGGCG G CAGGAGAU | 4325 | ATCTCCTG GGCTAGCTACAACGA CGCCACAG | 13074 |
| 7038 | GGCAGGAG A UGGGCGGU | 4326 | ACCGCCCA GGCTAGCTACAACGA CTCCTGCC | 13075 |
| 7042 | GGAGAUGG G CGGUAACA | 4327 | TGTTACCG GGCTAGCTACAACGA CCATCTCC | 13076 |
| 7045 | GAUGGGCG G UAACAUCA | 4328 | TGATGTTA GGCTAGCTACAACGA CGCCCATC | 13077 |
| 7048 | GGGCGCUA A CAUCACUC | 4329 | GAGTCATG GGCTAGCTACAACGA TACCGCCC | 13078 |
| 7050 | GCCGUAAC A UCACUCGC | 4330 | GCGAGTGA GGCTAGCTACAACGA GTTACCGC | 13079 |
| 7053 | GUAACAUC A CUCGCGUG | 4331 | CACGCGAG GGCTAGCTACAACGA GATGTTAC | 13080 |
| 7057 | CAUCACUC G CCUGGAGU | 4332 | ACTCCACG GGCTAGCTACAACGA GAGTGATG | 13081 |
| 7059 | UCACUCGC G UCCAGUCA | 4333 | TGACTCCA GGCTAGCTACAACGA GCGAGTGA | 13082 |
| 7064 | CGCGUGGA G UCAGAGAA | 4334 | TTCTCTGA GGCTAGCTACAACGA TCCACGCG | 13083 |
| 7072 | GUCAGAGA A UAAGGUAG | 4335 | CTACCTTA GGCTAGCTACAACGA TCTCTGAC | 13084 |
| 7077 | AGAAUAAG G UAGUUACC | 4336 | GGTAACTA GGCTAGCTACAACGA CTTATTCT | 13085 |
| 7080 | AUAAGGUA G UUACCCUG | 4337 | CAGGGTAA GGCTAGCTACAACGA TACCTTAT | 13086 |
| 7083 | AGGUAGUU A CCCUGGAC | 4338 | GTCCAGGG GGCTAGCTACAACGA AACTACCT | 13087 |
| 7090 | UACCCUGG A CUCUUUUG | 4339 | CAAAAGAG GGCTAGCTACAACGA CCAGGGTA | 13088 |
| 7099 | CUCUUUUG A CCCGCUUC | 4340 | GAAGCGGG GGCTAGCTACAACGA CAAAAGAG | 13089 |
| 7103 | UUUGACCC G CUUCGAGC | 4341 | GCTCGAAG GGCTAGCTACAACGA GGGTCAAA | 13090 |
| 7110 | CGCUUCGA G CGGAGGAG | 4342 | CTCCTCCG GGCTAGCTACAACGA TCGAAGCG | 13091 |
| 7120 | GGAGGAGG A UGAGAGAG | 4343 | CTCTCTCA GGCTAGCTACAACGA CCTCCTCC | 13092 |
| 7131 | AGAGAGAG G UGUCCAUU | 4344 | AATGGACA GGCTAGCTACAACGA CTCTCTCT | 13093 |
| 7133 | AGAGAGGU G UCCAUUCC | 4345 | GGAATGGA GGCTAGCTACAACGA ACCTCTCT | 13094 |
| 7137 | AGGUGUCC A UUCCGGCG | 4346 | CGCCGGAA GGCTAGCTACAACGA GGACACCT | 13095 |
| 7143 | CCAUUCCG G CGGAGAUC | 4347 | GATCTCCG GGCTAGCTACAACGA CGGAATGG | 13096 |
| 7149 | CGGCGGAG A UCCUGCGG | 4348 | CCGCAGGA GGCTAGCTACAACGA CTCCGCCG | 13097 |
| 7154 | GAGAUCCU G CGGAAAUC | 4349 | GATTTCCG GGCTAGCTACAACGA AGGATCTC | 13098 |
| 7160 | CUGCGGAA A UCCAAGAA | 4350 | TTCTTGGA GGCTAGCTACAACGA TTCCGCAG | 13099 |
| 7169 | UCCAAGAA G UUUCCUUC | 4351 | GAAGGAAA GGCTAGCTACAACGA TTCTTGGA | 13100 |
| 7179 | UUCCUUCA G CGUUACCC | 4352 | GGGTAACG GGCTAGCTACAACGA TGAAGGAA | 13101 |
| 7181 | CCUUCAGC G UUACCCAU | 4353 | ATGGGTAA GGCTAGCTACAACGA GCTGAAGG | 13102 |
| 7184 | UCAGCGUU A CCCAUAUG | 4354 | CATATGGG GGCTAGCTACAACGA AACGCTGA | 13103 |
| 7188 | CGUUACCC A UAUGGGCA | 4355 | TGCCCATA GGCTAGCTACAACGA GGGTAACG | 13104 |
| 7190 | UUACCCAU A UGGGCACG | 4356 | CGTGCCCA GGCTAGCTACAACGA ATGGGTAA | 13105 |
| 7194 | CCAUAUGG G CACGCCCG | 4357 | CGGGCGTG GGCTAGCTACAACGA CCATATGG | 13106 |
| 7196 | AUAUGGGC A CGCCCGGA | 4358 | TCCGGGCG GGCTAGCTACAACGA GCCCATAT | 13107 |
| 7198 | AUGGGCAC G CCCGGAUU | 4359 | AATCCGGG GGCTAGCTACAACGA GTGCCCAT | 13108 |
| 7204 | ACGCCCGG A UUACAACC | 4360 | GGTTGTAA GGCTAGCTACAACGA CCGGGCGT | 13109 |
| 7207 | CCCGGAUU A CAACCCUC | 4361 | GAGGGTTG GGCTAGCTACAACGA AATCCGGG | 13110 |
| 7210 | GGAUUACA A CCCUCCAC | 4362 | GTGGAGGG GGCTAGCTACAACGA TGTAATCC | 13111 |
| 7217 | AACCCUCC A CUACUAGA | 4363 | TCTAGTAG GGCTAGCTACAACGA GGAGGGTT | 13112 |
| 7220 | CCUCCACU A CUAGAGCC | 4364 | GGCTCTAG GGCTAGCTACAACGA AGTGGAGG | 13113 |
| 7226 | CUACUAGA G CCCUGGAA | 4365 | TTCCAGGG GGCTAGCTACAACGA TCTAGTAG | 13114 |
| 7237 | CUGGAAAG A CCCAGACU | 4366 | AGTCTGGG GGCTAGCTACAACGA CTTTCCAG | 13115 |
| 7243 | AGACCCAG A CUACGUCC | 4367 | GGACGTAG GGCTAGCTACAACGA CTGGGTCT | 13116 |
| 7246 | CCCAGACU A CGUCCCUC | 4368 | GAGGGACG GGCTAGCTACAACGA AGTCTGGG | 13117 |
| 7248 | CAGACUAC G UCCCUCCG | 4369 | CGGAGGGA GGCTAGCTACAACGA GTAGTCTG | 13118 |
| 7257 | UCCCUCCG G UGGUACAC | 4370 | GTGTACCA GGCTAGCTACAACGA CGGAGGGA | 13119 |
| 7260 | CUCCGGUG G UACACGGG | 4371 | CCCGTGTA GGCTAGCTACAACGA CACCGGAG | 13120 |
| 7262 | CCGGUGGU A CACGGGUG | 4372 | CACCCGTG GGCTAGCTACAACGA ACCACCGG | 13121 |
| 7264 | GGUGGUAC A CGGGUGCC | 4373 | GGCACCCG GGCTAGCTACAACGA GTACCACC | 13122 |
| 7268 | GUACACGG G UGCCCAUU | 4374 | AATGGGCA GGCTAGCTACAACGA CCGTGTAC | 13123 |
| 7270 | ACACGGGU G CCCAUUGC | 4375 | GCAATGGG GGCTAGCTACAACGA ACCCGTGT | 13124 |
| 7274 | GGGUGCCC A UUGCCACC | 4376 | GGTGGCAA GGCTAGCTACAACGA GGGCACCC | 13125 |
| 7277 | UGCCCAUU G CCACCUGC | 4377 | GCAGGTGG GGCTAGCTACAACGA AATGGGCA | 13126 |
| 7280 | CCAUUGCC A CCUGCCAA | 4378 | TTGGCAGG GGCTAGCTACAACGA GGCAATGG | 13127 |
| 7284 | UGCCACCU G CCAAGGCC | 4379 | GGCCTTGG GGCTAGCTACAACGA AGGTGGCA | 13128 |
| 7290 | CUGCCAAG G CCCCUCCA | 4380 | TGGAGGGG GGCTAGCTACAACGA CTTGGCAG | 13129 |
| 7299 | CCCCUCCA A UACCACCU | 4381 | AGGTGGTA GGCTAGCTACAACGA TGGAGGGG | 13130 |
| 7301 | CCUCCAAU A CCACCUCC | 4382 | GGAGGTGG GGCTAGCTACAACGA ATTGGAGG | 13131 |
| 7304 | CCAAUACC A CCUCCACG | 4383 | CGTGGAGG GGCTAGCTACAACGA GGTATTGG | 13132 |
| 7310 | CCACCUCC A CGGAGGAA | 4384 | TTCCTCCG GGCTAGCTACAACGA GGAGGTGG | 13133 |
| 7323 | GGAAGAGG A CGGUUGUU | 4385 | AACAACCG GGCTAGCTACAACGA CCTCTTCC | 13134 |
| 7326 | AGAGGACG G UUGUUCUG | 4386 | CAGAACAA GGCTAGCTACAACGA CGTCCTCT | 13135 |
| 7329 | GGACGGUU G UUCUGACA | 4387 | TGTCAGAA GGCTAGCTACAACGA AACCGTCC | 13136 |
| 7335 | UUGUUCUG A CAGAGUCC | 4388 | GGACTCTG GGCTAGCTACAACGA CAGAACAA | 13137 |
| 7340 | CUGACAGA G UCCACCGU | 4389 | ACGGTGGA GGCTAGCTACAACGA TCTGTCAG | 13138 |
| 7344 | CAGAGUCC A CCGUGUCU | 4390 | AGACACGG GGCTAGCTACAACGA GGACTCTG | 13139 |
| 7347 | AGUCCACC G UGUCUUCU | 4391 | AGAAGACA GGCTAGCTACAACGA GGTGGACT | 13140 |
| 7349 | UCCACCGU G UCUUCUGC | 4392 | GCAGAAGA GGCTAGCTACAACGA ACGGTGGA | 13141 |
| 7356 | UGUCUUCU G CCUUGGCG | 4393 | CGCCAAGG GGCTAGCTACAACGA AGAAGACA | 13142 |
| 7362 | CUGCCUUG G CGGAGCUC | 4394 | GAGCTCCG GGCTAGCTACAACGA CAAGGCAG | 13143 |
| 7367 | UUGGCGGA G CUCGCCAC | 4395 | GTGGCGAG GGCTAGCTACAACGA TCCGCCAA | 13144 |
| 7371 | CGGAGCUC G CCACAAAG | 4396 | CTTTGTGG GGCTAGCTACAACGA GAGCTCCG | 13145 |
| 7374 | AGCUCGCC A CAAAGACC | 4397 | GGTCTTTG GGCTAGCTACAACGA GGCGAGCT | 13146 |
| 7380 | CCACAAAG A CCUUCGGC | 4398 | GCCGAAGG GGCTAGCTACAACGA CTTTGTGG | 13147 |
| 7387 | GACCUUCG G CAGCUCUG | 4399 | CAGAGCTG GGCTAGCTACAACGA CGAAGGTC | 13148 |
| 7390 | CUUCGGCA G CUCUGAAU | 4400 | ATTCAGAG GGCTAGCTACAACGA TGCCGAAG | 13149 |
| 7397 | AGCUCUGA A UCAUCGGC | 4401 | GCCGATGA GGCTAGCTACAACGA TCAGAGCT | 13150 |
| 7400 | UCUGAAUC A UCGGCCGC | 4402 | GCGGCCGA GGCTAGCTACAACGA GATTCAGA | 13151 |
| 7404 | AAUCAUCG G CCGCUGAU | 4403 | ATCAGCGG GGCTAGCTACAACGA CGATGATT | 13152 |
| 7407 | CAUCGGCC G CUGAUAGA | 4404 | TCTATCAG GGCTAGCTACAACGA GGCCGATG | 13153 |
| 7411 | GGCCGCUG A UAGAGGUA | 4405 | TACCTCTA GGCTAGCTACAACGA CAGCGGCC | 13154 |
| 7417 | UGAUAGAG G UACGGCAA | 4406 | TTGCCGTA GGCTAGCTACAACGA CTCTATCA | 13155 |
| 7419 | AUAGAGGU A CGGCAACC | 4407 | GGTTGCCG GGCTAGCTACAACGA ACCTCTAT | 13156 |
| 7422 | GAGGUACG G CAACCGCC | 4408 | GGCGGTTG GGCTAGCTACAACGA CGTACCTC | 13157 |
| 7425 | GUACGGCA A CCGCCCCC | 4409 | GGGGGCGG GGCTAGCTACAACGA TGCCGTAC | 13158 |
| 7428 | CGGCAACC G CCCCCCCC | 4410 | GGGGGGGG GGCTAGCTACAACGA GGTTGCCG | 13159 |
| 7438 | CCCCCCCG A CCAGACCU | 4411 | AGGTCTGG GGCTAGCTACAACGA CGGGGGGG | 13160 |
| 7443 | CCGACCAG A CCUCCAAU | 4412 | ATTGGAGG GGCTAGCTACAACGA CTGGTCGG | 13161 |
| 7450 | GACCUCCA A UGACGGUG | 4413 | CACCGTCA GGCTAGCTACAACGA TGGAGGTC | 13162 |
| 7453 | CUCCAAUG A CGGUGACG | 4414 | CGTCACCG GGCTAGCTACAACGA CATTGGAG | 13163 |
| 7456 | CAAUGACG G UGACGCAG | 4415 | CTGCGTCA GGCTAGCTACAACGA CGTCATTG | 13164 |
| 7459 | UGACGGUG A CGCAGGAU | 4416 | ATCCTGCG GGCTAGCTACAACGA CACCGTCA | 13165 |
| 7461 | ACGGUGAC G CAGGAUCC | 4417 | GGATCCTG GGCTAGCTACAACGA GTCACCGT | 13166 |
| 7466 | GACGCAGG A UCCGACGU | 4418 | ACGTCGGA GGCTAGCTACAACGA CCTGCGTC | 13167 |
| 7471 | AGGAUCCG A CGUUGAGU | 4419 | ACTCAACG GGCTAGCTACAACGA CGGATCCT | 13168 |
| 7473 | GAUCCGAC G UUGAGUCG | 4420 | CGACTCAA GGCTAGCTACAACGA GTCGGATC | 13169 |
| 7478 | GACGUUGA G UCGUACUC | 4421 | GAGTACGA GGCTAGCTACAACGA TCAACGTC | 13170 |
| 7481 | GUUGAGUC G UACUCCUC | 4422 | GAGGAGTA GGCTAGCTACAACGA GACTCAAC | 13171 |
| 7483 | UGAGUCGU A CUCCUCUA | 4423 | TAGAGGAG GGCTAGCTACAACGA ACGACTCA | 13172 |
| 7491 | ACUCCUCU A UGCCCCCC | 4424 | GGGGGGCA GGCTAGCTACAACGA AGAGGAGT | 13173 |
| 7493 | UCCUCUAU G CCCCCCCU | 4425 | AGGGGGGG GGCTAGCTACAACGA ATAGAGGA | 13174 |
| 7511 | GAGGGGGA G CCGGGGGA | 4426 | TCCCCCGG GGCTAGCTACAACGA TCCCCCTC | 13175 |
| 7519 | GCCGGGGG A UCCCGAUC | 4427 | GATCGGGA GGCTAGCTACAACGA CCCCCGGC | 13176 |
| 7525 | GGAUCCCG A UCUCAGCG | 4428 | CGCTGAGA GGCTAGCTACAACGA CGGGATCC | 13177 |
| 7531 | CGAUCUCA G CGACGGGU | 4429 | ACCCGTCG GGCTAGCTACAACGA TGAGATCG | 13178 |
| 7534 | UCUCAGCG A CGGGUCUU | 4430 | AAGACCCG GGCTAGCTACAACGA CGCTGAGA | 13179 |
| 7538 | AGCGACGG G UCUUGGUC | 4431 | GACCAAGA GGCTAGCTACAACGA CCGTCGCT | 13180 |
| 7544 | GGGUCUUG G UCUACCGU | 4432 | ACGGTAGA GGCTAGCTACAACGA CAAGACCC | 13181 |
| 7548 | CUUGGUCU A CCGUGAGC | 4433 | GCTCACGG GGCTAGCTACAACGA AGACCAAG | 13182 |
| 7551 | GGUCUACC G UGAGCGAA | 4434 | TTCGCTCA GGCTAGCTACAACGA GGTAGACC | 13183 |
| 7555 | UACCGUGA G CGAAGAGG | 4435 | CCTCTTCG GGCTAGCTACAACGA TCACGGTA | 13184 |
| 7563 | GCGAAGAG G CUGGCGAG | 4436 | CTCGCCAG GGCTAGCTACAACGA CTCTTCGC | 13185 |
| 7567 | AGAGGCUG G CGAGGAUG | 4437 | CATCCTCG GGCTAGCTACAACGA CAGCCTCT | 13186 |
| 7573 | UGGCGAGG A UGUCGUCU | 4438 | AGACGACA GGCTAGCTACAACGA CCTCGCCA | 13187 |
| 7575 | GCGAGGAU G UCGUCUGC | 4439 | GCAGACGA GGCTAGCTACAACGA ATCCTCGC | 13188 |
| 7578 | AGGAUGUC G UCUGCUGC | 4440 | GCAGCAGA GGCTAGCTACAACGA GACATCCT | 13189 |
| 7582 | UGUCGUCU G CUGCUCGA | 4441 | TCGAGCAG GGCTAGCTACAACGA ACACGACA | 13190 |
| 7585 | CGUCUGCU G CUCGAUGU | 4442 | ACATCGAG GGCTAGCTACAACGA AGCAGACG | 13191 |
| 7590 | GCUGCUCG A UGUCCUAC | 4443 | GTAGGACA GGCTAGCTACAACGA CGAGCAGC | 13192 |
| 7592 | UGCUCGAU G UCCUACAC | 4444 | GTGTAGGA GGCTAGCTACAACGA ATCGAGCA | 13193 |
| 7597 | GAUGUCCU A CACAUGGA | 4445 | TCCATGTG GGCTAGCTACAACGA AGGACATC | 13194 |
| 7599 | UGUCCUAC A CAUGGACG | 4446 | CGTCCATG GGCTAGCTACAACGA GTAGGACA | 13195 |
| 7601 | UCCUACAC A UGGACGGG | 4447 | CCCGTCCA GGCTAGCTACAACGA GTGTAGGA | 13196 |
| 7605 | ACACAUGG A CGGGCGCC | 4448 | GGCGCCCG GGCTAGCTACAACGA CCATGTGT | 13197 |
| 7609 | AUGGACGG G CGCCCUGA | 4449 | TCAGGGCG GGCTAGCTACAACGA CCGTCCAT | 13198 |
| 7611 | GGACGGGC G CCCUGAUC | 4450 | GATCAGGG GGCTAGCTACAACGA GCCCGTCC | 13199 |
| 7617 | GCGCCCUG A UCACGCCA | 4451 | TGGCGTGA GGCTAGCTACAACGA CAGGGCGC | 13200 |
| 7620 | CCCUGAUC A CGCCAUGC | 4452 | GCATGGCG GGCTAGCTACAACGA GATCAGGG | 13201 |
| 7622 | CUGAUCAC G CCAUGCGC | 4453 | GCGCATGG GGCTAGCTACAACGA GTGATCAG | 13202 |
| 7625 | AUCACGCC A UGCGCUGC | 4454 | GCAGCGCA GGCTAGCTACAACGA GGCGTGAT | 13203 |
| 7627 | CACGCCAU G CGCUGCGG | 4455 | CCGCAGCG GGCTAGCTACAACGA ATGGCGTG | 13204 |
| 7629 | CGCCAUGC G CUGCGGAG | 4456 | CTCCGCAG GGCTAGCTACAACGA GCATGGCG | 13205 |
| 7632 | CAUGCGCU G CGGAGGAA | 4457 | TTCCTCCG GGCTAGCTACAACGA AGCGCATG | 13206 |
| 7642 | GGAGGAAA G CAAGUUGC | 4458 | GCAACTTG GGCTAGCTACAACGA TTTCCTCC | 13207 |
| 7646 | GAAAGCAA G UUGCCCAU | 4459 | ATGGGCAA GGCTAGCTACAACGA TTGCTTTC | 13208 |
| 7649 | AGCAAGUU G CCCAUCAA | 4460 | TTGATGGG GGCTAGCTACAACGA AACTTGCT | 13209 |
| 7653 | ACUUGCCC A UCAACGCG | 4461 | CGCGTTGA GGCTAGCTACAACGA GGGCAACT | 13210 |
| 7657 | GCCCAUCA A CGCGUUGA | 4462 | TCAACGCG GGCTAGCTACAACCA TGATGCGC | 13211 |
| 7659 | CCAUCAAC G CGUUGAGC | 4463 | GCTCAACG GGCTAGCTACAACGA GTTGATGG | 13212 |
| 7661 | AUCAACGC G UUGAGCAA | 4464 | TTGCTCAA GGCTAGCTACAACGA GCGTTGAT | 13213 |
| 7666 | CGCGUUGA G CAACUCUU | 4465 | AAGAGTTG GGCTAGCTACAACGA TCAACGCG | 13214 |
| 7669 | GUUGAGCA A CUCUUUGC | 4466 | GCAAAGAG GGCTAGCTACAACGA TGCTCAAC | 13215 |
| 7676 | AACUCUUU G CUGCGUCA | 4467 | TGACGCAG GGCTAGCTACAACGA AAAGAGTT | 13216 |
| 7679 | UCUUUGCU G CGUCACCA | 4468 | TGGTGACG GGCTAGCTACAACGA AGCAAAGA | 13217 |
| 7681 | UUUGCUGC G UCACCACA | 4469 | TGTGGTGA GGCTAGCTACAACGA GCAGCAAA | 13218 |
| 7684 | GCUGCGUC A CCACAACA | 4470 | TGTTGTGG GGCTAGCTACAACGA GACGCAGC | 13219 |
| 7687 | GCGUCACC A CAACAUGG | 4471 | CCATGTTG GGCTAGCTACAACGA GGTGACGC | 13220 |
| 7690 | UCACCACA A CAUGGUCU | 4472 | AGACCATG GGCTAGCTACAACGA TGTGGTGA | 13221 |
| 7692 | ACCACAAC A UGGUCUAC | 4473 | GTAGACCA GGCTAGCTACAACGA GTTGTGGT | 13222 |
| 7695 | ACAACAUG G UCUACGCU | 4474 | AGCGTAGA GGCTAGCTACAACGA CATGTTGT | 13223 |
| 7699 | CAUGGUCU A CGCUACAA | 4475 | TTGTAGCG GGCTAGCTACAACGA AGACCATG | 13224 |
| 7701 | UGGUCUAC G CUACAACA | 4476 | TGTTGTAG GGCTAGCTACAACGA GTAGACCA | 13225 |
| 7704 | UCUACGCU A CAACAUCU | 4477 | AGATGTTG GGCTAGCTACAACGA AGCGTAGA | 13226 |
| 7707 | ACGCUACA A CAUCUCGC | 4478 | GCGAGATG GGCTAGCTACAACGA TGTAGCGT | 13227 |
| 7709 | GCUACAAC A UCUCGCAG | 4479 | CTGCGAGA GGCTAGCTACAACGA GTTGTAGC | 13228 |
| 7714 | AACAUCUC G CAGCGCAA | 4480 | TTGCGCTG GGCTAGCTACAACGA GAGATGTT | 13229 |
| 7717 | AUCUCGCA G CGCAAGCC | 4481 | GGCTTGCG GGCTAGCTACAACGA TGCGAGAT | 13230 |
| 7719 | CUCGCAGC G CAAGCCAG | 4482 | CTGGCTTG GGCTAGCTACAACGA GCTGCGAG | 13231 |
| 7723 | CAGCGCAA G CCAGCGGC | 4483 | GCCGCTGG GGCTAGCTACAACGA TTGCGCTG | 13232 |
| 7727 | GCAAGCCA G CGGCAGAA | 4484 | TTCTGCCG GGCTAGCTACAACGA TGGCTTGC | 13233 |
| 7730 | AGCCAGCG G CAGAAGAA | 4485 | TTCTTCTG GGCTAGCTACAACGA CGCTGGCT | 13234 |
| 7740 | AGAAGAAG G UCACCUUU | 4486 | AAAGGTGA GGCTAGCTACAACGA CTTCTTCT | 13235 |
| 7743 | AGAAGGUC A CCUUUGAC | 4487 | GTCAAAGG GGCTAGCTACAACGA GACCTTCT | 13236 |
| 7750 | CACCUUUG A CAGACUGC | 4488 | GCAGTCTG GGCTAGCTACAACGA CAAAGGTG | 13237 |
| 7754 | UUUGACAG A CUGCAAGU | 4489 | ACTTGCAG GGCTAGCTACAACGA CTGTCAAA | 13238 |
| 7757 | GACAGACU G CAAGUCCU | 4490 | AGGACTTG GGCTAGCTACAACGA AGTCTGTC | 13239 |
| 7761 | GACUGCAA G UCCUGGAC | 4491 | GTCCAGGA GGCTAGCTACAACGA TTGCAGTC | 13240 |
| 7768 | AGUCCUGG A CGACCACU | 4492 | AGTGGTCG GGCTAGCTACAACGA CCAGGACT | 13241 |
| 7771 | CCUGGACG A CCACUACC | 4493 | GGTAGTGG GGCTAGCTACAACGA CGTCCAGG | 13242 |
| 7774 | GGACGACC A CUACCGGG | 4494 | CCCGGTAG GGCTAGCTACAACGA GGTCGTCC | 13243 |
| 7777 | CGACCACU A CCGGGACG | 4495 | CGTCCCGG GGCTAGCTACAACGA AGTGGTCG | 13244 |
| 7783 | CUACCGGG A CGUGCUCA | 4496 | TGAGCACG GGCTAGCTACAACGA CCCGGTAG | 13245 |
| 7785 | ACCGGGAC G UGCUCAAG | 4497 | CTTGAGCA GGCTAGCTACAACGA GTCCCGGT | 13246 |
| 7787 | CGGGACGU G CUCAAGGA | 4498 | TCCTTGAG GGCTAGCTACAACGA ACGTCCCG | 13247 |
| 7797 | UCAAGGAG A UGAAGGCG | 4499 | CGCCTTCA GGCTAGCTACAACGA CTCCTTGA | 13248 |
| 7803 | AGAUGAAG G CGAAGGCG | 4500 | CGCCTTCG GGCTAGCTACAACGA CTTCATCT | 13249 |
| 7809 | AGGCGAAG G CGUCCACA | 4501 | TGTGGACG GGCTAGCTACAACGA CTTCGCCT | 13250 |
| 7811 | GCGAAGGC G UCCACAGU | 4502 | ACTGTGGA GGCTAGCTACAACGA GCCTTCGC | 13251 |
| 7815 | AGGCGUCC A CACUUAAG | 4503 | CTTAACTG GGCTAGCTACAACGA GGACGCCT | 13252 |
| 7818 | CGUCCACA G UUAAGGCU | 4504 | AGCCTTAA GGCTAGCTACAACGA TGTGGACG | 13253 |
| 7824 | CACUUAAG G CUAAACUU | 4505 | AAGTTTAG GGCTAGCTACAACGA CTTAACTG | 13254 |
| 7829 | AAGGCUAA A CUUCUAUC | 4506 | GATAGAAG GGCTAGCTACAACGA TTAGCCTT | 13255 |
| 7835 | AAACUUCU A UCCGUAGA | 4507 | TCTACGGA GGCTAGCTACAACGA AGAAGTTT | 13256 |
| 7839 | UUCUAUCC G UAGAGGAA | 4508 | TTCCTCTA GGCTAGCTACAACGA GGATAGAA | 13257 |
| 7848 | UAGAGGAA G CCUGCAGA | 4509 | TCTGCAGG GGCTAGCTACAACGA TTCCTCTA | 13258 |
| 7852 | GGAAGCCU G CAGACUGA | 4510 | TCAGTCTG GGCTAGCTACAACGA AGGCTTCC | 13259 |
| 7856 | GCCUCCAG A CUGACGCC | 4511 | GGCGTCAG GGCTAGCTACAACGA CTGCAGGC | 13260 |
| 7860 | GCAGACUG A CGCCCCCA | 4512 | TGGGGGCG GGCTAGCTACAACGA CAGTCTGC | 13261 |
| 7862 | AGACUGAC G CCCCCACA | 4513 | TGTGGGGG GGCTAGCTACAACGA GTCAGTCT | 13262 |
| 7868 | ACGCCCCC A CAUUCGGC | 4514 | GCCGAATG GGCTAGCTACAACGA CGGGGCGT | 13263 |
| 7870 | GCCCCCAC A UUCGGCCA | 4515 | TGGCCGAA GGCTAGCTACAACGA GTGGGGGC | 13264 |
| 7875 | CACAUUCG G CCAGGUCC | 4516 | GGACCTGG GGCTAGCTACAACGA CGAATGTG | 13265 |
| 7880 | UCGGCCAG G UCCAAAUU | 4517 | AATTTGGA GGCTAGCTACAACGA CTGGCCGA | 13266 |
| 7886 | AGGUCCAA A UUUGGUUA | 4518 | TAACCAAA GGCTAGCTACAACGA TTGGACCT | 13267 |
| 7891 | CAAAUUUG G UUAUGGGG | 4519 | CCCCATAA GGCTAGCTACAACGA CAAATTTG | 13268 |
| 7894 | AUUUGGUU A UGGGGCAA | 4520 | TTGCCCCA GGCTAGCTACAACGA AACCAAAT | 13269 |
| 7899 | GUUAUGGG G CAAAGGAC | 4521 | GTCCTTTG GGCTAGCTACAACGA CCCATAAC | 13270 |
| 7906 | GGCAAAGG A CGUCCGGA | 4522 | TCCGGACG GGCTAGCTACAACGA CCTTTGCC | 13271 |
| 7908 | CAAAGGAC G UCCGGAAC | 4523 | GTTCCGGA GGCTAGCTACAACGA GTCCTTTG | 13272 |
| 7915 | CGUCCGGA A CCUAUCCA | 4524 | TGGATAGG GGCTAGCTACAACGA TCCGGACG | 13273 |
| 7919 | CGGAACCU A UCCAGCGG | 4525 | CCGCTGGA GGCTAGCTACAACGA AGGTTCCG | 13274 |
| 7924 | CCUAUCCA G CGGGGCCG | 4526 | CGGCCCCG GGCTAGCTACAACGA TGGATAGG | 13275 |
| 7929 | CCAGCGGG G CCGUCAAC | 4527 | GTTGACGG GGCTAGCTACAACGA CCCGCTGG | 13276 |
| 7932 | GCGGGGCC G UCAACCAC | 4528 | GTGGTTGA GGCTAGCTACAACGA GGCCCCGC | 13277 |
| 7936 | GGCCGUCA A CCACAUCC | 4529 | GGATGTGG GGCTAGCTACAACGA TGACGGCC | 13278 |
| 7939 | CGUCAACC A CAUCCGCU | 4530 | AGCGGATG GGCTAGCTACAACGA GGTTGACG | 13279 |
| 7941 | UCAACCAC A UCCGCUCC | 4531 | GGAGCGGA GGCTAGCTACAACGA GTGGTTGA | 13280 |
| 7945 | CCACAUCC G CUCCGUGU | 4532 | ACACGGAG GGCTAGCTACAACGA GGATGTGG | 13281 |
| 7950 | UCCGCUCC G UGUGGAAG | 4533 | CTTCCACA GGCTAGCTACAACGA GGAGCGGA | 13282 |
| 7952 | CGCUCCGU G UGGAAGGA | 4534 | TCCTTCCA GGCTAGCTACAACGA ACGGAGCG | 13283 |
| 7960 | GUGGAAGG A CUUGCUGG | 4535 | CCAGCAAG GGCTAGCTACAACGA CCTTCCAC | 13284 |
| 7964 | AAGGACUU G CUGGAAGA | 4536 | TCTTCCAG GGCTAGCTACAACGA AAGTCCTT | 13285 |
| 7972 | GCUGGAAG A CACUGAGA | 4537 | TCTCAGTG GGCTAGCTACAACGA CTTCCAGC | 13286 |
| 7974 | UGGAAGAC A CUGAGACA | 4538 | TGTCTCAG GGCTAGCTACAACGA GTCTTCCA | 13287 |
| 7980 | ACACUGAG A CACCAAUU | 4539 | AATTGGTG GGCTAGCTACAACGA CTCAGTGT | 13288 |
| 7982 | ACUGAGAC A CCAAUUGA | 4540 | TCAATTGG GGCTAGCTACAACGA GTCTCAGT | 13289 |
| 7986 | AGACACCA A UUGAUACC | 4541 | GGTATCAA GGCTAGCTACAACGA TGGTGTCT | 13290 |
| 7990 | ACCAAUUG A UACCACCA | 4542 | TGGTGGTA GGCTAGCTACAACGA CAATTGGT | 13291 |
| 7992 | CAAUUGAU A CCACCAUC | 4543 | GATGGTGG GGCTAGCTACAACGA ATCAATTG | 13292 |
| 7995 | UUGAUACC A CCAUCAUG | 4544 | CATGATGG GGCTAGCTACAACGA GGTATCAA | 13293 |
| 7998 | AUACCACC A UCAUGGCA | 4545 | TGCCATGA GGCTAGCTACAACGA GGTGGTAT | 13294 |
| 8001 | CCACCAUC A UGGCAAAA | 4546 | TTTTGCCA GGCTAGCTACAACGA GATGGTGG | 13295 |
| 8004 | CCAUCAUG G CAAAAAAU | 4547 | ATTTTTTG GGCTAGCTACAACGA CATGATGG | 13296 |
| 8011 | GGCAAAAA A UGAGGUUU | 4548 | AAACCTCA GGCTAGCTACAACGA TTTTTGCC | 13297 |
| 8016 | AAAAUGAG G UUUUCUGC | 4549 | GCAGAAAA GGCTAGCTACAACGA CTCATTTT | 13298 |
| 8023 | GGUUUUCU G CGUCCAAC | 4550 | GTTGGACG GGCTAGCTACAACGA AGAAAACC | 13299 |
| 8025 | UUUUCUGC G UCCAACCA | 4551 | TGGTTGGA GGCTAGCTACAACGA GCAGAAAA | 13300 |
| 8030 | UGCGUCCA A CCAGAGAA | 4552 | TTCTCTGG GGCTAGCTACAACGA TGGACGCA | 13301 |
| 8044 | GAAAGGAG G CCGCAAGC | 4553 | GCTTGCGG GGCTAGCTACAACGA CTCCTTTC | 13302 |
| 8047 | AGGAGGCC G CAAGCCAG | 4554 | CTGGCTTG GGCTAGCTACAACGA GGCCTCCT | 13303 |
| 8051 | GGCCGCAA G CCAGCUCG | 4555 | CGAGCTGG GGCTAGCTACAACGA TTGCGGCC | 13304 |
| 8055 | GCAAGCCA G CUCGCCUU | 4556 | AAGGCGAG GGCTAGCTACAACGA TGGCTTGC | 13305 |
| 8059 | GCCAGCUC G CCUUAUCG | 4557 | CGATAAGG GGCTAGCTACAACGA GAGCTGGC | 13306 |
| 8064 | CUCGCCUU A UCGUGUUC | 4558 | GAACACGA GGCTAGCTACAACGA AAGGCGAG | 13307 |
| 8067 | GCCUUAUC G UGUUCCCA | 4559 | TGGGAACA GGCTAGCTACAACGA GATAAGGC | 13308 |
| 8069 | CUUAUCGU G UUCCCAGA | 4560 | TCTGCGAA GGCTAGCTACAACGA ACGATAAG | 13309 |
| 8077 | GUUCCCAG A CUUGGGGG | 4561 | CCCCCAAG GGCTAGCTACAACGA CTGGGAAC | 13310 |
| 8085 | ACUUGGGG G UUCGUGUG | 4562 | CACACGAA GGCTAGCTACAACGA CCCCAAGT | 13311 |
| 8089 | GGGGGUUC G UGUGUGCG | 4563 | CGCACACA GGCTAGCTACAACGA GAACCCCC | 13312 |
| 8091 | GGGUUCGU G UGUGCGAG | 4564 | CTCGCACA GGCTAGCTACAACGA ACGAACCC | 13313 |
| 8093 | GUUCGUGU G UGCGAGAA | 4565 | TTCTCGCA GGCTAGCTACAACGA ACACGAAC | 13314 |
| 8095 | UCGUGUGU G CGAGAAAA | 4566 | TTTTCTCG GGCTAGCTACAACGA ACACACGA | 13315 |
| 8103 | GCGAGAAA A UGGCCCUU | 4567 | AAGGGCCA GGCTAGCTACAACGA TTTCTCGC | 13316 |
| 8106 | AGAAAAUG G CCCUUUAC | 4568 | GTAAAGGG GGCTAGCTACAACGA CATTTTCT | 13317 |
| 8113 | GGCCCUUU A CGACGUGG | 4569 | CCACGTCG GGCTAGCTACAACGA AAAGGGCC | 13318 |
| 8116 | CCUUUACG A CGUGGUCU | 4570 | AGACCACG GGCTAGCTACAACGA CGTAAAGG | 13319 |
| 8118 | UUUACGAC G UGGUCUCC | 4571 | GGAGACCA GGCTAGCTACAACGA GTCGTAAA | 13320 |
| 8121 | ACGACGUG G UCUCCACC | 4572 | GGTGGAGA GGCTAGCTACAACGA CACGTCGT | 13321 |
| 8127 | UGGUCUCC A CCCUUCCU | 4573 | AGGAAGGG GGCTAGCTACAACGA GGAGACCA | 13322 |
| 8139 | UUCCUCAG G CCGUGAUG | 4574 | CATCACGG GGCTAGCTACAACGA CTGAGGAA | 13323 |
| 8142 | CUCAGGCC G UGAUGGGC | 4575 | GCCCATCA GGCTAGCTACAACGA GGCCTGAG | 13324 |
| 8145 | AGGCCGUG A UGGGCUCU | 4576 | AGAGCCCA GGCTAGCTACAACGA CACGGCCT | 13325 |
| 8149 | CGUGAUGG G CUCUUCAU | 4577 | ATGAAGAG GGCTAGCTACAACGA CCATCACG | 13326 |
| 8156 | GGCUCUUC A UACCGAUU | 4578 | AATCCGTA GGCTAGCTACAACGA GAAGAGCC | 13327 |
| 8158 | CUCUUCAU A CGGAUUCC | 4579 | GGAATCCG GGCTAGCTACAACGA ATGAAGAG | 13328 |
| 8162 | UCAUACGG A UUCCAGUA | 4580 | TACTGGAA GGCTAGCTACAACGA CCGTATGA | 13329 |
| 8168 | GGAUUCCA G UACUCUCC | 4581 | GGAGAGTA GGCTAGCTACAACGA TGGAATCC | 13330 |
| 8170 | AUUCCAGU A CUCUCCUG | 4582 | CAGGAGAG GGCTAGCTACAACGA ACTGGAAT | 13331 |
| 8180 | UCUCCUGG G CAGCGGGU | 4583 | ACCCGCTG GGCTAGCTACAACGA CCAGGAGA | 13332 |
| 8183 | CCUGGGCA G CGGGUUGA | 4584 | TCAACCCG GGCTAGCTACAACGA TGCCCAGG | 13333 |
| 8187 | GGCAGCGG G UUGAGUUC | 4585 | GAACTCAA GGCTAGCTACAACGA CCGCTGCC | 13334 |
| 8192 | CGGGUUGA G UUCCUGGU | 4586 | ACCAGGAA GGCTAGCTACAACGA TCAACCCG | 13335 |
| 8199 | AGUUCCUG G UGAAUGCC | 4587 | GGCATTCA GGCTAGCTACAACGA CAGGAACT | 13336 |
| 8203 | CCUGGUGA A UGCCUGGA | 4588 | TCCAGGCA GGCTAGCTACAACGA TCACCAGG | 13337 |
| 8205 | UGGUGAAU G CCUGGAAA | 4589 | TTTCCAGG GGCTAGCTACAACGA ATTCACCA | 13338 |
| 8213 | GCCUGGAA A UCAAAGAA | 4590 | TTCTTTGA GGCTAGCTACAACGA TTCCAGGC | 13339 |
| 8222 | UCAAAGAA A UGCCCUAU | 4591 | ATAGGGCA GGCTAGCTACAACGA TTCTTTGA | 13340 |
| 8224 | AAAGAAAU G CCCUAUGG | 4592 | CCATAGGG GGCTAGCTACAACGA ATTTCTTT | 13341 |
| 8229 | AAUGCCCU A UGGGCUUU | 4593 | AAAGCCCA GGCTAGCTACAACGA AGGGCATT | 13342 |
| 8233 | CCCUAUGG G CUUUGCAU | 4594 | ATGCAAAG GGCTAGCTACAACGA CCATAGGG | 13343 |
| 8238 | UGGGCUUU G CAUAUGAC | 4595 | GTCATATG GGCTAGCTACAACGA AAAGCCCA | 13344 |
| 8240 | GGCUUUGC A UAUGACAC | 4596 | GTGTCATA GGCTAGCTACAACGA GCAAAGCC | 13345 |
| 8242 | CUUUGCAU A UGACACCC | 4597 | GGGTGTCA GGCTAGCTACAACGA ATGCAAAG | 13346 |
| 8245 | UGCAUAUG A CACCCGCU | 4598 | AGCGGGTG GGCTAGCTACAACGA CATATGCA | 13347 |
| 8247 | CAUAUGAC A CCCGCUGU | 4599 | ACAGCGGG GGCTAGCTACAACGA GTCATATG | 13348 |
| 8251 | UGACACCC G CUGUUUCG | 4600 | CGAAACAG GGCTAGCTACAACGA GGGTGTCA | 13349 |
| 8254 | CACCCGCU G UUUCGACU | 4601 | AGTCGAAA GGCTAGCTACAACGA AGCGGGTG | 13350 |
| 8260 | CUGUUUCG A CUCAACAG | 4602 | CTGTTGAG GGCTAGCTACAACGA CGAAACAG | 13351 |
| 8265 | UCGACUCA A CAGUCACC | 4603 | GGTGACTG GGCTAGCTACAACGA TGAGTCGA | 13352 |
| 8268 | ACUCAACA G UCACCGAG | 4604 | CTCGGTGA GGCTAGCTACAACGA TGTTGAGT | 13353 |
| 8271 | CAACAGUC A CCGAGAGU | 4605 | ACTCTCGG GGCTAGCTACAACGA GACTGTTG | 13354 |
| 8278 | CACCGAGA G UGACAUCC | 4606 | GGATGTCA GGCTAGCTACAACGA TCTCGGTG | 13355 |
| 8281 | CGAGAGUG A CAUCCGUG | 4607 | CACGGATG GGCTAGCTACAACGA CACTCTCG | 13356 |
| 8283 | AGAGUGAC A UCCGUGUC | 4608 | GACACGGA GGCTAGCTACAACGA GTCACTCT | 13357 |
| 8287 | UGACAUCC G UGUCGAGG | 4609 | CCTCGACA GGCTAGCTACAACGA GGATGTCA | 13358 |
| 8289 | ACAUCCGU G UCCAGGAG | 4610 | CTCCTCGA GGCTAGCTACAACGA ACGGATGT | 13359 |
| 8297 | GUCGAGGA G UCAAUUUA | 4611 | TAAATTGA GGCTAGCTACAACGA TCCTCGAC | 13360 |
| 8301 | AGGAGUCA A UUUACCAA | 4612 | TTGGTAAA GGCTAGCTACAACGA TGACTCCT | 13361 |
| 8305 | GUCAAUUU A CCAAUGUU | 4613 | AACATTGG GGCTAGCTACAACGA AAATTGAC | 13362 |
| 8309 | AUUUACCA A UGUUGUGA | 4614 | TCACAACA GGCTAGCTACAACGA TGGTAAAT | 13363 |
| 8311 | UUACCAAU G UUGUGACU | 4615 | AGTCACAA GGCTAGCTACAACGA ATTGGTAA | 13364 |
| 8314 | CCAAUGUU G UGACUUGG | 4616 | CCAAGTCA GGCTAGCTACAACGA AACATTGG | 13365 |
| 8317 | AUGUUGUG A CUUGGCCC | 4617 | GGGCCAAG GGCTAGCTACAACGA CACAACAT | 13366 |
| 8322 | GUGACUUG G CCCCCGAA | 4618 | TTCGGGGG GGCTAGCTACAACGA CAAGTCAC | 13367 |
| 8331 | CCCCCGAA G CCAGACAG | 4619 | CTGTCTGG GGCTAGCTACAACGA TTCGGGGG | 13368 |
| 8336 | GAAGCCAG A CAGGCCAU | 4620 | ATGGCCTG GGCTAGCTACAACGA CTGGCTTC | 13369 |
| 8340 | CCAGACAG G CCAUAAGG | 4621 | CCTTATGG GGCTAGCTACAACGA CTGTCTGG | 13370 |
| 8343 | GACAGGCC A UAAGGUCG | 4622 | CGACCTTA GGCTAGCTACAACGA GGCCTGTC | 13371 |
| 8348 | GCCAUAAG G UCGCUCAC | 4623 | GTGAGCGA GGCTAGCTACAACGA CTTATGGC | 13372 |
| 8351 | AUAAGGUC G CUCACAGA | 4624 | TCTGTGAG GGCTAGCTACAACGA GACCTTAT | 13373 |
| 8355 | GGUCGCUC A CAGAGCGG | 4625 | CCGCTCTG GGCTAGCTACAACGA GAGCGACC | 13374 |
| 8360 | CUCACAGA G CGGCUUUA | 4626 | TAAAGCCG GGCTAGCTACAACGA TCTGTGAG | 13375 |
| 8363 | ACAGAGCG G CUUUAUAU | 4627 | ATATAAAG GGCTAGCTACAACGA CGCTCTGT | 13376 |
| 8368 | GCGGCUUU A UAUCGGGG | 4628 | CCCCGATA GGCTAGCTACAACGA AAAGCCGC | 13377 |
| 8370 | GGCUUUAU A UCGGGGGU | 4629 | ACCCCCGA GGCTAGCTACAACGA ATAAAGCC | 13378 |
| 8377 | UAUCGGGG G UCCUCUGA | 4630 | TCAGAGGA GGCTAGCTACAACGA CCCCGATA | 13379 |
| 8385 | GUCCUCUG A CUAAUUCA | 4631 | TGAATTAG GGCTAGCTACAACGA CAGAGGAC | 13380 |
| 8389 | UCUGACUA A UUCAAAAG | 4632 | CTTTTGAA GGCTAGCTACAACGA TAGTCAGA | 13381 |
| 8399 | UCAAAAGG G CAGAACUG | 4633 | CAGTTCTG GGCTAGCTACAACGA CCTTTTGA | 13382 |
| 8404 | AGGGCAGA A CUGCGGUU | 4634 | AACCGCAG GGCTAGCTACAACGA TCTGCCCT | 13383 |
| 8407 | GCAGAACU G CGGUUAUC | 4635 | GATAACCG GGCTAGCTACAACGA AGTTCTGC | 13384 |
| 8410 | GAACUGCG G UUAUCGCC | 4636 | GGCGATAA GGCTAGCTACAACGA CGCAGTTC | 13385 |
| 8413 | CUGCGGUU A UCGCCGGU | 4637 | ACCGGCGA GGCTAGCTACAACGA AACCGCAG | 13386 |
| 8416 | CGGUUAUC G CCGGUGCC | 4638 | GGCACCGG GGCTAGCTACAACGA GATAACCG | 13387 |
| 8420 | UAUCGCCG G UGCCGCGC | 4639 | GCGCGGCA GGCTAGCTACAACGA CGGCGATA | 13388 |
| 8422 | UCGCCGGU G CCGCGCGA | 4640 | TCGCGCGG GGCTAGCTACAACGA ACCGGCGA | 13389 |
| 8425 | CCGGUGCC G CGCGAGCG | 4641 | CGCTCGCG GGCTAGCTACAACGA GGCACCGG | 13390 |
| 8427 | GGUGCCGC G CGAGCGGC | 4642 | GCCGCTCG GGCTAGCTACAACGA GCGGCACC | 13391 |
| 8431 | CCGCGCGA G CGGCGUGC | 4643 | GCACGCCG GGCTAGCTACAACGA TCGCGCGG | 13392 |
| 8434 | CGCGAGCG G CGUGCUGA | 4644 | TCAGCACG GGCTAGCTACAACGA CGCTCGCG | 13393 |
| 8436 | CGAGCGGC G UGCUGACG | 4645 | CGTCAGCA GGCTAGCTACAACGA GCCGCTCG | 13394 |
| 8438 | AGCGGCGU G CUGACGAC | 4646 | GTCGTCAG GGCTAGCTACAACGA ACGCCGCT | 13395 |
| 8442 | GCGUGCUG A CGACCAGC | 4647 | GCTGGTCG GGCTAGCTACAACGA CAGCACGC | 13396 |
| 8445 | UGCUGACG A CCAGCUGU | 4648 | ACAGCTGG GGCTAGCTACAACGA CGTCAGCA | 13397 |
| 8449 | GACGACCA G CUGUGGUA | 4649 | TACCACAG GGCTAGCTACAACGA TGGTCGTC | 13398 |
| 8452 | GACCAGCU G UGGUAAUA | 4650 | TATTACCA GGCTAGCTACAACGA AGCTGGTC | 13399 |
| 8455 | CAGCUGUG G UAAUACCC | 4651 | GGGTATTA GGCTAGCTACAACGA CACAGCTG | 13400 |
| 8458 | CUGUGGUA A UACCCUCA | 4652 | TGAGGGTA GGCTAGCTACAACGA TACCACAG | 13401 |
| 8460 | GUGGUAAU A CCCUCACA | 4653 | TGTGAGGG GGCTAGCTACAACGA ATTACCAC | 13402 |
| 8466 | AUACCCUC A CAUGUUAC | 4654 | GTAACATG GGCTAGCTACAACGA GAGGGTAT | 13403 |
| 8468 | ACCCUCAC A UGUUACUU | 4655 | AAGTAACA GGCTAGCTACAACGA GTGAGGGT | 13404 |
| 8470 | CCUCACAU G UUACUUGA | 4656 | TCAAGTAA GGCTAGCTACAACGA ATGTGAGG | 13405 |
| 8473 | CACAUGUU A CUUGAAAG | 4657 | CTTTCAAG GGCTAGCTACAACGA AACATGTG | 13406 |
| 8481 | ACUUGAAA G CCUCUGCG | 4658 | CGCAGAGG GGCTAGCTACAACGA TTTCAAGT | 13407 |
| 8487 | AAGCCUCU G CGGCCUGU | 4659 | ACAGGCCG GGCTAGCTACAACGA AGAGGCTT | 13408 |
| 8490 | CCUCUGCG G CCUGUCGA | 4660 | TCGACAGG GGCTAGCTACAACGA CGCAGAGG | 13409 |
| 8494 | UGCGGCCU G UCGAGCUG | 4661 | CAGCTCGA GGCTAGCTACAACGA AGGCCGCA | 13410 |
| 8499 | CCUGUCGA G CUGCGAAG | 4662 | CTTCGCAG GGCTAGCTACAACGA TCGACAGG | 13411 |
| 8502 | GUCGAGCU G CGAAGCUC | 4663 | GAGCTTCG GGCTAGCTACAACGA AGCTCGAC | 13412 |
| 8507 | GCUGCGAA G CUCCAGGA | 4664 | TCCTGGAG GGCTAGCTACAACGA TTCGCAGC | 13413 |
| 8515 | GCUCCAGG A CUGCACGA | 4665 | TCGTGCAG GGCTAGCTACAACGA CCTGGAGC | 13414 |
| 8518 | CCAGGACU G CACGAUGC | 4666 | GCATCGTG GGCTAGCTACAACGA AGTCCTGG | 13415 |
| 8520 | AGGACUGC A CGAUGCUC | 4667 | GAGCATCG GGCTAGCTACAACGA GCAGTCCT | 13416 |
| 8523 | ACUGCACG A UGCUCGUG | 4668 | CACGAGCA GGCTAGCTACAACGA CGTGCAGT | 13417 |
| 8525 | UGCACGAU G CUCCUGUG | 4669 | CACACGAG GGCTAGCTACAACGA ATCGTGCA | 13418 |
| 8529 | CGAUGCUC G UGUGUGGA | 4670 | TCCACACA GGCTAGCTACAACGA GAGCATCG | 13419 |
| 8531 | AUGCUCGU G UGUGGAGA | 4671 | TCTCCACA GGCTAGCTACAACGA ACGAGCAT | 13420 |
| 8533 | GCUCGUGU G UGGAGACG | 4672 | CGTCTCCA GGCTAGCTACAACGA ACACGAGC | 13421 |
| 8539 | GUGUGGAG A CGACCUGG | 4673 | CCAGGTCG GGCTAGCTACAACGA CTCCACAC | 13422 |
| 8542 | UGGAGACG A CCUGGUCG | 4674 | CGACCAGG GGCTAGCTACAACGA CGTCTCCA | 13423 |
| 8547 | ACGACCUG G UCGUUAUC | 4675 | GATAACGA GGCTAGCTACAACGA CAGGTCGT | 13424 |
| 8550 | ACCUGGUC G UUAUCUGU | 4676 | ACAGATAA GGCTAGCTACAACGA GACCAGGT | 13425 |
| 8553 | UGGUCGUU A UCUGUGAA | 4677 | TTCACAGA GGCTAGCTACAACGA AACGACCA | 13426 |
| 8557 | CGUUAUCU G UGAAAGUG | 4678 | CACTTTCA GGCTAGCTACAACGA AGATAACG | 13427 |
| 8563 | CUGUGAAA G UGCGGGGA | 4679 | TCCCCGCA GGCTAGCTACAACGA TTTCACAG | 13428 |
| 8565 | GUGAAAGU G CGGGGACC | 4680 | GGTCCCCG GGCTAGCTACAACGA ACTTTCAC | 13429 |
| 8571 | GUGCGGGG A CCCAAGAG | 4681 | CTCTTGGG GGCTAGCTACAACGA CCCCGCAC | 13430 |
| 8581 | CCAAGAGG A CGCGGCGA | 4682 | TCGCCGCG GGCTAGCTACAACGA CCTCTTGG | 13431 |
| 8583 | AAGAGGAC G CGGCGAGC | 4683 | GCTCGCCG GGCTAGCTACAACGA GTCCTCTT | 13432 |
| 8586 | AGGACGCG G CGAGCCUA | 4684 | TAGGCTCG GGCTAGCTACAACGA CGCGTCCT | 13433 |
| 8590 | CGCGGCGA G CCUACGAG | 4685 | CTCGTAGG GGCTAGCTACAACGA TCGCCGCG | 13434 |
| 8594 | GCGAGCCU A CGAGUCUU | 4686 | AAGACTCG GGCTAGCTACAACGA AGGCTCGC | 13435 |
| 8598 | GCCUACGA G UCUUCACG | 4687 | CGTGAAGA GGCTAGCTACAACGA TCGTAGGC | 13436 |
| 8604 | GAGUCUUC A CGGAGGCU | 4688 | AGCCTCCG GGCTAGCTACAACGA GAAGACTC | 13437 |
| 8610 | UCACGGAG G CUAUGACU | 4689 | AGTCATAG GGCTAGCTACAACGA CTCCGTGA | 13438 |
| 8613 | CGGAGGCU A UGACUAGG | 4690 | CCTAGTCA GGCTAGCTACAACGA AGCCTCCG | 13439 |
| 8616 | AGGCUAUG A CUAGGUAC | 4691 | GTACCTAG GGCTAGCTACAACGA CATAGCCT | 13440 |
| 8621 | AUGACUAG G UACUCUGC | 4692 | GCAGAGTA GGCTAGCTACAACGA CTAGTCAT | 13441 |
| 8623 | GACUAGGU A CUCUGCCC | 4693 | GGGCAGAG GGCTAGCTACAACGA ACCTAGTC | 13442 |
| 8628 | GGUACUCU G CCCCCCCC | 4694 | GGGGGGGG GGCTAGCTACAACGA AGAGTACC | 13443 |
| 8641 | CCCCGGGG A CCCGCCCC | 4695 | GGGGCGGG GGCTAGCTACAACGA CCCCGGGG | 13444 |
| 8645 | GGGGACCC G CCCCAACC | 4696 | GGTTGGGG GGCTAGCTACAACGA GGGTCCCC | 13445 |
| 8651 | CCGCCCCA A CCGGAAUA | 4697 | TATTCCGG GGCTAGCTACAACGA TGGGGCGG | 13446 |
| 8657 | CAACCGGA A UACGACUU | 4698 | AAGTCGTA GGCTAGCTACAACGA TCCGGTTG | 13447 |
| 8659 | ACCGGAAU A CGACUUGG | 4699 | CCAAGTCG GGCTAGCTACAACGA ATTCCGGT | 13448 |
| 8662 | GGAAUACG A CUUGGAGU | 4700 | ACTCCAAG GGCTAGCTACAACGA CGTATTCC | 13449 |
| 8669 | GACUUGGA G UUGAUAAC | 4701 | GTTATCAA GGCTAGCTACAACGA TCCAAGTC | 13450 |
| 8673 | UGGAGUUG A UAACAUCA | 4702 | TGATGTTA GGCTAGCTACAACGA CAACTCCA | 13451 |
| 8676 | AGUUGAUA A CAUCAUGC | 4703 | GCATGATG GGCTAGCTACAACGA TATCAACT | 13452 |
| 8678 | UUGAUAAC A UCAUGCUC | 4704 | GAGCATGA GGCTAGCTACAACGA GTTATCAA | 13453 |
| 8681 | AUAACAUC A UGCUCCUC | 4705 | GAGGAGCA GGCTAGCTACAACGA GATGTTAT | 13454 |
| 8683 | AACAUCAU G CUCCUCCA | 4706 | TGGAGGAG GGCTAGCTACAACGA ATGATGTT | 13455 |
| 8692 | CUCCUCCA A CGUAUCAG | 4707 | CTGATACG GGCTAGCTACAACGA TGGAGGAG | 13456 |
| 8694 | CCUCCAAC G UAUCAGUU | 4708 | AACTGATA GGCTAGCTACAACGA GTTGGAGG | 13457 |
| 8696 | UCCAACGU A UCAGUUGC | 4709 | GCAACTGA GGCTAGCTACAACGA ACGTTGGA | 13458 |
| 8700 | ACGUAUCA G UUGCACAC | 4710 | GTGTGCAA GGCTAGCTACAACGA TGATACGT | 13459 |
| 8703 | UAUCAGUU G CACACGAU | 4711 | ATCGTGTG GGCTAGCTACAACGA AACTGATA | 13460 |
| 8705 | UCAGUUGC A CACGAUGC | 4712 | GCATCGTG GGCTAGCTACAACGA GCAACTGA | 13461 |
| 8707 | AGUUGCAC A CGAUGCAU | 4713 | ATGCATCG GGCTAGCTACAACGA GTGCAACT | 13462 |
| 8710 | UGCACACG A UGCAUCUG | 4714 | CAGATGCA GGCTAGCTACAACGA CGTGTGCA | 13463 |
| 8712 | CACACGAU G CAUCUGGC | 4715 | GCCAGATG GGCTAGCTACAACGA ATCGTGTG | 13464 |
| 8714 | CACGAUGC A UCUGGCAA | 4716 | TTGCCAGA GGCTAGCTACAACGA GCATCGTG | 13465 |
| 8719 | UGCAUCUG G CAAAAGGG | 4717 | CCCTTTTG GGCTAGCTACAACGA CAGATGCA | 13466 |
| 8727 | GCAAAAGG G UGUACUAC | 4718 | GTAGTACA GGCTAGCTACAACGA CCTTTTGC | 13467 |
| 8729 | AAAAGGGU G UACUACCU | 4719 | AGGTAGTA GGCTAGCTACAACGA ACCCTTTT | 13468 |
| 8731 | AAGGGUGU A CUACCUCA | 4720 | TGAGGTAG GGCTAGCTACAACGA ACACCCTT | 13469 |
| 8734 | GGUGUACU A CCUCACCC | 4721 | GGGTGAGG GGCTAGCTACAACGA AGTACACC | 13470 |
| 8739 | ACUACCUC A CCCGUGAC | 4722 | GTCACGGG GGCTAGCTACAACGA GAGGTAGT | 13471 |
| 8743 | CCUCACCC G UGACCCCA | 4723 | TGGGGTCA GGCTAGCTACAACGA GGGTGAGG | 13472 |
| 8746 | CACCCGUG A CCCCACCA | 4724 | TGGTGGGG GGCTAGCTACAACGA CACGGGTG | 13473 |
| 8751 | GUGACCCC A CCACCCCC | 4725 | GGGGGTGG GGCTAGCTACAACGA GGGGTCAC | 13474 |
| 8754 | ACCCCACC A CCCCCCUU | 4726 | AAGGGGGG GGCTAGCTACAACGA GGTGGGGT | 13475 |
| 8763 | CCCCCCUU G CGCGGGCU | 4727 | AGCCCGCG GGCTAGCTACAACGA AAGGGGGG | 13476 |
| 8765 | CCCCUUGC G CGGGCUGC | 4728 | GCAGCCCG GGCTAGCTACAACGA GCAAGGGG | 13477 |
| 8769 | UUGCGCGG G CUGCGUGG | 4729 | CCACGCAG GGCTAGCTACAACGA CCGCGCAA | 13478 |
| 8772 | CGCGGGCU G CGUGGGAG | 4730 | CTCCCACG GGCTAGCTACAACGA AGCCCGCG | 13479 |
| 8774 | CGGGCUGC G UGGUAGAC | 4731 | GTCTCCCA GGCTAGCTACAACGA GCAGCCCG | 13480 |
| 8781 | CGUGGGAG A CAGCUAGA | 4732 | TCTAGCTG GGCTAGCTACAACGA CTCCCACG | 13481 |
| 8784 | GGGAGACA G CUAGAAGC | 4733 | GCTTCTAG GGCTAGCTACAACGA TGTCTCCC | 13482 |
| 8791 | AGCUAGAA G CACUCCAG | 4734 | CTGGAGTG GGCTAGCTACAACGA TTCTAGCT | 13483 |
| 8793 | CUAGAAGC A CUCCAGUC | 4735 | GACTGGAG GGCTAGCTACAACGA GCTTCTAG | 13484 |
| 8799 | GCACUCCA G UCAACUCC | 4736 | GGAGTTGA GGCTAGCTACAACGA TGGAGTGC | 13485 |
| 8803 | UCCAGUCA A CUCCUGGC | 4737 | GCCAGGAG GGCTAGCTACAACGA TGACTGGA | 13486 |
| 8810 | AACUCCUG G CUAGGCAA | 4738 | TTGCCTAG GGCTAGCTACAACGA CAGGAGTT | 13487 |
| 8815 | CUGGCUAG G CAACAUCA | 4739 | TGATGTTG GGCTAGCTACAACGA CTAGCCAG | 13488 |
| 8818 | GCUAGGCA A CAUCAUCA | 4740 | TGATGATG GGCTAGCTACAACGA TGCCTAGC | 13489 |
| 8820 | UAGGCAAC A UCAUCAUG | 4741 | CATGATGA GGCTAGCTACAACGA GTTGCCTA | 13490 |
| 8823 | GCAACAUC A UCAUGUUU | 4742 | AAACATGA GGCTAGCTACAACGA GATGTTGC | 13491 |
| 8826 | ACAUCAUC A UGUUUGCA | 4743 | TGCAAACA GGCTAGCTACAACGA GATGATGT | 13492 |
| 8828 | AUCAUCAU G UUUGCACC | 4744 | GGTGCAAA GGCTAGCTACAACGA ATGATGAT | 13493 |
| 8832 | UCAUGUUU G CACCCACU | 4745 | AGTGGGTG GGCTAGCTACAACGA AAACATGA | 13494 |
| 8834 | AUGUUUGC A CCCACUCU | 4746 | AGAGTGGG GGCTAGCTACAACGA GCAAACAT | 13495 |
| 8838 | UUGCACCC A CUCUAUGG | 4747 | CCATAGAG GGCTAGCTACAACGA GGGTGCAA | 13496 |
| 8843 | CCCACUCU A UGGGUAAG | 4748 | CTTACCCA GGCTAGCTACAACGA AGAGTGGG | 13497 |
| 8847 | CUCUAUGG G UAAGGAUG | 4749 | CATCCTTA GGCTAGCTACAACGA CCATAGAG | 13498 |
| 8853 | GGGUAAGG A UGAUUCUG | 4750 | CAGAATCA GGCTAGCTACAACGA CCTTACCC | 13499 |
| 8856 | UAAGGAUG A UUCUGAUG | 4751 | CATCAGAA GGCTAGCTACAACGA CATCCTTA | 13500 |
| 8862 | UGAUUCUG A UGACUCAC | 4752 | GTGAGTCA GGCTAGCTACAACGA CAGAATCA | 13501 |
| 8865 | UUCUGAUG A CUCACUUC | 4753 | GAAGTGAG GGCTAGCTACAACGA CATCAGAA | 13502 |
| 8869 | GAUGACUC A CUUCUUCU | 4754 | AGAAGAAG GGCTAGCTACAACGA GAGTCATC | 13503 |
| 8880 | UCUUCUCC A UCCUUCUA | 4755 | TAGAAGGA GGCTAGCTACAACGA GGAGAAGA | 13504 |
| 8889 | UCCUUCUA G CCCAGGAG | 4756 | CTCCTGGG GGCTAGCTACAACGA TAGAAGGA | 13505 |
| 8897 | GCCCAGGA G CAACUUGA | 4757 | TCAAGTTG GGCTAGCTACAACGA TCCTGGGC | 13506 |
| 8900 | CAGGAGCA A CUUGAGAA | 4758 | TTCTCAAG GGCTAGCTACAACGA TGCTCCTG | 13507 |
| 8910 | UUGAGAAA G CCCUAGAC | 4759 | GTCTAGGG GGCTAGCTACAACGA TTTCTCAA | 13508 |
| 8917 | AGCCCUAG A CUGCCAGA | 4760 | TCTGGCAG GGCTAGCTACAACGA CTAGGGCT | 13509 |
| 8920 | CCUAGACU G CCAGAUCU | 4761 | AGATCTGG GGCTAGCTACAACGA AGTCTAGG | 13510 |
| 8925 | ACUGCCAG A UCUACGGG | 4762 | CCCGTAGA GGCTAGCTACAACGA CTGGCAGT | 13511 |
| 8929 | CCAGAUCU A CGGGGCUU | 4763 | AAGCCCCG GGCTAGCTACAACGA AGATCTGG | 13512 |
| 8934 | UCUACGGG G CUUGUUAC | 4764 | GTAACAAG GGCTAGCTACAACGA CCCGTAGA | 13513 |
| 8938 | CGGGGCUU G UUACUCCA | 4765 | TGGAGTAA GGCTAGCTACAACGA AAGCCCCG | 13514 |
| 8941 | GGCUUGUU A CUCCAUUG | 4766 | CAATGCAG GGCTAGCTACAACGA AACAAGCC | 13515 |
| 8946 | GUUACUCC A UUGAGCCA | 4767 | TGGCTCAA GGCTAGCTACAACGA GGAGTAAC | 13516 |
| 8951 | UCCAUUGA G CCACUUGA | 4768 | TCAAGTGG GGCTAGCTACAACGA TCAATGGA | 13517 |
| 8954 | AUUGAGCC A CUUGACCU | 4769 | AGGTCAAG GGCTAGCTACAACGA GGCTCAAT | 13518 |
| 8959 | GCCACUUG A CCUACCUC | 4770 | GAGGTAGG GGCTAGCTACAACGA CAAGTGGC | 13519 |
| 8963 | CUUGACCU A CCUCAGAU | 4771 | ATCTGAGG GGCTAGCTACAACGA AGGTCAAG | 13520 |
| 8970 | UACCUCAG A UCAUUCAG | 4772 | CTGAATGA GGCTAGCTACAACGA CTGAGGTA | 13521 |
| 8973 | CUCAGAUC A UUCAGCGA | 4773 | TCGCTGAA GGCTAGCTACAACGA GATCTGAG | 13522 |
| 8978 | AUCAUUCA G CGACUCCA | 4774 | TGGAGTCG GGCTAGCTACAACGA TGAATGAT | 13523 |
| 8981 | AUUCAGCG A CUCCAUGG | 4775 | CCATGGAG GGCTAGCTACAACGA CGCTGAAT | 13524 |
| 8986 | GCGACUCC A UGGUCUUA | 4776 | TAAGACCA GGCTAGCTACAACGA GGAGTCGC | 13525 |
| 8989 | ACUCCAUG G UCUUAGCG | 4777 | CGCTAAGA GGCTAGCTACAACGA CATGGAGT | 13526 |
| 8995 | UGGUCUUA G CGCAUUUU | 4778 | AAAATGCG GGCTAGCTACAACGA TAAGACCA | 13527 |
| 8997 | GUCUUAGC G CAUUUUCA | 4779 | TGAAAATG GGCTAGCTACAACGA GCTAAGAC | 13528 |
| 8999 | CUUAGCGC A UUUUCACU | 4780 | AGTGAAAA GGCTAGCTACAACGA GCGCTAAG | 13529 |
| 9005 | GCAUUUUC A CUCCAUAG | 4781 | CTATGGAG GGCTAGCTACAACGA GAAAATGC | 13530 |
| 9010 | UUCACUCC A UAGUUACU | 4782 | AGTAACTA GGCTAGCTACAACGA GGAGTGAA | 13531 |
| 9013 | ACUCCAUA G UUACUCCC | 4783 | GGGAGTAA GGCTAGCTACAACGA TATGGAGT | 13532 |
| 9016 | CCAUAGUU A CUCCCCAG | 4784 | CTGGGGAG GGCTAGCTACAACGA AACTATGG | 13533 |
| 9025 | CUCCCCAG G UGAAAUCA | 4785 | TGATTTCA GGCTAGCTACAACGA CTGGGGAG | 13534 |
| 9030 | CAGGUGAA A UCAAUAGG | 4786 | CCTATTGA GGCTAGCTACAACGA TTCACCTG | 13535 |
| 9034 | UGAAAUCA A UAGGGUGG | 4787 | CCACCCTA GGCTAGCTACAACGA TGATTTCA | 13536 |
| 9039 | UCAAUAGG G UGGCAUCA | 4788 | TGATGCCA GGCTAGCTACAACGA CCTATTGA | 13537 |
| 9042 | AUAGGGUG G CAUCAUGC | 4789 | GCATGATG GGCTAGCTACAACGA CACCCTAT | 13538 |
| 9044 | AGGGUGGC A UCAUGCCU | 4790 | AGGCATGA GGCTAGCTACAACGA GCCACCCT | 13539 |
| 9047 | GUGGCAUC A UGCCUCAG | 4791 | CTGAGGCA GGCTAGCTACAACGA GATGCCAC | 13540 |
| 9049 | GGCAUCAU G CCUCAGGA | 4792 | TCCTGAGG GGCTAGCTACAACGA ATGATGCC | 13541 |
| 9059 | CUCAGGAA A CUUGGGGU | 4793 | ACCCCAAG GGCTAGCTACAACGA TTCCTGAG | 13542 |
| 9066 | AACUUGGG G UACCACCC | 4794 | GGGTGGTA GGCTAGCTACAACGA CCCAAGTT | 13543 |
| 9068 | CUUGGGGU A CCACCCUU | 4795 | AAGGGTGG GGCTAGCTACAACGA ACCCCAAG | 13544 |
| 9071 | GGGGUACC A CCCUUGCG | 4796 | CGCAAGGG GGCTAGCTACAACGA GGTACCCC | 13545 |
| 9077 | CCACCCUU G CGAACCUG | 4797 | CAGGTTCG GGCTAGCTACAACGA AAGGGTGG | 13546 |
| 9081 | CCUUGCGA A CCUGGAGA | 4798 | TCTCCAGG GGCTAGCTACAACGA TCGCAAGG | 13547 |
| 9089 | ACCUGGAG A CAUCGGGC | 4799 | GCCCGATG GGCTAGCTACAACGA CTCCAGGT | 13548 |
| 9091 | CUGGAGAC A UCGGGCCA | 4800 | TGGCCCGA GGCTAGCTACAACGA GTCTCCAG | 13549 |
| 9096 | GACAUCGG G CCAGAAGU | 4801 | ACTTCTGG GGCTAGCTACAACGA CCGATGTC | 13550 |
| 9103 | GGCCAGAA G UGUUCGCG | 4802 | CGCGAACA GGCTAGCTACAACGA TTCTGGCC | 13551 |
| 9105 | CCAGAAGU G UUCGCGCU | 4803 | AGCGCGAA GGCTAGCTACAACGA ACTTCTGG | 13552 |
| 9109 | AAGUGUUC G CGCUAAGC | 4804 | GCTTAGCG GGCTAGCTACAACGA GAACACTT | 13553 |
| 9111 | GUGUUCGC G CUAAGCUA | 4805 | TAGCTTAG GGCTAGCTACAACGA GCGAACAC | 13554 |
| 9116 | CGCGCUAA G CUACUGUC | 4806 | GACAGTAG GGCTAGCTACAACGA TTAGCGCG | 13555 |
| 9119 | GCUAAGCU A CUGUCCCA | 4807 | TGGGACAG GGCTAGCTACAACGA AGCTTAGC | 13556 |
| 9122 | AAGCUACU G UCCCAGGG | 4808 | CCCTGGGA GGCTAGCTACAACGA AGTAGCTT | 13557 |
| 9138 | GGGGGAGG G CCGCCACC | 4809 | GGTGGCGG GGCTAGCTACAACGA CCTCCCCC | 13558 |
| 9141 | GGAGGGCC G CCACCUGU | 4810 | ACAGGTGG GGCTAGCTACAACGA GGCCCTCC | 13559 |
| 9144 | GGGCCGCC A CCUGUGGC | 4811 | GCCACAGG GGCTAGCTACAACGA GGCGGCCC | 13560 |
| 9148 | CGCCACCU G UGGCAGGU | 4812 | ACCTGCCA GGCTAGCTACAACGA AGGTGGCG | 13561 |
| 9151 | CACCUGUG G CAGGUACC | 4813 | GGTACCTG GGCTAGCTACAACGA CACAGGTG | 13562 |
| 9155 | UGUGGCAG G UACCUCUU | 4814 | AAGAGGTA GGCTAGCTACAACGA CTGCCACA | 13563 |
| 9157 | UGGCAGGU A CCUCUUCA | 4815 | TGAAGAGG GGCTAGCTACAACGA ACCTGCCA | 13564 |
| 9166 | CCUCUUCA A CUGGGCAG | 4816 | CTGCCCAG GGCTAGCTACAACGA TGAAGAGG | 13565 |
| 9171 | UCAACUGG G CAGUAAAG | 4817 | CTTTACTG GGCTAGCTACAACGA CCAGTTGA | 13566 |
| 9174 | ACUGGGCA G UAAAGACC | 4818 | GGTCTTTA GGCTAGCTACAACGA TGCCCAGT | 13567 |
| 9180 | CAGUAAAG A CCAAACUC | 4819 | GAGTTTGG GGCTAGCTACAACGA CTTTACTG | 13568 |
| 9185 | AAGACCAA A CUCAAACU | 4820 | AGTTTGAG GGCTAGCTACAACGA TTGGTCTT | 13569 |
| 9191 | AAACUCAA A CUCACUCC | 4821 | GGAGTGAG GGCTAGCTACAACGA TTGAGTTT | 13570 |
| 9195 | UCAAACUC A CUCCAAUC | 4822 | GATTGGAG GGCTAGCTACAACGA GAGTTTGA | 13571 |
| 9201 | UCACUCCA A UCCCAGCU | 4823 | AGCTGGGA GGCTAGCTACAACGA TGGAGTGA | 13572 |
| 9207 | CAAUCCCA G CUGCGUCU | 4824 | AGACGCAG GGCTAGCTACAACGA TGGGATTG | 13573 |
| 9210 | UCCCAGCU G CGUCUCAG | 4825 | CTGAGACG GGCTAGCTACAACGA AGCTGGGA | 13574 |
| 9212 | CCAGCUGC G UCUCAGUU | 4826 | AACTGAGA GGCTAGCTACAACGA GCAGCTGG | 13575 |
| 9218 | GCGUCUCA G UUGGACUU | 4827 | AAGTCCAA GGCTAGCTACAACGA TGAGACGC | 13576 |
| 9223 | UCAGUUGG A CUUGUCCA | 4828 | TGGACAAG GGCTAGCTACAACGA CCAACTGA | 13577 |
| 9227 | UUGGACUU G UCCAACUG | 4829 | CAGTTGGA GGCTAGCTACAACGA AAGTCCAA | 13578 |
| 9232 | CUUGUCCA A CUGGUUCG | 4830 | CGAACCAG GGCTAGCTACAACGA TGGACAAG | 13579 |
| 9236 | UCCAACUG G UUCGUUGC | 4831 | GCAACGAA GGCTAGCTACAACGA CAGTTGGA | 13580 |
| 9240 | ACUGGUUC G UUGCUGGC | 4832 | GCCAGCAA GGCTACCTACAACGA GAACCAGT | 13581 |
| 9243 | GGUUCGUU G CUGGCUAC | 4833 | GTACCCAG GGCTAGCTACAACGA AACGAACC | 13582 |
| 9247 | CGUUGCUG G CUACAGCG | 4834 | CGCTGTAG GGCTAGCTACAACGA CAGCAACG | 13583 |
| 9250 | UGCUGGCU A CAGCGGGG | 4835 | CCCCGCTG GGCTAGCTACAACGA AGCCAGCA | 13584 |
| 9253 | UGGCUACA G CGGGGGAG | 4836 | CTCCCCCG GGCTAGCTACAACGA TGTAGCCA | 13585 |
| 9262 | CGGGGGAG A CGUGUAUC | 4837 | GATACACG GGCTAGCTACAACGA CTCCCCCG | 13586 |
| 9264 | GGGGAGAC G UGUAUCAC | 4838 | GTGATACA GGCTAGCTACAACGA GTCTCCCC | 13587 |
| 9266 | GGAGACGU G UAUCACAG | 4839 | CTGTGATA GGCTAGCTACAACGA ACGTCTCC | 13588 |
| 9268 | AGACGUGU A UCACAGCC | 4840 | GGCTGTGA GGCTAGCTACAACGA ACACGTCT | 13589 |
| 9271 | CGUGUAUC A CAGCCUGU | 4841 | ACAGGCTG GGCTAGCTACAACGA GATACACG | 13590 |
| 9274 | GUAUCACA G CCUGUCUC | 4842 | GAGACAGG GGCTAGCTACAACGA TGTGATAC | 13591 |
| 9278 | CACAGCCU G UCUCGUGC | 4843 | GCACGAGA GGCTAGCTACAACGA AGGCTGTG | 13592 |
| 9283 | CCUGUCUC G UGCCCGAC | 4844 | GTCGGGCA GGCTAGCTACAACGA GAGACAGG | 13593 |
| 9285 | UGUCUCGU G CCCGACCC | 4845 | GGGTCGGG GGCTAGCTACAACGA ACGAGACA | 13594 |
| 9290 | CGUGCCCG A CCCCGCUG | 4846 | CAGCGGGG GGCTAGCTACAACGA CGGGCACG | 13595 |
| 9295 | CCGACCCC G CUGGUUCA | 4847 | TGAACCAG GGCTAGCTACAACGA GGGGTCGG | 13596 |
| 9299 | CCCCGCUG G UUCAUGCU | 4848 | AGCATGAA GGCTAGCTACAACGA CAGCGGGG | 13597 |
| 9303 | GCUGGUUC A UGCUUUGC | 4849 | GCAAAGCA GGCTAGCTACAACGA GAACCAGC | 13598 |
| 9305 | UGGUUCAU G CUUUGCCU | 4850 | AGGCAAAG GGCTAGCTACAACGA ATGAACCA | 13599 |
| 9310 | CAUGCUUU G CCUACUCC | 4851 | GGAGTAGG GGCTAGCTACAACGA AAAGCATG | 13600 |
| 9314 | CUUUGCCU A CUCCUACU | 4852 | AGTAGGAG GGCTAGCTACAACGA AGGCAAAG | 13601 |
| 9320 | CUACUCCU A CUCUCCGU | 4853 | ACGGAGAG GGCTAGCTACAACGA AGGAGTAG | 13602 |
| 9327 | UACUCUCC G UAGGGGUA | 4854 | TACCCCTA GGCTAGCTACAACGA GGAGAGTA | 13603 |
| 9333 | CCGUAGGG G UAGGCAUC | 4855 | GATGCCTA GGCTAGCTACAACGA CCCTACGG | 13604 |
| 9337 | AGGGGUAG G CAUCUACC | 4856 | GGTAGATG GGCTAGCTACAACGA CTACCCCT | 13605 |
| 9339 | GGGUAGGC A UCUACCUG | 4857 | CAGGTAGA GGCTAGCTACAACGA GCCTACCC | 13606 |
| 9343 | AGGCAUCU A CCUGCUCC | 4858 | GGAGCAGG GGCTAGCTACAACGA AGATGCCT | 13607 |
| 9347 | AUCUACCU G CUCCCCAA | 4859 | TTGGGGAG GGCTAGCTACAACGA AGGTAGAT | 13608 |
| 9355 | GCUCCCCA A CCGAUGAA | 4860 | TTCATCGG GGCTAGCTACAACGA TGGGGAGC | 13609 |
| 9359 | CCCAACCG A UGAACAGG | 4861 | CCTGTTCA GGCTAGCTACAACGA CGGTTGGG | 13610 |
| 9363 | ACCGAUGA A CAGGGAGC | 4862 | GCTCCCTG GGCTAGCTACAACGA TCATCGGT | 13611 |
| 9370 | AACAGGGA G CUAAACAC | 4863 | GTGTTTAG GGCTAGCTACAACGA TCCCTGTT | 13612 |
| 9375 | GGAGCUAA A CACUCCAG | 4864 | CTGGAGTG GGCTAGCTACAACGA TTAGCTCC | 13613 |
| 9377 | AGCUAAAC A CUCCAGGC | 4865 | GCCTGGAG GGCTAGCTACAACGA GTTTAGCT | 13614 |
| 9384 | CACUCCAG G CCAAUAGG | 4866 | CCTATTGG GGCTAGCTACAACGA CTGGAGTG | 13615 |
| 9388 | CCACGCCA A UAGGCCAU | 4867 | ATGGCCTA GGCTAGCTACAACGA TGGCCTGG | 13616 |
| 9392 | GCCAAUAG G CCAUCCCG | 4868 | CGGGATGG GGCTAGCTACAACGA CTATTGGC | 13617 |
| 9395 | AAUAGGCC A UCCCGUUU | 4869 | AAACGGGA GGCTAGCTACAACGA GGCCTATT | 13618 |
| 9400 | GCCAUCCC G UUUUUUUU | 4870 | AAAAAAAA GGCTAGCTACAACGA GGGATGGC | 13619 |

Input Sequence = HPCK1S1.
Cut Site = R/Y
Arm Length = 8.
Core Sequence = GGCTAGCTACAACGA
HPCK1S1 Hepatitis C virus (strain HCV-1b, clone HCV-K1-S1), complete genome; acc# gi|1030702|dbj|D50483.1; 9410 nt

TABLE XIX

HCV minus strand DNAzyme and Substrate Sequence

| | | | | |
|---|---|---|---|---|
| Pos | Substrate | Seq ID | DNAzyme | Seq ID |

| | | | | |
|---|---|---|---|---|
| 9413 | AAAAAAAA A CGGGAUGG | 4871 | CCATCCCG GGCTAGCTACAACGA TTTTTTTT | 13620 |
| 9408 | AAAACGGG A UGGCCUAU | 4872 | ATAGGCCA GGCTAGCTACAACGA CCCGTTTT | 13621 |
| 9405 | ACGGGAUG G CCUAUUGG | 4873 | CCAATAGG GGCTAGCTACAACGA CATCCCGT | 13622 |
| 9401 | GAUGGCCU A UUGGCCUG | 4874 | CAGGCCAA GGCTAGCTACAACGA AGGCCATC | 13623 |
| 9397 | GCCUAUUG G CCUGGAGU | 4875 | ACTCCAGG GGCTAGCTACAACGA CAATAGGC | 13624 |
| 9390 | GGCCUGGA G UGUUUAGC | 4876 | GCTAAACA GGCTAGCTACAACGA TCCAGGCC | 13625 |
| 9388 | CCUGGAGU G UUUAGCUC | 4877 | GAGCTAAA GGCTAGCTACAACGA ACTCCAGG | 13626 |
| 9383 | AGUGUUUA G CUCCCUGU | 4878 | ACAGGGAG GGCTAGCTACAACGA TAAACACT | 13627 |
| 9376 | AGCUCCCU G UUCAUCGG | 4879 | CCGATGAA GGCTAGCTACAACGA AGGGAGCT | 13628 |
| 9372 | CCCUGUUC A UCGGUUGG | 4880 | CCAACCGA GGCTAGCTACAACGA GAACAGGG | 13629 |
| 9368 | GUUCAUCG G UUGGGGAG | 4881 | CTCCCCAA GGCTAGCTACAACGA CGATGAAC | 13630 |
| 9360 | GUUGGGGA G CAGGUAGA | 4882 | TCTACCTG GGCTAGCTACAACGA TCCCCAAC | 13631 |
| 9356 | GGGAGCAG G UAGAUGCC | 4883 | GGCATCTA GGCTAGCTACAACGA CTGCTCCC | 13632 |
| 9352 | GCAGGUAG A UGCCUACC | 4884 | GGTAGGCA GGCTAGCTACAACGA CTACCTGC | 13633 |
| 9350 | AGGUAGAU G CCUACCCC | 4885 | GGGGTAGG GGCTAGCTACAACGA ATCTACCT | 13634 |
| 9346 | AGAUGCCU A CCCCUACG | 4886 | CGTAGGGG GGCTAGCTACAACGA AGGCATCT | 13635 |
| 9340 | CUACCCCU A CGGAGAGU | 4887 | ACTCTCCG GGCTAGCTACAACGA AGGGGTAG | 13636 |
| 9333 | UACGGAGA G UAGGAGUA | 4888 | TACTCCTA GGCTAGCTACAACGA TCTCCGTA | 13637 |
| 9327 | GAGUAGGA G UAGGCAAA | 4889 | TTTGCCTA GGCTAGCTACAACGA TCCTACTC | 13638 |
| 9323 | AGGAGUAG G CAAAGCAU | 4890 | ATGCTTTG GGCTAGCTACAACGA CTACTCCT | 13639 |
| 9318 | UAGGCAAA G CAUGAACC | 4891 | GGTTCATG GGCTAGCTACAACGA TTTGCCTA | 13640 |
| 9316 | GGCAAAGC A UGAACCAG | 4892 | CTGGTTCA GGCTAGCTACAACGA GCTTTGCC | 13641 |
| 9312 | AAGCAUGA A CCAGCGGG | 4893 | CCCGCTGG GGCTAGCTACAACGA TCATGCTT | 13642 |
| 9308 | AUGAACCA G CGGGGUCG | 4894 | CGACCCCG GGCTAGCTACAACGA TGGTTCAT | 13643 |
| 9303 | CCAGCGGG G UCGGGCAC | 4895 | GTGCCCGA GGCTAGCTACAACGA CCCGCTGG | 13644 |
| 9298 | GGGGUCGG G CACGAGAC | 4896 | GTCTCGTG GGCTAGCTACAACGA CCGACCCC | 13645 |
| 9296 | GGUCGGGC A CGAGACAG | 4897 | CTGTCTCG GGCTAGCTACAACGA GCCCGACC | 13646 |
| 9291 | GGCACGAG A CAGGCUGU | 4898 | ACAGCCTG GGCTAGCTACAACGA CTCGTGCC | 13647 |
| 9287 | CGAGACAG G CUGUGAUA | 4899 | TATCACAG GGCTAGCTACAACGA CTGTCTCG | 13648 |
| 9284 | GACAGGCU G UGAUACAC | 4900 | GTGTATCA GGCTAGCTACAACGA AGCCTGTC | 13649 |
| 9281 | AGGCUGUG A UACACGUC | 4901 | GACGTGTA GGCTAGCTACAACGA CACAGCCT | 13650 |
| 9279 | GCUGUGAU A CACGUCUC | 4902 | GAGACGTG GGCTAGCTACAACGA ATCACAGC | 13651 |
| 9277 | UGUGAUAC A CGUCUCCC | 4903 | GGGAGACG GGCTAGCTACAACGA GTATCACA | 13652 |
| 9275 | UGAUACAC G UCUCCCCC | 4904 | GGGGGAGA GGCTAGCTACAACGA GTGTATCA | 13653 |
| 9266 | UCUCCCCC G CUGUAGCC | 4905 | GGCTACAG GGCTAGCTACAACGA GGGGGAGA | 13654 |
| 9263 | CCCCCGCU G UAGCCAGC | 4906 | GCTGGCTA GGCTAGCTACAACGA AGCGGGGG | 13655 |
| 9260 | CCGCUGUA G CCAGCAAC | 4907 | GTTGCTGG GGCTAGCTACAACGA TACAGCGG | 13656 |
| 9256 | UGUAGCCA G CAACGAAC | 4908 | GTTCGTTG GGCTAGCTACAACGA TGGCTACA | 13657 |
| 9253 | AGCCAGCA A CGAACCAG | 4909 | CTGGTTCG GGCTAGCTACAACGA TGCTGGCT | 13658 |
| 9249 | AGCAACGA A CCAGUUGG | 4910 | CCAACTGG GGCTAGCTACAACGA TCGTTGCT | 13659 |
| 9245 | ACGAACCA G UUGGACAA | 4911 | TTGTCCAA GGCTAGCTACAACGA TGGTTCGT | 13660 |
| 9240 | CCAGUUGG A CAAGUCCA | 4912 | TGGACTTG GGCTAGCTACAACGA CCAACTGG | 13661 |
| 9236 | UUGGACAU G UCCAACUG | 4913 | CAGTTGGA GGCTAGCTACAACGA TTGTCCAA | 13662 |
| 9231 | CAAGUCCA A CUGAGACG | 4914 | CGTCTCAG GGCTAGCTACAACGA TGGACTTG | 13663 |
| 9225 | CAACUGAG A CGCAGCUG | 4915 | CAGCTGCG GGCTAGCTACAACGA CTCAGTTG | 13664 |
| 9223 | ACUGAGAC G CAGCUGGG | 4916 | CCCAGCTG GGCTAGCTACAACGA GTCTCAGT | 13665 |
| 9220 | GAGACGCA G CUGGGAUU | 4917 | AATCCCAG GGCTAGCTACAACGA TGCGTCTC | 13666 |
| 9214 | CAGCUGGG A UUGGAGUG | 4918 | CACTCCAA GGCTAGCTACAACGA CCCAGCTG | 13667 |
| 9208 | GGAUUGGA G UGAGUUUG | 4919 | CAAACTCA GGCTAGCTACAACGA TCCAATCC | 13668 |
| 9204 | UGGAGUGA G UUUGAGUU | 4920 | AACTCAAA GGCTAGCTACAACGA TCACTCCA | 13669 |
| 9198 | GAGUUUGA G UUUGGUCU | 4921 | AGACCAAA GGCTAGCTACAACGA TCAAACTC | 13670 |
| 9193 | UGAGUUUG G UCUUUACU | 4922 | AGTAAAGA GGCTAGCTACAACGA CAAACTCA | 13671 |
| 9187 | UGGUCUUU A CUGCCCAG | 4923 | CTGGGCAG GGCTAGCTACAACGA AAAGACCA | 13672 |
| 9184 | UCUUUACU G CCCAGUUG | 4924 | CAACTGGG GGCTAGCTACAACGA AGTAAAGA | 13673 |
| 9179 | ACUGCCCA G UUGAAGAG | 4925 | CTCTTCAA GGCTAGCTACAACGA TGGGCAGT | 13674 |
| 9170 | UUGAAGAG G UACCUGCC | 4926 | GGCAGGTA GGCTAGCTACAACGA CTCTTCAA | 13675 |
| 9168 | GAAGAGGU A CCUGCCAC | 4927 | GTGGCAGG GGCTAGCTACAACGA ACCTCTTC | 13676 |
| 9164 | AGGUACCU G CCACAGGU | 4928 | ACCTGTGG GGCTAGCTACAACGA AGGTACCT | 13677 |
| 9161 | UACCUGCC A CAGGUGGC | 4929 | GCCACCTG GGCTAGCTACAACGA GGCAGGTA | 13678 |
| 9157 | UGCCACAG G UGGCGGCC | 4930 | GGCCGCCA GGCTAGCTACAACGA CTGTGGCA | 13679 |
| 9154 | CACAGGUG G CGGCCCUC | 4931 | GAGGGCCG GGCTAGCTACAACGA CACCTGTG | 13680 |
| 9151 | AGGUGGCG G CCCUCCCC | 4932 | GGGGAGGG GGCTAGCTACAACGA CGCCACCT | 13681 |
| 9135 | CCCCUGGG A CAGUAGCU | 4933 | AGCTACTG GGCTAGCTACAACGA CCCAGGGG | 13682 |
| 9132 | CUGGGACA G UAGCUUAG | 4934 | CTAAGCTA GGCTAGCTACAACGA TGTCCCAG | 13683 |
| 9129 | GGACAGUA G CUUAGCGC | 4935 | GCGCTAAG GGCTAGCTACAACGA TACTGTCC | 13684 |
| 9124 | GUAGCUUA G CGCGAACA | 4936 | TGTTCGCG GGCTAGCTACAACGA TAAGCTAC | 13685 |
| 9122 | AGCUUAGC G CGAACACU | 4937 | AGTGTTCG GGCTAGCTACAACGA GCTAAGCT | 13686 |
| 9118 | UAGCGCGA A CACUUCUG | 4938 | CAGAAGTG GGCTAGCTACAACGA TCGCGCTA | 13687 |
| 9116 | GCGCGAAC A CUUCUGGC | 4939 | GCCAGAAG GGCTAGCTACAACGA GTTCGCGC | 13688 |
| 9109 | CACUUCUG G CCCGAUGU | 4940 | ACATCGGG GGCTAGCTACAACGA CAGAAGTG | 13689 |
| 9104 | CUGGCCCG A UGUCUCCA | 4941 | TGGAGACA GGCTAGCTACAACGA CGGGCCAG | 13690 |
| 9102 | GGCCCGAU G UCUCCAGG | 4942 | CCTGGAGA GGCTAGCTACAACGA ATCGGGCC | 13691 |
| 9094 | GUCUCCAG G UUCGCAAG | 4943 | CTTGCGAA GGCTAGCTACAACGA CTGGAGAC | 13692 |
| 9090 | CCAGGUUC G CAAGGGUG | 4944 | CACCCTTG GGCTAGCTACAACGA GAACCTGG | 13693 |
| 9084 | UCGCAAGG G UGGUACCC | 4945 | GGGTACCA GGCTAGCTACAACGA CCTTGCGA | 13694 |
| 9081 | CAAGGGUG G UACCCCAA | 4946 | TTGGGGTA GGCTAGCTACAACGA CACCCTTG | 13695 |
| 9079 | AGGGUGGU A CCCCAAGU | 4947 | ACTTGGGG GGCTAGCTACAACGA ACCACCCT | 13696 |
| 9072 | UACCCCAA G UUUCCUGA | 4948 | TCAGGAAA GGCTAGCTACAACGA TTGGGGTA | 13697 |
| 9062 | UUCCUGAG G CAUGAUGC | 4949 | GCATCATG GGCTAGCTACAACGA CTCAGGAA | 13698 |
| 9060 | CCUGAGGC A UGAUGCCA | 4950 | TGGCATCA GGCTAGCTACAACGA GCCTCAGG | 13699 |
| 9057 | GAGGCAUG A UGCCACCC | 4951 | GGGTGGCA GGCTAGCTACAACGA CATGCCTC | 13700 |
| 9055 | GGCAUGAU G CCACCCUA | 4952 | TAGGGTGG GGCTAGCTACAACGA ATCATGCC | 13701 |
| 9052 | AUGAUGCC A CCCUAUUG | 4953 | CAATAGGG GGCTAGCTACAACGA GGCATCAT | 13702 |
| 9047 | GCCACCCU A UUGAUUUC | 4954 | GAAATCAA GGCTAGCTACAACGA AGGGTGGC | 13703 |
| 9043 | CCCUAUUG A UUUCACCU | 4955 | AGGTGAAA GGCTAGCTACAACGA CAATAGGG | 13704 |
| 9038 | UUGAUUUC A CCUGGGGA | 4956 | TCCCCAGG GGCTAGCTACAACGA GAAATCAA | 13705 |
| 9029 | CCUGGGGA G UAACUAUG | 4957 | CATAGTTA GGCTAGCTACAACGA TCCCCAGG | 13706 |
| 9026 | GGGGAGUA A CUAUGGAG | 4958 | CTCCATAG GGCTAGCTACAACGA TACTCCCC | 13707 |
| 9023 | GAGUAACU A UGGAGUGA | 4959 | TCACTCCA GGCTAGCTACAACGA AGTTACTC | 13708 |
| 9018 | ACUAUGGA G UGAAAAUG | 4960 | CATTTTCA GGCTAGCTACAACGA TCCATAGT | 13709 |
| 9012 | GAGUGAAA A UGCGCUAA | 4961 | TTAGCGCA GGCTAGCTACAACGA TTTCACTC | 13710 |
| 9010 | GUGAAAAU G CGCUAAGA | 4962 | TCTTAGCG GGCTAGCTACAACGA ATTTTCAC | 13711 |
| 9008 | GAAAAUGC G CUAAGACC | 4963 | GGTCTTAG GGCTAGCTACAACGA GCATTTTC | 13712 |
| 9002 | GCGCUAAG A CCAUGGAG | 4964 | CTCCATGG GGCTAGCTACAACGA CTTAGCGC | 13713 |
| 8999 | CUAAGACC A UGGAGUCG | 4965 | CGACTCCA GGCTAGCTACAACGA GGTCTTAG | 13714 |
| 8994 | ACCAUGGA G UCGCUGAA | 4966 | TTCAGCGA GGCTAGCTACAACGA TCCATGGT | 13715 |
| 8991 | AUGGAGUC G CUGAAUGA | 4967 | TCATTCAG GGCTAGCTACAACGA GACTCCAT | 13716 |
| 8986 | GUCGCUGA A UGAUCUGA | 4968 | TCAGATCA GGCTAGCTACAACGA TCAGCGAC | 13717 |
| 8983 | GCUGAAUG A UCUGAGGU | 4969 | ACCTCAGA GGCTAGCTACAACGA CATTCAGC | 13718 |
| 8976 | GAUCUGAG G UAGGUCAA | 4970 | TTGACCTA GGCTAGCTACAACGA CTCAGATC | 13719 |
| 8972 | UGAGGUAG G UCAAGUGG | 4971 | CCACTTGA GGCTAGCTACAACGA CTACCTCA | 13720 |
| 8967 | UAGGUCAA G UGGCUCAA | 4972 | TTGAGCCA GGCTAGCTACAACGA TTGACCTA | 13721 |
| 8964 | GUCAAGUG G CUCAAUGG | 4973 | CCATTGAG GGCTAGCTACAACGA CACTTGAC | 13722 |
| 8959 | GUGGCUCA A UGGAGUAA | 4974 | TTACTCCA GGCTAGCTACAACGA TGAGCCAC | 13723 |
| 8954 | UCAAUGGA G UAACAAGC | 4975 | GCTTGTTA GGCTAGCTACAACGA TCCATTGA | 13724 |
| 8951 | AUGGAGUA A CAAGCCCC | 4976 | GGGGCTTG GGCTAGCTACAACGA TACTCCAT | 13725 |
| 8947 | AGUAACAA G CCCCGUAG | 4977 | CTACGGGG GGCTAGCTACAACGA TTGTTACT | 13726 |
| 8942 | CAAGCCCC G UAGAUCUG | 4978 | CAGATCTA GGCTAGCTACAACGA GGGGCTTG | 13727 |
| 8938 | CCCCGUAG A UCUGGCAG | 4979 | CTGCCAGA GGCTAGCTACAACGA CTACGGGG | 13728 |
| 8933 | UAGAUCUG G CAGUCUAG | 4980 | CTAGACTG GGCTAGCTACAACGA CAGATCTA | 13729 |
| 8930 | AUCUGGCA G UCUAGGGC | 4981 | GCCCTAGA GGCTAGCTACAACGA TGCCAGAT | 13730 |
| 8923 | AGUCUAGG G CUUUCUCA | 4982 | TGAGAAAG GGCTAGCTACAACGA CCTAGACT | 13731 |
| 8913 | UUUCUCAA G UUGCUCCU | 4983 | AGGAGCAA GGCTAGCTACAACGA TTGAGAAA | 13732 |
| 8910 | CUCAAGUU G CUCCUGGG | 4984 | CCCAGGAG GGCTAGCTACAACGA AACTTGAG | 13733 |
| 8902 | GCUCCUGG G CUAGAAGG | 4985 | CCTTCTAG GGCTAGCTACAACGA CCAGGAGC | 13734 |
| 8893 | CUAGAAGG A UGGAGAAG | 4986 | CTTCTCCA GGCTAGCTACAACGA CCTTCTAG | 13735 |
| 8882 | GAGAAGAA G UGAGUCAU | 4987 | ATGACTCA GGCTAGCTACAACGA TTCTTCTC | 13736 |
| 8878 | AGAAGUGA G UCAUCAGA | 4988 | TCTGATGA GGCTAGCTACAACGA TCACTTCT | 13737 |
| 8875 | AGUGAGUC A UCAGAAUC | 4989 | GATTCTGA GGCTAGCTACAACGA GACTCACT | 13738 |
| 8869 | UCAUCAGA A UCAUCCUU | 4990 | AAGGATGA GGCTAGCTACAACGA TCTGATGA | 13739 |
| 8866 | UCAGAAUC A UCCUUACC | 4991 | GGTAAGGA GGCTAGCTACAACGA GATTCTGA | 13740 |
| 8860 | UCAUCCUU A CCCAUAGA | 4992 | TCTATGGG GGCTAGCTACAACGA AAGGATGA | 13741 |
| 8856 | CCUUACCC A UAGAGUGG | 4993 | CCACTCTA GGCTAGCTACAACGA GGGTAAGG | 13742 |
| 8851 | CCCAUAGA G UGGGUGCA | 4994 | TGCACCCA GGCTAGCTACAACGA TCTATGGG | 13743 |
| 8847 | UAGAGUGG G UGCAAACA | 4995 | TGTTTGCA GGCTAGCTACAACGA CCACTCTA | 13744 |
| 8845 | GAGUGGGU G CAAACAUG | 4996 | CATGTTTG GGCTAGCTACAACGA ACCCACTC | 13745 |
| 8841 | GGGUGCAA A CAUGAUGA | 4997 | TCATCATG GGCTAGCTACAACGA TTGCACCC | 13746 |
| 8839 | GUGCAAAC A UGAUGAUG | 4998 | CATCATCA GGCTAGCTACAACGA GTTTGCAC | 13747 |
| 8836 | CAAACAUG A UGAUGUUG | 4999 | CAACATCA GGCTAGCTACAACGA CATGTTTG | 13748 |
| 8833 | ACAUGAUG A UGUUGCCU | 5000 | AGGCAACA GGCTAGCTACAACGA CATCATGT | 13749 |
| 8831 | AUGAUGAU G UUGCCUAG | 5001 | CTAGGCAA GGCTAGCTACAACGA ATCATCAT | 13750 |
| 8828 | AUGAUGUU G CCUAGCCA | 5002 | TGGCTAGG GGCTAGCTACAACGA AACATCAT | 13751 |
| 8823 | GUUGCCUA G CCAGGAGU | 5003 | ACTCCTGG GGCTAGCTACAACGA TAGGCAAC | 13752 |
| 8816 | AGCCAGGA G UUGACUGG | 5004 | CCAGTCAA GGCTAGCTACAACGA TCCTGGCT | 13753 |
| 8812 | AGGAGUUG A CUGGAGUG | 5005 | CACTCCAG GGCTAGCTACAACGA CAACTCCT | 13754 |
| 8806 | UGACUGGA G UGCUUCUA | 5006 | TAGAAGCA GGCTAGCTACAACGA TCCAGTCA | 13755 |
| 8804 | ACUGGAGU G CUUCUAGC | 5007 | GCTAGAAG GGCTAGCTACAACGA ACTCCAGT | 13756 |
| 8797 | UGCUUCUA G CUGUCUCC | 5008 | GGAGACAG GGCTAGCTACAACGA TAGAAGCA | 13757 |
| 8794 | UUCUAGCU G UCUCCCAC | 5009 | GTGGGAGA GGCTAGCTACAACGA AGCTAGAA | 13758 |
| 8787 | UGUCUCCC A CGCAGCCC | 5010 | GGGCTGCG GGCTAGCTACAACGA GGGAGACA | 13759 |
| 8785 | UCUCCCAC G CAGCCCGC | 5011 | GCGGGCTG GGCTAGCTACAACGA GTGGGAGA | 13760 |
| 8782 | CCCACGCA G CCCGCGCA | 5012 | TGCGCGGG GGCTAGCTACAACGA TGCGTGGG | 13761 |
| 8778 | CGCAGCCC G CGCAAGGG | 5013 | CCCTTGCG GGCTAGCTACAACGA GGGCTGCG | 13762 |
| 8776 | CAGCCCGC G CAAGGGGG | 5014 | CCCCCTTG GGCTAGCTACAACGA GCGGGCTG | 13763 |
| 8767 | CAAGGGGG G UGGUGGGG | 5015 | CCCCACCA GGCTAGCTACAACGA CCCCCTTG | 13764 |
| 8764 | GGGGGGUG G UGGGGUCA | 5016 | TGACCCCA GGCTAGCTACAACGA CACCCCCC | 13765 |
| 8759 | GUGGUGGG G UCACGGGU | 5017 | ACCCGTGA GGCTAGCTACAACGA CCCACCAC | 13766 |
| 8756 | GUGGGGUC A CGGGUGAG | 5018 | CTCACCCG GGCTAGCTACAACGA GACCCCAC | 13767 |
| 8752 | GGUCACGG G UGAGGUAG | 5019 | CTACCTCA GGCTAGCTACAACGA CCGTGACC | 13768 |
| 8747 | CGGGUGAG G UAGUACAC | 5020 | GTGTACTA GGCTAGCTACAACGA CTCACCCG | 13769 |
| 8744 | GUGAGGUA G UACACCCU | 5021 | AGGGTGTA GGCTAGCTACAACGA TACCTCAC | 13770 |
| 8742 | GAGGUAGU A CACCCUUU | 5022 | AAAGGGTG GGCTAGCTACAACGA ACTACCTC | 13771 |
| 8740 | GGUAGUAC A CCCUUUUG | 5023 | CAAAAGGG GGCTAGCTACAACGA GTACTACC | 13772 |
| 8732 | ACCCUUUU G CCAGAUGC | 5024 | GCATCTGG GGCTAGCTACAACGA AAAAGGGT | 13773 |
| 8727 | UUUGCCAG A UGCAUCGU | 5025 | ACGATGCA GGCTAGCTACAACGA CTGGCAAA | 13774 |
| 8725 | UGCCAGAU G CAUCGUGU | 5026 | ACACGATG GGCTAGCTACAACGA ATCTGGCA | 13775 |
| 8723 | CCAGAUGC A UCGUGUGC | 5027 | GCACACGA GGCTAGCTACAACGA GCATCTGG | 13776 |
| 8720 | GAUGCAUC G UGUGCAAC | 5028 | GTTGCACA GGCTAGCTACAACGA GATGCATC | 13777 |
| 8718 | UGCAUCGU G UGCAACUG | 5029 | CAGTTGCA GGCTAGCTACAACGA ACGATGCA | 13778 |
| 8716 | CAUCGUGU G CAACUGAU | 5030 | ATCAGTTG GGCTAGCTACAACGA ACACGATG | 13779 |
| 8713 | CGUGUGCA A CUGAUACG | 5031 | CGTATCAG GGCTAGCTACAACGA TGCACACG | 13780 |
| 8709 | UGCAACUG A UACGUUGG | 5032 | CCAACGTA GGCTAGCTACAACGA CAGTTGCA | 13781 |
| 8707 | CAACUGAU A CGUUGGAG | 5033 | CTCCAACG GGCTAGCTACAACGA ATCAGTTG | 13782 |
| 8705 | ACUCAUAC G UUGGAGGA | 5034 | TCCTCCAA GGCTAGCTACAACGA GTATCAGT | 13783 |
| 8696 | UUGCAGGA G CAUGAUGU | 5035 | ACATCATG GGCTAGCTACAACGA TCCTCCAA | 13784 |
| 8694 | GGAGGAGC A UGAUGUUA | 5036 | TAACATCA GGCTAGCTACAACGA GCTCCTCC | 13785 |
| 8691 | GGAGCAUG A UGUUAUCA | 5037 | TGATAACA GGCTAGCTACAACGA CATGCTCC | 13786 |
| 8689 | AGCAUGAU G UUAUCAAC | 5038 | GTTGATAA GGCTAGCTACAACGA ATCATGCT | 13787 |
| 8686 | AUGAUGUU A UCAACUCC | 5039 | GGAGTTGA GGCTAGCTACAACGA AACATCAT | 13788 |
| 8682 | UGUUAUCA A CUCCAAGU | 5040 | ACTTGGAG GGCTAGCTACAACGA TGATAACA | 13789 |
| 8675 | AACUCCAA G UCGUAUUC | 5041 | GAATACGA GGCTAGCTACAACGA TTGGAGTT | 13790 |
| 8672 | UCCAAGUC G UAUUCCGG | 5042 | CCGGAATA GGCTAGCTACAACGA CACTTGGA | 13791 |
| 8670 | CAAGUCGU A UUCCGGUU | 5043 | AACCGGAA GGCTAGCTACAACGA ACGACTTG | 13792 |
| 8664 | GUAUUCCG G UUGGGGCG | 5044 | CGCCCCAA GGCTAGCTACAACGA CGGAATAC | 13793 |
| 8658 | CGGUUGGG G CGGGUCCC | 5045 | GGGACCCG GGCTAGCTACAACGA CCCAACCG | 13794 |
| 8654 | UGGGGCGG G UCCCCGGG | 5046 | CCCGGGGA GGCTAGCTACAACGA CCGCCCCA | 13795 |
| 8641 | CGGGGGGG G CAGAGUAC | 5047 | GTACTCTG GGCTAGCTACAACGA CCCCCCCG | 13796 |
| 8636 | GGGGCAGA G UACCUAGU | 5048 | ACTAGGTA GGCTAGCTACAACGA TCTGCCCC | 13797 |
| 8634 | GGCAGAGU A CCUAGUCA | 5049 | TGACTAGG GGCTAGCTACAACGA ACTCTGCC | 13798 |
| 8629 | AGUACCUA G UCAUAGCC | 5050 | GGCTATGA GGCTAGCTACAACGA TAGGTACT | 13799 |
| 8626 | ACCUAGUC A UAGCCUCC | 5051 | GGAGGCTA GGCTAGCTACAACGA GACTAGGT | 13800 |
| 8623 | UAGUCAUA G CCUCCGUG | 5052 | CACGGAGG GGCTAGCTACAACGA TATGACTA | 13801 |
| 8617 | UAGCCUCC G UGAAGACU | 5053 | AGTCTTCA GGCTAGCTACAACGA GGAGGCTA | 13802 |
| 8611 | CCGUGAAG A CUCGUAGG | 5054 | CCTACGAG GGCTAGCTACAACGA CTTCACGG | 13803 |
| 8607 | GAAGACUC G UAGGCUCG | 5055 | CGAGCCTA GGCTAGCTACAACGA GAGTCTTC | 13804 |
| 8603 | ACUCGUAG G CUCGCCGC | 5056 | GCGGCGAG GGCTAGCTACAACGA CTACGAGT | 13805 |
| 8599 | GUAGGCUC G CCGCGUCC | 5057 | GGACGCGG GGCTAGCTACAACGA GAGCCTAC | 13806 |
| 8596 | GGCUCGCC G CGUCCUCU | 5058 | AGAGGACG GGCTAGCTACAACGA GGCGAGCC | 13807 |
| 8594 | CUCGCCGC G UCCUCUUG | 5059 | CAAGAGGA GGCTAGCTACAACGA GCGGCGAG | 13808 |
| 8584 | CCUCUUGG G UCCCCGCA | 5060 | TGCGGGGA GGCTAGCTACAACGA CCAAGAGG | 13809 |
| 8578 | GGGUCCCC G CACUUUCA | 5061 | TGAAAGTG GGCTAGCTACAACGA GGGGACCC | 13810 |
| 8576 | GUCCCCGC A CUUUCACA | 5062 | TGTGAAAG GGCTAGCTACAACGA GCGGGGAC | 13811 |
| 8570 | GCACUUUC A CAGAUAAC | 5063 | GTTATCTG GGCTAGCTACAACGA GAAAGTGC | 13812 |
| 8566 | UUUCACAG A UAACGACC | 5064 | GGTCGTTA GGCTAGCTACAACGA CTGTGAAA | 13813 |
| 8563 | CACAGAUA A CGACCAGG | 5065 | CCTGGTCG GGCTAGCTACAACGA TATCTGTG | 13814 |
| 8560 | AGAUAACG A CCAGGUCG | 5066 | CGACCTGG GGCTAGCTACAACGA CGTTATCT | 13815 |
| 8555 | ACGACCAG G UCGUCUCC | 5067 | GGAGACGA GGCTAGCTACAACGA CTGGTCGT | 13816 |
| 8552 | ACCAGGUC G UCUCCACA | 5068 | TGTGGAGA GGCTAGCTACAACGA GACCTGGT | 13817 |
| 8546 | UCGUCUCC A CACACGAG | 5069 | CTCGTGTG GGCTAGCTACAACGA GGAGACGA | 13818 |
| 8544 | GUCUCCAC A CACGAGCA | 5070 | TGCTCGTG GGCTAGCTACAACGA GTGGAGAC | 13819 |
| 8542 | CUCCACAC A CGAGCAUC | 5071 | GATGCTCG GGCTAGCTACAACGA GTGTGGAG | 13820 |
| 8538 | ACACACGA G CAUCGUGC | 5072 | GCACGATG GGCTAGCTACAACGA TCGTGTGT | 13821 |
| 8536 | ACACGAGC A UCGUGCAG | 5073 | CTGCACGA GGCTAGCTACAACGA GCTCGTGT | 13822 |
| 8533 | CGAGCAUC G UGCAGUCC | 5074 | GGACTGCA GGCTAGCTACAACGA GATGCTCG | 13823 |
| 8531 | AGCAUCGU G CAGUCCUG | 5075 | CAGGACTG GGCTAGCTACAACGA ACGATGCT | 13824 |
| 8528 | AUCGUGCA G UCCUGGAG | 5076 | CTCCAGGA GGCTAGCTACAACGA TGCACGAT | 13825 |
| 8520 | GUCCUGGA G CUUCGCAG | 5077 | CTGCGAAG GGCTAGCTACAACGA TCCAGGAC | 13826 |
| 8515 | GGAGCUUC G CAGCUCGA | 5078 | TCGAGCTG GGCTAGCTACAACGA GAAGCTCC | 13827 |
| 8512 | GCUUCGCA G CUCGACAG | 5079 | CTGTCGAG GGCTAGCTACAACGA TGCGAAGC | 13828 |
| 8507 | GCAGCUCG A CAGGCCGC | 5080 | GCGGCCTG GGCTAGCTACAACGA CGAGCTGC | 13829 |
| 8503 | CUCGACAG G CCGCAGAG | 5081 | CTCTGCGG GGCTAGCTACAACGA CTGTCGAG | 13830 |
| 8500 | GACAGGCC G CAGAGGCU | 5082 | AGCCTCTG GGCTAGCTACAACGA GGCCTGTC | 13831 |
| 8494 | CCGCAGAG G CUUUCAAG | 5083 | CTTGAAAG GGCTAGCTACAACGA CTCTGCGG | 13832 |
| 8486 | GCUUUCAA G UAACAUGU | 5084 | ACATGTTA GGCTAGCTACAACGA TTGAAAGC | 13833 |
| 8483 | UUCAAGUA A CAUGUGAG | 5085 | CTCACATG GGCTAGCTACAACGA TACTTGAA | 13834 |
| 8481 | CAAGUAAC A UGUGAGGG | 5086 | CCCTCACA GGCTAGCTACAACGA GTTACTTG | 13835 |
| 8479 | AGUAACAU G UGAGGGUA | 5087 | TACCCTCA GGCTAGCTACAACGA ATGTTACT | 13836 |
| 8473 | AUGUGAGG G UAUUACCA | 5088 | TGGTAATA GGCTAGCTACAACGA CCTCACAT | 13837 |
| 8471 | GUGAGGGU A UUACCACA | 5089 | TGTGGTAA GGCTAGCTACAACGA ACCCTCAC | 13838 |
| 8468 | AGGGUAUU A CCACAGCU | 5090 | AGCTGTGG GGCTAGCTACAACGA AATACCCT | 13839 |
| 8465 | GUAUUACC A CAGCUGGU | 5091 | ACCAGCTG GGCTAGCTACAACGA GGTAATAC | 13840 |
| 8462 | UUACCACA G CUGGUCGU | 5092 | ACGACCAG GGCTAGCTACAACGA TGTGGTAA | 13841 |
| 8458 | CACAGCUG G UCGUCAGC | 5093 | GCTGACGA GGCTAGCTACAACGA CAGCTGTG | 13842 |
| 8455 | AGCUGGUC G UCAGCACG | 5094 | CGTGCTGA GGCTAGCTACAACGA GACCAGCT | 13843 |
| 8451 | GGUCGUCA G CACGCCGC | 5095 | GCGGCGTG GGCTAGCTACAACGA TGACGACC | 13844 |
| 8449 | UCGUCAGC A CGCCGCUC | 5096 | GAGCGGCG GGCTAGCTACAACGA GCTGACGA | 13845 |
| 8447 | GUCAGCAC G CCGCUCGC | 5097 | GCGAGCCG GGCTAGCTACAACGA GTGCTGAC | 13846 |
| 8444 | AGCACGCC G CUCGCGCG | 5098 | CGCGCGAG GGCTAGCTACAACGA GGCGTGCT | 13847 |
| 8440 | CGCCGCUC G CGCGGCAC | 5099 | GTCCCGCG GGCTAGCTACAACGA GAGCGGCG | 13848 |
| 8438 | CCGCUCGC G CGGCACCG | 5100 | CGGTGCCG GGCTAGCTACAACGA GCGAGCGG | 13849 |
| 8435 | CUCGCCCG G CACCCCCG | 5101 | CGCCGCTG GGCTAGCTACAACGA CGCGCCAG | 13850 |
| 8433 | CGCGCGGC A CCGGCGAU | 5102 | ATCGCCGG GGCTAGCTACAACGA GCCGCGCG | 13851 |
| 8429 | CGGCACCG G CGAUAACC | 5103 | GCTTATCG GGCTAGCTACAACGA CGGTGCCG | 13852 |
| 8426 | CACCGGCG A UAACCGCA | 5104 | TGCGGTTA GGCTAGCTACAACGA CGCCCCTG | 13853 |
| 8423 | CGGCGAUA A CCGCACUU | 5105 | AACTGCCC GGCTAGCTACAACGA TATCGCCG | 13854 |
| 8420 | CGAUAACC G CAGUUCUG | 5106 | CAGAACTG GGCTAGCTACAACGA GGTTATCG | 13855 |
| 8417 | UAACCGCA G UUCUGCCC | 5107 | GGGCACAA GGCTAGCTACAACGA TGCGGTTA | 13856 |
| 8412 | GCACUUCU G CCCUUUUG | 5108 | CAAAACCG GGCTAGCTACAACGA ACAACTGC | 13857 |
| 8402 | CCUUUUGA A UUAGUCAG | 5109 | CTGACTAA GGCTAGCTACAACGA TCAAAAGG | 13858 |
| 8398 | UUGAAUUA G UCAGAGGA | 5110 | TCCTCTGA GGCTAGCTACAACGA TAATTCAA | 13859 |
| 8390 | GUCAGAGG A CCCCCGAU | 5111 | ATCGGGGG GGCTAGCTACAACGA CCTCTGAC | 13860 |
| 8383 | GACCCCCG A UAUAAAGC | 5112 | GCTTTATA GGCTAGCTACAACGA CGGGGGTC | 13861 |
| 8381 | CCCCCGAU A UAAAGCCG | 5113 | CGGCTTTA GGCTAGCTACAACGA ATCGGGGG | 13862 |
| 8376 | GAUAUAAA G CCGCUCUG | 5114 | CAGAGCGG GGCTAGCTACAACGA TTTATATC | 13863 |
| 8373 | AUAAAGCC G CUCUGUGA | 5115 | TCACAGAG GGCTAGCTACAACGA GGCTTTAT | 13864 |
| 8368 | GCCGCUCU G UGAGCGAC | 5116 | GTCGCTCA GGCTAGCTACAACGA AGAGCGGC | 13865 |
| 8364 | CUCUGUGA G CGACCUUA | 5117 | TAAGGTCG GGCTAGCTACAACGA TCACAGAG | 13866 |
| 8361 | UGUGAGCG A CCUUAUGG | 5118 | CCATAAGG GGCTAGCTACAACGA CGCTCACA | 13867 |
| 8356 | GCGACCUU A UGGCCUGU | 5119 | ACAGGCCA GGCTAGCTACAACGA AAGGTCGC | 13868 |
| 8353 | ACCUUAUG G CCUGUCUG | 5120 | CAGACAGG GGCTAGCTACAACGA CATAAGGT | 13869 |
| 8349 | UAUGGCCU G UCUGGCUU | 5121 | AAGCCAGA GGCTAGCTACAACGA AGGCCATA | 13870 |
| 8344 | CCUGUCUG G CUUCGGGG | 5122 | CCCCGAAG GGCTAGCTACAACGA CAGACAGG | 13871 |
| 8335 | CUUCGGGG G CCAAGUCA | 5123 | TGACTTGG GGCTAGCTACAACGA CCCCGAAG | 13872 |
| 8330 | GGGGCCAA G UCACAACA | 5124 | TGTTGTGA GGCTAGCTACAACGA TTGGCCCC | 13873 |
| 8327 | GCCAAGUC A CAACAUUG | 5125 | CAATGTTG GGCTAGCTACAACGA GACTTGGC | 13874 |
| 8324 | AAGUCACA A CAUUGGUA | 5126 | TACCAATG GGCTAGCTACAACGA TGTGACTT | 13875 |
| 8322 | GUCACAAC A UUGGUAAA | 5127 | TTTACCAA GGCTAGCTACAACGA GTTGTGAC | 13876 |
| 8318 | CAACAUUG G UAAAUUGA | 5128 | TCAATTTA GGCTAGCTACAACGA CAATGTTG | 13877 |
| 8314 | AUUGGUAA A UUGACUCC | 5129 | GGAGTCAA GGCTAGCTACAACGA TTACCAAT | 13878 |
| 8310 | GUAAAUUG A CUCCUCGA | 5130 | TCGAGGAG GGCTAGCTACAACGA CAATTTAC | 13879 |
| 8302 | ACUCCUCG A CACGGAUG | 5131 | CATCCGTG GGCTAGCTACAACGA CGAGGAGT | 13880 |
| 8300 | UCCUCGAC A CGGAUGUC | 5132 | GACATCCG GGCTAGCTACAACGA GTCGAGGA | 13881 |
| 8296 | CGACACGG A UGUCACUC | 5133 | GAGTGACA GGCTAGCTACAACGA CCGTGTCG | 13882 |
| 8294 | ACACGGAU G UCACUCUC | 5134 | GAGAGTGA GGCTAGCTACAACGA ATCCGTGT | 13883 |
| 8291 | CGGAUGUC A CUCUCGGU | 5135 | ACCGAGAG GGCTAGCTACAACGA GACATCCG | 13884 |
| 8284 | CACUCUCG G UGACUGUU | 5136 | AACAGTCA GGCTAGCTACAACGA CGAGAGTG | 13885 |
| 8281 | UCUCGGUG A CUGUUGAG | 5137 | CTCAACAG GGCTAGCTACAACGA CACCGAGA | 13886 |
| 8278 | CGGUGACU G UUGAGUCG | 5138 | CGACTCAA GGCTAGCTACAACGA AGTCACCG | 13887 |
| 8273 | ACUGUUGA G UCGAAACA | 5139 | TGTTTCGA GGCTAGCTACAACGA TCAACAGT | 13888 |
| 8267 | GAGUCGAA A CAGCGGGU | 5140 | ACCCGCTG GGCTAGCTACAACGA TTCGACTC | 13889 |
| 8264 | UCGAAACA G CGGGUGUC | 5141 | GACACCCG GGCTAGCTACAACGA TGTTTCGA | 13890 |
| 8260 | AACAGCGG G UGUCAUAU | 5142 | ATATGACA GGCTAGCTACAACGA CCGCTGTT | 13891 |
| 8258 | CAGCGGGU G UCAUAUGC | 5143 | GCATATGA GGCTAGCTACAACGA ACCCGCTG | 13892 |
| 8255 | CGGGUGUC A UAUGCAAA | 5144 | TTTGCATA GGCTAGCTACAACGA GACACCCG | 13893 |
| 8253 | GGUGUCAU A UGCAAAGC | 5145 | GCTTTGCA GGCTAGCTACAACGA ATGACACC | 13894 |
| 8251 | UGUCAUAU G CAAAGCCC | 5146 | GGGCTTTG GGCTAGCTACAACGA ATATGACA | 13895 |
| 8246 | UAUGCAAA G CCCAUAGG | 5147 | CCTATGGG GGCTAGCTACAACGA TTTGCATA | 13896 |
| 8242 | CAAAGCCC A UAGGGCAU | 5148 | ATGCCCTA GGCTAGCTACAACGA GGGCTTTG | 13897 |
| 8237 | CCCAUAGG G CAUUUCUU | 5149 | AAGAAATG GGCTAGCTACAACGA CCTATGGG | 13898 |
| 8235 | CAUAGGGC A UUUCUUUG | 5150 | CAAAGAAA GGCTAGCTACAACGA GCCCTATG | 13899 |
| 8226 | UUUCUUUG A UUUCCAGG | 5151 | CCTGGAAA GGCTAGCTACAACGA CAAAGAAA | 13900 |
| 8218 | AUUUCCAG G CAUUCACC | 5152 | GGTGAATG GGCTAGCTACAACGA CTGGAAAT | 13901 |
| 8216 | UUCCAGGC A UUCACCAG | 5153 | CTGGTGAA GGCTAGCTACAACGA GCCTGGAA | 13902 |
| 8212 | AGGCAUUC A CCAGGAAC | 5154 | GTTCCTGG GGCTAGCTACAACGA GAATGCCT | 13903 |
| 8205 | CACCAGGA A CUCAACCC | 5155 | GGGTTGAG GGCTAGCTACAACGA TCCTGGTG | 13904 |
| 8200 | GGAACUCA A CCCGCUGC | 5156 | GCAGCGGG GGCTAGCTACAACGA TGAGTTCC | 13905 |
| 8196 | CUCAACCC G CUGCCCAG | 5157 | CTGGGCAG GGCTAGCTACAACGA GGGTTGAG | 13906 |
| 8193 | AACCCGCU G CCCAGGAG | 5158 | CTCCTGGG GGCTAGCTACAACGA AGCGGGTT | 13907 |
| 8183 | CCAGGAGA G UACUGGAA | 5159 | TTCCAGTA GGCTAGCTACAACGA TCTCCTGG | 13908 |
| 8181 | AGGAGAGU A CUGGAAUC | 5160 | GATTCCAG GGCTAGCTACAACGA ACTCTCCT | 13909 |
| 8175 | GUACUGGA A UCCGUAUG | 5161 | CATACGGA GGCTAGCTACAACGA TCCAGTAC | 13910 |
| 8171 | UGGAAUCC G UAUGAAGA | 5162 | TCTTCATA GGCTAGCTACAACGA GGATTCCA | 13911 |
| 8169 | GAAUCCGU A UGAAGAGC | 5163 | GCTCTTCA GGCTAGCTACAACGA ACGGATTC | 13912 |
| 8162 | UAUGAAGA G CCCAUCAC | 5164 | GTGATGGG GGCTAGCTACAACGA TCTTCATA | 13913 |
| 8158 | AAGAGCCC A UCACGGCC | 5165 | GGCCGTGA GGCTAGCTACAACGA GGGCTCTT | 13914 |
| 8155 | AGCCCAUC A CGGCCUGA | 5166 | TCAGGCCG GGCTAGCTACAACGA GATGGGCT | 13915 |
| 8152 | CCAUCACG G CCUGAGGA | 5167 | TCCTCAGG GGCTAGCTACAACGA CGTGATGG | 13916 |
| 8140 | GAGGAAGG G UGGAGACC | 5168 | GGTCTCCA GGCTAGCTACAACGA CCTTCCTC | 13917 |
| 8134 | GGGUGGAG A CCACGUCG | 5169 | CGACGTGG GGCTAGCTACAACGA CTCCACCC | 13918 |
| 8131 | UGGAGACC A CGUCGUAA | 5170 | TTACGACG GGCTAGCTACAACGA GGTCTCCA | 13919 |
| 8129 | GAGACCAC G UCGUAAAG | 5171 | CTTTACGA GGCTAGCTACAACGA GTGGTCTC | 13920 |
| 8126 | ACCACGUC G UAAAGGGC | 5172 | GCCCTTTA GGCTAGCTACAACGA GACGTGGT | 13921 |
| 8119 | CGUAAAGG G CCAUUUUC | 5173 | GAAAATGG GGCTAGCTACAACGA CCTTTACG | 13922 |
| 8116 | AAAGGGCC A UUUUCUCG | 5174 | CGAGAAAA GGCTAGCTACAACGA GGCCCTTT | 13923 |
| 8108 | AUUVUCUC G CACACACG | 5175 | CGTGTGTG GGCTAGCTACAACGA GAGAAAAT | 13924 |
| 8106 | UUUCUCGC A CACACGAA | 5176 | TTCGTGTG GGCTAGCTACAACGA GCGAGAAA | 13925 |
| 8104 | UCUCGCAC A CACGAACC | 5177 | GGTTCGTG GGCTAGCTACAACGA GTGCGAGA | 13926 |
| 8102 | UCGCACAC A CGAACCCC | 5178 | GGGGTTCG GGCTAGCTACAACGA GTGTGCGA | 13927 |
| 8098 | ACACACGA A CCCCCAAG | 5179 | CTTGGGGG GGCTAGCTACAACGA TCGTGTGT | 13928 |
| 8090 | ACCCCCAA G UCUGGGAA | 5180 | TTCCCAGA GGCTAGCTACAACGA TTGGGGGT | 13929 |
| 8082 | GUCUGGGA A CACGAUAA | 5181 | TTATCGTG GGCTAGCTACAACGA TCCCAGAC | 13930 |
| 8080 | CUGGGAAC A CGAUAAGG | 5182 | CCTTATCG GGCTAGCTACAACGA GTTCCCAG | 13931 |
| 8077 | GGAACACG A UAAGGCGA | 5183 | TCGCCTTA GGCTAGCTACAACGA CGTGTTCC | 13932 |
| 8072 | ACGAUAAG G CGAGCUGG | 5184 | CCAGCTCG GGCTAGCTACAACGA CTTATCGT | 13933 |
| 8068 | UAAGGCGA G CUGGCUUG | 5185 | CAAGCCAG GGCTAGCTACAACGA TCGCCTTA | 13934 |
| 8064 | GCGAGCUG G CUUGCGGC | 5186 | GCCGCAAG GGCTAGCTACAACGA CAGCTCGC | 13935 |
| 8060 | GCUGGCUU G CGGCCUCC | 5187 | GGAGGCCG GGCTAGCTACAACGA AAGCCAGC | 13936 |
| 8057 | GGCUUGCG G CCUCCUUU | 5188 | AAAGGAGG GGCTAGCTACAACGA CGCAAGCC | 13937 |
| 8043 | UUUCUCUG G UUGGACGC | 5189 | GCGTCCAA GGCTAGCTACAACGA CAGAGAAA | 13938 |
| 8038 | CUGGUUGG A CGCAGAAA | 5190 | TTTCTGCG GGCTAGCTACAACGA CCAACCAG | 13939 |
| 8036 | GGUUGGAC G CAGAAAAC | 5191 | GTTTTCTG GGCTAGCTACAACGA GTCCAACC | 13940 |
| 8029 | CGCAGAAA A CCUCAUUU | 5192 | AAATGAGG GGCTAGCTACAACGA TTTCTGCG | 13941 |
| 8024 | AAAACCUC A UUUUUUGC | 5193 | GCAAAAAA GGCTAGCTACAACGA GAGGTTTT | 13942 |
| 8017 | CAUUUUUU G CCAUGAUG | 5194 | CATCATGG GGCTAGCTACAACGA AAAAAATG | 13943 |
| 8014 | UUUUUGCC A UGAUGGUG | 5195 | CACCATCA GGCTAGCTACAACGA GGCAAAAA | 13944 |
| 8011 | UUGCCAUG A UGGUGGUA | 5196 | TACCACCA GGCTAGCTACAACGA CATGGCAA | 13945 |
| 8008 | CCAUGAUG G UGGUAUCA | 5197 | TGATACCA GGCTAGCTACAACGA CATCATGG | 13946 |
| 8005 | UGAUGGUG G UAUCAAUU | 5198 | AATTGATA GGCTAGCTACAACGA CACCATCA | 13947 |
| 8003 | AUGGUGGU A UCAAUUGG | 5199 | CCAATTGA GGCTAGCTACAACGA ACCACCAT | 13948 |
| 7999 | UGGUAUCA A UUGGUGUC | 5200 | GACACCAA GGCTAGCTACAACGA TGATACCA | 13949 |
| 7995 | AUCAAUUG G UGUCUCAG | 5201 | CTGAGACA GGCTAGCTACAACGA CAATTGAT | 13950 |
| 7993 | CAAUUGGU G UCUCAGUG | 5202 | CACTGAGA GGCTAGCTACAACGA ACCAATTG | 13951 |
| 7987 | GUGUCUCA G UGUCUUCC | 5203 | GGAAGACA GGCTAGCTACAACGA TGAGACAC | 13952 |
| 7985 | GUCUCAGU G UCUUCCAG | 5204 | CTGGAAGA GGCTAGCTACAACGA ACTGAGAC | 13953 |
| 7977 | GUCUUCCA G CAAGUCCU | 5205 | AGGACTTG GGCTAGCTACAACGA TGGAAGAC | 13954 |
| 7973 | UCCAGCAA G UCCUUCCA | 5206 | TGGAAGGA GGCTAGCTACAACGA TTGCTGGA | 13955 |
| 7965 | GUCCUUCC A CACGGAGC | 5207 | GCTCCGTG GGCTAGCTACAACGA GGAAGGAC | 13956 |
| 7963 | CCUUCCAC A CGGAGCGG | 5208 | CCGCTCCG GGCTAGCTACAACGA GTGGAAGG | 13957 |
| 7958 | CACACGGA G CGGAUGUG | 5209 | CACATCCG GGCTAGCTACAACGA TCCGTGTG | 13958 |
| 7954 | CGGAGCGG A UGUGGUUG | 5210 | CAACCACA GGCTAGCTACAACGA CCGCTCCG | 13959 |
| 7952 | GAGCGGAU G UGGUUGAC | 5211 | GTCAACCA GGCTAGCTACAACGA ATCCGCTC | 13960 |
| 7949 | CGGAUGUG G UUGACGGC | 5212 | GCCGTCAA GGCTAGCTACAACGA CACATCCG | 13961 |
| 7945 | UGUGGUUG A CGGCCCCG | 5213 | CGGGGCCG GGCTAGCTACAACGA CAACCACA | 13962 |
| 7942 | GGUUGACG G CCCCGCUG | 5214 | CAGCGGGG GGCTAGCTACAACGA CGTCAACC | 13963 |
| 7937 | ACGGCCCC G CUGGAUAG | 5215 | CTATCCAG GGCTAGCTACAACGA GGGGCCGT | 13964 |
| 7932 | CCCGCUGG A UAGGUUCC | 5216 | GGAACCTA GGCTAGCTACAACGA CCAGCGGG | 13965 |
| 7928 | CUGGAUAG G UUCCGGAC | 5217 | GTCCGGAA GGCTAGCTACAACGA CTATCCAG | 13966 |
| 7921 | GGUUCCGG A CGUCCUUU | 5218 | AAAGGACG GGCTAGCTACAACGA CCGGAACC | 13967 |
| 7919 | UUCCGGAC G UCCUUUGC | 5219 | GCAAAGGA GGCTAGCTACAACGA GTCCGGAA | 13968 |
| 7912 | CGUCCUUU G CCCCAUAA | 5220 | TTATGGGG GGCTAGCTACAACGA AAAGGACG | 13969 |
| 7907 | UUUGCCCC A UAACCAAA | 5221 | TTTGGTTA GGCTAGCTACAACGA GGGGCAAA | 13970 |
| 7904 | GCCCCAUA A CCAAAUUU | 5222 | AAATTTGG GGCTAGCTACAACGA TATGGGGC | 13971 |
| 7899 | AUAACCAA A UUUGGACC | 5223 | GGTCCAAA GGCTAGCTACAACGA TTGGTTAT | 13972 |
| 7893 | AAAUUUGG A CCUGGCCG | 5224 | CGGCCAGG GGCTAGCTACAACGA CCAAATTT | 13973 |
| 7888 | UGGACCUG G CCGAAUGU | 5225 | ACATTCGG GGCTAGCTACAACGA CAGGTCCA | 13974 |
| 7883 | CUGGCCGA A UGUGGGGG | 5226 | CCCCCACA GGCTAGCTACAACGA TCGGCCAG | 13975 |
| 7881 | GGCCGAAU G UGGGGGCG | 5227 | CGCCCCCA GGCTAGCTACAACGA ATTCGGCC | 13976 |
| 7875 | AUGUGGGG G CGUCAGUC | 5228 | GACTGACG GGCTAGCTACAACGA CCCCACAT | 13977 |
| 7873 | GUGGGGGC G UCAGUCUG | 5229 | CAGACTGA GGCTAGCTACAACGA GCCCCCAC | 13978 |
| 7869 | GGGCGUCA G UCUGCAGG | 5230 | CCTGCAGA GGCTAGCTACAACGA TGACGCCC | 13979 |
| 7865 | GUCAGUCU G CAGGCUUC | 5231 | GAAGCCTG GGCTAGCTACAACGA AGACTGAC | 13980 |
| 7861 | GUCUGCAG G CUUCCUCU | 5232 | AGAGGAAG GGCTAGCTACAACGA CTGCAGAC | 13981 |
| 7852 | CUUCCUCU A CGGAUAGA | 5233 | TCTATCCG GGCTAGCTACAACGA AGAGGAAG | 13982 |
| 7848 | CUCUACGG A UAGAAGUU | 5234 | AACTTCTA GGCTAGCTACAACGA CCGTAGAG | 13983 |
| 7842 | GGAUAGAA G UUUAGCCU | 5235 | AGGCTAAA GGCTAGCTACAACGA TTCTATCC | 13984 |
| 7837 | GAAGUUUA G CCUUAACU | 5236 | AGTTAAGG GGCTAGCTACAACGA TAAACTTC | 13985 |
| 7831 | UAGCCUUA A CUGUGGAC | 5237 | GTCCACAG GGCTAGCTACAACGA TAAGGCTA | 13986 |
| 7828 | CCUUAACU G UGGACGCC | 5238 | GGCGTCCA GGCTAGCTACAACGA AGTTAAGG | 13987 |
| 7824 | AACUGUGG A CGCCUUCG | 5239 | CGAAGGCG GGCTAGCTACAACGA CCACAGTT | 13988 |
| 7822 | CUGUGGAC G CCUUCGCC | 5240 | GGCGAAGG GGCTAGCTACAACGA GTCCACAG | 13989 |
| 7816 | ACGCCUUC G CCUUCAUC | 5241 | GATGAAGG GGCTAGCTACAACGA GAAGGCGT | 13990 |
| 7810 | UCGCCUUC A UCUCCUUG | 5242 | CAAGGAGA GGCTAGCTACAACGA GAAGGCGA | 13991 |
| 7800 | CUCCUUGA G CACGUCCC | 5243 | GGGACGTG GGCTAGCTACAACGA TCAAGGAG | 13992 |
| 7798 | CCUUGAGC A CGUCCCGG | 5244 | CCGGGACG GGCTAGCTACAACGA GCTCAAGG | 13993 |
| 7796 | UUGAGCAC G UCCCGGUA | 5245 | TACCGGGA GGCTAGCTACAACGA GTGCTCAA | 13994 |
| 7790 | ACGUCCCG G UAGUGGUC | 5246 | GACCACTA GGCTAGCTACAACGA CGGGACGT | 13995 |
| 7787 | UCCCGGUA G UGGUCGUC | 5247 | GACGACCA GGCTAGCTACAACGA TACCGGGA | 13996 |
| 7784 | CGGUAGUG G UCGUCCAG | 5248 | CTGGACGA GGCTAGCTACAACGA CACTACCG | 13997 |
| 7781 | UAGUGGUC G UCCAGGAC | 5249 | GTCCTGGA GGCTAGCTACAACGA GACCACTA | 13998 |
| 7774 | CGUCCAGG A CUUGCAGU | 5250 | ACTGCAAG GGCTAGCTACAACGA CCTGGACG | 13999 |
| 7770 | CAGGACUU G CAGUCUGU | 5251 | ACAGACTG GGCTAGCTACAACGA AAGTCCTG | 14000 |
| 7767 | GACUUGCA G UCUGUCAA | 5252 | TTGACAGA GGCTAGCTACAACGA TGCAAGTC | 14001 |
| 7763 | UGCAGUCU G UCAAAGGU | 5253 | ACCTTTGA GGCTAGCTACAACGA AGACTGCA | 14002 |
| 7756 | UGUCAAAG G UGACCUUC | 5254 | GAAGGTCA GGCTAGCTACAACGA CTTTGACA | 14003 |
| 7753 | CAAAGGUG A CCUUCUUC | 5255 | GAAGAAGG GGCTAGCTACAACGA CACCTTTG | 14004 |
| 7743 | CUUCUUCU G CCGCUGGC | 5256 | GCCAGCGG GGCTAGCTACAACGA AGAAGAAG | 14005 |
| 7740 | CUUCUGCC G CUGGCUUG | 5257 | CAAGCCAG GGCTAGCTACAACGA GGCAGAAG | 14006 |
| 7736 | UGCCGCUG G CUUGCGCU | 5258 | AGCGCAAG GGCTAGCTACAACGA CAGCGGCA | 14007 |
| 7732 | GCUGGCUU G CGCUGCGA | 5259 | TCGCAGCG GGCTAGCTACAACGA AAGCCAGC | 14008 |
| 7730 | UGGCUUGC G CUGCGAGA | 5260 | TCTCGCAG GGCTAGCTACAACGA GCAAGCCA | 14009 |
| 7727 | CUUGCGCU G CGAGAUGU | 5261 | ACATCTCG GGCTAGCTACAACGA AGCGCAAG | 14010 |
| 7722 | GCUGCGAG A UGUUGUAG | 5262 | CTACAACA GGCTAGCTACAACGA CTCGCAGC | 14011 |
| 7720 | UGCGAGAU G UUGUAGCG | 5263 | CGCTACAA GGCTAGCTACAACGA ATCTCGCA | 14012 |
| 7717 | GAGAUGUU G UAGCGUAG | 5264 | CTACGCTA GGCTAGCTACAACGA AACATCTC | 14013 |
| 7714 | AUGUUGUA G CGUAGACC | 5265 | GGTCTACG GGCTAGCTACAACGA TACAACAT | 14014 |
| 7712 | GUUGUAGC G UAGACCAU | 5266 | ATGGTCTA GGCTAGCTACAACGA GCTACAAC | 14015 |
| 7708 | UAGCGUAG A CCAUGUUG | 5267 | CAACATGG GGCTAGCTACAACGA CTACGCTA | 14016 |
| 7705 | CGUAGACC A UGUUGUGG | 5268 | CCACAACA GGCTAGCTACAACGA GGTCTACG | 14017 |
| 7703 | UAGACCAU G UUGUGGUG | 5269 | CACCACAA GGCTAGCTACAACGA ATGGTCTA | 14018 |
| 7700 | ACCAUGUU G UGGUGACG | 5270 | CGTCACCA GGCTAGCTACAACGA AACATGGT | 14019 |
| 7697 | AUGUUGUG G UGACGCAG | 5271 | CTGCGTCA GGCTAGCTACAACGA CACAACAT | 14020 |
| 7694 | UUGUGGUG A CGCAGCAA | 5272 | TTGCTGCG GGCTAGCTACAACGA CACCACAA | 14021 |
| 7692 | GUGGUGAC G CAGCAAAG | 5273 | CTTTGCTG GGCTAGCTACAACGA GTCACCAC | 14022 |
| 7689 | GUGACGCA G CAAAGAGU | 5274 | ACTCTTTG GGCTAGCTACAACGA TGCGTCAC | 14023 |
| 7682 | AGCAAAGA G UUGCUCAA | 5275 | TTGAGCAA GGCTAGCTACAACGA TCTTTGCT | 14024 |
| 7679 | AAAGAGUU G CUCAACGC | 5276 | GCGTTGAG GGCTAGCTACAACGA AACTCTTT | 14025 |
| 7674 | GUUGCUCA A CGCGUUGA | 5277 | TCAACGCG GGCTAGCTACAACGA TGAGCAAC | 14026 |
| 7672 | UGCUCAAC G CGUUGAUG | 5278 | CATCAACG GGCTAGCTACAACGA GTTGAGCA | 14027 |
| 7670 | CUCAACGC G UUGAUGGG | 5279 | CCCATCAA GGCTAGCTACAACGA GCGTTGAG | 14028 |
| 7666 | ACGCGUUG A UGGGCAAC | 5280 | GTTGCCCA GGCTAGCTACAACGA CAACGCGT | 14029 |
| 7662 | GUUGAUGG G CAACUUGC | 5281 | GCAAGTTG GGCTAGCTACAACGA CCATCAAC | 14030 |
| 7659 | GAUGGGCA A CUUGCUUU | 5282 | AAAGCAAG GGCTAGCTACAACGA TGCCCATC | 14031 |
| 7655 | GGCAACUU G CUUUCCUC | 5283 | GAGGAAAG GGCTAGCTACAACGA AAGTTGCC | 14032 |
| 7645 | UUUCCUCC G CAGCGCAU | 5284 | ATGCGCTG GGCTAGCTACAACGA GGAGGAAA | 14033 |
| 7642 | CCUCCGCA G CGCAUGGC | 5285 | GCCATGCG GGCTAGCTACAACGA TGCGGAGG | 14034 |
| 7640 | UCCGCAGC G CAUGGCGU | 5286 | ACGCCATG GGCTAGCTACAACGA GCTGCGGA | 14035 |
| 7638 | CGCAGCGC A UGGCGUGA | 5287 | TCACGCCA GGCTAGCTACAACGA GCGCTGCG | 14036 |
| 7635 | AGCGCAUG G CGUGAUCA | 5288 | TGATCACG GGCTAGCTACAACGA CATGCGCT | 14037 |
| 7633 | CGCAUGGC G UGAUCAGG | 5289 | CCTGATCA GGCTAGCTACAACGA GCCATGCG | 14038 |
| 7630 | AUGGCGUG A UCAGGGCG | 5290 | CGCCCTGA GGCTAGCTACAACGA CACGCCAT | 14039 |
| 7624 | UGAUCAGG G CGCCCGUC | 5291 | GACGGGCG GGCTAGCTACAACGA CCTGATCA | 14040 |
| 7622 | AUCAGGGC G CCCGUCCA | 5292 | TGGACGGG GGCTAGCTACAACGA GCCCTGAT | 14041 |
| 7618 | GGGCGCCC G UCCAUGUG | 5293 | CACATGGA GGCTAGCTACAACGA GGGCGCCC | 14042 |
| 7614 | GCCCGUCC A UGUGUAGG | 5294 | CCTACACA GGCTAGCTACAACGA GGACGGGC | 14043 |
| 7612 | CCGUCCAU G UGUAGGAC | 5295 | GTCCTACA GGCTAGCTACAACGA ATGGACGG | 14044 |
| 7610 | GUCCAUGU G UAGGACAU | 5296 | ATGTCCTA GGCTAGCTACAACGA ACATGGAC | 14045 |
| 7605 | UGUGUAGG A CAUCGAGC | 5297 | GCTCGATG GGCTAGCTACAACGA CCTACACA | 14046 |
| 7603 | UGUAGGAC A UCGAGCAG | 5298 | CTGCTCGA GGCTAGCTACAACGA GTCCTACA | 14047 |
| 7598 | GACAUCGA G CAGCAGAC | 5299 | GTCTGCTG GGCTAGCTACAACGA TCGATGTC | 14048 |
| 7595 | AUCGAGCA G CAGACGAC | 5300 | GTCGTCTG GGCTAGCTACAACGA TGCTCGAT | 14049 |
| 7591 | AGCAGCAG A CGACAUCC | 5301 | GGATGTCG GGCTAGCTACAACGA CTGCTGCT | 14050 |
| 7588 | AGCAGACG A CAUCCUCG | 5302 | CGAGGATG GGCTAGCTACAACGA CGTCTGCT | 14051 |
| 7586 | CAGACGAC A UCCUCGCC | 5303 | GGCGAGGA GGCTAGCTACAACGA GTCGTCTG | 14052 |
| 7580 | ACAUCCUC G CCAGCCUC | 5304 | GAGGCTGG GGCTAGCTACAACGA GAGGATGT | 14053 |
| 7576 | CCUCGCCA G CCUCUUCG | 5305 | CGAAGAGG GGCTAGCTACAACGA TGGCGAGG | 14054 |
| 7568 | GCCUCUUC G CUCACGGU | 5306 | ACCGTGAG GGCTAGCTACAACGA GAAGAGGC | 14055 |
| 7564 | CUUCGCUC A CGGUAGAC | 5307 | GTCTACCG GGCTAGCTACAACGA GAGCGAAG | 14056 |
| 7561 | CGCUCACG G UAGACCAA | 5308 | TTGGTCTA GGCTAGCTACAACGA CGTGAGCG | 14057 |
| 7557 | CACGGUAG A CCAAGACC | 5309 | GGTCTTGG GGCTAGCTACAACGA CTACCGTG | 14058 |
| 7551 | AGACCAAG A CCCGUCGC | 5310 | GCGACGGG GGCTAGCTACAACGA CTTGGTCT | 14059 |
| 7547 | CAAGACCC G UCGCUGAG | 5311 | CTCAGCGA GGCTAGCTACAACGA GGGTCTTG | 14060 |
| 7544 | GACCCGUC G CUGAGAUC | 5312 | GATCTCAG GGCTAGCTACAACGA GACGGGTC | 14061 |
| 7538 | UCGCUGAG A UCGGGAUC | 5313 | GATCCCGA GGCTAGCTACAACGA CTCAGCGA | 14062 |
| 7532 | AGAUCGGG A UCCCCCGG | 5314 | CCGGGGGA GGCTAGCTACAACGA CCCGATCT | 14063 |
| 7524 | AUCCCCCG G CUCCCCCU | 5315 | AGGGGGAG GGCTAGCTACAACGA CGGGGGAT | 14064 |
| 7506 | AAGGGGGG G CAUAGAGG | 5316 | CCTCTATG GGCTAGCTACAACGA CCCCCCTT | 14065 |
| 7504 | GGGGGGGC A UAGAGGAG | 5317 | CTCCTCTA GGCTAGCTACAACGA GCCCCCCC | 14066 |
| 7496 | AUAGAGGA G UACGACUC | 5318 | GAGTCGTA GGCTAGCTACAACGA TCCTCTAT | 14067 |
| 7494 | AGAGGAGU A CGACUCAA | 5319 | TTGAGTCG GGCTAGCTACAACGA ACTCCTCT | 14068 |
| 7491 | GGAGUACG A CUCAACGU | 5320 | ACGTTGAG GGCTAGCTACAACGA CGTACTCC | 14069 |
| 7486 | ACGACUCA A CGUCGGAU | 5321 | ATCCGACG GGCTAGCTACAACGA TGAGTCGT | 14070 |
| 7484 | GACUCAAC G UCGGAUCC | 5322 | GGATCCGA GGCTAGCTACAACGA GTTGAGTC | 14071 |
| 7479 | AACGUCGG A UCCUGCGU | 5323 | ACGCAGGA GGCTAGCTACAACGA CCGACGTT | 14072 |
| 7474 | CGGAUCCU G CGUCACCG | 5324 | CGGTGACG GGCTAGCTACAACGA AGGATCCG | 14073 |
| 7472 | GAUCCUGC G UCACCGUC | 5325 | GACGGTGA GGCTAGCTACAACGA GCAGGATC | 14074 |
| 7469 | CCUGCGUC A CCGUCAUU | 5326 | AATGACGG GGCTAGCTACAACGA GACGCAGG | 14075 |
| 7466 | GCGUCACC G UCAUUGGA | 5327 | TCCAATGA GGCTAGCTACAACGA GGTGACGC | 14076 |
| 7463 | UCACCGUC A UUGGAGGU | 5328 | ACCTCCAA GGCTAGCTACAACGA GACGGTGA | 14077 |
| 7456 | CAUUGGAG G UCUGGUCG | 5329 | CGACCAGA GGCTAGCTACAACGA CTCCAATG | 14078 |
| 7451 | GAGGUCUG G UCGGGGGG | 5330 | CCCCCCGA GGCTAGCTACAACGA CAGACCTC | 14079 |
| 7441 | CGGGGGGG G CGGUUGCC | 5331 | GGCAACCG GGCTAGCTACAACGA CCCCCCCG | 14080 |
| 7438 | GGGGGGCG G UUGCCGUA | 5332 | TACGGCAA GGCTAGCTACAACGA CGCCCCCC | 14081 |
| 7435 | GGGCGGUU G CCGUACCU | 5333 | AGGTACGG GGCTAGCTACAACGA AACCGCCC | 14082 |
| 7432 | CGGUUGCC G UACCUCUA | 5334 | TAGAGGTA GGCTAGCTACAACGA GGCAACCG | 14083 |
| 7430 | GUUGCCGU A CCUCUAUC | 5335 | GATAGAGG GGCTAGCTACAACGA ACGGCAAC | 14084 |
| 7424 | GUACCUCU A UCAGCGGC | 5336 | GCCGCTGA GGCTAGCTACAACGA AGAGGTAC | 14085 |
| 7420 | CUCUAUCA G CGGCCGAU | 5337 | ATCGGCCG GGCTAGCTACAACGA TGATAGAG | 14086 |
| 7417 | UAUCAGCG G CCGAUGAU | 5338 | ATCATCGG GGCTAGCTACAACGA CGCTGATA | 14087 |
| 7413 | AGCGGCCG A UGAUUCAG | 5339 | CTGAATCA GGCTAGCTACAACGA CGGCCGCT | 14088 |
| 7410 | GGCCGAUG A UUCAGAGC | 5340 | GCTCTGAA GGCTAGCTACAACGA CATCGGCC | 14089 |
| 7403 | GAUUCAGA G CUGCCGAA | 5341 | TTCGGCAG GGCTAGCTACAACGA TCTGAATC | 14090 |
| 7400 | UCAGAGCU G CCGAAGGU | 5342 | ACCTTCGG GGCTAGCTACAACGA AGCTCTGA | 14091 |
| 7393 | UGCCGAAG G UCUUUGUG | 5343 | CACAAAGA GGCTAGCTACAACGA CTTCGGCA | 14092 |
| 7387 | AGGUCUUU G UGGCGAGC | 5344 | GCTCGCCA GGCTAGCTACAACGA AAAGACCT | 14093 |
| 7384 | UCUUUGUG G CGAGCUCC | 5345 | GGAGCTCG GGCTAGCTACAACGA CACAAAGA | 14094 |
| 7380 | UGUGGCGA G CUCCGCCA | 5346 | TGGCGGAG GGCTAGCTACAACGA TCGCCACA | 14095 |
| 7375 | CGAGCUCC G CCAAGGCA | 5347 | TGCCTTGG GGCTAGCTACAACGA GGAGCTCG | 14096 |
| 7369 | CCGCCAAG G CAGAAGAC | 5348 | GTCTTCTG GGCTAGCTACAACGA CTTGGCGG | 14097 |
| 7362 | GGCAGAAG A CACGGUGG | 5349 | CCACCGTG GGCTAGCTACAACGA CTTCTGCC | 14098 |
| 7360 | CAGAAGAC A CGGUGGAC | 5350 | GTCCACCG GGCTAGCTACAACGA GTCTTCTG | 14099 |
| 7357 | AAGACACG G UGGACUCU | 5351 | AGAGTCCA GGCTAGCTACAACGA CGTGTCTT | 14100 |
| 7353 | CACGGUGG A CUCUGUCA | 5352 | TGACAGAG GGCTAGCTACAACGA CCACCGTG | 14101 |
| 7348 | UGGACUCU G UCAGAACA | 5353 | TGTTCTGA GGCTAGCTACAACGA AGAGTCCA | 14102 |
| 7342 | CUGUCAGA A CAACCGUC | 5354 | GACGGTTG GGCTAGCTACAACGA TCTGACAG | 14103 |
| 7339 | UCAGAACA A CCGUCCUC | 5355 | GAGGACGG GGCTAGCTACAACGA TGTTCTGA | 14104 |
| 7336 | GAACAACC G UCCUCUUC | 5356 | GAAGAGGA GGCTAGCTACAACGA GGTTGTTC | 14105 |
| 7323 | CUUCCUCC G UGGAGGUG | 5357 | CACCTCCA GGCTAGCTACAACGA GGAGGAAG | 14106 |
| 7317 | CCGUGGAG G UGGUAUUG | 5358 | CAATACCA GGCTAGCTACAACGA CTCCACGG | 14107 |
| 7314 | UGGAGGUG G UAUUGGAG | 5359 | CTCCAATA GGCTAGCTACAACGA CACCTCCA | 14108 |
| 7312 | GAGGUGGU A UUGGAGGG | 5360 | CCCTCCAA GGCTAGCTACAACGA ACCACCTC | 14109 |
| 7303 | UUGGAGGG G CCUUGGCA | 5361 | TGCCAAGG GGCTAGCTACAACGA CCCTCCAA | 14110 |
| 7297 | GGGCCUUG G CAGGUGGC | 5362 | GCCACCTG GGCTAGCTACAACGA CAAGGCCC | 14111 |
| 7293 | CUUGGCAG G UGGCAAUG | 5363 | CATTGCCA GGCTAGCTACAACGA CTGCCAAG | 14112 |
| 7290 | GGCAGGUG G CAAUGGGC | 5364 | GCCCATTG GGCTAGCTACAACGA CACCTGCC | 14113 |
| 7287 | AGGUGGCA A UGGGCACC | 5365 | GGTGCCCA GGCTAGCTACAACGA TGCCACCT | 14114 |
| 7283 | GGCAAUGG G CACCCGUG | 5366 | CACGGGTG GGCTAGCTACAACGA CCATTGCC | 14115 |
| 7281 | CAAUGGGC A CCCGUGUA | 5367 | TACACGGG GGCTAGCTACAACGA GCCCATTG | 14116 |
| 7277 | GGGCACCC G UGUACCAC | 5368 | GTGGTACA GGCTAGCTACAACGA GGGTGCCC | 14117 |
| 7275 | GCACCCGU G UACCACCG | 5369 | CGGTGGTA GGCTAGCTACAACGA ACGGGTGC | 14118 |
| 7273 | ACCCGUGU A CCACCGGA | 5370 | TCCGGTGG GGCTAGCTACAACGA ACACGGGT | 14119 |
| 7270 | CGUGUACC A CCGGAGGG | 5371 | CCCTCCGG GGCTAGCTACAACGA GGTACACG | 14120 |
| 7261 | CCGGAGGG A CGUAGUCU | 5372 | AGACTACG GGCTAGCTACAACGA CCCTCCGG | 14121 |
| 7259 | GGAGGGAC G UAGUCUGG | 5373 | CCAGACTA GGCTAGCTACAACGA GTCCCTCC | 14122 |
| 7256 | GGGACGUA G UCUGGGUC | 5374 | GACCCAGA GGCTAGCTACAACGA TACGTCCC | 14123 |
| 7250 | UAGUCUGG G UCUUUCCA | 5375 | TGGAAAGA GGCTAGCTACAACGA CCAGACTA | 14124 |
| 7239 | UUUCCAGG G CUCUAGUA | 5376 | TACTAGAG GGCTAGCTACAACGA CCTGGAAA | 14125 |
| 7233 | GGGCUCUA G UAGUGGAG | 5377 | CTCCACTA GGCTAGCTACAACGA TAGAGCCC | 14126 |
| 7230 | CUCUAGUA G UGGAGGGU | 5378 | ACCCTCCA GGCTAGCTACAACGA TACTAGAG | 14127 |
| 7223 | AGUGGAGG G UUGUAAUC | 5379 | GATTACAA GGCTAGCTACAACGA CCTCCACT | 14128 |
| 7220 | GGAGGGUU G UAAUCCGG | 5380 | CCGGATTA GGCTAGCTACAACGA AACCCTCC | 14129 |
| 7217 | GGGUUGUA A UCCGGGCG | 5381 | CGCCCGGA GGCTAGCTACAACGA TACAACCC | 14130 |
| 7211 | UAAUCCGG G CGUGCCCA | 5382 | TGGGCACG GGCTAGCTACAACGA CCGGATTA | 14131 |
| 7209 | AUCCGGGC G UGCCCAUA | 5383 | TATGGGCA GGCTAGCTACAACGA GCCCGGAT | 14132 |
| 7207 | CCGGGCGU G CCCAUAUG | 5384 | CATATGGG GGCTAGCTACAACGA ACGCCCGG | 14133 |
| 7203 | GCGUGCCC A UAUGGGUA | 5385 | TACCCATA GGCTAGCTACAACGA GGGCACGC | 14134 |
| 7201 | GUGCCCAU A UGGGUAAC | 5386 | GTTACCCA GGCTAGCTACAACGA ATGGGCAC | 14135 |
| 7197 | CCAUAUGG G UAACGCUG | 5387 | CAGCGTTA GGCTAGCTACAACGA CCATATGG | 14136 |
| 7194 | UAUGGGUA A CGCUGAAG | 5388 | CTTCAGCG GGCTAGCTACAACGA TACCCATA | 14137 |
| 7192 | UGGGUAAC G CUGAAGGA | 5389 | TCCTTCAG GGCTAGCTACAACGA GTTACCCA | 14138 |
| 7182 | UGAAGGAA A CUUCUUGG | 5390 | CCAAGAAG GGCTAGCTACAACGA TTCCTTCA | 14139 |
| 7173 | CUUCUUGG A UUUCCGCA | 5391 | TGCGGAAA GGCTAGCTACAACGA CCAAGAAG | 14140 |
| 7167 | GGAUUUCC G CAGGAUCU | 5392 | AGATCCTG GGCTAGCTACAACGA GGAAATCC | 14141 |
| 7162 | UCCGCAGG A UCUCCGCC | 5393 | GGCGGAGA GGCTAGCTACAACGA CCTGCGGA | 14142 |
| 7156 | GGAUCUCC G CCGGAAUG | 5394 | CATTCCGG GGCTAGCTACAACGA GGAGATCC | 14143 |
| 7150 | CCGCCGGA A UGGACACC | 5395 | GGTGTCCA GGCTAGCTACAACGA TCCGGCGG | 14144 |
| 7146 | CGGAAUGG A CACCUCUC | 5396 | GAGAGGTG GGCTAGCTACAACGA CCATTCCG | 14145 |
| 7144 | GAAUGGAC A CCUCUCUC | 5397 | GAGAGAGG GGCTAGCTACAACGA GTCCATTC | 14146 |
| 7133 | UCUCUCUC A UCCUCCUC | 5398 | GAGGAGGA GGCTAGCTACAACGA GAGAGAGA | 14147 |
| 7123 | CCUCCUCC G CUCGAAGC | 5399 | GCTTCGAG GGCTAGCTACAACGA GGAGGAGG | 14148 |
| 7116 | CGCUCGAA G CGGGUCAA | 5400 | TTGACCCG GGCTAGCTACAACGA TTCGAGCG | 14149 |
| 7112 | CGAAGCGG G UCAAAAGA | 5401 | TCTTTTGA GGCTAGCTACAACGA CCGCTTCG | 14150 |
| 7103 | UCAAAAGA G UCCAGGGU | 5402 | ACCCTGGA GGCTAGCTACAACGA TCTTTTGA | 14151 |
| 7096 | AGUCCAGG G UAACUACC | 5403 | GGTAGTTA GGCTAGCTACAACGA CCTGGACT | 14152 |
| 7093 | CCAGGGUA A CUACCUUA | 5404 | TAAGGTAG GGCTAGCTACAACGA TACCCTGG | 14153 |
| 7090 | GGGUAACU A CCUUAUUC | 5405 | GAATAAGG GGCTAGCTACAACGA AGTTACCC | 14154 |
| 7085 | ACUACCUU A UUCUCUGA | 5406 | TCAGAGAA GGCTAGCTACAACGA AAGGTAGT | 14155 |
| 7077 | AUUCUCUG A CUCCACGC | 5407 | GCGTGGAG GGCTAGCTACAACGA CAGAGAAT | 14156 |
| 7072 | CUGACUCC A CGCGAGUG | 5408 | CACTCGCG GGCTAGCTACAACGA GGAGTCAG | 14157 |
| 7070 | GACUCCAC G CGAGUGAU | 5409 | ATCACTCG GGCTAGCTACAACGA GTGGAGTC | 14158 |
| 7066 | CCACGCGA G UGAUGUUA | 5410 | TAACATCA GGCTAGCTACAACGA TCGCGTGG | 14159 |
| 7063 | CGCGAGUG A UGUUACCG | 5411 | CGGTAACA GGCTAGCTACAACGA CACTCGCG | 14160 |
| 7061 | CGAGUGAU G UUACCGCC | 5412 | GGCGGTAA GGCTAGCTACAACGA ATCACTCG | 14161 |
| 7058 | GUGAUGUU A CCGCCCAU | 5413 | ATGGGCGG GGCTAGCTACAACGA AACATCAC | 14162 |
| 7055 | AUGUUACC G CCCAUCUC | 5414 | GAGATGGG GGCTAGCTACAACGA GGTAACAT | 14163 |
| 7051 | UACCGCCC A UCUCCUGC | 5415 | GCAGGAGA GGCTAGCTACAACGA GGGCGGTA | 14164 |
| 7044 | CAUCUCCU G CCGCCACA | 5416 | TGTGGCGG GGCTAGCTACAACGA AGGAGATG | 14165 |
| 7041 | CUCCUGCC G CCACAGGA | 5417 | TCCTGTGG GGCTAGCTACAACGA GGCAGGAG | 14166 |
| 7038 | CUGCCGCC A CAGGAGGU | 5418 | ACCTCCTG GGCTAGCTACAACGA GGCGGCAG | 14167 |
| 7031 | CACAGGAG G UUGGCCUC | 5419 | GAGGCCAA GGCTAGCTACAACGA CTCCTGTG | 14168 |
| 7027 | GGAGGUUG G CCUCGAUG | 5420 | CATCGAGG GGCTAGCTACAACGA CAACCTCC | 14169 |
| 7021 | UGGCCUCG A UGAGGUCA | 5421 | TGACCTCA GGCTAGCTACAACGA CGAGGCCA | 14170 |
| 7016 | UCGAUGAG G UCAAAGUC | 5422 | GACTTTGA GGCTAGCTACAACGA CTCATCGA | 14171 |
| 7010 | AGGUCAAA G UCUGGGGA | 5423 | TCCCCAGA GGCTAGCTACAACGA TTTGACCT | 14172 |
| 7001 | UCUGGGGA G UCAUAUUG | 5424 | CAATATGA GGCTAGCTACAACGA TCCCCAGA | 14173 |
| 6998 | GGGGAGUC A UAUUGGGU | 5425 | ACCCAATA GGCTAGCTACAACGA GACTCCCC | 14174 |
| 6996 | GGAGUCAU A UUGGGUAA | 5426 | TTACCCAA GGCTAGCTACAACGA ATGACTCC | 14175 |
| 6991 | CAUAUUGG G UAAUGUAU | 5427 | ATACATTA GGCTAGCTACAACGA CCAATATG | 14176 |
| 6988 | AUUGGGUA A UGUAUGUC | 5428 | GACATACA GGCTAGCTACAACGA TACCCAAT | 14177 |
| 6986 | UGGGUAAU G UAUGUCGC | 5429 | GCGACATA GGCTAGCTACAACGA ATTACCCA | 14178 |
| 6984 | GGUAAUGU A UGUCGCCU | 5430 | AGGCGACA GGCTAGCTACAACGA ACATTACC | 14179 |
| 6982 | UAAUGUAU G UCGCCUUC | 5431 | GAAGGCGA GGCTAGCTACAACGA ATACATTA | 14180 |
| 6979 | UGUAUGUC G CCUUCGAA | 5432 | TTCGAAGG GGCTAGCTACAACGA GACATACA | 14181 |
| 6966 | CGAAGAAG G CGCAGACA | 5433 | TGTCTGCG GGCTAGCTACAACGA CTTCTTCG | 14182 |
| 6964 | AAGAAGGC G CAGACAGC | 5434 | GCTGTCTG GGCTAGCTACAACGA GCCTTCTT | 14183 |
| 6960 | AGGCGCAG A CAGCUGGC | 5435 | GCCAGCTG GGCTAGCTACAACGA CTGCGCCT | 14184 |
| 6957 | CGCAGACA G CUGGCUAG | 5436 | CTAGCCAG GGCTAGCTACAACGA TGTCTGCG | 14185 |
| 6953 | GACAGCUG G CUAGCUGA | 5437 | TCAGCTAG GGCTAGCTACAACGA CAGCTGTC | 14186 |
| 6949 | GCUGGCUA G CUGAGGAG | 5438 | CTCCTCAG GGCTAGCTACAACGA TAGCCAGC | 14187 |
| 6941 | GCUGAGGA G CUGGCCAA | 5439 | TTGGCCAG GGCTAGCTACAACGA TCCTCAGC | 14188 |
| 6937 | AGGAGCUG G CCAAGGAG | 5440 | CTCCTTGG GGCTAGCTACAACGA CAGCTCCT | 14189 |
| 6921 | GGGGGGAG A CCCCCUGG | 5441 | CCAGGGGG GGCTAGCTACAACGA CTCCCCCC | 14190 |
| 6913 | ACCCCCUG G CCAGCCUA | 5442 | TAGGCTGG GGCTAGCTACAACGA CAGGGGGT | 14191 |
| 6909 | CCUGGCCA G CCUACGCU | 5443 | AGCGTAGG GGCTAGCTACAACGA TGGCCAGG | 14192 |
| 6905 | GCCAGCCU A CGCUUAGC | 5444 | GCTAAGCG GGCTAGCTACAACGA AGGCTGGC | 14193 |
| 6903 | CAGCCUAC G CUUAGCCG | 5445 | CGGCTAAG GGCTAGCTACAACGA GTAGGCTG | 14194 |
| 6898 | UACGCUUA G CCGUCUCU | 5446 | AGAGACGG GGCTAGCTACAACGA TAAGCGTA | 14195 |
| 6895 | GCUUAGCC G UCUCUCCU | 5447 | AGGAGAGA GGCTAGCTACAACGA GGCTAAGC | 14196 |
| 6886 | UCUCUCCU G UAAUGUGG | 5448 | CCACATTA GGCTAGCTACAACGA AGGAGAGA | 14197 |
| 6883 | CUCCUGUA A UGUGGGAG | 5449 | CTCCCACA GGCTAGCTACAACGA TACAGGAG | 14198 |
| 6881 | CCUGUAAU G UGGGAGGG | 5450 | CCCTCCCA GGCTAGCTACAACGA ATTACAGG | 14199 |
| 6872 | UGGGAGGG G UCGGUGAG | 5451 | CTCACCGA GGCTAGCTACAACGA CCCTCCCA | 14200 |
| 6868 | AGGGGUCG G UGAGCAUG | 5452 | CATGCTCA GGCTAGCTACAACGA CGACCCCT | 14201 |
| 6864 | GUCGGUGA G CAUGGACG | 5453 | CGTCCATG GGCTAGCTACAACGA TCACCGAC | 14202 |
| 6862 | CGGUGAGC A UGGACGUG | 5454 | CACGTCCA GGCTAGCTACAACGA GCTCACCG | 14203 |
| 6858 | GAGCAUGG A CGUGAGCA | 5455 | TGCTCACG GGCTAGCTACAACGA CCATGCTC | 14204 |
| 6856 | GCAUGGAC G UGAGCACU | 5456 | AGTGCTCA GGCTAGCTACAACGA GTCCATGC | 14205 |
| 6852 | GGACGUGA G CACUGCUA | 5457 | TAGCAGTG GGCTAGCTACAACGA TCACGTCC | 14206 |
| 6850 | ACGUGAGC A CUGCUACA | 5458 | TGTAGCAG GGCTAGCTACAACGA GCTCACGT | 14207 |
| 6847 | UGAGCACU G CUACAUCC | 5459 | GGATGTAG GGCTAGCTACAACGA AGTGCTCA | 14208 |
| 6844 | GCACUGCU A CAUCCGGU | 5460 | ACCGGATG GGCTAGCTACAACGA AGCAGTGC | 14209 |
| 6842 | ACUGCUAC A UCCGGUUC | 5461 | GAACCGGA GGCTAGCTACAACGA GTAGCAGT | 14210 |
| 6837 | UACAUCCG G UUCGGGCU | 5462 | AGCCCGAA GGCTAGCTACAACGA CGGATGTA | 14211 |
| 6831 | CGGUUCGG G CUCGCAUG | 5463 | CATGCGAG GGCTAGCTACAACGA CCGAACCG | 14212 |
| 6827 | UCGGGCUC G CAUGGGAG | 5464 | CTCCCATG GGCTAGCTACAACGA GAGCCCGA | 14213 |
| 6825 | GGGCUCGC A UGGGAGCU | 5465 | AGCTCCCA GGCTAGCTACAACGA GCGAGCCC | 14214 |
| 6819 | GCAUGGGA G CUGUGACC | 5466 | GGTCACAG GGCTAGCTACAACGA TCCCATGC | 14215 |
| 6816 | UGGGAGCU G UGACCCAA | 5467 | TTGGGTCA GGCTAGCTACAACGA AGCTCCCA | 14216 |
| 6813 | GAGCUGUG A CCCAACCA | 5468 | TGGTTGGG GGCTAGCTACAACGA CACAGCTC | 14217 |
| 6808 | GUGACCCA A CCAGGUAU | 5469 | ATACCTGG GGCTAGCTACAACGA TGGGTCAC | 14218 |
| 6803 | CCAACCAG G UAUUGGUU | 5470 | AACCAATA GGCTAGCTACAACGA CTGGTTGG | 14219 |
| 6801 | AACCAGGU A UUGGUUGA | 5471 | TCAACCAA GGCTAGCTACAACGA ACCTGGTT | 14220 |
| 6797 | AGGUAUUG G UUGAGCCC | 5472 | GGGCTCAA GGCTAGCTACAACGA CAATACCT | 14221 |
| 6792 | UUGGUUGA G CCCGACCU | 5473 | AGGTCGGG GGCTAGCTACAACGA TCAACCAA | 14222 |
| 6787 | UGAGCCCG A CCUGGAAU | 5474 | ATTCCAGG GGCTAGCTACAACGA CGGGCTCA | 14223 |
| 6780 | GACCUGGA A UGUGACCU | 5475 | AGGTCACA GGCTAGCTACAACGA TCCAGGTC | 14224 |
| 6778 | CCUGGAAU G UGACCUCC | 5476 | GGAGGTCA GGCTAGCTACAACGA ATTCCAGG | 14225 |
| 6775 | GGAAUGUG A CCUCCUCC | 5477 | GGAGGAGG GGCTAGCTACAACGA CACATTCC | 14226 |
| 6765 | CUCCUCCC G UAGGAGAG | 5478 | CTCTCCTA GGCTAGCTACAACGA GGGAGGAG | 14227 |
| 6756 | UAGGAGAG G UCCACACG | 5479 | CGTGTGGA GGCTAGCTACAACGA CTCTCCTA | 14228 |
| 6752 | AGAGGUCC A CACGCCGG | 5480 | CCGGCGTG GGCTAGCTACAACGA GGACCTCT | 14229 |
| 6750 | AGGUCCAC A CGCCGGAG | 5481 | CTCCGGCG GGCTAGCTACAACGA GTGGACCT | 14230 |
| 6748 | GUCCACAC G CCGGAGCG | 5482 | CGCTCCGG GGCTAGCTACAACGA GTGTGGAC | 14231 |
| 6742 | ACGCCGGA G CGUUUCUG | 5483 | CAGAAACG GGCTAGCTACAACGA TCCGGCGT | 14232 |
| 6740 | GCCGGAGC G UUUCUGUG | 5484 | CACAGAAA GGCTAGCTACAACGA GCTCCGGC | 14233 |
| 6734 | GCGUUUCU G UGCAGGCG | 5485 | CGCCTGCA GGCTAGCTACAACGA AGAAACGC | 14234 |
| 6732 | GUUUCUGU G CAGGCGUA | 5486 | TACGCCTG GGCTAGCTACAACGA ACAGAAAC | 14235 |
| 6728 | CUGUGCAG G CGUACCCC | 5487 | GGGGTACG GGCTAGCTACAACGA CTGCACAG | 14236 |
| 6726 | GUGCAGGC G UACCCCAU | 5488 | ATGGGGTA GGCTAGCTACAACGA GCCTGCAC | 14237 |
| 6724 | GCAGGCGU A CCCCAUCC | 5489 | GGATGGGG GGCTAGCTACAACGA ACGCCTGC | 14238 |
| 6719 | CGUACCCC A UCCACUUC | 5490 | GAAGTGGA GGCTAGCTACAACGA GGGGTACG | 14239 |
| 6715 | CCCCAUCC A CUUCCGUG | 5491 | CACGGAAG GGCTAGCTACAACGA GGATGGGG | 14240 |
| 6709 | CCACUUCC G UGAAGAAU | 5492 | ATTCTTCA GGCTAGCTACAACGA GGAAGTGG | 14241 |
| 6702 | CGUGAAGA A UUCGGGGG | 5493 | CCCCCGAA GGCTAGCTACAACGA TCTTCACG | 14242 |
| 6693 | UUCGGGGG G CGGAACCU | 5494 | AGGTTCCG GGCTAGCTACAACGA CCCCCGAA | 14243 |
| 6688 | GGGGCGGA A CCUGGCAC | 5495 | GTGCCAGG GGCTAGCTACAACGA TCCGCCCC | 14244 |
| 6683 | GGAACCUG G CACGGGCA | 5496 | TGCCCGTG GGCTAGCTACAACGA CAGGTTCC | 14245 |
| 6681 | AACCUGGC A CGGGCAUU | 5497 | AATGCCCG GGCTAGCTACAACGA GCCAGGTT | 14246 |
| 6677 | UGGCACGG G CAUUUUAC | 5498 | GTAAAATG GGCTAGCTACAACGA CCGTGCCA | 14247 |
| 6675 | GCACGGGC A UUUUACGU | 5499 | ACGTAAAA GGCTAGCTACAACGA GCCCGTGC | 14248 |
| 6670 | GGCAUUUU A CGUUGUCA | 5500 | TGACAACG GGCTAGCTACAACGA AAAATGCC | 14249 |
| 6668 | CAUUUUAC G UUGUCAGU | 5501 | ACTGACAA GGCTAGCTACAACGA GTAAAATG | 14250 |
| 6665 | UUUACGUU G UCAGUGGU | 5502 | ACCACTGA GGCTAGCTACAACGA AACGTAAA | 14251 |
| 6661 | CGUUGUCA G UGGUCAUG | 5503 | CATGACCA GGCTAGCTACAACGA TGACAACG | 14252 |
| 6658 | UGUCAGUG G UCAUGCCC | 5504 | GGGCATGA GGCTAGCTACAACGA CACTGACA | 14253 |
| 6655 | CAGUGGUC A UGCCCGUC | 5505 | GACGGGCA GGCTAGCTACAACGA GACCACTG | 14254 |
| 6653 | GUGGUCAU G CCCGUCAC | 5506 | GTGACGGG GGCTAGCTACAACGA ATGACCAC | 14255 |
| 6649 | UCAUGCCC G UCACGUAG | 5507 | CTACGTGA GGCTAGCTACAACGA GGGCATGA | 14256 |
| 6646 | UGCCCGUC A CGUAGUGG | 5508 | CCACTACG GGCTAGCTACAACGA GACGGGCA | 14257 |
| 6644 | CCCGUCAC G UAGUGGAA | 5509 | TTCCACTA GGCTAGCTACAACGA GTGACGGG | 14258 |
| 6641 | GUCACGUA G UGGAAAUC | 5510 | GATTTCCA GGCTAGCTACAACGA TACGTGAC | 14259 |
| 6635 | UAGUGGAA A UCCCCCAC | 5511 | GTGGGGGA GGCTAGCTACAACGA TTCCACTA | 14260 |
| 6628 | AAUCCCCC A CCCGCGUA | 5512 | TACGCGGG GGCTAGCTACAACGA GGGGGATT | 14261 |
| 6624 | CCCCACCC G CGUAACCU | 5513 | AGGTTACG GGCTAGCTACAACGA GGGTGGGG | 14262 |
| 6622 | CCACCCGC G UAACCUCC | 5514 | GGAGGTTA GGCTAGCTACAACGA GCGGGTGG | 14263 |
| 6619 | CCCGCGUA A CCUCCACG | 5515 | CGTGGAGG GGCTAGCTACAACGA TACGCGGG | 14264 |
| 6613 | UAACCUCC A CGUACUCC | 5516 | GGAGTACG GGCTAGCTACAACGA GGAGGTTA | 14265 |
| 6611 | ACCUCCAC G UACUCCUC | 5517 | GAGGAGTA GGCTAGCTACAACGA GTGGAGGT | 14266 |
| 6609 | CUCCACGU A CUCCUCAG | 5518 | CTGAGGAG GGCTAGCTACAACGA ACGTGGAG | 14267 |
| 6601 | ACUCCUCA G CGGCCACC | 5519 | GGTGGCCG GGCTAGCTACAACGA TGAGGAGT | 14268 |
| 6598 | CCUCAGCG G CCACCCGC | 5520 | GCGGGTGG GGCTAGCTACAACGA CGCTGAGG | 14269 |
| 6595 | CAGCGGCC A CCCGCCAU | 5521 | ATGGCGGG GGCTAGCTACAACGA GGCCGCTG | 14270 |
| 6591 | GGCCACCC G CCAUAGCG | 5522 | CGCTATGG GGCTAGCTACAACGA GGGTGGCC | 14271 |
| 6588 | CACCCGCC A UAGCGCCC | 5523 | GGGCGCTA GGCTAGCTACAACGA GGCGGGTG | 14272 |
| 6585 | CCGCCAUA G CGCCCUAG | 5524 | CTAGGGCG GGCTAGCTACAACGA TATGGCGG | 14273 |
| 6583 | GCCAUAGC G CCCUAGAA | 5525 | TTCTAGGG GGCTAGCTACAACGA GCTATGGC | 14274 |
| 6575 | GCCCUAGA A UAGUUUGG | 5526 | CCAAACTA GGCTAGCTACAACGA TCTAGGGC | 14275 |
| 6572 | CUAGAAUA G UUUGGCGC | 5527 | GCGCCAAA GGCTAGCTACAACGA TATTCTAG | 14276 |
| 6567 | AUAGUUUG G CGCCGGGG | 5528 | CCCCGGCC GGCTAGCTACAACGA CAAACTAT | 14277 |
| 6565 | AGUUUGGC G CCGGGGAG | 5529 | CTCCCCGG GGCTAGCTACAACGA GCCAAACT | 14278 |
| 6555 | CGGGGAGG G UGUGCAGG | 5530 | CCTGCACA GGCTAGCTACAACGA CCTCCCCG | 14279 |
| 6553 | GGGAGGGU G UGCAGGGG | 5531 | CCCCTGCA GGCTAGCTACAACGA ACCCTCCC | 14280 |
| 6551 | GAGGGUGU G CAGGGGCC | 5532 | GGCCCCTG GGCTAGCTACAACGA ACACCCTC | 14281 |
| 6545 | GUGCAGGG G CCCGUGGU | 5533 | ACCACGGG GGCTAGCTACAACGA CCCTGCAC | 14282 |
| 6541 | AGGGGCCC G UGGUGUAU | 5534 | ATACACCA GGCTAGCTACAACGA GGGCCCCT | 14283 |
| 6538 | GGCCCGUG G UGUAUGCG | 5535 | CGCATACA GGCTAGCTACAACGA CACGGGCC | 14284 |
| 6536 | CCCGUGGU G UAUGCGUU | 5536 | AACGCATA GGCTAGCTACAACGA ACCACGGG | 14285 |
| 6534 | CGUGGUGU A UGCGUUGA | 5537 | TCAACGCA GGCTAGCTACAACGA ACACCACG | 14286 |
| 6532 | UGGUGUAU G CGUUGAUG | 5538 | CATCAACG GGCTAGCTACAACGA ATACACCA | 14287 |
| 6530 | GUGUAUGC G UUGAUGGG | 5539 | CCCATCAA GGCTAGCTACAACGA GCATACAC | 14288 |
| 6526 | AUGCGUUG A UGGGGAAU | 5540 | ATTCCCCA GGCTAGCTACAACGA CAACGCAT | 14289 |
| 6519 | GAUGGGGA A UGUUCCAU | 5541 | ATGGAACA GGCTAGCTACAACGA TCCCCATC | 14290 |
| 6517 | UGGGGAAU G UUCCAUGC | 5542 | GCATGGAA GGCTAGCTACAACGA ATTCCCCA | 14291 |
| 6512 | AAUGUUCC A UGCCACGU | 5543 | ACGTGGCA GGCTAGCTACAACGA GGAACATT | 14292 |
| 6510 | UGUUCCAU G CCACGUGU | 5544 | ACACGTGG GGCTAGCTACAACGA ATGGAACA | 14293 |
| 6507 | UCCAUGCC A CGUGUUGC | 5545 | GCAACACG GGCTAGCTACAACGA GGCATGGA | 14294 |
| 6505 | CAUGCCAC G UGUUGCUA | 5546 | TAGCAACA GGCTAGCTACAACGA GTGGCATG | 14295 |
| 6503 | UGCCACGU G UUGCUACA | 5547 | TGTAGCAA GGCTAGCTACAACGA ACGTGGCA | 14296 |
| 6500 | CACGUGUU G CUACAGGU | 5548 | ACCTGTAG GGCTAGCTACAACGA AACACGTG | 14297 |
| 6497 | GUGUUGCU A CAGGUCUU | 5549 | AAGACCTG GGCTAGCTACAACGA AGCAACAC | 14298 |
| 6493 | UGCUACAG G UCUUAGGC | 5550 | GCCTAAGA GGCTAGCTACAACGA CTGTAGCA | 14299 |
| 6486 | GGUCUUAG G CCCGACGA | 5551 | TCGTCGGG GGCTAGCTACAACGA CTAAGACC | 14300 |
| 6481 | UAGGCCCG A CGAUCCUC | 5552 | GAGGATCG GGCTAGCTACAACGA CGGGCCTA | 14301 |
| 6478 | GCCCGACG A UCCUCAUG | 5553 | CATGAGGA GGCTAGCTACAACGA CGTCGGGC | 14302 |
| 6472 | CGAUCCUC A UGGAACCG | 5554 | CGGTTCCA GGCTAGCTACAACGA GAGGATCG | 14303 |
| 6467 | CUCAUGGA A CCGUUCUU | 5555 | AAGAACGG GGCTAGCTACAACGA TCCATGAG | 14304 |
| 6464 | AUGGAACC G UUCUUGAC | 5556 | GTCAAGAA GGCTAGCTACAACGA GGTTCCAT | 14305 |
| 6457 | CGUUCUUG A CAUGUCCA | 5557 | TGGACATG GGCTAGCTACAACGA CAAGAACG | 14306 |
| 6455 | UUCUUGAC A UGUCCAGU | 5558 | ACTGGACA GGCTAGCTACAACGA GTCAAGAA | 14307 |
| 6453 | CUUGACAU G UCCAGUGA | 5559 | TCACTGGA GGCTAGCTACAACGA ATGTCAAG | 14308 |
| 6448 | CAUGUCCA G UGAUCUGC | 5560 | GCAGATCA GGCTAGCTACAACGA TGGACATG | 14309 |
| 6445 | GUCCAGUG A UCUGCGCU | 5561 | AGCGCAGA GGCTAGCTACAACGA CACTGGAC | 14310 |
| 6441 | AGUGAUCU G CGCUCCGC | 5562 | GCGGAGCG GGCTAGCTACAACGA AGATCACT | 14311 |
| 6439 | UGAUCUGC G CUCCGCAU | 5563 | ATGCGGAG GGCTAGCTACAACGA GCAGATCA | 14312 |
| 6434 | UGCGCUCC G CAUGGGCA | 5564 | TGCCCATG GGCTAGCTACAACGA GGAGCGCA | 14313 |
| 6432 | CGCUCCGC A UGGGCAGG | 5565 | CCTGCCCA GGCTAGCTACAACGA GCGGAGCG | 14314 |
| 6428 | CCGCAUGG G CAGGUGGU | 5566 | ACCACCTG GGCTAGCTACAACGA CCATGCGG | 14315 |
| 6424 | AUGGGCAG G UGGUUUGC | 5567 | GCAAACCA GGCTAGCTACAACGA CTGCCCAT | 14316 |
| 6421 | GGCAGGUG G UUUGCAUG | 5568 | CATGCAAA GGCTAGCTACAACGA CACCTGCC | 14317 |
| 6417 | GGUGGUUU G CAUGAUAC | 5569 | GTATCATG GGCTAGCTACAACGA AAACCACC | 14318 |
| 6415 | UGGUUUGC A UGAUACCG | 5570 | CGGTATCA GGCTAGCTACAACGA GCAAACCA | 14319 |
| 6412 | UUUGCAUG A UACCGUCU | 5571 | AGACGGTA GGCTAGCTACAACGA CATGCAAA | 14320 |
| 6410 | UGCAUGAU A CCGUCUCC | 5572 | GGAGACGG GGCTAGCTACAACGA ATCATGCA | 14321 |
| 6407 | AUGAUACC G UCUCCCCG | 5573 | CGGGGAGA GGCTAGCTACAACGA GGTATCAT | 14322 |
| 6399 | GUCUCCCC G CCAGACCC | 5574 | GGGTCTGG GGCTAGCTACAACGA GGGGAGAC | 14323 |
| 6394 | CCCGCCAG A CCCCCCUG | 5575 | CAGGGGGG GGCTAGCTACAACGA CTGGCGGG | 14324 |
| 6386 | ACCCCCCU G UACCCACG | 5576 | CGTGGGTA GGCTAGCTACAACGA AGGGGGGT | 14325 |
| 6384 | CCCCCUGU A CCCACGUU | 5577 | AACGTGGG GGCTAGCTACAACGA ACAGGGGG | 14326 |
| 6380 | CUGUACCC A CGUUGGCA | 5578 | TGCCAACG GGCTAGCTACAACGA GGGTACAG | 14327 |
| 6378 | GUACCCAC G UUGGCAUG | 5579 | CATGCCAA GGCTAGCTACAACGA GTGGGTAC | 14328 |
| 6374 | CCACGUUG G CAUGAGAA | 5580 | TTCTCATG GGCTAGCTACAACGA CAACGTGG | 14329 |
| 6372 | ACGUUGGC A UGAGAAGA | 5581 | TCTTCTCA GGCTAGCTACAACGA GCCAACGT | 14330 |
| 6358 | AGAAAGGG A CUCCCGGC | 5582 | GCCGGGAG GGCTAGCTACAACGA CCCTTTCT | 14331 |
| 6351 | GACUCCCG G CAACCGCG | 5583 | CGCGGTTG GGCTAGCTACAACGA CGGGAGTC | 14332 |
| 6348 | UCCCGGCA A CCGCGGCA | 5584 | TGCCGCGG GGCTAGCTACAACGA TGCCGGGA | 14333 |
| 6345 | CGGCAACC G CGGCAGGA | 5585 | TCCTGCCG GGCTAGCTACAACGA GGTTGCCG | 14334 |
| 6342 | CAACCGCG G CAGGAGCU | 5586 | AGCTCCTG GGCTAGCTACAACGA CGCGGTTG | 14335 |
| 6336 | CGGCAGGA G CUUGGACU | 5587 | AGTCCAAG GGCTAGCTACAACGA TCCTGCCG | 14336 |
| 6330 | GAGCUUGG A CUGAAGCC | 5588 | GGCTTCAG GGCTAGCTACAACGA CCAAGCTC | 14337 |
| 6324 | GGACUGAA G CCAGGUCU | 5589 | AGACCTGG GGCTAGCTACAACGA TTCAGTCC | 14338 |
| 6319 | GAAGCCAG G UCUUGAAG | 5590 | CTTCAAGA GGCTAGCTACAACGA CTGGCTTC | 14339 |
| 6311 | GUCUUGAA G UCAGUCAA | 5591 | TTGACTGA GGCTAGCTACAACGA TTCAAGAC | 14340 |
| 6307 | UGAAGUCA G UCAACACC | 5592 | GGTGTTGA GGCTAGCTACAACGA TGACTTCA | 14341 |
| 6303 | GUCAGUCA A CACCGUGC | 5593 | GCACGGTG GGCTAGCTACAACGA TGACTGAC | 14342 |
| 6301 | CAGUCAAC A CCGUGCAU | 5594 | ATGCACGG GGCTAGCTACAACGA GTTGACTG | 14343 |
| 6298 | UCAACACC G UGCAUAUC | 5595 | GATATGCA GGCTAGCTACAACGA GGTGTTGA | 14344 |
| 6296 | AACACCGU G CAUAUCCA | 5596 | TGGATATG GGCTAGCTACAACGA ACGGTGTT | 14345 |
| 6294 | CACCGUGC A UAUCCAGU | 5597 | ACTGGATA GGCTAGCTACAACGA GCACGGTG | 14346 |
| 6292 | CCGUGCAU A UCCAGUCC | 5598 | GGACTGGA GGCTAGCTACAACGA ATGCACGG | 14347 |
| 6287 | CAUAUCCA G UCCCAAAC | 5599 | GTTTGGGA GGCTAGCTACAACGA TGGATATG | 14348 |
| 6280 | AGUCCCAA A CAUCCCUU | 5600 | AAGGGATG GGCTAGCTACAACGA TTGGGACT | 14349 |
| 6278 | UCCCAAAC A UCCCUUAG | 5601 | CTAAGGGA GGCTAGCTACAACGA GTTTGGGA | 14350 |
| 6270 | AUCCCUUA G CCACGAGC | 5602 | GCTCGTGG GGCTAGCTACAACGA TAAGGGAT | 14351 |
| 6267 | CCUUAGCC A CGAGCCGG | 5603 | CCGGCTCG GGCTAGCTACAACGA GGCTAAGG | 14352 |
| 6263 | AGCCACGA G CCGGAACA | 5604 | TGTTCCGG GGCTAGCTACAACGA TCGTGGCT | 14353 |
| 6257 | GAGCCGGA A CAUGGCGU | 5605 | ACGCCATG GGCTAGCTACAACGA TCCGGCTC | 14354 |
| 6255 | GCCGGAAC A UGGCGUGG | 5606 | CCACGCCA GGCTAGCTACAACGA GTTCCGGC | 14355 |
| 6252 | GGAACAUG G CGUGGAGC | 5607 | GCTCCACG GGCTAGCTACAACGA CATGTTCC | 14356 |
| 6250 | AACAUGGC G UGGAGCAG | 5608 | CTGCTCCA GGCTAGCTACAACGA GCCATGTT | 14357 |
| 6245 | GGCGUGGA G CAGUCCUC | 5609 | GAGGACTG GGCTAGCTACAACGA TCCACGCC | 14358 |
| 6242 | GUGGAGCA G UCCUCAUU | 5610 | AATGAGGA GGCTAGCTACAACGA TGCTCCAC | 14359 |
| 6236 | CAGUCCUC A UUGAUCCA | 5611 | TGGATCAA GGCTAGCTACAACGA GAGGACTG | 14360 |
| 6232 | CCUCAUUG A UCCACUGA | 5612 | TCAGTGGA GGCTAGCTACAACGA CAATGAGG | 14361 |
| 6228 | AUUGAUCC A CUGAUGGA | 5613 | TCCATCAG GGCTAGCTACAACGA GGATCAAT | 14362 |
| 6224 | AUCCACUG A UGGAGCCU | 5614 | AGGCTCCA GGCTAGCTACAACGA CAGTGGAT | 14363 |
| 6219 | CUGAUGGA G CCUCCUCA | 5615 | TGAGGAGG GGCTAGCTACAACGA TCCATCAG | 14364 |
| 6210 | CCUCCUCA G CAGCUGAG | 5616 | CTCAGCTG GGCTAGCTACAACGA TGAGGAGG | 14365 |
| 6207 | CCUCAGCA G CUGAGUGA | 5617 | TCACTCAG GGCTAGCTACAACGA TGCTGAGG | 14366 |
| 6202 | GCAGCUGA G UGAUGGUG | 5618 | CACCATCA GGCTAGCTACAACGA TCAGCTGC | 14367 |
| 6199 | GCUGAGUG A UGGUGAGG | 5619 | CCTCACCA GGCTAGCTACAACGA CACTCAGC | 14368 |
| 6196 | GAGUGAUG G UGAGGCUG | 5620 | CAGCCTCA GGCTAGCTACAACGA CATCACTC | 14369 |
| 6191 | AUGGUGAG G CUGGAGAG | 5621 | CTCTCCAG GGCTAGCTACAACGA CTCACCAT | 14370 |
| 6181 | UGGAGAGG A UUUGUGUG | 5622 | CACACAAA GGCTAGCTACAACGA CCTCTCCA | 14371 |
| 6177 | GAGGAUUU G UGUGACGC | 5623 | GCGTCACA GGCTAGCTACAACGA AAATCCTC | 14372 |
| 6175 | GGAUUUGU G UGACGCGC | 5624 | GCGCGTCA GGCTAGCTACAACGA ACAAATCC | 14373 |
| 6172 | UUUGUGUG A CGCGCGCC | 5625 | GGCGCGCG GGCTAGCTACAACGA CACACAAA | 14374 |
| 6170 | UGUGUGAC G CGCGCCGC | 5626 | GCGGCGCG GGCTAGCTACAACGA GTCACACA | 14375 |
| 6168 | UGUGACGC G CGCCGCUG | 5627 | CAGCGGCG GGCTAGCTACAACGA GCGTCACA | 14376 |
| 6166 | UGACGCGC G CCGCUGCG | 5628 | CGCAGCGG GGCTAGCTACAACGA GCGCGTCA | 14377 |
| 6163 | CGCGCGCC G CUGCGUCG | 5629 | CGACGCAG GGCTAGCTACAACGA GGCGCGCG | 14378 |
| 6160 | GCGCCGCU G CGUCGCUC | 5630 | GAGCGACG GGCTAGCTACAACGA AGCGGCGC | 14379 |
| 6158 | GCCGCUGC G UCGCUCUC | 5631 | GAGAGCGA GGCTAGCTACAACGA GCAGCGGC | 14380 |
| 6155 | GCUGCGUC G CUCUCAGG | 5632 | CCTGAGAG GGCTAGCTACAACGA GACGCAGC | 14381 |
| 6147 | GCUCUCAG G CACAUAGU | 5633 | ACTATGTG GGCTAGCTACAACGA CTGAGAGC | 14382 |
| 6145 | UCUCAGGC A CAUAGUGC | 5634 | GCACTATG GGCTAGCTACAACGA GCCTGAGA | 14383 |
| 6143 | UCAGGCAC A UAGUGCGU | 5635 | ACGCACTA GGCTAGCTACAACGA GTGCCTGA | 14384 |
| 6140 | GGCACAUA G UGCGUGGG | 5636 | CCCACGCA GGCTAGCTACAACGA TATGTGCC | 14385 |
| 6138 | CACAUAGU G CGUGGGGG | 5637 | CCCCCACG GGCTAGCTACAACGA ACTATGTG | 14386 |
| 6136 | CAUAGUGC G UGGGGGAG | 5638 | CTCCCCCA GGCTAGCTACAACGA GCACTATG | 14387 |
| 6127 | UGGGGGAG A CAUGGUUG | 5639 | CAACCATG GGCTAGCTACAACGA CTCCCCCA | 14388 |
| 6125 | GGGGAGAC A UGGUUGCC | 5640 | GGCAACCA GGCTAGCTACAACGA GTCTCCCC | 14389 |
| 6122 | GAGACAUG G UUGCCCCG | 5641 | CGGGGCAA GGCTAGCTACAACGA CATGTCTC | 14390 |
| 6119 | ACAUGGUU G CCCCGCGA | 5642 | TCGCGGGG GGCTAGCTACAACGA AACCATGT | 14391 |
| 6114 | GUUGCCCC G CGAAGCGA | 5643 | TCGCTTCG GGCTAGCTACAACGA GGGGCAAC | 14392 |
| 6109 | CCCGCGAA G CGAACGCU | 5644 | AGCGTTCG GGCTAGCTACAACGA TTCGCGGG | 14393 |
| 6105 | CGAAGCGA A CGCUAUCA | 5645 | TGATAGCG GGCTAGCTACAACGA TCGCTTCG | 14394 |
| 6103 | AAGCGAAC G CUAUCAGC | 5646 | GCTGATAG GGCTAGCTACAACGA GTTCGCTT | 14395 |
| 6100 | CGAACGCU A UCAGCCGA | 5647 | TCGGCTGA GGCTAGCTACAACGA AGCGTTCG | 14396 |
| 6096 | CGCUAUCA G CCGAUUCA | 5648 | TGAATCGG GGCTAGCTACAACGA TGATAGCG | 14397 |
| 6092 | AUCAGCCG A UUCAUCCA | 5649 | TGGATGAA GGCTAGCTACAACGA CGGCTGAT | 14398 |
| 6088 | GCCGAUUC A UCCACUGC | 5650 | GCAGTGGA GGCTAGCTACAACGA GAATCGGC | 14399 |
| 6084 | AUUCAUCC A CUGCACAG | 5651 | CTGTGCAG GGCTAGCTACAACGA GGATGAAT | 14400 |
| 6081 | CAUCCACU G CACAGCGC | 5652 | GCGCTGTG GGCTAGCTACAACGA AGTGGATG | 14401 |
| 6079 | UCCACUGC A CAGCGCCC | 5653 | GGGCGCTG GGCTAGCTACAACGA GCAGTGGA | 14402 |
| 6076 | ACUGCACA G CGCCCUCU | 5654 | AGAGGGCG GGCTAGCTACAACGA TGTGCAGT | 14403 |
| 6074 | UGCACAGC G CCCUCUCC | 5655 | GGAGAGGG GGCTAGCTACAACGA GCTGTGCA | 14404 |
| 6062 | UCUCCUGG G CCCACAUG | 5656 | CATGTGGG GGCTAGCTACAACGA CCAGGAGA | 14405 |
| 6058 | CUGGGCCC A CAUGCCGA | 5657 | TCGGCATG GGCTAGCTACAACGA GGGCCCAG | 14406 |
| 6056 | GGGCCCAC A UGCCGACG | 5658 | CGTCGGCA GGCTAGCTACAACGA GTGGGCCC | 14407 |
| 6054 | GCCCACAU G CCGACGCA | 5659 | TGCGTCGG GGCTAGCTACAACGA ATGTGGGC | 14408 |
| 6050 | ACAUGCCG A CGCAGUAU | 5660 | ATACTGCG GGCTAGCTACAACGA CGGCATGT | 14409 |
| 6048 | AUGCCGAC G CAGUAUCG | 5661 | CGATACTG GGCTAGCTACAACGA GTCGGCAT | 14410 |
| 6045 | CCGACGCA G UAUCGCUG | 5662 | CAGCGATA GGCTAGCTACAACGA TGCGTCGG | 14411 |
| 6043 | GACGCAGU A UCGCUGCG | 5663 | CGCAGCGA GGCTAGCTACAACGA ACTGCGTC | 14412 |
| 6040 | GCAGUAUC G CUGCGCAC | 5664 | GTGCGCAG GGCTAGCTACAACGA GATACTGC | 14413 |
| 6037 | GUAUCGCU G CGCACACC | 5665 | GGTGTGCG GGCTAGCTACAACGA AGCGATAC | 14414 |
| 6035 | AUCGCUGC G CACACCAC | 5666 | GTGGTGTG GGCTAGCTACAACGA GCAGCGAT | 14415 |
| 6033 | CGCUGCGC A CACCACCC | 5667 | GGGTGGTG GGCTAGCTACAACGA GCGCAGCG | 14416 |
| 6031 | CUGCGCAC A CCACCCCG | 5668 | CGGGGTGG GGCTAGCTACAACGA GTGCGCAG | 14417 |
| 6028 | CGCACACC A CCCCGACG | 5669 | CGTCGGGG GGCTAGCTACAACGA GGTGTGCG | 14418 |
| 6022 | CCACCCCG A CGACCAGG | 5670 | CCTGGTCG GGCTAGCTACAACGA CGGGGTGG | 14419 |
| 6019 | CCCCGACG A CCAGGGCG | 5671 | CGCCCTGG GGCTAGCTACAACGA CGTCGGGG | 14420 |
| 6013 | CGACCAGG G CGCCAGGA | 5672 | TCCTGGCG GGCTAGCTACAACGA CCTGGTCG | 14421 |
| 6011 | ACCAGGGC G CCAGGAGA | 5673 | TCTCCTGG GGCTAGCTACAACGA GCCCTGGT | 14422 |
| 5998 | GAGAGAGG A UGGCAGGG | 5674 | CCCTGCCA GGCTAGCTACAACGA CCTCTCTC | 14423 |
| 5995 | AGAGGAUG G CAGGGAGU | 5675 | ACTCCCTG GGCTAGCTACAACGA CATCCTCT | 14424 |
| 5988 | GGCAGGGA G UAAGUUGA | 5676 | TCAACTTA GGCTAGCTACAACGA TCCCTGCC | 14425 |
| 5984 | GGGAGUAA G UUGACCAG | 5677 | CTGGTCAA GGCTAGCTACAACGA TTACTCCC | 14426 |
| 5980 | GUAAGUUG A CCAGGUCC | 5678 | GGACCTGG GGCTAGCTACAACGA CAACTTAC | 14427 |
| 5975 | UUGACCAG G UCCUCGGU | 5679 | ACCGAGGA GGCTAGCTACAACGA CTGGTCAA | 14428 |
| 5968 | GGUCCUCG G UAGAAGGC | 5680 | GCCTTCTA GGCTAGCTACAACGA CGAGGACC | 14429 |
| 5961 | GGUAGAAG G CAUCUCCC | 5681 | GGGAGATG GGCTAGCTACAACGA CTTCTACC | 14430 |
| 5959 | UAGAAGGC A UCUCCCCG | 5682 | CGGGGAGA GGCTAGCTACAACGA GCCTTCTA | 14431 |
| 5951 | AUCUCCCC G CUCAUGAC | 5683 | GTCATGAG GGCTAGCTACAACGA GGGGAGAT | 14432 |
| 5947 | CCCCGCUC A UGACCUUG | 5684 | CAAGGTCA GGCTAGCTACAACGA GAGCGGGG | 14433 |
| 5944 | CGCUCAUG A CCUUGAAG | 5685 | CTTCAAGG GGCTAGCTACAACGA CATGAGCG | 14434 |
| 5935 | CCUUGAAG G CCACGAGA | 5686 | TCTCGTGG GGCTAGCTACAACGA CTTCAAGG | 14435 |
| 5932 | UGAAGGCC A CGAGAGCA | 5687 | TGCTCTCG GGCTAGCTACAACGA GGCCTTCA | 14436 |
| 5926 | CCACGAGA G CACCCGCC | 5688 | GGCGGGTG GGCTAGCTACAACGA TCTCGTGG | 14437 |
| 5924 | ACGAGAGC A CCCGCCAC | 5689 | GTGGCGGG GGCTAGCTACAACGA GCTCTCGT | 14438 |
| 5920 | GAGCACCC G CCACUCCU | 5690 | AGGAGTGG GGCTAGCTACAACGA GGGTGCTC | 14439 |
| 5917 | CACCCGCC A CUCCUGCU | 5691 | AGCAGGAG GGCTAGCTACAACGA GGCGGGTG | 14440 |
| 5911 | CCACUCCU G CUCCAUAG | 5692 | CTATGGAG GGCTAGCTACAACGA AGGAGTGG | 14441 |
| 5906 | CCUGCUCC A UAGCCCGC | 5693 | GCGGGCTA GGCTAGCTACAACGA GGAGCAGG | 14442 |
| 5903 | GCUCCAUA G CCCGCCAG | 5694 | CTGGCGGG GGCTAGCTACAACGA TATGGAGC | 14443 |
| 5899 | CAUAGCCC G CCAGAAUG | 5695 | CATTCTGG GGCTAGCTACAACGA GGGCTATG | 14444 |
| 5893 | CCGCCAGA A UGUCUACA | 5696 | TGTAGACA GGCTAGCTACAACGA TCTGGCGG | 14445 |
| 5891 | GCCAGAAU G UCUACAAG | 5697 | CTTGTAGA GGCTAGCTACAACGA ATTCTGGC | 14446 |
| 5887 | GAAUGUCU A CAAGCACC | 5698 | GGTGCTTG GGCTAGCTACAACGA AGACATTC | 14447 |
| 5883 | GUCUACAA G CACCUUCC | 5699 | GGAAGGTG GGCTAGCTACAACGA TTGTAGAC | 14448 |
| 5881 | CUACAAGC A CCUUCCCA | 5700 | TGGGAAGG GGCTAGCTACAACGA GCTTGTAG | 14449 |
| 5870 | UUCCCAAG G CCUAUGCU | 5701 | AGCATAGG GGCTAGCTACAACGA CTTGGGAA | 14450 |
| 5866 | CAAGGCCU A UGCUGCCA | 5702 | TGGCAGCA GGCTAGCTACAACGA AGGCCTTG | 14451 |
| 5864 | AGGCCUAU G CUGCCAAC | 5703 | GTTGGCAG GGCTAGCTACAACGA ATAGGCCT | 14452 |
| 5861 | CCUAUGCU G CCAACAGC | 5704 | GCTGTTGG GGCTAGCTACAACGA AGCATAGG | 14453 |
| 5857 | UGCUGCCA A CAGCCGCG | 5705 | CGCGGCTG GGCTAGCTACAACGA TGGCAGCA | 14454 |
| 5854 | UGCCAACA G CCGCGCCA | 5706 | TGGCGCGG GGCTAGCTACAACGA TGTTGGCA | 14455 |
| 5851 | CAACAGCC G CGCCAGCG | 5707 | CGCTGGCG GGCTAGCTACAACGA GGCTGTTG | 14456 |
| 5849 | ACAGCCGC G CCAGCGAU | 5708 | ATCGCTGG GGCTAGCTACAACGA GCGGCTGT | 14457 |
| 5845 | CCGCGCCA G CGAUGCCG | 5709 | CGGCATCG GGCTAGCTACAACGA TGGCGCGG | 14458 |
| 5842 | CGCCAGCG A UGCCGGCG | 5710 | CGCCGGCA GGCTAGCTACAACGA CGCTGGCG | 14459 |
| 5840 | CCAGCGAU G CCGGCGCC | 5711 | GGCGCCGG GGCTAGCTACAACGA ATCGCTGG | 14460 |
| 5836 | CGAUGCCG G CGCCCACG | 5712 | CGTGGGCG GGCTAGCTACAACGA CGGCATCG | 14461 |
| 5834 | AUGCCGGC G CCCACGAA | 5713 | TTCGTGGG GGCTAGCTACAACGA GCCGGCAT | 14462 |
| 5830 | CGGCGCCC A CGAAGGCC | 5714 | GGCCTTCG GGCTAGCTACAACGA GGGCGCCG | 14463 |
| 5824 | CCACGAAG G CCGAAACG | 5715 | CGTTTCGG GGCTAGCTACAACGA CTTCGTGG | 14464 |
| 5818 | AGGCCGAA A CGGCUCUG | 5716 | CAGAGCCG GGCTAGCTACAACGA TTCGGCCT | 14465 |
| 5815 | CCGAAACG G CUCUGGGG | 5717 | CCCCAGAG GGCTAGCTACAACGA CGTTTCGG | 14466 |
| 5803 | UGGGGGGA G CGAGUUGG | 5718 | CCAACTCG GGCTAGCTACAACGA TCCCCCCA | 14467 |
| 5799 | GGGAGCGA G UUGGGCGG | 5719 | CCGCCCAA GGCTAGCTACAACGA TCGCTCCC | 14468 |
| 5794 | CGAGUUGG G CGGCCACC | 5720 | GGTGGCCG GGCTAGCTACAACGA CCAACTCG | 14469 |
| 5791 | GUUGGGCG G CCACCCAC | 5721 | GTGGGTGG GGCTAGCTACAACGA CGCCCAAC | 14470 |
| 5788 | GGGCGGCC A CCCACCCU | 5722 | AGGGTGGG GGCTAGCTACAACGA GGCCGCCC | 14471 |
| 5784 | GGCCACCC A CCCUCCCA | 5723 | TGGGAGGG GGCTAGCTACAACGA GGGTGGCC | 14472 |
| 5773 | CUCCCAAG A UGUUGAAC | 5724 | GTTCAACA GGCTAGCTACAACGA CTTGGGAG | 14473 |
| 5771 | CCCAAGAU G UUGAACAG | 5725 | CTGTTCAA GGCTAGCTACAACGA ATCTTGGG | 14474 |
| 5766 | GAUGUUGA A CAGGAGGG | 5726 | CCCTCCTG GGCTAGCTACAACGA TCAACATC | 14475 |
| 5758 | ACAGGAGG G UGCUUUGG | 5727 | CCAAAGCA GGCTAGCTACAACGA CCTCCTGT | 14476 |
| 5756 | AGGAGGGU G CUUUGGGU | 5728 | ACCCAAAG GGCTAGCTACAACGA ACCCTCCT | 14477 |
| 5749 | UGCUUUGG G UGGUGAGC | 5729 | GCTCACCA GGCTAGCTACAACGA CCAAAGCA | 14478 |
| 5746 | UUUGGGUG G UGAGCGGG | 5730 | CCCGCTCA GGCTAGCTACAACGA CACCCAAA | 14479 |
| 5742 | GGUGGUGA G CGGGCUGG | 5731 | CCAGCCCG GGCTAGCTACAACGA TCACCACC | 14480 |
| 5738 | GUGAGCGG G CUGGUGAU | 5732 | ATCACCAG GGCTAGCTACAACGA CCGCTCAC | 14481 |
| 5734 | GCGGGCUG G UGAUGGAG | 5733 | CTCCATCA GGCTAGCTACAACGA CAGCCCGC | 14482 |
| 5731 | GGCUGGUG A UGGAGGCU | 5734 | AGCCTCCA GGCTAGCTACAACGA CACCAGCC | 14483 |
| 5725 | UGAUGGAG G CUGUGAAU | 5735 | ATTCACAG GGCTAGCTACAACGA CTCCATCA | 14484 |
| 5722 | UGGAGGCU G UGAAUGCC | 5736 | GGCATTCA GGCTAGCTACAACGA AGCCTCCA | 14485 |
| 5718 | GGCUGUGA A UGCCAUCA | 5737 | TGATGGCA GGCTAGCTACAACGA TCACAGCC | 14486 |
| 5716 | CUGUGAAU G CCAUCAAU | 5738 | ATTGATGG GGCTAGCTACAACGA ATTCACAG | 14487 |
| 5713 | UGAAUGCC A UCAAUGAU | 5739 | ATCATTGA GGCTAGCTACAACGA GGCATTCA | 14488 |
| 5709 | UGCCAUCA A UGAUGCUA | 5740 | TAGCATCA GGCTAGCTACAACGA TGATGGCA | 14489 |
| 5706 | CAUCAAUG A UGCUAUCG | 5741 | CGATAGCA GGCTAGCTACAACGA CATTGATG | 14490 |
| 5704 | UCAAUGAU G CUAUCGCG | 5742 | CGCGATAG GGCTAGCTACAACGA ATCATTGA | 14491 |
| 5701 | AUGAUGCU A UCGCGGGG | 5743 | CCCCGCGA GGCTAGCTACAACGA AGCATCAT | 14492 |
| 5698 | AUGCUAUC G CGGGGUUC | 5744 | GAACCCCG GGCTAGCTACAACGA GATAGCAT | 14493 |
| 5693 | AUCGCGGG G UUCCCAGG | 5745 | CCTGGGAA GGCTAGCTACAACGA CCCGCGAT | 14494 |
| 5685 | GUUCCCAG G CAGAGUGG | 5746 | CCACTCTG GGCTAGCTACAACGA CTGGGAAC | 14495 |
| 5680 | CAGGCAGA G UGGACAAG | 5747 | CTTGTCCA GGCTAGCTACAACGA TCTGCCTG | 14496 |
| 5676 | CAGAGUGG A CAAGCCUG | 5748 | CAGGCTTG GGCTAGCTACAACGA CCACTCTG | 14497 |
| 5672 | GUGGACAA G CCUGCUAG | 5749 | CTAGCAGG GGCTAGCTACAACGA TTGTCCAC | 14498 |
| 5668 | ACAAGCCU G CUAGGUAC | 5750 | GTACCTAG GGCTAGCTACAACGA AGGCTTGT | 14499 |
| 5663 | CCUGCUAG G UACUGUAU | 5751 | ATACAGTA GGCTAGCTACAACGA CTAGCAGG | 14500 |
| 5661 | UGCUAGGU A CUGUAUCC | 5752 | GGATACAG GGCTAGCTACAACGA ACCTAGCA | 14501 |
| 5658 | UAGGUACU G UAUCCCGC | 5753 | GCGGGATA GGCTAGCTACAACGA AGTACCTA | 14502 |
| 5656 | GGUACUGU A UCCCGCUG | 5754 | CAGCGGGA GGCTAGCTACAACGA ACAGTACC | 14503 |
| 5651 | UGUAUCCC G CUGAUGAA | 5755 | TTCATCAG GGCTAGCTACAACGA GGGATACA | 14504 |
| 5647 | UCCCGCUG A UGAAAUUC | 5756 | GAATTTCA GGCTAGCTACAACGA CAGCGGGA | 14505 |
| 5642 | CUGAUGAA A UUCCACAU | 5757 | ATGTGGAA GGCTAGCTACAACGA TTCATCAG | 14506 |
| 5637 | GAAAUUCC A CAUGUGCU | 5758 | AGCACATG GGCTAGCTACAACGA GGAATTTC | 14507 |
| 5635 | AAUUCCAC A UGUGCUUC | 5759 | GAAGCACA GGCTAGCTACAACGA GTGGAATT | 14508 |
| 5633 | UUCCACAU G UGCUUCGC | 5760 | GCGAAGCA GGCTAGCTACAACGA ATGTGGAA | 14509 |
| 5631 | CCACAUGU G CUUCGCCC | 5761 | GGGCGAAG GGCTAGCTACAACGA ACATGTGG | 14510 |
| 5626 | UGUGCUUC G CCCAGAAA | 5762 | TTTCTGGG GGCTAGCTACAACGA GAAGCACA | 14511 |
| 5617 | CCCAGAAA G CCUCAAGG | 5763 | CCTTGAGG GGCTAGCTACAACGA TTTCTGGG | 14512 |
| 5608 | CCUCAAGG G CUCGCCAC | 5764 | GTGGCGAG GGCTAGCTACAACGA CCTTGAGG | 14513 |
| 5604 | AAGGGCUC G CCACUUGG | 5765 | CCAAGTGG GGCTAGCTACAACGA GAGCCCTT | 14514 |
| 5601 | GGCUCGCC A CUUGGAUU | 5766 | AATCCAAG GGCTAGCTACAACGA GGCGAGCC | 14515 |
| 5595 | CCACUUGG A UUCCACCA | 5767 | TGGTGGAA GGCTAGCTACAACGA CCAAGTGG | 14516 |
| 5590 | UGGAUUCC A CCACGGGA | 5768 | TCCCGTGG GGCTAGCTACAACGA GGAATCCA | 14517 |
| 5587 | AUUCCACC A CGGGAGCA | 5769 | TGCTCCCG GGCTAGCTACAACGA GGTGGAAT | 14518 |
| 5581 | CCACGGGA G CAGCAGCC | 5770 | GGCTGCTG GGCTAGCTACAACGA TCCCGTGG | 14519 |
| 5578 | CGGGAGCA G CAGCCUCC | 5771 | GGAGGCTG GGCTAGCTACAACGA TGCTCCCG | 14520 |
| 5575 | GAGCAGCA G CCUCCGCU | 5772 | AGCGGAGG GGCTAGCTACAACGA TGCTGCTC | 14521 |
| 5569 | CAGCCUCC G CUUGGUUG | 5773 | CAACCAAG GGCTAGCTACAACGA GGAGGCTG | 14522 |
| 5564 | UCCGCUUG G UUGGUGGC | 5774 | GCCACCAA GGCTAGCTACAACGA CAAGCGGA | 14523 |
| 5560 | CUUGGUUG G UGGCUGUU | 5775 | AACAGCCA GGCTAGCTACAACGA CAACCAAG | 14524 |
| 5557 | GGUUGGUG G CUGUUUGC | 5776 | GCAAACAG GGCTAGCTACAACGA CACCAACC | 14525 |
| 5554 | UGGUGGCU G UUUGCAGC | 5777 | GCTGCAAA GGCTAGCTACAACGA AGCCACCA | 14526 |
| 5550 | GGCUGUUU G CAGCAAUC | 5778 | GATTGCTG GGCTAGCTACAACGA AAACAGCC | 14527 |
| 5547 | UGUUUGCA G CAAUCCGA | 5779 | TCGGATTG GGCTAGCTACAACGA TGCAAACA | 14528 |
| 5544 | UUGCAGCA A UCCGAGCG | 5780 | CGCTCGGA GGCTAGCTACAACGA TGCTGCAA | 14529 |
| 5538 | CAAUCCGA G CGCCUUCU | 5781 | AGAAGGCG GGCTAGCTACAACGA TCGGATTG | 14530 |
| 5536 | AUCCGAGC G CCUUCUGC | 5782 | GCAGAAGG GGCTAGCTACAACGA GCTCGGAT | 14531 |
| 5529 | CGCCUUCU G CUUGAACU | 5783 | AGTTCAAG GGCTAGCTACAACGA AGAAGGCG | 14532 |
| 5523 | CUGCUUGA A CUGCUCGG | 5784 | CCGAGCAG GGCTAGCTACAACGA TCAAGCAG | 14533 |
| 5520 | CUUGAACU G CUCGGCGA | 5785 | TCGCCGAG GGCTAGCTACAACGA AGTTCAAG | 14534 |
| 5515 | ACUGCUCG G CGAGCUGC | 5786 | GCAGCTCG GGCTAGCTACAACGA CGAGCAGT | 14535 |
| 5511 | CUCGGCGA G CUGCAUCC | 5787 | GGATGCAG GGCTAGCTACAACGA TCGCCGAG | 14536 |
| 5508 | GGCGAGCU G CAUCCCCU | 5788 | AGGGGATG GGCTAGCTACAACGA AGCTCGCC | 14537 |
| 5506 | CGAGCUGC A UCCCCUGU | 5789 | ACAGGGGA GGCTAGCTACAACGA GCAGCTCG | 14538 |
| 5499 | CAUCCCCU G UUCGAUGU | 5790 | ACATCGAA GGCTAGCTACAACGA AGGGGATG | 14539 |
| 5494 | CCUGUUCG A UGUAAGGG | 5791 | CCCTTACA GGCTAGCTACAACGA CGAACAGG | 14540 |
| 5492 | UGUUCGAU G UAAGGGAG | 5792 | CTCCCTTA GGCTAGCTACAACGA ATCGAACA | 14541 |
| 5483 | UAAGGGAG G UGUGAGGC | 5793 | GCCTCACA GGCTAGCTACAACGA CTCCCTTA | 14542 |
| 5481 | AGGGAGGU G UGAGGCAC | 5794 | GTGCCTCA GGCTAGCTACAACGA ACCTCCCT | 14543 |
| 5476 | GGUGUGAG G CACACUCC | 5795 | GGAGTGTG GGCTAGCTACAACGA CTCACACC | 14544 |
| 5474 | UGUGAGGC A CACUCCUC | 5796 | GAGGAGTG GGCTAGCTACAACGA GCCTCACA | 14545 |
| 5472 | UGAGGCAC A CUCCUCCA | 5797 | TGGAGGAG GGCTAGCTACAACGA GTGCCTCA | 14546 |
| 5464 | ACUCCUCC A UCUCAUCG | 5798 | CGATGAGA GGCTAGCTACAACGA GGAGGAGT | 14547 |
| 5459 | UCCAUCUC A UCGAACUC | 5799 | GAGTTCGA GGCTAGCTACAACGA GAGATGGA | 14548 |
| 5454 | CUCAUCGA A CUCCUGGU | 5800 | ACCAGGAG GGCTAGCTACAACGA TCGATGAG | 14549 |
| 5447 | AACUCCUG G UAGAGAGC | 5801 | GCTCTCTA GGCTAGCTACAACGA CAGGAGTT | 14550 |
| 5440 | GGUAGAGA G CCUCCCUG | 5802 | CAGGGAGG GGCTAGCTACAACGA TCTCTACC | 14551 |
| 5432 | GCCUCCCU G UCGGGGAU | 5803 | ATCCCCGA GGCTAGCTACAACGA AGGGAGGC | 14552 |
| 5425 | UGUCGGGG A UAACAGCC | 5804 | GGCTGTTA GGCTAGCTACAACGA CCCCGACA | 14553 |
| 5422 | CGGGGAUA A CAGCCGGC | 5805 | GCCGGCTG GGCTAGCTACAACGA TATCCCCG | 14554 |
| 5419 | GGAUAACA G CCGGCUUC | 5806 | GAAGCCGG GGCTAGCTACAACGA TGTTATCC | 14555 |
| 5415 | AACAGCCG G CUUCCCGG | 5807 | CCGGGAAG GGCTAGCTACAACGA CGGCTGTT | 14556 |
| 5406 | CUUCCCGG A CAAGAUGA | 5808 | TCATCTTG GGCTAGCTACAACGA CCGGGAAG | 14557 |
| 5401 | CGGACAAG A UGAUUCUG | 5809 | CAGAATCA GGCTAGCTACAACGA CTTGTCCG | 14558 |
| 5398 | ACAAGAUG A UUCUGCCC | 5810 | GGGCAGAA GGCTAGCTACAACGA CATCTTGT | 14559 |
| 5393 | AUGAUUCU G CCCACAAU | 5811 | ATTGTGGG GGCTAGCTACAACGA AGAATCAT | 14560 |
| 5389 | UUCUGCCC A CAAUGACC | 5812 | GGTCATTG GGCTAGCTACAACGA GGGCAGAA | 14561 |
| 5386 | UGCCCACA A UGACCACG | 5813 | CGTGGTCA GGCTAGCTACAACGA TGTGGGCA | 14562 |
| 5383 | CCACAAUG A CCACGCUG | 5814 | CAGCGTGG GGCTAGCTACAACGA CATTGTGG | 14563 |
| 5380 | CAAUGACC A CGCUGCCU | 5815 | AGGCAGCG GGCTAGCTACAACGA GGTCATTG | 14564 |
| 5378 | AUGACCAC G CUGCCUGU | 5816 | ACAGGCAG GGCTAGCTACAACGA GTGGTCAT | 14565 |
| 5375 | ACCACGCU G CCUGUCGU | 5817 | ACGACAGG GGCTAGCTACAACGA AGCGTGGT | 14566 |
| 5371 | CGCUGCCU G UCGUCAGG | 5818 | CCTGACGA GGCTAGCTACAACGA AGGCAGCG | 14567 |
| 5368 | UGCCUGUC G UCAGGCAA | 5819 | TTGCCTGA GGCTAGCTACAACGA GACAGGCA | 14568 |
| 5363 | GUCGUCAG G CAAUACGC | 5820 | GCGTATTG GGCTAGCTACAACGA CTGACGAC | 14569 |
| 5360 | GUCAGGCA A UACGCGGU | 5821 | ACCGCGTA GGCTAGCTACAACGA TGCCTGAC | 14570 |
| 5358 | CAGGCAAU A CGCGGUCA | 5822 | TGACCGCG GGCTAGCTACAACGA ATTGCCTG | 14571 |
| 5356 | GGCAAUAC G CGGUCAGA | 5823 | TCTGACCG GGCTAGCTACAACGA GTATTGCC | 14572 |
| 5353 | AAUACGCG G UCAGAGCU | 5824 | AGCTCTGA GGCTAGCTACAACGA CGCGTATT | 14573 |
| 5347 | CGGUCAGA G CUGCCAGG | 5825 | CCTGGCAG GGCTAGCTACAACGA TCTGACCG | 14574 |
| 5344 | UCAGAGCU G CCAGGACG | 5826 | CGTCCTGG GGCTAGCTACAACGA AGCTCTGA | 14575 |
| 5338 | CUGCCAGG A CGCCACCU | 5827 | AGGTGGCG GGCTAGCTACAACGA CCTGGCAG | 14576 |
| 5336 | GCCAGGAC G CCACCUAC | 5828 | GTAGGTGG GGCTAGCTACAACGA GTCCTGGC | 14577 |
| 5333 | AGGACGCC A CCUACUAG | 5829 | CTAGTAGG GGCTAGCTACAACGA GGCGTCCT | 14578 |
| 5329 | CGCCACCU A CUAGCACC | 5830 | GGTGCTAG GGCTAGCTACAACGA AGGTGGCG | 14579 |
| 5325 | ACCUACUA G CACCCAGG | 5831 | CCTGGGTG GGCTAGCTACAACGA TAGTAGGT | 14580 |
| 5323 | CUACUAGC A CCCAGGUG | 5832 | CACCTGGG GGCTAGCTACAACGA GCTAGTAG | 14581 |
| 5317 | GCACCCAG G UGCUGGUG | 5833 | CACCAGCA GGCTAGCTACAACGA CTGGGTGC | 14582 |
| 5315 | ACCCAGGU G CUGGUGAC | 5834 | GTCACCAG GGCTAGCTACAACGA ACCTGGGT | 14583 |
| 5311 | AGGUGCUG G UGACGACC | 5835 | GGTCGTCA GGCTAGCTACAACGA CAGCACCT | 14584 |
| 5308 | UGCUGGUG A CGACCUCC | 5836 | GGAGGTCG GGCTAGCTACAACGA CACCAGCA | 14585 |
| 5305 | UGGUGACG A CCUCCAGG | 5837 | CCTGGAGG GGCTAGCTACAACGA CGTCACCA | 14586 |
| 5297 | ACCUCCAG G UCAGCCGA | 5838 | TCGGCTGA GGCTAGCTACAACGA CTGGAGGT | 14587 |
| 5293 | CCAGGUCA G CCGACAUG | 5839 | CATGTCGG GGCTAGCTACAACGA TGACCTGG | 14588 |
| 5289 | GUCAGCCG A CAUGCAUG | 5840 | CATGCATG GGCTAGCTACAACGA CGGCTGAC | 14589 |
| 5287 | CAGCCGAC A UGCAUGUC | 5841 | GACATGCA GGCTAGCTACAACGA GTCGGCTG | 14590 |
| 5285 | GCCGACAU G CAUGUCAU | 5842 | ATGACATG GGCTAGCTACAACGA ATGTCGGC | 14591 |
| 5283 | CGACAUGC A UGUCAUGA | 5843 | TCATGACA GGCTAGCTACAACGA GCATGTCG | 14592 |
| 5281 | ACAUGCAU G UCAUGAUG | 5844 | CATCATGA GGCTAGCTACAACGA ATGCATGT | 14593 |
| 5278 | UGCAUGUC A UGAUGUAU | 5845 | ATACATCA GGCTAGCTACAACGA GACATGCA | 14594 |
| 5275 | AUGUCAUG A UGUAUUUG | 5846 | CAAATACA GGCTAGCTACAACGA CATGACAT | 14595 |
| 5273 | GUCAUGAU G UAUUUGGU | 5847 | ACCAAATA GGCTAGCTACAACGA ATCATGAC | 14596 |
| 5271 | CAUGAUGU A UUUGGUUA | 5848 | TAACCAAA GGCTAGCTACAACGA ACATCATG | 14597 |
| 5266 | UGUAUUUG G UUAUGGGG | 5849 | CCCCATAA GGCTAGCTACAACGA CAAATACA | 14598 |
| 5263 | AUUUGGUU A UGGGGUGU | 5850 | ACACCCCA GGCTAGCTACAACGA AACCAAAT | 14599 |
| 5258 | GUUAUGGG G UGUGUGAG | 5851 | CTCACACA GGCTAGCTACAACGA CCCATAAC | 14600 |
| 5256 | UAUGGGGU G UGUGAGGG | 5852 | CCCTCACA GGCTAGCTACAACGA ACCCCATA | 14601 |
| 5254 | UGGGGUGU G UGAGGGUG | 5853 | CACCCTCA GGCTAGCTACAACGA ACACCCCA | 14602 |
| 5248 | GUGUGAGG G UGACAUCA | 5854 | TGATGTCA GGCTAGCTACAACGA CCTCACAC | 14603 |
| 5245 | UGAGGGUG A CAUCAUUU | 5855 | AAATGATG GGCTAGCTACAACGA CACCCTCA | 14604 |
| 5243 | AGGGUGAC A UCAUUUUG | 5856 | CAAAATGA GGCTAGCTACAACGA GTCACCCT | 14605 |
| 5240 | GUGACAUC A UUUUGGAC | 5857 | GTCCAAAA GGCTAGCTACAACGA GATGTCAC | 14606 |
| 5233 | CAUUUUGG A CGGCUCCU | 5858 | AGGAGCCG GGCTAGCTACAACGA CCAAAATG | 14607 |
| 5230 | UUUGGACG G CUCCUAGC | 5859 | GCTAGGAG GGCTAGCTACAACGA CGTCCAAA | 14608 |
| 5223 | GGCUCCUA G CCUAUACA | 5860 | TGTATAGG GGCTAGCTACAACGA TAGGAGCC | 14609 |
| 5219 | CCUAGCCU A UACAGCAG | 5861 | CTGCTGTA GGCTAGCTACAACGA AGGCTAGG | 14610 |
| 5217 | UAGCCUAU A CAGCAGGG | 5862 | CCCTGCTG GGCTAGCTACAACGA ATAGGCTA | 14611 |
| 5214 | CCUAUACA G CAGGGGUG | 5863 | CACCCCTG GGCTAGCTACAACGA TGTATAGG | 14612 |
| 5208 | CAGCAGGG G UGUUGGCC | 5864 | GGCCAACA GGCTAGCTACAACGA CCCTGCTG | 14613 |
| 5206 | GCAGGGGU G UUGGCCCG | 5865 | CGGGCCAA GGCTAGCTACAACGA ACCCCTGC | 14614 |
| 5202 | GGGUGUUG G CCCGUGUA | 5866 | TACACGGG GGCTAGCTACAACGA CAACACCC | 14615 |
| 5198 | GUUGGCCC G UGUAGCGU | 5867 | ACGCTACA GGCTAGCTACAACGA GGGCCAAC | 14616 |
| 5196 | UGGCCCGU G UAGCGUAG | 5868 | CTACGCTA GGCTAGCTACAACGA ACGGGCCA | 14617 |
| 5193 | CCCGUGUA G CGUAGGCU | 5869 | AGCCTACG GGCTAGCTACAACGA TACACGGG | 14618 |
| 5191 | CGUGUAGC G UAGGCUUU | 5870 | AAAGCCTA GGCTAGCTACAACGA GCTACACG | 14619 |
| 5187 | UAGCGUAG G CUUUAGCC | 5871 | GGCTAAAG GGCTAGCTACAACGA CTACGCTA | 14620 |
| 5181 | AGGCUUUA G CCGUGUGA | 5872 | TCACACGG GGCTAGCTACAACGA TAAAGCCT | 14621 |
| 5178 | CUUUAGCC G UGUGAGAC | 5873 | GTCTCACA GGCTAGCTACAACGA GGCTAAAG | 14622 |
| 5176 | UUAGCCGU G UGAGACAC | 5874 | GTGTCTCA GGCTAGCTACAACGA ACGGCTAA | 14623 |
| 5171 | CGUGUGAG A CACUUCCA | 5875 | TGGAAGTG GGCTAGCTACAACGA CTCACACG | 14624 |
| 5169 | UGUGAGAC A CUUCCACA | 5876 | TGTGGAAG GGCTAGCTACAACGA GTCTCACA | 14625 |
| 5163 | ACACUUCC A CAUUUGAU | 5877 | ATCAAATG GGCTAGCTACAACGA GGAAGTGT | 14626 |
| 5161 | ACUUCCAC A UUUGAUCC | 5878 | GGATCAAA GGCTAGCTACAACGA GTGGAAGT | 14627 |
| 5156 | CACAUUUG A UCCCACGA | 5879 | TCGTGGGA GGCTAGCTACAACGA CAAATGTG | 14628 |
| 5151 | UUGAUCCC A CGAUGGGG | 5880 | CCCCATCG GGCTAGCTACAACGA GGGATCAA | 14629 |
| 5148 | AUCCCACG A UGGGGGUG | 5881 | CACCCCCA GGCTAGCTACAACGA CGTGGGAT | 14630 |
| 5142 | CGAUGGGG G UGGAGCCU | 5882 | AGGCTCCA GGCTAGCTACAACGA CCCCATCG | 14631 |
| 5137 | GGGGUGGA G CCUGAGCC | 5883 | GGCTCAGG GGCTAGCTACAACGA TCCACCCC | 14632 |
| 5131 | GAGCCUGA G CCCUGGCG | 5884 | CGCCAGGG GGCTAGCTACAACGA TCAGGCTC | 14633 |
| 5125 | GAGCCCUG G CGCACACU | 5885 | AGTGTGCG GGCTAGCTACAACGA CAGGGCTC | 14634 |
| 5123 | GCCCUGGC G CACACUGU | 5886 | ACAGTGTG GGCTAGCTACAACGA GCCAGGGC | 14635 |
| 5121 | CCUGGCGC A CACUGUGG | 5887 | CCACAGTG GGCTAGCTACAACGA GCGCCAGG | 14636 |
| 5119 | UGGCGCAC A CUGUGGCU | 5888 | AGCCACAG GGCTAGCTACAACGA GTGCGCCA | 14637 |
| 5116 | CGCACACU G UGGCUUGG | 5889 | CCAAGCCA GGCTAGCTACAACGA AGTGTGCG | 14638 |
| 5113 | ACACUGUG G CUUGGUAU | 5890 | ATACCAAG GGCTAGCTACAACGA CACAGTGT | 14639 |
| 5108 | GUGGCUUG G UAUGCUAC | 5891 | GTAGCATA GGCTAGCTACAACGA CAAGCCAC | 14640 |
| 5106 | GGCUUGGU A UGCUACCA | 5892 | TGGTAGCA GGCTAGCTACAACGA ACCAAGCC | 14641 |
| 5104 | CUUGGUAU G CUACCAGG | 5893 | CCTGGTAG GGCTAGCTACAACGA ATACCAAG | 14642 |
| 5101 | GGUAUGCU A CCAGGUAG | 5894 | CTACCTGG GGCTAGCTACAACGA AGCATACC | 14643 |
| 5096 | GCUACCAG G UAGGGGAG | 5895 | CTCCCCTA GGCTAGCTACAACGA CTGGTAGC | 14644 |
| 5087 | UAGGGGAG G UUUUCUCC | 5896 | GGAGAAAA GGCTAGCTACAACGA CTCCCCTA | 14645 |
| 5077 | UUUCUCCU G CCUGCUUG | 5897 | CAAGCAGG GGCTAGCTACAACGA AGGAGAAA | 14646 |
| 5073 | UCCUGCCU G CUUGGUCU | 5898 | AGACCAAG GGCTAGCTACAACGA AGGCAGGA | 14647 |
| 5068 | CCUGCUUG G UCUGGGAC | 5899 | GTCCCAGA GGCTAGCTACAACGA CAAGCAGG | 14648 |
| 5061 | GGUCUGGG A CAAGAAGU | 5900 | ACTTCTTG GGCTAGCTACAACGA CCCAGACC | 14649 |
| 5054 | GACAAGAA G UGGGCAUC | 5901 | GATGCCCA GGCTAGCTACAACGA TTCTTGTC | 14650 |
| 5050 | AGAAGUGG G CAUCUAUG | 5902 | CATAGATG GGCTAGCTACAACGA CCACTTCT | 14651 |
| 5048 | AAGUGGGC A UCUAUGUG | 5903 | CACATAGA GGCTAGCTACAACGA GCCCACTT | 14652 |
| 5044 | GGGCAUCU A UGUGGGUG | 5904 | CACCCACA GGCTAGCTACAACGA AGATGCCC | 14653 |
| 5042 | GCAUCUAU G UGGGUGAG | 5905 | CTCACCCA GGCTAGCTACAACGA ATAGATGC | 14654 |
| 5038 | CUAUGUGG G UGAGGCCU | 5906 | AGGCCTCA GGCTAGCTACAACGA CCACATAG | 14655 |
| 5033 | UGGGUGAG G CCUGUGAA | 5907 | TTCACAGG GGCTAGCTACAACGA CTCACCCA | 14656 |
| 5029 | UGAGGCCU G UGAAGACA | 5908 | TGTCTTCA GGCTAGCTACAACGA AGGCCTCA | 14657 |
| 5023 | CUGUGAAG A CACCCUCC | 5909 | GGAGGGTG GGCTAGCTACAACGA CTTCACAG | 14658 |
| 5021 | GUGAAGAC A CCCUCCCA | 5910 | TGGGAGGG GGCTAGCTACAACGA GTCTTCAC | 14659 |
| 5010 | CUCCCAGA A CUCCAGAU | 5911 | ATCTGGAG GGCTAGCTACAACGA TCTGGGAG | 14660 |
| 5003 | AACUCCAG A UGGUCCUG | 5912 | CAGGACCA GGCTAGCTACAACGA CTGGAGTT | 14661 |
| 5000 | UCCAGAUG G UCCUGGCA | 5913 | TGCCAGGA GGCTAGCTACAACGA CATCTGGA | 14662 |
| 4994 | UGGUCCUG G CAGAAGGG | 5914 | CCCTTCTG GGCTAGCTACAACGA CAGGACCA | 14663 |
| 4986 | GCAGAAGG G CAACCCUG | 5915 | CAGGGTTG GGCTAGCTACAACGA CCTTCTGC | 14664 |
| 4983 | GAAGGGCA A CCCUGGUG | 5916 | CACCAGGG GGCTAGCTACAACGA TGCCCTTC | 14665 |
| 4977 | CAACCCUG G UGUAUUUA | 5917 | TAAATACA GGCTAGCTACAACGA CAGGGTTG | 14666 |
| 4975 | ACCCUGGU G UAUUUAGG | 5918 | CCTAAATA GGCTAGCTACAACGA ACCAGGGT | 14667 |
| 4973 | CCUGGUGU A UUUAGGUA | 5919 | TACCTAAA GGCTAGCTACAACGA ACACCAGG | 14668 |
| 4967 | GUAUUUAG G UAAGCCCG | 5920 | CGGGCTTA GGCTAGCTACAACGA CTAAATAC | 14669 |
| 4963 | UUAGGUAA G CCCGCAAC | 5921 | GTTGCGGG GGCTAGCTACAACGA TTACCTAA | 14670 |
| 4959 | GUAAGCCC G CAACCUAA | 5922 | TTAGGTTG GGCTAGCTACAACGA GGGCTTAC | 14671 |
| 4956 | AGCCCGCA A CCUAACGG | 5923 | CCGTTAGG GGCTAGCTACAACGA TGCGGGCT | 14672 |
| 4951 | GCAACCUA A CGGAGGUC | 5924 | GACCTCCG GGCTAGCTACAACGA TAGGTTGC | 14673 |
| 4945 | UAACGGAG G UCUCGGCG | 5925 | CGCCGAGA GGCTAGCTACAACGA CTCCGTTA | 14674 |
| 4939 | AGGUCUCG G CGGGCGUG | 5926 | CACGCCCG GGCTAGCTACAACGA CGAGACCT | 14675 |
| 4935 | CUCGGCGG G CGUGAGCU | 5927 | AGCTCACG GGCTAGCTACAACGA CCGCCGAG | 14676 |
| 4933 | CGGCGGGC G UGAGCUCG | 5928 | CGAGCTCA GGCTAGCTACAACGA GCCCGCCG | 14677 |
| 4929 | GGGCGUGA G CUCGUACC | 5929 | GGTACGAG GGCTAGCTACAACGA TCACGCCC | 14678 |
| 4925 | GUGAGCUC G UACCAAGC | 5930 | GCTTGGTA GGCTAGCTACAACGA GAGCTCAC | 14679 |
| 4923 | GAGCUCGU A CCAAGCAC | 5931 | GTGCTTGG GGCTAGCTACAACGA ACGAGCTC | 14680 |
| 4918 | CGUACCAA G CACAUCCC | 5932 | GGGATGTG GGCTAGCTACAACGA TTGGTACG | 14681 |
| 4916 | UACCAAGC A CAUCCCGC | 5933 | GCGGGATG GGCTAGCTACAACGA GCTTGGTA | 14682 |
| 4914 | CCAAGCAC A UCCCGCGU | 5934 | ACGCGGGA GGCTAGCTACAACGA GTGCTTGG | 14683 |
| 4909 | CACAUCCC G CGUCAUAG | 5935 | CTATGACG GGCTAGCTACAACGA GGGATGTG | 14684 |
| 4907 | CAUCCCGC G UCAUAGCA | 5936 | TGCTATGA GGCTAGCTACAACGA GCGGGATG | 14685 |
| 4904 | CCCGCGUC A UAGCACUC | 5937 | GAGTGCTA GGCTAGCTACAACGA GACGCGGG | 14686 |
| 4901 | GCGUCAUA G CACUCACA | 5938 | TGTGAGTG GGCTAGCTACAACGA TATGACGC | 14687 |
| 4899 | GUCAUAGC A CUCACACA | 5939 | TGTGTGAG GGCTAGCTACAACGA GCTATGAC | 14688 |
| 4895 | UAGCACUC A CACAGGAC | 5940 | GTCCTGTG GGCTAGCTACAACGA GAGTGCTA | 14689 |
| 4893 | GCACUCAC A CAGGACCG | 5941 | CGGTCCTG GGCTAGCTACAACGA GTGAGTGC | 14690 |
| 4888 | CACACAGG A CCGAGGAG | 5942 | CTCCTCGG GGCTAGCTACAACGA CCTGTGTG | 14691 |
| 4880 | ACCGAGGA G UCGAACAU | 5943 | ATGTTCGA GGCTAGCTACAACGA TCCTCGGT | 14692 |
| 4875 | GGAGUCGA A CAUGCCCG | 5944 | CGGGCATG GGCTAGCTACAACGA TCGACTCC | 14693 |
| 4873 | AGUCGAAC A UGCCCGAA | 5945 | TTCGGGCA GGCTAGCTACAACGA GTTCGACT | 14694 |
| 4871 | UCGAACAU G CCCGAAGG | 5946 | CCTTCGGG GGCTAGCTACAACGA ATGTTCGA | 14695 |
| 4863 | GCCCGAAG G CCGCUCUC | 5947 | GAGAGCGG GGCTAGCTACAACGA CTTCGGGC | 14696 |
| 4860 | CGAAGGCC G CUCUCCUG | 5948 | CAGGAGAG GGCTAGCTACAACGA GGCCTTCG | 14697 |
| 4849 | CUCCUGGA G UCACAAAC | 5949 | GTTTGTGA GGCTAGCTACAACGA TCCAGGAG | 14698 |
| 4846 | CUGGAGUC A CAAACCUG | 5950 | CAGGTTTG GGCTAGCTACAACGA GACTCCAG | 14699 |
| 4842 | AGUCACAA A CCUGUAUA | 5951 | TATACAGG GGCTAGCTACAACGA TTGTGACT | 14700 |
| 4838 | ACAAACCU G UAUAUGCC | 5952 | GGCATATA GGCTAGCTACAACGA AGGTTTGT | 14701 |
| 4836 | AAACCUGU A UAUGCCUC | 5953 | GAGGCATA GGCTAGCTACAACGA ACAGGTTT | 14702 |
| 4834 | ACCUGUAU A UGCCUCUC | 5954 | GAGAGGCA GGCTAGCTACAACGA ATACAGGT | 14703 |
| 4832 | CUGUAUAU G CCUCUCCU | 5955 | AGGAGAGG GGCTAGCTACAACGA ATATACAG | 14704 |
| 4823 | CCUCUCCU G CCCCUACC | 5956 | GGTAGGGG GGCTAGCTACAACGA AGGAGAGG | 14705 |
| 4817 | CUGCCCCU A CCGGUCCU | 5957 | AGGACCGG GGCTAGCTACAACGA AGGGGCAG | 14706 |
| 4813 | CCCUACCG G UCCUACCU | 5958 | AGGTAGGA GGCTAGCTACAACGA CGGTAGGG | 14707 |
| 4808 | CCGGUCCU A CCUCGCCU | 5959 | AGGCGAGG GGCTAGCTACAACGA AGGACCGG | 14708 |
| 4803 | CCUACCUC G CCUCUGCG | 5960 | CGCAGAGG GGCTAGCTACAACGA GAGGTAGG | 14709 |
| 4797 | UCGCCUCU G CGAGCGGG | 5961 | CCCGCTCG GGCTAGCTACAACGA AGAGGCGA | 14710 |
| 4793 | CUCUGCGA G CGGGACAC | 5962 | GTGTCCCG GGCTAGCTACAACGA TCGCAGAG | 14711 |
| 4788 | CGAGCGGG A CACUGCGU | 5963 | ACGCAGTG GGCTAGCTACAACGA CCCGCTCG | 14712 |
| 4786 | AGCGGGAC A CUGCGUCU | 5964 | AGACGCAG GGCTAGCTACAACGA GTCCCGCT | 14713 |
| 4783 | GGGACACU G CGUCUUGG | 5965 | CCAAGACG GGCTAGCTACAACGA AGTGTCCC | 14714 |
| 4781 | GACACUGC G UCUUGGGG | 5966 | CCCCAAGA GGCTAGCTACAACGA GCAGTGTC | 14715 |
| 4773 | GUCUUGGG G CACGGUCG | 5967 | CGACCGTG GGCTAGCTACAACGA CCCAAGAC | 14716 |
| 4771 | CUUGGGGC A CGGUCGUC | 5968 | GACGACCG GGCTAGCTACAACGA GCCCCAAG | 14717 |
| 4768 | GGGGCACG G UCGUCGUC | 5969 | GACGACGA GGCTAGCTACAACGA CGTGCCCC | 14718 |
| 4765 | GCACGGUC G UCGUCUCA | 5970 | TGAGACGA GGCTAGCTACAACGA GACCGTGC | 14719 |
| 4762 | CGGUCGUC G UCUCAAUG | 5971 | CATTGAGA GGCTAGCTACAACGA GACGACCG | 14720 |
| 4756 | UCGUCUCA A UGGUGAAG | 5972 | CTTCACCA GGCTAGCTACAACGA TGAGACGA | 14721 |
| 4753 | UCUCAAUG G UGAAGGUA | 5973 | TACCTTCA GGCTAGCTACAACGA CATTGAGA | 14722 |
| 4747 | UGGUGAAG G UAGGGUCC | 5974 | GGACCCTA GGCTAGCTACAACGA CTTCACCA | 14723 |
| 4742 | AAGGUAGG G UCCAAGCU | 5975 | AGCTTGGA GGCTAGCTACAACGA CCTACCTT | 14724 |
| 4736 | GGGUCCAA G CUGAAGUC | 5976 | GACTTCAG GGCTAGCTACAACGA TTGGACCC | 14725 |
| 4730 | AAGCUGAA G UCGACUGU | 5977 | ACAGTCGA GGCTAGCTACAACGA TTCAGCTT | 14726 |
| 4726 | UGAAGUCG A CUGUUUGG | 5978 | CCAAACAG GGCTAGCTACAACGA CGACTTCA | 14727 |
| 4723 | AGUCGACU G UUUGGGUG | 5979 | CACCCAAA GGCTAGCTACAACGA AGTCGACT | 14728 |
| 4717 | CUGUUUGG G UGACACAU | 5980 | ATGTGTCA GGCTAGCTACAACGA CCAAACAG | 14729 |
| 4714 | UUUGGGUG A CACAUGUA | 5981 | TACATGTG GGCTAGCTACAACGA CACCCAAA | 14730 |
| 4712 | UGGGUGAC A CAUGUAUU | 5982 | AATACATG GGCTAGCTACAACGA GTCACCCA | 14731 |
| 4710 | GGUGACAC A UGUAUUAC | 5983 | GTAATACA GGCTAGCTACAACGA GTGTCACC | 14732 |
| 4708 | UGACACAU G UAUUACAG | 5984 | CTGTAATA GGCTAGCTACAACGA ATGTGTCA | 14733 |
| 4706 | ACACAUGU A UUACAGUC | 5985 | GACTGTAA GGCTAGCTACAACGA ACATGTGT | 14734 |
| 4703 | CAUGUAUU A CAGUCGAU | 5986 | ATCGACTG GGCTAGCTACAACGA AATACATG | 14735 |
| 4700 | GUAUUACA G UCGAUCAC | 5987 | GTGATCGA GGCTAGCTACAACGA TGTAATAC | 14736 |
| 4696 | UACAGUCG A UCACCGAG | 5988 | CTCGGTGA GGCTAGCTACAACGA CGACTGTA | 14737 |
| 4693 | AGUCGAUC A CCGAGUCA | 5989 | TGACTCGG GGCTAGCTACAACGA GATCGACT | 14738 |
| 4688 | AUCACCGA G UCAAAAUC | 5990 | GATTTTGA GGCTAGCTACAACGA TCGGTGAT | 14739 |
| 4682 | GAGUCAAA A UCGCCGGU | 5991 | ACCGGCGA GGCTAGCTACAACGA TTTGACTC | 14740 |
| 4679 | UCAAAAUC G CCGGUAUA | 5992 | TATACCGG GGCTAGCTACAACGA GATTTTGA | 14741 |
| 4675 | AAUCGCCG G UAUAGCCC | 5993 | GGGCTATA GGCTAGCTACAACGA CGGCGATT | 14742 |
| 4673 | UCGCCGGU A UAGCCCGU | 5994 | ACGGGCTA GGCTAGCTACAACGA ACCGGCGA | 14743 |
| 4670 | CCGGUAUA G CCCGUCAU | 5995 | ATGACGGG GGCTAGCTACAACGA TATACCGG | 14744 |
| 4666 | UAUAGCCC G UCAUUAGA | 5996 | TCTAATGA GGCTAGCTACAACGA GGGCTATA | 14745 |
| 4663 | AGCCCGUC A UUAGAGCG | 5997 | CGCTCTAA GGCTAGCTACAACGA GACGGGCT | 14746 |
| 4657 | UCAUUAGA G CGUCUGUU | 5998 | AACAGACG GGCTAGCTACAACGA TCTAATGA | 14747 |
| 4655 | AUUAGAGC G UCUGUUGC | 5999 | GCAACAGA GGCTAGCTACAACGA GCTCTAAT | 14748 |
| 4651 | GAGCGUCU G UUGCCACG | 6000 | CGTGGCAA GGCTAGCTACAACGA AGACGCTC | 14749 |
| 4648 | CGUCUGUU G CCACGACA | 6001 | TGTCGTGG GGCTAGCTACAACGA AACAGACG | 14750 |
| 4645 | CUGUUGCC A CGACAACG | 6002 | CGTTGTCG GGCTAGCTACAACGA GGCAACAG | 14751 |
| 4642 | UUGCCACG A CAACGACG | 6003 | CGTCGTTG GGCTAGCTACAACGA CGTGGCAA | 14752 |
| 4639 | CCACGACA A CGACGUCC | 6004 | GGACGTCG GGCTAGCTACAACGA TGTCGTGG | 14753 |
| 4636 | CGACAACG A CGUCCCCG | 6005 | CGGGGACG GGCTAGCTACAACGA CGTTGTCG | 14754 |
| 4634 | ACAACGAC G UCCCCGCU | 6006 | AGCGGGGA GGCTAGCTACAACGA GTCGTTGT | 14755 |
| 4628 | ACGUCCCC G CUGGCCGG | 6007 | CCGGCCAG GGCTAGCTACAACGA GGGGACGT | 14756 |
| 4624 | CCCCGCUG G CCGGUAUG | 6008 | CATACCGG GGCTAGCTACAACGA CAGCGGGG | 14757 |
| 4620 | GCUGGCCG G UAUGACGG | 6009 | CCGTCATA GGCTAGCTACAACGA CGGCCAGC | 14758 |
| 4618 | UGGCCGGU A UGACGGAC | 6010 | GTCCGTCA GGCTAGCTACAACGA ACCGGCCA | 14759 |
| 4615 | CCGGUAUG A CGGACACG | 6011 | CGTGTCCG GGCTAGCTACAACGA CATACCGG | 14760 |
| 4611 | UAUGACGG A CACGUCGA | 6012 | TCGACGTG GGCTAGCTACAACGA CCGTCATA | 14761 |
| 4609 | UGACGGAC A CGUCGAGA | 6013 | TCTCGACG GGCTAGCTACAACGA GTCCGTCA | 14762 |
| 4607 | ACGGACAC G UCGAGACC | 6014 | GGTCTCGA GGCTAGCTACAACGA GTGTCCGT | 14763 |
| 4601 | ACGUCGAG A CCCCGGUA | 6015 | TACCGGGG GGCTAGCTACAACGA CTCGACGT | 14764 |
| 4595 | AGACCCCG G UAAUACGC | 6016 | GCGTATTA GGCTAGCTACAACGA CGGGGTCT | 14765 |
| 4592 | CCCCGGUA A UACGCUAC | 6017 | GTAGCGTA GGCTAGCTACAACGA TACCGGGG | 14766 |
| 4590 | CCGGUAAU A CGCUACAG | 6018 | CTGTAGCG GGCTAGCTACAACGA ATTACCGG | 14767 |
| 4588 | GGUAAUAC G CUACAGCG | 6019 | CGCTGTAG GGCTAGCTACAACGA GTATTACC | 14768 |
| 4585 | AAUACGCU A CAGCGUUA | 6020 | TAACGCTG GGCTAGCTACAACGA AGCGTATT | 14769 |
| 4582 | ACGCUACA G CGUUAAGU | 6021 | ACTTAACG GGCTAGCTACAACGA TGTAGCGT | 14770 |
| 4580 | GCUACAGC G UUAAGUCC | 6022 | GGACTTAA GGCTAGCTACAACGA GCTGTAGC | 14771 |
| 4575 | AGCGUUAA G UCCGAGGC | 6023 | GCCTCGGA GGCTAGCTACAACGA TTAACGCT | 14772 |
| 4568 | AGUCCGAG G CCCGACAG | 6024 | CTGTCGGG GGCTAGCTACAACGA CTCGGACT | 14773 |
| 4563 | GAGGCCCG A CAGCUUUG | 6025 | CAAAGCTG GGCTAGCTACAACGA CGGGCCTC | 14774 |
| 4560 | GCCCGACA G CUUUGCAG | 6026 | CTGCAAAG GGCTAGCTACAACGA TGTCGGGC | 14775 |
| 4555 | ACAGCUUU G CAGCGAGC | 6027 | GCTCGCTG GGCTAGCTACAACGA AAAGCTGT | 14776 |
| 4552 | GCUUUGCA G CGAGCUCG | 6028 | CGAGCTCG GGCTAGCTACAACGA TGCAAAGC | 14777 |
| 4548 | UGCAGCGA G CUCGUCAC | 6029 | GTGACGAG GGCTAGCTACAACGA TCGCTGCA | 14778 |
| 4544 | GCGAGCUC G UCACAUUU | 6030 | AAATGTGA GGCTAGCTACAACGA GAGCTCGC | 14779 |
| 4541 | AGCUCGUC A CAUUUCUU | 6031 | AAGAAATG GGCTAGCTACAACGA GACGAGCT | 14780 |
| 4539 | CUCGUCAC A UUUCUUCU | 6032 | AGAAGAAA GGCTAGCTACAACGA GTGACGAG | 14781 |
| 4526 | UUCUUGGA A UGGCAGAA | 6033 | TTCTGCCA GGCTAGCTACAACGA TCCAAGAA | 14782 |
| 4523 | UUGGAAUG G CAGAAGAU | 6034 | ATCTTCTG GGCTAGCTACAACGA CATTCCAA | 14783 |
| 4516 | GGCAGAAG A UGAGAUGC | 6035 | GCATCTCA GGCTAGCTACAACGA CTTCTGCC | 14784 |
| 4511 | AAGAUGAG A UGCCUCCC | 6036 | GGGAGGCA GGCTAGCTACAACGA CTCATCTT | 14785 |
| 4509 | GAUGAGAU G CCUCCCCC | 6037 | GGGGGAGG GGCTAGCTACAACGA ATCTCATC | 14786 |
| 4495 | CCCCUUUG A UGGUCUCG | 6038 | CGAGACCA GGCTAGCTACAACGA CAAAGGGG | 14787 |
| 4492 | CUUUGAUG G UCUCGAUG | 6039 | CATCGAGA GGCTAGCTACAACGA CATCAAAG | 14788 |
| 4486 | UGGUCUCG A UGGGGAUG | 6040 | CATCCCCA GGCTAGCTACAACGA CGAGACCA | 14789 |
| 4480 | CGAUGGGG A UGGCUUUG | 6041 | CAAAGCCA GGCTAGCTACAACGA CCCCATCG | 14790 |
| 4477 | UGGGGAUG G CUUUGCCA | 6042 | TGGCAAAG GGCTAGCTACAACGA CATCCCCA | 14791 |
| 4472 | AUGGCUUU G CCAUAGAA | 6043 | TTCTATGG GGCTAGCTACAACGA AAAGCCAT | 14792 |
| 4469 | GCUUUGCC A UAGAAGGG | 6044 | CCCTTCTA GGCTAGCTACAACGA GGCAAAGC | 14793 |
| 4459 | AGAAGGGG A UCUCUCCG | 6045 | CGGAGAGA GGCTAGCTACAACGA CCCCTTCT | 14794 |
| 4450 | UCUCUCCG G UGUUGGAC | 6046 | GTCCAACA GGCTAGCTACAACGA CGGAGAGA | 14795 |
| 4448 | UCUCCGGU G UUGGACAA | 6047 | TTGTCCAA GGCTAGCTACAACGA ACCGGAGA | 14796 |
| 4443 | GGUGUUGG A CAAGGCUA | 6048 | TAGCCTTG GGCTAGCTACAACGA CCAACACC | 14797 |
| 4438 | UGGACAAG G CUAUCUCC | 6049 | GGAGATAG GGCTAGCTACAACGA CTTGTCCA | 14798 |
| 4435 | ACAAGGCU A UCUCCUCG | 6050 | CGAGGAGA GGCTAGCTACAACGA AGCCTTGT | 14799 |
| 4426 | UCUCCUCG A UGUUGGGA | 6051 | TCCCAACA GGCTAGCTACAACGA CGAGGAGA | 14800 |
| 4424 | UCCUCGAU G UUGGGAUG | 6052 | CATCCCAA GGCTAGCTACAACGA ATCGAGGA | 14801 |
| 4418 | AUGUUGGG A UGUGGCAC | 6053 | GTGCCACA GGCTAGCTACAACGA CCCAACAT | 14802 |
| 4416 | GUUGGGAU G UGGCACGG | 6054 | CCGTGCCA GGCTAGCTACAACGA ATCCCAAC | 14803 |
| 4413 | GGGAUGUG G CACGGUGA | 6055 | TCACCGTG GGCTAGCTACAACGA CACATCCC | 14804 |
| 4411 | GAUGUGGC A CGGUGACC | 6056 | GGTCACCG GGCTAGCTACAACGA GCCACATC | 14805 |
| 4408 | GUGGCACG G UGACCGAU | 6057 | ATCGGTCA GGCTAGCTACAACGA CGTGCCAC | 14806 |
| 4405 | GCACGGUG A CCGAUCCC | 6058 | GGGATCGG GGCTAGCTACAACGA CACCGTGC | 14807 |
| 4401 | GGUGACCG A UCCCGGAG | 6059 | CTCCGGGA GGCTAGCTACAACGA CGGTCACC | 14808 |
| 4392 | UCCCGGAG G CGUAGCGG | 6060 | CCGCTACG GGCTAGCTACAACGA CTCCGGGA | 14809 |
| 4390 | CCGGAGGC G UAGCGGUG | 6061 | CACCGCTA GGCTAGCTACAACGA GCCTCCGG | 14810 |
| 4387 | GAGGCGUA G CGGUGGCG | 6062 | CGCCACCG GGCTAGCTACAACGA TACGCCTC | 14811 |
| 4384 | GCGUAGCG G UGGCGAGC | 6063 | GCTCGCCA GGCTAGCTACAACGA CGCTACGC | 14812 |
| 4381 | UAGCGGUG G CGAGCACG | 6064 | CGTGCTCG GGCTAGCTACAACGA CACCGCTA | 14813 |
| 4377 | GGUGGCGA G CACGACGA | 6065 | TCGTCGTG GGCTAGCTACAACGA TCGCCACC | 14814 |
| 4375 | UGGCGAGC A CGACGAGC | 6066 | GCTCGTCG GGCTAGCTACAACGA GCTCGCCA | 14815 |
| 4372 | CGAGCACG A CGAGCCGC | 6067 | GCGGCTCG GGCTAGCTACAACGA CGTGCTCG | 14816 |
| 4368 | CACGACGA G CCGCGCUC | 6068 | GAGCGCGG GGCTAGCTACAACGA TCGTCGTG | 14817 |
| 4365 | GACGAGCC G CGCUCCAG | 6069 | CTGGAGCG GGCTAGCTACAACGA GGCTCGTC | 14818 |
| 4363 | CGAGCCGC G CUCCAGCC | 6070 | GGCTGGAG GGCTAGCTACAACGA GCGGCTCG | 14819 |
| 4357 | GCGCUCCA G CCGUCUCC | 6071 | GGAGACGG GGCTAGCTACAACGA TGGAGCGC | 14820 |
| 4354 | CUCCAGCC G UCUCCGCU | 6072 | AGCGGAGA GGCTAGCTACAACGA GGCTGGAG | 14821 |
| 4348 | CCGUCUCC G CUUGGUCC | 6073 | GGACCAAG GGCTAGCTACAACGA GGAGACGG | 14822 |
| 4343 | UCCGCUUG G UCCAGGAC | 6074 | GTCCTGGA GGCTAGCTACAACGA CAAGCGGA | 14823 |
| 4336 | GGUCCAGG A CUGUGCCG | 6075 | CGGCACAG GGCTAGCTACAACGA CCTGGACC | 14824 |
| 4333 | CCAGGACU G UGCCGAUG | 6076 | CATCGGCA GGCTAGCTACAACGA AGTCCTGG | 14825 |
| 4331 | AGGACUGU G CCGAUGCC | 6077 | GGCATCGG GGCTAGCTACAACGA ACAGTCCT | 14826 |
| 4327 | CUGUGCCG A UGCCCAAA | 6078 | TTTGGGCA GGCTAGCTACAACGA CGGCACAG | 14827 |
| 4325 | GUGCCGAU G CCCAAAAU | 6079 | ATTTTGGG GGCTAGCTACAACGA ATCGGCAC | 14828 |
| 4318 | UGCCCAAA A UGGAAGUC | 6080 | GACTTCCA GGCTAGCTACAACGA TTTGGGCA | 14829 |
| 4312 | AAAUGGAA G UCGAGUCA | 6081 | TGACTCGA GGCTAGCTACAACGA TTCCATTT | 14830 |
| 4307 | GAAGUCGA G UCAAUUGA | 6082 | TCAATTGA GGCTAGCTACAACGA TCGACTTC | 14831 |
| 4303 | UCGAGUCA A UUGAGUGG | 6083 | CCACTCAA GGCTAGCTACAACGA TGACTCGA | 14832 |
| 4298 | UCAAUUGA G UGGCACUC | 6084 | GAGTGCCA GGCTAGCTACAACGA TCAATTGA | 14833 |
| 4295 | AUUGAGUG G CACUCAUC | 6085 | GATGAGTG GGCTAGCTACAACGA CACTCAAT | 14834 |
| 4293 | UGAGUGGC A CUCAUCAC | 6086 | GTGATGAG GGCTAGCTACAACGA GCCACTCA | 14835 |
| 4289 | UGGCACUC A UCACACAU | 6087 | ATGTGTGA GGCTAGCTACAACGA GAGTGCCA | 14836 |
| 4286 | CACUCAUC A CACAUUAU | 6088 | ATAATGTG GGCTAGCTACAACGA GATGAGTG | 14837 |
| 4284 | CUCAUCAC A CAUUAUGA | 6089 | TCATAATG GGCTAGCTACAACGA GTGATGAG | 14838 |
| 4282 | CAUCACAC A UUAUGAUG | 6090 | CATCATAA GGCTAGCTACAACGA GTGTGATG | 14839 |
| 4279 | CACACAUU A UGAUGUCA | 6091 | TGACATCA GGCTAGCTACAACGA AATGTGTG | 14840 |
| 4276 | ACAUUAUG A UGUCAUAG | 6092 | CTATGACA GGCTAGCTACAACGA CATAATGT | 14841 |
| 4274 | AUUAUGAU G UCAUAGGC | 6093 | GCCTATGA GGCTAGCTACAACGA ATCATAAT | 14842 |
| 4271 | AUGAUGUC A UAGGCGCC | 6094 | GGCGCCTA GGCTAGCTACAACGA GACATCAT | 14843 |
| 4267 | UGUCAUAG G CGCCCCCA | 6095 | TGGGGGCG GGCTAGCTACAACGA CTATGACA | 14844 |
| 4265 | UCAUAGGC G CCCCCAGA | 6096 | TCTGGGGG GGCTAGCTACAACGA GCCTATGA | 14845 |
| 4256 | CCCCCAGA G CAACCACC | 6097 | GGTGGTTG GGCTAGCTACAACGA TCTGGGGG | 14846 |
| 4253 | CCAGAGCA A CCACCGUC | 6098 | GACGGTGG GGCTAGCTACAACGA TGCTCTGG | 14847 |
| 4250 | GAGCAACC A CCGUCGGC | 6099 | GCCGACGG GGCTAGCTACAACGA GGTTGCTC | 14848 |
| 4247 | CAACCACC G UCGGCAAG | 6100 | CTTGCCGA GGCTAGCTACAACGA GGTGGTTG | 14849 |
| 4243 | CACCGUCG G CAAGGAAC | 6101 | GTTCCTTG GGCTAGCTACAACGA CGACGGTG | 14850 |
| 4236 | GGCAAGGA A CUUGCCAU | 6102 | ATGGCAAG GGCTAGCTACAACGA TCCTTGCC | 14851 |
| 4232 | AGGAACUU G CCAUAGGU | 6103 | ACCTATGG GGCTAGCTACAACGA AAGTTCCT | 14852 |
| 4229 | AACUUGCC A UAGGUGGA | 6104 | TCCACCTA GGCTAGCTACAACGA GGCAAGTT | 14853 |
| 4225 | UGCCAUAG G UGGAGUAC | 6105 | GTACTCCA GGCTAGCTACAACGA CTATGGCA | 14854 |
| 4220 | UAGGUGGA G UACGUGAU | 6106 | ATCACGTA GGCTAGCTACAACGA TCCACCTA | 14855 |
| 4218 | GGUGGAGU A CGUGAUGG | 6107 | CCATCACG GGCTAGCTACAACGA ACTCCACC | 14856 |
| 4216 | UGGAGUAC G UGAUGGGG | 6108 | CCCCATCA GGCTAGCTACAACGA GTACTCCA | 14857 |
| 4213 | AGUACGUG A UGGGGGCG | 6109 | CGCCCCCA GGCTAGCTACAACGA CACGTACT | 14858 |
| 4207 | UGAUGGGG G CGCCCGUG | 6110 | CACGGGCG GGCTAGCTACAACGA CCCCATCA | 14859 |
| 4205 | AUGGGGGC G CCCGUGGU | 6111 | ACCACGGG GGCTAGCTACAACGA GCCCCCAT | 14860 |
| 4201 | GGGCGCCC G UGGUGAUG | 6112 | CATCACCA GGCTAGCTACAACGA GGGCGCCC | 14861 |
| 4198 | CGCCCGUG G UGAUGGUC | 6113 | GACCATCA GGCTAGCTACAACGA CACGGGCG | 14862 |
| 4195 | CCGUGGUG A UGGUCCUU | 6114 | AAGGACCA GGCTAGCTACAACGA CACCACGG | 14863 |
| 4192 | UGGUGAUG G UCCUUACC | 6115 | GGTAAGGA GGCTAGCTACAACGA CATCACCA | 14864 |
| 4186 | UGGUCCUU A CCCCAGUU | 6116 | AACTGGGG GGCTAGCTACAACGA AAGGACCA | 14865 |
| 4180 | UUACCCCA G UUCUGAUG | 6117 | CATCAGAA GGCTAGCTACAACGA TGGGGTAA | 14866 |
| 4174 | CAGUUCUG A UGUUAGGA | 6118 | TCCTAACA GGCTAGCTACAACGA CAGAACTG | 14867 |
| 4172 | GUUCUGAU G UUAGGAUC | 6119 | GATCCTAA GGCTAGCTACAACGA ATCAGAAC | 14868 |
| 4166 | AUGUUAGG A UCGACACC | 6120 | GGTGTCGA GGCTAGCTACAACGA CCTAACAT | 14869 |
| 4162 | UAGGAUCG A CACCGUGU | 6121 | ACACGGTG GGCTAGCTACAACGA CGATCCTA | 14870 |
| 4160 | GGAUCGAC A CCGUGUGC | 6122 | GCACACGG GGCTAGCTACAACGA GTCGATCC | 14871 |
| 4157 | UCGACACC G UGUGCCUU | 6123 | AAGGCACA GGCTAGCTACAACGA GGTGTCGA | 14872 |
| 4155 | GACACCGU G UGCCUUAG | 6124 | CTAAGGCA GGCTAGCTACAACGA ACGGTGTC | 14873 |
| 4153 | CACCGUGU G CCUUAGAC | 6125 | GTCTAAGG GGCTAGCTACAACGA ACACGGTG | 14874 |
| 4146 | UGCCUUAG A CAUAUACG | 6126 | CGTATATG GGCTAGCTACAACGA CTAAGGCA | 14875 |
| 4144 | CCUUAGAC A UAUACGCC | 6127 | GGCGTATA GGCTAGCTACAACGA GTCTAAGG | 14876 |
| 4142 | UUAGACAU A UACGCCCC | 6128 | GGGGCGTA GGCTAGCTACAACGA ATGTCTAA | 14877 |
| 4140 | AGACAUAU A CGCCCCAA | 6129 | TTGGGGCG GGCTAGCTACAACGA ATATGTCT | 14878 |
| 4138 | ACAUAUAC G CCCCAAAC | 6130 | GTTTGGGG GGCTAGCTACAACGA GTATATGT | 14879 |
| 4131 | CGCCCCAA A CCCUAAGG | 6131 | CCTTAGGG GGCTAGCTACAACGA TTGGGGCG | 14880 |
| 4123 | ACCCUAAG G UGGCGGUA | 6132 | TACCGCCA GGCTAGCTACAACGA CTTAGGGT | 14881 |
| 4120 | CUAAGGUG G CGGUAACG | 6133 | CGTTACCG GGCTAGCTACAACGA CACCTTAG | 14882 |
| 4117 | AGGUGGCG G UAACGGAC | 6134 | GTCCGTTA GGCTAGCTACAACGA CGCCACCT | 14883 |
| 4114 | UGGCGGUA A CGGACGGA | 6135 | TCCGTCCG GGCTAGCTACAACGA TACCGCCA | 14884 |
| 4110 | GGUAACGG A CGGAUUUA | 6136 | TAAATCCG GGCTAGCTACAACGA CCGTTACC | 14885 |
| 4106 | ACGGACGG A UUUAGGAC | 6137 | GTCCTAAA GGCTAGCTACAACGA CCGTCCGT | 14886 |
| 4099 | GAUUUAGG A CGAGCACU | 6138 | AGTGCTCG GGCTAGCTACAACGA CCTAAATC | 14887 |
| 4095 | UAGGACGA G CACUUUGU | 6139 | ACAAAGTG GGCTAGCTACAACGA TCGTCCTA | 14888 |
| 4093 | GGACGAGC A CUUUGUAC | 6140 | GTACAAAG GGCTAGCTACAACGA GCTCGTCC | 14889 |
| 4088 | AGCACUUU G UACCCUUG | 6141 | CAAGGGTA GGCTAGCTACAACGA AAAGTGCT | 14890 |
| 4086 | CACUUUGU A CCCUUGGG | 6142 | CCCAAGGG GGCTAGCTACAACGA ACAAAGTG | 14891 |
| 4078 | ACCCUUGG G CUGCAUAU | 6143 | ATATGCAG GGCTAGCTACAACGA CCAAGGGT | 14892 |
| 4075 | CUUGGGCU G CAUAUGCA | 6144 | TGCATATG GGCTAGCTACAACGA AGCCCAAG | 14893 |
| 4073 | UGGGCUGC A UAUGCAGC | 6145 | GCTGCATA GGCTAGCTACAACGA GCAGCCCA | 14894 |
| 4071 | GGCUGCAU A UGCAGCCG | 6146 | CGGCTGCA GGCTAGCTACAACGA ATGCAGCC | 14895 |
| 4069 | CUGCAUAU G CAGCCGGU | 6147 | ACCGGCTG GGCTAGCTACAACGA ATATGCAG | 14896 |
| 4066 | CAUAUGCA G CCGGUACC | 6148 | GGTACCGG GGCTAGCTACAACGA TGCATATG | 14897 |
| 4062 | UGCAGCCG G UACCUUAG | 6149 | CTAAGGTA GGCTAGCTACAACGA CGGCTGCA | 14898 |
| 4060 | CAGCCGGU A CCUUAGUG | 6150 | CACTAAGG GGCTAGCTACAACGA ACCGGCTG | 14899 |
| 4054 | GUACCUUA G UGCUCUUG | 6151 | CAAGAGCA GGCTAGCTACAACGA TAAGGTAC | 14900 |
| 4052 | ACCUUAGU G CUCUUGCC | 6152 | GGCAAGAG GGCTAGCTACAACGA ACTAAGGT | 14901 |
| 4046 | GUGCUCUU G CCGCUGCC | 6153 | GGCAGCGG GGCTAGCTACAACGA AAGAGCAC | 14902 |
| 4043 | CUCUUGCC G CUGCCAGU | 6154 | ACTGGCAG GGCTAGCTACAACGA GGCAAGAG | 14903 |
| 4040 | UUGCCGCU G CCAGUGGG | 6155 | CCCACTGG GGCTAGCTACAACGA AGCGGCAA | 14904 |
| 4036 | CGCUGCCA G UGGGAGCG | 6156 | CGCTCCCA GGCTAGCTACAACGA TGGCAGCG | 14905 |
| 4030 | CAGUGGGA G CGUGUAGG | 6157 | CCTACACG GGCTAGCTACAACGA TCCCACTG | 14906 |
| 4028 | GUGGGAGC G UGUAGGUG | 6158 | CACCTACA GGCTAGCTACAACGA GCTCCCAC | 14907 |
| 4026 | GGGAGCGU G UAGGUGGG | 6159 | CCCACCTA GGCTAGCTACAACGA ACGCTCCC | 14908 |
| 4022 | GCGUGUAG G UGGGCCAC | 6160 | GTGGCCCA GGCTAGCTACAACGA CTACACGC | 14909 |
| 4018 | GUAGGUGG G CCACUUGG | 6161 | CCAAGTGG GGCTAGCTACAACGA CCACCTAC | 14910 |
| 4015 | GGUGGGCC A CUUGGAAU | 6162 | ATTCCAAG GGCTAGCTACAACGA GGCCCACC | 14911 |
| 4008 | CACUUGGA A UGUCUGCG | 6163 | CGCAGACA GGCTAGCTACAACGA TCCAAGTG | 14912 |
| 4006 | CUUGGAAU G UCUGCGGU | 6164 | ACCGCAGA GGCTAGCTACAACGA ATTCCAAG | 14913 |
| 4002 | GAAUGUCU G CGGUACGG | 6165 | CCGTACCG GGCTAGCTACAACGA AGACATTC | 14914 |
| 3999 | UGUCUGCG G UACGGCUG | 6166 | CAGCCGTA GGCTAGCTACAACGA CGCAGACA | 14915 |
| 3997 | UCUGCGGU A CGGCUGGG | 6167 | CCCAGCCG GGCTAGCTACAACGA ACCGCAGA | 14916 |
| 3994 | GCGGUACG G CUGGGGGG | 6168 | CCCCCCAG GGCTAGCTACAACGA CGTACCGC | 14917 |
| 3984 | UGGGGGGG A CGAGUUGU | 6169 | ACAACTCG GGCTAGCTACAACGA CCCCCCCA | 14918 |
| 3980 | GGGGACGA G UUGUCCGU | 6170 | ACGGACAA GGCTAGCTACAACGA TCGTCCCC | 14919 |
| 3977 | GACGAGUU G UCCGUGAA | 6171 | TTCACGGA GGCTAGCTACAACGA AACTCGTC | 14920 |
| 3973 | AGUUGUCC G UGAAGACC | 6172 | GGTCTTCA GGCTAGCTACAACGA GGACAACT | 14921 |
| 3967 | CCGUGAAG A CCGGGGAC | 6173 | GTCCCCGG GGCTAGCTACAACGA CTTCACGG | 14922 |
| 3960 | GACCGGGG A CCGCAUGG | 6174 | CCATGCGG GGCTAGCTACAACGA CCCCGGTC | 14923 |
| 3957 | CGGGGACC G CAUGGUAG | 6175 | CTACCATG GGCTAGCTACAACGA GGTCCCCG | 14924 |
| 3955 | GGGACCGC A UGGUAGUU | 6176 | AACTACCA GGCTAGCTACAACGA GCGGTCCC | 14925 |
| 3952 | ACCGCAUG G UAGUUUCC | 6177 | GGAAACTA GGCTAGCTACAACGA CATGCGGT | 14926 |
| 3949 | GCAUGGUA G UUUCCAUA | 6178 | TATGGAAA GGCTAGCTACAACGA TACCATGC | 14927 |
| 3943 | UAGUUUCC A UAGACUCA | 6179 | TGAGTCTA GGCTAGCTACAACGA GGAAACTA | 14928 |
| 3939 | UUCCAUAG A CUCAACGG | 6180 | CCGTTGAG GGCTAGCTACAACGA CTATGGAA | 14929 |
| 3934 | UAGACUCA A CGGGUACA | 6181 | TGTACCCG GGCTAGCTACAACGA TGAGTCTA | 14930 |
| 3930 | CUCAACGG G UACAAAGU | 6182 | ACTTTGTA GGCTAGCTACAACGA CCGTTGAG | 14931 |
| 3928 | CAACGGGU A CAAAGUCC | 6183 | GGACTTTG GGCTAGCTACAACGA ACCCGTTG | 14932 |
| 3923 | GGUACAAA G UCCACCGC | 6184 | GCGGTGGA GGCTAGCTACAACGA TTTGTACC | 14933 |
| 3919 | CAAAGUCC A CCGCCUUC | 6185 | GAAGGCGG GGCTAGCTACAACGA GGACTTTG | 14934 |
| 3916 | AGUCCACC G CCUUCGCA | 6186 | TGCGAAGG GGCTAGCTACAACGA GGTGGACT | 14935 |
| 3910 | CCGCCUUC G CAACCCCC | 6187 | GGGGGTTG GGCTAGCTACAACGA GAAGGCGG | 14936 |
| 3907 | CCUUCGCA A CCCCCCGG | 6188 | CCGGGGGG GGCTAGCTACAACGA TGCGAAGG | 14937 |
| 3898 | CCCCCCGG G UGCACACA | 6189 | TGTGTGCA GGCTAGCTACAACGA CCGGGGGG | 14938 |
| 3896 | CCCCGGGU G CACACAGC | 6190 | GCTGTGTG GGCTAGCTACAACGA ACCCGGGG | 14939 |
| 3894 | CCGGGUGC A CACAGCAG | 6191 | CTGCTGTG GGCTAGCTACAACGA GCACCCGG | 14940 |
| 3892 | GGGUGCAC A CAGCAGCC | 6192 | GGCTGCTG GGCTAGCTACAACGA GTGCACCC | 14941 |
| 3889 | UGCACACA G CAGCCCGG | 6193 | CCGGGCTG GGCTAGCTACAACGA TGTGTGCA | 14942 |
| 3886 | ACACAGCA G CCCGGAAG | 6194 | CTTCCGGG GGCTAGCTACAACGA TGCTGTGT | 14943 |
| 3877 | CCCGGAAG A UGCCCACA | 6195 | TGTGGGCA GGCTAGCTACAACGA CTTCCGGG | 14944 |
| 3875 | CGGAAGAU G CCCACAAC | 6196 | GTTGTGGG GGCTAGCTACAACGA ATCTTCCG | 14945 |
| 3871 | AGAUGCCC A CAACGUGC | 6197 | GCACGTTG GGCTAGCTACAACGA GGGCATCT | 14946 |
| 3868 | UGCCCACA A CGUGCCCC | 6198 | GGGGCACG GGCTAGCTACAACGA TGTGGGCA | 14947 |
| 3866 | CCCACAAC G UGCCCCGA | 6199 | TCGGGGCA GGCTAGCTACAACGA GTTGTGGG | 14948 |
| 3864 | CACAACGU G CCCCGAAG | 6200 | CTTCGGGG GGCTAGCTACAACGA ACGTTGTG | 14949 |
| 3854 | CCCGAAGG G CAGAGCAG | 6201 | CTGCTCTG GGCTAGCTACAACGA CCTTCGGG | 14950 |
| 3849 | AGGGCAGA G CAGUGGAC | 6202 | GTCCACTG GGCTAGCTACAACGA TCTGCCCT | 14951 |
| 3846 | GCAGAGCA G UGGACCGC | 6203 | GCGGTCCA GGCTAGCTACAACGA TGCTCTGC | 14952 |
| 3842 | AGCAGUGG A CCGCCCGA | 6204 | TCGGGCGG GGCTAGCTACAACGA CCACTGCT | 14953 |
| 3839 | AGUGGACC G CCCGAGGA | 6205 | TCCTCGGG GGCTAGCTACAACGA GGTCCACT | 14954 |
| 3830 | CCCGAGGA G CCCUUCAA | 6206 | TTGAAGGG GGCTAGCTACAACGA TCCTCGGG | 14955 |
| 3821 | CCCUUCAA G UAGGAGAU | 6207 | ATCTCCTA GGCTAGCTACAACGA TTGAAGGG | 14956 |
| 3814 | AGUAGGAG A UGGGCCUG | 6208 | CAGGCCCA GGCTAGCTACAACGA CTCCTACT | 14957 |
| 3810 | GGAGAUGG G CCUGGGGG | 6209 | CCCCCAGG GGCTAGCTACAACGA CCATCTCC | 14958 |
| 3801 | CCUGGGGG A UAGUAAGC | 6210 | GCTTACTA GGCTAGCTACAACGA CCCCCAGG | 14959 |
| 3798 | GGGGGAUA G UAAGCUCC | 6211 | GGAGCTTA GGCTAGCTACAACGA TATCCCCC | 14960 |
| 3794 | GAUAGUAA G CUCCCCCU | 6212 | AGGGGGAG GGCTAGCTACAACGA TTACTATC | 14961 |
| 3785 | CUCCCCCU G CUGUCACC | 6213 | GGTGACAG GGCTAGCTACAACGA AGGGGGAG | 14962 |
| 3782 | CCCCUGCU G UCACCCCG | 6214 | CGGGGTGA GGCTAGCTACAACGA AGCAGGGG | 14963 |
| 3779 | CUGCUGUC A CCCCGCCG | 6215 | CGGCGGGG GGCTAGCTACAACGA GACAGCAG | 14964 |
| 3774 | GUCACCCC G CCGGCGCA | 6216 | TGCGCCGG GGCTAGCTACAACGA GGGGTGAC | 14965 |
| 3770 | CCCCGCCG G CGCACCGG | 6217 | CCGGTGCG GGCTAGCTACAACGA CGGCGGGG | 14966 |
| 3768 | CCGCCGGC G CACCGGAA | 6218 | TTCCGGTG GGCTAGCTACAACGA GCCGGCGG | 14967 |
| 3766 | GCCGGCGC A CCGGAAUG | 6219 | CATTCCGG GGCTAGCTACAACGA GCGCCGGC | 14968 |
| 3760 | GCACCGGA A UGACAUCA | 6220 | TGATGTCA GGCTAGCTACAACGA TCCGGTGC | 14969 |
| 3757 | CCGGAAUG A CAUCAGCG | 6221 | CGCTGATG GGCTAGCTACAACGA CATTCCGG | 14970 |
| 3755 | GGAAUGAC A UCAGCGUG | 6222 | CACGCTGA GGCTAGCTACAACGA GTCATTCC | 14971 |
| 3751 | UGACAUCA G CGUGUCUC | 6223 | GAGACACG GGCTAGCTACAACGA TGATGTCA | 14972 |
| 3749 | ACAUCAGC G UGUCUCGU | 6224 | ACGAGACA GGCTAGCTACAACGA GCTGATGT | 14973 |
| 3747 | AUCAGCGU G UCUCGUGA | 6225 | TCACGAGA GGCTAGCTACAACGA ACGCTGAT | 14974 |
| 3742 | CGUGUCUC G UGACCAAG | 6226 | CTTGGTCA GGCTAGCTACAACGA GAGACACG | 14975 |
| 3739 | GUCUCGUG A CCAAGUAA | 6227 | TTACTTGG GGCTAGCTACAACGA CACGAGAC | 14976 |
| 3734 | GUGACCAA G UAAAGGUC | 6228 | GACCTTTA GGCTAGCTACAACGA TTGGTCAC | 14977 |
| 3728 | AAGUAAAG G UCCGAGCC | 6229 | GGCTCGGA GGCTAGCTACAACGA CTTTACTT | 14978 |
| 3722 | AGGUCCGA G CCGCCGCA | 6230 | TGCGGCGG GGCTAGCTACAACGA TCGGACCT | 14979 |
| 3719 | UCCGAGCC G CCGCAGGU | 6231 | ACCTGCGG GGCTAGCTACAACGA GGCTCGGA | 14980 |
| 3716 | GAGCCGCC G CAGGUGCA | 6232 | TGCACCTG GGCTAGCTACAACGA GGCGGCTC | 14981 |
| 3712 | CGCCGCAG G UGCAUGGU | 6233 | ACCATGCA GGCTAGCTACAACGA CTGCGGCG | 14982 |
| 3710 | CCGCAGGU G CAUGGUGU | 6234 | ACACCATG GGCTAGCTACAACGA ACCTGCGG | 14983 |
| 3708 | GCAGGUGC A UGGUGUCA | 6235 | TGACACCA GGCTAGCTACAACGA GCACCTGC | 14984 |
| 3705 | GGUGCAUG G UGUCAAGG | 6236 | CCTTGACA GGCTAGCTACAACGA CATGCACC | 14985 |
| 3703 | UGCAUGGU G UCAAGGAC | 6237 | GTCCTTGA GGCTAGCTACAACGA ACCATGCA | 14986 |
| 3696 | UGUCAAGG A CCGCGCUC | 6238 | GAGCGCGG GGCTAGCTACAACGA CCTTGACA | 14987 |
| 3693 | CAAGGACC G CGCUCCGG | 6239 | CCGGAGCG GGCTAGCTACAACGA GGTCCTTG | 14988 |
| 3691 | AGGACCGC G CUCCGGGG | 6240 | CCCCGGAG GGCTAGCTACAACGA GCGGTCCT | 14989 |
| 3681 | UCCGGGGG G CGCCGGCC | 6241 | GGCCGGCG GGCTAGCTACAACGA CCCCCGGA | 14990 |
| 3679 | CGGGGGGC G CCGGCCAU | 6242 | ATGGCCGG GGCTAGCTACAACGA GCCCCCCG | 14991 |
| 3675 | GGGCGCCG G CCAUCCGA | 6243 | TCGGATGG GGCTAGCTACAACGA CGGCGCCC | 14992 |
| 3672 | CGCCGGCC A UCCGACGA | 6244 | TCGTCGGA GGCTAGCTACAACGA GGCCGGCG | 14993 |
| 3667 | GCCAUCCG A CGAGGUCC | 6245 | GGACCTCG GGCTAGCTACAACGA CGGATGGC | 14994 |
| 3662 | CCGACGAG G UCCUGGUC | 6246 | GACCAGGA GGCTAGCTACAACGA CTCGTCGG | 14995 |
| 3656 | AGGUCCUG G UCUACAUU | 6247 | AATGTAGA GGCTAGCTACAACGA CAGGACCT | 14996 |
| 3652 | CCUGGUCU A CAUUGGUG | 6248 | CACCAATG GGCTAGCTACAACGA AGACCAGG | 14997 |
| 3650 | UGGUCUAC A UUGGUGUA | 6249 | TACACCAA GGCTAGCTACAACGA GTAGACCA | 14998 |
| 3646 | CUACAUUG G UGUACAUU | 6250 | AATGTACA GGCTAGCTACAACGA CAATGTAG | 14999 |
| 3644 | ACAUUGGU G UACAUUUG | 6251 | CAAATGTA GGCTAGCTACAACGA ACCAATGT | 15000 |
| 3642 | AUUGGUGU A CAUUUGGG | 6252 | CCCAAATG GGCTAGCTACAACGA ACACCAAT | 15001 |
| 3640 | UGGUGUAC A UUUGGGUG | 6253 | CACCCAAA GGCTAGCTACAACGA GTACACCA | 15002 |
| 3634 | ACAUUUGG G UGAUUGGA | 6254 | TCCAATCA GGCTAGCTACAACGA CCAAATGT | 15003 |
| 3631 | UUUGGGUG A UUGGACCC | 6255 | GGGTCCAA GGCTAGCTACAACGA CACCCAAA | 15004 |
| 3626 | GUGAUUGG A CCCUUUGG | 6256 | CCAAAGGG GGCTAGCTACAACGA CCAATCAC | 15005 |
| 3617 | CCCUUUGG G CCGGCUAG | 6257 | CTAGCCGG GGCTAGCTACAACGA CCAAAGGG | 15006 |
| 3613 | UUGGGCCG G CUAGGGUC | 6258 | GACCCTAG GGCTAGCTACAACGA CGGCCCAA | 15007 |
| 3607 | CGGCUAGG G UCUUUGAG | 6259 | CTCAAAGA GGCTAGCTACAACGA CCTAGCCG | 15008 |
| 3599 | GUCUUUGA G CCGGCGCC | 6260 | GGCGCCGG GGCTAGCTACAACGA TCAAAGAC | 15009 |
| 3595 | UUGAGCCG G CGCCGUGG | 6261 | CCACGGCG GGCTAGCTACAACGA CGGCTCAA | 15010 |
| 3593 | GAGCCGGC G CCGUGGUA | 6262 | TACCACGG GGCTAGCTACAACGA GCCGGCTC | 15011 |
| 3590 | CCGGCGCC G UGGUAGAC | 6263 | GTCTACCA GGCTAGCTACAACGA GGCGCCGG | 15012 |
| 3587 | GCGCCGUG G UAGACAGU | 6264 | ACTGTCTA GGCTAGCTACAACGA CACGGCGC | 15013 |
| 3583 | CGUGGUAG A CAGUCCAG | 6265 | CTGGACTG GGCTAGCTACAACGA CTACCACG | 15014 |
| 3580 | GGUAGACA G UCCAGCAC | 6266 | GTGCTGGA GGCTAGCTACAACGA TGTCTACC | 15015 |
| 3575 | ACAGUCCA G CACACGCC | 6267 | GGCGTGTG GGCTAGCTACAACGA TGGACTGT | 15016 |
| 3573 | AGUCCAGC A CACGCCGU | 6268 | ACGGCGTG GGCTAGCTACAACGA GCTGGACT | 15017 |
| 3571 | UCCAGCAC A CGCCGUUG | 6269 | CAACGGCG GGCTAGCTACAACGA GTGCTGGA | 15018 |
| 3569 | CAGCACAC G CCGUUGAC | 6270 | GTCAACGG GGCTAGCTACAACGA GTGTGCTG | 15019 |
| 3566 | CACACGCC G UUGACGCA | 6271 | TGCGTCAA GGCTAGCTACAACGA GGCGTGTG | 15020 |
| 3562 | CGCCGUUG A CGCAGGUC | 6272 | GACCTGCG GGCTAGCTACAACGA CAACGGCG | 15021 |
| 3560 | CCGUUGAC G CAGGUCGC | 6273 | GCGACCTG GGCTAGCTACAACGA GTCAACGG | 15022 |
| 3556 | UGACGCAG G UCGCUAGG | 6274 | CCTAGCGA GGCTAGCTACAACGA CTGCGTCA | 15023 |
| 3553 | CGCAGGUC G CUAGGAAA | 6275 | TTTCCTAG GGCTAGCTACAACGA GACCTGCG | 15024 |
| 3543 | UAGGAAAG A CUGCGUCG | 6276 | CGACGCAG GGCTAGCTACAACGA CTTTCCTA | 15025 |
| 3540 | GAAAGACU G CGUCGCGG | 6277 | CCGCGACG GGCTAGCTACAACGA AGTCTTTC | 15026 |
| 3538 | AAGACUGC G UCGCGGUG | 6278 | CACCGCGA GGCTAGCTACAACGA GCAGTCTT | 15027 |
| 3535 | ACUGCGUC G CGGUGGAA | 6279 | TTCCACCG GGCTAGCTACAACGA GACGCAGT | 15028 |
| 3532 | GCGUCGCG G UGGAAACC | 6280 | GGTTTCCA GGCTAGCTACAACGA CGCGACGC | 15029 |
| 3526 | CGGUGGAA A CCACUUGA | 6281 | TCAAGTGG GGCTAGCTACAACGA TTCCACCG | 15030 |
| 3523 | UGGAAACC A CUUGAACU | 6282 | AGTTCAAG GGCTAGCTACAACGA GGTTTCCA | 15031 |
| 3517 | CCACUUGA A CUUCCCCC | 6283 | GGGGGAAG GGCTAGCTACAACGA TCAAGTGG | 15032 |
| 3505 | CCCCCUCG A CUUGGUUC | 6284 | GAACCAAG GGCTAGCTACAACGA CGAGGGGG | 15033 |
| 3500 | UCGACUUG G UUCUUGUC | 6285 | GACAAGAA GGCTAGCTACAACGA CAAGTCGA | 15034 |
| 3494 | UGGUUCUU G UCCCGGCC | 6286 | GGCCGGGA GGCTAGCTACAACGA AAGAACCA | 15035 |
| 3488 | UUGUCCCG G CCCGUGAG | 6287 | CTCACGGG GGCTAGCTACAACGA CGGGACAA | 15036 |
| 3484 | CCCGGCCC G UGAGGCUG | 6288 | CAGCCTCA GGCTAGCTACAACGA GGGCCGGG | 15037 |
| 3479 | CCCGUGAG G CUGGUGAU | 6289 | ATCACCAG GGCTAGCTACAACGA CTCACGGG | 15038 |
| 3475 | UGAGGCUG G UGAUAAUG | 6290 | CATTATCA GGCTAGCTACAACGA CAGCCTCA | 15039 |
| 3472 | GGCUGGUG A UAAUGCAG | 6291 | CTGCATTA GGCTAGCTACAACGA CACCAGCC | 15040 |
| 3469 | UGGUGAUA A UGCAGCCA | 6292 | TGGCTGCA GGCTAGCTACAACGA TATCACCA | 15041 |
| 3467 | GUGAUAAU G CAGCCAAA | 6293 | TTTGGCTG GGCTAGCTACAACGA ATTATCAC | 15042 |
| 3464 | AUAAUGCA G CCAAACAG | 6294 | CTGTTTGG GGCTAGCTACAACGA TGCATTAT | 15043 |
| 3459 | GCAGCCAA A CAGGCCCC | 6295 | GGGGCCTG GGCTAGCTACAACGA TTGGCTGC | 15044 |
| 3455 | CCAAACAG G CCCCGCGU | 6296 | ACGCGGGG GGCTAGCTACAACGA CTGTTTGG | 15045 |
| 3450 | CAGGCCCC G CGUCUGUU | 6297 | AACAGACG GGCTAGCTACAACGA GGGGCCTG | 15046 |
| 3448 | GGCCCCGC G UCUGUUGG | 6298 | CCAACAGA GGCTAGCTACAACGA GCGGGGCC | 15047 |
| 3444 | CCGCGUCU G UUGGGAGU | 6299 | ACTCCCAA GGCTAGCTACAACGA AGACGCGG | 15048 |
| 3437 | UGUUGGGA G UAGGCCGU | 6300 | ACGGCCTA GGCTAGCTACAACGA TCCCAACA | 15049 |
| 3433 | GGGAGUAG G CCGUAAUG | 6301 | CATTACGG GGCTAGCTACAACGA CTACTCCC | 15050 |
| 3430 | AGUAGGCC G UAAUGGGC | 6302 | GCCCATTA GGCTAGCTACAACGA GGCCTACT | 15051 |
| 3427 | AGGCCGUA A UGGGCGCG | 6303 | CGCGCCCA GGCTAGCTACAACGA TACGGCCT | 15052 |
| 3423 | CGUAAUGG G CGCGAGGA | 6304 | TCCTCGCG GGCTAGCTACAACGA CCATTACG | 15053 |
| 3421 | UAAUGGGC G CGAGGAGU | 6305 | ACTCCTCG GGCTAGCTACAACGA GCCCATTA | 15054 |
| 3414 | CGCGAGGA G UCGCCACC | 6306 | GGTGGCGA GGCTAGCTACAACGA TCCTCGCG | 15055 |
| 3411 | GAGGAGUC G CCACCCCU | 6307 | AGGGGTGG GGCTAGCTACAACGA GACTCCTC | 15056 |
| 3408 | GAGUCGCC A CCCCUGCC | 6308 | GGCAGGGG GGCTAGCTACAACGA GGCGACTC | 15057 |
| 3402 | CCACCCCU G CCCCUCAA | 6309 | TTGAGGGG GGCTAGCTACAACGA AGGGGTGG | 15058 |
| 3392 | CCCUCAAG A CUGUCGGC | 6310 | GCCGACAG GGCTAGCTACAACGA CTTGAGGG | 15059 |
| 3389 | UCAAGACU G UCGGCUGG | 6311 | CCAGCCGA GGCTAGCTACAACGA AGTCTTGA | 15060 |
| 3385 | GACUGUCG G CUGGUCCU | 6312 | AGGACCAG GGCTAGCTACAACGA CGACAGTC | 15061 |
| 3381 | GUCGGCUG G UCCUAGGA | 6313 | TCCTAGGA GGCTAGCTACAACGA CAGCCGAC | 15062 |
| 3372 | UCCUAGGA G UAUCUCCC | 6314 | GGGAGATA GGCTAGCTACAACGA TCCTAGGA | 15063 |
| 3370 | CUAGGAGU A UCUCCCUC | 6315 | GAGGGAGA GGCTAGCTACAACGA ACTCCTAG | 15064 |
| 3352 | CCCUUCGG G CGGAGACA | 6316 | TGTCTCCG GGCTAGCTACAACGA CCGAAGGG | 15065 |
| 3346 | GGGCGGAG A CAGGUAGA | 6317 | TCTACCTG GGCTAGCTACAACGA CTCCGCCC | 15066 |
| 3342 | GGAGACAG G UAGACCCA | 6318 | TGGGTCTA GGCTAGCTACAACGA CTGTCTCC | 15067 |
| 3338 | ACAGGUAG A CCCAUAAU | 6319 | ATTATGGG GGCTAGCTACAACGA CTACCTGT | 15068 |
| 3334 | GUAGACCC A UAAUGAUG | 6320 | CATCATTA GGCTAGCTACAACGA GGGTCTAC | 15069 |
| 3331 | GACCCAUA A UGAUGUCC | 6321 | GGACATCA GGCTAGCTACAACGA TATGGGTC | 15070 |
| 3328 | CCAUAAUG A UGUCCCCA | 6322 | TGGGGACA GGCTAGCTACAACGA CATTATGG | 15071 |
| 3326 | AUAAUGAU G UCCCCACA | 6323 | TGTGGGGA GGCTAGCTACAACGA ATCATTAT | 15072 |
| 3320 | AUGUCCCC A CACGCCGC | 6324 | GCGGCGTG GGCTAGCTACAACGA GGGGACAT | 15073 |
| 3318 | GUCCCCAC A CGCCGCGG | 6325 | CCGCGGCG GGCTAGCTACAACGA GTGGGGAC | 15074 |
| 3316 | CCCCACAC G CCGCGGUG | 6326 | CACCGCGG GGCTAGCTACAACGA GTGTGGGG | 15075 |
| 3313 | CACACGCC G CGGUGUCU | 6327 | AGACACCG GGCTAGCTACAACGA GGCGTGTG | 15076 |
| 3310 | ACGCCGCG G UGUCUCCC | 6328 | GGGAGACA GGCTAGCTACAACGA CGCGGCGT | 15077 |
| 3308 | GCCGCGGU G UCUCCCCC | 6329 | GGGGGAGA GGCTAGCTACAACGA ACCGCGGC | 15078 |
| 3295 | CCCCCCAG G UGAUGAUC | 6330 | GATCATCA GGCTAGCTACAACGA CTGGGGGG | 15079 |
| 3292 | CCCAGGUG A UGAUCUUG | 6331 | CAAGATCA GGCTAGCTACAACGA CACCTGGG | 15080 |
| 3289 | AGGUGAUG A UCUUGAUU | 6332 | AATCAAGA GGCTAGCTACAACGA CATCACCT | 15081 |
| 3283 | UGAUCUUG A UUUCCAUG | 6333 | CATGGAAA GGCTAGCTACAACGA CAAGATCA | 15082 |
| 3277 | UGAUUUCC A UGUCGGAG | 6334 | CTCCGACA GGCTAGCTACAACGA GGAAATCA | 15083 |
| 3275 | AUUUCCAU G UCGGAGAA | 6335 | TTCTCCGA GGCTAGCTACAACGA ATGGAAAT | 15084 |
| 3265 | CGGAGAAG A CGACGGGC | 6336 | GCCCGTCG GGCTAGCTACAACGA CTTCTCCG | 15085 |
| 3262 | AGAAGACG A CGGGCUCG | 6337 | CGAGCCCG GGCTAGCTACAACGA CGTCTTCT | 15086 |
| 3258 | GACGACGG G CUCGACCG | 6338 | CGGTCGAG GGCTAGCTACAACGA CCGTCGTC | 15087 |
| 3253 | CGGGCUCG A CCGCUACC | 6339 | GGTAGCGG GGCTAGCTACAACGA CGAGCCCG | 15088 |
| 3250 | GCUCGACC G CUACCGCC | 6340 | GGCGGTAG GGCTAGCTACAACGA GGTCGAGC | 15089 |
| 3247 | CGACCGCU A CCGCCAGG | 6341 | CCTGGCGG GGCTAGCTACAACGA AGCGGTCG | 15090 |
| 3244 | CCGCUACC G CCAGGUCU | 6342 | AGACCTGG GGCTAGCTACAACGA GGTAGCGG | 15091 |
| 3239 | ACCGCCAG G UCUCGUAG | 6343 | CTACGAGA GGCTAGCTACAACGA CTGGCGGT | 15092 |
| 3234 | CAGGUCUC G UAGACCUG | 6344 | CAGGTCTA GGCTAGCTACAACGA GAGACCTG | 15093 |
| 3230 | UCUCGUAG A CCUGUGUG | 6345 | CACACAGG GGCTAGCTACAACGA CTACGAGA | 15094 |
| 3226 | GUAGACCU G UGUGGGCC | 6346 | GGCCCACA GGCTAGCTACAACGA AGGTCTAC | 15095 |
| 3224 | AGACCUGU G UGGGCCCA | 6347 | TGGGCCCA GGCTAGCTACAACGA ACAGGTCT | 15096 |
| 3220 | CUGUGUGG G CCCAGUCC | 6348 | GGACTGGG GGCTAGCTACAACGA CCACACAG | 15097 |
| 3215 | UGGGCCCA G UCCUGCAG | 6349 | CTGCAGGA GGCTAGCTACAACGA TGGGCCCA | 15098 |
| 3210 | CCAGUCCU G CAGUGGAG | 6350 | CTCCACTG GGCTAGCTACAACGA AGGACTGG | 15099 |
| 3207 | GUCCUGCA G UGGAGUGA | 6351 | TCACTCCA GGCTAGCTACAACGA TGCAGGAC | 15100 |
| 3202 | GCAGUGGA G UGAGGUGG | 6352 | CCACCTCA GGCTAGCTACAACGA TCCACTGC | 15101 |
| 3197 | GGAGUGAG G UGGUCAUA | 6353 | TATGACCA GGCTAGCTACAACGA CTCACTCC | 15102 |
| 3194 | GUGAGGUG G UCAUAGAC | 6354 | GTCTATGA GGCTAGCTACAACGA CACCTCAC | 15103 |
| 3191 | AGGUGGUC A UAGACGGA | 6355 | TCCGTCTA GGCTAGCTACAACGA GACCACCT | 15104 |
| 3187 | GGUCAUAG A CGGACGUA | 6356 | TACGTCCG GGCTAGCTACAACGA CTATGACC | 15105 |
| 3183 | AUAGACGG A CGUACCUU | 6357 | AAGGTACG GGCTAGCTACAACGA CCGTCTAT | 15106 |
| 3181 | AGACGGAC G UACCUUUC | 6358 | GAAAGGTA GGCTAGCTACAACGA GTCCGTCT | 15107 |
| 3179 | ACGGACGU A CCUUUCAA | 6359 | TTGAAAGG GGCTAGCTACAACGA ACGTCCGT | 15108 |
| 3171 | ACCUUUCA A UUCGGCCA | 6360 | TGGCCGAA GGCTAGCTACAACGA TGAAAGGT | 15109 |
| 3166 | UCAAUUCG G CCAACUUC | 6361 | GAAGTTGG GGCTAGCTACAACGA CGAATTGA | 15110 |
| 3162 | UUCGGCCA A CUUCAUGA | 6362 | TCATGAAG GGCTAGCTACAACGA TGGCCGAA | 15111 |
| 3157 | CCAACUUC A UGAAGGCC | 6363 | GGCCTTCA GGCTAGCTACAACGA GAAGTTGG | 15112 |
| 3151 | UCAUGAAG G CCAUUUGG | 6364 | CCAAATGG GGCTAGCTACAACGA CTTCATGA | 15113 |
| 3148 | UGAAGGCC A UUUGGACA | 6365 | TGTCCAAA GGCTAGCTACAACGA GGCCTTCA | 15114 |
| 3142 | CCAUUUGG A CAUAUUGC | 6366 | GCAATATG GGCTAGCTACAACGA CCAAATGG | 15115 |
| 3140 | AUUUGGAC A UAUUGCCC | 6367 | GGGCAATA GGCTAGCTACAACGA GTCCAAAT | 15116 |
| 3138 | UUGGACAU A UUGCCCCC | 6368 | GGGGGCAA GGCTAGCTACAACGA ATGTCCAA | 15117 |
| 3135 | GACAUAUU G CCCCCCAC | 6369 | GTGGGGGG GGCTAGCTACAACGA AATATGTC | 15118 |
| 3128 | UGCCCCCC A CCGACUUU | 6370 | AAAGTCGG GGCTAGCTACAACGA GGGGGGCA | 15119 |
| 3124 | CCCCACCG A CUUUCCGC | 6371 | GCGGAAAG GGCTAGCTACAACGA CGGTGGGG | 15120 |
| 3117 | GACUUUCC G CACCAAAA | 6372 | TTTTGGTG GGCTAGCTACAACGA GGAAAGTC | 15121 |
| 3115 | CUUUCCGC A CCAAAAUG | 6373 | CATTTTGG GGCTAGCTACAACGA GCGGAAAG | 15122 |
| 3109 | GCACCAAA A UGCAUUCA | 6374 | TGAATGCA GGCTAGCTACAACGA TTTGGTGC | 15123 |
| 3107 | ACCAAAAU G CAUUCACG | 6375 | CGTGAATG GGCTAGCTACAACGA ATTTTGGT | 15124 |
| 3105 | CAAAAUGC A UUCACGGA | 6376 | TCCGTGAA GGCTAGCTACAACGA GCATTTTG | 15125 |
| 3101 | AUGCAUUC A CGGAUGAC | 6377 | GTCATCCG GGCTAGCTACAACGA GAATGCAT | 15126 |
| 3097 | AUUCACGG A UGACCCCU | 6378 | AGGGGTCA GGCTAGCTACAACGA CCGTGAAT | 15127 |
| 3094 | CACGGAUG A CCCCUUGA | 6379 | TCAAGGGG GGCTAGCTACAACGA CATCCGTG | 15128 |
| 3085 | CCCCUUGA G CCCGCACA | 6380 | TGTGCGGG GGCTAGCTACAACGA TCAAGGGG | 15129 |
| 3081 | UUGAGCCC G CACAAAGU | 6381 | ACTTTGTG GGCTAGCTACAACGA GGGCTCAA | 15130 |
| 3079 | GAGCCCGC A CAAAGUCC | 6382 | GGACTTTG GGCTAGCTACAACGA GCGGGCTC | 15131 |
| 3074 | CGCACAAA G UCCGGCAC | 6383 | GTGCCGGA GGCTAGCTACAACGA TTTGTGCG | 15132 |
| 3069 | AAAGUCCG G CACUUUUG | 6384 | CAAAAGTG GGCTAGCTACAACGA CGGACTTT | 15133 |
| 3067 | AGUCCGGC A CUUUUGCU | 6385 | AGCAAAAG GGCTAGCTACAACGA GCCGGACT | 15134 |
| 3061 | GCACUUUU G CUAUACCA | 6386 | TGGTATAG GGCTAGCTACAACGA AAAAGTGC | 15135 |
| 3058 | CUUUUGCU A UACCAGCC | 6387 | GGCTGGTA GGCTAGCTACAACGA AGCAAAAG | 15136 |
| 3056 | UUUGCUAU A CCAGCCUG | 6388 | CAGGCTGG GGCTAGCTACAACGA ATAGCAAA | 15137 |
| 3052 | CUAUACCA G CCUGGAGC | 6389 | GCTCCAGG GGCTAGCTACAACGA TGGTATAG | 15138 |
| 3045 | AGCCUGGA G CACCAUGA | 6390 | TCATGGTG GGCTAGCTACAACGA TCCAGGCT | 15139 |
| 3043 | CCUGGAGC A CCAUGAGC | 6391 | GCTCATGG GGCTAGCTACAACGA GCTCCAGG | 15140 |
| 3040 | GGAGCACC A UGAGCGGG | 6392 | CCCGCTCA GGCTAGCTACAACGA GGTGCTCC | 15141 |
| 3036 | CACCAUGA G CGGGCCGA | 6393 | TCGGCCCG GGCTAGCTACAACGA TCATGGTG | 15142 |
| 3032 | AUGAGCGG G CCGAGUAU | 6394 | ATACTCGG GGCTAGCTACAACGA CCGCTCAT | 15143 |
| 3027 | CGGGCCGA G UAUGGCGA | 6395 | TCGCCATA GGCTAGCTACAACGA TCGGCCCG | 15144 |
| 3025 | GGCCGAGU A UGGCGAGC | 6396 | GCTCGCCA GGCTAGCTACAACGA ACTCGGCC | 15145 |
| 3022 | CGAGUAUG G CGAGCAUA | 6397 | TATGCTCG GGCTAGCTACAACGA CATACTCG | 15146 |
| 3018 | UAUGGCGA G CAUAAUUU | 6398 | AAATTATG GGCTAGCTACAACGA TCGCCATA | 15147 |
| 3016 | UGGCGAGC A UAAUUUUG | 6399 | CAAAATTA GGCTAGCTACAACGA GCTCGCCA | 15148 |
| 3013 | CGAGCAUA A UUUUGGUG | 6400 | CACCAAAA GGCTAGCTACAACGA TATGCTCG | 15149 |
| 3007 | UAAUUUUG G UGAUGUCA | 6401 | TGACATCA GGCTAGCTACAACGA CAAAATTA | 15150 |
| 3004 | UUUUGGUG A UGUCAAAG | 6402 | CTTTGACA GGCTAGCTACAACGA CACCAAAA | 15151 |
| 3002 | UUGGUGAU G UCAAAGAU | 6403 | ATCTTTGA GGCTAGCTACAACGA ATCACCAA | 15152 |
| 2995 | UGUCAAAG A UUAGCUCU | 6404 | AGAGCTAA GGCTAGCTACAACGA CTTTGACA | 15153 |
| 2991 | AAAGAUUA G CUCUGGGU | 6405 | ACCCAGAG GGCTAGCTACAACGA TAATCTTT | 15154 |
| 2984 | AGCUCUGG G UGGACCAC | 6406 | GTGGTCCA GGCTAGCTACAACGA CCAGAGCT | 15155 |
| 2980 | CUGGGUGG A CCACACAC | 6407 | GTGTGTGG GGCTAGCTACAACGA CCACCCAG | 15156 |
| 2977 | GGUGGACC A CACACGUG | 6408 | CACGTGTG GGCTAGCTACAACGA GGTCCACC | 15157 |
| 2975 | UGGACCAC A CACGUGAG | 6409 | CTCACGTG GGCTAGCTACAACGA GTGGTCCA | 15158 |
| 2973 | GACCACAC A CGUGAGGA | 6410 | TCCTCACG GGCTAGCTACAACGA GTGTGGTC | 15159 |
| 2971 | CCACACAC G UGAGGAGA | 6411 | TCTCCTCA GGCTAGCTACAACGA GTGTGTGG | 15160 |
| 2962 | UGAGGAGA A UGAUGGCA | 6412 | TGCCATCA GGCTAGCTACAACGA TCTCCTCA | 15161 |
| 2959 | GGAGAAUG A UGGCACCG | 6413 | CGGTGCCA GGCTAGCTACAACGA CATTCTCC | 15162 |
| 2956 | GAAUGAUG G CACCGCGC | 6414 | GCGCGGTG GGCTAGCTACAACGA CATCATTC | 15163 |
| 2954 | AUGAUGGC A CCGCGCCC | 6415 | GGGCGCGG GGCTAGCTACAACGA GCCATCAT | 15164 |
| 2951 | AUGGCACC G CGCCCCCC | 6416 | GGGGGGCG GGCTAGCTACAACGA GGTGCCAT | 15165 |
| 2949 | GGCACCGC G CCCCCCCC | 6417 | GGGGGGGG GGCTAGCTACAACGA GCGGTGCC | 15166 |
| 2938 | CCCCCCGA A CGUUGAGG | 6418 | CCTCAACG GGCTAGCTACAACGA TCGGGGGG | 15167 |
| 2936 | CCCCGAAC G UUGAGGGG | 6419 | CCCCTCAA GGCTAGCTACAACGA GTTCGGGG | 15168 |
| 2923 | GGGGGGGG A UCCACACU | 6420 | AGTGTGGA GGCTAGCTACAACGA CCCCCCCC | 15169 |
| 2919 | GGGGAUCC A CACUUGCA | 6421 | TGCAAGTG GGCTAGCTACAACGA GGATCCCC | 15170 |
| 2917 | GGAUCCAC A CUUGCAAC | 6422 | GTTGCAAG GGCTAGCTACAACGA GTGGATCC | 15171 |
| 2913 | CCACACUU G CAACUGCG | 6423 | CGCAGTTG GGCTAGCTACAACGA AAGTGTGG | 15172 |
| 2910 | CACUUGCA A CUGCGCCU | 6424 | AGGCGCAG GGCTAGCTACAACGA TGCAAGTG | 15173 |
| 2907 | UUGCAACU G CGCCUCGG | 6425 | CCGAGGCG GGCTAGCTACAACGA AGTTGCAA | 15174 |
| 2905 | GCAACUGC G CCUCGGCU | 6426 | AGCCGAGG GGCTAGCTACAACGA GCAGTTGC | 15175 |
| 2899 | GCGCCUCG G CUCUGGUG | 6427 | CACCAGAG GGCTAGCTACAACGA CGAGGCGC | 15176 |
| 2893 | CGGCUCUG G UGAUAAGG | 6428 | CCTTATCA GGCTAGCTACAACGA CAGAGCCG | 15177 |
| 2890 | CUCUGGUG A UAAGGUAU | 6429 | ATACCTTA GGCTAGCTACAACGA CACCAGAG | 15178 |
| 2885 | GUGAUAAG G UAUUGCAA | 6430 | TTGCAATA GGCTAGCTACAACGA CTTATCAC | 15179 |
| 2883 | GAUAAGGU A UUGCAACC | 6431 | GGTTGCAA GGCTAGCTACAACGA ACCTTATC | 15180 |
| 2880 | AAGGUAUU G CAACCACC | 6432 | GGTGGTTG GGCTAGCTACAACGA AATACCTT | 15181 |
| 2877 | GUAUUGCA A CCACCAUA | 6433 | TATGGTGG GGCTAGCTACAACGA TGCAATAC | 15182 |
| 2874 | UUGCAACC A CCAUAUGA | 6434 | TCATATGG GGCTAGCTACAACGA GGTTGCAA | 15183 |
| 2871 | CAACCACC A UAUGAGCC | 6435 | GGCTCATA GGCTAGCTACAACGA GGTGGTTG | 15184 |
| 2869 | ACCACCAU A UGAGCCUA | 6436 | TAGGCTCA GGCTAGCTACAACGA ATGGTGGT | 15185 |
| 2865 | CCAUAUGA G CCUAGCGA | 6437 | TCGCTAGG GGCTAGCTACAACGA TCATATGG | 15186 |
| 2860 | UGAGCCUA G CGAGGAAC | 6438 | GTTCCTCG GGCTAGCTACAACGA TAGGCTCA | 15187 |
| 2853 | AGCGAGGA A CACUUUGU | 6439 | ACAAAGTG GGCTAGCTACAACGA TCCTCGCT | 15188 |
| 2851 | CGAGGAAC A CUUUGUAG | 6440 | CTACAAAG GGCTAGCTACAACGA GTTCCTCG | 15189 |
| 2846 | AACACUUU G UAGUAUGG | 6441 | CCATACTA GGCTAGCTACAACGA AAAGTGTT | 15190 |
| 2843 | ACUUUGUA G UAUGGUGA | 6442 | TCACCATA GGCTAGCTACAACGA TACAAAGT | 15191 |
| 2841 | UUUGUAGU A UGGUGACA | 6443 | TGTCACCA GGCTAGCTACAACGA ACTACAAA | 15192 |
| 2838 | GUAGUAUG G UGACAAGG | 6444 | CCTTGTCA GGCTAGCTACAACGA CATACTAC | 15193 |
| 2835 | GUAUGGUG A CAAGGUCA | 6445 | TGACCTTG GGCTAGCTACAACGA CACCATAC | 15194 |
| 2830 | GUGACAAG G UCAAGAGU | 6446 | ACTCTTGA GGCTAGCTACAACGA CTTGTCAC | 15195 |
| 2823 | GGUCAAGA G UGCUAGAC | 6447 | GTCTAGCA GGCTAGCTACAACGA TCTTGACC | 15196 |
| 2821 | UCAAGAGU G CUAGACCU | 6448 | AGGTCTAG GGCTAGCTACAACGA ACTCTTGA | 15197 |
| 2816 | AGUGCUAG A CCUACAAA | 6449 | TTTGTAGG GGCTAGCTACAACGA CTAGCACT | 15198 |
| 2812 | CUAGACCU A CAAAAACC | 6450 | GGTTTTTG GGCTAGCTACAACGA AGGTCTAG | 15199 |
| 2806 | CUACAAAA A CCACGCCU | 6451 | AGGCGTGG GGCTAGCTACAACGA TTTTGTAG | 15200 |
| 2803 | CAAAAACC A CGCCUCCG | 6452 | CGGAGGCG GGCTAGCTACAACGA GGTTTTTG | 15201 |
| 2801 | AAAACCAC G CCUCCGCA | 6453 | TGCGGAGG GGCTAGCTACAACGA GTGGTTTT | 15202 |
| 2795 | ACGCCUCC G CACGAUGC | 6454 | GCATCGTG GGCTAGCTACAACGA GGAGGCGT | 15203 |
| 2793 | GCCUCCGC A CGAUGCGG | 6455 | CCGCATCG GGCTAGCTACAACGA GCGGAGGC | 15204 |
| 2790 | UCCGCACG A UGCGGCCA | 6456 | TGGCCGCA GGCTAGCTACAACGA CGTGCGGA | 15205 |
| 2788 | CGCACGAU G CGGCCAUC | 6457 | GATGGCCG GGCTAGCTACAACGA ATCGTGCG | 15206 |
| 2785 | ACGAUGCG G CCAUCUCC | 6458 | GGAGATGG GGCTAGCTACAACGA CGCATCGT | 15207 |
| 2782 | AUGCGGCC A UCUCCCGG | 6459 | CCGGGAGA GGCTAGCTACAACGA GGCCGCAT | 15208 |
| 2774 | AUCUCCCG G UCCAUGGC | 6460 | GCCATGGA GGCTAGCTACAACGA CGGGAGAT | 15209 |
| 2770 | CCCGGUCC A UGGCGUAC | 6461 | GTACGCCA GGCTAGCTACAACGA GGACCGGG | 15210 |
| 2767 | GGUCCAUG G CGUACGCC | 6462 | GGCGTACG GGCTAGCTACAACGA CATGGACC | 15211 |
| 2765 | UCCAUGGC G UACGCCCG | 6463 | CGGGCGTA GGCTAGCTACAACGA GCCATGGA | 15212 |
| 2763 | CAUGGCGU A CGCCCGUG | 6464 | CACGGGCG GGCTAGCTACAACGA ACGCCATG | 15213 |
| 2761 | UGGCGUAC G CCCGUGGU | 6465 | ACCACGGG GGCTAGCTACAACGA GTACGCCA | 15214 |
| 2757 | GUACGCCC G UGGUGGUA | 6466 | TACCACCA GGCTAGCTACAACGA GGGCGTAC | 15215 |
| 2754 | CGCCCGUG G UGGUAACG | 6467 | CGTTACCA GGCTAGCTACAACGA CACGGGCG | 15216 |
| 2751 | CCGUGGUG G UAACGCCA | 6468 | TGGCGTTA GGCTAGCTACAACGA CACCACGG | 15217 |
| 2748 | UGGUGGUA A CGCCAGCA | 6469 | TGCTGGCG GGCTAGCTACAACGA TACCACCA | 15218 |
| 2746 | GUGGUAAC G CCAGCAGG | 6470 | CCTGCTGG GGCTAGCTACAACGA GTTACCAC | 15219 |
| 2742 | UAACGCCA G CAGGAGCA | 6471 | TGCTCCTG GGCTAGCTACAACGA TGGCGTTA | 15220 |
| 2736 | CAGCAGGA G CAGGAGUA | 6472 | TACTCCTG GGCTAGCTACAACGA TCCTGCTG | 15221 |
| 2730 | GAGCAGGA G UAGCGGCC | 6473 | GGCCGCTA GGCTAGCTACAACGA TCCTGCTC | 15222 |
| 2727 | CAGGAGUA G CGGCCAUA | 6474 | TATGGCCG GGCTAGCTACAACGA TACTCCTG | 15223 |
| 2724 | GAGUAGCG G CCAUACGC | 6475 | GCGTATGG GGCTAGCTACAACGA CGCTACTC | 15224 |
| 2721 | UAGCGGCC A UACGCCGU | 6476 | ACGGCGTA GGCTAGCTACAACGA GGCCGCTA | 15225 |
| 2719 | GCGGCCAU A CGCCGUAG | 6477 | CTACGGCG GGCTAGCTACAACGA ATGGCCGC | 15226 |
| 2717 | GGCCAUAC G CCGUAGAG | 6478 | CTCTACGG GGCTAGCTACAACGA GTATGGCC | 15227 |
| 2714 | CAUACGCC G UAGAGAGC | 6479 | GCTCTCTA GGCTAGCTACAACGA GGCGTATG | 15228 |
| 2707 | CGUAGAGA G CAUAUGCC | 6480 | GGCATATG GGCTAGCTACAACGA TCTCTACG | 15229 |
| 2705 | UAGAGAGC A UAUGCCGC | 6481 | GCGGCATA GGCTAGCTACAACGA GCTCTCTA | 15230 |
| 2703 | GAGAGCAU A UGCCGCCC | 6482 | GGGCGGCA GGCTAGCTACAACGA ATGCTCTC | 15231 |
| 2701 | GAGCAUAU G CCGCCCCA | 6483 | TGGGGCGG GGCTAGCTACAACGA ATATGCTC | 15232 |
| 2698 | CAUAUGCC G CCCCAGGG | 6484 | CCCTGGGG GGCTAGCTACAACGA GGCATATG | 15233 |
| 2689 | CCCCAGGG A CCAGCUUG | 6485 | CAAGCTGG GGCTAGCTACAACGA CCCTGGGG | 15234 |
| 2685 | AGGGACCA G CUUGCCUU | 6486 | AAGGCAAG GGCTAGCTACAACGA TGGTCCCT | 15235 |
| 2681 | ACCAGCUU G CCUUUGAU | 6487 | ATCAAAGG GGCTAGCTACAACGA AAGCTGGT | 15236 |
| 2674 | UGCCUUUG A UGUACCAG | 6488 | CTGGTACA GGCTAGCTACAACGA CAAAGGCA | 15237 |
| 2672 | CCUUUGAU G UACCAGGC | 6489 | GCCTGGTA GGCTAGCTACAACGA ATCAAAGG | 15238 |
| 2670 | UUUGAUGU A CCAGGCAG | 6490 | CTGCCTGG GGCTAGCTACAACGA ACATCAAA | 15239 |
| 2665 | UGUACCAG G CAGCACAG | 6491 | CTGTGCTG GGCTAGCTACAACGA CTGGTACA | 15240 |
| 2662 | ACCAGGCA G CACAGAAG | 6492 | CTTCTGTG GGCTAGCTACAACGA TGCCTGGT | 15241 |
| 2660 | CAGGCAGC A CAGAAGAA | 6493 | TTCTTCTG GGCTAGCTACAACGA GCTGCCTG | 15242 |
| 2652 | ACAGAAGA A CACGAGGA | 6494 | TCCTCGTG GGCTAGCTACAACGA TCTTCTGT | 15243 |
| 2650 | AGAAGAAC A CGAGGAAG | 6495 | CTTCCTCG GGCTAGCTACAACGA GTTCTTCT | 15244 |
| 2635 | AGGAGAGG A UGCCAUGC | 6496 | GCATGGCA GGCTAGCTACAACGA CCTCTCCT | 15245 |
| 2633 | GAGAGGAU G CCAUGCAC | 6497 | GTGCATGG GGCTAGCTACAACGA ATCCTCTC | 15246 |
| 2630 | AGGAUGCC A UGCACUCC | 6498 | GGAGTGCA GGCTAGCTACAACGA GGCATCCT | 15247 |
| 2628 | GAUGCCAU G CACUCCGG | 6499 | CCGGAGTG GGCTAGCTACAACGA ATGGCATC | 15248 |
| 2626 | UGCCAUGC A CUCCGGCC | 6500 | GGCCGGAG GGCTAGCTACAACGA GCATGGCA | 15249 |
| 2620 | GCACUCCG G CCAAGGAU | 6501 | ATCCTTGG GGCTAGCTACAACGA CGGAGTGC | 15250 |
| 2613 | GGCCAAGG A UGCUGCAU | 6502 | ATGCAGCA GGCTAGCTACAACGA CCTTGGCC | 15251 |
| 2611 | CCAAGGAU G CUGCAUUG | 6503 | CAATGCAG GGCTAGCTACAACGA ATCCTTGG | 15252 |
| 2608 | AGGAUGCU G CAUUGAGG | 6504 | CCTCAATG GGCTAGCTACAACGA AGCATCCT | 15253 |
| 2606 | GAUGCUGC A UUGAGGAC | 6505 | GTCCTCAA GGCTAGCTACAACGA GCAGCATC | 15254 |
| 2599 | CAUUGAGG A CCACCAGG | 6506 | CCTGGTGG GGCTAGCTACAACGA CCTCAATG | 15255 |
| 2596 | UGAGGACC A CCAGGUUC | 6507 | GAACCTGG GGCTAGCTACAACGA GGTCCTCA | 15256 |
| 2591 | ACCACCAG G UUCUCUAG | 6508 | CTAGAGAA GGCTAGCTACAACGA CTGGTGGT | 15257 |
| 2581 | UCUCUAGG G CAGCCUCG | 6509 | CGAGGCTG GGCTAGCTACAACGA CCTAGAGA | 15258 |
| 2578 | CUAGGGCA G CCUCGGCC | 6510 | GGCCGAGG GGCTAGCTACAACGA TGCCCTAG | 15259 |
| 2572 | CAGCCUCG G CCUGGGCU | 6511 | AGCCCAGG GGCTAGCTACAACGA CGAGGCTG | 15260 |
| 2566 | CGGCCUGG G CUACCAAC | 6512 | GTTGGTAG GGCTAGCTACAACGA CCAGGCCG | 15261 |
| 2563 | CCUGGGCU A CCAACAGC | 6513 | GCTGTTGG GGCTAGCTACAACGA AGCCCAGG | 15262 |
| 2559 | GGCUACCA A CAGCAUCA | 6514 | TGATGCTG GGCTAGCTACAACGA TGGTAGCC | 15263 |
| 2556 | UACCAACA G CAUCAUCC | 6515 | GGATGATG GGCTAGCTACAACGA TGTTGGTA | 15264 |
| 2554 | CCAACAGC A UCAUCCAC | 6516 | GTGGATGA GGCTAGCTACAACGA GCTGTTGG | 15265 |
| 2551 | ACAGCAUC A UCCACAAA | 6517 | TTTGTGGA GGCTAGCTACAACGA GATGCTGT | 15266 |
| 2547 | CAUCAUCC A CAAACAGG | 6518 | CCTGTTTG GGCTAGCTACAACGA GGATGATG | 15267 |
| 2543 | AUCCACAA A CAGGCACA | 6519 | TGTGCCTG GGCTAGCTACAACGA TTGTGGAT | 15268 |
| 2539 | ACAAACAG G CACAGACG | 6520 | CGTCTGTG GGCTAGCTACAACGA CTGTTTGT | 15269 |
| 2537 | AAACAGGC A CAGACGCG | 6521 | CGCGTCTG GGCTAGCTACAACGA GCCTGTTT | 15270 |
| 2533 | AGGCACAG A CGCGCGCG | 6522 | CGCGCGCG GGCTAGCTACAACGA CTGTGCCT | 15271 |
| 2531 | GCACAGAC G CGCGCGUC | 6523 | GACGCGCG GGCTAGCTACAACGA GTCTGTGC | 15272 |
| 2529 | ACAGACGC G CGCGUCUG | 6524 | CAGACGCG GGCTAGCTACAACGA GCGTCTGT | 15273 |
| 2527 | AGACGCGC G CGUCUGCC | 6525 | GGCAGACG GGCTAGCTACAACGA GCGCGTCT | 15274 |
| 2525 | ACGCGCGC G UCUGCCAG | 6526 | CTGGCAGA GGCTAGCTACAACGA GCGCGCGT | 15275 |
| 2521 | GCGCGUCU G CCAGGAGA | 6527 | TCTCCTGG GGCTAGCTACAACGA AGACGCGC | 15276 |
| 2505 | AAGGAAAA G CAACAGGA | 6528 | TCCTGTTG GGCTAGCTACAACGA TTTTCCTT | 15277 |
| 2502 | GAAAAGCA A CAGGACAU | 6529 | ATGTCCTG GGCTAGCTACAACGA TGCTTTTC | 15278 |
| 2497 | GCAACAGG A CAUACUCC | 6530 | GGAGTATG GGCTAGCTACAACGA CCTGTTGC | 15279 |
| 2495 | AACAGGAC A UACUCCCA | 6531 | TGGGAGTA GGCTAGCTACAACGA GTCCTGTT | 15280 |
| 2493 | CAGGACAU A CUCCCAUU | 6532 | AATGGGAG GGCTAGCTACAACGA ATGTCCTG | 15281 |
| 2487 | AUACUCCC A UUUGAUUG | 6533 | CAATCAAA GGCTAGCTACAACGA GGGAGTAT | 15282 |
| 2482 | CCCAUUUG A UUGCGAAG | 6534 | CTTCGCAA GGCTAGCTACAACGA CAAATGGG | 15283 |
| 2479 | AUUUGAUU G CGAAGGAG | 6535 | CTCCTTCG GGCTAGCTACAACGA AATCAAAT | 15284 |
| 2470 | CGAAGGAG A CAACCGCU | 6536 | AGCGGTTG GGCTAGCTACAACGA CTCCTTCC | 15285 |
| 2467 | AGGAGACA A CCGCUGAC | 6537 | GTCAGCGG GGCTAGCTACAACGA TGTCTCCT | 15286 |
| 2464 | AGACAACC G CUGACCCU | 6538 | AGGGTCAG GGCTAGCTACAACGA GGTTGTCT | 15287 |
| 2460 | AACCGCUG A CCCUACAC | 6539 | GTGTAGGG GGCTAGCTACAACGA CAGCGGTT | 15288 |
| 2455 | CUGACCCU A CACCGUAC | 6540 | GTACGGTG GGCTAGCTACAACGA AGGGTCAG | 15289 |
| 2453 | GACCCUAC A CCGUACAG | 6541 | CTGTACGG GGCTAGCTACAACGA GTAGGGTC | 15290 |
| 2450 | CCUACACC G UACAGGUA | 6542 | TACCTGTA GGCTAGCTACAACGA GGTGTAGG | 15291 |
| 2448 | UACACCGU A CAGGUAUU | 6543 | AATACCTG GGCTAGCTACAACGA ACGGTGTA | 15292 |
| 2444 | CCGUACAG G UAUUGCAC | 6544 | GTGCAATA GGCTAGCTACAACGA CTGTACGG | 15293 |
| 2442 | GUACAGGU A UUGCACGU | 6545 | ACGTGCAA GGCTAGCTACAACGA ACCTGTAC | 15294 |
| 2439 | CAGGUAUU G CACGUCCA | 6546 | TGGACGTG GGCTAGCTACAACGA AATACCTG | 15295 |
| 2437 | GGUAUUGC A CGUCCACG | 6547 | CGTGGACG GGCTAGCTACAACGA GCAATACC | 15296 |
| 2435 | UAUUGCAC G UCCACGAU | 6548 | ATCGTGGA GGCTAGCTACAACGA GTGCAATA | 15297 |
| 2431 | GCACGUCC A CGAUGUUC | 6549 | GAACATCG GGCTAGCTACAACGA GGACGTGC | 15298 |
| 2428 | CGUCCACG A UGUUCUGG | 6550 | CCAGAACA GGCTAGCTACAACGA CGTGGACG | 15299 |
| 2426 | UCCACGAU G UUCUGGUG | 6551 | CACCAGAA GGCTAGCTACAACGA ATCGTGGA | 15300 |
| 2420 | AUGUUCUG G UGGAGAUG | 6552 | CATCTCCA GGCTAGCTACAACGA CAGAACAT | 15301 |
| 2414 | UGGUGGAG A UGGAUCAA | 6553 | TTGATCCA GGCTAGCTACAACGA CTCCACCA | 15302 |
| 2410 | GGAGAUGG A UCAAACCA | 6554 | TGGTTTGA GGCTAGCTACAACGA CCATCTCC | 15303 |
| 2405 | UGGAUCAA A CCAGUGGA | 6555 | TCCACTGG GGCTAGCTACAACGA TTGATCCA | 15304 |
| 2401 | UCAAACCA G UGGACAGA | 6556 | TCTGTCCA GGCTAGCTACAACGA TGGTTTGA | 15305 |
| 2397 | ACCAGUGG A CAGAGCCG | 6557 | CGGCTCTG GGCTAGCTACAACGA CCACTGGT | 15306 |
| 2392 | UGGACAGA G CCGGUAGG | 6558 | CCTACCGG GGCTAGCTACAACGA TCTGTCCA | 15307 |
| 2388 | CAGAGCCG G UAGGGUGG | 6559 | CCACCCTA GGCTAGCTACAACGA CGGCTCTG | 15308 |
| 2383 | CCGGUAGG G UGGUGAAG | 6560 | CTTCACCA GGCTAGCTACAACGA CCTACCGG | 15309 |
| 2380 | GUAGGGUG G UGAAGGAG | 6561 | CTCCTTCA GGCTAGCTACAACGA CACCCTAC | 15310 |
| 2372 | GUGAAGGA G CAGGGCAG | 6562 | CTGCCCTG GGCTAGCTACAACGA TCCTTCAC | 15311 |
| 2367 | GGAGCAGG G CAGUAUUU | 6563 | AAATACTG GGCTAGCTACAACGA CCTGCTCC | 15312 |
| 2364 | GCAGGGCA G UAUUUGCC | 6564 | GGCAAATA GGCTAGCTACAACGA TGCCCTGC | 15313 |
| 2362 | AGGGCAGU A UUUGCCAC | 6565 | GTGGCAAA GGCTAGCTACAACGA ACTGCCCT | 15314 |
| 2358 | CAGUAUUU G CCACUCUG | 6566 | CAGAGTGG GGCTAGCTACAACGA AAATACTG | 15315 |
| 2355 | UAUUUGCC A CUCUGUAG | 6567 | CTACAGAG GGCTAGCTACAACGA GGCAAATA | 15316 |
| 2350 | GCCACUCU G UAGUGGAC | 6568 | GTCCACTA GGCTAGCTACAACGA AGAGTGGC | 15317 |
| 2347 | ACUCUGUA G UGGACAAC | 6569 | GTTGTCCA GGCTAGCTACAACGA TACAGAGT | 15318 |
| 2343 | UGUAGUGG A CAACAGCA | 6570 | TGCTGTTG GGCTAGCTACAACGA CCACTACA | 15319 |
| 2340 | AGUGGACA A CAGCAGCG | 6571 | CGCTGCTG GGCTAGCTACAACGA TGTCCACT | 15320 |
| 2337 | GGACAACA G CAGCGGGC | 6572 | GCCCGCTG GGCTAGCTACAACGA TGTTGTCC | 15321 |
| 2334 | CAACAGCA G CGGGCUGA | 6573 | TCAGCCCG GGCTAGCTACAACGA TGCTGTTG | 15322 |
| 2330 | AGCAGCGG G CUGAGCUC | 6574 | GAGCTCAG GGCTAGCTACAACGA CCGCTGCT | 15323 |
| 2325 | CGGGCUGA G CUCUGAUC | 6575 | GATCAGAG GGCTAGCTACAACGA TCAGCCCG | 15324 |
| 2319 | GAGCUCUG A UCUGUCCC | 6576 | GGGACAGA GGCTAGCTACAACGA CAGAGCTC | 15325 |
| 2315 | UCUGAUCU G UCCCUGUC | 6577 | GACAGGGA GGCTAGCTACAACGA AGATCAGA | 15326 |
| 2309 | CUGUCCCU G UCCUCCAA | 6578 | TTGGAGGA GGCTAGCTACAACGA AGGGACAG | 15327 |
| 2300 | UCCUCCAA A UCACAACG | 6579 | CGTTGTGA GGCTAGCTACAACGA TTGGAGGA | 15328 |
| 2297 | UCCAAAUC A CAACGCUC | 6580 | GAGCGTTG GGCTAGCTACAACGA GATTTGGA | 15329 |
| 2294 | AAAUCACA A CGCUCUCC | 6581 | GGAGAGCG GGCTAGCTACAACGA TGTGATTT | 15330 |
| 2292 | AUCACAAC G CUCUCCUC | 6582 | GAGGAGAG GGCTAGCTACAACGA GTTGTGAT | 15331 |
| 2281 | CUCCUCGA G UCCAAUUG | 6583 | CAATTGGA GGCTAGCTACAACGA TCGAGGAG | 15332 |
| 2276 | CGAGUCCA A UUGCAUGC | 6584 | GCATGCAA GGCTAGCTACAACGA TGGACTCG | 15333 |
| 2273 | GUCCAAUU G CAUGCGGC | 6585 | GCCGCATG GGCTAGCTACAACGA AATTGGAC | 15334 |
| 2271 | CCAAUUGC A UGCGGCGG | 6586 | CCGCCGCA GGCTAGCTACAACGA GCAATTGG | 15335 |
| 2269 | AAUUGCAU G CGGCGGUG | 6587 | CACCGCCG GGCTAGCTACAACGA ATGCAATT | 15336 |
| 2266 | UGCAUGCG G CGGUGAGC | 6588 | GCTCACCG GGCTAGCTACAACGA CGCATGCA | 15337 |
| 2263 | AUGCGGCG G UGAGCCUG | 6589 | CAGGCTCA GGCTAGCTACAACGA CGCCGCAT | 15338 |
| 2259 | GGCGGUGA G CCUGUGCU | 6590 | AGCACAGG GGCTAGCTACAACGA TCACCGCC | 15339 |
| 2255 | GUGAGCCU G UGCUCCAC | 6591 | GTGGAGCA GGCTAGCTACAACGA AGGCTCAC | 15340 |
| 2253 | GAGCCUGU G CUCCACGC | 6592 | GCGTGGAG GGCTAGCTACAACGA ACAGGCTC | 15341 |
| 2248 | UGUGCUCC A CGCCCCCC | 6593 | GGGGGGCG GGCTAGCTACAACGA GGAGCACA | 15342 |
| 2246 | UGCUCCAC G CCCCCCAC | 6594 | GTGGGGGG GGCTAGCTACAACGA GTGGAGCA | 15343 |
| 2239 | CGCCCCCC A CAUACAUC | 6595 | GATGTATG GGCTAGCTACAACGA GGGGGGCG | 15344 |
| 2237 | CCCCCCAC A UACAUCCU | 6596 | AGGATGTA GGCTAGCTACAACGA GTGGGGGG | 15345 |
| 2235 | CCCCACAU A CAUCCUAA | 6597 | TTAGGATG GGCTAGCTACAACGA ATGTGGGG | 15346 |
| 2233 | CCACAUAC A UCCUAACC | 6598 | GGTTAGGA GGCTAGCTACAACGA GTATGTGG | 15347 |
| 2227 | ACAUCCUA A CCUUAAAG | 6599 | CTTTAAGG GGCTAGCTACAACGA TAGGATGT | 15348 |
| 2218 | CCUUAAAG A UGGAAAAA | 6600 | TTTTTCCA GGCTAGCTACAACGA CTTTAAGG | 15349 |
| 2210 | AUGGAAAA A UUGACAGU | 6601 | ACTGTCAA GGCTAGCTACAACGA TTTTCCAT | 15350 |
| 2206 | AAAAAUUG A CAGUGCAG | 6602 | CTGCACTG GGCTAGCTACAACGA CAATTTTT | 15351 |
| 2203 | AAUUGACA G UGCAGGGG | 6603 | CCCCTGCA GGCTAGCTACAACGA TGTCAATT | 15352 |
| 2201 | UUGACAGU G CAGGGGUA | 6604 | TACCCCTG GGCTAGCTACAACGA ACTGTCAA | 15353 |
| 2195 | GUGCAGGG G UAGUGCCA | 6605 | TGGCACTA GGCTAGCTACAACGA CCCTGCAC | 15354 |
| 2192 | CAGGGGUA G UGCCAAAG | 6606 | CTTTGGCA GGCTAGCTACAACGA TACCCCTG | 15355 |
| 2190 | GGGGUAGU G CCAAAGCC | 6607 | GGCTTTGG GGCTAGCTACAACGA ACTACCCC | 15356 |
| 2184 | GUGCCAAA G CCUGUAUG | 6608 | CATACAGG GGCTAGCTACAACGA TTTGGCAC | 15357 |
| 2180 | CAAAGCCU G UAUGGGUA | 6609 | TACCCATA GGCTAGCTACAACGA AGGCTTTG | 15358 |
| 2178 | AAGCCUGU A UGGGUAGU | 6610 | ACTACCCA GGCTAGCTACAACGA ACAGGCTT | 15359 |
| 2174 | CUGUAUGG G UAGUCAAC | 6611 | GTTGACTA GGCTAGCTACAACGA CCATACAG | 15360 |
| 2171 | UAUGGGUA G UCAACUAU | 6612 | ATAGTTGA GGCTAGCTACAACGA TACCCATA | 15361 |
| 2167 | GGUAGUCA A CUAUGCAU | 6613 | ATGCATAG GGCTAGCTACAACGA TGACTACC | 15362 |
| 2164 | AGUCAACU A UGCAUCUA | 6614 | TAGATGCA GGCTAGCTACAACGA AGTTGACT | 15363 |
| 2162 | UCAACUAU G CAUCUAGG | 6615 | CCTAGATG GGCTAGCTACAACGA ATAGTTGA | 15364 |
| 2160 | AACUAUGC A UCUAGGUG | 6616 | CACCTAGA GGCTAGCTACAACGA GCATAGTT | 15365 |
| 2154 | GCAUCUAG G UGUUAACC | 6617 | GGTTAACA GGCTAGCTACAACGA CTAGATGC | 15366 |
| 2152 | AUCUAGGU G UUAACCAA | 6618 | TTGGTTAA GGCTAGCTACAACGA ACCTAGAT | 15367 |
| 2148 | AGGUGUUA A CCAAGGCC | 6619 | GGCCTTGG GGCTAGCTACAACGA TAACACCT | 15368 |
| 2142 | UAACCAAG G CCCCGAAC | 6620 | GTTCGGGG GGCTAGCTACAACGA CTTGGTTA | 15369 |
| 2135 | GGCCCCGA A CCGCACUU | 6621 | AAGTGCGG GGCTAGCTACAACGA TCGGGGCC | 15370 |
| 2132 | CCCGAACC G CACUUUGC | 6622 | GCAAAGTG GGCTAGCTACAACGA GGTTCGGG | 15371 |
| 2130 | CGAACCGC A CUUUGCGU | 6623 | ACGCAAAG GGCTAGCTACAACGA GCGGTTCG | 15372 |
| 2125 | CGCACUUU G CGUAAGUG | 6624 | CACTTACG GGCTAGCTACAACGA AAAGTGCG | 15373 |
| 2123 | CACUUUGC G UAAGUGGC | 6625 | GCCACTTA GGCTAGCTACAACGA GCAAAGTG | 15374 |
| 2119 | UUGCGUAA G UGGCCUCG | 6626 | CGAGGCCA GGCTAGCTACAACGA TTACGCAA | 15375 |
| 2116 | CGUAAGUG G CCUCGGGG | 6627 | CCCCGAGG GGCTAGCTACAACGA CACTTACG | 15376 |
| 2108 | GCCUCGGG G UGCUUCCG | 6628 | CGGAAGCA GGCTAGCTACAACGA CCCGAGGC | 15377 |
| 2106 | CUCGGGGU G CUUCCGGA | 6629 | TCCGGAAG GGCTAGCTACAACGA ACCCCGAG | 15378 |
| 2096 | UUCCGGAA G CAGUCCGU | 6630 | ACGGACTG GGCTAGCTACAACGA TTCCGGAA | 15379 |
| 2093 | CGGAAGCA G UCCGUGGG | 6631 | CCCACGGA GGCTAGCTACAACGA TGCTTCCG | 15380 |
| 2089 | AGCAGUCC G UGGGGCAG | 6632 | CTGCCCCA GGCTAGCTACAACGA GGACTGCT | 15381 |
| 2084 | UCCGUGGG G CAGGUUAA | 6633 | TTAACCTG GGCTAGCTACAACGA CCCACGGA | 15382 |
| 2080 | UGGGGCAG G UUAAGGUG | 6634 | CACCTTAA GGCTAGCTACAACGA CTGCCCCA | 15383 |
| 2074 | AGGUUAAG G UGUCGUUA | 6635 | TAACGACA GGCTAGCTACAACGA CTTAACCT | 15384 |
| 2072 | GUUAAGGU G UCGUUACC | 6636 | GGTAACGA GGCTAGCTACAACGA ACCTTAAC | 15385 |
| 2069 | AAGGUGUC G UUACCGGC | 6637 | GCCGGTAA GGCTAGCTACAACGA GACACCTT | 15386 |
| 2066 | GUGUCGUU A CCGGCCCC | 6638 | GGGGCCGG GGCTAGCTACAACGA AACGACAC | 15387 |
| 2062 | CGUUACCG G CCCCCCCG | 6639 | CGGGGGGG GGCTAGCTACAACGA CGGTAACG | 15388 |
| 2053 | CCCCCCCG A UGUUGCAC | 6640 | GTGCAACA GGCTAGCTACAACGA CGGGGGGG | 15389 |
| 2051 | CCCCCGAU G UUGCACGG | 6641 | CCGTGCAA GGCTAGCTACAACGA ATCGGGGG | 15390 |
| 2048 | CCGAUGUU G CACGGGGG | 6642 | CCCCCGTG GGCTAGCTACAACGA AACATCGG | 15391 |
| 2046 | GAUGUUGC A CGGGGGGC | 6643 | GCCCCCCG GGCTAGCTACAACGA GCAACATC | 15392 |
| 2039 | CACGGGGG G CCCCCGCA | 6644 | TGCGGGGG GGCTAGCTACAACGA CCCCCGTG | 15393 |
| 2033 | GGGCCCCC G CACGUCUU | 6645 | AAGACGTG GGCTAGCTACAACGA GGGGGCCC | 15394 |
| 2031 | GCCCCCGC A CGUCUUGG | 6646 | CCAAGACG GGCTAGCTACAACGA GCGGGGGC | 15395 |
| 2029 | CCCCGCAC G UCUUGGUG | 6647 | CACCAAGA GGCTAGCTACAACGA GTGCGGGG | 15396 |
| 2023 | ACGUCUUG G UGAACCCA | 6648 | TGGGTTCA GGCTAGCTACAACGA CAAGACGT | 15397 |
| 2019 | CUUGGUGA A CCCAGUGC | 6649 | GCACTGGG GGCTAGCTACAACGA TCACCAAG | 15398 |
| 2014 | UGAACCCA G UGCCAUUC | 6650 | GAATGGCA GGCTAGCTACAACGA TGGGTTCA | 15399 |
| 2012 | AACCCAGU G CCAUUCAU | 6651 | ATGAATGG GGCTAGCTACAACGA ACTGGGTT | 15400 |
| 2009 | CCAGUGCC A UUCAUCCA | 6652 | TGGATGAA GGCTAGCTACAACGA GGCACTGG | 15401 |
| 2005 | UGCCAUUC A UCCAUGUG | 6653 | CACATGGA GGCTAGCTACAACGA GAATGGCA | 15402 |
| 2001 | AUUCAUCC A UGUGCAGC | 6654 | GCTGCACA GGCTAGCTACAACGA GGATGAAT | 15403 |
| 1999 | UCAUCCAU G UGCAGCCG | 6655 | CGGCTGCA GGCTAGCTACAACGA ATGGATGA | 15404 |
| 1997 | AUCCAUGU G CAGCCGAA | 6656 | TTCGGCTG GGCTAGCTACAACGA ACATGGAT | 15405 |
| 1994 | CAUGUGCA G CCGAACCA | 6657 | TGGTTCGG GGCTAGCTACAACGA TGCACATG | 15406 |
| 1989 | GCAGCCGA A CCAGUUGC | 6658 | GCAACTGG GGCTAGCTACAACGA TCGGCTGC | 15407 |
| 1985 | CCGAACCA G UUGCCUUG | 6659 | CAAGGCAA GGCTAGCTACAACGA TGGTTCGG | 15408 |
| 1982 | AACCAGUU G CCUUGCGG | 6660 | CCGCAAGG GGCTAGCTACAACGA AACTGGTT | 15409 |
| 1977 | GUUGCCUU G CGGCGGCC | 6661 | GGCCGCCG GGCTAGCTACAACGA AAGGCAAC | 15410 |
| 1974 | GCCUUGCG G CGGCCGCG | 6662 | CGCGGCCG GGCTAGCTACAACGA CGCAAGGC | 15411 |
| 1971 | UUGCGGCG G CCGCGUGU | 6663 | ACACGCGG GGCTAGCTACAACGA CGCCGCAA | 15412 |
| 1968 | CGGCGGCC G CGUGUUGU | 6664 | ACAACACG GGCTAGCTACAACGA GGCCGCCG | 15413 |
| 1966 | GCGGCCGC G UGUUGUUG | 6665 | CAACAACA GGCTAGCTACAACGA GCGGCCGC | 15414 |
| 1964 | GGCCGCGU G UUGUUGAG | 6666 | CTCAACAA GGCTAGCTACAACGA ACGCGGCC | 15415 |
| 1961 | CGCGUGUU G UUGAGGAG | 6667 | CTCCTCAA GGCTAGCTACAACGA AACACGCG | 15416 |
| 1953 | GUUGAGGA G CAGCACGU | 6668 | ACGTGCTG GGCTAGCTACAACGA TCCTCAAC | 15417 |
| 1950 | GAGGAGCA G CACGUCCG | 6669 | CGGACGTG GGCTAGCTACAACGA TGCTCCTC | 15418 |
| 1948 | GGAGCAGC A CGUCCGUC | 6670 | GACGGACG GGCTAGCTACAACGA GCTGCTCC | 15419 |
| 1946 | AGCAGCAC G UCCGUCUC | 6671 | GAGACGGA GGCTAGCTACAACGA GTGCTGCT | 15420 |
| 1942 | GCACGUCC G UCUCGUUC | 6672 | GAACGAGA GGCTAGCTACAACGA GGACGTGC | 15421 |
| 1937 | UCCGUCUC G UUCGCCCC | 6673 | GGGGCGAA GGCTAGCTACAACGA GAGACGGA | 15422 |
| 1933 | UCUCGUUC G CCCCCCAG | 6674 | CTGGGGGG GGCTAGCTACAACGA GAACGAGA | 15423 |
| 1925 | GCCCCCCA G UUAUACGU | 6675 | ACGTATAA GGCTAGCTACAACGA TGGGGGGC | 15424 |
| 1922 | CCCCAGUU A UACGUGGG | 6676 | CCCACGTA GGCTAGCTACAACGA AACTGGGG | 15425 |
| 1920 | CCAGUUAU A CGUGGGGG | 6677 | CCCCCACG GGCTAGCTACAACGA ATAACTGG | 15426 |
| 1918 | AGUUAUAC G UGGGGGCG | 6678 | CGCCCCCA GGCTAGCTACAACGA GTATAACT | 15427 |
| 1912 | ACGUGGGG G CGCCGAAA | 6679 | TTTCGGCG GGCTAGCTACAACGA CCCCACGT | 15428 |
| 1910 | GUGGGGGC G CCGAAACG | 6680 | CGTTTCGG GGCTAGCTACAACGA GCCCCCAC | 15429 |
| 1904 | GCGCCGAA A CGGUCGGU | 6681 | ACCGACCG GGCTAGCTACAACGA TTCGGCGC | 15430 |
| 1901 | CCGAAACG G UCGGUCGU | 6682 | ACGACCGA GGCTAGCTACAACGA CGTTTCGG | 15431 |
| 1897 | AACGGUCG G UCGUCCCC | 6683 | GGGGACGA GGCTAGCTACAACGA CGACCGTT | 15432 |
| 1894 | GGUCGGUC G UCCCCACC | 6684 | GGTGGGGA GGCTAGCTACAACGA GACCGACC | 15433 |
| 1888 | UCGUCCCC A CCACAACA | 6685 | TGTTGTGG GGCTAGCTACAACGA GGGGACGA | 15434 |
| 1885 | UCCCCACC A CAACAGGG | 6686 | CCCTGTTG GGCTAGCTACAACGA GGTGGGGA | 15435 |
| 1882 | CCACCACA A CAGGGCUU | 6687 | AAGCCCTG GGCTAGCTACAACGA TGTGGTGG | 15436 |
| 1877 | ACAACAGG G CUUGGGGU | 6688 | ACCCCAAG GGCTAGCTACAACGA CCTGTTGT | 15437 |
| 1870 | GGCUUGGG G UGAAGCAA | 6689 | TTGCTTCA GGCTAGCTACAACGA CCCAAGCC | 15438 |
| 1865 | GGGGUGAA G CAAUACAC | 6690 | GTGTATTG GGCTAGCTACAACGA TTCACCCC | 15439 |
| 1862 | GUGAAGCA A UACACUGG | 6691 | CCAGTGTA GGCTAGCTACAACGA TGCTTCAC | 15440 |
| 1860 | GAAGCAAU A CACUGGAC | 6692 | GTCCAGTG GGCTAGCTACAACGA ATTGCTTC | 15441 |
| 1858 | AGCAAUAC A CUGGACCA | 6693 | TGGTCCAG GGCTAGCTACAACGA GTATTGCT | 15442 |
| 1853 | UACACUGG A CCACAUAC | 6694 | GTATGTGG GGCTAGCTACAACGA CCAGTGTA | 15443 |
| 1850 | ACUGGACC A CAUACCUG | 6695 | CAGGTATG GGCTAGCTACAACGA GGTCCAGT | 15444 |
| 1848 | UGGACCAC A UACCUGCG | 6696 | CGCAGGTA GGCTAGCTACAACGA GTGGTCCA | 15445 |
| 1846 | GACCACAU A CCUGCGAU | 6697 | ATCGCAGG GGCTAGCTACAACGA ATGTGGTC | 15446 |
| 1842 | ACAUACCU G CGAUGCGG | 6698 | CCGCATCG GGCTAGCTACAACGA AGGTATGT | 15447 |
| 1839 | UACCUGCG A UGCGGGUA | 6699 | TACCCGCA GGCTAGCTACAACGA CGCAGGTA | 15448 |
| 1837 | CCUGCGAU G CGGGUACG | 6700 | CGTACCCG GGCTAGCTACAACGA ATCGCAGG | 15449 |
| 1833 | CGAUGCGG G UACGAUAC | 6701 | GTATCGTA GGCTAGCTACAACGA CCGCATCG | 15450 |
| 1831 | AUGCGGGU A CGAUACCA | 6702 | TGGTATCG GGCTAGCTACAACGA ACCCGCAT | 15451 |
| 1828 | CGGGUACG A UACCACAC | 6703 | GTGTGGTA GGCTAGCTACAACGA CGTACCCG | 15452 |
| 1826 | GGUACGAU A CCACACGG | 6704 | CCGTGTGG GGCTAGCTACAACGA ATCGTACC | 15453 |
| 1823 | ACGAUACC A CACGGCCG | 6705 | CGGCCGTG GGCTAGCTACAACGA GGTATCGT | 15454 |
| 1821 | GAUACCAC A CGGCCGCG | 6706 | CGCGGCCG GGCTAGCTACAACGA GTGGTATC | 15455 |
| 1818 | ACCACACG G CCGCGGUG | 6707 | CACCGCGG GGCTAGCTACAACGA CGTGTGGT | 15456 |
| 1815 | ACACGGCC G CGGUGCGU | 6708 | ACGCACCG GGCTAGCTACAACGA GGCCGTGT | 15457 |
| 1812 | CGGCCGCG G UGCGUAGU | 6709 | ACTACGCA GGCTAGCTACAACGA CGCGGCCG | 15458 |
| 1810 | GCCGCGGU G CGUAGUGC | 6710 | GCACTACG GGCTAGCTACAACGA ACCGCGGC | 15459 |
| 1808 | CGCGGUGC G UAGUGCCA | 6711 | TGGCACTA GGCTAGCTACAACGA GCACCGCG | 15460 |
| 1805 | GGUGCGUA G UGCCAGCA | 6712 | TGCTGGCA GGCTAGCTACAACGA TACGCACC | 15461 |
| 1803 | UGCGUAGU G CCAGCAAU | 6713 | ATTGCTGG GGCTAGCTACAACGA ACTACGCA | 15462 |
| 1799 | UAGUGCCA G CAAUAGGG | 6714 | CCCTATTG GGCTAGCTACAACGA TGGCACTA | 15463 |
| 1796 | UGCCAGCA A UAGGGCCU | 6715 | AGGCCCTA GGCTAGCTACAACGA TGCTGGCA | 15464 |
| 1791 | GCAAUAGG G CCUCUGGU | 6716 | ACCAGAGG GGCTAGCTACAACGA CCTATTGC | 15465 |
| 1784 | GGCCUCUG G UCCGAGUU | 6717 | AACTCGGA GGCTAGCTACAACGA CAGAGGCC | 15466 |
| 1778 | UGGUCCGA G UUGUGGCC | 6718 | GGCCACAA GGCTAGCTACAACGA TCGGACCA | 15467 |
| 1775 | UCCGAGUU G UGGCCCUC | 6719 | GAGGGCCA GGCTAGCTACAACGA AACTCGGA | 15468 |
| 1772 | GAGUUGUG G CCCUCGGU | 6720 | ACCGAGGG GGCTAGCTACAACGA CACAACTC | 15469 |
| 1765 | GGCCCUCG G UGUAGGUG | 6721 | CACCTACA GGCTAGCTACAACGA CGAGGGCC | 15470 |
| 1763 | CCCUCGGU G UAGGUGAU | 6722 | ATCACCTA GGCTAGCTACAACGA ACCGAGGG | 15471 |
| 1759 | CGGUGUAG G UGAUAGGA | 6723 | TCCTATCA GGCTAGCTACAACGA CTACACCG | 15472 |
| 1756 | UGUAGGUG A UAGGACCC | 6724 | GGGTCCTA GGCTAGCTACAACGA CACCTACA | 15473 |
| 1751 | GUGAUAGG A CCCCACCC | 6725 | GGGTGGGG GGCTAGCTACAACGA CCTATCAC | 15474 |
| 1746 | AGGACCCC A CCCCUGAG | 6726 | CTCAGGGG GGCTAGCTACAACGA GGGGTCCT | 15475 |
| 1738 | ACCCCUGA G CGAACUUG | 6727 | CAAGTTCG GGCTAGCTACAACGA TCAGGGGT | 15476 |
| 1734 | CUGAGCGA A CUUGUCAA | 6728 | TTGACAAG GGCTAGCTACAACGA TCGCTCAG | 15477 |
| 1730 | GCGAACUU G UCAAUGGA | 6729 | TCCATTGA GGCTAGCTACAACGA AAGTTCGC | 15478 |
| 1726 | ACUUGUCA A UGGAGCGG | 6730 | CCGCTCCA GGCTAGCTACAACGA TGACAAGT | 15479 |
| 1721 | UCAAUGGA G CGGCAGCU | 6731 | AGCTGCCG GGCTAGCTACAACGA TCCATTGA | 15480 |
| 1718 | AUGGAGCG G CAGCUGGC | 6732 | GCCAGCTG GGCTAGCTACAACGA CGCTCCAT | 15481 |
| 1715 | GAGCGGCA G CUGGCCAA | 6733 | TTGGCCAG GGCTAGCTACAACGA TGCCGCTC | 15482 |
| 1711 | GGCAGCUG G CCAAGCGC | 6734 | GCGCTTGG GGCTAGCTACAACGA CAGCTGCC | 15483 |
| 1706 | CUGGCCAA G CGCUGUGG | 6735 | CCACAGCG GGCTAGCTACAACGA TTGGCCAG | 15484 |
| 1704 | GGCCAAGC G CUGUGGGC | 6736 | GCCCACAG GGCTAGCTACAACGA GCTTGGCC | 15485 |
| 1701 | CAAGCGCU G UGGGCAUC | 6737 | GATGCCCA GGCTAGCTACAACGA AGCGCTTG | 15486 |
| 1697 | CGCUGUGG G CAUCCGGA | 6738 | TCCGGATG GGCTAGCTACAACGA CCACAGCG | 15487 |
| 1695 | CUGUGGGC A UCCGGACG | 6739 | CGTCCGGA GGCTAGCTACAACGA GCCCACAG | 15488 |
| 1689 | GCAUCCGG A CGAGUUGA | 6740 | TCAACTCG GGCTAGCTACAACGA CCGGATGC | 15489 |
| 1685 | CCGGACGA G UUGAACCU | 6741 | AGGTTCAA GGCTAGCTACAACGA TCGTCCGG | 15490 |
| 1680 | CGAGUUGA A CCUGUGUG | 6742 | CACACAGG GGCTAGCTACAACGA TCAACTCG | 15491 |
| 1676 | UUGAACCU G UGUGCAUA | 6743 | TATGCACA GGCTAGCTACAACGA AGGTTCAA | 15492 |
| 1674 | GAACCUGU G UGCAUAGA | 6744 | TCTATGCA GGCTAGCTACAACGA ACAGGTTC | 15493 |
| 1672 | ACCUGUGU G CAUAGAAC | 6745 | GTTCTATG GGCTAGCTACAACGA ACACAGGT | 15494 |
| 1670 | CUGUGUGC A UAGAACAG | 6746 | CTGTTCTA GGCTAGCTACAACGA GCACACAG | 15495 |
| 1665 | UGCAUAGA A CAGUGCAG | 6747 | CTGCACTG GGCTAGCTACAACGA TCTATGCA | 15496 |
| 1662 | AUAGAACA G UGCAGCAA | 6748 | TTGCTGCA GGCTAGCTACAACGA TGTTCTAT | 15497 |
| 1660 | AGAACAGU G CAGCAAUG | 6749 | CATTGCTG GGCTAGCTACAACGA ACTGTTCT | 15498 |
| 1657 | ACAGUGCA G CAAUGAAC | 6750 | GTTCATTG GGCTAGCTACAACGA TGCACTGT | 15499 |
| 1654 | GUGCAGCA A UGAACCCG | 6751 | CGGGTTCA GGCTAGCTACAACGA TGCTGCAC | 15500 |
| 1650 | AGCAAUGA A CCCGGUUU | 6752 | AAACCGGG GGCTAGCTACAACGA TCATTGCT | 15501 |
| 1645 | UGAACCCG G UUUGGAGG | 6753 | CCTCCAAA GGCTAGCTACAACGA CGGGTTCA | 15502 |
| 1634 | UGGAGGGA G UCAUUGCA | 6754 | TGCAATGA GGCTAGCTACAACGA TCCCTCCA | 15503 |
| 1631 | AGGGAGUC A UUGCAGUU | 6755 | AACTGCAA GGCTAGCTACAACGA GACTCCCT | 15504 |
| 1628 | GAGUCAUU G CAGUUCAG | 6756 | CTGAACTG GGCTAGCTACAACGA AATGACTC | 15505 |
| 1625 | UCAUUGCA G UUCAGGGC | 6757 | GCCCTGAA GGCTAGCTACAACGA TGCAATGA | 15506 |
| 1618 | AGUUCAGG G CAGUCCUG | 6758 | CAGGACTG GGCTAGCTACAACGA CCTGAACT | 15507 |
| 1615 | UCAGGGCA G UCCUGUUA | 6759 | TAACAGGA GGCTAGCTACAACGA TGCCCTGA | 15508 |
| 1610 | GCAGUCCU G UUAAUGUG | 6760 | CACATTAA GGCTAGCTACAACGA AGGACTGC | 15509 |
| 1606 | UCCUGUUA A UGUGCCAG | 6761 | CTGGCACA GGCTAGCTACAACGA TAACAGGA | 15510 |
| 1604 | CUGUUAAU G UGCCAGCU | 6762 | AGCTGGCA GGCTAGCTACAACGA ATTAACAG | 15511 |
| 1602 | GUUAAUGU G CCAGCUGC | 6763 | GCAGCTGG GGCTAGCTACAACGA ACATTAAC | 15512 |
| 1598 | AUGUGCCA G CUGCCGUU | 6764 | AACGGCAG GGCTAGCTACAACGA TGGCACAT | 15513 |
| 1595 | UGCCAGCU G CCGUUGGU | 6765 | ACCAACGG GGCTAGCTACAACGA AGCTGGCA | 15514 |
| 1592 | CAGCUGCC G UUGGUGUU | 6766 | AACACCAA GGCTAGCTACAACGA GGCAGCTG | 15515 |
| 1588 | UGCCGUUG G UGUUAAUA | 6767 | TATTAACA GGCTAGCTACAACGA CAACGGCA | 15516 |
| 1586 | CCGUUGGU G UUAAUAAG | 6768 | CTTATTAA GGCTAGCTACAACGA ACCAACGG | 15517 |
| 1582 | UGGUGUUA A UAAGCUGG | 6769 | CCAGCTTA GGCTAGCTACAACGA TAACACCA | 15518 |
| 1578 | GUUAAUAA G CUGGAUAU | 6770 | ATATCCAG GGCTAGCTACAACGA TTATTAAC | 15519 |
| 1573 | UAAGCUGG A UAUUCUGA | 6771 | TCAGAATA GGCTAGCTACAACGA CCAGCTTA | 15520 |
| 1571 | AGCUGGAU A UUCUGAGA | 6772 | TCTCAGAA GGCTAGCTACAACGA ATCCAGCT | 15521 |
| 1563 | AUUCUGAG A UGCUCCAG | 6773 | CTGGAGCA GGCTAGCTACAACGA CTCAGAAT | 15522 |
| 1561 | UCUGAGAU G CUCCAGAU | 6774 | ATCTGGAG GGCTAGCTACAACGA ATCTCAGA | 15523 |
| 1554 | UGCUCCAG A UGUAAAGA | 6775 | TCTTTACA GGCTAGCTACAACGA CTGGAGCA | 15524 |
| 1552 | CUCCAGAU G UAAAGAGG | 6776 | CCTCTTTA GGCTAGCTACAACGA ATCTGGAG | 15525 |
| 1542 | AAAGAGGG A UGCCACCC | 6777 | GGGTGGCA GGCTAGCTACAACGA CCCTCTTT | 15526 |
| 1540 | ACACCCAU G GGAGGGUA | 6778 | TAGGGTGG GGCTAGCTACAACGA ATCCCTCT | 15527 |
| 1537 | GGGAUGCC A CCCUACUA | 6779 | TAGTAGGG GGCTAGCTACAACGA GGCATCCC | 15528 |
| 1532 | GCCACCCU A CUAGUGGU | 6780 | ACCACTAG GGCTAGCTACAACGA AGGGTGGC | 15529 |
| 1528 | CCCUACUA G UGGUGUGG | 6781 | CCACACCA GGCTAGCTACAACGA TAGTAGGG | 15530 |
| 1525 | UACUAGUG G UGUGGCCC | 6782 | GGGCCACA GGCTAGCTACAACGA CACTAGTA | 15531 |
| 1523 | CUAGUGGU G UGGCCCUG | 6783 | CAGGGCCA GGCTAGCTACAACGA ACCACTAG | 15532 |
| 1520 | GUGGUGUG G CCCUGCGC | 6784 | GCGCAGGG GGCTAGCTACAACGA CACACCAC | 15533 |
| 1515 | GUGGCCCU G CGCCCCCC | 6785 | GGGGGGCG GGCTAGCTACAACGA AGGGCCAC | 15534 |
| 1513 | GGCCCUGC G CCCCCCCU | 6786 | AGGGGGGG GGCTAGCTACAACGA GCAGGGCC | 15535 |
| 1504 | CCCCCCCU G UCGUGUAG | 6787 | CTACACGA GGCTAGCTACAACGA AGGGGGGG | 15536 |
| 1501 | CCCCUGUC G UGUAGGUG | 6788 | CACCTACA GGCTAGCTACAACGA GACAGGGG | 15537 |
| 1499 | CCUGUCGU G UAGGUGUC | 6789 | GACACCTA GGCTAGCTACAACGA ACGACAGG | 15538 |
| 1495 | UCGUGUAG G UGUCCCCG | 6790 | CGGGGACA GGCTAGCTACAACGA CTACACGA | 15539 |
| 1493 | GUGUAGGU G UCCCCGUC | 6791 | GACGGGGA GGCTAGCTACAACGA ACCTACAC | 15540 |
| 1487 | GUGUCCCC G UCAACGCC | 6792 | GGCGTTGA GGCTAGCTACAACGA GGGGACAC | 15541 |
| 1483 | CCCCGUCA A CGCCGGCA | 6793 | TGCCGGCG GGCTAGCTACAACGA TGACGGGG | 15542 |
| 1481 | CCGUCAAC G CCGGCAAA | 6794 | TTTGCCGG GGCTAGCTACAACGA GTTGACGG | 15543 |
| 1477 | CAACGCCG G CAAAGAGU | 6795 | ACTCTTTG GGCTAGCTACAACGA CGGCGTTG | 15544 |
| 1470 | GGCAAAGA G UAGCAUCA | 6796 | TGATGCTA GGCTAGCTACAACGA TCTTTGCC | 15545 |
| 1467 | AAAGAGUA G CAUCACAA | 6797 | TTGTGATG GGCTAGCTACAACGA TACTCTTT | 15546 |
| 1465 | AGAGUAGC A UCACAAUG | 6798 | GATTGTGA GGCTAGCTACAACGA GCTACTCT | 15547 |
| 1462 | GUAGCAUC A CAAUCAAC | 6799 | GTTGATTG GGCTAGCTACAACGA GATGCTAC | 15548 |
| 1459 | GCAUCACA A UCAACACC | 6800 | GGTGTTGA GGCTAGCTACAACGA TGTGATGC | 15549 |
| 1455 | CACAAUCA A CACCUUAG | 6801 | CTAAGGTG GGCTAGCTACAACGA TGATTGTG | 15550 |
| 1453 | CAAUCAAC A CCUUAGCC | 6802 | GGCTAAGG GGCTAGCTACAACGA GTTGATTG | 15551 |
| 1447 | ACACCUUA G CCCAGUUC | 6803 | GAACTGGG GGCTAGCTACAACGA TAAGGTGT | 15552 |
| 1442 | UUAGCCCA G UUCCCCAC | 6804 | GTGGGGAA GGCTAGCTACAACGA TGGGCTAA | 15553 |
| 1435 | AGUUCCCC A CCAUGGAA | 6805 | TTCCATGG GGCTAGCTACAACGA GGGGAACT | 15554 |
| 1432 | UCCCCACC A UGGAAUAA | 6806 | TTATTCCA GGCTAGCTACAACGA GGTGGGGA | 15555 |
| 1427 | ACCAUGGA A UAAUAGGC | 6807 | GCCTATTA GGCTAGCTACAACGA TCCATGGT | 15556 |
| 1424 | AUGGAAUA A UAGGCAAG | 6808 | CTTGCCTA GGCTAGCTACAACGA TATTCCAT | 15557 |
| 1420 | AAUAAUAG G CAAGGCCC | 6809 | GGGCCTTG GGCTAGCTACAACGA CTATTATT | 15558 |
| 1415 | UAGGCAAG G CCCGCCAG | 6810 | CTGGCGGG GGCTAGCTACAACGA CTTGCCTA | 15559 |
| 1411 | CAAGGCCC G CCAGGACU | 6811 | AGTCCTGG GGCTAGCTACAACGA GGGCCTTG | 15560 |
| 1405 | CCGCCAGG A CUCCCCAG | 6812 | CTGGGGAG GGCTAGCTACAACGA CCTGGCGG | 15561 |
| 1397 | ACUCCCCA G UGGGCCCC | 6813 | GGGGCCCA GGCTAGCTACAACGA TGGGGAGT | 15562 |
| 1393 | CCCAGUGG G CCCCCGCC | 6814 | GGCGGGGG GGCTAGCTACAACGA CCACTGGG | 15563 |
| 1387 | GGGCCCCC G CCACCAUG | 6815 | CATGGTGG GGCTAGCTACAACGA GGGGGCCC | 15564 |
| 1384 | CCCCCGCC A CCAUGUCC | 6816 | GGACATGG GGCTAGCTACAACGA GGCGGGGG | 15565 |
| 1381 | CCGCCACC A UGUCCACG | 6817 | CGTGGACA GGCTAGCTACAACGA GGTGGCGG | 15566 |
| 1379 | GCCACCAU G UCCACGAC | 6818 | GTCGTGGA GGCTAGCTACAACGA ATGGTGGC | 15567 |
| 1375 | CCAUGUCC A CGACGGCU | 6819 | AGCCGTCG GGCTAGCTACAACGA GGACATGG | 15568 |
| 1372 | UGUCCACG A CGGCUUGU | 6820 | ACAAGCCG GGCTAGCTACAACGA CGTGGACA | 15569 |
| 1369 | CCACGACG G CUUGUGGG | 6821 | CCCACAAG GGCTAGCTACAACGA CGTCGTGG | 15570 |
| 1365 | GACGGCUU G UGGGAUCC | 6822 | GGATCCCA GGCTAGCTACAACGA AAGCCGTC | 15571 |
| 1360 | CUUGUGGG A UCCGGAGC | 6823 | GCTCCGGA GGCTAGCTACAACGA CCCACAAG | 15572 |
| 1353 | GAUCCGGA G CAACUGCG | 6824 | CGCAGTTG GGCTAGCTACAACGA TCCGGATC | 15573 |
| 1350 | CCGGAGCA A CUGCGAUA | 6825 | TATCGCAG GGCTAGCTACAACGA TGCTCCGG | 15574 |
| 1347 | GAGCAACU G CGAUACCA | 6826 | TGGTATCG GGCTAGCTACAACGA AGTTGCTC | 15575 |
| 1344 | CAACUGCG A UACCACUA | 6827 | TAGTGGTA GGCTAGCTACAACGA CGCAGTTG | 15576 |
| 1342 | ACUGCGAU A CCACUAGG | 6828 | CCTAGTGG GGCTAGCTACAACGA ATCGCAGT | 15577 |
| 1339 | GCGAUACC A CUAGGGCU | 6829 | AGCCCTAG GGCTAGCTACAACGA GGTATCGC | 15578 |
| 1333 | CCACUAGG G CUGUUGUA | 6830 | TACAACAG GGCTAGCTACAACGA CCTAGTGG | 15579 |
| 1330 | CUAGGGCU G UUGUAGGU | 6831 | ACCTACAA GGCTAGCTACAACGA AGCCCTAG | 15580 |
| 1327 | GGGCUGUU G UAGGUGAC | 6832 | GTCACCTA GGCTAGCTACAACGA AACAGCCC | 15581 |
| 1323 | UGUUGUAG G UGACCAAU | 6833 | ATTGGTCA GGCTAGCTACAACGA CTACAACA | 15582 |
| 1320 | UGUAGGUG A CCAAUUCA | 6834 | TCAATTGG GGCTAGCTACAACGA CACCTACA | 15583 |
| 1316 | GGUGACCA A UUCAUCAU | 6835 | ATGATGAA GGCTAGCTACAACGA TGGTCACC | 15584 |
| 1312 | ACCAAUUC A UCAUCAUA | 6836 | TATGATGA GGCTAGCTACAACGA GAATTGGT | 15585 |
| 1309 | AAUUCAUC A UCAUAUCC | 6837 | GGATATGA GGCTAGCTACAACGA GATGAATT | 15586 |
| 1306 | UCAUCAUC A UAUCCCAA | 6838 | TTGGGATA GGCTAGCTACAACGA GATGATGA | 15587 |
| 1304 | AUCAUCAU A UCCCAAGC | 6839 | GCTTGGGA GGCTAGCTACAACGA ATGATGAT | 15588 |
| 1297 | UAUCCCAA G CCAUGCGA | 6840 | TCGCATGG GGCTAGCTACAACGA TTGGGATA | 15589 |
| 1294 | CCCAAGCC A UGCGAUGG | 6841 | CCATCGCA GGCTAGCTACAACGA GGCTTGGG | 15590 |
| 1292 | CAAGCCAU G CGAUGGCC | 6842 | GGCCATCG GGCTAGCTACAACGA ATGGCTTG | 15591 |
| 1289 | GCCAUGCG A UGGCCUGA | 6843 | TCAGGCCA GGCTAGCTACAACGA CGCATGGC | 15592 |
| 1286 | AUGCGAUG G CCUGAUAC | 6844 | GTATCAGG GGCTAGCTACAACGA CATCGCAT | 15593 |
| 1281 | AUGGCCUG A UACGUGGC | 6845 | GCCACGTA GGCTAGCTACAACGA CAGGCCAT | 15594 |
| 1279 | GGCCUGAU A CGUGGCCG | 6846 | CGGCCACG GGCTAGCTACAACGA ATCAGGCC | 15595 |
| 1277 | CCUGAUAC G UGGCCGGG | 6847 | CCCGGCCA GGCTAGCTACAACGA GTATCAGG | 15596 |
| 1274 | GAUACGUG G CCGGGAUA | 6848 | TATCCCGG GGCTAGCTACAACGA CACGTATC | 15597 |
| 1268 | UGGCCGGG A UAGAUCGA | 6849 | TCGATCTA GGCTAGCTACAACGA CCCGGCCA | 15598 |
| 1264 | CGGGAUAG A UCGAGCAA | 6850 | TTGCTCGA GGCTAGCTACAACGA CTATCCCG | 15599 |
| 1259 | UAGAUCGA G CAAUUACA | 6851 | TGTAATTG GGCTAGCTACAACGA TCGATCTA | 15600 |
| 1256 | AUCGAGCA A UUACAGUC | 6852 | GACTGTAA GGCTAGCTACAACGA TGCTCGAT | 15601 |
| 1253 | GAGCAAUU A CAGUCCUG | 6853 | CAGGACTG GGCTAGCTACAACGA AATTGCTC | 15602 |
| 1250 | CAAUUACA G UCCUGUAC | 6854 | GTACAGGA GGCTAGCTACAACGA TGTAATTG | 15603 |
| 1245 | ACAGUCCU G UACUGUCU | 6855 | AGACAGTA GGCTAGCTACAACGA AGGACTGT | 15604 |
| 1243 | AGUCCUGU A CUGUCUCA | 6856 | TGAGACAG GGCTAGCTACAACGA ACAGGACT | 15605 |
| 1240 | CCUGUACU G UCUCAUAC | 6857 | GTATGAGA GGCTAGCTACAACGA AGTACAGG | 15606 |
| 1235 | ACUGUCUC A UACCGGCG | 6858 | CGCCGGTA GGCTAGCTACAACGA GAGACAGT | 15607 |
| 1233 | UGUCUCAU A CCGGCGAG | 6859 | CTCGCCGG GGCTAGCTACAACGA ATGAGACA | 15608 |
| 1229 | UCAUACCG G CGAGGCGA | 6860 | TCGCCTCG GGCTAGCTACAACGA CGGTATGA | 15609 |
| 1224 | CCGGCGAG G CGAGAAGG | 6861 | CCTTCTCG GGCTAGCTACAACGA CTCGCCGG | 15610 |
| 1216 | GCGAGAAG G UGAACAGC | 6862 | GCTGTTCA GGCTAGCTACAACGA CTTCTCGC | 15611 |
| 1212 | GAAGGUGA A CAGCUGAG | 6863 | CTCAGCTG GGCTAGCTACAACGA TCACCTTC | 15612 |
| 1209 | GGUGAACA G CUGAGAGA | 6864 | TCTCTCAG GGCTAGCTACAACGA TGTTCACC | 15613 |
| 1201 | GCUGAGAG A CGAGGAAG | 6865 | CTTCCTCG GGCTAGCTACAACGA CTCTCAGC | 15614 |
| 1192 | CGAGGAAG A CAGAUCCG | 6866 | CGGATCTG GGCTAGCTACAACGA CTTCCTCG | 15615 |
| 1188 | GAAGACAG A UCCGCAGA | 6867 | TCTGCGGA GGCTAGCTACAACGA CTGTCTTC | 15616 |
| 1184 | ACAGAUCC G CAGAGAUC | 6868 | GATCTCTG GGCTAGCTACAACGA GGATCTGT | 15617 |
| 1178 | CCGCAGAG A UCCCCCAC | 6869 | GTGGGGGA GGCTAGCTACAACGA CTCTGCGG | 15618 |
| 1171 | GAUCCCCC A CGUACAUA | 6870 | TATGTACC GGCTAGCTACAACGA GGGGGATC | 15619 |
| 1169 | UCCCCCAC G UACAUAGC | 6871 | GCTATGTA GGCTAGCTACAACGA GTGGGGGA | 15620 |
| 1167 | CCCCACGU A CAUAGCAG | 6872 | CTGCTATG GGCTAGCTACAACGA ACGTGGGG | 15621 |
| 1165 | CCACGUAC A UAGCAGAG | 6873 | CTCTGCTA GGCTAGCTACAACGA GTACGTGG | 15622 |
| 1162 | CGUACAUA G CAGAGCAG | 6874 | CTGCTCTG GGCTAGCTACAACGA TATGTACG | 15623 |
| 1157 | AUAGCAGA G CAGAAAGC | 6875 | GCTTTCTG GGCTAGCTACAACGA TCTGCTAT | 15624 |
| 1150 | AGCAGAAA G CAGCCGCC | 6876 | GGCGGCTG GGCTAGCTACAACGA TTTCTGCT | 15625 |
| 1147 | AGAAAGCA G CCGCCCCA | 6877 | TGGGGCGG GGCTAGCTACAACGA TGCTTTCT | 15626 |
| 1144 | AAGCAGCC G CCCCAACG | 6878 | CGTTGGGG GGCTAGCTACAACGA GGCTGCTT | 15627 |
| 1138 | CCGCCCCA A CGAGCAAA | 6879 | TTTGCTCG GGCTAGCTACAACGA TGGGGCGG | 15628 |
| 1134 | CCCAACGA G CAAAUCGA | 6880 | TCGATTTG GGCTAGCTACAACGA TCGTTGGG | 15629 |
| 1130 | ACGAGCAA A UCGACGUG | 6881 | CACGTCGA GGCTAGCTACAACGA TTGCTCGT | 15630 |
| 1126 | GCAAAUCG A CGUGACGC | 6882 | GCGTCACG GGCTAGCTACAACGA CGATTTGC | 15631 |
| 1124 | AAAUCGAC G UGACGCCG | 6883 | CGGCGTCA GGCTAGCTACAACGA GTCGATTT | 15632 |
| 1121 | UCGACGUG A CGCCGUAU | 6884 | ATACGGCG GGCTAGCTACAACGA CACGTCGA | 15633 |
| 1119 | GACGUGAC G CCGUAUCG | 6885 | CGATACGG GGCTAGCTACAACGA GTCACGTC | 15634 |
| 1116 | GUGACGCC G UAUCGUCG | 6886 | CGACGATA GGCTAGCTACAACGA GGCGTCAC | 15635 |
| 1114 | GACGCCGU A UCGUCGUA | 6887 | TACGACGA GGCTAGCTACAACGA ACGGCGTC | 15636 |
| 1111 | GCCGUAUC G UCGUAGUG | 6888 | CACTACGA GGCTAGCTACAACGA GATACGGC | 15637 |
| 1108 | GUAUCGUC G UAGUGGGG | 6889 | CCCCACTA GGCTAGCTACAACGA GACGATAC | 15638 |
| 1105 | UCGUCGUA G UGGGGAUG | 6890 | CATCCCCA GGCTAGCTACAACGA TACGACGA | 15639 |
| 1099 | UAGUGGGG A UGCUGGCA | 6891 | TGCCAGCA GGCTAGCTACAACGA CCCCACTA | 15640 |
| 1097 | GUGGGGAU G CUGGCAUU | 6892 | AATGCCAG GGCTAGCTACAACGA ATCCCCAC | 15641 |
| 1093 | GGAUGCUG G CAUUCCUG | 6893 | CAGGAATG GGCTAGCTACAACGA CAGCATCC | 15642 |
| 1091 | AUGCUGGC A UUCCUGGC | 6894 | GCCAGGAA GGCTAGCTACAACGA GCCAGCAT | 15643 |
| 1084 | CAUUCCUG G CCGCGAGC | 6895 | GCTCGCGG GGCTAGCTACAACGA CAGGAATG | 15644 |
| 1081 | UCCUGGCC G CGAGCGUG | 6896 | CACGCTCG GGCTAGCTACAACGA GGCCAGGA | 15645 |
| 1077 | GGCCGCGA G CGUGGGAG | 6897 | CTCCCACG GGCTAGCTACAACGA TCGCGGCC | 15646 |
| 1075 | CCGCGAGC G UGGGAGUG | 6898 | CACTCCCA GGCTAGCTACAACGA GCTCGCGG | 15647 |
| 1069 | GCGUGGGA G UGAGCGCU | 6899 | AGCGCTCA GGCTAGCTACAACGA TCCCACGC | 15648 |
| 1065 | GGGAGUGA G CGCUACCC | 6900 | GGGTAGCG GGCTAGCTACAACGA TCACTCCC | 15649 |
| 1063 | GAGUGAGC G CUACCCAG | 6901 | CTGGGTAG GGCTAGCTACAACGA GCTCACTC | 15650 |
| 1060 | UGAGCGCU A CCCAGCAG | 6902 | CTGCTGGG GGCTAGCTACAACGA AGCGCTCA | 15651 |
| 1055 | GCUACCCA G CAGCGGGA | 6903 | TCCCGCTG GGCTAGCTACAACGA TGGGTAGC | 15652 |
| 1052 | ACCCAGCA G CGGGAGGA | 6904 | TCCTCCCG GGCTAGCTACAACGA TGCTGGGT | 15653 |
| 1043 | CGGGAGGA G UUGUUCUC | 6905 | GAGAACAA GGCTAGCTACAACGA TCCTCCCG | 15654 |
| 1040 | GAGGAGUU G UUCUCCCG | 6906 | CGGGAGAA GGCTAGCTACAACGA AACTCCTC | 15655 |
| 1030 | UCUCCCGA A CGCAGGGC | 6907 | GCCCTGCG GGCTAGCTACAACGA TCGGGAGA | 15656 |
| 1028 | UCCCGAAC G CAGGGCAC | 6908 | GTGCCCTG GGCTAGCTACAACGA GTTCGGGA | 15657 |
| 1023 | AACGCAGG G CACGCACC | 6909 | GGTGCGTG GGCTAGCTACAACGA CCTGCGTT | 15658 |
| 1021 | CGCAGGGC A CGCACCCC | 6910 | GGGGTGCG GGCTAGCTACAACGA GCCCTGCG | 15659 |
| 1019 | CAGGGCAC G CACCCCGG | 6911 | CCGGGGTG GGCTAGCTACAACGA GTGCCCTG | 15660 |
| 1017 | GGGCACGC A CCCCGGGG | 6912 | CCCCGGGG GGCTAGCTACAACGA GCGTGCCC | 15661 |
| 1009 | ACCCCGGG G UGUGCAUG | 6913 | CATGCACA GGCTAGCTACAACGA CCCGGGGT | 15662 |
| 1007 | CCCGGGGU G UGCAUGAU | 6914 | ATCATGCA GGCTAGCTACAACGA ACCCCGGG | 15663 |
| 1005 | CGGGGUGU G CAUGAUCA | 6915 | TGATCATG GGCTAGCTACAACGA ACACCCCG | 15664 |
| 1003 | GGGUGUGC A UGAUCAUG | 6916 | CATGATCA GGCTAGCTACAACGA GCACACCC | 15665 |
| 1000 | UGUGCAUG A UCAUGUCC | 6917 | GGACATGA GGCTAGCTACAACGA CATGCACA | 15666 |
| 997 | GCAUGAUC A UGUCCUCU | 6918 | AGAGGACA GGCTAGCTACAACGA GATCATGC | 15667 |
| 995 | AUGAUCAU G UCCUCUGC | 6919 | GCAGAGGA GGCTAGCTACAACGA ATGATCAT | 15668 |
| 988 | UGUCCUCU G CCUCAUAC | 6920 | GTATGAGG GGCTAGCTACAACGA AGAGGACA | 15669 |
| 983 | UCUGCCUC A UACACAAU | 6921 | ATTGTGTA GGCTAGCTACAACGA GAGGCAGA | 15670 |
| 981 | UGCCUCAU A CACAAUGC | 6922 | GCATTGTG GGCTAGCTACAACGA ATGAGGCA | 15671 |
| 979 | CCUCAUAC A CAAUGCUU | 6923 | AAGCATTG GGCTAGCTACAACGA GTATGAGG | 15672 |
| 976 | CAUACACA A UGCUUGAG | 6924 | CTCAAGCA GGCTAGCTACAACGA TGTGTATG | 15673 |
| 974 | UACACAAU G CUUGAGUU | 6925 | AACTCAAG GGCTAGCTACAACGA ATTGTGTA | 15674 |
| 968 | AUGCUUGA G UUGGAGCA | 6926 | TGCTCCAA GGCTAGCTACAACGA TCAAGCAT | 15675 |
| 962 | GAGUUGGA G CAAUCGUU | 6927 | AACGATTG GGCTAGCTACAACGA TCCAACTC | 15676 |
| 959 | UUGGAGCA A UCGUUCGU | 6928 | ACGAACGA GGCTAGCTACAACGA TGCTCCAA | 15677 |
| 956 | GAGCAAUC G UUCGUGAC | 6929 | GTCACGAA GGCTAGCTACAACGA GATTGCTC | 15678 |
| 952 | AAUCGUUC G UGACAUGG | 6930 | CCATGTCA GGCTAGCTACAACGA GAACGATT | 15679 |
| 949 | CGUUCGUG A CAUGGUAC | 6931 | GTACCATG GGCTAGCTACAACGA CACGAACG | 15680 |
| 947 | UUCGUGAC A UGGUACAG | 6932 | CTGTACCA GGCTAGCTACAACGA GTCACGAA | 15681 |
| 944 | GUGACAUG G UACAGCCC | 6933 | GGGCTGTA GGCTAGCTACAACGA CATGTCAC | 15682 |
| 942 | GACAUGGU A CAGCCCGG | 6934 | CCGGGCTG GGCTAGCTACAACGA ACCATGTC | 15683 |
| 939 | AUGGUACA G CCCGGACG | 6935 | CGTCCGGG GGCTAGCTACAACGA TGTACCAT | 15684 |
| 933 | CAGCCCGG A CGCGUUGC | 6936 | GCAACGCG GGCTAGCTACAACGA CCGGGCTG | 15685 |
| 931 | GCCCGGAC G CGUUGCAC | 6937 | GTGCAACG GGCTAGCTACAACGA GTCCGGGC | 15686 |
| 929 | CCGGACGC G UUGCACAC | 6938 | GTGTGCAA GGCTAGCTACAACGA GCGTCCGG | 15687 |
| 926 | GACGCGUU G CACACCUC | 6939 | GAGGTGTG GGCTAGCTACAACGA AACGCGTC | 15688 |
| 924 | CGCGUUGC A CACCUCAU | 6940 | ATGAGGTG GGCTAGCTACAACGA GCAACGCG | 15689 |
| 922 | CGUUGCAC A CCUCAUAA | 6941 | TTATGAGG GGCTAGCTACAACGA GTGCAACG | 15690 |
| 917 | CACACCUC A UAAGCGGA | 6942 | TCCGCTTA GGCTAGCTACAACGA GAGGTGTG | 15691 |
| 913 | CCUCAUAA G CGGAGGCU | 6943 | AGCCTCCG GGCTAGCTACAACGA TTATGAGG | 15692 |
| 907 | AAGCGGAG G CUGGGAUG | 6944 | CATCCCAG GGCTAGCTACAACGA CTCCGCTT | 15693 |
| 901 | AGGCUGGG A UGGUCAGA | 6945 | TCTGACCA GGCTAGCTACAACGA CCCAGCCT | 15694 |
| 898 | CUGGGAUG G UCAGACAG | 6946 | CTGTCTGA GGCTAGCTACAACGA CATCCCAG | 15695 |
| 893 | AUGGUCAG A CAGGGCAG | 6947 | CTGCCCTG GGCTAGCTACAACGA CTGACCAT | 15696 |
| 888 | CAGACAGG G CAGCAGAG | 6948 | CTCTGCTG GGCTAGCTACAACGA CCTGTCTG | 15697 |
| 885 | ACAGGGCA G CAGAGCCA | 6949 | TGGCTCTG GGCTAGCTACAACGA TGCCCTGT | 15698 |
| 880 | GCAGCAGA G CCAAGAGG | 6950 | CCTCTTGG GGCTAGCTACAACGA TCTGCTGC | 15699 |
| 868 | AGAGGAAG A UAGAGAAA | 6951 | TTTCTCTA GGCTAGCTACAACGA CTTCCTCT | 15700 |
| 857 | GAGAAAGA G CAACCGGG | 6952 | CCCGGTTG GGCTAGCTACAACGA TCTTTCTC | 15701 |
| 854 | AAAGAGCA A CCGGGCAG | 6953 | CTGCCCGG GGCTAGCTACAACGA TGCTCTTT | 15702 |
| 849 | GCAACCGG G CAGAUUCC | 6954 | GGAATCTG GGCTAGCTACAACGA CCGGTTGC | 15703 |
| 845 | CCGGGCAG A UUCCCUGU | 6955 | ACAGGGAA GGCTAGCTACAACGA CTGCCCGG | 15704 |
| 838 | GAUUCCCU G UUGCAUAG | 6956 | CTATGCAA GGCTAGCTACAACGA AGGGAATC | 15705 |
| 835 | UCCCUGUU G CAUAGUUC | 6957 | GAACTATG GGCTAGCTACAACGA AACAGGGA | 15706 |
| 833 | CCUGUUGC A UAGUUCAC | 6958 | GTGAACTA GGCTAGCTACAACGA GCAACAGG | 15707 |
| 830 | GUUGCAUA G UUCACGCC | 6959 | GGCGTGAA GGCTAGCTACAACGA TATGCAAC | 15708 |
| 826 | CAUAGUUC A CGCCGUCU | 6960 | AGACGGCG GGCTAGCTACAACGA GAACTATG | 15709 |
| 824 | UAGUUCAC G CCGUCUUC | 6961 | GAAGACGG GGCTAGCTACAACGA GTGAACTA | 15710 |
| 821 | UUCACGCC G UCUUCCAG | 6962 | CTGGAAGA GGCTAGCTACAACGA GGCGTGAA | 15711 |
| 811 | CUUCCAGA A CCCGGACG | 6963 | CGTCCGGG GGCTAGCTACAACGA TCTGGAAG | 15712 |
| 805 | GAACCCGG A CGCCAUGC | 6964 | GCATGGCG GGCTAGCTACAACGA CCGGGTTC | 15713 |
| 803 | ACCCGGAC G CCAUGCGC | 6965 | GCGCATGG GGCTAGCTACAACGA GTCCGGGT | 15714 |
| 800 | CGGACGCC A UGCGCCAG | 6966 | CTGGCGCA GGCTAGCTACAACGA GGCGTCCG | 15715 |
| 798 | GACGCCAU G CGCCAGGG | 6967 | CCCTGGCG GGCTAGCTACAACGA ATGGCGTC | 15716 |
| 796 | CGCCAUGC G CCAGGGCC | 6968 | GGCCCTGG GGCTAGCTACAACGA GCATGGCG | 15717 |
| 790 | GCGCCAGG G CCCUGGCA | 6969 | TGCCAGGG GGCTAGCTACAACGA CCTGGCGC | 15718 |
| 784 | GGGCCCUG G CAGUGCCU | 6970 | AGGCACTG GGCTAGCTACAACGA CAGGGCCC | 15719 |
| 781 | CCCUGGCA G UGCCUCCC | 6971 | GGGAGGCA GGCTAGCTACAACGA TGCCAGGG | 15720 |
| 779 | CUGGCAGU G CCUCCCAA | 6972 | TTGGGAGG GGCTAGCTACAACGA ACTGCCAG | 15721 |
| 766 | CCAAGGGG G CGCCGACG | 6973 | CGTCGGCG GGCTAGCTACAACGA CCCCTTGG | 15722 |
| 764 | AAGGGGGC G CCGACGAG | 6974 | CTCGTCGG GGCTAGCTACAACGA GCCCCCTT | 15723 |
| 760 | GGGCGCCG A CGAGCGGA | 6975 | TCCGCTCG GGCTAGCTACAACGA CGGCGCCC | 15724 |
| 756 | GCCGACGA G CGGAAUGU | 6976 | ACATTCCG GGCTAGCTACAACGA TCGTCGGC | 15725 |
| 751 | CGAGCGGA A UGUACCCC | 6977 | GGGGTACA GGCTAGCTACAACGA TCCGCTCG | 15726 |
| 749 | AGCGGAAU G UACCCCAU | 6978 | ATGGGGTA GGCTAGCTACAACGA ATTCCGCT | 15727 |
| 747 | CGGAAUGU A CCCCAUGA | 6979 | TCATGGGG GGCTAGCTACAACGA ACATTCCG | 15728 |
| 742 | UGUACCCC A UGAGGUCG | 6980 | CGACCTCA GGCTAGCTACAACGA GGGGTACA | 15729 |
| 737 | CCCAUGAG G UCGGCGAA | 6981 | TTCGCCGA GGCTAGCTACAACGA CTCATGGG | 15730 |
| 733 | UGAGGUCG G CGAAGCCG | 6982 | CGGCTTCG GGCTAGCTACAACGA CGACCTCA | 15731 |
| 728 | UCGGCGAA G CCGCAUGU | 6983 | ACATGCGG GGCTAGCTACAACGA TTCGCCGA | 15732 |
| 725 | GCGAAGCC G CAUGUGAG | 6984 | CTCACATG GGCTAGCTACAACGA GGCTTCGC | 15733 |
| 723 | GAAGCCGC A UGUGAGGG | 6985 | CCCTCACA GGCTAGCTACAACGA GCGGCTTC | 15734 |
| 721 | AGCCGCAU G UGAGGGUA | 6986 | TACCCTCA GGCTAGCTACAACGA ATGCGGCT | 15735 |
| 715 | AUGUGAGG G UAUCGAUG | 6987 | CATCGATA GGCTAGCTACAACGA CCTCACAT | 15736 |
| 713 | GUGAGGGU A UCGAUGAC | 6988 | GTCATCGA GGCTAGCTACAACGA ACCCTCAC | 15737 |
| 709 | GGGUAUCG A UGACCUUA | 6989 | TAAGGTCA GGCTAGCTACAACGA CGATACCC | 15738 |
| 706 | UAUCGAUG A CCUUACCC | 6990 | GGGTAAGG GGCTAGCTACAACGA CATCGATA | 15739 |
| 701 | AUGACCUU A CCCAAGUU | 6991 | AACTTGGG GGCTAGCTACAACGA AAGGTCAT | 15740 |
| 695 | UUACCCAA G UUACGCGA | 6992 | TCGCGTAA GGCTAGCTACAACGA TTGGGTAA | 15741 |
| 692 | CCCAACUU A CGCGACCU | 6993 | AGGTCGCG GGCTAGCTACAACGA AACTTGGG | 15742 |
| 690 | GAACUUAG G CGACCUAC | 6994 | GTAGGTCG GGCTAGCTACAACGA GTAACTTG | 15743 |
| 687 | GUUACGCG A CCUACGCC | 6995 | GGCGTAGG GGCTAGCTACAACGA CGCGTAAC | 15744 |
| 683 | CGCGACCU A CGCCGGGG | 6996 | CCCCGGCG GGCTAGCTACAACGA AGGTCGCG | 15745 |
| 681 | CGACCUAC G CCGGGGGU | 6997 | ACCCCCGG GGCTAGCTACAACGA GTAGGTCG | 15746 |
| 674 | CGCCGGGG G UCCGUGGG | 6998 | CCCACGGA GGCTAGCTACAACGA CCCCGGCG | 15747 |
| 670 | GGGGGUCC G UGGGGCCC | 6999 | GGGCCCCA GGCTAGCTACAACGA GGACCCCC | 15748 |
| 665 | UCCGUGGG G CCCCAACU | 7000 | AGTTGGGG GGCTAGCTACAACGA CCCACGGA | 15749 |
| 659 | GGGCCCCA A CUAGGCCG | 7001 | CGGCCTAG GGCTAGCTACAACGA TGGGGCCC | 15750 |
| 654 | CCAACUAG G CCGGGAGC | 7002 | GCTCCCGG GGCTAGCTACAACGA CTAGTTGG | 15751 |
| 647 | GGCCGGGA G CCGCGGGG | 7003 | CCCCGCGG GGCTAGCTACAACGA TCCCGGCC | 15752 |
| 644 | CGGGAGCC G CGGGGUGA | 7004 | TCACCCCG GGCTAGCTACAACGA GGCTCCCG | 15753 |
| 639 | GCCGCGGG G UGACAGGA | 7005 | TCCTGTCA GGCTAGCTACAACGA CCCGCGGC | 15754 |
| 636 | GCGGGGUG A CAGGAGCC | 7006 | GGCTCCTG GGCTAGCTACAACGA CACCCCGC | 15755 |
| 630 | UGACAGGA G CCAUCCUG | 7007 | CAGGATGG GGCTAGCTACAACGA TCCTGTCA | 15756 |
| 627 | CAGGAGCC A UCCUGCCC | 7008 | GGGCAGGA GGCTAGCTACAACGA GGCTCCTG | 15757 |
| 622 | GCCAUCCU G CCCACCCU | 7009 | AGGGTGGG GGCTAGCTACAACGA AGGATGGC | 15758 |
| 618 | UCCUGCCC A CCCUAAGC | 7010 | GCTTAGGG GGCTAGCTACAACGA GGGCAGGA | 15759 |
| 611 | CACCCUAA G CCCUCAUU | 7011 | AATGAGGG GGCTAGCTACAACGA TTAGGGTG | 15760 |
| 605 | AAGCCCUC A UUGCCAUA | 7012 | TATGGCAA GGCTAGCTACAACGA GAGGGCTT | 15761 |
| 602 | CCCUCAUU G CCAUAGAG | 7013 | CTCTATGG GGCTAGCTACAACGA AATGAGGG | 15762 |
| 599 | UCAUUGCC A UAGAGGGG | 7014 | CCCCTCTA GGCTAGCTACAACGA GGCAATGA | 15763 |
| 591 | AUAGAGGG G CCAAGGGU | 7015 | ACCCTTGG GGCTAGCTACAACGA CCCTCTAT | 15764 |
| 584 | GGCCAAGG G UACCCGGG | 7016 | CCCGGGTA GGCTAGCTACAACGA CCTTGGCC | 15765 |
| 582 | CCAAGGGU A CCCGGGCU | 7017 | AGCCCGGG GGCTAGCTACAACGA ACCCTTGG | 15766 |
| 576 | GUACCCGG G CUGAGCCC | 7018 | GGGCTCAG GGCTAGCTACAACGA CCGGGTAC | 15767 |
| 571 | CGGGCUGA G CCCAGGCC | 7019 | GGCCTGGG GGCTAGCTACAACGA TCAGCCCG | 15768 |
| 565 | GAGCCCAG G CCCUGCCC | 7020 | GGGCAGGG GGCTAGCTACAACGA CTGGGCTC | 15769 |
| 560 | CAGGCCCU G CCCUCGGG | 7021 | CCCGAGGG GGCTAGCTACAACGA AGGGCCTG | 15770 |
| 552 | GCCCUCGG G CCGGCGAG | 7022 | CTCGCCGG GGCTAGCTACAACGA CCGAGGGC | 15771 |
| 548 | UCGGGCCG G CGAGCCUU | 7023 | AAGGCTCG GGCTAGCTACAACGA CGGCCCGA | 15772 |
| 544 | GCCGGCGA G CCUUGGGG | 7024 | CCCCAAGG GGCTAGCTACAACGA TCGCCGGC | 15773 |
| 535 | CCUUGGGG A UAGGUUGU | 7025 | ACAACCTA GGCTAGCTACAACGA CCCCAAGG | 15774 |
| 531 | GGGGAUAG G UUGUCGCC | 7026 | GGCGACAA GGCTAGCTACAACGA CTATCCCC | 15775 |
| 528 | GAUAGGUU G UCGCCUUC | 7027 | GAAGGCGA GGCTAGCTACAACGA AACCTATC | 15776 |
| 525 | AGGUUGUC G CCUUCCAC | 7028 | GTGGAAGG GGCTAGCTACAACGA GACAACCT | 15777 |
| 518 | CGCCUUCC A CGAGGUUG | 7029 | CAACCTCG GGCTAGCTACAACGA GGAAGGCG | 15778 |
| 513 | UCCACGAG G UUGCGACC | 7030 | GGTCGCAA GGCTAGCTACAACGA CTCGTGGA | 15779 |
| 510 | ACGAGGUU G CGACCGCU | 7031 | AGCGGTCG GGCTAGCTACAACGA AACCTCGT | 15780 |
| 507 | AGGUUGCG A CCGCUCGG | 7032 | CCGAGCGG GGCTAGCTACAACGA CGCAACCT | 15781 |
| 504 | UUGCGACC G CUCGGAAG | 7033 | CTTCCGAG GGCTAGCTACAACGA GGTCGCAA | 15782 |
| 496 | GCUCGGAA G UCUUCCUA | 7034 | TAGGAAGA GGCTAGCTACAACGA TTCCGAGC | 15783 |
| 487 | UCUUCCUA G UCGCGCGC | 7035 | GCGCGCGA GGCTAGCTACAACGA TAGGAAGA | 15784 |
| 484 | UCCUAGUC G CGCGCACA | 7036 | TGTGCGCG GGCTAGCTACAACGA GACTAGGA | 15785 |
| 482 | CUAGUCGC G CGCACACC | 7037 | GGTGTGCG GGCTAGCTACAACGA GCGACTAG | 15786 |
| 480 | AGUCGCGC G CACACCCA | 7038 | TGGGTGTG GGCTAGCTACAACGA GCGCGACT | 15787 |
| 478 | UCGCGCGC A CACCCAAC | 7039 | GTTGGGTG GGCTAGCTACAACGA GCGCGCGA | 15788 |
| 476 | GCGCGCAC A CCCAACCU | 7040 | AGGTTGGG GGCTAGCTACAACGA GTGCGCGC | 15789 |
| 471 | CACACCCA A CCUGGGGC | 7041 | GCCCCAGG GGCTAGCTACAACGA TGGGTGTG | 15790 |
| 464 | AACCUGGG G CCCCUGCG | 7042 | CGCAGGGG GGCTAGCTACAACGA CCCAGGTT | 15791 |
| 458 | GGGCCCCU G CGCGGCAA | 7043 | TTGCCGCG GGCTAGCTACAACGA AGGGGCCC | 15792 |
| 456 | GCCCCUGC G CGGCAACA | 7044 | TGTTGCCG GGCTAGCTACAACGA GCAGGGGC | 15793 |
| 453 | CCUGCGCG G CAACAGGU | 7045 | ACCTGTTG GGCTAGCTACAACGA CGCGCAGG | 15794 |
| 450 | GCGCGGCA A CAGGUAAA | 7046 | TTTACCTG GGCTAGCTACAACGA TGCCGCGC | 15795 |
| 446 | GGCAACAG G UAAACUCC | 7047 | GGAGTTTA GGCTAGCTACAACGA CTGTTGCC | 15796 |
| 442 | ACAGGUAA A CUCCACCA | 7048 | TGGTGGAG GGCTAGCTACAACGA TTACCTGT | 15797 |
| 437 | UAAACUCC A CCAACGAU | 7049 | ATCGTTGG GGCTAGCTACAACGA GGAGTTTA | 15798 |
| 433 | CUCCACCA A CGAUCUGA | 7050 | TCAGATCG GGCTAGCTACAACGA TGGTGGAG | 15799 |
| 430 | CACCAACG A UCUGACCA | 7051 | TGGTCAGA GGCTAGCTACAACGA CGTTGGTG | 15800 |
| 425 | ACGAUCUG A CCACCGCC | 7052 | GGCGGTGG GGCTAGCTACAACGA CAGATCGT | 15801 |
| 422 | AUCUGACC A CCGCCCGG | 7053 | CCGGGCGG GGCTAGCTACAACGA GGTCAGAT | 15802 |
| 419 | UGACCACC G CCCGGGAA | 7054 | TTCCCGGG GGCTAGCTACAACGA GGTGGTCA | 15803 |
| 411 | GCCCGGGA A CUUGACGU | 7055 | ACGTCAAG GGCTAGCTACAACGA TCCCGGGC | 15804 |
| 406 | GGAACUUG A CGUCCUGU | 7056 | ACAGGACG GGCTAGCTACAACGA CAAGTTCC | 15805 |
| 404 | AACUUGAC G UCCUGUGG | 7057 | CCACAGGA GGCTAGCTACAACGA GTCAAGTT | 15806 |
| 399 | GACGUCCU G UGGGCGGC | 7058 | GCCGCCCA GGCTAGCTACAACGA AGGACGTC | 15807 |
| 395 | UCCUGUGG G CGGCGGUU | 7059 | AACCGCCG GGCTAGCTACAACGA CCACAGGA | 15808 |
| 392 | UGUGGGCG G CGGUUGGU | 7060 | ACCAACCG GGCTAGCTACAACGA CGCCCACA | 15809 |
| 389 | GGGCGGCG G UUGGUGUU | 7061 | AACACCAA GGCTAGCTACAACGA CGCCGCCC | 15810 |
| 385 | GGCGGUUG G UGUUACGU | 7062 | ACGTAACA GGCTAGCTACAACGA CAACCGCC | 15811 |
| 383 | CGGUUGGU G UUACGUUU | 7063 | AAACGTAA GGCTAGCTACAACGA ACCAACCG | 15812 |
| 380 | UUGGUGUU A CGUUUGGU | 7064 | ACCAAACG GGCTAGCTACAACGA AACACCAA | 15813 |
| 378 | GGUGUUAC G UUUGGUUU | 7065 | AAACCAAA GGCTAGCTACAACGA GTAACACC | 15814 |
| 373 | UACGUUUG G UUUUUCUU | 7066 | AAGAAAAA GGCTAGCTACAACGA CAAACGTA | 15815 |
| 360 | UCUUUGAG G UUUAGGAU | 7067 | ATCCTAAA GGCTAGCTACAACGA CTCAAAGA | 15816 |
| 353 | GGUUUAGG A UUCGUGCU | 7068 | AGCACGAA GGCTAGCTACAACGA CCTAAACC | 15817 |
| 349 | UAGGAUUC G UGCUCAUG | 7069 | CATGAGCA GGCTAGCTACAACGA GAATCCTA | 15818 |
| 347 | GGAUUCGU G CUCAUGGU | 7070 | ACCATGAG GGCTAGCTACAACGA ACGAATCC | 15819 |
| 343 | UCGUGCUC A UGGUGCAC | 7071 | GTGCACCA GGCTAGCTACAACGA GAGCACGA | 15820 |
| 340 | UGCUCAUG G UGCACGGU | 7072 | ACCGTGCA GGCTAGCTACAACGA CATGAGCA | 15821 |
| 338 | CUCAUGGU G CACGGUCU | 7073 | AGACCGTG GGCTAGCTACAACGA ACCATGAG | 15822 |
| 336 | CAUGGUGC A CGGUCUAC | 7074 | GTAGACCG GGCTAGCTACAACGA GCACCATG | 15823 |
| 333 | GGUGCACG G UCUACGAG | 7075 | CTCGTAGA GGCTAGCTACAACGA CGTGCACC | 15824 |
| 329 | CACGGUCU A CGAGACCU | 7076 | AGGTCTCG GGCTAGCTACAACGA AGACCGTG | 15825 |
| 324 | UCUACGAG A CCUCCCGG | 7077 | CCGGGAGG GGCTAGCTACAACGA CTCGTAGA | 15826 |
| 314 | CUCCCGGG G CACUCGCA | 7078 | TGCGAGTG GGCTAGCTACAACGA CCCGGGAG | 15827 |
| 312 | CCCGGGGC A CUCGCAAG | 7079 | CTTGCGAG GGCTAGCTACAACGA GCCCCGGG | 15828 |
| 308 | GGGCACUC G CAAGCACC | 7080 | GGTGCTTG GGCTAGCTACAACGA GAGTGCCC | 15829 |
| 304 | ACUCGCAA G CACCCUAU | 7081 | ATAGGGTG GGCTAGCTACAACGA TTGCGAGT | 15830 |
| 302 | UCGCAAGC A CCCUAUCA | 7082 | TGATAGGG GGCTAGCTACAACGA GCTTGCGA | 15831 |
| 297 | AGCACCCU A UCAGGCAG | 7083 | CTGCCTGA GGCTAGCTACAACGA AGGGTGCT | 15832 |
| 292 | CCUAUCAG G CAGUACCA | 7084 | TGGTACTG GGCTAGCTACAACGA CTGATAGG | 15833 |
| 289 | AUCAGGCA G UACCACAA | 7085 | TTGTGGTA GGCTAGCTACAACGA TGCCTGAT | 15834 |
| 287 | CAGGCAGU A CCACAAGG | 7086 | CCTTGTGG GGCTAGCTACAACGA ACTGCCTG | 15835 |
| 284 | CGACUAGG A CAAGGCCU | 7087 | AGGCCTTG GGCTAGCTACAACGA GGTACTGC | 15836 |
| 279 | ACCACAAG G CCUUUCGC | 7088 | GCGAAAGG GGCTAGCTACAACGA CTTGTGGT | 15837 |
| 272 | GGCCUUUC G CGACCCAA | 7089 | TTGGGTCG GGCTAGCTACAACGA GAAAGGCC | 15838 |
| 269 | CUUUCGCG A CCCAACAC | 7090 | GTGTTGGG GGCTAGCTACAACGA CGCGAAAG | 15839 |
| 264 | GCGACCCA A CACUACUC | 7091 | GAGTAGTG GGCTAGCTACAACGA TGGGTCGC | 15840 |
| 262 | GACCCAAC A CUACUCGG | 7092 | CCGAGTAG GGCTAGCTACAACGA GTTGGGTC | 15841 |
| 259 | CCAACACU A CUCGGCUA | 7093 | TAGCCGAG GGCTAGCTACAACGA AGTGTTGG | 15842 |
| 254 | ACUACUCG G CUAGCAGU | 7094 | ACTGCTAG GGCTAGCTACAACGA CGAGTAGT | 15843 |
| 250 | CUCGGCUA G CAGUCUCG | 7095 | CGAGACTG GGCTAGCTACAACGA TAGCCGAG | 15844 |
| 247 | GGCUAGCA G UCUCGCGG | 7096 | CCGCGAGA GGCTAGCTACAACGA TGCTAGCC | 15845 |
| 242 | GCAGUCUC G CGGGGGCA | 7097 | TGCCCCCG GGCTAGCTACAACGA GAGACTGC | 15846 |
| 236 | UCGCGGGG G CACGCCCA | 7098 | TGGGCGTG GGCTAGCTACAACGA CCCCGCGA | 15847 |
| 234 | GCGGGGGC A CGCCCAAA | 7099 | TTTGGGCG GGCTAGCTACAACGA GCCCCCGC | 15848 |
| 232 | GGGGGCAC G CCCAAAUC | 7100 | GATTTGGG GGCTAGCTACAACGA GTGCCCCC | 15849 |
| 226 | ACGCCCAA A UCUCCAGG | 7101 | CCTGGAGA GGCTAGCTACAACGA TTGGGCGT | 15850 |
| 218 | AUCUCCAG G CAUUGAGC | 7102 | GCTCAATG GGCTAGCTACAACGA CTGGAGAT | 15851 |
| 216 | CUCCAGGC A UUGAGCGG | 7103 | CCGCTCAA GGCTAGCTACAACGA GCCTGGAG | 15852 |
| 211 | GGCAUUGA G CGGGUUGA | 7104 | TCAACCCG GGCTAGCTACAACGA TCAATGCC | 15853 |
| 207 | UUGAGCGG G UUGAUCCA | 7105 | TGGATCAA GGCTAGCTACAACGA CCGCTCAA | 15854 |
| 203 | GCGGGUUG A UCCAAGAA | 7106 | TTCTTGGA GGCTAGCTACAACGA CAACCCGC | 15855 |
| 191 | AAGAAAGG A CCCGGUCG | 7107 | CGACCGGG GGCTAGCTACAACGA CCTTTCTT | 15856 |
| 186 | AGGACCCG G UCGUCCUG | 7108 | CAGGACGA GGCTAGCTACAACGA CGGGTCCT | 15857 |
| 183 | ACCCGGUC G UCCUGGCA | 7109 | TGCCAGGA GGCTAGCTACAACGA GACCGGGT | 15858 |
| 177 | UCGUCCUG G CAAUUCCG | 7110 | CGGAATTG GGCTAGCTACAACGA CAGGACGA | 15859 |
| 174 | UCCUGGCA A UUCCGGUG | 7111 | CACCGGAA GGCTAGCTACAACGA TGCCAGGA | 15860 |
| 168 | CAAUUCCG G UGUACUCA | 7112 | TGAGTACA GGCTAGCTACAACGA CGGAATTG | 15861 |
| 166 | AUUCCGGU G UACUCACC | 7113 | GGTGAGTA GGCTAGCTACAACGA ACCGGAAT | 15862 |
| 164 | UCCGGUGU A CUCACCGG | 7114 | CCGGTGAG GGCTAGCTACAACGA ACACCGGA | 15863 |
| 160 | GUGUACUC A CCGGUUCC | 7115 | GGAACCGG GGCTAGCTACAACGA GAGTACAC | 15864 |
| 156 | ACUCACCG G UUCCGCAG | 7116 | CTGCGGAA GGCTAGCTACAACGA CGGTGAGT | 15865 |
| 151 | CCGGUUCC G CAGACCAC | 7117 | GTGGTCTG GGCTAGCTACAACGA GGAACCGG | 15866 |
| 147 | UUCCGCAG A CCACUAUG | 7118 | CATAGTGG GGCTAGCTACAACGA CTGCGGAA | 15867 |
| 144 | CGCAGACC A CUAUGGCU | 7119 | AGCCATAG GGCTAGCTACAACGA GGTCTGCG | 15868 |
| 141 | AGACCACU A UGGCUCUC | 7120 | GAGAGCCA GGCTAGCTACAACGA AGTGGTCT | 15869 |
| 138 | CCACUAUG G CUCUCCCG | 7121 | CGGGAGAG GGCTAGCTACAACGA CATAGTGG | 15870 |
| 120 | GAGGGGGG G UCCUGGAG | 7122 | CTCCAGGA GGCTAGCTACAACGA CCCCCCTC | 15871 |
| 111 | UCCUGGAG G CUGCACGA | 7123 | TCGTGCAG GGCTAGCTACAACGA CTCCAGGA | 15872 |
| 108 | UGGAGGCU G CACGACAC | 7124 | GTGTCGTG GGCTAGCTACAACGA AGCCTCCA | 15873 |
| 106 | GAGGCUGC A CGACACUC | 7125 | GAGTGTCG GGCTAGCTACAACGA GCAGCCTC | 15874 |
| 103 | GCUGCACG A CACUCAUA | 7126 | TATGAGTG GGCTAGCTACAACGA CGTGCAGC | 15875 |
| 101 | UGCACGAC A CUCAUACU | 7127 | AGTATGAG GGCTAGCTACAACGA GTCGTGCA | 15876 |
| 97 | CGACACUC A UACUAACG | 7128 | CGTTAGTA GGCTAGCTACAACGA GAGTGTCG | 15877 |
| 95 | ACACUCAU A CUAACGCC | 7129 | GGCGTTAG GGCTAGCTACAACGA ATGAGTGT | 15878 |
| 91 | UCAUACUA A CGCCAUGG | 7130 | CCATGGCG GGCTAGCTACAACGA TAGTATGA | 15879 |
| 89 | AUACUAAC G CCAUGGCU | 7131 | AGCCATGG GGCTAGCTACAACGA GTTAGTAT | 15880 |
| 86 | CUAACGCC A UGGCUAGA | 7132 | TCTAGCCA GGCTAGCTACAACGA GGCGTTAG | 15881 |
| 83 | ACGCCAUG G CUAGACGC | 7133 | GCGTCTAG GGCTAGCTACAACGA CATGGCGT | 15882 |
| 78 | AUGGCUAG A CGCUUUCU | 7134 | AGAAAGCG GGCTAGCTACAACGA CTAGCCAT | 15883 |
| 76 | GGCUAGAC G CUUUCUGC | 7135 | GCAGAAAG GGCTAGCTACAACGA GTCTAGCC | 15884 |
| 69 | CGCUUUCU G CGUGAAGA | 7136 | TCTTCACG GGCTAGCTACAACGA AGAAAGCG | 15885 |
| 67 | CUUUCUGC G UGAAGACA | 7137 | TGTCTTCA GGCTAGCTACAACGA GCAGAAAG | 15886 |
| 61 | GCGUGAAG A CAGUAGUU | 7138 | AACTACTG GGCTAGCTACAACGA CTTCACGC | 15887 |
| 58 | UGAAGACA G UAGUUCCU | 7139 | AGGAACTA GGCTAGCTACAACGA TGTCTTCA | 15888 |
| 55 | AGACAGUA G UUCCUCAC | 7140 | GTGAGGAA GGCTAGCTACAACGA TACTGTCT | 15889 |
| 48 | AGUUCCUC A CAGGGGAG | 7141 | CTCCCCTG GGCTAGCTACAACGA GAGGAACT | 15890 |
| 40 | ACAGGGGA G UGAUCUAU | 7142 | ATAGATCA GGCTAGCTACAACGA TCCCCTCT | 15891 |
| 37 | GGGGAGUG A UCUAUGGU | 7143 | ACCATAGA GGCTAGCTACAACGA CACTCCCC | 15892 |
| 33 | AGUGAUCU A UGGUGGAG | 7144 | CTCCACCA GGCTAGCTACAACGA AGATCACT | 15893 |
| 30 | GAUCUAUG G UGGAGUGU | 7145 | ACACTCCA GGCTAGCTACAACGA CATAGATC | 15894 |
| 25 | AUGGUGGA G UGUCGCCC | 7146 | GGGCGACA GGCTAGCTACAACGA TCCACCAT | 15895 |
| 23 | GGUGGAGU G UCGCCCCC | 7147 | GGGGGCGA GGCTAGCTACAACGA ACTCCACC | 15896 |

Input Sequence = HPCK1S1.
Cut Site = R/Y
Arm Length = 8.
Core Sequence = GGCTAGCTACAACGA
HPCK1S1 Hepatitis G virus (strain HCV-1b, clone HCV-K1-S1), complete genome; acc# gi|1030702|dbj|D50483.1; 9410 nt

TABLE XX

| Synthetic anti-HCV nucleic acid molecule and Target Sequences | | | | | | | |
|---|---|---|---|---|---|---|---|
| ref pos | Ref Seq | Target | Seq ID | RPI# | NUCLEIC ACID | Seq ID | Nucleic Acid Alias |
| 195 | HCV+ | GGGUCCU U UCUUGCA | 7148 | 15364 | c_{S}c_{S}a_{S}a_{S}ga cUGAuGaggcgaaagccGaa uuAggacc B | 15897 | Hammerhead |
| 342 | HCV+ | AGACCGUGCAUCAUGAGCAC | 7149 | 17501 | GₛTₛGₛC_{S}TₛC_{S}A_{S}TₛGₛA_{S}TₛGₛC_{S}A_{S}C_{S}GₛGₛTₛC_{S}T | 15898 | Antisense |
| 195 | HCV+ | GGGUCCU U UCUUGGA | 7148 | 17558 | C_{S}C_{S}a_{S}a_{S}ga cUGAuGaggcguuagccGaZ Aggacc B | 15899 | Hammerhead |
| 195 | HCV+ | GGGUCCU U UCUUGGA | 7148 | 17559 | C_{S}c_{S}a_{S}a_{S}ga cUGAuGaggcguuagccGaa AggaZc B | 15900 | Hammerhead |
| 195 | HCV+ | GGGUCCU U UCUUGGA | 7148 | 17560 | Z5C_{S}a_{S}a_{S}ga cUGAuGaggcguuagccGaa Aggace B | 15901 | Hammerhead |
| 195 | HCV+ | GGGUCCU U UCUUGGA | 7148 | 17561 | Z C_{S}a_{S}a_{S}ga cUGAuGaggcguuagccCaa Aggacc B | 15902 | Hammerhead |
| 195 | HCV+ | GGGUCCU U UCUUGGA | 7148 | 18012 | ccaaga cUGAuGaggcguuagccGaa Aggaca B | 15903 | Hammerhead |
| 82 | HCV+ | GCGUCUA G CCAUGGC | 7150 | 18744 | g_{S}C_{S}C_{S}a_{S}ugg GccgaaagGCGaGucaaGGuCu uagacgc B | 15904 | Zinzyme |
| 100 | HCV+ | AGUAUGA G UGUCGUG | 7151 | 18745 | C_{S}a_{S}C_{S}g_{S}aca GccgaaagGCGaGucaaGGuCu ucauacu B | 15905 | Zinzyme |
| 102 | HCV+ | UAUGAGU G UCGUGCA | 7152 | 18746 | u_{S}g_{S}C_{S}a_{S}cga GccgaaagGCGaGucaaGGuCu acucaua B | 15906 | Zinzyme |
| 105 | HCV+ | GAGUGUC G UGCAGCC | 7153 | 18747 | g_{S}g_{S}C_{S}u_{S}gca GccgaaagGCGaGucaaGGuCu gacacuc B | 15907 | Zinzyme |
| 107 | HCV+ | GUGUCGU G CAGCCUC | 7154 | 18748 | g_{S}a_{S}g8g_{S}cug GccgaaagGCGaGucaaGGuCu acgacac B | 15908 | Zinzyme |
| 146 | HCV+ | CAUAGUG G UCUGCGG | 7155 | 18749 | C_{S}C_{S}g_{S}C_{S}aga GccgaaagGCGaGucaaGGuCu cacuaug B | 15909 | Zinzyme |
| 190 | HCV+ | CGACCGG G UCCUUUC | 7156 | 18750 | g_{S}a_{S}a_{S}a_{S}gga GccgaaagGCGaGucaaGGuCu ccggucg B | 15910 | Zinzyme |
| 217 | HCV+ | GCUCAAU G CCUGGAG | 7157 | 18751 | C_{S}u_{S}C_{S}C_{S}agg GccgaaagGCGaGucaaGGuCu auugagc B | 15911 | Zinzyme |
| 231 | HCV+ | GAUUUGG C CGUGCCC | 7158 | 18752 | g_{S}g_{S}g_{S}C_{S}acg GccgaaagGCCaGucaaGGuCu ccaaauc B | 15912 | Zinzyme |
| 258 | HCV+ | UAGCCGA C UAGUGUU | 7159 | 18753 | asascsascua GccgaaagGCGaGucaaGGuCu ucggcua B | 15913 | Zinzyme |
| 307 | HCV+ | GGUGCUU G CGAGUGC | 7160 | 18754 | g_{S}c_{S}a_{S}c9ucg GccgaaagGCGaGucaaGGuCu aagcacc B | 15914 | Zinzyme |
| 77 | HCV+ | GAAAGC G UCUAGC | 7161 | 18755 | g_{S}C_{S}u_{S}a_{S}ga GccgaaagGCGaGucaaGGuCu gcuuuc B | 15915 | Zinzyme |
| 77 | HCV+ | AGAAAGC G UCUAGCC | 7162 | 18756 | g_{S}g_{S}C_{S}u_{S}aga GccgaaagGCGaGucaaGGuCu gcuuucu B | 15916 | Zinzyme |
| 88 | HCV+ | AGCCAUG C CGUUAGU | 7163 | 18757 | a_{S}C_{S}u6a_{S}acg GccgaaagGCGaGucaaGGuCu cauggcu B | 15917 | Zinzyme |
| 94 | HCV+ | CGCGUUA C UAUGAGU | 7164 | 18758 | ascsuscsaua GccgaaagGCGaGucaaGGucu uaacgcc B | 15918 | Zinzyme |
| 102 | HCV+ | AUGAGU G UCGUGC | 7165 | 18759 | g_{S}C_{S}a_{S}c_{S}ga CccgaaagGCGaCucaaGGuCu acucau B | 15919 | Zinzyme |
| 105 | HCV+ | AGUGUC G UGCAGC | 7166 | 18760 | g_{S}c_{S}u_{S}g_{S}ca GccgaaagGCGaGucaaGGuCu gacacu B | 15920 | Zinzyme |
| 110 | HCV+ | UCGUGCA C CCUCCAG | 7167 | 18761 | C_{S}u_{S}g_{S}g_{S}agg GccgaaagCCGaGucaaGGuCu ugcacga B | 15921 | Zinzyme |
| 137 | HCV+ | GGGAGA G CCAUAG | 7168 | 18762 | C_{S}u_{S}a_{S}u_{S}gg GccgaaagGCGaGucaaGGuCu ucuccc B | 15922 | Zinzyme |
| 137 | HCV+ | CGGGAGA G CCAUAGU | 7169 | 18763 | a_{S}C_{S}u_{S}a_{S}ugg GccgaaagGCGaGucaaGGuCu ucucccg B | 15923 | Zinzyme |
| 146 | HCV+ | AUAGUG G UCUGCG | 7170 | 18764 | C_{S}g_{S}C_{S}a_{S}ga GccgaaagGCGaGucaaGGuCu cacuau B | 15924 | Zinzyme |
| 150 | HCV+ | GUGGUCU G CGGAACC | 7171 | 18765 | g_{S}g_{S}u_{S}u_{S}ccg GccgaaagGCGaGucaaGGuCu agaccac B | 15925 | Zinzyme |
| 176 | HCV+ | CGGAAUU C CCAGGAC | 7172 | 18766 | g_{S}u_{S}C_{S}c_{S}ugg GccgaaagCCCaCucaaGGuCu aauuccg B | 15926 | Zinzyme |
| 190 | HCV+ | GACCGG GUCCUUU | 7173 | 18767 | a_{S}a_{S}a_{S}g_{S}gaGccgaaagGCGaGucaaGGuCu ccgguc B | 15927 | Zinzyme |
| 253 | HSV+ | CUGCUA G CCGAGU | 7174 | 18768 | a_{S}C_{S}ugc_{S}ggGccgaaagGCGaGucaaGGuCu uagcag B | 15928 | Zinzyme |
| 253 | HCV+ | ACUGCUA GCCGAGAA | 7175 | 18769 | u_{S}a_{S}c_{S}u_{S}cggGccgaaagGCGaGucaaGGuCu uagcagu B | 15929 | Zinzyme |
| 258 | HCV+ | AGCCGA GUAGUGU | 7176 | 18770 | a_{S}C_{S}a_{S}c_{S}uaGccgaaagGCGaGucaaGGuCu ucggcu B | 15930 | Zinzyme |
| 263 | HCV+ | GAGUAGU GUUGGGUC | 7177 | 18771 | g_{S}a_{S}c_{S}c_{S}caaGccgaaagGcGaGucaaGGuCu acuacuc B | 15931 | Zinzyme |
| 268 | HCV+ | UGUUGG GUCGCGA | 7178 | 18772 | u_{S}c_{S}g_{S}c_{S}gaGccgaaagGCGaGucaaGGuCu ccaaca B | 15932 | Zinzyme |
| 268 | HCV+ | GUGUUGG GUCGCGAA | 7179 | 18773 | u_{S}u_{S}c_{S}g_{S}cgaGccgaaagGcGaGucaaGGuCu ccaacac B | 15933 | Zinzyme |
| 271 | HCV+ | UUGGGUC GCGAAAGG | 7180 | 18774 | c_{S}cgu_{S}u_{S}ucgGccgaaagGCGaGucaaGGuCu gacccaa B | 15934 | Zinzyme |
| 283 | HCV+ | AGGCCUU GUGGUACU | 7181 | 18775 | a_{S}g_{S}u_{S}a_{S}ccaGccgaaagGCGaGucaaGGuCu aaggccu B | 15935 | Zinzyme |
| 286 | HCV+ | CCUUGUG GUACUGCC | 7182 | 18776 | g_{S}g_{S}c_{S}a_{S}guaGccgaaagGCGacucaaGGuCu cacaagg B | 15936 | Zinzyme |
| 291 | HCV+ | UGGUACU GCCUGAUA | 7183 | 18777 | u_{S}a_{S}u_{S}C_{S}aggGccgaaagGCGaGucaaGGuCu aguacca B | 15937 | Zinzyme |
| 301 | HCV+ | UGAUAGG GUGCUUGC | 7184 | 18778 | g_{S}C_{S}a_{S}a_{S}gcaGccgaaagGCGaGucaaGGuCu ccuauca B | 15938 | Zinzyme |
| 303 | HCV+ | AUAGGGU GCUUGCGA | 7185 | 18779 | u_{S}c_{S}g_{S}c_{S}aagGccgaaagGCGaGucaaGGuCu acccuau B | 15939 | Zinzyme |
| 60 | HCV+ | ACUACU GUCUUCA | 7186 | 18780 | u_{S}g_{S}a_{S}a_{S}gaGccgaaagGCGaGucaaGGuCu aguagu B | 15940 | Zinzyme |
| 60 | HCV+ | AACUACU GUCUUCAC | 7187 | 18781 | g_{S}u_{S}g_{S}a_{S}agaGccgaaagGCGaGucaaGGuCu aguaguu B | 15941 | Zinzyme |
| 68 | HCV+ | UCUUCAC GCAGAAAG | 7188 | 18782 | c_{S}u_{S}u_{S}u_{S}cugGccgaaagGCGaGucaaGGuCu gugaaga B | 15942 | Zinzyme |
| 75 | HCV+ | CAGAAA GCGUCUA | 7189 | 18783 | u_{S}a_{S}g_{S}a_{S}cgGccgaaagGCGaGucaaGGuCu uuucug B | 15943 | Zinzyme |
| 82 | HCV+ | CGUCUA GCCAUGG | 7190 | 18784 | cgC_{S}a_{S}u_{S}ggGccgaaagGCGaGucaaGGuCu uagacg B | 15944 | Zirizyme |
| 88 | HCV+ | GCCAUG GCGUUAG | 7191 | 18785 | C_{S}u_{S}a_{S}a_{S}cgGccgaaagGCGaGucaacGuCu cauggc B | 15945 | Zinzyme |
| 90 | HCV+ | CAUGGC GUUAGUA | 7192 | 18786 | usascsusaaGccgaaagGCGaGucaaGGuCu gccaug B | 15946 | Zinzyme |
| 90 | HCV+ | CCAUGGC GUUAGUAU | 7193 | 18787 | a_{S}u_{S}a_{S}c_{S}uaaGccgaaagGCGaGucaaGGucu gccaugg B | 15947 | Zinzyme |
| 100 | HCV+ | GUAUGA G UGUCGU | 7194 | 18788 | a_{S}c_{S}g_{S}a_{S}caGccgaaagGCGaGucaaGGuCu ucauac B | 15948 | Zinzyme |
| 107 | HCV+ | UGUCGU GCAGCCU | 7195 | 18789 | a_{S}g_{S}g_{S}C_{S}ugGccgaaagGCGaGucaaGGuCu acgaca B | 15949 | Zinzyme |
| 110 | HCV+ | CGUGCA GCCUCCA | 7196 | 18790 | u_{S}g_{S}g_{S}a_{S}ggGccgaaagGCGaGucaaGGuCu ugcacg B | 15950 | Zinzyme |
| 150 | HCV+ | UGGUCU GCGGAAC | 7197 | 18791 | g_{S}u_{S}u_{S}C_{S}cgGccgaaagGCGaGucaaGGuCu agacca B | 15951 | Zinzyme |
| 159 | HCV+ | GGAACCG GUGAGUAC | 7198 | 18792 | g_{S}u_{S}a_{S}c_{S}ucaGccgaaagGCGaGucaaGGuCu cgguucc B | 15952 | Zinzyme |
| 176 | HCV+ | GGAAUU GCCAGGA | 7199 | 18793 | u_{S}c_{S}c_{S}u_{S}ggGccgaaagGCGaGucaaGGuCu aauucc B | 15953 | Zinzyme |
| 217 | HCV+ | CUCAAU GCCUGGA | 7200 | 18794 | u_{S}C_{S}c_{S}a_{S}ggGccgaaagGCGaGucaaGGuCu auugag B | 15954 | Zinzyme |
| 231 | HCV+ | AUUUGG GCGUGCC | 7201 | 18795 | g_{S}g_{S}c_{S}a_{S}cgGccgaaagGCGaGucaaGGuCu ccaaau B | 15955 | Zinzyme |
| 261 | HCV+ | CGAGUA GUGUUGG | 7202 | 18796 | c_{S}c_{S}a_{S}a_{S}caGccgaaagGCGaGucaaGGuCu uacucg B | 15956 | Zinzyme |
| 261 | HCV+ | CCGAGUA GUGUUGGG | 7203 | 18797 | C_{S}c_{S}C_{S}a_{S}acaGccgaaagGCGaGucaaGGuCu uacucgg B | 15957 | Zinzyrne |
| 263 | HCV+ | AGUAGU GUUGGGU | 7204 | 18798 | a_{S}c_{S}c_{S}c_{S}aaGccgaaagGCGaGucaaGGuCu acuacu B | 15958 | Zinzyrne |
| 271 | HCV+ | UGGGUC GCGAAAG | 7205 | 18799 | C_{S}u_{S}u_{S}u_{S}cgGccgaaagGCGaGucaaGGuCu gaccca B | 15959 | Zinzyme |
| 283 | HCV+ | GGCCUU GUGGUAC | 7206 | 18800 | g_{S}u_{S}a_{S}c_{S}caGccgaaagGCGaGucaaGGuCu aaggcc B | 15960 | Zinzyme |
| 291 | HCV+ | GGUACU GCCUGAU | 7207 | 18801 | a_{S}u_{S}c_{S}a_{S}ggGccgaaagGCGaGucaaGGuCu aguacc B | 15961 | Zinzyme |
| 303 | HCV+ | UAGGGU G CUGCG | 7208 | 18802 | C_{S}g_{S}c_{S}a_{S}agGccgaaagGCGaGucaaGGuCu acccua B | 15962 | Zinzyme |
| 307 | HCV+ | GUGCUU G CGAGUG | 7209 | 18803 | c_{S}a_{S}c_{S}u_{S}cgGccgaaagGCGaGucaaGGuCu aagcac B | 15963 | Zinzyme |
| 323 | HCV+ | CGGGAG G UCUCGU | 7210 | 18804 | a_{S}c_{S}g_{S}a_{S}gaGccgaaagGCGaGucaaGGuCu cucccg B | 15964 | Zinzyme |
| 323 | HCV+ | CCGGGAG G UCUCGUA | 7211 | 18805 | u_{S}a_{S}c_{S}g_{S}agaGccgaaagGCGaGucaaGGuCu cucccgg B | 15965 | Zinzyme |
| 75 | HCV+ | GCAGAAA G CGUCUAG | 7212 | 18806 | c_{S}u_{S}a_{S}g_{S}acgGccgaaagGCGaGucaaGGuCu uuucugc B | 15966 | Zinzyme |
| 143 | HCV+ | GCCAUA G UGGUCU | 7213 | 18807 | a_{S}g_{S}a_{S}C_{S}caGccgaaagGCGaGucaaGGuCu uauggc B | 15967 | Zinzyme |
| 278 | HCV+ | GCGAAAG G CCUUGUG | 7214 | 18808 | c_{S}a_{S}c_{S}a_{S}aggCCC gaaagGCGaGucaaGGucu cuuucgc B | 15968 | Zinzyme |
| 163 | HCV+ | CGGUGA G UACACC | 7215 | 18809 | g_{S}g_{S}u_{S}g_{S}uaCccgaaagGCGaGucaaGGuCu ucaccg B | 15969 | Zinzyme |
| 68 | HCV+ | CUUCAC C CAGAAA | 7216 | 18810 | u_{S}u_{S}u_{S}c_{S}ugCccgaaagGCCaGucaaCGuCu gugaag B | 15970 | Zinzyme |
| 94 | HCV+ | CCGUUA G UAUGAC | 7217 | 18811 | c_{S}u_{S}C_{S}a_{S}uaCccgaaagCCCaCucaaGCuCu uaacgc B | 15971 | Zinzyme |
| 143 | HCV+ | ACCCAUA G UGCUCUC | 7218 | 18812 | c_{S}a_{S}g_{S}a_{S}ccaGccgaaagGCGaCucaaCCuCu uauggcu B | 15972 | Zinzyme |
| 159 | HCV+ | GAACCG G UGAGUA | 7219 | 18813 | u_{S}a_{S}c_{S}u_{S}caCccgaaagGCCaCucaaGGuCu cgguuc B | 15973 | Zinzyme |
| 163 | HCV+ | CCGGUCA G UACACCG | 7220 | 18814 | c_{S}g_{S}g_{S}u_{S}guaCccgaaagCCCaGucaaCGuCu ucaccgg B | 15974 | Zinzyme |
| 249 | HCV+ | CACACU C CUACCC | 7221 | 18815 | g_{S}g_{S}C_{S}u_{S}agCCcgaaagGCGaGucaaGGucu agucuc B | 15975 | Zinzyme |
| 249 | HCV+ | CCAGACU C CUAGCCG | 7222 | 18816 | c_{S}g_{S}g_{S}C_{S}uagGCCgaaagCCCaCUCaaGCUCU agucucg B | 15976 | Zinzyme |
| 278 | HCV+ | CGAAAC C CCUUCU | 7223 | 18817 | a_{S}c_{S}a_{S}a_{S}ggCccgaaagCCGaGucaaGGuCu cuuucg B | 15977 | Zinzyme |
| 286 | HCV+ | CUUCUC C UACUCC | 7224 | 18818 | g_{S}C_{S}a_{S}g_{S}uaCccgaaagCCGaCucaaGCuCu cacaag B | 15978 | Zinzyme |
| 301 | HCV+ | CAUACC C UCCUUC | 7225 | 18819 | c_{S}a_{S}a_{S}g_{S}caCccgaaagCCCaCucaaCCuCu ccuauc B | 15979 | Zinzyme |
| 328 | HCV+ | CCUCUC C UACACC | 7226 | 18820 | g_{S}g_{S}u_{S}C_{S}uaCccgaaagCCCaCucaaCCuCu gagacc B | 15980 | Zinzyme |
| 328 | HCV+ | ACCUCUC C UACACCC | 7227 | 18821 | c_{S}g_{S}g_{S}u_{S}cuaCccgaaagCCCaCucaaCCuCu gagaccu B | 15981 | Zinzyme |
| 335 | HCV+ | UACACC C UCCACC | 7228 | 18822 | g_{S}g_{S}u_{S}g_{S}caCccgaaagCCCaCucaaCCuCu ggucua B | 15982 | Zinzyme |
| 30 | C | UAAACCU C AAACAAA | 7229 | 19108 | u_{S}u_{S}u_{S}c_{S}uuucuCAuCaggccguuaggccCaa Agguuua B | 15983 | Hammerhead |
| 48 | C | CAAACCU A ACACCAA | 7230 | 19109 | u_{S}u_{S}g_{S}g_{S}ugucT3CAuCaggccguuaggccCaa Acguuug B | 15984 | Hammerhead |
| 60 | C | CAACCCU C CCCCACA | 7231 | 19110 | u_{S}g_{S}u_{S}g_{S}ggccUCAuCaggccguuaggccCaa Acgguug B | 15985 | Hammerhead |
| 175 | C | CACCCCU C ACAACCU | 7232 | 19111 | a_{S}g_{S}g_{S}u_{S}ugucuCAuCaggccguuaggccCaa Accgcuc B | 15986 | Hammerhead |
| 374 | C | CUAACCU C AUCCAUA | 7233 | 19112 | u_{S}a_{S}u_{S}c_{S}gaucUCAuCaggccguuaggccCaa Accuuac B | 15987 | Hammerhead |
| 258 | S27 | UCCUCCCUCCAUCUUACCCCUAC | 7234 | 22022 | u_{S}g_{S}g_{S}u_{S}g_{S}g_{S}c_{S}u_{S}c_{S}c_{S}a_{S}u_{S}c_{S}u_{S}u_{S}a_{S}g_{S}c_{S}c_{S}c_{S}u_{S}a_{S}g | 15988 | Antisense |
| 259 | S27 | CCUCCCUCCAUCUUACCCCUACU | 7235 | 22023 | g_{S}g_{S}u_{S}g_{S}g_{S}C_{S}u_{S}C_{S}C_{S}a_{S}u_{S}c_{S}u_{S}u_{S}a_{S}g_{S}C_{S}c_{S}c_{S}u_{S}a_{S}g_{S}u | 15989 | Antisense |
| 260 | S27 | CUCCCUCCAUCUUACCCCUACUC | 7236 | 22024 | g_{S}u_{S}g_{S}g_{S}C_{S}u_{S}c_{S}c_{S}a_{S}u_{S}c_{S}u_{S}u_{S}a_{S}g_{S}C_{S}c_{S}C_{S}u_{S}a_{S}g_{S}u_{S}c | 15990 | Antisense |
| 261 | S27 | UCCCUCCAUCUUACCCCUACUCA | 7237 | 22025 | u_{S}g_{S}g_{S}C_{S}u_{S}C_{S}C_{S}a_{S}u_{S}C_{S}u_{S}u_{S}a_{S}g_{S}C_{S}C_{S}C_{S}u_{S}a_{S}g_{S}u_{S}C_{S}a | 15991 | Antisense |
| 262 | S27 | CCCUCCAUCUUACCCCUACUCAC | 7238 | 22026 | g_{S}g_{S}C_{S}u_{S}C_{S}C_{S}a_{S}u_{S}C_{S}u_{S}u_{S}a_{S}g_{S}C_{S}C_{S}C_{S}u_{S}a_{S}g_{S}u_{S}C_{S}a_{S}c | 15992 | Antisense |
| 263 | S27 | CCUCCAUCUUACCCCUACUCACC | 7239 | 22027 | g_{S}C_{S}u_{S}C_{S}C_{S}a_{S}u_{S}C_{S}u_{S}u_{S}a_{S}g_{S}C_{S}C_{S}C_{S}u_{S}a_{S}g_{S}u_{S}C_{S}a_{S}c_{S}g | 15993 | Antisense |
| 264 | S27 | CUCCAUCUUACCCCUACUCACCC | 7240 | 22028 | C_{S}u_{S}c_{S}c_{S}a_{S}u_{S}C_{S}u_{S}u_{S}a_{S}g_{S}c_{S}C_{S}C_{S}u_{S}a_{S}g_{S}u_{S}C_{S}a_{S}C_{S}g_{S}g | 15994 | Antisense |
| 265 | S27 | UCCAUCUUACCCCUACUCACCCC | 7241 | 22029 | u_{S}C_{S}C_{S}a_{S}u_{S}C_{S}u_{S}u_{S}a_{S}g_{S}C_{S}C_{S}C_{S}u_{S}a_{S}g_{S}u_{S}C_{S}a_{S}C_{S}g_{S}g_{S}c | 15995 | Antisense |
| 266 | S27 | CCAUCUUACCCCUACUCACCCCU | 7242 | 22030 | C_{S}C_{S}a_{S}u_{S}C_{S}u_{S}u_{S}a_{S}g_{S}C_{S}C_{S}c_{S}u_{S}a_{S}g_{S}u_{S}C_{S}a_{S}C_{S}g_{S}g_{S}C_{S}u | 15996 | Antisense |
| 267 | S27 | CAUCUUAGCCCUAGUCACGGCUA | 7243 | 22031 | c_{S}a_{S}u_{S}08u_{S}u_{S}a_{S}g_{S}C_{S}C_{S}C_{S}u_{S}a_{S}g_{S}u_{S}C_{S}a_{S}C_{S}g_{S}g_{S}C_{S}u_{S}a | 15997 | Antisense |
| 268 | S27 | AUCUUAGCCCUAGUCACCGCUAG | 7244 | 22032 | a_{S}u_{S}C_{S}u_{S}u_{S}a_{S}g_{S}06C_{S}c_{S}u_{S}a_{S}g_{S}u_{S}c_{S}a_{S}C_{S}g_{S}g_{S}C_{S}u_{S}a_{S}g | 15998 | Antisense |
| 269 | S27 | UCUUAGCCCUAGUCACGGCUAGC | 7245 | 22033 | u_{S}c_{S}u_{S}u_{S}a_{S}g_{S}C_{S}c_{S}c_{S}u_{S}a_{S}g_{S}u_{S}C_{S}a_{S}c_{S}g_{S}g_{S}C_{S}u_{S}a_{S}g_{S}c | 15999 | Antisense |
| 270 | S27 | CUUAGCCCUAGUCACGGCUAGCU | 7246 | 22034 | C_{S}u_{S}u_{S}a_{S}g_{S}C_{S}C_{S}C_{S}u_{S}a_{S}g_{S}u_{S}C_{S}a_{S}C_{S}g_{S}g_{S}c_{S}u_{S}a_{S}g_{S}C_{S}u | 16000 | Antisense |
| 271 | S27 | UUAGCCCUAGUCACGGCUAGCUG | 7247 | 22035 | u_{S}u_{S}a_{S}g_{S}C_{S}C_{S}C_{S}u_{S}a_{S}g_{S}u_{S}C_{S}a_{S}C_{S}g_{S}g_{S}C_{S}u_{S}a_{S}g_{S}C_{S}u_{S}g | 16001 | Antisense |
| 272 | S27 | UAGCCCUAGUCACGGCUAGCUGU | 7248 | 22036 | u_{S}a_{S}g_{S}C_{S}C_{S}C_{S}u_{S}a_{S}g_{S}u_{S}C_{S}a_{S}C_{S}g_{S}g_{S}C_{S}u_{S}a_{S}g_{S}C_{S}u_{S}g_{S}u | 16002 | Antisense |
| 273 | S27 | AGCCCUAGUCACGGCUAGCUGUG | 7249 | 22037 | a_{S}g_{S}C_{S}C_{S}C_{S}u_{S}a_{S}g_{S}u_{S}C_{S}a_{S}C_{S}g_{S}g_{S}C_{S}u_{S}a_{S}g_{S}C_{S}u_{S}g_{S}u_{S}g | 16003 | Antisense |
| 274 | S27 | GCCCUAGUCACGGCUAGCUGUGA | 7250 | 22038 | g_{S}C_{S}C_{S}C_{S}u_{S}a 5g_{S}u_{S}C_{S}a_{S}C_{S}g_{S}g_{S}C_{S}u_{S}a_{S}g_{S}C_{S}u_{S}g_{S}u_{S}g_{S}a | 16004 | Antisense |
| 275 | S27 | CCCUAGUCACGGCUAGCUGUGAA | 7251 | 22039 | C_{S}C_{S}C_{S}u_{S}a_{S}g_{S}u_{S}C_{S}a_{S}C_{S}g_{S}g_{S}C_{S}u_{S}a_{S}g_{S}C_{S}u_{S}g_{S}u_{S}g_{S}a_{S}a | 16005 | Antisense |
| 276 | S27 | CCUAGUCACGGCUAGCUGUGAAA | 7252 | 22040 | C_{S}C_{S}u_{S}a_{S}g_{S}u_{S}C5a_{S}C_{S}g_{S}g_{S}C5u_{S}a_{S}g_{S}C5u_{S}g_{S}u_{S}g_{S}a_{S}a_{S}a | 16006 | Antisense |
| 277 | S27 | CUAGUCACGGCUAGCUGUGAAAG | 7253 | 22041 | C_{S}u_{S}a_{S}g_{S}u_{S}C_{S}a_{S}C_{S}g_{S}g_{S}C5u_{S}a_{S}g_{S}C_{S}u_{S}g_{S}u_{S}g_{S}a_{S}a_{S}a_{S}g | 16007 | Antisense |
| 278 | S27 | UAGUCACGGCUAGCUGUGAAAGG | 7254 | 22042 | u_{S}a_{S}g_{S}u_{S}C_{S}a_{S}C_{S}g_{S}g_{S}C_{S}u_{S}a_{S}g_{S}C_{S}u_{S}g_{S}u_{S}g_{S}a_{S}a_{S}a_{S}g_{S}g | 16008 | Antisense |
| 279 | S27 | AGUCACGGCUAGCUGUGAAAGGU | 7255 | 22043 | a_{S}g_{S}u_{S}C_{S}a_{S}C_{S}g_{S}g_{S}C5u_{S}a_{S}g_{S}C_{S}u_{S}g_{S}u_{S}g_{S}a_{S}a_{S}a_{S}g_{S}g_{S}u | 16009 | Antisense |
| 280 | S27 | GUCACGGCUAGCUGUGAAAGGUC | 7256 | 22044 | g_{S}u_{S}C_{S}a_{S}C_{S}g_{S}g_{S}C_{S}u_{S}a_{S}g_{S}C_{S}u_{S}g_{S}u_{S}g_{S}a_{S}a_{S}a_{S}g_{S}g_{S}u_{S}c | 16010 | Antisense |
| 281 | S27 | UCACGGCUAGCUGUGAAAGGUCC | 7257 | 22045 | u_{S}C_{S}a_{S}C_{S}g_{S}g_{S}C_{S}u_{S}a_{S}g_{S}C_{S}u_{S}g_{S}u_{S}g_{S}a_{S}a_{S}a_{S}g_{S}g_{S}u_{S}C_{S}c | 16011 | Antisense |
| 282 | S27 | CACGGCUAGCUGUGAAAGGUCCG | 7258 | 22046 | C_{S}a_{S}C_{S}g_{S}g_{S}C_{S}u_{S}a_{S}g_{S}C_{S}u_{S}g_{S}u_{S}g_{S}a_{S}a_{S}a_{S}g_{S}g_{S}u_{S}C_{S}C_{S}g | 16012 | Antisense |
| 283 | S27 | ACGGCUAGCUGUGAAAGGUCCGU | 7259 | 22047 | a_{S}C_{S}g_{S}g_{S}C_{S}u_{S}a_{S}g_{S}C_{S}u_{S}g_{S}u_{S}g_{S}a_{S}a_{S}a_{S}g_{S}g_{S}u_{S}C_{S}C_{S}g_{S}u | 16013 | Antisense |
| 284 | S27 | CGGCUAGCUGUGAAAGGUCCGUG | 7260 | 22048 | C_{S}g_{S}g_{S}C_{S}u_{S}a_{S}g_{S}C6u_{S}g_{S}u_{S}g_{S}a_{S}a_{S}a_{S}g_{S}g_{S}u_{S}C_{S}C_{S}g_{S}u_{S}g | 16014 | Antisense |
| 285 | S27 | GGCUAGCUGUGAAAGGUCCGUGA | 7261 | 22049 | g_{S}g_{S}C_{S}u_{S}a_{S}g_{S}C_{S}u_{S}g_{S}u_{S}g_{S}a_{S}a_{S}a_{S}g_{S}g_{S}u_{S}C_{S}C_{S}g_{S}u_{S}g_{S}a | 16015 | Antisense |
| 286 | S27 | GCUAGCUGUGAAAGGUCCGUGAG | 7262 | 22050 | g_{S}C_{S}u_{S}a_{S}g_{S}C_{S}u_{S}95u_{S}g_{S}a_{S}a_{S}a_{S}g_{S}g_{S}u_{S}C_{S}C_{S}g_{S}u_{S}g_{S}a_{S}g | 16016 | Antisense |
| 287 | S27 | CUAGCUGUGAAAGGUCCGUGAGC | 7263 | 22051 | C_{S}u_{S}a_{S}g_{S}c_{S}u_{S}g_{S}u_{S}g_{S}a_{S}a_{S}a_{S}g_{S}g_{S}u_{S}C_{S}C_{S}g_{S}u_{S}g_{S}a_{S}g_{S}c | 16017 | Antisense |
| 311 | S27 | GCAUGACUGCAGAGAGUGCUGAU | 7264 | 22052 | g_{S}C_{S}a_{S}u_{S}g_{S}a_{S}C_{S}u_{S}g_{S}C_{S}a_{S}g_{S}a_{S}g_{S}a_{S}g_{S}u_{S}g_{S}C_{S}u_{S}g_{S}a_{S}u | 16018 | Antisense |
| 312 | S27 | CAUGACUGCAGAGAGUGCUGAUA | 7265 | 22053 | C_{S}a_{S}u_{S}g_{S}a_{S}C_{S}u_{S}g_{S}05a_{S}95a_{S}g_{S}a_{S}g_{S}u_{S}g_{S}05u_{S}g_{S}a_{S}u_{S}a | 16019 | Antisense |
| 313 | S27 | AUGACUGCAGAGAGUGCUGAUAC | 7266 | 22054 | a_{S}u_{S}g_{S}a_{S}C_{S}u_{S}g_{S}C_{S}a_{S}g_{S}a_{S}g_{S}a_{S}g_{S}u_{S}g_{S}C_{S}u_{S}g_{S}a_{S}u_{S}a_{S}c | 16020 | Antisense |
| 314 | S27 | UGACUGCAGAGAGUGCUGAUACU | 7267 | 22055 | u_{S}g_{S}a_{S}e5u_{S}g_{S}C_{S}a_{S}g_{S}a_{S}g_{S}a_{S}g_{S}u_{S}g_{S}C_{S}u_{S}g_{S}a_{S}u_{S}a_{S}C_{S}u | 16021 | Antisense |
| 315 | S27 | GACUGCAGAGAGUGCUGAUACUG | 7268 | 22056 | g_{S}a_{S}C_{S}u_{S}g_{S}C_{S}a_{S}g_{S}a_{S}g_{S}a_{S}g_{S}u_{S}g_{S}C_{S}u_{S}g_{S}a_{S}u_{S}a_{S}C_{S}u_{S}g | 16022 | Antisense |
| 316 | S27 | ACUGCAGAGAGUGCUGAUACUGG | 7269 | 22057 | a_{S}C_{S}u_{S}g_{S}C_{S}a_{S}g_{S}a_{S}g_{S}a_{S}g_{S}u_{S}g_{S}C_{S}u_{S}g_{S}a_{S}u_{S}a_{S}C_{S}u_{S}g_{S}g | 16023 | Antisense |
| 317 | S27 | CUGCAGAGAGUGCUGAUACUGGC | 7270 | 22058 | C_{S}u_{S}g_{S}C_{S}a_{S}g_{S}a_{S}g_{S}a_{S}g_{S}u_{S}g_{S}C_{S}u_{S}g_{S}a_{S}u_{S}a_{S}C_{S}u_{S}g_{S}g_{S}c | 16024 | Antisense |
| 318 | S27 | UGCAGAGAGUGCUGAUACUGGCC | 7271 | 22059 | u_{S}g_{S}C_{S}a_{S}g_{S}a_{S}g_{S}a_{S}g_{S}u_{S}g_{S}C_{S}u_{S}g_{S}a_{S}u_{S}a_{S}C_{S}u_{S}g_{S}g_{S}C_{S}c | 16025 | Antisense |
| 319 | S27 | GCAGAGAGUGCUGAUACUGGCCU | 727 | 222060 | g_{S}C_{S}a_{S}g_{S}a_{S}g_{S}a_{S}g_{S}u_{S}g_{S}C_{S}u_{S}g_{S}a_{S}u_{S}a_{S}C_{S}u_{S}g_{S}g_{S}C_{S}C_{S}u | 16026 | Antisense |
| 320 | S27 | CAGAGAGUGCUGAUACUGGCCUC | 7273 | 22061 | c_{S}a_{S}g_{S}a_{S}g_{S}a_{S}g_{S}u_{S}g_{S}C_{S}u_{S}g_{S}a_{S}u_{S}a_{S}C_{S}u_{S}g_{S}g_{S}C_{S}C_{S}u_{S}c | 16027 | Antisense |
| 321 | S27 | AGAGAGUGCUGAUACUGGCCUCU | 7274 | 22062 | a_{S}g_{S}a_{S}g_{S}a_{S}95u_{S}g_{S}C_{S}u_{S}g_{S}a_{S}u_{S}a_{S}C_{S}u_{S}g_{S}g_{S}C_{S}C_{S}u_{S}C_{S}u | 16028 | Antisense |
| 322 | S27 | GAGAGUGCUGAUACUGGCCUCUC | 7275 | 22063 | g_{S}a_{S}g_{S}a_{S}g_{S}u_{S}g_{S}C_{S}u_{S}g_{S}a_{S}u_{S}a_{S}C_{S}u_{S}g_{S}g_{S}C_{S}C_{S}u_{S}c_{S}u_{S}c | 16029 | Antisense |
| 157 | HCV+ | CGGAACCGGUGAG | 7276 | 22524 | c_{S}u_{S}C_{S}a_{S}cc cUGAuGaggccguuaggccGaa Iuuccg B | 16030 | Inozyme |
| 167 | HCV+ | GAGUACACCGGAA | 7277 | 22525 | u_{S}u_{S}C_{S}C_{S}gg cuGAuGaggccguuaggccGaa luacuc B | 16031 | Inozyme |
| 139 | HCV+ | GAGAGCCAUAGUG | 7278 | 22526 | c_{S}a_{S}c_{S}u_{S}aucTGAuGaggccguuaggccGaa Icucuc B | 16032 | Inozyme |
| 140 | HCV+ | AGAGCCAUAGUGG | 7279 | 22527 | c_{S}c_{S}a_{S}c_{S}uacuGAuGaggccguuaggccGaa Igcucu B | 16033 | Inozyme |
| 281 | HCV+ | AAGGCCUUGUGGU | 7280 | 22528 | a_{S}c_{S}c_{S}a_{S}cacUGAuGaggccguuaggccGaa Igccuu B | 16034 | Inozyme |
| 130 | HCV+ | CCCUCCCGGGAGA | 7281 | 22529 | u_{S}c_{S}u_{S}c_{S}cccUGAuGaggccguuaggccGaa Igaggg B | 16035 | Inozyme |
| 280 | HCV+ | AAAGGCCUUGUGG | 728 | 222530 | c_{S}c_{S}a_{S}c_{S}aacuGAuGaggccguuaggccGaa Iccuuu B | 16036 | Inozyme |
| 149 | HCV+ | GUGGUCUGCGGAA | 7283 | 22531 | u_{S}u_{S}C_{S}c_{S}gccUGAuGaggccguuaggccGaa laccac B | 16037 | Inozyme |
| 194 | HCV+ | GGGUCCUUUCUUG | 7284 | 22532 | c_{S}a_{S}a_{S}g_{S}aacUGAuGaggccguuaggccGaa Igaccc B | 16038 | Inozyme |
| 255 | HCV+ | GCUAGCCGAGUAG | 7285 | 22533 | c_{S}u_{S}a_{S}c_{S}uccUGAuGaggccguuaggccGaa Icuago B | 16039 | Inozyme |
| 294 | HCV+ | ACUGCCUGAUAGG | 7286 | 22534 | c_{S}c_{S}u_{S}a_{S}ucctlGAuGaggccguuaggccGaa Igcagu B | 16040 | Inozyme |
| 293 | HCV+ | UACUGCCUGAUAG | 7287 | 22535 | c_{S}u_{S}a_{S}u_{S}cacuGAuGaggccguuaggccGaa Icagua B | 16041 | Inozyme |
| 290 | HCV+ | UGGUACUGCCUGA | 7288 | 22536 | u_{S}c_{S}a_{S}g_{S}gccUGAuGaggccguuaggccGaa luacca B | 16042 | Inozyme |
| 169 | HCV+ | GUACACCGGAAUU | 7289 | 22537 | a_{S}a_{S}u_{S}u_{S}cccUGAuGaggccguuaggccGaa luguac B | 16043 | Inozyme |
| 293 | HCV+ | GUACUGCCUGAUAGG | 7290 | 22544 | c_{S}c_{S}u_{S}a_{S}ucacTlGAuGaggccguuaggccGaa Icaguac B | 16044 | Inozyme |
| 294 | HCV+ | UACUGCCUCAUAGGG | 7291 | 22545 | c_{S}c_{S}c_{S}u_{S}auccUGAuGaggccguuaggccGaa Igcagua B | 16045 | Inozyme |
| 281 | HCV+ | AAAGGCCUUGUGGUA | 729 | 222546 | u_{S}a_{S}c_{S}c_{S}acacUGAuGaggccguuaggccGaa Igccuuu B | 16046 | Inozyme |
| 166 | HCV+ | UGAGUACACCGGA | 7293 | 22549 | u_{S}c_{S}c_{S}g_{S}gucUGAUGaggccguuaggccGaa Uacuca B | 16047 | Amberzyme |
| 168 | HCV+ | AGUACACCGGAAU | 7294 | 22550 | a_{S}u_{S}u_{S}c_{S}cgcUGAUGaggccguuaggccGaa Uguacu B | 16048 | Amberzyme |
| 141 | HCV+ | GAGCCAUAGUGGU | 7295 | 22551 | a_{S}c_{S}c_{S}a_{S}cucUGAUGaggccguuaggccGaa Uggcuc B | 16049 | Amberzyme |
| 156 | HCV+ | GCGGAACCGGUGA | 7296 | 22552 | u_{S}c_{S}a_{S}C_{S}cgcuGAUGaggccguuaggccGaa Uuccgc B | 16050 | Amberzyme |
| 155 | HCV+ | UGCGGAACCGGUG | 7297 | 22553 | c_{S}a_{S}C_{S}c_{S}ggcUGAuGaggccguuaggccGaa Uccgca B | 16051 | Amberzyme |
| 289 | HCV+ | GUGGUACUGCCUG | 7298 | 22554 | c_{S}a_{S}g_{S}g_{S}cacUGAtlGaggccguuaggccGaa Uaccac B | 16052 | Arnberzyme |
| 297 | HCV+ | GCCUGAUAGGGUG | 7299 | 22555 | c_{S}a_{S}c_{S}c_{S}cucuGAUGaggccguuaggccGaa Ucaggc B | 16053 | Amberzyme |
| 166 | HCV+ | GUGAGUACACCGGAA | 7300 | 22556 | u_{S}u_{S}c_{S}C_{S}ggucuGAuGaggccguuaggccGaa Uacucac B | 16054 | Amberzyme |
| 141 | HCV+ | AGAGCCAUAGUGGUC | 7301 | 22557 | g_{S}a_{S}C_{S}c_{S}acucUGAUGaggccguuaggccGaa Uggcucu B | 16055 | Amberzyine |
| 156 | HCV+ | UGCGGAACCGGUGAG | 730 | 222558 | c_{S}u_{S}c_{S}a_{S}ccgcUGAUGaggccguuaggccGaa Uuccgca B | 16056 | Amberzyme |
| 155 | HCV+ | CUGCGGAACCGGUGA | 7303 | 22559 | u_{S}c_{S}a_{S}c_{S}cggcUGAUGaggccguuaggccGaa Uccgcag B | 16057 | Amberzyme |
| 289 | HCV+ | UGUGGUACUGCCUGA | 7304 | 22560 | u_{S}c_{S}a_{S}g_{S}gcacUGAUGaggccguuaggccGaa Uaccaca B | 16058 | Amberzyme |
| 297 | HCV+ | UGCCUGAUAGGGUGC | 7305 | 22561 | g_{S}c_{S}a_{S}c_{S}ccucUGAUGaggccguuaggccGaa Ucaggca B | 16059 | Amberzyme |
| 168 | HCV+ | GAGUACACCGGAAUU | 7306 | 22562 | a_{S}agu_{S}u_{S}ccgcUGAuGaggccguuaggccGaa Uguacuc B | 16060 | Amberzyme |
| 166 | HCV− | UCCGGUGUACUCA | 7307 | 22563 | u_{S}g_{S}a_{S}g_{S}uagccgaaaggCgagugaGguCu accgga B | 16061 | Zinzyme |
| 168 | HCV− | ATCCGCUGUACU | 7308 | 22564 | a_{S}g_{S}u_{S}a_{S}cagccgaaaggCgagugaGguCu cggaau B | 16062 | Zinzyme |
| 138 | HCV− | ACUAUGGCUCUCC | 7309 | 22565 | g_{S}g_{S}a_{S}g_{S}aggccgaaaggCgagugaGguCu cauagu B | 16063 | Zinzyme |
| 156 | HCV− | UCACCGGUUCCGC | 7310 | 22566 | g_{S}c_{S}g_{S}g_{S}aagccgaaaggCgagugaGgucu cgguga B | 16064 | Zinzyme |
| 236 | HCV− | GCGGGGGCACGCC | 7311 | 22567 | g_{S}g_{S}c_{S}g_{S}uggccgaaaggCgagugaGguCu ccccgc B | 16065 | Zinzyme |
| 279 | HCV− | CACAAGGCCUUUC | 731 | 222568 | g_{S}a_{S}a_{S}a_{S}gggccgaaaggCgagugaGguCu cuugug B | 16066 | Zinzyme |
| 151 | HCV− | GGUUCCGCAGACC | 7313 | 22569 | g_{S}g_{S}u_{S}c_{S}uggccgaaaggCgagugaGguCu ggaacc B | 16067 | Zinzyme |
| 292 | HCV− | UAUCAGGCAGUAC | 7314 | 22570 | g_{S}u_{S}a_{S}c_{S}uggccgaaaggCgagugaGguCu cugaua B | 16068 | Zinzyme |
| 289 | HCV− | CAGGCAGUACCAC | 7315 | 22571 | g_{S}u_{S}g_{S}g_{S}uagccgaaaggCgagugaGguCu ugccug B | 16069 | Zinzyme |
| 166 | HCV− | UUCCGGUGUACUCAC | 7316 | 22572 | g_{S}u_{S}g_{S}a_{S}guagccgaaaggCgagugaGguCu accggaa B | 16070 | Zinzyme |
| 279 | HCV− | CCACAAGGCCUUUCG | 7317 | 22573 | c_{S}g_{S}a_{S}a_{S}agggccgaaaggCgagugaGguCu cuugugg B | 16071 | Zinzyme |
| 156 | HCV− | CUCACCGGUUCCGCA | 7318 | 22574 | u_{S}g_{S}c_{S}g_{S}gaagccgaaag_{S}CgagugaGguCu cggugag B | 16072 | Zinzyme |
| 138 | HCV− | CACUAUGGCUCUCCC | 7319 | 22575 | g_{S}g_{S}g_{S}a_{S}gaggccgaaaggCgagugaGgucu cauagug B | 16073 | Zinzyme |
| 151 | HCV− | CGGUUCCGCAGACCA | 7320 | 22576 | u_{S}g_{S}g_{S}u_{S}cuggccgaaaggCgagugaGguCu ggaaccg B | 16074 | Zinzyme |
| 292 | HCV− | CUAUCAGGCAGUACC | 7321 | 22577 | g_{S}g_{S}u_{S}a_{S}cuggccgaaaggCgagugaGguCu cugauag B | 16075 | Zinzyme |
| 289 | HCV− | UCAGGCAGUACCACA | 7322 | 22578 | u_{S}g_{S}u_{S}g_{S}guagccgaaaggCgagugaGguCu ugccuga B | 16076 | Zinzyme |
| 168 | HCV− | AAUUCCGGUGUACUC | 7323 | 22579 | g_{S}a_{S}g_{S}u_{S}acagccgaaaggCgagugaGguCu cggaauu B | 16077 | Zinzyme |
| 163 | HCV− | GGUGUACUCACCG | 7324 | 22580 | C_{S}g_{S}g_{S}u_{S}gacuGAUGaggccguuaggccGaa Uacacc B | 16078 | Amberzyme |
| 159 | HCV− | UACUCACCGGUUC | 7325 | 22581 | g_{S}a_{S}a_{S}C_{S}cgcuGAUGaggccguuaggccGaa Ugagua B | 16079 | Amberzyme |
| 140 | HCV− | CCACUAUGGCUCU | 7326 | 22582 | a_{S}g_{S}a_{S}g_{S}cccuGAUGaggccguuaggccGaa Uagugg B | 16080 | Arnberzyme |
| 281 | HCV− | ACCACAAGGCCUU | 7327 | 22583 | a_{S}a_{S}g_{S}g_{S}cccUGAUGaggccguuaggccGaa Uguggu B | 16081 | Amberzyme |
| 233 | HCV− | GGGGCACGCCCAA | 7328 | 22584 | u_{S}u_{S}g_{S}g_{S}gccuGAUGaggccguuaggccGaa Ugcccc B | 16082 | Amberzyrne |
| 143 | HCV− | AGACCACUAUGGC | 7329 | 22585 | g_{S}c_{S}c_{S}a_{S}uacuGAUGaggccguuaggccGaa Uggucu B | 16083 | Arnberzyrne |
| 146 | HCV− | CGCAGACCACUAU | 7330 | 22586 | a_{S}u_{S}a_{S}g_{S}ugcUGAUGaggccguuaggccGaa Ucugcg B | 16084 | Amberzyme |
| 195 | HCV− | CCAAGAAAGGACC | 7331 | 22587 | g_{S}g_{S}u_{S}c_{S}cucUGAuGaggccguuaggccGaa Ucuugg B | 16085 | Amberzyme |
| 194 | HCV− | CAAGAAAGGACCC | 733 | 222588 | g_{S}g_{S}g_{S}u_{S}cccUGAUGaggccguuaggccGaa Uucuug B | 16086 | Amberzyme |
| 283 | HCV− | GUACCACAAGGCC | 7333 | 22589 | g_{S}g_{S}C_{S}C_{S}uucUGAUGaggccguuaggccGaa Ugguac B | 16087 | Atnberzyme |
| 286 | HCV− | GCAGUACCACAAG | 7334 | 22590 | cgu_{S}u_{S}ggugcUGAuGaggccguuaggccGaa Uacugc B | 16088 | Amberzyme |
| 296 | HCV− | ACCCUAUCAGGCA | 7335 | 22591 | u_{S}g_{S}c_{S}c_{S}ugcuCAuGaggccguuaggccGaa Uagggu B | 16089 | Amberzyme |
| 190 | HCV− | AAAGGACCCGGUC | 7336 | 22592 | g_{S}a_{S}c_{S}c_{S}ggcUGAUGaggccguuaggccGaa Uccuuu B | 16090 | Amberzyme |
| 163 | HCV− | CGGUGUACUCACCGC | 7337 | 22593 | C_{S}c_{S}g_{S}g_{S}ugacUGAUCaggccguuaggccCaa Uacaccg B | 16091 | Amberzyme |
| 140 | HCV− | ACCACUAUGGCUCUC | 7338 | 22594 | g_{S}a_{S}g_{S}a_{S}gcccUGAUGaggccguuaggccGaa Uaguggu B | 16092 | Amberzyme |
| 159 | HCV− | GUACUCACCGGUUCC | 7339 | 22595 | g_{S}g_{S}a_{S}a_{S}ccgcuGAUGaggccguuaggccGaa Ugaguac B | 16093 | Amberzyme |
| 233 | HCV− | GGGGGCACGCCCAAA | 7340 | 22596 | u_{S}u_{S}u_{S}g_{S}ggccUGAUGaggccguuaggccGaa Ugccccc B | 16094 | Amberzyme |
| 143 | HCV− | CAGACCACUAUGCCU | 7341 | 22597 | a_{S}g_{S}c_{S}c_{S}auacUGAUcaggccguuaggccCaa Uggucug B | 16095 | Amberzyme |
| 146 | HCV− | CCGCAGACCACUAUG | 734 | 222598 | c_{S}a_{S}u_{S}a_{S}gugcUGAUGaggccguuaggccGaa Ucugcgg B | 16096 | Arnberzyme |
| 195 | HCV− | UCCAAGAAAGGACCC | 7343 | 22599 | g_{S}g_{S}g_{S}u_{S}ccucuGAUGaggccguuaggccGaa Ucuugga B | 16097 | Amberzyme |
| 283 | HCV− | AGUACCACAAGGCCU | 7344 | 22600 | a_{S}g_{S}g_{S}C_{S}cuucUGAUGaggccguuaggccGaa Ugguacu B | 16098 | Amberzyme |
| 281 | HCV− | UACCACAAGCCCUUU | 7345 | 22601 | a_{S}a_{S}a_{S}g_{S}gcccUGAUGaggccguuaggccGaa Uguggua B | 16099 | Arnberzyme |
| 296 | HCV− | CACCCUAUCAGGCAG | 7346 | 22602 | c_{S}u_{S}g_{S}c_{S}cugcUGAuGaggccguuaggccGaa Uagggug B | 16100 | Amberzyme |
| 286 | HCV− | GGCAGUACCACAAGG | 7347 | 22603 | c_{S}c_{S}u_{S}u_{S}gugcUGAUGaggccguuaggccGaa Uacugcc B | 16101 | Amberzyme |
| 7985 | HCV− | UCUCAGU G UCUUCCA | 7348 | 22719 | uggaaga uGAUg gcauGcacuaugc gCg acugaga B | 16102 | G-cleaver |
| 4832 | HCV− | UGUAUAU G CCUCUCC | 7349 | 22720 | ggagagg uGAUg gcauGcacuaugc gCg auauaca B | 16103 | G-cleaver |
| 4153 | HCV− | ACCGUGU G CCUUAGA | 7350 | 22721 | ucuaagg uGAUg gcauGcacuaugc gCg acacggu B | 16104 | 0-cleaver |
| 3200 | HCV− | GUGGAGU G AGGUGGU | 7351 | 22722 | accaccu uGAUg gcauGcacuaugc gCg acuccac B | 16105 | 0-cleaver |
| 1682 | HCV− | ACGAGUU G AACCUGU | 7352 | 22723 | acagguu uGAUg gcauocacuaugc gCg aacucgu B | 16106 | 0-cleaver |
| 896 | HCV+ | CCUGUCU G ACCAUCC | 7353 | 22724 | ggauggu uGAUg gcauocacuaugc gCg agacagg B | 16107 | 0-cleaver |
| 2504 | HCV+ | UCCUGUU G CUUUUCC | 7354 | 22725 | ggaaaag UGAUg gcauGcacuaugc gCg aacagga B | 16108 | 0-cleaver |
| 2651 | HCV+ | UCCUCGU G UUCUUCU | 7355 | 22726 | agaagaa uGAUg gcauGcacuaugc gCg acgagga B | 16109 | 0-cleaver |
| 4094 | HCV+ | ACAAAGU G CUCGUCC | 7356 | 22727 | ggacgag uGAUg gcauGcacuaugc gCg acuuugu B | 16110 | G-cleaver |
| 8970 | HCV+ | GCCACUU G ACCUACC | 7357 | 22728 | gguaggu uGAUg gcauGcacuaugc gCg aaguggc B | 16111 | 0-cleaver |
| 1200 | HCV+ | CUUCCUC G UCUCUCA | 7358 | 22747 | ugagaga gccgaaaggCgagugaGGuCu gaggaag B | 16112 | Zinzyme |
| 1211 | HCV+ | CUCAOCU G UUCACCU | 7359 | 22748 | aggugaa gccgaaaggCgaguga0GuCu agcugag B | 16113 | Zinzyme |
| 2504 | HCV+ | UCCUGUU G CUUUUCC | 7354 | 22749 | ggaaaag gccgaaaggCgagugaGGuCu aacagga B | 16114 | Zinzyme |
| 2651 | HCV+ | UCCUCGU G UUCUUCU | 7355 | 22750 | agaagaa gccgaaaggCgagugaoGuCu acgagga B | 16115 | Zinzyme |
| 8811 | HCV+ | CACUCCA G UCAACUC | 7360 | 22751 | gaguuga gccgaaaggCgagugaoouCu uggagug B | 16116 | Zinzyme |
| 8594 | HCV− | UCOCCGC G UCCUCUU | 7361 | 22752 | aagagga gccgaaaggCgagugaoouCu gcggcga B | 16117 | Zinzyme |
| 7985 | HCV− | UCUCAGU G UCUUCCA | 7348 | 22753 | uggaaga gccgaaaggCgagugaoouCu acugaga B | 16118 | Zinzyme |
| 6611 | HCV− | CCUCCAC G UACUCCU | 7362 | 22754 | aggagua gccgaaaggCgagugaGGuCu guggagg B | 16119 | Zinzyme |
| 5633 | HCV− | UCCACAU G UGCUUCG | 7363 | 22755 | cgaagca gccgaaaggCgagugaoouCu augugga B | 16120 | Zinzyme |
| 821 | HCV− | UCACGCC G UCUUCCA | 7364 | 22756 | uggaaga gccgaaaggCgagugagguCu ggcguga B | 16121 | Zinzyme |
| 870 | HCV+ | CUCUAUC U UCCUCUU | 7365 | 22775 | aagagga CUgAUgAggccguuaggccgAA Iauagag B | 16122 | Inozyme |
| 1210 | HCV+ | UCUCAGC U GUUCACC | 7366 | 22776 | ggugaac CUgAUgAggccguuaggccgAA Icugaga B | 16123 | Inozyme |
| 2642 | HCV+ | UCCUCUC C UUCCUCG | 7367 | 22777 | cgaggaa CUgAUgAggccguuaggccgAA lagagga B | 16124 | Inozyme |
| 5726 | HCV+ | UCACAGC C UCCAUCA | 7368 | 22778 | ugaugga CUgAUgAggccguuaggccgAA Icuguga B | 16125 | Inozyme |
| 8142 | HCV+ | CUCCACC C UUCCUCA | 7369 | 22779 | ugaggaa CUgAUgAggccguuaggccgAA Iguggag B | 16126 | Inozyme |
| 7990 | HCV− | UGGUGUC U CAGUGUC | 7370 | 22780 | gacacug CUgAUgAggccguuaggccgAA lacacca B | 16127 | Inozyme |
| 7813 | HCV− | CUUCGCC U UCAUCUC | 7371 | 22781 | gagauga CUgAUgAggccguuaggccgAA Igcgaag B | 16128 | Inozyme |
| 7137 | HCV− | ACCUCUC U CUCAUCC | 7372 | 22782 | ggaugag CUgAUgAggccguuaggccgAA Iagaggu B | 16129 | Inozyme |
| 6084 | HCV− | UUCAUCC A CUOCACA | 7373 | 22783 | ugugcag CUgAUgAggccguuaggccgAA Igaugaa B | 16130 | Inozyme |
| 2554 | HCV− | CAACAGC A UCAUCCA | 7374 | 22784 | uggauga CUgAUgAggccguuaggccgAA Icuguug B | 16131 | Inozyme |
| 1202 | HCV+ | UCCUCGU C UCUCAAC | 7375 | 22943 | gcugaga CUgAUgAggccguuaggccgAA Acgagga B | 16132 | Hammerhead |
| 1607 | HCV− | CGCACAU U AACAGGA | 7376 | 22944 | uccuguu CU0AUGAggccguuaggccgAA Augugcc B | 16133 | Hammerhead |
| 2639 | HCV+ | CCAUCCU C UCCUUCC | 7377 | 22945 | ggaagga CUgAUgAggccguuaggccgAA Aggaugc B | 16134 | Hammerhead |
| 6610 | HCV+ | GAGGAGU A COUGGAG | 7378 | 22946 | cuccacg CUgAUgAggccguuaggccgAA Acuccuc B | 16135 | Hammerhead |
| 9014 | HCV+ | GCGCAUU U UCACUCC | 7379 | 22947 | ggaguga CUgAUgAggccguuaggccgAA Aaugcgc B | 16136 | Hammerhead |
| 8605 | HCV− | GACUCGU A GGCUCGC | 7380 | 22948 | gcgagcc CUGAUGAggccguuaggccgAA Acgaguc B | 16137 | Hammerhead |
| 7983 | HCV− | UCAGUGU C UUCCAGC | 7381 | 22949 | gcuggaa CUgAUgAggccguuaggccgAA Acacuga B | 16138 | Hammerhead |
| 7136 | HCV− | CCUCUCU C UCAUCCU | 7382 | 22950 | aggauga CUgAUgAggccguuaggccgAA Agagagg B | 16139 | Hammerhead |
| 6609 | HCV− | UCCACGU A CUCCUCA | 7383 | 22951 | ugaggag CUgAUgAggccguuaggccGAA Acgugga B | 16140 | Hammerhead |
| 6292 | HCV− | CGUGCAU A UCCAGUC | 7384 | 22952 | gacugga CUgAUgAggccguuaggccgAA Augcacg B | 16141 | Hammerhead |
| 867 | HCV+ | UUUCUCU A UCUUCCU | 7385 | 22971 | aggaaga GGCTAGCTACAACGA agagaaa B | 16142 | DNAzyme |
| 1200 | HCV+ | CUUCCUC G UCUCUCA | 7358 | 22972 | ugagaga GGCTAGCTACAACGA gaggaag B | 16143 | DNAzyme |
| 1211 | HCV+ | CUCAOCU G UUCACCU | 7359 | 22973 | aggugaa GGCTAGCTACAACGA agcugag B | 16144 | DNAzyme |
| 5730 | HCV+ | AGCCUCC A UCACCAG | 7386 | 22974 | cugguga GGCTAGCTACAACGA ggaggcu B | 16145 | DNAzyme |
| 6533 | HCV+ | UCAACGC A UACACCA | 7387 | 22975 | uggugua GGCTAGCTACAACGA gcguuga B | 16146 | DNAzyme |
| 8594 | HCV− | UCGCCGC G UCCUCUU | 7361 | 22976 | aagagga GGCTAGCTACAACGA gcggcga B | 16147 | DNAzyme |
| 7810 | HCV− | CGCCUUC A UCUCCUU | 7388 | 22977 | aaggaga GGCTAGCTACAACGA gaaggcg B | 16148 | DNAzyme |
| 7133 | HCV− | CUCUCUC A UCCUCCU | 7389 | 22978 | aggagga GGCTAGCTACAACGA gagagag B | 16149 | DNAzyme |
| 6611 | HCV− | CCUCCAC G UACUCCU | 736 | 222979 | aggagua GGCTAGCTACAACGA guggagg B | 16150 | DNAzyme |
| 2300 | HCV− | CCUCCAA A UCACAAC | 7390 | 22980 | guuguga GGCTAGCTACAACGA uuggagg B | 16151 | DNAzyme |
| 195 | HCV+ | GGGUCCU U UCUUGGA | 7148 | 23072 | c_{S}c_{S}a_{S}a_{S}ga cUGAuGaggcgWWagccGaa Aggacc B | 16152 | Hammerhead |
| 195 | HCV+ | GGGUCCU U UCUUGGA | 7148 | 23076 | WWWWWc_{S}c_{S}a_{S}a_{S}ga cUGAuGaggcguuagccGaa Aggacc B | 16153 | Hammerhead |
| 195 | HCV+ | GGGUCCU U UCUUGGA | 7148 | 23077 | WWWc_{S}c_{S}a_{S}a_{S}ga cUGAuGaggcgWWwagccGaa Aggacg B | 16154 | Hammerhead |
| 195 | HCV+ | GGGUCCU U UCUUGGA | 7148 | 23086 | c_{S}c_{S}a_{S}a_{S}ga cuGAuGaggcgWWWagccGaa Aggacc B | 16155 | Hammerhead |

lower ca_{S}e=2+-O-methyl
UPPER CASE=RIBO
B=inverted deoxy abasic
U=2′-deoxy-2′-aming Uridine
C=2′-deoxy-2′-aming Cytidine
U=2′-deoxy-2′-aming Uridine
Z=BRdU (5-brgmg-2′-deoxy Uridine)
W=acyclic galactgse-amine linker
UNDERLINE=deoxy nuclegtide

TABLE XXI

ANTI HCV AMINO CONTAINING HAMMERHEAD RIBOZYME
AND CONTROL SEQUENCES

| | | | | |
|---|---|---|---|---|
| | | HCV 5′UTR | | Rz Seq |
| pos | RPI# | Site | Core | ID |

| | | | | | |
|---|---|---|---|---|---|
| | | | Ribozyme Sequences (5′-3′) | | |
| 62 | 12257 | HCV-62 | g_{S}c_{S}g_{S}ugaa cUGAUGaggccguuaggccGaa AcaguagB | Active | 15897 |
| 79 | 12258 | HCV-79 | a_{S}u_{S}g_{S}gcua cUGAUGaggccguuaggccGaa AcgcuuuB | Active | 15898 |
| 81 | 12249 | HCV-81 | c_{S}C_{S}a_{S}uggc cUGAUGaggccguuaggccGaa AgacgcuB | Active | 15899 |
| 104 | 12259 | HCV-104 | g_{S}c_{S}u_{S}gcac cUGAUGaggccguuaggccGaa AcacucaB | Active | 15900 |
| 142 | 12250 | HCV-142 | a_{S}g_{S}a_{S}ccac cUGAUGaggccguuaggccGaa AuggcucB | Active | 15901 |
| 148 | 12251 | HCV-148 | u_{S}u9c_{S}cgca cUGAUGaggccguuaggccGaa AccacuaB | Active | 15902 |
| 165 | 12260 | HCV-165 | u_{S}C_{S}c_{S}ggug cUGAUGaggccguuaggccGaa AcucaccB | Active | 15903 |
| 192 | 12261 | HCV-192 | a_{S}a_{S}g_{S}aaag cUGAuGaggccguuaggccGaa AcccgguB | Active | 15904 |
| 195 | 12252 | HCV-195 | u_{S}c_{S}c_{S}aaga cUGAUGaggccguuaggccGaa AggacccB | Active | 15905 |
| 196 | 12262 | HCV-196 | a_{S}ugC_{S}caag cUGAUGaggccguuaggccGaa AaggaccB | Active | 15906 |
| 270 | 12263 | HCV-270 | c_{S}u_{S}u_{S}ucgc cuGAUGaggccguuaggccGaa AcccaacB | Active | 15907 |
| 282 | 12264 | HCV-282 | g_{S}u_{S}a_{S}ccac cUGAUGaggccguuaggccGaa AggccuuB | Active | 15908 |
| 306 | 12265 | HCV-306 | c_{S}a_{S}C_{S}ucgc cUGAuGaggccguua9gccGaa AgcacccB | Active | 15909 |
| 325 | 12253 | HCV-325 | u_{S}c_{S}u_{S}acga cUGAUGaggccguuaggccGaa AccucccB | Active | 15910 |
| 330 | 12254 | HCV-330 | c_{S}a_{S}c_{S}gguc cUGAUGaggccguuaggccGaa AcgagacB | Active | 15911 |
| | | | Control Sequences | | |
| 79 | 13274 | HCV-79AC2 | c_{S}u_{S}u_{S}aggu cUAGUGaggccguuaggccGau AguucucB | Attenuated | 16171 |
| 81 | 13271 | HCV-81AC | u_{S}c_{S}u_{S}gccg cuAGuGaggccguuaggccGau AgugaccB | Attenuated | 16172 |
| 142 | 13270 | HCV-142AC | a_{S}a_{S}c_{S}ccug cUAGUGaggccguuaggccGau AgcucguB | Attenuated | 16173 |
| 192 | 13272 | HCV-192AC | a_{S}g_{S}u_{S}agaa cUAGUGaggccguuaggccGau AgcugccB | Attenuated | 16174 |
| 195 | 13269 | HCV-195AC | gsa_{S}u_{S}ucca cUAGUcaggccguuaggccGau Acgcgac_{S} | Attenuated | 16175 |
| 282 | 13273 | HCV-282AC | g_{S}c_{S}c_{S}auuc cUAGuGaggccguuaggccGau AucuggcB | Attenuated | 16176 |
| 330 | 13268 | 14CV-330AC | c_{S}c_{S}a_{S}ggcu cUAGUGaggccguuaggccGau AaugcgcB | Attenuated | 16177 |
| 195 | 15291 | HCV-195BAC3 | u_{S}c_{S}c_{S}aaga cUAGUGacgccguuaggcgGaa AggacccB | Attenuated | 16178 |
| 195 | 15292 | HCV-195SAC3 | a_{S}g_{S}a_{S}cuac cUAGuGacgccguuaggcgGaa AcccgagS | Attenuated | 16179 |
| 330 | 15294 | HCV-330BAC | c_{S}a_{S}C_{S}gguc cUAGt7GacgccguuaggcgGaa AcgagacB | Attenuated | 16180 |
| 330 | 15295 | HCV-330SAC | g_{S}c_{S}u_{S}ccga cUAGUGacgccguuaggcgGaa AgacacgB | Attenuated | 16181 |

UPPER CASE = RIBO;
lower case = 2′-O-methyl;
B = inverted deoxyabasic;
s = phosphorothioate linkage
U = 2′-deoxy-2′-amino uridine

TABLE XXII

ANTI HCV SITE 330 ANTISENSE NUCLEIC ACID AND
SCRAMBLED CONTROL SEQUENCES

| | | | |
|---|---|---|---|
| pos | RPI# | Alias | Seq ID # |

| | | | | |
|---|---|---|---|---|
| | | | Antisense Nucleic Acid | |
| 330 | 17501 | HCV.5-330 | GₛTₛGₛC_{S}TₛC_{S}A_{S}TₛGₛA_{S}TₛGₛC_{S}A_{S}C_{S}G_{S}GₛTₛC_{S}T | 15898 |
| | | antisense | | |
| 330 | 17498 | HCV.5-330 | G_{S}T_{S}G_{S}C_{S}T_{S}C_{S}A6T8G_{S}G_{S}T_{S}G5 C_{S}A_{S}C_{S}G_{S}G_{S}T_{S}C_{S}T | 16182 |
| | | antisense | | |
| | | | Control Sequence | |
| 330 | 17499 | HCV.5-330 | T_{S}G_{S}A_{S}TₛC_{S}A_{S}G_{S}G_{S}T_{S}C_{S}T_{S}G_{S}C_{S}T_{S}G_{S}C_{S}G_{S}T_{S}GₛC | 16183 |
| | | scrambled | | |
| 330 | 17502 | HCV.5-330 | T_{S}G_{S}A_{S}T_{S}C_{S}A_{S}G_{S}G_{S}T_{S}C_{S}T_{S}G_{S}C_{S}T_{S}G_{S}C_{S}A_{S}T_{S}G_{S}C | 16184 |
| | | Scrambled | | |

UPPER CASE = Deoxy Nucleotide
s = phosphorothioate

TABLE XXIII

| IN VITRO CLEAVAGE DATA, ANTI-HCV ENZYMATIC NUCLEIC ACIDS | | | | | | | | |
|---|---|---|---|---|---|---|---|---|
| | | | | | % Substrate | | | |
| Seq ID # | RPI# | Motif | Site (+/−) | Enzymatic Nucleic Acid Sequence | Cleaved in 3 hours | Substrate Sequence | Seq ID # | Substrate RPI# |

| | | | | | | | | |
|---|---|---|---|---|---|---|---|---|
| 16132 | 22943 | Hammerhead | 1190 (+) | gcugaga CUGAUGAggccguuaggccGAA Acgagga B | 89.67 | UCCUCGU C UCUCAGC B | 7391 | 22897 |
| 16133 | 22944 | Hammerhead | 1595 (+) | uccuguu CUGAUGAggccguuaggccGAA Augugcc B | 90.33 | GGCACAU U AACAGGA B | 7392 | 22898 |
| 16134 | 22945 | Hammerhead | 2627 (+) | ggaagga CUGAUGAggccguuaggccGAA Aggaugc B | 82.54 | GCAUCCU C UCCUUCC B | 7393 | 22899 |
| 16135 | 22946 | Hammerhead | 6598 (+) | cuccacg CUGAUGAggccguuaggccGAA Acuccuc B | 78.06 | GAGGAGU A CGUGGAG B | 7394 | 22900 |
| 16136 | 22947 | Hammerhead | 9002 (+) | ggaguga CUGAUGAggccguuaggccGAA Aaugcgc B | 81.88 | GCGCAUU U UCACUCC B | 7395 | 22901 |
| 16137 | 22948 | Hammerhead | 818 (−) | gcgagcc CUGAUGAggccguuaggccGAA Acgaguc B | 88.34 | GACUCGU A GGCUCGC B | 7396 | 22902 |
| 16138 | 22949 | Hammerhead | 1440 (−) | gcuggaa CUGAUGAggccguuaggccGAA Acacuga B | 89.16 | UCAGUGU C UUCCAGC B | 7397 | 22903 |
| 16139 | 22950 | Hammerhead | 2287 (−) | aggauga CUGAUGAggccguuaggccGAA Agagagg B | 83.43 | CCUCUCU C UCAUCCU B | 7398 | 22904 |
| 16140 | 22951 | Hammerhead | 2814 (−) | ugaggag CUGAUGAggccguuaggccGAA Acgugga B | 83.25 | UCCACGU A CUCCUCA B | 7399 | 22905 |
| 16141 | 22952 | Hammerhead | 3131 (−) | gacugga CUGAUGAggccguuaggccGAA Augcacg B | 86.96 | CGUGCAU A UCCAGUC B | 7400 | 22906 |
| 16142 | 22971 | DNAzyme | 855 (+) | aggaaga GGCTAGCTACAACGA agagaaa B | 92.11 | UUUCUCU A UCUUCCU B | 7401 | 22925 |
| 16143 | 22972 | DNAzyme | 1188 (+) | ugagaga GGCTAGCTACAACGA gaggaag B | 86.38 | CUUCCUC G UCUCUCA B | 7402 | 22926 |
| 16144 | 22973 | DNAzyme | 1199 (+) | aggugaa GGCTAGCTACAACGA agcugag B | 83.15 | CUCAGCU G UUCACCU B | 7403 | 22927 |
| 16145 | 22974 | DNAzyme | 5718 (+) | cugguga GGCTAGCTACAACGA ggaggcu B | 57.82 | AGCCUCC A UCACCAG B | 7404 | 22928 |
| 16146 | 22975 | DNAzyme | 6521 (+) | uggugua GGCTAGCTACAACGA gcguuga B | 75.77 | UCAACGC A UACACCA B | 7405 | 22929 |
| 16147 | 22976 | DNAzyme | 829 (−) | aagagga GGCTAGCTACAACGA gcggcga B | 66.06 | UCGCCGC G UCCUCUU B | 7406 | 22930 |
| 16148 | 22977 | DNAzyme | 1613 (−) | aaggaga GGCTAGCTACAACGA gaaggcg B | 71.28 | CGCCUUC A UCUCCUU B | 7407 | 22931 |
| 16149 | 22978 | DNAzyme | 2290 (−) | aggagga GGCTAGCTACAACGA gagagag B | 61.60 | CUCUCUC A UCCUCCU B | 7408 | 22932 |
| 16150 | 22979 | DNAzyme | 2812 (−) | aggagua GGCTAGCTACAACGA guggagg B | 85.53 | CCUCCAC G UACUCCU B | 7409 | 22933 |
| 16151 | 22980 | DNAzyme | 7123 (−) | guuguga GGCTAGCTACAACGA uuggagg B | 34.60 | CCUCCAA A UCACAAC B | 7410 | 22934 |
| 16102 | 22719 | G-cleaver | 1438 (+) | uggaaga uGAUg gcaugcacuaugcgCg acugaga B | 69.88 | UCUCAGU G UCUUCCA B | 7411 | 22813 |
| 16103 | 22720 | G-cleaver | 4591 (+) | ggagagg uGAUg gcaugcacuaugcgCg auauaca B | 77.74 | UGUAUAU G CCUCUCC B | 7412 | 22814 |
| 16104 | 22721 | G-cleaver | 5270 (+) | ucuaagg uGAUg gcaugcacuaugcgCg acacggu B | 47.37 | ACCGUGU G CCUUAGA B | 7413 | 22815 |
| 16105 | 22722 | G-cleaver | 6223 (+) | accaccu uGAUg gcaugcacuaugcgCg acuccac B | 75.84 | GUGGAGU G AGGUGGU B | 7414 | 22816 |
| 16106 | 22723 | G-cleaver | 7741 (+) | acagguu uGAUg gcaugcacuaugcgCg aacucgu B | 61.58 | ACGAGUU G AACCUGU B | 7415 | 22817 |
| 16107 | 22724 | G-cleaver | 884 (−) | ggauggu uGAUg gcaugcacuaugcgCg agacagg B | 65.16 | CCUGUCU G ACCAUCC B | 7416 | 22818 |
| 16108 | 22725 | G-cleaver | 2492 (−) | ggaaaag uGAUg gcaugcacuaugcgCg aacagga B | 94.66 | UCCUGUU G CUUUUCC B | 7417 | 22819 |
| 16109 | 22726 | G-cleaver | 2639 (−) | agaagaa uGAUg gcaugcacuaugcgCg acgagga B | 82.14 | UCCUCGU G UUCUUCU B | 7418 | 22820 |
| 16110 | 22727 | G-cleaver | 4082 (−) | ggacgag uGAUg gcauGcacuaugcgCg acuuugu B | 67.20 | ACAAAGU G CUCGUCG B | 7419 | 22821 |
| 16111 | 22728 | G-cleaver | 8958 (−) | gguaggu uGAUg gcauGcacuaugcgCg aaguggc B | 81.06 | GCCACUU G ACCUACC B | 7420 | 22822 |
| 1611 | 222747 | Zinzyme | 1188 (+) | ugagaga gccgaaaggCgagugaGGuCu gaggaag B | 66.11 | CUUCCUC G UCUCUCA B | 7402 | 22841 |
| 16113 | 22748 | Zinzyme | 1199 (+) | aggugaa gccgaaaggCgagugaGGuCu agcugag B | 80.28 | CUCAGCU G UUCACCU B | 7403 | 22842 |
| 16114 | 22749 | Zinzyme | 2492 (+) | ggaaaag gccgaaaggCgagugaGGuCu aacagga B | 90.80 | UCCUGUU G CUUUUCC B | 7417 | 22843 |
| 16115 | 22750 | Zinzyme | 2639 (+) | agaagaa gccgaaaggCgagugaGGuCu acgagga B | 80.64 | UCCUCGU G UUCUUCU B | 7418 | 22844 |
| 16116 | 22751 | Zinzyme | 8799 (+) | gaguuga gccgaaaggCgagugagGuCu uggagug B | 14.85 | CACUCCA G UCAACUC B | 7421 | 22845 |
| 16117 | 22752 | Zinzyme | 829 (−) | aagagga gccgaaaggCgagugaGGuCu gcggcga B | 27.83 | UCGCCGC G UCCUCUU B | 7406 | 22846 |
| 16118 | 22753 | Zinzyme | 1438 (−) | uggaaga gccgaaaggCgagugaGGuCu acugaga B | 89.39 | UCUCAGU G UCUUCCA B | 7411 | 22847 |
| 16119 | 22754 | Zinzyme | 2812 (−) | aggagua gccgaaaggCgagugaGGuCu guggagg 8 | 50.40 | CCUCCAC G UACUCCU B | 7409 | 22848 |
| 16120 | 22755 | Zinzyme | 3790 (−) | cgaagca gccgaaaggCgagugaGGuCu augugga B | 81.10 | UCCACAU G UGCUUCG B | 7422 | 22849 |
| 16121 | 22756 | Zinzyme | 8602 (−) | uggaaga gccgaaaggCgagugaGGuCu ggcguga B | 73.47 | UCACGCC G UCUUCCA B | 7423 | 22850 |
| 16122 | 22775 | Inozyme | 858 (+) | aagagga CUGAUGAggccguuaggccGAA Iauagag B | 87.74 | CUCUAUC U UCCUCUU B | 7424 | 22869 |
| 16123 | 22776 | Inozyme | 1198 (+) | ggugaac CUGAUGAggccguuaggccGAA Icugaga B | 84.55 | UCUCAGC U GUUCACC B | 7425 | 22870 |
| 16124 | 22777 | Inozyme | 2630 (+) | cgaggaa CUGAUGAggccguuaggccGAA lagagga B | 90.12 | UCCUCUC C UUCCUCG B | 7426 | 22871 |
| 16125 | 22778 | Inozyme | 5714 (+) | ugaugga CUGAUGAggccguuaggccGAA Icuguga B | 83.77 | UCACAGG C UCCAUCA B | 7427 | 22872 |
| 16126 | 22779 | Inozyme | 8130 (+) | ugaggaa CUGAUGAggccguuaggccGAA Iguggag B | 82.22 | CUCCACC C UUCCUCA B | 7428 | 22873 |
| 16127 | 22780 | Inozyme | 1433 (−) | gacacug CUGAUGAggccguuaggccGAA lacacca B | 87.33 | UGGUGUC U CAGUGUC B | 7429 | 22874 |
| 16128 | 22781 | Inozyme | 1610 (−) | gagauga CUGAUGAggccguuaggccGAA Igcgaag B | 70.67 | CUUCGCC U UCAUCUC B | 7430 | 22875 |
| 16129 | 22782 | Inozyme | 2286 (−) | ggaugag CUGAUGAggccguuaggccGAA Iagaggu B | 78.83 | ACCUCUC U CUCAUCC B | 7431 | 22876 |
| 16130 | 22783 | Inozyme | 3339 (−) | ugugcag CUGAUGAggccguuaggccGAA Igaugaa B | 86.93 | UUCAUCC A CUGCACA B | 7432 | 22877 |
| 16131 | 22784 | Inozyme | 6869 (−) | uggauga CUGAUGAggccguuaggccGAA Icuguug B | 90.41 | CAACAGC A UCAUCCA B | 7433 | 22878 |

In vitro cleavage in 50 mM Tris-Cl, pH 8.0, 40 mM Mg₂₊at 37°, using trace substrate, and enzymatic nucleic acid concentration of 500 nM or greater.
UPPER CASE = RIBO
UNDERLINED = DEOXY
lower case = 2′-O-methyl
B = inverted deoxyabasic
C = 2′-amino C
(+/−) = plus strand/minus strand of HCV genome

What we claim is:

1. A short interfering RNA (siRNA) molecule that down-regulates expression of hepatitis B virus (HBV) RNA, wherein said siRNA comprises nucleotide sequence complementary to said HBV RNA or a portion thereof.

2. The siNA molecule of claim 1, wherein said siRNA molecule is double stranded.

3. The siRNA molecule of claim 2, wherein each strand of said siRNA comprises about 21 nucleotides.

4. The siRNA molecule of claim 2, wherein each strand of said siRNA comprises a 3′-nucleotide overhang.

5. The siRNA molecule of claim 4, wherein said 3′-nucleotide overhang comprises two nucleotides.

6. The siRNA molecule of claim 1, wherein said siRNA molecule is single stranded.

7. The siRNA molecule of claim 1, wherein said siRNA molecule comprises one or more chemically modified nucleotides.

8. The siRNA molecule of claim 7, wherein said chemically modified nucleotide is a 2′-O-alkyl nucleotide.

9. The siRNA molecule of claim 8, wherein said 2′-O-alkyl nucleotides is a 2′-O-methyl nucleotide.

10. The siRNA molecule of claim 8, wherein said 2′-O-alkyl nucleotides is a 2′-O-allyl nucleotide.

11. The siRNA molecule of claim 7, wherein said chemically modified nucleotide is a 2′-deoxy-2′-fluoro nucleotide.

12. The siRNA molecule of claim 7, wherein said chemically modified nucleotide is a 2′-deoxy nucleotide.

13. The siRNA molecule of claim 7, wherein said chemically modified nucleotide comprises one or more phosphorothioate internucleotide linkages.

14. The siRNA molecule of claim 7, wherein said chemically modified nucleotide is a 2′-O-alkyl nucleotide, 2′-deoxy-2′-fluoro nucleotide, 2′-deoxy nucleotide, phosphorothioate containing nucleotide, or any combination thereof.

15. The siRNA molecule of claim 7, wherein said siRNA comprises one or more 2′-O-alkyl and one or more 2′-deoxy-2′-fluoro nucleotides.

16. The siRNA molecule of claim 7, wherein said siRNA comprises one or more 2′-deoxy and one or more 2′-deoxy-2′-fluoro nucleotides.

17. The siRNA molecule of claim 1, wherein said siRNA comprises a terminal cap modification.

18. The siRNA molecule of claim 17, wherein said terminal cap modification comprises an inverted abasic moiety.

19. The siRNA molecule of claim 17, wherein said terminal cap modification is at the 3′-end of said siRNA.

20. The siRNA molecule of claim 1, wherein said siRNA is conjugated to a biologically active molecule.

21. The siRNA molecule of claim 20, wherein said siRNA molecule is conjugated to said biologically active molecule via a biodegradable linker.

22. The siRNA molecule of claim 20, wherein said biologically active molecule comprises a vitamin.

23. The siRNA molecule of claim 20, wherein said biologically active molecule comprises an antibody.

24. The siRNA molecule of claim 20, wherein said biologically active molecule comprises a hormone.
